(19) United States
(12) Patent Application Publication
(10) Pub. No.: US 2025/0313864 A1
(43) Pub. Date: Oct. 9, 2025

(54) TYPE V CAS PROTEINS AND APPLICATIONS THEREOF

(71) Applicant: Alia Therapeutics Srl, Milano (IT)

(72) Inventors: Antonio CASINI, Trento (IT); Antonio CARUSILLO, Trento (IT); Veronica PINAMONTI, Trento (IT); Matteo CICIANI, Schio (IT); Maddalena BOSETTI, Terre d'adige (IT)

(73) Assignee: Alia Therapeutics Srl, Milano (IT)

(21) Appl. No.: 19/232,045

(22) Filed: Jun. 9, 2025

Related U.S. Application Data
(63) Continuation of application No. PCT/EP2025/059128, filed on Apr. 3, 2025.
(60) Provisional application No. 63/574,354, filed on Apr. 4, 2024.

(51) Int. Cl. C12N 15/90; C12N 9/22; C12N 15/11

(52) U.S. Cl. CPC C12N 15/907; C12N 9/226; C12N 15/11; C07K 2319/09; C12N 2310/20

(57) ABSTRACT

Type V Cas proteins, for example Type V Cas proteins referred to as ZWGD, ZJHK, ZIKV, ZZFT, YYAN, ZZGY, ZKBG, ZZKD, ZXPB, ZPPX, ZXHQ, ZQKH, ZRGM, ZTAE, ZSQQ, ZSYN, ZRBH, ZWPU, ZZQE, and ZRXE Type V Cas proteins; gRNAs for Type V Cas proteins; systems comprising Type V Cas proteins and gRNAs; nucleic acids encoding the Type V Cas proteins, gRNAs and systems; particles comprising the foregoing; pharmaceutical compositions of the foregoing; and uses of the foregoing, for example to alter the genomic DNA of a cell.

### 1. CROSS-REFERENCE TO RELATED APPLICATIONS

This application is a continuation of PCT application no. PCT/EP2025/059128, filed Apr. 3, 2025, which claims the priority benefit of U.S. provisional application No. 63/574,354, filed Apr. 4, 2024, the contents of each of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Mar. 25, 2025, is named ALA-013WO_SL.xml and is 679,601 bytes in size.

### 3. BACKGROUND

CRISPR-Cas systems (Clustered Regularly Interspaced Short Palindromic Repeats-CRISPR associated proteins) are powerful tools with the potential to treat a variety of genetic diseases. The CRISPR-Cas systems are classified into two classes (Class 1 and 2) that are subdivided into six types (Type 1 through VI). Class 1 (Type I, III and IV) systems use multiple Cas proteins in their CRISPR ribonucleoprotein effector nucleases, and Class 2 systems (Type II, V and VI) use a single Cas protein. Cas9, belonging to Class 2 Type II CRISPR-Cas system, is the most extensively used tool for genome editing.

However, there are some challenges in using CRISPR-Cas9 systems. For example, packaging a large Cas protein such as SpCas9 together with a guide RNA into a single AAV vector (Adeno-associated viral vectors) can be challenging due to the limited packaging capacity of AAVs. Type V Cas proteins such as Cas12a target T-rich sequences, which in principle allow Type V Cas proteins to access different genomic regions as compared to Cas9. Type V Cas proteins typically produce staggered ends when it creates a double stranded DNA cut (while Cas9 creates a blund end), which may be an advantage in certain situations such as during gene insersions and substitutions. Type V Cas proteins also typically produce mid sized deletions at the target site (generally tens of nucleotides) allowing for the removal of target sequences locally (e.g. binding sites for transcription factors, splice sites, etc). In comparason, Cas9 produces relatively small indels (generally insertion or deletion of a few nucleotides). Type V Cas proteins such as Cas12a are typically capable of processing their own crRNA from larger transcripts, which can make multiplexing easier.

Thus, there is a need for new Cas nucleases, especially Type V Cas nucleases.

### 4. SUMMARY

This disclosure is based, in part, on the discovery of a Type V Cas protein from an unclassified bacterium from the Candidatus Saccharibacteria phylum (referred to herein as “wildtype ZWGD type V Cas”); a Type V Cas protein from an unclassified bacterium from the Clostridiaceae family (referred to herein as “wildtype ZJHK type V Cas”); a Type V Cas protein from an unclassified bacterium from the Firmucutes phylum (referred to herein as “wildtype ZIKV type V Cas”); a Type V Cas protein from an unclassified bacterium from the Bacteroidota phylum (referred to herein as “wildtype ZZFT type V Cas”); a Type V Cas protein from an unclassified bacterium from the Firmicutes phylum (referred to herein as “wildtype YYAN type V Cas”); a Type V Cas protein from an unclassified bacterium from the Succinivibrionaceae family (referred to herein as “wildtype ZZGY type V Cas”); a Type V Cas protein from an unclassified bacterium from the Muribaculaceae family (referred to herein as “wildtype ZKBG type V Cas”); a Type V Cas protein from Mogibacterium kristiansenii (referred to herein as “wildtype ZZKD type V Cas”); a Type V Cas protein from an unclassified bacterium from the Bacteroidales order (referred to herein as “wildtype ZXPB type V Cas”); a Type V Cas protein from an unclassified bacterium from the Prevotellaceae family (referred to herein as “wildtype ZPPX type V Cas”); a Type V Cas protein from an unclassified bacterium from the phylum Candidatus Roizmanbacteria (referred to herein as “wildtype ZXHQ type V Cas”); a Type V Cas protein from an unclassified bacterium from the phylum Bacteroidota (referred to herein as “wildtype ZQKH type V Cas”); a Type V Cas protein from an unclassified bacterium from the phylum Firmicutes (referred to herein as “wildtype ZRGM type V Cas”); a Type V Cas protein from an unclassified bacterium from the phylum Kiritimatiellaeota (referred to herein as “wildtype ZTAE type V Cas”); a Type V Cas protein from an unclassified bacterium from the phylum Fibrobacteres (referred to herein as “wildtype ZSQQ type V Cas”); a Type V Cas protein from an unclassified bacterium from the phylum Firmicutes (referred to herein as “wildtype ZSYN type V Cas”); a Type V Cas protein from an unclassified bacterium from the phylum Firmicutes (referred to herein as “wildtype ZRBH type V Cas”); a Type V Cas protein from an unclassified bacterium from the phylum Bacteroidota (referred to herein as “wildtype ZWPU type V Cas”); a Type V Cas protein from an unclassified bacterium from the Prevotellaceae family (referred to herein as “wildtype ZZQE type V Cas”); and a Type V Cas protein from an unclassified bacterium from the phylum Bacteroidota (referred to herein as “wildtype ZRXE type V Cas”).

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:1 (such proteins referred to herein as “ZWGD Type V Cas proteins”). Exemplary ZWGD Type V Cas protein sequences are set forth in SEQ ID NO:1, SEQ ID NO:2, and SEQ ID NO:3.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:7 (such proteins referred to herein as “ZJHK Type V Cas proteins”). Exemplary ZJHK Type V Cas protein sequences are set forth in SEQ ID NO:7, SEQ ID NO:8, and SEQ ID NO:9.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO: 13 (such proteins referred to herein as “ZIKV Type V Cas proteins”). Exemplary ZIKV Type V Cas protein sequences are set forth in SEQ ID NO: 13, SEQ ID NO: 14, and SEQ ID NO: 15.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:19 (such proteins referred to herein as “ZZFT Type V Cas proteins”). Exemplary ZZFT Type V Cas protein sequences are set forth in SEQ ID NO: 19, SEQ ID NO: 20, and SEQ ID NO:21.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:25 (such proteins referred to herein as “YYAN Type V Cas proteins”). Exemplary YYAN Type V Cas protein sequences are set forth in SEQ ID NO:25, SEQ ID NO: 26, and SEQ ID NO:27.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:31 (such proteins referred to herein as “ZZGY Type V Cas proteins”). Exemplary ZZGY Type V Cas protein sequences are set forth in SEQ ID NO:31, SEQ ID NO: 32, and SEQ ID NO:33.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:37 (such proteins referred to herein as “ZKBG Type V Cas proteins”). Exemplary ZKBG Type V Cas protein sequences are set forth in SEQ ID NO:37, SEQ ID NO: 38, and SEQ ID NO:39.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:43 (such proteins referred to herein as “ZZKD Type V Cas proteins”). Exemplary ZZKD Type V Cas protein sequences are set forth in SEQ ID NO:43, SEQ ID NO: 44, and SEQ ID NO:45.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:49 (such proteins referred to herein as “ZXPB Type V Cas proteins”). Exemplary ZXPB Type V Cas protein sequences are set forth in SEQ ID NO:49, SEQ ID NO: 50, and SEQ ID NO:51.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:55 (such proteins referred to herein as “ZPPX Type V Cas proteins”). Exemplary ZPPX Type V Cas protein sequences are set forth in SEQ ID NO:55, SEQ ID NO: 56, and SEQ ID NO:57.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:61 (such proteins referred to herein as “ZXHQ Type V Cas proteins”). Exemplary ZXHQ Type V Cas protein sequences are set forth in SEQ ID NO:61, SEQ ID NO: 62, and SEQ ID NO:63.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:67 (such proteins referred to herein as “ZQKH Type V Cas proteins”). Exemplary ZQKH Type V Cas protein sequences are set forth in SEQ ID NO:67, SEQ ID NO: 68, and SEQ ID NO:69.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:73 (such proteins referred to herein as “ZRGM Type V Cas proteins”). Exemplary ZRGM Type V Cas protein sequences are set forth in SEQ ID NO:73, SEQ ID NO: 74, and SEQ ID NO:75.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:79 (such proteins referred to herein as “ZTAE Type V Cas proteins”). Exemplary ZTAE Type V Cas protein sequences are set forth in SEQ ID NO:79, SEQ ID NO: 80, and SEQ ID NO: 81.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:85 (such proteins referred to herein as “ZSQQ Type V Cas proteins”). Exemplary ZSQQ Type V Cas protein sequences are set forth in SEQ ID NO:85, SEQ ID NO: 86, and SEQ ID NO:87.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:91 (such proteins referred to herein as “ZSYN Type V Cas proteins”). Exemplary ZSYN Type V Cas protein sequences are set forth in SEQ ID NO:91, SEQ ID NO: 92, and SEQ ID NO:93.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:97 (such proteins referred to herein as “ZRBH Type V Cas proteins”). Exemplary ZRBH Type V Cas protein sequences are set forth in SEQ ID NO:97, SEQ ID NO: 98, and SEQ ID NO:99.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:103 (such proteins referred to herein as “ZWPU Type V Cas proteins”). Exemplary ZWPU Type V Cas protein sequences are set forth in SEQ ID NO: 103, SEQ ID NO: 104, and SEQ ID NO: 105.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO: 109 (such proteins referred to herein as “ZZQE Type V Cas proteins”). Exemplary ZZQE Type V Cas protein sequences are set forth in SEQ ID NO: 109, SEQ ID NO: 110, and SEQ ID NO: 111.

In one aspect, the disclosure provides Type V Cas proteins whose amino acid sequence comprises an amino acid sequence that is at least 50% identical (e.g., at least 55%, at least 60%, at least 65%, at least 70%, at least 75%, at least 85%, at least 90%, at least 95%, at least 98%, at least 99% identical, or 100% identical) to SEQ ID NO:115 (such proteins referred to herein as “ZRXE Type V Cas proteins”). Exemplary ZRXE Type V Cas protein sequences are set forth in SEQ ID NO: 115, SEQ ID NO: 116, and SEQ ID NO: 117.

In another aspect, the disclosure provides Type V Cas proteins comprising an amino acid sequence having at least 50% (e.g., at least 55%, at least 60%, at least 65%, at least 70%, at least 75%, at least 85%, at least 90%, at least 95%, at least 98%, at least 99%, or 100%) sequence identity to a WED-1 domain, REC1 domain, REC2 domain, WED-II domain, PI domain, WED-III domain, RuvC-I domain, BH domain, RuvC-II domain, NUC domain, or RuvC-III domain of a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, or a ZRXE Type V Cas protein.

In some embodiments, a Type V Cas protein of the disclosure is a chimeric Type V Cas protein, for example, comprising one or more domains from a ZWGD, ZJHK, ZIKV, ZZFT, YYAN, ZZGY, ZKBG, ZZKD, ZXPB, ZPPX, ZXHQ, ZQKH, ZRGM, ZTAE, ZSQQ, ZSYN, ZRBH, ZWPU, ZZQE, and/or ZRXE Type V Cas protein(s) and one or more domains from a different Type V Cas protein such as AsCas12a.

In some embodiments, the Type V Cas proteins of the disclosure are in the form of a fusion protein, for example, comprising a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, or a ZRXE Type V Cas protein sequence fused to one or more additional amino acid sequences, for example, one or more nuclear localization signals and/or one or more tags. Other exemplary fusion partners can enable base editing (e.g., where the fusion partner is nucleoside deaminase) or prime editing (e.g., where the fusion partner is a reverse transcriptase).

Exemplary features of Type V Cas proteins of the disclosure are described in Section 6.2 and specific embodiments 1 to 329 and 660 to 671, infra.

In further aspects, the disclosure provides guide (gRNA) molecules and combinations of two or more gRNA molecules. In various embodiments, the disclosure provides gRNAs that can be used with a ZWGD, ZJHK, ZIKV, ZZFT, YYAN, ZZGY, ZKBG, ZZKD, ZXPB, ZPPX, ZXHQ, ZQKH, ZRGM, ZTAE, ZSQQ, ZSYN, ZRBH, ZWPU, ZZQE, or ZRXE Type V Cas protein of the disclosure. Exemplary features of the gRNAs and combinations of gRNAs of the disclosure of the disclosure are described in Section 6.3 and specific embodiments 330 to 578, infra.

In further aspects, the disclosure provides systems comprising a Type V Cas protein of the disclosure and one or more gRNAs. For example, a system can comprise a ribonucleoprotein (RNP) comprising a Type V Cas protein complexed with a gRNA. Exemplary features of systems are described in Section 6.4 and specific embodiments 579 to 594, infra.

In another aspect, the disclosure provides nucleic acids and pluralities of nucleic acids encoding a Type V Cas protein of the disclosure and, optionally, a gRNA. In some embodiments, the nucleic acids comprise a Type V Cas protein of the disclosure operably linked to a heterologous promoter, e.g., a mammalian promoter, for example a human promoter.

In another aspect, the disclosure provides nucleic acids encoding a gRNA, and, optionally, a Type V Cas protein, for example a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, or a ZRXE Type V Cas protein. Exemplary features of nucleic acids and pluralities of nucleic acids are described in Section 6.5 and specific embodiments 595 to 659, infra.

In further aspects, the disclosure provides particles comprising the Type V Cas proteins, gRNAs, nucleic acids, and systems of the disclosure. Exemplary features of particles of the disclosure are described in Section 6.6 and specific embodiments 672 to 687, infra.

In another aspect, the disclosure provides cells and populations of cells containing or contacted with a Type V Cas protein, gRNA, nucleic acid, plurality of nucleic acids, system, or particle of the disclosure. Exemplary features of such cells and cell populations are described in Section 6.6 and specific embodiments 689 to 699 and 737, infra.

In another aspect, the disclosure provides pharmaceutical compositions comprising a Type V Cas protein, gRNA, nucleic acid, plurality of nucleic acids, system, particle, cell, or population of cells together with one or more excipients. Exemplary features of pharmaceutical compositions are described in Section 6.7 and specific embodiment 688, infra.

In another aspect, the disclosure provides methods of altering cells (e.g., editing the genome of a cell) using the Type V Cas proteins, gRNAs, nucleic acids, systems, particles, and pharmaceutical compositions of the disclosure. Cells altered according to the methods of the disclosure can be used, for example, to treat subjects having a disease or disorder, e.g., genetic disease or disorder. Features of exemplary methods of altering cells are described in Section 6.8 and specific embodiments 700 to 736, infra.

In another aspect, the disclosure provides methods of detecting a target nucleic acid using the Type V Cas proteins, gRNAs, and systems of the disclosure, and use of the foregoing in such methods. Features of exemplary methods of detecting target nucleic acids, and Type V Cas proteins, gRNAs, and systems for use in methods of detecting a target nucleic acid are described in Section 6.9 and specific embodiments 738 to 740, infra.

### 5. BRIEF DESCRIPTION OF THE FIGURES

FIGS. 1A-1E illustrate exemplary Type V-A Cas protein crRNAs (corresponding DNA sequences shown). Schematic representation of the hairpin structure generated for visualization using RNAplot after in silico folding using RNAalifold v2.4.17 of the crRNA scaffolds (not including the spacer sequence) for ZWGD Type V-A Cas protein (FIG. 1A), ZJHK Type V-A Cas protein (FIG. 1B), ZIKV Type V-A Cas protein (FIG. 1C), ZZFT Type V-A Cas protein (FIG. 1D) and YYAN Type V-A Cas protein (FIG. 1E) are shown. Figures disclose SEQ ID NOS 390-394, respectively, in order of appearance.

FIGS. 2A-2E illustrate exemplary Type V-A Cas protein crRNAs (corresponding DNA sequences shown). Schematic representation of the hairpin structure generated for visualization using RNAplot after in silico folding using RNAalifold v2.4.17 of the crRNA scaffolds (not including the spacer sequence) for ZZGY Type V-A Cas protein (FIG. 2A), ZKBG Type V-A Cas protein (FIG. 2B), ZZKD Type V-A Cas protein (FIG. 2C), ZXPB Type V-A Cas protein (FIG. 2D) or ZPPX Type V-A Cas protein (FIG. 2E). Figures disclose SEQ ID NOS 395-399, respectively, in order of appearance.

FIGS. 3A-3E illustrate in silico predicted PAM specificities for ZWGD, ZJHK, ZIKV, ZZFT and YYAN Type V-A Cas proteins. PAM sequence logos for ZWGD (FIG. 3A), ZJHK (FIG. 3B), ZIKV (FIG. 3C), ZZFT (FIG. 3D) and YYAN (FIG. 3E) Type V-A Cas proteins are shown.

FIGS. 4A-4E illustrate in silico predicted PAM specificities for ZZGY, ZKBG, ZZKD, ZXPB and ZPPX Type V-A Cas proteins. PAM sequence logos for ZZGY (FIG. 4A), ZKGB (FIG. 4B), ZZKD (FIG. 4C), ZXPB (FIG. 4D) and ZPPX (FIG. 4E) Type V-A Cas proteins are shown.

FIG. 5 illustrates activity of Type V-A Cas proteins against an EGFP reporter in mammalian cells. The activity of the selected Type V-A Cas proteins was evaluated after transient electroporation of plasmids encoding each nuclease together with the indicated guide RNAs in U2OS cells stably expressing EGFP. For each Cas protein, 2 different gRNAs targeting the same two positions of the EGFP coding sequence were evaluated. Loss of EGFP fluorescence, expressed as % of EGFP-negative cells, was measured by cytofluorimetry. Data presented as mean±SEM of n≥2 biologically independent runs. Untreated U2OS cells (U2OS sample) are included as a measurement of the background loss of fluorescence.

FIGS. 6A-6C illustrate activity of ZZKD Type V-A Cas protein against benchmark endogenous genomic loci in mammalian cells. The activity of ZZKD Type V-A Cas protein was evaluated after transient electroporation of plasmids encoding each nuclease together with the indicated guide RNAs in U2OS cells. Several gRNAs targeting the TRAC (FIG. 6A), B2M (FIG. 6B) and PD1 (FIG. 6C) benchmark loci were evaluated. Editing activity was measured by Sanger chromatogram deconvolution 3 days after transfection. Data presented as mean±SEM of n≥2 biologically independent runs.

FIGS. 7A-7E illustrate exemplary Type V-A Cas protein crRNAs (corresponding DNA sequences shown). Schematic representation of the hairpin structure generated for visualization using RNAplot after in silico folding using RNAalifold v2.4.17 of the crRNA scaffolds (not including the spacer sequence) for ZXHQ Type V-A Cas protein (FIG. 7A), ZQKH Type V-A Cas protein (FIG. 7B), ZRGM Type V-A Cas protein (FIG. 7C), ZTAE Type V-A Cas protein (FIG. 7D) and ZSQQ Type V-A Cas protein (FIG. 7E) are shown. Figures disclose SEQ ID NOS 400-404, respectively, in order of appearance.

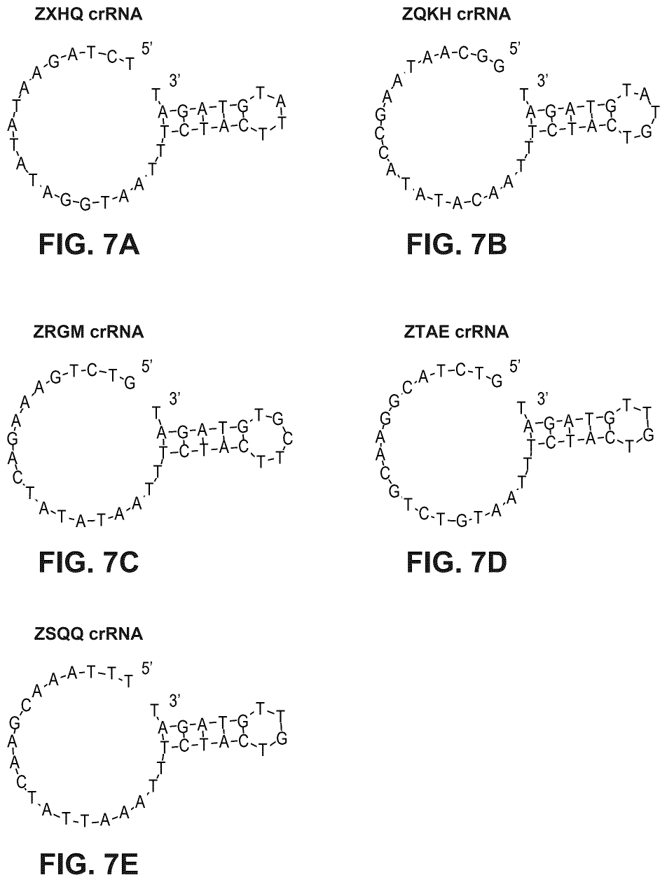

FIGS. 8A-8E illustrate exemplary Type V-A Cas protein crRNAs (corresponding DNA sequences shown). Schematic representation of the hairpin structure generated for visualization using RNAplot after in silico folding using RNAalifold v2.4.17 of the crRNA scaffolds (not including the spacer sequence) for ZSYN Type V-A Cas protein (FIG. 8A), ZRBH Type V-A Cas protein (FIG. 8B), ZWPU Type V-A Cas protein (FIG. 8C), ZZQE Type V-A Cas protein (FIG. 8D) and ZRXE Type V-A Cas protein (FIG. 8E) are shown. Figures disclose SEQ ID NOS 405-409, respectively, in order of appearance.

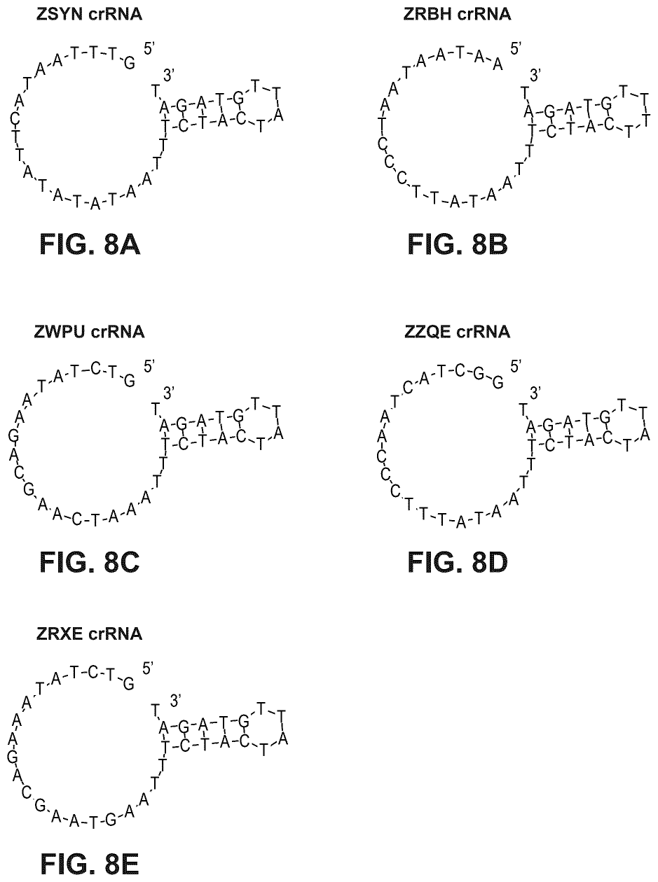

FIG. 9 illustrates in silico prediction of ZZQE Type V-A Cas protein PAM specificity. PAM sequence logo for ZZQE Type V-A Cas protein is shown.

FIG. 10 shows activity of novel Type V-A Cas proteins in human cells. Evaluation of the activity of novel Type V-A Cas protein after transient electroporation in U2OS-EGFP cells. Two different guide RNAs were evaluated (target sequences are common for all proteins) and EGFP downregulation was measured by flow cytometry 5 days post-electroporation. A non-transfected control sample has been included to measure the assay background (NT Ctrl). 23nt spacers were used. Data represented as mean±SD of n=2 independent biological replicates.

FIG. 11 shows activity of selected Type V-A Cas proteins towards endogenous genomic loci in human cells. The editing activity of ZZKD, ZRGM and ZZQE Type V-A Cas proteins was evaluated for the benchmark TRAC-g3, B2M-g2 and PD1-g2 genomic loci after transient transfection in HEK293T cells. Given the PAM compatibility among the different proteins the same spacers were used (23nt in length). For ZZKD activity on the TRAC locus, data represented as mean±SD of n=3 independent biological replicates.

FIGS. 12A-12C show in vitro analysis of PAM preferences of ZZKD Type V-A Cas protein. A PAM sequence logo is shown in FIG. 12A and PAM heatmap is shown in FIG. 12B for ZZKD Type V-A Cas protein FIG. 12C shows validation of the PAM preferences by measurement of indel formation after transient transfection of HEK293T cells using crRNAs associated with PAMs shown to be preferentially cut by the PAM assay. The PAM associated with each guide is reported on the graph. Data represented as mean±SD of n≥2 independent biological replicates.

FIGS. 13A-13D show analysis of PAM preferences of ZRGM and ZZQE Type V-A Cas proteins. A PAM sequence logo is shown in FIG. 13A and a PAM heatmap is shown in FIG. 13B for ZRGM Type V-A Cas protein. A PAM sequence logo is shown in FIG. 13C and a PAM heatmap is shown in FIG. 13D for ZZQE Type V-A Cas protein.

FIGS. 14A-14B illustrate in vitro determination of the double strand break profile of ZZKD Type V-A Cas protein. In vitro cleavage reactions using a PCR-generated target (TRAC-g3) and recombinant ZZKD Type V-A Cas protein were run on an agarose gel and the separated fragments were independently Sanger sequenced using a forward and a reverse primer to sequence both DNA strands. Based on the drop in the chromatographic signal in the two sequencing reactions (FIG. 14A) it was possible to determine that ZZKD type V-A Cas protein produces a 6 nucleotide staggered cut, as indicated by the solid lines in the scheme shown in FIG. 14B. Figure discloses SEQ ID NOS 410-411, 410, and 412, respectively, in order of appearance.

FIG. 15 shows an evaluation of alternative nuclear localization signal (NLS) designs to improve the activity of ZZKD Type V-A Cas protein. FIG. 15 plots indel formation at the TRAC locus (g3) after transient transfection of HEK293T cells with alternative versions of ZZKD Type V-A Cas proteins characterized by different nuclear localization signal sequences positioned either at the N- or the C-terminus of the protein, as indicated on the graph. The amino acid sequence of each evaluated NLS is reported in the figure. Data represented as mean±SD of n≥2 independent biological replicates. Figure discloses SEQ ID NOS 179, 122, 180, and 125, respectively, in order of appearance.

FIGS. 16A-16C show alternative crRNA scaffolds for selected Type V-A Cas proteins. Schematic representation of the hairpin structure generated for visualization using the RNAfold webserver (www.unafold.org) of the crRNA trimmed scaffolds (not including the spacer sequence) for ZZKD Type V-A Cas protein (FIG. 16A) (SEQ ID NO:211), ZZQE Type V-A Cas protein (FIG. 16B) (SEQ ID NO:212) and ZRGM Type V-A Cas protein (FIG. 16C) (SEQ ID NO:213).

FIGS. 17A-17B show the activity of alternative crRNA scaffolds for selected Type V-A Cas proteins. FIG. 17A shows indel formation measured after transient transfection of HEK293T cells with alternative versions (full-length or trimmed) of the crRNAs targeting the TRAC-g3 locus for ZZKD, ZZQE and ZRGM Type V-A Cas proteins. FIG. 17B shows indel formation measured after transient transfection of HEK293T cells with alternative versions (full-length or trimmed) of ZZKD Type V-A Cas protein crRNAs targeting the BCL11A, TRAC, AAVS1 and B2M loci, as indicated on the graph. Data represented as mean±SD of n=2 independent biological replicates.

FIGS. 18A-18B illustrate the effect of alternative spacer lengths on ZZKD Type V-A Cas protein editing activity. Indel formation in HEK293T cells after transient transfection of ZZKD Type V-A Cas protein in combination with families of crRNAs characterized by different spacer lengths (from 20nt to 24nt) targeting either the Match6 (FIG. 18A) or the TRAC locus (g3, FIG. 18B). Data represented as mean±SD of n=2 independent biological replicates.

FIG. 19 shows a side-by-side comparison of ZZKD Type V-A Cas protein activity with AsCs12a Ultra. The figure shows a violin plot summarizing the editing activity of ZZKD Type V-A Cas protein and AsCas12a Ultra on a panel of endogenous genomic loci (TRAC, PD1, B2M, EMX1, AAVS1, BCL11a, PCSK9, Match6, VEGFA) after transient transfection of HEK292T cells, using crRNAs for the two nucleases that overlap on each locus. Each point on the graph represents the mean of n=2 independent runs except for B2M-g1_21nt for AsCas12a Ultra (n=1).

FIGS. 20A-20D show activity of ZZKD Type V-A Cas protein in subsaturating conditions. Titration curves obtained by measuring indel formation at the BCL11A-g4 (FIG. 20A), VEGFA-g1 (FIG. 20B), B2M-g1_21nt (FIG. 20C) and B2M-g2_21nt (FIG. 20D) target sites after a 2-fold serial dilution of the amount of ZZKD and crRNA plasmids transiently transfected in HEK293T cells. The activity of AsCas12a Ultra was measured in the same study conditions as a benchmark and is reported on each graph. Data represented as mean±SD of n=2 independent biological replicates.

FIGS. 21A-21C show activity of ZZKD Type V-A Cas after direct ribonucleoprotein delivery in human cell lines. Indel formation after ZZKD Type V-A Cas RNP electroporation in U2OS cells to target either the TRAC-g3 locus (FIG. 21A) or the B2M-g2 locus (FIG. 21B). Cells were also transfected with plasmids expressing ZZKD and its crRNA as a positive control. IVT, in vitro transcribed crRNA; syn, unmodified chemically synthesized crRNA; AltR, chemically synthesized crRNA including commercially available AltR modifications from IDT. FIG. 21C shows the results of a titration study in U2OS cells delivering different amounts of recombinant ZZKD and cognate crRNA targeting the B2M-g2 locus by electroporation. The amount (pmol) of recombinant protein and crRNA used in each condition is indicated below each bar. Data represented as mean±SD of n≥2 independent biological replicates, except for B2M-g2 IVT and panel (FIG. 21C) where only one replicate is available.

FIG. 22 shows activity of ZZKD Type V-A Cas after direct ribonucleoprotein delivery in primary human T cells. The figure shows percentage of TRAC-negative cells measured by flow cytometry after ZZKD Type V-A Cas RNP electroporation in commercial human primary T cells to target the TRAC-g3 locus.

### 6. DETAILED DESCRIPTION

In one aspect, the disclosure provides Type V Cas proteins, e.g., a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, and a ZRXE Type V Cas protein. Type V Cas proteins of the disclosure can be in the form of fusion proteins. Unless required otherwise by context, disclosures relating to Type V Cas proteins encompass Type V Cas proteins which are not fusion proteins and Type V Cas proteins which are in the form of fusion proteins (e.g., Type V Cas protein comprising one or more nuclear localization signals and/or one or more tags).

In some embodiments, a Type V Cas protein of the disclosure comprises an amino acid sequence having at least 50% (e.g., at least 55%, at least 60%, at least 65%, at least 70%, at least 75%, at least 85%, at least 90%, at least 95%, at least 98%, at least 99%, or 100%) sequence identity to a WED-1 domain, REC1 domain, REC2 domain, WED-II domain, PI domain, WED-III domain, RuvC-I domain, BH domain, RuvC-II domain, NUC domain, or RuvC-III domain of a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, or a ZRXE Type V Cas protein.

In some embodiments, a Type V Cas protein of the disclosure is a chimeric Type V Cas protein, for example, comprising one or more domains from a ZWGD Type V Cas protein and/or a ZJHK Type V Cas protein and/or a ZIKV Type V Cas protein and/or a ZZFT Type V Cas protein and/or a YYAN Type V Cas protein and/or a ZZGY Type V Cas protein and/or a ZKBG Type V Cas protein and/or a ZZKD Type V Cas protein and/or a ZXPB Type V Cas protein and/or a ZPPX Type V Cas protein and/or a ZXHQ Type V Cas protein and/or a ZQKH Type V Cas protein and/or a ZRGM Type V Cas protein and/or a ZTAE Type V Cas protein and/or a ZSQQ Type V Cas protein and/or a ZSYN Type V Cas protein and/or a ZRBH Type V Cas protein and/or a ZWPU Type V Cas protein and/or a ZZQE Type V Cas protein and/or a ZRXE Type V Cas protein, and one or more domains from a different Type V Cas protein such as AsCas12a.

Exemplary features of Type V Cas proteins of the disclosure are described in Section 6.2.

In further aspects, the disclosure provides guide (gRNA) molecules and combinations of guide RNA molecules, for example combinations of two or more gRNAs. Exemplary features of the gRNAs and combinations of gRNAs of the disclosure are further described in Section 6.3.

In further aspects, the disclosure provides systems comprising a Type V Cas protein of the disclosure and one or more gRNAs. Exemplary features of systems are described in Section 6.4.

In further aspects, the disclosure provides nucleic acids and pluralities of nucleic acids encoding a Type V Cas protein of the disclosure and, optionally, a gRNA, and provides nucleic acids encoding a gRNA, of the disclosure and, optionally, a Type V Cas protein. Exemplary features of nucleic and pluralities of nucleic acids of the disclosure are described in Section 6.5.

In further aspects, the disclosure provides particles comprising the Type V Cas proteins, gRNAs, nucleic acids, and systems of the disclosure. Exemplary features of particles of the disclosure are described in Section 6.6.

In another aspect, the disclosure provides cells and populations of cells containing or contacted with a Type V Cas protein, gRNA, nucleic acid, plurality of nucleic acids, system, or particle of the disclosure. Exemplary features of such cells and cell populations are described in Section 6.6.

In another aspect, the disclosure provides pharmaceutical compositions comprising a Type V Cas protein, gRNA, nucleic acid, plurality of nucleic acids, system, particle, cell, or population of cells together with one or more excipients. Exemplary features of pharmaceutical compositions are described in Section 6.7.

In another aspect, the disclosure provides methods of altering cells (e.g., editing the genome of a cell) using the Type V Cas proteins, gRNAs, nucleic acids, systems, particles, and pharmaceutical compositions of the disclosure. Features of exemplary methods of altering cells are described in Section 6.8.

### 6.1. Definitions

Unless defined otherwise, all technical and scientific terms used herein have the same meaning as commonly understood to one of ordinary skill in the art to which this invention belongs. The following definitions are provided for the full understanding of terms used in this specification.

As used in the specification and claims, the singular form “a,” “an,” and “the” include plural references unless the context clearly dictates otherwise. For example, the term “an agent” includes a plurality of agents, including mixtures thereof.

Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

AsCas12a refers to a Cas12a protein having the following amino acid sequence:

(SEQ ID NO: 121)
MTQFEGFTNLYQVSKTLRFELIPQGKTLKHIQEQGFIEEDKARNDHYKELKPIIDRIYKTYADQCLQLVQLD
WENLSAAIDSYRKEKTEETRNALIEEQATYRNAIHDYFIGRTDNLTDAINKRHAEIYKGLFKAELFNGKVLK
QLGTVTTTEHENALLRSFDKFTTYFSGFYENRKNVFSAEDISTAIPHRIVQDNFPKFKENCHIFTRLITAVP
SLREHFENVKKAIGIFVSTSIEEVFSFPFYNQLLTQTQIDLYNQLLGGISREAGTEKIKGLNEVLNLAIQKND
ETAHIIASLPHRFIPLFKQILSDRNTLSFILEEFKSDEEVIQSFCKYKTLLRNENVLETAEALFNELNSIDLTHI
FISHKKLETISSALCDHWDTLRNALYERRISELTGKITKSAKEKVQRSLKHEDINLQEIISAAGKELSEAFKQ
KTSEILSHAHAALDQPLPTTLKKQEEKEILKSQLDSLLGLYHLLDWFAVDESNEVDPEFSARLTGIKLEME
PSLSFYNKARNYATKKPYSVEKFKLNFQMPTLASGWDVNKEKNNGAILFVKNGLYYLGIMPKQKGRYKA
LSFEPTEKTSEGFDKMYYDYFPDAAKMIPKCSTQLKAVTAHFQTHTTPILLSNNFIEPLEITKEIYDLNNPE
KEPKKFQTAYAKKTGDQKGYREALCKWIDFTRDFLSKYTKTTSIDLSSLRPSSQYKDLGEYYAELNPLLY
HISFQRIAEKEIMDAVETGKLYLFQIYNKDFAKGHHGKPNLHTLYWTGLFSPENLAKTSIKLNGQAELFYR
PKSRMKRMAHRLGEKMLNKKLKDQKTPIPDTLYQELYDYVNHRLSHDLSDEARALLPNVITKEVSHEIIKD
RRFTSDKFFFHVPITLNYQAANSPSKFNQRVNAYLKEHPETPIIGIDRGERNLIYITVIDSTGKILEQRSLNTI
QQFDYQKKLDNREKERVAARQAWSVVGTIKDLKQGYLSQVIHEIVDLMIHYQAVVVLENLNFGFKSKRTG
IAEKAVYQQFEKMLIDKLNCLVLKDYPAEKVGGVLNPYQLTDQFTSFAKMGTQSGFLFYVPAPYTSKIDPL
TGFVDPFVWKTIKNHESRKHFLEGFDFLHYDVKTGDFILHFKMNRNLSFQRGLPGFMPAWDIVFEKNET
QFDAKGTPFIAGKRIVPVIENHRFTGRYRDLYPANELIALLEEKGIVFRDGSNILPKLLENDDSHAIDTMVAL
IRSVLQMRNSNAATGEDYINSPVRDLNGVCFDSRFQNPEWPMDADANGAYHIALKGQLLLNHLKESKDL
KLQNGISNQDWLAYIQELRN

A Type V Cas protein refers to a wild-type or engineered Type V Cas protein. Engineered Type V Cas proteins can also be referred to as Type V Cas variants. For the avoidance of doubt, any disclosure pertaining to a “Type V Cas” or “Type V Cas protein” pertains to wild-type Type V Cas proteins and Type V Cas variants, unless the context dictates otherwise. A Type V Cas protein can have nuclease activity or be catalytically inactive (e.g., as in a dCas).

As used herein, the percentage identity between two nucleotide sequences or between two amino acid sequences is calculated by multiplying the number of matches between a pair of aligned sequences by 100, and dividing by the length of the aligned region. Identity scoring only counts perfect matches and does not consider the degree of similarity of amino acids to one another, nor does it consider substitutions or deletions as matches. For calculation of the percent sequence identity (% sequence identity), two sequences are aligned using the EMBOSS Needle Pairwise Sequence Alignment software tool based on the Needleman and Wunsch algorithm (available at www.ebi.ac.uk/jdispatcher/psa/emboss_needle) with the following parameters: Matrix: BLOSUM62 (for protein sequences) or DNAfull (for DNA sequences); Gap Open: 10; Gap Extend: 0.5; End Gap Penalty: false; End Gap Open: 10; and End Gap Extend: 0.5.

Guide RNA molecule (gRNA) refers to an RNA capable of forming a complex with a Type V Cas protein and which can direct the Type V Cas protein to a target DNA. gRNAs typically comprise a spacer of 15 to 30 nucleotides in length. gRNAs of the disclosure typically comprise a crRNA scaffold region at the 5′ end of the molecule and a spacer at the 3′ end of the molecule. Various non-limiting examples of crRNA scaffolds are described in Section 6.3.

An gRNA can in some embodiments comprise no uracil base at the 3′ end of the gRNA sequence. Alternatively, a gRNA can comprise one or more uracil bases at the 3′ end of the sgRNA sequence. For example, a gRNA can comprise 1 uracil (U) at the 3′ end of the gRNA sequence, 2 uracil (UU) at the 3′ end of the gRNA sequence, 3 uracil (UUU) at the 3′ end of the gRNA sequence, 4 uracil (UUUU) at the 3′ end of the gRNA sequence, 5 uracil (UUUUU) at the 3′ end of the gRNA sequence, 6 uracil (UUUUUU) at the 3′ end of the gRNA sequence, 7 uracil (UUUUUUU) at the 3′ end of the gRNA sequence, or 8 uracil (UUUUUUUU) at the 3′ end of the gRNA sequence. Different length stretches of uracil can be appended at the 3′ end of a gRNA as terminators.

A gRNA can in some embodiments comprise a 5′ guanine (G) at it's 5′ end. A 5′-G can promote efficient transcription from a U6 promoter.

Peptide, protein, and polypeptide are used interchangeably to refer to a natural or synthetic molecule comprising two or more amino acids linked by the carboxyl group of one amino acid to the alpha amino group of another. The amino acids may be natural or synthetic, and can contain chemical modifications such as disulfide bridges, substitution of radioisotopes, phosphorylation, substrate chelation (e.g., chelation of iron or copper atoms), glycosylation, acetylation, formylation, amidation, biotinylation, and a wide range of other modifications. A polypeptide may be attached to other molecules, for instance molecules required for function. Examples of molecules which may be attached to a polypeptide include, without limitation, cofactors, polynucleotides, lipids, metal ions, phosphate, etc. Non-limiting examples of polypeptides include peptide fragments, denatured/unstructured polypeptides, polypeptides having quaternary or aggregated structures, etc. There is expressly no requirement that a polypeptide must contain an intended function; a polypeptide can be functional, non-functional, function for unexpected/unintended purposes, or have unknown function. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Polynucleotide and oligonucleotide are used interchangeably and refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, primers and gRNAs. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. A polynucleotide is composed of a specific sequence of four nucleotide bases: adenine (A); cytosine (C); guanine (G); thymine (T); and uracil (U) for thymine (T) when the polynucleotide is RNA. Thus, the term “nucleotide sequence” is the alphabetical representation of a polynucleotide molecule. The letters used in polynucleotide sequences described herein correspond to IUPAC notation. For example, the letter “N” in a nucleotide sequence represents a nucleotide which can be A, T, C, or G in a DNA sequence, or A, U, C, or G in a RNA sequence; the letter “R” in a nucleotide sequence represents a nucleotide which can be A or G; the letter “V” in a nucleotide sequence represents a nucleotide which can be A, C, or G; and the letter “Y” in a nucleotide sequence represents a nucleotide which can be C or T.

Protospacer adjacent motif (PAM) refers to a DNA sequence upstream (e.g., immediately upstream) of a target sequence on the non-target strand recognized by a Type V Cas protein. A PAM sequence is located 5′ of the target sequence on the non-target strand.

Spacer refers to a region of a gRNA molecule which is partially or fully complementary to a target sequence found in the + or − strand of genomic DNA. When complexed with a Type V Cas protein, the gRNA directs the Type V Cas to the target sequence in the genomic DNA. A spacer of a Type V Cas gRNA is typically 15 to 30 nucleotides in length (e.g., 20-25 nucleotides). The nucleotide sequence of a spacer can be, but is not necessarily, fully complementary to the target sequence. For example, a spacer can contain one or more mismatches with a target sequence, e.g., the spacer can comprise one, two, or three mismatches with the target sequence.

### 6.2. Type V Cas Proteins

### 6.2.1. ZWGD Type V Cas Proteins

In one aspect, the disclosure provides ZWGD Type V Cas proteins. ZWGD Type V Cas proteins can be further classified as Type V-A Cas proteins. The ZWGD Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:1. In some embodiments, the ZWGD Type V Cas proteins comprise an amino acid sequence that is at least 95%, at least 96%, at least 97%, at least 98%, or at least 99% identical to SEQ ID NO: 1. In some embodiments, a ZWGD Type V Cas protein comprises an amino acid sequence that is identical to SEQ ID NO: 1.

Exemplary ZWGD Type V Cas protein sequences and nucleotide sequences encoding exemplary ZWGD Type V Cas proteins are set forth in Table 1A.

TABLE 1A
ZWGD Type V Cas Sequences

| | | SEQ ID |
|---|---|---|
| Name | Sequence | NO. |
| Wildtype | VSEKENTPTFNSLTNLYSVSKTLRFELRPQYSTLDHIKDDQIVDKGEELKNHYKTFKKILD | 1 |
| amino acid | QVFSRIINDSLDKTYLDQKYISTYQDLVFKHRDRLTDKDRAELKALKETLKKQIDKSLDHK | |
| sequence | DKKAIFSDPVNFLIDNESDFADLIGDNRPSIEAFNRQKGYLSGYLQNRANIFDHTTNETSV | |
| (without N- | AFRIVEENLAIFLNNRLTLQHFFEKVADKDGLLKFLQETLSQLGFKLKLEDLLSLDYFNRT | |
| terminal | LSQPGIDQYNLLISGKALEDGKKMQGINEVLNQYLQQHQEEKLHKIKLKQLYKQILSESK | |
| methionine) | TESFTLDFVEDNKGLAAMLLQFIDFVNKLIEEKMLLLDMIQGLKDSSVSSEFLSRLYLERK | |
| | NIKRLSNFIYKDYGYIEQSLEENFLSTIEGKITKKALEEHRKQDAFTIHEILVALQKQQYEK | |
| | DGALESADHLLLPGVVDFLYQNLDCKHSTLLEKVGSEKQPLLDLFNEKQLLEGQDAESH | |
| | ASKYSDRPFNDHEIKVVKTALDFYKNLQSNFAIFQIPDENLKLDSEFYSEFDEFYQGLKNI | |
| | IPVYNKSRNFLTKKPFSTEKTKLIFNNPQLLDGWSKSKESDCLGTIFIKDGKYYVGIINSAT | |
| | NAKNTLFEPNNFANFDQKQYFEKMNLFFLSDLKRDFPKKYFSEKWHNQHPVPADLREK | |
| | YDYYRIDEHKDERKNDLKYHHQLIAYYQDCLKKDTEWQIYQFKYKAPEEYSDVNEFLSE | |
| | LTPNTYKMEFNKIPAEYIKKLVDDGKLYFFQIYSKDFSEFAKGKPNLHTLYLKAVFDQKNA | |
| | EEFNYNYKISGSAEIFYRPASIETRVTHPKNQPIKNKNKNNPKAESVFQYDLCKDRRYMS | |
| | DKFFLHLPIELNRIPLLANDSSVNSMVNQVVSSRNQNYFLGIDRGERHLIYLVLIDQNGRII | |
| | KQQTLNQITSSYQEKANNQTVEVITDYHDLLNDKEKLRKKNLQEWQSVENIKELKAGYL | |
| | SNWVNEIGKIIVEYQPVIMLENLNTGFKNSRIKIEKQVYQKFEKALIDKFNYFMRKDLDSSA | |
| | IGGLYHALQLTKEYSKQYNGKQNGIIYYIPASYTSNIDPTTGFISAFIQTRYENVEKTKSLIE | |
| | KFNDITYDAEESLFCFSADYKKFSPEAKLWQQTIWQIYTNGDRIYTFKNKEEWQSKNYIL | |
| | VEEFKDLFAKYHIDYCRDLKAQILSQTDASFFKQFLFLLRLTLQMRNSRTTELNGTDADT | |
| | KKRENDYIISPVKNQYGKFYDSRKDYVDWPENADANGAYNIARKGLIMLKHLKEGLPEK | |
| | RICDISTEEWVQFVEELNK | |
| Wildtype | MVSEKENTPTFNSLTNLYSVSKTLRFELRPQYSTLDHIKDDQIVDKGEELKNHYKTFKKIL | 2 |
| amino acid | DQVFSRIINDSLDKTYLDQKYISTYQDLVFKHRDRLTDKDRAELKALKETLKKQIDKSLDH | |
| sequence (with | KDKKAIFSDPVNFLIDNESDFADLIGDNRPSIEAFNRQKGYLSGYLQNRANIFDHTTNETS | |
| N-terminal | VAFRIVEENLAIFLNNRLTLQHFFEKVADKDGLLKFLQETLSQLGFKLKLEDLLSLDYFNR | |
| methionine) | TLSQPGIDQYNLLISGKALEDGKKMQGINEVLNQYLQQHQEEKLHKIKLKQLYKQILSES | |
| | KTESFTLDFVEDNKGLAAMLLQFIDFVNKLIEEKMLLLDMIQGLKDSSVSSEFLSRLYLER | |
| | KNIKRLSNFIYKDYGYIEQSLEENFLSTIEGKITKKALEEHRKQDAFTIHEILVALQKQQYE | |
| | KDGALESADHLLLPGVVDFLYQNLDCKHSTLLEKVGSEKQPLLDLFNEKQLLEGQDAES | |
| | HASKYSDRPFNDHEIKVVKTALDFYKNLQSNFAIFQIPDENLKLDSEFYSEFDEFYQGLK | |
| | NIIPVYNKSRNFLTKKPFSTEKTKLIFNNPQLLDGWSKSKESDCLGTIFIKDGKYYVGIINS | |
| | ATNAKNTLFEPNNFANFDQKQYFEKMNLFFLSDLKRDFPKKYFSEKWHNQHPVPADLR | |
| | EKYDYYRIDEHKDERKNDLKYHHQLIAYYQDCLKKDTEWQIYQFKYKAPEEYSDVNEFL | |
| | SELTPNTYKMEFNKIPAEYIKKLVDDGKLYFFQIYSKDFSEFAKGKPNLHTLYLKAVFDQK | |
| | NAEEFNYNYKISGSAEIFYRPASIETRVTHPKNQPIKNKNKNNPKAESVFQYDLCKDRRY | |
| | MSDKFFLHLPIELNRIPLLANDSSVNSMVNQVVSSRNQNYFLGIDRGERHLIYLVLIDQNG | |
| | RIIKQQTLNQITSSYQEKANNQTVEVITDYHDLLNDKEKLRKKNLQEWQSVENIKELKAG | |
| | YLSNVVNEIGKIIVEYQPVIMLENLNTGFKNSRIKIEKQVYQKFEKALIDKFNYFMRKDLDS | |
| | SAIGGLYHALQLTKEYSKQYNGKQNGIIYYIPASYTSNIDPTTGFISAFIQTRYENVEKTKS | |
| | LIEKFNDITYDAEESLFCFSADYKKFSPEAKLWQQTIWQIYTNGDRIYTFKNKEEWQSKN | |
| | YILVEEFKDLFAKYHIDYCRDLKAQILSQTDASFFKQFLFLLRLTLQMRNSRTTELNGTDA | |
| | DTKKRENDYIISPVKNQYGKFYDSRKDYVDWPENADANGAYNIARKGLIMLKHLKEGLP | |
| | EKRICDISTEEWVQFVEELNK | |
| Expression | MGVSEKENTPTFNSLTNLYSVSKTLRFELRPQYSTLDHIKDDQIVDKGEELKNHYKTFK | 3 |
| construct (with | KILDQVFSRIINDSLDKTYLDQKYISTYQDLVFKHRDRLTDKDRAELKALKETLKKQIDKS | |
| N-terminal | LDHKDKKAIFSDPVNFLIDNESDFADLIGDNRPSIEAFNRQKGYLSGYLQNRANIFDHTT | |
| methionine, | NETSVAFRIVEENLAIFLNNRLTLQHFFEKVADKDGLLKFLQETLSQLGFKLKLEDLLSLD | |
| V5-tag and C- | YFNRTLSQPGIDQYNLLISGKALEDGKKMQGINEVLNQYLQQHQEEKLHKIKLKQLYKQI | |
| terminal NLS) | LSESKTESFTLDFVEDNKGLAAMLLQFIDFVNKLIEEKMLLLDMIQGLKDSSVSSEFLSRL | |
| aa sequence | YLERKNIKRLSNFIYKDYGYIEQSLEENFLSTIEGKITKKALEEHRKQDAFTIHEILVALQK | |
| | QQYEKDGALESADHLLLPGVVDFLYQNLDCKHSTLLEKVGSEKQPLLDLFNEKQLLEG | |
| | QDAESHASKYSDRPFNDHEIKVVKTALDFYKNLQSNFAIFQIPDENLKLDSEFYSEFDEF | |
| | YQGLKNIIPVYNKSRNFLTKKPFSTEKTKLIFNNPQLLDGWSKSKESDCLGTIFIKDGKYY | |
| | VGIINSATNAKNTLFEPNNFANFDQKQYFEKMNLFFLSDLKRDFPKKYFSEKWHNQHPV | |
| | PADLREKYDYYRIDEHKDERKNDLKYHHQLIAYYQDCLKKDTEWQIYQFKYKAPEEYSD | |
| | VNEFLSELTPNTYKMEFNKIPAEYIKKLVDDGKLYFFQIYSKDFSEFAKGKPNLHTLYLKA | |
| | VFDQKNAEEFNYNYKISGSAEIFYRPASIETRVTHPKNQPIKNKNKNNPKAESVFQYDLC | |
| | KDRRYMSDKFFLHLPIELNRIPLLANDSSVNSMVNQVVSSRNQNYFLGIDRGERHLIYLV | |
| | LIDQNGRIIKQQTLNQITSSYQEKANNQTVEVITDYHDLLNDKEKLRKKNLQEWQSVENI | |
| | KELKAGYLSNVVNEIGKIIVEYQPVIMLENLNTGFKNSRIKIEKQVYQKFEKALIDKFNYFM | |
| | RKDLDSSAIGGLYHALQLTKEYSKQYNGKQNGIIYYIPASYTSNIDPTTGFISAFIQTRYE | |
| | NVEKTKSLIEKFNDITYDAEESLFCFSADYKKFSPEAKLWQQTIWQIYTNGDRIYTFKNK | |
| | EEWQSKNYILVEEFKDLFAKYHIDYCRDLKAQILSQTDASFFKQFLFLLRLTLQMRNSRT | |
| | TELNGTDADTKKRENDYIISPVKNQYGKFYDSRKDYVDWPENADANGAYNIARKGLIML | |
| | KHLKEGLPEKRICDISTEEWVQFVEELNKSRKRTADGSEFESPKKKRKVGSGKPIPNPL | |
| | LGLDST | |
| Wildtype | ATGGTGTCCGAAAAAGAAAATACACCAACTTTTAATAGTCTAACCAATCTCTATAGTG | 4 |
| coding | TTTCAAAGACTCTTAGATTTGAACTTAGGCCACAATATTCAACTCTAGATCACATTAA | |
| sequence (with | AGATGACCAAATTGTTGACAAAGGTGAAGAACTAAAAAACCACTACAAAACTTTCAA | |
| N-terminal | GAAAATTCTTGATCAGGTCTTTTCAAGGATCATCAACGATAGCCTAGATAAAACCTA | |
| methionine | TCTTGATCAAAAATATATTTCCACCTACCAAGATCTTGTATTCAAGCATCGAGACCGA | |
| and stop | CTAACAGACAAAGACCGTGCAGAACTAAAGGCCTTAAAAGAAACACTCAAAAAGCA | |
| codon) | GATCGACAAAAGCCTCGATCATAAAGATAAAAAAGCTATCTTCAGTGATCCCGTAAA | |
| | TTTTCTCATCGACAATGAATCGGATTTTGCTGACTTAATTGGTGATAATCGTCCTAGT | |
| | ATTGAAGCTTTCAACCGTCAAAAAGGTTATCTTTCCGGATATCTCCAAAATCGCGCA | |
| | AATATCTTCGATCACACCACAAATGAAACTTCAGTCGCGTTTCGTATTGTCGAGGAA | |
| | AACCTCGCTATCTTTTTAAATAATCGCCTCACATTACAGCATTTTTTCGAGAAAGTTG | |
| | CAGATAAAGATGGGCTATTAAAATTTTTACAAGAGACACTTTCTCAGTTAGGTTTTAA | |
| | GTTGAAACTCGAAGACCTTCTTTCCCTTGATTATTTTAATCGTACCCTATCTCAACCC | |
| | GGCATCGATCAGTATAACCTCCTAATCTCTGGCAAGGCGCTAGAAGATGGAAAGAA | |
| | AATGCAGGGAATTAATGAGGTCCTCAATCAATATCTCCAACAACATCAAGAAGAGAA | |
| | GCTACATAAAATCAAACTCAAGCAACTCTATAAGCAGATCCTCTCAGAGTCAAAAAC | |
| | TGAATCATTTACCCTTGATTTTGTGGAAGATAATAAAGGGCTTGCTGCCATGCTCCT | |
| | ACAGTTTATCGATTTTGTAAACAAGCTGATTGAAGAGAAAATGCTTCTCCTTGATATG | |
| | ATTCAGGGGCTAAAAGATAGCTCAGTTTCATCAGAATTTCTTTCACGACTCTATCTT | |
| | GAACGCAAAAACATCAAGCGTCTTTCGAATTTTATCTATAAAGATTATGGCTATATTG | |
| | AGCAATCCTTGGAAGAGAACTTTCTCTCGACAATTGAAGGCAAGATTACCAAGAAG | |
| | GCACTCGAGGAACATCGCAAACAGGATGCTTTCACAATCCATGAAATCTTAGTTGC | |
| | CCTACAAAAGCAACAATATGAAAAGGATGGAGCTCTAGAGTCCGCAGATCATCTTTT | |
| | ACTTCCTGGTGTTGTTGACTTCCTCTACCAGAATTTGGATTGCAAACACTCCACTCT | |
| | ACTTGAAAAAGTCGGGTCAGAAAAACAGCCACTACTCGACCTCTTCAACGAAAAAC | |
| | AATTATTGGAAGGTCAAGACGCAGAATCTCATGCTTCCAAATATTCTGATCGTCCAT | |
| | TCAACGACCACGAAATAAAGGTTGTTAAAACTGCTTTGGATTTTTATAAAAATCTACA | |
| | GAGTAATTTTGCGATCTTTCAAATCCCGGATGAAAACCTTAAACTAGATTCCGAATTT | |
| | TATTCCGAGTTTGATGAATTTTATCAAGGTCTCAAGAATATTATTCCAGTCTATAACA | |
| | AGTCCAGAAATTTCCTCACTAAAAAACCATTCTCAACCGAAAAGACCAAGCTCATTT | |
| | TTAACAACCCGCAACTACTTGACGGATGGAGTAAATCAAAAGAGTCAGATTGTTTAG | |
| | GCACGATTTTTATTAAAGACGGCAAATATTATGTTGGCATTATTAATAGTGCTACGAA | |
| | TGCTAAAAATACTTTATTTGAGCCTAACAATTTTGCAAACTTCGACCAAAAACAATAT | |
| | TTTGAAAAGATGAACCTTTTCTTCCTTTCGGACTTGAAGCGAGATTTTCCTAAGAAAT | |
| | ATTTTTCTGAAAAGTGGCATAATCAACACCCAGTTCCAGCCGATCTTCGTGAAAAGT | |
| | ATGATTATTATCGAATCGACGAACATAAGGATGAGCGCAAAAATGATCTAAAATATC | |
| | ATCATCAACTTATCGCCTATTATCAAGACTGTCTTAAAAAAGACACGGAATGGCAGA | |
| | TTTATCAATTCAAATATAAGGCCCCTGAAGAATATTCAGATGTCAATGAATTCTTATC | |
| | CGAGCTTACTCCAAATACCTACAAAATGGAGTTCAATAAAATCCCAGCTGAATATAT | |
| | CAAAAAGCTTGTTGATGATGGAAAATTATATTTCTTCCAAATTTATTCCAAAGATTTTT | |
| | CTGAGTTTGCAAAAGGTAAACCAAATCTCCATACTCTCTATCTAAAAGCGGTCTTTG | |
| | ATCAGAAAAATGCGGAAGAGTTCAACTATAATTATAAAATTTCTGGTAGTGCCGAAA | |
| | TCTTCTATCGTCCAGCCAGCATTGAAACTCGTGTCACTCATCCAAAAAATCAACCAA | |
| | TCAAGAATAAGAATAAAAATAATCCAAAGGCTGAATCTGTCTTCCAGTATGATCTTTG | |
| | TAAAGATCGTCGCTATATGTCAGATAAATTCTTTTTGCATCTTCCGATCGAATTAAAT | |
| | CGTATTCCGTTACTCGCTAACGACTCCTCGGTAAATAGTATGGTCAATCAAGTCGTT | |
| | AGTTCTCGTAATCAGAATTATTTCCTTGGTATTGACCGTGGCGAGAGGCATCTAATT | |
| | TATCTAGTCCTGATCGATCAAAACGGTAGAATCATTAAACAGCAAACCTTAAATCAG | |
| | ATCACTAGTTCATACCAAGAAAAAGCCAATAACCAAACGGTTGAAGTTATTACGGAT | |
| | TATCATGATCTCTTGAATGACAAAGAAAAACTGCGAAAGAAGAATCTCCAAGAGTGG | |
| | CAATCCGTCGAAAATATCAAGGAGTTAAAGGCTGGGTACCTAAGTAATGTGGTGAA | |
| | TGAAATCGGTAAGATTATCGTTGAATATCAGCCAGTTATTATGCTGGAAAATCTTAAT | |
| | ACTGGATTTAAAAACTCACGAATTAAAATTGAGAAACAGGTGTACCAGAAATTTGAG | |
| | AAGGCGCTCATTGATAAGTTTAACTACTTTATGAGAAAAGATCTCGACTCTTCAGCT | |
| | ATTGGTGGTCTCTATCACGCTTTGCAGTTGACTAAGGAATACTCTAAGCAGTACAAC | |
| | GGCAAGCAGAATGGTATCATCTACTATATTCCTGCAAGCTACACTAGTAATATTGAT | |
| | CCAACTACTGGTTTCATCTCGGCCTTTATACAGACTAGATACGAAAACGTCGAGAAA | |
| | ACAAAATCCTTAATCGAAAAGTTTAATGATATCACTTATGATGCAGAAGAATCTCTCT | |
| | TCTGCTTCTCCGCAGATTACAAGAAATTTAGTCCAGAGGCCAAGCTTTGGCAGCAG | |
| | ACGATTTGGCAGATTTATACTAATGGCGATCGTATTTATACATTTAAGAACAAAGAAG | |
| | AGTGGCAGAGCAAAAACTACATCCTCGTTGAGGAGTTCAAAGATCTCTTTGCTAAAT | |
| | ATCACATCGATTATTGCAGGGACCTTAAGGCGCAGATTCTGTCACAAACTGACGCG | |
| | AGCTTCTTCAAGCAGTTCCTCTTCTTGTTGCGACTAACCTTGCAGATGCGAAATAGT | |
| | CGCACTACCGAATTAAATGGAACTGATGCTGATACTAAAAAACGTGAGAATGATTAT | |
| | ATTATTTCTCCAGTTAAGAATCAGTATGGCAAGTTCTATGATTCCCGCAAGGATTAT | |
| | GTGGACTGGCCAGAAAATGCAGATGCAAATGGCGCATACAATATTGCCAGAAAAGG | |
| | TCTCATCATGCTAAAACACCTAAAAGAAGGTCTTCCCGAAAAACGTATCTGTGATAT | |
| | ATCGACTGAAGAATGGGTACAGTTTGTCGAAGAACTAAATAAATAG | |
| Codon | GTGTCTGAAAAGGAAAACACCCCTACCTTCAACTCTCTGACCAACCTGTACAGCGTT | 5 |
| optimized | TCTAAAACCCTGCGGTTCGAGCTGCGGCCTCAGTACAGCACCCTGGACCACATCAA | |
| coding | GGACGATCAGATCGTGGACAAGGGAGAGGAGCTAAAGAACCACTACAAGACATTC | |
| sequence (no | AAAAAAATCCTGGACCAGGTGTTCTCTCGGATCATCAACGACTCTCTGGATAAAACT | |
| N-terminal | TACCTGGATCAGAAGTACATCTCCACCTACCAGGATCTGGTGTTCAAGCACAGAGA | |
| methionine, no | TAGACTGACAGATAAGGACAGAGCCGAACTGAAGGCCCTGAAGGAGACACTGAAG | |
| stop codon) | AAGCAGATCGACAAAAGCCTGGATCACAAAGACAAGAAGGCTATCTTCTCCGACCC | |
| | TGTGAACTTCCTGATCGACAATGAGAGCGACTTCGCCGACCTGATTGGAGACAACC | |
| | GGCCCAGCATCGAGGCCTTTAACCGCCAGAAGGGATATCTGTCCGGCTACCTGCA | |
| | GAATAGAGCCAACATCTTCGATCATACAACCAACGAAACCAGCGTTGCTTTCAGAAT | |
| | CGTGGAAGAGAACCTCGCCATCTTCCTCAACAACCGCCTGACCCTGCAGCATTTCT | |
| | TCGAGAAAGTGGCCGACAAAGACGGACTGCTGAAGTTCCTGCAGGAGACACTGAG | |
| | CCAGCTGGGCTTCAAGCTGAAGCTGGAGGATCTGCTGAGCCTGGATTACTTTAACC | |
| | GGACACTGAGCCAGCCTGGCATCGACCAATACAACCTGCTGATCAGCGGAAAGGC | |
| | CCTGGAAGATGGCAAGAAGATGCAGGGCATCAATGAAGTGCTGAACCAGTACCTG | |
| | CAGCAGCACCAGGAGGAAAAGCTGCACAAAATCAAGCTGAAGCAGCTGTATAAGCA | |
| | AATCCTGAGCGAAAGCAAGACAGAGAGCTTCACGCTGGACTTCGTGGAGGACAAC | |
| | AAGGGCCTGGCCGCCATGCTGCTGCAGTTTATCGATTTCGTGAACAAGTTAATAGA | |
| | AGAGAAGATGCTGCTGCTGGATATGATCCAGGGACTGAAAGACAGCAGTGTGTCCA | |
| | GCGAGTTCTTGAGCCGGCTTTACCTGGAAAGAAAGAACATCAAGCGGCTGAGCAAC | |
| | TTCATCTACAAGGACTATGGCTATATCGAGCAGTCCCTGGAAGAAAACTTCCTGAG | |
| | CACCATCGAGGGCAAGATCACTAAGAAGGCCCTGGAAGAGCATAGAAAACAGGAC | |
| | GCCTTTACCATTCACGAGATCCTGGTCGCACTGCAGAAACAACAGTACGAAAAGGA | |
| | CGGCGCCCTAGAGAGCGCCGACCACCTGCTGCTTCCAGGCGTGGTGGATTTCCTC | |
| | TACCAAAACCTGGACTGTAAGCACAGCACGCTGCTGGAAAAGGTGGGCAGCGAGA | |
| | AGCAGCCCCTGCTGGATCTTTTCAACGAAAAGCAGCTGCTTGAGGGCCAGGACGC | |
| | CGAGTCCCACGCCTCTAAGTACAGCGATCGGCCTTTCAACGACCACGAGATCAAG | |
| | GTGGTGAAAACCGCCCTGGACTTCTACAAGAACCTGCAATCTAACTTTGCTATCTTC | |
| | CAGATCCCCGACGAAAACCTGAAGCTGGATAGCGAGTTTTACAGCGAGTTTGATGA | |
| | GTTCTACCAGGGCCTGAAAAATATTATTCCTGTGTACAACAAAAGCCGGAACTTCCT | |
| | GACAAAAAAGCCGTTCAGCACCGAAAAGACCAAACTGATCTTCAACAACCCCCAGC | |
| | TGCTCGATGGCTGGAGCAAGAGCAAGGAAAGCGACTGTCTGGGGACCATCTTCAT | |
| | CAAAGACGGCAAGTACTATGTGGGAATCATCAACAGCGCCACCAACGCTAAGAATA | |
| | CACTGTTCGAGCCTAACAACTTCGCCAATTTCGACCAAAAACAATACTTCGAGAAGA | |
| | TGAACCTGTTCTTCCTGAGCGATCTGAAGCGAGACTTCCCCAAGAAGTATTTCTCC | |
| | GAGAAGTGGCACAACCAGCACCCCGTGCCCGCTGACCTTAGAGAAAAGTACGACT | |
| | ACTACCGGATCGACGAGCATAAGGATGAGAGAAAGAATGACCTGAAATACCACCAC | |
| | CAGTTAATCGCCTACTACCAAGACTGCCTGAAAAAGGATACAGAGTGGCAGATCTA | |
| | CCAGTTCAAGTACAAGGCCCCTGAGGAGTACAGCGACGTGAACGAGTTCCTGAGT | |
| | GAACTGACCCCTAATACCTACAAGATGGAGTTCAACAAGATTCCTGCCGAGTACATT | |
| | AAGAAGCTGGTGGATGACGGCAAGCTGTACTTTTTTCAGATATACTCCAAAGACTTT | |
| | AGCGAATTTGCCAAGGGCAAGCCAAACCTGCACACCCTCTACCTGAAGGCCGTGTT | |
| | CGACCAGAAGAACGCCGAGGAGTTCAACTACAACTATAAAATATCTGGATCTGCTG | |
| | AAATCTTTTACAGACCTGCTTCTATCGAGACAAGAGTGACCCACCCTAAGAATCAGC | |
| | CTATCAAGAACAAGAACAAGAACAATCCTAAGGCTGAAAGCGTGTTCCAGTACGAC | |
| | CTGTGCAAGGACCGGCGGTACATGTCCGACAAGTTCTTCCTGCACCTTCCCATCGA | |
| | ACTTAACAGAATCCCTCTGCTGGCTAACGATTCCTCCGTGAATAGCATGGTCAACCA | |
| | GGTGGTGAGCAGCAGAAACCAGAACTACTTCCTGGGCATCGATAGAGGCGAGAGA | |
| | CACCTGATCTACCTGGTGCTGATCGACCAGAACGGTAGAATCATCAAGCAACAGAC | |
| | CCTGAATCAGATTACAAGCAGCTACCAAGAAAAGGCCAACAACCAGACAGTGGAGG | |
| | TGATCACAGACTACCACGACCTGCTGAACGACAAGGAAAAGCTCAGAAAGAAGAAT | |
| | CTTCAGGAGTGGCAGTCCGTGGAGAATATCAAAGAGCTGAAGGCCGGCTACCTGA | |
| | GCAACGTGGTCAACGAGATCGGCAAGATCATCGTGGAGTACCAGCCTGTGATCAT | |
| | GCTGGAAAACCTCAACACCGGATTTAAAAACTCAAGAATCAAGATTGAGAAGCAGG | |
| | TGTACCAGAAGTTCGAGAAGGCCTTAATCGATAAGTTCAATTACTTCATGCGGAAGG | |
| | ATCTGGACTCTAGCGCCATCGGCGGCCTGTACCACGCCCTGCAGCTGACCAAAGA | |
| | GTATAGCAAGCAGTACAACGGCAAGCAGAACGGCATCATCTACTACATCCCAGCTT | |
| | CTTACACCTCTAATATCGACCCCACCACCGGCTTTATTAGCGCCTTCATCCAGACCA | |
| | GATACGAGAACGTGGAAAAGACCAAGTCTCTGATCGAGAAATTTAATGACATCACCT | |
| | ACGACGCCGAAGAGTCGCTGTTCTGCTTCAGCGCCGATTACAAGAAATTTTCACCT | |
| | GAAGCTAAGCTGTGGCAGCAAACCATCTGGCAGATCTATACCAACGGCGACAGAAT | |
| | CTACACCTTCAAGAACAAGGAAGAGTGGCAAAGCAAGAACTACATTCTGGTGGAGG | |
| | AGTTTAAGGACCTGTTCGCCAAATACCACATCGACTATTGCAGGGACCTGAAAGCC | |
| | CAGATCCTGAGCCAGACCGACGCATCTTTTTTCAAGCAGTTTCTCTTCCTGCTGAGA | |
| | CTGACACTGCAAATGAGAAATAGTCGTACCACAGAGCTGAACGGCACCGACGCCG | |
| | ACACCAAGAAAAGAGAGAATGACTACATCATCTCTCCAGTGAAAAATCAGTACGGC | |
| | AAATTCTATGATTCCCGCAAGGACTACGTGGACTGGCCTGAGAACGCCGACGCCAA | |
| | TGGCGCCTACAACATCGCCAGAAAGGGCCTGATCATGCTGAAGCACCTGAAGGAA | |
| | GGACTGCCTGAGAAGAGGATCTGCGACATCAGCACAGAAGAATGGGTTCAGTTTGT | |
| | GGAAGAACTGAACAAG | |
| Expression | ATGggcGTGTCTGAAAAGGAAAACACCCCTACCTTCAACTCTCTGACCAACCTGTAC | 6 |
| construct (with | AGCGTTTCTAAAACCCTGCGGTTCGAGCTGCGGCCTCAGTACAGCACCCTGGACC | |
| N-terminal | ACATCAAGGACGATCAGATCGTGGACAAGGGAGAGGAGCTAAAGAACCACTACAA | |
| methionine | GACATTCAAAAAAATCCTGGACCAGGTGTTCTCTCGGATCATCAACGACTCTCTGGA | |
| and stop | TAAAACTTACCTGGATCAGAAGTACATCTCCACCTACCAGGATCTGGTGTTCAAGCA | |
| codon, | CAGAGATAGACTGACAGATAAGGACAGAGCCGAACTGAAGGCCCTGAAGGAGACA | |
| includes V5- | CTGAAGAAGCAGATCGACAAAAGCCTGGATCACAAAGACAAGAAGGCTATCTTCTC | |
| tag and C- | CGACCCTGTGAACTTCCTGATCGACAATGAGAGCGACTTCGCCGACCTGATTGGAG | |
| terminal NLS) | ACAACCGGCCCAGCATCGAGGCCTTTAACCGCCAGAAGGGATATCTGTCCGGCTA | |
| | CCTGCAGAATAGAGCCAACATCTTCGATCATACAACCAACGAAACCAGCGTTGCTTT | |
| | CAGAATCGTGGAAGAGAACCTCGCCATCTTCCTCAACAACCGCCTGACCCTGCAGC | |
| | ATTTCTTCGAGAAAGTGGCCGACAAAGACGGACTGCTGAAGTTCCTGCAGGAGACA | |
| | CTGAGCCAGCTGGGCTTCAAGCTGAAGCTGGAGGATCTGCTGAGCCTGGATTACTT | |
| | TAACCGGACACTGAGCCAGCCTGGCATCGACCAATACAACCTGCTGATCAGCGGA | |
| | AAGGCCCTGGAAGATGGCAAGAAGATGCAGGGCATCAATGAAGTGCTGAACCAGT | |
| | ACCTGCAGCAGCACCAGGAGGAAAAGCTGCACAAAATCAAGCTGAAGCAGCTGTAT | |
| | AAGCAAATCCTGAGCGAAAGCAAGACAGAGAGCTTCACGCTGGACTTCGTGGAGG | |
| | ACAACAAGGGCCTGGCCGCCATGCTGCTGCAGTTTATCGATTTCGTGAACAAGTTA | |
| | ATAGAAGAGAAGATGCTGCTGCTGGATATGATCCAGGGACTGAAAGACAGCAGTGT | |
| | GTCCAGCGAGTTCTTGAGCCGGCTTTACCTGGAAAGAAAGAACATCAAGCGGCTGA | |
| | GCAACTTCATCTACAAGGACTATGGCTATATCGAGCAGTCCCTGGAAGAAAACTTC | |
| | CTGAGCACCATCGAGGGCAAGATCACTAAGAAGGCCCTGGAAGAGCATAGAAAAC | |
| | AGGACGCCTTTACCATTCACGAGATCCTGGTCGCACTGCAGAAACAACAGTACGAA | |
| | AAGGACGGCGCCCTAGAGAGCGCCGACCACCTGCTGCTTCCAGGCGTGGTGGATT | |
| | TCCTCTACCAAAACCTGGACTGTAAGCACAGCACGCTGCTGGAAAAGGTGGGCAG | |
| | CGAGAAGCAGCCCCTGCTGGATCTTTTCAACGAAAAGCAGCTGCTTGAGGGCCAG | |
| | GACGCCGAGTCCCACGCCTCTAAGTACAGCGATCGGCCTTTCAACGACCACGAGA | |
| | TCAAGGTGGTGAAAACCGCCCTGGACTTCTACAAGAACCTGCAATCTAACTTTGCTA | |
| | TCTTCCAGATCCCCGACGAAAACCTGAAGCTGGATAGCGAGTTTTACAGCGAGTTT | |
| | GATGAGTTCTACCAGGGCCTGAAAAATATTATTCCTGTGTACAACAAAAGCCGGAAC | |
| | TTCCTGACAAAAAAGCCGTTCAGCACCGAAAAGACCAAACTGATCTTCAACAACCC | |
| | CCAGCTGCTCGATGGCTGGAGCAAGAGCAAGGAAAGCGACTGTCTGGGGACCATC | |
| | TTCATCAAAGACGGCAAGTACTATGTGGGAATCATCAACAGCGCCACCAACGCTAA | |
| | GAATACACTGTTCGAGCCTAACAACTTCGCCAATTTCGACCAAAAACAATACTTCGA | |
| | GAAGATGAACCTGTTCTTCCTGAGCGATCTGAAGCGAGACTTCCCCAAGAAGTATT | |
| | TCTCCGAGAAGTGGCACAACCAGCACCCCGTGCCCGCTGACCTTAGAGAAAAGTA | |
| | CGACTACTACCGGATCGACGAGCATAAGGATGAGAGAAAGAATGACCTGAAATACC | |
| | ACCACCAGTTAATCGCCTACTACCAAGACTGCCTGAAAAAGGATACAGAGTGGCAG | |
| | ATCTACCAGTTCAAGTACAAGGCCCCTGAGGAGTACAGCGACGTGAACGAGTTCCT | |
| | GAGTGAACTGACCCCTAATACCTACAAGATGGAGTTCAACAAGATTCCTGCCGAGT | |
| | ACATTAAGAAGCTGGTGGATGACGGCAAGCTGTACTTTTTTCAGATATACTCCAAAG | |
| | ACTTTAGCGAATTTGCCAAGGGCAAGCCAAACCTGCACACCCTCTACCTGAAGGCC | |
| | GTGTTCGACCAGAAGAACGCCGAGGAGTTCAACTACAACTATAAAATATCTGGATCT | |
| | GCTGAAATCTTTTACAGACCTGCTTCTATCGAGACAAGAGTGACCCACCCTAAGAAT | |
| | CAGCCTATCAAGAACAAGAACAAGAACAATCCTAAGGCTGAAAGCGTGTTCCAGTA | |
| | CGACCTGTGCAAGGACCGGCGGTACATGTCCGACAAGTTCTTCCTGCACCTTCCCA | |
| | TCGAACTTAACAGAATCCCTCTGCTGGCTAACGATTCCTCCGTGAATAGCATGGTCA | |
| | ACCAGGTGGTGAGCAGCAGAAACCAGAACTACTTCCTGGGCATCGATAGAGGCGA | |
| | GAGACACCTGATCTACCTGGTGCTGATCGACCAGAACGGTAGAATCATCAAGCAAC | |
| | AGACCCTGAATCAGATTACAAGCAGCTACCAAGAAAAGGCCAACAACCAGACAGTG | |
| | GAGGTGATCACAGACTACCACGACCTGCTGAACGACAAGGAAAAGCTCAGAAAGAA | |
| | GAATCTTCAGGAGTGGCAGTCCGTGGAGAATATCAAAGAGCTGAAGGCCGGCTAC | |
| | CTGAGCAACGTGGTCAACGAGATCGGCAAGATCATCGTGGAGTACCAGCCTGTGA | |
| | TCATGCTGGAAAACCTCAACACCGGATTTAAAAACTCAAGAATCAAGATTGAGAAGC | |
| | AGGTGTACCAGAAGTTCGAGAAGGCCTTAATCGATAAGTTCAATTACTTCATGCGGA | |
| | AGGATCTGGACTCTAGCGCCATCGGCGGCCTGTACCACGCCCTGCAGCTGACCAA | |
| | AGAGTATAGCAAGCAGTACAACGGCAAGCAGAACGGCATCATCTACTACATCCCAG | |
| | CTTCTTACACCTCTAATATCGACCCCACCACCGGCTTTATTAGCGCCTTCATCCAGA | |
| | CCAGATACGAGAACGTGGAAAAGACCAAGTCTCTGATCGAGAAATTTAATGACATC | |
| | ACCTACGACGCCGAAGAGTCGCTGTTCTGCTTCAGCGCCGATTACAAGAAATTTTC | |
| | ACCTGAAGCTAAGCTGTGGCAGCAAACCATCTGGCAGATCTATACCAACGGCGACA | |
| | GAATCTACACCTTCAAGAACAAGGAAGAGTGGCAAAGCAAGAACTACATTCTGGTG | |
| | GAGGAGTTTAAGGACCTGTTCGCCAAATACCACATCGACTATTGCAGGGACCTGAA | |
| | AGCCCAGATCCTGAGCCAGACCGACGCATCTTTTTTCAAGCAGTTTCTCTTCCTGCT | |
| | GAGACTGACACTGCAAATGAGAAATAGTCGTACCACAGAGCTGAACGGCACCGAC | |
| | GCCGACACCAAGAAAAGAGAGAATGACTACATCATCTCTCCAGTGAAAAATCAGTA | |
| | CGGCAAATTCTATGATTCCCGCAAGGACTACGTGGACTGGCCTGAGAACGCCGAC | |
| | GCCAATGGCGCCTACAACATCGCCAGAAAGGGCCTGATCATGCTGAAGCACCTGA | |
| | AGGAAGGACTGCCTGAGAAGAGGATCTGCGACATCAGCACAGAAGAATGGGTTCA | |
| | GTTTGTGGAAGAACTGAACAAGtctagaAAGCGGACAGCAGACGGCTCCGAATTTGAA | |
| | AGCCCTAAGAAAAAGAGAAAGGTGggatccGGCAAACCTATCCCCAATCCCCTGCTG | |
| | GGCCTGGACAGCACCTGA | |

In some embodiments a ZWGD Type V Cas protein comprises an amino acid sequence of SEQ ID NO: 1, SEQ ID NO:2, or SEQ ID NO:3. In some embodiments, a ZWGD Type V Cas protein has nickase activity, for example resulting from one or more amino acid substitutions relative to the sequence of SEQ ID NO: 1, SEQ ID NO:2, or SEQ ID NO:3. In some embodiments, the one or more amino acid substitutions providing nickase activity comprise a D891 substitution, wherein the position of the D891 substitution is defined with respect to the amino acid numbering of SEQ ID NO:2 (corresponding to amino acid 908 of SEQ ID NO: 121). In some embodiments, the one or more amino acid substitutions providing nickase activity comprise an E990 substitution, wherein the position of the E990 substitution is defined with respect to the amino acid numbering of SEQ ID NO:2 (corresponding to amino acid 993 of SEQ ID NO: 121). In some embodiments, the one or more amino acid substitutions providing nickase activity comprise a R1200 substitution, wherein the position of the R1200 substitution is defined with respect to the amino acid numbering of SEQ ID NO:2 (corresponding to amino acid 1226 of SEQ ID NO: 121). In some embodiments, the one or more amino acid substitutions providing nickase activity comprise a D1248 substitution, wherein the position of the D1248 substitution is defined with respect to the amino acid numbering of SEQ ID NO:2 (corresponding to amino acid 1263 of SEQ ID NO: 121). In some embodiments, a ZWGD Type V Cas protein is catalytically inactive, for example due to a R1200 substitution in combination with a D891 substitution, a E990 substitution, and/or D1248 substitution.

### 6.2.2. ZJHK Type V Cas Proteins

In one aspect, the disclosure provides ZJHK Type V Cas proteins. ZJHK Type V Cas proteins can be further classified as Type V-A Cas proteins. The ZJHK Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:7. In some embodiments, the ZJHK Type V Cas proteins comprise an amino acid sequence that is at least 95%, at least 96%, at least 97%, at least 98%, or at least 99% identical to SEQ ID NO:7. In some embodiments, a ZJHK Type V Cas protein comprises an amino acid sequence that is identical to SEQ ID NO:7.

Exemplary ZJHK Type V Cas protein sequences and nucleotide sequences encoding exemplary ZJHK Type V Cas proteins are set forth in Table 1B.

TABLE 1B
ZJHK Type V Cas Sequences

| | | SEQ ID |
|---|---|---|
| Name | Sequence | NO. |
| Wildtype | KSIYENFIGLESKNLTLRFALNPEAKTQENLKLYWDKLRDEERDRAYPIVKKILDKE | 7 |
| amino acid | YQQLISEGLKLLENQNVLDWTELAEYIRTSDLSKKKKEDKRLRKLIAQNLKAHPLV | |
| sequence | DKLKVKNAFGKNGYLETLPLGKEEKEAVKVFAGFGGFFNNYNKNRENYFSTEEK | |
| (without N- | STAIANRIVNENFSKHFSNVEIVTKIQKEVPELIQIVEAQFKGYDTIFTVNGYNTALS | |
| terminal | QAGIDTYNEMVAIWNKEANLYAQKAGKLPDGHPLKKKRNYLLSALFKQIGSEKEH | |
| methionine) | LIQIDRFDGDEEVIEALTGVKKMLQEADVFEKLNMLVEDMENWDYSKIYLSAQSLS | |
| | NVSVFLNNLYEDERENSWNYLDNVLREKWQIELQGKKKGTDLEEAIRKKKQSFY | |
| | SIEELQEAVNAIEETDKCYNVSKWLLGAMKSERVIEEKKKDVEDFCTQWKNERNS | |
| | LKETDITALKEYLEQWIFLARYCKSFYANGIEKKEKDEAFYHILEDVLYVLDEVIYFY | |
| | NKVRNYVTKKPYSLEKMHLKFGHNELANGWSVNKEENYGTAILRRNGKYYLAITN | |
| | SLNKKMSIPTQLESTGNNYEKMVLNVFPNVFRMIPKCTTGRNDVKSCFERKEPNE | |
| | YFFIDTPEFVNPFKVTREEYELNKITYDGVKKWQSDYSKNTQDEKGYKEAVTKWI | |
| | QFCMRFLQSYKSTAIYDYSTLQQPEKYETVDSFYHDVEKILYECHFEYVPANKIEQ | |
| | LEEEGRIFLFQIYNKDFSENRRPDSKKNLHTLYWEALFSEENRKAKVIQLNGKAEI | |
| | FRREKSIEHPIVHKAGEVLVNKRTKDGEPIPDDIYKDLSNYFNGRNVTSEKEEYKE | |
| | CLDKVYTSTKKYDITKDKRFTETKYEFHVPITLNYQADGVKYLNQKILHVLRDNPD | |
| | VNIIGLDRGERNLISYVVLNREGKIVNNQQGSFNIVGKMDYQKKLYQKEKNRDKE | |
| | RKTWKNIETIKDLKEGYISQWVHELTDMAIRNNAIIVMEDLNFGFKRGRTKVERQV | |
| | YQKFELALLKKLHYLVTDKTEGEAMLKPGGVLQGYQLAREVKTLKEIGKQCGCVF | |
| | YVPPGYTSKIDPTTGFVDVFNMSGVTNREKKKAFFEKFDNMFYDEKRDMFGFSF | |
| | NYEKFTTYQSSYRNDWTVYSNGSKYVWNSLKRTDELIDVTKELKLLFEKYAIDYR | |
| | NEALFEQIMSQDTDKNNADFWNKLFWYFRVLLRLRNSSDELDQIVSPVLNQNGE | |
| | FFETPKKITEKSYLSDYPMDADTNGAYHIALKGLYLIQEKIADESVDLDNKLPKDFY | |
| | KISNAEWFMFRQKEK | |
| Wildtype | MKSIYENFIGLESKNLTLRFALNPEAKTQENLKLYWDKLRDEERDRAYPIVKKILDK | 8 |
| amino acid | EYQQLISEGLKLLENQNVLDWTELAEYIRTSDLSKKKKEDKRLRKLIAQNLKAHPL | |
| sequence (with | VDKLKVKNAFGKNGYLETLPLGKEEKEAVKVFAGFGGFFNNYNKNRENYFSTEE | |
| N-terminal | KSTAIANRIVNENFSKHFSNVEIVTKIQKEVPELIQIVEAQFKGYDTIFTVNGYNTAL | |
| methionine) | SQAGIDTYNEMVAIWNKEANLYAQKAGKLPDGHPLKKKRNYLLSALFKQIGSEKE | |
| | HLIQIDRFDGDEEVIEALTGVKKMLQEADVFEKLNMLVEDMENWDYSKIYLSAQSL | |
| | SNVSVFLNNLYEDERENSWNYLDNVLREKWQIELQGKKKGTDLEEAIRKKKQSF | |
| | YSIEELQEAVNAIEETDKCYNVSKWLLGAMKSERVIEEKKKDVEDFCTQWKNERN | |
| | SLKETDITALKEYLEQWIFLARYCKSFYANGIEKKEKDEAFYHILEDVLYVLDEVIYF | |
| | YNKVRNYVTKKPYSLEKMHLKFGHNELANGWSVNKEENYGTAILRRNGKYYLAIT | |
| | NSLNKKMSIPTQLESTGNNYEKMVLNVFPNVFRMIPKCTTGRNDVKSCFERKEPN | |
| | EYFFIDTPEFVNPFKVTREEYELNKITYDGVKKWQSDYSKNTQDEKGYKEAVTKW | |
| | IQFCMRFLQSYKSTAIYDYSTLQQPEKYETVDSFYHDVEKILYECHFEYVPANKIE | |
| | QLEEEGRIFLFQIYNKDFSENRRPDSKKNLHTLYWEALFSEENRKAKVIQLNGKAE | |
| | IFRREKSIEHPIVHKAGEVLVNKRTKDGEPIPDDIYKDLSNYFNGRNVTSEKEEYKE | |
| | CLDKVYTSTKKYDITKDKRFTETKYEFHVPITLNYQADGVKYLNQKILHVLRDNPD | |
| | VNIIGLDRGERNLISYVVLNREGKIVNNQQGSFNIVGKMDYQKKLYQKEKNRDKE | |
| | RKTWKNIETIKDLKEGYISQWVHELTDMAIRNNAIIVMEDLNFGFKRGRTKVERQV | |
| | YQKFELALLKKLHYLVTDKTEGEAMLKPGGVLQGYQLAREVKTLKEIGKQCGCVF | |
| | YVPPGYTSKIDPTTGFVDVFNMSGVTNREKKKAFFEKFDNMFYDEKRDMFGFSF | |
| | NYEKFTTYQSSYRNDWTVYSNGSKYVWNSLKRTDELIDVTKELKLLFEKYAIDYR | |
| | NEALFEQIMSQDTDKNNADFWNKLFWYFRVLLRLRNSSDELDQIVSPVLNQNGE | |
| | FFETPKKITEKSYLSDYPMDADTNGAYHIALKGLYLIQEKIADESVDLDNKLPKDFY | |
| | KISNAEWFMFRQKEK | |
| Expression | MGKSIYENFIGLESKNLTLRFALNPEAKTQENLKLYWDKLRDEERDRAYPIVKKILD | 9 |
| construct (with | KEYQQLISEGLKLLENQNVLDWTELAEYIRTSDLSKKKKEDKRLRKLIAQNLKAHP | |
| N-terminal | LVDKLKVKNAFGKNGYLETLPLGKEEKEAVKVFAGFGGFFNNYNKNRENYFSTE | |
| methionine, | EKSTAIANRIVNENFSKHFSNVEIVTKIQKEVPELIQIVEAQFKGYDTIFTVNGYNTA | |
| V5-tag and C- | LSQAGIDTYNEMVAIWNKEANLYAQKAGKLPDGHPLKKKRNYLLSALFKQIGSEK | |
| terminal NLS) | EHLIQIDRFDGDEEVIEALTGVKKMLQEADVFEKLNMLVEDMENWDYSKIYLSAQ | |
| aa sequence | SLSNVSVFLNNLYEDERENSWNYLDNVLREKWQIELQGKKKGTDLEEAIRKKKQS | |
| | FYSIEELQEAVNAIEETDKCYNVSKWLLGAMKSERVIEEKKKDVEDFCTQWKNER | |
| | NSLKETDITALKEYLEQWIFLARYCKSFYANGIEKKEKDEAFYHILEDVLYVLDEVIY | |
| | FYNKVRNYVTKKPYSLEKMHLKFGHNELANGWSVNKEENYGTAILRRNGKYYLAI | |
| | TNSLNKKMSIPTQLESTGNNYEKMVLNVFPNVFRMIPKCTTGRNDVKSCFERKEP | |
| | NEYFFIDTPEFVNPFKVTREEYELNKITYDGVKKWQSDYSKNTQDEKGYKEAVTK | |
| | WIQFCMRFLQSYKSTAIYDYSTLQQPEKYETVDSFYHDVEKILYECHFEYVPANKI | |
| | EQLEEEGRIFLFQIYNKDFSENRRPDSKKNLHTLYWEALFSEENRKAKVIQLNGKA | |
| | EIFRREKSIEHPIVHKAGEVLVNKRTKDGEPIPDDIYKDLSNYFNGRNVTSEKEEYK | |
| | ECLDKVYTSTKKYDITKDKRFTETKYEFHVPITLNYQADGVKYLNQKILHVLRDNP | |
| | DVNIIGLDRGERNLISYVVLNREGKIVNNQQGSFNIVGKMDYQKKLYQKEKNRDK | |
| | ERKTWKNIETIKDLKEGYISQVVHELTDMAIRNNAIIVMEDLNFGFKRGRTKVERQ | |
| | VYQKFELALLKKLHYLVTDKTEGEAMLKPGGVLQGYQLAREVKTLKEIGKQCGCV | |
| | FYVPPGYTSKIDPTTGFVDVFNMSGVTNREKKKAFFEKFDNMFYDEKRDMFGFS | |
| | FNYEKFTTYQSSYRNDWTVYSNGSKYVWNSLKRTDELIDVTKELKLLFEKYAIDY | |
| | RNEALFEQIMSQDTDKNNADFWNKLFWYFRVLLRLRNSSDELDQIVSPVLNQNG | |
| | EFFETPKKITEKSYLSDYPMDADTNGAYHIALKGLYLIQEKIADESVDLDNKLPKDF | |
| | YKISNAEWFMFRQKEKSRKRTADGSEFESPKKKRKVGSGKPIPNPLLGLDST | |
| Wildtype | ATGAAAAGTATTTATGAAAATTTTATTGGATTGGAGTCAAAAAATTTGACGCTG | 10 |
| coding | CGCTTTGCGTTGAATCCAGAAGCTAAGACACAAGAAAATTTGAAGTTGTACTG | |
| sequence (with | GGACAAATTGCGTGATGAGGAGAGAGATAGGGCGTATCCAATTGTAAAAAAG | |
| N-terminal | ATATTGGATAAGGAATATCAGCAGCTGATTTCGGAAGGACTGAAATTATTAGA | |
| methionine | GAATCAGAATGTGTTGGATTGGACAGAATTAGCAGAGTATATACGGACAAGTG | |
| and stop | ATTTAAGTAAGAAGAAAAAAGAAGATAAACGCTTAAGAAAATTAATAGCACAAA | |
| codon) | ATTTAAAAGCGCATCCGTTAGTTGACAAACTGAAAGTAAAAAATGCATTTGGTA | |
| | AAAATGGCTATCTTGAAACTTTACCGTTGGGAAAAGAAGAGAAAGAGGCAGTA | |
| | AAAGTTTTTGCCGGTTTTGGCGGCTTTTTCAATAACTACAATAAAAACAGGGAA | |
| | AATTATTTTTCAACCGAGGAAAAAAGCACTGCAATCGCAAACCGAATTGTAAAT | |
| | GAAAATTTTTCAAAACATTTTTCAAATGTAGAAATAGTTACCAAAATTCAAAAGG | |
| | AAGTGCCAGAATTAATTCAAATCGTGGAAGCACAATTCAAGGGATATGATACT | |
| | ATCTTTACAGTAAATGGTTATAATACGGCATTGTCACAGGCAGGGATTGATAC | |
| | ATATAATGAGATGGTTGCAATCTGGAATAAAGAAGCAAATTTGTATGCGCAAA | |
| | AGGCAGGAAAACTTCCAGATGGACATCCGTTAAAGAAAAAGAGAAATTACTTA | |
| | TTGTCGGCATTGTTTAAACAGATTGGGAGTGAAAAGGAGCATTTGATTCAAAT | |
| | TGATAGATTTGATGGAGATGAAGAGGTGATTGAGGCATTGACGGGTGTGAAA | |
| | AAAATGCTTCAAGAGGCAGATGTATTTGAAAAATTGAATATGCTTGTGGAGGA | |
| | TATGGAGAATTGGGATTATAGTAAAATATATTTGTCAGCACAGAGTTTATCCAA | |
| | TGTTTCTGTGTTCCTAAATAATTTATATGAGGATGAACGGGAGAACTCATGGAA | |
| | TTATCTTGATAATGTCCTAAGAGAAAAATGGCAAATAGAATTACAGGGAAAGAA | |
| | AAAGGGGACAGATCTGGAAGAAGCGATTCGGAAGAAAAAACAAAGTTTCTATT | |
| | CAATAGAAGAACTTCAAGAGGCAGTGAATGCCATAGAAGAAACAGATAAATGT | |
| | TATAATGTATCTAAATGGCTTCTAGGAGCAATGAAAAGCGAAAGGGTAATAGA | |
| | AGAAAAAAAGAAGGATGTGGAAGATTTTTGCACACAGTGGAAAAATGAAAGAA | |
| | ACTCGCTGAAAGAGACAGATATAACTGCACTGAAAGAATATCTGGAGCAATGG | |
| | ATTTTTTTGGCAAGATATTGCAAATCTTTTTATGCAAATGGAATTGAAAAAAAAG | |
| | AAAAAGATGAAGCATTTTATCATATTTTAGAAGATGTGTTGTATGTTTTGGATG | |
| | AAGTAATATATTTTTATAATAAAGTTCGAAATTATGTAACGAAGAAGCCATATTC | |
| | TCTTGAAAAAATGCATTTAAAATTTGGTCATAATGAACTGGCAAATGGATGGTC | |
| | TGTTAACAAAGAAGAGAACTATGGTACGGCAATATTGAGGCGAAATGGCAAAT | |
| | ACTATTTGGCAATTACAAATTCATTGAATAAAAAGATGAGTATTCCCACTCAAT | |
| | TAGAAAGTACAGGAAATAATTATGAAAAGATGGTATTGAATGTATTCCCAAATG | |
| | TATTTCGGATGATACCAAAATGTACTACAGGAAGAAATGATGTGAAAAGTTGTT | |
| | TTGAAAGAAAAGAGCCAAATGAGTATTTCTTTATTGATACACCGGAATTTGTTA | |
| | ACCCATTTAAAGTTACGCGCGAGGAATATGAGTTAAATAAGATAACTTATGATG | |
| | GTGTTAAAAAGTGGCAATCTGATTATTCAAAAAATACGCAGGATGAAAAAGGA | |
| | TACAAAGAGGCAGTGACAAAATGGATTCAGTTTTGTATGCGCTTTTTACAATCT | |
| | TATAAGAGTACAGCAATATATGATTATTCAACTTTACAGCAACCGGAGAAATAT | |
| | GAGACGGTGGATTCTTTTTATCATGACGTTGAAAAAATATTATATGAATGTCAT | |
| | TTTGAGTACGTTCCGGCTAATAAAATAGAGCAGTTGGAAGAAGAAGGAAGAAT | |
| | TTTTCTGTTTCAGATTTACAACAAAGATTTTTCGGAAAACAGACGCCCGGACA | |
| | GCAAAAAGAATTTGCATACACTTTATTGGGAGGCATTGTTTTCAGAAGAAAATC | |
| | GGAAAGCAAAAGTGATACAATTAAATGGCAAAGCTGAAATATTTCGGAGAGAA | |
| | AAAAGCATTGAACATCCGATTGTTCATAAAGCTGGGGAAGTGTTAGTGAATAA | |
| | ACGAACGAAAGACGGGGAACCAATACCAGATGATATTTATAAAGATTTGAGCA | |
| | ACTATTTTAACGGAAGAAATGTAACATCTGAAAAGGAAGAGTATAAGGAATGT | |
| | CTGGATAAAGTGTATACTTCGACCAAAAAATATGATATTACAAAGGATAAACGT | |
| | TTTACTGAAACCAAATATGAATTTCATGTTCCGATTACCTTGAACTATCAGGCG | |
| | GACGGTGTTAAATATTTGAATCAGAAAATACTTCATGTGCTGAGGGATAATCC | |
| | AGATGTGAATATTATAGGTCTAGATAGAGGCGAGCGTAATCTGATTTCCTACG | |
| | TAGTATTGAACCGAGAAGGCAAGATTGTTAACAATCAGCAGGGGAGTTTCAAT | |
| | ATTGTGGGTAAGATGGACTATCAGAAGAAACTGTATCAAAAAGAAAAGAATCG | |
| | TGACAAAGAACGAAAAACTTGGAAAAATATCGAAACAATAAAGGATTTGAAGG | |
| | AAGGATATATTTCACAAGTCGTTCATGAATTGACCGATATGGCGATTCGCAAT | |
| | AATGCAATTATTGTGATGGAAGATCTGAATTTTGGATTTAAAAGGGGACGCAC | |
| | CAAAGTGGAACGGCAGGTATATCAGAAGTTTGAGCTGGCGCTTCTGAAGAAA | |
| | TTGCATTATCTGGTTACGGATAAAACAGAAGGTGAGGCTATGCTTAAGCCTGG | |
| | CGGTGTCCTTCAAGGTTATCAGCTTGCAAGAGAAGTAAAAACCCTAAAAGAAA | |
| | TCGGAAAGCAATGCGGATGTGTATTTTATGTTCCACCGGGATATACTTCTAAA | |
| | ATCGATCCAACAACCGGATTTGTTGATGTGTTTAACATGTCAGGTGTTACGAA | |
| | TCGTGAAAAGAAAAAAGCATTTTTTGAAAAGTTCGATAATATGTTCTATGATGA | |
| | AAAGCGGGATATGTTTGGATTTTCATTTAACTATGAGAAGTTTACAACATATCA | |
| | AAGTTCTTATAGAAATGATTGGACTGTATATTCGAATGGAAGCAAATATGTGTG | |
| | GAACTCTTTAAAAAGGACAGACGAGCTTATTGATGTTACAAAAGAATTGAAACT | |
| | GCTCTTTGAAAAGTATGCAATTGATTACAGAAACGAAGCATTGTTTGAACAAAT | |
| | CATGTCCCAAGATACGGATAAAAACAATGCTGACTTTTGGAATAAATTGTTCTG | |
| | GTATTTTCGTGTTTTGCTCCGTCTGAGAAACAGTTCAGATGAATTAGATCAGAT | |
| | TGTTTCACCGGTACTTAATCAAAACGGAGAATTTTTTGAAACACCGAAAAAAAT | |
| | CACGGAGAAAAGTTATTTGTCTGATTATCCGATGGATGCGGATACCAATGGTG | |
| | CGTATCACATCGCTTTAAAAGGGTTGTATCTCATACAGGAAAAAATTGCAGAT | |
| | GAGAGCGTAGATTTGGATAACAAATTACCAAAAGATTTTTACAAGATCTCTAAT | |
| | GCAGAGTGGTTTATGTTTAGGCAGAAGGAGAAGTAA | |
| Codon | AAGAGCATCTACGAGAACTTCATCGGTCTTGAGAGCAAGAACCTGACACTGA | 11 |
| optimized | GATTCGCCCTGAACCCTGAGGCTAAAACCCAGGAGAACCTGAAGCTGTACTG | |
| coding | GGACAAACTGAGGGACGAAGAAAGAGATAGAGCCTACCCTATCGTGAAAAAA | |
| sequence (no | ATCCTCGACAAGGAGTATCAGCAGCTCATCAGCGAGGGCCTGAAACTGCTGG | |
| N-terminal | AAAATCAAAACGTGCTGGACTGGACCGAACTGGCCGAGTACATCAGAACCAG | |
| methionine, no | CGATCTGTCTAAGAAGAAGAAGGAGGACAAGAGACTGCGCAAGCTGATCGCC | |
| stop codon) | CAGAACCTGAAAGCCCACCCCCTGGTCGACAAGCTGAAGGTGAAGAATGCCT | |
| | TCGGCAAGAACGGCTACCTGGAAACCCTGCCATTAGGAAAGGAAGAAAAAGA | |
| | GGCCGTGAAGGTGTTTGCCGGATTCGGAGGCTTTTTCAACAACTACAACAAG | |
| | AATCGGGAGAACTACTTCAGTACCGAGGAGAAGTCCACCGCCATCGCCAACA | |
| | GAATCGTGAACGAGAACTTCAGCAAGCACTTCAGCAACGTGGAAATCGTTACA | |
| | AAGATCCAAAAAGAAGTGCCAGAGCTGATTCAAATCGTGGAAGCTCAGTTCAA | |
| | GGGTTACGACACCATCTTTACCGTGAACGGCTACAACACCGCCCTGAGCCAG | |
| | GCTGGCATCGACACATACAACGAAATGGTGGCCATCTGGAACAAGGAGGCAA | |
| | ACCTGTACGCTCAAAAAGCCGGCAAGCTGCCAGACGGCCACCCGCTGAAGA | |
| | AGAAGCGTAACTACCTGCTGAGCGCCCTCTTCAAACAGATCGGCAGCGAAAA | |
| | AGAACACCTGATCCAGATCGACAGATTCGACGGCGACGAGGAAGTGATCGAA | |
| | GCCCTGACTGGCGTGAAAAAGATGCTGCAGGAGGCCGACGTGTTCGAGAAG | |
| | CTGAACATGCTGGTCGAGGACATGGAAAATTGGGATTACTCCAAGATCTACCT | |
| | GTCTGCCCAGAGCCTGAGTAACGTGTCCGTGTTCCTGAACAACCTGTATGAA | |
| | GATGAACGGGAGAACAGCTGGAACTACCTGGATAACGTGCTGAGAGAGAAGT | |
| | GGCAGATTGAACTGCAGGGCAAAAAAAAGGGAACAGATCTGGAAGAGGCCAT | |
| | TAGAAAGAAGAAGCAGAGCTTTTACTCTATCGAGGAACTTCAGGAGGCAGTG | |
| | AACGCCATCGAGGAAACCGACAAGTGCTACAATGTGTCTAAATGGCTGCTGG | |
| | GAGCCATGAAGAGCGAGAGAGTGATCGAGGAGAAGAAGAAAGACGTGGAGG | |
| | ATTTCTGCACACAGTGGAAGAACGAGAGAAACAGCCTCAAGGAAACCGACAT | |
| | CACCGCCCTGAAGGAGTACCTGGAGCAGTGGATCTTCCTGGCTAGGTACTGC | |
| | AAGAGCTTCTACGCCAATGGCATCGAAAAGAAAGAGAAGGATGAGGCTTTTTA | |
| | CCACATCCTGGAGGATGTGCTGTACGTGCTGGACGAAGTGATCTACTTCTAC | |
| | AACAAGGTGCGGAACTACGTGACCAAAAAGCCTTACAGTCTGGAGAAGATGC | |
| | ACCTGAAGTTCGGCCACAACGAGCTGGCCAACGGCTGGAGCGTGAACAAGG | |
| | AAGAAAATTACGGCACCGCCATCCTGAGAAGAAACGGCAAGTACTACCTGGC | |
| | CATCACCAACAGCCTGAACAAGAAAATGAGCATCCCTACCCAGCTGGAGAGC | |
| | ACAGGAAATAATTATGAGAAGATGGTCCTGAACGTTTTTCCCAACGTGTTCCG | |
| | GATGATCCCAAAGTGCACCACAGGCAGGAACGACGTGAAGTCATGCTTCGAG | |
| | AGAAAGGAACCCAACGAGTACTTCTTCATCGACACCCCTGAGTTCGTGAACC | |
| | CCTTTAAGGTCACACGGGAGGAGTACGAACTGAATAAGATCACCTACGACGG | |
| | AGTTAAGAAGTGGCAGAGCGACTACAGCAAGAACACACAGGACGAAAAGGGC | |
| | TATAAGGAAGCCGTGACCAAGTGGATTCAGTTTTGTATGCGGTTCCTGCAGTC | |
| | TTATAAGAGCACCGCCATATATGACTACAGCACCCTGCAGCAACCTGAAAAAT | |
| | ACGAAACAGTGGACAGCTTCTATCATGATGTGGAAAAGATCCTGTACGAGTGC | |
| | CACTTCGAGTACGTGCCCGCTAACAAGATCGAGCAGCTTGAAGAAGAGGGAA | |
| | GAATCTTCCTGTTCCAGATCTACAACAAGGATTTTTCTGAGAACAGACGGCCT | |
| | GATAGCAAGAAAAACCTCCACACCCTGTACTGGGAGGCGCTGTTCTCCGAAG | |
| | AGAATAGAAAGGCCAAGGTGATTCAGCTGAATGGCAAGGCCGAGATCTTCAG | |
| | ACGGGAGAAATCAATCGAGCACCCTATCGTGCATAAGGCTGGCGAGGTGCTG | |
| | GTGAACAAGCGGACCAAAGATGGCGAACCTATTCCTGACGACATCTACAAGG | |
| | ACCTGAGCAACTATTTCAACGGCAGAAACGTTACCTCTGAGAAGGAAGAGTAC | |
| | AAGGAGTGTCTGGACAAGGTGTACACCAGCACCAAAAAGTACGATATCACCA | |
| | AGGACAAAAGATTCACCGAGACAAAGTACGAGTTCCACGTGCCTATCACCCT | |
| | GAACTACCAGGCCGACGGCGTGAAGTACCTGAATCAGAAGATCCTGCACGTG | |
| | CTGCGGGACAACCCTGATGTTAACATCATCGGCCTGGATAGAGGCGAAAGAA | |
| | ACCTGATCTCTTATGTTGTGCTGAACAGAGAGGGCAAGATCGTGAACAATCAG | |
| | CAGGGTTCTTTCAACATCGTGGGCAAAATGGACTACCAGAAAAAGCTGTACCA | |
| | GAAGGAGAAAAACCGGGATAAAGAACGGAAAACGTGGAAAAACATCGAAACC | |
| | ATCAAGGACCTGAAGGAGGGCTATATCAGCCAGGTGGTACACGAGCTGACCG | |
| | ATATGGCCATCCGGAATAACGCGATCATCGTGATGGAAGATCTGAATTTCGGA | |
| | TTCAAGCGGGGCCGGACCAAGGTGGAACGGCAGGTGTACCAGAAGTTTGAG | |
| | CTGGCCCTGCTGAAGAAGCTGCACTACCTCGTGACCGACAAGACCGAGGGA | |
| | GAAGCTATGCTGAAACCCGGCGGCGTGCTGCAAGGCTACCAGCTGGCTAGA | |
| | GAAGTCAAGACCCTGAAAGAGATCGGCAAGCAGTGCGGCTGTGTGTTCTACG | |
| | TGCCCCCTGGCTACACAAGCAAGATCGACCCTACAACCGGCTTCGTCGACGT | |
| | GTTCAACATGTCTGGAGTTACAAACCGCGAGAAAAAGAAAGCCTTTTTCGAAA | |
| | AATTTGATAACATGTTCTACGACGAGAAGAGAGACATGTTCGGCTTCAGCTTC | |
| | AATTACGAAAAGTTTACTACCTACCAGAGCAGCTACAGAAACGACTGGACCGT | |
| | GTACAGCAACGGCAGCAAGTATGTGTGGAACTCCCTTAAGAGAACAGACGAG | |
| | TTAATTGACGTGACAAAGGAGCTCAAGCTGCTGTTCGAGAAGTACGCCATCG | |
| | ATTACCGGAACGAAGCTCTGTTTGAGCAGATCATGAGCCAGGATACAGATAA | |
| | GAACAACGCCGACTTCTGGAACAAACTGTTCTGGTACTTCCGGGTGCTGCTG | |
| | CGGCTGAGAAATAGCAGCGACGAACTGGACCAAATCGTCAGCCCTGTGCTGA | |
| | ATCAGAACGGAGAGTTCTTCGAAACCCCTAAGAAAATCACAGAGAAGTCCTAC | |
| | CTGTCTGATTACCCTATGGACGCCGATACAAACGGCGCCTACCACATCGCCC | |
| | TGAAGGGCCTGTACCTGATCCAGGAGAAGATCGCTGACGAATCTGTGGACCT | |
| | GGACAACAAGCTGCCTAAGGACTTCTACAAGATCAGCAACGCCGAGTGGTTC | |
| | ATGTTTAGACAGAAAGAAAAA | |
| Expression | ATGggcAAGAGCATCTACGAGAACTTCATCGGTCTTGAGAGCAAGAACCTGAC | 12 |
| construct (with | ACTGAGATTCGCCCTGAACCCTGAGGCTAAAACCCAGGAGAACCTGAAGCTG | |
| N-terminal | TACTGGGACAAACTGAGGGACGAAGAAAGAGATAGAGCCTACCCTATCGTGA | |
| methionine | AAAAAATCCTCGACAAGGAGTATCAGCAGCTCATCAGCGAGGGCCTGAAACT | |
| and stop | GCTGGAAAATCAAAACGTGCTGGACTGGACCGAACTGGCCGAGTACATCAGA | |
| codon, | ACCAGCGATCTGTCTAAGAAGAAGAAGGAGGACAAGAGACTGCGCAAGCTGA | |
| includes V5- | TCGCCCAGAACCTGAAAGCCCACCCCCTGGTCGACAAGCTGAAGGTGAAGAA | |
| tag and C- | TGCCTTCGGCAAGAACGGCTACCTGGAAACCCTGCCATTAGGAAAGGAAGAA | |
| terminal NLS) | AAAGAGGCCGTGAAGGTGTTTGCCGGATTCGGAGGCTTTTTCAACAACTACA | |
| | ACAAGAATCGGGAGAACTACTTCAGTACCGAGGAGAAGTCCACCGCCATCGC | |
| | CAACAGAATCGTGAACGAGAACTTCAGCAAGCACTTCAGCAACGTGGAAATC | |
| | GTTACAAAGATCCAAAAAGAAGTGCCAGAGCTGATTCAAATCGTGGAAGCTCA | |
| | GTTCAAGGGTTACGACACCATCTTTACCGTGAACGGCTACAACACCGCCCTG | |
| | AGCCAGGCTGGCATCGACACATACAACGAAATGGTGGCCATCTGGAACAAGG | |
| | AGGCAAACCTGTACGCTCAAAAAGCCGGCAAGCTGCCAGACGGCCACCCGC | |
| | TGAAGAAGAAGCGTAACTACCTGCTGAGCGCCCTCTTCAAACAGATCGGCAG | |
| | CGAAAAAGAACACCTGATCCAGATCGACAGATTCGACGGCGACGAGGAAGTG | |
| | ATCGAAGCCCTGACTGGCGTGAAAAAGATGCTGCAGGAGGCCGACGTGTTC | |
| | GAGAAGCTGAACATGCTGGTCGAGGACATGGAAAATTGGGATTACTCCAAGA | |
| | TCTACCTGTCTGCCCAGAGCCTGAGTAACGTGTCCGTGTTCCTGAACAACCT | |
| | GTATGAAGATGAACGGGAGAACAGCTGGAACTACCTGGATAACGTGCTGAGA | |
| | GAGAAGTGGCAGATTGAACTGCAGGGCAAAAAAAAGGGAACAGATCTGGAAG | |
| | AGGCCATTAGAAAGAAGAAGCAGAGCTTTTACTCTATCGAGGAACTTCAGGAG | |
| | GCAGTGAACGCCATCGAGGAAACCGACAAGTGCTACAATGTGTCTAAATGGC | |
| | TGCTGGGAGCCATGAAGAGCGAGAGAGTGATCGAGGAGAAGAAGAAAGACG | |
| | TGGAGGATTTCTGCACACAGTGGAAGAACGAGAGAAACAGCCTCAAGGAAAC | |
| | CGACATCACCGCCCTGAAGGAGTACCTGGAGCAGTGGATCTTCCTGGCTAGG | |
| | TACTGCAAGAGCTTCTACGCCAATGGCATCGAAAAGAAAGAGAAGGATGAGG | |
| | CTTTTTACCACATCCTGGAGGATGTGCTGTACGTGCTGGACGAAGTGATCTAC | |
| | TTCTACAACAAGGTGCGGAACTACGTGACCAAAAAGCCTTACAGTCTGGAGAA | |
| | GATGCACCTGAAGTTCGGCCACAACGAGCTGGCCAACGGCTGGAGCGTGAA | |
| | CAAGGAAGAAAATTACGGCACCGCCATCCTGAGAAGAAACGGCAAGTACTAC | |
| | CTGGCCATCACCAACAGCCTGAACAAGAAAATGAGCATCCCTACCCAGCTGG | |
| | AGAGCACAGGAAATAATTATGAGAAGATGGTCCTGAACGTTTTTCCCAACGTG | |
| | TTCCGGATGATCCCAAAGTGCACCACAGGCAGGAACGACGTGAAGTCATGCT | |
| | TCGAGAGAAAGGAACCCAACGAGTACTTCTTCATCGACACCCCTGAGTTCGT | |
| | GAACCCCTTTAAGGTCACACGGGAGGAGTACGAACTGAATAAGATCACCTAC | |
| | GACGGAGTTAAGAAGTGGCAGAGCGACTACAGCAAGAACACACAGGACGAAA | |
| | AGGGCTATAAGGAAGCCGTGACCAAGTGGATTCAGTTTTGTATGCGGTTCCT | |
| | GCAGTCTTATAAGAGCACCGCCATATATGACTACAGCACCCTGCAGCAACCT | |
| | GAAAAATACGAAACAGTGGACAGCTTCTATCATGATGTGGAAAAGATCCTGTA | |
| | CGAGTGCCACTTCGAGTACGTGCCCGCTAACAAGATCGAGCAGCTTGAAGAA | |
| | GAGGGAAGAATCTTCCTGTTCCAGATCTACAACAAGGATTTTTCTGAGAACAG | |
| | ACGGCCTGATAGCAAGAAAAACCTCCACACCCTGTACTGGGAGGCGCTGTTC | |
| | TCCGAAGAGAATAGAAAGGCCAAGGTGATTCAGCTGAATGGCAAGGCCGAGA | |
| | TCTTCAGACGGGAGAAATCAATCGAGCACCCTATCGTGCATAAGGCTGGCGA | |
| | GGTGCTGGTGAACAAGCGGACCAAAGATGGCGAACCTATTCCTGACGACATC | |
| | TACAAGGACCTGAGCAACTATTTCAACGGCAGAAACGTTACCTCTGAGAAGGA | |
| | AGAGTACAAGGAGTGTCTGGACAAGGTGTACACCAGCACCAAAAAGTACGAT | |
| | ATCACCAAGGACAAAAGATTCACCGAGACAAAGTACGAGTTCCACGTGCCTAT | |
| | CACCCTGAACTACCAGGCCGACGGCGTGAAGTACCTGAATCAGAAGATCCTG | |
| | CACGTGCTGCGGGACAACCCTGATGTTAACATCATCGGCCTGGATAGAGGCG | |
| | AAAGAAACCTGATCTCTTATGTTGTGCTGAACAGAGAGGGCAAGATCGTGAAC | |
| | AATCAGCAGGGTTCTTTCAACATCGTGGGCAAAATGGACTACCAGAAAAAGCT | |
| | GTACCAGAAGGAGAAAAACCGGGATAAAGAACGGAAAACGTGGAAAAACATC | |
| | GAAACCATCAAGGACCTGAAGGAGGGCTATATCAGCCAGGTGGTACACGAGC | |
| | TGACCGATATGGCCATCCGGAATAACGCGATCATCGTGATGGAAGATCTGAA | |
| | TTTCGGATTCAAGCGGGGCCGGACCAAGGTGGAACGGCAGGTGTACCAGAA | |
| | GTTTGAGCTGGCCCTGCTGAAGAAGCTGCACTACCTCGTGACCGACAAGACC | |
| | GAGGGAGAAGCTATGCTGAAACCCGGCGGCGTGCTGCAAGGCTACCAGCTG | |
| | GCTAGAGAAGTCAAGACCCTGAAAGAGATCGGCAAGCAGTGCGGCTGTGTGT | |
| | TCTACGTGCCCCCTGGCTACACAAGCAAGATCGACCCTACAACCGGCTTCGT | |
| | CGACGTGTTCAACATGTCTGGAGTTACAAACCGCGAGAAAAAGAAAGCCTTTT | |
| | TCGAAAAATTTGATAACATGTTCTACGACGAGAAGAGAGACATGTTCGGCTTC | |
| | AGCTTCAATTACGAAAAGTTTACTACCTACCAGAGCAGCTACAGAAACGACTG | |
| | GACCGTGTACAGCAACGGCAGCAAGTATGTGTGGAACTCCCTTAAGAGAACA | |
| | GACGAGTTAATTGACGTGACAAAGGAGCTCAAGCTGCTGTTCGAGAAGTACG | |
| | CCATCGATTACCGGAACGAAGCTCTGTTTGAGCAGATCATGAGCCAGGATAC | |
| | AGATAAGAACAACGCCGACTTCTGGAACAAACTGTTCTGGTACTTCCGGGTG | |
| | CTGCTGCGGCTGAGAAATAGCAGCGACGAACTGGACCAAATCGTCAGCCCTG | |
| | TGCTGAATCAGAACGGAGAGTTCTTCGAAACCCCTAAGAAAATCACAGAGAAG | |
| | TCCTACCTGTCTGATTACCCTATGGACGCCGATACAAACGGCGCCTACCACAT | |
| | CGCCCTGAAGGGCCTGTACCTGATCCAGGAGAAGATCGCTGACGAATCTGTG | |
| | GACCTGGACAACAAGCTGCCTAAGGACTTCTACAAGATCAGCAACGCCGAGT | |
| | GGTTCATGTTTAGACAGAAAGAAAAAtctagaAAGCGGACAGCAGACGGCTCCG | |
| | AATTTGAAAGCCCTAAGAAAAAGAGAAAGGTGggatccGGCAAACCTATCCCCA | |
| | ATCCCCTGCTGGGCCTGGACAGCACCTGA | |

In some embodiments a ZJHK Type V Cas protein comprises an amino acid sequence of SEQ ID NO: 7, SEQ ID NO:8, or SEQ ID NO:9. In some embodiments, a ZJHK Type V Cas protein has nickase activity, for example resulting from one or more amino acid substitutions relative to the sequence of SEQ ID NO: 7, SEQ ID NO:8, or SEQ ID NO:9. In some embodiments, the one or more amino acid substitutions providing nickase activity comprise a D900 substitution, wherein the position of the D900 substitution is defined with respect to the amino acid numbering of SEQ ID NO:8 (corresponding to amino acid 908 of SEQ ID NO: 121). In some embodiments, the one or more amino acid substitutions providing nickase activity comprise an E987 substitution, wherein the position of the E987 substitution is defined with respect to the amino acid numbering of SEQ ID NO:8 (corresponding to amino acid 993 of SEQ ID NO: 121). In some embodiments, the one or more amino acid substitutions providing nickase activity comprise a R1203 substitution, wherein the position of the R1203 substitution is defined with respect to the amino acid numbering of SEQ ID NO:8 (corresponding to amino acid 1226 of SEQ ID NO:121). In some embodiments, the one or more amino acid substitutions providing nickase activity comprise a D1244 substitution, wherein the position of the D1244 substitution is defined with respect to the amino acid numbering of SEQ ID NO: 121 (corresponding to amino acid 1263 of SEQ ID NO: 121). In some embodiments, a ZJHK Type V Cas protein is catalytically inactive, for example due to a R1203 substitution in combination with a D900 substitution, a E987 substitution, and/or D1244 substitution.

### 6.2.3. ZIKV Type V Cas Proteins

In one aspect, the disclosure provides ZIKV Type V Cas proteins. ZIKV Type V Cas proteins can be further classified as Type V-A Cas proteins. The ZIKV Type V Cas proteins typically comprise an amino acid sequence that is at least 50%, at least 55%, at least 60%, at least 65%, at least 70%, at least 75%, at least 85%, at least 90%, or at least 95% identical to SEQ ID NO: 13. In some embodiments, the ZIKV Type V Cas proteins comprise an amino acid sequence that is at least 95%, at least 96%, at least 97%, at least 98%, or at least 99% identical to SEQ ID NO: 13. In some embodiments, a ZIKV Type V Cas protein comprises an amino acid sequence that is identical to SEQ ID NO: 13.

Exemplary ZIKV Type V Cas protein sequences and nucleotide sequences encoding exemplary ACEE Type V Cas proteins are set forth in Table 1C.

TABLE 1C
ZIKV Type V Cas Sequences

| | | SEQ ID |
|---|---|---|
| Name | Sequence | NO. |
| Wildtype | NIYENFTNMYQVNKTIRMGLKPICKTGENIAKFLEEDKETSDKYKIAKEVIDKENRA | 13 |
| amino acid | FIEDRLKDFSISGLDEYLELLKQKKNLTKNQNKMKKEISTQLTKIQNKMRDEISTQL | |
| sequence | KGFPQFDNKYKFKYITDKEDIEILKYFKDKKFITFFEEFNTNRKNVYSKENISTSIGH | |
| (without N- | RIVHENLPKFISNFRILNKAIEAFGISKINEDFKNNGINVTVEELNKIDYFNKVLTQSG | |
| terminal | IDLYNNLIGILNQNINLYNQQQKVKKNKIGKLEILYKQILSKTDKVSFIEEFTEDNQLL | |
| methionine) | ECIDEYFKEKYSLITVDLNNLLENIDTYNLNGIFIKSDKSLGNISNYLYKDWWYISNLI | |
| | NEEYDYKHKNKVRDDKYYETRKKAIDKIKYFSIGHIDELLKDKNVPMVENYFKEKIN | |
| | LVVKEFNAYLNKFNEYKFINELKTDEIAVEIIKNLCDSIKNVQGIVKPLIITGNDKDDD | |
| | FYVEINYIWDELNKFDKIYNMVRNYLTKKDYIEEKIRMMFSKSSFMDGWGKDYGT | |
| | KKAHIVYHDKNYYLVIVDKKLKLEDIDKLYKPGGDTVHYVYNYQSTENGNIPRKFIY | |
| | SKGKRFAPSVEKYNLPIEDVIEVYNNEYHTTDYEKKNPEIYKKSLTSLIDYFKIGVN | |
| | RDMDFEKFDFRLKDSNEYKNIKEFYDNLETCCYKLQEEKVNFNVLEELSYSGKIYL | |
| | FKIYNKDFSENSKGIPNLHTLYFKMLFDKENLENPIYKLSGKAKMFFRKGSLNLDK | |
| | KTVDYDKKPIDKKENDKKIKNRRYKVDSFTLHMSIITNFQSYENKNVNETVNRALK | |
| | YCDDVYAIGIDRGIRNLLYACVVNSKGEIVKQVPLNIINNKDYHNLLAEREEKKKNS | |
| | RKNWKIIDNIRNLKEGYLSQAIHIITDLMVEYNAVLVLENLNFRFKEKQMKFESNVY | |
| | QKFEKMLIDKLNFLVDKKLDKNANGGLFNAYQLTEKFTNFKDMKNQNGIIFYIPAW | |
| | MTSKIDPVTGFTNLFYIKYESIEKAKEFFGKFKSIKFNKVDNYFEFEFDYNDFTDRA | |
| | QGTRSKWTVCSFGPRIEGFRNPEKNNSWDGREIDITEKIKKLLDDYNVSLDKDIKA | |
| | QIMDINTKDFFEKFIKYFKLVLQMRNSKTGTDIDYIISPVKNKQNEFFDSRKQNEKM | |
| | PMDADANGAYNIARKGLMFIDIIKETEDKDLKMPKLFIKNKDWLNYVQKSDL | |
| Wildtype | MNIYENFTNMYQVNKTIRMGLKPICKTGENIAKFLEEDKETSDKYKIAKEVIDKENR | 14 |
| amino acid | AFIEDRLKDFSISGLDEYLELLKQKKNLTKNQNKMKKEISTQLTKIQNKMRDEISTQ | |
| sequence (with | LKGFPQFDNKYKFKYITDKEDIEILKYFKDKKFITFFEEFNTNRKNVYSKENISTSIG | |
| N-terminal | HRIVHENLPKFISNFRILNKAIEAFGISKINEDFKNNGINVTVEELNKIDYFNKVLTQS | |
| methionine) | GIDLYNNLIGILNQNINLYNQQQKVKKNKIGKLEILYKQILSKTDKVSFIEEFTEDNQL | |
| | LECIDEYFKEKYSLITVDLNNLLENIDTYNLNGIFIKSDKSLGNISNYLYKDWWYISN | |
| | LINEEYDYKHKNKVRDDKYYETRKKAIDKIKYFSIGHIDELLKDKNVPMVENYFKEK | |
| | INLVVKEFNAYLNKFNEYKFINELKTDEIAVEIIKNLCDSIKNVQGIVKPLIITGNDKDD | |
| | DFYVEINYIWDELNKFDKIYNMVRNYLTKKDYIEEKIRMMFSKSSFMDGWGKDYG | |
| | TKKAHIVYHDKNYYLVIVDKKLKLEDIDKLYKPGGDTVHYVYNYQSTENGNIPRKFI | |
| | YSKGKRFAPSVEKYNLPIEDVIEVYNNEYHTTDYEKKNPEIYKKSLTSLIDYFKIGV | |
| | NRDMDFEKFDFRLKDSNEYKNIKEFYDNLETCCYKLQEEKVNFNVLEELSYSGKI | |
| | YLFKIYNKDFSENSKGIPNLHTLYFKMLFDKENLENPIYKLSGKAKMFFRKGSLNL | |
| | DKKTVDYDKKPIDKKENDKKIKNRRYKVDSFTLHMSIITNFQSYENKNVNETVNRA | |
| | LKYCDDVYAIGIDRGIRNLLYACVVNSKGEIVKQVPLNIINNKDYHNLLAEREEKKK | |
| | NSRKNWKIIDNIRNLKEGYLSQAIHIITDLMVEYNAVLVLENLNFRFKEKQMKFESN | |
| | VYQKFEKMLIDKLNFLVDKKLDKNANGGLFNAYQLTEKFTNFKDMKNQNGIIFYIP | |
| | AWMTSKIDPVTGFTNLFYIKYESIEKAKEFFGKFKSIKFNKVDNYFEFEFDYNDFTD | |
| | RAQGTRSKWTVCSFGPRIEGFRNPEKNNSWDGREIDITEKIKKLLDDYNVSLDKDI | |
| | KAQIMDINTKDFFEKFIKYFKLVLQMRNSKTGTDIDYIISPVKNKQNEFFDSRKQNE | |
| | KMPMDADANGAYNIARKGLMFIDIIKETEDKDLKMPKLFIKNKDWLNYVQKSDL | |
| Expression | MGNIYENFTNMYQVNKTIRMGLKPICKTGENIAKFLEEDKETSDKYKIAKEVIDKEN | 15 |
| construct (with | RAFIEDRLKDFSISGLDEYLELLKQKKNLTKNQNKMKKEISTQLTKIQNKMRDEIST | |
| N-terminal | QLKGFPQFDNKYKFKYITDKEDIEILKYFKDKKFITFFEEFNTNRKNVYSKENISTSI | |
| methionine, | GHRIVHENLPKFISNFRILNKAIEAFGISKINEDFKNNGINVTVEELNKIDYFNKVLTQ | |
| V5-tag and C- | SGIDLYNNLIGILNQNINLYNQQQKVKKNKIGKLEILYKQILSKTDKVSFIEEFTEDN | |
| terminal NLS) | QLLECIDEYFKEKYSLITVDLNNLLENIDTYNLNGIFIKSDKSLGNISNYLYKDWWYI | |
| aa sequence | SNLINEEYDYKHKNKVRDDKYYETRKKAIDKIKYFSIGHIDELLKDKNVPMVENYFK | |
| | EKINLVVKEFNAYLNKFNEYKFINELKTDEIAVEIIKNLCDSIKNVQGIVKPLIITGNDK | |
| | DDDFYVEINYIWDELNKFDKIYNMVRNYLTKKDYIEEKIRMMFSKSSFMDGWGKD | |
| | YGTKKAHIVYHDKNYYLVIVDKKLKLEDIDKLYKPGGDTVHYVYNYQSTENGNIPR | |
| | KFIYSKGKRFAPSVEKYNLPIEDVIEVYNNEYHTTDYEKKNPEIYKKSLTSLIDYFKI | |
| | GVNRDMDFEKFDFRLKDSNEYKNIKEFYDNLETCCYKLQEEKVNFNVLEELSYSG | |
| | KIYLFKIYNKDFSENSKGIPNLHTLYFKMLFDKENLENPIYKLSGKAKMFFRKGSLN | |
| | LDKKTVDYDKKPIDKKENDKKIKNRRYKVDSFTLHMSIITNFQSYENKNVNETVNR | |
| | ALKYCDDVYAIGIDRGIRNLLYACVVNSKGEIVKQVPLNIINNKDYHNLLAEREEKK | |
| | KNSRKNWKIIDNIRNLKEGYLSQAIHIITDLMVEYNAVLVLENLNFRFKEKQMKFES | |
| | NVYQKFEKMLIDKLNFLVDKKLDKNANGGLFNAYQLTEKFTNFKDMKNQNGIIFYI | |
| | PAWMTSKIDPVTGFTNLFYIKYESIEKAKEFFGKFKSIKFNKVDNYFEFEFDYNDFT | |
| | DRAQGTRSKWTVCSFGPRIEGFRNPEKNNSWDGREIDITEKIKKLLDDYNVSLDK | |
| | DIKAQIMDINTKDFFEKFIKYFKLVLQMRNSKTGTDIDYIISPVKNKQNEFFDSRKQ | |
| | NEKMPMDADANGAYNIARKGLMFIDIIKETEDKDLKMPKLFIKNKDWLNYVQKSDL | |
| | SRKRTADGSEFESPKKKRKVGSGKPIPNPLLGLDST | |
| Wildtype | ATGAACATTTACGAAAATTTTACTAATATGTATCAGGTAAATAAGACTATAAGAA | 16 |
| coding | TGGGGTTAAAGCCAATATGTAAAACTGGTGAAAATATTGCTAAATTTCTTGAGG | |
| sequence (with | AAGATAAGGAAACAAGTGATAAATACAAGATAGCTAAAGAAGTAATTGATAAG | |
| N-terminal | GAAAATAGAGCTTTTATAGAGGATAGATTAAAGGATTTTTCAATTTCAGGGTTG | |
| methionine | GATGAATATTTGGAATTGCTTAAACAAAAAAAGAATTTAACCAAAAATCAAAAT | |
| and stop | AAAATGAAAAAGGAAATTTCAACACAGTTAACAAAAATACAAAATAAAATGAGA | |
| codon) | GATGAAATTTCAACACAGTTAAAAGGCTTCCCTCAATTTGATAATAAATATAAA | |
| | TTCAAATATATTACAGATAAAGAAGATATAGAAATTTTAAAATATTTTAAAGATA | |
| | AGAAATTTATTACTTTCTTTGAAGAATTTAATACTAATAGAAAAAATGTCTACTC | |
| | TAAAGAAAATATTTCAACTTCTATTGGACACAGAATTGTTCACGAAAATCTTCC | |
| | AAAATTTATTTCAAATTTTAGGATTTTAAATAAAGCAATAGAGGCGTTTGGAATA | |
| | AGTAAAATAAATGAAGATTTTAAGAATAATGGAATTAATGTTACAGTTGAAGAA | |
| | CTTAATAAAATAGATTATTTTAACAAGGTTTTAACTCAATCAGGAATAGATTTGT | |
| | ATAATAATTTGATAGGTATTTTAAATCAAAATATAAATCTATATAATCAACAACA | |
| | GAAAGTAAAAAAGAATAAAATTGGAAAGTTAGAAATATTATATAAGCAAATTTTA | |
| | AGTAAAACAGATAAAGTATCGTTTATTGAAGAATTTACTGAAGATAACCAACTT | |
| | TTGGAATGTATTGATGAATATTTTAAAGAAAAATATAGTTTGATAACTGTAGATT | |
| | TAAATAATTTACTTGAAAATATTGATACTTATAATTTGAATGGTATCTTTATTAAA | |
| | AGTGATAAGTCCTTGGGAAATATATCTAATTATTTATATAAAGATTGGTGGTAT | |
| | ATATCAAATCTTATAAACGAAGAATACGATTATAAACATAAGAATAAGGTAAGA | |
| | GATGATAAGTATTATGAAACAAGAAAAAAAGCTATAGATAAGATTAAATATTTTT | |
| | CCATAGGACATATTGATGAATTGTTAAAAGATAAAAATGTTCCTATGGTAGAAA | |
| | ACTATTTCAAAGAAAAGATAAATTTAGTAGTAAAAGAATTTAATGCTTATTTAAA | |
| | CAAATTTAATGAATATAAGTTTATAAATGAGCTAAAAACTGATGAAATTGCTGT | |
| | CGAAATAATAAAAAATTTATGTGATTCAATAAAGAATGTACAGGGGATAGTAAA | |
| | GCCTTTAATAATTACTGGAAATGATAAAGACGATGATTTTTATGTGGAAATCAA | |
| | TTATATATGGGACGAGCTTAATAAGTTTGATAAAATATATAATATGGTTAGAAAT | |
| | TATCTTACAAAAAAGGATTACATAGAGGAAAAAATTAGAATGATGTTTTCAAAG | |
| | AGCAGTTTTATGGATGGTTGGGGAAAAGATTATGGAACAAAAAAAGCACATAT | |
| | AGTTTATCATGATAAAAATTATTATTTAGTAATAGTAGACAAGAAATTAAAATTA | |
| | GAGGATATAGATAAATTATATAAACCAGGTGGAGATACTGTACATTATGTATAT | |
| | AATTACCAATCAACAGAAAATGGAAATATTCCTAGAAAATTCATATATTCTAAG | |
| | GGTAAAAGATTTGCACCATCTGTAGAAAAATATAATTTACCAATAGAAGATGTT | |
| | ATCGAAGTGTATAACAATGAATATCATACAACAGATTACGAAAAGAAAAATCCT | |
| | GAAATTTACAAGAAATCATTAACATCCTTAATTGATTATTTTAAAATAGGGGTAA | |
| | ATAGGGATATGGATTTTGAAAAATTTGATTTTAGATTAAAAGATTCAAACGAAT | |
| | ACAAAAATATAAAAGAATTTTATGATAATTTGGAAACTTGTTGCTATAAGTTACA | |
| | AGAAGAAAAAGTTAATTTTAATGTACTTGAAGAGCTTTCATATAGTGGAAAAAT | |
| | TTATTTATTTAAAATATACAATAAGGATTTTTCTGAAAATAGCAAAGGAATACCT | |
| | AATCTTCATACTTTATATTTTAAAATGCTATTTGACAAAGAAAACCTTGAAAATC | |
| | CGATTTATAAACTTAGTGGAAAGGCTAAAATGTTTTTTAGAAAGGGTAGTCTTA | |
| | ATTTAGACAAAAAAACTGTTGATTATGATAAAAAGCCAATAGATAAGAAAGAAA | |
| | ATGACAAAAAAATTAAAAATAGAAGATATAAAGTTGATAGTTTTACATTACATAT | |
| | GTCAATTATTACGAACTTTCAGTCATATGAAAATAAAAATGTAAATGAAACTGT | |
| | AAATAGGGCTTTAAAATATTGTGATGATGTTTATGCCATAGGTATAGACAGAG | |
| | GAATAAGAAATTTATTATATGCTTGTGTAGTAAATTCAAAGGGAGAAATAGTAA | |
| | AACAAGTTCCTTTAAATATTATAAATAATAAAGATTATCACAATTTACTTGCAGA | |
| | AAGAGAAGAGAAGAAAAAGAATAGTAGGAAAAATTGGAAAATCATTGATAATA | |
| | TAAGGAATTTAAAGGAAGGCTATTTAAGTCAGGCCATACATATAATAACTGACC | |
| | TTATGGTTGAATATAATGCTGTACTTGTTTTAGAGAATTTGAATTTTAGATTTAA | |
| | AGAAAAACAAATGAAATTTGAAAGTAATGTTTATCAAAAATTTGAAAAGATGCT | |
| | TATTGATAAATTGAATTTCTTAGTTGATAAAAAGCTTGATAAGAACGCCAATGG | |
| | TGGATTGTTTAATGCGTATCAATTAACAGAAAAATTTACAAACTTTAAAGATATG | |
| | AAAAATCAAAATGGTATAATATTTTATATTCCTGCTTGGATGACAAGCAAAATT | |
| | GACCCAGTTACAGGATTTACAAATTTATTCTATATTAAATATGAGAGTATTGAA | |
| | AAGGCTAAAGAGTTTTTTGGTAAGTTTAAATCAATAAAATTTAATAAGGTAGAC | |
| | AACTATTTTGAATTTGAATTTGATTATAATGATTTTACTGACAGAGCTCAAGGTA | |
| | CAAGGTCTAAATGGACAGTTTGTAGTTTTGGCCCTAGAATTGAAGGTTTTAGA | |
| | AATCCTGAAAAAAATAATAGTTGGGATGGTAGAGAAATAGATATAACAGAGAA | |
| | AATTAAAAAATTACTTGATGATTATAATGTATOTTTAGATAAAGATATTAAAGCT | |
| | CAAATTATGGATATAAATACTAAGGATTTCTTTGAAAAATTTATTAAATATTTTAA | |
| | ACTTGTATTGCAAATGAGAAACAGTAAAACAGGTACAGATATTGATTATATCAT | |
| | TTCTCCGGTTAAAAATAAGCAAAATGAATTTTTTGACAGTAGAAAGCAAAATGA | |
| | AAAAATGCCTATGGATGCAGATGCAAATGGTGCTTATAATATTGCTAGAAAAG | |
| | GCTTAATGTTTATTGATATAATAAAAGAAACTGAAGATAAAGATTTAAAGATGC | |
| | CTAAATTGTTCATTAAAAATAAAGATTGGTTAAATTATGTACAAAAGAGTGATTT | |
| | GTAA | |
| Codon | AATATCTATGAGAACTTCACCAACATGTACCAGGTGAACAAGACAATCCGCAT | 17 |
| optimized | GGGCCTGAAGCCTATCTGTAAAACCGGAGAAAACATCGCCAAGTTCCTGGAG | |
| coding | GAGGACAAGGAAACCAGCGACAAGTACAAGATCGCCAAGGAGGTCATCGACA | |
| sequence (no | AGGAGAACAGAGCCTTTATCGAGGACAGACTGAAGGACTTCAGCATCAGCGG | |
| N-terminal | CCTGGACGAGTACCTGGAACTGCTGAAGCAGAAGAAAAACCTGACAAAGAAC | |
| methionine, no | CAGAACAAGATGAAAAAGGAAATCTCCACCCAGCTGACAAAGATCCAGAACAA | |
| stop codon) | GATGCGGGACGAGATATCGACACAGCTGAAGGGCTTCCCTCAGTTCGATAAC | |
| | AAATACAAGTTCAAATATATCACAGACAAGGAGGACATCGAAATCCTCAAGTA | |
| | CTTCAAGGATAAGAAGTTCATTACATTCTTTGAGGAATTTAATACCAATCGGAA | |
| | AAACGTGTACAGCAAGGAAAACATCAGCACCTCTATCGGCCATAGAATCGTG | |
| | CACGAGAACCTGCCAAAGTTCATCAGCAACTTCAGAATCCTGAATAAGGCCAT | |
| | CGAGGCCTTCGGCATCTCTAAAATCAATGAGGACTTCAAGAACAATGGCATCA | |
| | ACGTGACCGTAGAAGAACTGAACAAGATCGACTACTTCAACAAGGTCCTGACA | |
| | CAGAGCGGCATTGACCTGTACAACAACCTGATTGGCATCCTGAACCAGAACA | |
| | TCAACCTGTACAATCAGCAGCAGAAGGTGAAGAAGAACAAAATCGGAAAGCT | |
| | GGAAATCCTGTACAAGCAAATCTTGTCCAAAACCGACAAGGTGTCTTTCATTG | |
| | AGGAGTTCACCGAGGACAACCAGCTGCTGGAGTGCATCGACGAGTACTTTAA | |
| | AGAGAAATACAGCCTGATCACCGTGGACCTGAACAACCTGCTTGAAAATATCG | |
| | ACACCTACAATCTCAACGGCATCTTCATCAAATCTGATAAAAGCCTGGGCAAC | |
| | ATCAGCAACTACCTGTACAAGGATTGGTGGTACATCAGCAACCTGATCAACGA | |
| | AGAATACGACTACAAGCACAAGAACAAGGTCAGAGATGATAAGTACTACGAGA | |
| | CAAGAAAGAAGGCCATCGACAAGATCAAGTACTTCTCTATCGGACACATCGAT | |
| | GAGCTGCTGAAGGACAAGAACGTTCCAATGGTGGAAAACTACTTCAAGGAGA | |
| | AGATCAACCTGGTCGTGAAGGAGTTCAATGCTTATCTGAACAAGTTCAATGAA | |
| | TATAAATTCATCAACGAGCTGAAAACAGACGAGATCGCCGTGGAAATCATCAA | |
| | GAACCTGTGCGACAGCATCAAGAACGTGCAGGGCATCGTGAAGCCCCTGATC | |
| | ATCACCGGCAACGACAAGGATGATGATTTTTACGTGGAGATCAACTACATCTG | |
| | GGATGAGCTTAACAAGTTCGACAAAATCTACAACATGGTCAGGAATTACCTAA | |
| | CCAAGAAGGACTACATCGAGGAAAAGATCAGAATGATGTTTTCCAAGAGCAG | |
| | CTTTATGGACGGCTGGGGCAAGGACTACGGCACCAAGAAGGCCCACATCGT | |
| | GTACCACGACAAGAACTACTACCTGGTGATCGTGGACAAGAAGCTGAAACTG | |
| | GAAGATATCGACAAACTATACAAGCCAGGCGGCGACACAGTTCACTACGTGT | |
| | ACAACTACCAGTCTACCGAGAACGGAAACATCCCTCGGAAGTTCATCTACTCT | |
| | AAGGGCAAGCGGTTCGCCCCTAGCGTGGAAAAATATAACCTGCCTATTGAAG | |
| | ATGTGATTGAGGTGTACAACAACGAGTACCACACCACCGACTATGAGAAAAAG | |
| | AACCCTGAGATATACAAAAAGTCCCTGACCAGCCTGATCGACTATTTCAAGAT | |
| | CGGCGTGAACAGAGATATGGACTTCGAGAAGTTTGATTTTCGGCTAAAGGACT | |
| | CCAACGAATACAAGAACATCAAGGAGTTCTACGATAACCTGGAGACATGCTGC | |
| | TACAAGCTGCAGGAGGAAAAGGTGAACTTCAACGTGCTGGAGGAACTGAGCT | |
| | ACAGCGGAAAGATCTACCTGTTCAAGATCTACAACAAAGATTTCAGCGAGAAT | |
| | AGCAAAGGCATCCCTAACCTGCATACCCTGTACTTCAAAATGCTGTTCGACAA | |
| | AGAGAACCTGGAGAACCCCATCTACAAGCTGTCTGGAAAAGCTAAGATGTTTT | |
| | TCAGAAAGGGCAGCCTGAACCTGGACAAAAAAACCGTTGACTATGACAAAAAA | |
| | CCTATCGATAAGAAGGAAAACGACAAAAAAATCAAGAATAGGCGGTACAAGGT | |
| | GGACAGCTTCACCCTGCACATGAGCATCATCACCAACTTCCAGAGCTACGAG | |
| | AACAAGAACGTTAATGAGACTGTGAACCGGGCCCTGAAGTACTGCGACGACG | |
| | TGTACGCCATCGGCATCGACCGCGGAATCCGGAACCTGCTGTACGCTTGTGT | |
| | GGTGAACAGCAAGGGCGAGATCGTGAAGCAAGTGCCCCTCAACATCATTAAC | |
| | AATAAGGATTACCACAACCTGCTGGCCGAGAGAGAAGAAAAGAAGAAAAACA | |
| | GCAGAAAGAATTGGAAGATCATAGACAACATCAGAAACCTGAAGGAAGGCTA | |
| | CCTGAGCCAGGCCATCCACATCATCACCGACCTGATGGTGGAATACAACGCC | |
| | GTGCTGGTGCTGGAGAACCTGAATTTCAGATTCAAGGAGAAGCAGATGAAGT | |
| | TTGAAAGCAATGTGTACCAAAAATTCGAAAAAATGCTGATCGACAAGCTGAAT | |
| | TTCCTGGTCGATAAAAAACTGGACAAGAATGCCAATGGCGGACTGTTTAACGC | |
| | CTATCAGCTGACAGAGAAGTTCACCAACTTTAAGGATATGAAGAATCAGAACG | |
| | GCATCATCTTCTACATCCCCGCCTGGATGACAAGCAAGATCGATCCCGTGAC | |
| | CGGCTTCACAAACCTGTTTTATATCAAATACGAGAGCATCGAGAAGGCAAAGG | |
| | AGTTCTTCGGCAAGTTTAAGTCTATCAAGTTCAATAAGGTGGACAATTATTTCG | |
| | AGTTCGAGTTCGACTACAACGACTTTACCGACAGAGCTCAAGGCACCAGAAG | |
| | CAAGTGGACCGTGTGTAGCTTCGGTCCTCGGATCGAGGGCTTCAGAAACCCC | |
| | GAGAAAAACAATTCCTGGGACGGCAGAGAAATCGACATCACAGAGAAGATCA | |
| | AGAAGCTGCTGGATGACTACAATGTGAGCCTGGACAAAGACATCAAAGCCCA | |
| | GATCATGGACATCAACACCAAGGATTTCTTCGAGAAGTTCATCAAGTACTTCA | |
| | AGCTGGTGCTGCAGATGAGAAACAGCAAGACCGGCACCGACATCGATTACAT | |
| | TATCTCCCCTGTGAAGAACAAGCAGAACGAGTTTTTCGACTCCAGAAAGCAGA | |
| | ACGAGAAGATGCCTATGGACGCTGATGCCAACGGCGCCTACAACATCGCTAG | |
| | AAAGGGGCTGATGTTCATCGATATCATCAAGGAAACAGAGGACAAGGACCTG | |
| | AAAATGCCTAAGCTGTTCATAAAGAACAAGGATTGGCTGAACTATGTGCAGAA | |
| | ATCAGATCTG | |
| Expression | ATGggcAATATCTATGAGAACTTCACCAACATGTACCAGGTGAACAAGACAATC | 18 |
| construct (with | CGCATGGGCCTGAAGCCTATCTGTAAAACCGGAGAAAACATCGCCAAGTTCC | |
| N-terminal | TGGAGGAGGACAAGGAAACCAGCGACAAGTACAAGATCGCCAAGGAGGTCA | |
| methionine | TCGACAAGGAGAACAGAGCCTTTATCGAGGACAGACTGAAGGACTTCAGCAT | |
| and stop | CAGCGGCCTGGACGAGTACCTGGAACTGCTGAAGCAGAAGAAAAACCTGACA | |
| codon, | AAGAACCAGAACAAGATGAAAAAGGAAATCTCCACCCAGCTGACAAAGATCCA | |
| includes V5- | GAACAAGATGCGGGACGAGATATCGACACAGCTGAAGGGCTTCCCTCAGTTC | |
| tag and C- | GATAACAAATACAAGTTCAAATATATCACAGACAAGGAGGACATCGAAATCCT | |
| terminal NLS) | CAAGTACTTCAAGGATAAGAAGTTCATTACATTCTTTGAGGAATTTAATACCAA | |
| | TCGGAAAAACGTGTACAGCAAGGAAAACATCAGCACCTCTATCGGCCATAGA | |
| | ATCGTGCACGAGAACCTGCCAAAGTTCATCAGCAACTTCAGAATCCTGAATAA | |
| | GGCCATCGAGGCCTTCGGCATCTCTAAAATCAATGAGGACTTCAAGAACAATG | |
| | GCATCAACGTGACCGTAGAAGAACTGAACAAGATCGACTACTTCAACAAGGTC | |
| | CTGACACAGAGCGGCATTGACCTGTACAACAACCTGATTGGCATCCTGAACC | |
| | AGAACATCAACCTGTACAATCAGCAGCAGAAGGTGAAGAAGAACAAAATCGG | |
| | AAAGCTGGAAATCCTGTACAAGCAAATCTTGTCCAAAACCGACAAGGTGTCTT | |
| | TCATTGAGGAGTTCACCGAGGACAACCAGCTGCTGGAGTGCATCGACGAGTA | |
| | CTTTAAAGAGAAATACAGCCTGATCACCGTGGACCTGAACAACCTGCTTGAAA | |
| | ATATCGACACCTACAATCTCAACGGCATCTTCATCAAATCTGATAAAAGCCTG | |
| | GGCAACATCAGCAACTACCTGTACAAGGATTGGTGGTACATCAGCAACCTGAT | |
| | CAACGAAGAATACGACTACAAGCACAAGAACAAGGTCAGAGATGATAAGTACT | |
| | ACGAGACAAGAAAGAAGGCCATCGACAAGATCAAGTACTTCTCTATCGGACA | |
| | CATCGATGAGCTGCTGAAGGACAAGAACGTTCCAATGGTGGAAAACTACTTCA | |
| | AGGAGAAGATCAACCTGGTCGTGAAGGAGTTCAATGCTTATCTGAACAAGTTC | |
| | AATGAATATAAATTCATCAACGAGCTGAAAACAGACGAGATCGCCGTGGAAAT | |
| | CATCAAGAACCTGTGCGACAGCATCAAGAACGTGCAGGGCATCGTGAAGCCC | |
| | CTGATCATCACCGGCAACGACAAGGATGATGATTTTTACGTGGAGATCAACTA | |
| | CATCTGGGATGAGCTTAACAAGTTCGACAAAATCTACAACATGGTCAGGAATT | |
| | ACCTAACCAAGAAGGACTACATCGAGGAAAAGATCAGAATGATGTTTTCCAAG | |
| | AGCAGCTTTATGGACGGCTGGGGCAAGGACTACGGCACCAAGAAGGCCCAC | |
| | ATCGTGTACCACGACAAGAACTACTACCTGGTGATCGTGGACAAGAAGCTGA | |
| | AACTGGAAGATATCGACAAACTATACAAGCCAGGCGGCGACACAGTTCACTA | |
| | CGTGTACAACTACCAGTCTACCGAGAACGGAAACATCCCTCGGAAGTTCATCT | |
| | ACTCTAAGGGCAAGCGGTTCGCCCCTAGCGTGGAAAAATATAACCTGCCTATT | |
| | GAAGATGTGATTGAGGTGTACAACAACGAGTACCACACCACCGACTATGAGA | |
| | AAAAGAACCCTGAGATATACAAAAAGTCCCTGACCAGCCTGATCGACTATTTC | |
| | AAGATCGGCGTGAACAGAGATATGGACTTCGAGAAGTTTGATTTTCGGCTAAA | |
| | GGACTCCAACGAATACAAGAACATCAAGGAGTTCTACGATAACCTGGAGACAT | |
| | GCTGCTACAAGCTGCAGGAGGAAAAGGTGAACTTCAACGTGCTGGAGGAACT | |
| | GAGCTACAGCGGAAAGATCTACCTGTTCAAGATCTACAACAAAGATTTCAGCG | |
| | AGAATAGCAAAGGCATCCCTAACCTGCATACCCTGTACTTCAAAATGCTGTTC | |
| | GACAAAGAGAACCTGGAGAACCCCATCTACAAGCTGTCTGGAAAAGCTAAGA | |
| | TGTTTTTCAGAAAGGGCAGCCTGAACCTGGACAAAAAAACCGTTGACTATGAC | |
| | AAAAAACCTATCGATAAGAAGGAAAACGACAAAAAAATCAAGAATAGGCGGTA | |
| | CAAGGTGGACAGCTTCACCCTGCACATGAGCATCATCACCAACTTCCAGAGC | |
| | TACGAGAACAAGAACGTTAATGAGACTGTGAACCGGGCCCTGAAGTACTGCG | |
| | ACGACGTGTACGCCATCGGCATCGACCGCGGAATCCGGAACCTGCTGTACG | |
| | CTTGTGTGGTGAACAGCAAGGGCGAGATCGTGAAGCAAGTGCCCCTCAACAT | |
| | CATTAACAATAAGGATTACCACAACCTGCTGGCCGAGAGAGAAGAAAAGAAG | |
| | AAAAACAGCAGAAAGAATTGGAAGATCATAGACAACATCAGAAACCTGAAGGA | |
| | AGGCTACCTGAGCCAGGCCATCCACATCATCACCGACCTGATGGTGGAATAC | |
| | AACGCCGTGCTGGTGCTGGAGAACCTGAATTTCAGATTCAAGGAGAAGCAGA | |
| | TGAAGTTTGAAAGCAATGTGTACCAAAAATTCGAAAAAATGCTGATCGACAAG | |
| | CTGAATTTCCTGGTCGATAAAAAACTGGACAAGAATGCCAATGGCGGACTGTT | |
| | TAACGCCTATCAGCTGACAGAGAAGTTCACCAACTTTAAGGATATGAAGAATC | |
| | AGAACGGCATCATCTTCTACATCCCCGCCTGGATGACAAGCAAGATCGATCC | |
| | CGTGACCGGCTTCACAAACCTGTTTTATATCAAATACGAGAGCATCGAGAAGG | |
| | CAAAGGAGTTCTTCGGCAAGTTTAAGTCTATCAAGTTCAATAAGGTGGACAAT | |
| | TATTTCGAGTTCGAGTTCGACTACAACGACTTTACCGACAGAGCTCAAGGCAC | |
| | CAGAAGCAAGTGGACCGTGTGTAGCTTCGGTCCTCGGATCGAGGGCTTCAGA | |
| | AACCCCGAGAAAAACAATTCCTGGGACGGCAGAGAAATCGACATCACAGAGA | |
| | AGATCAAGAAGCTGCTGGATGACTACAATGTGAGCCTGGACAAAGACATCAA | |
| | AGCCCAGATCATGGACATCAACACCAAGGATTTCTTCGAGAAGTTCATCAAGT | |
| | ACTTCAAGCTGGTGCTGCAGATGAGAAACAGCAAGACCGGCACCGACATCGA | |
| | TTACATTATCTCCCCTGTGAAGAACAAGCAGAACGAGTTTTTCGACTCCAGAA | |
| | AGCAGAACGAGAAGATGCCTATGGACGCTGATGCCAACGGCGCCTACAACAT | |
| | CGCTAGAAAGGGGCTGATGTTCATCGATATCATCAAGGAAACAGAGGACAAG | |
| | GACCTGAAAATGCCTAAGCTGTTCATAAAGAACAAGGATTGGCTGAACTATGT | |
| | GCAGAAATCAGATCTGtctagaAAGCGGACAGCAGACGGCTCCGAATTTGAAAG | |
| | CCCTAAGAAAAAGAGAAAGGTGggatccGGCAAACCTATCCCCAATCCCCTGCT | |
| | GGGCCTGGACAGCACCTGA | |

In some embodiments a ZIKV Type V Cas protein comprises an amino acid sequence of SEQ ID NO: 13, SEQ ID NO: 14, or SEQ ID NO: 15. In some embodiments, a ZIKV Type V Cas protein has nickase activity, for example resulting from one or more amino acid substitutions relative to the sequence of SEQ ID NO: 13, SEQ ID NO: 14, or SEQ ID NO: 15. In some embodiments, the one or more amino acid substitutions providing nickase activity comprise a D814 substitution, wherein the position of the D814 substitution is defined with respect to the amino acid numbering of SEQ ID NO: 14 (corresponding to amino acid 908 of SEQ ID NO: 121). In some embodiments, the one or more amino acid substitutions providing nickase activity comprise an E899 substitution, wherein the position of the E899 substitution is defined with respect to the amino acid numbering of SEQ ID NO: 14 (corresponding to amino acid 993 of SEQ ID NO: 121). In some embodiments, the one or more amino acid substitutions providing nickase activity comprise a R1111 substitution, wherein the position of the R1111 substitution is defined with respect to the amino acid numbering of SEQ ID NO: 14 (corresponding to amino acid 1226 of SEQ ID NO: 121). In some embodiments, the one or more amino acid substitutions providing nickase activity comprise a D1148 substitution, wherein the position of the D1148 substitution is defined with respect to the amino acid numbering of SEQ ID NO: 14 (corresponding to amino acid 1263 of SEQ ID NO:121). In some embodiments, a ZIKV Type V Cas protein is catalytically inactive, for example due to a R1111 substitution in combination with a D814 substitution, a E899 substitution, and/or D1148 substitution.

### 6.2.4. ZZFT Type V Cas Proteins

In one aspect, the disclosure provides ZZFT Type V Cas proteins. ZZFT Type V Cas proteins can be further classified as Type V-A Cas proteins. The ZZFT Type V Cas proteins typically comprise an amino acid sequence that is at least 50%, at least 55%, at least 60%, at least 65%, at least 70%, at least 75%, at least 85%, at least 90%, or at least 95% identical to SEQ ID NO: 19. In some embodiments, the ZZFT Type V Cas proteins comprise an amino acid sequence that is at least 95%, at least 96%, at least 97%, at least 98%, or at least 99% identical to SEQ ID NO: 19. In some embodiments, a ZZFT Type V Cas protein comprises an amino acid sequence that is identical to SEQ ID NO: 19.

Exemplary ZZFT Type V Cas protein sequences and nucleotide sequences encoding exemplary ZZFT Type V Cas proteins are set forth in Table 1D.

TABLE 1D
ZZFT Type V Cas Sequences

| | | SEQ ID |
|---|---|---|
| Name | Sequence | NO. |
| Wildtype | EISNRFTNKYQVSKTLRFRLEPTGGTDDLLCQAQIIEGDERRNKEAITMKQILDNC | 19 |
| amino acid | HKQIIERVLSDFNFKEHSLEEFFKVYTRNDDDREKDIENLQAKMRKEIAAAFTKQD | |
| sequence | VTKLFSSKFKDFVERGLIKYASNEKERNIVSRFKGFATYFTGFNTNRLNMYSEEAK | |
| (without N- | STAISFRLINQNLIKFIDNILVYKKVSQTLPSDVLSNIYIDFKAIINTSSLEEFFSINNYN | |
| terminal | NILTQKQIEIFNAVIGGKKDKDEKIITKGFNQYINEYNQTNKNIRLPKMMRLFNQILS | |
| methionine) | DREGVSARPEPFNNANETISSVRDCFTNEISKQITILSETTSKIESFDIDRIYIKGGE | |
| | DLRALSNSIYGYFNYIHDRIADKWKHNNPQGKKSPESYQKNLNAYLKGIKSVSLHS | |
| | IANICGDNKVIEYFRNLGAENTVDFQRENVVSLIDNKYNCASNLLSDAQITDEELRT | |
| | NSRSIKDLLDAVKSAQRFFRLLCGSGNEPDKDHSFYDEYTPAFEALENSINPLYNK | |
| | VRSFVTKKDFSTDKFKLNFDSSSFLSGWAKKSEYEKSSAFIFIRDNQYYLGINKCL | |
| | SKEDIAYLEDSTSSSDTKRVVYMFQKVDATNIPRIFIRSKGSNLAPAVNEFQLPIETI | |
| | LDIYDNKFFTTSYQKKDRTKWKESLTKLIDYYKLGFSQHKSYADFDLKWKASSEY | |
| | NDINDFLADVQRFCYRIEFININWDKLIEFTEDGKFYLFRIANKDLSGNSTGLPNLH | |
| | TIYWKMLFDESNLKDIVYKLSGNAEVFMRYNSLKNPIVHKAGVEIKNKCPFTEKKT | |
| | SIFDYDIIKDRRYTKDQLELHVPILMNFKSPSAAKGKAFNKECLEYIRNNGIKHIIGID | |
| | RGERNLLYMVITDLDGNIVEQKSLNQIASNPKLPLFRQDYNKLLKTKADANAQARR | |
| | DWETIDTVKEIKFGFLSQIVHEIAMAIIKYDAIVVLENLNRGFMQKRGLENNVYQKF | |
| | EQMLLDKLSYYVDKTKHPEEAGGALHAYQLSDTYANFNSLSKNAMVRQSGFVFY | |
| | IPAWLTSKIDPVTGFASFLKFHRDDSMATIKSTISKFDCFKYDKECDMFHIRIDYNK | |
| | FSTSCSGGQRKWDLFTFGDRILAERNTMQNSRYVYQTVNLTSEFKNLFATKDIDI | |
| | SGNLKDSICKIEDVGFFRKLSQLLSLTLQLRNSNAETGEDFLISPVADKDGNFFDS | |
| | RNCPDSLPKDADANGAYNIARKGLMLVEQLKRCKDVSKFKPAIKNEDWLDYVQR | |
| Wildtype | MEISNRFTNKYQVSKTLRFRLEPTGGTDDLLCQAQIIEGDERRNKEAITMKQILDN | 20 |
| amino acid | CHKQIIERVLSDFNFKEHSLEEFFKVYTRNDDDREKDIENLQAKMRKEIAAAFTKQ | |
| sequence (with | DVTKLFSSKFKDFVERGLIKYASNEKERNIVSRFKGFATYFTGFNTNRLNMYSEEA | |
| N-terminal | KSTAISFRLINQNLIKFIDNILVYKKVSQTLPSDVLSNIYIDFKAIINTSSLEEFFSINNY | |
| methionine) | NNILTQKQIEIFNAVIGGKKDKDEKIITKGFNQYINEYNQTNKNIRLPKMMRLFNQIL | |
| | SDREGVSARPEPFNNANETISSVRDCFTNEISKQITILSETTSKIESFDIDRIYIKGG | |
| | EDLRALSNSIYGYFNYIHDRIADKWKHNNPQGKKSPESYQKNLNAYLKGIKSVSLH | |
| | SIANICGDNKVIEYFRNLGAENTVDFQRENVVSLIDNKYNCASNLLSDAQITDEELR | |
| | TNSRSIKDLLDAVKSAQRFFRLLCGSGNEPDKDHSFYDEYTPAFEALENSINPLYN | |
| | KVRSFVTKKDFSTDKFKLNFDSSSFLSGWAKKSEYEKSSAFIFIRDNQYYLGINKC | |
| | LSKEDIAYLEDSTSSSDTKRVVYMFQKVDATNIPRIFIRSKGSNLAPAVNEFQLPIE | |
| | TILDIYDNKFFTTSYQKKDRTKWKESLTKLIDYYKLGFSQHKSYADFDLKWKASSE | |
| | YNDINDFLADVQRFCYRIEFININWDKLIEFTEDGKFYLFRIANKDLSGNSTGLPNL | |
| | HTIYWKMLFDESNLKDIVYKLSGNAEVFMRYNSLKNPIVHKAGVEIKNKCPFTEKK | |
| | TSIFDYDIIKDRRYTKDQLELHVPILMNFKSPSAAKGKAFNKECLEYIRNNGIKHIIGI | |
| | DRGERNLLYMVITDLDGNIVEQKSLNQIASNPKLPLFRQDYNKLLKTKADANAQAR | |
| | RDWETIDTVKEIKFGFLSQIVHEIAMAIIKYDAIVVLENLNRGFMQKRGLENNVYQK | |
| | FEQMLLDKLSYYVDKTKHPEEAGGALHAYQLSDTYANFNSLSKNAMVRQSGFVF | |
| | YIPAWLTSKIDPVTGFASFLKFHRDDSMATIKSTISKFDCFKYDKECDMFHIRIDYN | |
| | KFSTSCSGGQRKWDLFTFGDRILAERNTMQNSRYVYQTVNLTSEFKNLFATKDID | |
| | ISGNLKDSICKIEDVGFFRKLSQLLSLTLQLRNSNAETGEDFLISPVADKDGNFFDS | |
| | RNCPDSLPKDADANGAYNIARKGLMLVEQLKRCKDVSKFKPAIKNEDWLDYVQR | |
| Expression | MGEISNRFTNKYQVSKTLRFRLEPTGGTDDLLCQAQIIEGDERRNKEAITMKQILD | 21 |
| construct (with | NCHKQIIERVLSDFNFKEHSLEEFFKVYTRNDDDREKDIENLQAKMRKEIAAAFTK | |
| N-terminal | QDVTKLFSSKFKDFVERGLIKYASNEKERNIVSRFKGFATYFTGFNTNRLNMYSE | |
| methionine, | EAKSTAISFRLINQNLIKFIDNILVYKKVSQTLPSDVLSNIYIDFKAIINTSSLEEFFSIN | |
| V5-tag and C- | NYNNILTQKQIEIFNAVIGGKKDKDEKIITKGFNQYINEYNQTNKNIRLPKMMRLFN | |
| terminal NLS) | QILSDREGVSARPEPFNNANETISSVRDCFTNEISKQITILSETTSKIESFDIDRIYIK | |
| aa sequence | GGEDLRALSNSIYGYFNYIHDRIADKWKHNNPQGKKSPESYQKNLNAYLKGIKSV | |
| | SLHSIANICGDNKVIEYFRNLGAENTVDFQRENVVSLIDNKYNCASNLLSDAQITDE | |
| | ELRTNSRSIKDLLDAVKSAQRFFRLLCGSGNEPDKDHSFYDEYTPAFEALENSINP | |
| | LYNKVRSFVTKKDFSTDKFKLNFDSSSFLSGWAKKSEYEKSSAFIFIRDNQYYLGI | |
| | NKCLSKEDIAYLEDSTSSSDTKRVVYMFQKVDATNIPRIFIRSKGSNLAPAVNEFQ | |
| | LPIETILDIYDNKFFTTSYQKKDRTKWKESLTKLIDYYKLGFSQHKSYADFDLKWKA | |
| | SSEYNDINDFLADVQRFCYRIEFININWDKLIEFTEDGKFYLFRIANKDLSGNSTGL | |
| | PNLHTIYWKMLFDESNLKDIVYKLSGNAEVFMRYNSLKNPIVHKAGVEIKNKCPFT | |
| | EKKTSIFDYDIIKDRRYTKDQLELHVPILMNFKSPSAAKGKAFNKECLEYIRNNGIK | |
| | HIIGIDRGERNLLYMVITDLDGNIVEQKSLNQIASNPKLPLFRQDYNKLLKTKADAN | |
| | AQARRDWETIDTVKEIKFGFLSQIVHEIAMAIIKYDAIVVLENLNRGFMQKRGLENN | |
| | VYQKFEQMLLDKLSYYVDKTKHPEEAGGALHAYQLSDTYANFNSLSKNAMVRQS | |
| | GFVFYIPAWLTSKIDPVTGFASFLKFHRDDSMATIKSTISKFDCFKYDKECDMFHIR | |
| | IDYNKFSTSCSGGQRKWDLFTFGDRILAERNTMQNSRYVYQTVNLTSEFKNLFAT | |
| | KDIDISGNLKDSICKIEDVGFFRKLSQLLSLTLQLRNSNAETGEDFLISPVADKDGN | |
| | FFDSRNCPDSLPKDADANGAYNIARKGLMLVEQLKRCKDVSKFKPAIKNEDWLD | |
| | YVQRSRKRTADGSEFESPKKKRKVGSGKPIPNPLLGLDST | |
| Wildtype | ATGGAAATTTCGAACCGATTCACAAACAAGTATCAAGTAAGCAAGACCCTCCG | 22 |
| coding | CTTTCGCCTTGAGCCAACCGGAGGTACTGATGATTTACTTTGCCAAGCACAAA | |
| sequence (with | TCATCGAGGGAGACGAGCGCCGCAATAAAGAGGCTATAACAATGAAACAGAT | |
| N-terminal | TTTGGACAATTGTCACAAACAGATAATTGAGCGCGTATTGTCCGACTTTAATTT | |
| methionine | TAAAGAGCATTCTCTTGAAGAGTTTTTCAAAGTGTATACCAGAAACGATGATGA | |
| and stop | CCGCGAAAAGGACATTGAAAATCTCCAAGCAAAAATGCGCAAAGAAATAGCC | |
| codon) | GCCGCCTTCACCAAACAGGATGTTACGAAACTTTTCTCAAGCAAATTCAAGGA | |
| | TTTTGTTGAAAGAGGCTTGATTAAATATGCATCAAACGAGAAGGAACGCAACA | |
| | TCGTTTCCCGCTTCAAAGGTTTTGCCACTTACTTTACAGGGTTCAATACCAATA | |
| | GACTGAATATGTACTCAGAAGAAGCAAAATCCACAGCTATATCATTCAGATTAA | |
| | TTAATCAAAACTTGATAAAGTTCATAGACAACATCCTTGTATATAAAAAAGTGT | |
| | CTCAAACGTTGCCTTCAGATGTGCTATCAAACATTTATATAGACTTTAAGGCAA | |
| | TCATCAACACATCAAGTCTTGAAGAATTCTTCTCCATAAACAACTACAATAACA | |
| | TACTCACCCAGAAACAGATTGAGATTTTCAATGCAGTTATCGGAGGTAAAAAA | |
| | GACAAGGATGAAAAAATAATAACCAAAGGATTCAACCAATATATAAACGAATAC | |
| | AACCAGACCAATAAAAACATCCGTCTGCCTAAGATGATGCGGTTATTCAATCA | |
| | AATCCTAAGCGACAGAGAAGGTGTTTCTGCAAGACCAGAGCCATTCAATAACG | |
| | CGAACGAGACAATCAGTTCCGTCCGTGATTGTTTTACAAACGAAATATCAAAA | |
| | CAAATAACGATATTGTCTGAAACAACATCCAAAATTGAATCATTCGACATTGAT | |
| | AGAATTTACATTAAGGGCGGAGAAGATCTGAGAGCATTATCCAACAGTATATA | |
| | TGGATATTTCAATTATATCCATGACCGTATCGCAGACAAATGGAAACACAACAA | |
| | TCCTCAGGGCAAAAAGAGCCCCGAAAGCTACCAAAAAAACCTCAACGCATAT | |
| | CTGAAAGGCATAAAAAGCGTCTCTTTACACAGTATTGCAAACATCTGTGGTGA | |
| | CAACAAAGTTATTGAGTATTTCAGGAATCTTGGTGCAGAAAACACTGTTGATTT | |
| | CCAAAGAGAGAACGTTGTATCATTAATCGACAACAAATACAACTGCGCTTCAA | |
| | ATCTTTTATCCGACGCCCAAATTACGGATGAAGAACTTCGCACAAACAGTCGC | |
| | TCAATTAAAGACTTGCTTGACGCCGTCAAGAGTGCCCAACGATTTTTCCGTCT | |
| | ACTGTGCGGTTCTGGCAACGAACCAGACAAAGACCACTCTTTTTATGACGAGT | |
| | ATACACCAGCATTTGAAGCACTTGAGAATTCAATAAATCCCCTATATAACAAAG | |
| | TCAGGAGTTTTGTAACCAAAAAAGATTTCTCCACCGATAAATTCAAATTGAATT | |
| | TCGACAGCAGCAGCTTTCTATCCGGTTGGGCAAAGAAATCAGAATATGAGAA | |
| | GAGTTCTGCATTTATATTTATTCGCGACAATCAATATTACTTAGGAATAAACAA | |
| | ATGCCTTAGCAAAGAAGACATTGCCTACCTTGAGGACTCAACAAGCTCATCAG | |
| | ATACAAAAAGAGTGGTATATATGTTCCAAAAAGTGGACGCCACGAATATTCCC | |
| | AGAATATTCATCCGTTCCAAAGGTTCCAATTTAGCTCCTGCTGTCAACGAATTC | |
| | CAACTGCCGATAGAAACCATTCTTGACATTTATGACAATAAGTTCTTCACTACC | |
| | AGTTATCAGAAAAAAGACCGGACTAAATGGAAAGAATCATTGACCAAACTCAT | |
| | TGACTATTACAAGCTTGGATTCAGCCAGCACAAGTCATACGCAGATTTCGACT | |
| | TAAAATGGAAAGCATCCAGTGAATATAACGACATAAATGACTTTCTTGCAGAC | |
| | GTACAGAGATTCTGCTACAGAATCGAATTTATAAATATCAATTGGGACAAGCT | |
| | GATAGAATTCACAGAAGATGGCAAATTTTACCTATTCCGCATTGCAAATAAAGA | |
| | TTTATCAGGCAATAGCACAGGTCTGCCCAATTTGCACACGATTTATTGGAAAA | |
| | TGCTTTTTGACGAAAGCAACCTCAAAGATATTGTCTATAAATTGTCGGGCAATG | |
| | CGGAAGTCTTTATGCGCTATAATTCATTAAAAAATCCAATTGTGCATAAAGCGG | |
| | GAGTGGAGATTAAAAACAAATGCCCTTTTACTGAAAAAAAGACAAGCATATTTG | |
| | ACTACGACATTATAAAAGACCGTCGCTATACAAAAGATCAGCTTGAACTGCAT | |
| | GTTCCAATCCTAATGAACTTCAAAAGCCCATCGGCAGCAAAAGGCAAAGCTTT | |
| | CAACAAAGAATGCTTGGAATACATAAGAAATAATGGTATAAAGCATATTATAGG | |
| | AATAGACCGAGGTGAACGGAATCTACTTTATATGGTTATAACAGACCTTGACG | |
| | GCAACATCGTTGAGCAAAAGTCTTTGAACCAAATTGCGAGCAATCCGAAATTG | |
| | CCTCTTTTCAGACAAGACTACAACAAGCTGCTGAAGACAAAGGCTGATGCAAA | |
| | CGCACAAGCACGTCGTGATTGGGAAACAATAGACACCGTAAAGGAGATAAAA | |
| | TTCGGCTTCTTGAGTCAGATTGTACATGAGATAGCAATGGCTATCATAAAATAC | |
| | GATGCAATTGTTGTTTTGGAGAATCTGAACAGAGGGTTTATGCAGAAACGAGG | |
| | TCTTGAAAACAACGTCTATCAGAAATTCGAACAAATGCTGCTTGACAAGTTGA | |
| | GCTACTATGTCGACAAAACGAAACATCCGGAAGAGGCCGGAGGAGCTTTGCA | |
| | CGCATATCAGCTCTCTGACACTTACGCGAACTTCAATTCTCTGTCGAAGAATG | |
| | CGATGGTGCGACAGTCGGGTTTTGTTTTCTATATTCCTGCATGGCTTACAAGC | |
| | AAAATAGACCCCGTCACAGGATTCGCCTCCTTTTTGAAATTTCACAGAGATGA | |
| | CAGTATGGCAACAATCAAATCTACAATTTCAAAGTTTGATTGTTTCAAATACGA | |
| | CAAGGAATGCGACATGTTCCACATCCGCATTGACTATAACAAGTTTAGCACAA | |
| | GCTGCAGCGGAGGTCAACGCAAATGGGACTTGTTCACTTTTGGCGATCGAAT | |
| | CTTGGCAGAACGCAATACAATGCAAAACAGCAGATATGTTTACCAAACAGTCA | |
| | ATTTAACTTCTGAATTCAAAAACTTATTTGCCACAAAGGATATCGACATTTCAG | |
| | GCAACCTGAAGGACTCTATATGCAAAATTGAGGATGTTGGCTTTTTCAGAAAA | |
| | CTAAGCCAACTCTTGTCACTCACGCTTCAATTACGCAACAGCAATGCTGAAAC | |
| | AGGAGAAGACTTCTTGATTTCCCCAGTAGCTGACAAAGATGGCAATTTCTTCG | |
| | ATTCAAGAAACTGTCCCGACTCTCTCCCAAAAGACGCAGATGCCAATGGCGC | |
| | ATACAACATTGCTAGGAAGGGATTAATGCTTGTCGAGCAATTGAAGAGATGCA | |
| | AAGATGTATCAAAATTCAAGCCCGCGATAAAAAACGAGGACTGGTTAGACTAT | |
| | GTTCAACGCTGA | |
| Codon | GAAATCAGTAATCGGTTTACAAACAAGTACCAGGTGTCTAAGACCCTGCGGTT | 23 |
| optimized | CAGACTGGAGCCTACAGGCGGGACCGATGACCTGCTGTGCCAGGCCCAGAT | |
| coding | CATCGAGGGCGATGAGCGGCGCAACAAAGAAGCCATCACCATGAAACAGATC | |
| sequence (no | CTCGACAACTGTCACAAGCAGATCATCGAAAGAGTGCTGTCCGACTTCAACTT | |
| N-terminal | CAAAGAGCACTCCCTGGAAGAGTTCTTTAAGGTGTACACACGGAACGACGAT | |
| methionine, no | GACAGAGAGAAGGATATCGAGAACCTGCAGGCAAAGATGCGCAAGGAAATCG | |
| stop codon) | CCGCCGCCTTTACTAAGCAAGACGTGACAAAACTGTTTTCTTCCAAGTTTAAA | |
| | GACTTTGTCGAAAGGGGTCTGATCAAGTACGCCAGCAACGAGAAGGAGCGGA | |
| | ATATCGTGTCCCGGTTCAAGGGCTTTGCCACATACTTCACCGGCTTCAACACA | |
| | AACCGCCTGAACATGTACAGCGAGGAAGCCAAATCTACGGCCATTAGCTTCC | |
| | GGCTGATCAACCAGAACCTCATCAAATTCATCGACAATATCCTGGTGTACAAG | |
| | AAGGTGTCTCAGACCCTCCCTTCTGATGTCCTGAGCAACATCTACATCGACTT | |
| | CAAGGCCATCATCAATACCAGCAGCCTGGAGGAGTTCTTCTCCATCAACAACT | |
| | ACAACAACATCCTGACCCAGAAGCAGATCGAGATCTTCAACGCTGTGATCGG | |
| | CGGAAAGAAGGATAAGGATGAGAAAATTATCACAAAGGGCTTCAACCAGTACA | |
| | TCAATGAATATAATCAGACCAACAAGAATATCAGACTGCCAAAGATGATGAGA | |
| | CTGTTCAATCAGATACTGAGCGACCGGGAAGGCGTGTCAGCTAGACCTGAGC | |
| | CCTTCAACAACGCCAACGAGACAATCAGCTCCGTGAGAGACTGTTTTACAAAC | |
| | GAAATCAGCAAGCAGATCACCATCCTGTCTGAAACCACCAGTAAGATCGAGA | |
| | GCTTCGACATCGATAGAATCTACATCAAGGGCGGAGAGGACCTGCGGGCCCT | |
| | GAGCAACAGCATCTACGGCTACTTCAACTACATCCACGATAGAATCGCTGATA | |
| | AGTGGAAGCACAACAATCCTCAGGGCAAGAAGAGCCCCGAGAGCTACCAAAA | |
| | GAATCTGAACGCCTACCTGAAGGGCATAAAGAGCGTGAGCCTGCATTCTATC | |
| | GCCAACATCTGTGGCGACAACAAGGTGATCGAATATTTTAGAAATCTCGGCGC | |
| | CGAGAACACAGTGGATTTTCAGAGAGAAAACGTGGTGTCCCTAATTGACAACA | |
| | AATACAACTGTGCCTCAAACCTGCTGTCCGACGCCCAAATCACCGACGAGGA | |
| | GCTGAGGACCAACAGCAGAAGCATCAAGGATCTGCTCGACGCCGTGAAGAGT | |
| | GCCCAGAGATTCTTCAGACTGCTGTGCGGTTCTGGCAATGAGCCTGATAAAG | |
| | ACCACAGCTTTTATGACGAGTACACCCCTGCTTTCGAGGCCCTGGAAAACAG | |
| | CATCAACCCCCTGTACAACAAGGTCCGCAGCTTCGTGACCAAAAAGGACTTC | |
| | AGCACAGACAAGTTCAAACTGAACTTCGACAGCAGCAGCTTCCTGAGCGGAT | |
| | GGGCCAAGAAAAGCGAGTACGAGAAGAGCAGCGCTTTCATCTTCATCAGGGA | |
| | TAATCAGTACTACCTGGGAATTAATAAGTGCCTGAGTAAAGAGGACATCGCCT | |
| | ACCTGGAGGACAGCACCTCTAGCAGCGACACAAAGAGAGTGGTGTACATGTT | |
| | TCAGAAGGTGGATGCCACCAATATCCCAAGAATCTTCATCAGATCCAAGGGCA | |
| | GCAACCTGGCCCCTGCTGTGAACGAGTTCCAGCTGCCTATCGAAACCATCCT | |
| | GGATATCTACGACAACAAGTTCTTCACCACCAGTTACCAGAAGAAGGATAGAA | |
| | CCAAATGGAAGGAAAGCCTGACCAAGCTGATCGACTACTACAAGCTGGGCTT | |
| | TAGCCAGCACAAGTCCTATGCCGATTTCGATTTAAAGTGGAAAGCCAGCTCAG | |
| | AATACAATGACATCAATGATTTCCTGGCCGACGTGCAGAGATTCTGCTACAGA | |
| | ATTGAGTTCATCAATATCAATTGGGACAAGCTCATCGAGTTCACAGAGGACGG | |
| | CAAGTTCTACCTGTTTAGAATCGCCAACAAAGACCTGTCTGGCAACAGCACTG | |
| | GCCTGCCCAATCTGCACACCATCTACTGGAAGATGCTGTTCGACGAGAGCAA | |
| | CCTGAAGGACATCGTGTACAAGCTGAGCGGCAACGCTGAGGTGTTTATGCGC | |
| | TACAACAGCCTGAAGAACCCCATTGTGCACAAGGCCGGAGTGGAAATCAAGA | |
| | ATAAGTGTCCTTTCACCGAGAAGAAAACCAGCATCTTTGACTACGACATTATC | |
| | AAGGACCGCAGATACACCAAGGACCAGCTGGAACTGCATGTGCCTATCCTGA | |
| | TGAACTTCAAGTCTCCATCTGCCGCTAAAGGCAAAGCCTTTAACAAGGAGTGC | |
| | CTGGAATACATCAGAAACAACGGCATCAAGCACATCATCGGCATCGACAGAG | |
| | GAGAGCGGAATCTGCTTTACATGGTGATCACAGACCTGGACGGCAACATCGT | |
| | GGAACAGAAGTCTCTGAACCAGATCGCCTCCAATCCAAAGCTGCCTCTGTTCA | |
| | GACAGGACTACAACAAGCTGCTGAAAACCAAAGCTGACGCCAACGCACAAGC | |
| | CAGAAGAGACTGGGAGACAATAGACACCGTGAAGGAGATTAAGTTCGGCTTC | |
| | CTGAGCCAGATCGTGCACGAGATCGCTATGGCCATCATCAAGTACGACGCCA | |
| | TTGTGGTCCTGGAAAACCTGAACAGAGGCTTCATGCAAAAACGGGGCCTGGA | |
| | AAACAACGTGTATCAGAAGTTCGAGCAAATGCTCCTCGATAAACTGAGCTACT | |
| | ATGTCGACAAGACCAAACACCCTGAGGAAGCTGGCGGAGCCCTGCACGCCT | |
| | ATCAGTTAAGCGATACCTACGCCAACTTCAATTCCTTGAGCAAGAACGCTATG | |
| | GTGAGACAGTCTGGCTTCGTGTTCTACATCCCCGCCTGGCTGACCAGCAAGA | |
| | TCGATCCTGTGACCGGCTTCGCCTCTTTCCTGAAGTTCCACAGAGATGATAGC | |
| | ATGGCCACCATCAAGAGCACCATCTCCAAATTCGACTGCTTCAAGTACGACAA | |
| | GGAATGCGACATGTTCCACATCAGAATAGATTACAACAAATTTAGCACTTCAT | |
| | GCAGCGGTGGCCAGCGGAAGTGGGATCTGTTCACATTCGGAGACAGAATCCT | |
| | GGCCGAGAGAAACACCATGCAGAACAGTAGATACGTTTACCAGACAGTTAAC | |
| | CTGACCTCTGAGTTCAAGAACCTGTTCGCCACAAAGGATATCGATATAAGCGG | |
| | GAACCTGAAGGATAGCATCTGCAAGATCGAGGACGTGGGCTTCTTCCGGAAG | |
| | CTGAGCCAGCTGCTGAGCCTGACACTACAGCTTCGGAACAGCAACGCTGAAA | |
| | CCGGAGAAGATTTCCTGATCAGCCCTGTGGCCGACAAGGACGGCAACTTCTT | |
| | TGACAGCAGAAACTGCCCCGACAGCCTGCCAAAGGATGCAGACGCGAATGG | |
| | CGCTTATAACATTGCCAGGAAGGGCCTGATGCTGGTGGAGCAACTGAAGCGG | |
| | TGCAAGGACGTGAGCAAGTTCAAGCCTGCTATCAAGAACGAGGACTGGCTGG | |
| | ACTACGTGCAGCGG | |
| Expression | ATGggcGAAATCAGTAATCGGTTTACAAACAAGTACCAGGTGTCTAAGACCCTG | 24 |
| construct (with | CGGTTCAGACTGGAGCCTACAGGCGGGACCGATGACCTGCTGTGCCAGGCC | |
| N-terminal | CAGATCATCGAGGGCGATGAGCGGCGCAACAAAGAAGCCATCACCATGAAAC | |
| methionine | AGATCCTCGACAACTGTCACAAGCAGATCATCGAAAGAGTGCTGTCCGACTTC | |
| and stop | AACTTCAAAGAGCACTCCCTGGAAGAGTTCTTTAAGGTGTACACACGGAACGA | |
| codon, | CGATGACAGAGAGAAGGATATCGAGAACCTGCAGGCAAAGATGCGCAAGGAA | |
| includes V5- | ATCGCCGCCGCCTTTACTAAGCAAGACGTGACAAAACTGTTTTCTTCCAAGTT | |
| tag and C- | TAAAGACTTTGTCGAAAGGGGTCTGATCAAGTACGCCAGCAACGAGAAGGAG | |
| terminal NLS) | CGGAATATCGTGTCCCGGTTCAAGGGCTTTGCCACATACTTCACCGGCTTCAA | |
| | CACAAACCGCCTGAACATGTACAGCGAGGAAGCCAAATCTACGGCCATTAGC | |
| | TTCCGGCTGATCAACCAGAACCTCATCAAATTCATCGACAATATCCTGGTGTA | |
| | CAAGAAGGTGTCTCAGACCCTCCCTTCTGATGTCCTGAGCAACATCTACATCG | |
| | ACTTCAAGGCCATCATCAATACCAGCAGCCTGGAGGAGTTCTTCTCCATCAAC | |
| | AACTACAACAACATCCTGACCCAGAAGCAGATCGAGATCTTCAACGCTGTGAT | |
| | CGGCGGAAAGAAGGATAAGGATGAGAAAATTATCACAAAGGGCTTCAACCAG | |
| | TACATCAATGAATATAATCAGACCAACAAGAATATCAGACTGCCAAAGATGAT | |
| | GAGACTGTTCAATCAGATACTGAGCGACCGGGAAGGCGTGTCAGCTAGACCT | |
| | GAGCCCTTCAACAACGCCAACGAGACAATCAGCTCCGTGAGAGACTGTTTTA | |
| | CAAACGAAATCAGCAAGCAGATCACCATCCTGTCTGAAACCACCAGTAAGATC | |
| | GAGAGCTTCGACATCGATAGAATCTACATCAAGGGCGGAGAGGACCTGCGG | |
| | GCCCTGAGCAACAGCATCTACGGCTACTTCAACTACATCCACGATAGAATCGC | |
| | TGATAAGTGGAAGCACAACAATCCTCAGGGCAAGAAGAGCCCCGAGAGCTAC | |
| | CAAAAGAATCTGAACGCCTACCTGAAGGGCATAAAGAGCGTGAGCCTGCATT | |
| | CTATCGCCAACATCTGTGGCGACAACAAGGTGATCGAATATTTTAGAAATCTC | |
| | GGCGCCGAGAACACAGTGGATTTTCAGAGAGAAAACGTGGTGTCCCTAATTG | |
| | ACAACAAATACAACTGTGCCTCAAACCTGCTGTCCGACGCCCAAATCACCGAC | |
| | GAGGAGCTGAGGACCAACAGCAGAAGCATCAAGGATCTGCTCGACGCCGTG | |
| | AAGAGTGCCCAGAGATTCTTCAGACTGCTGTGCGGTTCTGGCAATGAGCCTG | |
| | ATAAAGACCACAGCTTTTATGACGAGTACACCCCTGCTTTCGAGGCCCTGGAA | |
| | AACAGCATCAACCCCCTGTACAACAAGGTCCGCAGCTTCGTGACCAAAAAGG | |
| | ACTTCAGCACAGACAAGTTCAAACTGAACTTCGACAGCAGCAGCTTCCTGAGC | |
| | GGATGGGCCAAGAAAAGCGAGTACGAGAAGAGCAGCGCTTTCATCTTCATCA | |
| | GGGATAATCAGTACTACCTGGGAATTAATAAGTGCCTGAGTAAAGAGGACATC | |
| | GCCTACCTGGAGGACAGCACCTCTAGCAGCGACACAAAGAGAGTGGTGTACA | |
| | TGTTTCAGAAGGTGGATGCCACCAATATCCCAAGAATCTTCATCAGATCCAAG | |
| | GGCAGCAACCTGGCCCCTGCTGTGAACGAGTTCCAGCTGCCTATCGAAACCA | |
| | TCCTGGATATCTACGACAACAAGTTCTTCACCACCAGTTACCAGAAGAAGGAT | |
| | AGAACCAAATGGAAGGAAAGCCTGACCAAGCTGATCGACTACTACAAGCTGG | |
| | GCTTTAGCCAGCACAAGTCCTATGCCGATTTCGATTTAAAGTGGAAAGCCAGC | |
| | TCAGAATACAATGACATCAATGATTTCCTGGCCGACGTGCAGAGATTCTGCTA | |
| | CAGAATTGAGTTCATCAATATCAATTGGGACAAGCTCATCGAGTTCACAGAGG | |
| | ACGGCAAGTTCTACCTGTTTAGAATCGCCAACAAAGACCTGTCTGGCAACAGC | |
| | ACTGGCCTGCCCAATCTGCACACCATCTACTGGAAGATGCTGTTCGACGAGA | |
| | GCAACCTGAAGGACATCGTGTACAAGCTGAGCGGCAACGCTGAGGTGTTTAT | |
| | GCGCTACAACAGCCTGAAGAACCCCATTGTGCACAAGGCCGGAGTGGAAATC | |
| | AAGAATAAGTGTCCTTTCACCGAGAAGAAAACCAGCATCTTTGACTACGACAT | |
| | TATCAAGGACCGCAGATACACCAAGGACCAGCTGGAACTGCATGTGCCTATC | |
| | CTGATGAACTTCAAGTCTCCATCTGCCGCTAAAGGCAAAGCCTTTAACAAGGA | |
| | GTGCCTGGAATACATCAGAAACAACGGCATCAAGCACATCATCGGCATCGAC | |
| | AGAGGAGAGCGGAATCTGCTTTACATGGTGATCACAGACCTGGACGGCAACA | |
| | TCGTGGAACAGAAGTCTCTGAACCAGATCGCCTCCAATCCAAAGCTGCCTCT | |
| | GTTCAGACAGGACTACAACAAGCTGCTGAAAACCAAAGCTGACGCCAACGCA | |
| | CAAGCCAGAAGAGACTGGGAGACAATAGACACCGTGAAGGAGATTAAGTTCG | |
| | GCTTCCTGAGCCAGATCGTGCACGAGATCGCTATGGCCATCATCAAGTACGA | |
| | CGCCATTGTGGTCCTGGAAAACCTGAACAGAGGCTTCATGCAAAAACGGGGC | |
| | CTGGAAAACAACGTGTATCAGAAGTTCGAGCAAATGCTCCTCGATAAACTGAG | |
| | CTACTATGTCGACAAGACCAAACACCCTGAGGAAGCTGGCGGAGCCCTGCAC | |
| | GCCTATCAGTTAAGCGATACCTACGCCAACTTCAATTCCTTGAGCAAGAACGC | |
| | TATGGTGAGACAGTCTGGCTTCGTGTTCTACATCCCCGCCTGGCTGACCAGC | |
| | AAGATCGATCCTGTGACCGGCTTCGCCTCTTTCCTGAAGTTCCACAGAGATGA | |
| | TAGCATGGCCACCATCAAGAGCACCATCTCCAAATTCGACTGCTTCAAGTACG | |
| | ACAAGGAATGCGACATGTTCCACATCAGAATAGATTACAACAAATTTAGCACTT | |
| | CATGCAGCGGTGGCCAGCGGAAGTGGGATCTGTTCACATTCGGAGACAGAAT | |
| | CCTGGCCGAGAGAAACACCATGCAGAACAGTAGATACGTTTACCAGACAGTT | |
| | AACCTGACCTCTGAGTTCAAGAACCTGTTCGCCACAAAGGATATCGATATAAG | |
| | CGGGAACCTGAAGGATAGCATCTGCAAGATCGAGGACGTGGGCTTCTTCCGG | |
| | AAGCTGAGCCAGCTGCTGAGCCTGACACTACAGCTTCGGAACAGCAACGCTG | |
| | AAACCGGAGAAGATTTCCTGATCAGCCCTGTGGCCGACAAGGACGGCAACTT | |
| | CTTTGACAGCAGAAACTGCCCCGACAGCCTGCCAAAGGATGCAGACGCGAAT | |
| | GGCGCTTATAACATTGCCAGGAAGGGCCTGATGCTGGTGGAGCAACTGAAGC | |
| | GGTGCAAGGACGTGAGCAAGTTCAAGCCTGCTATCAAGAACGAGGACTGGCT | |
| | GGACTACGTGCAGCGGtctagaAAGCGGACAGCAGACGGCTCCGAATTTGAAA | |
| | GCCCTAAGAAAAAGAGAAAGGTGggatccGGCAAACCTATCCCCAATCCCCTGC | |
| | TGGGCCTGGACAGCACCTGA | |

In some embodiments a ZZFT Type V Cas protein comprises an amino acid sequence of SEQ ID NO: 19, SEQ ID NO:20, or SEQ ID NO:21. In some embodiments, a ZZFT Type V Cas protein has nickase activity, for example resulting from one or more amino acid substitutions relative to the sequence of SEQ ID NO: 19, SEQ ID NO:20, or SEQ ID NO:21. In some embodiments, the one or more amino acid substitutions providing nickase activity comprise a D856 substitution, wherein the position of the D856 substitution is defined with respect to the amino acid numbering of SEQ ID NO:20 (corresponding to amino acid 908 of SEQ ID NO: 121). In some embodiments, the one or more amino acid substitutions providing nickase activity comprise an E949 substitution, wherein the position of the E949 substitution is defined with respect to the amino acid numbering of SEQ ID NO:20 (corresponding to amino acid 993 of SEQ ID NO: 121). In some embodiments, the one or more amino acid substitutions providing nickase activity comprise a R1166 substitution, wherein the position of the R1166 substitution is defined with respect to the amino acid numbering of SEQ ID NO:20 (corresponding to amino acid 1226 of SEQ ID NO: 121). In some embodiments, the one or more amino acid substitutions providing nickase activity comprise a D1203 substitution, wherein the position of the D1203 substitution is defined with respect to the amino acid numbering of SEQ ID NO:20 (corresponding to amino acid 1263 of SEQ ID NO: 121). In some embodiments, a ZZFT Type V Cas protein is catalytically inactive, for example due to a R1166 substitution in combination with a D856 substitution, a E949 substitution, and/or D1203 substitution.

### 6.2.5. YYAN Type V Cas Proteins

In one aspect, the disclosure provides YYAN Type V Cas proteins. YYAN Type V Cas proteins can be further classified as Type V-A Cas proteins. The YYAN Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:25. In some embodiments, the YYAN Type V Cas proteins comprise an amino acid sequence that is at least 95%, at least 96%, at least 97%, at least 98%, or at least 99% identical to SEQ ID NO:25. In some embodiments, a YYAN Type V Cas protein comprises an amino acid sequence that is identical to SEQ ID NO:25.

Exemplary YYAN Type V Cas protein sequences and nucleotide sequences encoding exemplary YYAN Type V Cas proteins are set forth in Table 1E.

TABLE 1E
YYAN Type V Cas Sequences

| | | SEQ ID |
|---|---|---|
| Name | Sequence | NO. |
| Wildtype | KINAFINCYSMSKTLRFKLAPEYETEKNLLEKGFLDRDKLRADDYDLMKKVIDKYHKH | 25 |
| amino acid | FIDKALEGFKFDLLQEYAEAFYSQSADDDGKKLEEIKKKMCKELATCFSKQDEFKLLD | |
| sequence | KKELVEKLIPAAEFIEDEEKDIAKRFKGFTTYFTGFNENRQNLYAAELKHGTIAFRLIEE | |
| (without N- | NLPAFLYNCKKGVKIFEGLDAVDAETLNNELGEILSIENVKDVLSVEYYNKTLTQNGID | |
| terminal | VYNRIIGGYTQEDGTKIKGVNEYVNLYNQTHDKKLPSLAKLKKQILSDSYSLSFLPAKF | |
| methionine) | NDDSELLLSLKKFYSTVNEETGLSVEKAIQEMRDVFSHIDDCDLHNVFIDAKFINKVSN | |
| | DVFGNWSVLIDGINAEYEKLNPFNGKNLDNYEEKRKAFLNKIESYSVDALQAYSGKEE | |
| | KIADYVQKRAVELYDSVACAYENMSNKVINAREGKVKLYQDDEKTEIIKTFLDAVQEF | |
| | KKFAEMFCYDGTDGDTTFYGEFANYYGQIAEIIPLYNKCRNYLTKKPYSEDKIKINFDN | |
| | AELLHGWDANKEKNYLTVLLFKNGSYYLGILDKKHKNVLIKDVPEKTQEEPCFKKMIY | |
| | KLLPDPKRNMPRIILHAKSNKKLFEPSDEIYRIYETESFKTDIDDCHRLIDFYKESISKYE | |
| | DWKTFGFKFKETSEYKNIGQFYNEVKEQGYKISFTDIPESYVKDLVNDGKLYLFRLAN | |
| | KDFSPYSKGKKNLHTMYFEGIFDPENIKEKVYALNGGGELFFRCASLNYDKPTHPKN | |
| | VPIKNKTYDFRTDNAKKETSTFEYDLIKDKRYTKDQYTLHCPVTLNFKERGIERINDLV | |
| | RQSLRESDDNYVIGIDRGERNLIYISVIDGKGKIVEQFSMNNLLSGNDVSIDFHKMLET | |
| | REHERDASRKNWNTIDNIKDLKQGYLSYVVKKICDLVVKYDAIVAMEDLNVGFKHGR | |
| | EKFERQVYQKFEKALVDKMSYIVNKNASPHSDGGLFRAYQLTNKKYNENEKQNGFIF | |
| | YVRAWNTSKIDPTTGFVNMLPLKYQSKEKSKEFFDKFEDIFYDENKDMFGFTFRYDD | |
| | FGINIDHKNEWTAYSNGERIITVRNSFGKWDKAKIVLTPAFKKLFDDYNVDCRGDVKR | |
| | QIMNVDDKDFFVRLYKLLSYTMQLRNSDDVDDYILSPVVNAEGKFFDSRNSDGSLPC | |
| | DADANGAYHIAKKAMWAIGKIKEADEESFKKTSLAIDNKTWLEFVQKA | |
| Wildtype | MKINAFINCYSMSKTLRFKLAPEYETEKNLLEKGFLDRDKLRADDYDLMKKVIDKYHK | 26 |
| amino acid | HFIDKALEGFKFDLLQEYAEAFYSQSADDDGKKLEEIKKKMCKELATCFSKQDEFKLL | |
| sequence (with | DKKELVEKLIPAAEFIEDEEKDIAKRFKGFTTYFTGFNENRQNLYAAELKHGTIAFRLIE | |
| N-terminal | ENLPAFLYNCKKGVKIFEGLDAVDAETLNNELGEILSIENVKDVLSVEYYNKTLTQNGI | |
| methionine) | DVYNRIIGGYTQEDGTKIKGVNEYVNLYNQTHDKKLPSLAKLKKQILSDSYSLSFLPAK | |
| | FNDDSELLLSLKKFYSTVNEETGLSVEKAIQEMRDVFSHIDDCDLHNVFIDAKFINKVS | |
| | NDVFGNWSVLIDGINAEYEKLNPFNGKNLDNYEEKRKAFLNKIESYSVDALQAYSGK | |
| | EEKIADYVQKRAVELYDSVACAYENMSNKVINAREGKVKLYQDDEKTEIIKTFLDAVQ | |
| | EFKKFAEMFCYDGTDGDTTFYGEFANYYGQIAEIIPLYNKCRNYLTKKPYSEDKIKINF | |
| | DNAELLHGWDANKEKNYLTVLLFKNGSYYLGILDKKHKNVLIKDVPEKTQEEPCFKK | |
| | MIYKLLPDPKRNMPRIILHAKSNKKLFEPSDEIYRIYETESFKTDIDDCHRLIDFYKESIS | |
| | KYEDWKTFGFKFKETSEYKNIGQFYNEVKEQGYKISFTDIPESYVKDLVNDGKLYLFR | |
| | LANKDFSPYSKGKKNLHTMYFEGIFDPENIKEKVYALNGGGELFFRCASLNYDKPTH | |
| | PKNVPIKNKTYDFRTDNAKKETSTFEYDLIKDKRYTKDQYTLHCPVTLNFKERGIERIN | |
| | DLVRQSLRESDDNYVIGIDRGERNLIYISVIDGKGKIVEQFSMNNLLSGNDVSIDFHKM | |
| | LETREHERDASRKNWNTIDNIKDLKQGYLSYVVKKICDLVVKYDAIVAMEDLNVGFKH | |
| | GREKFERQVYQKFEKALVDKMSYIVNKNASPHSDGGLFRAYQLTNKKYNENEKQNG | |
| | FIFYVRAWNTSKIDPTTGFVNMLPLKYQSKEKSKEFFDKFEDIFYDENKDMFGFTFRY | |
| | DDFGINIDHKNEWTAYSNGERIITVRNSFGKWDKAKIVLTPAFKKLFDDYNVDCRGDV | |
| | KRQIMNVDDKDFFVRLYKLLSYTMQLRNSDDVDDYILSPVVNAEGKFFDSRNSDGSL | |
| | PCDADANGAYHIAKKAMWAIGKIKEADEESFKKTSLAIDNKTWLEFVQKA | |
| Expression | MGKINAFINCYSMSKTLRFKLAPEYETEKNLLEKGFLDRDKLRADDYDLMKKVIDKYH | 27 |
| construct (with | KHFIDKALEGFKFDLLQEYAEAFYSQSADDDGKKLEEIKKKMCKELATCFSKQDEFKL | |
| N-terminal | LDKKELVEKLIPAAEFIEDEEKDIAKRFKGFTTYFTGFNENRQNLYAAELKHGTIAFRLI | |
| methionine, | EENLPAFLYNCKKGVKIFEGLDAVDAETLNNELGEILSIENVKDVLSVEYYNKTLTQNG | |
| V5-tag and C- | IDVYNRIIGGYTQEDGTKIKGVNEYVNLYNQTHDKKLPSLAKLKKQILSDSYSLSFLPA | |
| terminal NLS) | KFNDDSELLLSLKKFYSTVNEETGLSVEKAIQEMRDVFSHIDDCDLHNVFIDAKFINKV | |
| aa sequence | SNDVFGNWSVLIDGINAEYEKLNPFNGKNLDNYEEKRKAFLNKIESYSVDALQAYSG | |
| | KEEKIADYVQKRAVELYDSVACAYENMSNKVINAREGKVKLYQDDEKTEIIKTFLDAV | |
| | QEFKKFAEMFCYDGTDGDTTFYGEFANYYGQIAEIIPLYNKCRNYLTKKPYSEDKIKIN | |
| | FDNAELLHGWDANKEKNYLTVLLFKNGSYYLGILDKKHKNVLIKDVPEKTQEEPCFKK | |
| | MIYKLLPDPKRNMPRIILHAKSNKKLFEPSDEIYRIYETESFKTDIDDCHRLIDFYKESIS | |
| | KYEDWKTFGFKFKETSEYKNIGQFYNEVKEQGYKISFTDIPESYVKDLVNDGKLYLFR | |
| | LANKDFSPYSKGKKNLHTMYFEGIFDPENIKEKVYALNGGGELFFRCASLNYDKPTH | |
| | PKNVPIKNKTYDFRTDNAKKETSTFEYDLIKDKRYTKDQYTLHCPVTLNFKERGIERIN | |
| | DLVRQSLRESDDNYVIGIDRGERNLIYISVIDGKGKIVEQFSMNNLLSGNDVSIDFHKM | |
| | LETREHERDASRKNWNTIDNIKDLKQGYLSYVVKKICDLVVKYDAIVAMEDLNVGFKH | |
| | GREKFERQVYQKFEKALVDKMSYIVNKNASPHSDGGLFRAYQLTNKKYNENEKQNG | |
| | FIFYVRAWNTSKIDPTTGFVNMLPLKYQSKEKSKEFFDKFEDIFYDENKDMFGFTFRY | |
| | DDFGINIDHKNEWTAYSNGERIITVRNSFGKWDKAKIVLTPAFKKLFDDYNVDCRGDV | |
| | KRQIMNVDDKDFFVRLYKLLSYTMQLRNSDDVDDYILSPVVNAEGKFFDSRNSDGSL | |
| | PCDADANGAYHIAKKAMWAIGKIKEADEESFKKTSLAIDNKTWLEFVQKASRKRTAD | |
| | GSEFESPKKKRKVGSGKPIPNPLLGLDST | |
| Wildtype | ATGAAAATTAACGCTTTTATCAACTGTTATTCGATGTCCAAGACGTTGCGATTCAA | 28 |
| coding | GCTTGCGCCCGAATACGAGACGGAAAAGAACCTTTTGGAAAAGGGATTTCTTGAT | |
| sequence (with | CGCGACAAATTGCGCGCGGACGATTATGATTTAATGAAAAAAGTTATCGATAAAT | |
| N-terminal | ATCACAAACATTTTATCGATAAAGCGTTGGAAGGTTTCAAATTCGATTTATTGCAA | |
| methionine | GAGTATGCCGAAGCGTTTTATTCGCAATCGGCCGATGACGACGGCAAAAAACTT | |
| and stop | GAAGAAATCAAAAAGAAAATGTGCAAGGAGTTGGCGACTTGTTTTTCGAAACAAG | |
| codon) | ACGAGTTTAAATTACTCGATAAAAAAGAACTGGTCGAAAAACTAATCCCTGCTGCC | |
| | GAATTTATTGAAGACGAAGAAAAAGATATTGCGAAGAGATTCAAGGGGTTTACGA | |
| | CCTATTTCACGGGATTCAACGAAAACAGGCAAAACTTATACGCCGCAGAACTGAA | |
| | ACACGGGACGATTGCGTTCAGATTGATTGAAGAAAATTTGCCTGCATTTTTGTACA | |
| | ACTGCAAAAAGGGAGTAAAAATATTCGAGGGACTCGACGCAGTCGATGCAGAAA | |
| | CGCTTAATAATGAACTTGGAGAGATTCTTTCAATCGAAAACGTAAAAGATGTATTA | |
| | AGCGTAGAGTATTACAATAAAACGCTCACGCAAAACGGCATAGACGTTTACAACC | |
| | GGATTATAGGCGGCTATACACAGGAAGACGGGACGAAAATCAAAGGTGTCAACG | |
| | AGTACGTCAATTTGTATAACCAGACGCACGACAAAAAACTTCCGTCGCTCGCAAA | |
| | ACTCAAAAAACAGATTTTAAGCGACAGTTATTCGTTGTCGTTTTTGCCCGCAAAAT | |
| | TCAACGACGATTCCGAATTGCTTTTATCGCTTAAAAAGTTTTATTCGACGGTAAAC | |
| | GAAGAGACCGGTTTAAGCGTAGAAAAGGCGATACAGGAAATGCGCGACGTTTTT | |
| | TCACACATCGATGACTGTGATTTGCATAACGTTTTTATCGACGCAAAATTTATAAA | |
| | CAAGGTTTCAAACGACGTTTTCGGGAATTGGAGCGTTTTGATTGACGGCATAAAT | |
| | GCGGAATATGAGAAACTCAATCCGTTCAACGGGAAAAACCTCGACAATTATGAGG | |
| | AAAAACGCAAAGCGTTTTTAAACAAGATCGAAAGCTATTCTGTTGACGCGTTGCA | |
| | GGCATATTCGGGTAAAGAAGAAAAAATCGCCGACTACGTTCAAAAACGTGCGGTC | |
| | GAACTTTACGATAGTGTCGCATGCGCATATGAGAATATGAGTAATAAGGTAATAAA | |
| | TGCGCGAGAAGGGAAGGTTAAACTTTATCAGGACGATGAAAAAACCGAAATAATC | |
| | AAAACGTTTTTGGACGCGGTACAGGAATTCAAAAAGTTTGCCGAGATGTTTTGCT | |
| | ATGACGGCACCGACGGCGATACGACGTTTTACGGCGAATTTGCGAATTATTACG | |
| | GACAAATTGCCGAAATTATACCGCTTTACAATAAATGCAGGAACTATTTGACGAAA | |
| | AAGCCGTATTCCGAAGACAAAATCAAAATAAACTTTGACAACGCTGAGCTTTTGCA | |
| | TGGATGGGACGCAAACAAAGAAAAGAATTATCTGACTGTATTATTATTTAAAAACG | |
| | GCAGTTATTATCTCGGTATTCTGGATAAAAAGCATAAGAACGTTTTGATCAAAGAC | |
| | GTGCCCGAAAAGACGCAGGAGGAGCCGTGTTTCAAGAAAATGATTTACAAATTAC | |
| | TCCCTGATCCGAAACGAAATATGCCTAGAATAATATTACATGCAAAAAGTAACAAG | |
| | AAGTTGTTTGAGCCTAGTGATGAGATATATAGGATATATGAAACAGAATCGTTTAA | |
| | AACTGACATTGACGACTGCCATAGGTTGATTGATTTTTATAAAGAAAGTATAAGCA | |
| | AGTACGAGGACTGGAAGACGTTCGGGTTCAAGTTCAAAGAAACGAGCGAGTATA | |
| | AAAACATAGGGCAATTTTATAACGAAGTTAAAGAGCAGGGATATAAGATTTCATTC | |
| | ACGGATATACCCGAAAGTTACGTCAAAGACTTGGTAAACGACGGGAAACTGTATT | |
| | TATTCAGGCTTGCTAATAAAGATTTTTCTCCGTACAGCAAGGGCAAAAAGAATTTG | |
| | CATACGATGTATTTCGAGGGAATATTTGATCCTGAAAACATAAAAGAAAAGGTTTA | |
| | TGCGCTTAACGGCGGCGGCGAGTTGTTTTTCAGATGCGCGAGCTTGAATTACGA | |
| | CAAACCGACGCATCCGAAAAACGTACCGATTAAAAACAAAACGTATGATTTCCGC | |
| | ACCGATAATGCGAAAAAAGAAACAAGCACGTTTGAATACGACCTCATAAAAGATA | |
| | AGCGATATACGAAAGATCAATACACGTTGCATTGTCCGGTGACGCTTAATTTTAA | |
| | GGAAAGAGGAATCGAAAGAATAAACGATCTCGTAAGGCAATCGTTGCGTGAAAGT | |
| | GACGACAACTACGTAATCGGCATTGATCGGGGCGAAAGAAACTTAATTTACATCA | |
| | GTGTTATCGACGGAAAAGGAAAGATTGTCGAGCAATTCTCGATGAACAATTTGTT | |
| | AAGCGGTAACGACGTGTCGATAGATTTCCACAAAATGCTCGAAACGCGGGAGCA | |
| | CGAGCGCGACGCGTCCAGAAAAAACTGGAATACAATCGACAATATCAAAGACTTG | |
| | AAGCAAGGATATTTAAGTTATGTCGTAAAGAAAATTTGCGACCTTGTCGTAAAATA | |
| | CGACGCGATTGTCGCAATGGAAGACTTAAACGTCGGGTTCAAGCACGGACGAGA | |
| | AAAGTTCGAGCGACAGGTATATCAGAAATTTGAAAAAGCACTTGTCGACAAAATG | |
| | AGTTATATCGTAAACAAGAACGCGTCGCCGCATTCCGACGGAGGTTTGTTCAGG | |
| | GCATACCAGCTGACCAATAAAAAGTATAATGAAAACGAAAAACAAAACGGTTTTAT | |
| | TTTCTATGTCAGAGCGTGGAATACCAGTAAGATCGATCCGACGACCGGGTTTGTA | |
| | AACATGCTTCCGTTAAAATATCAGAGCAAAGAAAAATCAAAAGAATTTTTCGATAA | |
| | ATTTGAAGATATTTTTTACGATGAAAACAAGGATATGTTCGGTTTTACATTCAGATA | |
| | TGACGATTTCGGTATAAATATCGATCATAAAAACGAATGGACGGCTTATTCAAACG | |
| | GCGAACGAATAATCACCGTACGAAATTCGTTCGGCAAGTGGGATAAAGCGAAGA | |
| | TCGTATTGACGCCGGCATTTAAGAAACTGTTTGACGACTATAACGTGGATTGTCG | |
| | CGGCGACGTCAAACGACAGATTATGAACGTTGACGACAAAGACTTTTTCGTTAGG | |
| | TTATATAAGCTTTTGTCGTATACGATGCAGTTGAGAAACTCCGACGATGTTGACGA | |
| | CTATATTTTGTCGCCCGTCGTTAATGCGGAAGGGAAGTTCTTTGACAGTCGCAAT | |
| | TCGGACGGCAGTTTGCCTTGCGACGCGGACGCAAACGGAGCGTATCATATTGCC | |
| | AAAAAGGCAATGTGGGCAATCGGGAAGATAAAAGAAGCGGACGAAGAAAGTTTT | |
| | AAAAAGACAAGTCTTGCAATCGACAACAAGACGTGGCTTGAATTCGTTCAAAAGG | |
| | CATAA | |
| Codon | AAGATCAACGCTTTTATCAACTGTTACAGCATGAGCAAGACCCTGAGATTCAAGC | 29 |
| optimized | TGGCCCCTGAGTACGAAACCGAGAAGAACCTGCTGGAAAAGGGCTTTCTGGACC | |
| coding | GGGACAAGCTGAGAGCCGACGACTACGACCTGATGAAGAAGGTGATAGACAAGT | |
| sequence (no | ACCACAAGCACTTCATCGACAAGGCCCTGGAAGGCTTCAAGTTTGACCTGCTGC | |
| N-terminal | AAGAATACGCTGAGGCCTTTTACAGCCAGAGCGCCGACGACGACGGCAAGAAGC | |
| methionine, no | TCGAAGAGATCAAGAAGAAGATGTGCAAGGAGCTGGCCACATGCTTCAGCAAGC | |
| stop codon) | AAGACGAGTTCAAGCTACTGGATAAGAAAGAGCTGGTGGAAAAGCTGATCCCAG | |
| | CCGCTGAGTTCATCGAGGACGAGGAAAAAGACATTGCCAAGAGATTCAAAGGCT | |
| | TTACAACCTACTTTACCGGCTTCAATGAAAACAGACAGAATCTGTACGCCGCCGA | |
| | GCTGAAGCACGGAACAATCGCCTTCAGACTGATCGAGGAGAACTTGCCTGCCTT | |
| | CCTGTACAATTGCAAGAAGGGTGTTAAGATCTTCGAGGGCCTGGACGCTGTGGA | |
| | TGCTGAGACTCTCAACAACGAGCTGGGCGAGATCCTGAGCATCGAAAACGTGAA | |
| | GGACGTGCTGTCCGTGGAGTACTACAACAAAACCCTGACCCAAAACGGCATCGA | |
| | TGTGTACAATAGAATCATCGGCGGCTACACCCAGGAGGATGGCACCAAGATCAA | |
| | GGGAGTGAACGAGTACGTGAACCTGTATAACCAGACACACGACAAGAAACTGCC | |
| | TTCTCTGGCTAAGCTGAAGAAGCAAATCCTGTCTGACTCCTATTCTCTGTCATTCC | |
| | TGCCCGCCAAGTTTAACGACGACTCTGAGCTCCTGCTCAGCCTGAAGAAGTTTTA | |
| | CAGCACCGTGAACGAGGAAACAGGACTGAGCGTGGAGAAAGCTATCCAGGAGAT | |
| | GAGAGATGTGTTCAGCCACATTGACGACTGCGACCTTCACAACGTCTTTATCGAT | |
| | GCCAAGTTCATCAACAAGGTGAGCAACGACGTGTTCGGCAACTGGTCGGTCCTG | |
| | ATCGATGGCATCAATGCCGAGTACGAGAAGCTGAACCCCTTCAACGGCAAGAAC | |
| | CTGGACAACTACGAGGAAAAAAGAAAGGCCTTTCTGAACAAAATCGAGAGCTATA | |
| | GCGTGGACGCCCTGCAGGCCTACAGCGGCAAGGAAGAGAAGATCGCCGATTAT | |
| | GTGCAGAAACGGGCCGTTGAACTGTACGACAGCGTGGCTTGTGCTTACGAAAAC | |
| | ATGAGCAACAAAGTGATCAACGCCCGGGAAGGCAAGGTGAAGCTGTACCAGGAC | |
| | GACGAAAAGACCGAGATTATCAAGACCTTCCTGGATGCTGTTCAGGAGTTCAAGA | |
| | AGTTCGCCGAAATGTTCTGCTACGATGGAACAGATGGAGATACCACCTTCTACGG | |
| | CGAGTTCGCCAATTATTACGGCCAGATCGCCGAGATAATCCCCCTGTACAACAAG | |
| | TGCAGAAACTATCTGACAAAGAAACCTTACAGCGAGGACAAGATTAAGATCAACT | |
| | TCGATAACGCGGAACTGCTGCATGGATGGGACGCCAACAAGGAAAAGAACTACC | |
| | TGACAGTCCTGCTGTTCAAAAATGGATCATATTACCTGGGCATCCTGGATAAAAA | |
| | GCATAAGAACGTGCTGATTAAGGACGTTCCTGAAAAGACACAGGAAGAGCCCTG | |
| | TTTCAAAAAAATGATCTACAAGCTGCTGCCTGATCCCAAGCGGAATATGCCTAGG | |
| | ATCATCTTGCACGCCAAAAGCAATAAAAAACTGTTCGAGCCTAGCGATGAGATCT | |
| | ACAGAATCTATGAGACAGAGAGCTTCAAGACCGACATCGACGATTGCCACAGACT | |
| | GATCGATTTCTACAAGGAATCCATCAGCAAGTACGAGGACTGGAAAACCTTTGGA | |
| | TTTAAATTCAAAGAAACCAGCGAGTACAAGAACATCGGACAGTTCTACAACGAGG | |
| | TGAAGGAACAGGGCTACAAGATTAGCTTCACCGACATCCCTGAGAGCTACGTGA | |
| | AGGATCTGGTGAATGATGGCAAGCTGTATCTGTTTAGACTCGCCAACAAGGATTT | |
| | CTCTCCATACTCCAAGGGCAAAAAGAACCTGCACACCATGTACTTCGAGGGAATC | |
| | TTCGACCCCGAAAACATCAAGGAGAAAGTGTACGCCCTGAACGGCGGCGGCGA | |
| | GCTGTTCTTCCGCTGTGCCTCTCTGAACTACGACAAGCCTACCCACCCCAAGAAC | |
| | GTGCCTATCAAGAACAAGACCTACGATTTTAGAACCGATAACGCTAAGAAAGAAA | |
| | CCAGTACATTCGAGTACGACCTGATCAAAGATAAACGGTACACAAAGGACCAGTA | |
| | CACACTGCACTGCCCTGTGACACTGAATTTCAAGGAGCGTGGAATCGAACGCAT | |
| | CAACGACCTGGTGCGGCAGAGCCTGCGGGAAAGCGACGACAACTACGTCATCG | |
| | GCATCGACAGAGGGGAGAGAAATCTGATCTACATCTCTGTGATCGACGGCAAGG | |
| | GCAAGATCGTCGAGCAGTTCAGCATGAACAACCTGCTGTCCGGCAACGACGTCA | |
| | GCATCGACTTCCACAAGATGCTGGAAACCAGAGAGCACGAGCGGGACGCCTCCA | |
| | GAAAGAACTGGAACACCATCGACAACATCAAGGACCTGAAGCAGGGCTACCTGA | |
| | GTTACGTGGTGAAAAAGATCTGCGACCTGGTCGTGAAGTATGATGCCATCGTGG | |
| | CTATGGAGGATCTGAACGTGGGCTTTAAACACGGCAGAGAGAAGTTCGAGAGAC | |
| | AGGTGTACCAGAAGTTTGAGAAAGCCCTGGTGGACAAGATGAGCTACATCGTGA | |
| | ATAAAAATGCTAGTCCTCACAGCGATGGCGGCCTGTTCAGAGCTTATCAGCTGAC | |
| | CAACAAGAAATACAACGAGAATGAAAAGCAGAACGGATTCATCTTTTACGTGAGA | |
| | GCCTGGAATACCAGCAAGATCGACCCAACAACAGGCTTCGTGAACATGTTGCCA | |
| | CTGAAATACCAATCTAAGGAAAAGTCCAAGGAGTTCTTCGACAAGTTCGAGGATA | |
| | TCTTCTATGATGAAAACAAAGACATGTTCGGCTTCACCTTCCGGTACGACGACTT | |
| | CGGCATCAACATCGACCACAAGAATGAATGGACCGCCTACAGCAATGGTGAGCG | |
| | GATCATCACCGTGCGGAACAGCTTCGGCAAATGGGATAAAGCGAAGATCGTGCT | |
| | GACCCCTGCTTTTAAGAAGCTGTTCGATGATTACAACGTGGACTGCAGAGGCGA | |
| | CGTGAAGCGACAGATTATGAACGTGGACGACAAAGATTTCTTCGTGCGGCTGTA | |
| | CAAGCTGCTGAGCTACACCATGCAGCTGAGAAACAGCGACGACGTGGACGATTA | |
| | CATCCTGAGCCCCGTGGTGAATGCCGAAGGCAAGTTCTTCGACAGCAGAAACTC | |
| | TGACGGCTCTCTGCCTTGTGACGCCGATGCCAACGGCGCCTACCACATCGCCAA | |
| | GAAGGCCATGTGGGCCATCGGCAAGATCAAGGAAGCCGATGAGGAATCTTTTAA | |
| | GAAAACCTCCCTCGCCATCGACAACAAAACCTGGCTGGAGTTCGTGCAGAAAGC | |
| | C | |
| Expression | ATGggcAAGATCAACGCTTTTATCAACTGTTACAGCATGAGCAAGACCCTGAGATT | 30 |
| construct (with | CAAGCTGGCCCCTGAGTACGAAACCGAGAAGAACCTGCTGGAAAAGGGCTTTCT | |
| N-terminal | GGACCGGGACAAGCTGAGAGCCGACGACTACGACCTGATGAAGAAGGTGATAG | |
| methionine | ACAAGTACCACAAGCACTTCATCGACAAGGCCCTGGAAGGCTTCAAGTTTGACCT | |
| and stop | GCTGCAAGAATACGCTGAGGCCTTTTACAGCCAGAGCGCCGACGACGACGGCAA | |
| codon, | GAAGCTCGAAGAGATCAAGAAGAAGATGTGCAAGGAGCTGGCCACATGCTTCAG | |
| includes V5- | CAAGCAAGACGAGTTCAAGCTACTGGATAAGAAAGAGCTGGTGGAAAAGCTGAT | |
| tag and C- | CCCAGCCGCTGAGTTCATCGAGGACGAGGAAAAAGACATTGCCAAGAGATTCAA | |
| terminal NLS) | AGGCTTTACAACCTACTTTACCGGCTTCAATGAAAACAGACAGAATCTGTACGCC | |
| | GCCGAGCTGAAGCACGGAACAATCGCCTTCAGACTGATCGAGGAGAACTTGCCT | |
| | GCCTTCCTGTACAATTGCAAGAAGGGTGTTAAGATCTTCGAGGGCCTGGACGCT | |
| | GTGGATGCTGAGACTCTCAACAACGAGCTGGGCGAGATCCTGAGCATCGAAAAC | |
| | GTGAAGGACGTGCTGTCCGTGGAGTACTACAACAAAACCCTGACCCAAAACGGC | |
| | ATCGATGTGTACAATAGAATCATCGGCGGCTACACCCAGGAGGATGGCACCAAG | |
| | ATCAAGGGAGTGAACGAGTACGTGAACCTGTATAACCAGACACACGACAAGAAA | |
| | CTGCCTTCTCTGGCTAAGCTGAAGAAGCAAATCCTGTCTGACTCCTATTCTCTGT | |
| | CATTCCTGCCCGCCAAGTTTAACGACGACTCTGAGCTCCTGCTCAGCCTGAAGAA | |
| | GTTTTACAGCACCGTGAACGAGGAAACAGGACTGAGCGTGGAGAAAGCTATCCA | |
| | GGAGATGAGAGATGTGTTCAGCCACATTGACGACTGCGACCTTCACAACGTCTTT | |
| | ATCGATGCCAAGTTCATCAACAAGGTGAGCAACGACGTGTTCGGCAACTGGTCG | |
| | GTCCTGATCGATGGCATCAATGCCGAGTACGAGAAGCTGAACCCCTTCAACGGC | |
| | AAGAACCTGGACAACTACGAGGAAAAAAGAAAGGCCTTTCTGAACAAAATCGAGA | |
| | GCTATAGCGTGGACGCCCTGCAGGCCTACAGCGGCAAGGAAGAGAAGATCGCC | |
| | GATTATGTGCAGAAACGGGCCGTTGAACTGTACGACAGCGTGGCTTGTGCTTAC | |
| | GAAAACATGAGCAACAAAGTGATCAACGCCCGGGAAGGCAAGGTGAAGCTGTAC | |
| | CAGGACGACGAAAAGACCGAGATTATCAAGACCTTCCTGGATGCTGTTCAGGAG | |
| | TTCAAGAAGTTCGCCGAAATGTTCTGCTACGATGGAACAGATGGAGATACCACCT | |
| | TCTACGGCGAGTTCGCCAATTATTACGGCCAGATCGCCGAGATAATCCCCCTGTA | |
| | CAACAAGTGCAGAAACTATCTGACAAAGAAACCTTACAGCGAGGACAAGATTAAG | |
| | ATCAACTTCGATAACGCGGAACTGCTGCATGGATGGGACGCCAACAAGGAAAAG | |
| | AACTACCTGACAGTCCTGCTGTTCAAAAATGGATCATATTACCTGGGCATCCTGG | |
| | ATAAAAAGCATAAGAACGTGCTGATTAAGGACGTTCCTGAAAAGACACAGGAAGA | |
| | GCCCTGTTTCAAAAAAATGATCTACAAGCTGCTGCCTGATCCCAAGCGGAATATG | |
| | CCTAGGATCATCTTGCACGCCAAAAGCAATAAAAAACTGTTCGAGCCTAGCGATG | |
| | AGATCTACAGAATCTATGAGACAGAGAGCTTCAAGACCGACATCGACGATTGCCA | |
| | CAGACTGATCGATTTCTACAAGGAATCCATCAGCAAGTACGAGGACTGGAAAACC | |
| | TTTGGATTTAAATTCAAAGAAACCAGCGAGTACAAGAACATCGGACAGTTCTACAA | |
| | CGAGGTGAAGGAACAGGGCTACAAGATTAGCTTCACCGACATCCCTGAGAGCTA | |
| | CGTGAAGGATCTGGTGAATGATGGCAAGCTGTATCTGTTTAGACTCGCCAACAAG | |
| | GATTTCTCTCCATACTCCAAGGGCAAAAAGAACCTGCACACCATGTACTTCGAGG | |
| | GAATCTTCGACCCCGAAAACATCAAGGAGAAAGTGTACGCCCTGAACGGCGGCG | |
| | GCGAGCTGTTCTTCCGCTGTGCCTCTCTGAACTACGACAAGCCTACCCACCCCA | |
| | AGAACGTGCCTATCAAGAACAAGACCTACGATTTTAGAACCGATAACGCTAAGAA | |
| | AGAAACCAGTACATTCGAGTACGACCTGATCAAAGATAAACGGTACACAAAGGAC | |
| | CAGTACACACTGCACTGCCCTGTGACACTGAATTTCAAGGAGCGTGGAATCGAA | |
| | CGCATCAACGACCTGGTGCGGCAGAGCCTGCGGGAAAGCGACGACAACTACGT | |
| | CATCGGCATCGACAGAGGGGAGAGAAATCTGATCTACATCTCTGTGATCGACGG | |
| | CAAGGGCAAGATCGTCGAGCAGTTCAGCATGAACAACCTGCTGTCCGGCAACGA | |
| | CGTCAGCATCGACTTCCACAAGATGCTGGAAACCAGAGAGCACGAGCGGGACG | |
| | CCTCCAGAAAGAACTGGAACACCATCGACAACATCAAGGACCTGAAGCAGGGCT | |
| | ACCTGAGTTACGTGGTGAAAAAGATCTGCGACCTGGTCGTGAAGTATGATGCCAT | |
| | CGTGGCTATGGAGGATCTGAACGTGGGCTTTAAACACGGCAGAGAGAAGTTCGA | |
| | GAGACAGGTGTACCAGAAGTTTGAGAAAGCCCTGGTGGACAAGATGAGCTACAT | |
| | CGTGAATAAAAATGCTAGTCCTCACAGCGATGGCGGCCTGTTCAGAGCTTATCAG | |
| | CTGACCAACAAGAAATACAACGAGAATGAAAAGCAGAACGGATTCATCTTTTACG | |
| | TGAGAGCCTGGAATACCAGCAAGATCGACCCAACAACAGGCTTCGTGAACATGT | |
| | TGCCACTGAAATACCAATCTAAGGAAAAGTCCAAGGAGTTCTTCGACAAGTTCGA | |
| | GGATATCTTCTATGATGAAAACAAAGACATGTTCGGCTTCACCTTCCGGTACGAC | |
| | GACTTCGGCATCAACATCGACCACAAGAATGAATGGACCGCCTACAGCAATGGT | |
| | GAGCGGATCATCACCGTGCGGAACAGCTTCGGCAAATGGGATAAAGCGAAGATC | |
| | GTGCTGACCCCTGCTTTTAAGAAGCTGTTCGATGATTACAACGTGGACTGCAGAG | |
| | GCGACGTGAAGCGACAGATTATGAACGTGGACGACAAAGATTTCTTCGTGCGGC | |
| | TGTACAAGCTGCTGAGCTACACCATGCAGCTGAGAAACAGCGACGACGTGGACG | |
| | ATTACATCCTGAGCCCCGTGGTGAATGCCGAAGGCAAGTTCTTCGACAGCAGAA | |
| | ACTCTGACGGCTCTCTGCCTTGTGACGCCGATGCCAACGGCGCCTACCACATCG | |
| | CCAAGAAGGCCATGTGGGCCATCGGCAAGATCAAGGAAGCCGATGAGGAATCTT | |
| | TTAAGAAAACCTCCCTCGCCATCGACAACAAAACCTGGCTGGAGTTCGTGCAGAA | |
| | AGCCtctagaAAGCGGACAGCAGACGGCTCCGAATTTGAAAGCCCTAAGAAAAAGA | |
| | GAAAGGTGggatccGGCAAACCTATCCCCAATCCCCTGCTGGGCCTGGACAGCAC | |
| | CTGA | |

In some embodiments a YYAN Type V Cas protein comprises an amino acid sequence of SEQ ID NO: 25, SEQ ID NO:26, or SEQ ID NO:27. In some embodiments, a YYAN Type V Cas protein has nickase activity, for example resulting from one or more amino acid substitutions relative to the sequence of SEQ ID NO:25, SEQ ID NO:26, or SEQ ID NO:27. In some embodiments, the one or more amino acid substitutions providing nickase activity comprise a D838 substitution, wherein the position of the D838 substitution is defined with respect to the amino acid numbering of SEQ ID NO:26 (corresponding to amino acid 908 of SEQ ID NO: 121). In some embodiments, the one or more amino acid substitutions providing nickase activity comprise an E928 substitution, wherein the position of the E928 substitution is defined with respect to the amino acid numbering of SEQ ID NO:26 (corresponding to amino acid 993 of SEQ ID NO: 121). In some embodiments, the one or more amino acid substitutions providing nickase activity comprise a R1135 substitution, wherein the position of the R1135 substitution is defined with respect to the amino acid numbering of SEQ ID NO:26 (corresponding to amino acid 1226 of SEQ ID NO: 121). In some embodiments, the one or more amino acid substitutions providing nickase activity comprise a D1170 substitution, wherein the position of the D1170 substitution is defined with respect to the amino acid numbering of SEQ ID NO:26 (corresponding to amino acid 1263 of SEQ ID NO:121). In some embodiments, a YYAN Type V Cas protein is catalytically inactive, for example due to a R1135 substitution in combination with a D838 substitution, a E928 substitution, and/or D1170 substitution.

### 6.2.6. ZZGY Type V Cas Proteins

In one aspect, the disclosure provides ZZGY Type V Cas proteins. ZZGY Type V Cas proteins can be further classified as Type V-A Cas proteins. The ZZGY Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:31. In some embodiments, the ZZGY Type V Cas proteins comprise an amino acid sequence that is at least 95%, at least 96%, at least 97%, at least 98%, or at least 99% identical to SEQ ID NO:31. In some embodiments, a ZZGY Type V Cas protein comprises an amino acid sequence that is identical to SEQ ID NO:31.

Exemplary ZZGY Type V Cas protein sequences and nucleotide sequences encoding exemplary ZZGY Type V Cas proteins are set forth in Table 1F.

TABLE 1F
ZZGY Type V Cas Sequences

| | | SEQ |
|---|---|---|
| | | ID |
| Name | Sequence | NO. |
| Wildtype | SKLSTFNEHFQKTLTLRNELVPVGKTLENIISSNVLINDEKRSEDYKKAKEIIDSYHREFI | 31 |
| amino acid | EKSLSSVNVDWNDLYSYLSKKEPEDYAQKQKFLEELENILLEKRKIIVKQFEQYVFGS | |
| sequence | YTDSKGKKTKDLKFENLFKSELFDYLLPNFLKNDEDKKVIGSFNKFTSYFTGFYENRK | |
| (without N- | NLYKSEPLPTAVAYRIVNENFPKFISNKNIFRVWKDNVPQFIEIAKTKLREEGISDLNIEL | |
| terminal | KFDLTNFNSCLNQTGIDTYNDLIGQLNFAINLECQKDKNLCDLLRKKRSLKMVPLYKQI | |
| methionine) | LSDNDSSFSIDEFDNDESAIKDVISFYKKMIGENCPQRTLSELLHGLSSHDLEKIFVQG | |
| | KNLNSVSKNLFGGKNWSLLRDAVIEEKSKEKVFKKVIKSNSTADELDKVLSKEEFSISF | |
| | LSKVSGKDLSVEIDKFVKKQDELLVENNIQNWPSSLKNSEEKNLIKAPLDFLLNFYRFA | |
| | QSFSSNNIDKDMSFYADFDESLSSLENVIGLYNKVRNYATKKPYTLEKIKLNFENPNLA | |
| | SGWSESKENDCLSIILLKEKKYFLGIFNKNNKPNFSEGISHSLSSNGCYRKMRYLLFK | |
| | GFNKMLPKCAFTGEVKDHFKESSDDFSLFNKDTFISPLVITKEIFDLACSKEKVKKYQK | |
| | EYEKINRAEYRQSLVKWITFGLKFLSSYKTTTQFDLSNLKRPEEYCDLKEFYEDVDNL | |
| | TYKIEFLNIKEEDVDALVEKGQLYLFEIRNKDFAKNASGTPNLHTLYFKSIFDSKNLEN | |
| | GIVKLNGEAEIFYRKKSLKKDDITVHREGSYLVNKVCVDPNSGKTEQIPDKIYENIYAF | |
| | VNGKSRDLSKEDEVYYAKATIKKATHEIVKDRRFTVDKFFFHCPITINYKSKDKPSKFN | |
| | DKVLDFLRNNKDINIIGIDRGERNLIYVTVINQNGEIIDCKSFNTIKHQSSTVNYDVDYH | |
| | NKLQEREKNRKEEKRSWNSITKIADLKEGYLSAVIHEVSLMMVKYNAIVVMENLNQGF | |
| | KRIRGGIAERSVYQKFEKMLIDKLNYFVIKNENWTNPGGVLNGYQLTNKVSTIKDIGN | |
| | QCGFLFYVPATYTSKIDPSTGFVNLINFNKYKNSEDRRKLICSFDKICFVQNENLFKFSI | |
| | DYGKLCPDSKIAIKKWDVFSYGTRIIKENLTTGHIEENPEYDPTEELKSLLSSRGIEYQK | |
| | GQNLLETIPTSDMTREFWNSLFKIFKAILQMRNSLTNSPIDRLLSPVKGKDGTFFDTDK | |
| | VEGTKFEKLKDADANGAYNIALKGLLVLEKNDSVESNKDLKNVKKISLEDWLKFVQITL | |
| | RD | |
| Wildtype | MSKLSTFNEHFQKTLTLRNELVPVGKTLENIISSNVLINDEKRSEDYKKAKEIIDSYHRE | 32 |
| amino acid | FIEKSLSSVNVDWNDLYSYLSKKEPEDYAQKQKFLEELENILLEKRKIIVKQFEQYVFG | |
| sequence (with | SYTDSKGKKTKDLKFENLFKSELFDYLLPNFLKNDEDKKVIGSFNKFTSYFTGFYENR | |
| N-terminal | KNLYKSEPLPTAVAYRIVNENFPKFISNKNIFRVWKDNVPQFIEIAKTKLREEGISDLNI | |
| methionine) | ELKFDLTNFNSCLNQTGIDTYNDLIGQLNFAINLECQKDKNLCDLLRKKRSLKMVPLYK | |
| | QILSDNDSSFSIDEFDNDESAIKDVISFYKKMIGENCPQRTLSELLHGLSSHDLEKIFVQ | |
| | GKNLNSVSKNLFGGKNWSLLRDAVIEEKSKEKVFKKVIKSNSTADELDKVLSKEEFSI | |
| | SFLSKVSGKDLSVEIDKFVKKQDELLVENNIQNWPSSLKNSEEKNLIKAPLDFLLNFYR | |
| | FAQSFSSNNIDKDMSFYADFDESLSSLENVIGLYNKVRNYATKKPYTLEKIKLNFENP | |
| | NLASGWSESKENDCLSIILLKEKKYFLGIFNKNNKPNFSEGISHSLSSNGCYRKMRYL | |
| | LFKGFNKMLPKCAFTGEVKDHFKESSDDFSLFNKDTFISPLVITKEIFDLACSKEKVKK | |
| | YQKEYEKINRAEYRQSLVKWITFGLKFLSSYKTTTQFDLSNLKRPEEYCDLKEFYEDV | |
| | DNLTYKIEFLNIKEEDVDALVEKGQLYLFEIRNKDFAKNASGTPNLHTLYFKSIFDSKNL | |
| | ENGIVKLNGEAEIFYRKKSLKKDDITVHREGSYLVNKVCVDPNSGKTEQIPDKIYENIY | |
| | AFVNGKSRDLSKEDEVYYAKATIKKATHEIVKDRRFTVDKFFFHCPITINYKSKDKPSK | |
| | FNDKVLDFLRNNKDINIIGIDRGERNLIYVTVINQNGEIIDCKSFNTIKHQSSTVNYDVDY | |
| | HNKLQEREKNRKEEKRSWNSITKIADLKEGYLSAVIHEVSLMMVKYNAIVVMENLNQ | |
| | GFKRIRGGIAERSVYQKFEKMLIDKLNYFVIKNENWTNPGGVLNGYQLTNKVSTIKDIG | |
| | NQCGFLFYVPATYTSKIDPSTGFVNLINFNKYKNSEDRRKLICSFDKICFVQNENLFKF | |
| | SIDYGKLCPDSKIAIKKWDVFSYGTRIIKENLTTGHIEENPEYDPTEELKSLLSSRGIEY | |
| | QKGQNLLETIPTSDMTREFWNSLFKIFKAILQMRNSLTNSPIDRLLSPVKGKDGTFFDT | |
| | DKVEGTKFEKLKDADANGAYNIALKGLLVLEKNDSVESNKDLKNVKKISLEDWLKFVQ | |
| | ITLRD | |
| Expression | MGSKLSTFNEHFQKTLTLRNELVPVGKTLENIISSNVLINDEKRSEDYKKAKEIIDSYHR | 33 |
| construct (with | EFIEKSLSSVNVDWNDLYSYLSKKEPEDYAQKQKFLEELENILLEKRKIIVKQFEQYVF | |
| N-terminal | GSYTDSKGKKTKDLKFENLFKSELFDYLLPNFLKNDEDKKVIGSFNKFTSYFTGFYEN | |
| methionine, | RKNLYKSEPLPTAVAYRIVNENFPKFISNKNIFRVWKDNVPQFIEIAKTKLREEGISDLN | |
| V5-tag and C- | IELKFDLTNFNSCLNQTGIDTYNDLIGQLNFAINLECQKDKNLCDLLRKKRSLKMVPLY | |
| terminal NLS) | KQILSDNDSSFSIDEFDNDESAIKDVISFYKKMIGENCPQRTLSELLHGLSSHDLEKIFV | |
| aa sequence | QGKNLNSVSKNLFGGKNWSLLRDAVIEEKSKEKVFKKVIKSNSTADELDKVLSKEEFS | |
| | ISFLSKVSGKDLSVEIDKFVKKQDELLVENNIQNWPSSLKNSEEKNLIKAPLDFLLNFY | |
| | RFAQSFSSNNIDKDMSFYADFDESLSSLENVIGLYNKVRNYATKKPYTLEKIKLNFEN | |
| | PNLASGWSESKENDCLSIILLKEKKYFLGIFNKNNKPNFSEGISHSLSSNGCYRKMRY | |
| | LLFKGFNKMLPKCAFTGEVKDHFKESSDDFSLFNKDTFISPLVITKEIFDLACSKEKVK | |
| | KYQKEYEKINRAEYRQSLVKWITFGLKFLSSYKTTTQFDLSNLKRPEEYCDLKEFYED | |
| | VDNLTYKIEFLNIKEEDVDALVEKGQLYLFEIRNKDFAKNASGTPNLHTLYFKSIFDSKN | |
| | LENGIVKLNGEAEIFYRKKSLKKDDITVHREGSYLVNKVCVDPNSGKTEQIPDKIYENI | |
| | YAFVNGKSRDLSKEDEVYYAKATIKKATHEIVKDRRFTVDKFFFHCPITINYKSKDKPS | |
| | KFNDKVLDFLRNNKDINIIGIDRGERNLIYVTVINQNGEIIDCKSFNTIKHQSSTVNYDVD | |
| | YHNKLQEREKNRKEEKRSWNSITKIADLKEGYLSAVIHEVSLMMVKYNAIVVMENLNQ | |
| | GFKRIRGGIAERSVYQKFEKMLIDKLNYFVIKNENWTNPGGVLNGYQLTNKVSTIKDIG | |
| | NQCGFLFYVPATYTSKIDPSTGFVNLINFNKYKNSEDRRKLICSFDKICFVQNENLFKF | |
| | SIDYGKLCPDSKIAIKKWDVFSYGTRIIKENLTTGHIEENPEYDPTEELKSLLSSRGIEY | |
| | QKGQNLLETIPTSDMTREFWNSLFKIFKAILQMRNSLTNSPIDRLLSPVKGKDGTFFDT | |
| | DKVEGTKFEKLKDADANGAYNIALKGLLVLEKNDSVESNKDLKNVKKISLEDWLKFVQ | |
| | ITLRDSRKRTADGSEFESPKKKRKVGSGKPIPNPLLGLDST | |
| Wildtype | ATGTCTAAATTATCAACTTTTAATGAACATTTTCAAAAAACGTTAACTTTAAGAAAC | 34 |
| coding | GAACTAGTTCCTGTAGGAAAAACTCTTGAAAATATCATATCTTCAAATGTATTGATA | |
| sequence (with | AATGATGAGAAAAGAAGTGAAGATTATAAAAAGGCTAAAGAGATCATAGATTCTTA | |
| N-terminal | TCATCGAGAGTTTATAGAGAAATCACTTTCATCAGTAAATGTTGATTGGAATGATC | |
| methionine | TGTACTCGTATTTATCCAAAAAAGAACCAGAAGACTATGCTCAAAAGCAGAAGTTC | |
| and stop | CTCGAAGAGTTAGAAAATATTCTCCTTGAAAAGAGAAAAATTATTGTTAAACAGTT | |
| codon) | TGAGCAATACGTTTTCGGATCATATACAGATTCAAAAGGTAAAAAAACAAAAGATC | |
| | TAAAATTTGAGAATCTTTTTAAATCAGAGTTGTTTGATTATCTTTTGCCAAATTTCC | |
| | TAAAAAATGATGAAGATAAAAAAGTAATAGGTAGTTTTAATAAATTTACATCGTATT | |
| | TTACAGGTTTTTACGAAAATCGAAAGAATTTATATAAATCAGAGCCATTGCCAACA | |
| | GCTGTGGCTTATAGAATAGTTAACGAAAACTTTCCTAAATTCATTTCTAATAAAAAT | |
| | ATCTTTCGCGTGTGGAAAGATAATGTTCCTCAGTTTATAGAAATAGCGAAAACTAA | |
| | ACTAAGAGAAGAAGGCATTTCTGATTTAAATATAGAATTAAAATTTGATTTAACTAA | |
| | TTTCAATTCATGCTTAAATCAAACTGGAATTGATACTTACAATGACTTGATAGGTCA | |
| | ACTCAACTTTGCAATTAACCTTGAATGTCAGAAAGACAAGAATTTATGTGACCTTT | |
| | TAAGGAAGAAAAGAAGCCTTAAAATGGTACCTCTGTATAAACAGATTTTATCTGAT | |
| | AATGATTCTTCATTCAGTATTGATGAATTTGATAATGATGAATCGGCAATAAAAGAT | |
| | GTAATTTCTTTTTATAAGAAAATGATTGGTGAAAATTGTCCTCAACGAACACTATCT | |
| | GAATTGCTACATGGTTTGTCATCTCACGATCTTGAAAAGATATTTGTTCAAGGTAA | |
| | AAACTTAAATTCGGTTTCTAAAAATTTATTTGGAGGGAAGAACTGGTCTTTACTAA | |
| | GGGATGCAGTTATAGAAGAAAAGTCAAAAGAAAAAGTCTTCAAAAAGGTTATAAA | |
| | GTCAAATTCTACCGCAGATGAATTAGACAAAGTTCTTTCCAAGGAAGAATTTTCAA | |
| | TTTCATTCTTATCAAAAGTGAGCGGTAAAGATTTATCAGTAGAAATTGATAAATTTG | |
| | TAAAAAAACAAGACGAACTACTTGTTGAAAATAATATACAAAATTGGCCAAGTTCT | |
| | CTTAAGAACAGCGAAGAGAAAAATCTCATAAAAGCTCCTTTAGATTTCTTACTTAA | |
| | TTTTTATAGATTTGCACAATCATTCTCTTCAAATAATATTGATAAGGATATGTCATTT | |
| | TATGCTGACTTTGATGAATCTCTATCGTCTTTAGAAAATGTAATAGGTCTTTATAAC | |
| | AAAGTCAGAAACTATGCAACTAAGAAACCTTATACACTCGAAAAGATCAAATTGAA | |
| | TTTTGAAAATCCAAATTTAGCTTCTGGATGGAGTGAAAGCAAAGAAAATGATTGTT | |
| | TATCAATTATCTTATTAAAAGAGAAAAAATATTTTTTAGGAATTTTCAACAAAAATAA | |
| | TAAACCTAATTTTTCTGAAGGCATTTCTCATTCACTTTCTTCAAATGGTTGCTACAG | |
| | AAAAATGAGGTATTTATTATTCAAGGGATTCAATAAAATGCTTCCTAAATGTGCTTT | |
| | TACAGGAGAAGTTAAAGATCATTTTAAAGAATCATCGGATGATTTTTCTCTTTTTAA | |
| | CAAGGATACTTTTATCTCTCCTCTTGTAATTACCAAAGAGATCTTTGATTTAGCATG | |
| | TAGTAAAGAAAAGGTAAAAAAATATCAAAAAGAATATGAAAAGATCAATCGTGCTG | |
| | AATATAGACAATCATTGGTTAAGTGGATTACTTTTGGTCTTAAATTTTTGTCATCAT | |
| | ATAAAACTACAACTCAATTTGATTTATCAAATTTAAAAAGACCTGAAGAATACTGCG | |
| | ATCTAAAGGAATTTTATGAAGATGTAGATAATCTTACATACAAGATAGAATTTTTAA | |
| | ATATAAAAGAAGAAGATGTAGATGCATTGGTTGAAAAAGGTCAACTGTATTTATTT | |
| | GAAATTCGAAATAAAGATTTTGCAAAAAATGCAAGTGGCACTCCTAATCTACATAC | |
| | TCTCTATTTTAAAAGTATTTTCGATTCGAAAAATTTAGAGAATGGCATTGTCAAGCT | |
| | TAATGGTGAAGCAGAGATATTTTATAGAAAGAAAAGCTTGAAGAAAGATGACATAA | |
| | CTGTTCATCGAGAAGGCAGTTATCTTGTAAATAAGGTGTGTGTCGATCCTAATTCT | |
| | GGAAAAACAGAACAGATTCCTGACAAAATTTATGAAAATATTTATGCTTTCGTAAA | |
| | TGGTAAATCAAGAGATTTATCTAAGGAGGATGAAGTATATTATGCAAAAGCCACAA | |
| | TAAAAAAAGCTACCCATGAGATCGTAAAAGATAGACGCTTTACTGTAGATAAATTC | |
| | TTTTTCCACTGCCCTATTACTATTAACTATAAATCTAAAGATAAACCTTCAAAATTC | |
| | AATGACAAGGTTTTAGATTTCTTAAGAAATAATAAAGACATCAACATTATAGGCATA | |
| | GATCGAGGAGAGAGAAATCTTATTTATGTAACTGTAATTAATCAAAATGGCGAAAT | |
| | TATTGATTGCAAATCATTTAATACTATCAAACATCAGTCTTCAACAGTGAATTACGA | |
| | TGTTGATTATCACAACAAATTACAAGAAAGAGAAAAAAATAGAAAAGAAGAAAAGA | |
| | GATCTTGGAATAGTATTACTAAAATTGCAGATCTCAAAGAAGGCTATCTTTCTGCT | |
| | GTAATTCATGAAGTTTCATTAATGATGGTTAAGTACAATGCCATTGTCGTTATGGA | |
| | AAATTTGAATCAAGGTTTTAAGAGAATTAGAGGAGGAATTGCTGAAAGATCCGTAT | |
| | ACCAAAAATTTGAAAAGATGCTGATAGATAAACTGAATTATTTTGTTATAAAAAATG | |
| | AAAATTGGACAAATCCTGGTGGGGTCCTCAATGGATATCAGTTAACTAACAAAGT | |
| | GTCTACAATCAAAGATATCGGTAATCAGTGTGGATTTTTATTTTACGTTCCTGCAA | |
| | CTTATACCTCAAAGATTGATCCTTCTACAGGCTTTGTTAATTTAATTAATTTCAATA | |
| | AATATAAAAATTCAGAAGATCGAAGAAAACTCATTTGTAGCTTTGACAAGATATGC | |
| | TTTGTACAGAATGAGAATTTATTTAAATTTTCTATAGATTATGGAAAATTATGCCCA | |
| | GATAGCAAAATTGCTATAAAAAAATGGGATGTTTTCTCCTACGGAACAAGAATTAT | |
| | TAAGGAAAATCTAACAACTGGTCATATAGAAGAAAATCCTGAATACGATCCGACA | |
| | GAAGAGCTTAAATCTCTGCTTTCCTCAAGAGGAATTGAGTATCAAAAAGGTCAAAA | |
| | TTTACTAGAAACAATACCTACTAGTGATATGACTAGAGAATTTTGGAATTCTCTTTT | |
| | CAAGATTTTTAAAGCAATTTTACAAATGAGAAACAGTCTAACTAATTCACCAATAGA | |
| | CAGGCTTTTATCTCCAGTTAAAGGAAAAGATGGAACCTTCTTTGATACAGATAAAG | |
| | TAGAAGGTACTAAGTTTGAAAAGTTAAAAGACGCTGATGCAAACGGAGCATATAA | |
| | CATTGCGTTAAAAGGATTGTTAGTCCTCGAGAAAAATGATTCTGTAGAGTCCAATA | |
| | AGGATCTAAAAAATGTTAAGAAAATTAGTCTTGAGGATTGGTTAAAGTTTGTCCAA | |
| | ATCACATTAAGAGATTAA | 35 |
| Codon | AGCAAATTGTCGACCTTCAATGAGCACTTTCAGAAAACCCTGACCCTGCGGAATG | |
| optimized | AGCTGGTGCCCGTGGGCAAGACACTGGAGAACATCATCAGCTCTAACGTGCTGA | |
| coding | TCAACGACGAGAAGCGGTCCGAGGACTACAAAAAGGCCAAGGAAATCATTGACA | |
| sequence (no | GCTATCACCGGGAGTTCATCGAGAAAAGCCTGAGCTCTGTGAATGTGGACTGGA | |
| N-terminal | ATGATCTGTACAGCTACCTGAGCAAGAAAGAACCCGAGGACTATGCCCAGAAAC | |
| methionine, no | AGAAGTTCCTGGAGGAGTTAGAGAACATCCTGCTGGAAAAGAGAAAGATCATCGT | |
| stop codon) | GAAGCAGTTCGAGCAGTACGTGTTCGGTTCCTATACCGACAGCAAGGGAAAAAA | |
| | GACCAAGGACCTGAAATTCGAAAACCTGTTTAAGTCCGAACTCTTTGACTACCTG | |
| | CTGCCTAACTTCTTGAAAAACGACGAGGATAAGAAGGTGATTGGCTCCTTCAATA | |
| | AGTTCACCAGCTATTTCACCGGCTTTTACGAGAACAGAAAAAACCTGTACAAGAG | |
| | CGAGCCTCTGCCTACCGCCGTCGCCTACAGAATCGTGAACGAGAACTTCCCCAA | |
| | GTTTATCTCTAACAAGAACATCTTTAGAGTGTGGAAGGACAACGTCCCTCAATTCA | |
| | TCGAGATCGCAAAGACCAAACTGAGAGAAGAAGGCATCTCTGATCTGAACATCGA | |
| | GCTGAAGTTTGATTTGACAAATTTCAACTCCTGCCTGAATCAGACCGGCATCGAT | |
| | ACCTACAACGACCTGATCGGCCAGCTGAACTTTGCTATCAACCTCGAATGTCAGA | |
| | AGGACAAGAACCTTTGTGACCTGCTGCGCAAGAAGCGGAGCCTTAAGATGGTGC | |
| | CACTGTACAAGCAAATCCTGTCCGACAACGATAGCAGCTTCAGCATCGACGAGTT | |
| | CGACAATGATGAAAGCGCCATCAAGGACGTTATCAGCTTCTACAAGAAGATGATC | |
| | GGCGAGAACTGCCCTCAGCGGACCCTGTCTGAGCTGCTGCACGGCCTGTCTAG | |
| | CCACGATCTGGAGAAAATTTTCGTGCAAGGGAAGAACCTGAACAGCGTGTCCAA | |
| | GAACCTGTTCGGCGGCAAGAACTGGTCCCTGCTGCGGGACGCCGTGATCGAGG | |
| | AAAAAAGCAAAGAGAAGGTGTTCAAGAAGGTGATCAAGAGCAACAGCACCGCTG | |
| | ATGAGCTGGATAAGGTGCTGTCTAAGGAGGAGTTCAGCATCTCTTTCCTATCCAA | |
| | GGTGTCCGGCAAGGATCTGAGCGTGGAAATCGACAAGTTCGTCAAAAAACAGGA | |
| | CGAGCTTCTGGTGGAGAACAATATCCAGAACTGGCCTTCTTCTCTCAAGAATAGC | |
| | GAAGAAAAGAACCTGATCAAGGCCCCTCTGGACTTTTTGTTGAATTTCTACAGGT | |
| | TCGCCCAGAGCTTCAGCAGCAACAACATCGATAAAGATATGTCCTTCTACGCTGA | |
| | TTTTGACGAGTCTCTGTCAAGCCTGGAAAATGTGATAGGCCTGTACAACAAAGTG | |
| | CGGAACTACGCCACCAAGAAACCTTACACACTGGAAAAGATCAAGCTAAACTTCG | |
| | AGAACCCTAACCTGGCCTCTGGATGGAGTGAGAGCAAGGAAAACGATTGCCTGA | |
| | GTATCATCCTGCTGAAGGAGAAGAAATACTTCCTGGGCATCTTCAACAAGAACAA | |
| | CAAGCCCAACTTTTCAGAGGGCATCAGCCACAGCCTGTCAAGCAACGGCTGTTA | |
| | CCGGAAGATGAGATACCTGCTGTTCAAGGGATTCAACAAGATGCTGCCTAAGTG | |
| | CGCCTTCACAGGAGAGGTGAAGGACCACTTCAAGGAAAGCTCCGATGACTTCAG | |
| | CCTGTTCAACAAGGACACCTTCATCAGCCCCCTGGTGATCACCAAGGAAATTTTC | |
| | GATCTGGCTTGCAGCAAGGAAAAAGTGAAGAAGTACCAAAAAGAATACGAGAAAA | |
| | TCAACAGAGCCGAGTACCGGCAGTCTCTGGTGAAGTGGATCACCTTTGGCCTGA | |
| | AGTTTCTGTCTAGCTACAAAACCACCACCCAGTTCGACCTGAGCAATTTGAAGCG | |
| | CCCCGAGGAATACTGCGACCTGAAAGAATTTTACGAGGACGTGGATAACTTAACC | |
| | TACAAGATTGAGTTCCTGAACATTAAAGAGGAGGACGTGGACGCTCTGGTCGAG | |
| | AAAGGCCAGCTGTACCTGTTTGAGATTAGAAACAAGGACTTCGCCAAGAATGCCA | |
| | GCGGCACGCCCAACCTGCATACACTGTATTTCAAGAGCATCTTCGATAGCAAGAA | |
| | CCTGGAAAATGGCATCGTGAAACTGAACGGCGAGGCCGAAATTTTCTACAGAAA | |
| | GAAGAGCCTGAAGAAGGATGATATCACCGTGCACAGAGAGGGAAGCTACCTCGT | |
| | CAACAAAGTCTGCGTGGACCCTAATTCCGGCAAGACAGAGCAGATCCCAGATAA | |
| | GATCTACGAGAACATCTACGCCTTCGTCAACGGCAAGTCACGGGACCTGAGCAA | |
| | GGAGGACGAGGTGTACTACGCCAAAGCCACCATCAAGAAGGCTACCCACGAGAT | |
| | CGTGAAGGATCGAAGATTCACCGTCGACAAGTTCTTCTTCCACTGCCCCATCACT | |
| | ATCAACTACAAGAGCAAAGACAAGCCAAGCAAGTTTAACGACAAAGTGCTGGACT | |
| | TCCTGAGAAATAACAAGGACATCAATATCATCGGCATCGACAGAGGCGAAAGAAA | |
| | CTTGATCTACGTGACCGTGATCAACCAGAACGGAGAGATCATCGACTGTAAGAG | |
| | CTTCAATACCATTAAGCACCAGAGCAGCACAGTGAACTACGACGTGGACTACCAC | |
| | AACAAGCTGCAGGAGCGGGAAAAGAACAGAAAGGAAGAAAAGAGATCTTGGAAC | |
| | AGCATCACCAAGATCGCCGATCTGAAAGAGGGCTACCTGTCTGCCGTGATTCAC | |
| | GAGGTTAGCCTGATGATGGTGAAGTACAACGCCATAGTTGTGATGGAAAACCTGA | |
| | ACCAGGGCTTCAAGAGAATCCGGGGCGGCATCGCCGAACGGAGCGTGTACCAA | |
| | AAGTTTGAAAAGATGCTCATCGACAAGCTGAACTACTTCGTGATCAAGAACGAGA | |
| | ACTGGACCAATCCTGGCGGAGTGCTGAATGGATACCAGCTGACAAACAAGGTGT | |
| | CCACAATCAAGGATATTGGAAATCAGTGCGGCTTCCTGTTCTACGTGCCCGCCAC | |
| | TTATACATCTAAAATCGATCCTAGCACTGGATTTGTGAACCTGATCAACTTCAACA | |
| | AGTACAAGAACAGCGAGGACAGAAGGAAGCTGATCTGTAGCTTCGACAAGATCT | |
| | GCTTTGTGCAGAATGAGAACCTGTTCAAGTTCTCTATCGATTACGGCAAACTGTG | |
| | CCCTGACAGCAAGATCGCCATCAAAAAGTGGGACGTATTCTCCTATGGCACCAG | |
| | GATCATCAAGGAAAACCTGACAACAGGCCACATCGAAGAAAATCCAGAGTACGA | |
| | CCCTACAGAGGAACTGAAATCCCTGCTTTCCAGCAGAGGCATCGAGTACCAGAA | |
| | GGGCCAAAACCTGCTAGAAACCATCCCTACCAGCGACATGACCAGAGAGTTCTG | |
| | GAATAGCCTGTTCAAGATCTTCAAGGCCATCCTGCAGATGAGAAACTCTCTGACA | |
| | AACTCTCCTATCGACCGGCTGCTAAGCCCTGTGAAGGGGAAAGATGGAACCTTC | |
| | TTCGACACCGACAAGGTGGAAGGCACAAAATTTGAGAAACTGAAGGACGCTGAC | |
| | GCTAACGGCGCCTACAACATCGCCCTGAAGGGCCTGCTGGTGCTGGAAAAAAAC | |
| | GACTCTGTCGAGAGCAACAAGGACCTCAAGAACGTGAAGAAAATCTCACTGGAG | |
| | GACTGGCTGAAATTCGTGCAGATCACACTTAGAGAC | |
| Expression | ATGggcAGCAAATTGTCGACCTTCAATGAGCACTTTCAGAAAACCCTGACCCTGCG | 36 |
| construct (with | GAATGAGCTGGTGCCCGTGGGCAAGACACTGGAGAACATCATCAGCTCTAACGT | |
| N-terminal | GCTGATCAACGACGAGAAGCGGTCCGAGGACTACAAAAAGGCCAAGGAAATCAT | |
| methionine | TGACAGCTATCACCGGGAGTTCATCGAGAAAAGCCTGAGCTCTGTGAATGTGGA | |
| and stop | CTGGAATGATCTGTACAGCTACCTGAGCAAGAAAGAACCCGAGGACTATGCCCA | |
| codon, | GAAACAGAAGTTCCTGGAGGAGTTAGAGAACATCCTGCTGGAAAAGAGAAAGAT | |
| includes V5- | CATCGTGAAGCAGTTCGAGCAGTACGTGTTCGGTTCCTATACCGACAGCAAGGG | |
| tag and C- | AAAAAAGACCAAGGACCTGAAATTCGAAAACCTGTTTAAGTCCGAACTCTTTGACT | |
| terminal NLS) | ACCTGCTGCCTAACTTCTTGAAAAACGACGAGGATAAGAAGGTGATTGGCTCCTT | |
| | CAATAAGTTCACCAGCTATTTCACCGGCTTTTACGAGAACAGAAAAAACCTGTACA | |
| | AGAGCGAGCCTCTGCCTACCGCCGTCGCCTACAGAATCGTGAACGAGAACTTCC | |
| | CCAAGTTTATCTCTAACAAGAACATCTTTAGAGTGTGGAAGGACAACGTCCCTCA | |
| | ATTCATCGAGATCGCAAAGACCAAACTGAGAGAAGAAGGCATCTCTGATCTGAAC | |
| | ATCGAGCTGAAGTTTGATTTGACAAATTTCAACTCCTGCCTGAATCAGACCGGCA | |
| | TCGATACCTACAACGACCTGATCGGCCAGCTGAACTTTGCTATCAACCTCGAATG | |
| | TCAGAAGGACAAGAACCTTTGTGACCTGCTGCGCAAGAAGCGGAGCCTTAAGAT | |
| | GGTGCCACTGTACAAGCAAATCCTGTCCGACAACGATAGCAGCTTCAGCATCGA | |
| | CGAGTTCGACAATGATGAAAGCGCCATCAAGGACGTTATCAGCTTCTACAAGAAG | |
| | ATGATCGGCGAGAACTGCCCTCAGCGGACCCTGTCTGAGCTGCTGCACGGCCT | |
| | GTCTAGCCACGATCTGGAGAAAATTTTCGTGCAAGGGAAGAACCTGAACAGCGT | |
| | GTCCAAGAACCTGTTCGGCGGCAAGAACTGGTCCCTGCTGCGGGACGCCGTGA | |
| | TCGAGGAAAAAAGCAAAGAGAAGGTGTTCAAGAAGGTGATCAAGAGCAACAGCA | |
| | CCGCTGATGAGCTGGATAAGGTGCTGTCTAAGGAGGAGTTCAGCATCTCTTTCCT | |
| | ATCCAAGGTGTCCGGCAAGGATCTGAGCGTGGAAATCGACAAGTTCGTCAAAAA | |
| | ACAGGACGAGCTTCTGGTGGAGAACAATATCCAGAACTGGCCTTCTTCTCTCAAG | |
| | AATAGCGAAGAAAAGAACCTGATCAAGGCCCCTCTGGACTTTTTGTTGAATTTCTA | |
| | CAGGTTCGCCCAGAGCTTCAGCAGCAACAACATCGATAAAGATATGTCCTTCTAC | |
| | GCTGATTTTGACGAGTCTCTGTCAAGCCTGGAAAATGTGATAGGCCTGTACAACA | |
| | AAGTGCGGAACTACGCCACCAAGAAACCTTACACACTGGAAAAGATCAAGCTAAA | |
| | CTTCGAGAACCCTAACCTGGCCTCTGGATGGAGTGAGAGCAAGGAAAACGATTG | |
| | CCTGAGTATCATCCTGCTGAAGGAGAAGAAATACTTCCTGGGCATCTTCAACAAG | |
| | AACAACAAGCCCAACTTTTCAGAGGGCATCAGCCACAGCCTGTCAAGCAACGGC | |
| | TGTTACCGGAAGATGAGATACCTGCTGTTCAAGGGATTCAACAAGATGCTGCCTA | |
| | AGTGCGCCTTCACAGGAGAGGTGAAGGACCACTTCAAGGAAAGCTCCGATGACT | |
| | TCAGCCTGTTCAACAAGGACACCTTCATCAGCCCCCTGGTGATCACCAAGGAAAT | |
| | TTTCGATCTGGCTTGCAGCAAGGAAAAAGTGAAGAAGTACCAAAAAGAATACGAG | |
| | AAAATCAACAGAGCCGAGTACCGGCAGTCTCTGGTGAAGTGGATCACCTTTGGC | |
| | CTGAAGTTTCTGTCTAGCTACAAAACCACCACCCAGTTCGACCTGAGCAATTTGA | |
| | AGCGCCCCGAGGAATACTGCGACCTGAAAGAATTTTACGAGGACGTGGATAACT | |
| | TAACCTACAAGATTGAGTTCCTGAACATTAAAGAGGAGGACGTGGACGCTCTGGT | |
| | CGAGAAAGGCCAGCTGTACCTGTTTGAGATTAGAAACAAGGACTTCGCCAAGAAT | |
| | GCCAGCGGCACGCCCAACCTGCATACACTGTATTTCAAGAGCATCTTCGATAGCA | |
| | AGAACCTGGAAAATGGCATCGTGAAACTGAACGGCGAGGCCGAAATTTTCTACA | |
| | GAAAGAAGAGCCTGAAGAAGGATGATATCACCGTGCACAGAGAGGGAAGCTACC | |
| | TCGTCAACAAAGTCTGCGTGGACCCTAATTCCGGCAAGACAGAGCAGATCCCAG | |
| | ATAAGATCTACGAGAACATCTACGCCTTCGTCAACGGCAAGTCACGGGACCTGA | |
| | GCAAGGAGGACGAGGTGTACTACGCCAAAGCCACCATCAAGAAGGCTACCCACG | |
| | AGATCGTGAAGGATCGAAGATTCACCGTCGACAAGTTCTTCTTCCACTGCCCCAT | |
| | CACTATCAACTACAAGAGCAAAGACAAGCCAAGCAAGTTTAACGACAAAGTGCTG | |
| | GACTTCCTGAGAAATAACAAGGACATCAATATCATCGGCATCGACAGAGGCGAAA | |
| | GAAACTTGATCTACGTGACCGTGATCAACCAGAACGGAGAGATCATCGACTGTAA | |
| | GAGCTTCAATACCATTAAGCACCAGAGCAGCACAGTGAACTACGACGTGGACTA | |
| | CCACAACAAGCTGCAGGAGCGGGAAAAGAACAGAAAGGAAGAAAAGAGATCTTG | |
| | GAACAGCATCACCAAGATCGCCGATCTGAAAGAGGGCTACCTGTCTGCCGTGAT | |
| | TCACGAGGTTAGCCTGATGATGGTGAAGTACAACGCCATAGTTGTGATGGAAAAC | |
| | CTGAACCAGGGCTTCAAGAGAATCCGGGGCGGCATCGCCGAACGGAGCGTGTA | |
| | CCAAAAGTTTGAAAAGATGCTCATCGACAAGCTGAACTACTTCGTGATCAAGAAC | |
| | GAGAACTGGACCAATCCTGGCGGAGTGCTGAATGGATACCAGCTGACAAACAAG | |
| | GTGTCCACAATCAAGGATATTGGAAATCAGTGCGGCTTCCTGTTCTACGTGCCCG | |
| | CCACTTATACATCTAAAATCGATCCTAGCACTGGATTTGTGAACCTGATCAACTTC | |
| | AACAAGTACAAGAACAGCGAGGACAGAAGGAAGCTGATCTGTAGCTTCGACAAG | |
| | ATCTGCTTTGTGCAGAATGAGAACCTGTTCAAGTTCTCTATCGATTACGGCAAACT | |
| | GTGCCCTGACAGCAAGATCGCCATCAAAAAGTGGGACGTATTCTCCTATGGCAC | |
| | CAGGATCATCAAGGAAAACCTGACAACAGGCCACATCGAAGAAAATCCAGAGTA | |
| | CGACCCTACAGAGGAACTGAAATCCCTGCTTTCCAGCAGAGGCATCGAGTACCA | |
| | GAAGGGCCAAAACCTGCTAGAAACCATCCCTACCAGCGACATGACCAGAGAGTT | |
| | CTGGAATAGCCTGTTCAAGATCTTCAAGGCCATCCTGCAGATGAGAAACTCTCTG | |
| | ACAAACTCTCCTATCGACCGGCTGCTAAGCCCTGTGAAGGGGAAAGATGGAACC | |
| | TTCTTCGACACCGACAAGGTGGAAGGCACAAAATTTGAGAAACTGAAGGACGCT | |
| | GACGCTAACGGCGCCTACAACATCGCCCTGAAGGGCCTGCTGGTGCTGGAAAAA | |
| | AACGACTCTGTCGAGAGCAACAAGGACCTCAAGAACGTGAAGAAAATCTCACTG | |
| | GAGGACTGGCTGAAATTCGTGCAGATCACACTTAGAGACtctagaAAGCGGACAGC | |
| | AGACGGCTCCGAATTTGAAAGCCCTAAGAAAAAGAGAAAGGTGggatccGGCAAAC | |
| | CTATCCCCAATCCCCTGCTGGGCCTGGACAGCACCTGA | |

In some embodiments a ZZGY Type V Cas protein comprises an amino acid sequence of SEQ ID NO: 31, SEQ ID NO:32, or SEQ ID NO:33. In some embodiments, a ZZGY Type V Cas protein has nickase activity, for example resulting from one or more amino acid substitutions relative to the sequence of SEQ ID NO:31, SEQ ID NO:32, or SEQ ID NO:33. In some embodiments, the one or more amino acid substitutions providing nickase activity comprise a D905 substitution, wherein the position of the D905 substitution is defined with respect to the amino acid numbering of SEQ ID NO:32 (corresponding to amino acid 908 of SEQ ID NO: 121). In some embodiments, the one or more amino acid substitutions providing nickase activity comprise an E998 substitution, wherein the position of the E998 substitution is defined with respect to the amino acid numbering of SEQ ID NO:32 (corresponding to amino acid 993 of SEQ ID NO: 121). In some embodiments, the one or more amino acid substitutions providing nickase activity comprise a R1214 substitution, wherein the position of the R1214 substitution is defined with respect to the amino acid numbering of SEQ ID NO:32 (corresponding to amino acid 1226 of SEQ ID NO: 121). In some embodiments, the one or more amino acid substitutions providing nickase activity comprise a D1254 substitution, wherein the position of the D1254 substitution is defined with respect to the amino acid numbering of SEQ ID NO:32 (corresponding to amino acid 1263 of SEQ ID NO: 121). In some embodiments, a ZZGY Type V Cas protein is catalytically inactive, for example due to a R1214 substitution in combination with a D905 substitution, a E998 substitution, and/or D1254 substitution.

### 6.2.7. ZKBG Type V Cas Proteins

In one aspect, the disclosure provides ZKBG Type V Cas proteins. ZKBG Type V Cas proteins can be further classified as Type V-A Cas proteins. The ZKBG Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:37. In some embodiments, the ZKBG Type V Cas proteins comprise an amino acid sequence that is at least 95%, at least 96%, at least 97%, at least 98%, or at least 99% identical to SEQ ID NO:37. In some embodiments, a ZKBG Type V Cas protein comprises an amino acid sequence that is identical to SEQ ID NO:37.

Exemplary ZKBG Type V Cas protein sequences and nucleotide sequences encoding exemplary ZKBG Type V Cas proteins are set forth in Table 1G.

TABLE 1G
ZKBG Type V Cas Sequences

| | | SEQ |
|---|---|---|
| | | ID |
| Name | Sequence | NO. |
| Wildtype | KRLIDFTNIYQRSKTLRFRLEPIGKTADYIKNSQSLETDARLAKESKKVKELADEYHKE | 37 |
| amino acid | FIGDVLSSLELPLSKINELWDIYIYIYMSNDTDREIKFKKLQENLRKVIAEAFSKDKRFG | |
| sequence | NLFKKEIITDILPEFLQDKDDDIKIVNRFKGFTTYFYAFHKNRENMYVSEEKSTAIPYRIV | |
| (without N- | NQNLVKYFDNYKTFKEKVMPLLKDKNIVESIERDFKDILNEKSIEDVFGLANFTHTLCQ | |
| terminal | ADIEKYNTLIGGLVVKNEKKEIKGINQYINEHNQTSKKGNGIPKLKPLFNQILSDRKSLS | |
| methionine) | FTLDDIKKTSEAIRTIKDEYENLRDKLATIERLIKSIKEYDLAGIYIKMGEDTSTISQHWF | |
| | GAYYKIIEAIADAWERRNPKKNRESKAYSKYVSSLKSISLQEIDDLKIGEPIENYFATFG | |
| | TTCSDRTSGVSSLNRIKAAYTEFVNKFPEGFEDGDDCNDAYFKANVEVVKNLLDSIK | |
| | DFQRFVKPLLGNEDERDKDEAFYGEFVPTYTDMDNIITPLYNRVRNFATKKPYSTDKI | |
| | KINFENVVLLKGWDKNKESDYASIILMKDGQYFLGVLRNGSKSTLKTILPNTGDCYQK | |
| | MVYKYFKDIKSNLPRCTTQRKDVKAHFAESSDDYTLLDTKAFVSALTISREVFELYNA | |
| | PDKEKKFKKEYLKNTNDSIGYANAVSVCKRFCLEFLKKYRSTAIYDLSDVETSVDSFD | |
| | DLSSFYQEIDKRLYSISFENVSVDSVNELVDNGNMLLFRIANKDFSPNSKGRPNLHTIY | |
| | WRMLFDPANLKDVVYQLNGNAEIFFRKASVTRTEPTHPANVAIKNKSEYNKQNKPYS | |
| | TFKYGLIKDRRYTTDQFEFHVPITMNFKQPESSKLQDKLNKQVLDFLKQDGVRHIIGID | |
| | RGERNLLYLVMVDMEGKIKKQISLNEIAGNPKNPEFKQDFLALLHEREGDRLESRRS | |
| | WNTIQSIKELKEGYMSLVVHEIANMMLENDAIVVLENLNRSFMQKRGGIEKSVYQKFE | |
| | KMLIDKLGYIVDKTKDVSDNGGALHAVQLADTFENFNKTQKGAIRQCGFIFYIPAWRT | |
| | SKIDPVTGFVPMLRCQYESIVESKKFFGKFDSIYYDATGKYFVFQTDFTKFNTESKGGI | |
| | QKWDICTYGDRIYAPRTKDRNNNPVSERVNLTEEMKSLFVSHNINIQGDIKAGIMQQT | |
| | DKEFFESLHRLLRLTLQIRNSKKSTGKDYEDYIISPVMGKDGRFFDSRNADATQPKDA | |
| | DANGAYNIARKGLMLLRQIQAQEKQDLSNGKWLEFAQR | |
| Wildtype | MKRLIDFTNIYQRSKTLRFRLEPIGKTADYIKNSQSLETDARLAKESKKVKELADEYHK | 38 |
| amino acid | EFIGDVLSSLELPLSKINELWDIYIYIYMSNDTDREIKFKKLQENLRKVIAEAFSKDKRFG | |
| sequence (with | NLFKKEIITDILPEFLQDKDDDIKIVNRFKGFTTYFYAFHKNRENMYVSEEKSTAIPYRIV | |
| N-terminal | NQNLVKYFDNYKTFKEKVMPLLKDKNIVESIERDFKDILNEKSIEDVFGLANFTHTLCQ | |
| methionine) | ADIEKYNTLIGGLVVKNEKKEIKGINQYINEHNQTSKKGNGIPKLKPLFNQILSDRKSLS | |
| | FTLDDIKKTSEAIRTIKDEYENLRDKLATIERLIKSIKEYDLAGIYIKMGEDTSTISQHWF | |
| | GAYYKIIEAIADAWERRNPKKNRESKAYSKYVSSLKSISLQEIDDLKIGEPIENYFATFG | |
| | TTCSDRTSGVSSLNRIKAAYTEFVNKFPEGFEDGDDCNDAYFKANVEVVKNLLDSIK | |
| | DFQRFVKPLLGNEDERDKDEAFYGEFVPTYTDMDNIITPLYNRVRNFATKKPYSTDKI | |
| | KINFENVVLLKGWDKNKESDYASIILMKDGQYFLGVLRNGSKSTLKTILPNTGDCYQK | |
| | MVYKYFKDIKSNLPRCTTQRKDVKAHFAESSDDYTLLDTKAFVSALTISREVFELYNA | |
| | PDKEKKFKKEYLKNTNDSIGYANAVSVCKRFCLEFLKKYRSTAIYDLSDVETSVDSFD | |
| | DLSSFYQEIDKRLYSISFENVSVDSVNELVDNGNMLLFRIANKDFSPNSKGRPNLHTIY | |
| | WRMLFDPANLKDVVYQLNGNAEIFFRKASVTRTEPTHPANVAIKNKSEYNKQNKPYS | |
| | TFKYGLIKDRRYTTDQFEFHVPITMNFKQPESSKLQDKLNKQVLDFLKQDGVRHIIGID | |
| | RGERNLLYLVMVDMEGKIKKQISLNEIAGNPKNPEFKQDFLALLHEREGDRLESRRS | |
| | WNTIQSIKELKEGYMSLVVHEIANMMLENDAIVVLENLNRSFMQKRGGIEKSVYQKFE | |
| | KMLIDKLGYIVDKTKDVSDNGGALHAVQLADTFENFNKTQKGAIRQCGFIFYIPAWRT | |
| | SKIDPVTGFVPMLRCQYESIVESKKFFGKFDSIYYDATGKYFVFQTDFTKFNTESKGGI | |
| | QKWDICTYGDRIYAPRTKDRNNNPVSERVNLTEEMKSLFVSHNINIQGDIKAGIMQQT | |
| | DKEFFESLHRLLRLTLQIRNSKKSTGKDYEDYIISPVMGKDGRFFDSRNADATQPKDA | |
| | DANGAYNIARKGLMLLRQIQAQEKQDLSNGKWLEFAQR | |
| Expression | MGKRLIDFTNIYQRSKTLRFRLEPIGKTADYIKNSQSLETDARLAKESKKVKELADEYH | 39 |
| construct (with | KEFIGDVLSSLELPLSKINELWDIYIYIYMSNDTDREIKFKKLQENLRKVIAEAFSKDKRF | |
| N-terminal | GNLFKKEIITDILPEFLQDKDDDIKIVNRFKGFTTYFYAFHKNRENMYVSEEKSTAIPYRI | |
| methionine, | VNQNLVKYFDNYKTFKEKVMPLLKDKNIVESIERDFKDILNEKSIEDVFGLANFTHTLC | |
| V5-tag and C- | QADIEKYNTLIGGLVVKNEKKEIKGINQYINEHNQTSKKGNGIPKLKPLFNQILSDRKSL | |
| terminal NLS) | SFTLDDIKKTSEAIRTIKDEYENLRDKLATIERLIKSIKEYDLAGIYIKMGEDTSTISQHWF | |
| aa sequence | GAYYKIIEAIADAWERRNPKKNRESKAYSKYVSSLKSISLQEIDDLKIGEPIENYFATFG | |
| | TTCSDRTSGVSSLNRIKAAYTEFVNKFPEGFEDGDDCNDAYFKANVEVVKNLLDSIK | |
| | DFQRFVKPLLGNEDERDKDEAFYGEFVPTYTDMDNIITPLYNRVRNFATKKPYSTDKI | |
| | KINFENVVLLKGWDKNKESDYASIILMKDGQYFLGVLRNGSKSTLKTILPNTGDCYQK | |
| | MVYKYFKDIKSNLPRCTTQRKDVKAHFAESSDDYTLLDTKAFVSALTISREVFELYNA | |
| | PDKEKKFKKEYLKNTNDSIGYANAVSVCKRFCLEFLKKYRSTAIYDLSDVETSVDSFD | |
| | DLSSFYQEIDKRLYSISFENVSVDSVNELVDNGNMLLFRIANKDFSPNSKGRPNLHTIY | |
| | WRMLFDPANLKDVVYQLNGNAEIFFRKASVTRTEPTHPANVAIKNKSEYNKQNKPYS | |
| | TFKYGLIKDRRYTTDQFEFHVPITMNFKQPESSKLQDKLNKQVLDFLKQDGVRHIIGID | |
| | RGERNLLYLVMVDMEGKIKKQISLNEIAGNPKNPEFKQDFLALLHEREGDRLESRRS | |
| | WNTIQSIKELKEGYMSLVVHEIANMMLENDAIVVLENLNRSFMQKRGGIEKSVYQKFE | |
| | KMLIDKLGYIVDKTKDVSDNGGALHAVQLADTFENFNKTQKGAIRQCGFIFYIPAWRT | |
| | SKIDPVTGFVPMLRCQYESIVESKKFFGKFDSIYYDATGKYFVFQTDFTKFNTESKGGI | |
| | QKWDICTYGDRIYAPRTKDRNNNPVSERVNLTEEMKSLFVSHNINIQGDIKAGIMQQT | |
| | DKEFFESLHRLLRLTLQIRNSKKSTGKDYEDYIISPVMGKDGRFFDSRNADATQPKDA | |
| | DANGAYNIARKGLMLLRQIQAQEKQDLSNGKWLEFAQRSRKRTADGSEFESPKKKR | |
| | KVGSGKPIPNPLLGLDST | |
| Wildtype | ATGAAACGCCTAATTGACTTTACAAACATCTATCAGCGATCAAAGACTTTGAGGTT | 40 |
| coding | TCGATTGGAGCCTATCGGTAAAACGGCCGACTATATTAAGAATTCTCAGTCCCTC | |
| sequence (with | GAAACTGATGCGCGTTTGGCAAAAGAGAGCAAGAAGGTAAAAGAGCTTGCTGAT | |
| N-terminal | GAATATCACAAAGAGTTTATTGGAGATGTCCTGTCTTCGTTGGAATTGCCTTTAAG | |
| methionine | CAAAATCAACGAGTTATGGGATATATATATATATATATATATGTCCAATGATACAGA | |
| and stop | CCGCGAGATAAAATTCAAAAAACTGCAAGAGAACCTGCGAAAGGTGATTGCAGA | |
| codon) | GGCTTTTAGTAAGGACAAACGGTTTGGTAATTTATTCAAAAAGGAGATAATCACAG | |
| | ACATTCTGCCGGAATTCTTGCAAGATAAGGATGATGATATTAAGATCGTAAATAGA | |
| | TTCAAAGGATTTACCACATATTTTTACGCCTTTCATAAGAATAGGGAAAATATGTAT | |
| | GTCTCGGAAGAGAAATCGACTGCAATACCATATCGAATTGTGAATCAAAATCTCG | |
| | TCAAGTATTTTGACAACTACAAGACGTTCAAAGAGAAGGTAATGCCTCTTCTGAAA | |
| | GACAAGAATATAGTCGAAAGCATAGAGAGAGACTTCAAAGACATCTTGAACGAAA | |
| | AATCAATAGAGGATGTTTTTGGCCTTGCCAACTTCACTCATACTTTATGTCAGGCT | |
| | GACATCGAGAAATACAATACGTTGATAGGTGGCCTTGTCGTCAAAAACGAAAAAA | |
| | AAGAGATTAAAGGTATTAATCAGTACATTAACGAACATAACCAAACGAGTAAAAAA | |
| | GGGAATGGAATTCCGAAACTAAAGCCGTTGTTCAATCAGATTTTGAGCGATAGAA | |
| | AATCGTTATCGTTTACCTTAGACGATATCAAAAAAACGTCGGAGGCTATTCGCAC | |
| | CATTAAGGATGAGTATGAAAATCTCCGAGACAAGTTGGCGACCATCGAAAGGCTT | |
| | ATTAAGTCTATCAAGGAGTATGATCTTGCAGGTATTTACATCAAGATGGGAGAGG | |
| | ATACTTCGACAATATCGCAGCATTGGTTTGGTGCGTATTATAAAATCATCGAAGCG | |
| | ATAGCAGATGCATGGGAACGACGAAATCCGAAGAAAAACAGAGAATCCAAGGCA | |
| | TATAGCAAGTATGTATCGTCCCTAAAAAGCATCAGTCTCCAAGAAATAGATGATCT | |
| | CAAAATCGGAGAGCCTATAGAGAACTACTTCGCAACTTTTGGCACGACTTGTTCA | |
| | GACCGAACAAGTGGAGTTTCTTCGCTCAATAGGATAAAAGCTGCTTATACCGAGT | |
| | TCGTGAACAAATTTCCTGAAGGATTTGAAGATGGCGATGACTGTAACGATGCCTA | |
| | CTTTAAGGCTAATGTGGAAGTCGTCAAAAATCTGCTGGATTCAATTAAAGATTTTC | |
| | AGCGTTTTGTGAAGCCTTTGCTTGGCAATGAGGACGAAAGAGACAAAGACGAGG | |
| | CATTCTATGGAGAGTTTGTCCCGACATACACAGATATGGATAACATCATAACCCCT | |
| | CTATACAACCGTGTACGCAATTTTGCCACCAAGAAACCATACTCTACAGACAAGA | |
| | TAAAAATCAACTTTGAAAACGTAGTATTGCTAAAAGGATGGGACAAAAACAAGGA | |
| | GTCAGACTACGCATCCATCATATTGATGAAAGACGGACAATACTTTTTAGGGGTA | |
| | CTCCGTAATGGTTCAAAAAGTACTCTTAAAACCATATTGCCTAACACAGGTGATTG | |
| | CTATCAAAAAATGGTTTATAAGTATTTTAAGGATATAAAATCAAATCTTCCCCGGTG | |
| | TACGACCCAGAGGAAAGACGTGAAAGCGCACTTTGCCGAATCGAGCGACGATTA | |
| | CACTCTTTTAGATACAAAGGCCTTTGTTTCGGCACTGACTATCAGCAGAGAAGTG | |
| | TTCGAACTATACAATGCCCCCGATAAGGAGAAAAAATTCAAAAAGGAATATTTGAA | |
| | GAACACAAACGATAGTATAGGCTACGCCAATGCTGTATCCGTATGTAAACGCTTC | |
| | TGTTTGGAGTTCCTAAAAAAATATCGCAGCACTGCCATATATGATCTTTCGGATGT | |
| | TGAAACTTCAGTCGATTCGTTTGACGATTTGTCCTCATTCTATCAAGAGATAGACA | |
| | AAAGGCTGTACAGCATCTCATTCGAAAATGTATCTGTCGATTCCGTCAATGAGCTT | |
| | GTAGACAATGGCAATATGCTTCTATTCCGTATCGCGAATAAAGATTTTTCGCCTAA | |
| | CAGCAAGGGCCGTCCCAATCTTCATACTATATATTGGCGAATGCTTTTCGACCCG | |
| | GCCAACCTGAAGGATGTTGTATATCAGCTCAATGGTAATGCCGAAATATTCTTCC | |
| | GTAAGGCAAGCGTTACGAGGACGGAGCCTACACATCCGGCTAACGTTGCCATCA | |
| | AAAACAAGAGCGAATATAACAAACAGAATAAGCCGTATAGTACATTCAAGTACGG | |
| | TTTAATCAAGGATAGGCGCTACACTACCGACCAGTTCGAGTTTCATGTACCCATC | |
| | ACAATGAACTTCAAGCAACCAGAGTCGTCTAAACTACAGGACAAGCTCAACAAGC | |
| | AAGTGCTTGACTTCTTGAAACAGGACGGCGTACGCCATATTATAGGCATTGATCG | |
| | GGGCGAACGTAATCTGCTATACTTGGTGATGGTAGATATGGAGGGCAAAATCAAA | |
| | AAACAAATATCACTCAACGAGATAGCCGGTAATCCGAAGAATCCCGAGTTCAAAC | |
| | AAGACTTCCTTGCACTACTGCACGAGCGCGAAGGTGACCGTTTGGAGTCACGTC | |
| | GCAGTTGGAACACCATTCAGAGCATTAAAGAACTCAAAGAAGGTTACATGAGCTT | |
| | GGTGGTTCATGAAATAGCGAATATGATGCTTGAGAATGATGCTATAGTAGTGCTC | |
| | GAAAATCTGAATCGCTCGTTTATGCAAAAGCGCGGCGGCATAGAAAAGTCTGTAT | |
| | ACCAAAAGTTCGAAAAGATGCTTATCGACAAGTTGGGATACATCGTGGATAAGAC | |
| | TAAAGATGTGTCCGACAACGGAGGCGCACTACATGCTGTACAGCTTGCTGATAC | |
| | GTTTGAAAACTTCAATAAGACCCAAAAAGGAGCTATTCGTCAATGTGGATTCATAT | |
| | TCTATATTCCTGCATGGCGTACCAGCAAGATTGACCCCGTTACCGGCTTTGTGCC | |
| | AATGCTTAGGTGTCAATATGAAAGCATCGTAGAATCCAAAAAATTCTTCGGAAAGT | |
| | TCGACAGTATATACTACGATGCGACAGGAAAGTATTTTGTCTTCCAAACTGACTTT | |
| | ACCAAATTCAATACCGAGAGCAAAGGAGGAATCCAAAAATGGGATATATGCACCT | |
| | ATGGAGACAGAATATATGCTCCTCGCACCAAAGACCGGAATAATAACCCTGTTTC | |
| | GGAACGTGTAAACCTTACTGAGGAGATGAAATCACTGTTTGTATCGCATAATATCA | |
| | ATATTCAAGGCGATATCAAAGCCGGAATTATGCAGCAGACAGACAAGGAGTTCTT | |
| | CGAGTCACTGCATCGATTGCTTCGACTTACGTTGCAAATACGCAATAGCAAAAAA | |
| | TCTACAGGCAAAGACTATGAAGACTATATCATATCGCCGGTGATGGGCAAGGAC | |
| | GGTCGTTTCTTTGATTCGCGTAACGCGGATGCTACGCAACCTAAGGATGCAGATG | |
| | CCAATGGCGCGTACAATATTGCACGCAAAGGCTTGATGCTGCTTCGCCAGATTCA | |
| | AGCCCAAGAGAAGCAAGACCTATCCAACGGAAAATGGCTTGAATTTGCCCAAAG | |
| | GTGA | |
| Codon | AAGCGGCTCATCGACTTCACCAACATCTACCAGCGTTCTAAGACCCTGAGATTCA | 41 |
| optimized | GACTGGAACCTATCGGCAAGACCGCGGACTACATCAAAAACAGCCAGTCCCTGG | |
| coding | AAACAGACGCCAGACTGGCCAAGGAATCCAAGAAAGTGAAGGAACTGGCCGATG | |
| sequence (no | AGTACCACAAAGAGTTTATCGGCGACGTGCTGAGCAGCCTGGAGCTGCCCCTGA | |
| N-terminal | GCAAAATCAACGAGCTGTGGGACATCTATATCTACATCTACATGAGCAACGACAC | |
| methionine, no | CGATCGGGAAATCAAATTTAAGAAGCTCCAGGAGAACCTGCGGAAGGTGATCGC | |
| stop codon) | CGAGGCCTTTAGCAAGGATAAGAGATTCGGCAACCTGTTCAAGAAAGAAATCATC | |
| | ACAGATATCCTGCCCGAGTTCCTGCAAGATAAAGATGACGATATCAAAATCGTGA | |
| | ACCGGTTCAAGGGTTTTACAACCTACTTCTACGCCTTCCACAAGAATCGGGAAAA | |
| | CATGTACGTGTCTGAAGAGAAGAGCACAGCCATCCCCTACAGAATCGTGAATCAA | |
| | AACCTGGTGAAATACTTCGATAACTACAAGACTTTTAAGGAGAAGGTGATGCCTC | |
| | TGCTGAAGGACAAGAACATCGTCGAAAGCATCGAGCGCGACTTCAAGGACATCC | |
| | TGAACGAGAAAAGCATCGAGGACGTGTTCGGCCTGGCCAATTTCACCCACACCC | |
| | TGTGCCAGGCTGACATCGAGAAGTACAACACCTTGATAGGCGGACTGGTGGTGA | |
| | AGAACGAAAAGAAGGAGATCAAGGGCATCAACCAGTATATTAACGAGCACAACCA | |
| | GACCTCTAAGAAGGGCAACGGCATCCCAAAGCTGAAGCCTCTGTTTAACCAGAT | |
| | CCTGAGCGACAGAAAATCTCTCAGCTTCACCCTGGATGATATCAAGAAAACCAGC | |
| | GAGGCCATCAGAACAATTAAGGACGAGTATGAGAACCTGAGAGATAAGCTGGCC | |
| | ACAATCGAACGGCTGATCAAGAGCATCAAGGAATACGACCTGGCCGGCATCTAC | |
| | ATCAAGATGGGCGAGGACACCTCTACCATCTCCCAGCACTGGTTCGGTGCCTAT | |
| | TACAAGATTATCGAAGCCATCGCCGACGCCTGGGAGAGAAGAAACCCAAAGAAA | |
| | AACAGAGAGAGCAAGGCCTACAGCAAGTACGTGAGCAGCCTTAAGAGCATCAGC | |
| | CTGCAGGAGATCGACGACCTGAAGATCGGCGAGCCTATCGAGAATTACTTCGCC | |
| | ACCTTTGGAACAACATGTAGCGACCGGACATCTGGCGTGAGCTCTCTGAACCGG | |
| | ATCAAAGCCGCCTACACCGAGTTCGTGAACAAGTTCCCCGAGGGCTTTGAGGAT | |
| | GGCGATGATTGCAACGACGCTTACTTCAAAGCCAATGTGGAGGTGGTGAAGAAC | |
| | TTGCTGGATAGCATAAAAGACTTCCAGAGATTTGTGAAGCCTCTACTGGGCAATG | |
| | AGGACGAGCGGGACAAAGATGAGGCCTTCTACGGCGAGTTCGTTCCTACCTACA | |
| | CAGATATGGACAACATCATCACGCCTCTGTATAATAGAGTCAGAAACTTCGCTAC | |
| | CAAGAAGCCTTACAGTACAGACAAGATCAAAATAAACTTCGAAAACGTGGTACTG | |
| | CTGAAGGGCTGGGATAAGAACAAGGAGAGCGACTATGCCAGCATCATCCTGATG | |
| | AAGGACGGCCAGTACTTTCTGGGAGTGCTGAGAAACGGATCTAAGAGCACTCTG | |
| | AAAACCATCCTGCCTAACACCGGTGACTGCTACCAGAAAATGGTGTACAAGTATT | |
| | TCAAGGATATCAAGTCTAACCTGCCCAGATGCACCACCCAGAGAAAGGACGTGA | |
| | AGGCACATTTCGCTGAAAGCAGCGATGATTACACCCTGCTTGATACAAAAGCCTT | |
| | CGTGAGCGCTCTGACGATCTCCAGAGAGGTGTTCGAACTGTACAACGCTCCTGA | |
| | TAAGGAAAAGAAATTCAAGAAGGAATACCTGAAGAACACCAACGACTCCATCGGC | |
| | TACGCCAATGCAGTGAGCGTGTGCAAGAGATTCTGCCTGGAGTTCCTGAAAAAG | |
| | TACCGGAGCACCGCCATCTACGACCTGAGCGATGTTGAAACCTCTGTGGACAGT | |
| | TTCGACGACCTGAGCAGCTTCTACCAGGAGATCGATAAGAGACTGTACAGCATCA | |
| | GCTTCGAAAACGTGAGCGTGGACAGCGTGAACGAGCTGGTGGATAACGGCAATA | |
| | TGCTGCTGTTCAGAATCGCCAACAAGGATTTCTCTCCTAATAGCAAGGGCAGACC | |
| | TAATCTGCACACAATTTACTGGAGAATGCTGTTCGACCCTGCTAATCTCAAGGAC | |
| | GTCGTGTACCAACTGAACGGCAATGCCGAAATCTTCTTCCGGAAGGCCAGCGTT | |
| | ACAAGGACAGAACCAACACACCCCGCCAATGTGGCCATCAAGAACAAGAGCGAG | |
| | TACAACAAGCAGAACAAACCTTACAGCACCTTCAAGTACGGCCTCATCAAGGACC | |
| | GGCGATACACCACCGATCAGTTCGAGTTCCACGTGCCTATCACCATGAACTTCAA | |
| | GCAACCTGAGTCATCTAAGCTGCAGGACAAACTGAATAAGCAAGTGCTGGACTTC | |
| | CTGAAGCAAGACGGCGTGCGGCACATCATCGGCATCGACCGGGGAGAAAGAAA | |
| | CCTGCTGTACCTGGTGATGGTCGACATGGAAGGAAAAATCAAGAAGCAGATCAG | |
| | CCTGAATGAAATCGCCGGAAACCCAAAGAACCCTGAGTTTAAGCAGGACTTCTTA | |
| | GCTCTGCTGCATGAGAGAGAGGGCGATAGACTGGAGTCCAGAAGAAGTTGGAAC | |
| | ACCATCCAGAGCATCAAGGAGCTGAAAGAAGGCTACATGTCCCTGGTGGTGCAC | |
| | GAGATCGCTAACATGATGCTGGAGAATGATGCCATCGTGGTCTTGGAAAACCTTA | |
| | ACAGATCCTTTATGCAGAAGAGAGGCGGCATTGAGAAAAGCGTGTACCAGAAGT | |
| | TTGAGAAAATGCTGATCGACAAGCTGGGCTACATCGTGGACAAAACAAAAGATGT | |
| | GTCAGATAATGGCGGAGCCCTGCACGCCGTGCAGCTGGCTGACACCTTCGAGAA | |
| | CTTTAACAAGACCCAGAAAGGCGCCATCCGGCAGTGCGGCTTCATCTTTTATATC | |
| | CCCGCCTGGCGGACAAGCAAAATTGACCCGGTAACCGGCTTTGTGCCCATGCTG | |
| | AGATGTCAGTACGAATCTATCGTGGAATCCAAGAAGTTCTTTGGCAAATTCGACT | |
| | CTATCTACTACGACGCCACCGGAAAGTACTTCGTGTTCCAGACCGACTTTACCAA | |
| | GTTCAACACCGAGTCTAAGGGGGGCATCCAGAAGTGGGACATCTGTACCTACGG | |
| | AGACAGAATCTACGCCCCTAGAACCAAAGACAGAAATAACAACCCTGTGTCCGAA | |
| | AGAGTGAACCTGACAGAAGAAATGAAGAGCCTGTTCGTAAGCCACAATATCAACA | |
| | TCCAGGGCGACATCAAGGCCGGCATTATGCAGCAGACAGACAAGGAGTTCTTCG | |
| | AGTCGCTGCACAGACTGCTGAGACTGACCCTGCAGATCCGGAACAGCAAGAAAA | |
| | GCACCGGCAAGGACTACGAGGACTACATTATCAGTCCTGTGATGGGCAAGGACG | |
| | GAAGATTCTTCGACAGCCGGAACGCCGACGCCACCCAGCCCAAGGACGCCGAC | |
| | GCAAACGGCGCCTACAACATTGCCAGAAAAGGCCTGATGCTGCTGCGCCAGATC | |
| | CAGGCCCAGGAGAAGCAGGACCTGTCTAATGGGAAGTGGCTGGAGTTCGCCCA | |
| | GCGG | |
| Expression | ATGggcAAGCGGCTCATCGACTTCACCAACATCTACCAGCGTTCTAAGACCCTGA | 42 |
| construct (with | GATTCAGACTGGAACCTATCGGCAAGACCGCGGACTACATCAAAAACAGCCAGT | |
| N-terminal | CCCTGGAAACAGACGCCAGACTGGCCAAGGAATCCAAGAAAGTGAAGGAACTGG | |
| methionine | CCGATGAGTACCACAAAGAGTTTATCGGCGACGTGCTGAGCAGCCTGGAGCTGC | |
| and stop | CCCTGAGCAAAATCAACGAGCTGTGGGACATCTATATCTACATCTACATGAGCAA | |
| codon, | CGACACCGATCGGGAAATCAAATTTAAGAAGCTCCAGGAGAACCTGCGGAAGGT | |
| includes V5- | GATCGCCGAGGCCTTTAGCAAGGATAAGAGATTCGGCAACCTGTTCAAGAAAGA | |
| tag and C- | AATCATCACAGATATCCTGCCCGAGTTCCTGCAAGATAAAGATGACGATATCAAA | |
| terminal NLS) | ATCGTGAACCGGTTCAAGGGTTTTACAACCTACTTCTACGCCTTCCACAAGAATC | |
| | GGGAAAACATGTACGTGTCTGAAGAGAAGAGCACAGCCATCCCCTACAGAATCG | |
| | TGAATCAAAACCTGGTGAAATACTTCGATAACTACAAGACTTTTAAGGAGAAGGT | |
| | GATGCCTCTGCTGAAGGACAAGAACATCGTCGAAAGCATCGAGCGCGACTTCAA | |
| | GGACATCCTGAACGAGAAAAGCATCGAGGACGTGTTCGGCCTGGCCAATTTCAC | |
| | CCACACCCTGTGCCAGGCTGACATCGAGAAGTACAACACCTTGATAGGCGGACT | |
| | GGTGGTGAAGAACGAAAAGAAGGAGATCAAGGGCATCAACCAGTATATTAACGA | |
| | GCACAACCAGACCTCTAAGAAGGGCAACGGCATCCCAAAGCTGAAGCCTCTGTT | |
| | TAACCAGATCCTGAGCGACAGAAAATCTCTCAGCTTCACCCTGGATGATATCAAG | |
| | AAAACCAGCGAGGCCATCAGAACAATTAAGGACGAGTATGAGAACCTGAGAGAT | |
| | AAGCTGGCCACAATCGAACGGCTGATCAAGAGCATCAAGGAATACGACCTGGCC | |
| | GGCATCTACATCAAGATGGGCGAGGACACCTCTACCATCTCCCAGCACTGGTTC | |
| | GGTGCCTATTACAAGATTATCGAAGCCATCGCCGACGCCTGGGAGAGAAGAAAC | |
| | CCAAAGAAAAACAGAGAGAGCAAGGCCTACAGCAAGTACGTGAGCAGCCTTAAG | |
| | AGCATCAGCCTGCAGGAGATCGACGACCTGAAGATCGGCGAGCCTATCGAGAAT | |
| | TACTTCGCCACCTTTGGAACAACATGTAGCGACCGGACATCTGGCGTGAGCTCT | |
| | CTGAACCGGATCAAAGCCGCCTACACCGAGTTCGTGAACAAGTTCCCCGAGGGC | |
| | TTTGAGGATGGCGATGATTGCAACGACGCTTACTTCAAAGCCAATGTGGAGGTG | |
| | GTGAAGAACTTGCTGGATAGCATAAAAGACTTCCAGAGATTTGTGAAGCCTCTAC | |
| | TGGGCAATGAGGACGAGCGGGACAAAGATGAGGCCTTCTACGGCGAGTTCGTTC | |
| | CTACCTACACAGATATGGACAACATCATCACGCCTCTGTATAATAGAGTCAGAAA | |
| | CTTCGCTACCAAGAAGCCTTACAGTACAGACAAGATCAAAATAAACTTCGAAAAC | |
| | GTGGTACTGCTGAAGGGCTGGGATAAGAACAAGGAGAGCGACTATGCCAGCATC | |
| | ATCCTGATGAAGGACGGCCAGTACTTTCTGGGAGTGCTGAGAAACGGATCTAAG | |
| | AGCACTCTGAAAACCATCCTGCCTAACACCGGTGACTGCTACCAGAAAATGGTGT | |
| | ACAAGTATTTCAAGGATATCAAGTCTAACCTGCCCAGATGCACCACCCAGAGAAA | |
| | GGACGTGAAGGCACATTTCGCTGAAAGCAGCGATGATTACACCCTGCTTGATACA | |
| | AAAGCCTTCGTGAGCGCTCTGACGATCTCCAGAGAGGTGTTCGAACTGTACAAC | |
| | GCTCCTGATAAGGAAAAGAAATTCAAGAAGGAATACCTGAAGAACACCAACGACT | |
| | CCATCGGCTACGCCAATGCAGTGAGCGTGTGCAAGAGATTCTGCCTGGAGTTCC | |
| | TGAAAAAGTACCGGAGCACCGCCATCTACGACCTGAGCGATGTTGAAACCTCTG | |
| | TGGACAGTTTCGACGACCTGAGCAGCTTCTACCAGGAGATCGATAAGAGACTGT | |
| | ACAGCATCAGCTTCGAAAACGTGAGCGTGGACAGCGTGAACGAGCTGGTGGATA | |
| | ACGGCAATATGCTGCTGTTCAGAATCGCCAACAAGGATTTCTCTCCTAATAGCAA | |
| | GGGCAGACCTAATCTGCACACAATTTACTGGAGAATGCTGTTCGACCCTGCTAAT | |
| | CTCAAGGACGTCGTGTACCAACTGAACGGCAATGCCGAAATCTTCTTCCGGAAG | |
| | GCCAGCGTTACAAGGACAGAACCAACACACCCCGCCAATGTGGCCATCAAGAAC | |
| | AAGAGCGAGTACAACAAGCAGAACAAACCTTACAGCACCTTCAAGTACGGCCTCA | |
| | TCAAGGACCGGCGATACACCACCGATCAGTTCGAGTTCCACGTGCCTATCACCA | |
| | TGAACTTCAAGCAACCTGAGTCATCTAAGCTGCAGGACAAACTGAATAAGCAAGT | |
| | GCTGGACTTCCTGAAGCAAGACGGCGTGCGGCACATCATCGGCATCGACCGGG | |
| | GAGAAAGAAACCTGCTGTACCTGGTGATGGTCGACATGGAAGGAAAAATCAAGA | |
| | AGCAGATCAGCCTGAATGAAATCGCCGGAAACCCAAAGAACCCTGAGTTTAAGC | |
| | AGGACTTCTTAGCTCTGCTGCATGAGAGAGAGGGCGATAGACTGGAGTCCAGAA | |
| | GAAGTTGGAACACCATCCAGAGCATCAAGGAGCTGAAAGAAGGCTACATGTCCC | |
| | TGGTGGTGCACGAGATCGCTAACATGATGCTGGAGAATGATGCCATCGTGGTCT | |
| | TGGAAAACCTTAACAGATCCTTTATGCAGAAGAGAGGCGGCATTGAGAAAAGCGT | |
| | GTACCAGAAGTTTGAGAAAATGCTGATCGACAAGCTGGGCTACATCGTGGACAAA | |
| | ACAAAAGATGTGTCAGATAATGGCGGAGCCCTGCACGCCGTGCAGCTGGCTGAC | |
| | ACCTTCGAGAACTTTAACAAGACCCAGAAAGGCGCCATCCGGCAGTGCGGCTTC | |
| | ATCTTTTATATCCCCGCCTGGCGGACAAGCAAAATTGACCCGGTAACCGGCTTTG | |
| | TGCCCATGCTGAGATGTCAGTACGAATCTATCGTGGAATCCAAGAAGTTCTTTGG | |
| | CAAATTCGACTCTATCTACTACGACGCCACCGGAAAGTACTTCGTGTTCCAGACC | |
| | GACTTTACCAAGTTCAACACCGAGTCTAAGGGGGGCATCCAGAAGTGGGACATC | |
| | TGTACCTACGGAGACAGAATCTACGCCCCTAGAACCAAAGACAGAAATAACAACC | |
| | CTGTGTCCGAAAGAGTGAACCTGACAGAAGAAATGAAGAGCCTGTTCGTAAGCC | |
| | ACAATATCAACATCCAGGGCGACATCAAGGCCGGCATTATGCAGCAGACAGACA | |
| | AGGAGTTCTTCGAGTCGCTGCACAGACTGCTGAGACTGACCCTGCAGATCCGGA | |
| | ACAGCAAGAAAAGCACCGGCAAGGACTACGAGGACTACATTATCAGTCCTGTGA | |
| | TGGGCAAGGACGGAAGATTCTTCGACAGCCGGAACGCCGACGCCACCCAGCCC | |
| | AAGGACGCCGACGCAAACGGCGCCTACAACATTGCCAGAAAAGGCCTGATGCTG | |
| | CTGCGCCAGATCCAGGCCCAGGAGAAGCAGGACCTGTCTAATGGGAAGTGGCT | |
| | GGAGTTCGCCCAGCGGtctagaAAGCGGACAGCAGACGGCTCCGAATTTGAAAGC | |
| | CCTAAGAAAAAGAGAAAGGTGggatccGGCAAACCTATCCCCAATCCCCTGCTGGG | |
| | CCTGGACAGCACCTGA | |

In some embodiments a ZKBG Type V Cas protein comprises an amino acid sequence of SEQ ID NO: 37, SEQ ID NO:38, or SEQ ID NO:39. In some embodiments, a ZKBG Type V Cas protein has nickase activity, for example resulting from one or more amino acid substitutions relative to the sequence of SEQ ID NO:37, SEQ ID NO:38, or SEQ ID NO:39. In some embodiments, the one or more amino acid substitutions providing nickase activity comprise a D885 substitution, wherein the position of the D885 substitution is defined with respect to the amino acid numbering of SEQ ID NO:38 (corresponding to amino acid 908 of SEQ ID NO: 121). In some embodiments, the one or more amino acid substitutions providing nickase activity comprise an E978 substitution, wherein the position of the E978 substitution is defined with respect to the amino acid numbering of SEQ ID NO:38 (corresponding to amino acid 993 of SEQ ID NO: 121). In some embodiments, the one or more amino acid substitutions providing nickase activity comprise a R1194 substitution, wherein the position of the R1194 substitution is defined with respect to the amino acid numbering of SEQ ID NO:38 (corresponding to amino acid 1226 of SEQ ID NO: 121). In some embodiments, the one or more amino acid substitutions providing nickase activity comprise a D1234 substitution, wherein the position of the D1234 substitution is defined with respect to the amino acid numbering of SEQ ID NO:38 (corresponding to amino acid 1263 of SEQ ID NO:121). In some embodiments, a ZKBG Type V Cas protein is catalytically inactive, for example due to a R1194 substitution in combination with a D885 substitution, a E978 substitution, and/or D1234 substitution.

### 6.2.8. ZZKD Type V Cas Proteins

In one aspect, the disclosure provides ZZKD Type V Cas proteins. ZZKD Type V Cas proteins can be further classified as Type V-A Cas proteins. The ZZKD Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:43. In some embodiments, the ZZKD Type V Cas proteins comprise an amino acid sequence that is at least 95%, at least 96%, at least 97%, at least 98%, or at least 99% identical to SEQ ID NO:43. In some embodiments, a ZZKD Type V Cas protein comprises an amino acid sequence that is identical to SEQ ID NO:43.

Exemplary ZZKD Type V Cas protein sequences and nucleotide sequences encoding exemplary ZZKD Type V Cas proteins are set forth in Table 1H.

TABLE 1H
ZZKD Type V Cas Sequences

| | | SEQ ID |
|---|---|---|
| Name | Sequence | NO. |
| Wildtype | AEMFKDFTNLYPVSKTLRFELIPEGETLHYLEKNGVLENDEKRNEDYKKLKKLMDEY | 43 |
| amino acid | YRAYIDEALSNVHLSDLDRYAELYSIQNKSDEENVEFENVQLRLRTQIVGFLESRETY | |
| sequence | SSLFKKELIEKELPKFFIRREEELNLIKSFKGFTTMCTGFWENRKNMFSAEEKSTAIA | |
| (without N- | YRVVHENLPKFMNNIRIFRLFIDEKLDCSEKLLEKAGVNSLSEVFELDYFNNTLSQRG | |
| terminal | IELYNCILGGFTEDEKHKIQGVNELINLYNQQTKEKKIPQLQPLYKQILSDTKSLSFLA | |
| methionine) | DAFENDGGVLATVKALYDEFHEEILSERGLISTTLQNIEKYDSKGIFVKNDLTITGLSN | |
| | SLFGDWKAINGSLNSWYEENVPRKERTEEKHVEVRKAYFKKLKSISLEFIEEAGLSE | |
| | LRCKYKALLLEKAEAVCDAYKNAEELFSEAYNENTNLIADGKSVEKIKALLDSMKELE | |
| | AVILMLSGTGEEAERDELFYGEFEKHRFVLNLLDNVFNKTRNYVTKKPYKTEKIKLTF | |
| | DSPTLLDGWDRNKETSNKSVILMKDGYYYLGIMNKANNKAFENLKDTGGKCYSKM | |
| | DYKLLPGPNKMLPKVFFAKKNIDYYAPSEDLLQKYKEGTHKKGKKFNLEDCHALIDF | |
| | FKDSIAKHPEWNEFGFDFSDTKSYRDISDFYKEVSEQGYKISYRNVSVNYIDSLVRE | |
| | GKLYLFKIYNKDFSPYSKGRPNLHTMYWKALFANKNFENRIYKLNGQAEMFYRKKSI | |
| | PEDKRVIHSAKEPIDQRRNTDEKSLFDYDIIKDRRYTVDKFQFNVPITMNYTAPGSGR | |
| | INRKMREAIKNCENMHIIGIDRGERHLLYVTVIDMQGNIKEQFSLNRILSEYKANNVAK | |
| | SVETDYKTLLTKKEIERQDARKQWKSIENIKELKDGYMSQVVHVIAELMIKYNAIVVM | |
| | EDLNFGFKRGRQKVERQVYQKFEKALIDKLNYLVDKTASEMENTGLYAALQLTEKF | |
| | ESFKKMGKQNGGLFYVNAWNTSKMDPTTGFVNLLYPKYESIEKSKAYIEKFKDIQFC | |
| | DDDEYGKYLAISFDYNDFTEKAKGAKTEWTICSYGKRLYNHRNKDGYWEEQELDLT | |
| | EEYFNLFEEFGINAASNIKEQVIAQNSADFFRRFMWLLKMTLQIRNSETNGETDYML | |
| | SPVKNEDGKFFNSDEVKDDTLPENADANGAYNIARKGLLLVERIKDCPDEELDKVDL | |
| | KVTNLDWMKFAQR | |
| Wildtype | MAEMFKDFTNLYPVSKTLRFELIPEGETLHYLEKNGVLENDEKRNEDYKKLKKLMDE | 44 |
| amino acid | YYRAYIDEALSNVHLSDLDRYAELYSIQNKSDEENVEFENVQLRLRTQIVGFLESRET | |
| sequence (with | YSSLFKKELIEKELPKFFIRREEELNLIKSFKGFTTMCTGFWENRKNMFSAEEKSTAI | |
| N-terminal | AYRVVHENLPKFMNNIRIFRLFIDEKLDCSEKLLEKAGVNSLSEVFELDYFNNTLSQR | |
| methionine) | GIELYNCILGGFTEDEKHKIQGVNELINLYNQQTKEKKIPQLQPLYKQILSDTKSLSFL | |
| | ADAFENDGGVLATVKALYDEFHEEILSERGLISTTLQNIEKYDSKGIFVKNDLTITGLS | |
| | NSLFGDWKAINGSLNSWYEENVPRKERTEEKHVEVRKAYFKKLKSISLEFIEEAGLS | |
| | ELRCKYKALLLEKAEAVCDAYKNAEELFSEAYNENTNLIADGKSVEKIKALLDSMKEL | |
| | EAVILMLSGTGEEAERDELFYGEFEKHRFVLNLLDNVFNKTRNYVTKKPYKTEKIKLT | |
| | FDSPTLLDGWDRNKETSNKSVILMKDGYYYLGIMNKANNKAFENLKDTGGKCYSKM | |
| | DYKLLPGPNKMLPKVFFAKKNIDYYAPSEDLLQKYKEGTHKKGKKFNLEDCHALIDF | |
| | FKDSIAKHPEWNEFGFDFSDTKSYRDISDFYKEVSEQGYKISYRNVSVNYIDSLVRE | |
| | GKLYLFKIYNKDFSPYSKGRPNLHTMYWKALFANKNFENRIYKLNGQAEMFYRKKSI | |
| | PEDKRVIHSAKEPIDQRRNTDEKSLFDYDIIKDRRYTVDKFQFNVPITMNYTAPGSGR | |
| | INRKMREAIKNCENMHIIGIDRGERHLLYVTVIDMQGNIKEQFSLNRILSEYKANNVAK | |
| | SVETDYKTLLTKKEIERQDARKQWKSIENIKELKDGYMSQVVHVIAELMIKYNAIVVM | |
| | EDLNFGFKRGRQKVERQVYQKFEKALIDKLNYLVDKTASEMENTGLYAALQLTEKF | |
| | ESFKKMGKQNGGLFYVNAWNTSKMDPTTGFVNLLYPKYESIEKSKAYIEKFKDIQFC | |
| | DDDEYGKYLAISFDYNDFTEKAKGAKTEWTICSYGKRLYNHRNKDGYWEEQELDLT | |
| | EEYFNLFEEFGINAASNIKEQVIAQNSADFFRRFMWLLKMTLQIRNSETNGETDYML | |
| | SPVKNEDGKFFNSDEVKDDTLPENADANGAYNIARKGLLLVERIKDCPDEELDKVDL | |
| | KVTNLDWMKFAQR | |
| Expression | MGAEMFKDFTNLYPVSKTLRFELIPEGETLHYLEKNGVLENDEKRNEDYKKLKKLM | 45 |
| construct (with | DEYYRAYIDEALSNVHLSDLDRYAELYSIQNKSDEENVEFENVQLRLRTQIVGFLES | |
| N-terminal | RETYSSLFKKELIEKELPKFFIRREEELNLIKSFKGFTTMCTGFWENRKNMFSAEEKS | |
| methionine, | TAIAYRVVHENLPKFMNNIRIFRLFIDEKLDCSEKLLEKAGVNSLSEVFELDYFNNTLS | |
| V5-tag and C- | QRGIELYNCILGGFTEDEKHKIQGVNELINLYNQQTKEKKIPQLQPLYKQILSDTKSLS | |
| terminal NLS) | FLADAFENDGGVLATVKALYDEFHEEILSERGLISTTLQNIEKYDSKGIFVKNDLTITG | |
| aa sequence | LSNSLFGDWKAINGSLNSWYEENVPRKERTEEKHVEVRKAYFKKLKSISLEFIEEAG | |
| | LSELRCKYKALLLEKAEAVCDAYKNAEELFSEAYNENTNLIADGKSVEKIKALLDSMK | |
| | ELEAVILMLSGTGEEAERDELFYGEFEKHRFVLNLLDNVFNKTRNYVTKKPYKTEKIK | |
| | LTFDSPTLLDGWDRNKETSNKSVILMKDGYYYLGIMNKANNKAFENLKDTGGKCYS | |
| | KMDYKLLPGPNKMLPKVFFAKKNIDYYAPSEDLLQKYKEGTHKKGKKFNLEDCHALI | |
| | DFFKDSIAKHPEWNEFGFDFSDTKSYRDISDFYKEVSEQGYKISYRNVSVNYIDSLV | |
| | REGKLYLFKIYNKDFSPYSKGRPNLHTMYWKALFANKNFENRIYKLNGQAEMFYRK | |
| | KSIPEDKRVIHSAKEPIDQRRNTDEKSLFDYDIIKDRRYTVDKFQFNVPITMNYTAPG | |
| | SGRINRKMREAIKNCENMHIIGIDRGERHLLYVTVIDMQGNIKEQFSLNRILSEYKAN | |
| | NVAKSVETDYKTLLTKKEIERQDARKQWKSIENIKELKDGYMSQVVHVIAELMIKYNA | |
| | IVVMEDLNFGFKRGRQKVERQVYQKFEKALIDKLNYLVDKTASEMENTGLYAALQLT | |
| | EKFESFKKMGKQNGGLFYVNAWNTSKMDPTTGFVNLLYPKYESIEKSKAYIEKFKDI | |
| | QFCDDDEYGKYLAISFDYNDFTEKAKGAKTEWTICSYGKRLYNHRNKDGYWEEQE | |
| | LDLTEEYFNLFEEFGINAASNIKEQVIAQNSADFFRRFMWLLKMTLQIRNSETNGETD | |
| | YMLSPVKNEDGKFFNSDEVKDDTLPENADANGAYNIARKGLLLVERIKDCPDEELDK | |
| | VDLKVTNLDWMKFAQRSRKRTADGSEFESPKKKRKVGSGKPIPNPLLGLDST | |
| Wildtype | ATGGCTGAGATGTTTAAAGATTTTACGAATTTGTATCCTGTTTCAAAAACCTTGC | 46 |
| coding | GTTTTGAATTAATTCCTGAAGGGGAAACATTGCATTATCTTGAAAAAAATGGCGT | |
| sequence (with | TCTGGAAAACGATGAGAAGCGAAACGAAGATTATAAGAAGTTGAAAAAACTGAT | |
| N-terminal | GGATGAATATTACCGTGCATACATCGATGAAGCTTTATCTAATGTTCATCTTTCA | |
| methionine | GATTTGGATAGATATGCAGAATTATATTCAATTCAGAATAAATCGGATGAAGAAA | |
| and stop | ATGTAGAATTCGAAAATGTTCAACTGAGATTGAGAACACAAATTGTTGGATTCTT | |
| codon) | AGAATCCAGAGAAACCTATTCTTCACTTTTCAAAAAAGAACTGATTGAGAAGGAA | |
| | CTTCCTAAATTCTTTATTCGGAGAGAAGAGGAGCTTAATTTAATCAAATCATTTAA | |
| | AGGTTTTACAACGATGTGCACCGGCTTCTGGGAAAATCGGAAAAATATGTTTTCT | |
| | GCCGAAGAAAAATCTACAGCAATAGCATATCGTGTAGTCCATGAAAACCTACCTA | |
| | AGTTTATGAATAATATAAGAATTTTTCGTTTGTTCATTGATGAAAAGTTGGACTGT | |
| | TCTGAAAAATTGCTGGAAAAAGCCGGAGTGAATTCTCTGAGTGAAGTGTTTGAA | |
| | CTTGATTATTTTAACAATACATTATCCCAACGTGGCATTGAATTGTATAACTGTAT | |
| | ATTGGGCGGATTTACCGAGGATGAAAAGCATAAGATTCAAGGCGTAAACGAATT | |
| | GATTAATTTGTACAATCAGCAGACAAAAGAGAAGAAGATTCCACAGTTGCAGCC | |
| | GCTGTACAAGCAGATTCTCAGCGATACCAAGAGCCTTTCATTTCTTGCAGATGC | |
| | ATTTGAAAACGACGGGGGGGTCTTAGCGACTGTAAAAGCATTATATGATGAATTT | |
| | CATGAAGAGATTTTGAGCGAAAGGGGATTAATCTCTACGACATTACAGAATATTG | |
| | AAAAGTATGATTCAAAAGGCATCTTCGTAAAAAACGATTTAACGATTACCGGTTT | |
| | ATCAAATAGTTTGTTCGGCGACTGGAAGGCTATTAATGGTAGTTTAAATTCGTGG | |
| | TATGAGGAGAACGTGCCTCGAAAAGAAAGAACTGAAGAGAAACATGTAGAGGTA | |
| | AGAAAAGCCTATTTTAAAAAGTTAAAATCAATAAGCCTGGAATTTATCGAGGAGG | |
| | CCGGATTGTCGGAACTCCGTTGCAAATATAAAGCCCTTCTTTTAGAAAAAGCAGA | |
| | GGCTGTTTGCGATGCGTACAAAAATGCAGAAGAGCTTTTTAGTGAAGCTTATAAT | |
| | GAAAATACTAACCTTATTGCCGATGGAAAGTCTGTGGAAAAAATAAAAGCGCTAT | |
| | TGGATTCTATGAAAGAGCTTGAAGCGGTGATTCTTATGCTTTCCGGAACCGGAG | |
| | AGGAAGCAGAACGGGATGAATTGTTTTACGGCGAATTTGAAAAACATAGGTTCG | |
| | TATTGAATCTCTTAGACAACGTATTTAATAAAACGAGAAATTACGTAACAAAGAAA | |
| | CCATATAAGACTGAGAAGATTAAATTAACATTTGATTCCCCAACGCTGCTAGACG | |
| | GGTGGGATCGTAATAAAGAAACATCAAACAAGTCCGTGATACTTATGAAAGATG | |
| | GCTATTATTACCTTGGAATTATGAACAAGGCAAATAACAAAGCCTTTGAGAATTT | |
| | GAAAGACACAGGCGGGAAATGCTATAGCAAGATGGATTACAAACTTTTGCCTGG | |
| | ACCAAACAAGATGTTGCCGAAGGTGTTTTTTGCAAAGAAAAACATCGACTATTAT | |
| | GCACCAAGCGAAGACTTGCTACAGAAATATAAAGAGGGAACACATAAAAAAGGA | |
| | AAGAAATTTAATCTAGAGGATTGTCACGCGTTAATAGACTTTTTTAAAGACTCAAT | |
| | TGCAAAGCATCCAGAATGGAACGAGTTTGGATTTGATTTTTCAGATACGAAATCA | |
| | TATCGAGATATTAGTGATTTCTATAAGGAGGTTTCAGAGCAGGGATACAAAATCA | |
| | GTTATCGAAATGTATCTGTTAATTACATAGATTCTCTAGTAAGAGAAGGGAAATT | |
| | GTATTTGTTCAAAATTTATAATAAAGATTTTTCACCGTACAGCAAAGGCAGACCAA | |
| | ATCTTCATACGATGTATTGGAAAGCGTTATTCGCTAATAAGAATTTTGAAAATCG | |
| | CATATATAAGTTAAATGGCCAGGCAGAAATGTTCTATCGAAAAAAGAGCATTCCG | |
| | GAAGACAAGAGGGTGATTCACTCGGCAAAAGAACCAATCGATCAGAGAAGAAAT | |
| | ACGGATGAAAAGAGCCTCTTTGATTATGACATTATTAAAGATCGGCGATATACTG | |
| | TGGACAAATTCCAATTTAATGTTCCGATTACGATGAATTACACTGCACCGGGTTC | |
| | CGGCCGAATTAACAGAAAAATGCGGGAAGCGATTAAGAACTGTGAAAATATGCA | |
| | TATTATCGGAATAGATAGAGGCGAACGTCATTTGCTGTATGTGACGGTTATCGAT | |
| | ATGCAGGGAAACATTAAAGAACAGTTTTCATTAAATCGAATCCTGAGTGAGTACA | |
| | AGGCAAACAATGTGGCTAAAAGTGTCGAAACGGACTACAAAACACTCCTGACAA | |
| | AAAAAGAAATTGAACGACAGGATGCAAGAAAGCAGTGGAAGAGCATTGAAAATA | |
| | TTAAGGAATTAAAAGACGGCTACATGAGCCAGGTTGTGCATGTGATTGCCGAAC | |
| | TCATGATAAAGTACAATGCGATTGTGGTTATGGAGGATTTGAATTTCGGATTCAA | |
| | GCGAGGAAGACAGAAGGTTGAGAGACAGGTTTACCAGAAGTTTGAGAAGGCAT | |
| | TAATTGATAAATTGAACTATTTGGTTGATAAAACAGCCTCTGAAATGGAGAACAC | |
| | CGGTCTGTATGCGGCATTGCAGCTTACAGAAAAATTTGAGAGCTTTAAGAAAAT | |
| | GGGCAAACAAAATGGTGGATTATTTTATGTAAACGCATGGAATACCAGTAAAATG | |
| | GATCCAACAACCGGTTTTGTGAACCTTCTCTATCCTAAATATGAGAGCATTGAAA | |
| | AAAGCAAAGCGTATATTGAGAAATTCAAGGATATTCAGTTTTGTGATGATGACGA | |
| | ATATGGAAAGTACCTTGCAATATCTTTTGATTATAACGATTTCACGGAGAAGGCA | |
| | AAGGGCGCAAAAACGGAATGGACCATTTGCTCTTATGGAAAGAGATTGTATAAT | |
| | CACAGAAATAAAGATGGGTATTGGGAAGAGCAGGAATTGGATCTTACAGAAGAG | |
| | TATTTCAATCTGTTTGAAGAATTTGGAATTAATGCAGCGTCTAATATTAAAGAACA | |
| | AGTCATCGCACAGAATTCTGCAGACTTTTTTAGACGGTTTATGTGGCTTTTGAAA | |
| | ATGACCTTACAGATTAGAAACAGTGAAACAAATGGGGAGACGGATTATATGCTTT | |
| | CTCCGGTAAAAAATGAAGACGGAAAATTCTTTAATTCAGATGAAGTCAAGGATGA | |
| | CACGCTTCCGGAAAATGCGGATGCGAATGGTGCATACAACATCGCTAGAAAAG | |
| | GATTACTGCTTGTGGAAAGAATTAAAGACTGTCCGGACGAAGAACTTGATAAGG | |
| | TTGATTTGAAGGTAACAAATTTAGATTGGATGAAATTTGCACAGAGGTAA | |
| Codon | GCCGAAATGTTCAAGGACTTCACCAACCTGTACCCAGTGTCCAAAACCCTCCGG | 47 |
| optimized | TTCGAATTGATCCCCGAGGGCGAAACACTGCACTACCTAGAAAAGAACGGAGTG | |
| coding | CTGGAAAACGACGAGAAGAGAAATGAGGATTACAAGAAGCTGAAGAAACTCATG | |
| sequence (no | GATGAATACTACCGGGCCTACATCGACGAGGCCTTATCTAATGTCCACCTGTCC | |
| N-terminal | GATCTGGACCGGTACGCCGAACTGTATTCTATCCAGAACAAGAGCGATGAGGA | |
| methionine, no | GAACGTGGAGTTCGAGAATGTGCAGCTGCGCCTGAGAACCCAGATCGTGGGCT | |
| stop codon) | TCCTGGAAAGCAGAGAAACCTACAGCAGCCTGTTCAAGAAGGAGCTGATCGAAA | |
| | AAGAACTGCCTAAGTTTTTCATCAGAAGAGAGGAAGAGCTGAACCTGATAAAGA | |
| | GCTTTAAGGGCTTTACCACTATGTGCACCGGCTTCTGGGAAAATCGGAAGAACA | |
| | TGTTCAGCGCCGAGGAAAAGTCCACAGCCATCGCCTATAGAGTGGTCCATGAAA | |
| | ACCTGCCCAAGTTCATGAACAACATTAGAATCTTCCGGCTGTTTATCGACGAGAA | |
| | GCTGGATTGTAGCGAGAAGCTGCTGGAGAAGGCCGGCGTGAACAGCCTGAGC | |
| | GAGGTGTTCGAGCTTGACTATTTCAATAACACCCTGAGCCAGAGAGGCATCGAG | |
| | CTGTACAACTGCATCCTGGGCGGATTCACCGAGGATGAAAAACACAAGATCCAG | |
| | GGAGTGAACGAGTTGATCAACCTGTACAACCAGCAGACAAAGGAGAAGAAAATT | |
| | CCTCAGCTGCAACCTCTGTACAAACAGATCCTGTCTGACACGAAGTCGCTGTCC | |
| | TTTCTGGCTGATGCCTTTGAAAACGACGGAGGAGTGCTGGCTACAGTGAAGGCT | |
| | TTATATGATGAGTTTCACGAGGAAATCCTGAGCGAGAGAGGCCTGATCAGCACA | |
| | ACCCTGCAGAACATTGAGAAGTACGATAGTAAGGGCATCTTTGTTAAGAACGAT | |
| | CTCACCATTACAGGCCTGTCCAACAGCCTGTTTGGAGATTGGAAGGCCATCAAT | |
| | GGAAGCCTGAACAGCTGGTACGAGGAGAACGTGCCCCGGAAGGAGCGAACAG | |
| | AAGAGAAACACGTGGAAGTGAGAAAGGCTTATTTTAAGAAGCTGAAGTCTATCA | |
| | GCCTGGAGTTCATCGAGGAGGCCGGACTGAGCGAGCTGCGGTGCAAGTACAA | |
| | GGCCCTGCTGCTGGAGAAAGCCGAGGCTGTGTGCGACGCGTACAAGAACGCC | |
| | GAGGAGCTGTTTAGCGAGGCCTATAATGAGAACACCAATCTGATCGCCGATGG | |
| | CAAATCTGTGGAAAAAATCAAAGCCCTGCTGGACAGCATGAAGGAGCTGGAGG | |
| | CCGTGATCCTGATGCTGAGCGGCACAGGCGAGGAGGCCGAGCGGGACGAACT | |
| | GTTTTATGGCGAGTTCGAAAAACATAGATTCGTGCTGAATCTGCTGGACAACGT | |
| | GTTCAACAAGACCAGAAACTACGTGACCAAGAAGCCTTACAAGACCGAGAAGAT | |
| | CAAGCTCACCTTCGACAGCCCTACCCTTCTGGATGGCTGGGACCGTAACAAGG | |
| | AGACAAGCAACAAGAGCGTGATCCTGATGAAGGATGGCTACTACTACCTGGGC | |
| | ATCATGAACAAAGCCAACAACAAGGCCTTCGAGAACCTGAAGGACACAGGAGG | |
| | CAAATGCTACAGCAAGATGGACTACAAGCTGCTGCCTGGCCCTAACAAGATGCT | |
| | GCCTAAGGTGTTCTTTGCCAAAAAGAACATCGACTACTACGCCCCTAGCGAGGA | |
| | CCTGCTGCAGAAGTACAAGGAGGGCACCCACAAGAAAGGGAAGAAGTTCAATC | |
| | TTGAGGACTGTCACGCCCTGATCGACTTCTTCAAGGACAGCATCGCTAAACACC | |
| | CCGAGTGGAACGAGTTCGGCTTCGACTTTTCTGACACCAAGTCTTATAGAGACA | |
| | TCTCGGATTTCTACAAGGAGGTCAGCGAACAGGGCTACAAGATTAGCTACCGGA | |
| | ACGTGAGTGTTAACTACATCGACAGTCTGGTGCGGGAAGGTAAGCTGTACCTGT | |
| | TCAAGATCTACAACAAGGACTTCAGCCCATACTCCAAAGGACGTCCCAACCTGC | |
| | ACACCATGTACTGGAAAGCCCTGTTCGCCAATAAAAACTTCGAAAACCGGATCT | |
| | ACAAGCTGAACGGCCAGGCCGAAATGTTCTACAGAAAGAAATCTATCCCTGAAG | |
| | ATAAGCGGGTGATCCACAGCGCCAAAGAACCTATCGATCAGAGAAGAAACACC | |
| | GACGAAAAGTCTCTGTTTGACTACGACATCATCAAGGACAGACGGTACACCGTG | |
| | GACAAGTTCCAGTTCAACGTGCCAATCACAATGAACTACACCGCCCCTGGCAGC | |
| | GGCAGAATCAACAGAAAGATGCGGGAAGCTATCAAGAATTGCGAGAATATGCAC | |
| | ATCATCGGCATCGACCGGGGAGAGCGGCACCTGCTGTACGTGACCGTGATCGA | |
| | CATGCAGGGCAACATCAAAGAACAGTTCTCTCTCAACCGCATCCTGTCTGAGTA | |
| | CAAGGCCAATAACGTCGCCAAGAGCGTGGAGACAGACTACAAAACACTGCTGA | |
| | CGAAAAAAGAGATCGAGAGACAGGACGCTAGAAAGCAATGGAAGAGCATCGAA | |
| | AACATCAAAGAGCTGAAAGACGGCTATATGAGCCAGGTGGTGCACGTGATAGC | |
| | AGAGCTGATGATCAAGTACAACGCCATAGTTGTGATGGAGGACCTGAATTTCGG | |
| | CTTCAAGAGAGGCCGGCAAAAGGTGGAGAGACAGGTGTACCAGAAATTCGAGA | |
| | AGGCCCTGATCGATAAGCTGAATTACCTGGTGGATAAGACAGCTTCCGAGATGG | |
| | AAAACACCGGCCTGTACGCCGCCCTGCAGCTGACAGAGAAGTTCGAATCCTTC | |
| | AAGAAGATGGGCAAACAGAACGGCGGCTTGTTCTACGTGAACGCCTGGAACAC | |
| | CAGCAAGATGGACCCTACCACCGGATTCGTGAACCTGCTGTACCCTAAGTACGA | |
| | ATCTATCGAAAAGAGCAAGGCCTATATCGAGAAATTCAAGGATATCCAGTTTTGT | |
| | GACGACGATGAATACGGCAAATACCTGGCAATTTCTTTCGACTACAACGACTTC | |
| | ACAGAAAAGGCCAAGGGCGCCAAGACCGAGTGGACCATCTGCAGCTACGGCAA | |
| | AAGACTGTACAACCACAGAAATAAGGACGGCTACTGGGAGGAGCAGGAGCTGG | |
| | ATCTGACCGAGGAGTACTTCAACCTGTTCGAAGAGTTCGGCATCAACGCTGCCA | |
| | GCAACATCAAGGAACAAGTGATCGCTCAGAACAGCGCCGATTTCTTCAGAAGAT | |
| | TCATGTGGCTGCTGAAGATGACCCTGCAGATCAGGAACTCTGAAACTAACGGCG | |
| | AAACCGATTACATGCTGAGCCCTGTGAAGAACGAGGACGGCAAATTCTTCAACT | |
| | CTGACGAGGTGAAGGACGACACCCTGCCCGAGAATGCCGACGCCAACGGCGC | |
| | CTACAACATCGCAAGAAAGGGCCTGCTGCTGGTCGAACGTATCAAGGATTGCC | |
| | CCGACGAAGAACTAGACAAGGTGGACCTGAAGGTCACCAACCTGGACTGGATG | |
| | AAATTCGCCCAAAGA | |
| Expression | ATGggcGCCGAAATGTTCAAGGACTTCACCAACCTGTACCCAGTGTCCAAAACCC | 48 |
| construct (with | TCCGGTTCGAATTGATCCCCGAGGGCGAAACACTGCACTACCTAGAAAAGAACG | |
| N-terminal | GAGTGCTGGAAAACGACGAGAAGAGAAATGAGGATTACAAGAAGCTGAAGAAA | |
| methionine | CTCATGGATGAATACTACCGGGCCTACATCGACGAGGCCTTATCTAATGTCCAC | |
| and stop | CTGTCCGATCTGGACCGGTACGCCGAACTGTATTCTATCCAGAACAAGAGCGAT | |
| codon, | GAGGAGAACGTGGAGTTCGAGAATGTGCAGCTGCGCCTGAGAACCCAGATCGT | |
| includes V5- | GGGCTTCCTGGAAAGCAGAGAAACCTACAGCAGCCTGTTCAAGAAGGAGCTGA | |
| tag and C- | TCGAAAAAGAACTGCCTAAGTTTTTCATCAGAAGAGAGGAAGAGCTGAACCTGA | |
| terminal NLS) | TAAAGAGCTTTAAGGGCTTTACCACTATGTGCACCGGCTTCTGGGAAAATCGGA | |
| | AGAACATGTTCAGCGCCGAGGAAAAGTCCACAGCCATCGCCTATAGAGTGGTC | |
| | CATGAAAACCTGCCCAAGTTCATGAACAACATTAGAATCTTCCGGCTGTTTATCG | |
| | ACGAGAAGCTGGATTGTAGCGAGAAGCTGCTGGAGAAGGCCGGCGTGAACAG | |
| | CCTGAGCGAGGTGTTCGAGCTTGACTATTTCAATAACACCCTGAGCCAGAGAGG | |
| | CATCGAGCTGTACAACTGCATCCTGGGCGGATTCACCGAGGATGAAAAACACAA | |
| | GATCCAGGGAGTGAACGAGTTGATCAACCTGTACAACCAGCAGACAAAGGAGA | |
| | AGAAAATTCCTCAGCTGCAACCTCTGTACAAACAGATCCTGTCTGACACGAAGT | |
| | CGCTGTCCTTTCTGGCTGATGCCTTTGAAAACGACGGAGGAGTGCTGGCTACA | |
| | GTGAAGGCTTTATATGATGAGTTTCACGAGGAAATCCTGAGCGAGAGAGGCCTG | |
| | ATCAGCACAACCCTGCAGAACATTGAGAAGTACGATAGTAAGGGCATCTTTGTT | |
| | AAGAACGATCTCACCATTACAGGCCTGTCCAACAGCCTGTTTGGAGATTGGAAG | |
| | GCCATCAATGGAAGCCTGAACAGCTGGTACGAGGAGAACGTGCCCCGGAAGGA | |
| | GCGAACAGAAGAGAAACACGTGGAAGTGAGAAAGGCTTATTTTAAGAAGCTGAA | |
| | GTCTATCAGCCTGGAGTTCATCGAGGAGGCCGGACTGAGCGAGCTGCGGTGCA | |
| | AGTACAAGGCCCTGCTGCTGGAGAAAGCCGAGGCTGTGTGCGACGCGTACAAG | |
| | AACGCCGAGGAGCTGTTTAGCGAGGCCTATAATGAGAACACCAATCTGATCGCC | |
| | GATGGCAAATCTGTGGAAAAAATCAAAGCCCTGCTGGACAGCATGAAGGAGCT | |
| | GGAGGCCGTGATCCTGATGCTGAGCGGCACAGGCGAGGAGGCCGAGCGGGAC | |
| | GAACTGTTTTATGGCGAGTTCGAAAAACATAGATTCGTGCTGAATCTGCTGGAC | |
| | AACGTGTTCAACAAGACCAGAAACTACGTGACCAAGAAGCCTTACAAGACCGAG | |
| | AAGATCAAGCTCACCTTCGACAGCCCTACCCTTCTGGATGGCTGGGACCGTAAC | |
| | AAGGAGACAAGCAACAAGAGCGTGATCCTGATGAAGGATGGCTACTACTACCTG | |
| | GGCATCATGAACAAAGCCAACAACAAGGCCTTCGAGAACCTGAAGGACACAGG | |
| | AGGCAAATGCTACAGCAAGATGGACTACAAGCTGCTGCCTGGCCCTAACAAGAT | |
| | GCTGCCTAAGGTGTTCTTTGCCAAAAAGAACATCGACTACTACGCCCCTAGCGA | |
| | GGACCTGCTGCAGAAGTACAAGGAGGGCACCCACAAGAAAGGGAAGAAGTTCA | |
| | ATCTTGAGGACTGTCACGCCCTGATCGACTTCTTCAAGGACAGCATCGCTAAAC | |
| | ACCCCGAGTGGAACGAGTTCGGCTTCGACTTTTCTGACACCAAGTCTTATAGAG | |
| | ACATCTCGGATTTCTACAAGGAGGTCAGCGAACAGGGCTACAAGATTAGCTACC | |
| | GGAACGTGAGTGTTAACTACATCGACAGTCTGGTGCGGGAAGGTAAGCTGTAC | |
| | CTGTTCAAGATCTACAACAAGGACTTCAGCCCATACTCCAAAGGACGTCCCAAC | |
| | CTGCACACCATGTACTGGAAAGCCCTGTTCGCCAATAAAAACTTCGAAAACCGG | |
| | ATCTACAAGCTGAACGGCCAGGCCGAAATGTTCTACAGAAAGAAATCTATCCCT | |
| | GAAGATAAGCGGGTGATCCACAGCGCCAAAGAACCTATCGATCAGAGAAGAAA | |
| | CACCGACGAAAAGTCTCTGTTTGACTACGACATCATCAAGGACAGACGGTACAC | |
| | CGTGGACAAGTTCCAGTTCAACGTGCCAATCACAATGAACTACACCGCCCCTGG | |
| | CAGCGGCAGAATCAACAGAAAGATGCGGGAAGCTATCAAGAATTGCGAGAATAT | |
| | GCACATCATCGGCATCGACCGGGGAGAGCGGCACCTGCTGTACGTGACCGTGA | |
| | TCGACATGCAGGGCAACATCAAAGAACAGTTCTCTCTCAACCGCATCCTGTCTG | |
| | AGTACAAGGCCAATAACGTCGCCAAGAGCGTGGAGACAGACTACAAAACACTG | |
| | CTGACGAAAAAAGAGATCGAGAGACAGGACGCTAGAAAGCAATGGAAGAGCAT | |
| | CGAAAACATCAAAGAGCTGAAAGACGGCTATATGAGCCAGGTGGTGCACGTGA | |
| | TAGCAGAGCTGATGATCAAGTACAACGCCATAGTTGTGATGGAGGACCTGAATT | |
| | TCGGCTTCAAGAGAGGCCGGCAAAAGGTGGAGAGACAGGTGTACCAGAAATTC | |
| | GAGAAGGCCCTGATCGATAAGCTGAATTACCTGGTGGATAAGACAGCTTCCGAG | |
| | ATGGAAAACACCGGCCTGTACGCCGCCCTGCAGCTGACAGAGAAGTTCGAATC | |
| | CTTCAAGAAGATGGGCAAACAGAACGGCGGCTTGTTCTACGTGAACGCCTGGA | |
| | ACACCAGCAAGATGGACCCTACCACCGGATTCGTGAACCTGCTGTACCCTAAGT | |
| | ACGAATCTATCGAAAAGAGCAAGGCCTATATCGAGAAATTCAAGGATATCCAGTT | |
| | TTGTGACGACGATGAATACGGCAAATACCTGGCAATTTCTTTCGACTACAACGA | |
| | CTTCACAGAAAAGGCCAAGGGCGCCAAGACCGAGTGGACCATCTGCAGCTACG | |
| | GCAAAAGACTGTACAACCACAGAAATAAGGACGGCTACTGGGAGGAGCAGGAG | |
| | CTGGATCTGACCGAGGAGTACTTCAACCTGTTCGAAGAGTTCGGCATCAACGCT | |
| | GCCAGCAACATCAAGGAACAAGTGATCGCTCAGAACAGCGCCGATTTCTTCAGA | |
| | AGATTCATGTGGCTGCTGAAGATGACCCTGCAGATCAGGAACTCTGAAACTAAC | |
| | GGCGAAACCGATTACATGCTGAGCCCTGTGAAGAACGAGGACGGCAAATTCTT | |
| | CAACTCTGACGAGGTGAAGGACGACACCCTGCCCGAGAATGCCGACGCCAACG | |
| | GCGCCTACAACATCGCAAGAAAGGGCCTGCTGCTGGTCGAACGTATCAAGGAT | |
| | TGCCCCGACGAAGAACTAGACAAGGTGGACCTGAAGGTCACCAACCTGGACTG | |
| | GATGAAATTCGCCCAAAGGtctagaAAGCGGACAGCAGACGGCTCCGAATTTGAA | |
| | AGCCCTAAGAAAAAGAGAAAGGTGggatccGGCAAACCTATCCCCAATCCCCTGC | |
| | TGGGCCTGGACAGCACCTGA | |

In some embodiments a ZZKD Type V Cas protein comprises an amino acid sequence of SEQ ID NO: 43, SEQ ID NO:44, or SEQ ID NO:45. In some embodiments, a ZZKD Type V Cas protein has nickase activity, for example resulting from one or more amino acid substitutions relative to the sequence of SEQ ID NO: 43, SEQ ID NO: 44, or SEQ ID NO:45. In some embodiments, the one or more amino acid substitutions providing nickase activity comprise a D828 substitution, wherein the position of the D828 substitution is defined with respect to the amino acid numbering of SEQ ID NO:44 (corresponding to amino acid 908 of SEQ ID NO: 121). In some embodiments, the one or more amino acid substitutions providing nickase activity comprise an E925 substitution, wherein the position of the E925 substitution is defined with respect to the amino acid numbering of SEQ ID NO:44 (corresponding to amino acid 993 of SEQ ID NO: 121). In some embodiments, the one or more amino acid substitutions providing nickase activity comprise a R1138 substitution, wherein the position of the R1138 substitution is defined with respect to the amino acid numbering of SEQ ID NO:44 (corresponding to amino acid 1226 of SEQ ID NO: 121). In some embodiments, the one or more amino acid substitutions providing nickase activity comprise a D1176 substitution, wherein the position of the D1176 substitution is defined with respect to the amino acid numbering of SEQ ID NO:44 (corresponding to amino acid 1263 of SEQ ID NO:121). In some embodiments, a ZZKD Type V Cas protein is catalytically inactive, for example due to a R1138 substitution in combination with a D828 substitution, a E925 substitution, and/or D1176 substitution.

### 6.2.9. ZXPB Type V Cas Proteins

In one aspect, the disclosure provides ZXPB Type V Cas proteins. ZXPB Type V Cas proteins can be further classified as Type V-A Cas proteins. The ZXPB Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:49. In some embodiments, the ZXPB Type V Cas proteins comprise an amino acid sequence that is at least 95%, at least 96%, at least 97%, at least 98%, or at least 99% identical to SEQ ID NO:49. In some embodiments, a ZXPB Type V Cas protein comprises an amino acid sequence that is identical to SEQ ID NO:49.

Exemplary ZXPB Type V Cas protein sequences and nucleotide sequences encoding exemplary ZXPB Type V Cas proteins are set forth in Table 11.

TABLE 11
ZXPB Type V Cas Sequences

| | | SEQ |
|---|---|---|
| | | ID |
| Name | Sequence | NO. |
| Wildtype | KLEDFTNLYSLSKTLRFELRPIGKTRENIENGGLLRQDEDRAEKYVHIKKLIDEYHKAYI | 49 |
| amino acid | DKQLSGLVLQYADIGKANSLEEYYHSTRKSKDSDKDKIVKIQDNLRKQIVKRLKDSDE | |
| sequence | FKRIDKKELIQSDLAEFIKPAEDRALIAEFKNFTTYFTGFNENRQNMYSDKAISTAIAYR | |
| (without N- | LIHENLPKFIDNIETFDRIAGITELYDQTSSDAEIFRLEHFSETLSQKQIDAYNSVMGRY | |
| terminal | NMLINEYNQTHKQSRLPKFKMLYKQILSDREHPSWLPEQFESDTAVLTAIRECYDDLR | |
| methionine) | IPMANLKTLLEGLGNYDPSGIFLRNDQHLSQISKRLTGDRSSIERSVTEDLLTSRRLNK | |
| | RKSRTTDEEESRKLFKQKGSLSIGYIADTAKIDVERYFAKLGAINTVTEQSENLFAKAE | |
| | NARTTADELLANDYPAGKRLVQSNDDIALLKNLLDALMELQWFVKPLLGTGDEAGKD | |
| | ERFYGEFAQIWEQLDRITPLYNMVRNYVTRKPYSTDKFKLNFESAALLGGWDKNKEP | |
| | DCLSVILRKDEQYYLGIINKNHKKIFENDILPCEGECYDKMVYKLLPGANKMLPKVFFS | |
| | ASRIAEFAPSDEVKRIYNDKTFQKGEKFDLNDCRTLIDFYKASIDKHEEWNKFGFEFS | |
| | DTNNYEDISGFFREVDRQGYKMSFRPVAASYIETLVEEGKLYLFQIYNKDFSAYSKGT | |
| | PNMHTLYWRMLFDERNLSDVVYQLNGGAELFFRRKSLQNGRPTHPANIPIKNKNSR | |
| | NDKKESLFDYDLIKDRRYTVDKFQFHVPITLNFKSDGAGRINERVREYLRSADDVHVI | |
| | GIDRGERNLLYLVVTDMDGNICEQFSLNEICNTDYHSLLDEREHKRMQERQSWQAIE | |
| | GIKELKEGYLSQVVHRIATLMVKYRAIVVLEDLNFGFMRSRQKVEKSVYQKFEHMLID | |
| | KLNYLVDKKANPTTPGGLLKAYQLTDKFESFQKLGKQSGFLFYVPAWNTSKIDPATG | |
| | FVNMLDLGYESIDKAKTLLCKFDSIRYNACKDWFEFALDYDKFGSKATGTRTKWTVC | |
| | TYGQRIDTYRNKDSQWVSRDVDLTNELKSLFSEHGIDIYSNLKDAIVAQNDKEFFANM | |
| | QRILKLTMQMRNSKTGTDTDYIVSPVADANGRFFDSRQADATMPKDADANGAYNIAR | |
| | KGIMLVQQIKQSDDLRTMKFDISNKSWLRFAQHTNQADE | |
| Wildtype | MKLEDFTNLYSLSKTLRFELRPIGKTRENIENGGLLRQDEDRAEKYVHIKKLIDEYHKA | 50 |
| amino acid | YIDKQLSGLVLQYADIGKANSLEEYYHSTRKSKDSDKDKIVKIQDNLRKQIVKRLKDSD | |
| sequence (with | EFKRIDKKELIQSDLAEFIKPAEDRALIAEFKNFTTYFTGFNENRQNMYSDKAISTAIAY | |
| N-terminal | RLIHENLPKFIDNIETFDRIAGITELYDQTSSDAEIFRLEHFSETLSQKQIDAYNSVMGR | |
| methionine) | YNMLINEYNQTHKQSRLPKFKMLYKQILSDREHPSWLPEQFESDTAVLTAIRECYDDL | |
| | RIPMANLKTLLEGLGNYDPSGIFLRNDQHLSQISKRLTGDRSSIERSVTEDLLTSRRLN | |
| | KRKSRTTDEEESRKLFKQKGSLSIGYIADTAKIDVERYFAKLGAINTVTEQSENLFAKA | |
| | ENARTTADELLANDYPAGKRLVQSNDDIALLKNLLDALMELQWFVKPLLGTGDEAGK | |
| | DERFYGEFAQIWEQLDRITPLYNMVRNYVTRKPYSTDKFKLNFESAALLGGWDKNKE | |
| | PDCLSVILRKDEQYYLGIINKNHKKIFENDILPCEGECYDKMVYKLLPGANKMLPKVFF | |
| | SASRIAEFAPSDEVKRIYNDKTFQKGEKFDLNDCRTLIDFYKASIDKHEEWNKFGFEF | |
| | SDTNNYEDISGFFREVDRQGYKMSFRPVAASYIETLVEEGKLYLFQIYNKDFSAYSKG | |
| | TPNMHTLYWRMLFDERNLSDVVYQLNGGAELFFRRKSLQNGRPTHPANIPIKNKNS | |
| | RNDKKESLFDYDLIKDRRYTVDKFQFHVPITLNFKSDGAGRINERVREYLRSADDVHV | |
| | IGIDRGERNLLYLVVTDMDGNICEQFSLNEICNTDYHSLLDEREHKRMQERQSWQAIE | |
| | GIKELKEGYLSQVVHRIATLMVKYRAIVVLEDLNFGFMRSRQKVEKSVYQKFEHMLID | |
| | KLNYLVDKKANPTTPGGLLKAYQLTDKFESFQKLGKQSGFLFYVPAWNTSKIDPATG | |
| | FVNMLDLGYESIDKAKTLLCKFDSIRYNACKDWFEFALDYDKFGSKATGTRTKWTVC | |
| | TYGQRIDTYRNKDSQWVSRDVDLTNELKSLFSEHGIDIYSNLKDAIVAQNDKEFFANM | |
| | QRILKLTMQMRNSKTGTDTDYIVSPVADANGRFFDSRQADATMPKDADANGAYNIAR | |
| | KGIMLVQQIKQSDDLRTMKFDISNKSWLRFAQHTNQADE | |
| Expression | MGKLEDFTNLYSLSKTLRFELRPIGKTRENIENGGLLRQDEDRAEKYVHIKKLIDEYHK | 51 |
| construct (with | AYIDKQLSGLVLQYADIGKANSLEEYYHSTRKSKDSDKDKIVKIQDNLRKQIVKRLKDS | |
| N-terminal | DEFKRIDKKELIQSDLAEFIKPAEDRALIAEFKNFTTYFTGFNENRQNMYSDKAISTAIA | |
| methionine, | YRLIHENLPKFIDNIETFDRIAGITELYDQTSSDAEIFRLEHFSETLSQKQIDAYNSVMG | |
| V5-tag and C- | RYNMLINEYNQTHKQSRLPKFKMLYKQILSDREHPSWLPEQFESDTAVLTAIRECYD | |
| terminal NLS) | DLRIPMANLKTLLEGLGNYDPSGIFLRNDQHLSQISKRLTGDRSSIERSVTEDLLTSRR | |
| aa sequence | LNKRKSRTTDEEESRKLFKQKGSLSIGYIADTAKIDVERYFAKLGAINTVTEQSENLFA | |
| | KAENARTTADELLANDYPAGKRLVQSNDDIALLKNLLDALMELQWFVKPLLGTGDEA | |
| | GKDERFYGEFAQIWEQLDRITPLYNMVRNYVTRKPYSTDKFKLNFESAALLGGWDK | |
| | NKEPDCLSVILRKDEQYYLGIINKNHKKIFENDILPCEGECYDKMVYKLLPGANKMLPK | |
| | VFFSASRIAEFAPSDEVKRIYNDKTFQKGEKFDLNDCRTLIDFYKASIDKHEEWNKFG | |
| | FEFSDTNNYEDISGFFREVDRQGYKMSFRPVAASYIETLVEEGKLYLFQIYNKDFSAY | |
| | SKGTPNMHTLYWRMLFDERNLSDVVYQLNGGAELFFRRKSLQNGRPTHPANIPIKN | |
| | KNSRNDKKESLFDYDLIKDRRYTVDKFQFHVPITLNFKSDGAGRINERVREYLRSADD | |
| | VHVIGIDRGERNLLYLVVTDMDGNICEQFSLNEICNTDYHSLLDEREHKRMQERQSW | |
| | QAIEGIKELKEGYLSQVVHRIATLMVKYRAIVVLEDLNFGFMRSRQKVEKSVYQKFEH | |
| | MLIDKLNYLVDKKANPTTPGGLLKAYQLTDKFESFQKLGKQSGFLFYVPAWNTSKIDP | |
| | ATGFVNMLDLGYESIDKAKTLLCKFDSIRYNACKDWFEFALDYDKFGSKATGTRTKW | |
| | TVCTYGQRIDTYRNKDSQWVSRDVDLTNELKSLFSEHGIDIYSNLKDAIVAQNDKEFF | |
| | ANMQRILKLTMQMRNSKTGTDTDYIVSPVADANGRFFDSRQADATMPKDADANGAY | |
| | NIARKGIMLVQQIKQSDDLRTMKFDISNKSWLRFAQHTNQADESRKRTADGSEFESP | |
| | KKKRKVGSGKPIPNPLLGLDST | |
| Wildtype | ATGAAATTAGAAGATTTTACCAACCTGTATTCGTTATCCAAGACTCTGCGTTTCGA | 52 |
| coding | ACTGCGGCCGATCGGCAAGACACGTGAAAATATCGAAAACGGAGGCCTTTTGAG | |
| sequence (with | GCAGGACGAGGATCGTGCTGAAAAATATGTACACATAAAAAAACTAATCGATGAA | |
| N-terminal | TATCATAAAGCATATATCGATAAACAATTGTCGGGTTTAGTGCTGCAATACGCCGA | |
| methionine | TATCGGTAAAGCCAATTCATTGGAGGAGTATTATCACTCCACAAGAAAGAGCAAA | |
| and stop | GATTCGGACAAGGATAAGATTGTCAAAATCCAGGATAATCTGCGTAAACAAATTG | |
| codon) | TCAAACGGTTGAAAGACTCAGACGAATTCAAGCGTATCGATAAAAAAGAGTTGAT | |
| | TCAATCGGATCTGGCAGAGTTCATAAAACCAGCCGAAGACAGAGCTTTGATTGCC | |
| | GAATTCAAAAACTTCACAACATATTTTACCGGATTCAATGAAAACAGACAGAACAT | |
| | GTATTCGGACAAAGCTATATCTACGGCAATAGCTTATCGTCTGATACATGAGAATC | |
| | TTCCGAAGTTCATAGACAACATAGAGACTTTCGATCGCATCGCCGGTATAACGGA | |
| | ATTGTACGACCAAACCTCCTCCGATGCCGAAATTTTCCGTCTGGAACATTTTTCG | |
| | GAAACACTGAGCCAAAAGCAGATCGATGCCTATAACTCCGTTATGGGCAGATATA | |
| | ACATGCTTATCAATGAGTACAATCAGACGCATAAACAGTCGCGCCTACCTAAATT | |
| | CAAAATGCTGTACAAACAGATTCTTAGCGACCGCGAACACCCCTCGTGGCTGCC | |
| | CGAGCAGTTCGAGTCGGACACGGCTGTATTGACAGCCATTCGCGAATGTTACGA | |
| | TGATCTGCGCATACCTATGGCCAATTTGAAAACGCTTTTAGAGGGGTTGGGCAAC | |
| | TATGACCCGAGTGGAATATTTTTGCGTAATGACCAACATCTCTCTCAGATATCCAA | |
| | ACGATTGACAGGTGATCGGAGTAGCATTGAACGTAGCGTAACAGAAGACCTTCT | |
| | GACATCGAGGAGACTCAACAAGCGAAAAAGCCGCACAACCGACGAGGAGGAATC | |
| | GAGAAAACTGTTCAAGCAAAAGGGTAGTCTGAGTATAGGCTATATAGCTGACACG | |
| | GCCAAAATCGATGTCGAAAGATACTTTGCCAAACTCGGTGCAATAAATACGGTAA | |
| | CGGAGCAGAGCGAGAATCTATTCGCCAAGGCTGAGAATGCCCGCACGACAGCG | |
| | GATGAGCTGCTCGCAAATGATTACCCGGCAGGCAAGAGGCTCGTTCAGTCCAAC | |
| | GACGACATAGCATTGCTGAAAAATCTGCTCGATGCTTTAATGGAGCTGCAATGGT | |
| | TCGTCAAGCCGCTGCTTGGCACGGGGGACGAAGCCGGCAAAGACGAACGTTTC | |
| | TATGGAGAATTTGCACAGATATGGGAGCAGCTGGATCGTATAACGCCTCTCTATA | |
| | ACATGGTGCGCAACTATGTTACCCGCAAGCCGTATTCGACCGACAAATTCAAGCT | |
| | CAACTTTGAGAGCGCAGCGCTTCTCGGCGGCTGGGACAAGAACAAGGAGCCGG | |
| | ACTGTCTGTCGGTAATCTTACGCAAGGATGAGCAATATTATCTCGGCATAATCAAT | |
| | AAGAATCACAAAAAGATATTCGAGAACGATATCTTGCCGTGCGAAGGGGAGTGTT | |
| | ACGACAAAATGGTATATAAACTCCTGCCCGGCGCAAACAAGATGCTGCCGAAAGT | |
| | ATTCTTCTCGGCTTCGCGTATCGCCGAATTTGCACCGAGCGACGAAGTAAAACG | |
| | GATATACAATGATAAGACTTTCCAAAAAGGCGAAAAGTTCGACTTGAACGATTGTC | |
| | GCACACTGATCGACTTCTACAAGGCTTCTATCGACAAACATGAGGAGTGGAACAA | |
| | GTTTGGATTCGAATTCTCGGATACGAACAATTATGAAGACATAAGCGGATTCTTTC | |
| | GCGAGGTCGACAGGCAAGGCTATAAAATGTCATTCCGCCCGGTCGCAGCATCGT | |
| | ATATCGAAACCCTTGTTGAAGAGGGCAAACTCTATCTTTTCCAAATATATAATAAG | |
| | GATTTTTCGGCATATAGCAAAGGTACTCCCAATATGCACACGCTGTATTGGAGGA | |
| | TGCTCTTCGACGAGCGCAATCTATCGGATGTCGTATATCAGCTCAACGGCGGAG | |
| | CAGAGTTGTTCTTCCGAAGAAAGAGTCTTCAAAACGGCCGTCCGACGCATCCGG | |
| | CAAATATTCCTATCAAAAACAAAAACAGTCGGAATGACAAAAAAGAGAGCCTGTTC | |
| | GACTACGATTTGATCAAAGACAGACGCTATACTGTGGACAAATTTCAGTTCCATGT | |
| | CCCGATAACCCTCAATTTCAAGAGCGACGGGGGGGGCAGGATCAACGAGCGTGT | |
| | AAGGGAATATCTCCGCTCGGCGGACGACGTTCACGTCATAGGCATCGACCGCGG | |
| | AGAACGCAATCTGCTGTATCTGGTCGTGACGGATATGGACGGCAATATCTGCGA | |
| | ACAATTCTCGCTCAACGAAATTTGTAATACTGATTATCATTCTTTGTTGGATGAAC | |
| | GCGAACACAAACGTATGCAGGAGAGACAGAGCTGGCAGGCGATAGAGGGCATC | |
| | AAGGAGTTGAAAGAAGGTTATCTGTCTCAGGTCGTACACCGAATCGCGACACTCA | |
| | TGGTTAAATATCGCGCCATTGTCGTACTGGAAGATCTCAACTTCGGCTTCATGCG | |
| | TAGCCGCCAGAAGGTAGAGAAGTCTGTATACCAGAAATTCGAACACATGCTCATA | |
| | GATAAGCTCAATTATCTGGTCGACAAGAAAGCCAATCCGACAACGCCGGGCGGT | |
| | CTGCTAAAAGCCTATCAGTTGACAGACAAATTCGAGAGCTTCCAGAAGCTCGGCA | |
| | AACAGAGCGGATTTCTATTCTACGTTCCGGCATGGAATACATCGAAGATCGATCC | |
| | AGCAACCGGATTCGTCAACATGCTCGATCTCGGATACGAGAGCATCGACAAAGC | |
| | CAAAACACTGCTCTGCAAGTTCGACTCTATACGCTACAATGCGTGCAAAGACTGG | |
| | TTCGAGTTCGCTCTCGATTACGACAAGTTCGGCAGCAAGGCCACCGGTACCCGC | |
| | ACGAAATGGACTGTTTGCACCTACGGACAACGTATCGATACTTATCGCAACAAAG | |
| | ATTCGCAGTGGGTCAGCCGCGACGTCGATTTGACAAATGAGCTGAAATCACTCTT | |
| | CTCCGAACACGGCATAGACATTTACAGCAATCTGAAAGATGCAATAGTCGCACAA | |
| | AACGACAAAGAATTTTTCGCGAACATGCAGCGGATATTGAAACTGACCATGCAAA | |
| | TGCGAAACAGCAAAACGGGTACCGACACAGACTATATCGTCTCGCCCGTCGCCG | |
| | ATGCCAACGGCAGATTCTTCGACAGCAGGCAGGCCGATGCGACCATGCCCAAAG | |
| | ATGCGGATGCGAACGGAGCGTATAATATCGCACGTAAGGGCATTATGCTCGTAC | |
| | AGCAGATCAAGCAGTCCGACGATCTGCGTACAATGAAGTTCGACATAAGCAACAA | |
| | GAGCTGGCTGCGCTTCGCCCAACATACGAACCAGGCGGACGAGTAA | |
| Codon | AAGCTGGAGGACTTCACCAATCTGTACTCTCTGAGCAAGACCCTGCGGTTTGAG | 53 |
| optimized | CTGCGGCCTATCGGCAAGACAAGAGAAAACATCGAAAACGGCGGACTGCTGCGT | |
| coding | CAAGACGAGGACAGAGCCGAAAAGTACGTGCATATTAAAAAGCTGATCGATGAAT | |
| sequence (no | ACCACAAGGCTTATATCGACAAGCAACTGAGTGGCCTGGTCCTGCAATACGCCG | |
| N-terminal | ATATCGGCAAGGCCAATTCTCTGGAGGAGTACTACCACAGCACTAGAAAAAGCAA | |
| methionine, no | GGACTCTGACAAGGATAAGATAGTCAAGATCCAGGACAACCTGCGCAAGCAGAT | |
| stop codon) | CGTCAAGAGATTGAAGGACAGCGATGAGTTTAAGAGGATCGATAAGAAGGAACT | |
| | GATCCAGTCTGACCTGGCAGAGTTCATCAAGCCAGCCGAGGACAGGGCCCTGAT | |
| | AGCCGAGTTCAAGAACTTCACCACCTACTTCACAGGATTCAACGAAAATAGGCAG | |
| | AACATGTACAGCGATAAGGCTATCAGCACCGCCATCGCCTACCGGCTGATCCAC | |
| | GAGAACCTGCCTAAGTTCATCGACAACATCGAAACCTTCGACCGGATCGCGGGC | |
| | ATCACAGAGCTGTATGACCAGACATCCAGCGACGCAGAGATCTTTAGACTGGAG | |
| | CACTTCAGTGAGACACTGAGCCAGAAGCAGATCGATGCCTATAACAGCGTGATG | |
| | GGCCGGTACAACATGCTGATCAACGAATATAACCAGACCCACAAGCAATCTCGG | |
| | CTGCCTAAATTCAAAATGCTGTACAAGCAGATCCTGAGCGACCGGGAGCACCCC | |
| | AGCTGGCTGCCGGAACAGTTCGAGAGCGACACCGCCGTGCTGACCGCCATCAG | |
| | AGAGTGTTACGACGACCTGAGAATCCCTATGGCCAACTTAAAAACCCTGCTTGAG | |
| | GGCCTGGGAAATTACGATCCCTCTGGCATCTTCCTGCGGAACGATCAGCACCTG | |
| | TCTCAGATCAGCAAAAGACTCACCGGAGACAGATCCAGCATCGAACGGAGCGTG | |
| | ACCGAGGACTTATTAACGAGCCGGAGACTGAACAAAAGAAAGAGCAGAACCACC | |
| | GATGAAGAGGAAAGCAGAAAGCTGTTCAAGCAAAAAGGCAGCCTGAGCATCGGC | |
| | TACATCGCCGACACAGCCAAGATCGACGTGGAGAGATACTTCGCCAAGCTGGGA | |
| | GCCATTAATACCGTGACCGAGCAGTCTGAGAACCTCTTCGCTAAGGCCGAGAAC | |
| | GCCAGAACCACTGCTGACGAGCTGCTGGCCAACGACTACCCTGCCGGCAAAAGA | |
| | CTGGTGCAGAGCAACGACGACATCGCTCTGCTGAAGAACCTATTGGACGCCCTG | |
| | ATGGAACTGCAATGGTTCGTGAAGCCCCTGCTGGGCACCGGCGACGAGGCCGG | |
| | CAAAGACGAACGGTTCTATGGCGAGTTCGCTCAGATCTGGGAGCAGCTGGATAG | |
| | AATCACCCCTCTGTACAACATGGTGCGGAATTACGTGACAAGAAAGCCCTACTCC | |
| | ACAGACAAGTTCAAGCTGAACTTCGAATCTGCCGCCCTGCTGGGCGGATGGGAC | |
| | AAGAACAAAGAACCTGACTGCCTGTCCGTGATTCTGAGAAAGGACGAGCAGTAC | |
| | TACCTGGGCATCATCAACAAGAACCACAAGAAGATCTTCGAGAATGACATTCTGC | |
| | CTTGCGAGGGCGAGTGCTACGACAAGATGGTCTACAAGCTGCTGCCTGGCGCTA | |
| | ACAAAATGCTGCCTAAGGTGTTCTTTAGCGCCTCCAGAATCGCTGAGTTCGCCCC | |
| | TTCTGATGAGGTGAAAAGAATTTACAACGATAAGACCTTCCAGAAGGGCGAGAAG | |
| | TTCGATCTGAACGACTGCAGAACCCTCATCGATTTCTACAAGGCTTCTATCGATAA | |
| | GCACGAGGAGTGGAATAAATTTGGCTTCGAGTTTAGCGACACCAACAACTACGA | |
| | GGACATCAGCGGCTTCTTCCGGGAGGTGGACAGACAGGGCTACAAGATGAGCTT | |
| | TAGACCCGTGGCCGCCAGCTACATCGAAACGTTGGTGGAAGAGGGCAAACTGTA | |
| | CCTGTTCCAGATCTACAACAAAGATTTCAGCGCCTACAGCAAGGGCACCCCTAAT | |
| | ATGCACACCCTGTACTGGAGAATGCTGTTTGACGAGCGGAACCTGAGCGACGTG | |
| | GTGTACCAGCTGAACGGCGGAGCTGAACTGTTCTTTAGACGCAAGTCCCTCCAG | |
| | AACGGCCGGCCTACACACCCTGCCAACATCCCTATCAAGAACAAGAACAGCAGA | |
| | AACGATAAAAAGGAATCACTGTTCGACTACGATCTCATCAAGGATCGTAGATACA | |
| | CAGTGGATAAGTTCCAGTTCCACGTGCCAATCACACTGAATTTCAAGAGCGATGG | |
| | CGCTGGCAGAATTAACGAGAGAGTGCGGGAGTACCTGAGATCTGCCGATGACGT | |
| | GCACGTGATCGGCATCGACAGAGGCGAGCGGAACCTGCTGTACCTCGTGGTGA | |
| | CCGATATGGACGGCAACATCTGCGAACAGTTTAGCCTGAACGAAATCTGTAATAC | |
| | CGACTACCACAGCCTGTTGGATGAGAGAGAGCACAAAAGAATGCAGGAAAGACA | |
| | GAGCTGGCAGGCCATCGAGGGAATCAAGGAGCTGAAGGAAGGCTACCTGTCCC | |
| | AAGTGGTCCACAGAATCGCCACCCTGATGGTGAAGTACAGAGCGATCGTGGTGC | |
| | TGGAGGACCTGAACTTCGGCTTCATGCGGAGCAGACAGAAAGTGGAAAAAAGCG | |
| | TGTACCAGAAGTTCGAGCACATGCTGATCGACAAACTGAACTACCTGGTGGACAA | |
| | GAAAGCCAACCCTACCACACCCGGCGGCCTGCTGAAGGCCTACCAGCTGACAG | |
| | ACAAGTTCGAGAGCTTCCAGAAGCTGGGCAAGCAGTCTGGATTCCTGTTTTATGT | |
| | GCCCGCCTGGAACACAAGCAAGATCGACCCTGCTACCGGATTCGTGAACATGCT | |
| | GGATCTGGGCTATGAGAGCATCGACAAGGCCAAAACCCTGCTGTGCAAGTTTGA | |
| | CTCCATCAGATACAACGCCTGCAAGGACTGGTTCGAGTTTGCCCTGGACTACGA | |
| | CAAGTTCGGCAGCAAGGCCACAGGCACACGGACCAAGTGGACAGTGTGCACCT | |
| | ACGGCCAGCGGATCGATACTTATAGAAACAAGGACAGCCAGTGGGTGTCTCGGG | |
| | ACGTGGATCTGACCAATGAGCTGAAGAGCCTGTTTTCTGAACATGGCATCGACAT | |
| | CTACAGCAACCTGAAAGACGCCATCGTGGCCCAAAATGACAAAGAGTTCTTCGC | |
| | CAACATGCAGAGAATCCTGAAGCTGACCATGCAGATGAGAAATTCTAAAACTGGA | |
| | ACAGATACAGACTACATTGTGTCCCCTGTTGCCGATGCTAACGGAAGATTCTTCG | |
| | ACAGCAGACAAGCCGACGCCACCATGCCAAAGGACGCCGACGCCAACGGCGCC | |
| | TACAACATCGCTAGAAAGGGCATCATGCTGGTTCAGCAGATCAAGCAGAGCGAT | |
| | GACCTCCGCACCATGAAATTCGACATCAGCAACAAGAGCTGGCTGAGATTCGCC | |
| | CAGCATACCAACCAGGCCGATGAG | |
| Expression | ATGggcAAGCTGGAGGACTTCACCAATCTGTACTCTCTGAGCAAGACCCTGCGGT | 54 |
| construct (with | TTGAGCTGCGGCCTATCGGCAAGACAAGAGAAAACATCGAAAACGGCGGACTGC | |
| N-terminal | TGCGTCAAGACGAGGACAGAGCCGAAAAGTACGTGCATATTAAAAAGCTGATCG | |
| methionine | ATGAATACCACAAGGCTTATATCGACAAGCAACTGAGTGGCCTGGTCCTGCAATA | |
| and stop | CGCCGATATCGGCAAGGCCAATTCTCTGGAGGAGTACTACCACAGCACTAGAAA | |
| codon, | AAGCAAGGACTCTGACAAGGATAAGATAGTCAAGATCCAGGACAACCTGCGCAA | |
| includes V5- | GCAGATCGTCAAGAGATTGAAGGACAGCGATGAGTTTAAGAGGATCGATAAGAA | |
| tag and C- | GGAACTGATCCAGTCTGACCTGGCAGAGTTCATCAAGCCAGCCGAGGACAGGGC | |
| terminal NLS) | CCTGATAGCCGAGTTCAAGAACTTCACCACCTACTTCACAGGATTCAACGAAAAT | |
| | AGGCAGAACATGTACAGCGATAAGGCTATCAGCACCGCCATCGCCTACCGGCTG | |
| | ATCCACGAGAACCTGCCTAAGTTCATCGACAACATCGAAACCTTCGACCGGATCG | |
| | CGGGCATCACAGAGCTGTATGACCAGACATCCAGCGACGCAGAGATCTTTAGAC | |
| | TGGAGCACTTCAGTGAGACACTGAGCCAGAAGCAGATCGATGCCTATAACAGCG | |
| | TGATGGGCCGGTACAACATGCTGATCAACGAATATAACCAGACCCACAAGCAATC | |
| | TCGGCTGCCTAAATTCAAAATGCTGTACAAGCAGATCCTGAGCGACCGGGAGCA | |
| | CCCCAGCTGGCTGCCGGAACAGTTCGAGAGCGACACCGCCGTGCTGACCGCCA | |
| | TCAGAGAGTGTTACGACGACCTGAGAATCCCTATGGCCAACTTAAAAACCCTGCT | |
| | TGAGGGCCTGGGAAATTACGATCCCTCTGGCATCTTCCTGCGGAACGATCAGCA | |
| | CCTGTCTCAGATCAGCAAAAGACTCACCGGAGACAGATCCAGCATCGAACGGAG | |
| | CGTGACCGAGGACTTATTAACGAGCCGGAGACTGAACAAAAGAAAGAGCAGAAC | |
| | CACCGATGAAGAGGAAAGCAGAAAGCTGTTCAAGCAAAAAGGCAGCCTGAGCAT | |
| | CGGCTACATCGCCGACACAGCCAAGATCGACGTGGAGAGATACTTCGCCAAGCT | |
| | GGGAGCCATTAATACCGTGACCGAGCAGTCTGAGAACCTCTTCGCTAAGGCCGA | |
| | GAACGCCAGAACCACTGCTGACGAGCTGCTGGCCAACGACTACCCTGCCGGCA | |
| | AAAGACTGGTGCAGAGCAACGACGACATCGCTCTGCTGAAGAACCTATTGGACG | |
| | CCCTGATGGAACTGCAATGGTTCGTGAAGCCCCTGCTGGGCACCGGCGACGAG | |
| | GCCGGCAAAGACGAACGGTTCTATGGCGAGTTCGCTCAGATCTGGGAGCAGCTG | |
| | GATAGAATCACCCCTCTGTACAACATGGTGCGGAATTACGTGACAAGAAAGCCCT | |
| | ACTCCACAGACAAGTTCAAGCTGAACTTCGAATCTGCCGCCCTGCTGGGCGGAT | |
| | GGGACAAGAACAAAGAACCTGACTGCCTGTCCGTGATTCTGAGAAAGGACGAGC | |
| | AGTACTACCTGGGCATCATCAACAAGAACCACAAGAAGATCTTCGAGAATGACAT | |
| | TCTGCCTTGCGAGGGCGAGTGCTACGACAAGATGGTCTACAAGCTGCTGCCTGG | |
| | CGCTAACAAAATGCTGCCTAAGGTGTTCTTTAGCGCCTCCAGAATCGCTGAGTTC | |
| | GCCCCTTCTGATGAGGTGAAAAGAATTTACAACGATAAGACCTTCCAGAAGGGCG | |
| | AGAAGTTCGATCTGAACGACTGCAGAACCCTCATCGATTTCTACAAGGCTTCTAT | |
| | CGATAAGCACGAGGAGTGGAATAAATTTGGCTTCGAGTTTAGCGACACCAACAAC | |
| | TACGAGGACATCAGCGGCTTCTTCCGGGAGGTGGACAGACAGGGCTACAAGATG | |
| | AGCTTTAGACCCGTGGCCGCCAGCTACATCGAAACGTTGGTGGAAGAGGGCAAA | |
| | CTGTACCTGTTCCAGATCTACAACAAAGATTTCAGCGCCTACAGCAAGGGCACCC | |
| | CTAATATGCACACCCTGTACTGGAGAATGCTGTTTGACGAGCGGAACCTGAGCG | |
| | ACGTGGTGTACCAGCTGAACGGCGGAGCTGAACTGTTCTTTAGACGCAAGTCCC | |
| | TCCAGAACGGCCGGCCTACACACCCTGCCAACATCCCTATCAAGAACAAGAACA | |
| | GCAGAAACGATAAAAAGGAATCACTGTTCGACTACGATCTCATCAAGGATCGTAG | |
| | ATACACAGTGGATAAGTTCCAGTTCCACGTGCCAATCACACTGAATTTCAAGAGC | |
| | GATGGCGCTGGCAGAATTAACGAGAGAGTGCGGGAGTACCTGAGATCTGCCGAT | |
| | GACGTGCACGTGATCGGCATCGACAGAGGCGAGCGGAACCTGCTGTACCTCGT | |
| | GGTGACCGATATGGACGGCAACATCTGCGAACAGTTTAGCCTGAACGAAATCTG | |
| | TAATACCGACTACCACAGCCTGTTGGATGAGAGAGAGCACAAAAGAATGCAGGA | |
| | AAGACAGAGCTGGCAGGCCATCGAGGGAATCAAGGAGCTGAAGGAAGGCTACC | |
| | TGTCCCAAGTGGTCCACAGAATCGCCACCCTGATGGTGAAGTACAGAGCGATCG | |
| | TGGTGCTGGAGGACCTGAACTTCGGCTTCATGCGGAGCAGACAGAAAGTGGAAA | |
| | AAAGCGTGTACCAGAAGTTCGAGCACATGCTGATCGACAAACTGAACTACCTGGT | |
| | GGACAAGAAAGCCAACCCTACCACACCCGGCGGCCTGCTGAAGGCCTACCAGC | |
| | TGACAGACAAGTTCGAGAGCTTCCAGAAGCTGGGCAAGCAGTCTGGATTCCTGT | |
| | TTTATGTGCCCGCCTGGAACACAAGCAAGATCGACCCTGCTACCGGATTCGTGA | |
| | ACATGCTGGATCTGGGCTATGAGAGCATCGACAAGGCCAAAACCCTGCTGTGCA | |
| | AGTTTGACTCCATCAGATACAACGCCTGCAAGGACTGGTTCGAGTTTGCCCTGGA | |
| | CTACGACAAGTTCGGCAGCAAGGCCACAGGCACACGGACCAAGTGGACAGTGT | |
| | GCACCTACGGCCAGCGGATCGATACTTATAGAAACAAGGACAGCCAGTGGGTGT | |
| | CTCGGGACGTGGATCTGACCAATGAGCTGAAGAGCCTGTTTTCTGAACATGGCA | |
| | TCGACATCTACAGCAACCTGAAAGACGCCATCGTGGCCCAAAATGACAAAGAGTT | |
| | CTTCGCCAACATGCAGAGAATCCTGAAGCTGACCATGCAGATGAGAAATTCTAAA | |
| | ACTGGAACAGATACAGACTACATTGTGTCCCCTGTTGCCGATGCTAACGGAAGAT | |
| | TCTTCGACAGCAGACAAGCCGACGCCACCATGCCAAAGGACGCCGACGCCAAC | |
| | GGCGCCTACAACATCGCTAGAAAGGGCATCATGCTGGTTCAGCAGATCAAGCAG | |
| | AGCGATGACCTCCGCACCATGAAATTCGACATCAGCAACAAGAGCTGGCTGAGA | |
| | TTCGCCCAGCATACCAACCAGGCCGATGAGtctagaAAGCGGACAGCAGACGGCTC | |
| | CGAATTTGAAAGCCCTAAGAAAAAGAGAAAGGTGggatccGGCAAACCTATCCCCA | |
| | ATCCCCTGCTGGGCCTGGACAGCACCTGA | |

In some embodiments a ZXPB Type V Cas protein comprises an amino acid sequence of SEQ ID NO: 49, SEQ ID NO:50, or SEQ ID NO:51. In some embodiments, a ZXPB Type V Cas protein has nickase activity, for example resulting from one or more amino acid substitutions relative to the sequence of SEQ ID NO: 49, SEQ ID NO:50, or SEQ ID NO:51. In some embodiments, the one or more amino acid substitutions providing nickase activity comprise a D821 substitution, wherein the position of the D821 substitution is defined with respect to the amino acid numbering of SEQ ID NO:50 (corresponding to amino acid 908 of SEQ ID NO: 121). In some embodiments, the one or more amino acid substitutions providing nickase activity comprise an E906 substitution, wherein the position of the E906 substitution is defined with respect to the amino acid numbering of SEQ ID NO:50 (corresponding to amino acid 993 of SEQ ID NO: 121). In some embodiments, the one or more amino acid substitutions providing nickase activity comprise a R1116 substitution, wherein the position of the R1116 substitution is defined with respect to the amino acid numbering of SEQ ID NO:50 (corresponding to amino acid 1226 of SEQ ID NO: 121). In some embodiments, the one or more amino acid substitutions providing nickase activity comprise a D1153 substitution, wherein the position of the D1153 substitution is defined with respect to the amino acid numbering of SEQ ID NO:50 (corresponding to amino acid 1263 of SEQ ID NO: 121). In some embodiments, a ZXPB Type V Cas protein is catalytically inactive, for example due to a R1116 substitution in combination with a D821 substitution, a E906 substitution, and/or D1153 substitution.

### 6.2.10. ZPPX Type V Cas Proteins

In one aspect, the disclosure provides ZPPX Type V Cas proteins. ZPPX Type V Cas proteins can be further classified as Type V-A Cas proteins. The ZPPX Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:55. In some embodiments, the ZPPX Type V Cas proteins comprise an amino acid sequence that is at least 95%, at least 96%, at least 97%, at least 98%, or at least 99% identical to SEQ ID NO:55. In some embodiments, a ZPPX Type V Cas protein comprises an amino acid sequence that is identical to SEQ ID NO:55.

Exemplary ZPPX Type V Cas protein sequences and nucleotide sequences encoding exemplary ZPPX Type V Cas proteins are set forth in Table 1J.

TABLE 1J
ZPPX Type V Cas Sequences

| | | SEQ |
|---|---|---|
| | | ID |
| Name | Sequence | NO. |
| Wildtype | MKDLTGQYSLSKTLRFELKPIGKTLEHIEQKGLLTQDEQRAEEYEQMKGIIDRYHKAFI | 55 |
| amino acid | TMCLRNCKIKVNNTDDELDSLEEYSSLLSKSKRDADDENKLEKIKENLRKQIVNAFKS | |
| sequence | GNTYGDLFTKELIKNHLPDFVTDEEEKQVVEHFCNFTTYFTGFHDNRKNMYSDKAKS | |
| (without N- | TAIAYRLIHENFPRFFDNLRSFAKISESEVANRFPEIESAFSLYLNVEHIADMFHVDYFP | |
| terminal | WVLTQEQIDVYNNIIGGKTEEDGTKIQGINEYINLYNQHHPDVKLPFLKPLYKMILSDKV | |
| methionine) | ALSWLPEEFENDEEMLTAINDFYKSVQPVVFGDDENCIRHLLTNIAEYNTDHIYISNDL | |
| | GLTGISQQLFDQYSIFEDVIKDELRRNVKQTPKEKRNPELLEERIKNLFKKEKSFSISYL | |
| | DSLIKDKGEDTIESYYAKLGAFDRDGKQTVNLLTQIEMAYIAAKEVLDGKYDNINQSEE | |
| | ATKYIKDLLDAFKSLQHYIKPLLGSGEEAEKDNVFSSQLLNVWEALDVVTPLYNKVRN | |
| | WLTRKPYSTKKIKLNFENVQLLGGWPNIEAYSCAIFMKDDNTYYLGILDNAYKTLLRD | |
| | FPEPAEEKDTIGLMHYLQGGDMGKNIQNLMVVDGKVRKVNGRKEKSGINVGQNIRLE | |
| | EAKKRYLPTEINRIRKLGTYSVSNPNYNKQDLITIIDYYKPLACEYYASYTFHFKDSSEY | |
| | NSFAEFTDDINQQAYQLGFVPFSQQYLNKLVDEGKLYLFQIWNKDFSDYSKGTPNMH | |
| | TLYWKALFDKANLADVVYKLNGRQAEVFYRKRSLQKENTTVHKALQPIKNKNTQNEK | |
| | STSTFDYDIVKDRRYTVDKFHFHVPITINFKSSGKPNINEHVLDIIRHHGIEHVIGIDRGE | |
| | RHLLYLSLIDLKGRIIKQMTLNEIKQQTGGNYGTNYKELLAAREGDRAEARRNWKKIE | |
| | NIKDLKAGYLSQVVHVIAQMMVEYNAIVVLEDLNMGFMRGRQKIERSVYEQFEHMLID | |
| | KLNFYVDKKKEACAPGGLLHGLQLANKFESFNKLGKQSGCLFYVPAWNTSKIDPVTG | |
| | FVNMLDARYESVESSRRFFSRFDVIRYNEEKNWFEFTFDYNNFHAKLDGTKTQWTL | |
| | CTYGSRIKTFRNPAKLNQWDNEEVVLTDEFKKVFANAGINIHGNLKEAICSLAKREHL | |
| | EPLMHLMKLLLQLRNSKTNSEVDYMLSPVADNGVFYDSRSCNGNLPIDADANGAYNI | |
| | ARKGLWWVLRQIQDSKPGDKLNLALSNKEWLRFVQEKSNFE | |
| Wildtype | MKDLTGQYSLSKTLRFELKPIGKTLEHIEQKGLLTQDEQRAEEYEQMKGIIDRYHKAFI | 56 |
| amino acid | TMCLRNCKIKVNNTDDELDSLEEYSSLLSKSKRDADDENKLEKIKENLRKQIVNAFKS | |
| sequence (with | GNTYGDLFTKELIKNHLPDFVTDEEEKQVVEHFCNFTTYFTGFHDNRKNMYSDKAKS | |
| N-terminal | TAIAYRLIHENFPRFFDNLRSFAKISESEVANRFPEIESAFSLYLNVEHIADMFHVDYFP | |
| methionine) | WVLTQEQIDVYNNIIGGKTEEDGTKIQGINEYINLYNQHHPDVKLPFLKPLYKMILSDKV | |
| | ALSWLPEEFENDEEMLTAINDFYKSVQPVVFGDDENCIRHLLTNIAEYNTDHIYISNDL | |
| | GLTGISQQLFDQYSIFEDVIKDELRRNVKQTPKEKRNPELLEERIKNLFKKEKSFSISYL | |
| | DSLIKDKGEDTIESYYAKLGAFDRDGKQTVNLLTQIEMAYIAAKEVLDGKYDNINQSEE | |
| | ATKYIKDLLDAFKSLQHYIKPLLGSGEEAEKDNVFSSQLLNVWEALDVVTPLYNKVRN | |
| | WLTRKPYSTKKIKLNFENVQLLGGWPNIEAYSCAIFMKDDNTYYLGILDNAYKTLLRD | |
| | FPEPAEEKDTIGLMHYLQGGDMGKNIQNLMVVDGKVRKVNGRKEKSGINVGQNIRLE | |
| | EAKKRYLPTEINRIRKLGTYSVSNPNYNKQDLITIIDYYKPLACEYYASYTFHFKDSSEY | |
| | NSFAEFTDDINQQAYQLGFVPFSQQYLNKLVDEGKLYLFQIWNKDFSDYSKGTPNMH | |
| | TLYWKALFDKANLADVVYKLNGRQAEVFYRKRSLQKENTTVHKALQPIKNKNTQNEK | |
| | STSTFDYDIVKDRRYTVDKFHFHVPITINFKSSGKPNINEHVLDIIRHHGIEHVIGIDRGE | |
| | RHLLYLSLIDLKGRIIKQMTLNEIKQQTGGNYGTNYKELLAAREGDRAEARRNWKKIE | |
| | NIKDLKAGYLSQVVHVIAQMMVEYNAIVVLEDLNMGFMRGRQKIERSVYEQFEHMLID | |
| | KLNFYVDKKKEACAPGGLLHGLQLANKFESFNKLGKQSGCLFYVPAWNTSKIDPVTG | |
| | FVNMLDARYESVESSRRFFSRFDVIRYNEEKNWFEFTFDYNNFHAKLDGTKTQWTL | |
| | CTYGSRIKTFRNPAKLNQWDNEEVVLTDEFKKVFANAGINIHGNLKEAICSLAKREHL | |
| | EPLMHLMKLLLQLRNSKTNSEVDYMLSPVADNGVFYDSRSCNGNLPIDADANGAYNI | |
| | ARKGLWVLRQIQDSKPGDKLNLALSNKEWLRFVQEKSNFE | |
| Expression | MGKDLTGQYSLSKTLRFELKPIGKTLEHIEQKGLLTQDEQRAEEYEQMKGIIDRYHKA | 57 |
| construct (with | FITMCLRNCKIKVNNTDDELDSLEEYSSLLSKSKRDADDENKLEKIKENLRKQIVNAFK | |
| N-terminal | SGNTYGDLFTKELIKNHLPDFVTDEEEKQVVEHFCNFTTYFTGFHDNRKNMYSDKAK | |
| methionine, | STAIAYRLIHENFPRFFDNLRSFAKISESEVANRFPEIESAFSLYLNVEHIADMFHVDYF | |
| V5-tag and C- | PVVLTQEQIDVYNNIIGGKTEEDGTKIQGINEYINLYNQHHPDVKLPFLKPLYKMILSDK | |
| terminal NLS) | VALSWLPEEFENDEEMLTAINDFYKSVQPVVFGDDENCIRHLLTNIAEYNTDHIYISND | |
| aa sequence | LGLTGISQQLFDQYSIFEDVIKDELRRNVKQTPKEKRNPELLEERIKNLFKKEKSFSISY | |
| | LDSLIKDKGEDTIESYYAKLGAFDRDGKQTVNLLTQIEMAYIAAKEVLDGKYDNINQSE | |
| | EATKYIKDLLDAFKSLQHYIKPLLGSGEEAEKDNVFSSQLLNVWEALDVVTPLYNKVR | |
| | NWLTRKPYSTKKIKLNFENVQLLGGWPNIEAYSCAIFMKDDNTYYLGILDNAYKTLLR | |
| | DFPEPAEEKDTIGLMHYLQGGDMGKNIQNLMVVDGKVRKVNGRKEKSGINVGQNIR | |
| | LEEAKKRYLPTEINRIRKLGTYSVSNPNYNKQDLITIIDYYKPLACEYYASYTFHFKDSS | |
| | EYNSFAEFTDDINQQAYQLGFVPFSQQYLNKLVDEGKLYLFQIWNKDFSDYSKGTPN | |
| | MHTLYWKALFDKANLADVVYKLNGRQAEVFYRKRSLQKENTTVHKALQPIKNKNTQ | |
| | NEKSTSTFDYDIVKDRRYTVDKFHFHVPITINFKSSGKPNINEHVLDIIRHHGIEHVIGID | |
| | RGERHLLYLSLIDLKGRIIKQMTLNEIKQQTGGNYGTNYKELLAAREGDRAEARRNWK | |
| | KIENIKDLKAGYLSQVVHVIAQMMVEYNAIVVLEDLNMGFMRGRQKIERSVYEQFEH | |
| | MLIDKLNFYVDKKKEACAPGGLLHGLQLANKFESFNKLGKQSGCLFYVPAWNTSKID | |
| | PVTGFVNMLDARYESVESSRRFFSRFDVIRYNEEKNWFEFTFDYNNFHAKLDGTKT | |
| | QWTLCTYGSRIKTFRNPAKLNQWDNEEVVLTDEFKKVFANAGINIHGNLKEAICSLAK | |
| | REHLEPLMHLMKLLLQLRNSKTNSEVDYMLSPVADNGVFYDSRSCNGNLPIDADAN | |
| | GAYNIARKGLWVLRQIQDSKPGDKLNLALSNKEWLRFVQEKSNFESRKRTADGSEF | |
| | ESPKKKRKVGSGKPIPNPLLGLDST | |
| Wildtype | ATGAAAGACCTGACAGGGCAATATAGCCTGTCGAAAACTTTACGATTTGAGTTAA | 58 |
| coding | AACCTATCGGTAAAACTCTTGAGCACATTGAGCAAAAAGGACTCTTGACACAGGA | |
| sequence (with | CGAACAAAGAGCAGAAGAGTACGAGCAAATGAAAGGTATCATCGACCGATATCA | |
| N-terminal | CAAGGCATTTATTACCATGTGTTTGAGAAACTGCAAAATCAAGGTAAATAATACAG | |
| methionine | ACGACGAATTAGACTCATTAGAAGAATACTCCTCATTACTTTCCAAAAGTAAAAGA | |
| and stop | GATGCTGATGATGAGAACAAATTGGAAAAGATTAAGGAAAATCTTCGCAAGCAAA | |
| codon) | TCGTCAATGCTTTCAAAAGCGGCAACACTTATGGCGACTTGTTCACAAAGGAACT | |
| | GATTAAGAATCATCTGCCCGACTTCGTCACAGACGAGGAAGAAAAGCAAGTGGT | |
| | GGAGCATTTCTGCAATTTTACCACATATTTTACGGGTTTCCACGACAACCGCAAAA | |
| | ACATGTACTCAGATAAGGCTAAATCCACGGCAATAGCCTATCGCCTGATACATGA | |
| | GAATTTCCCTCGGTTTTTTGACAATCTTCGCTCTTTTGCAAAGATTTCAGAAAGCG | |
| | AGGTGGCAAATCGGTTCCCTGAGATAGAATCTGCTTTCTCTCTGTATCTCAACGT | |
| | GGAACACATCGCCGACATGTTCCACGTTGACTATTTCCCAGTTGTTCTTACCCAA | |
| | GAACAAATTGATGTGTATAATAATATTATTGGAGGCAAGACGGAAGAAGATGGGA | |
| | CAAAAATACAGGGCATCAATGAATACATCAACCTTTATAACCAACATCACCCAGAT | |
| | GTAAAGTTGCCGTTCTTGAAACCTCTATACAAGATGATTCTTAGCGACAAGGTTG | |
| | CGCTTTCATGGTTGCCGGAGGAGTTTGAGAATGATGAAGAGATGTTGACGGCCA | |
| | TAAATGATTTTTACAAGTCAGTTCAGCCTGTCGTTTTCGGGGATGACGAGAATTGT | |
| | ATCCGTCATCTTCTGACGAATATTGCCGAATACAATACGGATCACATATACATTTC | |
| | AAACGATTTAGGATTGACTGGAATATCCCAGCAATTGTTCGACCAATACAGCATCT | |
| | TTGAAGACGTCATTAAAGATGAGTTGAGGCGTAATGTCAAACAGACGCCCAAAGA | |
| | GAAACGCAATCCTGAATTGTTGGAAGAAAGAATAAAGAACTTGTTCAAGAAAGAG | |
| | AAGAGTTTCTCCATCTCTTACCTGGACTCTCTCATTAAGGATAAGGGTGAGGATA | |
| | CGATCGAGTCTTATTATGCCAAACTTGGTGCGTTTGACAGAGACGGTAAGCAAAC | |
| | AGTGAATTTGCTCACGCAAATTGAAATGGCATACATAGCGGCAAAGGAGGTGCTT | |
| | GATGGTAAGTATGACAACATTAACCAGTCTGAAGAAGCAACGAAATATATTAAAGA | |
| | TCTTCTTGATGCGTTCAAGTCTTTGCAACACTACATCAAACCGCTGTTAGGTAGTG | |
| | GCGAAGAAGCAGAAAAGGATAATGTGTTTAGTTCGCAACTGCTCAATGTTTGGGA | |
| | GGCGTTAGACGTTGTGACTCCTCTTTATAACAAAGTTCGCAACTGGCTCACACGC | |
| | AAGCCTTACTCAACAAAAAAGATAAAGCTGAACTTTGAGAATGTCCAACTGCTTG | |
| | GCGGCTGGCCAAATATAGAAGCGTATTCATGTGCTATTTTTATGAAGGATGATAAT | |
| | ACTTACTATCTTGGAATACTGGACAATGCATATAAAACTTTATTAAGAGATTTTCCA | |
| | GAGCCTGCCGAAGAGAAGGATACTATTGGGCTAATGCATTACCTCCAAGGAGGC | |
| | GATATGGGAAAAAATATTCAGAATTTGATGGTGGTAGATGGAAAGGTTCGGAAAG | |
| | TTAATGGGCGCAAAGAGAAGTCAGGAATTAATGTTGGGCAGAATATTCGATTAGA | |
| | AGAAGCAAAAAAGAGATACCTGCCAACAGAAATCAATAGAATAAGGAAGTTGGGA | |
| | ACGTATTCTGTTTCAAATCCAAATTATAACAAACAAGATTTGATAACCATAATCGAT | |
| | TATTACAAGCCACTGGCTTGTGAATACTATGCTTCCTATACATTCCATTTCAAGGA | |
| | TTCTTCCGAGTATAATTCGTTCGCGGAGTTTACAGACGATATCAATCAGCAAGCG | |
| | TATCAACTTGGGTTTGTACCTTTTTCTCAACAATACTTAAACAAACTTGTAGACGAA | |
| | GGCAAACTCTACCTTTTCCAAATATGGAATAAAGATTTCTCTGATTATAGTAAAGG | |
| | CACTCCCAATATGCATACCCTTTATTGGAAGGCGCTCTTTGATAAAGCAAATCTTG | |
| | CCGATGTTGTCTACAAACTTAATGGTCGTCAGGCAGAGGTGTTCTATCGGAAAAG | |
| | AAGCCTCCAAAAAGAGAATACGACTGTGCACAAAGCATTGCAGCCTATAAAGAAT | |
| | AAAAACACGCAGAATGAGAAAAGCACCAGTACGTTTGACTATGACATCGTAAAAG | |
| | ATCGTCGTTATACAGTTGATAAATTCCATTTCCATGTGCCCATTACTATTAACTTTA | |
| | AGTCATCTGGAAAACCTAATATCAATGAACACGTTTTAGATATTATCCGTCACCAT | |
| | GGCATTGAGCATGTCATCGGAATCGACCGTGGCGAGCGCCATCTATTATATCTTT | |
| | CTCTTATAGATCTCAAGGGAAGAATAATCAAGCAAATGACGCTTAATGAGATAAAG | |
| | CAGCAAACAGGCGGTAACTATGGCACAAATTATAAAGAACTCTTGGCCGCAAGAG | |
| | AAGGCGATCGTGCGGAAGCGCGTCGTAACTGGAAAAAGATAGAGAATATTAAAG | |
| | ACCTTAAAGCTGGCTATCTCAGTCAGGTTGTACATGTGATAGCCCAAATGATGGT | |
| | GGAATACAATGCCATCGTTGTGCTCGAAGACCTCAATATGGGCTTTATGCGTGGG | |
| | CGGCAGAAAATCGAGCGGAGCGTATACGAGCAGTTCGAACACATGCTGATAGAT | |
| | AAGTTGAACTTCTATGTTGATAAGAAAAAGGAAGCATGTGCCCCCGGAGGTCTGC | |
| | TTCATGGTCTCCAATTAGCCAATAAATTTGAGAGCTTCAATAAGCTTGGGAAACAG | |
| | AGCGGTTGCCTTTTTTATGTACCGGCATGGAATACCAGCAAAATAGATCCTGTCA | |
| | CAGGGTTTGTCAATATGCTTGATGCACGCTATGAAAGTGTAGAAAGTTCGCGCCG | |
| | CTTCTTCTCTCGTTTCGATGTTATTCGTTACAATGAGGAAAAGAATTGGTTTGAAT | |
| | TTACTTTTGATTATAATAACTTCCATGCAAAGTTGGACGGGACAAAAACCCAATGG | |
| | ACGCTTTGCACATACGGCAGTCGCATCAAAACATTCCGCAACCCCGCAAAACTCA | |
| | ATCAATGGGATAATGAAGAGGTGGTTCTTACCGATGAATTTAAGAAGGTATTTGC | |
| | CAATGCTGGTATCAATATTCATGGGAATTTGAAAGAGGCCATTTGCTCTCTTGCTA | |
| | AACGGGAGCATTTAGAACCGTTGATGCATTTGATGAAACTGCTTTTACAGTTGCG | |
| | CAACAGCAAGACCAACTCAGAGGTCGACTATATGCTTTCTCCTGTGGCAGATAAT | |
| | GGCGTGTTTTACGACAGCCGTTCTTGCAATGGCAATTTGCCTATAGATGCCGATG | |
| | CCAATGGGGCATACAACATTGCCCGGAAAGGATTATGGGTTTTGCGCCAAATTCA | |
| | GGACTCTAAGCCTGGCGACAAACTGAATTTGGCTTTGTCGAACAAGGAATGGTTG | |
| | CGATTTGTTCAAGAAAAGAGCAACTTTGAATAA | |
| Codon | AAGGATCTGACAGGCCAGTACAGCCTCTCTAAGACCCTCAGATTTGAACTGAAGC | 59 |
| optimized | CTATCGGCAAGACCCTGGAGCACATCGAGCAAAAGGGCCTGCTGACCCAGGAC | |
| coding | GAGCAGAGAGCCGAGGAATACGAGCAGATGAAGGGAATTATTGACAGATACCAC | |
| sequence (no | AAGGCCTTCATCACTATGTGCCTGAGAAATTGCAAGATCAAGGTGAACAACACCG | |
| N-terminal | ACGATGAGCTGGACAGCCTGGAAGAGTACAGCAGCCTGCTGTCAAAGTCTAAGC | |
| methionine, no | GGGACGCCGACGACGAGAACAAACTGGAGAAGATCAAGGAAAACCTGAGAAAG | |
| stop codon) | CAGATCGTCAATGCCTTCAAGAGCGGAAACACCTACGGCGATCTGTTCACCAAG | |
| | GAGCTGATCAAGAACCACCTCCCCGATTTTGTGACCGACGAGGAAGAAAAGCAG | |
| | GTGGTGGAACACTTCTGCAACTTCACCACCTACTTCACCGGCTTTCACGACAACC | |
| | GCAAGAACATGTACAGCGACAAGGCCAAGAGCACAGCCATCGCCTACAGACTGA | |
| | TCCACGAGAACTTTCCAAGATTTTTCGATAATCTGCGGAGCTTTGCCAAGATCTC | |
| | CGAATCTGAAGTGGCCAACAGATTCCCAGAAATCGAGAGCGCCTTTAGCCTGTA | |
| | CCTGAATGTGGAACATATCGCCGATATGTTCCACGTGGACTACTTCCCAGTGGTG | |
| | CTGACCCAGGAGCAGATTGACGTGTACAACAACATCATCGGAGGCAAGACCGAG | |
| | GAAGATGGCACAAAGATTCAGGGCATCAACGAGTATATCAACCTGTACAACCAAC | |
| | ACCATCCTGACGTCAAACTGCCCTTCCTGAAGCCTCTGTATAAGATGATCCTGAG | |
| | CGACAAGGTGGCCCTGAGCTGGCTGCCTGAAGAGTTCGAGAACGACGAGGAAA | |
| | TGCTGACCGCCATCAATGATTTCTACAAGTCTGTGCAGCCTGTGGTGTTCGGCGA | |
| | TGACGAGAACTGTATCAGACACCTGCTGACAAACATCGCCGAGTACAACACCGAT | |
| | CACATTTACATCAGCAATGACCTGGGACTGACTGGCATCTCTCAGCAGCTGTTCG | |
| | ACCAGTACTCTATCTTCGAAGATGTGATCAAGGACGAGCTACGGCGGAACGTGA | |
| | AGCAAACACCTAAGGAGAAGCGGAACCCCGAACTGCTGGAAGAGAGAATCAAGA | |
| | ACCTGTTCAAGAAAGAAAAGAGCTTCTCCATCAGCTACCTGGATAGCCTGATCAA | |
| | GGACAAAGGAGAAGATACCATCGAGAGCTACTACGCCAAGCTGGGCGCCTTCGA | |
| | CAGAGATGGCAAGCAGACAGTGAACCTGCTCACCCAGATCGAGATGGCCTACAT | |
| | CGCCGCTAAGGAAGTGCTGGATGGCAAGTACGACAACATCAACCAGAGCGAGGA | |
| | AGCTACAAAGTACATCAAGGATCTGCTTGACGCCTTCAAGAGCCTGCAGCACTAC | |
| | ATCAAGCCCCTGCTGGGCAGCGGCGAGGAGGCCGAAAAAGACAACGTGTTCAG | |
| | CAGCCAGCTCCTGAACGTGTGGGAGGCTCTGGACGTGGTGACGCCTCTGTACAA | |
| | CAAGGTCAGAAATTGGCTGACAAGAAAGCCCTACAGTACCAAGAAAATCAAACTG | |
| | AACTTCGAGAATGTTCAACTGCTGGGCGGATGGCCTAACATCGAGGCCTATAGC | |
| | TGCGCCATTTTTATGAAAGACGACAACACCTACTACTTAGGCATCCTGGACAACG | |
| | CCTATAAAACACTACTTCGGGACTTTCCTGAACCTGCTGAAGAAAAGGACACAAT | |
| | CGGCCTGATGCACTACCTGCAAGGAGGCGACATGGGCAAGAACATCCAGAACCT | |
| | GATGGTCGTCGACGGGAAGGTGCGGAAGGTGAACGGCCGTAAGGAAAAGTCCG | |
| | GCATCAACGTGGGCCAGAATATCCGGCTGGAGGAGGCCAAGAAGAGATACCTG | |
| | CCTACAGAGATCAACAGAATCAGAAAGCTGGGCACCTACTCTGTGAGCAACCCTA | |
| | ATTATAACAAGCAGGATCTGATTACAATCATCGACTACTACAAGCCACTGGCCTG | |
| | CGAGTACTACGCCTCTTATACATTCCACTTCAAGGACAGCAGCGAGTACAACAGC | |
| | TTCGCCGAGTTCACCGATGATATCAACCAGCAGGCCTACCAGTTGGGCTTCGTG | |
| | CCTTTCTCCCAGCAATACCTCAACAAACTGGTGGACGAGGGCAAGCTGTACCTGT | |
| | TCCAGATCTGGAATAAGGACTTCTCTGACTACTCTAAGGGCACCCCCAACATGCA | |
| | CACCCTGTACTGGAAGGCCCTGTTTGACAAGGCCAATCTGGCTGATGTGGTTTAC | |
| | AAGCTGAACGGCAGACAGGCCGAGGTGTTTTACAGAAAGAGAAGCCTGCAGAAA | |
| | GAGAACACAACCGTGCACAAGGCTCTGCAGCCCATCAAGAATAAGAACACACAG | |
| | AACGAGAAATCTACCAGCACATTCGATTACGATATCGTGAAGGACAGAAGATACA | |
| | CCGTGGACAAGTTCCATTTCCACGTTCCTATCACCATCAACTTCAAGTCCAGCGG | |
| | CAAGCCTAACATCAACGAGCATGTGCTGGATATCATCAGACACCACGGCATCGA | |
| | GCACGTGATCGGCATCGACCGCGGCGAAAGGCACCTGCTGTACCTGTCCCTGAT | |
| | CGACCTGAAAGGACGGATCATAAAGCAGATGACCCTTAACGAGATCAAACAACA | |
| | GACCGGCGGCAACTACGGCACAAACTACAAAGAGCTGCTGGCCGCCAGAGAAG | |
| | GCGACAGAGCCGAGGCTAGAAGAAACTGGAAGAAAATCGAGAACATCAAGGACC | |
| | TGAAGGCCGGCTACCTGAGCCAGGTGGTGCACGTGATTGCTCAGATGATGGTGG | |
| | AATACAACGCCATTGTAGTGCTGGAGGACCTGAACATGGGCTTCATGAGAGGCA | |
| | GACAGAAGATCGAGAGAAGCGTGTACGAGCAGTTCGAGCACATGCTGATTGACA | |
| | AGCTGAACTTCTACGTGGACAAAAAGAAGGAAGCATGCGCCCCTGGCGGACTTC | |
| | TGCACGGCCTGCAGCTGGCCAACAAATTCGAGTCTTTCAACAAACTGGGCAAGC | |
| | AATCCGGCTGTCTGTTCTACGTGCCCGCCTGGAACACCAGCAAGATCGATCCTG | |
| | TGACCGGATTCGTGAACATGCTGGACGCCCGGTACGAGAGCGTGGAGAGCTCC | |
| | CGGCGGTTCTTCTCCAGATTTGACGTGATCAGATACAACGAGGAGAAGAACTGG | |
| | TTCGAGTTCACCTTTGATTATAACAACTTCCACGCCAAACTGGATGGCACCAAGA | |
| | CCCAGTGGACACTGTGCACCTACGGCAGCAGAATCAAGACCTTTAGAAATCCTG | |
| | CTAAGCTGAATCAGTGGGACAATGAAGAGGTGGTTCTGACCGACGAATTTAAGAA | |
| | GGTGTTCGCCAACGCCGGAATCAATATCCACGGCAACCTGAAGGAAGCTATCTG | |
| | CAGCCTGGCCAAAAGAGAGCACCTGGAACCTCTGATGCACCTGATGAAACTGCT | |
| | GCTGCAACTTCGGAATAGCAAAACCAACAGCGAGGTCGACTACATGCTGTCTCC | |
| | AGTGGCCGATAATGGAGTGTTCTACGACAGCAGAAGCTGTAACGGTAACCTGCC | |
| | TATCGACGCCGACGCCAACGGAGCCTACAATATCGCTAGAAAAGGTCTGTGGGT | |
| | CCTCAGGCAAATCCAGGATAGCAAGCCCGGCGACAAGCTGAACCTGGCTCTGAG | |
| | CAACAAGGAATGGCTGCGATTTGTACAGGAGAAAAGCAATTTCGAG | |
| Expression | ATGggcAAGGATCTGACAGGCCAGTACAGCCTCTCTAAGACCCTCAGATTTGAACT | 60 |
| construct (with | GAAGCCTATCGGCAAGACCCTGGAGCACATCGAGCAAAAGGGCCTGCTGACCCA | |
| N-terminal | GGACGAGCAGAGAGCCGAGGAATACGAGCAGATGAAGGGAATTATTGACAGATA | |
| methionine | CCACAAGGCCTTCATCACTATGTGCCTGAGAAATTGCAAGATCAAGGTGAACAAC | |
| and stop | ACCGACGATGAGCTGGACAGCCTGGAAGAGTACAGCAGCCTGCTGTCAAAGTCT | |
| codon, | AAGCGGGACGCCGACGACGAGAACAAACTGGAGAAGATCAAGGAAAACCTGAG | |
| includes V5- | AAAGCAGATCGTCAATGCCTTCAAGAGCGGAAACACCTACGGCGATCTGTTCAC | |
| tag and C- | CAAGGAGCTGATCAAGAACCACCTCCCCGATTTTGTGACCGACGAGGAAGAAAA | |
| terminal NLS) | GCAGGTGGTGGAACACTTCTGCAACTTCACCACCTACTTCACCGGCTTTCACGAC | |
| | AACCGCAAGAACATGTACAGCGACAAGGCCAAGAGCACAGCCATCGCCTACAGA | |
| | CTGATCCACGAGAACTTTCCAAGATTTTTCGATAATCTGCGGAGCTTTGCCAAGA | |
| | TCTCCGAATCTGAAGTGGCCAACAGATTCCCAGAAATCGAGAGCGCCTTTAGCCT | |
| | GTACCTGAATGTGGAACATATCGCCGATATGTTCCACGTGGACTACTTCCCAGTG | |
| | GTGCTGACCCAGGAGCAGATTGACGTGTACAACAACATCATCGGAGGCAAGACC | |
| | GAGGAAGATGGCACAAAGATTCAGGGCATCAACGAGTATATCAACCTGTACAACC | |
| | AACACCATCCTGACGTCAAACTGCCCTTCCTGAAGCCTCTGTATAAGATGATCCT | |
| | GAGCGACAAGGTGGCCCTGAGCTGGCTGCCTGAAGAGTTCGAGAACGACGAGG | |
| | AAATGCTGACCGCCATCAATGATTTCTACAAGTCTGTGCAGCCTGTGGTGTTCGG | |
| | CGATGACGAGAACTGTATCAGACACCTGCTGACAAACATCGCCGAGTACAACAC | |
| | CGATCACATTTACATCAGCAATGACCTGGGACTGACTGGCATCTCTCAGCAGCTG | |
| | TTCGACCAGTACTCTATCTTCGAAGATGTGATCAAGGACGAGCTACGGCGGAAC | |
| | GTGAAGCAAACACCTAAGGAGAAGCGGAACCCCGAACTGCTGGAAGAGAGAATC | |
| | AAGAACCTGTTCAAGAAAGAAAAGAGCTTCTCCATCAGCTACCTGGATAGCCTGA | |
| | TCAAGGACAAAGGAGAAGATACCATCGAGAGCTACTACGCCAAGCTGGGCGCCT | |
| | TCGACAGAGATGGCAAGCAGACAGTGAACCTGCTCACCCAGATCGAGATGGCCT | |
| | ACATCGCCGCTAAGGAAGTGCTGGATGGCAAGTACGACAACATCAACCAGAGCG | |
| | AGGAAGCTACAAAGTACATCAAGGATCTGCTTGACGCCTTCAAGAGCCTGCAGC | |
| | ACTACATCAAGCCCCTGCTGGGCAGCGGCGAGGAGGCCGAAAAAGACAACGTG | |
| | TTCAGCAGCCAGCTCCTGAACGTGTGGGAGGCTCTGGACGTGGTGACGCCTCTG | |
| | TACAACAAGGTCAGAAATTGGCTGACAAGAAAGCCCTACAGTACCAAGAAAATCA | |
| | AACTGAACTTCGAGAATGTTCAACTGCTGGGCGGATGGCCTAACATCGAGGCCT | |
| | ATAGCTGCGCCATTTTTATGAAAGACGACAACACCTACTACTTAGGCATCCTGGA | |
| | CAACGCCTATAAAACACTACTTCGGGACTTTCCTGAACCTGCTGAAGAAAAGGAC | |
| | ACAATCGGCCTGATGCACTACCTGCAAGGAGGCGACATGGGCAAGAACATCCAG | |
| | AACCTGATGGTCGTCGACGGGAAGGTGCGGAAGGTGAACGGCCGTAAGGAAAA | |
| | GTCCGGCATCAACGTGGGCCAGAATATCCGGCTGGAGGAGGCCAAGAAGAGAT | |
| | ACCTGCCTACAGAGATCAACAGAATCAGAAAGCTGGGCACCTACTCTGTGAGCA | |
| | ACCCTAATTATAACAAGCAGGATCTGATTACAATCATCGACTACTACAAGCCACTG | |
| | GCCTGCGAGTACTACGCCTCTTATACATTCCACTTCAAGGACAGCAGCGAGTACA | |
| | ACAGCTTCGCCGAGTTCACCGATGATATCAACCAGCAGGCCTACCAGTTGGGCT | |
| | TCGTGCCTTTCTCCCAGCAATACCTCAACAAACTGGTGGACGAGGGCAAGCTGT | |
| | ACCTGTTCCAGATCTGGAATAAGGACTTCTCTGACTACTCTAAGGGCACCCCCAA | |
| | CATGCACACCCTGTACTGGAAGGCCCTGTTTGACAAGGCCAATCTGGCTGATGT | |
| | GGTTTACAAGCTGAACGGCAGACAGGCCGAGGTGTTTTACAGAAAGAGAAGCCT | |
| | GCAGAAAGAGAACACAACCGTGCACAAGGCTCTGCAGCCCATCAAGAATAAGAA | |
| | CACACAGAACGAGAAATCTACCAGCACATTCGATTACGATATCGTGAAGGACAGA | |
| | AGATACACCGTGGACAAGTTCCATTTCCACGTTCCTATCACCATCAACTTCAAGTC | |
| | CAGCGGCAAGCCTAACATCAACGAGCATGTGCTGGATATCATCAGACACCACGG | |
| | CATCGAGCACGTGATCGGCATCGACCGCGGCGAAAGGCACCTGCTGTACCTGTC | |
| | CCTGATCGACCTGAAAGGACGGATCATAAAGCAGATGACCCTTAACGAGATCAAA | |
| | CAACAGACCGGCGGCAACTACGGCACAAACTACAAAGAGCTGCTGGCCGCCAG | |
| | AGAAGGCGACAGAGCCGAGGCTAGAAGAAACTGGAAGAAAATCGAGAACATCAA | |
| | GGACCTGAAGGCCGGCTACCTGAGCCAGGTGGTGCACGTGATTGCTCAGATGAT | |
| | GGTGGAATACAACGCCATTGTAGTGCTGGAGGACCTGAACATGGGCTTCATGAG | |
| | AGGCAGACAGAAGATCGAGAGAAGCGTGTACGAGCAGTTCGAGCACATGCTGAT | |
| | TGACAAGCTGAACTTCTACGTGGACAAAAAGAAGGAAGCATGCGCCCCTGGCGG | |
| | ACTTCTGCACGGCCTGCAGCTGGCCAACAAATTCGAGTCTTTCAACAAACTGGGC | |
| | AAGCAATCCGGCTGTCTGTTCTACGTGCCCGCCTGGAACACCAGCAAGATCGAT | |
| | CCTGTGACCGGATTCGTGAACATGCTGGACGCCCGGTACGAGAGCGTGGAGAG | |
| | CTCCCGGCGGTTCTTCTCCAGATTTGACGTGATCAGATACAACGAGGAGAAGAA | |
| | CTGGTTCGAGTTCACCTTTGATTATAACAACTTCCACGCCAAACTGGATGGCACC | |
| | AAGACCCAGTGGACACTGTGCACCTACGGCAGCAGAATCAAGACCTTTAGAAAT | |
| | CCTGCTAAGCTGAATCAGTGGGACAATGAAGAGGTGGTTCTGACCGACGAATTTA | |
| | AGAAGGTGTTCGCCAACGCCGGAATCAATATCCACGGCAACCTGAAGGAAGCTA | |
| | TCTGCAGCCTGGCCAAAAGAGAGCACCTGGAACCTCTGATGCACCTGATGAAAC | |
| | TGCTGCTGCAACTTCGGAATAGCAAAACCAACAGCGAGGTCGACTACATGCTGT | |
| | CTCCAGTGGCCGATAATGGAGTGTTCTACGACAGCAGAAGCTGTAACGGTAACC | |
| | TGCCTATCGACGCCGACGCCAACGGAGCCTACAATATCGCTAGAAAAGGTCTGT | |
| | GGGTCCTCAGGCAAATCCAGGATAGCAAGCCCGGCGACAAGCTGAACCTGGCT | |
| | CTGAGCAACAAGGAATGGCTGCGATTTGTACAGGAGAAAAGCAATTTCGAGtctaga | |
| | AAGCGGACAGCAGACGGCTCCGAATTTGAAAGCCCTAAGAAAAAGAGAAAGGTG | |
| | ggatccGGCAAACCTATCCCCAATCCCCTGCTGGGCCTGGACAGCACCTGA | |

In some embodiments a ZPPX Type V Cas protein comprises an amino acid sequence of SEQ ID NO: 55, SEQ ID NO:56, or SEQ ID NO:57. In some embodiments, a ZPPX Type V Cas protein has nickase activity, for example resulting from one or more amino acid substitutions relative to the sequence of SEQ ID NO:55, SEQ ID NO:56, or SEQ ID NO:57. In some embodiments, the one or more amino acid substitutions providing nickase activity comprise a D877 substitution, wherein the position of the D877 substitution is defined with respect to the amino acid numbering of SEQ ID NO:56 (corresponding to amino acid 908 of SEQ ID NO: 121). In some embodiments, the one or more amino acid substitutions providing nickase activity comprise an E969 substitution, wherein the position of the E969 substitution is defined with respect to the amino acid numbering of SEQ ID NO:56 (corresponding to amino acid 993 of SEQ ID NO: 121). In some embodiments, the one or more amino acid substitutions providing nickase activity comprise a R1181 substitution, wherein the position of the R1181 substitution is defined with respect to the amino acid numbering of SEQ ID NO:56 (corresponding to amino acid 1226 of SEQ ID NO: 121). In some embodiments, the one or more amino acid substitutions providing nickase activity comprise a D1217 substitution, wherein the position of the D1217 substitution is defined with respect to the amino acid numbering of SEQ ID NO:56 (corresponding to amino acid 1263 of SEQ ID NO: 121). In some embodiments, a ZPPX Type V Cas protein is catalytically inactive, for example due to a R1181 substitution in combination with a D877 substitution, a E969 substitution, and/or D1217 substitution.

### 6.2.11. ZXHQ Type V Cas Proteins

In one aspect, the disclosure provides ZXHQ Type V Cas proteins. ZXHQ Type V Cas proteins can be further classified as Type V-A Cas proteins. The ZXHQ Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:61. In some embodiments, the ZXHQ Type V Cas proteins comprise an amino acid sequence that is at least 95%, at least 96%, at least 97%, at least 98%, or at least 99% identical to SEQ ID NO:61. In some embodiments, a ZXHQ Type V Cas protein comprises an amino acid sequence that is identical to SEQ ID NO:61.

Exemplary ZXHQ Type V Cas protein sequences and nucleotide sequences encoding exemplary ZXHQ Type V Cas proteins are set forth in Table 1K.

TABLE 1K
ZXHQ Type V Cas Sequences

| | | SEQ ID |
|---|---|---|
| Name | Sequence | NO. |
| Wildtype | TNTSIFKTFTNQYSLSKTLRFELRPHPMTSGLDDIISLDTGIKKLYENEMKPLFDEL | 61 |
| amino acid | HFEFISQSLVQVSFPSEKLEVLLNKYRSLKDQKAKNIEKELEGPLQELRTIITDTFES | |
| sequence | TGNNWKKEWLQQGFKIKSSGYKVLTEEGILEVLSVRKKDKADAINKFKGFFTYFS | |
| (without N- | GFNMNRENYYSSEDKKTAVAYRVINENLIRYMDNILLLQNVLAKAPEFKKFEDILSL | |
| terminal | TTFGKYINQEGITTYNNNVVATINLELNTYHQHNPKIFSRLPKLKLLYKQIGSPKED | |
| methionine) | KRIFTIEKRTEWQSLEDLIQKQNKVVEHEKKNVEILSNLKAAYISFFTNTDETILSEV | |
| | YFNKRSLNTISSFWFTGGWQTLLLKLKEFKLANQNKDGDIVVPKALSLAELKQVLD | |
| | SLEEQDPAVNHLFKEMYSDCYKENLWQTFIAIWQCEITSKFNLLEGYIQECNAVKE | |
| | DTFDKKKHKNIIKNICDTYLDIEQISKYIIVHESLPKYDALYDAVILYLQESSLRSYYD | |
| | AFRNLISKRPVNEEKVKLNFQNSTLLDGWDMNKESANLCVLLKNNIGEYFLAVMN | |
| | KKSNMVFDQKKNSALYSAGNESSFQKLEYKLLPGPNKMLPKVIFAKSNEKYFIIPE | |
| | EIVQIREEESFKKGKKFDKHALKTWIRFMQESIEKYPGWKTFDFTFKKPEEYEDVS | |
| | KFYKDVEEQGYKLNWKDINEEELLSLVEQKKVYLFQIKSKDIGETKEHGNKNLHTL | |
| | LFLELLKPENTSRLKLLGGGEMFYRAPSMEKVYKTVNEKQVLDSKGNPILEAKRY | |
| | YEPKFFLHFPIQVKGSENGYKTEMNPKILRAISTSKEVNIIGIDRGEKHLLYYYVIKP | |
| | DGTPITQGSLNTISLGLDKNQNPRLVDERTFKILERDSKGKPSKISDFESTGKKVD | |
| | YIDYHNILTYYETKRNIARRSWDTIGAIKNFKEGYLSQAIHQIYQLMLKYNAVVVLE | |
| | DLNTEFKAKRTAKVEKSVYEKFEIALAKKLNHLIIKGTDPAEAGSVINPYQLTPAITA | |
| | DTLSDFKKSKQWGPLFYIRANYTSTTDPITGWRKHIYIPSGASDKEIKTYFCKQGE | |
| | KEPLIQISYDTALTAFAFTYTHEGKEWTLHATKDTQRMRYDSKKRKMEPVEIFDRL | |
| | RELFIDFSFEESLTDQLEATLSFDWKTLAFLWTMLNQIRNTDREAEGNDGDFIQSP | |
| | VAPFYDSRDPENKTNGLPVNGDANGAFNIARKGAILIKRIQEYAKKDPTFEKMREK | |
| | DGLNLYISDAEWDTEIS | |
| Wildtype | MTNTSIFKTFTNQYSLSKTLRFELRPHPMTSGLDDIISLDTGIKKLYENEMKPLFDE | 62 |
| amino acid | LHFEFISQSLVQVSFPSEKLEVLLNKYRSLKDQKAKNIEKELEGPLQELRTIITDTFE | |
| sequence (with | STGNNWKKEWLQQGFKIKSSGYKVLTEEGILEVLSVRKKDKADAINKFKGFFTYF | |
| N-terminal | SGFNMNRENYYSSEDKKTAVAYRVINENLIRYMDNILLLQNVLAKAPEFKKFEDIL | |
| methionine) | SLTTFGKYINQEGITTYNNNVVATINLELNTYHQHNPKIFSRLPKLKLLYKQIGSPKE | |
| | DKRIFTIEKRTEWQSLEDLIQKQNKVVEHEKKNVEILSNLKAAYISFFTNTDETILSE | |
| | VYFNKRSLNTISSFWFTGGWQTLLLKLKEFKLANQNKDGDIVVPKALSLAELKQVL | |
| | DSLEEQDPAVNHLFKEMYSDCYKENLWQTFIAIWQCEITSKFNLLEGYIQECNAV | |
| | KEDTFDKKKHKNIIKNICDTYLDIEQISKYIIVHESLPKYDALYDAVILYLQESSLRSY | |
| | YDAFRNLISKRPVNEEKVKLNFQNSTLLDGWDMNKESANLCVLLKNNIGEYFLAV | |
| | MNKKSNMVFDQKKNSALYSAGNESSFQKLEYKLLPGPNKMLPKVIFAKSNEKYFII | |
| | PEEIVQIREEESFKKGKKFDKHALKTWIRFMQESIEKYPGWKTFDFTFKKPEEYED | |
| | VSKFYKDVEEQGYKLNWKDINEEELLSLVEQKKVYLFQIKSKDIGETKEHGNKNLH | |
| | TLLFLELLKPENTSRLKLLGGGEMFYRAPSMEKVYKTVNEKQVLDSKGNPILEAK | |
| | RYYEPKFFLHFPIQVKGSENGYKTEMNPKILRAISTSKEVNIIGIDRGEKHLLYYYVI | |
| | KPDGTPITQGSLNTISLGLDKNQNPRLVDERTFKILERDSKGKPSKISDFESTGKK | |
| | VDYIDYHNILTYYETKRNIARRSWDTIGAIKNFKEGYLSQAIHQIYQLMLKYNAVVV | |
| | LEDLNTEFKAKRTAKVEKSVYEKFEIALAKKLNHLIIKGTDPAEAGSVINPYQLTPAI | |
| | TADTLSDFKKSKQWGPLFYIRANYTSTTDPITGWRKHIYIPSGASDKEIKTYFCKQ | |
| | GEKEPLIQISYDTALTAFAFTYTHEGKEWTLHATKDTQRMRYDSKKRKMEPVEIF | |
| | DRLRELFIDFSFEESLTDQLEATLSFDWKTLAFLWTMLNQIRNTDREAEGNDGDFI | |
| | QSPVAPFYDSRDPENKTNGLPVNGDANGAFNIARKGAILIKRIQEYAKKDPTFEKM | |
| | REKDGLNLYISDAEWDTEIS | |
| Expression | MGSGTNTSIFKTFTNQYSLSKTLRFELRPHPMTSGLDDIISLDTGIKKLYENEMKPL | 63 |
| construct (with | FDELHFEFISQSLVQVSFPSEKLEVLLNKYRSLKDQKAKNIEKELEGPLQELRTIITD | |
| N-terminal | TFESTGNNWKKEWLQQGFKIKSSGYKVLTEEGILEVLSVRKKDKADAINKFKGFF | |
| methionine, | TYFSGFNMNRENYYSSEDKKTAVAYRVINENLIRYMDNILLLQNVLAKAPEFKKFE | |
| V5-tag and C- | DILSLTTFGKYINQEGITTYNNNVVATINLELNTYHQHNPKIFSRLPKLKLLYKQIGS | |
| terminal NLS) | PKEDKRIFTIEKRTEWQSLEDLIQKQNKVVEHEKKNVEILSNLKAAYISFFTNTDETI | |
| aa sequence | LSEVYFNKRSLNTISSFWFTGGWQTLLLKLKEFKLANQNKDGDIVVPKALSLAELK | |
| | QVLDSLEEQDPAVNHLFKEMYSDCYKENLWQTFIAIWQCEITSKFNLLEGYIQEC | |
| | NAVKEDTFDKKKHKNIIKNICDTYLDIEQISKYIIVHESLPKYDALYDAVILYLQESSL | |
| | RSYYDAFRNLISKRPVNEEKVKLNFQNSTLLDGWDMNKESANLCVLLKNNIGEYF | |
| | LAVMNKKSNMVFDQKKNSALYSAGNESSFQKLEYKLLPGPNKMLPKVIFAKSNEK | |
| | YFIIPEEIVQIREEESFKKGKKFDKHALKTWIRFMQESIEKYPGWKTFDFTFKKPEE | |
| | YEDVSKFYKDVEEQGYKLNWKDINEEELLSLVEQKKVYLFQIKSKDIGETKEHGN | |
| | KNLHTLLFLELLKPENTSRLKLLGGGEMFYRAPSMEKVYKTVNEKQVLDSKGNPIL | |
| | EAKRYYEPKFFLHFPIQVKGSENGYKTEMNPKILRAISTSKEVNIIGIDRGEKHLLY | |
| | YYVIKPDGTPITQGSLNTISLGLDKNQNPRLVDERTFKILERDSKGKPSKISDFEST | |
| | GKKVDYIDYHNILTYYETKRNIARRSWDTIGAIKNFKEGYLSQAIHQIYQLMLKYNA | |
| | WVVLEDLNTEFKAKRTAKVEKSVYEKFEIALAKKLNHLIIKGTDPAEAGSVINPYQL | |
| | TPAITADTLSDFKKSKQWGPLFYIRANYTSTTDPITGWRKHIYIPSGASDKEIKTYF | |
| | CKQGEKEPLIQISYDTALTAFAFTYTHEGKEWTLHATKDTQRMRYDSKKRKMEPV | |
| | EIFDRLRELFIDFSFEESLTDQLEATLSFDWKTLAFLWTMLNQIRNTDREAEGNDG | |
| | DFIQSPVAPFYDSRDPENKTNGLPVNGDANGAFNIARKGAILIKRIQEYAKKDPTF | |
| | EKMREKDGLNLYISDAEWDTEISSRKRTADGSEFESPKKKRKVGSGKPIPNPLLG | |
| | LDST | |
| Wildtype | ATGACTAACACATCTATTTTCAAAACCTTCACTAATCAATATTCACTTTCAAAAA | 64 |
| coding | CGTTGCGGTTTGAGTTGAGACCTCATCCGATGACTAGTGGTCTAGATGATATC | |
| sequence (with | ATTTCATTAGATACTGGCATAAAAAAATTGTATGAAAACGAGATGAAGCCGCTA | |
| N-terminal | TTTGATGAACTTCATTTTGAATTTATCTCTCAGTCGCTAGTTCAAGTATCATTCC | |
| methionine | CTTCAGAAAAACTGGAAGTTTTGCTAAACAAGTATAGGTCTCTTAAGGATCAG | |
| and stop | AAAGCTAAAAATATAGAAAAAGAACTGGAAGGCCCATTACAGGAACTAAGAAC | |
| codon) | AATTATTACTGACACCTTTGAATCCACTGGTAACAACTGGAAAAAAGAATGGCT | |
| | ACAACAAGGGTTTAAAATCAAAAGCTCGGGATACAAAGTACTAACAGAAGAGG | |
| | GAATATTAGAAGTATTGTCTGTTCGTAAAAAAGATAAAGCGGATGCAATCAATA | |
| | AATTTAAAGGATTCTTCACGTACTTTTCAGGGTTTAACATGAACCGTGAAAATT | |
| | ATTATTCATCGGAAGATAAAAAAACAGCTGTAGCGTATAGGGTAATTAATGAAA | |
| | ACCTTATCCGGTATATGGATAACATTCTCCTCCTTCAGAATGTTTTAGCAAAAG | |
| | CTCCTGAGTTTAAAAAGTTTGAAGATATTTTAAGTCTTACTACATTTGGAAAATA | |
| | CATAAATCAGGAAGGAATAACTACATATAATAATAACGTAGTTGCAACAATTAA | |
| | TCTTGAACTTAATACGTACCATCAGCATAATCCAAAAATCTTTTCTCGCCTGCC | |
| | AAAGTTAAAATTGCTTTATAAACAAATTGGTTCACCAAAAGAGGACAAACGCAT | |
| | TTTTACTATTGAAAAAAGAACGGAATGGCAGAGTTTGGAAGACTTAATACAAAA | |
| | ACAGAATAAAGTTGTTGAACACGAAAAAAAGAATGTTGAAATCCTGTCAAATTT | |
| | GAAAGCAGCATACATTTCTTTTTTCACGAACACAGATGAAACAATCTTAAGCGA | |
| | GGTATATTTCAATAAGCGTTCTCTTAATACAATTTCTTCTTTCTGGTTTACGGGT | |
| | GGCTGGCAAACACTGCTTCTTAAACTAAAAGAGTTTAAATTGGCCAATCAAAA | |
| | CAAAGATGGTGATATAGTAGTCCCTAAAGCATTATCCCTTGCTGAACTAAAAC | |
| | AGGTGCTTGATTCGTTAGAAGAGCAAGACCCTGCTGTTAATCATTTATTTAAG | |
| | GAAATGTACTCAGATTGTTACAAAGAAAACCTATGGCAGACCTTTATAGCTATC | |
| | TGGCAATGTGAAATTACATCAAAATTTAACCTGCTCGAAGGGTATATTCAAGAA | |
| | TGTAATGCTGTTAAAGAAGACACCTTTGATAAAAAAAAGCATAAAAATATTATC | |
| | AAAAACATCTGCGATACATACCTGGATATTGAGCAGATATCAAAATACATAATA | |
| | GTACATGAAAGTCTTCCTAAATATGATGCGCTATATGATGCGGTAATACTTTAT | |
| | TTGCAGGAATCTTCTTTACGCAGTTATTACGATGCCTTCCGCAACCTTATTAGC | |
| | AAGCGACCTGTTAACGAAGAAAAAGTTAAGCTCAACTTTCAGAACTCTACCCT | |
| | GCTTGATGGCTGGGATATGAATAAAGAAAGCGCTAACTTATGCGTATTACTGA | |
| | AAAACAATATAGGTGAATACTTCCTTGCTGTAATGAATAAAAAGAGCAACATGG | |
| | TTTTTGATCAGAAGAAAAACTCTGCCCTTTACTCTGCTGGGAATGAAAGTAGTT | |
| | TTCAGAAGCTGGAGTATAAACTGTTGCCTGGGCCTAACAAAATGCTGCCAAAA | |
| | GTAATTTTTGCAAAATCGAACGAAAAATATTTCATCATACCGGAAGAAATTGTG | |
| | CAGATTAGAGAAGAAGAATCGTTTAAAAAAGGAAAAAAATTTGATAAGCATGC | |
| | ATTGAAAACGTGGATCAGGTTTATGCAGGAATCAATTGAAAAATACCCAGGTT | |
| | GGAAGACATTCGACTTTACCTTTAAAAAACCGGAAGAGTACGAAGATGTCAGC | |
| | AAGTTCTATAAAGATGTAGAAGAACAGGGGTATAAACTAAACTGGAAAGATAT | |
| | TAACGAGGAAGAGCTCCTGTCACTTGTAGAACAAAAAAAAGTATATCTGTTTC | |
| | AGATAAAAAGCAAAGATATCGGAGAAACAAAGGAGCACGGCAACAAGAACCT | |
| | TCACACATTGTTATTTTTAGAACTCCTCAAACCGGAAAATACCAGCAGGTTAAA | |
| | GCTACTGGGCGGTGGCGAAATGTTTTATCGTGCGCCAAGTATGGAAAAGGTA | |
| | TACAAAACCGTAAATGAAAAACAGGTTCTGGATTCAAAAGGTAACCCCATTTTA | |
| | GAAGCAAAACGGTACTATGAACCAAAGTTTTTCCTTCACTTCCCTATTCAGGTC | |
| | AAAGGGAGCGAAAATGGTTATAAAACAGAAATGAATCCGAAAATATTGCGGGC | |
| | AATTAGCACTTCAAAAGAAGTAAATATAATAGGAATAGACCGTGGAGAAAAGC | |
| | ATTTACTCTATTATTACGTTATAAAGCCAGACGGAACTCCAATTACTCAAGGAA | |
| | GCCTGAATACAATTAGTTTAGGTTTAGATAAAAATCAAAATCCCAGACTTGTTG | |
| | ACGAGCGTACCTTCAAGATTTTGGAGAGAGATTCCAAGGGAAAACCATCAAAA | |
| | ATATCAGATTTTGAATCTACAGGGAAAAAAGTTGATTACATAGATTATCACAAT | |
| | ATACTTACCTATTACGAAACAAAACGCAATATAGCACGCCGTTCGTGGGATAC | |
| | TATTGGGGCAATAAAAAACTTTAAAGAGGGGTACTTGTCTCAGGCGATTCACC | |
| | AGATTTATCAGCTTATGTTGAAGTATAACGCTGTGGTAGTTTTGGAAGATCTTA | |
| | ATACGGAGTTTAAGGCAAAACGAACCGCAAAAGTTGAAAAATCCGTGTACGAA | |
| | AAGTTTGAAATTGCCCTTGCTAAAAAACTGAACCACTTAATTATTAAAGGAACT | |
| | GACCCTGCAGAAGCAGGAAGCGTAATAAATCCGTATCAGCTTACTCCAGCAAT | |
| | TACAGCTGATACATTAAGCGACTTTAAGAAATCAAAACAATGGGGTCCGCTTT | |
| | TCTATATTAGAGCAAACTATACCTCTACGACTGACCCTATAACCGGCTGGCGT | |
| | AAACACATATATATCCCGTCCGGAGCTTCAGATAAAGAAATTAAAACATATTTC | |
| | TGTAAACAGGGCGAAAAAGAACCTTTGATTCAGATTTCATATGATACAGCGCT | |
| | TACCGCGTTTGCATTTACCTATACCCATGAAGGCAAAGAATGGACATTACACG | |
| | CAACGAAAGATACTCAGCGTATGCGTTATGACAGTAAGAAGCGGAAGATGGA | |
| | ACCCGTAGAAATATTTGATAGACTACGAGAGCTTTTTATAGATTTTAGTTTCGA | |
| | AGAATCGTTAACAGATCAACTAGAAGCAACACTTTCCTTTGACTGGAAAACAC | |
| | TGGCCTTTTTGTGGACAATGTTAAACCAGATACGTAATACCGACAGAGAAGCA | |
| | GAAGGGAATGACGGTGACTTTATTCAGTCTCCGGTTGCTCCGTTTTATGATAG | |
| | TCGAGATCCGGAAAATAAAACAAATGGACTTCCTGTTAACGGAGATGCTAATG | |
| | GGGCTTTCAATATAGCCAGAAAAGGTGCAATCCTGATAAAACGTATTCAAGAA | |
| | TATGCAAAAAAAGACCCCACCTTTGAAAAGATGAGAGAAAAAGATGGTCTCAA | |
| | TTTGTATATATCTGATGCAGAGTGGGATACAGAAATAAGCTAA | |
| Codon | ACAAACACTAGCATCTTCAAGACATTCACCAACCAATACAGCCTCTCCAAGAC | 65 |
| optimized | CCTGCGGTTTGAGCTCAGACCCCACCCTATGACCTCCGGCCTGGACGACATC | |
| coding | ATCAGCCTGGACACCGGAATCAAAAAGCTGTACGAGAACGAAATGAAGCCTC | |
| sequence (no | TGTTCGACGAGCTGCACTTCGAGTTCATCAGCCAGAGCCTGGTCCAGGTCAG | |
| N-terminal | CTTCCCTAGCGAGAAGCTCGAAGTGCTGCTGAACAAGTACCGGAGCCTGAAG | |
| methionine, no | GACCAGAAAGCTAAGAACATCGAGAAGGAACTGGAGGGCCCCCTGCAGGAG | |
| stop codon) | CTGAGAACCATCATCACCGACACCTTCGAGAGCACCGGCAACAACTGGAAGA | |
| | AAGAGTGGCTGCAGCAGGGGTTCAAGATCAAAAGCAGTGGATACAAGGTGCT | |
| | GACAGAGGAGGGCATCCTGGAAGTGCTTTCCGTGCGGAAGAAGGATAAGGC | |
| | CGATGCTATAAACAAGTTCAAAGGATTCTTCACCTACTTCAGCGGCTTCAACA | |
| | TGAACAGAGAGAACTACTACAGCAGCGAAGATAAAAAAACAGCCGTGGCCTA | |
| | CAGAGTGATCAACGAGAACCTGATCCGGTACATGGATAACATCCTGCTCCTG | |
| | CAGAACGTGCTGGCCAAAGCCCCTGAGTTCAAGAAATTTGAAGATATCCTGA | |
| | GTCTGACCACCTTCGGCAAGTACATCAACCAGGAGGGCATCACAACCTACAA | |
| | CAACAACGTTGTGGCCACCATCAACCTGGAGCTGAACACCTACCACCAGCAC | |
| | AACCCAAAAATCTTCAGCAGACTGCCCAAACTGAAGCTGCTGTACAAGCAGAT | |
| | CGGTTCTCCAAAGGAGGACAAGCGCATCTTCACCATCGAGAAGAGAACAGAA | |
| | TGGCAGAGCCTGGAGGACCTGATCCAGAAGCAGAACAAGGTCGTGGAACAC | |
| | GAAAAGAAGAACGTGGAGATCCTGTCTAATCTGAAGGCCGCCTATATCAGCTT | |
| | CTTCACAAACACCGACGAAACCATCCTGTCTGAGGTGTACTTCAACAAGAGAA | |
| | GCCTGAATACGATCAGCAGCTTCTGGTTCACCGGCGGATGGCAAACCCTGCT | |
| | GCTGAAACTGAAGGAATTTAAGCTGGCTAATCAGAACAAAGACGGCGATATC | |
| | GTGGTTCCCAAGGCCCTGAGCCTGGCCGAGCTGAAGCAGGTGCTGGACTCC | |
| | CTGGAAGAGCAGGACCCCGCCGTGAATCACCTGTTCAAGGAAATGTACAGCG | |
| | ACTGCTACAAGGAAAACCTGTGGCAAACATTTATCGCCATCTGGCAATGTGAA | |
| | ATCACAAGCAAGTTCAACCTGCTGGAGGGCTATATCCAAGAGTGCAACGCCG | |
| | TGAAAGAGGACACCTTTGACAAGAAAAAGCACAAAAACATCATCAAGAACATC | |
| | TGCGACACGTACCTGGACATTGAGCAGATCAGTAAGTACATCATCGTGCACG | |
| | AAAGCCTGCCTAAATACGACGCCCTCTATGATGCCGTCATCCTGTACCTGCAG | |
| | GAGTCTAGTCTGCGGTCCTACTACGACGCCTTTAGAAACCTGATTTCTAAGCG | |
| | GCCAGTGAACGAGGAAAAGGTGAAGCTGAATTTCCAGAATAGCACCCTGCTG | |
| | GATGGCTGGGACATGAATAAAGAAAGCGCCAATCTTTGTGTGCTGCTGAAGA | |
| | ACAACATCGGAGAGTACTTTCTGGCCGTGATGAACAAAAAAAGCAACATGGTT | |
| | TTTGACCAGAAAAAAAACAGCGCCCTGTATAGCGCTGGCAATGAATCTAGCTT | |
| | CCAGAAGCTGGAGTACAAGCTGTTGCCCGGCCCTAACAAGATGCTGCCTAAG | |
| | GTGATCTTTGCCAAGTCCAATGAGAAGTACTTCATCATCCCTGAGGAGATCGT | |
| | GCAGATCAGGGAGGAAGAGAGCTTCAAGAAAGGCAAAAAATTCGATAAGCAC | |
| | GCGCTGAAAACCTGGATCAGATTCATGCAGGAGTCTATCGAGAAGTATCCTG | |
| | GCTGGAAAACCTTTGACTTCACATTCAAAAAGCCTGAGGAATACGAGGATGTG | |
| | TCCAAGTTCTACAAAGACGTGGAAGAGCAGGGCTACAAACTGAACTGGAAGG | |
| | ATATCAACGAGGAAGAACTGCTGAGCCTGGTGGAACAGAAGAAGGTGTACCT | |
| | TTTTCAGATCAAGTCCAAAGACATAGGCGAGACAAAGGAACACGGAAATAAGA | |
| | ACCTGCACACCCTGCTCTTCCTAGAATTGCTGAAGCCTGAGAACACAAGTCG | |
| | GCTGAAGCTGTTGGGCGGCGGAGAAATGTTCTACCGGGCCCCTTCTATGGAA | |
| | AAAGTCTACAAAACAGTGAACGAGAAGCAGGTGCTGGATTCTAAAGGCAACC | |
| | CTATCCTGGAGGCCAAGCGCTACTACGAGCCTAAGTTTTTTCTGCATTTCCCC | |
| | ATCCAGGTGAAGGGCTCTGAGAACGGCTATAAGACCGAGATGAACCCCAAAA | |
| | TCCTCAGAGCCATCAGCACCAGCAAGGAAGTGAACATCATTGGCATCGACAG | |
| | AGGCGAGAAGCACCTGCTGTACTATTACGTGATCAAGCCCGACGGAACACCT | |
| | ATCACCCAGGGCAGCCTGAACACCATCTCCCTGGGCCTTGATAAGAATCAAA | |
| | ATCCTAGACTGGTGGACGAGAGAACCTTCAAGATCCTGGAAAGAGATAGCAA | |
| | GGGCAAGCCAAGCAAGATCTCAGATTTTGAAAGCACAGGCAAGAAGGTCGAC | |
| | TACATCGACTACCACAACATCCTGACATACTATGAAACCAAGAGAAATATCGC | |
| | CAGAAGAAGCTGGGACACAATTGGCGCCATCAAGAATTTCAAGGAGGGATAC | |
| | CTCTCTCAGGCCATCCACCAGATCTACCAGCTGATGCTGAAATATAACGCCGT | |
| | GGTGGTGCTAGAGGACCTGAACACCGAGTTCAAGGCAAAGAGAACCGCCAA | |
| | GGTGGAAAAAAGCGTGTACGAAAAGTTTGAGATAGCTCTGGCCAAGAAGCTG | |
| | AATCACCTGATCATCAAGGGCACCGACCCAGCCGAGGCCGGATCTGTGATCA | |
| | ACCCTTACCAGCTGACCCCTGCTATTACAGCCGACACACTGAGCGATTTCAAG | |
| | AAGAGCAAACAATGGGGCCCTCTGTTCTACATCCGGGCCAACTACACCAGCA | |
| | CAACCGACCCTATCACAGGCTGGAGAAAGCACATCTACATCCCCAGCGGAGC | |
| | CAGTGACAAGGAAATCAAGACCTACTTCTGCAAGCAGGGCGAGAAGGAGCCT | |
| | CTGATCCAGATTAGCTACGACACCGCCCTGACCGCCTTCGCCTTCACATACA | |
| | CCCACGAAGGCAAGGAGTGGACCCTACATGCCACAAAGGATACCCAAAGAAT | |
| | GCGGTACGACAGCAAGAAGAGAAAGATGGAACCCGTGGAAATCTTCGACAGA | |
| | CTGAGAGAGCTGTTCATCGACTTCTCTTTCGAGGAAAGCCTGACCGACCAGC | |
| | TGGAGGCAACCCTGTCCTTCGACTGGAAAACCCTGGCTTTTCTGTGGACAAT | |
| | GCTGAATCAGATCAGAAACACCGATAGAGAGGCTGAAGGCAACGACGGCGA | |
| | CTTCATCCAGTCTCCTGTGGCCCCTTTCTATGATAGCCGGGACCCAGAGAAC | |
| | AAGACCAATGGCCTGCCCGTTAACGGCGACGCCAACGGCGCCTTCAACATCG | |
| | CTAGAAAGGGGGCTATCCTGATCAAGAGAATCCAGGAATACGCCAAGAAGGA | |
| | CCCTACATTCGAGAAGATGCGGGAAAAGGACGGTTTAAACCTGTACATCAGC | |
| | GATGCTGAGTGGGATACCGAGATCAGC | |
| Expression | ATGggctccggaACAAACACTAGCATCTTCAAGACATTCACCAACCAATACAGCCT | 66 |
| construct (with | CTCCAAGACCCTGCGGTTTGAGCTCAGACCCCACCCTATGACCTCCGGCCTG | |
| N-terminal | GACGACATCATCAGCCTGGACACCGGAATCAAAAAGCTGTACGAGAACGAAA | |
| methionine | TGAAGCCTCTGTTCGACGAGCTGCACTTCGAGTTCATCAGCCAGAGCCTGGT | |
| and stop | CCAGGTCAGCTTCCCTAGCGAGAAGCTCGAAGTGCTGCTGAACAAGTACCGG | |
| codon, | AGCCTGAAGGACCAGAAAGCTAAGAACATCGAGAAGGAACTGGAGGGCCCC | |
| includes V5- | CTGCAGGAGCTGAGAACCATCATCACCGACACCTTCGAGAGCACCGGCAACA | |
| tag and C- | ACTGGAAGAAAGAGTGGCTGCAGCAGGGGTTCAAGATCAAAAGCAGTGGATA | |
| terminal NLS) | CAAGGTGCTGACAGAGGAGGGCATCCTGGAAGTGCTTTCCGTGCGGAAGAA | |
| | GGATAAGGCCGATGCTATAAACAAGTTCAAAGGATTCTTCACCTACTTCAGCG | |
| | GCTTCAACATGAACAGAGAGAACTACTACAGCAGCGAAGATAAAAAAACAGCC | |
| | GTGGCCTACAGAGTGATCAACGAGAACCTGATCCGGTACATGGATAACATCC | |
| | TGCTCCTGCAGAACGTGCTGGCCAAAGCCCCTGAGTTCAAGAAATTTGAAGA | |
| | TATCCTGAGTCTGACCACCTTCGGCAAGTACATCAACCAGGAGGGCATCACA | |
| | ACCTACAACAACAACGTTGTGGCCACCATCAACCTGGAGCTGAACACCTACC | |
| | ACCAGCACAACCCAAAAATCTTCAGCAGACTGCCCAAACTGAAGCTGCTGTAC | |
| | AAGCAGATCGGTTCTCCAAAGGAGGACAAGCGCATCTTCACCATCGAGAAGA | |
| | GAACAGAATGGCAGAGCCTGGAGGACCTGATCCAGAAGCAGAACAAGGTCG | |
| | TGGAACACGAAAAGAAGAACGTGGAGATCCTGTCTAATCTGAAGGCCGCCTA | |
| | TATCAGCTTCTTCACAAACACCGACGAAACCATCCTGTCTGAGGTGTACTTCA | |
| | ACAAGAGAAGCCTGAATACGATCAGCAGCTTCTGGTTCACCGGCGGATGGCA | |
| | AACCCTGCTGCTGAAACTGAAGGAATTTAAGCTGGCTAATCAGAACAAAGACG | |
| | GCGATATCGTGGTTCCCAAGGCCCTGAGCCTGGCCGAGCTGAAGCAGGTGC | |
| | TGGACTCCCTGGAAGAGCAGGACCCCGCCGTGAATCACCTGTTCAAGGAAAT | |
| | GTACAGCGACTGCTACAAGGAAAACCTGTGGCAAACATTTATCGCCATCTGG | |
| | CAATGTGAAATCACAAGCAAGTTCAACCTGCTGGAGGGCTATATCCAAGAGTG | |
| | CAACGCCGTGAAAGAGGACACCTTTGACAAGAAAAAGCACAAAAACATCATCA | |
| | AGAACATCTGCGACACGTACCTGGACATTGAGCAGATCAGTAAGTACATCATC | |
| | GTGCACGAAAGCCTGCCTAAATACGACGCCCTCTATGATGCCGTCATCCTGT | |
| | ACCTGCAGGAGTCTAGTCTGCGGTCCTACTACGACGCCTTTAGAAACCTGATT | |
| | TCTAAGCGGCCAGTGAACGAGGAAAAGGTGAAGCTGAATTTCCAGAATAGCA | |
| | CCCTGCTGGATGGCTGGGACATGAATAAAGAAAGCGCCAATCTTTGTGTGCT | |
| | GCTGAAGAACAACATCGGAGAGTACTTTCTGGCCGTGATGAACAAAAAAAGC | |
| | AACATGGTTTTTGACCAGAAAAAAAACAGCGCCCTGTATAGCGCTGGCAATGA | |
| | ATCTAGCTTCCAGAAGCTGGAGTACAAGCTGTTGCCCGGCCCTAACAAGATG | |
| | CTGCCTAAGGTGATCTTTGCCAAGTCCAATGAGAAGTACTTCATCATCCCTGA | |
| | GGAGATCGTGCAGATCAGGGAGGAAGAGAGCTTCAAGAAAGGCAAAAAATTC | |
| | GATAAGCACGCGCTGAAAACCTGGATCAGATTCATGCAGGAGTCTATCGAGA | |
| | AGTATCCTGGCTGGAAAACCTTTGACTTCACATTCAAAAAGCCTGAGGAATAC | |
| | GAGGATGTGTCCAAGTTCTACAAAGACGTGGAAGAGCAGGGCTACAAACTGA | |
| | ACTGGAAGGATATCAACGAGGAAGAACTGCTGAGCCTGGTGGAACAGAAGAA | |
| | GGTGTACCTTTTTCAGATCAAGTCCAAAGACATAGGCGAGACAAAGGAACAC | |
| | GGAAATAAGAACCTGCACACCCTGCTCTTCCTAGAATTGCTGAAGCCTGAGAA | |
| | CACAAGTCGGCTGAAGCTGTTGGGCGGCGGAGAAATGTTCTACCGGGCCCC | |
| | TTCTATGGAAAAAGTCTACAAAACAGTGAACGAGAAGCAGGTGCTGGATTCTA | |
| | AAGGCAACCCTATCCTGGAGGCCAAGCGCTACTACGAGCCTAAGTTTTTTCTG | |
| | CATTTCCCCATCCAGGTGAAGGGCTCTGAGAACGGCTATAAGACCGAGATGA | |
| | ACCCCAAAATCCTCAGAGCCATCAGCACCAGCAAGGAAGTGAACATCATTGG | |
| | CATCGACAGAGGCGAGAAGCACCTGCTGTACTATTACGTGATCAAGCCCGAC | |
| | GGAACACCTATCACCCAGGGCAGCCTGAACACCATCTCCCTGGGCCTTGATA | |
| | AGAATCAAAATCCTAGACTGGTGGACGAGAGAACCTTCAAGATCCTGGAAAG | |
| | AGATAGCAAGGGCAAGCCAAGCAAGATCTCAGATTTTGAAAGCACAGGCAAG | |
| | AAGGTCGACTACATCGACTACCACAACATCCTGACATACTATGAAACCAAGAG | |
| | AAATATCGCCAGAAGAAGCTGGGACACAATTGGCGCCATCAAGAATTTCAAG | |
| | GAGGGATACCTCTCTCAGGCCATCCACCAGATCTACCAGCTGATGCTGAAAT | |
| | ATAACGCCGTGGTGGTGCTAGAGGACCTGAACACCGAGTTCAAGGCAAAGAG | |
| | AACCGCCAAGGTGGAAAAAAGCGTGTACGAAAAGTTTGAGATAGCTCTGGCC | |
| | AAGAAGCTGAATCACCTGATCATCAAGGGCACCGACCCAGCCGAGGCCGGAT | |
| | CTGTGATCAACCCTTACCAGCTGACCCCTGCTATTACAGCCGACACACTGAG | |
| | CGATTTCAAGAAGAGCAAACAATGGGGCCCTCTGTTCTACATCCGGGCCAAC | |
| | TACACCAGCACAACCGACCCTATCACAGGCTGGAGAAAGCACATCTACATCC | |
| | CCAGCGGAGCCAGTGACAAGGAAATCAAGACCTACTTCTGCAAGCAGGGCGA | |
| | GAAGGAGCCTCTGATCCAGATTAGCTACGACACCGCCCTGACCGCCTTCGCC | |
| | TTCACATACACCCACGAAGGCAAGGAGTGGACCCTACATGCCACAAAGGATA | |
| | CCCAAAGAATGCGGTACGACAGCAAGAAGAGAAAGATGGAACCCGTGGAAAT | |
| | CTTCGACAGACTGAGAGAGCTGTTCATCGACTTCTCTTTCGAGGAAAGCCTGA | |
| | CCGACCAGCTGGAGGCAACCCTGTCCTTCGACTGGAAAACCCTGGCTTTTCT | |
| | GTGGACAATGCTGAATCAGATCAGAAACACCGATAGAGAGGCTGAAGGCAAC | |
| | GACGGCGACTTCATCCAGTCTCCTGTGGCCCCTTTCTATGATAGCCGGGACC | |
| | CAGAGAACAAGACCAATGGCCTGCCCGTTAACGGCGACGCCAACGGCGCCT | |
| | TCAACATCGCTAGAAAGGGGGCTATCCTGATCAAGAGAATCCAGGAATACGC | |
| | CAAGAAGGACCCTACATTCGAGAAGATGCGGGAAAAGGACGGTTTAAACCTG | |
| | T | |
| | ACATCAGCGATGCTGAGTGGGATACCGAGATCAGCtctagaAAGCGGACAGCAG | |
| | ACGGCTCCGAATTTGAAAGCCCTAAGAAAAAGAGAAAGGTGggatccGGCAAAC | |
| | CTATCCCCAATCCCCTGCTGGGCCTGGACAGCACCTGA | |

In some embodiments a ZXHQ Type V Cas protein comprises an amino acid sequence of SEQ ID NO: 61, SEQ ID NO:62, or SEQ ID NO:63. In some embodiments, a ZXHQ Type V Cas protein has nickase activity, for example resulting from one or more amino acid substitutions relative to the sequence of SEQ ID NO:61, SEQ ID NO:62, or SEQ ID NO:63. In some embodiments, the one or more amino acid substitutions providing nickase activity comprise a D836 substitution, wherein the position of the D836 substitution is defined with respect to the amino acid numbering of SEQ ID NO:62 (corresponding to amino acid 908 of SEQ ID NO: 121). In some embodiments, the one or more amino acid substitutions providing nickase activity comprise an E963 substitution, wherein the position of the E963 substitution is defined with respect to the amino acid numbering of SEQ ID NO:62 (corresponding to amino acid 993 of SEQ ID NO: 121). In some embodiments, the one or more amino acid substitutions providing nickase activity comprise a R1172 substitution, wherein the position of the R1172 substitution is defined with respect to the amino acid numbering of SEQ ID NO:62 (corresponding to amino acid 1226 of SEQ ID NO: 121). In some embodiments, the one or more amino acid substitutions providing nickase activity comprise a D1211 substitution, wherein the position of the D1211 substitution is defined with respect to the amino acid numbering of SEQ ID NO:62 (corresponding to amino acid 1263 of SEQ ID NO:121). In some embodiments, a ZXHQ Type V Cas protein is catalytically inactive, for example due to a R1172 substitution in combination with a D836 substitution, a E963 substitution, and/or D1211 substitution.

### 6.2.12. ZQKH Type V Cas Proteins

In one aspect, the disclosure provides ZQKH Type V Cas proteins. ZQKH Type V Cas proteins can be further classified as Type V-A Cas proteins. The ZQKH Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:67. In some embodiments, the ZQKH Type V Cas proteins comprise an amino acid sequence that is at least 95%, at least 96%, at least 97%, at least 98%, or at least 99% identical to SEQ ID NO:67. In some embodiments, a ZQKH Type V Cas protein comprises an amino acid sequence that is identical to SEQ ID NO:67.

Exemplary ZQKH Type V Cas protein sequences and nucleotide sequences encoding exemplary ZQKH Type V Cas proteins are set forth in Table 1L.

TABLE 1L
ZQKH Type V Cas Sequences

| | | SEQ |
|---|---|---|
| | | ID |
| Name | Sequence | NO. |
| Wildtype | AYQVVKCLINDYCQNEIIAPQLQKVSCDNTWIVKLREFQEAANWEAQKIIQQDLIGIINK | 67 |
| amino acid | KLPKKFNSKALIEAIPDYLQGKSKEDLQRMLSGIHDYEIKVKNQNLQVAWNNGLEDFC | |
| sequence | NLCYQQFRGFSGYLDALSENLKFLFSGRKNGIAYRIVYQNLVTFERNRRAYESLILINE | |
| (without N- | TFRVQDEALLLNYSSSLTQEGINTYNERIGQLVKNLKEFGDTDRSFRNWHRRFKKLN | |
| terminal | KQILSPRVAPPWLARAYRSDEEMVMSLQSFLDEFNPLKPRLKQLIANLESYDEHIYYF | |
| methionine) | RKSLSLLSVTLRNDYKALDEELSIPQEQANCRSLSLSWIPFRQELINEIERIIDSSYTDIE | |
| | KCLASASEYLNTERAKRNDYRLDNTVSFTIKKLMDVFLSLYRAVKPLTGTGEEEDRNE | |
| | DFYDEFTTIWDVLQYVQKLYNAVFAWLNKKPYENNSYPAYLDEFTLLKNWKEKAAYI | |
| | KRNGKFYFIMFNGIDEQDIIEHRGDSAILYHVESQSPDRIKANLTKQFVFSKKANAGKG | |
| | RPNPSKAKFVRDNPEFQADWERVKTEAYKVAGNTEALAHAIRYFQRCLQSHPDYNR | |
| | FPFNFRPANDYTSLDDFVDSIKDKLFMMEETAINWSYVRQLAEEGTIYLFKLYNKDYA | |
| | KNRVGGSKPNLHTLYWEAMFSSENLRENNIKLEEPKLFYREVATNRDGELNMRLIPH | |
| | RYATDQLELHVPIHLNVNATASSDINMMVLDAIREGSIENVIGIDRGERNLLYYSVLRL | |
| | SDGEIVDQKSLNITFNDVDYHAKLSTKEEEIHDEQREWKAKTSIRKLKEGYLSQAIHQL | |
| | TSLIVKYHAVVVLEDLSEDFYSKRQKINKQIYQIFEKRLIEKLSYFVDKDAAEGQAGNIY | |
| | SALQLSSPNLVRKDNKKIFQNGIVFFVPPEYTSAIDPVTGFCNLFDKNRVRNICELLYR | |
| | FENICYNRKNDRFEFTWDYRNVMTYTRLEQDNISHLWTACSLGNRIEWSGSERNKN | |
| | RRCEIVNLTQSMKVLFEKHGIQYQTGKDVREAVCSIRNNDFKKELKRLFFLMLSLRNS | |
| | IVDGKVKKDYILSPVQNQRGSFFDSREYEELDNPKLPKCGDANGAYNIARKGILTIRKL | |
| | ENGNEKALTLDEWVISTQKGNIRM | |
| Wildtype | MAYQVVKCLINDYCQNEIIAPQLQKVSCDNTWIVKLREFQEAANWEAQKIIQQDLIGII | 68 |
| amino acid | NKKLPKKFNSKALIEAIPDYLQGKSKEDLQRMLSGIHDYEIKVKNQNLQVAWNNGLED | |
| sequence (with | FCNLCYQQFRGFSGYLDALSENLKFLFSGRKNGIAYRIVYQNLVTFERNRRAYESLILI | |
| N-terminal | NETFRVQDEALLLNYSSSLTQEGINTYNERIGQLVKNLKEFGDTDRSFRNWHRRFKK | |
| methionine) | LNKQILSPRVAPPWLARAYRSDEEMVMSLQSFLDEFNPLKPRLKQLIANLESYDEHIY | |
| | YFRKSLSLLSVTLRNDYKALDEELSIPQEQANCRSLSLSWIPFRQELINEIERIIDSSYT | |
| | DIEKCLASASEYLNTERAKRNDYRLDNTVSFTIKKLMDVFLSLYRAVKPLTGTGEEED | |
| | RNEDFYDEFTTIWDVLQYVQKLYNAVFAWLNKKPYENNSYPAYLDEFTLLKNWKEKA | |
| | AYIKRNGKFYFIMFNGIDEQDIIEHRGDSAILYHVESQSPDRIKANLTKQFVFSKKANA | |
| | GKGRPNPSKAKFVRDNPEFQADWERVKTEAYKVAGNTEALAHAIRYFQRCLQSHPD | |
| | YNRFPFNFRPANDYTSLDDFVDSIKDKLFMMEETAINWSYVRQLAEEGTIYLFKLYNK | |
| | DYAKNRVGGSKPNLHTLYWEAMFSSENLRENNIKLEEPKLFYREVATNRDGELNMR | |
| | LIPHRYATDQLELHVPIHLNVNATASSDINMMVLDAIREGSIENVIGIDRGERNLLYYSV | |
| | LRLSDGEIVDQKSLNITFNDVDYHAKLSTKEEEIHDEQREWKAKTSIRKLKEGYLSQAI | |
| | HQLTSLIVKYHAVVVLEDLSEDFYSKRQKINKQIYQIFEKRLIEKLSYFVDKDAAEGQA | |
| | GNIYSALQLSSPNLVRKDNKKIFQNGIVFFVPPEYTSAIDPVTGFCNLFDKNRVRNICE | |
| | LLYRFENICYNRKNDRFEFTWDYRNVMTYTRLEQDNISHLWTACSLGNRIEWSGSER | |
| | NKNRRCEIVNLTQSMKVLFEKHGIQYQTGKDVREAVCSIRNNDFKKELKRLFFLMLSL | |
| | RNSIVDGKVKKDYILSPVQNQRGSFFDSREYEELDNPKLPKCGDANGAYNIARKGILT | |
| | IRKLENGNEKALTLDEWVISTQKGNIRM | |
| Expression | MGSGAYQVVKCLINDYCQNEIIAPQLQKVSCDNTWIVKLREFQEAANWEAQKIIQQDL | 69 |
| construct (with | IGIINKKLPKKFNSKALIEAIPDYLQGKSKEDLQRMLSGIHDYEIKVKNQNLQVAWNNG | |
| N-terminal | LEDFCNLCYQQFRGFSGYLDALSENLKFLFSGRKNGIAYRIVYQNLVTFERNRRAYE | |
| methionine, | SLILINETFRVQDEALLLNYSSSLTQEGINTYNERIGQLVKNLKEFGDTDRSFRNWHR | |
| V5-tag and C- | RFKKLNKQILSPRVAPPWLARAYRSDEEMVMSLQSFLDEFNPLKPRLKQLIANLESYD | |
| terminal NLS) | EHIYYFRKSLSLLSVTLRNDYKALDEELSIPQEQANCRSLSLSWIPFRQELINEIERIIDS | |
| aa sequence | SYTDIEKCLASASEYLNTERAKRNDYRLDNTVSFTIKKLMDVFLSLYRAVKPLTGTGE | |
| | EEDRNEDFYDEFTTIWDVLQYVQKLYNAVFAWLNKKPYENNSYPAYLDEFTLLKNWK | |
| | EKAAYIKRNGKFYFIMFNGIDEQDIIEHRGDSAILYHVESQSPDRIKANLTKQFVFSKKA | |
| | NAGKGRPNPSKAKFVRDNPEFQADWERVKTEAYKVAGNTEALAHAIRYFQRCLQSH | |
| | PDYNRFPFNFRPANDYTSLDDFVDSIKDKLFMMEETAINWSYVRQLAEEGTIYLFKLY | |
| | NKDYAKNRVGGSKPNLHTLYWEAMFSSENLRENNIKLEEPKLFYREVATNRDGELN | |
| | MRLIPHRYATDQLELHVPIHLNVNATASSDINMMVLDAIREGSIENVIGIDRGERNLLYY | |
| | SVLRLSDGEIVDQKSLNITFNDVDYHAKLSTKEEEIHDEQREWKAKTSIRKLKEGYLS | |
| | QAIHQLTSLIVKYHAVVVLEDLSEDFYSKRQKINKQIYQIFEKRLIEKLSYFVDKDAAEG | |
| | QAGNIYSALQLSSPNLVRKDNKKIFQNGIVFFVPPEYTSAIDPVTGFCNLFDKNRVRNI | |
| | CELLYRFENICYNRKNDRFEFTWDYRNVMTYTRLEQDNISHLWTACSLGNRIEWSGS | |
| | ERNKNRRCEIVNLTQSMKVLFEKHGIQYQTGKDVREAVCSIRNNDFKKELKRLFFLML | |
| | SLRNSIVDGKVKKDYILSPVQNQRGSFFDSREYEELDNPKLPKCGDANGAYNIARKGI | |
| | LTIRKLENGNEKALTLDEWVISTQKGNIRMSRKRTADGSEFESPKKKRKVGSGKPIPN | |
| | PLLGLDST | |
| Wildtype | ATGGCATACCAAGTGGTTAAATGCCTAATCAACGACTATTGCCAGAATGAAATCAT | 70 |
| coding | TGCACCTCAATTGCAGAAAGTTTCCTGTGATAACACTTGGATTGTAAAACTTCGCG | |
| sequence (with | AGTTTCAAGAGGCTGCCAATTGGGAAGCCCAAAAAATTATCCAGCAAGATCTTAT | |
| N-terminal | TGGTATCATAAACAAGAAACTTCCTAAAAAGTTCAATAGCAAGGCATTGATAGAAG | |
| methionine | CCATTCCTGACTATTTACAAGGCAAGTCTAAAGAAGATCTGCAACGTATGTTGAGT | |
| and stop | GGTATACATGACTATGAGATTAAGGTAAAAAATCAGAACCTTCAGGTGGCTTGGA | |
| codon) | ATAATGGGTTAGAAGATTTTTGTAACCTCTGCTATCAACAATTTAGAGGATTTTCT | |
| | GGCTATCTTGACGCTTTATCTGAGAACCTGAAATTTCTATTCTCGGGCAGAAAAAA | |
| | TGGTATAGCCTATAGAATAGTGTATCAGAACCTTGTTACATTTGAGAGGAATAGGA | |
| | GAGCTTATGAATCCCTAATATTAATAAATGAGACTTTTAGGGTACAAGATGAGGCT | |
| | CTACTTCTTAATTACTCCAGTAGTCTGACCCAAGAAGGTATCAACACCTATAATGA | |
| | ACGAATAGGGCAACTTGTCAAAAATCTGAAAGAATTTGGCGATACAGACAGATCT | |
| | TTCAGAAACTGGCATCGCCGATTCAAGAAACTGAACAAGCAAATCCTAAGCCCTC | |
| | GTGTTGCTCCACCTTGGTTGGCACGCGCCTACAGAAGCGATGAAGAGATGGTGA | |
| | TGTCGCTACAGTCTTTTCTCGACGAGTTCAATCCATTAAAACCTCGTTTGAAGCAA | |
| | CTTATTGCTAATCTGGAATCTTACGATGAGCATATCTATTACTTCCGCAAGTCTCT | |
| | TTCTCTATTATCGGTGACCTTGAGGAATGATTATAAGGCACTTGATGAAGAACTCT | |
| | CAATACCACAAGAACAGGCCAATTGCAGAAGTTTAAGCCTTTCGTGGATTCCGTT | |
| | TCGCCAAGAATTGATAAACGAAATAGAACGAATTATTGACAGTTCATATACAGACA | |
| | TAGAGAAGTGTCTTGCCTCTGCCTCGGAATATCTGAACACGGAGAGAGCAAAAC | |
| | GGAACGACTATCGTCTAGATAATACTGTGTCTTTCACAATCAAGAAACTGATGGA | |
| | CGTATTCCTGTCATTGTATCGTGCGGTGAAGCCTCTGACTGGAACAGGAGAGGA | |
| | GGAGGATCGAAACGAGGACTTCTATGATGAGTTTACAACAATCTGGGATGTGCTT | |
| | CAATATGTACAAAAACTTTATAATGCAGTTTTTGCATGGCTGAACAAGAAGCCTTA | |
| | TGAGAACAACAGCTATCCTGCCTATTTGGACGAGTTTACACTTCTTAAAAACTGGA | |
| | AGGAGAAAGCCGCGTATATAAAACGGAATGGGAAGTTCTATTTTATCATGTTCAAT | |
| | GGTATTGATGAACAAGACATTATCGAGCATCGAGGTGATTCTGCAATCTTGTATC | |
| | ATGTGGAAAGTCAATCCCCCGATAGGATTAAGGCAAATCTCACCAAACAATTTGT | |
| | TTTTTCCAAAAAAGCAAATGCAGGAAAGGGGCGACCAAATCCTTCTAAAGCCAAA | |
| | TTCGTGCGTGACAATCCAGAATTCCAAGCTGACTGGGAACGTGTGAAAACTGAAG | |
| | CATATAAAGTAGCTGGAAACACAGAAGCGCTTGCTCATGCCATTCGATATTTTCAA | |
| | CGCTGCCTTCAATCACATCCTGACTATAATAGGTTTCCGTTCAATTTTAGACCAGC | |
| | GAATGACTACACTAGTTTAGATGATTTTGTTGACTCCATTAAAGACAAATTGTTTAT | |
| | GATGGAAGAAACTGCTATTAACTGGTCGTATGTGAGGCAATTAGCAGAAGAAGGA | |
| | ACAATTTACTTGTTTAAACTCTACAATAAAGATTATGCCAAGAATAGAGTTGGCGG | |
| | GTCTAAACCCAACTTGCATACGCTCTATTGGGAGGCGATGTTCAGCTCTGAGAAC | |
| | CTTCGTGAAAATAATATAAAGTTGGAGGAACCCAAACTCTTCTATCGTGAAGTTGC | |
| | AACTAACCGTGATGGTGAATTGAATATGCGCTTGATACCTCACAGATATGCAACA | |
| | GACCAACTTGAGCTGCATGTTCCAATTCACTTAAATGTGAATGCAACCGCTTCAA | |
| | GCGATATAAATATGATGGTGTTGGATGCAATACGAGAAGGGAGTATTGAAAATGT | |
| | CATTGGTATTGACCGTGGAGAGAGGAACCTTCTCTACTATTCAGTCTTGCGGTTG | |
| | TCAGATGGTGAAATTGTTGACCAAAAAAGTTTGAATATTACTTTCAATGATGTTGA | |
| | CTACCACGCCAAACTGTCGACTAAAGAGGAGGAAATCCATGACGAACAAAGAGA | |
| | ATGGAAAGCAAAAACAAGTATTCGGAAACTGAAAGAAGGATACCTTAGTCAAGCT | |
| | ATCCACCAACTAACATCGCTGATTGTCAAGTACCATGCTGTGGTAGTGCTAGAAG | |
| | ACTTATCAGAGGACTTCTATTCGAAGCGCCAGAAGATAAACAAGCAAATCTATCA | |
| | GATATTTGAAAAAAGGCTGATAGAAAAACTGAGTTATTTTGTCGATAAGGATGCTG | |
| | CAGAAGGTCAGGCAGGCAATATATATTCAGCATTGCAGTTGTCAAGCCCCAACTT | |
| | GGTGAGGAAAGATAATAAAAAAATCTTTCAGAACGGCATCGTCTTTTTTGTGCCAC | |
| | CTGAATATACAAGTGCCATTGACCCTGTAACAGGGTTCTGCAATCTCTTTGACAA | |
| | GAATCGGGTAAGAAATATTTGCGAACTTCTCTACAGATTTGAAAACATCTGCTATA | |
| | ATAGGAAAAATGACCGATTTGAGTTCACATGGGACTATCGTAATGTTATGACTTAT | |
| | ACGCGTCTGGAGCAGGACAATATTTCACATCTTTGGACAGCATGCTCTTTAGGAA | |
| | ACAGGATTGAATGGTCTGGTAGCGAACGTAATAAAAACAGAAGGTGCGAAATTGT | |
| | AAACCTTACGCAATCTATGAAAGTTTTGTTTGAAAAACATGGTATCCAATACCAAA | |
| | CAGGAAAAGATGTAAGGGAGGCTGTATGCAGCATAAGAAACAACGATTTTAAAAA | |
| | AGAATTGAAGCGCCTGTTCTTCTTGATGTTATCTTTAAGGAATAGCATTGTTGATG | |
| | GAAAAGTGAAAAAAGACTATATATTATCCCCCGTTCAGAACCAACGAGGCAGTTT | |
| | TTTCGATAGTAGAGAATATGAAGAGTTGGACAATCCAAAACTCCCTAAATGTGGA | |
| | GATGCAAATGGCGCATATAATATTGCAAGGAAAGGGATACTGACAATTAGAAAGT | |
| | TGGAAAATGGCAATGAAAAGGCATTAACCCTTGATGAGTGGGTTATTTCTACGCA | |
| | AAAAGGGAATATACGCATGTAA | |
| Codon | GCCTACCAGGTGGTGAAATGCCTGATTAACGACTACTGCCAGAACGAGATCATC | 71 |
| optimized | GCCCCTCAGCTGCAAAAGGTGAGCTGCGACAATACCTGGATCGTGAAGCTCAGA | |
| coding | GAGTTCCAGGAGGCCGCAAACTGGGAAGCCCAGAAGATCATCCAGCAGGACCT | |
| sequence (no | GATCGGCATTATCAATAAGAAACTGCCTAAGAAATTCAACTCTAAGGCCCTGATC | |
| N-terminal | GAGGCTATACCTGATTACCTCCAGGGCAAGAGCAAGGAAGATCTGCAGAGAATG | |
| methionine, no | CTGTCCGGCATCCACGACTATGAGATCAAGGTGAAGAACCAGAACCTGCAGGTA | |
| stop codon) | GCTTGGAACAATGGCCTGGAAGATTTCTGTAACTTGTGCTACCAACAATTTAGAG | |
| | GCTTTTCCGGCTACCTTGATGCTCTGTCAGAAAATCTGAAGTTCCTGTTCAGCGG | |
| | CAGAAAAAACGGCATCGCCTACAGGATCGTCTACCAGAACCTGGTGACCTTCGA | |
| | GCGGAACCGGAGAGCTTACGAGAGCCTGATCCTGATCAACGAGACATTTAGAGT | |
| | GCAGGACGAGGCCCTGCTGCTCAACTACTCTAGCTCTCTGACACAGGAGGGAAT | |
| | CAACACGTACAACGAGCGGATCGGCCAGCTGGTGAAGAACCTGAAGGAGTTCG | |
| | GCGACACCGACCGGAGCTTTCGGAACTGGCACAGACGGTTCAAGAAACTGAACA | |
| | AGCAGATCCTGAGCCCTAGAGTGGCCCCTCCTTGGCTGGCTCGTGCCTACAGAA | |
| | GCGATGAGGAAATGGTGATGAGCCTGCAGAGCTTCCTGGATGAGTTCAACCCTC | |
| | TGAAACCTAGACTCAAACAGCTGATCGCCAATCTGGAGTCCTACGACGAGCACAT | |
| | CTACTACTTCAGAAAGTCCCTGTCTCTGCTGTCAGTGACACTGAGGAACGACTAT | |
| | AAGGCACTGGATGAAGAGCTGAGCATCCCTCAGGAGCAGGCCAACTGCAGATCT | |
| | CTTAGCCTGAGCTGGATTCCTTTCAGACAGGAACTGATCAACGAGATCGAGAGAA | |
| | TCATCGATAGCAGCTACACAGACATTGAGAAGTGCCTGGCCAGCGCCTCCGAGT | |
| | ACCTGAACACCGAGAGAGCCAAGAGAAACGACTACCGGCTAGATAATACCGTGT | |
| | CCTTCACCATCAAGAAGCTGATGGACGTGTTCCTGAGCCTGTACCGCGCCGTGA | |
| | AGCCTCTGACCGGAACAGGCGAAGAGGAGGACAGAAATGAAGATTTCTACGACG | |
| | AGTTCACCACCATCTGGGATGTGCTGCAATACGTGCAGAAGCTGTACAACGCTGT | |
| | TTTCGCCTGGCTGAACAAGAAGCCCTACGAGAACAATAGCTACCCTGCCTACCTG | |
| | GATGAATTTACCCTGCTGAAGAACTGGAAGGAAAAGGCCGCCTACATCAAGAGG | |
| | AATGGAAAATTCTACTTCATCATGTTCAACGGCATCGACGAGCAGGATATCATCG | |
| | AACACAGAGGAGATTCTGCCATCCTGTACCATGTGGAAAGCCAGAGCCCTGATA | |
| | GAATCAAGGCCAATCTGACCAAGCAGTTCGTGTTCAGCAAGAAAGCCAATGCCG | |
| | GCAAGGGCCGGCCCAATCCCAGCAAGGCCAAGTTCGTGAGAGATAACCCCGAG | |
| | TTTCAGGCCGACTGGGAGCGGGTGAAAACCGAGGCCTACAAGGTGGCCGGAAA | |
| | CACCGAGGCCCTGGCCCACGCCATCAGATACTTCCAAAGATGCCTGCAAAGCCA | |
| | CCCCGATTATAATCGGTTCCCCTTCAACTTCAGACCTGCCAACGACTACACATCT | |
| | CTGGATGACTTCGTGGACAGCATCAAGGACAAGCTGTTCATGATGGAAGAAACC | |
| | GCCATCAACTGGAGTTATGTGAGACAGCTGGCCGAAGAAGGCACAATCTACCTG | |
| | TTCAAGCTGTATAACAAAGACTACGCCAAGAACCGGGTGGGCGGCAGCAAGCCT | |
| | AACCTGCACACCCTGTACTGGGAGGCCATGTTCAGCTCTGAGAATCTGAGAGAA | |
| | AACAACATCAAACTGGAAGAACCCAAACTGTTCTACAGAGAGGTGGCCACAAACC | |
| | GGGACGGCGAGCTGAACATGAGACTGATCCCCCACAGATACGCCACCGACCAG | |
| | CTGGAACTGCACGTGCCTATCCACCTGAATGTGAACGCCACAGCCAGCAGCGAC | |
| | ATCAACATGATGGTCCTTGATGCCATCCGGGAAGGATCTATTGAGAACGTGATCG | |
| | GCATCGACCGGGGAGAACGGAACCTGCTGTACTACAGCGTCCTGCGACTGTCC | |
| | GACGGCGAGATCGTGGACCAGAAGAGCCTGAATATCACCTTTAACGATGTGGAC | |
| | TACCACGCAAAGTTGTCTACCAAGGAGGAAGAAATCCATGATGAGCAGAGAGAG | |
| | TGGAAAGCCAAGACCTCCATCAGAAAGCTGAAGGAAGGTTACCTGTCTCAGGCT | |
| | ATCCACCAGCTGACCAGCCTGATCGTGAAGTACCACGCTGTGGTAGTGCTGGAA | |
| | GATCTGAGCGAAGATTTCTACAGCAAGCGGCAGAAAATCAACAAGCAGATCTACC | |
| | AGATTTTCGAGAAAAGACTTATCGAGAAGCTGAGCTACTTTGTGGACAAAGACGC | |
| | CGCCGAGGGCCAGGCAGGCAACATCTACAGCGCCCTGCAGCTGAGCTCCCCAA | |
| | ATCTGGTGAGAAAGGACAACAAGAAGATCTTCCAGAACGGCATCGTGTTCTTCGT | |
| | GCCACCTGAGTACACGAGTGCGATTGACCCCGTGACCGGCTTCTGCAACCTGTT | |
| | TGACAAGAACAGAGTGCGCAATATCTGTGAGCTGCTCTACAGATTCGAAAACATT | |
| | TGCTACAACAGAAAGAATGACCGGTTTGAGTTCACATGGGACTATAGAAACGTGA | |
| | TGACCTACACCAGACTTGAGCAGGACAACATCTCTCACCTGTGGACCGCTTGTAG | |
| | CCTCGGCAACCGGATCGAGTGGAGCGGCTCTGAAAGAAATAAGAACAGAAGATG | |
| | CGAGATCGTGAACCTGACACAAAGCATGAAGGTCCTGTTTGAGAAGCACGGCAT | |
| | CCAGTACCAGACCGGCAAGGACGTGCGGGAAGCTGTGTGTAGTATCAGAAACAA | |
| | CGACTTTAAGAAAGAACTGAAGAGACTGTTTTTCCTGATGCTGAGCCTGCGTAAC | |
| | AGCATCGTGGATGGAAAGGTGAAAAAGGACTACATCCTGAGCCCAGTGCAAAAC | |
| | CAGCGGGGTAGCTTTTTCGACTCCAGAGAATATGAAGAACTGGACAACCCGAAG | |
| | TTGCCTAAGTGCGGGGACGCCAACGGCGCCTACAACATCGCCAGAAAAGGAATC | |
| | CTGACAATCAGAAAGCTGGAGAACGGCAACGAGAAAGCCCTGACCCTGGACGAA | |
| | TGGGTGATCAGCACCCAGAAGGGCAACATCAGAATG | |
| Expression | ATGggctccggaGCCTACCAGGTGGTGAAATGCCTGATTAACGACTACTGCCAGAAC | 72 |
| construct (with | GAGATCATCGCCCCTCAGCTGCAAAAGGTGAGCTGCGACAATACCTGGATCGTG | |
| N-terminal | AAGCTCAGAGAGTTCCAGGAGGCCGCAAACTGGGAAGCCCAGAAGATCATCCAG | |
| methionine | CAGGACCTGATCGGCATTATCAATAAGAAACTGCCTAAGAAATTCAACTCTAAGG | |
| and stop | CCCTGATCGAGGCTATACCTGATTACCTCCAGGGCAAGAGCAAGGAAGATCTGC | |
| codon, | AGAGAATGCTGTCCGGCATCCACGACTATGAGATCAAGGTGAAGAACCAGAACC | |
| includes V5- | TGCAGGTAGCTTGGAACAATGGCCTGGAAGATTTCTGTAACTTGTGCTACCAACA | |
| tag and C- | ATTTAGAGGCTTTTCCGGCTACCTTGATGCTCTGTCAGAAAATCTGAAGTTCCTGT | |
| terminal NLS) | TCAGCGGCAGAAAAAACGGCATCGCCTACAGGATCGTCTACCAGAACCTGGTGA | |
| | CCTTCGAGCGGAACCGGAGAGCTTACGAGAGCCTGATCCTGATCAACGAGACAT | |
| | TTAGAGTGCAGGACGAGGCCCTGCTGCTCAACTACTCTAGCTCTCTGACACAGG | |
| | AGGGAATCAACACGTACAACGAGCGGATCGGCCAGCTGGTGAAGAACCTGAAG | |
| | GAGTTCGGCGACACCGACCGGAGCTTTCGGAACTGGCACAGACGGTTCAAGAAA | |
| | CTGAACAAGCAGATCCTGAGCCCTAGAGTGGCCCCTCCTTGGCTGGCTCGTGCC | |
| | TACAGAAGCGATGAGGAAATGGTGATGAGCCTGCAGAGCTTCCTGGATGAGTTC | |
| | AACCCTCTGAAACCTAGACTCAAACAGCTGATCGCCAATCTGGAGTCCTACGACG | |
| | AGCACATCTACTACTTCAGAAAGTCCCTGTCTCTGCTGTCAGTGACACTGAGGAA | |
| | CGACTATAAGGCACTGGATGAAGAGCTGAGCATCCCTCAGGAGCAGGCCAACTG | |
| | CAGATCTCTTAGCCTGAGCTGGATTCCTTTCAGACAGGAACTGATCAACGAGATC | |
| | GAGAGAATCATCGATAGCAGCTACACAGACATTGAGAAGTGCCTGGCCAGCGCC | |
| | TCCGAGTACCTGAACACCGAGAGAGCCAAGAGAAACGACTACCGGCTAGATAAT | |
| | ACCGTGTCCTTCACCATCAAGAAGCTGATGGACGTGTTCCTGAGCCTGTACCGC | |
| | GCCGTGAAGCCTCTGACCGGAACAGGCGAAGAGGAGGACAGAAATGAAGATTTC | |
| | TACGACGAGTTCACCACCATCTGGGATGTGCTGCAATACGTGCAGAAGCTGTAC | |
| | AACGCTGTTTTCGCCTGGCTGAACAAGAAGCCCTACGAGAACAATAGCTACCCTG | |
| | CCTACCTGGATGAATTTACCCTGCTGAAGAACTGGAAGGAAAAGGCCGCCTACAT | |
| | CAAGAGGAATGGAAAATTCTACTTCATCATGTTCAACGGCATCGACGAGCAGGAT | |
| | ATCATCGAACACAGAGGAGATTCTGCCATCCTGTACCATGTGGAAAGCCAGAGC | |
| | CCTGATAGAATCAAGGCCAATCTGACCAAGCAGTTCGTGTTCAGCAAGAAAGCCA | |
| | ATGCCGGCAAGGGCCGGCCCAATCCCAGCAAGGCCAAGTTCGTGAGAGATAAC | |
| | CCCGAGTTTCAGGCCGACTGGGAGCGGGTGAAAACCGAGGCCTACAAGGTGGC | |
| | CGGAAACACCGAGGCCCTGGCCCACGCCATCAGATACTTCCAAAGATGCCTGCA | |
| | AAGCCACCCCGATTATAATCGGTTCCCCTTCAACTTCAGACCTGCCAACGACTAC | |
| | ACATCTCTGGATGACTTCGTGGACAGCATCAAGGACAAGCTGTTCATGATGGAAG | |
| | AAACCGCCATCAACTGGAGTTATGTGAGACAGCTGGCCGAAGAAGGCACAATCT | |
| | ACCTGTTCAAGCTGTATAACAAAGACTACGCCAAGAACCGGGTGGGCGGCAGCA | |
| | AGCCTAACCTGCACACCCTGTACTGGGAGGCCATGTTCAGCTCTGAGAATCTGA | |
| | GAGAAAACAACATCAAACTGGAAGAACCCAAACTGTTCTACAGAGAGGTGGCCA | |
| | CAAACCGGGACGGCGAGCTGAACATGAGACTGATCCCCCACAGATACGCCACC | |
| | GACCAGCTGGAACTGCACGTGCCTATCCACCTGAATGTGAACGCCACAGCCAGC | |
| | AGCGACATCAACATGATGGTCCTTGATGCCATCCGGGAAGGATCTATTGAGAAC | |
| | GTGATCGGCATCGACCGGGGAGAACGGAACCTGCTGTACTACAGCGTCCTGCG | |
| | ACTGTCCGACGGCGAGATCGTGGACCAGAAGAGCCTGAATATCACCTTTAACGA | |
| | TGTGGACTACCACGCAAAGTTGTCTACCAAGGAGGAAGAAATCCATGATGAGCA | |
| | GAGAGAGTGGAAAGCCAAGACCTCCATCAGAAAGCTGAAGGAAGGTTACCTGTC | |
| | TCAGGCTATCCACCAGCTGACCAGCCTGATCGTGAAGTACCACGCTGTGGTAGT | |
| | GCTGGAAGATCTGAGCGAAGATTTCTACAGCAAGCGGCAGAAAATCAACAAGCA | |
| | GATCTACCAGATTTTCGAGAAAAGACTTATCGAGAAGCTGAGCTACTTTGTGGAC | |
| | AAAGACGCCGCCGAGGGCCAGGCAGGCAACATCTACAGCGCCCTGCAGCTGAG | |
| | CTCCCCAAATCTGGTGAGAAAGGACAACAAGAAGATCTTCCAGAACGGCATCGT | |
| | GTTCTTCGTGCCACCTGAGTACACGAGTGCGATTGACCCCGTGACCGGCTTCTG | |
| | CAACCTGTTTGACAAGAACAGAGTGCGCAATATCTGTGAGCTGCTCTACAGATTC | |
| | GAAAACATTTGCTACAACAGAAAGAATGACCGGTTTGAGTTCACATGGGACTATA | |
| | GAAACGTGATGACCTACACCAGACTTGAGCAGGACAACATCTCTCACCTGTGGA | |
| | CCGCTTGTAGCCTCGGCAACCGGATCGAGTGGAGCGGCTCTGAAAGAAATAAGA | |
| | ACAGAAGATGCGAGATCGTGAACCTGACACAAAGCATGAAGGTCCTGTTTGAGA | |
| | AGCACGGCATCCAGTACCAGACCGGCAAGGACGTGCGGGAAGCTGTGTGTAGT | |
| | ATCAGAAACAACGACTTTAAGAAAGAACTGAAGAGACTGTTTTTCCTGATGCTGA | |
| | GCCTGCGTAACAGCATCGTGGATGGAAAGGTGAAAAAGGACTACATCCTGAGCC | |
| | CAGTGCAAAACCAGCGGGGTAGCTTTTTCGACTCCAGAGAATATGAAGAACTGG | |
| | ACAACCCGAAGTTGCCTAAGTGCGGGGACGCCAACGGCGCCTACAACATCGCCA | |
| | GAAAAGGAATCCTGACAATCAGAAAGCTGGAGAACGGCAACGAGAAAGCCCTGA | |
| | CCCTGGACGAATGGGTGATCAGCACCCAGAAGGGCAACATCAGAATGtctagaAAG | |
| | CGGACAGCAGACGGCTCCGAATTTGAAAGCCCTAAGAAAAAGAGAAAGGTGggat | |
| | ccGGCAAACCTATCCCCAATCCCCTGCTGGGCCTGGACAGCACCTGA | |

In some embodiments a ZQKH Type V Cas protein comprises an amino acid sequence of SEQ ID NO: 67, SEQ ID NO:68, or SEQ ID NO:69. In some embodiments, a ZQKH Type V Cas protein has nickase activity, for example resulting from one or more amino acid substitutions relative to the sequence of SEQ ID NO:67, SEQ ID NO:68, or SEQ ID NO:69. In some embodiments, the one or more amino acid substitutions providing nickase activity comprise a D744 substitution, wherein the position of the D744 substitution is defined with respect to the amino acid numbering of SEQ ID NO:68 (corresponding to amino acid 908 of SEQ ID NO: 121). In some embodiments, the one or more amino acid substitutions providing nickase activity comprise an E831 substitution, wherein the position of the E831 substitution is defined with respect to the amino acid numbering of SEQ ID NO:68 (corresponding to amino acid 993 of SEQ ID NO: 121). In some embodiments, the one or more amino acid substitutions providing nickase activity comprise a R1048 substitution, wherein the position of the R1048 substitution is defined with respect to the amino acid numbering of SEQ ID NO:68 (corresponding to amino acid 1226 of SEQ ID NO: 121). In some embodiments, the one or more amino acid substitutions providing nickase activity comprise a D1091 substitution, wherein the position of the D1091 substitution is defined with respect to the amino acid numbering of SEQ ID NO:68 (corresponding to amino acid 1263 of SEQ ID NO:121). In some embodiments, a ZQKH Type V Cas protein is catalytically inactive, for example due to a R1048 substitution in combination with a D744 substitution, a E831 substitution, and/or D1091 substitution.

### 6.2.13. ZRGM Type V Cas Protein

In one aspect, the disclosure provides ZRGM Type V Cas proteins. ZRGM Type V Cas proteins can be further classified as Type V-A Cas proteins. The ZRGM Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:73. In some embodiments, the ZRGM Type V Cas proteins comprise an amino acid sequence that is at least 95%, at least 96%, at least 97%, at least 98%, or at least 99% identical to SEQ ID NO:73. In some embodiments, a ZRGM Type V Cas protein comprises an amino acid sequence that is identical to SEQ ID NO:73.

Exemplary ZRGM Type V Cas protein sequences and nucleotide sequences encoding exemplary ZRGM Type V Cas proteins are set forth in Table 1M.

TABLE 1M
ZRGM Type V Cas Sequences

| | | SEQ ID |
|---|---|---|
| Name | Sequence | NO. |
| Wildtype | ERMYEEFRNCYSVRKTLSFKAIPTEETKKHLQLQWEVLGDEIRFENYDKMKMVLDQ | 73 |
| amino acid | LHQSYISRKLDNIGEENQKKIVEILEKLVLVMKKIDTTHQKDKEKAQNQLQSLQASLR | |
| sequence | KEIGMFFPKNEWQQLQGKNVFKKDGVLSEYNISEENKKNIQCYDGFMTFFKKYNET | |
| (without N- | RANIYSTEEKSTAITFRIVNDNLPKYVRNADNYEQIKKLIPEALEEVEKTYPNLTNYFSI | |
| terminal | KNYLKYWSQKGIETYNTVIGEINKQVNLVVQQRKDSKFRKYKMQVLYQQILSDREE | |
| methionine) | QSFVYQQDQEVFAAVNELAELVNGSAFNEAIELLKSPNINENEIFIPYAKLAEVSIKMK | |
| | MGWNGLEEAFINDLQQQYPKKDHEKLVQKLKKEKKVFSLNEIKDVVMKIEHEEDWK | |
| | FVSLLDCVEDYQKQLTETRDAYVEYAKTYAGSTGTSLQGNDVAPIKAFLDSCLQLVR | |
| | WCKLFEYSDLYGNRDKIFYGGAESIILALDSLISVYNKTRNYVTMRPGQARKMHLMF | |
| | NYPEFGDGFSNSKVDSYGTILLREGKKYYLAVIKKGIKVLLEDTINENDSYERLSYML | |
| | FPDVKKMIPKCSISTKKVKEHFENSDDDYTIRKGESYAKELLVKKEDYDLYFVNLYDD | |
| | KKMFQKDYLSKTGDKKGYRQALERWIRFCIRFLQAYKSTKDYDLSELEPISNFRSLD | |
| | EFYDKLDTLLYKIEWKTISREQIKQMESSGQLFLFELYNKDFSEHAKGKKNLFTLYW | |
| | EQIFCEENLKQPVIKLCGGAEMFYRKVAIQKKYVHKKDSILVDKTYVDQNGVRKTLP | |
| | DTIYKEWSDFMNKKITSVSQEASKYKGLVNCHEAKYDITKDKRYTEDQFEFHVPITL | |
| | NYSALGKGQLNDSVLDCLCQKEKYNVIGIDRGERNLLAYCVVNQDGQILEQGTFNKI | |
| | VGGNKQEVDYKQKLQEKEVNRQQARKEWKNIGKIKELKNGYLSQVIYQLTQMMVK | |
| | YDAIVVMEDLNVGFKRGRFKVERQVYQKFEKALIDKLNYLVTKKDENQYGIEGSVSN | |
| | AYQLTEKIKSFKDIGKQNGMIFYVPAGYTSKIDPTTGFVDVLNRTGLTNAKARKAFFE | |
| | NFDDINYSKEDNMFAFSFDYSKFKTFQEMHRKKWTVYTNGKKYIYSKKERKEKQID | |
| | VTELMKEELRKVGITEYDNLYSQITNVEDDKEHADFWKSLQFVFDRTMQLRSSQIDN | |
| | GEDNLEDKIISPVKNAEGVFYESNGNYGDTSQPADADTNGAFHIARKGLLLAENVKK | |
| | TGRGANGKWNSSVKNISNKDWFAFVQK | |
| Wildtype | MERMYEEFRNCYSVRKTLSFKAIPTEETKKHLQLQWEVLGDEIRFENYDKMKMVLD | 74 |
| amino acid | QLHQSYISRKLDNIGEENQKKIVEILEKLVLVMKKIDTTHQKDKEKAQNQLQSLQASL | |
| sequence (with | RKEIGMFFPKNEWQQLQGKNVFKKDGVLSEYNISEENKKNIQCYDGFMTFFKKYNE | |
| N-terminal | TRANIYSTEEKSTAITFRIVNDNLPKYVRNADNYEQIKKLIPEALEEVEKTYPNLTNYF | |
| methionine) | SIKNYLKYWSQKGIETYNTVIGEINKQVNLVVQQRKDSKFRKYKMQVLYQQILSDRE | |
| | EQSFVYQQDQEVFAAVNELAELVNGSAFNEAIELLKSPNINENEIFIPYAKLAEVSIKM | |
| | KMGWNGLEEAFINDLQQQYPKKDHEKLVQKLKKEKKVFSLNEIKDVVMKIEHEEDW | |
| | KFVSLLDCVEDYQKQLTETRDAYVEYAKTYAGSTGTSLQGNDVAPIKAFLDSCLQLV | |
| | RWCKLFEYSDLYGNRDKIFYGGAESIILALDSLISVYNKTRNYVTMRPGQARKMHLM | |
| | FNYPEFGDGFSNSKVDSYGTILLREGKKYYLAVIKKGIKVLLEDTINENDSYERLSYM | |
| | LFPDVKKMIPKCSISTKKVKEHFENSDDDYTIRKGESYAKELLVKKEDYDLYFVNLYD | |
| | DKKMFQKDYLSKTGDKKGYRQALERWIRFCIRFLQAYKSTKDYDLSELEPISNFRSL | |
| | DEFYDKLDTLLYKIEWKTISREQIKQMESSGQLFLFELYNKDFSEHAKGKKNLFTLY | |
| | WEQIFCEENLKQPVIKLCGGAEMFYRKVAIQKKYVHKKDSILVDKTYVDQNGVRKTL | |
| | PDTIYKEWSDFMNKKITSVSQEASKYKGLVNCHEAKYDITKDKRYTEDQFEFHVPIT | |
| | LNYSALGKGQLNDSVLDCLCQKEKYNVIGIDRGERNLLAYCVVNQDGQILEQGTEN | |
| | KIVGGNKQEVDYKQKLQEKEVNRQQARKEWKNIGKIKELKNGYLSQVIYQLTQMMV | |
| | KYDAIVVMEDLNVGFKRGRFKVERQVYQKFEKALIDKLNYLVTKKDENQYGIEGSVS | |
| | NAYQLTEKIKSFKDIGKQNGMIFYVPAGYTSKIDPTTGFVDVLNRTGLTNAKARKAFF | |
| | ENFDDINYSKEDNMFAFSFDYSKFKTFQEMHRKKWTVYTNGKKYIYSKKERKEKQI | |
| | DVTELMKEELRKVGITEYDNLYSQITNVEDDKEHADFWKSLQFVFDRTMQLRSSQID | |
| | NGEDNLEDKIISPVKNAEGVFYESNGNYGDTSQPADADTNGAFHIARKGLLLAENVK | |
| | KTGRGANGKWNSSVKNISNKDWFAFVQK | |
| Expression | MGSGERMYEEFRNCYSVRKTLSFKAIPTEETKKHLQLQWEVLGDEIRFENYDKMK | 75 |
| construct (with | MVLDQLHQSYISRKLDNIGEENQKKIVEILEKLVLVMKKIDTTHQKDKEKAQNQLQSL | |
| N-terminal | QASLRKEIGMFFPKNEWQQLQGKNVFKKDGVLSEYNISEENKKNIQCYDGFMTFFK | |
| methionine, | KYNETRANIYSTEEKSTAITFRIVNDNLPKYVRNADNYEQIKKLIPEALEEVEKTYPNL | |
| V5-tag and C- | TNYFSIKNYLKYWSQKGIETYNTVIGEINKQVNLVVQQRKDSKFRKYKMQVLYQQIL | |
| terminal NLS) | SDREEQSFVYQQDQEVFAAVNELAELVNGSAFNEAIELLKSPNINENEIFIPYAKLAE | |
| aa sequence | VSIKMKMGWNGLEEAFINDLQQQYPKKDHEKLVQKLKKEKKVFSLNEIKDVVMKIEH | |
| | EEDWKFVSLLDCVEDYQKQLTETRDAYVEYAKTYAGSTGTSLQGNDVAPIKAFLDS | |
| | CLQLVRWCKLFEYSDLYGNRDKIFYGGAESIILALDSLISVYNKTRNYVTMRPGQAR | |
| | KMHLMFNYPEFGDGFSNSKVDSYGTILLREGKKYYLAVIKKGIKVLLEDTINENDSYE | |
| | RLSYMLFPDVKKMIPKCSISTKKVKEHFENSDDDYTIRKGESYAKELLVKKEDYDLYF | |
| | VNLYDDKKMFQKDYLSKTGDKKGYRQALERWIRFCIRFLQAYKSTKDYDLSELEPIS | |
| | NFRSLDEFYDKLDTLLYKIEWKTISREQIKQMESSGQLFLFELYNKDFSEHAKGKKNL | |
| | FTLYWEQIFCEENLKQPVIKLCGGAEMFYRKVAIQKKYVHKKDSILVDKTYVDQNGV | |
| | RKTLPDTIYKEWSDFMNKKITSVSQEASKYKGLVNCHEAKYDITKDKRYTEDQFEFH | |
| | VPITLNYSALGKGQLNDSVLDCLCQKEKYNVIGIDRGERNLLAYCVVNQDGQILEQG | |
| | TFNKIVGGNKQEVDYKQKLQEKEVNRQQARKEWKNIGKIKELKNGYLSQVIYQLTQ | |
| | MMVKYDAIVVMEDLNVGFKRGRFKVERQVYQKFEKALIDKLNYLVTKKDENQYGIE | |
| | GSVSNAYQLTEKIKSFKDIGKQNGMIFYVPAGYTSKIDPTTGFVDVLNRTGLTNAKA | |
| | RKAFFENFDDINYSKEDNMFAFSFDYSKFKTFQEMHRKKWTVYTNGKKYIYSKKER | |
| | KEKQIDVTELMKEELRKVGITEYDNLYSQITNVEDDKEHADFWKSLQFVFDRTMQLR | |
| | SSQIDNGEDNLEDKIISPVKNAEGVFYESNGNYGDTSQPADADTNGAFHIARKGLLL | |
| | AENVKKTGRGANGKWNSSVKNISNKDWFAFVQKSRKRTADGSEFESPKKKRKVG | |
| | SGKPIPNPLLGLDST | |
| Wildtype | ATGGAGAGAATGTACGAAGAATTTAGAAATTGTTATTCAGTACGAAAAACATTGT | 76 |
| coding | CATTTAAGGCAATCCCAACAGAGGAAACAAAAAAACATTTACAATTACAATGGGA | |
| sequence (with | AGTGTTGGGGGATGAGATACGTTTTGAAAACTATGATAAAATGAAAATGGTTTTG | |
| N-terminal | GATCAACTTCATCAATCATATATTTCGAGAAAATTAGATAATATAGGAGAAGAAAA | |
| methionine | TCAAAAAAAGATAGTTGAAATCTTAGAGAAACTCGTATTAGTTATGAAAAAGATA | |
| and stop | GATACTACGCATCAAAAGGATAAAGAGAAAGCGCAAAATCAGCTTCAATCGTTA | |
| codon) | CAAGCTTCATTAAGGAAAGAAATAGGAATGTTTTTTCCTAAAAACGAATGGCAAC | |
| | AATTACAGGGAAAAAATGTATTTAAGAAGGATGGGGTACTAAGCGAGTATAACAT | |
| | TTCGGAAGAGAATAAGAAAAATATTCAATGTTATGATGGTTTTATGACATTCTTTA | |
| | AAAAATATAATGAAACTAGAGCAAATATATATAGTACAGAGGAAAAAAGCACGGC | |
| | AATCACTTTTCGAATTGTGAATGATAATCTTCCAAAATATGTGAGAAATGCGGAT | |
| | AATTACGAACAGATTAAAAAATTAATTCCTGAAGCTCTTGAAGAAGTAGAAAAAA | |
| | CATACCCAAATTTGACGAATTATTTCTCGATTAAAAACTATTTGAAGTATTGGAGT | |
| | CAGAAGGGGATTGAAACATACAATACTGTTATTGGAGAAATAAATAAGCAGGTTA | |
| | ATCTTGTAGTACAACAAAGAAAAGATTCGAAATTTAGAAAATACAAGATGCAGGT | |
| | GTTGTATCAACAAATTCTAAGTGATAGAGAGGAACAGTCTTTTGTGTATCAACAG | |
| | GATCAGGAAGTTTTTGCTGCTGTTAATGAACTTGCAGAACTTGTGAACGGTAGT | |
| | GCTTTTAACGAGGCAATTGAATTGTTGAAATCACCTAATATTAACGAAAATGAGA | |
| | TATTTATTCCCTATGCAAAATTAGCAGAAGTATCCATAAAAATGAAAATGGGATG | |
| | GAATGGATTAGAGGAGGCTTTTATAAACGATTTGCAACAGCAGTATCCAAAGAA | |
| | GGATCATGAAAAATTGGTGCAAAAATTAAAAAAAGAGAAAAAAGTTTTTTCTTTGA | |
| | ATGAAATTAAAGATGTTGTTATGAAAATTGAACATGAAGAAGATTGGAAATTTGTT | |
| | AGTTTGCTGGATTGTGTTGAGGATTATCAAAAACAGTTGACAGAGACAAGAGAT | |
| | GCATATGTGGAATATGCAAAAACTTATGCAGGTTCAACCGGTACATCATTACAAG | |
| | GAAATGATGTAGCACCGATAAAAGCATTTTTAGATAGTTGTTTGCAATTGGTACG | |
| | ATGGTGTAAGTTGTTTGAATATTCTGATTTGTATGGAAATCGAGATAAAATATTTT | |
| | ATGGAGGAGCAGAGTCGATTATACTTGCATTAGATTCCTTAATATCTGTGTATAA | |
| | TAAAACAAGAAATTATGTGACTATGCGACCGGGGCAGGCTAGAAAAATGCATTT | |
| | AATGTTTAATTATCCGGAATTCGGTGATGGCTTTAGTAATAGTAAAGTGGATTCT | |
| | TATGGTACGATTTTGCTTCGTGAAGGAAAGAAATATTATTTAGCTGTTATTAAAAA | |
| | AGGCATAAAAGTCTTGCTGGAAGATACCATAAATGAAAATGACAGTTATGAACGT | |
| | TTGAGTTATATGTTGTTTCCTGATGTAAAAAAAATGATACCGAAATGTTCTATTAG | |
| | TACGAAGAAAGTTAAAGAACATTTTGAAAATTCGGATGATGATTATACGATTCGT | |
| | AAAGGTGAATCTTATGCAAAAGAATTACTTGTGAAAAAAGAAGATTATGACCTTT | |
| | ACTTTGTAAATCTTTATGATGATAAGAAGATGTTTCAAAAGGACTATTTGAGTAAA | |
| | ACTGGAGATAAAAAAGGATATAGACAGGCGTTAGAACGCTGGATACGTTTTTGC | |
| | ATTCGATTTTTACAAGCTTATAAGAGTACAAAGGATTATGATCTCAGTGAATTAGA | |
| | GCCAATTTCGAATTTTCGTTCCTTAGATGAGTTTTATGATAAATTGGATACTTTGT | |
| | TATACAAGATAGAGTGGAAAACAATTTCAAGAGAACAAATTAAGCAAATGGAGTC | |
| | ATCTGGTCAGTTGTTTTTATTTGAATTATATAACAAAGATTTCTCTGAACATGCAA | |
| | AAGGAAAGAAAAATTTATTTACATTGTATTGGGAACAGATTTTCTGTGAAGAGAA | |
| | TTTAAAACAGCCAGTGATTAAACTTTGTGGCGGGGCAGAGATGTTTTATCGTAAG | |
| | GTTGCCATTCAAAAAAAATATGTACATAAAAAAGACTCCATTTTGGTGGATAAAA | |
| | CGTATGTGGATCAGAATGGAGTCAGAAAAACACTTCCGGATACTATATATAAAGA | |
| | GTGGTCGGATTTTATGAATAAAAAGATAACATCTGTCAGCCAGGAGGCAAGTAA | |
| | ATATAAAGGTTTGGTTAATTGTCATGAGGCAAAATATGATATTACAAAAGATAAAA | |
| | GATATACGGAAGATCAATTTGAGTTTCATGTGCCAATTACTTTAAATTATTCAGCA | |
| | TTAGGAAAAGGGCAATTAAATGATAGTGTTCTGGATTGTCTATGTCAGAAAGAAA | |
| | AATATAATGTGATAGGAATTGACCGTGGAGAAAGAAACTTGTTGGCTTACTGTGT | |
| | CGTAAATCAAGATGGACAGATTTTAGAACAAGGGACATTTAATAAGATTGTAGGT | |
| | GGAAATAAACAGGAAGTAGATTACAAACAGAAGTTACAGGAGAAAGAAGTAAAT | |
| | CGACAACAAGCAAGAAAAGAGTGGAAAAATATTGGAAAAATTAAAGAATTAAAGA | |
| | ACGGTTATTTGTCTCAGGTTATTTATCAACTGACGCAAATGATGGTAAAATATGA | |
| | TGCTATTGTTGTTATGGAAGATTTGAATGTTGGCTTTAAACGTGGTCGATTTAAG | |
| | GTGGAACGACAGGTTTACCAGAAATTTGAAAAAGCGCTGATTGACAAATTAAATT | |
| | ATTTAGTAACTAAAAAAGATGAAAATCAATATGGAATAGAGGGTAGCGTAAGCAA | |
| | TGCATATCAACTGACAGAAAAAATCAAATCATTTAAAGATATTGGCAAACAAAAC | |
| | GGGATGATATTTTATGTGCCAGCGGGATATACCTCTAAAATAGATCCTACAACAG | |
| | GATTTGTGGATGTGCTAAATCGAACAGGATTAACAAATGCCAAAGCCAGAAAAG | |
| | CGTTCTTTGAAAATTTTGATGATATTAACTATTCAAAAGAAGATAATATGTTTGCC | |
| | TTTTCTTTTGATTATAGCAAGTTTAAGACATTTCAAGAAATGCATAGAAAAAAATG | |
| | GACAGTTTACACAAATGGTAAAAAGTACATTTATTCAAAAAAAGAACGAAAAGAA | |
| | AAACAAATTGATGTTACTGAGTTGATGAAAGAAGAATTGAGAAAAGTAGGAATTA | |
| | CAGAGTATGATAATCTTTATTCGCAAATTACTAATGTGGAAGATGATAAAGAACA | |
| | TGCAGATTTTTGGAAATCTTTACAGTTTGTATTTGATAGAACGATGCAGTTGAGA | |
| | AGTAGTCAAATTGACAATGGAGAGGATAATCTTGAGGATAAGATTATATCTCCGG | |
| | TGAAAAATGCAGAGGGTGTATTTTATGAATCAAATGGAAATTATGGTGACACTTC | |
| | ACAACCTGCAGATGCAGATACAAATGGTGCTTTTCATATTGCAAGGAAGGGATT | |
| | ACTACTTGCAGAAAATGTGAAAAAAACAGGTAGAGGAGCAAATGGAAAATGGAA | |
| | TTCTTCTGTAAAAAATATTTCTAATAAGGATTGGTTTGCATTTGTTCAAAAATAA | |
| Codon | GAACGGATGTACGAGGAGTTCAGAAACTGCTACTCCGTGCGGAAAACACTGTC | 77 |
| optimized | CTTCAAAGCCATCCCTACCGAGGAGACAAAGAAGCACCTGCAGCTGCAGTGGG | |
| coding | AAGTGCTCGGCGACGAGATTAGATTTGAGAATTATGATAAGATGAAAATGGTGC | |
| sequence (no | TGGACCAGCTGCACCAGTCTTACATCAGCCGGAAGCTGGACAACATCGGCGAG | |
| N-terminal | GAGAACCAGAAAAAGATTGTAGAAATCCTGGAGAAGCTGGTGCTGGTGATGAAG | |
| methionine, no | AAGATCGATACAACCCACCAGAAGGACAAGGAGAAGGCCCAGAATCAACTGCA | |
| stop codon) | GAGCCTGCAGGCTTCCCTGCGGAAGGAAATTGGTATGTTTTTCCCAAAGAACGA | |
| | GTGGCAGCAGCTGCAGGGCAAAAACGTGTTCAAGAAGGACGGCGTTCTCAGCG | |
| | AATACAACATCAGCGAGGAAAACAAGAAGAACATCCAGTGTTACGACGGCTTTA | |
| | TGACCTTCTTCAAGAAGTACAACGAGACACGGGCCAATATCTATTCTACGGAGG | |
| | AAAAGAGCACCGCCATCACCTTCAGGATCGTGAATGATAATCTGCCTAAGTATG | |
| | TGCGAAACGCTGACAACTACGAGCAGATAAAGAAGCTGATCCCCGAAGCTCTG | |
| | GAAGAAGTCGAAAAGACCTATCCTAATCTGACCAACTACTTCAGCATCAAGAACT | |
| | ATCTGAAGTACTGGAGCCAGAAGGGGATCGAAACATACAACACCGTGATCGGC | |
| | GAGATCAACAAGCAGGTGAACCTGGTGGTCCAACAGAGAAAGGACAGCAAGTT | |
| | CAGGAAGTACAAAATGCAGGTGCTGTACCAGCAGATCCTATCCGACAGAGAGG | |
| | AGCAGAGCTTCGTGTACCAGCAGGACCAGGAGGTGTTCGCCGCCGTGAACGAG | |
| | CTGGCCGAGCTGGTGAATGGCAGCGCCTTCAATGAAGCTATCGAATTGCTGAAA | |
| | AGCCCAAACATCAACGAGAATGAGATTTTCATCCCCTACGCCAAGCTCGCCGAG | |
| | GTGTCTATCAAGATGAAAATGGGATGGAACGGCCTGGAGGAGGCCTTCATCAA | |
| | CGATCTGCAGCAACAATACCCCAAGAAAGACCACGAAAAATTGGTTCAGAAGCT | |
| | GAAGAAAGAGAAGAAGGTGTTTAGCCTGAATGAAATCAAGGATGTGGTCATGAA | |
| | GATCGAACACGAGGAAGATTGGAAATTCGTGAGCCTGCTGGACTGCGTGGAGG | |
| | ATTACCAGAAGCAGCTTACAGAGACAAGAGATGCCTACGTGGAGTACGCTAAGA | |
| | CATACGCCGGCAGCACAGGCACCAGCCTGCAGGGCAACGACGTGGCCCCTAT | |
| | CAAGGCCTTCCTGGACTCCTGCCTGCAACTGGTGCGGTGGTGCAAGCTGTTCG | |
| | AGTACAGCGACCTGTACGGCAACAGAGACAAGATCTTCTACGGAGGCGCCGAG | |
| | AGCATCATCCTGGCCCTGGATAGCCTGATTTCCGTGTACAACAAAACCAGAAAC | |
| | TACGTGACCATGCGGCCTGGCCAGGCCAGAAAAATGCACCTGATGTTCAACTAC | |
| | CCCGAGTTTGGCGACGGCTTCAGCAACAGCAAAGTGGATTCTTACGGCACCAT | |
| | CCTGCTGAGAGAAGGCAAGAAGTACTACCTGGCTGTGATCAAGAAGGGCATCA | |
| | AAGTGCTGCTGGAGGACACCATTAACGAGAATGACTCTTACGAGCGGCTGTCCT | |
| | ACATGCTGTTCCCCGACGTGAAAAAGATGATCCCTAAGTGCAGCATCAGTACCA | |
| | AGAAGGTGAAAGAGCATTTCGAGAACAGCGACGACGACTACACCATCAGAAAG | |
| | GGCGAGAGCTATGCCAAGGAGCTGCTGGTGAAGAAGGAAGATTACGACCTGTA | |
| | TTTCGTGAACCTGTACGACGACAAAAAGATGTTCCAGAAAGACTACCTGAGCAA | |
| | AACCGGCGACAAGAAGGGATACAGACAGGCCCTGGAGAGGTGGATCAGATTCT | |
| | GCATCAGATTCCTGCAGGCTTACAAGTCTACAAAGGATTATGACCTGTCTGAACT | |
| | GGAACCTATCAGCAACTTCAGAAGCCTGGACGAGTTCTACGATAAGCTGGACAC | |
| | CCTACTGTACAAGATCGAGTGGAAAACCATCTCCAGAGAGCAGATCAAGCAAAT | |
| | GGAATCCTCTGGCCAGCTCTTCCTGTTCGAGTTGTACAACAAGGACTTCTCTGA | |
| | ACACGCCAAGGGAAAGAAGAACCTGTTCACCCTGTACTGGGAGCAAATTTTTTG | |
| | TGAAGAGAACCTGAAGCAGCCTGTGATCAAGCTGTGCGGCGGAGCCGAGATGT | |
| | TCTACAGAAAGGTTGCCATCCAGAAAAAGTACGTGCACAAGAAGGACAGCATCC | |
| | TGGTAGACAAGACCTACGTGGATCAGAACGGCGTTCGCAAGACCCTGCCTGAT | |
| | ACCATCTACAAGGAATGGTCCGACTTCATGAACAAAAAGATCACCAGCGTGTCC | |
| | CAAGAAGCCTCTAAATACAAGGGCCTGGTGAACTGTCACGAGGCCAAGTACGA | |
| | CATCACCAAGGACAAGAGATACACCGAAGATCAATTCGAATTTCACGTGCCAAT | |
| | CACACTGAACTACAGCGCCCTCGGAAAAGGTCAGCTGAACGACAGCGTGCTGG | |
| | ACTGCCTGTGTCAGAAAGAGAAGTACAACGTGATTGGAATCGACCGGGGAGAA | |
| | AGAAACCTGCTGGCCTACTGCGTGGTGAACCAGGATGGCCAGATCCTGGAACA | |
| | GGGCACCTTCAACAAGATCGTGGGCGGCAATAAGCAGGAGGTGGACTATAAGC | |
| | AGAAACTGCAGGAGAAGGAGGTGAATAGACAGCAGGCCAGGAAGGAGTGGAA | |
| | GAACATCGGCAAGATCAAGGAGTTGAAAAACGGCTACCTGAGCCAAGTAATCTA | |
| | CCAGCTGACACAGATGATGGTGAAGTACGATGCCATCGTGGTGATGGAAGATCT | |
| | GAACGTGGGCTTTAAGAGAGGCAGATTCAAGGTTGAGCGGCAGGTGTACCAGA | |
| | AGTTCGAAAAGGCTCTGATCGATAAGCTGAATTATCTGGTCACCAAGAAGGACG | |
| | AGAACCAATACGGGATCGAGGGCAGCGTTTCGAATGCCTACCAGCTGACCGAG | |
| | AAAATCAAGAGCTTCAAAGACATCGGAAAACAGAACGGCATGATCTTCTACGTG | |
| | CCTGCTGGCTATACAAGCAAAATCGACCCTACGACCGGATTCGTCGATGTGCTG | |
| | AACAGAACCGGCCTGACAAACGCCAAGGCTAGAAAAGCCTTCTTCGAGAATTTT | |
| | GACGACATCAACTACTCTAAGGAGGACAACATGTTCGCCTTCAGCTTCGATTAC | |
| | AGCAAGTTCAAGACCTTTCAGGAAATGCATAGAAAAAAGTGGACAGTGTACACA | |
| | AACGGAAAAAAATACATCTACAGCAAGAAGGAACGGAAGGAAAAGCAGATAGAC | |
| | GTGACCGAACTGATGAAAGAAGAGCTGAGAAAGGTGGGCATAACCGAGTACGA | |
| | CAACCTCTACAGCCAGATCACCAACGTGGAAGATGATAAGGAGCACGCCGACTT | |
| | TTGGAAGTCTCTGCAGTTCGTGTTCGACAGAACAATGCAGCTGAGAAGCAGCCA | |
| | GATCGACAACGGCGAGGACAATCTGGAAGATAAGATCATTTCACCTGTGAAAAA | |
| | CGCCGAGGGCGTGTTCTATGAAAGCAACGGCAACTACGGCGATACGAGCCAGC | |
| | CCGCCGACGCGGACACCAACGGCGCCTTCCACATCGCGCGGAAGGGCCTGCT | |
| | GCTCGCCGAGAATGTGAAGAAAACCGGAAGAGGCGCCAATGGCAAATGGAATA | |
| | GCAGCGTGAAGAACATCTCTAACAAGGATTGGTTCGCCTTTGTGCAGAAA | |
| Expression | ATGggctccggaGAACGGATGTACGAGGAGTTCAGAAACTGCTACTCCGTGCGGAA | 78 |
| construct (with | AACACTGTCCTTCAAAGCCATCCCTACCGAGGAGACAAAGAAGCACCTGCAGCT | |
| N-terminal | GCAGTGGGAAGTGCTCGGCGACGAGATTAGATTTGAGAATTATGATAAGATGAA | |
| methionine | AATGGTGCTGGACCAGCTGCACCAGTCTTACATCAGCCGGAAGCTGGACAACA | |
| and stop | TCGGCGAGGAGAACCAGAAAAAGATTGTAGAAATCCTGGAGAAGCTGGTGCTG | |
| codon, | GTGATGAAGAAGATCGATACAACCCACCAGAAGGACAAGGAGAAGGCCCAGAA | |
| includes V5- | TCAACTGCAGAGCCTGCAGGCTTCCCTGCGGAAGGAAATTGGTATGTTTTTCCC | |
| tag and C- | AAAGAACGAGTGGCAGCAGCTGCAGGGCAAAAACGTGTTCAAGAAGGACGGCG | |
| terminal NLS) | TTCTCAGCGAATACAACATCAGCGAGGAAAACAAGAAGAACATCCAGTGTTACG | |
| | ACGGCTTTATGACCTTCTTCAAGAAGTACAACGAGACACGGGCCAATATCTATTC | |
| | TACGGAGGAAAAGAGCACCGCCATCACCTTCAGGATCGTGAATGATAATCTGCC | |
| | TAAGTATGTGCGAAACGCTGACAACTACGAGCAGATAAAGAAGCTGATCCCCGA | |
| | AGCTCTGGAAGAAGTCGAAAAGACCTATCCTAATCTGACCAACTACTTCAGCAT | |
| | CAAGAACTATCTGAAGTACTGGAGCCAGAAGGGGATCGAAACATACAACACCGT | |
| | GATCGGCGAGATCAACAAGCAGGTGAACCTGGTGGTCCAACAGAGAAAGGACA | |
| | GCAAGTTCAGGAAGTACAAAATGCAGGTGCTGTACCAGCAGATCCTATCCGACA | |
| | GAGAGGAGCAGAGCTTCGTGTACCAGCAGGACCAGGAGGTGTTCGCCGCCGT | |
| | GAACGAGCTGGCCGAGCTGGTGAATGGCAGCGCCTTCAATGAAGCTATCGAAT | |
| | TGCTGAAAAGCCCAAACATCAACGAGAATGAGATTTTCATCCCCTACGCCAAGC | |
| | TCGCCGAGGTGTCTATCAAGATGAAAATGGGATGGAACGGCCTGGAGGAGGCC | |
| | TTCATCAACGATCTGCAGCAACAATACCCCAAGAAAGACCACGAAAAATTGGTT | |
| | CAGAAGCTGAAGAAAGAGAAGAAGGTGTTTAGCCTGAATGAAATCAAGGATGTG | |
| | GTCATGAAGATCGAACACGAGGAAGATTGGAAATTCGTGAGCCTGCTGGACTG | |
| | CGTGGAGGATTACCAGAAGCAGCTTACAGAGACAAGAGATGCCTACGTGGAGT | |
| | ACGCTAAGACATACGCCGGCAGCACAGGCACCAGCCTGCAGGGCAACGACGT | |
| | GGCCCCTATCAAGGCCTTCCTGGACTCCTGCCTGCAACTGGTGCGGTGGTGCA | |
| | AGCTGTTCGAGTACAGCGACCTGTACGGCAACAGAGACAAGATCTTCTACGGA | |
| | GGCGCCGAGAGCATCATCCTGGCCCTGGATAGCCTGATTTCCGTGTACAACAA | |
| | AACCAGAAACTACGTGACCATGCGGCCTGGCCAGGCCAGAAAAATGCACCTGA | |
| | TGTTCAACTACCCCGAGTTTGGCGACGGCTTCAGCAACAGCAAAGTGGATTCTT | |
| | ACGGCACCATCCTGCTGAGAGAAGGCAAGAAGTACTACCTGGCTGTGATCAAG | |
| | AAGGGCATCAAAGTGCTGCTGGAGGACACCATTAACGAGAATGACTCTTACGAG | |
| | CGGCTGTCCTACATGCTGTTCCCCGACGTGAAAAAGATGATCCCTAAGTGCAGC | |
| | ATCAGTACCAAGAAGGTGAAAGAGCATTTCGAGAACAGCGACGACGACTACACC | |
| | ATCAGAAAGGGCGAGAGCTATGCCAAGGAGCTGCTGGTGAAGAAGGAAGATTA | |
| | CGACCTGTATTTCGTGAACCTGTACGACGACAAAAAGATGTTCCAGAAAGACTA | |
| | CCTGAGCAAAACCGGCGACAAGAAGGGATACAGACAGGCCCTGGAGAGGTGG | |
| | ATCAGATTCTGCATCAGATTCCTGCAGGCTTACAAGTCTACAAAGGATTATGACC | |
| | TGTCTGAACTGGAACCTATCAGCAACTTCAGAAGCCTGGACGAGTTCTACGATA | |
| | AGCTGGACACCCTACTGTACAAGATCGAGTGGAAAACCATCTCCAGAGAGCAGA | |
| | TCAAGCAAATGGAATCCTCTGGCCAGCTCTTCCTGTTCGAGTTGTACAACAAGG | |
| | ACTTCTCTGAACACGCCAAGGGAAAGAAGAACCTGTTCACCCTGTACTGGGAGC | |
| | AAATTTTTTGTGAAGAGAACCTGAAGCAGCCTGTGATCAAGCTGTGCGGCGGAG | |
| | CCGAGATGTTCTACAGAAAGGTTGCCATCCAGAAAAAGTACGTGCACAAGAAGG | |
| | ACAGCATCCTGGTAGACAAGACCTACGTGGATCAGAACGGCGTTCGCAAGACC | |
| | CTGCCTGATACCATCTACAAGGAATGGTCCGACTTCATGAACAAAAAGATCACC | |
| | AGCGTGTCCCAAGAAGCCTCTAAATACAAGGGCCTGGTGAACTGTCACGAGGC | |
| | CAAGTACGACATCACCAAGGACAAGAGATACACCGAAGATCAATTCGAATTTCA | |
| | CGTGCCAATCACACTGAACTACAGCGCCCTCGGAAAAGGTCAGCTGAACGACA | |
| | GCGTGCTGGACTGCCTGTGTCAGAAAGAGAAGTACAACGTGATTGGAATCGAC | |
| | CGGGGAGAAAGAAACCTGCTGGCCTACTGCGTGGTGAACCAGGATGGCCAGAT | |
| | CCTGGAACAGGGCACCTTCAACAAGATCGTGGGCGGCAATAAGCAGGAGGTGG | |
| | ACTATAAGCAGAAACTGCAGGAGAAGGAGGTGAATAGACAGCAGGCCAGGAAG | |
| | GAGTGGAAGAACATCGGCAAGATCAAGGAGTTGAAAAACGGCTACCTGAGCCA | |
| | AGTAATCTACCAGCTGACACAGATGATGGTGAAGTACGATGCCATCGTGGTGAT | |
| | GGAAGATCTGAACGTGGGCTTTAAGAGAGGCAGATTCAAGGTTGAGCGGCAGG | |
| | TGTACCAGAAGTTCGAAAAGGCTCTGATCGATAAGCTGAATTATCTGGTCACCA | |
| | AGAAGGACGAGAACCAATACGGGATCGAGGGCAGCGTTTCGAATGCCTACCAG | |
| | CTGACCGAGAAAATCAAGAGCTTCAAAGACATCGGAAAACAGAACGGCATGATC | |
| | TTCTACGTGCCTGCTGGCTATACAAGCAAAATCGACCCTACGACCGGATTCGTC | |
| | GATGTGCTGAACAGAACCGGCCTGACAAACGCCAAGGCTAGAAAAGCCTTCTTC | |
| | GAGAATTTTGACGACATCAACTACTCTAAGGAGGACAACATGTTCGCCTTCAGC | |
| | TTCGATTACAGCAAGTTCAAGACCTTTCAGGAAATGCATAGAAAAAAGTGGACA | |
| | GTGTACACAAACGGAAAAAAATACATCTACAGCAAGAAGGAACGGAAGGAAAAG | |
| | CAGATAGACGTGACCGAACTGATGAAAGAAGAGCTGAGAAAGGTGGGCATAAC | |
| | CGAGTACGACAACCTCTACAGCCAGATCACCAACGTGGAAGATGATAAGGAGC | |
| | ACGCCGACTTTTGGAAGTCTCTGCAGTTCGTGTTCGACAGAACAATGCAGCTGA | |
| | GAAGCAGCCAGATCGACAACGGCGAGGACAATCTGGAAGATAAGATCATTTCAC | |
| | CTGTGAAAAACGCCGAGGGCGTGTTCTATGAAAGCAACGGCAACTACGGCGAT | |
| | ACGAGCCAGCCCGCCGACGCGGACACCAACGGCGCCTTCCACATCGCGCGGA | |
| | AGGGCCTGCTGCTCGCCGAGAATGTGAAGAAAACCGGAAGAGGCGCCAATGG | |
| | CAAATGGAATAGCAGCGTGAAGAACATCTCTAACAAGGATTGGTTCGCCTTTGT | |
| | GCAGAAAtctagaAAGCGGACAGCAGACGGCTCCGAATTTGAAAGCCCTAAGAAA | |
| | AAGAGAAAGGTGggatccGGCAAACCTATCCCCAATCCCCTGCTGGGCCTGGACA | |
| | GCACCTGA | |

In some embodiments a ZRGM Type V Cas protein comprises an amino acid sequence of SEQ ID NO: 73, SEQ ID NO:74, or SEQ ID NO:75. In some embodiments, a ZRGM Type V Cas protein has nickase activity, for example resulting from one or more amino acid substitutions relative to the sequence of SEQ ID NO:73, SEQ ID NO:74, or SEQ ID NO:75. In some embodiments, the one or more amino acid substitutions providing nickase activity comprise a D890 substitution, wherein the position of the D890 substitution is defined with respect to the amino acid numbering of SEQ ID NO:74 (corresponding to amino acid 908 of SEQ ID NO: 121). In some embodiments, the one or more amino acid substitutions providing nickase activity comprise an E980 substitution, wherein the position of the E980 substitution is defined with respect to the amino acid numbering of SEQ ID NO:74 (corresponding to amino acid 993 of SEQ ID NO: 121). In some embodiments, the one or more amino acid substitutions providing nickase activity comprise a R1194 substitution, wherein the position of the R1194 substitution is defined with respect to the amino acid numbering of SEQ ID NO:74 (corresponding to amino acid 1226 of SEQ ID NO: 121). In some embodiments, the one or more amino acid substitutions providing nickase activity comprise a D1237 substitution, wherein the position of the D1237 substitution is defined with respect to the amino acid numbering of SEQ ID NO:74 (corresponding to amino acid 1263 of SEQ ID NO: 121). In some embodiments, a ZRGM Type V Cas protein is catalytically inactive, for example due to a R1194 substitution in combination with a D890 substitution, a E980 substitution, and/or D1237 substitution.

### 6.2.14. ZTAE Type V Cas Protein

In one aspect, the disclosure provides ZTAE Type V Cas proteins. ZTAE Type V Cas proteins can be further classified as Type V-A Cas proteins. The ZTAE Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:79. In some embodiments, the ZTAE Type V Cas proteins comprise an amino acid sequence that is at least 95%, at least 96%, at least 97%, at least 98%, or at least 99% identical to SEQ ID NO:79. In some embodiments, a ZTAE Type V Cas protein comprises an amino acid sequence that is identical to SEQ ID NO:79.

Exemplary ZTAE Type V Cas protein sequences and nucleotide sequences encoding exemplary ZTAE Type V Cas proteins are set forth in Table 1N.

TABLE 1N
ZTAE Type V Cas Sequences

| | | SEQ |
|---|---|---|
| | | ID |
| Name | Sequence | NO. |
| Wildtype | SFESFTNVYPVSKTLRFELRPVGATAEKLKESGILEHDTKRGKEYATLKDLLDEQHKE | 79 |
| amino acid | LLADALKPERVKNALKPNSGKSKKDKLVEENYITEDGEIRWETLAAAMEAFRAGEVE | |
| sequence | KNVLEAIQTQFRKLIVTILKADERYPGLTASTPSAVIKTLLKQDVHPEAVETFAKFACYF | |
| (without N- | TGFQENRKNIYAEEKQATAVATRVVHDNFAKFHTQSKIIGVIKNKYPEILQSVEMELM | |
| terminal | DELGGMKITDIFSINSYSKWMTQEGIDFINKIIGGYSPSVGVKVRGLNEFINLYRQQHE | |
| methionine) | EANADRRNLAKMPMLFKQILSDISTRSFIPVMFENDAELKDSIEAFLTGLNDFELNAQK | |
| | FNVVVALGNLFQKIVPCEGIFLDAALMEKVSKTATGDWSLLAQSMEAYAETAFTRAK | |
| | DRDAWLRKNYYSLSELSQVPILKNTDEGMLKFELSAYWSGEKMESFVKGIMDAELA | |
| | MKPVLASIGQKTEEVRLRDRIDDVVKIKGYLDSIQNFLHHLKPFCAPTELNRDADFYS | |
| | DFDALYNQLVLVIPLYNCVRNYVTQKVTEVQKLRLKFDAPTLADGWDANKENDNKAV | |
| | LFEKDGLYFLGILNPNLKAKDRPVFEHESNVTKKSCYRKIVYKLLPGPNKMLPKVFFA | |
| | DSNRTLYHPSKSLLDRYHNGEYKKGDSFDIKFCHELIDYFKASISIHPDWKEFGFQFS | |
| | ATKTYESIDGFYREVEEQGYKVNFAFVRADLIDKYVESGSLFLFQLYNKDFSCASSGK | |
| | PNLHTLYWKSLFAKENLDEPILKLCGGAELFFRPVAIQKPYVHTLGEKLVNRRLGEHG | |
| | KGEAIPERVHKELVDYYNHRVSVLSHDGKAFKDKVVVRDVAHSITKDRRFSEAKFFF | |
| | HVPIMFNRTASKSAKFNDKVVDYLKTTQNVNVIGLDRGERNLIYLTMVNLHGKLIEQR | |
| | SFNLVNGVDYHSKLDLREKERMDARVNWENIGGIKDLKTGYLSAVVHEIAKMMVTNN | |
| | AIVVLEDLNFGFKRGRFKVEKQVYQKFEKMLIDKLNFLMFKECNQAALGGVRRAYQL | |
| | TDKFVSFEKLGKQTGFLFYVPAGYTSKIDPTTGFTNLFNTKKCTNAEGRKVFFEAMN | |
| | SIIYDGSRKSFAFSFDYGNPVFRASQTSFKKEWTVYSADTRIVYNRGEKTVNTIHPTQI | |
| | LHDALCALGIDVHDGLNVLNVVRETPADKIHAKFFSDLFYAFDRTLQMRNSVSGTDE | |
| | DYIQSPVLNATGEFFDSRKADSTLPQDADANGAYHIALKGLLLLQRMKDIGSDIKLDLS | |
| | IKHEDWFAFAQKRCQR | |
| Wildtype | MSFESFTNVYPVSKTLRFELRPVGATAEKLKESGILEHDTKRGKEYATLKDLLDEQHK | 80 |
| amino acid | ELLADALKPERVKNALKPNSGKSKKDKLVEENYITEDGEIRWETLAAAMEAFRAGEV | |
| sequence (with | EKNVLEAIQTQFRKLIVTILKADERYPGLTASTPSAVIKTLLKQDVHPEAVETFAKFACY | |
| N-terminal | FTGFQENRKNIYAEEKQATAVATRVVHDNFAKFHTQSKIIGVIKNKYPEILQSVEMELM | |
| methionine) | DELGGMKITDIFSINSYSKWMTQEGIDFINKIIGGYSPSVGVKVRGLNEFINLYRQQHE | |
| | EANADRRNLAKMPMLFKQILSDISTRSFIPVMFENDAELKDSIEAFLTGLNDFELNAQK | |
| | FNVVVALGNLFQKIVPCEGIFLDAALMEKVSKTATGDWSLLAQSMEAYAETAFTRAK | |
| | DRDAWLRKNYYSLSELSQVPILKNTDEGMLKFELSAYWSGEKMESFVKGIMDAELA | |
| | MKPVLASIGQKTEEVRLRDRIDDVVKIKGYLDSIQNFLHHLKPFCAPTELNRDADFYS | |
| | DFDALYNQLVLVIPLYNCVRNYVTQKVTEVQKLRLKFDAPTLADGWDANKENDNKAV | |
| | LFEKDGLYFLGILNPNLKAKDRPVFEHESNVTKKSCYRKIVYKLLPGPNKMLPKVFFA | |
| | DSNRTLYHPSKSLLDRYHNGEYKKGDSFDIKFCHELIDYFKASISIHPDWKEFGFQFS | |
| | ATKTYESIDGFYREVEEQGYKVNFAFVRADLIDKYVESGSLFLFQLYNKDFSCASSGK | |
| | PNLHTLYWKSLFAKENLDEPILKLCGGAELFFRPVAIQKPYVHTLGEKLVNRRLGEHG | |
| | KGEAIPERVHKELVDYYNHRVSVLSHDGKAFKDKVVVRDVAHSITKDRRFSEAKFFF | |
| | HVPIMFNRTASKSAKFNDKVVDYLKTTQNVNVIGLDRGERNLIYLTMVNLHGKLIEQR | |
| | SFNLVNGVDYHSKLDLREKERMDARVNWENIGGIKDLKTGYLSAVVHEIAKMMVTNN | |
| | AIVVLEDLNFGFKRGRFKVEKQVYQKFEKMLIDKLNFLMFKECNQAALGGVRRAYQL | |
| | TDKFVSFEKLGKQTGFLFYVPAGYTSKIDPTTGFTNLFNTKKCTNAEGRKVFFEAMN | |
| | SIIYDGSRKSFAFSFDYGNPVFRASQTSFKKEWTVYSADTRIVYNRGEKTVNTIHPTQI | |
| | LHDALCALGIDVHDGLNVLNVVRETPADKIHAKFFSDLFYAFDRTLQMRNSVSGTDE | |
| | DYIQSPVLNATGEFFDSRKADSTLPQDADANGAYHIALKGLLLLQRMKDIGSDIKLDLS | |
| | IKHEDWFAFAQKRCQR | |
| Expression | MGSGSFESFTNVYPVSKTLRFELRPVGATAEKLKESGILEHDTKRGKEYATLKDLLDE | 81 |
| construct (with | QHKELLADALKPERVKNALKPNSGKSKKDKLVEENYITEDGEIRWETLAAAMEAFRA | |
| N-terminal | GEVEKNVLEAIQTQFRKLIVTILKADERYPGLTASTPSAVIKTLLKQDVHPEAVETFAKF | |
| methionine, | ACYFTGFQENRKNIYAEEKQATAVATRVVHDNFAKFHTQSKIIGVIKNKYPEILQSVEM | |
| V5-tag and C- | ELMDELGGMKITDIFSINSYSKWMTQEGIDFINKIIGGYSPSVGVKVRGLNEFINLYRQ | |
| terminal NLS) | QHEEANADRRNLAKMPMLFKQILSDISTRSFIPVMFENDAELKDSIEAFLTGLNDFELN | |
| aa sequence | AQKFNVVVALGNLFQKIVPCEGIFLDAALMEKVSKTATGDWSLLAQSMEAYAETAFT | |
| | RAKDRDAWLRKNYYSLSELSQVPILKNTDEGMLKFELSAYWSGEKMESFVKGIMDA | |
| | ELAMKPVLASIGQKTEEVRLRDRIDDVVKIKGYLDSIQNFLHHLKPFCAPTELNRDADF | |
| | YSDFDALYNQLVLVIPLYNCVRNYVTQKVTEVQKLRLKFDAPTLADGWDANKENDNK | |
| | AVLFEKDGLYFLGILNPNLKAKDRPVFEHESNVTKKSCYRKIVYKLLPGPNKMLPKVF | |
| | FADSNRTLYHPSKSLLDRYHNGEYKKGDSFDIKFCHELIDYFKASISIHPDWKEFGFQ | |
| | FSATKTYESIDGFYREVEEQGYKVNFAFVRADLIDKYVESGSLFLFQLYNKDFSCASS | |
| | GKPNLHTLYWKSLFAKENLDEPILKLCGGAELFFRPVAIQKPYVHTLGEKLVNRRLGE | |
| | HGKGEAIPERVHKELVDYYNHRVSVLSHDGKAFKDKVVVRDVAHSITKDRRFSEAKF | |
| | FFHVPIMFNRTASKSAKFNDKVVDYLKTTQNVNVIGLDRGERNLIYLTMVNLHGKLIE | |
| | QRSFNLVNGVDYHSKLDLREKERMDARVNWENIGGIKDLKTGYLSAVVHEIAKMMVT | |
| | NNAIVVLEDLNFGFKRGRFKVEKQVYQKFEKMLIDKLNFLMFKECNQAALGGVRRAY | |
| | QLTDKFVSFEKLGKQTGFLFYVPAGYTSKIDPTTGFTNLFNTKKCTNAEGRKVFFEAM | |
| | NSIIYDGSRKSFAFSFDYGNPVFRASQTSFKKEWTVYSADTRIVYNRGEKTVNTIHPT | |
| | QILHDALCALGIDVHDGLNVLNVVRETPADKIHAKFFSDLFYAFDRTLQMRNSVSGTD | |
| | EDYIQSPVLNATGEFFDSRKADSTLPQDADANGAYHIALKGLLLLQRMKDIGSDIKLDL | |
| | SIKHEDWFAFAQKRCQRSRKRTADGSEFESPKKKRKVGSGKPIPNPLLGLDST | |
| Wildtype | ATGAGTTTTGAATCATTCACTAACGTTTATCCCGTTTCCAAGACTTTGCGCTTTGA | 82 |
| coding | GCTGAGGCCCGTTGGTGCAACTGCAGAGAAGCTTAAGGAAAGTGGTATCCTTGA | |
| sequence (with | GCATGATACGAAACGAGGTAAGGAATATGCGACTCTCAAGGATCTGCTTGATGAG | |
| N-terminal | CAACATAAGGAGTTACTTGCTGACGCCCTAAAACCTGAACGTGTGAAGAATGCGC | |
| methionine | TTAAGCCCAATAGTGGTAAGAGTAAAAAAGATAAATTGGTTGAAGAGAATTACATT | |
| and stop | ACGGAAGACGGGGAGATTCGATGGGAAACTCTTGCGGCTGCGATGGAGGCATTT | |
| codon) | CGCGCCGGTGAGGTAGAGAAAAATGTGCTTGAAGCAATACAGACGCAATTTAGA | |
| | AAGCTGATTGTAACGATACTGAAGGCGGATGAGCGGTATCCGGGACTGACAGCT | |
| | TCAACGCCTTCGGCTGTCATTAAGACTCTTCTTAAGCAGGATGTTCATCCAGAAG | |
| | CAGTAGAGACATTTGCAAAATTTGCCTGTTATTTTACCGGTTTTCAGGAAAATCGG | |
| | AAGAATATCTATGCGGAAGAAAAGCAAGCAACTGCAGTTGCAACGCGAGTTGTTC | |
| | ATGATAATTTCGCAAAGTTCCATACACAATCGAAAATAATAGGTGTCATAAAGAAT | |
| | AAATATCCAGAAATCCTTCAGTCGGTAGAAATGGAATTGATGGACGAATTAGGTG | |
| | GGATGAAAATCACTGATATCTTTTCTATCAACAGCTATTCCAAATGGATGACGCAA | |
| | GAAGGGATAGACTTTATTAATAAGATTATAGGTGGCTATAGCCCATCTGTTGGTGT | |
| | GAAGGTGCGTGGTCTGAACGAGTTCATTAATCTTTATCGGCAGCAGCATGAAGAG | |
| | GCAAATGCAGATCGGCGGAATCTCGCAAAAATGCCGATGCTGTTTAAACAAATTT | |
| | TAAGTGATATTTCGACACGATCATTCATTCCGGTGATGTTTGAAAATGATGCGGAA | |
| | CTAAAGGATTCAATAGAAGCATTCTTGACAGGTCTGAATGATTTTGAGTTGAATGC | |
| | TCAGAAGTTTAACGTTGTCGTTGCATTAGGTAATCTTTTCCAAAAAATTGTGCCTT | |
| | GCGAAGGTATTTTCTTGGATGCAGCATTGATGGAAAAAGTTTCGAAGACGGCTAC | |
| | AGGAGATTGGAGTCTTCTTGCTCAGTCGATGGAGGCGTATGCAGAGACAGCATT | |
| | CACAAGAGCAAAAGACCGAGACGCATGGCTAAGGAAAAATTATTATTCGCTGTCC | |
| | GAGCTGAGCCAAGTTCCGATTTTGAAGAACACTGATGAAGGAATGTTGAAGTTTG | |
| | AACTATCTGCCTATTGGTCAGGCGAAAAGATGGAAAGTTTTGTTAAAGGAATCAT | |
| | GGATGCTGAATTGGCAATGAAACCAGTTCTTGCCAGCATTGGTCAGAAAACCGAA | |
| | GAGGTGCGTCTTCGTGATCGGATTGACGATGTCGTAAAAATCAAGGGATATCTTG | |
| | ATTCAATTCAGAATTTTTTACATCACCTAAAACCGTTTTGTGCTCCAACTGAATTGA | |
| | ATCGTGATGCGGATTTTTATTCTGACTTTGACGCATTGTATAATCAGCTTGTACTG | |
| | GTTATACCGCTTTATAACTGTGTCCGCAATTACGTGACACAGAAAGTGACAGAGG | |
| | TTCAGAAACTGAGGCTAAAGTTTGATGCCCCTACATTGGCGGACGGATGGGACG | |
| | CGAATAAAGAAAATGATAATAAGGCAGTTCTGTTTGAAAAGGACGGGCTATATTTT | |
| | CTTGGAATCCTGAATCCTAACCTGAAGGCGAAAGATCGTCCAGTCTTTGAGCATG | |
| | AAAGTAATGTTACAAAGAAATCTTGTTATCGCAAGATTGTCTATAAACTTTTGCCA | |
| | GGACCAAATAAAATGCTTCCCAAGGTCTTTTTTGCTGATTCCAATAGGACACTGTA | |
| | CCATCCTTCCAAGTCGTTGCTGGATCGTTATCACAACGGTGAATACAAGAAAGGC | |
| | GATTCATTCGACATCAAATTCTGTCATGAATTGATTGATTATTTTAAAGCCTCGATT | |
| | AGTATTCACCCCGATTGGAAGGAATTCGGTTTCCAATTCAGTGCGACAAAAACAT | |
| | ATGAGAGCATTGATGGTTTTTATCGTGAGGTTGAGGAGCAAGGATATAAAGTTAA | |
| | TTTTGCTTTTGTAAGGGCGGATTTAATTGACAAATATGTGGAAAGTGGAAGTTTGT | |
| | TCCTTTTCCAATTGTATAACAAGGATTTCTCTTGTGCGTCATCTGGGAAGCCAAAC | |
| | CTCCACACGCTTTACTGGAAGAGCCTCTTTGCAAAAGAAAACCTTGATGAGCCGA | |
| | TTCTGAAGTTGTGTGGGGGTGCAGAGCTATTCTTCCGCCCAGTTGCAATCCAGAA | |
| | GCCGTATGTACATACCTTGGGAGAAAAGTTGGTCAATCGCAGGCTTGGCGAGCA | |
| | CGGTAAGGGAGAGGCAATCCCGGAGAGAGTTCACAAGGAACTCGTGGACTACTA | |
| | CAACCATCGTGTGTCGGTGCTGAGTCATGATGGGAAGGCATTTAAAGACAAGGTT | |
| | GTTGTTCGGGATGTCGCACATTCGATTACAAAAGATCGTCGATTCTCAGAGGCAA | |
| | AGTTTTTTTTCCATGTTCCGATCATGTTTAACCGTACAGCATCGAAGAGTGCAAAG | |
| | TTTAACGACAAAGTTGTGGACTATCTCAAGACCACTCAGAATGTAAACGTTATCG | |
| | GGTTGGATCGAGGAGAAAGAAATCTGATTTATCTGACAATGGTAAATTTGCACGG | |
| | AAAGCTGATAGAGCAGCGTAGTTTCAACCTAGTTAATGGTGTGGATTATCATTCAA | |
| | AGCTAGATTTGCGAGAAAAGGAGCGCATGGACGCACGCGTTAATTGGGAGAACA | |
| | TTGGGGGAATTAAAGATCTTAAGACCGGATATCTTTCCGCGGTTGTTCATGAGAT | |
| | TGCGAAGATGATGGTGACGAATAATGCCATTGTTGTCTTGGAGGACTTGAACTTC | |
| | GGTTTCAAACGTGGGCGGTTCAAGGTTGAGAAACAGGTCTATCAGAAGTTTGAGA | |
| | AGATGCTGATTGATAAACTGAATTTCCTGATGTTCAAGGAATGCAATCAAGCGGC | |
| | TCTCGGTGGTGTTCGCCGTGCATATCAATTGACGGATAAATTCGTGAGTTTTGAA | |
| | AAACTTGGTAAACAAACGGGTTTCCTGTTTTATGTTCCGGCGGGCTACACATCGA | |
| | AGATTGATCCAACAACTGGATTCACCAACCTCTTCAACACGAAAAAATGCACTAAT | |
| | GCCGAAGGTCGGAAGGTCTTCTTTGAGGCGATGAACTCTATCATATATGACGGAT | |
| | CAAGGAAGTCGTTTGCGTTCTCATTTGATTACGGCAACCCAGTTTTTAGAGCAAG | |
| | TCAAACGAGTTTTAAAAAAGAATGGACCGTCTATTCCGCTGATACGCGCATTGTC | |
| | TACAATCGTGGCGAGAAAACTGTTAATACGATCCATCCGACACAAATTCTTCATGA | |
| | TGCTTTGTGTGCACTCGGCATTGACGTTCATGACGGATTGAACGTCTTGAACGTA | |
| | GTTCGTGAGACGCCAGCGGACAAGATTCATGCTAAGTTTTTCTCAGACTTGTTCT | |
| | ATGCGTTTGATCGTACACTTCAGATGCGTAACAGTGTTTCAGGAACAGATGAAGA | |
| | CTATATCCAATCGCCTGTTTTGAATGCGACAGGTGAGTTTTTTGATTCGCGGAAA | |
| | GCAGACAGTACTCTTCCGCAGGATGCCGATGCCAATGGTGCCTACCACATCGCA | |
| | TTAAAGGGACTTTTGCTGCTACAACGCATGAAAGATATTGGCAGTGATATCAAGC | |
| | TTGATCTATCCATTAAGCATGAGGACTGGTTTGCGTTTGCACAAAAGCGTTGCCA | |
| | GAGATAA | |
| Codon | AGCTTCGAGTCTTTCACTAACGTATACCCTGTGTCTAAAACCCTGCGTTTTGAACT | 83 |
| optimized | GCGGCCTGTGGGCGCCACTGCCGAGAAGCTGAAGGAGAGCGGCATCCTGGAGC | |
| coding | ACGATACCAAGCGGGGCAAGGAATACGCTACACTGAAGGACCTGCTGGACGAG | |
| sequence (no | CAGCACAAAGAGCTACTGGCCGACGCCCTGAAGCCAGAGAGAGTGAAGAACGC | |
| N-terminal | CCTGAAGCCCAACAGCGGCAAGTCCAAAAAGGACAAGCTGGTCGAAGAGAACTA | |
| methionine, no | CATTACAGAAGATGGAGAGATCAGATGGGAGACACTGGCCGCTGCTATGGAGGC | |
| stop codon) | CTTCAGAGCTGGCGAAGTGGAGAAGAACGTGCTGGAAGCGATCCAGACACAGTT | |
| | TCGGAAGCTGATCGTGACCATCCTGAAAGCCGACGAGAGATACCCTGGACTGAC | |
| | CGCCTCTACACCTAGCGCCGTCATCAAGACCTTGCTGAAGCAGGACGTGCACCC | |
| | CGAGGCCGTAGAGACATTCGCTAAATTTGCCTGTTACTTCACCGGCTTTCAGGAA | |
| | AACAGAAAGAATATCTACGCCGAAGAAAAACAGGCCACCGCCGTGGCCACACGG | |
| | GTTGTCCACGACAACTTCGCCAAATTTCACACCCAGTCTAAGATTATCGGCGTGA | |
| | TCAAAAACAAGTACCCCGAGATCCTGCAGAGCGTCGAGATGGAACTGATGGACG | |
| | AACTTGGGGGAATGAAGATCACCGATATCTTCAGTATCAACAGCTACAGCAAGTG | |
| | GATGACCCAGGAGGGAATCGACTTCATCAACAAAATCATCGGCGGCTACAGCCC | |
| | TAGCGTGGGCGTCAAAGTGAGAGGCCTGAACGAGTTCATCAACCTGTACAGACA | |
| | GCAGCACGAGGAAGCCAACGCCGACCGGCGGAACCTGGCTAAGATGCCTATGC | |
| | TGTTTAAACAAATTCTGAGCGACATCAGCACCCGGAGCTTCATCCCTGTGATGTT | |
| | CGAGAATGACGCCGAGCTCAAGGACAGCATCGAGGCCTTCCTGACAGGCCTGAA | |
| | TGATTTCGAGCTGAACGCTCAGAAGTTCAACGTTGTGGTGGCCCTGGGGAACCT | |
| | GTTTCAGAAGATTGTGCCTTGTGAAGGCATCTTCCTGGACGCTGCCCTGATGGAA | |
| | AAGGTTTCCAAGACAGCTACAGGCGACTGGAGCCTGCTCGCACAGTCTATGGAA | |
| | GCCTACGCCGAAACAGCCTTTACAAGAGCCAAGGACCGGGACGCCTGGCTGAG | |
| | AAAGAATTACTACAGCCTGTCCGAGCTGAGCCAGGTGCCAATCCTGAAGAACACT | |
| | GATGAGGGCATGCTGAAGTTCGAGCTGAGCGCCTACTGGTCCGGCGAGAAAATG | |
| | GAATCTTTCGTGAAGGGCATCATGGACGCCGAGCTGGCCATGAAGCCAGTGCTG | |
| | GCCAGCATCGGCCAGAAAACCGAAGAGGTGCGGCTGAGAGATAGAATCGACGA | |
| | CGTGGTGAAGATCAAGGGCTACCTGGACAGCATCCAGAATTTCCTGCACCACCT | |
| | GAAGCCTTTCTGTGCCCCTACCGAGCTGAACCGGGACGCCGACTTCTACTCTGA | |
| | CTTCGATGCTCTGTACAATCAACTGGTGCTGGTGATTCCCCTGTACAACTGCGTG | |
| | AGAAACTACGTCACCCAAAAGGTTACCGAGGTGCAGAAGCTGCGCCTCAAGTTC | |
| | GATGCACCTACCCTGGCCGATGGATGGGACGCCAATAAAGAGAATGACAACAAA | |
| | GCCGTCCTGTTCGAGAAAGACGGCCTGTATTTCCTCGGCATCCTCAACCCTAACC | |
| | TGAAAGCCAAGGACCGGCCTGTGTTCGAACATGAAAGCAACGTGACCAAGAAGT | |
| | CATGCTACCGGAAGATTGTGTACAAACTGCTGCCAGGCCCTAACAAGATGCTGC | |
| | CTAAGGTGTTCTTTGCCGATAGCAACAGGACACTGTACCACCCTAGCAAGAGCCT | |
| | GCTGGACCGGTATCACAACGGCGAGTACAAGAAGGGCGATAGCTTTGATATCAA | |
| | GTTTTGCCACGAGCTGATCGACTACTTCAAGGCCTCTATCTCTATTCACCCTGAC | |
| | TGGAAGGAGTTCGGCTTTCAATTTTCTGCCACAAAGACCTACGAGTCTATCGACG | |
| | GCTTCTATAGAGAGGTGGAAGAGCAGGGCTACAAGGTGAACTTCGCCTTTGTGC | |
| | GTGCTGACCTGATCGATAAGTACGTGGAAAGCGGCTCCCTGTTCCTGTTCCAGC | |
| | TCTATAACAAGGACTTCAGCTGTGCCTCTAGCGGCAAGCCGAATCTTCATACACT | |
| | GTACTGGAAAAGCCTGTTCGCCAAGGAGAACCTGGACGAGCCTATACTGAAGCT | |
| | GTGCGGCGGCGCCGAGCTGTTCTTCAGACCCGTGGCGATCCAGAAACCCTACG | |
| | TGCACACATTGGGCGAAAAGCTGGTGAATAGACGGCTCGGCGAGCACGGCAAG | |
| | GGCGAGGCTATCCCTGAGCGGGTGCACAAGGAACTGGTGGACTACTACAACCAC | |
| | AGAGTGAGCGTGCTCAGTCACGATGGAAAGGCCTTCAAGGACAAGGTGGTGGTT | |
| | CGGGACGTGGCCCACAGCATCACCAAGGACCGACGGTTTAGCGAGGCCAAGTT | |
| | CTTCTTCCACGTGCCCATCATGTTTAACCGGACCGCCAGCAAGAGCGCCAAGTT | |
| | CAACGACAAGGTGGTGGACTACCTGAAAACCACCCAAAACGTGAACGTGATCGG | |
| | ACTGGACAGAGGTGAAAGAAACCTGATCTACCTCACAATGGTGAACCTGCATGG | |
| | CAAGCTCATCGAGCAGCGGAGCTTCAACCTGGTGAATGGCGTGGACTACCATTC | |
| | TAAGCTGGATCTGCGCGAGAAGGAACGTATGGATGCTAGAGTGAACTGGGAGAA | |
| | TATCGGCGGCATAAAGGATCTGAAAACCGGCTACCTGAGCGCCGTGGTGCACGA | |
| | GATCGCCAAAATGATGGTGACAAACAACGCCATCGTGGTGCTGGAAGATCTGAA | |
| | CTTTGGATTCAAGAGAGGCAGATTCAAAGTGGAAAAGCAGGTGTACCAGAAATTC | |
| | GAGAAGATGCTGATCGACAAACTGAACTTCCTGATGTTCAAAGAGTGCAACCAGG | |
| | CCGCCCTGGGCGGCGTGCGGCGGGCCTATCAGCTGACCGACAAGTTCGTGAGC | |
| | TTCGAGAAGCTGGGAAAGCAGACCGGCTTCCTGTTCTATGTGCCCGCCGGCTAT | |
| | ACAAGCAAAATCGATCCTACAACCGGTTTCACCAACCTGTTCAATACCAAGAAAT | |
| | GCACCAACGCCGAGGGAAGAAAGGTGTTCTTCGAGGCTATGAACAGCATCATCT | |
| | ACGACGGCTCCAGAAAATCTTTCGCCTTTAGCTTCGACTACGGCAACCCCGTGTT | |
| | TCGAGCCTCCCAGACCAGCTTCAAGAAGGAATGGACCGTGTACAGCGCCGATAC | |
| | AAGAATCGTGTATAATCGGGGCGAAAAGACCGTAAACACCATCCACCCTACCCA | |
| | GATCCTGCACGACGCCCTGTGCGCCTTGGGAATCGACGTGCACGATGGGTTAAA | |
| | TGTCTTGAACGTCGTGAGAGAGACACCCGCTGATAAGATCCACGCCAAGTTCTTC | |
| | AGCGATCTCTTCTACGCCTTCGACAGAACCCTGCAGATGAGGAACTCTGTGAGC | |
| | GGGACCGACGAAGATTACATCCAGAGCCCTGTGCTGAATGCTACCGGCGAGTTC | |
| | TTTGACAGCAGAAAAGCCGACAGCACCCTGCCCCAGGACGCAGACGCTAATGGA | |
| | GCCTACCACATCGCCCTGAAGGGCCTGCTGCTCCTGCAGAGAATGAAGGATATC | |
| | GGCTCAGATATCAAGCTGGATCTGTCTATTAAGCACGAGGATTGGTTCGCCTTCG | |
| | CTCAGAAGCGGTGCCAGAGA | |
| Expression | ATGggctccggaAGCTTCGAGTCTTTCACTAACGTATACCCTGTGTCTAAAACCCTGC | 84 |
| construct (with | GTTTTGAACTGCGGCCTGTGGGCGCCACTGCCGAGAAGCTGAAGGAGAGCGGC | |
| N-terminal | ATCCTGGAGCACGATACCAAGCGGGGCAAGGAATACGCTACACTGAAGGACCTG | |
| methionine | CTGGACGAGCAGCACAAAGAGCTACTGGCCGACGCCCTGAAGCCAGAGAGAGT | |
| and stop | GAAGAACGCCCTGAAGCCCAACAGCGGCAAGTCCAAAAAGGACAAGCTGGTCGA | |
| codon, | AGAGAACTACATTACAGAAGATGGAGAGATCAGATGGGAGACACTGGCCGCTGC | |
| includes V5- | TATGGAGGCCTTCAGAGCTGGCGAAGTGGAGAAGAACGTGCTGGAAGCGATCCA | |
| tag and C- | GACACAGTTTCGGAAGCTGATCGTGACCATCCTGAAAGCCGACGAGAGATACCC | |
| terminal NLS) | TGGACTGACCGCCTCTACACCTAGCGCCGTCATCAAGACCTTGCTGAAGCAGGA | |
| | CGTGCACCCCGAGGCCGTAGAGACATTCGCTAAATTTGCCTGTTACTTCACCGG | |
| | CTTTCAGGAAAACAGAAAGAATATCTACGCCGAAGAAAAACAGGCCACCGCCGT | |
| | GGCCACACGGGTTGTCCACGACAACTTCGCCAAATTTCACACCCAGTCTAAGATT | |
| | ATCGGCGTGATCAAAAACAAGTACCCCGAGATCCTGCAGAGCGTCGAGATGGAA | |
| | CTGATGGACGAACTTGGGGGAATGAAGATCACCGATATCTTCAGTATCAACAGCT | |
| | ACAGCAAGTGGATGACCCAGGAGGGAATCGACTTCATCAACAAAATCATCGGCG | |
| | GCTACAGCCCTAGCGTGGGCGTCAAAGTGAGAGGCCTGAACGAGTTCATCAACC | |
| | TGTACAGACAGCAGCACGAGGAAGCCAACGCCGACCGGCGGAACCTGGCTAAG | |
| | ATGCCTATGCTGTTTAAACAAATTCTGAGCGACATCAGCACCCGGAGCTTCATCC | |
| | CTGTGATGTTCGAGAATGACGCCGAGCTCAAGGACAGCATCGAGGCCTTCCTGA | |
| | CAGGCCTGAATGATTTCGAGCTGAACGCTCAGAAGTTCAACGTTGTGGTGGCCC | |
| | TGGGGAACCTGTTTCAGAAGATTGTGCCTTGTGAAGGCATCTTCCTGGACGCTG | |
| | CCCTGATGGAAAAGGTTTCCAAGACAGCTACAGGCGACTGGAGCCTGCTCGCAC | |
| | AGTCTATGGAAGCCTACGCCGAAACAGCCTTTACAAGAGCCAAGGACCGGGACG | |
| | CCTGGCTGAGAAAGAATTACTACAGCCTGTCCGAGCTGAGCCAGGTGCCAATCC | |
| | TGAAGAACACTGATGAGGGCATGCTGAAGTTCGAGCTGAGCGCCTACTGGTCCG | |
| | GCGAGAAAATGGAATCTTTCGTGAAGGGCATCATGGACGCCGAGCTGGCCATGA | |
| | AGCCAGTGCTGGCCAGCATCGGCCAGAAAACCGAAGAGGTGCGGCTGAGAGAT | |
| | AGAATCGACGACGTGGTGAAGATCAAGGGCTACCTGGACAGCATCCAGAATTTC | |
| | CTGCACCACCTGAAGCCTTTCTGTGCCCCTACCGAGCTGAACCGGGACGCCGAC | |
| | TTCTACTCTGACTTCGATGCTCTGTACAATCAACTGGTGCTGGTGATTCCCCTGTA | |
| | CAACTGCGTGAGAAACTACGTCACCCAAAAGGTTACCGAGGTGCAGAAGCTGCG | |
| | CCTCAAGTTCGATGCACCTACCCTGGCCGATGGATGGGACGCCAATAAAGAGAA | |
| | TGACAACAAAGCCGTCCTGTTCGAGAAAGACGGCCTGTATTTCCTCGGCATCCTC | |
| | AACCCTAACCTGAAAGCCAAGGACCGGCCTGTGTTCGAACATGAAAGCAACGTG | |
| | ACCAAGAAGTCATGCTACCGGAAGATTGTGTACAAACTGCTGCCAGGCCCTAACA | |
| | AGATGCTGCCTAAGGTGTTCTTTGCCGATAGCAACAGGACACTGTACCACCCTAG | |
| | CAAGAGCCTGCTGGACCGGTATCACAACGGCGAGTACAAGAAGGGCGATAGCTT | |
| | TGATATCAAGTTTTGCCACGAGCTGATCGACTACTTCAAGGCCTCTATCTCTATTC | |
| | ACCCTGACTGGAAGGAGTTCGGCTTTCAATTTTCTGCCACAAAGACCTACGAGTC | |
| | TATCGACGGCTTCTATAGAGAGGTGGAAGAGCAGGGCTACAAGGTGAACTTCGC | |
| | CTTTGTGCGTGCTGACCTGATCGATAAGTACGTGGAAAGCGGCTCCCTGTTCCT | |
| | GTTCCAGCTCTATAACAAGGACTTCAGCTGTGCCTCTAGCGGCAAGCCGAATCTT | |
| | CATACACTGTACTGGAAAAGCCTGTTCGCCAAGGAGAACCTGGACGAGCCTATA | |
| | CTGAAGCTGTGCGGCGGCGCCGAGCTGTTCTTCAGACCCGTGGCGATCCAGAA | |
| | ACCCTACGTGCACACATTGGGCGAAAAGCTGGTGAATAGACGGCTCGGCGAGCA | |
| | CGGCAAGGGCGAGGCTATCCCTGAGCGGGTGCACAAGGAACTGGTGGACTACT | |
| | ACAACCACAGAGTGAGCGTGCTCAGTCACGATGGAAAGGCCTTCAAGGACAAGG | |
| | TGGTGGTTCGGGACGTGGCCCACAGCATCACCAAGGACCGACGGTTTAGCGAG | |
| | GCCAAGTTCTTCTTCCACGTGCCCATCATGTTTAACCGGACCGCCAGCAAGAGC | |
| | GCCAAGTTCAACGACAAGGTGGTGGACTACCTGAAAACCACCCAAAACGTGAAC | |
| | GTGATCGGACTGGACAGAGGTGAAAGAAACCTGATCTACCTCACAATGGTGAAC | |
| | CTGCATGGCAAGCTCATCGAGCAGCGGAGCTTCAACCTGGTGAATGGCGTGGAC | |
| | TACCATTCTAAGCTGGATCTGCGCGAGAAGGAACGTATGGATGCTAGAGTGAACT | |
| | GGGAGAATATCGGCGGCATAAAGGATCTGAAAACCGGCTACCTGAGCGCCGTG | |
| | GTGCACGAGATCGCCAAAATGATGGTGACAAACAACGCCATCGTGGTGCTGGAA | |
| | GATCTGAACTTTGGATTCAAGAGAGGCAGATTCAAAGTGGAAAAGCAGGTGTACC | |
| | AGAAATTCGAGAAGATGCTGATCGACAAACTGAACTTCCTGATGTTCAAAGAGTG | |
| | CAACCAGGCCGCCCTGGGCGGCGTGCGGCGGGCCTATCAGCTGACCGACAAGT | |
| | TCGTGAGCTTCGAGAAGCTGGGAAAGCAGACCGGCTTCCTGTTCTATGTGCCCG | |
| | CCGGCTATACAAGCAAAATCGATCCTACAACCGGTTTCACCAACCTGTTCAATAC | |
| | CAAGAAATGCACCAACGCCGAGGGAAGAAAGGTGTTCTTCGAGGCTATGAACAG | |
| | CATCATCTACGACGGCTCCAGAAAATCTTTCGCCTTTAGCTTCGACTACGGCAAC | |
| | CCCGTGTTTCGAGCCTCCCAGACCAGCTTCAAGAAGGAATGGACCGTGTACAGC | |
| | GCCGATACAAGAATCGTGTATAATCGGGGCGAAAAGACCGTAAACACCATCCAC | |
| | CCTACCCAGATCCTGCACGACGCCCTGTGCGCCTTGGGAATCGACGTGCACGAT | |
| | GGGTTAAATGTCTTGAACGTCGTGAGAGAGACACCCGCTGATAAGATCCACGCC | |
| | AAGTTCTTCAGCGATCTCTTCTACGCCTTCGACAGAACCCTGCAGATGAGGAACT | |
| | CTGTGAGCGGGACCGACGAAGATTACATCCAGAGCCCTGTGCTGAATGCTACCG | |
| | GCGAGTTCTTTGACAGCAGAAAAGCCGACAGCACCCTGCCCCAGGACGCAGAC | |
| | GCTAATGGAGCCTACCACATCGCCCTGAAGGGCCTGCTGCTCCTGCAGAGAATG | |
| | AAGGATATCGGCTCAGATATCAAGCTGGATCTGTCTATTAAGCACGAGGATTGGT | |
| | TCGCCTTCGCTCAGAAGCGGTGCCAGAGAtctagaAAGCGGACAGCAGACGGCTC | |
| | CGAATTTGAAAGCCCTAAGAAAAAGAGAAAGGTGggatccGGCAAACCTATCCCCA | |
| | ATCCCCTGCTGGGCCTGGACAGCACCTGA | |

In some embodiments a ZTAE Type V Cas protein comprises an amino acid sequence of SEQ ID NO: 79, SEQ ID NO:80, or SEQ ID NO:81. In some embodiments, a ZTAE Type V Cas protein has nickase activity, for example resulting from one or more amino acid substitutions relative to the sequence of SEQ ID NO:79, SEQ ID NO: 80, or SEQ ID NO:81. In some embodiments, the one or more amino acid substitutions providing nickase activity comprise a D905 substitution, wherein the position of the D905 substitution is defined with respect to the amino acid numbering of SEQ ID NO:80 (corresponding to amino acid 908 of SEQ ID NO: 121). In some embodiments, the one or more amino acid substitutions providing nickase activity comprise an E990 substitution, wherein the position of the E990 substitution is defined with respect to the amino acid numbering of SEQ ID NO: 80 (corresponding to amino acid 993 of SEQ ID NO: 121). In some embodiments, the one or more amino acid substitutions providing nickase activity comprise a R1206 substitution, wherein the position of the R1206 substitution is defined with respect to the amino acid numbering of SEQ ID NO:80 (corresponding to amino acid 1226 of SEQ ID NO: 121). In some embodiments, the one or more amino acid substitutions providing nickase activity comprise a D1243 substitution, wherein the position of the D1243 substitution is defined with respect to the amino acid numbering of SEQ ID NO:80 (corresponding to amino acid 1263 of SEQ ID NO: 121). In some embodiments, a ZTAE Type V Cas protein is catalytically inactive, for example due to a R1206 substitution in combination with a D905 substitution, a E990 substitution, and/or D1243 substitution.

### 6.2.15. ZSQQ Type V Cas Protein

In one aspect, the disclosure provides ZSQQ Type V Cas proteins. ZSQQ Type V Cas proteins can be further classified as Type V-A Cas proteins. The ZSQQ Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:85. In some embodiments, the ZSQQ Type V Cas proteins comprise an amino acid sequence that is at least 95%, at least 96%, at least 97%, at least 98%, or at least 99% identical to SEQ ID NO:85. In some embodiments, a ZSQQ Type V Cas protein comprises an amino acid sequence that is identical to SEQ ID NO:85.

Exemplary ZSQQ Type V Cas protein sequences and nucleotide sequences encoding exemplary ZSQQ Type V Cas proteins are set forth in Table 10.

TABLE 10
ZSQQ Type V Cas Sequences

| | | SEQ ID |
|---|---|---|
| Name | Sequence | NO. |
| Wildtype | STINKFCGQGNGYSRSITLRNKLIPIGKTEENLKWFLEKDLERAIAYPEIKNLIDNIHRS | 85 |
| amino acid | VIEDTLSKVALNWNEIFNTLAAYQNEKDKKKKAAIKKDLEKLQGCARKKIVDTFKKNP | |
| sequence | DYEKLFKEGLFKELLPELIKTAPVSEIEDKTKALECFNRFSTYFTGFHENRKNMYSED | |
| (without N- | AKSTAISYRIVNENFPKFFANIKLYNYLKEKFPQIIINTEESLKDYLKGKKLDSVFSIDGF | |
| terminal | NDVLAQSGIDFYNTVIGGISGEAGTEKTQGLNEKINLARQQLPKDEKDKLRGKMVDL | |
| methionine) | FKQILSDRETSSFIPTGFENKKEVYSTVKKFSEIVVEKSVSKVKEIFTQNEEYNLNEIF | |
| | VPAKSLTNFSQNIFGNWSILSEGLFLLEKDNVKKQLSEKQIETLHKEIAKKDCSFTEL | |
| | QNAYERWCAENSVDATKNINRYFSIVDLRTKNDSFEKEEINILDEITNAFSKIDFDDIH | |
| | DLQQEKEAATPIKNYLDEVQNLYHHLKLVDYRGEERKDANFYSKLDYILRKDRKDYL | |
| | NLAEVVPLYNKVRNFVTKKPGEVKKIKMMFDCSSLLGGWGTDYETKEAHIFIDSGKY | |
| | YLGIINEKLSKDDVELLKKSSERMITKVIYDFQKPDNKNTPRLFIRSKGTNYAPAVFQY | |
| | NLPIESVIDIYDRGLFKTEYRKINSKVYKESLIKMIDYFKMGFERHESYKHYKFCWKE | |
| | SSKYNDIGEFYKDVINSCYQLNFEKVNYENLLKLVENNKLFLFQIYNKDFAEKKSGKK | |
| | NLHTLYWENLFSEENLKDVCLKLNGEAELFWRKASLDKGKVIVHRMGSILVNRTTSE | |
| | GKSIPEDIYQEIYQYKNKMKDKISDEAKSLLDSGTVICKEATHDITKDKRFTEDTYLFH | |
| | CPITMNFKATDKKNKEFNNHVLEVLKENPDVKIIGLDRGERHLIYLSLINQKGEIELQK | |
| | TLNLVEQVRNDKTVKVDYQEKLVHKEGDRDKARKNWQTIGNIKELKEGYLSAVVHEI | |
| | AMLMVENNAIVVMEDLNFGFKRGRFAVERQIYQKFENMLIEKLNYLVFKDKNATEPG | |
| | GVLNAYQLTNKSANVTDVYKQCGWLFYIPAAYTSKIDPKTGFANLFITKGLTNVEKK | |
| | KEFFDKFDSIRYDSKEDCFVFGFDYAKLCDNASFRKKWEVYTRGERLVYNKDKHKN | |
| | EPINPTEELKGIFDAFDINWNTDDNFIDSVQTIQAEKANAKFFDILLRMFNATLQMRN | |
| | SKTNSSASEDDYLISPVKAEDGTFFDTREELKKGKDAKLPIDSDANGAYHIALKGLFL | |
| | LENDFNRDEKGVIQNISNADWFKFVQEKKYKD | |
| Wildtype | MSTINKFCGQGNGYSRSITLRNKLIPIGKTEENLKWFLEKDLERAIAYPEIKNLIDNIHR | 86 |
| amino acid | SVIEDTLSKVALNWNEIFNTLAAYQNEKDKKKKAAIKKDLEKLQGCARKKIVDTFKKN | |
| sequence (with | PDYEKLFKEGLFKELLPELIKTAPVSEIEDKTKALECFNRFSTYFTGFHENRKNMYSE | |
| N-terminal | DAKSTAISYRIVNENFPKFFANIKLYNYLKEKFPQIIINTEESLKDYLKGKKLDSVFSID | |
| methionine) | GFNDVLAQSGIDFYNTVIGGISGEAGTEKTQGLNEKINLARQQLPKDEKDKLRGKMV | |
| | DLFKQILSDRETSSFIPTGFENKKEVYSTVKKFSEIVVEKSVSKVKEIFTQNEEYNLNE | |
| | IFVPAKSLTNFSQNIFGNWSILSEGLFLLEKDNVKKQLSEKQIETLHKEIAKKDCSFTE | |
| | LQNAYERWCAENSVDATKNINRYFSIVDLRTKNDSFEKEEINILDEITNAFSKIDFDDI | |
| | HDLQQEKEAATPIKNYLDEVQNLYHHLKLVDYRGEERKDANFYSKLDYILRKDRKDY | |
| | LNLAEVVPLYNKVRNFVTKKPGEVKKIKMMFDCSSLLGGWGTDYETKEAHIFIDSGK | |
| | YYLGIINEKLSKDDVELLKKSSERMITKVIYDFQKPDNKNTPRLFIRSKGTNYAPAVFQ | |
| | YNLPIESVIDIYDRGLFKTEYRKINSKVYKESLIKMIDYFKMGFERHESYKHYKFCWK | |
| | ESSKYNDIGEFYKDVINSCYQLNFEKVNYENLLKLVENNKLFLFQIYNKDFAEKKSGK | |
| | KNLHTLYWENLFSEENLKDVCLKLNGEAELFWRKASLDKGKVIVHRMGSILVNRTTS | |
| | EGKSIPEDIYQEIYQYKNKMKDKISDEAKSLLDSGTVICKEATHDITKDKRFTEDTYLF | |
| | HCPITMNFKATDKKNKEFNNHVLEVLKENPDVKIIGLDRGERHLIYLSLINQKGEIELQ | |
| | KTLNLVEQVRNDKTVKVDYQEKLVHKEGDRDKARKNWQTIGNIKELKEGYLSAVVH | |
| | EIAMLMVENNAIVVMEDLNFGFKRGRFAVERQIYQKFENMLIEKLNYLVFKDKNATE | |
| | PGGVLNAYQLTNKSANVTDVYKQCGWLFYIPAAYTSKIDPKTGFANLFITKGLTNVE | |
| | KKKEFFDKFDSIRYDSKEDCFVFGFDYAKLCDNASFRKKWEVYTRGERLVYNKDKH | |
| | KNEPINPTEELKGIFDAFDINWNTDDNFIDSVQTIQAEKANAKFFDILLRMFNATLQM | |
| | RNSKTNSSASEDDYLISPVKAEDGTFFDTREELKKGKDAKLPIDSDANGAYHIALKG | |
| | LFLLENDFNRDEKGVIQNISNADWFKFVQEKKYKD | |
| Expression | MGSGSTINKFCGQGNGYSRSITLRNKLIPIGKTEENLKWFLEKDLERAIAYPEIKNLID | 87 |
| construct (with | NIHRSVIEDTLSKVALNWNEIFNTLAAYQNEKDKKKKAAIKKDLEKLQGCARKKIVDT | |
| N-terminal | FKKNPDYEKLFKEGLFKELLPELIKTAPVSEIEDKTKALECFNRFSTYFTGFHENRKN | |
| methionine, | MYSEDAKSTAISYRIVNENFPKFFANIKLYNYLKEKFPQIIINTEESLKDYLKGKKLDSV | |
| V5-tag and C- | FSIDGFNDVLAQSGIDFYNTVIGGISGEAGTEKTQGLNEKINLARQQLPKDEKDKLRG | |
| terminal NLS) | KMVDLFKQILSDRETSSFIPTGFENKKEVYSTVKKFSEIVVEKSVSKVKEIFTQNEEY | |
| aa sequence | NLNEIFVPAKSLTNFSQNIFGNWSILSEGLFLLEKDNVKKQLSEKQIETLHKEIAKKDC | |
| | SFTELQNAYERWCAENSVDATKNINRYFSIVDLRTKNDSFEKEEINILDEITNAFSKID | |
| | FDDIHDLQQEKEAATPIKNYLDEVQNLYHHLKLVDYRGEERKDANFYSKLDYILRKD | |
| | RKDYLNLAEVVPLYNKVRNFVTKKPGEVKKIKMMFDCSSLLGGWGTDYETKEAHIFI | |
| | DSGKYYLGIINEKLSKDDVELLKKSSERMITKVIYDFQKPDNKNTPRLFIRSKGTNYA | |
| | PAVFQYNLPIESVIDIYDRGLFKTEYRKINSKVYKESLIKMIDYFKMGFERHESYKHYK | |
| | FCWKESSKYNDIGEFYKDVINSCYQLNFEKVNYENLLKLVENNKLFLFQIYNKDFAE | |
| | KKSGKKNLHTLYWENLFSEENLKDVCLKLNGEAELFWRKASLDKGKVIVHRMGSIL | |
| | VNRTTSEGKSIPEDIYQEIYQYKNKMKDKISDEAKSLLDSGTVICKEATHDITKDKRFT | |
| | EDTYLFHCPITMNFKATDKKNKEFNNHVLEVLKENPDVKIIGLDRGERHLIYLSLINQK | |
| | GEIELQKTLNLVEQVRNDKTVKVDYQEKLVHKEGDRDKARKNWQTIGNIKELKEGY | |
| | LSAVVHEIAMLMVENNAIVVMEDLNFGFKRGRFAVERQIYQKFENMLIEKLNYLVFK | |
| | DKNATEPGGVLNAYQLTNKSANVTDVYKQCGWLFYIPAAYTSKIDPKTGFANLFITK | |
| | GLTNVEKKKEFFDKFDSIRYDSKEDCFVFGFDYAKLCDNASFRKKWEVYTRGERLV | |
| | YNKDKHKNEPINPTEELKGIFDAFDINWNTDDNFIDSVQTIQAEKANAKFFDILLRMF | |
| | NATLQMRNSKTNSSASEDDYLISPVKAEDGTFFDTREELKKGKDAKLPIDSDANGAY | |
| | HIALKGLFLLENDFNRDEKGVIQNISNADWFKFVQEKKYKDSRKRTADGSEFESPKK | |
| | KRKVGSGKPIPNPLLGLDST | |
| Wildtype | ATGTCAACTATTAACAAATTTTGTGGACAGGGGAATGGGTATTCTCGTTCAATTA | 88 |
| coding | CTTTGAGGAATAAGTTAATTCCTATTGGAAAAACTGAAGAAAATTTGAAATGGTTT | |
| sequence (with | TTAGAAAAAGATTTGGAAAGGGCAATTGCTTATCCGGAGATAAAGAATCTTATAG | |
| N-terminal | ATAATATTCATCGTAGTGTAATTGAGGATACTTTATCCAAAGTTGCTTTGAATTGG | |
| methionine | AATGAAATATTCAATACACTTGCTGCTTATCAAAATGAAAAAGATAAAAAAAAGAA | |
| and stop | AGCAGCAATAAAAAAGGATTTGGAGAAATTACAAGGTTGTGCAAGAAAGAAAATA | |
| codon) | GTTGATACTTTTAAAAAGAATCCTGATTATGAAAAATTGTTTAAGGAAGGATTATT | |
| | CAAAGAACTATTACCTGAGTTAATAAAAACTGCTCCTGTTAGTGAAATAGAAGAT | |
| | AAAACAAAAGCTTTGGAATGTTTTAATAGATTTAGTACATATTTTACAGGATTTCA | |
| | TGAAAATAGAAAAAATATGTATAGCGAAGATGCAAAATCAACTGCAATAAGTTAC | |
| | CGTATTGTAAATGAGAATTTCCCCAAATTTTTTGCAAATATAAAGTTATATAATTAT | |
| | TTAAAAGAAAAGTTTCCACAAATTATTATTAATACAGAAGAATCTTTAAAAGATTAT | |
| | CTAAAAGGTAAAAAACTTGATTCTGTATTTAGTATTGATGGATTTAATGATGTTTT | |
| | AGCTCAAAGTGGAATCGATTTTTATAATACAGTAATTGGTGGAATTTCTGGTGAA | |
| | GCCGGAACAGAAAAGACTCAAGGATTAAATGAAAAAATCAATCTTGCAAGACAA | |
| | CAATTACCAAAAGATGAAAAAGATAAACTTCGTGGAAAAATGGTTGATTTATTTAA | |
| | GCAGATTTTAAGTGATAGAGAAACATCTTCGTTTATTCCAACTGGTTTTGAAAATA | |
| | AAAAAGAAGTTTATTCTACTGTAAAGAAATTTAGTGAAATTGTTGTTGAAAAGTCT | |
| | GTTTCAAAAGTAAAAGAAATTTTTACACAAAATGAAGAATATAATCTTAATGAAAT | |
| | CTTTGTTCCAGCAAAATCATTAACAAATTTTTCTCAAAATATTTTTGGAAATTGGT | |
| | CTATTTTATCAGAAGGGTTATTTTTGCTTGAAAAAGATAATGTTAAAAAACAATTA | |
| | TCTGAAAAACAAATTGAAACATTACACAAAGAAATTGCAAAAAAAGATTGTTCTTT | |
| | TACTGAACTACAAAATGCTTATGAAAGATGGTGTGCTGAAAATAGTGTTGATGCA | |
| | ACAAAAAATATCAATAGGTATTTTTCAATAGTTGATTTAAGAACAAAAAATGATTC | |
| | GTTTGAAAAAGAAGAAATTAATATTTTGGATGAAATTACAAATGCTTTTTCAAAAA | |
| | TTGATTTTGATGATATTCATGATTTACAACAAGAAAAAGAAGCTGCAACACCAATA | |
| | AAAAATTATTTGGATGAAGTTCAAAATCTTTATCATCACTTAAAACTTGTTGATTAT | |
| | CGTGGTGAAGAACGAAAGGATGCAAACTTTTATTCAAAGCTAGATTATATATTAA | |
| | GGAAAGATAGGAAAGATTACCTTAATCTTGCTGAAGTTGTACCTTTGTATAACAA | |
| | AGTTCGTAATTTTGTAACAAAGAAACCTGGTGAAGTAAAAAAGATTAAAATGATG | |
| | TTTGATTGTAGTTCTTTATTAGGGGGGTGGGGAACTGATTACGAAACAAAAGAA | |
| | GCTCATATTTTTATTGATTCTGGAAAATATTATTTGGGAATTATAAACGAAAAATT | |
| | ATCAAAAGATGATGTTGAGTTATTAAAAAAATCAAGTGAAAGAATGATAACAAAA | |
| | GTAATTTATGATTTTCAGAAACCTGATAATAAAAATACACCTCGTTTATTTATTCG | |
| | TTCAAAAGGAACAAATTATGCACCTGCTGTTTTTCAATATAATTTACCAATAGAAT | |
| | CTGTTATTGATATTTATGATAGAGGATTGTTTAAAACCGAATATAGAAAAATCAAT | |
| | TCAAAAGTTTACAAAGAATCATTAATAAAAATGATTGATTATTTCAAGATGGGCTT | |
| | TGAAAGACATGAATCATATAAGCATTATAAATTCTGTTGGAAGGAATCTTCAAAAT | |
| | ATAATGATATTGGTGAATTTTACAAGGATGTGATAAATTCATGCTATCAATTAAAT | |
| | TTCGAAAAAGTGAATTATGAAAATTTATTAAAATTGGTTGAAAACAATAAATTATT | |
| | CCTTTTCCAAATATATAACAAAGATTTTGCAGAAAAAAAATCTGGAAAGAAAAATC | |
| | TTCATACTTTGTATTGGGAAAATCTTTTTAGTGAAGAAAACTTGAAAGATGTTTGC | |
| | TTAAAATTGAATGGTGAAGCTGAACTTTTCTGGCGCAAAGCAAGTTTAGACAAAG | |
| | GAAAAGTTATAGTTCATAGAATGGGTTCTATTCTTGTAAATAGAACTACATCTGAA | |
| | GGTAAATCAATTCCAGAAGATATTTATCAGGAAATTTATCAATATAAAAATAAAAT | |
| | GAAAGATAAAATTTCTGATGAAGCAAAAAGTCTTTTAGATTCAGGAACAGTTATTT | |
| | GTAAAGAAGCAACTCACGATATTACAAAAGACAAGCGCTTTACAGAAGATACATA | |
| | TCTTTTCCATTGTCCAATTACAATGAACTTTAAAGCAACTGATAAAAAAAATAAAG | |
| | AATTTAATAATCATGTTCTTGAAGTTTTAAAAGAAAATCCAGATGTTAAAATTATTG | |
| | GTCTTGACCGTGGTGAAAGACATTTGATTTATCTTTCTTTGATTAATCAAAAAGGT | |
| | GAAATTGAACTTCAAAAAACATTGAATCTTGTAGAACAAGTTAGAAATGATAAAAC | |
| | TGTAAAAGTAGATTATCAAGAAAAACTTGTACATAAAGAAGGCGACAGAGACAAA | |
| | GCTCGTAAAAACTGGCAAACAATTGGAAATATCAAAGAACTAAAAGAAGGTTATT | |
| | TATCTGCTGTTGTTCATGAAATTGCAATGTTGATGGTAGAAAATAATGCAATTGTT | |
| | GTAATGGAAGATTTGAATTTTGGATTTAAACGTGGTCGATTTGCTGTAGAAAGAC | |
| | AAATTTATCAAAAGTTTGAAAATATGCTCATTGAAAAACTTAATTATCTTGTGTTTA | |
| | AGGATAAAAATGCTACAGAACCAGGTGGTGTCCTTAATGCATATCAATTAACAAA | |
| | TAAATCTGCAAATGTAACTGACGTTTATAAACAATGTGGATGGCTTTTCTATATTC | |
| | CAGCAGCGTATACTTCAAAAATTGATCCAAAAACAGGTTTTGCAAATTTATTCATA | |
| | ACAAAAGGATTAACAAATGTAGAAAAGAAAAAAGAATTCTTTGATAAATTCGATTC | |
| | CATTCGTTATGACTCAAAAGAAGACTGTTTTGTATTTGGTTTTGATTATGCAAAAC | |
| | TTTGTGATAATGCAAGTTTTAGAAAAAAATGGGAAGTATACACAAGAGGGGAAAG | |
| | ATTAGTTTACAATAAAGATAAACATAAAAATGAACCTATTAATCCAACAGAAGAAT | |
| | TAAAAGGAATTTTTGATGCATTCGATATAAATTGGAATACGGATGATAATTTTATT | |
| | GATTCCGTACAGACAATACAAGCAGAAAAAGCAAATGCCAAATTCTTTGATATTC | |
| | TTTTGCGAATGTTTAATGCAACTCTTCAAATGCGAAATTCAAAAACAAATTCTTCA | |
| | GCATCAGAAGATGATTATTTGATATCTCCGGTAAAAGCAGAGGATGGAACATTCT | |
| | TTGATACTCGTGAAGAATTAAAGAAAGGCAAAGATGCAAAACTTCCTATAGATTC | |
| | AGATGCAAACGGAGCTTATCATATTGCACTAAAAGGACTTTTCTTACTTGAAAAT | |
| | GACTTCAATAGAGATGAAAAAGGTGTGATTCAGAATATCTCCAACGCCGATTGG | |
| | TTTAAGTTTGTGCAGGAGAAAAAATACAAAGATTAA | |
| Codon | AGCACCATCAACAAATTCTGCGGCCAGGGCAACGGCTACAGCAGAAGCATCAC | 89 |
| optimized | CCTGCGGAACAAACTGATCCCTATCGGCAAGACTGAGGAGAACCTGAAGTGGT | |
| coding | TCCTGGAGAAGGACCTGGAGCGGGCTATCGCCTACCCCGAGATTAAAAACCTT | |
| sequence (no | ATCGACAATATCCACAGAAGCGTGATAGAGGATACCCTGAGCAAGGTCGCCCT | |
| N-terminal | GAACTGGAATGAGATCTTCAACACCCTGGCCGCCTACCAGAACGAGAAAGATAA | |
| methionine, no | GAAAAAGAAGGCCGCTATCAAGAAGGACCTGGAGAAGTTGCAAGGATGTGCGA | |
| stop codon) | GAAAGAAAATCGTGGATACCTTCAAGAAGAACCCTGATTATGAGAAACTGTTTAA | |
| | AGAGGGACTGTTCAAGGAGCTGCTGCCTGAACTGATCAAGACCGCCCCTGTGA | |
| | GCGAAATTGAAGATAAAACCAAAGCCCTGGAGTGCTTCAACCGGTTCTCCACAT | |
| | ACTTCACCGGCTTCCACGAAAATCGCAAAAATATGTACAGCGAGGACGCGAAGA | |
| | GCACCGCCATCTCCTACCGGATCGTGAACGAGAACTTCCCCAAGTTCTTCGCTA | |
| | ATATCAAGCTGTACAACTACCTCAAGGAAAAATTTCCACAGATTATCATCAACAC | |
| | AGAAGAGTCTCTGAAGGATTACCTGAAGGGCAAGAAGCTGGATTCCGTGTTCTC | |
| | CATCGACGGGTTCAATGACGTGCTGGCCCAGAGCGGCATAGACTTCTACAACA | |
| | CCGTGATCGGTGGCATCTCAGGAGAGGCCGGCACAGAAAAGACCCAGGGCCT | |
| | GAATGAGAAGATCAACCTAGCCAGACAGCAGCTGCCTAAGGATGAGAAGGACA | |
| | AGCTAAGAGGCAAGATGGTCGACCTGTTCAAGCAGATTCTGAGCGATAGAGAAA | |
| | CCAGCAGCTTCATCCCTACTGGCTTCGAGAATAAGAAGGAAGTGTACTCTACCG | |
| | TGAAGAAGTTCAGCGAAATCGTGGTCGAAAAAAGCGTGTCCAAGGTGAAGGAG | |
| | ATCTTCACTCAGAACGAAGAGTACAATCTGAACGAGATCTTCGTGCCTGCGAAG | |
| | AGCCTGACCAATTTTAGCCAGAACATCTTTGGCAACTGGAGCATCCTTTCTGAA | |
| | GGCCTGTTCCTGCTGGAAAAGGACAACGTGAAGAAACAGCTGAGTGAGAAACA | |
| | AATCGAGACACTCCATAAGGAGATCGCCAAGAAGGACTGCAGCTTTACCGAACT | |
| | GCAGAACGCCTACGAGCGGTGGTGCGCCGAGAACTCCGTGGACGCCACCAAG | |
| | AACATTAACAGATACTTCAGCATCGTCGACCTGAGAACCAAGAATGACTCCTTC | |
| | GAGAAGGAAGAGATCAATATCCTTGATGAGATAACCAACGCCTTCTCTAAGATT | |
| | GACTTCGACGATATCCACGATCTGCAGCAAGAGAAGGAGGCCGCCACCCCTAT | |
| | CAAGAACTACCTGGACGAGGTTCAAAACCTGTACCACCACCTGAAGCTGGTGGA | |
| | CTACAGAGGTGAGGAACGAAAGGACGCTAACTTCTACTCTAAACTGGACTATAT | |
| | CCTGAGAAAGGACAGAAAGGACTACCTGAACCTGGCCGAAGTGGTGCCATTGT | |
| | ACAACAAGGTTAGAAACTTCGTGACCAAGAAGCCTGGCGAGGTGAAAAAGATCA | |
| | AGATGATGTTCGACTGCAGCAGCCTGCTGGGCGGATGGGGCACAGATTACGAG | |
| | ACAAAAGAGGCCCACATTTTCATCGACTCCGGCAAGTATTACCTTGGAATCATCA | |
| | ACGAGAAGTTGTCAAAAGATGACGTGGAGCTGCTGAAGAAGAGCAGCGAACGG | |
| | ATGATCACAAAGGTGATCTACGATTTCCAAAAGCCCGATAACAAGAATACACCTA | |
| | GACTGTTCATCAGGAGCAAGGGCACAAATTATGCTCCTGCTGTTTTCCAATACAA | |
| | TCTGCCAATAGAGTCTGTGATCGATATTTACGACCGTGGCCTGTTTAAGACCGA | |
| | GTACAGAAAAATCAACAGCAAGGTGTACAAGGAGAGCCTGATTAAGATGATCGA | |
| | TTACTTCAAGATGGGCTTTGAGAGACACGAGAGCTACAAGCACTACAAGTTTTG | |
| | CTGGAAGGAATCTAGCAAGTACAACGACATCGGCGAATTTTACAAGGATGTGAT | |
| | TAACTCTTGTTACCAGCTGAACTTCGAGAAGGTGAACTATGAGAACCTCCTGAA | |
| | GTTAGTGGAAAACAACAAGCTGTTCCTGTTTCAGATCTACAACAAGGATTTTGCC | |
| | GAAAAGAAAAGCGGTAAGAAGAACCTGCACACCCTGTACTGGGAGAACCTGTTT | |
| | TCTGAGGAGAACCTGAAGGACGTTTGTCTGAAGCTGAATGGCGAGGCCGAGCT | |
| | GTTCTGGCGGAAGGCTTCTCTGGACAAGGGCAAGGTGATCGTGCACAGAATGG | |
| | GCTCTATCCTGGTGAACAGAACAACAAGCGAGGGCAAGTCAATCCCTGAGGAC | |
| | ATCTACCAGGAGATCTATCAGTACAAGAACAAAATGAAGGATAAGATCAGCGAC | |
| | GAAGCCAAAAGCCTGCTGGACAGCGGCACCGTGATCTGTAAAGAAGCCACCCA | |
| | CGACATCACCAAGGACAAACGGTTCACAGAGGACACCTACCTGTTCCACTGCCC | |
| | TATCACCATGAACTTCAAGGCCACCGACAAGAAAAACAAAGAGTTCAACAACCA | |
| | CGTGCTGGAAGTGCTGAAAGAGAATCCCGACGTGAAGATCATCGGCCTGGACA | |
| | GAGGCGAACGGCACCTGATCTACCTGAGCCTGATCAACCAGAAGGGCGAGATC | |
| | GAGCTGCAGAAAACCCTGAATCTGGTGGAACAGGTGCGGAACGACAAAACCGT | |
| | GAAGGTGGACTACCAGGAGAAGCTGGTGCATAAGGAAGGCGACCGCGACAAA | |
| | GCCAGAAAGAACTGGCAGACAATCGGAAACATCAAGGAACTGAAGGAGGGCTA | |
| | CCTGTCTGCCGTGGTGCACGAAATCGCCATGCTGATGGTGGAAAACAACGCCA | |
| | TCGTGGTGATGGAGGACCTGAACTTCGGCTTCAAGAGAGGCAGATTCGCCGTG | |
| | GAACGGCAGATCTACCAGAAGTTCGAGAACATGCTGATCGAAAAGCTGAACTAC | |
| | CTAGTGTTCAAGGACAAGAACGCCACCGAACCTGGCGGCGTGCTGAATGCGTA | |
| | TCAGCTCACCAACAAGAGCGCCAACGTCACCGACGTGTACAAACAGTGCGGCT | |
| | GGCTGTTCTACATCCCCGCCGCTTATACAAGCAAGATCGACCCCAAGACCGGAT | |
| | TCGCCAACCTGTTCATCACAAAGGGACTGACAAACGTGGAAAAGAAGAAGGAGT | |
| | TCTTCGATAAGTTCGACAGCATCCGGTACGACAGCAAAGAGGACTGCTTTGTGT | |
| | TCGGCTTCGACTACGCCAAGCTGTGCGACAACGCCTCCTTTAGAAAGAAGTGG | |
| | GAAGTTTACACCAGAGGAGAGAGGCTGGTCTACAACAAAGACAAGCACAAAAAC | |
| | GAACCTATCAACCCCACCGAGGAGCTGAAGGGCATCTTCGATGCTTTTGATATT | |
| | AACTGGAACACCGACGACAACTTCATTGATTCAGTGCAGACCATCCAGGCCGAG | |
| | AAGGCCAACGCCAAGTTCTTTGACATCCTGCTGAGAATGTTCAACGCCACACTG | |
| | CAGATGAGAAACAGCAAGACTAACTCCTCTGCCAGCGAGGACGACTACCTGATC | |
| | AGCCCTGTCAAAGCCGAGGATGGCACCTTCTTCGACACAAGAGAGGAATTAAAG | |
| | AAGGGCAAAGATGCCAAGCTGCCGATCGACAGCGACGCTAATGGCGCCTACCA | |
| | CATCGCCCTGAAAGGACTGTTCCTGCTGGAAAATGACTTTAACCGGGACGAGAA | |
| | GGGAGTGATCCAAAATATCAGCAACGCTGATTGGTTCAAGTTTGTGCAGGAGAA | |
| | GAAATACAAGGAT | |
| Expression | ATGggctccggaAGCACCATCAACAAATTCTGCGGCCAGGGCAACGGCTACAGCAG | 90 |
| construct (with | AAGCATCACCCTGCGGAACAAACTGATCCCTATCGGCAAGACTGAGGAGAACCT | |
| N-terminal | GAAGTGGTTCCTGGAGAAGGACCTGGAGCGGGCTATCGCCTACCCCGAGATTA | |
| methionine | AAAACCTTATCGACAATATCCACAGAAGCGTGATAGAGGATACCCTGAGCAAGG | |
| and stop | TCGCCCTGAACTGGAATGAGATCTTCAACACCCTGGCCGCCTACCAGAACGAG | |
| codon, | AAAGATAAGAAAAAGAAGGCCGCTATCAAGAAGGACCTGGAGAAGTTGCAAGG | |
| includes V5- | ATGTGCGAGAAAGAAAATCGTGGATACCTTCAAGAAGAACCCTGATTATGAGAA | |
| tag and C- | ACTGTTTAAAGAGGGACTGTTCAAGGAGCTGCTGCCTGAACTGATCAAGACCGC | |
| terminal NLS) | CCCTGTGAGCGAAATTGAAGATAAAACCAAAGCCCTGGAGTGCTTCAACCGGTT | |
| | CTCCACATACTTCACCGGCTTCCACGAAAATCGCAAAAATATGTACAGCGAGGA | |
| | CGCGAAGAGCACCGCCATCTCCTACCGGATCGTGAACGAGAACTTCCCCAAGT | |
| | TCTTCGCTAATATCAAGCTGTACAACTACCTCAAGGAAAAATTTCCACAGATTAT | |
| | CATCAACACAGAAGAGTCTCTGAAGGATTACCTGAAGGGCAAGAAGCTGGATTC | |
| | CGTGTTCTCCATCGACGGGTTCAATGACGTGCTGGCCCAGAGCGGCATAGACT | |
| | TCTACAACACCGTGATCGGTGGCATCTCAGGAGAGGCCGGCACAGAAAAGACC | |
| | CAGGGCCTGAATGAGAAGATCAACCTAGCCAGACAGCAGCTGCCTAAGGATGA | |
| | GAAGGACAAGCTAAGAGGCAAGATGGTCGACCTGTTCAAGCAGATTCTGAGCG | |
| | ATAGAGAAACCAGCAGCTTCATCCCTACTGGCTTCGAGAATAAGAAGGAAGTGT | |
| | ACTCTACCGTGAAGAAGTTCAGCGAAATCGTGGTCGAAAAAAGCGTGTCCAAGG | |
| | TGAAGGAGATCTTCACTCAGAACGAAGAGTACAATCTGAACGAGATCTTCGTGC | |
| | CTGCGAAGAGCCTGACCAATTTTAGCCAGAACATCTTTGGCAACTGGAGCATCC | |
| | TTTCTGAAGGCCTGTTCCTGCTGGAAAAGGACAACGTGAAGAAACAGCTGAGTG | |
| | AGAAACAAATCGAGACACTCCATAAGGAGATCGCCAAGAAGGACTGCAGCTTTA | |
| | CCGAACTGCAGAACGCCTACGAGCGGTGGTGCGCCGAGAACTCCGTGGACGC | |
| | CACCAAGAACATTAACAGATACTTCAGCATCGTCGACCTGAGAACCAAGAATGA | |
| | CTCCTTCGAGAAGGAAGAGATCAATATCCTTGATGAGATAACCAACGCCTTCTCT | |
| | AAGATTGACTTCGACGATATCCACGATCTGCAGCAAGAGAAGGAGGCCGCCAC | |
| | CCCTATCAAGAACTACCTGGACGAGGTTCAAAACCTGTACCACCACCTGAAGCT | |
| | GGTGGACTACAGAGGTGAGGAACGAAAGGACGCTAACTTCTACTCTAAACTGGA | |
| | CTATATCCTGAGAAAGGACAGAAAGGACTACCTGAACCTGGCCGAAGTGGTGC | |
| | CATTGTACAACAAGGTTAGAAACTTCGTGACCAAGAAGCCTGGCGAGGTGAAAA | |
| | AGATCAAGATGATGTTCGACTGCAGCAGCCTGCTGGGCGGATGGGGCACAGAT | |
| | TACGAGACAAAAGAGGCCCACATTTTCATCGACTCCGGCAAGTATTACCTTGGA | |
| | ATCATCAACGAGAAGTTGTCAAAAGATGACGTGGAGCTGCTGAAGAAGAGCAGC | |
| | GAACGGATGATCACAAAGGTGATCTACGATTTCCAAAAGCCCGATAACAAGAAT | |
| | ACACCTAGACTGTTCATCAGGAGCAAGGGCACAAATTATGCTCCTGCTGTTTTC | |
| | CAATACAATCTGCCAATAGAGTCTGTGATCGATATTTACGACCGTGGCCTGTTTA | |
| | AGACCGAGTACAGAAAAATCAACAGCAAGGTGTACAAGGAGAGCCTGATTAAGA | |
| | TGATCGATTACTTCAAGATGGGCTTTGAGAGACACGAGAGCTACAAGCACTACA | |
| | AGTTTTGCTGGAAGGAATCTAGCAAGTACAACGACATCGGCGAATTTTACAAGG | |
| | ATGTGATTAACTCTTGTTACCAGCTGAACTTCGAGAAGGTGAACTATGAGAACCT | |
| | CCTGAAGTTAGTGGAAAACAACAAGCTGTTCCTGTTTCAGATCTACAACAAGGAT | |
| | TTTGCCGAAAAGAAAAGCGGTAAGAAGAACCTGCACACCCTGTACTGGGAGAAC | |
| | CTGTTTTCTGAGGAGAACCTGAAGGACGTTTGTCTGAAGCTGAATGGCGAGGCC | |
| | GAGCTGTTCTGGCGGAAGGCTTCTCTGGACAAGGGCAAGGTGATCGTGCACAG | |
| | AATGGGCTCTATCCTGGTGAACAGAACAACAAGCGAGGGCAAGTCAATCCCTGA | |
| | GGACATCTACCAGGAGATCTATCAGTACAAGAACAAAATGAAGGATAAGATCAG | |
| | CGACGAAGCCAAAAGCCTGCTGGACAGCGGCACCGTGATCTGTAAAGAAGCCA | |
| | CCCACGACATCACCAAGGACAAACGGTTCACAGAGGACACCTACCTGTTCCACT | |
| | GCCCTATCACCATGAACTTCAAGGCCACCGACAAGAAAAACAAAGAGTTCAACA | |
| | ACCACGTGCTGGAAGTGCTGAAAGAGAATCCCGACGTGAAGATCATCGGCCTG | |
| | GACAGAGGCGAACGGCACCTGATCTACCTGAGCCTGATCAACCAGAAGGGCGA | |
| | GATCGAGCTGCAGAAAACCCTGAATCTGGTGGAACAGGTGCGGAACGACAAAA | |
| | CCGTGAAGGTGGACTACCAGGAGAAGCTGGTGCATAAGGAAGGCGACCGCGA | |
| | CAAAGCCAGAAAGAACTGGCAGACAATCGGAAACATCAAGGAACTGAAGGAGG | |
| | GCTACCTGTCTGCCGTGGTGCACGAAATCGCCATGCTGATGGTGGAAAACAAC | |
| | GCCATCGTGGTGATGGAGGACCTGAACTTCGGCTTCAAGAGAGGCAGATTCGC | |
| | CGTGGAACGGCAGATCTACCAGAAGTTCGAGAACATGCTGATCGAAAAGCTGAA | |
| | CTACCTAGTGTTCAAGGACAAGAACGCCACCGAACCTGGCGGCGTGCTGAATG | |
| | CGTATCAGCTCACCAACAAGAGCGCCAACGTCACCGACGTGTACAAACAGTGC | |
| | GGCTGGCTGTTCTACATCCCCGCCGCTTATACAAGCAAGATCGACCCCAAGACC | |
| | GGATTCGCCAACCTGTTCATCACAAAGGGACTGACAAACGTGGAAAAGAAGAAG | |
| | GAGTTCTTCGATAAGTTCGACAGCATCCGGTACGACAGCAAAGAGGACTGCTTT | |
| | GTGTTCGGCTTCGACTACGCCAAGCTGTGCGACAACGCCTCCTTTAGAAAGAAG | |
| | TGGGAAGTTTACACCAGAGGAGAGAGGCTGGTCTACAACAAAGACAAGCACAA | |
| | AAACGAACCTATCAACCCCACCGAGGAGCTGAAGGGCATCTTCGATGCTTTTGA | |
| | TATTAACTGGAACACCGACGACAACTTCATTGATTCAGTGCAGACCATCCAGGC | |
| | CGAGAAGGCCAACGCCAAGTTCTTTGACATCCTGCTGAGAATGTTCAACGCCAC | |
| | ACTGCAGATGAGAAACAGCAAGACTAACTCCTCTGCCAGCGAGGACGACTACCT | |
| | GATCAGCCCTGTCAAAGCCGAGGATGGCACCTTCTTCGACACAAGAGAGGAATT | |
| | AAAGAAGGGCAAAGATGCCAAGCTGCCGATCGACAGCGACGCTAATGGCGCCT | |
| | ACCACATCGCCCTGAAAGGACTGTTCCTGCTGGAAAATGACTTTAACCGGGACG | |
| | AGAAGGGAGTGATCCAAAATATCAGCAACGCTGATTGGTTCAAGTTTGTGCAGG | |
| | AGAAGAAATACAAGGATtctagaAAGCGGACAGCAGACGGCTCCGAATTTGAAAGC | |
| | CCTAAGAAAAAGAGAAAGGTGggatccGGCAAACCTATCCCCAATCCCCTGCTGG | |
| | GCCTGGACAGCACCTGA | |

In some embodiments a ZSQQ Type V Cas protein comprises an amino acid sequence of SEQ ID NO: 85, SEQ ID NO:86, or SEQ ID NO:87. In some embodiments, a ZSQQ Type V Cas protein has nickase activity, for example resulting from one or more amino acid substitutions relative to the sequence of SEQ ID NO: 85, SEQ ID NO: 86, or SEQ ID NO:87. In some embodiments, the one or more amino acid substitutions providing nickase activity comprise a D913 substitution, wherein the position of the D913 substitution is defined with respect to the amino acid numbering of SEQ ID NO:86 (corresponding to amino acid 908 of SEQ ID NO: 121). In some embodiments, the one or more amino acid substitutions providing nickase activity comprise an E1006 substitution, wherein the position of the E1006 substitution is defined with respect to the amino acid numbering of SEQ ID NO:86 (corresponding to amino acid 993 of SEQ ID NO: 121). In some embodiments, the one or more amino acid substitutions providing nickase activity comprise a R1219 substitution, wherein the position of the R1219 substitution is defined with respect to the amino acid numbering of SEQ ID NO: 86 (corresponding to amino acid 1226 of SEQ ID NO: 121). In some embodiments, the one or more amino acid substitutions providing nickase activity comprise a D1264 substitution, wherein the position of the D1264 substitution is defined with respect to the amino acid numbering of SEQ ID NO: 86 (corresponding to amino acid 1263 of SEQ ID NO:121). In some embodiments, a ZSQQ Type V Cas protein is catalytically inactive, for example due to a R1219 substitution in combination with a D913 substitution, a E1006 substitution, and/or D1264 substitution.

### 6.2.16. ZSYN Type V Cas Protein

In one aspect, the disclosure provides ZSYN Type V Cas proteins. ZSYN Type V Cas proteins can be further classified as Type V-A Cas proteins. The ZSYN Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:91. In some embodiments, the ZSYN Type V Cas proteins comprise an amino acid sequence that is at least 95%, at least 96%, at least 97%, at least 98%, or at least 99% identical to SEQ ID NO:91. In some embodiments, a ZSYN Type V Cas protein comprises an amino acid sequence that is identical to SEQ ID NO:91.

Exemplary ZSYN Type V Cas protein sequences and nucleotide sequences encoding exemplary ZSYN Type V Cas proteins are set forth in Table 1P.

TABLE 1P
ZSYN Type V Cas Sequences

| | | SEQ |
|---|---|---|
| | | ID |
| Name | Sequence | NO. |
| Wildtype | GKFFETDEFIGQYSINKTLRFELIPQGKTKELLNNYMNDNSKIKQDILRADEKNNFKEVI | 91 |
| amino acid | DEYYRELIHDALTDEDIFSITPLVKDAYELYIASRKNTSDSSKKEYRDVKNKIRKEIANILN | |
| sequence | KYKTIYGLDKFANIYKSESDKSVDDDESDNDDLDEKNTTNDDNAKSEDKRIYKWLNKK | |
| (without N- | LRLKQISNEEYDRYYKSLNEYHGFTTGLQGLQNNKENMFSSENKSTAIAFRIIDDNMEK | |
| terminal | YFSNILLLEFIKNKYKDLYEKIEEKANKMNVECFTKYFTQEGIDEYNQMIGRSIEEEYAK | |
| methionine) | GINQEINLYKQSKGLNNKEIRTLSPLYKQILSKTSQNEIIVFKNDKETLEYIKNICDYIIEED | |
| | IFGKMNHLIKTNLIDMCTGIYIKRNELSNISFKLYNDWGLLDRIICDYANEFKTKKEKNEF | |
| | EKLNKEVISLNLLNDIFNKYKETRGNDTDLKEIVEYFKNVDEKMIEDEYSKIKSILNLERID | |
| | IDRRVPSKDEEKGGEGFEQICMIKTFLDLLLESIHIYKPLSLIKNGEKVEIYNYNENFYNE | |
| | YDILFSQLDNIINLYNKVRNYFSKKTYSKEKIKIYFSKPTLLNGWDVNKEISNYSIILRKDE | |
| | EYFLAIMNSDNKIFTNERLEENCAITENNEECYEKMVYKQISDSNKMFSKVFFSEKNKKI | |
| | YMPSEEIKNIRKNKTHLKVANNKDSQTKWIKFMIECYYKHPEWSKYFDINFKKPEEYES | |
| | IVEFYNQVNEKIYNIKFVNIKCDYINSMVDSGELYLFKIYNKDFSKNKKKSGTDNLHTMY | |
| | WKLLFSKENMNCGVYKLNGQAEVFFRKASLPDKITHERNKEIDNKNPIKDKKTSTFTY | |
| | DLKKDKRFMEDKFFFHCPITINYKGLNAKDKEIRKYNEKINKFIAGNPDINIIGIDRGERH | |
| | LLYYTIINQKGEILKQSTLNNVGIEGRDKDYQELLSNKEKERHLARKSWGTIGNIKELKE | |
| | GYLSIWVHELAKLVKEYNAIIVLENLNAGFKRGRTKVEKQVYQKFELALIKKLNYLVFKN | |
| | ENIQNKGGYLKGLQLTQPFDTFKDIGNQSGIIYYVIPSYTSKICPTTGFIDVIKPQYESVE | |
| | KAKELFSKFKRIYFDNNKKCFIFEFMYKDFGRDYGLDKIWSICTLGEKRYYYDSKNKVS | |
| | NVINVTESIISILQEKNINYINSDNIIDEILQYSDVKLYKELLFNLKVVLQMRYTKSGTNNE | |
| | DDFILSPVLDENDKAFCSLNAKETEPQNADANGAYHIAMKGLNAIMSIKNGNVDRDINN | |
| | LENWINFIQKFHIGK | |
| Wildtype | MGKFFETDEFIGQYSINKTLRFELIPQGKTKELLNNYMNDNSKIKQDILRADEKNNFKEV | 92 |
| amino acid | IDEYYRELIHDALTDEDIFSITPLVKDAYELYIASRKNTSDSSKKEYRDVKNKIRKEIANIL | |
| sequence (with | NKYKTIYGLDKFANIYKSESDKSVDDDESDNDDLDEKNTTNDDNAKSEDKRIYKWLNK | |
| N-terminal | KLRLKQISNEEYDRYYKSLNEYHGFTTGLQGLQNNKENMFSSENKSTAIAFRIIDDNME | |
| methionine) | KYFSNILLLEFIKNKYKDLYEKIEEKANKMNVECFTKYFTQEGIDEYNQMIGRSIEEEYA | |
| | KGINQEINLYKQSKGLNNKEIRTLSPLYKQILSKTSQNEIIVFKNDKETLEYIKNICDYIIEE | |
| | DIFGKMNHLIKTNLIDMCTGIYIKRNELSNISFKLYNDWGLLDRIICDYANEFKTKKEKNE | |
| | FEKLNKEVISLNLLNDIFNKYKETRGNDTDLKEIVEYFKNVDEKMIEDEYSKIKSILNLERI | |
| | DIDRRVPSKDEEKGGEGFEQICMIKTFLDLLLESIHIYKPLSLIKNGEKVEIYNYNENFYN | |
| | EYDILFSQLDNIINLYNKVRNYFSKKTYSKEKIKIYFSKPTLLNGWDVNKEISNYSIILRKD | |
| | EEYFLAIMNSDNKIFTNERLEENCAITENNEECYEKMVYKQISDSNKMFSKVFFSEKNK | |
| | KIYMPSEEIKNIRKNKTHLKVANNKDSQTKWIKFMIECYYKHPEWSKYFDINFKKPEEY | |
| | ESIVEFYNQVNEKIYNIKFVNIKCDYINSMVDSGELYLFKIYNKDFSKNKKKSGTDNLHT | |
| | MYWKLLFSKENMNCGVYKLNGQAEVFFRKASLPDKITHERNKEIDNKNPIKDKKTSTF | |
| | TYDLKKDKRFMEDKFFFHCPITINYKGLNAKDKEIRKYNEKINKFIAGNPDINIIGIDRGE | |
| | RHLLYYTIINQKGEILKQSTLNNVGIEGRDKDYQELLSNKEKERHLARKSWGTIGNIKEL | |
| | KEGYLSIWVHELAKLVKEYNAIIVLENLNAGFKRGRTKVEKQVYQKFELALIKKLNYLVF | |
| | KNENIQNKGGYLKGLQLTQPFDTFKDIGNQSGIIYYVIPSYTSKICPTTGFIDVIKPQYES | |
| | VEKAKELFSKFKRIYFDNNKKCFIFEFMYKDFGRDYGLDKIWSICTLGEKRYYYDSKNK | |
| | VSNVINVTESIISILQEKNINYINSDNIIDEILQYSDVKLYKELLFNLKVVLQMRYTKSGTN | |
| | NEDDFILSPVLDENDKAFCSLNAKETEPQNADANGAYHIAMKGLNAIMSIKNGNVDRDI | |
| | NNLENWINFIQKFHIGK | |
| Expression | MGSGGKFFETDEFIGQYSINKTLRFELIPQGKTKELLNNYMNDNSKIKQDILRADEKNN | 93 |
| construct (with | FKEVIDEYYRELIHDALTDEDIFSITPLVKDAYELYIASRKNTSDSSKKEYRDVKNKIRKEI | |
| N-terminal | ANILNKYKTIYGLDKFANIYKSESDKSVDDDESDNDDLDEKNTTNDDNAKSEDKRIYKW | |
| methionine, | LNKKLRLKQISNEEYDRYYKSLNEYHGFTTGLQGLQNNKENMFSSENKSTAIAFRIIDD | |
| V5-tag and C- | NMEKYFSNILLLEFIKNKYKDLYEKIEEKANKMNVECFTKYFTQEGIDEYNQMIGRSIEE | |
| terminal NLS) | EYAKGINQEINLYKQSKGLNNKEIRTLSPLYKQILSKTSQNEIIVFKNDKETLEYIKNICDY | |
| aa sequence | IIEEDIFGKMNHLIKTNLIDMCTGIYIKRNELSNISFKLYNDWGLLDRIICDYANEFKTKKE | |
| | KNEFEKLNKEVISLNLLNDIFNKYKETRGNDTDLKEIVEYFKNVDEKMIEDEYSKIKSILN | |
| | LERIDIDRRVPSKDEEKGGEGFEQICMIKTFLDLLLESIHIYKPLSLIKNGEKVEIYNYNEN | |
| | FYNEYDILFSQLDNIINLYNKVRNYFSKKTYSKEKIKIYFSKPTLLNGWDVNKEISNYSIIL | |
| | RKDEEYFLAIMNSDNKIFTNERLEENCAITENNEECYEKMVYKQISDSNKMFSKVFFSE | |
| | KNKKIYMPSEEIKNIRKNKTHLKVANNKDSQTKWIKFMIECYYKHPEWSKYFDINFKKP | |
| | EEYESIVEFYNQVNEKIYNIKFVNIKCDYINSMVDSGELYLFKIYNKDFSKNKKKSGTDN | |
| | LHTMYWKLLFSKENMNCGVYKLNGQAEVFFRKASLPDKITHERNKEIDNKNPIKDKKT | |
| | STFTYDLKKDKRFMEDKFFFHCPITINYKGLNAKDKEIRKYNEKINKFIAGNPDINIIGIDR | |
| | GERHLLYYTIINQKGEILKQSTLNNVGIEGRDKDYQELLSNKEKERHLARKSWGTIGNIK | |
| | ELKEGYLSIWVHELAKLVKEYNAIIVLENLNAGFKRGRTKVEKQVYQKFELALIKKLNYLV | |
| | FKNENIQNKGGYLKGLQLTQPFDTFKDIGNQSGIIYYVIPSYTSKICPTTGFIDVIKPQYE | |
| | SVEKAKELFSKFKRIYFDNNKKCFIFEFMYKDFGRDYGLDKIWSICTLGEKRYYYDSKN | |
| | KVSNVINVTESIISILQEKNINYINSDNIIDEILQYSDVKLYKELLFNLKVVLQMRYTKSGTN | |
| | NEDDFILSPVLDENDKAFCSLNAKETEPQNADANGAYHIAMKGLNAIMSIKNGNVDRDI | |
| | NNLENWINFIQKFHIGKSRKRTADGSEFESPKKKRKVGSGKPIPNPLLGLDST | |
| Wildtype | ATGGGTAAATTTTTTGAAACAGATGAATTTATTGGACAGTATTCAATAAATAAAACAT | 94 |
| coding | TACGATTCGAATTGATACCACAAGGTAAGACAAAGGAATTACTAAATAATTATATGA | |
| sequence (with | ATGATAACAGCAAAATTAAACAGGATATTTTAAGAGCAGATGAAAAGAATAATTTTA | |
| N-terminal | AAGAAGTAATTGATGAATATTATCGAGAGTTGATTCATGATGCTTTAACAGATGAAG | |
| methionine | ATATTTTTTCCATTACACCATTAGTAAAGGATGCATATGAATTATATATTGCTTCTAG | |
| and stop | AAAAAATACTTCTGATAGTTCTAAAAAAGAATATAGAGATGTTAAAAATAAAATTAG | |
| codon) | GAAAGAAATAGCAAACATTCTTAATAAATATAAGACGATTTATGGACTAGATAAATT | |
| | TGCAAATATATATAAATCCGAGAGTGATAAAAGTGTAGATGATGATGAATCTGATAA | |
| | TGATGATTTAGATGAGAAAAATACTACTAATGATGATAATGCAAAATCAGAAGATAA | |
| | AAGGATATACAAATGGCTAAATAAAAAATTAAGATTAAAACAAATTTCTAACGAGGA | |
| | ATATGATAGATACTACAAATCTTTAAATGAATATCATGGTTTTACAACAGGTCTGCA | |
| | AGGATTACAAAATAATAAAGAAAATATGTTCTCTTCAGAAAACAAAAGTACGGCAAT | |
| | AGCATTTCGAATAATAGATGACAATATGGAAAAATATTTTTCAAATATACTGTTATTA | |
| | GAATTTATTAAAAACAAATATAAAGATTTATATGAAAAAATTGAAGAAAAAGCAAATA | |
| | AAATGAATGTGGAATGTTTTACTAAATATTTTACACAAGAGGGTATAGATGAATATA | |
| | ATCAAATGATAGGTAGAAGTATAGAAGAAGAATATGCAAAAGGTATAAATCAAGAA | |
| | ATAAATCTTTATAAACAATCAAAAGGATTAAATAATAAAGAAATTAGGACATTATCTC | |
| | CATTGTATAAGCAAATATTATCAAAGACTTCACAAAATGAAATAATAGTATTCAAAAA | |
| | TGATAAAGAAACTTTAGAATACATCAAGAATATATGTGATTATATAATAGAAGAAGA | |
| | TATATTTGGAAAGATGAATCATTTAATTAAAACAAATTTGATTGATATGTGTACTGGT | |
| | ATATATATAAAAAGAAATGAATTATCGAATATTTCATTTAAACTTTATAATGATTGGG | |
| | GATTACTAGATAGAATAATATGTGATTATGCAAATGAATTTAAGACAAAAAAAGAAA | |
| | AGAACGAATTTGAAAAATTAAATAAAGAAGTAATTTCACTTAATCTTTTAAATGATAT | |
| | ATTTAATAAATATAAGGAAACAAGAGGGAATGATACAGATTTAAAAGAAATAGTAGA | |
| | ATATTTTAAAAATGTAGATGAAAAAATGATAGAGGATGAATACTCTAAAATAAAAAG | |
| | TATTTTAAATTTAGAAAGAATAGATATTGATAGAAGAGTACCAAGCAAAGATGAAGA | |
| | AAAAGGTGGAGAAGGATTTGAACAAATTTGTATGATAAAAACATTTTTAGATTTATT | |
| | GCTTGAGAGTATACATATTTACAAACCATTAAGTTTAATTAAAAATGGAGAGAAAGT | |
| | GGAGATATATAATTATAATGAAAATTTTTACAATGAATATGATATATTGTTTTCACAA | |
| | TTAGATAATATAATTAACTTATATAATAAAGTCAGAAATTATTTTTCTAAAAAAACATA | |
| | TTCAAAAGAAAAAATCAAGATATATTTTTCTAAGCCAACGTTATTAAATGGATGGGA | |
| | TGTAAATAAAGAAATATCAAATTATTCGATTATTTTGAGAAAAGATGAAGAATATTTC | |
| | CTAGCCATAATGAATAGTGATAATAAGATTTTTACTAATGAAAGATTGGAAGAAAAT | |
| | TGCGCAATTACAGAAAATAATGAAGAGTGTTATGAAAAAATGGTATATAAACAAATA | |
| | TCCGATTCAAATAAGATGTTTTCAAAAGTGTTTTTTTCAGAAAAAAACAAAAAAATAT | |
| | ATATGCCTTCAGAAGAAATTAAAAATATTAGAAAAAATAAAACACATTTGAAAGTAG | |
| | CAAATAATAAAGACTCACAAACAAAATGGATTAAATTTATGATTGAATGCTATTATAA | |
| | ACATCCTGAATGGAGTAAATATTTTGATATAAATTTTAAAAAGCCTGAAGAATATGA | |
| | ATCAATAGTTGAATTTTATAATCAAGTAAATGAAAAAATATATAATATAAAATTTGTA | |
| | AATATTAAATGTGATTATATAAATAGTATGGTTGATAGTGGAGAATTGTATTTGTTTA | |
| | AAATATATAATAAGGATTTTTCAAAAAATAAGAAAAAATCTGGAACAGATAATTTACA | |
| | CACTATGTATTGGAAATTATTATTTTCAAAAGAAAATATGAATTGTGGTGTATACAAA | |
| | TTAAATGGACAAGCAGAAGTGTTTTTTAGGAAAGCTTCTTTACCTGATAAAATTACA | |
| | CATGAAAGAAATAAAGAAATAGATAATAAAAATCCAATAAAAGATAAAAAAACAAGT | |
| | ACATTTACTTATGATTTAAAGAAAGATAAAAGATTCATGGAAGATAAATTCTTCTTTC | |
| | ATTGCCCAATAACAATAAATTATAAAGGATTAAATGCAAAAGATAAAGAAATAAGAA | |
| | AATATAATGAGAAAATAAACAAATTTATTGCTGGTAACCCAGATATAAATATTATCG | |
| | GAATAGATCGTGGTGAACGACATTTGCTATATTATACGATAATAAATCAAAAGGGT | |
| | GAAATATTAAAACAGTCAACATTAAATAATGTTGGTATTGAAGGGCGTGATAAAGAT | |
| | TATCAAGAATTATTATCTAATAAAGAGAAAGAACGTCACTTAGCTAGAAAAAGTTGG | |
| | GGAACAATAGGTAATATAAAAGAACTTAAAGAAGGATATTTATCAATTGTAGTACAT | |
| | GAATTAGCTAAATTAGTAAAGGAATATAATGCAATAATTGTTCTAGAAAATTTGAAT | |
| | GCTGGATTTAAAAGGGGAAGAACTAAAGTTGAAAAACAAGTATATCAAAAATTTGA | |
| | ACTTGCATTGATAAAGAAACTTAATTATTTAGTATTTAAAAACGAAAATATTCAAAAT | |
| | AAAGGTGGTTATTTAAAAGGATTACAATTAACTCAGCCATTTGATACTTTTAAAGAT | |
| | ATTGGAAATCAATCTGGTATAATTTATTATGTTATTCCATCATATACATCGAAAATAT | |
| | GTCCTACTACAGGCTTTATAGATGTAATTAAGCCACAATATGAAAGTGTTGAAAAAG | |
| | CCAAAGAATTATTTTCTAAATTTAAGCGTATATATTTCGATAATAATAAAAAATGTTT | |
| | TATATTTGAATTTATGTATAAAGACTTTGGTAGAGATTATGGTTTAGATAAAATATGG | |
| | AGTATATGTACACTTGGAGAAAAAAGATATTATTATGATTCTAAAAATAAAGTATCAA | |
| | ATGTAATAAATGTAACAGAATCAATAATTAGTATATTACAAGAAAAAAACATAAATTA | |
| | TATAAATTCAGACAATATCATAGATGAAATTTTACAATATAGTGATGTTAAGTTGTAT | |
| | AAAGAATTATTATTTAATTTAAAAGTTGTTTTACAAATGAGATATACGAAGAGTGGTA | |
| | CAAATAATGAAGATGATTTTATTCTATCACCAGTATTAGATGAAAATGATAAGGCAT | |
| | TTTGTTCACTTAATGCAAAAGAAACAGAACCTCAAAATGCAGATGCAAACGGTGCA | |
| | TATCATATTGCTATGAAAGGTTTAAATGCAATAATGAGCATTAAGAATGGTAATGTA | |
| | GATAGAGATATTAACAATTTAGAAAATTGGATAAATTTTATACAAAAGTTTCATATAG | |
| | GTAAATAA | |
| Codon | GGCAAGTTCTTTGAAACCGACGAGTTCATCGGACAGTACAGTATCAACAAAACACT | 95 |
| optimized | GAGGTTCGAGCTCATCCCTCAAGGCAAGACCAAGGAACTGCTGAACAACTATATG | |
| coding | AACGACAACAGTAAGATCAAGCAGGACATCCTGCGGGCCGACGAGAAGAACAATT | |
| sequence (no | TCAAGGAAGTGATCGACGAGTATTATAGAGAGTTGATCCACGACGCCCTGACCGA | |
| N-terminal | CGAGGACATCTTTTCCATCACCCCTCTCGTCAAGGACGCCTACGAGCTGTACATC | |
| methionine, no | GCCTCCAGAAAAAACACCAGCGACTCCAGCAAGAAGGAGTATCGGGACGTGAAAA | |
| stop codon) | ATAAGATTAGAAAAGAGATCGCTAACATCCTGAACAAGTACAAGACAATCTACGGC | |
| | CTGGACAAGTTCGCCAATATCTACAAGTCTGAGAGCGACAAGAGCGTTGATGATG | |
| | ACGAATCTGATAACGATGACTTGGACGAGAAGAATACCACCAACGACGATAATGC | |
| | CAAGTCTGAGGACAAGCGGATCTATAAGTGGCTGAATAAGAAGCTGAGACTGAAG | |
| | CAGATCTCCAACGAAGAATACGACCGGTACTACAAGTCCCTGAACGAATACCACG | |
| | GGTTCACAACAGGACTGCAGGGCCTGCAGAACAACAAGGAAAACATGTTCAGCAG | |
| | CGAGAACAAGAGCACCGCCATCGCCTTTAGAATCATCGATGACAACATGGAAAAG | |
| | TATTTTTCTAACATCCTGCTCCTGGAGTTCATCAAAAACAAGTACAAAGATCTGTAC | |
| | GAGAAGATCGAGGAGAAGGCCAACAAGATGAACGTGGAATGCTTCACCAAGTACT | |
| | TCACCCAGGAGGGCATCGACGAGTACAATCAGATGATTGGCAGAAGCATTGAGGA | |
| | AGAATACGCCAAGGGCATCAACCAGGAGATCAACCTGTATAAGCAGAGCAAGGGT | |
| | CTAAACAATAAGGAGATCAGAACACTGAGCCCCCTGTACAAGCAGATCCTGTCCA | |
| | AGACCAGCCAGAACGAAATCATCGTGTTCAAAAACGACAAGGAAACCCTGGAATA | |
| | CATCAAGAATATCTGTGATTACATTATCGAGGAGGACATCTTCGGAAAGATGAACC | |
| | ACCTGATCAAAACCAACCTGATCGACATGTGCACCGGAATCTACATTAAGAGAAAC | |
| | GAGCTGAGCAACATCTCTTTCAAGCTCTACAACGACTGGGGCCTGCTGGACAGAA | |
| | TTATCTGTGACTACGCCAACGAGTTCAAGACAAAGAAGGAAAAGAATGAGTTCGAG | |
| | AAGCTGAACAAAGAGGTGATCTCTCTGAACCTGCTCAACGATATTTTCAACAAATA | |
| | CAAGGAAACCAGAGGCAATGATACAGACCTGAAGGAAATCGTGGAATACTTTAAAA | |
| | ACGTCGACGAGAAAATGATTGAGGACGAGTACAGCAAGATCAAGAGCATACTTAA | |
| | TCTGGAACGCATCGACATCGACCGTAGAGTGCCAAGCAAGGACGAGGAAAAGGG | |
| | CGGCGAAGGCTTTGAGCAGATCTGCATGATCAAGACGTTCCTGGATCTGCTGTTG | |
| | GAGAGCATCCACATCTACAAGCCTCTGTCTCTGATCAAGAACGGCGAGAAGGTGG | |
| | AAATCTACAATTATAACGAGAACTTCTACAACGAGTACGACATCCTGTTCAGCCAG | |
| | CTGGATAACATTATAAATCTGTACAATAAGGTGCGGAACTACTTCAGCAAGAAAAC | |
| | CTACAGCAAAGAGAAAATCAAAATCTATTTCTCCAAACCCACCCTGCTGAACGGAT | |
| | GGGACGTGAACAAGGAGATCAGCAACTACTCTATCATCCTGAGAAAAGACGAAGA | |
| | GTACTTTCTGGCAATTATGAACAGCGACAACAAGATCTTCACGAATGAGAGGCTGG | |
| | AAGAAAACTGCGCCATCACCGAGAATAATGAAGAATGTTACGAGAAAATGGTGTAC | |
| | AAGCAAATCTCTGACTCTAACAAGATGTTCAGCAAGGTGTTTTTCAGCGAGAAAAA | |
| | CAAGAAGATCTACATGCCCAGCGAAGAGATCAAGAATATCAGAAAGAACAAGACC | |
| | CATCTCAAGGTGGCCAACAATAAGGATTCTCAAACAAAGTGGATCAAGTTCATGAT | |
| | CGAGTGCTACTATAAACACCCTGAGTGGAGTAAGTACTTCGATATCAACTTCAAGA | |
| | AACCTGAAGAATATGAAAGCATCGTGGAATTTTACAACCAGGTGAACGAGAAGATC | |
| | TACAACATCAAGTTCGTGAATATCAAATGCGACTACATCAACAGCATGGTGGATTC | |
| | GGGAGAGCTGTACCTGTTCAAGATCTACAACAAGGACTTCTCTAAGAACAAGAAAA | |
| | AAAGTGGCACAGATAACCTGCACACCATGTATTGGAAGCTGCTGTTTAGCAAAGAA | |
| | AACATGAATTGCGGCGTGTACAAGCTGAACGGCCAGGCCGAGGTGTTCTTCAGAA | |
| | AGGCCAGCCTGCCTGATAAGATCACACACGAAAGAAATAAGGAGATCGACAACAA | |
| | AAATCCTATCAAGGACAAGAAAACCAGCACCTTCACATACGACCTGAAGAAAGATA | |
| | AGCGGTTCATGGAAGATAAGTTCTTCTTCCACTGCCCCATAACCATCAACTACAAG | |
| | GGCCTTAACGCCAAGGACAAGGAGATCAGAAAGTACAACGAAAAGATCAACAAAT | |
| | TCATCGCTGGCAACCCCGACATCAACATCATAGGCATCGACCGGGGCGAACGGC | |
| | ACCTGCTGTACTACACCATCATCAACCAGAAGGGAGAGATCCTGAAGCAATCTACA | |
| | CTGAACAACGTGGGCATCGAGGGCAGAGACAAAGATTACCAGGAGCTGCTGAGC | |
| | AACAAGGAAAAGGAAAGACACCTCGCTAGAAAGAGCTGGGGCACCATCGGCAAC | |
| | ATAAAAGAACTGAAGGAAGGCTACCTGAGCATCGTGGTGCACGAGCTGGCCAAGC | |
| | TCGTGAAGGAGTACAACGCCATCATCGTGCTGGAGAATCTGAACGCCGGCTTCAA | |
| | GAGAGGCAGAACCAAGGTGGAAAAACAGGTCTACCAGAAGTTTGAGCTGGCCCT | |
| | GATCAAGAAGCTGAACTACCTCGTGTTCAAGAACGAGAACATCCAGAACAAGGGA | |
| | GGCTACCTGAAGGGACTGCAACTGACACAGCCTTTCGACACCTTTAAGGATATCG | |
| | GCAACCAGAGCGGCATCATCTACTACGTGATCCCCAGCTACACAAGCAAAATTTGT | |
| | CCAACAACCGGCTTCATCGACGTGATCAAACCTCAGTACGAGTCTGTGGAAAAGG | |
| | CCAAGGAGCTGTTCTCCAAATTCAAACGGATTTACTTCGACAACAACAAGAAGTGC | |
| | TTTATCTTCGAATTTATGTACAAAGATTTCGGCAGAGATTACGGTCTGGACAAGATC | |
| | TGGAGCATCTGTACCCTGGGCGAGAAGAGATACTACTACGACAGCAAGAACAAGG | |
| | TTTCCAATGTGATCAACGTGACCGAGAGCATCATCAGCATCCTGCAGGAGAAGAA | |
| | CATCAACTACATCAACAGCGACAACATCATCGACGAGATCCTGCAGTACAGCGAC | |
| | GTGAAGCTGTATAAGGAGCTGCTTTTTAACCTGAAGGTGGTGCTGCAGATGCGGT | |
| | ACACCAAGAGCGGCACCAATAACGAGGACGACTTCATTCTGTCTCCTGTGCTGGA | |
| | CGAGAACGACAAGGCCTTCTGCAGCCTGAACGCTAAGGAAACAGAGCCTCAGAAT | |
| | GCTGATGCTAATGGCGCCTATCATATCGCCATGAAGGGACTGAACGCCATCATGT | |
| | CCATCAAGAACGGCAACGTGGATAGAGATATTAACAACCTGGAAAACTGGATCAA | |
| | CTTCATCCAGAAATTCCACATCGGGAAG | |
| Expression | ATGggctccggaGGCAAGTTCTTTGAAACCGACGAGTTCATCGGACAGTACAGTATCA | 96 |
| construct (with | ACAAAACACTGAGGTTCGAGCTCATCCCTCAAGGCAAGACCAAGGAACTGCTGAA | |
| N-terminal | CAACTATATGAACGACAACAGTAAGATCAAGCAGGACATCCTGCGGGCCGACGAG | |
| methionine | AAGAACAATTTCAAGGAAGTGATCGACGAGTATTATAGAGAGTTGATCCACGACGC | |
| and stop | CCTGACCGACGAGGACATCTTTTCCATCACCCCTCTCGTCAAGGACGCCTACGAG | |
| codon, | CTGTACATCGCCTCCAGAAAAAACACCAGCGACTCCAGCAAGAAGGAGTATCGGG | |
| includes V5- | ACGTGAAAAATAAGATTAGAAAAGAGATCGCTAACATCCTGAACAAGTACAAGACA | |
| tag and C- | ATCTACGGCCTGGACAAGTTCGCCAATATCTACAAGTCTGAGAGCGACAAGAGCG | |
| terminal NLS) | TTGATGATGACGAATCTGATAACGATGACTTGGACGAGAAGAATACCACCAACGAC | |
| | GATAATGCCAAGTCTGAGGACAAGCGGATCTATAAGTGGCTGAATAAGAAGCTGA | |
| | GACTGAAGCAGATCTCCAACGAAGAATACGACCGGTACTACAAGTCCCTGAACGA | |
| | ATACCACGGGTTCACAACAGGACTGCAGGGCCTGCAGAACAACAAGGAAAACATG | |
| | TTCAGCAGCGAGAACAAGAGCACCGCCATCGCCTTTAGAATCATCGATGACAACA | |
| | TGGAAAAGTATTTTTCTAACATCCTGCTCCTGGAGTTCATCAAAAACAAGTACAAAG | |
| | ATCTGTACGAGAAGATCGAGGAGAAGGCCAACAAGATGAACGTGGAATGCTTCAC | |
| | CAAGTACTTCACCCAGGAGGGCATCGACGAGTACAATCAGATGATTGGCAGAAGC | |
| | ATTGAGGAAGAATACGCCAAGGGCATCAACCAGGAGATCAACCTGTATAAGCAGA | |
| | GCAAGGGTCTAAACAATAAGGAGATCAGAACACTGAGCCCCCTGTACAAGCAGAT | |
| | CCTGTCCAAGACCAGCCAGAACGAAATCATCGTGTTCAAAAACGACAAGGAAACC | |
| | CTGGAATACATCAAGAATATCTGTGATTACATTATCGAGGAGGACATCTTCGGAAA | |
| | GATGAACCACCTGATCAAAACCAACCTGATCGACATGTGCACCGGAATCTACATTA | |
| | AGAGAAACGAGCTGAGCAACATCTCTTTCAAGCTCTACAACGACTGGGGCCTGCT | |
| | GGACAGAATTATCTGTGACTACGCCAACGAGTTCAAGACAAAGAAGGAAAAGAAT | |
| | GAGTTCGAGAAGCTGAACAAAGAGGTGATCTCTCTGAACCTGCTCAACGATATTTT | |
| | CAACAAATACAAGGAAACCAGAGGCAATGATACAGACCTGAAGGAAATCGTGGAA | |
| | TACTTTAAAAACGTCGACGAGAAAATGATTGAGGACGAGTACAGCAAGATCAAGAG | |
| | CATACTTAATCTGGAACGCATCGACATCGACCGTAGAGTGCCAAGCAAGGACGAG | |
| | GAAAAGGGCGGCGAAGGCTTTGAGCAGATCTGCATGATCAAGACGTTCCTGGATC | |
| | TGCTGTTGGAGAGCATCCACATCTACAAGCCTCTGTCTCTGATCAAGAACGGCGA | |
| | GAAGGTGGAAATCTACAATTATAACGAGAACTTCTACAACGAGTACGACATCCTGT | |
| | TCAGCCAGCTGGATAACATTATAAATCTGTACAATAAGGTGCGGAACTACTTCAGC | |
| | AAGAAAACCTACAGCAAAGAGAAAATCAAAATCTATTTCTCCAAACCCACCCTGCT | |
| | GAACGGATGGGACGTGAACAAGGAGATCAGCAACTACTCTATCATCCTGAGAAAA | |
| | GACGAAGAGTACTTTCTGGCAATTATGAACAGCGACAACAAGATCTTCACGAATGA | |
| | GAGGCTGGAAGAAAACTGCGCCATCACCGAGAATAATGAAGAATGTTACGAGAAA | |
| | ATGGTGTACAAGCAAATCTCTGACTCTAACAAGATGTTCAGCAAGGTGTTTTTCAG | |
| | CGAGAAAAACAAGAAGATCTACATGCCCAGCGAAGAGATCAAGAATATCAGAAAG | |
| | AACAAGACCCATCTCAAGGTGGCCAACAATAAGGATTCTCAAACAAAGTGGATCAA | |
| | GTTCATGATCGAGTGCTACTATAAACACCCTGAGTGGAGTAAGTACTTCGATATCA | |
| | ACTTCAAGAAACCTGAAGAATATGAAAGCATCGTGGAATTTTACAACCAGGTGAAC | |
| | GAGAAGATCTACAACATCAAGTTCGTGAATATCAAATGCGACTACATCAACAGCAT | |
| | GGTGGATTCGGGAGAGCTGTACCTGTTCAAGATCTACAACAAGGACTTCTCTAAG | |
| | AACAAGAAAAAAAGTGGCACAGATAACCTGCACACCATGTATTGGAAGCTGCTGTT | |
| | TAGCAAAGAAAACATGAATTGCGGCGTGTACAAGCTGAACGGCCAGGCCGAGGT | |
| | GTTCTTCAGAAAGGCCAGCCTGCCTGATAAGATCACACACGAAAGAAATAAGGAG | |
| | ATCGACAACAAAAATCCTATCAAGGACAAGAAAACCAGCACCTTCACATACGACCT | |
| | GAAGAAAGATAAGCGGTTCATGGAAGATAAGTTCTTCTTCCACTGCCCCATAACCA | |
| | TCAACTACAAGGGCCTTAACGCCAAGGACAAGGAGATCAGAAAGTACAACGAAAA | |
| | GATCAACAAATTCATCGCTGGCAACCCCGACATCAACATCATAGGCATCGACCGG | |
| | GGCGAACGGCACCTGCTGTACTACACCATCATCAACCAGAAGGGAGAGATCCTGA | |
| | AGCAATCTACACTGAACAACGTGGGCATCGAGGGCAGAGACAAAGATTACCAGGA | |
| | GCTGCTGAGCAACAAGGAAAAGGAAAGACACCTCGCTAGAAAGAGCTGGGGCAC | |
| | CATCGGCAACATAAAAGAACTGAAGGAAGGCTACCTGAGCATCGTGGTGCACGAG | |
| | CTGGCCAAGCTCGTGAAGGAGTACAACGCCATCATCGTGCTGGAGAATCTGAACG | |
| | CCGGCTTCAAGAGAGGCAGAACCAAGGTGGAAAAACAGGTCTACCAGAAGTTTGA | |
| | GCTGGCCCTGATCAAGAAGCTGAACTACCTCGTGTTCAAGAACGAGAACATCCAG | |
| | AACAAGGGAGGCTACCTGAAGGGACTGCAACTGACACAGCCTTTCGACACCTTTA | |
| | AGGATATCGGCAACCAGAGCGGCATCATCTACTACGTGATCCCCAGCTACACAAG | |
| | CAAAATTTGTCCAACAACCGGCTTCATCGACGTGATCAAACCTCAGTACGAGTCTG | |
| | TGGAAAAGGCCAAGGAGCTGTTCTCCAAATTCAAACGGATTTACTTCGACAACAAC | |
| | AAGAAGTGCTTTATCTTCGAATTTATGTACAAAGATTTCGGCAGAGATTACGGTCT | |
| | GGACAAGATCTGGAGCATCTGTACCCTGGGCGAGAAGAGATACTACTACGACAGC | |
| | AAGAACAAGGTTTCCAATGTGATCAACGTGACCGAGAGCATCATCAGCATCCTGC | |
| | AGGAGAAGAACATCAACTACATCAACAGCGACAACATCATCGACGAGATCCTGCA | |
| | GTACAGCGACGTGAAGCTGTATAAGGAGCTGCTTTTTAACCTGAAGGTGGTGCTG | |
| | CAGATGCGGTACACCAAGAGCGGCACCAATAACGAGGACGACTTCATTCTGTCTC | |
| | CTGTGCTGGACGAGAACGACAAGGCCTTCTGCAGCCTGAACGCTAAGGAAACAGA | |
| | GCCTCAGAATGCTGATGCTAATGGCGCCTATCATATCGCCATGAAGGGACTGAAC | |
| | GCCATCATGTCCATCAAGAACGGCAACGTGGATAGAGATATTAACAACCTGGAAAA | |
| | CTGGATCAACTTCATCCAGAAATTCCACATCGGGAAGtctagaAAGCGGACAGCAGA | |
| | CGGCTCCGAATTTGAAAGCCCTAAGAAAAAGAGAAAGGTGggatccGGCAAACCTAT | |
| | CCCCAATCCCCTGCTGGGCCTGGACAGCACCTGA | |

In some embodiments a ZSYN Type V Cas protein comprises an amino acid sequence of SEQ ID NO: 91, SEQ ID NO:92, or SEQ ID NO:93. In some embodiments, a ZSYN Type V Cas protein has nickase activity, for example resulting from one or more amino acid substitutions relative to the sequence of SEQ ID NO:91, SEQ ID NO: 92, or SEQ ID NO:93. In some embodiments, the one or more amino acid substitutions providing nickase activity comprise a D902 substitution, wherein the position of the D902 substitution is defined with respect to the amino acid numbering of SEQ ID NO:92 (corresponding to amino acid 908 of SEQ ID NO: 121). In some embodiments, the one or more amino acid substitutions providing nickase activity comprise an E991 substitution, wherein the position of the E991 substitution is defined with respect to the amino acid numbering of SEQ ID NO:92 (corresponding to amino acid 993 of SEQ ID NO: 121). In some embodiments, the one or more amino acid substitutions providing nickase activity comprise a R1200 substitution, wherein the position of the R1200 substitution is defined with respect to the amino acid numbering of SEQ ID NO:92 (corresponding to amino acid 1226 of SEQ ID NO: 121). In some embodiments, the one or more amino acid substitutions providing nickase activity comprise a D1239 substitution, wherein the position of the D1239 substitution is defined with respect to the amino acid numbering of SEQ ID NO:92 (corresponding to amino acid 1263 of SEQ ID NO:121). In some embodiments, a ZSYN Type V Cas protein is catalytically inactive, for example due to a R1200 substitution in combination with a D902 substitution, a E991 substitution, and/or D1239 substitution.

### 6.2.17. ZRBH Type V Cas Protein

In one aspect, the disclosure provides ZRBH Type V Cas proteins. ZRBH Type V Cas proteins can be further classified as Type V-A Cas proteins. The ZRBH Type V Cas proteins typically comprise an amino acid sequence that is at least 50%, at least 55%, at least 60%, at least 65%, at least 70%, at least 75%, at least 85%, at least 90%, or at least 95% identical to SEQ ID NO:97. In some embodiments, the ZRBH Type V Cas proteins comprise an amino acid sequence that is at least 95%, at least 96%, at least 97%, at least 98%, or at least 99% identical to SEQ ID NO:97. In some embodiments, a ZRBH Type V Cas protein comprises an amino acid sequence that is identical to SEQ ID NO:97.

Exemplary ZRBH Type V Cas protein sequences and nucleotide sequences encoding exemplary ZRBH Type V Cas proteins are set forth in Table 1Q.

TABLE 1Q
ZRBH Type V Cas Sequences

| | | SEQ |
|---|---|---|
| | | ID |
| Name | Sequence | NO. |
| Wildtype | EFDNSFVNRYPLSKTLSFSLLPVGSTEANFEKKLLLQEDEKRAAEYILVKSYIDRYHKAY | 97 |
| amino acid | IESVLSKVVLDGINNYAQLYCKNNKTEQDIKRLEQLEGSFRKQISKSLKSDARYKLIYKK | |
| sequence | EMLEKLLPEFLDNEEEKARVISFENFTTYFTGFHTNRENMYTDEAKSTAVSFRCINDNL | |
| (without N- | PKFLDNISVFKWVTAFLSESDINELKADFSGLLGCSLEEMFTPDYFSFVLSQSGIERYN | |
| terminal | NVIGGYTCSDGEKVKGLNEYINLYNQKLQHGEKKLPLLKRLFKQILSDTESVSFIPEKLE | |
| methionine) | NDDAVISAINGFCNIKIENETFFEILDKTKCLFSNLNEFDSAGVYITNGFAVTDISNAVFG | |
| | TWDVISEAWKKEYAKAIPLKNIAKADAYYEKQGKAYKAIKSFSVSELQRLANTTEGKAA | |
| | YKHNGDISAYFSETVCFAVQDIFEKYSSSKALFASPYKNEKRLFKNNEAIALIKDFLDSIK | |
| | NLEKLIKPFNGSGRENDKDESFYGEFTACYERLSKIDLLYDKVRNYMTQKPYSGDKIKL | |
| | NFENPQFLNGWDRNKERDYRTVLLRKGGYYYLAIMDKSNNRIFEDLPEPKNGEDCYE | |
| | KIDYKLLPGPNKMLPKVFFAASNIDYFAPSEQILKIRQKETFKKGVNFNIDDCHAFIDFLK | |
| | ESIEKHDEWCKYGFEFKDTSDYNNIGEFYKDVREQGYSISFRNVPESYINSCVNSGSL | |
| | YLFQIYNKDFSPYSKGTKSLHTLYFEMLFDERNLKNVVYQLNGGAEMFYRKASIKERD | |
| | KIVHPANIPIKNKNPDNPKAESVFEYDIIKDRRFTERQFSLHIPVTLNFKGSGGSANLNA | |
| | DVRRAIRGADENYVIGIDRGERNLLYITVINSKGEIVEQIPGNVIINGKQVVDYHKLLDAK | |
| | EKERLAARQNWTTVENIKELKEGYLSVIIHNICELVKKYNAVIAMEDLSSGFKNSRVKVE | |
| | KQVYQKFEKMLTEKLNFLVDKKADVQSRGGLLQAYQLTNSTKDYKRAGSQDGIVFYV | |
| | PAWLTSKIDPVTGFVDLLKPKYTSVQEAKELFSNFEAVEYIPEEDLFSFTFDYSKFPRC | |
| | SVAYRNKWTVYSNGERIYTFRDKNSNNEYVSKTVALTTEFKSLFDEYSVYYRDNLKSQ | |
| | ILCQDKVDFFKQLIRLLSLTMQMRNSISNSAVDYLISPVKDKNGNFFDSRKSIKNLPENA | |
| | DANGAYNIAKKALWAIGQIKEADENDLMKVKLSVSNKEWLKYVQEVE | |
| Wildtype | MEFDNSFVNRYPLSKTLSFSLLPVGSTEANFEKKLLLQEDEKRAAEYILVKSYIDRYHK | 98 |
| amino acid | AYIESVLSKVVLDGINNYAQLYCKNNKTEQDIKRLEQLEGSFRKQISKSLKSDARYKLIY | |
| sequence (with | KKEMLEKLLPEFLDNEEEKARVISFENFTTYFTGFHTNRENMYTDEAKSTAVSFRCIND | |
| N-terminal | NLPKFLDNISVFKWVTAFLSESDINELKADFSGLLGCSLEEMFTPDYFSFVLSQSGIER | |
| methionine) | YNNVIGGYTCSDGEKVKGLNEYINLYNQKLQHGEKKLPLLKRLFKQILSDTESVSFIPEK | |
| | LENDDAVISAINGFCNIKIENETFFEILDKTKCLFSNLNEFDSAGVYITNGFAVTDISNAVF | |
| | GTWDVISEAWKKEYAKAIPLKNIAKADAYYEKQGKAYKAIKSFSVSELQRLANTTEGKA | |
| | AYKHNGDISAYFSETVCFAVQDIFEKYSSSKALFASPYKNEKRLFKNNEAIALIKDFLDSI | |
| | KNLEKLIKPFNGSGRENDKDESFYGEFTACYERLSKIDLLYDKVRNYMTQKPYSGDKIK | |
| | LNFENPQFLNGWDRNKERDYRTVLLRKGGYYYLAIMDKSNNRIFEDLPEPKNGEDCY | |
| | EKIDYKLLPGPNKMLPKVFFAASNIDYFAPSEQILKIRQKETFKKGVNFNIDDCHAFIDFL | |
| | KESIEKHDEWCKYGFEFKDTSDYNNIGEFYKDVREQGYSISFRNVPESYINSCVNSGS | |
| | LYLFQIYNKDFSPYSKGTKSLHTLYFEMLFDERNLKNVVYQLNGGAEMFYRKASIKER | |
| | DKIVHPANIPIKNKNPDNPKAESVFEYDIIKDRRFTERQFSLHIPVTLNFKGSGGSANLN | |
| | ADVRRAIRGADENYVIGIDRGERNLLYITVINSKGEIVEQIPGNVIINGKQVVDYHKLLDA | |
| | KEKERLAARQNWTTVENIKELKEGYLSVIIHNICELVKKYNAVIAMEDLSSGFKNSRVKV | |
| | EKQVYQKFEKMLTEKLNFLVDKKADVQSRGGLLQAYQLTNSTKDYKRAGSQDGIVFY | |
| | VPAWLTSKIDPVTGFVDLLKPKYTSVQEAKELFSNFEAVEYIPEEDLFSFTFDYSKFPR | |
| | CSVAYRNKWTVYSNGERIYTFRDKNSNNEYVSKTVALTTEFKSLFDEYSVYYRDNLKS | |
| | QILCQDKVDFFKQLIRLLSLTMQMRNSISNSAVDYLISPVKDKNGNFFDSRKSIKNLPEN | |
| | ADANGAYNIAKKALWAIGQIKEADENDLMKVKLSVSNKEWLKYVQEVE | |
| Expression | MGSGEFDNSFVNRYPLSKTLSFSLLPVGSTEANFEKKLLLQEDEKRAAEYILVKSYIDR | 99 |
| construct (with | YHKAYIESVLSKVVLDGINNYAQLYCKNNKTEQDIKRLEQLEGSFRKQISKSLKSDARY | |
| N-terminal | KLIYKKEMLEKLLPEFLDNEEEKARVISFENFTTYFTGFHTNRENMYTDEAKSTAVSFR | |
| methionine, | CINDNLPKFLDNISVFKWVTAFLSESDINELKADFSGLLGCSLEEMFTPDYFSFVLSQS | |
| V5-tag and C- | GIERYNNVIGGYTCSDGEKVKGLNEYINLYNQKLQHGEKKLPLLKRLFKQILSDTESVS | |
| terminal NLS) | FIPEKLENDDAVISAINGFCNIKIENETFFEILDKTKCLFSNLNEFDSAGVYITNGFAVTDI | |
| aa sequence | SNAVFGTWDVISEAWKKEYAKAIPLKNIAKADAYYEKQGKAYKAIKSFSVSELQRLANT | |
| | TEGKAAYKHNGDISAYFSETVCFAVQDIFEKYSSSKALFASPYKNEKRLFKNNEAIALIK | |
| | DFLDSIKNLEKLIKPFNGSGRENDKDESFYGEFTACYERLSKIDLLYDKVRNYMTQKPY | |
| | SGDKIKLNFENPQFLNGWDRNKERDYRTVLLRKGGYYYLAIMDKSNNRIFEDLPEPKN | |
| | GEDCYEKIDYKLLPGPNKMLPKVFFAASNIDYFAPSEQILKIRQKETFKKGVNFNIDDCH | |
| | AFIDFLKESIEKHDEWCKYGFEFKDTSDYNNIGEFYKDVREQGYSISFRNVPESYINSC | |
| | VNSGSLYLFQIYNKDFSPYSKGTKSLHTLYFEMLFDERNLKNVVYQLNGGAEMFYRKA | |
| | SIKERDKIVHPANIPIKNKNPDNPKAESVFEYDIIKDRRFTERQFSLHIPVTLNFKGSGGS | |
| | ANLNADVRRAIRGADENYVIGIDRGERNLLYITVINSKGEIVEQIPGNVIINGKQVVDYHK | |
| | LLDAKEKERLAARQNWTTVENIKELKEGYLSVIIHNICELVKKYNAVIAMEDLSSGFKNS | |
| | RVKVEKQVYQKFEKMLTEKLNFLVDKKADVQSRGGLLQAYQLTNSTKDYKRAGSQD | |
| | GIVFYVPAWLTSKIDPVTGFVDLLKPKYTSVQEAKELFSNFEAVEYIPEEDLFSFTFDYS | |
| | KFPRCSVAYRNKWTVYSNGERIYTFRDKNSNNEYVSKTVALTTEFKSLFDEYSVYYRD | |
| | NLKSQILCQDKVDFFKQLIRLLSLTMQMRNSISNSAVDYLISPVKDKNGNFFDSRKSIKN | |
| | LPENADANGAYNIAKKALWAIGQIKEADENDLMKVKLSVSNKEWLKYVQEVESRKRTA | |
| | DGSEFESPKKKRKVGSGKPIPNPLLGLDST | |
| Wildtype | ATGGAATTCGACAATAGCTTTGTTAACCGATACCCTTTATCAAAAACACTAAGCTTC | 100 |
| coding | AGTTTGCTTCCTGTTGGCAGTACCGAAGCAAATTTTGAGAAAAAACTGTTGCTGCA | |
| sequence (with | GGAGGACGAAAAAAGAGCCGCGGAATATATTTTGGTGAAGTCATACATTGACAGA | |
| N-terminal | TACCATAAAGCCTATATTGAATCGGTTTTATCAAAGGTTGTGCTTGACGGCATAAAT | |
| methionine | AACTATGCACAGCTGTACTGCAAGAACAACAAAACCGAACAGGATATCAAACGACT | |
| and stop | GGAGCAGCTTGAAGGTTCATTTAGAAAGCAGATTTCAAAGAGCTTGAAATCCGATG | |
| codon) | CCCGTTATAAGTTGATTTATAAAAAAGAAATGCTTGAAAAGCTTTTGCCTGAGTTTC | |
| | TTGATAATGAAGAAGAAAAGGCGAGGGTAATATCTTTTGAAAACTTTACAACATATT | |
| | TCACAGGCTTTCATACCAATAGAGAAAATATGTATACCGACGAAGCAAAATCCACT | |
| | GCGGTGTCCTTCAGATGTATAAATGATAATTTACCAAAATTTCTTGATAATATTTCA | |
| | GTTTTTAAATGGGTTACGGCATTTTTGAGCGAAAGTGATATCAACGAATTAAAGGC | |
| | GGATTTTTCAGGTCTGTTAGGTTGTTCGCTTGAAGAAATGTTTACACCGGATTATTT | |
| | TTCCTTTGTGTTATCTCAAAGCGGGATAGAAAGATATAACAATGTTATCGGCGGTT | |
| | ACACATGTTCTGACGGTGAAAAAGTTAAGGGACTAAATGAATACATAAATTTATACA | |
| | ACCAAAAGTTACAACACGGTGAAAAAAAGCTCCCGCTTTTAAAACGCTTGTTCAAG | |
| | CAGATATTGAGTGATACCGAAAGTGTATCCTTTATTCCGGAAAAGCTTGAAAACGA | |
| | CGATGCTGTTATTTCTGCGATAAACGGATTTTGTAATATCAAAATTGAAAACGAAAC | |
| | ATTCTTTGAAATTCTTGATAAAACTAAATGCTTGTTTTCAAATTTAAATGAGTTTGAC | |
| | AGCGCCGGTGTATATATTACCAACGGTTTTGCTGTAACCGATATTTCAAATGCTGT | |
| | TTTCGGTACTTGGGATGTTATTTCGGAAGCGTGGAAAAAGGAGTATGCGAAAGCA | |
| | ATCCCGCTTAAAAATATCGCCAAGGCAGATGCATATTACGAAAAGCAGGGCAAGG | |
| | CGTATAAGGCAATTAAAAGCTTTTCGGTAAGCGAGCTTCAAAGGCTGGCCAACACA | |
| | ACAGAAGGGAAGGCGGCATATAAGCACAACGGAGATATTTCTGCATATTTTTCGGA | |
| | AACTGTTTGCTTTGCGGTTCAAGATATATTTGAAAAATACAGTAGTTCAAAAGCCCT | |
| | TTTTGCGTCGCCCTATAAAAATGAAAAGCGGCTCTTCAAAAACAATGAGGCTATAG | |
| | CGCTGATTAAGGATTTTCTTGACAGCATCAAAAATCTGGAAAAGCTTATTAAACCAT | |
| | TTAACGGCTCCGGTAGAGAAAACGATAAGGACGAAAGCTTCTACGGTGAATTTAC | |
| | CGCTTGCTACGAGAGGCTTTCTAAAATTGACCTGCTATATGATAAGGTTCGCAACT | |
| | ATATGACACAAAAACCTTATTCCGGGGACAAGATAAAGTTGAATTTTGAAAATCCG | |
| | CAATTTCTAAATGGTTGGGACAGGAACAAAGAGCGGGATTACAGAACTGTTCTCTT | |
| | AAGAAAAGGCGGGTATTACTACCTTGCTATTATGGATAAAAGCAACAACAGGATTT | |
| | TTGAAGATTTGCCGGAGCCCAAAAACGGCGAGGATTGTTATGAAAAAATAGACTAC | |
| | AAGCTTCTGCCGGGACCGAATAAGATGTTGCCAAAGGTGTTTTTTGCCGCGAGCA | |
| | ATATTGATTATTTTGCACCCTCTGAGCAAATTTTGAAAATTAGACAGAAAGAAACCT | |
| | TTAAGAAGGGTGTGAATTTTAATATTGATGATTGCCATGCTTTCATAGACTTCCTTA | |
| | AAGAGTCTATAGAAAAACACGATGAGTGGTGCAAGTATGGGTTCGAATTTAAAGAT | |
| | ACTTCAGATTATAACAACATCGGTGAATTTTATAAAGATGTAAGGGAGCAGGGCTA | |
| | TTCTATCAGCTTTAGAAATGTGCCTGAGTCTTATATAAATTCTTGCGTTAATTCCGG | |
| | TTCACTTTACCTTTTCCAAATCTACAACAAGGATTTTTCACCTTACAGCAAAGGGAC | |
| | CAAGAGTTTGCACACATTGTATTTTGAAATGCTTTTTGATGAAAGGAACCTTAAGAA | |
| | TGTTGTTTATCAGCTTAACGGCGGTGCAGAGATGTTTTACCGCAAAGCAAGTATTA | |
| | AGGAAAGGGATAAAATAGTACACCCTGCTAATATTCCGATAAAAAATAAAAATCCC | |
| | GATAACCCAAAAGCTGAAAGTGTTTTTGAGTATGACATCATAAAGGACAGACGCTT | |
| | TACTGAAAGACAGTTCTCTTTGCATATTCCTGTTACGCTCAATTTTAAAGGCTCGGG | |
| | CGGCTCTGCAAATCTTAATGCTGATGTGCGCAGAGCCATAAGAGGCGCTGATGAA | |
| | AACTATGTTATAGGTATAGACAGAGGAGAAAGAAATTTGCTTTACATCACCGTAATA | |
| | AACAGTAAAGGTGAAATTGTTGAGCAGATTCCGGGCAATGTAATAATCAACGGAAA | |
| | ACAAGTGGTCGATTATCACAAGCTGCTTGATGCCAAAGAAAAAGAGCGTCTTGCA | |
| | GCACGGCAAAACTGGACAACGGTTGAAAATATCAAGGAGCTTAAAGAGGGCTATT | |
| | TGAGCGTAATCATACACAATATTTGTGAACTTGTAAAAAAATACAATGCTGTTATTG | |
| | CTATGGAGGATCTTTCTTCCGGTTTTAAAAACAGCAGGGTTAAAGTAGAAAAACAG | |
| | GTTTATCAGAAATTTGAAAAAATGCTTACCGAAAAGCTTAATTTTCTTGTTGATAAAA | |
| | AAGCTGATGTTCAAAGCAGGGGAGGACTTCTGCAGGCATATCAGTTAACAAACAG | |
| | CACCAAGGATTATAAGCGGGCAGGCTCACAAGACGGTATTGTTTTCTATGTTCCG | |
| | GCGTGGCTTACAAGCAAAATCGATCCCGTTACGGGTTTTGTTGATTTGCTTAAGCC | |
| | TAAGTATACAAGTGTGCAGGAAGCAAAGGAGCTGTTTTCAAATTTTGAAGCTGTTG | |
| | AATATATCCCTGAGGAGGATTTGTTCAGCTTTACTTTTGATTATTCGAAATTTCCCC | |
| | GTTGCTCCGTAGCTTACCGTAACAAATGGACTGTATACTCAAACGGCGAAAGAATT | |
| | TATACATTCAGGGATAAAAACAGCAATAATGAATATGTTAGCAAAACAGTTGCTCTT | |
| | ACAACGGAGTTTAAATCCTTGTTTGACGAATACAGCGTTTATTACCGCGATAACCTT | |
| | AAATCGCAGATTCTATGTCAAGATAAAGTCGATTTCTTCAAACAGCTAATTCGGTTA | |
| | CTGTCTTTGACAATGCAAATGCGAAACAGTATTTCAAATTCAGCAGTAGATTATCTG | |
| | ATTTCTCCGGTTAAGGATAAAAACGGAAATTTCTTTGACAGCCGGAAAAGTATAAA | |
| | AAATCTTCCGGAAAATGCAGATGCTAACGGTGCTTACAACATTGCCAAAAAGGCTC | |
| | TTTGGGCAATCGGGCAAATAAAGGAAGCGGATGAGAATGATTTAATGAAGGTCAA | |
| | GCTGTCTGTTTCAAACAAGGAATGGCTTAAATATGTGCAGGAGGTAGAATGA | |
| Codon | GAATTTGATAACTCTTTCGTGAATAGATATCCTCTGAGCAAGACCCTGAGCTTCAG | 101 |
| optimized | TCTGCTGCCAGTGGGCAGCACCGAAGCCAACTTCGAAAAAAAGCTGCTGCTGCAG | |
| coding | GAGGACGAAAAGAGAGCCGCCGAGTACATCCTGGTGAAAAGCTACATCGACAGAT | |
| sequence (no | ACCACAAGGCCTACATCGAGAGCGTGCTGAGCAAGGTGGTGCTGGACGGCATCA | |
| N-terminal | ACAACTATGCCCAGCTGTACTGCAAGAACAACAAGACCGAACAGGACATCAAGCG | |
| methionine, no | GCTGGAGCAGCTGGAGGGCAGCTTCAGAAAGCAGATCTCTAAAAGCCTGAAGTCC | |
| stop codon) | GACGCCAGATACAAGCTGATCTACAAAAAGGAGATGCTGGAAAAGCTCCTGCCTG | |
| | AGTTCCTGGACAACGAGGAAGAAAAGGCTAGAGTGATCAGCTTCGAGAACTTTAC | |
| | AACCTACTTCACTGGCTTCCACACCAACCGGGAAAACATGTACACCGATGAGGCC | |
| | AAGTCTACGGCCGTTTCCTTTAGGTGTATCAACGATAACCTGCCAAAGTTCCTGGA | |
| | CAACATCAGCGTATTCAAGTGGGTCACCGCCTTTCTGAGCGAGTCTGACATCAAC | |
| | GAACTGAAGGCCGATTTCAGCGGCCTGTTGGGCTGCTCCCTGGAAGAGATGTTCA | |
| | CCCCTGATTACTTCAGCTTCGTGCTGTCTCAGAGCGGCATCGAGAGATACAACAA | |
| | CGTGATCGGCGGATACACCTGTAGCGATGGCGAGAAAGTCAAAGGACTTAATGAG | |
| | TACATCAACCTGTATAACCAGAAGCTGCAACACGGCGAAAAGAAACTGCCCCTGC | |
| | TCAAGCGGCTGTTCAAGCAGATTCTGTCAGACACCGAGAGCGTGTCCTTCATCCC | |
| | CGAGAAACTGGAAAATGATGACGCCGTGATCTCCGCCATTAACGGATTTTGTAATA | |
| | TCAAGATCGAGAATGAAACATTCTTCGAGATCCTGGACAAGACCAAGTGCCTGTTC | |
| | AGCAATCTGAACGAGTTCGACTCTGCCGGAGTGTACATCACCAACGGCTTCGCAG | |
| | TGACAGACATCAGCAACGCCGTGTTCGGCACCTGGGACGTCATCAGCGAAGCCT | |
| | GGAAGAAAGAGTACGCCAAAGCTATCCCCCTGAAGAACATCGCTAAGGCCGACGC | |
| | CTACTATGAGAAGCAGGGCAAGGCCTACAAGGCCATCAAGAGCTTCTCTGTAAGC | |
| | GAACTGCAGAGACTGGCCAACACCACGGAGGGAAAGGCCGCCTACAAGCACAAC | |
| | GGCGACATCAGCGCCTATTTCAGCGAGACAGTCTGCTTCGCTGTGCAGGATATCT | |
| | TCGAGAAGTATAGCAGCAGCAAGGCCCTGTTCGCCAGCCCCTATAAGAACGAGAA | |
| | GCGGCTGTTCAAGAACAATGAGGCAATCGCTCTGATTAAGGACTTCCTGGATAGC | |
| | ATCAAGAACCTGGAGAAGCTGATTAAGCCATTCAACGGCAGCGGCAGAGAGAACG | |
| | ACAAGGACGAGAGCTTTTACGGCGAGTTCACCGCCTGCTACGAGCGGCTGAGCA | |
| | AAATCGATCTGCTGTACGACAAGGTGCGGAACTACATGACACAGAAACCTTACAG | |
| | CGGCGATAAGATCAAGCTGAACTTCGAGAATCCTCAGTTCCTGAACGGATGGGAT | |
| | AGAAACAAGGAGCGGGATTACAGAACAGTGCTGCTGAGAAAGGGAGGTTATTACT | |
| | ACCTGGCCATCATGGACAAGAGCAACAACCGGATCTTCGAGGATCTGCCTGAGCC | |
| | TAAGAATGGTGAGGACTGCTACGAAAAAATCGATTACAAGCTGCTGCCTGGCCCT | |
| | AACAAGATGCTGCCCAAAGTGTTCTTCGCCGCTAGTAACATCGACTACTTCGCCCC | |
| | TAGCGAACAGATCCTCAAAATCCGGCAGAAGGAAACCTTCAAAAAGGGCGTGAAC | |
| | TTCAACATTGACGACTGTCACGCCTTCATCGACTTCCTGAAGGAATCTATCGAGAA | |
| | GCACGACGAGTGGTGCAAGTACGGCTTCGAGTTTAAGGACACCAGCGACTACAAC | |
| | AATATAGGCGAGTTCTACAAGGACGTGCGGGAACAGGGCTACAGCATCTCTTTTC | |
| | GGAATGTGCCCGAGTCCTACATCAACAGCTGCGTGAACTCTGGCTCTCTGTACCT | |
| | GTTTCAGATCTACAACAAAGATTTTAGCCCTTACTCTAAGGGCACAAAGAGCCTGC | |
| | ACACCCTGTACTTTGAAATGCTGTTTGACGAGCGCAACCTGAAGAACGTGGTGTAT | |
| | CAGCTGAATGGTGGCGCTGAGATGTTCTACAGAAAGGCCAGCATCAAGGAAAGAG | |
| | ACAAGATCGTGCACCCCGCCAACATCCCTATCAAGAACAAGAACCCCGACAACCC | |
| | TAAGGCCGAGAGCGTGTTCGAATACGACATTATCAAGGACAGAAGATTCACCGAA | |
| | CGGCAGTTCTCCCTGCACATCCCTGTGACCCTGAACTTCAAAGGCTCTGGCGGAT | |
| | CTGCCAACCTGAACGCCGACGTTAGGCGGGCTATCAGAGGCGCCGATGAGAACT | |
| | ACGTGATCGGCATCGACCGGGGCGAGAGGAACCTGCTGTACATCACAGTGATCA | |
| | ATAGCAAGGGCGAGATCGTGGAACAAATCCCAGGCAACGTGATCATCAACGGCAA | |
| | GCAAGTGGTGGACTACCACAAGCTGCTGGATGCTAAAGAGAAGGAAAGACTGGCT | |
| | GCCAGACAGAACTGGACAACAGTTGAAAACATCAAGGAACTGAAGGAAGGCTACC | |
| | TGTCCGTGATCATCCACAACATCTGCGAGCTGGTGAAAAAGTACAACGCTGTGAT | |
| | CGCTATGGAGGACCTGAGCAGCGGCTTCAAGAACAGCCGCGTGAAGGTGGAAAA | |
| | GCAGGTATACCAGAAGTTCGAAAAAATGCTGACCGAGAAACTGAACTTCCTGGTG | |
| | GACAAGAAAGCCGATGTGCAAAGCAGAGGCGGCCTGCTGCAGGCCTACCAGCTG | |
| | ACAAATAGCACAAAGGATTACAAGCGGGCCGGCAGCCAAGACGGCATCGTGTTCT | |
| | ACGTGCCTGCCTGGCTGACAAGCAAAATTGACCCTGTGACCGGCTTTGTGGACCT | |
| | GCTGAAACCTAAATACACCAGCGTTCAGGAGGCCAAAGAGCTGTTCAGCAACTTC | |
| | GAGGCCGTCGAGTACATCCCCGAGGAGGACCTGTTCAGCTTCACCTTCGACTACA | |
| | GCAAGTTCCCCAGATGCAGCGTGGCCTACAGAAACAAGTGGACCGTGTACAGTAA | |
| | CGGAGAGAGAATCTACACATTCAGAGATAAGAACAGCAACAACGAATACGTGTCC | |
| | AAGACAGTTGCCCTGACCACCGAGTTTAAAAGCCTCTTCGACGAATATAGCGTGTA | |
| | CTACCGAGACAACCTGAAGAGTCAGATTTTGTGCCAGGATAAGGTGGATTTCTTCA | |
| | AGCAACTTATCAGACTGCTGTCCCTGACCATGCAGATGAGAAACAGCATCAGCAA | |
| | CAGCGCCGTGGACTACCTGATCTCCCCTGTGAAGGATAAGAATGGCAATTTTTTC | |
| | GACAGCAGAAAGAGCATCAAGAACCTGCCTGAGAACGCCGACGCCAACGGCGCC | |
| | TACAACATTGCTAAGAAGGCTCTGTGGGCCATCGGTCAGATCAAAGAGGCTGATG | |
| | AGAATGACCTGATGAAGGTGAAGCTGTCCGTGTCTAATAAAGAGTGGCTGAAGTA | |
| | CGTGCAGGAGGTGGAA | |
| Expression | ATGggctccggaGAATTTGATAACTCTTTCGTGAATAGATATCCTCTGAGCAAGACCCT | 102 |
| construct (with | GAGCTTCAGTCTGCTGCCAGTGGGCAGCACCGAAGCCAACTTCGAAAAAAAGCTG | |
| N-terminal | CTGCTGCAGGAGGACGAAAAGAGAGCCGCCGAGTACATCCTGGTGAAAAGCTAC | |
| methionine | ATCGACAGATACCACAAGGCCTACATCGAGAGCGTGCTGAGCAAGGTGGTGCTG | |
| and stop | GACGGCATCAACAACTATGCCCAGCTGTACTGCAAGAACAACAAGACCGAACAGG | |
| codon, | ACATCAAGCGGCTGGAGCAGCTGGAGGGCAGCTTCAGAAAGCAGATCTCTAAAAG | |
| includes V5- | CCTGAAGTCCGACGCCAGATACAAGCTGATCTACAAAAAGGAGATGCTGGAAAAG | |
| tag and C- | CTCCTGCCTGAGTTCCTGGACAACGAGGAAGAAAAGGCTAGAGTGATCAGCTTCG | |
| terminal NLS) | AGAACTTTACAACCTACTTCACTGGCTTCCACACCAACCGGGAAAACATGTACACC | |
| | GATGAGGCCAAGTCTACGGCCGTTTCCTTTAGGTGTATCAACGATAACCTGCCAAA | |
| | GTTCCTGGACAACATCAGCGTATTCAAGTGGGTCACCGCCTTTCTGAGCGAGTCT | |
| | GACATCAACGAACTGAAGGCCGATTTCAGCGGCCTGTTGGGCTGCTCCCTGGAAG | |
| | AGATGTTCACCCCTGATTACTTCAGCTTCGTGCTGTCTCAGAGCGGCATCGAGAG | |
| | ATACAACAACGTGATCGGCGGATACACCTGTAGCGATGGCGAGAAAGTCAAAGGA | |
| | CTTAATGAGTACATCAACCTGTATAACCAGAAGCTGCAACACGGCGAAAAGAAACT | |
| | GCCCCTGCTCAAGCGGCTGTTCAAGCAGATTCTGTCAGACACCGAGAGCGTGTCC | |
| | TTCATCCCCGAGAAACTGGAAAATGATGACGCCGTGATCTCCGCCATTAACGGATT | |
| | TTGTAATATCAAGATCGAGAATGAAACATTCTTCGAGATCCTGGACAAGACCAAGT | |
| | GCCTGTTCAGCAATCTGAACGAGTTCGACTCTGCCGGAGTGTACATCACCAACGG | |
| | CTTCGCAGTGACAGACATCAGCAACGCCGTGTTCGGCACCTGGGACGTCATCAGC | |
| | GAAGCCTGGAAGAAAGAGTACGCCAAAGCTATCCCCCTGAAGAACATCGCTAAGG | |
| | CCGACGCCTACTATGAGAAGCAGGGCAAGGCCTACAAGGCCATCAAGAGCTTCTC | |
| | TGTAAGCGAACTGCAGAGACTGGCCAACACCACGGAGGGAAAGGCCGCCTACAA | |
| | GCACAACGGCGACATCAGCGCCTATTTCAGCGAGACAGTCTGCTTCGCTGTGCAG | |
| | GATATCTTCGAGAAGTATAGCAGCAGCAAGGCCCTGTTCGCCAGCCCCTATAAGA | |
| | ACGAGAAGCGGCTGTTCAAGAACAATGAGGCAATCGCTCTGATTAAGGACTTCCT | |
| | GGATAGCATCAAGAACCTGGAGAAGCTGATTAAGCCATTCAACGGCAGCGGCAGA | |
| | GAGAACGACAAGGACGAGAGCTTTTACGGCGAGTTCACCGCCTGCTACGAGCGG | |
| | CTGAGCAAAATCGATCTGCTGTACGACAAGGTGCGGAACTACATGACACAGAAAC | |
| | CTTACAGCGGCGATAAGATCAAGCTGAACTTCGAGAATCCTCAGTTCCTGAACGG | |
| | ATGGGATAGAAACAAGGAGCGGGATTACAGAACAGTGCTGCTGAGAAAGGGAGG | |
| | TTATTACTACCTGGCCATCATGGACAAGAGCAACAACCGGATCTTCGAGGATCTGC | |
| | CTGAGCCTAAGAATGGTGAGGACTGCTACGAAAAAATCGATTACAAGCTGCTGCC | |
| | TGGCCCTAACAAGATGCTGCCCAAAGTGTTCTTCGCCGCTAGTAACATCGACTACT | |
| | TCGCCCCTAGCGAACAGATCCTCAAAATCCGGCAGAAGGAAACCTTCAAAAAGGG | |
| | CGTGAACTTCAACATTGACGACTGTCACGCCTTCATCGACTTCCTGAAGGAATCTA | |
| | TCGAGAAGCACGACGAGTGGTGCAAGTACGGCTTCGAGTTTAAGGACACCAGCG | |
| | ACTACAACAATATAGGCGAGTTCTACAAGGACGTGCGGGAACAGGGCTACAGCAT | |
| | CTCTTTTCGGAATGTGCCCGAGTCCTACATCAACAGCTGCGTGAACTCTGGCTCTC | |
| | TGTACCTGTTTCAGATCTACAACAAAGATTTTAGCCCTTACTCTAAGGGCACAAAG | |
| | AGCCTGCACACCCTGTACTTTGAAATGCTGTTTGACGAGCGCAACCTGAAGAACG | |
| | TGGTGTATCAGCTGAATGGTGGCGCTGAGATGTTCTACAGAAAGGCCAGCATCAA | |
| | GGAAAGAGACAAGATCGTGCACCCCGCCAACATCCCTATCAAGAACAAGAACCCC | |
| | GACAACCCTAAGGCCGAGAGCGTGTTCGAATACGACATTATCAAGGACAGAAGAT | |
| | TCACCGAACGGCAGTTCTCCCTGCACATCCCTGTGACCCTGAACTTCAAAGGCTC | |
| | TGGCGGATCTGCCAACCTGAACGCCGACGTTAGGCGGGCTATCAGAGGCGCCGA | |
| | TGAGAACTACGTGATCGGCATCGACCGGGGCGAGAGGAACCTGCTGTACATCAC | |
| | AGTGATCAATAGCAAGGGCGAGATCGTGGAACAAATCCCAGGCAACGTGATCATC | |
| | AACGGCAAGCAAGTGGTGGACTACCACAAGCTGCTGGATGCTAAAGAGAAGGAAA | |
| | GACTGGCTGCCAGACAGAACTGGACAACAGTTGAAAACATCAAGGAACTGAAGGA | |
| | AGGCTACCTGTCCGTGATCATCCACAACATCTGCGAGCTGGTGAAAAAGTACAAC | |
| | GCTGTGATCGCTATGGAGGACCTGAGCAGCGGCTTCAAGAACAGCCGCGTGAAG | |
| | GTGGAAAAGCAGGTATACCAGAAGTTCGAAAAAATGCTGACCGAGAAACTGAACT | |
| | TCCTGGTGGACAAGAAAGCCGATGTGCAAAGCAGAGGCGGCCTGCTGCAGGCCT | |
| | ACCAGCTGACAAATAGCACAAAGGATTACAAGCGGGCCGGCAGCCAAGACGGCA | |
| | TCGTGTTCTACGTGCCTGCCTGGCTGACAAGCAAAATTGACCCTGTGACCGGCTT | |
| | TGTGGACCTGCTGAAACCTAAATACACCAGCGTTCAGGAGGCCAAAGAGCTGTTC | |
| | AGCAACTTCGAGGCCGTCGAGTACATCCCCGAGGAGGACCTGTTCAGCTTCACCT | |
| | TCGACTACAGCAAGTTCCCCAGATGCAGCGTGGCCTACAGAAACAAGTGGACCGT | |
| | GTACAGTAACGGAGAGAGAATCTACACATTCAGAGATAAGAACAGCAACAACGAAT | |
| | ACGTGTCCAAGACAGTTGCCCTGACCACCGAGTTTAAAAGCCTCTTCGACGAATAT | |
| | AGCGTGTACTACCGAGACAACCTGAAGAGTCAGATTTTGTGCCAGGATAAGGTGG | |
| | ATTTCTTCAAGCAACTTATCAGACTGCTGTCCCTGACCATGCAGATGAGAAACAGC | |
| | ATCAGCAACAGCGCCGTGGACTACCTGATCTCCCCTGTGAAGGATAAGAATGGCA | |
| | ATTTTTTCGACAGCAGAAAGAGCATCAAGAACCTGCCTGAGAACGCCGACGCCAA | |
| | CGGCGCCTACAACATTGCTAAGAAGGCTCTGTGGGCCATCGGTCAGATCAAAGAG | |
| | GCTGATGAGAATGACCTGATGAAGGTGAAGCTGTCCGTGTCTAATAAAGAGTGGC | |
| | TGAAGTACGTGCAGGAGGTGGAAtctagaAAGCGGACAGCAGACGGCTCCGAATTT | |
| | GAAAGCCCTAAGAAAAAGAGAAAGGTGggatccGGCAAACCTATCCCCAATCCCCTG | |
| | CTGGGCCTGGACAGCACCTGA | |

In some embodiments a ZRBH Type V Cas protein comprises an amino acid sequence of SEQ ID NO: 97, SEQ ID NO:98, or SEQ ID NO:99. In some embodiments, a ZRBH Type V Cas protein has nickase activity, for example resulting from one or more amino acid substitutions relative to the sequence of SEQ ID NO:97, SEQ ID NO:98, or SEQ ID NO:99. In some embodiments, the one or more amino acid substitutions providing nickase activity comprise a D851 substitution, wherein the position of the D851 substitution is defined with respect to the amino acid numbering of SEQ ID NO:98 (corresponding to amino acid 908 of SEQ ID NO: 121). In some embodiments, the one or more amino acid substitutions providing nickase activity comprise an E940 substitution, wherein the position of the E940 substitution is defined with respect to the amino acid numbering of SEQ ID NO:98 (corresponding to amino acid 993 of SEQ ID NO: 121). In some embodiments, the one or more amino acid substitutions providing nickase activity comprise a R1152 substitution, wherein the position of the R1152 substitution is defined with respect to the amino acid numbering of SEQ ID NO:98 (corresponding to amino acid 1226 of SEQ ID NO: 121). In some embodiments, the one or more amino acid substitutions providing nickase activity comprise a D1189 substitution, wherein the position of the D1189 substitution is defined with respect to the amino acid numbering of SEQ ID NO:98 (corresponding to amino acid 1263 of SEQ ID NO: 121). In some embodiments, a ZRBH Type V Cas protein is catalytically inactive, for example due to a R1152 substitution in combination with a D851 substitution, a E940 substitution, and/or D1152 substitution.

### 6.2.18. ZWPU Type V Cas Protein

In one aspect, the disclosure provides ZWPU Type V Cas proteins. ZWPU Type V Cas proteins can be further classified as Type V-A Cas proteins. The ZWPU Type V Cas proteins typically comprise an amino acid sequence that is at least 50%, at least 55%, at least 60%, at least 65%, at least 70%, at least 75%, at least 85%, at least 90%, or at least 95% identical to SEQ ID NO: 103. In some embodiments, the ZWPU Type V Cas proteins comprise an amino acid sequence that is at least 95%, at least 96%, at least 97%, at least 98%, or at least 99% identical to SEQ ID NO: 103. In some embodiments, a ZWPU Type V Cas protein comprises an amino acid sequence that is identical to SEQ ID NO: 103.

Exemplary ZWPU Type V Cas protein sequences and nucleotide sequences encoding exemplary ZWPU Type V Cas proteins are set forth in Table 1R.

TABLE 1R
ZWPU Type V Cas Sequences

| | | SEQ |
|---|---|---|
| | | ID |
| Name | Sequence | NO. |
| Wildtype | KAETNLTELVNLYSLQKTLRFELIPQGKTLENIEKNGILTQDNQRADDYEKVKKLIDEY | 103 |
| amino acid | HKHHIEISLDDCRLEGLEEYKELYEKKDDLKKIQENLRKQIVKSLTENERYKDKRLFSD | |
| sequence | KLFKEDLPNYLKDREQDKALVKKFEKFTTYFTGFNENRKNMYSSEDKPTSIAYRLIHE | |
| (without N- | NLPKFIDNLHIFDKIKETTIKDDFDKIVEKLNKHLKIHIKSFDEIFSIEYFNKTLSQKQIDNY | |
| terminal | NNIIGGMSFENGTKIQGLNEYINRYNQKQEDKHQKLPCVKTLYKQILSDREKISWIPEQ | |
| methionine) | FDDDKQMAESISNLYNEMLPIIKDDLLPLMANIGDYDLSKIFISNDSALTTISQRIFGAYN | |
| | VYTLAIIEKLKSDKPKSKRQSESKYLDEIDKNFKNMKSFSIAKLNNAVKGKYDKTIENYI | |
| | KVFGAFDEEENLLQRLETAYNEAEPILNNIEDRCKNINQDKDAVEKIKTLLDALKDIQH | |
| | FAKLLLCDNDETEIDAEFYNKLHDIVVKLDKITPIYNMVRNYVTKKPYSEEKIKLNFEKS | |
| | TLLGGWDLNKEKNNLSVILRKDNLYYLGIMKKDNNKIFDSTNIKTDGVCFEKMEYKLL | |
| | PDPKKMLPKVFFSKKCSKDFNPNDKILEIKENESFKKTSSNFNIEQCRKLIDFYKESIN | |
| | KHKDWQKFNFQFSDTKTYNDINEFYNEVEKQGYKISFCKISEDYINELVKDNKLYLFKI | |
| | WNKDFSKYSKGTPNTHTLYWKQIFAPENINNVVYKLNGQAEIFFRQASISQKNVIKHL | |
| | ANKPVKNKNIKNEKKESTFSYDLVKDKRFTMDKFHFHVPITINFKAKGINNTNPIVNNLI | |
| | RQNKIEHIIGIDRGERHLLYLSLIDLKGNIIEQKSLNEIINNYNGNEYKTDYHTLLDDKEK | |
| | ERKDARLSWNTIENIKELKDGYMSQWVHIISQMIVKYNAIVVLEDLNHGFVRGRQKIEK | |
| | QVYEKFEHKLIDKLNYYVDKNADSNAVGGLYNALQLTNPFDSFEKLGKQSGCLFYIPA | |
| | WKTSKIDPVTGFINMFTNLKYESVEKSKKFFSKFDDIRYNKEKNRFEFDVSFDKFDSD | |
| | FVRITQESKLHWTLCSVGQRIELVKENNGYKPNEINLTDAFKSVFNTNKIEINTAKLNR | |
| | EIGKINDTAFFKELMRLMKLLLQMRNSKPNSIEKNDDYIISPVADENGVFFDSSKVEDN | |
| | GNLPKDADANGAYNIARKGLYVIHQIKQSEDDKKIDFKDFNPRWLKFIQQKLYLND | |
| Wildtype | MKAETNLTELVNLYSLQKTLRFELIPQGKTLENIEKNGILTQDNQRADDYEKVKKLIDE | 104 |
| amino acid | YHKHHIEISLDDCRLEGLEEYKELYEKKDDLKKIQENLRKQIVKSLTENERYKDKRLFS | |
| sequence (with | DKLFKEDLPNYLKDREQDKALVKKFEKFTTYFTGFNENRKNMYSSEDKPTSIAYRLIH | |
| N-terminal | ENLPKFIDNLHIFDKIKETTIKDDFDKIVEKLNKHLKIHIKSFDEIFSIEYFNKTLSQKQIDN | |
| methionine) | YNNIIGGMSFENGTKIQGLNEYINRYNQKQEDKHQKLPCVKTLYKQILSDREKISWIPE | |
| | QFDDDKQMAESISNLYNEMLPIIKDDLLPLMANIGDYDLSKIFISNDSALTTISQRIFGAY | |
| | NVYTLAIIEKLKSDKPKSKRQSESKYLDEIDKNFKNMKSFSIAKLNNAVKGKYDKTIEN | |
| | YIKVFGAFDEEENLLQRLETAYNEAEPILNNIEDRCKNINQDKDAVEKIKTLLDALKDIQ | |
| | HFAKLLLCDNDETEIDAEFYNKLHDIWVKLDKITPIYNMVRNYVTKKPYSEEKIKLNFEK | |
| | STLLGGWDLNKEKNNLSVILRKDNLYYLGIMKKDNNKIFDSTNIKTDGVCFEKMEYKL | |
| | LPDPKKMLPKVFFSKKCSKDFNPNDKILEIKENESFKKTSSNFNIEQCRKLIDFYKESIN | |
| | KHKDWQKFNFQFSDTKTYNDINEFYNEVEKQGYKISFCKISEDYINELVKDNKLYLFKI | |
| | WNKDFSKYSKGTPNTHTLYWKQIFAPENINNVVYKLNGQAEIFFRQASISQKNVIKHL | |
| | ANKPVKNKNIKNEKKESTFSYDLVKDKRFTMDKFHFHVPITINFKAKGINNTNPIVNNLI | |
| | RQNKIEHIIGIDRGERHLLYLSLIDLKGNIIEQKSLNEIINNYNGNEYKTDYHTLLDDKEK | |
| | ERKDARLSWNTIENIKELKDGYMSQWVHIISQMIVKYNAIVVLEDLNHGFVRGRQKIEK | |
| | QVYEKFEHKLIDKLNYYVDKNADSNAVGGLYNALQLTNPFDSFEKLGKQSGCLFYIPA | |
| | WKTSKIDPVTGFINMFTNLKYESVEKSKKFFSKFDDIRYNKEKNRFEFDVSFDKFDSD | |
| | FVRITQESKLHWTLCSVGQRIELVKENNGYKPNEINLTDAFKSVFNTNKIEINTAKLNR | |
| | EIGKINDTAFFKELMRLMKLLLQMRNSKPNSIEKNDDYIISPVADENGVFFDSSKVEDN | |
| | GNLPKDADANGAYNIARKGLYVIHQIKQSEDDKKIDFKDFNPRWLKFIQQKLYLND | |
| Expression | MGSGKAETNLTELVNLYSLQKTLRFELIPQGKTLENIEKNGILTQDNQRADDYEKVKK | 105 |
| construct (with | LIDEYHKHHIEISLDDCRLEGLEEYKELYEKKDDLKKIQENLRKQIVKSLTENERYKDK | |
| N-terminal | RLFSDKLFKEDLPNYLKDREQDKALVKKFEKFTTYFTGFNENRKNMYSSEDKPTSIAY | |
| methionine, | RLIHENLPKFIDNLHIFDKIKETTIKDDFDKIVEKLNKHLKIHIKSFDEIFSIEYFNKTLSQK | |
| V5-tag and C- | QIDNYNNIIGGMSFENGTKIQGLNEYINRYNQKQEDKHQKLPCVKTLYKQILSDREKIS | |
| terminal NLS) | WIPEQFDDDKQMAESISNLYNEMLPIIKDDLLPLMANIGDYDLSKIFISNDSALTTISQRI | |
| aa sequence | FGAYNVYTLAIIEKLKSDKPKSKRQSESKYLDEIDKNFKNMKSFSIAKLNNAVKGKYDK | |
| | TIENYIKVFGAFDEEENLLQRLETAYNEAEPILNNIEDRCKNINQDKDAVEKIKTLLDAL | |
| | KDIQHFAKLLLCDNDETEIDAEFYNKLHDIWVKLDKITPIYNMVRNYVTKKPYSEEKIKL | |
| | NFEKSTLLGGWDLNKEKNNLSVILRKDNLYYLGIMKKDNNKIFDSTNIKTDGVCFEKM | |
| | EYKLLPDPKKMLPKVFFSKKCSKDFNPNDKILEIKENESFKKTSSNFNIEQCRKLIDFY | |
| | KESINKHKDWQKFNFQFSDTKTYNDINEFYNEVEKQGYKISFCKISEDYINELVKDNKL | |
| | YLFKIWNKDFSKYSKGTPNTHTLYWKQIFAPENINNVVYKLNGQAEIFFRQASISQKN | |
| | VIKHLANKPVKNKNIKNEKKESTFSYDLVKDKRFTMDKFHFHVPITINFKAKGINNTNPI | |
| | VNNLIRQNKIEHIIGIDRGERHLLYLSLIDLKGNIIEQKSLNEIINNYNGNEYKTDYHTLLD | |
| | DKEKERKDARLSWNTIENIKELKDGYMSQVVHIISQMIVKYNAIVVLEDLNHGFVRGR | |
| | QKIEKQVYEKFEHKLIDKLNYYVDKNADSNAVGGLYNALQLTNPFDSFEKLGKQSGC | |
| | LFYIPAWKTSKIDPVTGFINMFTNLKYESVEKSKKFFSKFDDIRYNKEKNRFEFDVSFD | |
| | KFDSDFVRITQESKLHWTLCSVGQRIELVKENNGYKPNEINLTDAFKSVENTNKIEINT | |
| | AKLNREIGKINDTAFFKELMRLMKLLLQMRNSKPNSIEKNDDYIISPVADENGVFFDSS | |
| | KVEDNGNLPKDADANGAYNIARKGLYVIHQIKQSEDDKKIDFKDFNPRWLKFIQQKLY | |
| | LNDSRKRTADGSEFESPKKKRKVGSGKPIPNPLLGLDST | |
| Wildtype | ATGAAAGCAGAAACAAATCTGACAGAATTAGTGAATCTGTATTCATTGCAGAAAAC | 106 |
| coding | ACTTCGTTTTGAATTAATCCCACAGGGCAAAACATTAGAAAACATTGAGAAAAATG | |
| sequence (with | GTATTCTTACACAAGATAACCAAAGAGCAGACGATTACGAAAAAGTCAAAAAACTT | |
| N-terminal | ATTGATGAGTATCATAAGCACCATATTGAAATAAGTCTTGACGATTGTCGCCTTGA | |
| methionine | AGGTTTAGAGGAATATAAAGAACTCTACGAAAAGAAAGATGATTTGAAAAAAATTC | |
| and stop | AAGAGAATCTACGAAAACAAATCGTTAAAAGTTTAACGGAGAACGAAAGGTATAA | |
| codon) | AGACAAACGTCTATTCTCTGATAAACTCTTCAAAGAAGATCTTCCGAATTATCTAA | |
| | AAGATAGAGAACAAGACAAAGCTCTTGTTAAAAAATTTGAAAAATTCACCACATAT | |
| | TTTACTGGATTTAACGAAAACAGAAAAAATATGTATTCTTCCGAAGACAAACCTAC | |
| | CTCAATTGCTTATAGATTAATCCATGAAAATTTACCTAAGTTTATAGACAATTTACA | |
| | TATTTTTGATAAAATTAAAGAAACAACAATCAAAGATGATTTTGATAAGATTGTTGA | |
| | AAAATTAAACAAGCATCTAAAAATTCATATCAAATCATTTGACGAAATTTTCTCTAT | |
| | TGAATATTTCAATAAAACTCTTAGCCAAAAACAAATAGACAATTATAACAATATAAT | |
| | TGGAGGAATGTCTTTTGAGAATGGTACAAAGATACAAGGCTTAAACGAATATATTA | |
| | ATCGTTACAATCAAAAGCAGGAAGATAAACATCAAAAACTTCCTTGCGTCAAAACA | |
| | CTTTATAAGCAAATACTCAGTGATAGAGAAAAAATATCGTGGATTCCAGAACAATT | |
| | TGATGATGATAAACAAATGGCAGAAAGTATTTCGAATTTGTACAATGAAATGCTTC | |
| | CAATTATTAAAGATGATCTACTTCCGCTAATGGCTAATATAGGCGATTATGATCTT | |
| | AGCAAAATATTTATCTCCAACGACTCTGCTTTAACAACAATATCTCAACGAATTTTT | |
| | GGAGCTTACAACGTTTACACTCTTGCAATAATAGAAAAATTAAAAAGTGATAAACC | |
| | TAAATCAAAAAGACAATCCGAGTCTAAGTATTTAGACGAAATTGACAAAAACTTCA | |
| | AAAATATGAAAAGTTTCAGTATTGCAAAACTAAACAATGCCGTAAAAGGCAAATAC | |
| | GATAAAACAATAGAAAATTATATCAAGGTTTTCGGGGCTTTTGACGAAGAAGAGAA | |
| | CTTGCTACAACGATTAGAAACAGCCTATAACGAAGCTGAGCCTATACTTAATAATA | |
| | TAGAAGACAGATGCAAAAATATTAATCAAGACAAAGATGCTGTTGAAAAGATTAAA | |
| | ACATTATTAGATGCTTTGAAAGATATTCAACATTTTGCAAAACTTCTATTATGTGAT | |
| | AACGACGAAACTGAAATAGATGCGGAGTTTTATAATAAATTACATGATATATGGGT | |
| | AAAATTGGACAAGATAACACCTATATATAATATGGTGAGAAATTATGTTACAAAGA | |
| | AACCTTATTCAGAAGAAAAAATCAAATTGAATTTTGAAAAATCTACACTATTAGGC | |
| | GGCTGGGATTTGAACAAAGAAAAAAATAATTTATCAGTTATACTCCGCAAAGATAA | |
| | TTTGTATTACTTAGGGATTATGAAAAAAGATAATAACAAAATCTTTGATAGTACAAA | |
| | TATCAAAACCGATGGCGTTTGTTTTGAGAAAATGGAATACAAACTACTTCCTGATC | |
| | CAAAGAAAATGCTGCCAAAGGTATTCTTTTCAAAAAAATGTTCAAAGGACTTTAAC | |
| | CCGAACGACAAAATATTAGAAATTAAGGAAAATGAAAGTTTCAAGAAAACAAGCA | |
| | GTAATTTCAATATTGAGCAATGTCGTAAATTAATAGACTTCTATAAAGAATCTATCA | |
| | ATAAACATAAAGATTGGCAAAAATTTAATTTCCAATTCTCTGACACTAAAACTTACA | |
| | ATGACATAAACGAATTTTACAACGAAGTTGAAAAACAAGGTTATAAAATATCTTTTT | |
| | GTAAAATTTCTGAGGATTATATAAATGAGTTGGTGAAAGACAATAAACTTTATTTGT | |
| | TTAAGATTTGGAACAAAGACTTTTCAAAATATAGCAAAGGAACTCCAAATACGCAC | |
| | ACTCTTTATTGGAAACAAATATTTGCACCTGAAAATATCAACAATGTCGTATATAAA | |
| | CTAAACGGACAAGCCGAAATATTTTTTAGGCAAGCAAGTATTTCTCAAAAAAACGT | |
| | TATCAAACATTTGGCAAACAAACCTGTTAAAAACAAGAATATAAAAAACGAAAAAA | |
| | AGGAAAGTACGTTCAGTTATGATTTAGTAAAAGATAAACGTTTTACTATGGATAAA | |
| | TTCCATTTCCACGTACCGATTACTATTAATTTCAAGGCAAAAGGAATAAATAATAC | |
| | CAATCCTATTGTCAATAATCTAATTCGTCAAAACAAGATAGAACATATTATTGGTAT | |
| | AGATAGAGGCGAAAGGCATTTGCTTTATCTTTCTCTTATAGATTTGAAAGGAAATA | |
| | TCATTGAACAAAAGTCGTTGAATGAAATCATAAACAACTACAATGGCAATGAATAT | |
| | AAAACAGATTACCATACCTTGCTTGATGATAAGGAAAAAGAAAGAAAAGATGCCC | |
| | GACTTTCGTGGAATACTATTGAAAATATCAAAGAACTCAAAGACGGGTATATGAG | |
| | CCAAGTTGTGCATATTATCTCACAAATGATTGTGAAGTACAATGCAATAGTTGTTT | |
| | TGGAAGACCTTAATCATGGCTTTGTTCGTGGTCGCCAGAAGATAGAAAAACAAGT | |
| | TTATGAAAAATTTGAGCATAAACTTATTGATAAACTAAACTATTATGTCGATAAGAA | |
| | TGCCGATAGCAATGCCGTTGGAGGACTTTACAATGCTTTGCAACTAACAAATCCA | |
| | TTTGATAGTTTTGAAAAATTAGGAAAACAAAGCGGCTGTTTATTCTATATCCCTGC | |
| | TTGGAAAACAAGTAAGATTGATCCCGTTACTGGATTTATTAATATGTTTACAAATCT | |
| | CAAATACGAATCAGTGGAAAAATCAAAGAAGTTCTTTTCAAAGTTTGACGATATTA | |
| | GATACAATAAAGAAAAAAATAGGTTTGAATTTGATGTTTCATTTGATAAATTCGATA | |
| | GTGATTTTGTCCGTATTACACAGGAAAGTAAATTACATTGGACGCTTTGCAGTGTT | |
| | GGTCAGCGTATAGAATTAGTAAAAGAGAATAATGGTTATAAACCTAATGAAATAAA | |
| | TTTAACTGATGCTTTCAAATCAGTGTTTAATACTAATAAAATAGAGATAAACACTGC | |
| | TAAACTGAATAGAGAGATTGGTAAAATCAATGATACAGCGTTTTTCAAGGAACTTA | |
| | TGCGTTTAATGAAATTGTTATTACAAATGAGAAATAGTAAGCCAAATTCAATAGAG | |
| | AAGAACGACGATTATATTATCTCTCCTGTTGCAGACGAAAATGGAGTATTCTTTGA | |
| | CAGCAGTAAAGTTGAAGACAATGGCAATTTGCCAAAAGATGCCGATGCCAACGG | |
| | AGCATACAATATTGCTCGCAAAGGCTTGTATGTAATACACCAAATAAAGCAAAGC | |
| | GAAGATGATAAAAAAATCGATTTCAAAGATTTCAACCCACGTTGGTTAAAATTCAT | |
| | TCAGCAAAAACTATATTTGAATGATTGA | |
| Codon | AAGGCCGAGACAAACCTGACAGAACTCGTGAACCTGTACAGCCTGCAAAAAACC | 107 |
| optimized | CTGAGATTTGAGCTCATCCCCCAGGGCAAGACCCTTGAGAACATCGAGAAGAAC | |
| coding | GGTATCCTGACCCAGGACAATCAGAGAGCCGACGACTACGAGAAGGTGAAAAAA | |
| sequence (no | CTGATCGACGAGTACCACAAGCACCACATCGAGATCAGCCTGGACGATTGCAGA | |
| N-terminal | CTGGAAGGCCTGGAAGAATACAAGGAACTGTATGAGAAGAAGGATGACCTAAAG | |
| methionine, no | AAAATCCAGGAAAACCTGAGAAAGCAGATCGTGAAGTCCCTCACTGAGAACGAA | |
| stop codon) | CGGTACAAGGACAAAAGACTCTTCTCAGATAAGCTGTTCAAGGAAGATCTGCCTA | |
| | ATTACCTGAAGGACAGAGAACAGGACAAGGCCCTGGTAAAAAAGTTCGAGAAGT | |
| | TCACCACCTACTTCACCGGCTTCAACGAAAACCGCAAAAACATGTACAGCAGCGA | |
| | GGATAAGCCCACCAGCATCGCTTATAGACTGATCCACGAGAACCTGCCTAAGTTC | |
| | ATCGACAACCTGCACATCTTTGATAAGATCAAGGAAACCACCATCAAGGACGATT | |
| | TCGATAAGATCGTGGAAAAGCTGAATAAACACCTGAAGATCCACATCAAATCCTT | |
| | CGACGAGATCTTTTCTATTGAATACTTCAACAAGACACTGAGTCAAAAGCAAATCG | |
| | ACAACTACAACAACATCATCGGCGGAATGAGCTTCGAGAATGGCACCAAGATCCA | |
| | GGGCCTGAATGAGTACATCAACAGATACAACCAGAAACAAGAGGACAAGCATCA | |
| | AAAGCTGCCTTGCGTGAAAACCCTGTACAAGCAGATCCTGAGCGACAGAGAGAA | |
| | GATTTCCTGGATTCCTGAACAGTTCGATGACGACAAACAGATGGCCGAGAGCATC | |
| | AGCAATCTGTACAACGAGATGCTGCCAATCATCAAGGACGACCTGCTGCCTCTGA | |
| | TGGCCAACATTGGCGACTACGACCTGAGCAAAATCTTCATCAGCAATGACAGCG | |
| | CCCTGACAACCATCTCGCAGCGGATCTTCGGAGCTTACAACGTGTACACCCTGG | |
| | CCATCATTGAGAAGCTGAAGTCTGATAAGCCTAAGAGCAAGCGGCAGTCTGAGT | |
| | CTAAGTACCTGGACGAGATCGACAAGAACTTCAAGAACATGAAGTCTTTTAGCAT | |
| | CGCCAAGCTGAACAACGCCGTGAAGGGCAAGTATGACAAGACAATCGAAAATTA | |
| | CATCAAGGTGTTTGGCGCCTTTGATGAGGAGGAGAATCTCCTGCAGAGGCTGGA | |
| | AACAGCCTATAACGAGGCCGAGCCTATCCTGAACAACATCGAGGACAGATGCAA | |
| | AAACATCAATCAAGACAAGGATGCCGTGGAAAAGATCAAGACCTTACTGGACGCT | |
| | CTGAAAGATATCCAGCACTTTGCCAAGTTACTGCTGTGCGACAATGACGAAACCG | |
| | AGATTGACGCCGAGTTCTACAACAAGCTGCACGACATCTGGGTGAAGCTGGACA | |
| | AAATCACACCAATCTACAACATGGTGCGGAACTACGTGACCAAGAAGCCCTACTC | |
| | TGAAGAGAAAATCAAGCTGAACTTCGAAAAGTCTACACTGCTGGGCGGCTGGGA | |
| | TCTGAACAAGGAAAAGAACAATCTGAGCGTGATCCTGAGAAAGGACAACCTGTAC | |
| | TACCTGGGCATCATGAAGAAAGACAACAACAAGATCTTCGACTCCACAAACATCA | |
| | AGACCGACGGCGTTTGTTTCGAGAAGATGGAATATAAGCTGTTACCTGACCCTAA | |
| | AAAGATGCTGCCCAAGGTGTTCTTCTCAAAGAAATGCAGCAAGGATTTCAATCCT | |
| | AACGACAAGATCCTGGAGATCAAAGAGAACGAATCTTTCAAGAAAACCTCTAGCA | |
| | ACTTTAATATCGAGCAGTGCAGAAAACTGATCGACTTTTACAAGGAGTCCATCAAT | |
| | AAGCACAAAGACTGGCAGAAATTCAACTTTCAGTTCAGCGATACCAAGACCTACA | |
| | ACGATATCAACGAGTTCTACAACGAGGTGGAAAAACAGGGCTACAAAATTAGCTT | |
| | CTGCAAGATCAGCGAGGACTACATCAATGAGCTGGTTAAGGACAACAAACTGTAC | |
| | CTGTTTAAGATCTGGAACAAGGATTTCAGTAAGTACAGCAAGGGGACCCCTAACA | |
| | CCCACACCCTGTACTGGAAGCAGATCTTCGCCCCTGAGAACATCAACAACGTCG | |
| | TGTACAAGCTGAACGGACAGGCCGAGATCTTCTTCAGACAAGCATCTATCTCCCA | |
| | GAAGAACGTCATCAAGCACCTAGCTAATAAGCCAGTGAAAAACAAGAACATCAAG | |
| | AACGAGAAGAAGGAGAGCACCTTCAGCTACGATCTTGTTAAGGACAAGCGGTTTA | |
| | CAATGGACAAGTTCCACTTCCACGTGCCAATCACCATAAACTTTAAGGCCAAGGG | |
| | CATCAACAACACCAATCCTATTGTCAACAACCTGATCCGGCAGAACAAGATTGAA | |
| | CACATCATCGGCATCGACAGAGGCGAGAGACACCTGCTGTATCTGAGCCTGATC | |
| | GATCTGAAGGGCAACATCATAGAACAGAAGAGCCTGAACGAGATCATCAACAATT | |
| | ACAACGGCAATGAGTACAAGACCGATTACCATACCTTGCTGGATGACAAGGAAAA | |
| | GGAGAGAAAGGATGCTAGACTGAGCTGGAACACCATCGAAAATATCAAGGAACT | |
| | GAAAGATGGCTACATGAGCCAGGTGGTGCACATCATCAGTCAGATGATCGTGAA | |
| | ATACAACGCCATTGTGGTCCTGGAGGATCTCAACCACGGCTTCGTGCGGGGCAG | |
| | ACAGAAGATCGAGAAGCAGGTGTATGAAAAATTTGAACACAAGCTGATCGACAAG | |
| | CTGAATTACTACGTGGACAAGAATGCTGACAGCAACGCCGTGGGAGGACTGTAC | |
| | AATGCCCTGCAGCTGACAAACCCCTTCGACAGCTTCGAGAAGCTGGGCAAGCAG | |
| | AGCGGCTGTCTGTTTTACATCCCCGCCTGGAAAACAAGTAAGATCGATCCTGTGA | |
| | CCGGATTCATCAACATGTTCACCAACCTGAAGTACGAATCTGTGGAAAAGAGCAA | |
| | AAAGTTCTTCAGCAAGTTCGATGACATCAGATACAACAAGGAGAAAAACCGATTC | |
| | GAGTTCGACGTGTCCTTCGACAAGTTCGACTCCGACTTCGTGCGGATCACCCAG | |
| | GAGAGCAAACTGCATTGGACCTTGTGTAGCGTGGGCCAGAGAATCGAACTGGTC | |
| | AAGGAAAACAACGGATACAAGCCTAACGAAATCAACCTGACAGATGCTTTCAAGA | |
| | GCGTGTTCAACACAAACAAGATCGAGATCAACACCGCCAAACTGAATCGGGAAAT | |
| | CGGAAAAATCAACGACACAGCTTTCTTCAAGGAACTGATGCGGCTGATGAAGCTG | |
| | CTCCTGCAGATGAGAAACAGCAAGCCCAACTCCATCGAAAAGAACGATGATTACA | |
| | TCATCAGCCCTGTGGCCGATGAGAACGGCGTGTTCTTTGACAGCAGCAAAGTGG | |
| | AGGACAATGGCAACCTGCCAAAGGACGCCGATGCCAACGGCGCCTACAACATCG | |
| | CCAGGAAGGGCCTGTATGTGATCCACCAGATTAAGCAGTCTGAGGACGACAAGA | |
| | AGATCGACTTTAAGGACTTCAACCCCAGATGGCTGAAGTTCATCCAGCAGAAGCT | |
| | GTACCTGAACGAT | |
| Expression | ATGggctccggaAAGGCCGAGACAAACCTGACAGAACTCGTGAACCTGTACAGCCTG | 108 |
| construct (with | CAAAAAACCCTGAGATTTGAGCTCATCCCCCAGGGCAAGACCCTTGAGAACATC | |
| N-terminal | GAGAAGAACGGTATCCTGACCCAGGACAATCAGAGAGCCGACGACTACGAGAAG | |
| methionine | GTGAAAAAACTGATCGACGAGTACCACAAGCACCACATCGAGATCAGCCTGGAC | |
| and stop | GATTGCAGACTGGAAGGCCTGGAAGAATACAAGGAACTGTATGAGAAGAAGGAT | |
| codon, | GACCTAAAGAAAATCCAGGAAAACCTGAGAAAGCAGATCGTGAAGTCCCTCACTG | |
| includes V5- | AGAACGAACGGTACAAGGACAAAAGACTCTTCTCAGATAAGCTGTTCAAGGAAGA | |
| tag and C- | TCTGCCTAATTACCTGAAGGACAGAGAACAGGACAAGGCCCTGGTAAAAAAGTTC | |
| terminal NLS) | GAGAAGTTCACCACCTACTTCACCGGCTTCAACGAAAACCGCAAAAACATGTACA | |
| | GCAGCGAGGATAAGCCCACCAGCATCGCTTATAGACTGATCCACGAGAACCTGC | |
| | CTAAGTTCATCGACAACCTGCACATCTTTGATAAGATCAAGGAAACCACCATCAA | |
| | GGACGATTTCGATAAGATCGTGGAAAAGCTGAATAAACACCTGAAGATCCACATC | |
| | AAATCCTTCGACGAGATCTTTTCTATTGAATACTTCAACAAGACACTGAGTCAAAA | |
| | GCAAATCGACAACTACAACAACATCATCGGCGGAATGAGCTTCGAGAATGGCAC | |
| | CAAGATCCAGGGCCTGAATGAGTACATCAACAGATACAACCAGAAACAAGAGGA | |
| | CAAGCATCAAAAGCTGCCTTGCGTGAAAACCCTGTACAAGCAGATCCTGAGCGA | |
| | CAGAGAGAAGATTTCCTGGATTCCTGAACAGTTCGATGACGACAAACAGATGGCC | |
| | GAGAGCATCAGCAATCTGTACAACGAGATGCTGCCAATCATCAAGGACGACCTG | |
| | CTGCCTCTGATGGCCAACATTGGCGACTACGACCTGAGCAAAATCTTCATCAGCA | |
| | ATGACAGCGCCCTGACAACCATCTCGCAGCGGATCTTCGGAGCTTACAACGTGT | |
| | ACACCCTGGCCATCATTGAGAAGCTGAAGTCTGATAAGCCTAAGAGCAAGCGGC | |
| | AGTCTGAGTCTAAGTACCTGGACGAGATCGACAAGAACTTCAAGAACATGAAGTC | |
| | TTTTAGCATCGCCAAGCTGAACAACGCCGTGAAGGGCAAGTATGACAAGACAATC | |
| | GAAAATTACATCAAGGTGTTTGGCGCCTTTGATGAGGAGGAGAATCTCCTGCAGA | |
| | GGCTGGAAACAGCCTATAACGAGGCCGAGCCTATCCTGAACAACATCGAGGACA | |
| | GATGCAAAAACATCAATCAAGACAAGGATGCCGTGGAAAAGATCAAGACCTTACT | |
| | GGACGCTCTGAAAGATATCCAGCACTTTGCCAAGTTACTGCTGTGCGACAATGAC | |
| | GAAACCGAGATTGACGCCGAGTTCTACAACAAGCTGCACGACATCTGGGTGAAG | |
| | CTGGACAAAATCACACCAATCTACAACATGGTGCGGAACTACGTGACCAAGAAGC | |
| | CCTACTCTGAAGAGAAAATCAAGCTGAACTTCGAAAAGTCTACACTGCTGGGCGG | |
| | CTGGGATCTGAACAAGGAAAAGAACAATCTGAGCGTGATCCTGAGAAAGGACAA | |
| | CCTGTACTACCTGGGCATCATGAAGAAAGACAACAACAAGATCTTCGACTCCACA | |
| | AACATCAAGACCGACGGCGTTTGTTTCGAGAAGATGGAATATAAGCTGTTACCTG | |
| | ACCCTAAAAAGATGCTGCCCAAGGTGTTCTTCTCAAAGAAATGCAGCAAGGATTT | |
| | CAATCCTAACGACAAGATCCTGGAGATCAAAGAGAACGAATCTTTCAAGAAAACC | |
| | TCTAGCAACTTTAATATCGAGCAGTGCAGAAAACTGATCGACTTTTACAAGGAGT | |
| | CCATCAATAAGCACAAAGACTGGCAGAAATTCAACTTTCAGTTCAGCGATACCAA | |
| | GACCTACAACGATATCAACGAGTTCTACAACGAGGTGGAAAAACAGGGCTACAAA | |
| | ATTAGCTTCTGCAAGATCAGCGAGGACTACATCAATGAGCTGGTTAAGGACAACA | |
| | AACTGTACCTGTTTAAGATCTGGAACAAGGATTTCAGTAAGTACAGCAAGGGGAC | |
| | CCCTAACACCCACACCCTGTACTGGAAGCAGATCTTCGCCCCTGAGAACATCAAC | |
| | AACGTCGTGTACAAGCTGAACGGACAGGCCGAGATCTTCTTCAGACAAGCATCTA | |
| | TCTCCCAGAAGAACGTCATCAAGCACCTAGCTAATAAGCCAGTGAAAAACAAGAA | |
| | CATCAAGAACGAGAAGAAGGAGAGCACCTTCAGCTACGATCTTGTTAAGGACAA | |
| | GCGGTTTACAATGGACAAGTTCCACTTCCACGTGCCAATCACCATAAACTTTAAG | |
| | GCCAAGGGCATCAACAACACCAATCCTATTGTCAACAACCTGATCCGGCAGAACA | |
| | AGATTGAACACATCATCGGCATCGACAGAGGCGAGAGACACCTGCTGTATCTGA | |
| | GCCTGATCGATCTGAAGGGCAACATCATAGAACAGAAGAGCCTGAACGAGATCA | |
| | TCAACAATTACAACGGCAATGAGTACAAGACCGATTACCATACCTTGCTGGATGA | |
| | CAAGGAAAAGGAGAGAAAGGATGCTAGACTGAGCTGGAACACCATCGAAAATAT | |
| | CAAGGAACTGAAAGATGGCTACATGAGCCAGGTGGTGCACATCATCAGTCAGAT | |
| | GATCGTGAAATACAACGCCATTGTGGTCCTGGAGGATCTCAACCACGGCTTCGT | |
| | GCGGGGCAGACAGAAGATCGAGAAGCAGGTGTATGAAAAATTTGAACACAAGCT | |
| | GATCGACAAGCTGAATTACTACGTGGACAAGAATGCTGACAGCAACGCCGTGGG | |
| | AGGACTGTACAATGCCCTGCAGCTGACAAACCCCTTCGACAGCTTCGAGAAGCT | |
| | GGGCAAGCAGAGCGGCTGTCTGTTTTACATCCCCGCCTGGAAAACAAGTAAGAT | |
| | CGATCCTGTGACCGGATTCATCAACATGTTCACCAACCTGAAGTACGAATCTGTG | |
| | GAAAAGAGCAAAAAGTTCTTCAGCAAGTTCGATGACATCAGATACAACAAGGAGA | |
| | AAAACCGATTCGAGTTCGACGTGTCCTTCGACAAGTTCGACTCCGACTTCGTGCG | |
| | GATCACCCAGGAGAGCAAACTGCATTGGACCTTGTGTAGCGTGGGCCAGAGAAT | |
| | CGAACTGGTCAAGGAAAACAACGGATACAAGCCTAACGAAATCAACCTGACAGAT | |
| | GCTTTCAAGAGCGTGTTCAACACAAACAAGATCGAGATCAACACCGCCAAACTGA | |
| | ATCGGGAAATCGGAAAAATCAACGACACAGCTTTCTTCAAGGAACTGATGCGGCT | |
| | GATGAAGCTGCTCCTGCAGATGAGAAACAGCAAGCCCAACTCCATCGAAAAGAA | |
| | CGATGATTACATCATCAGCCCTGTGGCCGATGAGAACGGCGTGTTCTTTGACAG | |
| | CAGCAAAGTGGAGGACAATGGCAACCTGCCAAAGGACGCCGATGCCAACGGCG | |
| | CCTACAACATCGCCAGGAAGGGCCTGTATGTGATCCACCAGATTAAGCAGTCTG | |
| | AGGACGACAAGAAGATCGACTTTAAGGACTTCAACCCCAGATGGCTGAAGTTCAT | |
| | CCAGCAGAAGCTGTACCTGAACGATtctagaAAGCGGACAGCAGACGGCTCCGAAT | |
| | TTGAAAGCCCTAAGAAAAAGAGAAAGGTGggatccGGCAAACCTATCCCCAATCCC | |
| | CTGCTGGGCCTGGACAGCACCTGA | |

In some embodiments a ZWPU Type V Cas protein comprises an amino acid sequence of SEQ ID NO: 103, SEQ ID NO: 104, or SEQ ID NO: 105. In some embodiments, a ZWPU Type V Cas protein has nickase activity, for example resulting from one or more amino acid substitutions relative to the sequence of SEQ ID NO: 103, SEQ ID NO: 104, or SEQ ID NO: 105. In some embodiments, the one or more amino acid substitutions providing nickase activity comprise a D845 substitution, wherein the position of the D845 substitution is defined with respect to the amino acid numbering of SEQ ID NO: 104 (corresponding to amino acid 908 of SEQ ID NO: 121). In some embodiments, the one or more amino acid substitutions providing nickase activity comprise an E938 substitution, wherein the position of the E938 substitution is defined with respect to the amino acid numbering of SEQ ID NO: 104 (corresponding to amino acid 993 of SEQ ID NO: 121). In some embodiments, the one or more amino acid substitutions providing nickase activity comprise a R1153 substitution, wherein the position of the R1153 substitution is defined with respect to the amino acid numbering of SEQ ID NO: 104 (corresponding to amino acid 1226 of SEQ ID NO: 121). In some embodiments, the one or more amino acid substitutions providing nickase activity comprise a D1195 substitution, wherein the position of the D1195 substitution is defined with respect to the amino acid numbering of SEQ ID NO: 104 (corresponding to amino acid 1263 of SEQ ID NO: 121). In some embodiments, a ZWPU Type V Cas protein is catalytically inactive, for example due to a R1153 substitution in combination with a D845 substitution, a E938 substitution, and/or D1195 substitution.

### 6.2.19. ZZQE Type V Cas Protein

In one aspect, the disclosure provides ZZQE Type V Cas proteins. ZZQE Type V Cas proteins can be further classified as Type V-A Cas proteins. The ZZQE Type V Cas proteins typically comprise an amino acid sequence that is at least 50%, at least 55%, at least 60%, at least 65%, at least 70%, at least 75%, at least 85%, at least 90%, or at least 95% identical to SEQ ID NO: 109. In some embodiments, the ZZQE Type V Cas proteins comprise an amino acid sequence that is at least 95%, at least 96%, at least 97%, at least 98%, or at least 99% identical to SEQ ID NO: 109. In some embodiments, a ZZQE Type V Cas protein comprises an amino acid sequence that is identical to SEQ ID NO: 109.

Exemplary ZZQE Type V Cas protein sequences and nucleotide sequences encoding exemplary ZZQE Type V Cas proteins are set forth in Table 1S.

TABLE 1S
ZZQE Type V Cas Sequences

| | | SEQ |
|---|---|---|
| | | ID |
| Name | Sequence | NO. |
| Wildtype | DMKSLNSFQNQYSLSKTLRFQLIPQGKTLDNINESRILEEDQHRSESYKLVKKIIDDYH | 109 |
| amino acid | KAYIEQALGSFELKIASDSKNDSLEEFYSQYIAERKEDKAKKLFEKTQDNLRKQISKKL | |
| sequence | KQGEAYKRLFGKELIQEDLLEFVATDPEADSKKRLIEEFKDFTTYFIGFHENRKNMYA | |
| (without N- | EEAQSTAIAYRIIHENLPKFIDNIRTFEELAKSSIADVLPQVYEDFKAYLKVESVKELFSL | |
| terminal | DYFNTVLTQKQLDIYNAVIGGKSLDENSRIQGLNEYINLYNQQHKDKKLPFLKPLFKQI | |
| methionine) | LSDRNSLSWLPEAFDNDKQVLQAVHDCYTSLLESVFHKDGLQQLLQSLPTYNLKGIY | |
| | LRNDLSMTNVSQKLLGDWGAITRAVKEKLQKENPAKKRESDEAYQERINKIFKQAGS | |
| | YSLDYINQALEATDQTNIKVEDYFINMGVDNEQKEPLFQRVAQAYNQASDLLEKEYPA | |
| | NKNLMQDKESIEHIKFLLDNLKAVQHFIKPLLGDGNEADKDNRFYGELTALWNELDQV | |
| | TRLYNKVRNYMTRKPYSVDKIKINFKNSTLLNGWDRNKERDNTAVILRKDGKFYLAIM | |
| | HKEHNKVFEKFPVGTKDSDFEKMEYKLLPGANKMLPKVFFSKSRIDEFKPSAELLQK | |
| | YQMGTHKKGELFSLNDCHSLIDFFKASIEKHDDWKQFNFHFSPTSSYEDLSGFYREV | |
| | EQQGYKLTFKSVDADYINKMVDEGKIFLFQIYNKDFSEHSKGTPNLHTLYWKMLFDE | |
| | RNLQNVVYKLNGEAEVFFRKKSLTYTRPTHPKKEPIKNKNVQNAKKESIFDYDLIKNK | |
| | RFTVDSFQFHVPITMNFKSEGRSNLNERVNEFLRQNNDAHIIGIDRGERHLLYLVVIDR | |
| | HGNIVEQFSLNSIINEYQGNTYATNYHDLLDKREKEREEARESWQSIENIKELKEGYL | |
| | SQVVHKIADLMVKYHAIVVLEDLNMGFMRGRQKVEKQVYQKFEKMLIDKLNYLVDKK | |
| | QDAETDGGLLKAYQLTNQFESFQKLGKQSGFLFYVPAWNTSKIDPCTGFTNLLDTRY | |
| | ESIEKAKKFFQTFNAIRYNAAQGYFEFELDYNKFNKRADGTQTLWTLCTYGPRIETLR | |
| | STEDNNKWTSKEVDLTDELKKHFYHYGIKLDADLKEAIGQQTDKPFFTNLLHLLKLTL | |
| | QMRNSKIGTEVDYLISPIRNEDGTFYDSRQGNKSLPANADANGAYNIARKGLWVINQI | |
| | KQTPQDQKPKLAITNKEWLQFAQEKPYLKD | |
| Wildtype | MDMKSLNSFQNQYSLSKTLRFQLIPQGKTLDNINESRILEEDQHRSESYKLVKKIIDDY | 110 |
| amino acid | HKAYIEQALGSFELKIASDSKNDSLEEFYSQYIAERKEDKAKKLFEKTQDNLRKQISKK | |
| sequence (with | LKQGEAYKRLFGKELIQEDLLEFVATDPEADSKKRLIEEFKDFTTYFIGFHENRKNMYA | |
| N-terminal | EEAQSTAIAYRIIHENLPKFIDNIRTFEELAKSSIADVLPQVYEDFKAYLKVESVKELFSL | |
| methionine) | DYFNTVLTQKQLDIYNAVIGGKSLDENSRIQGLNEYINLYNQQHKDKKLPFLKPLFKQI | |
| | LSDRNSLSWLPEAFDNDKQVLQAVHDCYTSLLESVFHKDGLQQLLQSLPTYNLKGIY | |
| | LRNDLSMTNVSQKLLGDWGAITRAVKEKLQKENPAKKRESDEAYQERINKIFKQAGS | |
| | YSLDYINQALEATDQTNIKVEDYFINMGVDNEQKEPLFQRVAQAYNQASDLLEKEYPA | |
| | NKNLMQDKESIEHIKFLLDNLKAVQHFIKPLLGDGNEADKDNRFYGELTALWNELDQV | |
| | TRLYNKVRNYMTRKPYSVDKIKINFKNSTLLNGWDRNKERDNTAVILRKDGKFYLAIM | |
| | HKEHNKVFEKFPVGTKDSDFEKMEYKLLPGANKMLPKVFFSKSRIDEFKPSAELLQK | |
| | YQMGTHKKGELFSLNDCHSLIDFFKASIEKHDDWKQFNFHFSPTSSYEDLSGFYREV | |
| | EQQGYKLTFKSVDADYINKMVDEGKIFLFQIYNKDFSEHSKGTPNLHTLYWKMLFDE | |
| | RNLQNVVYKLNGEAEVFFRKKSLTYTRPTHPKKEPIKNKNVQNAKKESIFDYDLIKNK | |
| | RFTVDSFQFHVPITMNFKSEGRSNLNERVNEFLRQNNDAHIIGIDRGERHLLYLVVIDR | |
| | HGNIVEQFSLNSIINEYQGNTYATNYHDLLDKREKEREEARESWQSIENIKELKEGYL | |
| | SQVVHKIADLMVKYHAIVVLEDLNMGFMRGRQKVEKQVYQKFEKMLIDKLNYLVDKK | |
| | QDAETDGGLLKAYQLTNQFESFQKLGKQSGFLFYVPAWNTSKIDPCTGFTNLLDTRY | |
| | ESIEKAKKFFQTFNAIRYNAAQGYFEFELDYNKFNKRADGTQTLWTLCTYGPRIETLR | |
| | STEDNNKWTSKEVDLTDELKKHFYHYGIKLDADLKEAIGQQTDKPFFTNLLHLLKLTL | |
| | QMRNSKIGTEVDYLISPIRNEDGTFYDSRQGNKSLPANADANGAYNIARKGLWVINQI | |
| | KQTPQDQKPKLAITNKEWLQFAQEKPYLKD | |
| Expression | MGSGDMKSLNSFQNQYSLSKTLRFQLIPQGKTLDNINESRILEEDQHRSESYKLVKKII | 111 |
| construct (with | DDYHKAYIEQALGSFELKIASDSKNDSLEEFYSQYIAERKEDKAKKLFEKTQDNLRKQI | |
| N-terminal | SKKLKQGEAYKRLFGKELIQEDLLEFVATDPEADSKKRLIEEFKDFTTYFIGFHENRKN | |
| methionine, | MYAEEAQSTAIAYRIIHENLPKFIDNIRTFEELAKSSIADVLPQVYEDFKAYLKVESVKE | |
| V5-tag and C- | LFSLDYFNTVLTQKQLDIYNAVIGGKSLDENSRIQGLNEYINLYNQQHKDKKLPFLKPL | |
| terminal NLS) | FKQILSDRNSLSWLPEAFDNDKQVLQAVHDCYTSLLESVFHKDGLQQLLQSLPTYNL | |
| aa sequence | KGIYLRNDLSMTNVSQKLLGDWGAITRAVKEKLQKENPAKKRESDEAYQERINKIFKQ | |
| | AGSYSLDYINQALEATDQTNIKVEDYFINMGVDNEQKEPLFQRVAQAYNQASDLLEK | |
| | EYPANKNLMQDKESIEHIKFLLDNLKAVQHFIKPLLGDGNEADKDNRFYGELTALWNE | |
| | LDQVTRLYNKVRNYMTRKPYSVDKIKINFKNSTLLNGWDRNKERDNTAVILRKDGKF | |
| | YLAIMHKEHNKVFEKFPVGTKDSDFEKMEYKLLPGANKMLPKVFFSKSRIDEFKPSAE | |
| | LLQKYQMGTHKKGELFSLNDCHSLIDFFKASIEKHDDWKQFNFHFSPTSSYEDLSGF | |
| | YREVEQQGYKLTFKSVDADYINKMVDEGKIFLFQIYNKDFSEHSKGTPNLHTLYWKM | |
| | LFDERNLQNVVYKLNGEAEVFFRKKSLTYTRPTHPKKEPIKNKNVQNAKKESIFDYDLI | |
| | KNKRFTVDSFQFHVPITMNFKSEGRSNLNERVNEFLRQNNDAHIIGIDRGERHLLYLV | |
| | VIDRHGNIVEQFSLNSIINEYQGNTYATNYHDLLDKREKEREEARESWQSIENIKELKE | |
| | GYLSQVVHKIADLMVKYHAIVVLEDLNMGFMRGRQKVEKQVYQKFEKMLIDKLNYLV | |
| | DKKQDAETDGGLLKAYQLTNQFESFQKLGKQSGFLFYVPAWNTSKIDPCTGFTNLLD | |
| | TRYESIEKAKKFFQTFNAIRYNAAQGYFEFELDYNKFNKRADGTQTLWTLCTYGPRIE | |
| | TLRSTEDNNKWTSKEVDLTDELKKHFYHYGIKLDADLKEAIGQQTDKPFFTNLLHLLK | |
| | LTLQMRNSKIGTEVDYLISPIRNEDGTFYDSRQGNKSLPANADANGAYNIARKGLWVI | |
| | NQIKQTPQDQKPKLAITNKEWLQFAQEKPYLKDSRKRTADGSEFESPKKKRKVGSG | |
| | KPIPNPLLGLDST | |
| Wildtype | ATGGATATGAAAAGTTTAAACAGCTTTCAGAACCAGTATTCCCTATCCAAGACCCT | 112 |
| coding | CCGGTTTCAGCTAATACCCCAGGGTAAAACTTTGGATAACATTAACGAGAGCAGA | |
| sequence (with | ATATTGGAGGAAGACCAACACCGAAGCGAAAGCTACAAGTTGGTCAAGAAAATCA | |
| N-terminal | TTGACGACTATCACAAGGCCTACATCGAACAAGCCCTGGGCAGTTTCGAACTCAA | |
| methionine | AATTGCCAGTGACTCTAAAAACGATTCGTTAGAGGAGTTCTACTCGCAGTATATTG | |
| and stop | CCGAACGGAAAGAAGATAAAGCCAAAAAACTTTTCGAAAAGACGCAAGACAACTT | |
| codon) | GCGAAAGCAAATCTCCAAGAAATTAAAGCAGGGCGAAGCCTACAAGCGGTTGTTT | |
| | GGCAAGGAACTCATTCAAGAAGACCTGCTGGAGTTTGTAGCTACCGACCCTGAG | |
| | GCTGATAGCAAAAAGCGTCTGATTGAAGAATTCAAGGACTTCACCACCTACTTTAT | |
| | CGGATTCCACGAGAACCGAAAGAACATGTATGCTGAGGAAGCCCAATCCACAGC | |
| | AATTGCCTACCGCATCATTCACGAGAACCTGCCGAAGTTCATTGATAACATACGC | |
| | ACCTTCGAAGAACTTGCTAAAAGTTCCATTGCCGACGTCCTGCCACAGGTTTATG | |
| | AAGATTTCAAAGCGTACTTAAAGGTCGAATCGGTCAAAGAACTTTTCAGTCTGGA | |
| | CTATTTCAATACCGTCTTGACCCAAAAGCAGCTTGACATTTACAATGCGGTTATCG | |
| | GCGGTAAGTCGTTAGATGAGAACAGCCGCATCCAGGGGCTCAACGAGTATATCA | |
| | ACCTGTACAACCAGCAGCACAAGGACAAAAAGTTACCCTTCTTAAAACCCTTGTT | |
| | CAAGCAAATTCTGAGCGACCGCAACAGCCTTTCGTGGTTGCCCGAAGCTTTCGA | |
| | CAATGACAAGCAGGTACTTCAGGCTGTACACGACTGCTACACCTCGCTATTGGAG | |
| | AGCGTATTCCACAAAGACGGCCTGCAACAGTTGCTACAGTCACTGCCTACCTACA | |
| | ACCTGAAGGGCATTTACCTGCGCAACGACCTTTCCATGACCAACGTTTCTCAAAA | |
| | ACTATTGGGCGATTGGGGAGCTATTACACGTGCCGTTAAAGAAAAACTACAAAAA | |
| | GAAAATCCTGCCAAAAAACGAGAGTCGGACGAAGCCTACCAAGAACGCATCAAC | |
| | AAGATATTCAAGCAAGCCGGCAGCTACTCTTTAGATTACATCAACCAAGCGCTCG | |
| | AAGCAACAGACCAGACCAATATCAAAGTCGAAGACTACTTCATCAACATGGGCGT | |
| | AGACAACGAGCAAAAAGAGCCCCTGTTCCAGCGTGTAGCGCAAGCCTACAATCA | |
| | GGCCAGCGATTTGCTTGAAAAGGAATATCCCGCAAACAAAAATCTGATGCAGGAT | |
| | AAAGAAAGCATCGAGCACATCAAATTCTTGCTCGATAACCTCAAAGCCGTTCAAC | |
| | ACTTTATAAAGCCCCTGCTCGGCGATGGTAACGAGGCTGATAAAGATAATCGTTT | |
| | TTACGGAGAACTTACAGCGCTGTGGAACGAATTAGACCAGGTAACGCGCCTGTA | |
| | TAACAAGGTGCGAAACTACATGACCCGCAAGCCCTACTCGGTTGATAAAATCAAG | |
| | ATTAACTTTAAGAACTCAACTCTACTTAATGGCTGGGACAGAAATAAGGAACGTGA | |
| | CAATACCGCTGTTATTCTGCGCAAAGACGGCAAGTTCTATCTGGCCATTATGCAT | |
| | AAAGAACACAATAAGGTGTTCGAAAAATTCCCGGTCGGAACAAAGGATTCTGACT | |
| | TCGAGAAAATGGAGTATAAGTTACTTCCGGGCGCCAATAAAATGCTTCCGAAGGT | |
| | TTTCTTCTCTAAATCGCGTATCGATGAGTTTAAGCCCAGCGCCGAACTTCTCCAAA | |
| | AGTACCAGATGGGTACCCACAAAAAGGGCGAACTCTTCAGTCTGAACGACTGCC | |
| | ATTCTCTGATTGACTTCTTTAAGGCTTCTATTGAAAAGCATGACGATTGGAAACAG | |
| | TTTAACTTCCATTTCTCACCCACTTCGAGCTACGAAGACTTGAGCGGATTTTACAG | |
| | AGAGGTTGAACAGCAGGGGTACAAACTGACCTTCAAATCCGTTGACGCCGACTA | |
| | TATCAACAAAATGGTTGACGAGGGCAAAATCTTTCTCTTCCAGATTTACAATAAAG | |
| | ACTTCTCGGAACATAGCAAAGGCACCCCCAACCTGCATACGCTCTACTGGAAAAT | |
| | GCTCTTTGACGAACGCAACCTGCAGAACGTGGTCTACAAACTGAACGGCGAGGC | |
| | CGAAGTCTTCTTCCGGAAGAAGAGTCTTACCTACACCCGTCCTACGCACCCCAAG | |
| | AAAGAGCCTATCAAGAACAAGAACGTTCAGAATGCCAAAAAGGAAAGCATCTTCG | |
| | ACTACGACCTGATTAAAAACAAACGCTTTACGGTCGACTCCTTCCAGTTCCACGT | |
| | TCCCATCACGATGAACTTCAAGAGCGAAGGACGCTCCAACCTGAACGAGCGGGT | |
| | CAACGAGTTTTTACGCCAGAACAACGATGCCCACATCATTGGCATTGACCGGGG | |
| | CGAACGCCATTTGCTCTACCTGGTGGTTATTGACCGGCACGGAAACATTGTGGAA | |
| | CAATTTTCGCTCAACTCTATCATCAACGAATATCAGGGTAATACGTACGCCACCAA | |
| | CTACCACGACTTGTTGGATAAGCGCGAAAAGGAAAGAGAGGAAGCACGCGAAAG | |
| | CTGGCAGAGTATTGAGAATATTAAAGAACTGAAAGAAGGATACTTGAGCCAGGTG | |
| | GTGCATAAAATTGCCGACCTCATGGTAAAGTATCATGCCATCGTGGTGCTCGAAG | |
| | ACTTGAATATGGGCTTCATGCGCGGACGCCAGAAGGTAGAAAAGCAGGTCTATC | |
| | AGAAGTTTGAAAAAATGCTGATAGACAAGTTAAACTATCTGGTTGACAAGAAGCAA | |
| | GATGCCGAAACCGACGGCGGTCTGCTCAAGGCATACCAACTGACCAACCAGTTC | |
| | GAAAGTTTCCAGAAGTTAGGCAAGCAGAGCGGTTTCCTCTTCTATGTGCCTGCCT | |
| | GGAACACCAGCAAAATTGACCCCTGCACCGGATTTACCAACCTGCTCGACACTC | |
| | GATACGAGAGCATCGAAAAGGCCAAAAAGTTCTTTCAAACTTTCAATGCCATCCG | |
| | CTACAATGCTGCGCAGGGGTACTTTGAGTTCGAACTGGATTACAATAAATTCAAC | |
| | AAGCGGGCCGATGGTACACAAACCCTATGGACGCTCTGCACCTACGGCCCACGC | |
| | ATCGAAACACTCCGAAGCACCGAGGATAATAACAAGTGGACAAGCAAAGAGGTT | |
| | GATTTGACCGACGAATTGAAAAAGCACTTCTACCACTATGGCATTAAGCTGGATG | |
| | CCGACCTGAAGGAAGCCATCGGCCAACAAACCGACAAACCTTTCTTCACCAACTT | |
| | GCTCCATCTGCTCAAACTAACACTGCAAATGCGAAACAGCAAAATCGGCACGGA | |
| | GGTTGACTACCTCATTTCGCCAATTCGCAATGAAGACGGAACGTTCTACGACAGC | |
| | CGACAAGGCAACAAATCATTGCCTGCCAATGCCGATGCCAATGGTGCCTACAAC | |
| | ATTGCCCGAAAGGGTTTATGGGTAATTAACCAGATAAAACAAACACCTCAAGACC | |
| | AAAAGCCCAAGTTAGCTATTACCAACAAGGAATGGCTGCAATTTGCTCAAGAGAA | |
| | GCCCTACCTTAAGGATTGA | |
| Codon | GACATGAAGAGCCTGAACTCTTTTCAGAACCAATACTCTCTGAGCAAAACCCTGC | 113 |
| optimized | GGTTCCAGCTGATCCCTCAGGGCAAGACACTGGATAATATCAACGAGAGCAGAA | |
| coding | TCCTGGAAGAGGATCAGCACAGAAGCGAGTCATATAAACTGGTGAAGAAGATCAT | |
| sequence (no | TGACGACTATCACAAGGCCTACATCGAGCAGGCCCTGGGCAGCTTCGAGCTGAA | |
| N-terminal | AATTGCCTCCGATAGCAAGAACGACAGCCTGGAGGAGTTCTACTCTCAGTACATT | |
| methionine, no | GCGGAGAGAAAGGAGGACAAGGCCAAGAAGCTGTTCGAAAAGACCCAGGACAA | |
| stop codon) | TCTGAGAAAGCAGATCTCCAAGAAGCTGAAACAGGGTGAAGCCTACAAACGGCT | |
| | GTTCGGCAAAGAACTGATCCAGGAGGACCTGCTGGAGTTCGTGGCCACAGATCC | |
| | TGAGGCCGACTCTAAGAAGAGACTGATCGAAGAGTTCAAGGACTTTACCACCTAC | |
| | TTCATCGGATTTCACGAAAATAGAAAGAACATGTACGCCGAGGAGGCTCAGAGCA | |
| | CAGCTATTGCCTACAGAATCATCCACGAGAACCTGCCAAAGTTTATCGATAATATC | |
| | AGAACCTTCGAGGAACTGGCCAAGAGCAGCATCGCCGACGTGCTGCCCCAGGT | |
| | CTACGAGGACTTTAAGGCCTACCTGAAGGTGGAAAGCGTGAAAGAACTGTTCTCT | |
| | CTGGATTATTTCAACACCGTGCTGACACAGAAACAACTGGACATCTACAATGCCG | |
| | TGATCGGCGGAAAAAGCCTGGACGAGAACAGCAGAATCCAGGGCCTGAACGAG | |
| | TACATCAACCTCTACAACCAGCAGCATAAGGACAAGAAGCTGCCTTTCCTGAAGC | |
| | CCCTGTTCAAGCAAATCCTGTCCGATAGAAACAGCCTGTCCTGGCTGCCTGAGG | |
| | CCTTCGACAACGACAAGCAGGTGCTGCAGGCCGTGCACGACTGCTACACCAGCC | |
| | TGCTGGAATCTGTGTTCCACAAGGACGGCCTGCAACAGCTGCTGCAGAGCCTCC | |
| | CAACCTACAACTTAAAAGGCATCTACCTGCGGAACGACCTTAGCATGACCAATGT | |
| | GTCCCAGAAGCTGCTGGGCGATTGGGGCGCTATCACCAGAGCCGTGAAGGAAA | |
| | AGCTGCAGAAGGAAAACCCTGCCAAGAAGAGAGAGTCGGACGAGGCCTACCAG | |
| | GAGCGGATCAACAAGATCTTCAAGCAGGCCGGCTCATATTCACTGGATTACATCA | |
| | ACCAGGCCCTCGAAGCCACAGACCAGACAAACATCAAAGTGGAGGACTACTTTA | |
| | TCAACATGGGCGTGGATAATGAGCAGAAAGAGCCTCTGTTTCAAAGGGTGGCCC | |
| | AGGCCTATAACCAGGCCAGCGACCTGCTGGAAAAAGAATACCCCGCTAACAAGA | |
| | ATCTGATGCAGGACAAGGAGAGCATCGAGCACATCAAATTCCTGCTCGACAACCT | |
| | TAAGGCCGTGCAGCACTTCATCAAGCCTCTGCTGGGAGATGGCAACGAAGCCGA | |
| | CAAGGACAACAGATTCTACGGCGAGCTAACCGCCCTGTGGAACGAACTTGACCA | |
| | GGTGACCCGCCTGTACAACAAGGTGCGGAATTACATGACCAGGAAGCCTTACAG | |
| | CGTGGACAAGATCAAAATCAACTTCAAGAACAGCACCCTGCTGAACGGATGGGA | |
| | CAGAAACAAGGAACGGGACAACACAGCTGTCATCCTGAGAAAGGACGGCAAGTT | |
| | CTACCTCGCCATCATGCACAAGGAACACAACAAGGTCTTTGAGAAGTTTCCTGTG | |
| | GGCACTAAGGATTCTGACTTCGAGAAGATGGAATACAAGCTGCTGCCCGGCGCC | |
| | AACAAGATGCTGCCTAAGGTTTTCTTTAGCAAGAGCAGAATCGACGAGTTCAAGC | |
| | CATCTGCCGAGCTGCTGCAGAAGTACCAGATGGGAACTCACAAGAAGGGAGAAC | |
| | TGTTCAGCCTGAACGATTGCCACAGCCTGATCGACTTCTTCAAAGCCTCTATCGA | |
| | GAAGCACGATGATTGGAAGCAGTTCAACTTCCATTTCAGCCCTACCAGCAGCTAC | |
| | GAGGACCTGAGCGGCTTCTACCGGGAGGTGGAACAGCAGGGCTACAAGCTGAC | |
| | CTTCAAGAGCGTGGACGCTGATTACATCAATAAGATGGTCGATGAAGGCAAAATC | |
| | TTCCTGTTCCAGATCTACAACAAGGATTTTAGCGAGCACAGCAAGGGCACACCTA | |
| | ACCTGCACACCCTGTACTGGAAGATGCTGTTCGACGAGAGAAACCTGCAGAACG | |
| | TGGTGTACAAGCTGAACGGCGAAGCTGAGGTGTTCTTTCGGAAGAAGAGCCTGA | |
| | CCTACACACGCCCCACCCACCCTAAGAAGGAGCCTATCAAGAACAAAAACGTGC | |
| | AGAACGCTAAAAAGGAAAGCATCTTCGATTACGACCTGATCAAGAACAAAAGATT | |
| | CACAGTGGATTCTTTCCAGTTCCACGTGCCTATCACAATGAACTTCAAATCTGAG | |
| | GGCAGAAGCAACCTGAATGAGAGGGTGAACGAGTTCCTGAGACAAAACAACGAT | |
| | GCCCACATCATCGGAATCGACAGAGGCGAAAGGCATCTGCTGTACCTGGTGGTG | |
| | ATTGATAGACACGGCAACATCGTGGAACAATTTAGCCTGAACAGCATAATCAATG | |
| | AGTACCAAGGCAATACCTACGCCACAAACTATCACGACCTCCTGGACAAGAGAG | |
| | AGAAGGAGCGGGAAGAGGCCAGAGAGTCCTGGCAGTCTATCGAGAACATCAAG | |
| | GAGCTCAAAGAAGGCTACCTGAGTCAGGTGGTGCACAAAATCGCCGACCTGATG | |
| | GTGAAGTATCACGCCATCGTGGTGCTGGAGGACCTGAACATGGGCTTCATGAGA | |
| | GGCCGACAGAAGGTAGAGAAGCAGGTTTACCAGAAATTCGAGAAGATGCTGATT | |
| | GACAAGCTGAACTATCTGGTGGACAAAAAGCAAGATGCTGAAACCGACGGCGGC | |
| | CTGCTCAAGGCCTACCAACTGACCAACCAGTTCGAGAGCTTCCAGAAGCTGGGC | |
| | AAACAGTCTGGCTTCCTGTTTTACGTGCCCGCCTGGAACACCAGCAAGATCGATC | |
| | CCTGTACAGGCTTCACCAACCTGCTGGACACCCGATACGAGAGCATCGAAAAAG | |
| | CAAAGAAGTTCTTCCAAACATTCAACGCCATAAGATACAACGCTGCTCAGGGGTA | |
| | TTTTGAGTTCGAGCTCGACTACAACAAGTTTAACAAGCGGGCCGATGGCACCCA | |
| | GACCCTGTGGACACTGTGCACCTACGGACCTAGAATCGAAACCCTGCGGAGCAC | |
| | AGAGGACAACAACAAGTGGACCAGCAAAGAGGTGGACCTGACAGACGAGCTGAA | |
| | GAAACACTTCTACCACTACGGCATCAAGTTGGATGCCGACCTGAAAGAGGCCAT | |
| | CGGCCAGCAAACAGACAAGCCCTTCTTCACCAACCTGCTGCACCTGCTGAAGCT | |
| | GACACTGCAGATGAGAAACAGCAAGATCGGAACCGAGGTGGACTACCTGATTAG | |
| | CCCCATCAGAAACGAAGATGGCACCTTCTACGACAGCAGACAGGGAAACAAGAG | |
| | CCTGCCTGCTAATGCGGACGCCAATGGCGCCTACAACATCGCTAGAAAAGGCCT | |
| | CTGGGTCATCAACCAGATCAAACAGACCCCTCAGGATCAGAAACCTAAGCTGGC | |
| | CATCACCAATAAGGAGTGGCTGCAGTTCGCCCAGGAGAAACCATACCTGAAAGA | |
| | C | |
| Expression | ATGggctccggaGACATGAAGAGCCTGAACTCTTTTCAGAACCAATACTCTCTGAGCA | 114 |
| construct (with | AAACCCTGCGGTTCCAGCTGATCCCTCAGGGCAAGACACTGGATAATATCAACG | |
| N-terminal | AGAGCAGAATCCTGGAAGAGGATCAGCACAGAAGCGAGTCATATAAACTGGTGA | |
| methionine | AGAAGATCATTGACGACTATCACAAGGCCTACATCGAGCAGGCCCTGGGCAGCT | |
| and stop | TCGAGCTGAAAATTGCCTCCGATAGCAAGAACGACAGCCTGGAGGAGTTCTACT | |
| codon, | CTCAGTACATTGCGGAGAGAAAGGAGGACAAGGCCAAGAAGCTGTTCGAAAAGA | |
| includes V5- | CCCAGGACAATCTGAGAAAGCAGATCTCCAAGAAGCTGAAACAGGGTGAAGCCT | |
| tag and C- | ACAAACGGCTGTTCGGCAAAGAACTGATCCAGGAGGACCTGCTGGAGTTCGTGG | |
| terminal NLS) | CCACAGATCCTGAGGCCGACTCTAAGAAGAGACTGATCGAAGAGTTCAAGGACT | |
| | TTACCACCTACTTCATCGGATTTCACGAAAATAGAAAGAACATGTACGCCGAGGA | |
| | GGCTCAGAGCACAGCTATTGCCTACAGAATCATCCACGAGAACCTGCCAAAGTTT | |
| | ATCGATAATATCAGAACCTTCGAGGAACTGGCCAAGAGCAGCATCGCCGACGTG | |
| | CTGCCCCAGGTCTACGAGGACTTTAAGGCCTACCTGAAGGTGGAAAGCGTGAAA | |
| | GAACTGTTCTCTCTGGATTATTTCAACACCGTGCTGACACAGAAACAACTGGACA | |
| | TCTACAATGCCGTGATCGGCGGAAAAAGCCTGGACGAGAACAGCAGAATCCAGG | |
| | GCCTGAACGAGTACATCAACCTCTACAACCAGCAGCATAAGGACAAGAAGCTGC | |
| | CTTTCCTGAAGCCCCTGTTCAAGCAAATCCTGTCCGATAGAAACAGCCTGTCCTG | |
| | GCTGCCTGAGGCCTTCGACAACGACAAGCAGGTGCTGCAGGCCGTGCACGACT | |
| | GCTACACCAGCCTGCTGGAATCTGTGTTCCACAAGGACGGCCTGCAACAGCTGC | |
| | TGCAGAGCCTCCCAACCTACAACTTAAAAGGCATCTACCTGCGGAACGACCTTAG | |
| | CATGACCAATGTGTCCCAGAAGCTGCTGGGCGATTGGGGCGCTATCACCAGAGC | |
| | CGTGAAGGAAAAGCTGCAGAAGGAAAACCCTGCCAAGAAGAGAGAGTCGGACG | |
| | AGGCCTACCAGGAGCGGATCAACAAGATCTTCAAGCAGGCCGGCTCATATTCAC | |
| | TGGATTACATCAACCAGGCCCTCGAAGCCACAGACCAGACAAACATCAAAGTGG | |
| | AGGACTACTTTATCAACATGGGCGTGGATAATGAGCAGAAAGAGCCTCTGTTTCA | |
| | AAGGGTGGCCCAGGCCTATAACCAGGCCAGCGACCTGCTGGAAAAAGAATACCC | |
| | CGCTAACAAGAATCTGATGCAGGACAAGGAGAGCATCGAGCACATCAAATTCCT | |
| | GCTCGACAACCTTAAGGCCGTGCAGCACTTCATCAAGCCTCTGCTGGGAGATGG | |
| | CAACGAAGCCGACAAGGACAACAGATTCTACGGCGAGCTAACCGCCCTGTGGAA | |
| | CGAACTTGACCAGGTGACCCGCCTGTACAACAAGGTGCGGAATTACATGACCAG | |
| | GAAGCCTTACAGCGTGGACAAGATCAAAATCAACTTCAAGAACAGCACCCTGCTG | |
| | AACGGATGGGACAGAAACAAGGAACGGGACAACACAGCTGTCATCCTGAGAAAG | |
| | GACGGCAAGTTCTACCTCGCCATCATGCACAAGGAACACAACAAGGTCTTTGAGA | |
| | AGTTTCCTGTGGGCACTAAGGATTCTGACTTCGAGAAGATGGAATACAAGCTGCT | |
| | GCCCGGCGCCAACAAGATGCTGCCTAAGGTTTTCTTTAGCAAGAGCAGAATCGA | |
| | CGAGTTCAAGCCATCTGCCGAGCTGCTGCAGAAGTACCAGATGGGAACTCACAA | |
| | GAAGGGAGAACTGTTCAGCCTGAACGATTGCCACAGCCTGATCGACTTCTTCAAA | |
| | GCCTCTATCGAGAAGCACGATGATTGGAAGCAGTTCAACTTCCATTTCAGCCCTA | |
| | CCAGCAGCTACGAGGACCTGAGCGGCTTCTACCGGGAGGTGGAACAGCAGGGC | |
| | TACAAGCTGACCTTCAAGAGCGTGGACGCTGATTACATCAATAAGATGGTCGATG | |
| | AAGGCAAAATCTTCCTGTTCCAGATCTACAACAAGGATTTTAGCGAGCACAGCAA | |
| | GGGCACACCTAACCTGCACACCCTGTACTGGAAGATGCTGTTCGACGAGAGAAA | |
| | CCTGCAGAACGTGGTGTACAAGCTGAACGGCGAAGCTGAGGTGTTCTTTCGGAA | |
| | GAAGAGCCTGACCTACACACGCCCCACCCACCCTAAGAAGGAGCCTATCAAGAA | |
| | CAAAAACGTGCAGAACGCTAAAAAGGAAAGCATCTTCGATTACGACCTGATCAAG | |
| | AACAAAAGATTCACAGTGGATTCTTTCCAGTTCCACGTGCCTATCACAATGAACTT | |
| | CAAATCTGAGGGCAGAAGCAACCTGAATGAGAGGGTGAACGAGTTCCTGAGACA | |
| | AAACAACGATGCCCACATCATCGGAATCGACAGAGGCGAAAGGCATCTGCTGTA | |
| | CCTGGTGGTGATTGATAGACACGGCAACATCGTGGAACAATTTAGCCTGAACAG | |
| | CATAATCAATGAGTACCAAGGCAATACCTACGCCACAAACTATCACGACCTCCTG | |
| | GACAAGAGAGAGAAGGAGCGGGAAGAGGCCAGAGAGTCCTGGCAGTCTATCGA | |
| | GAACATCAAGGAGCTCAAAGAAGGCTACCTGAGTCAGGTGGTGCACAAAATCGC | |
| | CGACCTGATGGTGAAGTATCACGCCATCGTGGTGCTGGAGGACCTGAACATGGG | |
| | CTTCATGAGAGGCCGACAGAAGGTAGAGAAGCAGGTTTACCAGAAATTCGAGAA | |
| | GATGCTGATTGACAAGCTGAACTATCTGGTGGACAAAAAGCAAGATGCTGAAACC | |
| | GACGGCGGCCTGCTCAAGGCCTACCAACTGACCAACCAGTTCGAGAGCTTCCAG | |
| | AAGCTGGGCAAACAGTCTGGCTTCCTGTTTTACGTGCCCGCCTGGAACACCAGC | |
| | AAGATCGATCCCTGTACAGGCTTCACCAACCTGCTGGACACCCGATACGAGAGC | |
| | ATCGAAAAAGCAAAGAAGTTCTTCCAAACATTCAACGCCATAAGATACAACGCTG | |
| | CTCAGGGGTATTTTGAGTTCGAGCTCGACTACAACAAGTTTAACAAGCGGGCCGA | |
| | TGGCACCCAGACCCTGTGGACACTGTGCACCTACGGACCTAGAATCGAAACCCT | |
| | GCGGAGCACAGAGGACAACAACAAGTGGACCAGCAAAGAGGTGGACCTGACAG | |
| | ACGAGCTGAAGAAACACTTCTACCACTACGGCATCAAGTTGGATGCCGACCTGAA | |
| | AGAGGCCATCGGCCAGCAAACAGACAAGCCCTTCTTCACCAACCTGCTGCACCT | |
| | GCTGAAGCTGACACTGCAGATGAGAAACAGCAAGATCGGAACCGAGGTGGACTA | |
| | CCTGATTAGCCCCATCAGAAACGAAGATGGCACCTTCTACGACAGCAGACAGGG | |
| | AAACAAGAGCCTGCCTGCTAATGCGGACGCCAATGGCGCCTACAACATCGCTAG | |
| | AAAAGGCCTCTGGGTCATCAACCAGATCAAACAGACCCCTCAGGATCAGAAACCT | |
| | AAGCTGGCCATCACCAATAAGGAGTGGCTGCAGTTCGCCCAGGAGAAACCATAC | |
| | CTGAAAGACtctagaAAGCGGACAGCAGACGGCTCCGAATTTGAAAGCCCTAAGAA | |
| | AAAGAGAAAGGTGggatccGGCAAACCTATCCCCAATCCCCTGCTGGGCCTGGACA | |
| | GCACCTGA | |

In some embodiments a ZZQE Type V Cas protein comprises an amino acid sequence of SEQ ID NO: 109, SEQ ID NO: 110, or SEQ ID NO: 111. In some embodiments, a ZZQE Type V Cas protein has nickase activity, for example resulting from one or more amino acid substitutions relative to the sequence of SEQ ID NO: 109, SEQ ID NO:110, or SEQ ID NO: 111. In some embodiments, the one or more amino acid substitutions providing nickase activity comprise a D859 substitution, wherein the position of the D859 substitution is defined with respect to the amino acid numbering of SEQ ID NO: 110 (corresponding to amino acid 908 of SEQ ID NO: 121). In some embodiments, the one or more amino acid substitutions providing nickase activity comprise an E952 substitution, wherein the position of the E952 substitution is defined with respect to the amino acid numbering of SEQ ID NO: 110 (corresponding to amino acid 993 of SEQ ID NO: 121). In some embodiments, the one or more amino acid substitutions providing nickase activity comprise a R1164 substitution, wherein the position of the R1164 substitution is defined with respect to the amino acid numbering of SEQ ID NO: 110 (corresponding to amino acid 1226 of SEQ ID NO: 121). In some embodiments, the one or more amino acid substitutions providing nickase activity comprise a D1201 substitution, wherein the position of the D1201 substitution is defined with respect to the amino acid numbering of SEQ ID NO: 110 (corresponding to amino acid 1263 of SEQ ID NO: 121). In some embodiments, a ZZQE Type V Cas protein is catalytically inactive, for example due to a R1164 substitution in combination with a D859 substitution, a E952 substitution, and/or D1201 substitution.

### 6.2.20. ZRXE Type V Cas Protein

In one aspect, the disclosure provides ZRXE Type V Cas proteins. ZRXE Type V Cas proteins can be further classified as Type V-A Cas proteins. The ZRXE Type V Cas proteins typically comprise an amino acid sequence that is at least 50%, at least 55%, at least 60%, at least 65%, at least 70%, at least 75%, at least 85%, at least 90%, or at least 95% identical to SEQ ID NO: 115. In some embodiments, the ZRXE Type V Cas proteins comprise an amino acid sequence that is at least 95%, at least 96%, at least 97%, at least 98%, or at least 99% identical to SEQ ID NO: 115. In some embodiments, a ZRXE Type V Cas protein comprises an amino acid sequence that is identical to SEQ ID NO: 115.

Exemplary ZRXE Type V Cas protein sequences and nucleotide sequences encoding exemplary ZRXE Type V Cas proteins are set forth in Table 1T.

TABLE 1T
ZRXE Type V Cas Sequences

| | | SEQ ID |
|---|---|---|
| Name | Sequence | NO. |
| Wildtype | KAFENFTGLYPLSKTLRFELKPIGKTLEYIEKHGILDKDKHRANSYVKVKDIIDRYHK | 115 |
| amino acid | QFIEDSLSDSDFKLKYENKGKKESLEEYFYYYKLRNRDDKQKKDFDEIQKNLRKQI | |
| sequence | ASQLKKQDRFKRIDKKELIKEDLLEFVSDDNERNLINEFKDFTTYFTGFHENRQNMY | |
| (without N- | SDEAKSTAIAYRLIHENLPKFIDNISVFERVAATDVADCFAQIYSDFEEYLNVNDISEI | |
| terminal | FRLDYYTEILTQTQIDAYNLIIGGRSEGNIKIKGLNEYINLYNQQQKDKSQRLPKLKSL | |
| methionine) | FKQILSDRNAISWLPESFENDNQLLEKLESCYQSFNETYDDKKSIFVRFRELLLTISD | |
| | YEMDKIFLRNDLQLTDISQKMFGSYSIISRSLLEDLKRGTSRKSKKETDESFEERLR | |
| | NIIKNQDSFAIGTIDSSLQQMDVEEYKKSICDYFPNLSVDDKGDDIFDRIVKAYSEVK | |
| | DLLNSPYPSDKNLAQEDDDIDKIKNLLESMKDLQKFVKPLCGKGNESDKDERFYGE | |
| | FTALYEELDKITPLYNMVRNYLTRKPYSTEKIKLNFDNAQLLNGWDLNKESDNTSVI | |
| | LRKDGLYYLAIMNKKHNKVFEKNKLQSDGVCFEKMEYKLLPGANKMLPKVFFSKS | |
| | RIDEFGPSQRLLDSYQNETHKKGDKFNIEDCHELIDFFKRSIDKHEDWSKFSFSFS | |
| | DTKTYEDLSGFYREVEHQGYILSFVNVSVDYVNSLVDEGKIYLFQIYNKDFSPFSKG | |
| | TPNMHTLYWKMLFDEENLKDVVYKLNGQAEVFFRKSSIKYDKPTHPANLPIDNKNV | |
| | SNHKKRSVFEYDLVKDKRYTVDKFQFHVPVTINFKSDGNGNINPLVNDYIKKSDDL | |
| | HVIGIDRGERHLLYLTVIDMKGNIKKQFSLNEIVNEYKGNTYSTNYHDLLEKREDKR | |
| | DKERKEWKTIETIKELKEGYLSQVIHKITELMVEYNAIIVLEDLNLGFMRGRQKVEKS | |
| | VYQKFEKMLIDKLNYLADKKKEPEDLGGVLKAYQLANKFESFQKMGKQSGFLFYT | |
| | QAWNTSKIDPVTGFVNLFDTHYENILKSKNFFSKFDLIKYNSDKDWFEFSFDYNNF | |
| | TTKAEGTKTKWTLCTFGNRIISFRNPDNNMQWDGKEINLTEEFKLFFEKFGININSD | |
| | LHAEILKQDKKDFFEGLLHLLKLTLQMRNSKTRTDIDYMQSPVADENGVLYNSNKC | |
| | GKSLPENADANGAYNIARKGLMIIDKIKKSDNLNKIDLTISNKEWLVFAQNKPYLKN | |
| Wildtype | MKAFENFTGLYPLSKTLRFELKPIGKTLEYIEKHGILDKDKHRANSYVKVKDIIDRYH | 116 |
| amino acid | KQFIEDSLSDSDFKLKYENKGKKESLEEYFYYYKLRNRDDKQKKDFDEIQKNLRKQ | |
| sequence (with | IASQLKKQDRFKRIDKKELIKEDLLEFVSDDNERNLINEFKDFTTYFTGFHENRQNM | |
| N-terminal | YSDEAKSTAIAYRLIHENLPKFIDNISVFERVAATDVADCFAQIYSDFEEYLNVNDISE | |
| methionine) | IFRLDYYTEILTQTQIDAYNLIIGGRSEGNIKIKGLNEYINLYNQQQKDKSQRLPKLKS | |
| | LFKQILSDRNAISWLPESFENDNQLLEKLESCYQSFNETYDDKKSIFVRFRELLLTIS | |
| | DYEMDKIFLRNDLQLTDISQKMFGSYSIISRSLLEDLKRGTSRKSKKETDESFEERL | |
| | RNIIKNQDSFAIGTIDSSLQQMDVEEYKKSICDYFPNLSVDDKGDDIFDRIVKAYSEV | |
| | KDLLNSPYPSDKNLAQEDDDIDKIKNLLESMKDLQKFVKPLCGKGNESDKDERFYG | |
| | EFTALYEELDKITPLYNMVRNYLTRKPYSTEKIKLNFDNAQLLNGWDLNKESDNTS | |
| | VILRKDGLYYLAIMNKKHNKVFEKNKLQSDGVCFEKMEYKLLPGANKMLPKVFFSK | |
| | SRIDEFGPSQRLLDSYQNETHKKGDKFNIEDCHELIDFFKRSIDKHEDWSKFSFSFS | |
| | DTKTYEDLSGFYREVEHQGYILSFVNVSVDYVNSLVDEGKIYLFQIYNKDFSPFSKG | |
| | TPNMHTLYWKMLFDEENLKDVVYKLNGQAEVFFRKSSIKYDKPTHPANLPIDNKNV | |
| | SNHKKRSVFEYDLVKDKRYTVDKFQFHVPVTINFKSDGNGNINPLVNDYIKKSDDL | |
| | HVIGIDRGERHLLYLTVIDMKGNIKKQFSLNEIVNEYKGNTYSTNYHDLLEKREDKR | |
| | DKERKEWKTIETIKELKEGYLSQVIHKITELMVEYNAIIVLEDLNLGFMRGRQKVEKS | |
| | VYQKFEKMLIDKLNYLADKKKEPEDLGGVLKAYQLANKFESFQKMGKQSGFLFYT | |
| | QAWNTSKIDPVTGFVNLFDTHYENILKSKNFFSKFDLIKYNSDKDWFEFSFDYNNF | |
| | TTKAEGTKTKWTLCTFGNRIISFRNPDNNMQWDGKEINLTEEFKLFFEKFGININSD | |
| | LHAEILKQDKKDFFEGLLHLLKLTLQMRNSKTRTDIDYMQSPVADENGVLYNSNKC | |
| | GKSLPENADANGAYNIARKGLMIIDKIKKSDNLNKIDLTISNKEWLVFAQNKPYLKN | |
| Expression | MGSGKAFENFTGLYPLSKTLRFELKPIGKTLEYIEKHGILDKDKHRANSYVKVKDIID | 117 |
| construct (with | RYHKQFIEDSLSDSDFKLKYENKGKKESLEEYFYYYKLRNRDDKQKKDFDEIQKNL | |
| N-terminal | RKQIASQLKKQDRFKRIDKKELIKEDLLEFVSDDNERNLINEFKDFTTYFTGFHENR | |
| methionine, | QNMYSDEAKSTAIAYRLIHENLPKFIDNISVFERVAATDVADCFAQIYSDFEEYLNVN | |
| V5-tag and C- | DISEIFRLDYYTEILTQTQIDAYNLIIGGRSEGNIKIKGLNEYINLYNQQQKDKSQRLP | |
| terminal NLS) | KLKSLFKQILSDRNAISWLPESFENDNQLLEKLESCYQSFNETYDDKKSIFVRFREL | |
| aa sequence | LLTISDYEMDKIFLRNDLQLTDISQKMFGSYSIISRSLLEDLKRGTSRKSKKETDESF | |
| | EERLRNIIKNQDSFAIGTIDSSLQQMDVEEYKKSICDYFPNLSVDDKGDDIFDRIVKA | |
| | YSEVKDLLNSPYPSDKNLAQEDDDIDKIKNLLESMKDLQKFVKPLCGKGNESDKDE | |
| | RFYGEFTALYEELDKITPLYNMVRNYLTRKPYSTEKIKLNFDNAQLLNGWDLNKES | |
| | DNTSVILRKDGLYYLAIMNKKHNKVFEKNKLQSDGVCFEKMEYKLLPGANKMLPKV | |
| | FFSKSRIDEFGPSQRLLDSYQNETHKKGDKFNIEDCHELIDFFKRSIDKHEDWSKFS | |
| | FSFSDTKTYEDLSGFYREVEHQGYILSFVNVSVDYVNSLVDEGKIYLFQIYNKDFSP | |
| | FSKGTPNMHTLYWKMLFDEENLKDVVYKLNGQAEVFFRKSSIKYDKPTHPANLPID | |
| | NKNVSNHKKRSVFEYDLVKDKRYTVDKFQFHVPVTINFKSDGNGNINPLVNDYIKK | |
| | SDDLHVIGIDRGERHLLYLTVIDMKGNIKKQFSLNEIVNEYKGNTYSTNYHDLLEKR | |
| | EDKRDKERKEWKTIETIKELKEGYLSQVIHKITELMVEYNAIIVLEDLNLGFMRGRQK | |
| | VEKSVYQKFEKMLIDKLNYLADKKKEPEDLGGVLKAYQLANKFESFQKMGKQSGF | |
| | LFYTQAWNTSKIDPVTGFVNLFDTHYENILKSKNFFSKFDLIKYNSDKDWFEFSFDY | |
| | NNFTTKAEGTKTKWTLCTFGNRIISFRNPDNNMQWDGKEINLTEEFKLFFEKFGINI | |
| | NSDLHAEILKQDKKDFFEGLLHLLKLTLQMRNSKTRTDIDYMQSPVADENGVLYNS | |
| | NKCGKSLPENADANGAYNIARKGLMIIDKIKKSDNLNKIDLTISNKEWLVFAQNKPYL | |
| | KNSRKRTADGSEFESPKKKRKVGSGKPIPNPLLGLDST | |
| Wildtype | ATGAAAGCATTTGAGAATTTTACAGGATTGTATCCTCTTTCTAAAACATTAAGAT | 118 |
| coding | TTGAGCTGAAACCGATTGGAAAGACATTGGAATATATTGAGAAGCATGGTATTC | |
| sequence (with | TTGATAAGGATAAACACAGAGCAAATAGTTATGTTAAGGTCAAGGATATAATTG | |
| N-terminal | ACAGATATCATAAACAATTTATTGAAGACTCGTTAAGTGATAGTGATTTTAAACT | |
| methionine | TAAATATGAAAACAAAGGAAAGAAAGAATCATTAGAAGAATATTTCTATTATTAT | |
| and stop | AAATTAAGAAATAGAGACGACAAACAGAAGAAAGATTTTGATGAAATTCAAAAG | |
| codon) | AATCTTAGAAAACAGATTGCAAGTCAATTAAAGAAACAAGATCGTTTTAAAAGAA | |
| | TTGATAAAAAGGAACTTATAAAGGAAGATCTTTTAGAATTTGTTAGTGATGATAA | |
| | TGAAAGGAATCTTATTAATGAATTTAAAGATTTCACGACATATTTTACAGGTTTT | |
| | CACGAAAACAGACAAAATATGTATTCTGATGAAGCCAAATCAACTGCGATAGCG | |
| | TATAGACTGATACATGAGAATCTTCCTAAATTTATAGATAACATTTCAGTTTTTGA | |
| | AAGAGTTGCTGCTACAGATGTGGCTGATTGTTTTGCACAAATCTATTCTGATTTT | |
| | GAGGAATATCTGAATGTAAATGATATATCTGAAATTTTTAGATTAGACTATTATA | |
| | CGGAAATATTAACTCAGACACAGATTGATGCTTATAATCTGATAATTGGAGGAC | |
| | GTTCTGAGGGCAATATTAAAATAAAAGGTTTGAACGAATATATTAATCTGTATAA | |
| | TCAACAGCAGAAAGACAAGTCTCAACGGTTGCCAAAACTGAAGTCTTTGTTTAA | |
| | ACAGATTTTGAGTGATAGAAATGCTATATCTTGGTTGCCAGAATCGTTTGAAAAT | |
| | GATAATCAACTCTTGGAAAAGTTGGAGAGTTGTTATCAGTCTTTTAATGAAACAT | |
| | ATGACGATAAGAAGTCAATATTTGTAAGGTTTAGAGAATTATTGTTGACTATATC | |
| | TGATTATGAAATGGATAAAATATTTCTTCGTAATGATTTGCAGTTGACAGATATT | |
| | TCACAAAAGATGTTCGGTAGTTATAGTATTATTTCAAGGTCTTTATTGGAAGATT | |
| | TAAAGAGAGGTACATCTCGTAAATCAAAGAAGGAAACTGATGAAAGTTTTGAAG | |
| | AAAGGTTGAGAAATATTATCAAAAACCAAGATAGTTTTGCCATTGGAACAATAG | |
| | ATTCGTCTTTGCAACAAATGGATGTTGAAGAATACAAGAAATCTATTTGTGATTA | |
| | TTTCCCTAATTTATCTGTTGATGACAAAGGAGATGATATTTTTGATAGAATAGTA | |
| | AAAGCGTATTCGGAGGTTAAAGACTTGTTGAATTCTCCGTATCCGTCAGATAAA | |
| | AACCTTGCTCAAGAAGATGATGATATTGATAAGATTAAAAATCTTTTAGAGTCAA | |
| | TGAAAGATCTTCAGAAGTTTGTGAAACCTCTCTGTGGAAAAGGAAATGAATCTG | |
| | ATAAAGATGAGCGTTTCTATGGTGAGTTTACGGCTTTATATGAAGAATTAGACA | |
| | AGATAACACCATTATATAATATGGTGAGAAATTATCTTACTCGCAAACCGTATTC | |
| | TACGGAAAAGATAAAGTTAAACTTTGACAATGCTCAACTTTTGAATGGATGGGA | |
| | TTTAAATAAAGAAAGTGATAATACGAGTGTCATATTGCGTAAAGACGGATTGTAT | |
| | TATCTTGCCATCATGAACAAGAAGCATAATAAAGTCTTCGAGAAAAATAAATTAC | |
| | AGTCAGATGGTGTTTGCTTTGAAAAAATGGAGTATAAATTACTTCCTGGTGCAA | |
| | ACAAGATGCTTCCAAAAGTTTTCTTCTCTAAATCAAGGATAGATGAGTTTGGAC | |
| | CTTCTCAAAGATTGTTGGACAGTTATCAGAATGAAACTCATAAAAAAGGTGATA | |
| | AATTCAATATTGAAGATTGCCATGAATTGATAGATTTTTTCAAAAGGTCTATTGA | |
| | TAAACATGAGGATTGGAGTAAATTTAGCTTTAGTTTCTCAGATACTAAGACATAT | |
| | GAAGATTTAAGCGGATTTTACAGAGAAGTTGAGCATCAGGGTTATATACTTTCT | |
| | TTTGTAAATGTTTCTGTAGATTATGTAAATAGTTTGGTAGATGAAGGAAAGATAT | |
| | ATTTATTTCAAATTTATAATAAAGATTTCTCGCCATTTAGCAAAGGAACTCCAAAT | |
| | ATGCATACTTTGTATTGGAAAATGCTTTTTGATGAAGAAAATCTGAAAGATGTGG | |
| | TGTATAAATTGAATGGTCAGGCAGAAGTGTTTTTCAGGAAATCCAGTATAAAGT | |
| | ATGATAAACCGACTCATCCTGCTAATTTGCCTATTGATAATAAAAATGTATCTAA | |
| | CCATAAGAAACGGAGTGTCTTTGAGTATGATTTGGTCAAAGATAAGAGATATAC | |
| | GGTTGATAAATTCCAGTTTCATGTTCCTGTAACAATCAATTTTAAAAGTGATGGA | |
| | AATGGAAATATCAATCCTCTCGTCAATGATTATATCAAAAAGTCTGATGATTTGC | |
| | ATGTGATTGGTATCGACAGGGGAGAGCGTCATCTTTTGTATCTTACGGTCATAG | |
| | ATATGAAAGGTAATATCAAGAAGCAGTTTTCATTGAATGAAATCGTCAATGAATA | |
| | TAAAGGAAATACATATAGTACCAATTATCATGATTTGTTGGAAAAACGCGAGGA | |
| | CAAACGTGATAAGGAAAGAAAAGAATGGAAAACTATAGAAACCATCAAGGAGTT | |
| | GAAAGAAGGTTATCTCAGCCAGGTTATTCATAAAATAACGGAATTGATGGTTGA | |
| | ATATAATGCAATCATTGTGCTGGAGGATCTTAATTTAGGATTTATGCGTGGGCG | |
| | ACAAAAGGTGGAGAAGTCTGTTTATCAAAAGTTTGAAAAGATGTTGATTGATAA | |
| | ACTGAATTATCTTGCTGATAAAAAGAAAGAACCGGAAGATTTGGGTGGTGTGTT | |
| | GAAGGCATATCAACTGGCAAATAAGTTTGAAAGTTTTCAAAAAATGGGAAAACA | |
| | ATCAGGTTTCTTATTCTATACCCAAGCATGGAATACAAGTAAGATAGATCCGGT | |
| | TACTGGTTTTGTTAATCTTTTTGACACACATTATGAGAATATCTTAAAGTCTAAAA | |
| | ATTTCTTCTCTAAGTTTGATTTGATAAAGTATAATTCTGATAAAGATTGGTTCGA | |
| | GTTTTCTTTTGATTATAATAATTTTACAACTAAAGCAGAAGGTACAAAAACAAAAT | |
| | GGACATTATGTACCTTTGGAAATAGAATAATATCATTCCGTAATCCTGATAATAA | |
| | TATGCAATGGGATGGAAAAGAAATTAATCTTACTGAAGAATTCAAGTTATTCTTT | |
| | GAGAAATTTGGAATCAATATTAATTCTGATTTGCATGCGGAAATATTAAAACAAG | |
| | ATAAAAAAGACTTCTTTGAAGGTCTTTTGCATTTGTTGAAATTGACATTGCAGAT | |
| | GCGTAATAGTAAGACTCGCACTGATATAGATTATATGCAGTCTCCTGTAGCAGA | |
| | CGAAAACGGAGTGTTATACAATAGTAATAAATGTGGTAAATCCTTGCCAGAAAA | |
| | TGCTGATGCTAACGGTGCGTATAATATTGCAAGAAAAGGTCTTATGATAATTGA | |
| | CAAAATAAAGAAGTCTGATAATCTGAATAAAATAGATCTTACGATCTCTAATAAG | |
| | GAGTGGTTGGTATTCGCACAAAATAAACCATATTTGAAGAATTGA | |
| Codon | AAGGCCTTCGAGAACTTCACCGGCCTGTATCCCCTCTCTAAAACCCTGAGATTT | 119 |
| optimized | GAGCTGAAGCCAATCGGCAAGACCCTCGAATACATTGAGAAGCACGGCATCCT | |
| coding | GGACAAGGACAAGCACAGAGCCAATAGCTACGTGAAGGTGAAGGACATCATC | |
| sequence (no | GACAGATACCACAAACAGTTCATCGAGGACTCTCTGTCTGATAGCGACTTCAA | |
| N-terminal | GCTAAAGTACGAGAACAAAGGCAAGAAGGAGAGCCTGGAAGAGTACTTCTACT | |
| methionine, no | ACTACAAGCTGCGGAACCGGGATGATAAGCAAAAGAAAGATTTTGATGAGATC | |
| stop codon) | CAGAAGAACCTGAGAAAACAAATCGCCAGCCAGCTCAAAAAACAGGACAGATT | |
| | CAAGCGGATCGACAAGAAAGAACTGATCAAGGAAGATCTGCTGGAGTTCGTGA | |
| | GCGACGACAATGAAAGAAACCTGATCAACGAGTTCAAGGATTTTACTACATACT | |
| | TTACCGGCTTCCACGAGAACCGGCAGAACATGTACTCTGATGAGGCCAAGTCC | |
| | ACCGCCATCGCTTATAGACTGATTCACGAGAATCTGCCTAAGTTCATCGATAAC | |
| | ATAAGCGTGTTCGAGCGGGTCGCAGCTACAGATGTGGCCGACTGCTTCGCCC | |
| | AGATCTACTCCGATTTCGAGGAATACCTGAACGTGAACGACATCAGCGAGATC | |
| | TTCAGACTGGACTACTATACAGAAATCCTGACCCAGACCCAGATCGACGCCTA | |
| | CAATCTGATCATTGGCGGCAGAAGCGAGGGCAACATCAAAATTAAAGGCTTGA | |
| | ACGAGTACATCAATCTGTACAACCAGCAGCAGAAAGACAAGAGCCAAAGACTG | |
| | CCCAAGCTGAAGAGCCTGTTTAAACAGATCCTGAGCGACAGAAATGCCATATC | |
| | TTGGTTGCCTGAGTCTTTCGAGAACGATAACCAGCTGCTGGAGAAGCTGGAGA | |
| | GCTGCTACCAGAGCTTCAACGAAACCTACGACGACAAGAAGTCTATCTTTGTTA | |
| | GATTTAGAGAACTGCTGCTGACAATCTCTGACTACGAGATGGACAAAATCTTCC | |
| | TGAGAAATGACCTGCAGCTGACCGACATCTCCCAAAAAATGTTCGGATCTTACA | |
| | GCATCATCTCCCGGAGCCTGTTAGAGGATCTCAAGAGAGGAACCAGCCGGAA | |
| | GTCAAAGAAGGAAACAGACGAGAGCTTCGAAGAACGGCTGCGCAACATTATCA | |
| | AGAATCAGGACTCCTTTGCCATCGGCACCATCGATAGCAGCCTGCAGCAGATG | |
| | GACGTGGAAGAGTACAAGAAATCCATCTGCGACTATTTCCCTAATCTGAGTGTT | |
| | GACGACAAGGGCGATGACATATTTGACAGAATCGTGAAAGCCTATAGCGAGGT | |
| | GAAGGACCTGCTGAACTCCCCTTACCCTAGCGACAAGAACCTGGCTCAGGAG | |
| | GACGACGACATCGACAAGATCAAAAACCTGCTGGAAAGCATGAAGGACCTGCA | |
| | GAAGTTCGTCAAGCCTCTGTGTGGCAAGGGCAACGAGAGCGATAAGGATGAA | |
| | AGGTTCTACGGCGAGTTCACAGCCCTGTACGAGGAACTGGACAAGATCACCCC | |
| | TCTGTACAATATGGTGCGGAACTACCTGACAAGAAAGCCATACTCTACCGAGA | |
| | AGATCAAACTGAACTTCGACAACGCCCAGCTGCTGAACGGATGGGACCTGAAT | |
| | AAAGAGAGCGACAACACCAGCGTCATCCTGCGTAAGGATGGCCTGTACTACCT | |
| | GGCCATCATGAACAAGAAGCACAACAAGGTGTTCGAGAAGAACAAGCTCCAAA | |
| | GCGATGGCGTGTGCTTCGAGAAGATGGAGTACAAGCTGCTGCCTGGCGCCAA | |
| | CAAGATGCTGCCAAAGGTGTTCTTCTCTAAGAGCAGAATCGATGAGTTCGGCC | |
| | CTTCTCAGAGACTGCTGGACAGCTACCAGAACGAAACCCACAAGAAGGGCGA | |
| | CAAATTCAACATCGAGGACTGTCACGAGCTGATCGACTTTTTCAAAAGAAGCAT | |
| | CGACAAACATGAAGATTGGAGCAAGTTTTCTTTTAGCTTCAGCGACACCAAGAC | |
| | CTACGAGGACCTGAGCGGCTTCTACAGAGAAGTAGAACACCAGGGCTACATCC | |
| | TGAGCTTTGTGAACGTGAGCGTGGATTACGTGAACAGCCTGGTGGACGAGGG | |
| | AAAGATCTACTTATTTCAGATCTACAACAAGGATTTCAGCCCTTTCTCTAAGGG | |
| | CACCCCTAACATGCACACACTGTACTGGAAGATGCTGTTCGACGAGGAAAACC | |
| | TGAAGGATGTGGTGTACAAGCTGAATGGCCAGGCCGAAGTGTTCTTCAGAAAG | |
| | TCCTCTATCAAGTACGACAAACCTACCCATCCTGCCAATCTCCCCATCGATAAC | |
| | AAGAACGTGAGCAACCACAAGAAGCGGAGCGTGTTCGAGTACGACCTGGTGA | |
| | AGGACAAACGTTACACCGTGGATAAGTTCCAGTTCCACGTGCCCGTGACCATC | |
| | AACTTCAAGAGCGATGGCAACGGCAATATCAACCCCCTGGTGAACGACTACAT | |
| | CAAGAAGAGCGACGATCTACACGTGATCGGCATCGACAGAGGAGAACGGCAC | |
| | CTGCTGTACCTGACGGTGATCGACATGAAGGGCAACATCAAGAAACAATTTAG | |
| | CCTGAACGAGATCGTGAACGAATATAAGGGCAATACCTACAGCACCAACTACC | |
| | ACGACCTGCTGGAGAAACGGGAAGATAAGAGAGATAAGGAGAGAAAGGAATG | |
| | GAAAACCATTGAAACAATCAAGGAACTGAAAGAAGGATATCTGAGCCAGGTGA | |
| | TCCACAAGATCACCGAGCTGATGGTGGAGTACAACGCCATCATCGTCCTGGAG | |
| | GACCTGAACCTGGGCTTCATGAGAGGGAGACAGAAGGTGGAGAAGTCCGTAT | |
| | ACCAGAAATTTGAAAAGATGCTGATCGACAAGCTGAACTACCTGGCTGACAAG | |
| | AAAAAGGAACCTGAGGACCTTGGAGGCGTCCTGAAGGCCTACCAGCTGGCCA | |
| | ACAAATTCGAATCTTTCCAAAAGATGGGCAAACAGAGCGGCTTTCTGTTTTACA | |
| | CCCAGGCTTGGAACACCAGCAAGATCGACCCCGTGACGGGCTTCGTGAACCT | |
| | CTTCGATACACATTACGAGAACATCCTGAAGAGCAAGAATTTCTTCAGCAAGTT | |
| | CGATCTCATCAAATATAACAGCGATAAAGATTGGTTCGAGTTCTCGTTCGACTA | |
| | CAACAATTTCACCACCAAGGCCGAGGGCACCAAAACAAAGTGGACACTGTGCA | |
| | CCTTCGGAAACAGAATCATCAGCTTTAGAAACCCTGACAACAACATGCAGTGG | |
| | GATGGCAAGGAGATCAACCTGACAGAGGAGTTCAAGCTGTTCTTCGAGAAGTT | |
| | CGGCATCAACATCAACTCCGACCTGCACGCTGAGATCCTGAAGCAAGACAAGA | |
| | AGGACTTCTTCGAGGGCCTGCTGCACCTGCTGAAACTGACACTCCAGATGCGG | |
| | AACAGCAAGACGAGGACCGATATCGACTACATGCAGAGCCCCGTGGCCGACG | |
| | AGAATGGGGTGCTGTACAACTCCAACAAATGCGGCAAGAGCCTGCCCGAGAA | |
| | CGCCGATGCCAACGGAGCCTACAACATCGCTAGAAAGGGACTGATGATCATTG | |
| | ACAAGATCAAGAAGTCTGACAACCTGAACAAGATCGATCTGACTATCTCTAACA | |
| | AGGAATGGCTGGTGTTCGCCCAGAACAAGCCTTACCTGAAAAAT | |
| Expression | ATGggctccggaAAGGCCTTCGAGAACTTCACCGGCCTGTATCCCCTCTCTAAAAC | 120 |
| construct (with | CCTGAGATTTGAGCTGAAGCCAATCGGCAAGACCCTCGAATACATTGAGAAGC | |
| N-terminal | ACGGCATCCTGGACAAGGACAAGCACAGAGCCAATAGCTACGTGAAGGTGAA | |
| methionine | GGACATCATCGACAGATACCACAAACAGTTCATCGAGGACTCTCTGTCTGATA | |
| and stop | GCGACTTCAAGCTAAAGTACGAGAACAAAGGCAAGAAGGAGAGCCTGGAAGA | |
| codon, | GTACTTCTACTACTACAAGCTGCGGAACCGGGATGATAAGCAAAAGAAAGATTT | |
| includes V5- | TGATGAGATCCAGAAGAACCTGAGAAAACAAATCGCCAGCCAGCTCAAAAAAC | |
| tag and C- | AGGACAGATTCAAGCGGATCGACAAGAAAGAACTGATCAAGGAAGATCTGCTG | |
| terminal NLS) | GAGTTCGTGAGCGACGACAATGAAAGAAACCTGATCAACGAGTTCAAGGATTT | |
| | TACTACATACTTTACCGGCTTCCACGAGAACCGGCAGAACATGTACTCTGATGA | |
| | GGCCAAGTCCACCGCCATCGCTTATAGACTGATTCACGAGAATCTGCCTAAGT | |
| | TCATCGATAACATAAGCGTGTTCGAGCGGGTCGCAGCTACAGATGTGGCCGAC | |
| | TGCTTCGCCCAGATCTACTCCGATTTCGAGGAATACCTGAACGTGAACGACAT | |
| | CAGCGAGATCTTCAGACTGGACTACTATACAGAAATCCTGACCCAGACCCAGA | |
| | TCGACGCCTACAATCTGATCATTGGCGGCAGAAGCGAGGGCAACATCAAAATT | |
| | AAAGGCTTGAACGAGTACATCAATCTGTACAACCAGCAGCAGAAAGACAAGAG | |
| | CCAAAGACTGCCCAAGCTGAAGAGCCTGTTTAAACAGATCCTGAGCGACAGAA | |
| | ATGCCATATCTTGGTTGCCTGAGTCTTTCGAGAACGATAACCAGCTGCTGGAG | |
| | AAGCTGGAGAGCTGCTACCAGAGCTTCAACGAAACCTACGACGACAAGAAGTC | |
| | TATCTTTGTTAGATTTAGAGAACTGCTGCTGACAATCTCTGACTACGAGATGGA | |
| | CAAAATCTTCCTGAGAAATGACCTGCAGCTGACCGACATCTCCCAAAAAATGTT | |
| | CGGATCTTACAGCATCATCTCCCGGAGCCTGTTAGAGGATCTCAAGAGAGGAA | |
| | CCAGCCGGAAGTCAAAGAAGGAAACAGACGAGAGCTTCGAAGAACGGCTGCG | |
| | CAACATTATCAAGAATCAGGACTCCTTTGCCATCGGCACCATCGATAGCAGCCT | |
| | GCAGCAGATGGACGTGGAAGAGTACAAGAAATCCATCTGCGACTATTTCCCTA | |
| | ATCTGAGTGTTGACGACAAGGGCGATGACATATTTGACAGAATCGTGAAAGCC | |
| | TATAGCGAGGTGAAGGACCTGCTGAACTCCCCTTACCCTAGCGACAAGAACCT | |
| | GGCTCAGGAGGACGACGACATCGACAAGATCAAAAACCTGCTGGAAAGCATG | |
| | AAGGACCTGCAGAAGTTCGTCAAGCCTCTGTGTGGCAAGGGCAACGAGAGCG | |
| | ATAAGGATGAAAGGTTCTACGGCGAGTTCACAGCCCTGTACGAGGAACTGGAC | |
| | AAGATCACCCCTCTGTACAATATGGTGCGGAACTACCTGACAAGAAAGCCATA | |
| | CTCTACCGAGAAGATCAAACTGAACTTCGACAACGCCCAGCTGCTGAACGGAT | |
| | GGGACCTGAATAAAGAGAGCGACAACACCAGCGTCATCCTGCGTAAGGATGG | |
| | CCTGTACTACCTGGCCATCATGAACAAGAAGCACAACAAGGTGTTCGAGAAGA | |
| | ACAAGCTCCAAAGCGATGGCGTGTGCTTCGAGAAGATGGAGTACAAGCTGCTG | |
| | CCTGGCGCCAACAAGATGCTGCCAAAGGTGTTCTTCTCTAAGAGCAGAATCGA | |
| | TGAGTTCGGCCCTTCTCAGAGACTGCTGGACAGCTACCAGAACGAAACCCACA | |
| | AGAAGGGCGACAAATTCAACATCGAGGACTGTCACGAGCTGATCGACTTTTTC | |
| | AAAAGAAGCATCGACAAACATGAAGATTGGAGCAAGTTTTCTTTTAGCTTCAGC | |
| | GACACCAAGACCTACGAGGACCTGAGCGGCTTCTACAGAGAAGTAGAACACCA | |
| | GGGCTACATCCTGAGCTTTGTGAACGTGAGCGTGGATTACGTGAACAGCCTGG | |
| | TGGACGAGGGAAAGATCTACTTATTTCAGATCTACAACAAGGATTTCAGCCCTT | |
| | TCTCTAAGGGCACCCCTAACATGCACACACTGTACTGGAAGATGCTGTTCGAC | |
| | GAGGAAAACCTGAAGGATGTGGTGTACAAGCTGAATGGCCAGGCCGAAGTGT | |
| | TCTTCAGAAAGTCCTCTATCAAGTACGACAAACCTACCCATCCTGCCAATCTCC | |
| | CCATCGATAACAAGAACGTGAGCAACCACAAGAAGCGGAGCGTGTTCGAGTAC | |
| | GACCTGGTGAAGGACAAACGTTACACCGTGGATAAGTTCCAGTTCCACGTGCC | |
| | CGTGACCATCAACTTCAAGAGCGATGGCAACGGCAATATCAACCCCCTGGTGA | |
| | ACGACTACATCAAGAAGAGCGACGATCTACACGTGATCGGCATCGACAGAGGA | |
| | GAACGGCACCTGCTGTACCTGACGGTGATCGACATGAAGGGCAACATCAAGAA | |
| | ACAATTTAGCCTGAACGAGATCGTGAACGAATATAAGGGCAATACCTACAGCA | |
| | CCAACTACCACGACCTGCTGGAGAAACGGGAAGATAAGAGAGATAAGGAGAG | |
| | AAAGGAATGGAAAACCATTGAAACAATCAAGGAACTGAAAGAAGGATATCTGA | |
| | GCCAGGTGATCCACAAGATCACCGAGCTGATGGTGGAGTACAACGCCATCATC | |
| | GTCCTGGAGGACCTGAACCTGGGCTTCATGAGAGGGAGACAGAAGGTGGAGA | |
| | AGTCCGTATACCAGAAATTTGAAAAGATGCTGATCGACAAGCTGAACTACCTGG | |
| | CTGACAAGAAAAAGGAACCTGAGGACCTTGGAGGCGTCCTGAAGGCCTACCA | |
| | GCTGGCCAACAAATTCGAATCTTTCCAAAAGATGGGCAAACAGAGCGGCTTTC | |
| | TGTTTTACACCCAGGCTTGGAACACCAGCAAGATCGACCCCGTGACGGGCTTC | |
| | GTGAACCTCTTCGATACACATTACGAGAACATCCTGAAGAGCAAGAATTTCTTC | |
| | AGCAAGTTCGATCTCATCAAATATAACAGCGATAAAGATTGGTTCGAGTTCTCG | |
| | TTCGACTACAACAATTTCACCACCAAGGCCGAGGGCACCAAAACAAAGTGGAC | |
| | ACTGTGCACCTTCGGAAACAGAATCATCAGCTTTAGAAACCCTGACAACAACAT | |
| | GCAGTGGGATGGCAAGGAGATCAACCTGACAGAGGAGTTCAAGCTGTTCTTCG | |
| | AGAAGTTCGGCATCAACATCAACTCCGACCTGCACGCTGAGATCCTGAAGCAA | |
| | GACAAGAAGGACTTCTTCGAGGGCCTGCTGCACCTGCTGAAACTGACACTCCA | |
| | GATGCGGAACAGCAAGACGAGGACCGATATCGACTACATGCAGAGCCCCGTG | |
| | GCCGACGAGAATGGGGTGCTGTACAACTCCAACAAATGCGGCAAGAGCCTGC | |
| | CCGAGAACGCCGATGCCAACGGAGCCTACAACATCGCTAGAAAGGGACTGAT | |
| | GATCATTGACAAGATCAAGAAGTCTGACAACCTGAACAAGATCGATCTGACTAT | |
| | CTCTAACAAGGAATGGCTGGTGTTCGCCCAGAACAAGCCTTACCTGAAAAATtct | |
| | agaAAGCGGACAGCAGACGGCTCCGAATTTGAAAGCCCTAAGAAAAAGAGAAA | |
| | GGTGggatccGGCAAACCTATCCCCAATCCCCTGCTGGGCCTGGACAGCACCTG | |
| | A | |

In some embodiments a ZRXE Type V Cas protein comprises an amino acid sequence of SEQ ID NO: 115, SEQ ID NO: 116, or SEQ ID NO: 117. In some embodiments, a ZRXE Type V Cas protein has nickase activity, for example resulting from one or more amino acid substitutions relative to the sequence of SEQ ID NO: 115, SEQ ID NO: 116, or SEQ ID NO: 117. In some embodiments, the one or more amino acid substitutions providing nickase activity comprise a D862 substitution, wherein the position of the D862 substitution is defined with respect to the amino acid numbering of SEQ ID NO: 116 (corresponding to amino acid 908 of SEQ ID NO: 121). In some embodiments, the one or more amino acid substitutions providing nickase activity comprise an E955 substitution, wherein the position of the E955 substitution is defined with respect to the amino acid numbering of SEQ ID NO: 116 (corresponding to amino acid 993 of SEQ ID NO: 121). In some embodiments, the one or more amino acid substitutions providing nickase activity comprise a R1167 substitution, wherein the position of the R1167 substitution is defined with respect to the amino acid numbering of SEQ ID NO: 116 (corresponding to amino acid 1226 of SEQ ID NO: 121). In some embodiments, the one or more amino acid substitutions providing nickase activity comprise a D1204 substitution, wherein the position of the D1204 substitution is defined with respect to the amino acid numbering of SEQ ID NO: 116 (corresponding to amino acid 1263 of SEQ ID NO: 121). In some embodiments, a ZRXE Type V Cas protein is catalytically inactive, for example due to a R1167 substitution in combination with a D862 substitution, a E955 substitution, and/or D1204 substitution.

### 6.2.21. Fusion and Chimeric Proteins

The disclosure provides Type V Cas proteins, e.g., a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, and a ZRXE Type V Cas protein, which are in the form of fusion proteins comprising a Type V Cas protein sequence fused with one or more additional amino acid sequences, such as one or more nuclear localization signals and/or one or more non-native tags. Fusion proteins can also comprise an amino acid sequence of, for example, a nucleoside deaminase, a reverse transcriptase, a transcriptional activator (e.g., VP64), a transcriptional repressor (e.g., Krüppel associated box (KRAB)), a histone-modifying protein, an integrase, or a recombinase. Fusion proteins can include linker sequences joining different portions of the fusion protein. For example, glycine-serine linkers such as GS, SG, or GS or SG repeats, (e.g., GSGS (SEQ ID NO:259)). In some embodiments, one or more fusion partners (e.g., an adenosine deaminase or cytidine deaminase) is/are positioned N-terminal to a Type V Cas protein sequence. In some embodiments, one or more fusion partners (e.g., an adenosine deaminase or cytidine deaminase) is/are positioned C-terminal to a Type V Cas protein sequence.

In some embodiments, a fusion protein of the disclosure comprises a means for localizing the Type V Cas protein to the nucleus, for example a nuclear localization signal.

Non-limiting examples of nuclear localization signals include KRTADGSEFESPKKKRKV (SEQ ID NO: 122), PKKKRKV (SEQ ID NO: 123), PKKKRRV (SEQ ID NO: 124), KRPAATKKAGQAKKKK (SEQ ID NO: 125), YGRKKRRQRRR (SEQ ID NO: 126), RKKRRQRRR (SEQ ID NO: 127), PAAKRVKLD (SEQ ID NO: 128), RQRRNELKRSP (SEQ ID NO: 129), VSRKRPRP (SEQ ID NO: 130), PPKKARED (SEQ ID NO: 131), PQPKKKPL (SEQ ID NO: 132), SALIKKKKKMAP (SEQ ID NO: 133), PKQKKRK (SEQ ID NO: 134), RKLKKKIKKL (SEQ ID NO: 135), REKKKFLKRR (SEQ ID NO: 136), KRKGDEVDGVDEVAKKKSKK (SEQ ID NO: 137), RKCLQAGMNLEARKTKK (SEQ ID NO:138), NQSSNFGPMKGGNFGGRSSGPYGGGGQYFAKPRNQGGY (SEQ ID NO: 139), RMRIZFKNKGKDTAELRRRRVEVSVELRKAKKDEQILKRRNV (SEQ ID NO:140), and SSDDEATADSQHAAPPKKKRKV (SEQ ID NO: 178). Additional non-limiting examples of nuclear localization signals include PKKKRKVG (SEQ ID NO:179) and GRSSDDEATADSQHAAPPKKKRKV (SEQ ID NO:180).

Exemplary fusion partners include protein tags (e.g., V5-tag (e.g., having the sequence GKPIPNPLLGLDST (SEQ ID NO:141) or IPNPLLGLD (SEQ ID NO:142)), FLAG-tag, myc-tag, HA-tag, GST-tag, polyHis-tag, MBP-tag), protein domains, transcription modulators, enzymes acting on small molecule substrates, DNA, RNA and protein modification enzymes (e.g., adenosine deaminase, cytidine deaminase, guanosyl transferase, DNA methyltransferase, RNA methyltransferases, DNA demethylases, RNA demethylases, dioxygenases, polyadenylate polymerases, pseudouridine synthases, acetyltransferases, deacetylase, ubiquitin-ligases, deubiquitinases, kinases, phosphatases, NEDD8-ligases, de-NEDDylases, SUMO-ligases, deSUMOylases, histone deacetylases, reverse transcriptases, histone acetyltransferases histone methyltransferases, histone demethylases), protein DNA binding domains, RNA binding proteins, polypeptide sequences with specific biological functions (e.g., nuclear localization signals, mitochondrial localization signals, plastid localization signals, subcellular localization signals, destabilizing signals, Geminin destruction box motifs), and biological tethering domains (e.g., MS2, Csy4 and lambda N protein). Various Type V Cas fusion proteins are described in Ribeiro et al., 2018, In. J. Genomics, Article ID: 1652567; Jayavaradhan, et al., 2019, Nat Commun 10:2866; Xiao et al., 2019, The CRISPR Journal, 2 (1): 51-63; Mali et al., 2013, Nat Methods. 10 (10): 957-63; U.S. Pat. Nos. 9,322,037, and 9,388,430. In some embodiments, a fusion partner is an adenosine deaminase. An exemplary adenosine deaminase is the tRNA adenosine deaminase (TadA) moiety contained in the adenine base editor ABE8e (Richter, 2020, Nature Biotechnology 38:883-891). The TadA moiety of ABE8e comprises the following amino acid sequence:

(SEQ ID NO: 143)
SEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHS
RIGRVVFGVRNSKRGAAGSLMNVLNYPGMNHRVEITEGILADECAALL
CDFYRMPRQVFNAQKKAQSSIN

In some embodiments, an adenosine deaminase fusion partner comprises an amino acid sequence having at least 50%, at least 60%, at least 70%, at least 80%, at least 90%, at least 95%, at least 97%, at least 98%, at least 99% amino acid sequence identity with SEQ ID NO: 143.

Type V Cas proteins of the disclosure in the form of a fusion protein comprising an adenosine deaminase can be used, for example, as an adenine base editor (ABE) to change an “A” to a “G” in DNA. Type V Cas proteins of the disclosure in the form of a fusion protein comprising a cytidine deaminase can be used, for example, as a cytosine base editor (CBE) to change a “C” to a “T” in DNA.

In some embodiments, a fusion protein of the disclosure comprises a means for deaminating adenosine, for example an adenosine deaminase, e.g., a TadA variant. In some embodiments, a fusion protein of the disclosure comprises a means for deaminating cytidine, for example a cytidine deaminase, e.g., cytidine deaminase 1 (CDA1) or an apolipoprotein B mRNA-editing complex (APOBEC) family deaminase (see, e.g., Cheng et al., 2019, Nat Commun. 10 (1): 3612; Gehrke et al., 2018, Nat Biotechnol. 36 (10): 977-982; Komor et al., 2016, Nature 533 (7603): 420-424, Porto and Komor, 2023, PLOS Biol 21 (4): e3002071, the contents of each of which are incorporated herein by reference in their entireties).

Exemplary deaminases that can be used in fusion proteins of the disclosure are set forth in Table 2.

TABLE 2

| | | | | Addgene |
|---|---|---|---|---|
| | | | | catalog #/ |
| | | SEQ ID | | DOI |
| Name | Amino Acid Sequence | NO | Note | reference |
| APOBEC1 | SSETGPVAVDPTLRRRIEPHEFEVFFDPRELRKET | 214 | | #87437 |
| | CLLYEINWGGRHSIWRHTSQNTNKHVEVNFIEKFT | | | |
| | TERYFCPNTRCSITWFLSWSPCGECSRAITEFLSR | | | |
| | YPHVTLFIYIARLYHHADPRNRQGLRDLISSGVTIQI | | | |
| | MTEQESGYCWRNFVNYSPSNEAHWPRYPHLWV | | | |
| | RLYVLELYCIILGLPPCLNILRRKQPQLTFFTIALQS | | | |
| | CHYQRLPPHILWATGLK | | | |
| evoAPOBEC | SSKTGPVAVDPTLRRRIEPHEFEVFFDPRELRKET | 215 | APOBEC1 | #122611 |
| | CLLYEINWGGRHSIWRHTSQNTNKHVEVNFIEKFT | | E4K H109N | |
| | TERYFCPNTRCSITWFLSWSPCGECSRAITEFLSR | | H122L | |
| | YPNVTLFIYIARLYHLANPRNRQGLRDLISSGVTIQI | | D124N | |
| | MTEQESGYCWHNFVNYSPSNESHWPRYPHLWV | | R154H | |
| | RLYVLELYCIILGLPPCLNILRRKQSQLTSFTIALQS | | A165S P201S | |
| | CHYQRLPPHILWATGLK | | F205S | |
| YE1 | SSETGPVAVDPTLRRRIEPHEFEVFFDPRELRKET | 216 | APOBEC1 | #138155 |
| | CLLYEINWGGRHSIWRHTSQNTNKHVEVNFIEKFT | | W90Y | |
| | TERYFCPNTRCSITWFLSYSPCGECSRAITEFLSR | | R126E | |
| | YPHVTLFIYIARLYHHADPENRQGLRDLISSGVTIQI | | | |
| | MTEQESGYCWRNFVNYSPSNEAHWPRYPHLWV | | | |
| | RLYVLELYCIILGLPPCLNILRRKQPQLTFFTIALQS | | | |
| | CHYQRLPPHILWATGLK | | | |
| FERNY | SFERNYDPRELRKETYLLYEIKWGKSGKLWRHWC | 217 | | #157944 |
| | QNNRTQHAEVYFLENIFNARRFNPSTHCSITWYLS | | | |
| | WSPCAECSQKIVDFLKEHPNVNLEIYVARLYYHED | | | |
| | ERNRQGLRDLVNSGVTIRIMDLPDYNYCWKTFVS | | | |
| | DQGGDEDYWPGHFAPWIKQYSLKL | | | |
| ppAPOBEC1 | TSEKGPSTGDPTLRRRIESWEFDVFYDPRELRKE | 218 | | #138349 |
| | TCLLYEIKWGMSRKIWRSSGKNTTNHVEVNFIKKF | | | |
| | TSERRFHSSISCSITWFLSWSPCWECSQAIREFLS | | | |
| | QHPGVTLVIYVARLFWHMDQRNRQGLRDLVNSG | | | |
| | VTIQIMRASEYYHCWRNFVNYPPGDEAHWPQYPP | | | |
| | LWMMLYALELHCIILSLPPCLKISRRWQNHLAFFRL | | | |
| | HLQNCHYQTIPPHILLATGLIHPSVTWRLK | | | |
| amAPOBEC1 | ADSSEKMRGQYISRDTFEKNYKPIDGTKEAHLLCE | 219 | | #138342 |
| | IKWGKYGKPWLHWCQNQRMNIHAEDYFMNNIFK | | | |
| | AKKHPVHCYVTWYLSWSPCADCASKIVKFLEERP | | | |
| | YLKLTIYVAQLYYHTEEENRKGLRLLRSKKVIIRVM | | | |
| | DISDYNYCWKVFVSNQNGNEDYWPLQFDPWVKE | | | |
| | NYSRLLDIFWESKCRSPNPW | | | |
| Anc689 | SSETGPVAVDPTLRRRIEPHEFEVFFDPRELRKET | 220 | | #163526 |
| | CLLYEIKWGTSHKIWRHSSKNTTKHVEVNFIEKFT | | | |
| | SERHFCPSTSCSITWFLSWSPCGECSKAITEFLSQ | | | |
| | HPNVTLVIYVARLYHHMDQQNRQGLRDLVNSGVT | | | |
| | IQIMTAPEYDYCWRNFVNYPPGKEAHWPRYPPLW | | | |
| | MKLYALELHAGILGLPPCLNILRRKQPQLTFFTIALQ | | | |
| | SCHYQRLPPHILWATGLK | | | |
| APOBEC | EASPASGPRHLMDPHIFTSNFNNGIGRHKTYLCYE | 221 | | #113410 |
| A3A | VERLDNGTSVKMDQHRGFLHNQAKNLLCGFYGR | | | |
| | HAELRFLDLVPSLQLDPAQIYRVTWFISWSPCFSW | | | |
| | GCAGEVRAFLQENTHVRLRIFAARIYDYDPLYKEA | | | |
| | LQMLRDAGAQVSIMTYDEFKHCWDTFVDHQGCP | | | |
| | FQPWDGLDEHSQALSGRLRAILQNQGN | | | |
| APOBEC3 | EASPASGPRHLMDPHIFTSNFNNGIGRHKTYLCYE | 222 | APOBEC | #131315 |
| eA3A | VERLDNGTSVKMDQHRGFLHGQAKNLLCGFYGR | | A3A N57G | |
| | HAELRFLDLVPSLQLDPAQIYRVTWFISWSPCFSW | | | |
| | GCAGEVRAFLQENTHVRLRIFAARIYDYDPLYKEA | | | |
| | LQMLRDAGAQVSIMTYDEFKHCWDTFVDHQGCP | | | |
| | FQPWDGLDEHSQALSGRLRAILQNQGN | | | |
| APOBEC | NPQIRNPMERMYRDTFYDNFENEPILYGRSYTWL | 223 | | #113411 |
| A3B | CYEVKIKRGRSNLLWDTGVFRGQVYFKPQYHAEM | | | |
| | CFLSWFCGNQLPAYKCFQITWFVSWTPCPDCVAK | | | |
| | LAEFLSEHPNVTLTISAARLYYYWERDYRRALCRL | | | |
| | SQAGARVKIMDYEEFAYCWENFVYNEGQQFMPW | | | |
| | YKFDENYAFLHRTLKEILRYLMDPDTFTFNFNNDP | | | |
| | LVLRRRQTYLCYEVERLDNGTWVLMDQHMGFLC | | | |
| | NEAKNLLCGFYGRHAELRFLDLVPSLQLDPAQIYR | | | |
| | VTWFISWSPCFSWGCAGEVRAFLQENTHVRLRIF | | | |
| | AARIYDYDPLYKEALQMLRDAGAQVSIMTYDEFEY | | | |
| | CWDTFVYRQGCPFQPWDGLEEHSQALSGRLRAI | | | |
| | LQNQGN | | | |
| APOBEC | NPQIRNPMKAMYPGTFYFQFKNLWEANDRNETW | 224 | | #113412 |
| A3C | LCFTVEGIKRRSVVSWKTGVFRNQVDSETHCHAE | | | #119136 |
| | RCFLSWFCDDILSPNTKYQVTWYTSWSPCPDCA | | | |
| | GEVAEFLARHSNVNLTIFTARLYYFQYPCYQEGLR | | | |
| | SLSQEGVAVEIMDYEDFKYCWENFVYNDNEPFKP | | | |
| | WKGLKTNFRLLKRRLRESLQ | | | |
| APOBEC | NPQIRNPMERMYRDTFYDNFENEPILYGRSYTWL | 225 | | #119137 |
| A3D | CYEVKIKRGRSNLLWDTGVFRGPVLPKRQSNHRQ | | | |
| | EVYFRFENHAEMCFLSWFCGNRLPANRRFQITWF | | | |
| | VSWNPCLPCVVKVTKFLAEHPNVTLTISAARLYYY | | | |
| | RDRDWRWVLLRLHKAGARVKIMDYEDFAYCWEN | | | |
| | FVCNEGQPFMPWYKFDDNYASLHRTLKEILRNPM | | | |
| | EAMYPHIFYFHFKNLLKACGRNESWLCFTMEVTK | | | |
| | HHSAVFRKRGVFRNQVDPETHCHAERCFLSWFC | | | |
| | DDILSPNTNYEVTWYTSWSPCPECAGEVAEFLAR | | | |
| | HSNVNLTIFTARLCYFWDTDYQEGLCSLSQEGAS | | | |
| | VKIMGYKDFVSCWKNFVYSDDEPFKPWKGLQTNF | | | |
| | RLLKRRLREILQ | | | |
| APOBEC | KPHFRNTVERMYRDTFSYNFYNRPILSRRNTVWL | 226 | | #119138 |
| A3F | CYEVKTKGPSRPRLDAKIFRGQVYSQPEHHAEMC | | | |
| | FLSWFCGNQLPAYKCFQITWFVSWTPCPDCVAKL | | | |
| | AEFLAEHPNVTLTISAARLYYYWERDYRRALCRLS | | | |
| | QAGARVKIMDDEEFAYCWENFVYSEGQPFMPWY | | | |
| | KFDDNYAFLHRTLKEILRNPMEAMYPHIFYFHFKN | | | |
| | LRKAYGRNESWLCFTMEVVKHHSPVSWKRGVFR | | | |
| | NQVDPETHCHAERCFLSWFCDDILSPNTNYEVTW | | | |
| | YTSWSPCPECAGEVAEFLARHSNVNLTIFTARLYY | | | |
| | FWDTDYQEGLRSLSQEGASVEIMGYKDFKYCWE | | | |
| | NFVYNDDEPFKPWKGLKYNFLFLDSKLQEILE | | | |
| APOBEC | KPHFRNTVERMYRDTFSYNFYNRPILSRRNTVWL | 227 | | #119139 |
| A3G | CYEVKTKGPSRPPLDAKIFRGQVYSELKYHPEMR | | | |
| | FFHWFSKWRKLHRDQEYEVTWYISWSPCTKCTR | | | |
| | DMATFLAEDPKVTLTIFVARLYYFWDPDYQEALRS | | | |
| | LCQKRDGPRATMKIMNYDEFQHCWSKFVYSQRE | | | |
| | LFEPWNNLPKYYILLHIMLGEILRHSMDPPTFTFNF | | | |
| | NNEPWVRGRHETYLCYEVERMHNDTWVLLNQRR | | | |
| | GFLCNQAPHKHGFLEGRHAELCFLDVIPFWKLDL | | | |
| | DQDYRVTCFTSWSPCFSCAQEMAKFISKNKHVSL | | | |
| | CIFTARIYDDQGRCQEGLRTLAEAGAKISIMTYSEF | | | |
| | KHCWDTFVDHQGCPFQPWDGLDEHSQDLSGRL | | | |
| | RAILQNQEN | | | |
| APOBEC | ALLTAETFRLQFNNKRRLRRPYYPRKALLCYQLTP | 228 | | #119140 |
| A3H | QNGSTPTRGYFENKKKCHAEICFINEIKSMGLDET | | | |
| | QCYQVTCYLTWSPCSSCAWELVDFIKAHDHLNLG | | | |
| | IFASRLYYHWCKPQQKGLRLLCGSQVPVEVMGFP | | | |
| | EFADCWENFVDHEKPLSFNPYKMLEELDKNSRAI | | | |
| | KRRLERIKQS | | | |
| RrA3F | KPQIRDHRPNPMEAMYPHIFYFHFENLEKAYGRN | 229 | | #138340 |
| | ETWLCFTVEIIKQYLPVPWKKGVFRNQVDPETHC | | | |
| | HAEKCFLSWFCNNTLSPKKNYQVTWYTSWSPCP | | | |
| | ECAGEVAEFLAEHSNVKLTIYTARLYYFWDTDYQE | | | |
| | GLRSLSEEGASVEIMDYEDFQYCWENFVYDDGEP | | | |
| | FKRWKGLKYNFQSLTRRLREILQ | | | |
| ss-APOBEC- | DPQRLRQWPGPGPASRGGYGQRPRIRNPEEWF | 230 | | #138343 |
| 3b | HELSPRTFSFHFRNLRFASGRNRSYICCQVEGKN | | | |
| | CFFQGIFQNQVPPDPPCHAELCFLSWFQSWGLSP | | | |
| | DEHYYVTWFISWSPCCECAAKVAQFLEENRNVSL | | | |
| | SLSAARLYYFWKSESREGLRRLSDLGAQVGIMSF | | | |
| | QDFQHCWNNFVHNLGMPFQPWKKLHKNYQRLVT | | | |
| | ELKQILREEPATYGSPQAQGKVRIGSTAAGLRHSH | | | |
| | SHTRSEAHLRPNHSSRQHRILNPPREARARTCVL | | | |
| | VDASWICYR | | | |
| AID | DSLLMNRRKFLYQFKNVRWAKGRRETYLCYVVKR | 231 | | #100803 |
| | RDSATSFSLDFGYLRNKNGCHVELLFLRYISDWDL | | | |
| | DPGRCYRVTWFTSWSPCYDCARHVADFLRGNPN | | | |
| | LSLRIFTARLYFCEDRKAEPEGLRRLHRAGVQIAIM | | | |
| | TFKDYFYCWNTFVENHERTFKAWEGLHENSVRLS | | | |
| | RQLRRILLPLYEVDDLRDAFRTLGL | | | |
| AIDmono | DPATFTYQFKNVRWAKGRRETYLCYVVKRRDSAT | 232 | | DOI: |
| | SFSLDFGYLRNKNGCHVELLFLRYISDWDLDPGR | | | 10.1016/j. |
| | CYRVTWFTSWSPCYDCARHVADFLRGNPNLSLRI | | | celrep.2018. |
| | FTARLYFCEDRKAEPEGLRRLAEAGVQIAIMTFKD | | | 09.090 |
| | YFYCWNTFVENHERTFKAWEGLHENSVRLSRQL | | | |
| | RRILQ | | | |
| AID-3c | DPATFTYQFKNVRWAKGRRETYLCYVVKRRDSAT | 233 | | DOI: |
| | SFSLDFGYLRNKNGCHVELLFLRYISDWDLDPGR | | | 10.1016/j. |
| | CYRVTWFTSWSPCYDCARHVADFLRGNPNLSLRI | | | celrep.2018. |
| | FTARLYYFQYPCYQEGLRRLHRAGVQIAIMTFKDY | | | 09.090 |
| | FYCWNTFVENHERTFKAWEGLHENSVRLSRQLR | | | |
| | RILQ | | | |
| AID-3f | DPATFTYQFKNVRWAKGRRETYLCYVVKRRDSAT | 234 | | DOI: |
| | SFSLDFGYLRNKNGCHVELLFLRYISDWDLDPGR | | | 10.1016/j. |
| | CYRVTWFTSWSPCYDCARHVADFLRGNPNLSLRI | | | celrep.2018. |
| | FTARLYYFWDTDYQEGLRRLHRAGVQIAIMTFKDY | | | 09.090 |
| | FYCWNTFVENHERTFKAWEGLHENSVRLSRQLR | | | |
| | RILQ | | | |
| PmCDA1 | TDAEYVRIHEKLDIYTFKKQFFNNKKSVSHRCYVLF | 235 | | #100804 |
| | ELKRRGERRACFWGYAVNKPQSGTERGIHAEIFSI | | | |
| | RKVEEYLRDNPGQFTINWYSSWSPCADCAEKILE | | | |
| | WYNQELRGNGHTLKIWACKLYYEKNARNQIGLWN | | | |
| | LRDNGVGLNVMVSEHYQCCRKIFIQSSHNQLNEN | | | |
| | RWLEKTLKRAEKRRSELSIMIQVKILHTTKSPAV | | | |
| ABE7.10 | SEVEFSHEYWMRHALTLAKRAWDEREVPVGAVL | 236 | TadA + TadA* | #102919 |
| | VHNNRVIGEGWNRPIGRHDPTAHAEIMALRQGGL | | (with | |
| | VMQNYRLIDATLYVTLEPCVMCAGAMIHSRIGRV | | linker) | |
| | VFGARDAKTGAAGSLMDVLHHPGMNHRVEITEGI | | | |
| | LADECAALLSDFFRMRRQEIKAQKKAQSSTDSG | | | |
| | GSSGGSSGSETPGTSESATPESSGGSSGGSSEV | | | |
| | EFSHEYWMRHALTLAKRARDEREVPVGAVLVLNN | | | |
| | RVIGEGWNRAIGLHDPTAHAEIMALRQGGLVMQN | | | |
| | YRLIDATLYVTFEPCVMCAGAMIHSRIGRVVFGVR | | | |
| | NAKTGAAGSLMDVLHYPGMNHRVEITEGILADEC | | | |
| | AALLCYFFRMPRQVFNAQKKAQSSTD | | | |
| ABE8e | SEVEFSHEYWMRHALTLAKRARDEREVPVGAVLV | 237 | | #138489 |
| | LNNRVIGEGWNRAIGLHDPTAHAEIMALRQGGLV | | | |
| | MQNYRLIDATLYVTFEPCVMCAGAMIHSRIGRVVF | | | |
| | GVRNSKRGAAGSLMNVLNYPGMNHRVEITEGILA | | | |
| | DECAALLCDFYRMPRQVFNAQKKAQSSIN | | | |
| miniABE7.10 | SEVEFSHEYWMRHALTLAKRARDEREVPVGAVLV | 238 | | DOI: |
| | LNNRVIGEGWNRAIGLHDPTAHAEIMALRQGGLV | | | 10.1038/ |
| | MQNYRLIDATLYVTFEPCVMCAGAMIHSRIGRVVF | | | s41587- |
| | GVRNAKTGAAGSLMDVLHYPGMNHRVEITEGILA | | | 019-0236-6 |
| | DECAALLCYFFRMPRQVFNAQKKAQSSTD | | | |
| ABE6.3 | SEVEFSHEYWMRHALTLAKRAWDEREVPVGAVL | 239 | TadA + TadA* | #102916 |
| | VHNNRVIGEGWNRPIGRHDPTAHAEIMALRQGGL | | (with | |
| | VMQNYRLIDATLYVTLEPCVMCAGAMIHSRIGRV | | linker) | |
| | VFGARDAKTGAAGSLMDVLHHPGMNHRVEITEGI | | | |
| | LADECAALLSDFFRMRRQEIKAQKKAQSSTDSG | | | |
| | GSSGGSSGSETPGTSESATPESSGGSSGGSSEV | | | |
| | EFSHEYWMRHALTLAKRAWDEREVPVGAVLVLN | | | |
| | NRVIGEGWNRSIGLHDPTAHAEIMALRQGGLVMQ | | | |
| | NYRLIDATLYVTFEPCVMCAGAMIHSRIGRVVFGV | | | |
| | RNAKTGAAGSLMDVLHYPGMNHRVEITEGILADE | | | |
| | CAALLCYFFRMRRQVFNAQKKAQSSTD | | | |
| ABE7.8 | SEVEFSHEYWMRHALTLAKRAWDEREVPVGAVL | 240 | TadA + TadA* | #102917 |
| | VHNNRVIGEGWNRPIGRHDPTAHAEIMALRQGGL | | (with | |
| | VMQNYRLIDATLYVTLEPCVMCAGAMIHSRIGRV | | linker) | |
| | VFGARDAKTGAAGSLMDVLHHPGMNHRVEITEGI | | | |
| | LADECAALLSDFFRMRRQEIKAQKKAQSSTDSG | | | |
| | GSSGGSSGSETPGTSESATPESSGGSSGGSSEV | | | |
| | EFSHEYWMRHALTLAKRALDEREVPVGAVLVLNN | | | |
| | RVIGEGWNRAIGLHDPTAHAEIMALRQGGLVMQN | | | |
| | YRLIDATLYVTFEPCVMCAGAMIHSRIGRVVFGVR | | | |
| | NAKTGAAGSLMDVLHYPGMNHRVEITEGILADEC | | | |
| | NALLCYFFRMRRQVFNAQKKAQSSTD | | | |
| ABE7.9 | SEVEFSHEYWMRHALTLAKRAWDEREVPVGAVL | 241 | TadA + TadA* | #194843 |
| | VHNNRVIGEGWNRPIGRHDPTAHAEIMALRQGGL | | (with | |
| | VMQNYRLIDATLYVTLEPCVMCAGAMIHSRIGRV | | linker) | |
| | VFGARDAKTGAAGSLMDVLHHPGMNHRVEITEGI | | | |
| | LADECAALLSDFFRMRRQEIKAQKKAQSSTDSG | | | |
| | GSSGGSSGSETPGTSESATPESSGGSSGGSSEV | | | |
| | EFSHEYWMRHALTLAKRALDEREVPVGAVLVLNN | | | |
| | RVIGEGWNRAIGLHDPTAHAEIMALRQGGLVMQN | | | |
| | YRLIDATLYVTFEPCVMCAGAMIHSRIGRVVFGVR | | | |
| | NAKTGAAGSLMDVLHYPGMNHRVEITEGILADEC | | | |
| | NALLCYFFRMPRQVFNAQKKAQSSTD | | | |
| ABE8.8-m | SEVEFSHEYWMRHALTLAKRARDEREVPVGAVLV | 242 | ABE8 variant | DOI: |
| | LNNRVIGEGWNRAIGLHDPTAHAEIMALRQGGLV | | | 10.1038/ |
| | MQNYRLIDATLYVTFEPCVMCAGAMIHSRIGRVVF | | | s41587-020- |
| | GVRNAKTGAAGSLMDVLHHPGMNHRVEITEGILA | | | 0491-6 |
| | DECAALLCRFFRMPRRVFNAQKKAQSSTD | | | |
| ABE8.8-d | SEVEFSHEYWMRHALTLAKRAWDEREVPVGAVL | 243 | ABE8 variant | DOI: |
| | VHNNRVIGEGWNRPIGRHDPTAHAEIMALRQGGL | | | 10.1038/ |
| | VMQNYRLIDATLYVTLEPCVMCAGAMIHSRIGRVV | | | s41587-020- |
| | FGARDAKTGAAGSLMDVLHHPGMNHRVEITEGIL | | | 0491-6 |
| | ADECAALLSDFFRMRRQEIKAQKKAQSSTDSGGS | | | |
| | SGGSSGSETPGTSESATPESSGGSSGGSSEVEF | | | |
| | SHEYWMRHALTLAKRARDEREVPVGAVLVLNNRV | | | |
| | IGEGWNRAIGLHDPTAHAEIMALRQGGLVMQNYR | | | |
| | LIDATLYVTFEPCVMCAGAMIHSRIGRVVFGVRNA | | | |
| | KTGAAGSLMDVLHHPGMNHRVEITEGILADECAAL | | | |
| | LCRFFRMPRRVFNAQKKAQSSTD | | | |
| ABE8.13-m | SEVEFSHEYWMRHALTLAKRARDEREVPVGAVLV | 244 | ABE8 variant | DOI: |
| | LNNRVIGEGWNRAIGLHDPTAHAEIMALRQGGLV | | | 10.1038/ |
| | MQNYRLYDATLYVTFEPCVMCAGAMIHSRIGRVV | | | s41587-020- |
| | FGVRNAKTGAAGSLMDVLHHPGMNHRVEITEGIL | | | 0491-6 |
| | ADECAALLCRFFRMPRRVFNAQKKAQSSTD | | | |
| ABE8.13-d | SEVEFSHEYWMRHALTLAKRAWDEREVPVGAVL | 245 | ABE8 variant | DOI: |
| | VHNNRVIGEGWNRPIGRHDPTAHAEIMALRQGGL | | | 10.1038/ |
| | VMQNYRLIDATLYVTLEPCVMCAGAMIHSRIGRVV | | | s41587-020- |
| | FGARDAKTGAAGSLMDVLHHPGMNHRVEITEGIL | | | 0491-6 |
| | ADECAALLSDFFRMRRQEIKAQKKAQSSTDSGGS | | | |
| | SGGSSGSETPGTSESATPESSGGSSGGSSEVEF | | | |
| | SHEYWMRHALTLAKRARDEREVPVGAVLVLNNRV | | | |
| | IGEGWNRAIGLHDPTAHAEIMALRQGGLVMQNYR | | | |
| | LYDATLYVTFEPCVMCAGAMIHSRIGRVVFGVRNA | | | |
| | KTGAAGSLMDVLHHPGMNHRVEITEGILADECAAL | | | |
| | LCRFFRMPRRVFNAQKKAQSSTD | | | |
| ABE8.17-m | SEVEFSHEYWMRHALTLAKRARDEREVPVGAVLV | 246 | ABE8 variant | DOI: |
| | LNNRVIGEGWNRAIGLHDPTAHAEIMALRQGGLV | | | 10.1038/ |
| | MQNYRLIDATLYSTFEPCVMCAGAMIHSRIGRVVF | | | s41587-020- |
| | GVRNAKTGAAGSLMDVLHYPGMNHRVEITEGILA | | | 0491-6 |
| | DECAALLCYFFRMPRRVFNAQKKAQSSTD | | | |
| ABE8.17-d | SEVEFSHEYWMRHALTLAKRAWDEREVPVGAVL | 247 | ABE8 variant | DOI: |
| | VHNNRVIGEGWNRPIGRHDPTAHAEIMALRQGGL | | | 10.1038/ |
| | VMQNYRLIDATLYVTLEPCVMCAGAMIHSRIGRVV | | | s41587-020- |
| | FGARDAKTGAAGSLMDVLHHPGMNHRVEITEGIL | | | 0491-6 |
| | ADECAALLSDFFRMRRQEIKAQKKAQSSTDSGGS | | | |
| | SGGSSGSETPGTSESATPESSGGSSGGSSEVEF | | | |
| | SHEYWMRHALTLAKRARDEREVPVGAVLVLNNRV | | | |
| | IGEGWNRAIGLHDPTAHAEIMALRQGGLVMQNYR | | | |
| | LIDATLYSTFEPCVMCAGAMIHSRIGRVVFGVRNA | | | |
| | KTGAAGSLMDVLHYPGMNHRVEITEGILADECAAL | | | |
| | LCYFFRMPRRVFNAQKKAQSSTD | | | |
| ABE8.20-m | SEVEFSHEYWMRHALTLAKRARDEREVPVGAVLV | 248 | ABE8 variant | DOI: |
| | LNNRVIGEGWNRAIGLHDPTAHAEIMALRQGGLV | | | 10.1038/ |
| | MQNYRLYDATLYSTFEPCVMCAGAMIHSRIGRVV | | | s41587-020- |
| | FGVRNAKTGAAGSLMDVLHHPGMNHRVEITEGIL | | | 0491-6 |
| | ADECAALLCRFFRMPRRVFNAQKKAQSSTD | | | |
| ABE8.20-d | SEVEFSHEYWMRHALTLAKRAWDEREVPVGAVL | 249 | ABE8 variant | DOI: |
| | VHNNRVIGEGWNRPIGRHDPTAHAEIMALRQGGL | | | 10.1038/ |
| | VMQNYRLIDATLYVTLEPCVMCAGAMIHSRIGRVV | | | s41587-020- |
| | FGARDAKTGAAGSLMDVLHHPGMNHRVEITEGIL | | | 0491-6 |
| | ADECAALLSDFFRMRRQEIKAQKKAQSSTDSGGS | | | |
| | SGGSSGSETPGTSESATPESSGGSSGGSSEVEF | | | |
| | SHEYWMRHALTLAKRARDEREVPVGAVLVLNNRV | | | |
| | IGEGWNRAIGLHDPTAHAEIMALRQGGLVMQNYR | | | |
| | LYDATLYSTFEPCVMCAGAMIHSRIGRVVFGVRNA | | | |
| | KTGAAGSLMDVLHHPGMNHRVEITEGILADECAAL | | | |
| | LCRFFRMPRRVFNAQKKAQSSTD | | | |

In some embodiments, a deaminase fusion partner comprises an amino acid sequence having at least 50%, at least 60%, at least 70%, at least 80%, at least 90%, at least 95%, at least 97%, at least 98%, at least 99%, or 100% amino acid sequence identity to an amino acid sequence set forth in Table 2. The amino acid sequences shown in Table 2 are shown without an N-terminal methionine; an N-terminal methionine can be added, for example when the deaminase amino acid sequence is at the N-terminal end of the molecule.

In some embodiments, a fusion protein of the disclosure comprises a deaminase, e.g., as described in Table 2 and a uracil glycosylase inhibitor (UGI) domain (e.g., as described in Wu et al., 2022, Mol. Cell 82 (23): 4487-4502, the contents of which are incorporated herein by reference in their entireties.) An exemplary UGI domain comprises the amino acid sequence

(SEQ ID NO: 250)
TNLSDIIEKETGKQLVIQESILMLPEEVEEVIGNKPESDILVHTAYDE
STDENVMLLTSDAPEYKPWALVIQDSNGENKIKML

Type V Cas proteins of the disclosure in the form of a fusion protein comprising a transcriptional repressor or an effector domain thereof can be used, for example, to silence genes via epigenome editing (see, e.g., Cappelluti et al., 2024 Nature 627:416-423, the contents of which are incorporated herein by reference in their entireties). Exemplary effector domains are described in Table 3.

TABLE 3

| | | SEQ ID |
|---|---|---|
| Name | Amino Acid Sequence | NO |
| KRAB | ALSPQHSAVTQGSIIKNKEGMDAKSLTAWSRTLVTFKDVFVDFTREEWKLLDTAQQI | 251 |
| | VYRNVMLENYKNLVSLGYQLTKPDVILRLEKGEEPWLVEREIHQETHPDSETAFEIKS | |
| | SV | |
| KRAB | SRTLVTFKDVFVDFTREEWKLLDTAQQIVYRNVMLENYKNLVSLGYQLTKPDVILRLE | 252 |
| alternative | KGEEPWLV | |
| cdDNMT3A | GTYGLLRRREDWPSRLQMFFANNHDQEFDPPKVYPPVPAEKRKPIRVLSLFDGIAT | 253 |
| | GLLVLKDLGIQVDRYIASEVCEDSITVGMVRHQGKIMYVGDVRSVTQKHIQEWGPFD | |
| | LVIGGSPCNDLSIVNPARKGLYEGTGRLFFEFYRLLHDARPKEGDDRPFFWLFENVV | |
| | AMGVSDKRDISRFLESNPVMIDAKEVSAAHRARYFWGNLPGMNRPLASTVNDKLEL | |
| | QECLEHGRIAKFSKVRTITTRSNSIKQGKDQHFPVFMNEKEDILWCTEMERVFGFPV | |
| | HYTDVSNMSRLARQRLLGRSWSVPVIRHLFAPLKEYFACV | |
| DNMT3L | AAIPALDPEAEPSMDVILVGSSELSSSVSPGTGRDLIAYEVKANQRNIEDICICCGSLQ | 254 |
| | VHTQHPLFEGGICAPCKDKFLDALFLYDDDGYQSYCSICCSGETLLICGNPDCTRCY | |
| | CFECVDSLVGPGTSGKVHAMSNWVCYLCLPSSRSGLLQRRRKWRSQLKAFYDRE | |
| | SENPLEMFETVPVWRRQPVRVLSLFEDIKKELTSLGFLESGSDPGQLKHVVDVTDT | |
| | VRKDVEEWGPFDLVYGATPPLGHTCDRPPSWYLFQFHRLLQYARPKPGSPRPFFW | |
| | MFVDNLVLNKEDLDVASRFLEMEPVTIPDVHGGSLQNAVRVWSNIPAIRSRHWALVS | |
| | EEELSLLAQNKQSSKLAAKWPTKLVKNCFLPLREYFKYFSTELTSSL | |
| DNMT3A- | NHDQEFDPPKVYPPVPAEKRKPIRVLSLFDGIATGLLVLKDLGIQVDRYIASEVCEDSI | 255 |
| DNMT3L | TVGMVRHQGKIMYVGDVRSVTQKHIQEWGPFDLVIGGSPCNDLSIVNPARKGLYEG | |
| dimer | TGRLFFEFYRLLHDARPKEGDDRPFFWLFENVVAMGVSDKRDISRFLESNPVMIDA | |
| | KEVSAAHRARYFWGNLPGMNRPLASTVNDKLELQECLEHGRIAKFSKVRTITTRSN | |
| | SIKQGKDQHFPVFMNEKEDILWCTEMERVFGFPVHYTDVSNMSRLARQRLLGRSW | |
| | SVPVIRHLFAPLKEYFACVSSGNSNANSRGPSFSSGLVPLSLRGSHMAAIPALDPEA | |
| | EPSMDVILVGSSELSSSVSPGTGRDLIAYEVKANQRNIEDICICCGSLQVHTQHPLFE | |
| | GGICAPCKDKFLDALFLYDDDGYQSYCSICCSGETLLICGNPDCTRCYCFECVDSLV | |
| | GPGTSGKVHAMSNWVCYLCLPSSRSGLLQRRRKWRSQLKAFYDRESENPLEMFE | |
| | TVPVWRRQPVRVLSLFEDIKKELTSLGFLESGSDPGQLKHVVDVTDTVRKDVEEWG | |
| | PFDLVYGATPPLGHTCDRPPSWYLFQFHRLLQYARPKPGSPRPFFWMFVDNLVLNK | |
| | EDLDVASRFLEMEPVTIPDVHGGSLQNAVRVWSNIPAIRSRHWALVSEEELSLLAQN | |
| | KQSSKLAAKWPTKLVKNCFLPLREYFKYFSTELTSSL | |

In some embodiments, an effector domain fusion partner comprises an amino acid sequence having at least 50%, at least 60%, at least 70%, at least 80%, at least 90%, at least 95%, at least 97%, at least 98%, at least 99%, or 100% amino acid sequence identity to an amino acid sequence set forth in Table 3. The amino acid sequences shown in Table 3 are shown without an N-terminal methionine; an N-terminal methionine can be added, for example when the effector domain amino acid sequence is at the N-terminal end of the molecule.

In some embodiments, a fusion protein of the disclosure comprises a means for synthesizing DNA from a single-stranded template, for example a reverse transcriptase, e.g., a MMLV reverse transcriptase (see, WO 2021/226558, the contents of which are incorporated herein by reference in their entireties). An exemplary reverse transcriptase comprises the amino acid sequence

(SEQ ID NO: 256)
TLNIEDEYRLHETSKEPDVSLGSTWLSDFPQAWAETGGMGLAVRQAPLIIPLKATSTPVSIKQYPMSQEA
RLGIKPHIQRLLDQGILVPCQSPWNTPLLPVKKPGTNDYRPVQDLREVNKRVEDIHPTVPNPYNLLSGLPP
SHQWYTVLDLKDAFFCLRLHPTSQPLFAFEWRDPEMGISGQLTWTRLPQGFKNSPTLFNEALHRDLADF
RIQHPDLILLQYVDDLLLAATSELDCQQGTRALLQTLGNLGYRASAKKAQICQKQVKYLGYLLKEGQRWL
TEARKETVMGQPTPKTPRQLREFLGKAGFCRLFIPGFAEMAAPLYPLTKPGTLFNWGPDQQKAYQEIKQ
ALLTAPALGLPDLTKPFELFVDEKQGYAKGVLTQKLGPWRRPVAYLSKKLDPVAAGWPPCLRMVAAIAVLT
KDAGKLTMGQPLVILAPHAVEALVKQPPDRWLSNARMTHYQALLLDTDRVQFGPVVALNPATLLPLPEEG
LQHNCLDILAEAHGTRPDLTDQPLPDADHTWYTDGSSLLQEGQRKAGAAVTTETEVIWAKALPAGTSAQ
RAELIALTQALKMAEGKKLNVYTDSRYAFATAHIHGEIYRRRGWLTSEGKEIKNKDEILALLKALFLPKRLSII
HCPGHQKGHSAEARGNRMADQAARKAAITETPDTSTLLIENSSP (see, Chen et al., 2021,
Cell 184(22): 5635-5652, the contents of which are incorporated herein by
reference in their entireties).

Another exemplary reverse transcriptase comprises the amino acid sequence

(SEQ ID NO: 257)
ISSSKHTLSQMNKVSNIVKEPELPDIYKEFKDITADTNTEKLPKPIKGLEFEVELTQENYRLPIRNYPLTPVK
MQAMNDEINQGLKGGIIRESKAINACPVIFVPRKEGTLRMVVDYRPLNKYVKPNVYPLPLIEQLLAKIQGST
IFTKLDLKSAYHQIRVRKGDEHKLAFRCPRGVFEYLVMPYGIKTAPAHFQYFINTILGEAKESHVVCYMDDI
LIHSKSESEHVKHVKDVLQKLKNANLIINQAKCEFHQSQVKFLGYHISEKGLTPCQENIDKVLQWKQPKNQ
KELRQFLGQVNYLRKFIPKTSQLTHPLNKLLKKDVRWKWTPTQTQAIENIKQCLVSPPVLRHFDFSKKILLE
TDVSDVAVGAVLSQKHDDDKYYPVGYYSAKMSKAQLNYSVSDKEMLAIIKSLEHWRHYLESTIEPFKILTD
HRNLIGRITNESEPENKRLARWQLFLQDFNFEINYRPGSANHIADALSRIVDETEPIPKDNEDNSINFVNQI
SIS (see, Doman et al., 2023, Cell 186(18): 3983-4002, the contents of
which are incorporated herein by reference in their entireties).

In some embodiments, a reverse transcriptase fusion partner comprises an amino acid sequence having at least 50%, at least 60%, at least 70%, at least 80%, at least 90%, at least 95%, at least 97%, at least 98%, at least 99%, or 100% amino acid sequence identity to SEQ ID NO:256 or SEQ ID NO:257.

Type V Cas proteins of the disclosure in the form of a fusion protein comprising a reverse transcriptase (RT) can be used as a prime editor to carry out precise DNA editing without double-stranded DNA breaks.

In some embodiments, a Type V Cas protein described herein can be used for prime editing, e.g., with different Circular RNA-mediated Prime Editors (CPEs) for various editing scenarios: for example a nickase-dependent CPE (niCPE), a nuclease-dependent CPE (nuCPE), a split nickase-dependent CPE (sniCPE), or a split nuclease-dependent CPE (snuCPE) (Liang et al., 2004, Nature Biotechnology doi.org/10.1038/s41587-023-02095-x).

In some embodiments, a fusion protein of the disclosure comprises one or more nuclear localization signals positioned N-terminal and/or C-terminal to a Type V Cas protein sequence (e.g., a Type V Cas protein comprising an amino acid sequence set forth in Section 6.2). In some embodiments, a fusion protein of the disclosure comprises a C-terminal nuclear localization signal, for example having the sequence KRTADGSEFESPKKKRKV (SEQ ID NO: 122). In some embodiments, a fusion protein of the disclosure comprises a N-terminal nuclear localization signal, for example having the sequence KRTADGSEFESPKKKRKV (SEQ ID NO: 122). In some embodiments, a fusion protein of the disclosure comprises a N-terminal and a C-terminal nuclear localization signal, for example each having the sequence KRTADGSEFESPKKKRKV (SEQ ID NO:122).

The disclosure provides chimeric Type V Cas proteins comprising one or more domains of an ZWGD Type V Cas protein and one or more domains of one or more different proteins (e.g., one or more different Type V Cas proteins); a chimeric Type V Cas proteins comprising one or more domains of an ZJHK Type V Cas protein and one or more domains of one or more different proteins (e.g., one or more different Type V Cas proteins); a chimeric Type V Cas proteins comprising one or more domains of an ZIKV Type V Cas protein and one or more domains of one or more different proteins (e.g., one or more different Type V Cas proteins); a chimeric Type V Cas proteins comprising one or more domains of an ZZFT Type V Cas protein and one or more domains of one or more different proteins (e.g., one or more different Type V Cas proteins); a chimeric Type V Cas proteins comprising one or more domains of an YYAN Type V Cas protein and one or more domains of one or more different proteins (e.g., one or more different Type V Cas proteins); a chimeric Type V Cas proteins comprising one or more domains of an ZZGY Type V Cas protein and one or more domains of one or more different proteins (e.g., one or more different Type V Cas proteins); a chimeric Type V Cas proteins comprising one or more domains of an ZKBG Type V Cas protein and one or more domains of one or more different proteins (e.g., one or more different Type V Cas proteins); a chimeric Type V Cas proteins comprising one or more domains of an ZZKD Type V Cas protein and one or more domains of one or more different proteins (e.g., one or more different Type V Cas proteins); a chimeric Type V Cas proteins comprising one or more domains of an ZXPB Type V Cas protein and one or more domains of one or more different proteins (e.g., one or more different Type V Cas proteins); a chimeric Type V Cas proteins comprising one or more domains of an ZPPX Type V Cas protein and one or more domains of one or more different proteins (e.g., one or more different Type V Cas proteins); a chimeric Type V Cas proteins comprising one or more domains of an ZXHQ Type V Cas protein and one or more domains of one or more different proteins (e.g., one or more different Type V Cas proteins); a chimeric Type V Cas proteins comprising one or more domains of an ZQKH Type V Cas protein and one or more domains of one or more different proteins (e.g., one or more different Type V Cas proteins); a chimeric Type V Cas proteins comprising one or more domains of an ZRGM Type V Cas protein and one or more domains of one or more different proteins (e.g., one or more different Type V Cas proteins); a chimeric Type V Cas proteins comprising one or more domains of an ZTAE Type V Cas protein and one or more domains of one or more different proteins (e.g., one or more different Type V Cas proteins); a chimeric Type V Cas proteins comprising one or more domains of an ZSQQ Type V Cas protein and one or more domains of one or more different proteins (e.g., one or more different Type V Cas proteins); a chimeric Type V Cas proteins comprising one or more domains of an ZSYN Type V Cas protein and one or more domains of one or more different proteins (e.g., one or more different Type V Cas proteins); a chimeric Type V Cas proteins comprising one or more domains of an ZRBH Type V Cas protein and one or more domains of one or more different proteins (e.g., one or more different Type V Cas proteins); a chimeric Type V Cas proteins comprising one or more domains of an ZWPU Type V Cas protein and one or more domains of one or more different proteins (e.g., one or more different Type V Cas proteins); a chimeric Type V Cas proteins comprising one or more domains of an ZZQE Type V Cas protein and one or more domains of one or more different proteins (e.g., one or more different Type V Cas proteins); a chimeric Type V Cas proteins comprising one or more domains of an ZRXE Type V Cas protein and one or more domains of one or more different proteins (e.g., one or more different Type V Cas proteins).

The domain structures of the Type V Cas proteins described herein were inferred by multiple alignment with the amino acid sequences of Type V Cas proteins for which the crystal structure is known and for which it is thus possible to define the boundaries of each functional domain. The domains identified in Type V Cas proteins are: wedge (WED) domain (WED-1 domain, WED-II domain, WED-III domain), the RuvC catalytic domain (discontinuous, represented by RuvC-I domain, RuvC-II domain, RuvCIII domain), recognition (REC) domain (REC1 domain, REC2 domain), PAM-interacting domain (PI domain), bridge helix (BH domain), and nuclease (NUC) domain,

Table 4 below report the amino acid positions corresponding to the boundaries between different functional domains in full-length wild-type ZWGD (SEQ ID NO:2), ZJHK (SEQ ID NO:8), ZIKV (SEQ ID NO: 14), ZZFT (SEQ ID NO:20), YYAN (SEQ ID NO:26), ZZGY (SEQ ID NO:32), ZKBG (SEQ ID NO:38), ZZKD (SEQ ID NO:44), ZXPB (SEQ ID NO:50), ZPPX (SEQ ID NO:56), ZXHQ (SEQ ID NO:62), ZQKH (SEQ ID NO: 68), ZRGM (SEQ ID NO:74), ZTAE (SEQ ID NO:80), ZSQQ (SEQ ID NO:86), ZSYN (SEQ ID NO: 92), ZRBH (SEQ ID NO:98), ZWPU (SEQ ID NO: 104), ZZQE (SEQ ID NO: 110), and ZRXE (SEQ ID NO: 116) Type V Cas proteins.

TABLE 4
Amino Acid Positions of Domains of Exemplified Type V Cas Proteins

| Type V Cas | WED-I | REC1 | REC2 | WED-II | PI | WED-III | RuvC-I | BH | RuvC-II | NUC | RuvC-III |
|---|---|---|---|---|---|---|---|---|---|---|---|
| ZRGM | 1- | 25- | 292- | 507- | 575- | 700- | 867- | 927- | 944- | 1054- | 1236- |
| | 24 | 291 | 506 | 574 | 699 | 866 | 926 | 943 | 1053 | 1235 | 1284 |
| ZZGY | 1- | 24- | 308- | 519- | 591- | 711- | 881- | 945- | 962- | 1071- | 1253- |
| | 23 | 307 | 518 | 590 | 710 | 880 | 944 | 961 | 1070 | 1252 | 1302 |
| ZRXE | 1- | 24- | 305- | 546- | 616- | 707- | 839- | 902- | 919- | 1027- | 1203- |
| | 23 | 304 | 545 | 615 | 706 | 838 | 901 | 918 | 1026 | 1202 | 1252 |
| ZRBH | 1- | 24- | 295- | 532- | 603- | 694- | 828- | 887- | 904- | 1012- | 1188- |
| | 23 | 294 | 531 | 602 | 693 | 827 | 886 | 903 | 1011 | 1187 | 1235 |
| ZSYN | 1- | 27- | 341- | 574- | 650- | 741- | 874- | 938- | 955- | 1063- | 1238- |
| | 26 | 340 | 573 | 649 | 740 | 873 | 937 | 954 | 1062 | 1237 | 1283 |
| ZKBG | 1- | 24- | 303- | 531- | 600- | 724- | 858- | 925- | 942- | 1054- | 1233- |
| | 23 | 302 | 530 | 599 | 723 | 857 | 924 | 941 | 1053 | 1232 | 1271 |
| ZXHQ | 1- | 27- | 290- | 525- | 601- | 692- | 812- | 910- | 927- | 1040- | 1210- |
| | 26 | 289 | 524 | 600 | 691 | 811 | 909 | 926 | 1039 | 1209 | 1262 |
| ZZQE | 1- | 26- | 308- | 543- | 613- | 704- | 836- | 899- | 916- | 1024- | 1200- |
| | 25 | 307 | 542 | 612 | 703 | 835 | 898 | 915 | 1023 | 1199 | 1249 |
| YYAN | 1- | 23- | 292- | 518- | 590- | 678- | 815- | 875- | 892- | 998- | 1169- |
| | 22 | 291 | 517 | 589 | 677 | 814 | 874 | 891 | 997 | 1168 | 1215 |
| ZQKH | 1- | 26- | 249- | 444- | 505- | 610- | 721- | 778- | 795- | 905- | 1090- |
| | 25 | 248 | 443 | 504 | 609 | 720 | 777 | 794 | 904 | 1089 | 1133 |
| ZZFT | 1- | 24- | 297- | 525- | 596- | 699- | 830- | 896- | 913- | 1025- | 1202- |
| | 23 | 296 | 524 | 595 | 698 | 829 | 895 | 912 | 1024 | 1201 | 1245 |
| ZIKV | 1- | 24- | 282- | 497- | 565- | 668- | 791- | 846- | 863- | 971- | 1147- |
| | 23 | 281 | 496 | 564 | 667 | 790 | 845 | 862 | 970 | 1146 | 1195 |
| ZWPU | 1- | 27- | 297- | 527- | 597- | 689- | 822- | 885- | 902- | 1010- | 1194- |
| | 26 | 296 | 526 | 596 | 688 | 821 | 884 | 901 | 1009 | 1193 | 1243 |
| ZPPX | 1- | 21- | 300- | 537- | 607- | 720- | 854- | 916- | 933- | 1041- | 1216- |
| | 20 | 299 | 536 | 606 | 719 | 853 | 915 | 932 | 1040 | 1215 | 1264 |
| ZZKD | 1- | 25- | 291- | 514- | 583- | 674- | 805- | 872- | 889- | 997- | 1175- |
| | 24 | 290 | 513 | 582 | 673 | 804 | 871 | 888 | 996 | 1174 | 1220 |
| ZSQQ | 1- | 27- | 310- | 549- | 618- | 721- | 888- | 953- | 970- | 1078- | 1263- |
| | 26 | 309 | 548 | 617 | 720 | 887 | 952 | 969 | 1077 | 1262 | 1310 |
| ZJHK | 1- | 25- | 286- | 516- | 586- | 711- | 877- | 934- | 951- | 1062- | 1243- |
| | 24 | 285 | 515 | 585 | 710 | 876 | 933 | 950 | 1061 | 1242 | 1294 |
| ZWGD | 1- | 31- | 311- | 564- | 639- | 733- | 868- | 937- | 954- | 1061- | 1247- |
| | 30 | 310 | 563 | 638 | 732 | 867 | 936 | 953 | 1060 | 1246 | 1292 |
| ZTAE | 1- | 23- | 323- | 551- | 625- | 716- | 882- | 937- | 954- | 1062- | 1242- |
| | 22 | 322 | 550 | 624 | 715 | 881 | 936 | 953 | 1061 | 1241 | 1289 |
| ZXPB | 1- | 23- | 276- | 505- | 575- | 666- | 798- | 853- | 870- | 978- | 1152- |
| | 22 | 275 | 504 | 574 | 665 | 797 | 852 | 869 | 977 | 1151 | 1201 |

A chimeric Type V Cas protein can comprise one of more of the following domains (e.g., one or more, two or more, three or more, four or more, five or more, six or more, seven or more) from a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, and/or a ZRXE Type V Cas protein, and one or more domains from one or more other proteins, for example Cas12a: WED-1 domain, REC1 domain, REC2 domain, WED-II domain, PI domain, WED-III domain, RuvC-I domain, BH domain, RuvC-II domain, NUC domain, or RuvC-III domain. For example, the PID domain can be swapped between different Type V Cas proteins to change the PAM specificity of the resulting chimeric protein (which is given by the donor PID domain). Swapping of other domains or portions of them is also within the scope of the disclosure (e.g., through protein shuffling).

In some embodiments, a Type V Cas protein of the disclosure comprises one, two, three, four, five, six, seven, or eight of a WED-1 domain, REC1 domain, REC2 domain, WED-II domain, PI domain, WED-III domain, RuvC-I domain, BH domain, RuvC-II domain, NUC domain, or RuvC-III domain arranged in the N-terminal to C-terminal direction. In some embodiments, all domains are from one Type

V Cas protein as described herein, e.g., ZWGD, ZJHK, ZIKV), ZZFT, YYAN, ZZGY, ZKBG, ZZKD, ZXPB, ZPPX, ZXHQ, ZQKH, ZRGM, ZTAE, ZSQQ, ZSYN, ZRBH, ZWPU, ZZQE, or ZRXE. In other embodiments, one or more domains (e.g., one domain), e.g., a PID domain, is from another Type V Cas protein, for example a Cas12a protein from Alicyclobacillus acidoterrestris, Bacillus thermoamylovorans, Lachnospiraceae bacterium (e.g., LbCas12a, NCBI Reference Sequence WP_051666128.1), Acidaminococcus sp. BV3L6 (e.g., AsCas12a, NCBI Reference Sequence WP_021736722.1), Arcobacter butzleri L348 (e.g., AbCas12a, GeneBank ID: JAIQ01000039.1), Agathobacter rectalisstrain 2789STDY5834884 (e.g., ArCas12a, GeneBank ID: CZAJ01000001.1), Bacteroidetes oraltaxon 274 str. F0058 (e.g., BoCas12a, GeneBank ID: NZ_GG774890.1), Butyrivibrio sp. NC3005 (e.g., BsCas12a, GeneBank ID: NZ_AUKC01000013.1), Candidate division WS6 bacterium GW2011_GWA2_37_6 US52_C0007 (e.g., C6Cas12a, GeneBank ID: LBTH01000007.1), Helcococcus kunzii ATCC 51366 (e.g., HkCas12a, GeneBank ID: JH601088.1/AGEI01000022.1), Lachnospira pectinoschiza strain 2789STDY5834836 (e.g., LpCas12a, GeneBank ID: CZAK01000004), Oribacterium sp. NK2B42 (e.g., OsCas12a, GeneBank ID: NZ_KE384190.1), Pseudobutyrivibrio ruminis CF1b (e.g., PrCas12a, GeneBank ID: NZ_KE384121.1), Proteocatella sphenisci DSM 23131 (e.g., PsCas12a, GeneBank ID: NZ_KE384028.1), Pseudobutyrivibrio xylanivoransstrain DSM 10317 (e.g., PxCas12a, GeneBank ID: FMWK01000002.1), Sneathia amniistrain SN35 (e.g., SaCas12a, GeneBank ID: CP011280.1), Francisella novicida, or Leptotrichia shahii. In addition, one or more amino acid substitutions can be introduced in one or more domains to modify the properties of the resulting nuclease in terms of editing activity, targeting specificity or PAM recognition specificity. For example, one or more amino acid substitutions can be introduced to provide nickase activity. Exemplary amino acid substitutions in Cas12a providing nickase activity are the D908, E993, R1226 and D1263. Corresponding substitutions can be introduced into the Type V Cas nucleases of the disclosure to provide nickases and catalytically inactive Cas proteins. Positions corresponding to such Cas12a positions for Type V Cas proteins of the disclosure as shown in Table 5. Nickases and catalytically inactive Type V Cas proteins of the disclosure can be used, for example, in base editors comprising a cytosine or adenosine deaminase fusion partner. Catalytically inactive Type V Cas proteins can also be used, for example, as fusion partners for transcriptional activators or repressors.

TABLE 5

| | | | | | Reference |
|---|---|---|---|---|---|
| | Position | Position | Position | Position | SEQ ID NO |
| | corresponding | corresponding | corresponding | corresponding | defining |
| Type V Cas | to D908 of | to E993 of | to R1226 of | to D1263 of | amino acid |
| Protein | AsCas12a | AsCas12a | AsCas12a | AsCas12a | numbering |
| ZWGD | 891 | 990 | 1200 | 1248 | 2 |
| ZJHK | 900 | 987 | 1203 | 1244 | 8 |
| ZIKV | 814 | 899 | 1111 | 1148 | 14 |
| ZZFT | 856 | 949 | 1166 | 1203 | 20 |
| YYAN | 838 | 928 | 1135 | 1170 | 26 |
| ZZGY | 905 | 998 | 1214 | 1254 | 32 |
| ZKBG | 885 | 978 | 1194 | 1234 | 38 |
| ZZKD | 828 | 925 | 1138 | 1176 | 44 |
| ZXPB | 821 | 906 | 1116 | 1153 | 50 |
| ZPPX | 877 | 969 | 1181 | 1217 | 56 |
| ZXHQ | 836 | 963 | 1172 | 1211 | 62 |
| ZQKH | 744 | 831 | 1048 | 1091 | 68 |
| ZRGM | 890 | 980 | 1194 | 1237 | 74 |
| ZTAE | 905 | 990 | 1206 | 1243 | 80 |
| ZSQQ | 913 | 1006 | 1219 | 1264 | 86 |
| ZSYN | 902 | 991 | 1200 | 1239 | 92 |
| ZRBH | 851 | 940 | 1152 | 1189 | 98 |
| ZWPU | 845 | 938 | 1153 | 1195 | 104 |
| ZZQE | 859 | 952 | 1164 | 1201 | 110 |
| ZRXE | 862 | 955 | 1167 | 1204 | 116 |

### 6.3. Guide RNAs

The disclosure provides crRNA scaffolds and gRNA molecules that can be used with Type V Cas proteins of the disclosure to edit genomic DNA, for example mammalian DNA, e.g., human DNA. gRNAs of the disclosure typically comprise a spacer of 15 to 30 nucleotides in length. The spacer can be positioned 3′ of a crRNA scaffold to form a full gRNA.

An exemplary crRNA scaffold sequence that can be used for ZWGD Type V Cas gRNAs
comprises.
(SEQ ID NO: 144)
ACGAUUAGAAAUAAUUUCUACUGUUGUAGAU
An exemplary crRNA scaffold sequence that can be used for ZJHK Type V Cas gRNAs
comprises.
(SEQ ID NO: 145)
CUUUGAAAGAAUAUAAUUUCUACUGAAAGUGUAGAU
An exemplary crRNA scaffold sequence that can be used for ZIKV Type V Cas gRNAs
comprises .
(SEQ ID NO: 146)
GUUUAAUAAUAAUACAUAAUUUCUACUAUUGUAGAU.
An exemplary crRNA scaffold sequence that can be used for ZZFT Type V Cas gRNAs
comprises.
(SEQ ID NO: 147)
GUCUAUAAGACUAAUUUAAUUUCUACUAUUGUAGAU
An exemplary crRNA scaffold sequence that can be used for YYAN Type V Cas gRNAs
comprises.
(SEQ ID NO: 148)
GUUUAUAAACCUUAUCUAAUUUCUACUGUUGUAGAU
An exemplary crRNA scaffold sequence that can be used for ZZGY Type V Cas gRNAs
comprises.
(SEQ ID NO: 149)
UCUAAAGCUCUUUAAGAAUUUCUACUUUCGUAGAU
An exemplary crRNA scaffold sequence that can be used for ZKBG Type V Cas gRNAs
comprises.
(SEQ ID NO: 150)
CUAAGAGGCUCAAAUAAUUUCUACUAUUGUAGAU
An exemplary crRNA scaffold sequence that can be used for ZZKD Type V Cas gRNAs
comprises.
(SEQ ID NO: 151)
CCUUUGGAAGUACUAAGAAUUUCUACUGUUGUAGAU
An exemplary crRNA scaffold sequence that can be used for ZZKD Type V Cas gRNAs
comprises.
(SEQ ID NO: 211)
GAAUUUCUACUGUUGUAGAU
An exemplary crRNA scaffold sequence that can be used for ZXPB Type V Cas gRNAs
comprises.
(SEQ ID NO: 152)
GGCUAUAAAAGCCAUAUAAUUUCUACUAUUGUAGAU
An exemplary crRNA scaffold sequence that can be used for ZPPX Type V Cas gRNAs
comprises.
(SEQ ID NO: 153)
GACUAUUAAGUCUUUUGAAUUUCUACUGUUGUAGAU
An exemplary crRNA scaffold sequence that can be used for ZXHQ Type V Cas gRNAs
comprises.
(SEQ ID NO: 154)
UCUAGAAUAUAUAGGUAAUUUCUACUUAUGUAGAU
An exemplary crRNA scaffold sequence that can be used for ZQKH Type V Cas gRNAs
comprises.
(SEQ ID NO: 155)
GGCAAUAAGCCAUAUACAAUUUCUACUGUAUGUAGAU
An exemplary crRNA scaffold sequence that can be used for ZRGM Type V Cas gRNAs
comprises.
(SEQ ID NO: 156)
GUCUGAAAGACUAUAUAAUUUCUACUUCGUGUAGAU
An exemplary crRNA scaffold sequence that can be used for ZRGM Type V Cas gRNAs
comprises.
(SEQ ID NO: 213)
AAUUUCUACUUCGUGUAGAU
An exemplary crRNA scaffold sequence that can be used for ZTAE Type V Cas gRNAs
comprises.
(SEQ ID NO: 157)
GUCUACGGAACGUCUGUAAUUUCUACUGUUGUAGAU
An exemplary crRNA scaffold sequence that can be used for ZSQQ Type V Cas gRNAs
comprises.
(SEQ ID NO: 158)
UUUAAACGAACUAUUAAAUUUCUACUGUUGUAGAU
An exemplary crRNA scaffold sequence that can be used for ZSYN Type V Cas gRNAs
comprises.
(SEQ ID NO: 159)
GUUUAAUACUUAUAUAUAAUUUCUACUAUUGUAGAU
An exemplary crRNA scaffold sequence that can be used for ZRBH Type V Cas gRNAs
comprises.
(SEQ ID NO: 160)
AAUAAUAAUCCCUUAUAAUUUCUACUUUUGUAGAU
An exemplary crRNA scaffold sequence that can be used for ZWPU Type V Cas gRNAs
comprises.
(SEQ ID NO: 161)
GUCUAUAAGACGAACUAAAUUUCUACUAUUGUAGAU
An exemplary crRNA scaffold sequence that can be used for ZZQE Type V Cas gRNAs
comprises.
(SEQ ID NO: 162)
GGCUACUAAGCCUUUAUAAUUUCUACUAUUGUAGAU
An exemplary crRNA scaffold sequence that can be used for ZZQE Type V Cas gRNAs
comprises.
(SEQ ID NO: 212)
UAAUUUCUACUAUUGUAGAU
An exemplary crRNA scaffold sequence that can be used for ZRXE Type V Cas gRNAs
comprises.
(SEQ ID NO: 163)
GUCUAUAAAGACGAAUGAAUUUCUACUAUUGUAGAU

Type V Cas gRNAs of the disclosure are generally 40-70 nucleotides long (e.g., 50 to 60 nucleotides long, 55 to 65 nucleotides long, or 55 to 60 nucleotides long), but gRNAs of other lengths are also contemplated. For example, a crRNA scaffold described herein can be trimmed to a shorter length or extended at the 5′ end (e.g., as described in Park et al., 2018, Nature Communications, 9:3313), which can be helpful for enhancing gene editing efficacy. Additionally, gRNAs of the disclosure can optionally be chemically modified, which can be useful, for example, to enhance serum stability of a gRNA (see, e.g., Park et al., 2018, Nature Communications, 9:3313). Chemical modifications are further discussed in Section 6.3.2.

Further optimization of the structure can be obtained by introducing targeted base changes into the stems of the gRNA to increase their stability and folding. Such base changes will preferably correspond to the introduction of G: C couples, which are known to generate the strongest Watson-Crick pairing. For the sake of clarity, these substitutions can consist in the introduction of a G or a C in a specific position of a stem together with a complementary substitution in another position of the gRNA sequence which is predicted to base pair with the former, for example according to available bioinformatic tools for RNA folding such as UNAfold or RNAfold.

Stem-loop trimming can also be exploited to stabilize desired secondary structures by removing portions of the guide RNA producing unwanted secondary structures through annealing with other regions of the RNA molecule

### 6.3.1. Spacers

The spacer sequence is partially or fully complementary to a target sequence found in a genomic DNA sequence, for example a human genomic DNA sequence. For example, a spacer sequence can be partially or fully complementary to a nucleotide sequence in a gene having a disease causing mutation. A spacer that is partially complementary to a target sequence can have, for example, one, two, or three mismatches with the target sequence.

gRNAs of the disclosure can comprise a spacer that is 15 to 30 nucleotides in length (e.g., 15 to 25, 16 to 24, 17 to 23, 18 to 22, 19 to 21, 18 to 30, 20 to 28, 22 to 26, or 23 to 25 nucleotides in length). In some embodiments, a spacer is 15 nucleotides in length. In other embodiments, a spacer is 16 nucleotides in length. In other embodiments, a spacer is 17 nucleotides in length. In other embodiments, a spacer is 18 nucleotides in length. In other embodiments, a spacer is 19 nucleotides in length. In other embodiments, a spacer is 20 nucleotides in length. In other embodiments, a spacer is 21 nucleotides in length. In other embodiments, a spacer is 22 nucleotides in length. In other embodiments, a spacer is 23 nucleotides in length. In other embodiments, a spacer is 24 nucleotides in length. In other embodiments, a spacer is 25 nucleotides in length. In other embodiments, a spacer is 26 nucleotides in length. In other embodiments, a spacer is 27 nucleotides in length. In other embodiments, a spacer is 28 nucleotides in length. In other embodiments, a spacer is 29 nucleotides in length. In other embodiments, a spacer is 30 nucleotides in length.

Type V Cas endonucleases require a specific sequence, called a protospacer adjacent motif (PAM) that is upstream (e.g., directly upstream) of the target sequence on the non-target strand. Thus, spacer sequences for targeting a gene of interest can be identified by scanning the gene for PAM sequences recognized by the Type V Cas protein. Exemplary PAM sequences for Type V Cas proteins of the disclosure are shown in Table 6A-4B. In addition, TTTV is a canonical PAM sequence for Type V-A Cas proteins, and it expected that Type V Cas proteins of the disclosure can recognize the TTTV PAM.

TABLE 6A
Exemplary Type V Cas Protein PAM Sequences
(in silico determined)

| Cas Protein | PAM Sequence |
|---|---|
| ZWGD | TTN |
| ZJHK | TTTN |
| ZIKV | TTTR |
| ZZFT | TTTN, TTTR |
| YYAN | TTTN |
| ZZGY | TTTN, TTTR |
| ZKBG | YTTN |
| ZZKD | TTTN |
| ZXPB | TTTN |
| ZPPX | YTTN, TTN |
| ZZQE | YTTV |

TABLE 6B
Exemplary Type V Cas Protein PAM Sequences
(in vitro determined)

| Cas protein | PAM Sequence |
|---|---|
| ZZKD | NTTV, VTTV, NCTV, TTTT |
| ZRGM | YTTV |
| ZZQE | NYYN, NTTN, NCTV |

Section 7 describes exemplary sequences that can be used to target B2M, TRAC and PD1 genes. Section 7 further describes exemplary sequences that can be used to target AAVS1, BCL11A, EMX1, PCSK9, VEGFA, and Match6 genomic sequences. Exemplary spacer sequences that can be used in gRNAs of the disclosure are set forth in Table 7. In some embodiments, a gRNA of the disclosure comprises a spacer sequence targeting TRAC. In some embodiments, a gRNA of the disclosure comprises a spacer sequence targeting B2M. In some embodiments, a gRNA of the disclosure comprises a spacer sequence targeting PD1. In some embodiments, a gRNA of the disclosure comprises a spacer sequence targeting AAVS1. In some embodiments, a gRNA of the disclosure comprises a spacer sequence targeting BCL11A. In some embodiments, a gRNA of the disclosure comprises a spacer sequence targeting EMX1. In some embodiments, a gRNA of the disclosure comprises a spacer sequence targeting PCSK9. In some embodiments, a gRNA of the disclosure comprises a spacer sequence targeting VEGFA. In some embodiments, a gRNA of the disclosure comprises a spacer sequence targeting Match6.

TABLE 7
Exemplary Spacer Sequences Targeting Endogenous Genomic Loci

| Guide ID | Target | Spacer (5′→3′) | SEQ ID NO. |
|---|---|---|---|
| B2M-g1 | B2M | UGGCCUGGAGGCUAUCCAGCGUG | 164 |
| B2M-g2 | B2M | CUCACGUCAUCCAGCAGAGAAUG | 165 |
| B2M-g3 | B2M | ACUUUCCAUUCUCUGCUGGAUGA | 166 |
| B2M-g4 | B2M | CUGAAUUGCUAUGUGUCUGGGUU | 167 |
| B2M-g5 | B2M | AAUUCUCUCUCCAUUCUUCAGUA | 168 |
| B2M-g8 | B2M | GUGUCAAGCUAUAUCAGGCACCA | 181 |
| B2M-g9 | B2M | AUGUGUCUUUUCCCGAUAUUCCU | 182 |
| B2M-g1_21 nt | B2M | UGGCCUGGAGGCUAUCCAGCG | 183 |
| TRAC-g1 | TRAC | AGAAUCAAAAUCGGUGAAUAGGC | 169 |
| TRAC-g2 | TRAC | UGACACAUUUGUUUGAGAAUCAA | 170 |
| TRAC-g3 | TRAC | GAGUCUCUCAGCUGGUACACGGC | 171 |
| TRAC-g4 | TRAC | UCUGUGAUAUACACAUCAGAAUC | 172 |
| TRAC-g5 | TRAC | AUUCUCAAACAAAUGUGUCACAA | 173 |
| TRAC-g6 | TRAC | UCACUGGAUUUAGAGUCUCUCAG | 184 |
| TRAC-g9 | TRAC | GAUUCUCAAACAAAUGUGUCACA | 185 |
| TRAC-g11 | TRAC | AAGAGGGAAAUGAGAUCAUGUCC | 186 |
| TRAC-g13 | TRAC | ACCGAUUUUGAUUCUCAAACAAA | 187 |
| TRAC-g15 | TRAC | GUCUGUGAUAUACACAUCAGAAU | 188 |
| TRAC g3_20 nt | TRAC | GAGUCUCUCAGCUGGUACAC | 189 |
| TRAC g3_21 nt | TRAC | GAGUCUCUCAGCUGGUACACG | 190 |
| TRAC g3_22 nt | TRAC | GAGUCUCUCAGCUGGUACACGG | 191 |
| TRAC g3_24 nt | TRAC | GAGUCUCUCAGCUGGUACACGGCA | 192 |
| PD1-g1 | PD1 | CCUUCCGCUCACCUCCGCCUGAG | 174 |
| PD1-g2 | PD1 | GCACGAAGCUCUCCGAUGUGUUG | 175 |
| PD1-g3 | PD1 | AUCUGCGCCUUGGGGGCCAGGGA | 176 |
| PD1-g4 | PD1 | GAACUGGCCGGCUGGCCUGGGUG | 177 |
| AAVS1-g1 | AAVS1 | AUUUGGGCAGCUCCCCUACCCCC | 193 |
| AAVS1-g2 | AAVS1 | GGCAGCUCCCCUACCCCCCUUAC | 194 |
| AAVS1-g6 | AAVS1 | CAGGGGUCCGAGAGCUCAGCUAG | 195 |
| AAVS1-g7 | AAVS1 | AUCUGUCCCCUCCACCCCACAGU | 196 |
| EMX1-g2 | EMX1 | UACUUUGUCCUCCGGUUCUGGAA | 197 |
| EMX1-g3 | EMX1 | UCCUCCGGUUCUGGAACCACACC | 198 |
| BCL11A-g1 | BCL11A | AGCCAUCUCACUACAGAUAACUC | 199 |
| BCL11A-g2 | BCL11A | AAGCUAGUCUAGUGCAAGCUAAC | 200 |
| BCL11A-g3 | BCL11A | GCCUCUGAUUAGGGUGGGGGCGU | 201 |
| BCL11A-g4 | BCL11A | UCACAGGCUCCAGGAAGGGUU | 202 |
| PCSK9-g1 | PCSK9 | UCUGCCACCCACCUCCUCACCUU | 203 |
| PCSK9-g2 | PSCK9 | CAGGUCAUCACAGUUGGGGCCAC | 204 |
| VEGFA-g1 | VEGFA | GAGAGUGAGGACGUGUGUGUC | 205 |
| Match6_20 nt | Match6 | GGGUGAUCAGACCCAACAGC | 206 |
| Match6_21 nt | Match6 | GGGUGAUCAGACCCAACAGCA | 207 |
| Match6_22 nt | Match6 | GGGUGAUCAGACCCAACAGCAG | 208 |
| Match6_23 nt | Match6 | GGGUGAUCAGACCCAACAGCAGG | 209 |
| Match6_24 nt | Match6 | GGGUGAUCAGACCCAACAGCAGGU | 210 |

In some embodiments, a gRNA of the disclosure has a spacer whose nucleotide sequence comprises 15 or more consecutive nucleotides from a sequence shown in Table 7. In some embodiments, a gRNA of the disclosure has a spacer whose nucleotide sequence comprises 16 or more consecutive nucleotides from a sequence shown in Table 7. In some embodiments, a gRNA of the disclosure has a spacer whose nucleotide sequence comprises 17 or more consecutive nucleotides from a sequence shown in Table 7. In some embodiments, a gRNA of the disclosure has a spacer whose nucleotide sequence comprises 18 or more consecutive nucleotides from a sequence shown in Table 7. In some embodiments, a gRNA of the disclosure has a spacer whose nucleotide sequence comprises 19 or more consecutive nucleotides from a sequence shown in Table 7. In some embodiments, a gRNA of the disclosure has a spacer whose nucleotide sequence comprises 20 or more consecutive nucleotides from a sequence shown in Table 7. In some embodiments, a gRNA of the disclosure has a spacer whose nucleotide sequence comprises 21 or more consecutive nucleotides from a sequence shown in Table 7. In some embodiments, a gRNA of the disclosure has a spacer whose nucleotide sequence comprises 22 or more consecutive nucleotides from a sequence shown in Table 7. In some embodiments, a gRNA of the disclosure has a spacer whose nucleotide sequence comprises 23 or more consecutive nucleotides from a sequence shown in Table 5. In some embodiments, a gRNA of the disclosure has a spacer whose nucleotide sequence comprises a sequence shown in Table 7.

6.3.2. Modified gRNA Molecules

Guide RNAs can be readily synthesized by chemical means, enabling a number of modifications to be readily incorporated, as described in the art. The disclosed gRNA (e.g., sgRNA) molecules can be unmodified or can contain any one or more of an array of chemical modifications.

While chemical synthetic procedures are continually expanding, purifications of such RNAs by procedures such as high-performance liquid chromatography (HPLC, which avoids the use of gels such as PAGE) tends to become more challenging as polynucleotide lengths increase significantly beyond a hundred or so nucleotides. One approach that can be used for generating chemically modified RNAs of greater length is to produce two or more molecules that are ligated together. Much longer RNAs, such as those encoding a Type V Cas endonuclease, are more readily generated enzymatically. While fewer types of modifications are available for use in enzymatically produced RNAs, there are still modifications that can be used to, for instance, enhance stability, reduce the likelihood or degree of innate immune response, and/or enhance other attributes, as described herein and in the art.

By way of illustration of various types of modifications, especially those used frequently with smaller chemically synthesized RNAs, modifications can comprise one or more nucleotides modified at the 2′ position of the sugar, for instance a 2′-O-alkyl, 2′-O-alkyl-O-alkyl, or 2′-fluoro-modified nucleotide. In some examples, RNA modifications can comprise 2′-fluoro, 2′-amino or 2′-O-methyl modifications on the ribose of pyrimidines, abasic residues, or an inverted base at the 3′ end of the RNA. Such modifications can be routinely incorporated into oligonucleotides and these oligonucleotides have been shown to have a higher Tm (thus, higher target binding affinity) than 2′-deoxyoligonucleotides against a given target.

A number of nucleotide and nucleoside modifications have been shown to make the oligonucleotide into which they are incorporated more resistant to nuclease digestion than the native oligonucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Some oligonucleotides are oligonucleotides with phosphorothioate backbones and those with heteroatom backbones, particularly CH₂—NH—O—CH₂, CH, ˜N(CH₃)—O—CH₂ (known as a methylene (methylimino) or MMI backbone), CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH); amide backbones (see De Mesmaeker et al. 1995, Ace. Chem. Res., 28:366-374); morpholino backbone structures (see U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., 1991, Science 254:1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Morpholino-based oligomeric compounds are described in Braasch and David Corey, 2002, Biochemistry, 41 (14): 4503-4510; Genesis, Volume 30, Issue 3, (2001); Heasman, 2002, Dev. Biol., 243:209-214; Nasevicius et al., 2000, Nat. Genet., 26:216-220; Lacerra et al., 2000, Proc. Natl. Acad. Sci., 97:9591-9596; and U.S. Pat. No. 5,034,506.

Cyclohexenyl nucleic acid oligonucleotide mimetics are described in Wang et al., 2000, J. Am. Chem. Soc., 122:8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts; see U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

One or more substituted sugar moieties can also be included, e.g., one of the following at the 2′ position: OH, SH, SCH₃, F, OCN, OCH₃, OCH₃O(CH₂)n CH₃, O(CH₂)n NH₂, or O(CH₂)n CH₃, where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or bi-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. In some aspects, a modification includes 2′-methoxyethoxy (2′-O—CH₂CH₂OCH₃, also known as 2′-O-(2-methoxyethyl)) (Martin et al., 1995, Helv. Chim. Acta, 78, 486). Other modifications include 2′-methoxy (2′-O—CH₃), 2′-propoxy (2′-OCH₂ CH₂CH₃) and 2′-fluoro (2′-F). Similar modifications can also be made at other positions on the oligonucleotide, particularly the 3′ position of the sugar on the 3′ terminal nucleotide and the 5′ position of 5′ terminal nucleotide. Oligonucleotides can also have sugar mimetics, such as cyclobutyls in place of the pentofuranosyl group.

In some examples, both a sugar and an internucleoside linkage (in the backbone) of the nucleotide units can be replaced with novel groups. The base units can be maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide can be replaced with an amide containing backbone, for example, an aminoethylglycine backbone. The nucleobases can be retained and bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al., 1991, Science, 254:1497-1500.

RNAs such as guide RNAs can also include, additionally or alternatively, nucleobase (often referred to in the art simply as “base”) modifications or substitutions. As used herein, “unmodified” or “natural” nucleobases include adenine (A), guanine (G), thymine (T), cytosine (C), and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxy cytosine and often referred to in the art as 5-Me-C), 5-hydroxymethylcytosine (HMC), glycosyl HMC and gentobiosyl HMC, as well as synthetic nucleobases, e.g., 2-aminoadenine, 2-(methylamino) adenine, 2-(imidazolylalkyl) adenine, 2-(aminoalklyamino) adenine or other heterosub stituted alkyladenines, 2-thiouracil, 2-thiothymine, 5-bromouracil, 5-hydroxymethyluracil, 8-azaguanine, 7-deazaguanine, N6 (6-aminohexyl) adenine, and 2,6-diaminopurine. Komberg, A., DNA Replication, W. H. Freeman & Co., San Francisco, pp. 75-77 (1980); Gebeyehu et al., Nucl. Acids Res. 15:4513 (1997). A “universal” base known in the art, e.g., inosine, can also be included. 5-Me-C substitutions have been shown to increase nucleic acid duplex stability by about 0.6-1.2° C. (Sanghvi, Y. S., in Crooke, S. T. and Lebleu, B., eds., Antisense Research and Applications, CRC Press, Boca Raton, 1993, pp. 276-278) and are aspects of base substitutions.

Modified nucleobases can comprise other synthetic and natural nucleobases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine, and 3-deazaguanine and 3-deazaadenine.

Further, nucleobases can comprise those disclosed in U.S. Pat. No. 3,687,808, those disclosed in ‘The Concise Encyclopedia of Polymer Science and Engineering’, 858-859, Kroschwitz, J. I., ed. John Wiley & Sons, 1990, those disclosed by Englisch et al., Angewandle Chemie, International Edition’, 1991, 30, p. 613, and those disclosed by Sanghvi, Y. S., Chapter 15, Antisense Research and Applications’, 289-302, Crooke, S. T. and Lebleu, B. ea., CRC Press, 1993. Certain of these nucleobases can be useful for increasing the binding affinity of the oligomeric compounds of the invention. These include 5-substituted pyrimidines, 6-azapyrimidines and N-2, N-6 and O-6 substituted purines, comprising 2-aminopropyladenine, 5-propynyluracil and 5-propynylcytosine. 5-methylcytosine substitutions have been shown to increase nucleic acid duplex stability by about 0.6-1.2° C. (Sanghvi, Y. S., Crooke, S. T. and Lebleu, B., eds, ‘Antisense Research and Applications’, CRC Press, Boca Raton, 1993, 276-278) and are aspects of base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,596,091; 5,614,617; 5,681,941; 5,750,692; 5,763,588; 5,830,653; 6,005,096; and U.S. Patent Application Publication 2003/0158403.

Thus, a modified gRNA can include, for example, one or more non-natural sugars, internucleotide linkages and/or bases. It is not necessary for all positions in a given gRNA to be uniformly modified, and in fact more than one of the aforementioned modifications can be incorporated in a single oligonucleotide, or even in a single nucleoside within an oligonucleotide.

The guide RNAs and/or mRNA (or DNA) encoding an endonuclease can be chemically linked to one or more moieties or conjugates that enhance the activity, cellular distribution, or cellular uptake of the oligonucleotide. Such moieties comprise, but are not limited to, lipid moieties such as a cholesterol moiety (Letsinger et al. 1989, Proc. Natl. Acad. Sci. USA, 86:6553-6556); cholic acid (Manoharan et al, 1994, Bioorg. Med. Chem. Let., 4:1053-1060); a thioether, e.g., hexyl-S-tritylthiol (Manoharan et al, 1992, Ann. N. Y. Acad. Sci., 660:306-309; Manoharan et al., 1993, Bioorg. Med. Chem. Let., 3:2765-2770); a thiocholesterol (Oberhauser et al., 1992, Nucl. Acids Res., 20:533-538); an aliphatic chain, e.g., dodecandiol or undecyl residues (Kabanov et al, 1990, FEBS Lett., 259:327-330; Svinarchuk et al, 1993, Biochimie, 75:49-54); a phospholipid, e.g., di-hexadecyl-rac-glycerol or triethylammonium 1,2-di-O-hexadecyl-rac-glycero-3-H-phosphonate (Manoharan et al., 1995, Tetrahedron Lett., 36:3651-3654; and Shea et al, 1990, Nucl. Acids Res., 18:3777-3783); a polyamine or a polyethylene glycol chain (Mancharan et al, 1995, Nucleosides & Nucleotides, 14:969-973); adamantane acetic acid (Manoharan et al, 1995, Tetrahedron Lett., 36:3651-3654); a palmityl moiety (Mishra et al., 1995, Biochim. Biophys. Acta, 1264:229-237); or an octadecylamine or hexylamino-carbonyl-t oxycholesterol moiety (Crooke et al, 1996, J. Pharmacol. Exp. Ther., 277:923-937). See also U.S. Pat. Nos. 4,828,979; 4,948,882; 5,218,105; 5,525,465; 5,541,313; 5,545,730; 5,552,538; 5,578,717; 5,580,731; 5,580,731; 5,591,584; 5, 109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203; 5,451,463; 5,510,475; 5,512,667; 5,514,785; 5,565,552; 5,567,810; 5,574,142; 5,585,481; 5,587,371; 5,595,726; 5,597,696; 5,599,923; 5,599, 928 and 5,688,941.

Sugars and other moieties can be used to target proteins and complexes comprising nucleotides, such as cationic polysomes and liposomes, to particular sites. For example, hepatic cell directed transfer can be mediated via asialoglycoprotein receptors (ASGPRs); see, e.g., Hu, et al., 2014, Protein Pept Lett. 21 (10): 1025-30. Other systems known in the art and regularly developed can be used to target biomolecules of use in the present case and/or complexes thereof to particular target cells of interest.

Targeting moieties or conjugates can include conjugate groups covalently bound to functional groups, such as primary or secondary hydroxyl groups. Conjugate groups of the present disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this present disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism or excretion of the compounds of the present disclosure. Representative conjugate groups are disclosed in International Patent Application Publication WO1993007883, and U.S. Pat. No. 6,287,860. Conjugate moieties include, but are not limited to, lipid moieties such as a cholesterol moiety, cholic acid, a thioether, e.g., hexyl-5-trityl thiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218, 105; 5,525,465; 5,541,313; 5,545,730; 5,552,538; 5,578,717, 5,580,731; 5,580,731; 5,591,584; 5,109, 124; 5,118,802; 5, 138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214, 136; 5,082,830; 5,112,963; 5,214,136; 5,245,022; 5,254,469; 5,258,506; 5,262,536; 5,272,250; 5,292,873; 5,317,098; 5,371,241; 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

A large variety of modifications have been developed and applied to enhance RNA stability, reduce innate immune responses, and/or achieve other benefits that can be useful in connection with the introduction of polynucleotides into human cells, as described herein; see, e.g., the reviews by Whitehead K A et al., 2011, Annual Review of Chemical and Biomolecular Engineering, 2:77-96; Gaglione and Messere, 2010, Mini Rev Med Chem, 10 (7): 578-95; Chernolovskaya et al, 2010, Curr Opin Mol Ther., 12 (2): 158-67; Deleavey et al., 2009, Curr Protoc Nucleic Acid Chem Chapter 16: Unit 16.3; Behlke, 2008, Oligonucleotides 18 (4): 305-19; Fucini et al, 2012, Nucleic Acid Ther 22 (3): 205-210; Bremsen et al, 2012, Front Genet 3:154.

### 6.4. Systems

The disclosure provides systems comprising a Type V Cas protein of the disclosure (e.g., as described in Section 6.2) and a means for targeting the Type V Cas protein to a target genomic sequence. The means for targeting the Type V Cas protein to a target genomic sequence can be a guide RNA (gRNA) (e.g., as described in Section 6.3).

The disclosure also provides systems comprising a Type V Cas protein of the disclosure (e.g., as described in Section 6.2) and a gRNA (e.g., as described in Section 6.3). The systems can comprise a ribonucleoprotein particle (RNP) in which a Type V Cas protein is complexed with a gRNA. Systems of the disclosure can in some embodiments further comprise genomic DNA complexed with the Type V Cas protein and the gRNA. Accordingly, the disclosure provides systems comprising a Type V Cas protein, a genomic DNA, and gRNA, all complexed with one another.

The systems of the disclosure can exist within a cell (whether the cell is in vivo, ex vivo, or in vitro) or outside a cell (e.g., in a particle our outside of a particle).

### 6.5. Nucleic Acids

The disclosure provides nucleic acids (e.g., DNA or RNA) encoding Type V Cas proteins (e.g., a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, or a ZRXE Type V Cas protein), nucleic acids encoding gRNAs of the disclosure (e.g., a single gRNA or combination of gRNAs), nucleic acids encoding both Type V Cas proteins and gRNAs, and pluralities of nucleic acids, for example comprising a nucleic acid encoding a Type V Cas protein and a gRNA.

A nucleic acid encoding a Type V Cas protein and/or gRNA can be, for example, a plasmid or a viral genome (e.g., a lentivirus, retrovirus, adenovirus, or adeno-associated virus genome). Plasmids can be, for example, plasmids for producing virus particles, e.g., lentivirus particles, or plasmids for propagating the Type V Cas and gRNA coding sequences in bacterial (e.g., E. coli) or eukaryotic (e.g., yeast) cells.

A nucleic acid encoding a Type V Cas protein can, in some embodiments, further encode a gRNA. Alternatively, a gRNA can be encoded by a separate nucleic acid (e.g., DNA or mRNA).

Nucleic acids encoding a Type V Cas protein can be codon optimized, e.g., where at least one non-common codon or less-common codon has been replaced by a codon that is common in a host cell. For example, a codon optimized nucleic acid can direct the synthesis of an optimized messenger mRNA, e.g., optimized for expression in a mammalian expression system. As an example, if the intended target nucleic acid is within a human cell, a human codon-optimized polynucleotide encoding Type V Cas can be used for producing a Type V Cas polypeptide. Exemplary codon-optimized sequences are shown in Tables 1A to 1T.

Nucleic acids of the disclosure, e.g., plasmids and viral vectors, can comprise one or more regulatory elements such as promoters, enhancers, and other expression control elements (e.g., transcription termination signals, such as polyadenylation signals and poly-U sequences). Such regulatory elements are described, for example, in Goeddel, 1990, GENE EXPRESSION TECHNOLOGY: METHODS IN ENZYMOLOGY 185, Academic Press, San Diego, Calif. Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest or in particular cell types. Regulatory elements may also direct expression in a temporal-dependent manner, such as in a cell-cycle dependent or developmental stage-dependent manner, which may or may not also be tissue or cell-type specific. In some embodiments, a nucleic acid of the disclosure comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof, e.g., to express a Type V Cas protein and a gRNA separately. Examples of pol III promoters include, but are not limited to, U6 and H1 promoters. Examples of pol II promoters include, but are not limited to, the retroviral Rous Sarcoma virus (RSV) LTR promoter (optionally with the RSV enhancer), the cytomegalovirus (CMV) promoter (optionally with the CMV enhancer) (see, e.g., Boshart et al, 1985, Cell 41:521-530), the SV40 promoter, the dihydrofolate reductase promoter, the β-actin promoter, the phosphoglycerol kinase (PGK) promoter, and EF1α promoters (for example, full length EF1α promoter and the EFS promoter, which is a short, intron-less form of the full EF1α promoter). Exemplary enhancer elements include WPRE; CMV enhancers; the R-U5′ segment in LTR of HTLV-I; SV40 enhancer; and the intron sequence between exons 2 and 3 of rabbit β-globin. It will be appreciated by those skilled in the art that the design of an expression vector can depend on such factors as the choice of the host cell, the level of expression desired, etc.

The term “vector” refers to a polynucleotide molecule capable of transporting another nucleic acid to which it has been linked. One type of polynucleotide vector includes a “plasmid”, which refers to a circular double-stranded DNA loop into which additional nucleic acid segments are or can be ligated. Another type of polynucleotide vector is a viral vector; wherein additional nucleic acid segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some examples, vectors can be capable of directing the expression of nucleic acids to which they are operably linked. Such vectors can be referred to herein as “recombinant expression vectors”, or more simply “expression vectors”, which serve equivalent functions.

The term “operably linked” means that the nucleotide sequence of interest is linked to regulatory sequence(s) in a manner that allows for expression of the nucleotide sequence. The term “regulatory sequence” is intended to include, for example, promoters, enhancers and other expression control elements (e.g., polyadenylation signals). Such regulatory sequences are well known in the art and are described, for example, in Goeddel; Gene Expression Technology: Methods in Enzymology 185, Academic Press, San Diego, CA (1990). Regulatory sequences include those that direct constitutive expression of a nucleotide sequence in many types of host cells, and those that direct expression of the nucleotide sequence only in certain host cells (e.g., tissue-specific regulatory sequences). It will be appreciated by those skilled in the art that the design of the expression vector can depend on such factors as the choice of the target cell, the level of expression desired, and the like.

Vectors can include, but are not limited to, viral vectors based on vaccinia virus, poliovirus, adenovirus, adeno-associated virus (e.g., AAV2, AAV5, AAV7m8, AAV8, AAV9, AAVrh8r, AAVrh10), SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXTI, pSG5, pSVK3, pBPV, pMSG, and pSVLSV40 (Pharmacia). Additional vectors contemplated for eukaryotic target cells include, but are not limited to, the vectors pCTx-I, pCTx-2, and pCTx-3. Other vectors can be used so long as they are compatible with the host cell.

In some examples, a vector can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. The vector can be a self-inactivating vector that either inactivates the viral sequences or the components of the CRISPR machinery or other elements.

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-I promoters (for example, the full EF1α promoter and the EFS promoter), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-l.

An expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, for example a human RHO promoter or human rhodopsin kinase promoter (hGRK), a cell type specific promoter, etc.).

### 6.6. Particles and Cells

The disclosure further provides particles comprising a Type V Cas protein of the disclosure (e.g., a ZWGD Type V Cas protein, a ZJHK Type V Cas protein, a ZIKV Type V Cas protein, a ZZFT Type V Cas protein, a YYAN Type V Cas protein, a ZZGY Type V Cas protein, a ZKBG Type V Cas protein, a ZZKD Type V Cas protein, a ZXPB Type V Cas protein, a ZPPX Type V Cas protein, a ZXHQ Type V Cas protein, a ZQKH Type V Cas protein, a ZRGM Type V Cas protein, a ZTAE Type V Cas protein, a ZSQQ Type V Cas protein, a ZSYN Type V Cas protein, a ZRBH Type V Cas protein, a ZWPU Type V Cas protein, a ZZQE Type V Cas protein, or a ZRXE Type V Cas protein), particles comprising a gRNA of the disclosure, particles comprising a system of the disclosure, and particles comprising a nucleic acid or plurality of nucleic acids of the disclosure. The particles can in some embodiments comprise or further comprise a gRNA, or a nucleic acid encoding the gRNA (e.g., DNA or mRNA). For example, the particles can comprise a RNP of the disclosure. Exemplary particles include lipid nanoparticles, vesicles, viral-like particles (VLPs) and gold nanoparticles. See, e.g., WO 2020/012335, the contents of which are incorporated herein by reference in their entireties, which describes vesicles that can be used to deliver gRNA molecules and Type V Cas proteins to cells (e.g., complexed together as a RNP).

The disclosure provides particles (e.g., virus particles) comprising a nucleic acid encoding a Type V Cas protein of the disclosure. The particles can further comprise a nucleic acid encoding a gRNA. Alternatively, a nucleic acid encoding a Type V Cas protein can further encode a gRNA.

The disclosure further provides pluralities of particles (e.g., pluralities of virus particles). Such pluralities can include a particle encoding a Type V Cas protein and a different particle encoding a gRNA. For example, a plurality of particles can comprise a virus particle (e.g., an AAV2, AAV5, AAV7m8, AAV8, AAV9, AAVrh8r, or AAVrh10 virus particle) encoding a Type V Cas protein and a second virus particle (e.g., an AAV2, AAV5, AAV7m8, AAV8, AAV9, AAVrh8r, or AAVrh10 virus particle) encoding a gRNA. Alternatively, a plurality of particles can comprise a plurality of virus particles where each particle encodes a Type V Cas protein and a gRNA.

The disclosure further provides cells and populations of cells (e.g., ex vivo cells and populations of cells) that can comprise a Type V Cas protein (e.g., introduced to the cell as a RNP) or a nucleic acid encoding the Type V Cas protein (e.g., DNA or mRNA) (optionally also encoding a gRNA). The disclosure further provides cells and populations of cells comprising a gRNA of the disclosure (optionally complexed with a Type V Cas protein) or a nucleic acid encoding the gRNA (e.g., DNA or mRNA) (optionally also encoding a Type V Cas protein). The cells and populations of cells can be, for example, human cells such as a stem cell, e.g., a hematopoietic stem cell (HSC), a pluripotent stem cell, an induced pluripotent stem cell (iPS), or an embryonic stem cell. In some embodiments, the cells and populations of cells are T cells. Methods for introducing proteins and nucleic acids to cells are known in the art. For example, a RNP can be produced by mixing a Type V Cas protein and one or more guide RNAs in an appropriate buffer. An RNP can be introduced to a cell, for example, via electroporation and other methods known in the art.

The cell populations of the disclosure can be cells in which gene editing by the systems of the disclosure has taken place, or cells in which the components of a system of the disclosure have been introduced or expressed but gene editing has not taken place, or a combination thereof. A cell population can comprise, for example, a population in which at least 1%, at least 5%, at least 10%, at least 15%, at least 20%, at least 30%, at least 40%, at least 50%, at least 60%, or at least 70% of the cells have undergone gene editing by a system of the disclosure.

### 6.7. Pharmaceutical Compositions

Also disclosed herein are pharmaceutical formulations and medicaments comprising a Type V Cas protein, gRNA, nucleic acid or plurality of nucleic acids, system, particle, or plurality of particles of the disclosure together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, antinfective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS), and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a Type V Cas protein in a pharmaceutically effective amount sufficient to edit a gene in a cell. The pharmaceutical compositions can be formulated for medical and/or veterinary use.

### 6.8. Methods of Altering a Cell

The disclosure further provides methods of using the Type V Cas proteins, gRNAs, nucleic acids (including pluralities of nucleic acids), systems, and particles (including pluralities of particles) of the disclosure for altering cells.

In one aspect, a method of altering a cell comprises contacting a eukaryotic cell (e.g., a human cell) with a nucleic acid, particle, system or pharmaceutical composition described herein.

Contacting a cell with a disclosed nucleic acid, particle, system or pharmaceutical composition can be achieved by any method known in the art and can be performed in vivo, ex vivo, or in vitro. In some embodiments, the methods can include obtaining one or more cells from a subject prior to contacting the cell(s) with a herein disclosed nucleic acid, particle, system or pharmaceutical composition. In some embodiments, the methods can further comprise returning or implanting the contacted cell or a progeny thereof to the subject.

Type V Cas and gRNA, as well as nucleic acids encoding Type V Cas and gRNAs can be delivered to a cell by any means known in the art, for example, by viral or non-viral delivery vehicles, electroporation or lipid nanoparticles.

A polynucleotide encoding Type V Cas and a gRNA, can be delivered to a cell (ex vivo or in vivo) by a lipid nanoparticle (LNP). LNPs can have, for example, a diameter of less than 1000 nm, 500 nm, 250 nm, 200 nm, 150 nm, 100 nm, 75 nm, 50 nm, or 25 nm. Alternatively, a nanoparticle can range in size from 1-1000 nm, 1-500 nm, 1-250 nm, 25-200 nm, 25-100 nm, 35-75 nm, or 25-60 nm. LNPs can be made from cationic, anionic, neutral lipids, and combinations thereof. Neutral lipids, such as the fusogenic phospholipid DOPE or the membrane component cholesterol, can be included in LNPs as ‘helper lipids’ to enhance transfection activity and nanoparticle stability.

LNPs can also be comprised of hydrophobic lipids, hydrophilic lipids, or both hydrophobic and hydrophilic lipids. Lipids and combinations of lipids that are known in the art can be used to produce a LNP. Examples of lipids used to produce LNPs are: DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Examples of cationic lipids are: 98N12-5, C12-200, DLin-KC2-DMA (KC2), DLin-MC3-DMA (MC3), XTC, MD1, and 7C1. Examples of neutral lipids are: DPSC, DPPC, POPC, DOPE, and SM. Examples of PEG-modified lipids are: PEG-DMG, PEG-CerCI4, and PEG-CerC20. Lipids can be combined in any number of molar ratios to produce a LNP. In addition, the polynucleotide(s) can be combined with lipid(s) in a wide range of molar ratios to produce a LNP.

Type V Cas and/or gRNAs can be delivered to a cell via an adeno-associated viral vector (e.g., of an AAV2, AAV5, AAV7m8, AAV8, AAV9, AAVrh8r, or AAVrh10 serotype), or by another viral vector. Other viral vectors include, but are not limited to lentivirus, adenovirus, alphavirus, enterovirus, pestivirus, baculovirus, herpesvirus, Epstein Barr virus, papovavirus, poxvirus, vaccinia virus, and herpes simplex virus. In some embodiments, a Type V Cas mRNA is formulated in a lipid nanoparticle, while a sgRNA is delivered to a cell in an AAV or other viral vector. In some embodiments, one or more AAV vectors (e.g., one or more AAV2, AAV5, AAV7m8, AAV8, AAV9, AAVrh8r, or AAVrh10 serotype) are used to deliver both a sgRNA and a Type V Cas. In some embodiments, a Type V Cas and a sgRNA are delivered using separate vectors. In other embodiments, a Type V Cas and a sgRNA are delivered using a single vector. BNK Type V Cas and AIK Type V Cas, with their relatively small size, can be delivered with a gRNA (e.g., sgRNA) using a single AAV vector.

Compositions and methods for delivering Type V Cas and gRNAs to a cell and/or subject are further described in PCT Patent Application Publications WO 2019/102381, WO 2020/012335, and WO 2020/053224, each of which is incorporated by reference herein in its entirety.

DNA cleavage can result in a single-strand break (SSB) or double-strand break (DSB) at particular locations within the DNA molecule. Such breaks can be and regularly are repaired by natural, endogenous cellular processes, such as homology-dependent repair (HDR) and non-homologous end-joining (NHEJ). These repair processes can edit the targeted polynucleotide by introducing a mutation, thereby resulting in a polynucleotide having a sequence which differs from the polynucleotide's sequence prior to cleavage by a Type V Cas.

NHEJ and HDR DNA repair processes consist of a family of alternative pathways. Non-homologous end-joining (NHEJ) refers to the natural, cellular process in which a double-stranded DNA-break is repaired by the direct joining of two non-homologous DNA segments. See, e.g. Cahill et al., 2006, Front. Biosci. 11:1958-1976. DNA repair by non-homologous end-joining is error-prone and frequently results in the untemplated addition or deletion of DNA sequences at the site of repair. Thus, NHEJ repair mechanisms can introduce mutations into the coding sequence which can disrupt gene function. NHEJ directly joins the DNA ends resulting from a double-strand break, sometimes with a modification of the polynucleotide sequence such as a loss of or addition of nucleotides in the polynucleotide sequence. The modification of the polynucleotide sequence can disrupt (or perhaps enhance) gene expression.

Homology-dependent repair (HDR) utilizes a homologous sequence, or donor sequence, as a template for inserting a defined DNA sequence at the break point. The homologous sequence can be in the endogenous genome, such as a sister chromatid. Alternatively, the donor can be an exogenous nucleic acid, such as a plasmid, a single-strand oligonucleotide, a double-stranded oligonucleotide, a duplex oligonucleotide or a virus, that has regions of high homology with the nuclease-cleaved locus, but which can also contain additional sequence or sequence changes including deletions that can be incorporated into the cleaved target locus.

A third repair mechanism includes microhomology-mediated end joining (MMEJ), also referred to as “Alternative NHEJ (ANHEJ)”, in which the genetic outcome is similar to NHEJ in that small deletions and insertions can occur at the cleavage site. MMEJ can make use of homologous sequences of a few base pairs flanking the DNA break site to drive a more favored DNA end joining repair outcome. In some instances, it may be possible to predict likely repair outcomes based on analysis of potential microhomologies at the site of the DNA break.

Modifications of a cleaved polynucleotide by HDR, NHEJ, and/or ANHEJ can result in, for example, mutations, deletions, alterations, integrations, gene correction, gene replacement, gene tagging, transgene insertion, nucleotide deletion, gene disruption, translocations and/or gene mutation. The aforementioned process outcomes are examples of editing a polynucleotide.

When performing prime editing, e.g., with a prime editor comprising a Type V Cas protein of the disclosure that comprises a reverse transcriptase, a DNA mismatch repair (MMR) inhibitor can be used in conjunction with the prime editor. Use of MMR inhibitors have been reported to enhance efficiency of prime editing (see, e.g., Chen et al., 2021 Cell 184 (22): 5635-5652, the contents of which are incorporated herein by reference in their entireties). An exemplary MMR inhibitor is MLH1dn, having the amino acid sequence

(SEQ ID NO: 258)
SFVAGVIRRLDETVVNRIAAGEVIQRPANAIKEMIENCLDAKSTSIQVIVKEGGLKLIQIQDNGTGIRKEDLDI
VCERFTTSKLQSFEDLASISTYGFRGEALASISHVAHVTITTKTADGKCAYRASYSDGKLKAPPKPCAGNQ
GTQITVEDLFYNIATRRKALKNPSEEYGKILEVVGRYSVHNAGISFSVKKQGETVADVRTLPNASTVDNIRS
IFGNAVSRELIEIGCEDKTLAFKMNGYISNANYSVKKCIFLLFINHRLVESTSLRKAIETVYAAYLPKNTHPFL
YLSLEISPQNVDVNVHPTKHEVHFLHEESILERVQQHIESKLLGSNSSRMYFTQTLLPGLAGPSGEMVKS
TTSLTSSSTSGSSDKVYAHQMVRTDSREQKLDAFLQPLSKPLSSQPQAIVTEDKTDISSGRARQQDEEML
ELPAPAEVAAKNQSLEGDTTKGTSEMSEKRGPTSSNPRKRHREDSDVEMVEDDSRKEMTAACTPRRRII
NLTSVLSLQEEINEQGHEVLREMLHNHSFVGCVNPQWALAQHQTKLYLLNTTKLSEELFYQILIYDFANFG
VLRLSEPAPLFDLAMLALDSPESGWTEEDGPKEGLAEYIVEFLKKKAEMLADYFSLEIDEEGNLIGLPLLID
NYVPPLEGLPIFILRLATEVNWDEEKECFESLSKECAMFYSIRKQYISEESTLSGQQSEVPGSIPNSWKWT
VEHIVYKALRSHILPPKHFTEDGNILQLANLPDLYKVF.

In some embodiments, an MMR inhibitor is provided in trans with a prime editor.

Advantages of ex vivo cell therapy approaches include the ability to conduct a comprehensive analysis of the therapeutic prior to administration. Nuclease-based therapeutics can have some level of off-target effects. Performing gene correction ex vivo allows a method user to characterize the corrected cell population prior to implantation, including identifying any undesirable off-target effects. Where undesirable effects are observed, a method user may opt not to implant the cells or cell progeny, may further edit the cells, or may select new cells for editing and analysis. Other advantages include ease of genetic correction in iPSCs compared to other primary cell sources. iPSCs are prolific, making it easy to obtain the large number of cells that will be required for a cell-based therapy. Furthermore, iPSCs are an ideal cell type for performing clonal isolations. This allows screening for the correct genomic correction, without risking a decrease in viability.

Although certain cells present an attractive target for ex vivo treatment and therapy, increased efficacy in delivery may permit direct in vivo delivery to such cells. Ideally the targeting and editing is directed to the relevant cells. Cleavage in other cells can also be prevented by the use of promoters only active in certain cell types and/or developmental stages.

Additional promoters are inducible, and therefore can be temporally controlled if the nuclease is delivered as a plasmid. The amount of time that delivered protein and RNA remain in the cell can also be adjusted using treatments or domains added to change the half-life. In vivo treatment would eliminate a number of treatment steps, but a lower rate of delivery can require higher rates of editing. In vivo treatment can eliminate problems and losses from ex vivo treatment and engraftment.

An advantage of in vivo gene therapy can be the ease of therapeutic production and administration. The same therapeutic approach and therapy has the potential to be used to treat more than one patient, for example a number of patients who share the same or similar genotype or allele. In contrast, ex vivo cell therapy typically requires using a subject's own cells, which are isolated, manipulated and returned to the same patient.

Progenitor cells (also referred to as stem cells herein) are capable of both proliferation and giving rise to more progenitor cells, which in turn have the ability to generate a large number of cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then to a cell with the capacity or potential, under particular circumstances, to differentiate to a more specialized or differentiated phenotype, and which retains the capacity, under certain circumstances, to proliferate without substantially differentiating. In one aspect, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell can derive from a multipotent cell that itself is derived from a multipotent cell, and so on. While each of these multipotent cells can be considered stem cells, the range of cell types that each can give rise to can vary considerably. Some differentiated cells also have the capacity to give rise to cells of greater developmental potential. Such capacity can be natural or can be induced artificially upon treatment with various factors. In many biological instances, stem cells can also be “multipotent” because they can produce progeny of more than one distinct cell type, but this is not required.

Human cells described herein can be induced pluripotent stem cells (IPSCs). An advantage of using iPSCs in the methods of the disclosure is that the cells can be derived from the same subject to which the progenitor cells are to be administered. That is, a somatic cell can be obtained from a subject, reprogrammed to an induced pluripotent stem cell, and then differentiated into a progenitor cell to be administered to the subject (e.g., an autologous cell). Because progenitors are essentially derived from an autologous source, the risk of engraftment rejection or allergic response can be reduced compared to the use of cells from another subject or group of subjects. In addition, the use of iPSCs negates the need for cells obtained from an embryonic source. Thus, in one aspect, the stem cells used in the disclosed methods are not embryonic stem cells.

Methods are known in the art that can be used to generate pluripotent stem cells from somatic cells. Pluripotent stem cells generated by such methods can be used in the method of the disclosure.

Reprogramming methodologies for generating pluripotent cells using defined combinations of transcription factors have been described. Mouse somatic cells can be converted to ES cell-like cells with expanded developmental potential by the direct transduction of Oct4, Sox2, Klf4, and c-Myc; see, e.g., Takahashi and Yamanaka, 2006, Cell 126 (4): 663-76. iPSCs resemble ES cells, as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (see, e.g., Maherali and Hochedlinger, 2008, Cell Stem Cell. 3 (6): 595-605), and tetraploid complementation.

Human iPSCs can be obtained using similar transduction methods, and the transcription factor trio, OCT4, SOX2, and NANOG, has been established as the core set of transcription factors that govern pluripotency; see, e.g., 2014, Budniatzky and Gepstein, Stem Cells Transl Med. 3 (4): 448-57; Barrett et al, 2014, Stem Cells Trans Med 3:1-6 sctm.2014-0121; Focosi et al, 2014, Blood Cancer Journal 4: e211. The production of iPSCs can be achieved by the introduction of nucleic acid sequences encoding stem cell-associated genes into an adult, somatic cell, historically using viral vectors.

iPSCs can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., 2010, Cell Stem Cell, 7 (5): 618-30. Reprogramming can be achieved by introducing a combination of nucleic acids encoding stem cell-associated genes, including, for example, Oct-4 (also known as Oct-3/4 or Pouf51), SoxI, Sox2, Sox3, Sox 15, Sox 18, NANOG, KIfI, KIf2, KIf4, KIf5, NR5A2, c-Myc, 1-Myc, n-Myc, Rem2, Tert, and LIN28. Reprogramming using the methods and compositions described herein can further comprise introducing one or more of Oct-3/4, a member of the Sox family, a member of the Klf family, and a member of the Myc family to a somatic cell. The methods and compositions described herein can further comprise introducing one or more of each of Oct-4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one aspect the reprogramming is not affected by a method that alters the genome. Thus, in such examples, reprogramming can be achieved, e.g., without the use of viral or plasmid vectors.

Efficiency of reprogramming (the number of reprogrammed cells) derived from a population of starting cells can be enhanced by the addition of various agents, e.g., small molecules, as shown by Shi et al., 2008, Cell-Stem Cell 2:525-528; Huangfu et al., 2008, Nature Biotechnology 26 (7): 795-797; and Marson et al., 2008, Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HD AC) inhibitors, valproic acid, 5′-azacytidine, dexamethasone, suberoylanilide, hydroxamic acid (SAHA), vitamin C, and trichostatin (TSA), among others. Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-IH,3H-benzo[de]isoquinolin-2-yl)-N-hydroxybutanamide), Phenylbutyrate (e.g., sodium phenylbutyrate) and Valproic Acid ((VP A) and other short chain fatty acids), Scriptaid, Suramin Sodium, Trichostatin A (TSA), APHA Compound 8, Apicidin, Sodium Butyrate, pi valoyloxy methyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-C1-UCHA (e.g., 6-(3-chlorophenylureido) caproic hydroxamic acid), AOE (2-amino-8-oxo-9, 10-epoxy decanoic acid), CHAP31 and CHAP 50. Other reprogramming enhancing agents include, for example, dominant negative forms of the HDACs (e.g, catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Titan Pharmaceuticals, MethylGene, and Sigma Aldrich.

To confirm the induction of pluripotent stem cells, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, FbxI5, EcatI, EsgI, Eras, Gdfi, Fgf4, Cripto, DaxI, Zpf296, Slc2a3, RexI, UtfI, and NatI. In one case, for example, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. Detection can involve not only RT-PCR, but also detection of protein markers. Intracellular markers can be best identified via RT-PCR, or protein detection methods such as immunocytochemistry, while cell surface markers are readily identified, e.g., by immunocytochemistry.

Pluripotency of isolated cells can be confirmed by tests evaluating the ability of the iPSCs to differentiate into cells of each of the three germ layers. As one example, teratoma formation in nude mice can be used to evaluate the pluripotent character of the isolated clones. The cells can be introduced into nude mice and histology and/or immunohistochemistry can be performed on a tumor arising from the cells. The growth of a tumor comprising cells from all three germ layers, for example, further indicates that the cells are pluripotent stem cells.

Patient-specific iPS cells or cell line can be created. There are many established methods in the art for creating patient specific iPS cells, e.g., as described in Takahashi and Yamanaka 2006; Takahashi, Tanabe et al. 2007. For example, the creating step can comprise: a) isolating a somatic cell, such as a skin cell or fibroblast, from the patient; and b) introducing a set of pluripotency-associated genes into the somatic cell in order to induce the cell to become a pluripotent stem cell. The set of pluripotency-associated genes can be one or more of the genes selected from the group consisting of OCT4, SOX1, SOX2, SOX3, SOX15, SOX18, NANOG, KLF1, KLF2, KLF4, KLF5, c-MYC, n-MYC, REM2, TERT and LIN28.

In some aspects, a biopsy or aspirate of a subject's bone marrow can be performed. A biopsy or aspirate is a sample of tissue or fluid taken from the body. There are many different kinds of biopsies or aspirates. Nearly all of them involve using a sharp tool to remove a small amount of tissue. If the biopsy will be on the skin or other sensitive area, numbing medicine can be applied first. A biopsy or aspirate can be performed according to any of the known methods in the art. For example, in a bone marrow aspirate, a large needle is used to enter the pelvis bone to collect bone marrow.

In some aspects, a mesenchymal stem cell can be isolated from a subject. Mesenchymal stem cells can be isolated according to any method known in the art, such as from a subject's bone marrow or peripheral blood. For example, marrow aspirate can be collected into a syringe with heparin. Cells can be washed and centrifuged on a Percoll™ density gradient. Cells, such as blood cells, liver cells, interstitial cells, macrophages, mast cells, and thymocytes, can be separated using density gradient centrifugation media, Percoll™. The cells can then be cultured in Dulbecco's modified Eagle's medium (DMEM) (low glucose) containing 10% fetal bovine serum (FBS) (Pittinger et. al., 1999, Science 284:143-147).

### 6.8.1. Exemplary Genomic Targets

The Type V Cas proteins and gRNAs of the disclosure can be used to alter various genomic targets. In some aspects, the methods of altering a cell are methods for altering a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence. In some aspects, the methods of altering a cell are methods of altering a TRAC, B2M, PD1, or LAG3 genomic sequence. Reference sequences of RHO, TRAC, B2M, PD1, and LAG3 are available in public databases, for example those maintained by NCBI. For example, RHO has the NCBI gene ID: 6010; TRAC has the NCBI gene ID: 28755; B2M has the NCBI gene ID: 567; PD1 has the NCBI gene ID: 5133; and LAG3 has the NCBI gene ID: 3902.

In some embodiments, the methods of altering a cell are methods for altering a hemoglobin subunit beta (HBB) gene. HBB mutations are associated with B-thalassemia and SCD. Dever et al., 2016 Nature 539 (7629): 384-389.

In some embodiments, the methods of altering a cell are methods for altering a CCR5 gene. CCR5 has demonstrated involvement in several different disease states including, but not limited to, human immunodeficiency virus (HIV) and acquired immune deficiency syndrome (AIDS). WO 2018/119359 describes CCR5 editing by CRISPR-Cas to make loss of function CCR5 in order to provide protection against HIV infection, decrease one or more symptoms of HIV infection, halt or delay progression of HIV to AIDS, and/or decrease one or more symptoms of AIDS.

In some embodiments, the methods of altering a cell are methods for altering a PD1, B2M gene, TRAC gene, or a combination thereof. CAR-T cells having PD1, B2M and TRAC genes disrupted by CRISPR-Type V Cas have demonstrated enhanced activity in preclinical glioma models. Choi et al., 2019, Journal for Immuno Therapy of Cancer 7:309.

In some embodiments, the methods of altering a cell are methods for altering an USH2A gene. Mutations in the USH2A gene can cause Usher syndrome type 2A, which is characterized by progressive hearing and vision loss.

In some embodiments, the methods of altering a cell are methods for altering a RHO gene. Mutations in the RHO gene can cause retinitis pigmentosa (RP).

Targeting of (one or more of) human TRAC, human B2M, human PD1, and human LAG3 genes can be used, for example, in the engineering of chimeric antigen receptor (CAR) T cells. For example, CRISPR/Cas technology has been used to deliver CAR-encoding DNA sequences to loci such as TRAC and PD1 (see, e.g., Eyquem et al., 2017, Nature 543 (7643): 113-117; Hu et al., 2023, eClinicalMedicine 60:102010), while TRAC, B2M, PD1, and LAG3 knockout CAR T-cells have been reported (see, e.g., Dimitri et al., 2022, Molecular Cancer 21:78; Liu et al., 2016, Cell Research 27:154-157; Ren et al., 2017, Clin Cancer Res. 23 (9): 2255-2266; Zhang et al., 2017, Front Med. 11 (4): 554-562). Thus, the Type V Cas proteins and TRAC, B2M, PD1, and LAG3 guides of the disclosure can be used for targeted knock-in of an exogenous DNA sequence to a desired genomic site in a human cell and/or knock-out of TRAC, B2M, PD1, or LAG3 in a human cell, for example a human T cell. In some embodiments, T cells are edited ex vivo to produce CAR-T cells and subsequently administered to a subject in need of CAR-T cell therapy.

In some embodiments, the methods of altering a cell are methods for altering a DNMT1 gene. Mutations in the DNMT1 gene can cause DNMT1-related disorder, which is a degenerative disorder of the central and peripheral nervous systems. DNMT1-related disorder is characterized by sensory impairment, loss of sweating, dementia, and hearing loss.

Additional exemplary targets include AVS1, BCL11A, PCSK9, and VEGFA. In some embodiments, the methods of altering a cell are methods for altering an AVS1 gene. AVS1 can be used as a safe harbor locus to insert an transgene of interest (see, e.g., Gu et al., 2022, Methods Mol Biol. 2495:99-114). In some embodiments, the methods of altering a cell are methods for altering a BCL11A gene. Editing BCL11A has been identified in the art a target for treatment of sickle cell disease and β-Thalassemia (see, e.g., Frangoul et al., 2021, N Eng J Med 384:252-260). In some embodiments, the methods of altering a cell are methods for altering a PCSK9 gene. PCSK9 has been identified in the art as a target for treatment of hypercholesterolemia (see, e.g., Hoekstra & Van Eck, 2024, Current Atherosclerosis Reports, 26:139-146). In some embodiments, the methods of altering a cell are methods for altering a VEGFA gene. VEGFA has been identified in the art as a target for treatment of eye diseases such as age-related macular degeneration (see, e.g., Park et al., 2023, Scientific Reports 13:3715).

### 6.9. Methods of Detecting Target Nucleic Acids

The disclosure further provides methods of using the Type V Cas proteins, gRNAs, and systems of the disclosure for detecting target nucleic acids (e.g., nucleic acids from pathogens, for example viruses, bacteria, or parasites). Nucleic acid detection methods using Cas12a are described in the art (see, e.g., Kaminski et al., 2021, Nature Biomedical Engineering 5:643-656; Sashital, 2018, Genome Med. 10:32, each of which is incorporated herein by reference in its entirety), and such methods can be extended to the Type V Cas proteins of the disclosure. Nucleic acid detection methods typically take advantage of collateral cleavage activity of Type V Cas proteins. For example, target binding of Type V Cas proteins such as Cas12a activates collateral cleavage activity toward single-stranded DNA, and this activity can be exploited in a detection assay by supplying a single-stranded reporter nucleic acid, for example a reporter nucleic acid comprising a quenched fluorescent reporter. Type V Cas protein binding to the target nucleic acid leads to cleavage of the reporter nucleic acid. Detection of the fluorescent reporter following cleavage of the reporter nucleic acid allows for detection and, optionally, quantification of the target nucleic acid.

### 7. EXAMPLES

### 7.1. Materials and Methods

### 7.1.1. Plasmids and Cell Lines

Plasmids: Type V-A Cas proteins were expressed in mammalian cells from a plasmid vector characterized by a EF1alpha-driven cassette. Each Type V-A Cas protein coding sequence was human codon-optimized and modified by the addition of an SV5 tag and a bipartite nuclear localization signal at the C-terminus. Additional constructs containing different NLS configurations (discussed in Section 7.4.2) were generated using standard cloning techniques. The crRNA were expressed from a U6-driven cassette located on an independent plasmid construct. The human codon-optimized coding sequence of the Type V-A Cas proteins, as well as their crRNA scaffolds, were obtained by synthesis from Twist Bioscience. Spacer sequences (20-24 nt long) were cloned into the crRNA plasmid as annealed DNA oligonucleotides (Eurofins Genomics) using a double BsaI site present in the plasmid. The list of spacer sequences and relative cloning oligonucleotides used in the present example is reported in Table 8. In all cases in which the crRNA scaffold did not contain a matching native 5′-G, this nucleotide was appended upstream the scaffold sequence in order to allow efficient transcription from a U6 promoter. Unless otherwise stated in all studies, full-length crRNAs were used.

TABLE 8
Spacer sequences and oligonucleotides relative to crRNAs for Type V-A Cas proteins

| | | | SEQ | | | SEQ | | SEQ |
|---|---|---|---|---|---|---|---|---|
| | | Spacer | ID | PAM | Oligo 1 | ID | Oligo 2 | ID |
| Guide ID | Target | (5′→3′) | NO: | (5′→3′) | (5′→3′) | NO: | (5′→3′) | NO: |
| EGFP-g1 | EGFP | CGUCGCCGUCCA | 260 | TTTA | agatCGTCGCCGTC | 262 | AaaaCCTGGTCGAG | 308 |
| | | GCUCGACCAGG | | | CAGCTCGACCAGG | | CTGGACGGCGACG | |
| EGFP-g2 | EGFP | CUCAGGGGGGA | 261 | TTTG | agatCTCAGGGCGG | 263 | AaaaCTGAGCACCC | 309 |
| | | CUGGGUGCUCA | | | ACTGGGTGCTCAG | | AGTCCGCCCTGAG | |
| | | G | | | | | | |
| B2M-g1 | B2M | UGGCCUGGAGG | 164 | TTTC | agatTGGCCTGGAG | 264 | aaaaCACGCTGGATA | 310 |
| | | CUAUCCAGCGUG | | | GCTATCCAGCGTG | | GCCTCCAGGCCA | |
| B2M-g2 | B2M | CUCACGUCAUCC | 165 | TTTC | agatCTCACGTCATC | 265 | aaaaCATTCTCTGCT | 311 |
| | | AGCAGAGAAUG | | | CAGCAGAGAATG | | GGATGACGTGAG | |
| B2M-g3 | B2M | ACUUUCCAUUCU | 166 | TTTG | agatACTTTCCATTC | 266 | aaaaTCATCCAGCAG | 312 |
| | | CUGCUGGAUGA | | | TCTGCTGGATGA | | AGAATGGAAAGT | |
| B2M-g4 | B2M | CUGAAUUGCUAU | 167 | TTTC | agatCTGAATTGCTA | 267 | aaaaAACCCAGACAC | 313 |
| | | GUGUCUGGGUU | | | TGTGTCTGGGTT | | ATAGCAATTCAG | |
| B2M-g5 | B2M | AAUUCUCUCUCC | 168 | TTTC | agatAATTCTCTCTC | 268 | aaaaTACTGAAGAAT | 314 |
| | | AUUCUUCAGUA | | | CATTCTTCAGTA | | GGAGAGAGAATT | |
| TRAC-g1 | TRAC | AGAAUCAAAAUC | 169 | TTTA | agatAGAATCAAAAT | 269 | aaaaGCCTATTCACC | 315 |
| | | GGUGAAUAGGC | | | CGGTGAATAGGC | | GATTTTGATTCT | |
| TRAC-g2 | TRAC | UGACACAUUUGU | 170 | TTTG | agatTGACACATTTG | 270 | aaaaTTGATTCTCAA | 316 |
| | | UUGAGAAUCAA | | | TTTGAGAATCAA | | ACAAATGTGTCA | |
| TRAC-g3 | TRAC | GAGUCUCUCAGC | 171 | TTTA | agatGAGTCTCTCA | 271 | aaaaGCCGTGTACCA | 317 |
| | | UGGUACACGGC | | | GCTGGTACACGGC | | GCTGAGAGACTC | |
| TRAC-g4 | TRAC | UCUGUGAUAUAC | 172 | TTTG | agatTCTGTGATATA | 272 | aaaaGATTCTGATGT | 318 |
| | | ACAUCAGAAUC | | | CACATCAGAATC | | GTATATCACAGA | |
| TRAC-g5 | TRAC | AUUCUCAAACAA | 173 | TTTG | agatATTCTCAAACA | 273 | aaaaTTGTGACACAT | 319 |
| | | AUGUGUCACAA | | | AATGTGTCACAA | | TTGTTTGAGAAT | |
| PD1-g1 | PD1 | CCUUCCGCUCAC | 174 | TTTC | agatCCTTCCGCTC | 274 | aaaaCTCAGGCGGA | 320 |
| | | CUCCGCCUGAG | | | ACCTCCGCCTGAG | | GGTGAGCGGAAGG | |
| PD1-g2 | PD1 | GCACGAAGCUCU | 175 | TTTA | agatGCACGAAGCT | 275 | aaaaCAACACATCGG | 321 |
| | | CCGAUGUGUUG | | | CTCCGATGTGTTG | | AGAGCTTCGTGC | |
| PD1-g3 | PD1 | AUCUGCGCCUUG | 176 | TTTG | agatATCTGCGCCTT | 276 | aaaaTCCCTGGCCCC | 322 |
| | | GGGGCCAGGGA | | | GGGGGCCAGGGA | | CAAGGCGCAGAT | |
| PD1-g4 | PD1 | GAACUGGCCGG | 177 | TTTG | agatGAACTGGCCG | 277 | aaaaCACCCAGGCC | 323 |
| | | CUGGCCUGGGU | | | GCTGGCCTGGGTG | | AGCCGGCCAGTTC | |
| | | G | | | | | | |
| AAVS1- | AAVS1 | CAGGGGUCCGA | 195 | CTTC | agatCAGGGGTCCG | 278 | aaaaCTAGCTGAGCT | 324 |
| g6 | | GAGCUCAGCUAG | | | AGAGCTCAGCTAG | | CTCGGACCCCTG | |
| AAVS1- | AAVS1 | AUCUGUCCCCUC | 196 | TTTT | agatATCTGTCCCCT | 279 | aaaaACTGTGGGGT | 325 |
| g7 | | CACCCCACAGU | | | CCACCCCACAGT | | GGAGGGGACAGAT | |
| AAVS1- | AAVS1 | GGCAGCUCCCCU | 194 | TTTG | agatGGCAGCTCCC | 280 | aaaaGTAAGGGGGG | 326 |
| g2 | | ACCCCCCUUAC | | | CTACCCCCCTTAC | | TAGGGGAGCTGCC | |
| B2M-g8 | B2M | GUGUCAAGCUAU | 181 | CTTG | agatGTGTCAAGCT | 281 | aaaaTGGTGCCTGAT | 327 |
| | | AUCAGGCACCA | | | ATATCAGGCACCA | | ATAGCTTGACAC | |
| B2M-g9 | B2M | AUGUGUCUUUUC | 182 | ATTA | agatATGTGTCTTTT | 282 | aaaaAGGAATATCGG | 328 |
| | | CCGAUAUUCCU | | | CCCGATATTCCT | | GAAAAGACACAT | |
| TRAC-g6 | TRAC | UCACUGGAUUUA | 184 | CTTG | agatTCACTGGATTT | 283 | aaaaCTGAGAGACTC | 329 |
| | | GAGUCUCUCAG | | | AGAGTCTCTCAG | | TAAATCCAGTGA | |
| TRAC-g9 | TRAC | GAUUCUCAAACA | 185 | TTTT | agatGATTCTCAAAC | 284 | aaaaTCACTGGATTT | 330 |
| | | AAUGUGUCACA | | | AAATGTGTCACA | | AGAGTCTCTCAG | |
| TRAC- | TRAC | AAGAGGGAAAUG | 186 | GTTA | agatAAGAGGGAAA | 285 | aaaaGGACATGATCT | 331 |
| g11 | | AGAUCAUGUCC | | | TGAGATCATGTCC | | CATTTCCCTCTT | |
| TRAC- | TRAC | ACCGAUUUUGAU | 187 | ATTC | agatACCGATTTTGA | 286 | aaaaTTTGTTTGAGA | 332 |
| g13 | | UCUCAAACAAA | | | TTCTCAAACAAA | | ATCAAAATCGGT | |
| TRAC- | TRAC | GUCUGUGAUAUA | 188 | TTTT | agatGTCTGTGATAT | 287 | aaaaATTCTGATGTG | 333 |
| g15 | | CACAUCAGAAU | | | ACACATCAGAAT | | TATATCACAGAC | |
| BCL11A- | BCL11A | AGCCAUCUCACU | 199 | TTTC | agatAGCCATCTCA | 288 | aaaaGAGTTATCTGT | 334 |
| g1 | | ACAGAUAACUC | | | CTACAGATAACTC | | AGTGAGATGGCT | |
| AAVS1- | AAVS1 | AUUUGGGCAGCU | 193 | TTTC | agatATTTGGGCAG | 289 | aaaaGGGGGTAGGG | 335 |
| g1 | | CCCCUACCCCC | | | CTCCCCTACCCCC | | GAGCTGCCCAAAT | |
| EMX1-g2 | EMX1 | UACUUUGUCCUC | 197 | TTTG | agatTACTTTGTCCT | 290 | aaaaTTCCAGAACCG | 336 |
| | | CGGUUCUGGAA | | | CCGGTTCTGGAA | | GAGGACAAAGTA | |
| EMX1-g3 | EMX1 | UCCUCCGGUUCU | 198 | TTTG | agatTCCTCCGGTT | 291 | aaaaGGTGTGGTTCC | 337 |
| | | GGAACCACACC | | | CTGGAACCACACC | | AGAACCGGAGGA | |
| BCL11A- | BCL11A | AAGCUAGUCUAG | 200 | TTTG | agatAAGCTAGTCTA | 292 | aaaaGTTAGCTTGCA | 338 |
| g2 | | UGCAAGCUAAC | | | GTGCAAGCTAAC | | CTAGACTAGCTT | |
| BCL11A- | BCL11A | GCCUCUGAUUAG | 201 | TTTG | agatGCCTCTGATTA | 293 | aaaaACGCCCCCAC | 339 |
| g3 | | GGUGGGGGCGU | | | GGGTGGGGGCGT | | CCTAATCAGAGGC | |
| PCSK9- | PCSK9 | UCUGCCACCCAC | 203 | TTTC | agatTCTGCCACCC | 294 | aaaaAAGGTGAGGA | 340 |
| g1 | | CUCCUCACCUU | | | ACCTCCTCACCTT | | GGTGGGTGGCAGA | |
| PCSK9- | PSCK9 | CAGGUCAUCACA | 204 | TTTC | agatCAGGTCATCA | 295 | aaaaGTGGCCCCAA | 341 |
| g2 | | GUUGGGGCCAC | | | CAGTTGGGGCCAC | | CTGTGATGACCTG | |
| BCL11A- | BCL11A | UCACAGGCUCCA | 202 | TTTA | agatTCACAGGCTC | 296 | aaaaAACCCTTCCTG | 342 |
| g4 | | GGAAGGGUU | | | CAGGAAGGGTT | | GAGCCTGTGA | |
| VEGFA- | VEGFA | GAGAGUGAGGAC | 205 | CTTC | agatGAGAGTGAGG | 297 | aaaaGACACACACGT | 343 |
| g1 | | GUGUGUGUC | | | ACGTGTGTGTC | | CCTCACTCTC | |
| B2M- | B2M | UGGCCUGGAGG | 183 | TTTC | agatTGGCCTGGAG | 298 | aaaaCGCTGGATAGC | 344 |
| g1_21nt | | CUAUCCAGCG | | | GCTATCCAGCG | | CTCCAGGCCA | |
| TRAC | TRAC | GAGUCUCUCAGC | 189 | TTTA | AGATGAGTCTCTC | 299 | AAAAGTGTACCAGC | 345 |
| g3_20 nt | | UGGUACAC | | | AGCTGGTACAC | | TGAGAGACTC | |
| TRAC | TRAC | GAGUCUCUCAGC | 190 | TTTA | AGATGAGTCTCTC | 300 | AAAACGTGTACCAG | 346 |
| g3_21 nt | | UGGUACACG | | | AGCTGGTACACG | | CTGAGAGACTC | |
| TRAC | TRAC | GAGUCUCUCAGC | 191 | TTTA | AGATGAGTCTCTC | 301 | AAAACCGTGTACCA | 347 |
| g3_22 nt | | UGGUACACGG | | | AGCTGGTACACGG | | GCTGAGAGACTC | |
| TRAC | TRAC | GAGUCUCUCAGC | 192 | TTTA | AGATGAGTCTCTC | 302 | AAAATGCCGTGTAC | 348 |
| g3_24 nt | | UGGUACACGGCA | | | AGCTGGTACACGG | | CAGCTGAGAGACTC | |
| | | | | | CA | | | |
| Match6_ | Match6 | GGGUGAUCAGAC | 206 | TTTG | AGATGGGTGATCA | 303 | AAAAGCTGTTGGGT | 349 |
| 20 nt | | CCAACAGC | | | GACCCAACAGC | | CTGATCACCC | |
| Match6_ | Match6 | GGGUGAUCAGAC | 207 | TTTG | AGATGGGTGATCA | 304 | AAAATGCTGTTGGG | 350 |
| 21 nt | | CCAACAGCA | | | GACCCAACAGCA | | TCTGATCACCC | |
| Match6 | Match6 | GGGUGAUCAGAC | 208 | TTTG | AGATGGGTGATCA | 305 | AAAACTGCTGTTGG | 351 |
| 22 nt | | CCAACAGCAG | | | GACCCAACAGCAG | | GTCTGATCACCC | |
| Match6_ | Match6 | GGGUGAUCAGAC | 209 | TTTG | AGATGGGTGATCA | 306 | AAAATGCTGTTGGG | 350 |
| 23 nt | | CCAACAGCAGG | | | GACCCAACAGCAG | | TCTGATCACCC | |
| | | | | | G | | | |
| Match6_ | Match6 | GGGUGAUCAGAC | 210 | TTTG | AGATGGGTGATCA | 307 | AAAAACCTGCTGTT | 352 |
| 24 nt | | CCAACAGCAGGU | | | GACCCAACAGCAG | | GGGTCTGATCACCC | |
| | | | | | GT | | | |

Cell lines: U2OS-EGFP cells, harboring a single integrated copy of an EGFP reporter gene, and wild-type U2OS and HEK293T cells were cultured in DMEM (Life Technologies) supplemented with 10% FBS (Life Technologies), 2 mM L-Glutamine (Life Technologies) and penicillin/streptomycin (Thermo Fisher). All cells were incubated at 37° C. and 5% CO₂ in a humidified atmosphere. All cells tested mycoplasma negative (PlasmoTest, Invivogen).

7.1.2. Identification of Novel Type V-A Cas Molecules from Metagenomic Samples

Type V CRISPR-Cas loci were predicted using CRISPRCasTyper (Russel, J., Pinilla-Redondo, R., Mayo-Muñoz, D., Shah, S. A. & Sørensen, S. J. CRISPRCasTyper: Automated Identification, Annotation, and Classification of CRISPR-Cas Loci. CRISPR J 3, 462-469 (2020)) version 1.8.0, starting from a collection of >1M metagenome-assembled genomes (MAGs) and reference genomes (Blanco-Míguez, A. et al. Extending and improving metagenomic taxonomic profiling with uncharacterized species using MetaPhlAn 4. Nat. Biotechnol. 41, 1633-1644 (2023)). A total of 14,568 Type V Cas proteins were recovered. Type V Cas proteins were clustered at 60% sequence identity and 60% sequence coverage using MMseq2 (Steinegger, M. & Söding, J. MMseqs2 enables sensitive protein sequence searching for the analysis of massive data sets. Nat. Biotechnol. 35, 1026-1028 (2017)) version 13.45111 (-c 0.6--cov-mode 5--min-seq-id 0.6--cluster-reassign) and aligned using mafft (Katoh, K. & Standley, D. M. MAFFT multiple sequence alignment software version 7: improvements in performance and usability. Mol. Biol. Evol. 30, 772-780 (2013)) version 7.490 (--maxiterate 100). The resulting alignment was trimmed using TrimAl (Capella-Gutiérrez, S., Silla-Martínez, J. M. & Gabaldón, T. trimAI: a tool for automated alignment trimming in large-scale phylogenetic analyses. Bioinformatics 25, 1972-1973 (2009)) version 1.4.rev15 (-gappyout) and used to generate a phylogenetic tree using IQ-TREE 2 (Minh, B. Q. et al. IQ-TREE 2: New Models and Efficient Methods for Phylogenetic Inference in the Genomic Era. Mol. Biol. Evol. 37, 1530-1534 (2020)) version 2.0.3 (-B 1000) and automatic model selection (Kalyaanamoorthy, S., Minh, B. Q., Wong, T. K. F., von Haeseler, A. & Jermiin, L. S. ModelFinder: fast model selection for accurate phylogenetic estimates. Nat. Methods 14, 587-589 (2017)), which was visualized using GraPhIAn (Asnicar, F., Weingart, G., Tickle, T. L., Huttenhower, C. & Segata, N. Compact graphical representation of phylogenetic data and metadata with GraPhIAn. PeerJ 3, e1029 (2015)) version 1.1.3. PAM predictions were performed using PAMpredict (Ciciani, M. et al. Automated identification of sequence-tailored Cas9 proteins using massive metagenomic data. Nat. Commun. 13, 6474 (2022)), clustering Type V-A Cas proteins at 90% sequence identity. For selected Type V-A Cas proteins, crRNAs resulting from MinCED predictions (Bland, C. et al. CRISPR recognition tool (CRT): a tool for automatic detection of clustered regularly interspaced palindromic repeats. BMC Bioinformatics 8, 209 (2007)) were manually checked for conservation of the 3′ end sequence. The structure of the 3′ end was checked by aligning the crRNAs using Clustal Omega (Sievers, F. et al. Fast, scalable generation of high-quality protein multiple sequence alignments using Clustal Omega. Mol. Syst. Biol. 7, 539 (2011)) version 1.2.4, generating a consensus secondary structure with RNAalifold version 2.4. 17 (-p-r-d2--noLP) (Lorenz, R. et al. ViennaRNA Package 2.0. Algorithms Mol. Biol. 6, 26 (2011)) and analyzing the resulting structure with R2R (Weinberg, Z. & Breaker, R. R. R2R--software to speed the depiction of aesthetic consensus RNA secondary structures. BMC Bioinformatics 12, 3 (2011)) version 1.0.6.

### 7.1.3. PAM Assay

An in vitro PAM evaluation of the novel Type V-A Cas proteins was performed according to a modified version of the protocol from Karvelis, Young and Siksnys (Karvelis et al., 2019, Methods in Enzymology 616:219-240). The gRNAs to perform the assay were obtained by in vitro transcription using the HighYield™ T7 RNA Synthesis Kit (Jena Bioscience) starting from a PCR template generated by amplification from each gRNA expression construct. The primers used to generate the IVT templates are reported in Table 9. In vitro transcribed gRNAs were subsequently purified using the MEGAClear™ Transcription Clean-up kit (Thermo Fisher Scientific). HEK293T cells were transfected 48 hours before the study with nuclease-expressing plasmids, and protein lysates were collected and used for RNP complex formation. The complex was assembled by combining 20 μL of the supernatant containing the soluble Type V-A Cas proteins with 1 μL of RiboLock™ RNase Inhibitor (Thermo Fisher Scientific) and 2 ug of guide RNAs (previously transcribed in vitro). The RNP complex was used to digest 1 μg of a PAM plasmid DNA library (containing a defined target sequence flanked at the 5′-end by a randomized 8 nucleotide PAM sequence) for 1 hour at 37° C.

A double stranded DNA adapter (Table 10) was ligated to the DNA ends generated by the targeted Type V-A Cas protein cleavage and the final ligation product was purified using CleanNGS™ SPRI beads.

One round of a two-step PCR (Phusion™ HF DNA polymerase, Thermo Fisher Scientific) was performed to enrich the sequences that were cut using a set of forward primers annealing on the adapter and a reverse primer designed on the plasmid backbone downstream of the PAM (Table 11). A second round of PCR was performed to attach the Illumina indexes and adapters. PCR products were purified using the GeneJet™ PCR Purification Kit (Thermo Fisher Scientific).

The library was analysed with a 71-bp single read sequencing, using a flow cell v2 micro, on an Illumina MiSeq™ sequencer.

PAM sequences were extracted from Illumina MiSeq reads and used to generate PAM sequence logos, using Logomaker version 0.8. PAM heatmaps were used to display PAM enrichment, computed dividing the frequency of PAM sequences in the cleaved library by the frequency of the same sequences in a control uncleaved library.

TABLE 9
Sequences of the primers used for PCR amplification of gRNAs used as templates for
in vitro transcription

| | | SEQ ID |
|---|---|---|
| Primer name | Sequence (5′→3′) | NO: |
| ZZKD_PAMassay_F | CCTCTAATACGACTCACTATAGCCTTTGGAAGTACTAAGAATTTCTAC | 353 |
| | TGTTGTAGATAGGTGAAGTTCGAGGGCGACGAA | |
| ZZKD_PAMassay_R | TTCGTCGCCCTCGAACTTCACCTATCTACAACAGTAGAAATTCTTAGT | 354 |
| | ACTTCCAAAGGCTATAGTGAGTCGTATTAGAGG | |
| ZZQE_PAMassay_F | cctcTAATACGACTCACTATAGGCTACTAAGCCTTTATAATTTCTACTAT | 355 |
| | TGTAGATAGGTGAAGTTCGAGGGCGACgaa | |
| ZZQE_PAMassay_R | ttcGTCGCCCTCGAACTTCACCTATCTACAATAGTAGAAATTATAAAGG | 356 |
| | CTTAGTAGCCTATAGTGAGTCGTATTAgagg | |
| ZRGM_PAMassay_F | cctcTAATACGACTCACTATAGTCTGAAAGACTATATAATTTCTACTTCG | 357 |
| | TGTAGATAGGTGAAGTTCGAGGGCGACgaa | |
| ZRGM_PAMassay_R | ttcGTCGCCCTCGAACTTCACCTATCTACACGAAGTAGAAATTATATAG | 358 |
| | TCTTTCAGACTATAGTGAGTCGTATTAgagg | |

TABLE 10
Sequences of the two oligonucleotides used to prepare the dsDNA
adapter for the in vitro PAM assay

| Name | Sequence (5′→3′) | SEQ ID NO: |
|---|---|---|
| Oligo UP | CGGCATTCCTGCTGAACCGCTCTTCCGATCT | 359 |
| Oligo BOTTOM | GATCGGAAGAGCGGTTCAGCAGGAATGCCG | 360 |

TABLE 11
Sequences of the primers used for NGS
library preparation in the in vitro PAM assay

| Primer | | SEQ ID |
|---|---|---|
| name | Sequence (5′→3′) | NO: |
| F4a | TCGTCGGCAGCGTCAGATGTGTATAAGAGACAGC | 361 |
| | TGCTGAACCGCTCTTCCGATC | |
| F4b | TCGTCGGCAGCGTCAGATGTGTATAAGAGACAGT | 362 |
| | AAGACTGCTGAACCGCTCTTCCGATC | |
| F4c | TCGTCGGCAGCGTCAGATGTGTATAAGAGACAGG | 363 |
| | CTAGACCTAATGTGATCTGCTGAACCGCTCTTCC | |
| | GATC | |
| R3 | GTCTCGTGGGCTCGGAGATGTGTATAAGAGACAG | 364 |
| | TCTGCGTTCTGATTTAATCTGTATCAGGC | |

### 7.1.4. In Vitro Cleavage Assays

In vitro cleavage assays were performed using an RNP complex targeting a PCR product. Briefly, the RNP was assembled combining 105.7 pmol of synthetic RNA with 35 pmol of protein (ratio 3:1) and the complex was incubated 15 min at room temperature (approximately 20-22° C.). Two ug of PCR template was diluted in 90 μl of R buffer (10 Mm Tris-HCl PH 7.5; 10 mM NaCl; 1 mM DTT) and mixed with 9 μl of RNP complex. The reaction was incubated at 37° C. for 1 hour and then run on 1% agarose gel. Digested bands were gel-extracted and purified using a commercial kit (Macherey-Nagel), and sent for Sanger sequencing using the primers TRAC_ex1 forward and TRAC_ex1 reverse reported in Table 12.

### 7.1.5. Cell Line Transfections

For studies in HEK293T cells, 100,000 cells were plated in a 24 well plate. 24 hours later, cells were transfected with 500 ng of nuclease-expressing plasmid and 250 ng of sgRNA-expressing plasmid using Mirus TransIT™-LT1 according to the manufacturer's instructions. After 15-30 minutes of incubation at room temperature, the mixture was added drop-wise on HEK293T cultures.

To perform editing studies, 200,000 U2OS-EGFP cells were nucleofected with 500 ng of nuclease-expressing plasmid and 250 ng of sgRNA-expressing plasmid containing a guide designed to target EGFP using the 4D-Nucleofector™ SE Kit (Lonza), DN-100 program, according to the manufacturer's protocol. After electroporation, cells were plated in a 24-well plate. EGFP knock-out was analyzed 4 days after nucleofection using a BD FACSymphony™ A1 (BD) flow cytometer.

### 7.1.6. RNP Electroporation

200,000 U2OS cells were electroporated with RNP complexes (450 pmol of crRNAs+150 pmol of recombinant ZZKD Type V-A Cas protein) pre-formed at room temperature for 20 minutes using the 4D-Nucleofector™ SE Kit (Lonza), DN-100 program, according to the manufacturer's protocol. For RNP electroporation studies in primary human T cells, commercial lots were purchased from CGT preclinical. Briefly, a vial of 10×10⁶ T cells, was thawed and incubated in RPMI+100 U/mL IL-2 (ImmunoTools). Four hours later, the T cells were counted, spun down, and resuspended in 5 mL of activation media (RPMI+IL-2 100 U/mL+100 μL TransAct T cell activator from Miltenyi Biotech), resulting in 10 million cells at a concentration of 2 million cells/mL. Three days post-activation, activated T cells were electroporated using Lonza 4D-Nucleofector™, EO115 program, with a pre-assembled RNP complex generated by mixing 450 pmol of the ZZKD Type V-A Cas protein and 150 pmol of the sgRNA and kept at room temperature for 20 minutes before electroporation. KO efficiency was evaluated 4 days post-electroporation by staining the T cells with an anti-human TCR alpha/beta chain antibody (BioLegend) for 30 minutes at 4° C. and quantifying the percentage of negative cells via flow cytometry. The recombinant ZZKD Type V-A protein was custom-produced by Origene, starting from a 6-His tagged (SEQ ID NO: 365) bacterial expression construct generated by gene synthesis (Twist Bioscience), while synthetic guide RNAs were purchased from IDT.

### 7.1.7. Evaluation of Gene Editing

Three days after transfection cells were collected and DNA was extracted using the QuickExtract™ DNA Extraction Solution (Lucigen) according to the manufacturer's instructions. To amplify the target loci, PCR reactions were performed using the HOT FIREPol™ polymerase (Solis BioDyne) and the oligonucleotides listed in Table 12. The amplified products were purified, sent for Sanger sequencing (EasyRun service, Microsynth) and analyzed with the TIDE web tool (shinyapps.datacurators.nl/tide/) to quantify indels. The forward primers used for generating the amplicons were also exploited for Sanger sequencing reactions.

TABLE 12
Primers used to amplify target loci for Sanger sequencing

| | | SEQ ID | | SEQ ID |
|---|---|---|---|---|
| Target | Forward oligo (5′→3′) | NO: | Reverse oligo (5′→3′) | NO: |
| TRAC_ex1 | CATCACGAGCAGCTGGTTTC | 366 | TGGCAATGGATAAGGCCGAG | 378 |
| B2M_ex1 | CTCTAACCTGGCACTGCGTC | 367 | GGTGCTAGGACATGCGAACTTAG | 379 |
| B2M_ex2 | TGGCCAGAGTGGAAATGGAA | 368 | TGTATTTGTGCAAGTGCTGCT | 380 |
| PD1_ex1 | CACTGCCTCTGTCACTCTCG | 369 | TGGGGCTCCCATCCTTA | 381 |
| PD1_ex2 | CCTCACGTAGAAGGAAGAGGC | 370 | AGAGATGCCGGTCACCATTC | 382 |
| PD1_ex3_F | AATGGTGACCGGCATCTCTG | 371 | AAGGCACAGTGGATCATGCA | 383 |
| AAVS1 | CCTTATATTCCCAGGGCCGG | 372 | GAGAAAGGGAGTAGAGGCGG | 384 |
| VEGFA_2 | ACTTTGATGTCTGCAGGCCA | 373 | GAGCCTCAGCCCTTCCAC | 385 |
| EMX1 | ATTTCGGACTACCCTGAGGAG | 374 | GGAATCTACCACCCCAGGCTCT | 386 |
| Match6 | TGCTAGACTTGCTGCTCCTT | 375 | TGAAGGGATTGTGCTGGTGT | 387 |
| PCSK9 | TGAACTTCAGCTCCTGCACA | 376 | TGCAGTTCCCAGTACGTTCC | 388 |
| BCL11A | GCATCACAACAGGCAGAGAAT | 377 | TATGACGTCAGGGGGAGGCAAG | 389 |
| | GTC | | TC | |

### 7.2. Example 1: Identification and Characterization of Novel Type V-A Cas Molecules

This Example describes studies performed to identify and characterize ZWGD, ZJHK, ZIKV, ZZFT, YYAN, ZZGY, ZKBG, ZZKD, ZXPB, and ZPPX TYPE V-A Cas proteins.

7.2.1. Identification of the crRNAs of Novel Type V-A Cas Proteins

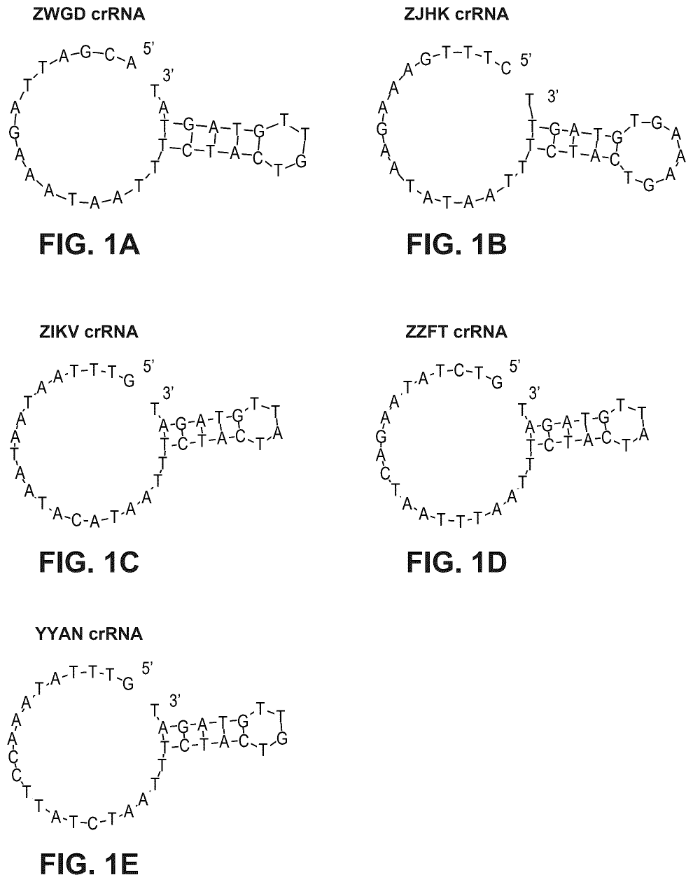

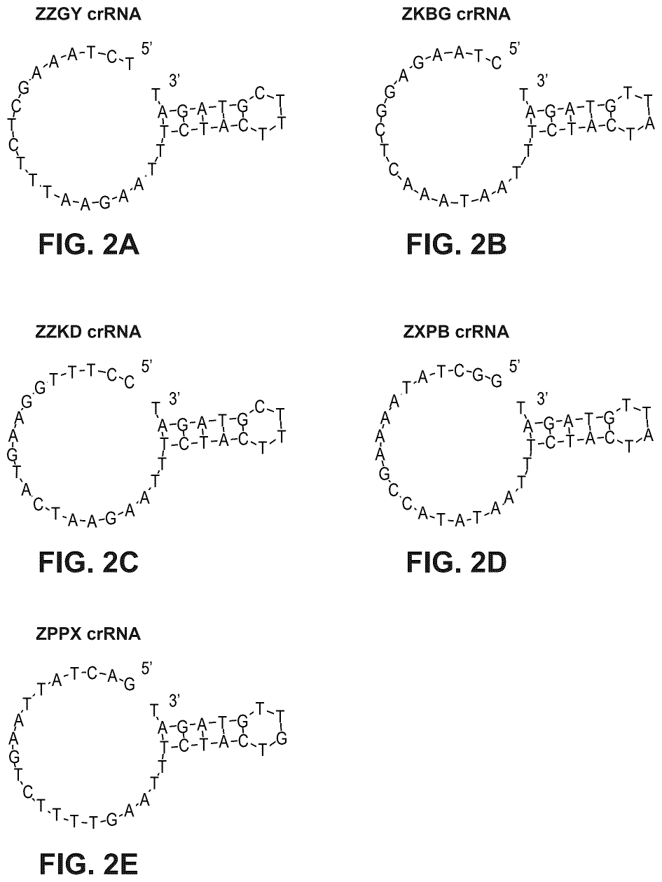

crRNA sequences for the selected Type V-A Cas proteins were identified in silico by extracting the repeat region of the CRISPR arrays associated with each nuclease, as described in the Materials & Methods (Section 7.1). The secondary structures of the identified cRNAs for each of the Type V-A Cas proteins are reported in FIGS. 1A-1E and FIGS. 2A-2E.

### 7.2.2. In Silico Prediction of the PAM Specificity of Novel Type V-A Cas Proteins

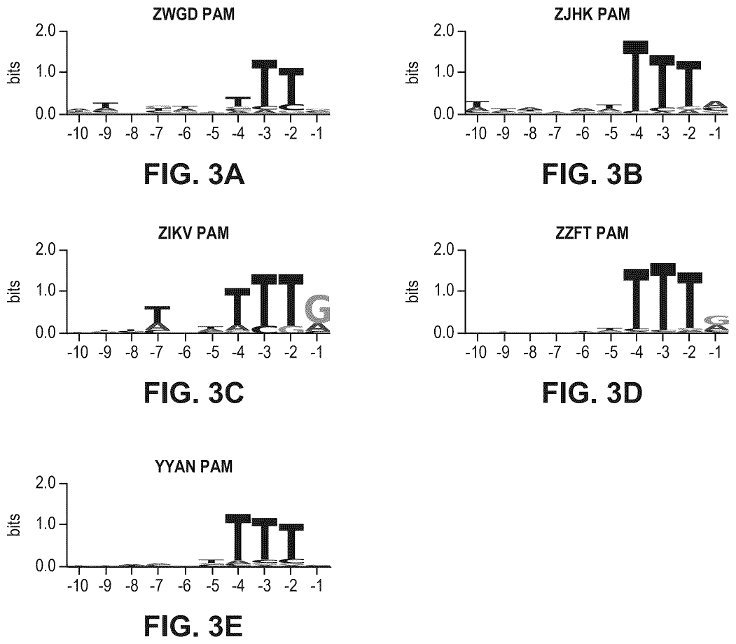

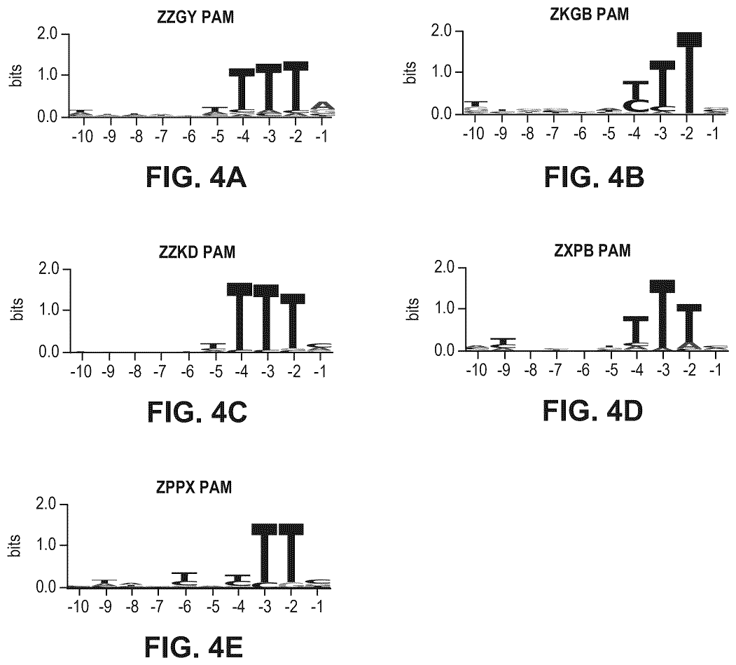

An in silico PAM prediction pipeline (as reported above in the Materials & Methods (Section 7.1)) has been used to predict the PAM recognition specificity of the novel Type V-A Cas proteins. Table 13 reported here below contains the PAM preferences as predicted by the algorithm. The predicted PAM logos for each enzyme are reported in FIGS. 3A-3E and 4A-4E.

TABLE 13
In silico predicted PAM sequences for selected
Type V-A Cas proteins

| Type V-A Cas protein | Predicted PAM (5′→3′) |
|---|---|
| ZWGD Type V-A Cas | TTTN, TTN |
| ZJHK Type V-A Cas | TTTN, TTTV |
| ZIKV Type V-A Cas | TTTR, TNNTTTR, DNNTTTR |
| ZZFT Type V-A Cas | TTTR |
| YYAN Type V-A Cas | TTTN |
| ZZGY Type V-A Cas | TTTN, TTTR |
| ZKBG Type V-A Cas | YTTN, TTTN |
| ZZKD Type V-A Cas | TTTN, TTTV |
| ZXPB Type V-A Cas | TTTN, DTTN, DTDN |
| ZPPX Type V-A Cas | YTTN, TTTN |

### 7.2.3. Evaluation of Type V-A Cas Proteins Editing Activity Using an EGFP Reporter System

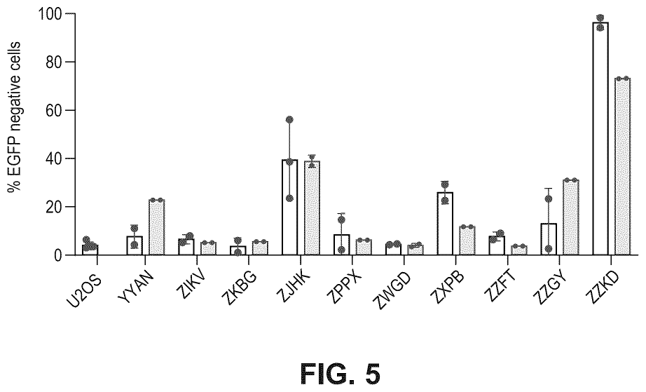

By exploiting the knowledge on their predicted PAM sequences and their identified crRNAs, the ability to cleave selected targets in mammalian cells of the selected Type V-A Cas proteins was investigated. An EGFP reporter system was used as it allowed an easier readout on the editing activity, based on the loss of fluorescence of treated cells quantitatively measured by cytofluorimetry. Two gRNAs targeting the EGFP coding sequence were designed exploiting PAMs which, based on the in silico prediction, were compatible for all the Type V-A Cas proteins and tested in U2OS cells stably expressing a single copy of an EGFP reporter by transient electroporation. Surprisingly, as reported in FIG. 5, some of the evaluated guides in combination with their respective Type V-A Cas protein were able to significantly downregulate EGFP expression in target cells. In particular, ZZKD Type V-A Cas protein showed very high activity with both of the guides (>70 and >95% EGFP KO); additionally, ZJHK, ZZGY, ZXPB and YYAN Type V-A Cas proteins showed appreciable knock-out activity (>20% EGFP KO) with at least one of the gRNAs. The remaining Type V-A Cas proteins did not show editing levels above the background of the assay against the currently evaluated targets in the EGFP coding sequence. These data clearly demonstrate that several of the selected Type V-A Cas proteins were able to modify very efficiently genetic targets in mammalian cells and can thus be exploited to edit the mammalian genome.

### 7.2.4. Evaluation of ZZKD Type V-A Cas Protein Editing Activity on Benchmark Genomic Loci in Mammalian Cells

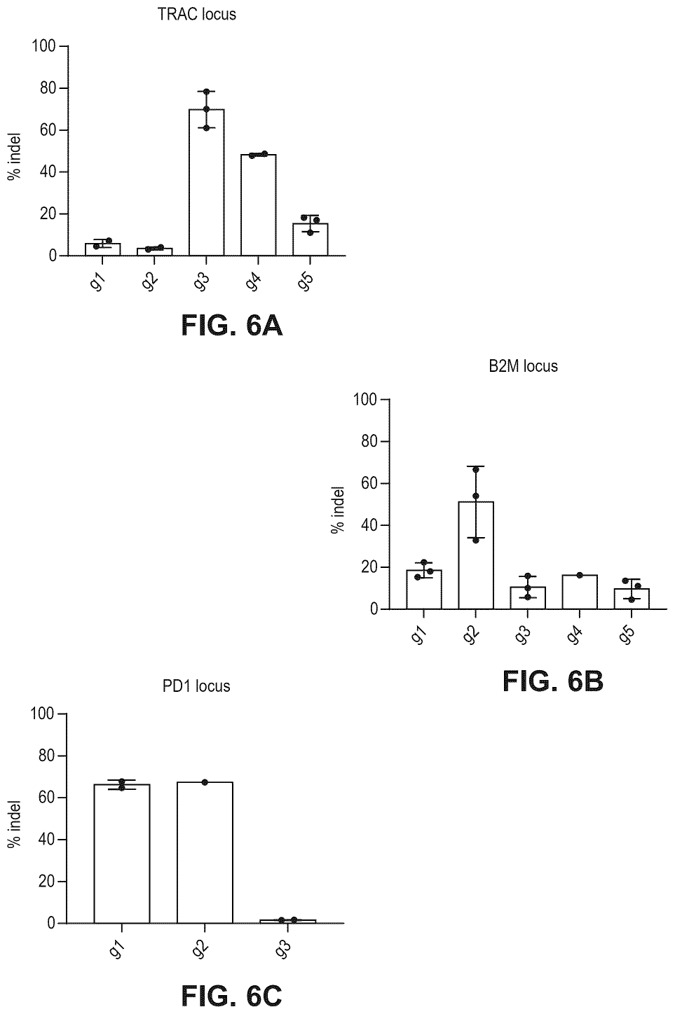

To further validate the editing activity of the highest performing candidate Type V-A Cas protein in the EGFP assay, ZZKD, guide RNAs were designed to target the B2M, TRAC and PD1 benchmark genomic loci in human cells. U2OS cells were electroporated with plasmids encoding ZZKD Type V-A Cas and the selected gRNAs and indel formation was measured by Sanger chromatogram deconvolution on extracted genomic DNA. Strikingly, for all three target loci it was possible to identify at least one gRNA showing high levels of genomic modification (>40%, see FIG. 6A-C) and except for the B2M target locus more than one well performing guide was identified (g3-g4 for the TRAC locus, g1-g2 for the PD1 locus).

Overall these data clearly demonstrate that ZZKD Type V-A is proficient in editing the human genome at several target sites.

### 7.3. Example 2: Further Characterization of Novel Type V-A Cas Molecules

This Example describes studies performed to further characterize Type V-A Cas proteins identified in Example 1.

### 7.3.1. Evaluation of Additional Type V-A Cas Proteins Editing Activity Using an EGFP Reporter System

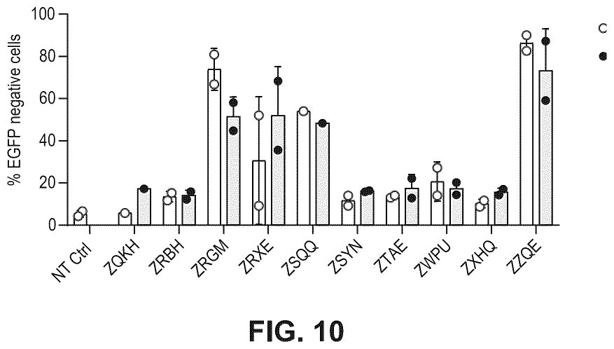

Leveraging on the conserved nature of PAM preferences among Type V-A Cas proteins, guide RNAs targeting the EGFP coding sequence were designed for novel Type V-A Cas proteins isolated from the human microbiome to evaluate their activity in human cells. An EGFP reporter system was used as it allowed an easier readout on the editing activity, based on the loss of fluorescence of treated cells quantitatively measured by cytofluorimetry. Two gRNAs targeting the EGFP coding sequence were designed and evaluated in U2OS cells stably expressing a single copy of the EGFP reporter by transient electroporation. As reported in FIG. 10, while most of the evaluated nucleases showed relatively low levels of EGFP downregulation in this particular assay (close to the detection limit of the assay), some of the selected enzymes were particularly proficient in editing their target sequence. In particular, ZZQE Type V-A Cas protein showed very high activity with both of the evaluated guides (>60% EGFP KO), followed by ZRGM, ZSQQ and ZRXE Type V-A Cas proteins which showed appreciable knock-out activity (>40% EGFP KO) with at least one of the evaluated gRNAs. These data clearly demonstrate that several of the selected Type V-A Cas proteins were able to very efficiently modify genetic targets in mammalian cells and can thus be exploited to modify the genome of mammalian cells.

### 7.3.2. Evaluation of Novel Type V-A Cas Proteins Editing Activity on Benchmark Genomic Loci in Mammalian Cells

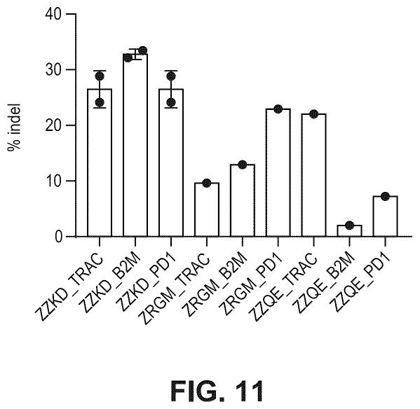

The evaluation of the editing activity of the top performing Type V-A Cas proteins from the EGFP reporter assay KO, ZZKD, ZRGM and ZZQE, was extended to endogenous genomic loci. Guide RNAs were designed to target the B2M (g2), TRAC (g3) and PD1 (g2) benchmark genomic loci in human cells. HEK293T cells were lipofected with plasmids encoding ZZKD, ZRGM and ZZQE Type V-A Cas proteins and the selected gRNAs and indel formation was measured by Sanger chromatogram deconvolution on extracted genomic DNA. Strikingly, for all three target loci all evaluated Type V-A Cas proteins were able to produce appreciable levels of indels, with some variability depending on the target (FIG. 11).

Overall, these data clearly demonstrate that among the selected Type V-A Cas proteins, ZZKD is the most efficient in editing the human genome at several target sites.

### 7.3.3. In Vitro Determination of the PAM Specificity of Top-Performing Novel Type V-A Cas Proteins

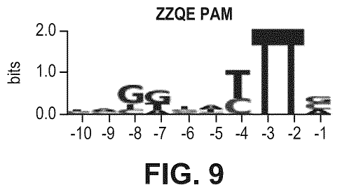

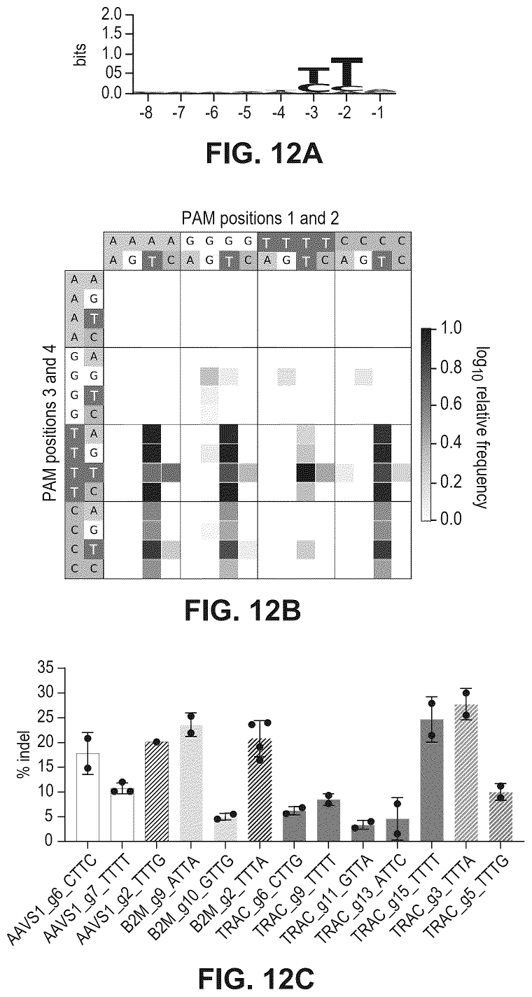

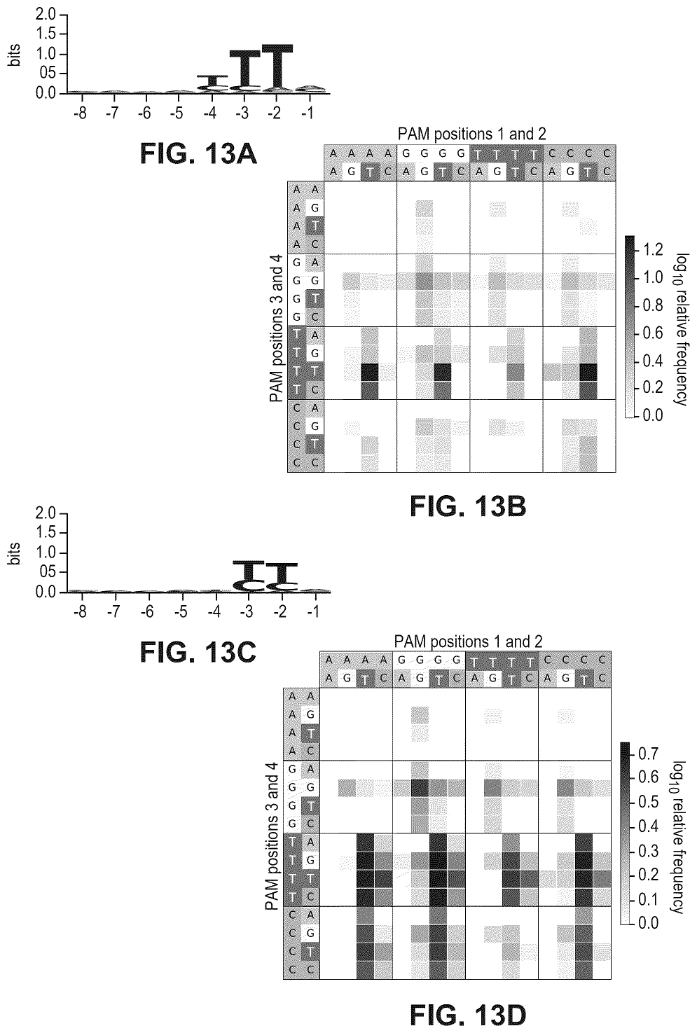

After a first evaluation of their activity in mammalian cells, the PAM preferences of the top performing Type V-A Cas proteins were determined using a well-established in vitro assay. Briefly, ZZKD, ZRGM and ZZQE Type V-A Cas proteins were expressed in HEK293T cells to generate cell lysates which were then used in an in vitro cleavage reaction where a plasmid library including a known target flanked by a randomized 8 nt sequence was cut based on PAM recognition preferences by ribonucleoprotein complexes generated using the cell-expressed nucleases and an in vitro transcribed gRNA targeting the library. Cleaved plasmids were then recovered by amplification and sequenced to determine which PAM sequences were preferentially cleaved (see Materials and Methods for more details). These results confirmed the predicted PAM preferences for ZZKD and ZZQE (see FIG. 4C and FIG. 9, respectively), and in general confirmed the possibility to recognize the TTTV PAM which was used in the initial editing evaluation studies, but showed also additional recognition capabilities. The PAM logos and heatmaps for all the selected Type V-A Cas proteins are reported in FIG. 12A-12B, and FIGS. 13A-13D, while a summary of the in vitro determined PAMs are included in Table 14.

TABLE 14
In vitro determined PAM sequences for selected
Type V-A Cas proteins

| Type V-A Cas protein | PAM (5′→3′) |
|---|---|
| ZZKD Type V-A Cas | NTTV, VTTV, NCTV, TTTT |
| ZRGM Type V-A Cas | YTTV |
| ZZQE Type V-A Cas | NYYN, NTTN, NCTV |

To further confirm the PAM preferences determined for ZZKD Type V-A Cas, a panel of guide RNAs targeting loci flanked by a VTTV and TTTT PAMs was selected and the editing efficacy of ZZKD towards these loci was evaluated after transient transfection in HEK293T cells. As shown in FIG. 12C, many of the evaluated guides showed efficient editing of the target locus demonstrating the possibility for ZZKD to recognize such PAMs, as indicated by the in vitro assay.

### 7.4. Example 3: Further Characterization of ZZKD Type V-A Cas Protein

This example describes additional studies to characterize ZZKD Type V-A Cas protein.

### 7.4.1. Evaluation of the Cleavage Profile of ZZKD Type V-A Cas Protein

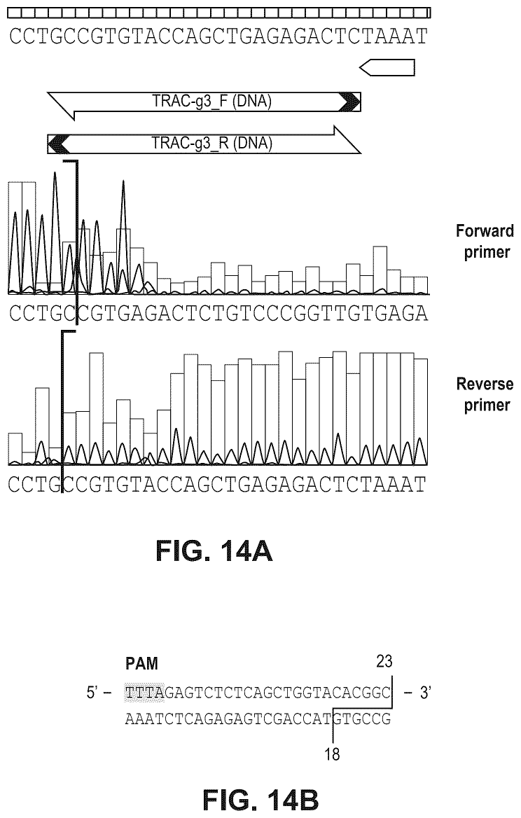

To further characterize the enzymatic activity of ZZKD Type V-A Cas protein, its cleavage profile was investigated in vitro. Recombinant ZZKD was used to digest in vitro a dsDNA target obtained by PCR amplification of a known target region (TRAC locus, g3). The digestion products were separated on agarose gel and independently Sanger sequenced. Based on the two chromatographic profiles (FIG. 14A), it was possible to determine where the two DNA strands were cut: a staggered double strand break was produced, with the non-target strand cut 23nt downstream (5′>3′) of the PAM and the target strand cut 18nt upstream (5′>3′). This is in line with what was observed for other well characterized Type V-A Cas proteins.

### 7.4.2. Evaluation of Different Nuclear Localization Signals (NLS) for ZZKD Type V-A Cas Protein

In order to further improve the editing activity of the ZZKD Type V-A Cas protein, alternative types and positioning of nuclear localization signals were evaluated. The amino acid sequence of the different NLS evaluated as well as the relative position are indicated in Table 15 below.

TABLE 15
Nuclear localization signals evaluated in the example

| Name | Position | Amino acid sequence | SEQ ID |
|---|---|---|---|
| SV40 | N-term | PKKKRKVG | 179 |
| bpNLS | C-term | KRTADGSEFESPKKKRKV | 122 |
| FL-SV40 | C-term | GRSSDDEATADSQHAAPPKKKRKV | 180 |
| npNLS | C-term | KRPAATKKAGQAKKKK | 125 |

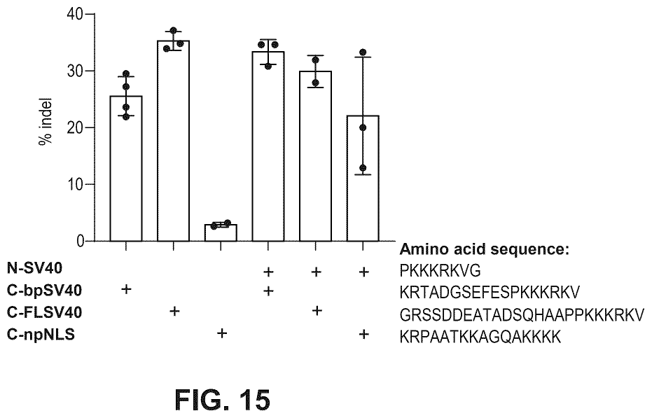

As shown in FIG. 15, when the effect on editing activity of the different NLS designs was evaluated by transient transfection in HEK293T cells using the TRAC benchmark locus (g3) as a target, most of the constructs showed high editing levels with the exception of the single npNLS at the C-terminus, as indicated on the graph. Among all evaluated constructs, the FL-SV40 C-term performed particularly well and was thus used in subsequent studies.

### 7.5. Example 4: Novel Type V-A Cas Protein Alternative crRNA Scaffolds

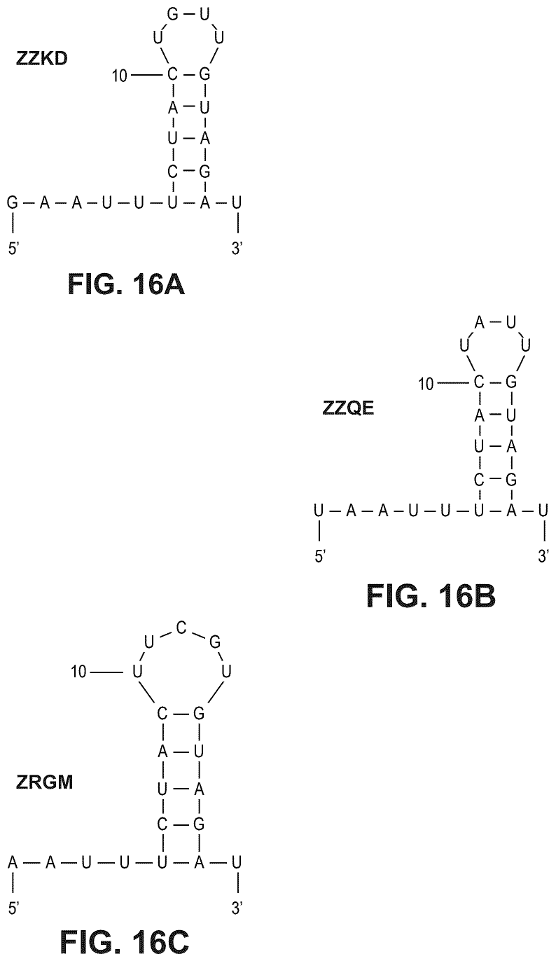

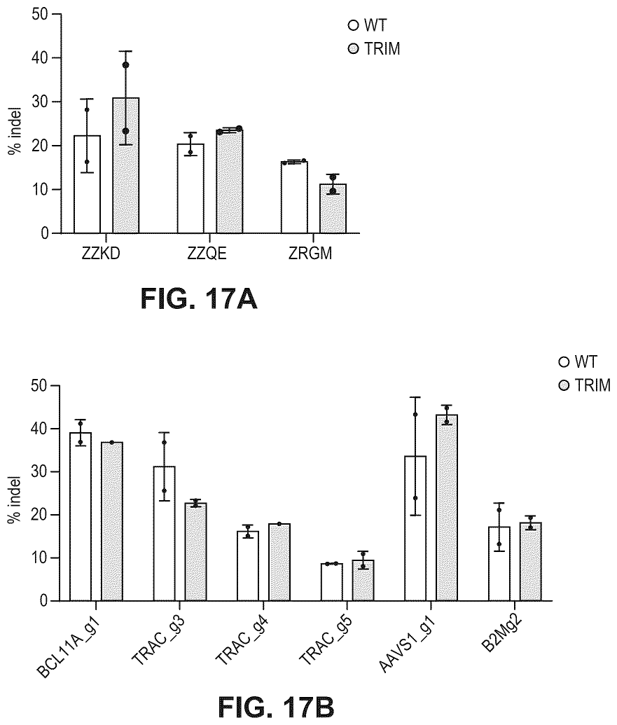

Alternative trimmed scaffolds were evaluated for the top performing identified Type V-A Cas proteins (ZZKD, ZRGM, ZZQE). The editing activity of these enzymes was evaluated using the standard full length scaffold (36 nt) in comparison to a shorter 20nt scaffold, which nevertheless preserves a conserved stem-loop structure shared among the different crRNAs (FIGS. 16A-16C), using the TRAC locus (g3) as a benchmark. After transient transfection in HEK293T cells, indels were measured at the target locus revealing similar editing levels with both versions of the crRNA for all the evaluated nucleases (FIG. 17A). To further confirm this finding, ZZKD Type V-A Cas protein was evaluated on an extended panel of loci including additional guides on TRAC, BCL11A, AAVS1 and B2M. These studies confirmed a similar activity for both versions of the scaffold (FIG. 17B), in accordance with previously generated data. Overall, this demonstrates that truncating the 5′-end of the crRNA scaffold does not negatively influence the editing activity of these Type V-A Cas proteins after transfection in human cells.

### 7.6. Example 5: Evaluation of ZZKD Type V-A Cas Protein Spacer Length

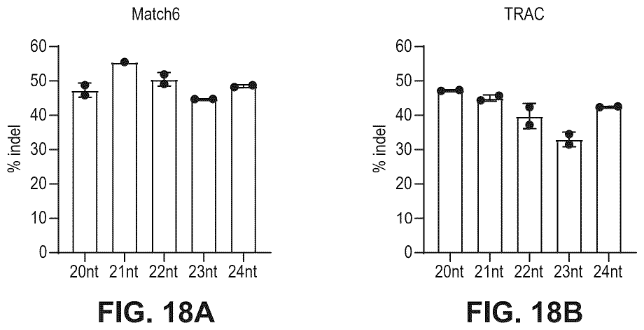

With the aim of further improving the editing activity of ZZKD Type V-A Cas, different spacer lengths were evaluated to determine which favored the highest target modification. crRNAs with spacer lengths ranging from 20nt to 24nt were evaluated by targeting the TRAC (g3) and Match6 (see, Kleinstiver et al., 2016, Nat Biotechnol. 34 (8): 869-74) benchmark loci by transient transfection in HEK293T cells. While appreciable editing levels were observed for all the evaluated lengths (FIGS. 18A-18B), shorter spacers were generally offering higher activity, with 21nt being the most preferred length.

### 7.7. Example 6: Side-by-Side Comparison of ZZKD Type V-A Cas Protein Activity with the Commercially Available Benchmark AsCas12a Ultra

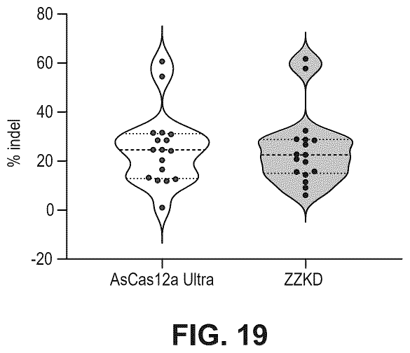

To characterize in depth the editing activity of ZZKD Type V-A Cas, indel formation was compared to the commercially available benchmark AsCas12a Ultra (Zhang et al., 2021, Nat. comms. 12:3908), on a panel of endogenous loci in HEK293T cells after transient transfection. A total of 17 crRNAs targeting multiple genomic loci (TRAC, PD1, EMX1, AAVS1, BCL11A, PCSK9, Match6, VEGFA) were evaluated. Notably, given the PAM compatibility between ZZKD and AsCas12a Ultra, the crRNAs were fully overlapping in all cases. As shown by the violin plots of FIG. 19, summarizing the editing data, the performance of the two nucleases was generally comparable, with ZZKD outperforming AsCas12a Ultra at some loci. The editing levels for each target site for the two nucleases are reported in Table 16 below.

TABLE 16
Editing levels on endogenous target loci after transient
transfection of HEK293T cells (mean ± SD)

| Target site | ZZKD Type V-A Cas | AsCas12a Ultra |
|---|---|---|
| B2M_g2 | 16.50 ± 0.99 | 22.45 ± 3.3 |
| TRAC_g3 | 28.45 ± 1.77 | 28.35 ± 1.6 |
| PD1_g2 | 28.45 ± 1.22 | 26.45 ± 3.3 |
| BCL11A_g1 | 30.85 ± 0.35 | 26.65 ± 1.1 |
| BCL11A_g2 | 24.10 ± 2.12 | 22.7 ± 0.3 |
| BCL11A_g3 | 12.05 ± 3.04 | 19.55 ± 1.1 |
| PCSK9_g1 | 24.60 ± 4.24 | 11.4 ± 0.1 |
| PCSK9_g3 | 13.20 ± 4.95 | 15.7 ± 1.6 |
| AAVS1_g1 | 12.60 ± 0.71 | 15.5 ± 5.7 |
| AAVS1_g2 | 31.55 ± 1.20 | 20.7 ± 0.8 |
| AAVS1_g3 | 11.85 ± 0.07 | 9.05 ± 0.1 |
| Match6 | 28.70 ± 0.28 | 28.65 ± 2.5 |
| BCL11A_g4 | 60.65 ± 8.27 | 57.65 ± 3.5 |
| VEGFA_g1 | 33.75 ± 3.18 | 32.35 ± 0.6 |
| EMX1_g2 | 0.95 ± 0.78 | 6 ± 0.4 |
| EMX1_g3 | 20.35 ± 0.35 | 14.35 ± 3.5 |
| B2M_g1_21nt | 54.50 ± 9.19 | 61.6 |

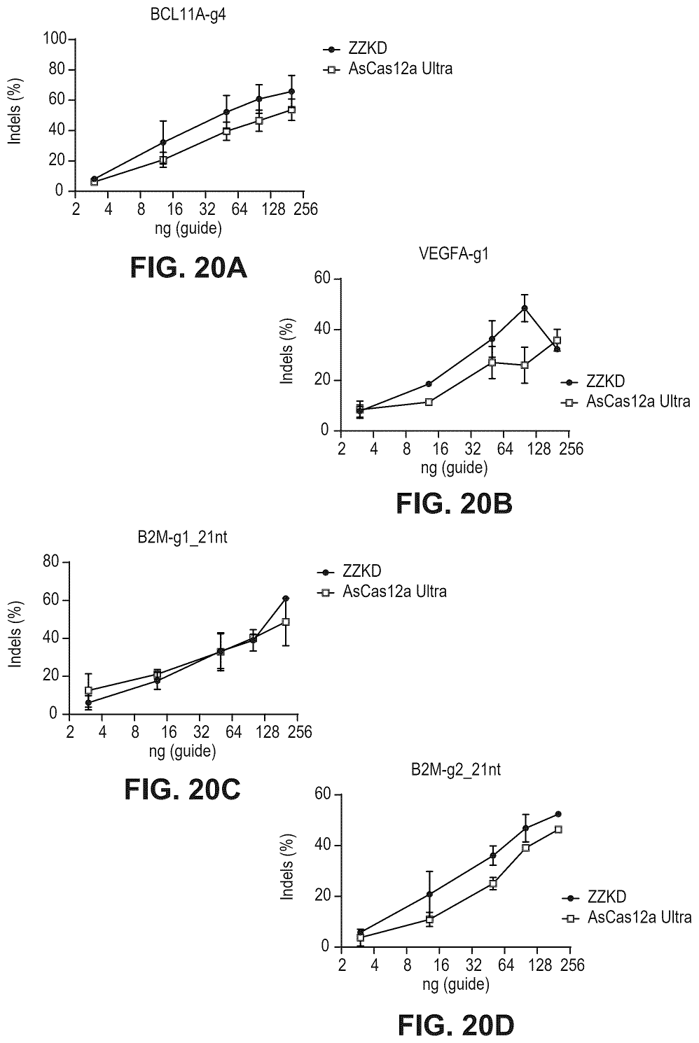

Further to these editing studies, titration studies, where the amounts of transfected nuclease and guide RNA are progressively lowered to better measure differences in the editing activity, were performed on a selection of target loci (BCL11A-g4, B2M-g1 and B2M-g2, VEGFA-g1) in HEK293T cells. As shown in FIGS. 20A-20D, all titration curves showed generally comparable editing activities of the two proteins, with the general tendency for ZZKD Type V-A Cas to perform better than the AsCas12a Ultra benchmark (see for example VEGFA-g1 in FIG. 20B).

Overall, these data demonstrate that ZZKD Type V-A Cas protein is able to match or outperform the editing activity of the current state-of-the-art commercial AsCas12 Ultra benchmark.

### 7.8. Example 7: Type V-A Cas Protein Activity after Direct Protein Delivery in Cells

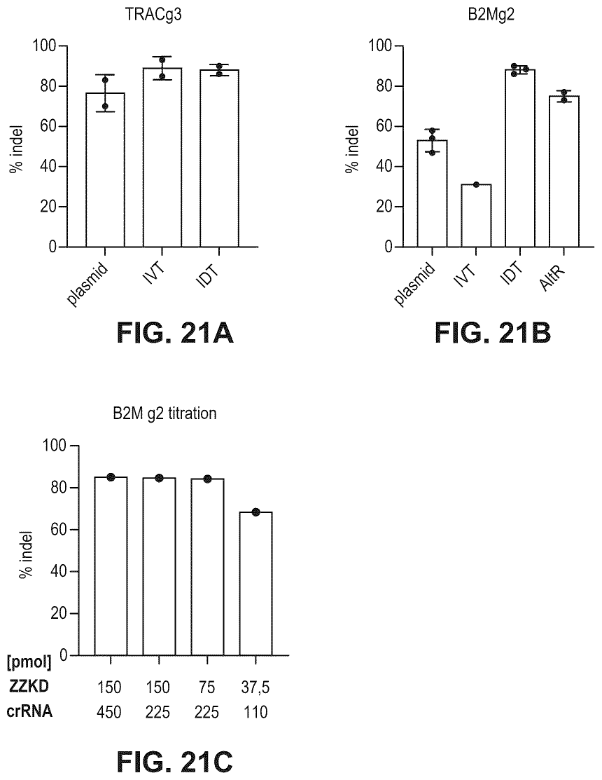

To demonstrate the efficacy of ZZKD Type V-A Cas protein using alternative delivery modalities, direct ribonucleoprotein (RNP) complex delivery to target cells by electroporation was performed. To this aim, recombinant ZZKD was produced in bacteria and was purified by multiple rounds of chromatography using standard techniques, while crRNAs were obtained either from IDT (chemical synthesis) or through in vitro transcription using the T7 RNA polymerase. The activity of the RNP was initially evaluated in U2OS cells using guides targeting the TRAC (FIG. 21A) and B2M (FIG. 21B) loci. The observed editing activity was generally higher than that of the corresponding electroporated plasmid and, among the different types of crRNA evaluated, the synthetic crRNAs performed better. An AltR-modified guide (a chemical modification available from IDT) targeting B2M was also included in the panel showing editing levels close to the unmodified synthetic guide. Additionally, a titration study using B2M-g2 crRNA was performed by lowering progressively the amount of either recombinant ZZKD or corresponding crRNA and also changing the protein: crRNA ratio from 1:3 to 1:1.5 in order to more stringently evaluate ZZKD potency. As shown in FIG. 21C, in most of the conditions evaluated ZZKD Type V-A Cas protein preserved high levels of editing activity indicating high potency even at low concentrations.

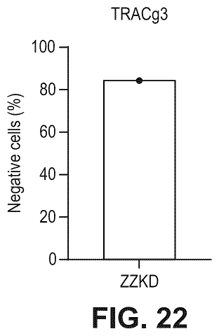

To further confirm the activity of ZZKD Type V-A Cas as RNP, human commercial primary T cells were electroporated with the complex including a guide targeting the TRAC locus (g3). As shown in FIG. 22, ZZKD was able to produce approximately 80% of TRAC-negative cells as measured by cytofluorimetry, demonstrating high editing activity.

Overall, these data show not only that ZZKD Type V-A Cas protein is compatible with direct protein delivery in multiple cell types including hard-to-edit primary T cells but that ZZKD is also highly potent and can be used at low concentrations to obtain efficient target modification.

### 8. SPECIFIC EMBODIMENTS

The present disclosure is exemplified by the specific embodiments below.

1. A Type V Cas protein comprising an amino acid sequence having at least 50% sequence identity to:
(a) the amino acid sequence of a WED-1 domain of a reference protein sequence;
(b) the amino acid sequence of a REC1 domain of a reference protein sequence;
(c) the amino acid sequence of a REC2 domain of a reference protein sequence;
(d) the amino acid sequence of a WED-II domain of a reference protein sequence;
(e) the amino acid sequence of a PI domain of a reference protein sequence;
(f) the amino acid sequence of a WED-III domain of a reference protein sequence;
(g) the amino acid sequence of a RuvC-I domain of a reference protein sequence;
(h) the amino acid sequence of a BH domain of a reference protein sequence;
(i) the amino acid sequence of a RuvC-II domain of a reference protein sequence;
(j) the amino acid sequence of a NUC domain of a reference protein sequence;
(k) the amino acid sequence of a RuvC-III domain of a reference protein sequence; or
(l) the amino acid sequence of the full length of a reference protein sequence;
wherein the reference protein sequence is SEQ ID NO:1, SEQ ID NO:2, SEQ ID NO:7, SEQ ID NO: 8, SEQ ID NO: 13, SEQ ID NO: 14, SEQ ID NO: 19, SEQ ID NO:20, SEQ ID NO:25, SEQ ID NO:26, SEQ ID NO:31, SEQ ID NO: 32, SEQ ID NO:37, SEQ ID NO:38, SEQ ID NO:43, SEQ ID NO:44, SEQ ID NO: 49, SEQ ID NO: 50, SEQ ID NO:55, SEQ ID NO:56, SEQ ID NO:61, SEQ ID NO:62, SEQ ID NO:67, SEQ ID NO: 68, SEQ ID NO: 73, SEQ ID NO: 74, SEQ ID NO: 79, SEQ ID NO: 80, SEQ ID NO: 85, SEQ ID NO: 86, SEQ ID NO: 91, SEQ ID NO: 92, SEQ ID NO:97, SEQ ID NO:98, SEQ ID NO: 103, SEQ ID NO: 104, SEQ ID NO: 109, SEQ ID NO: 110, SEQ ID NO: 115, or SEQ ID NO: 116.

2. The Type V Cas protein of embodiment 1, wherein the amino acid sequence of the Type V Cas protein comprises an amino acid sequence that is at least 50% identical to the amino acid sequence of the WED-I domain of the reference protein sequence.

3. The Type V Cas protein of embodiment 1, wherein the amino acid sequence of the Type V Cas protein comprises an amino acid sequence that is at least 55% identical to the amino acid sequence of the WED-I domain of the reference protein sequence.

4. The Type V Cas protein of embodiment 1, wherein the amino acid sequence of the Type V Cas protein comprises an amino acid sequence that is at least 60% identical to the amino acid sequence of the WED-I domain of the reference protein sequence.

5. The Type V Cas protein of embodiment 1, wherein the amino acid sequence of the Type V Cas protein comprises an amino acid sequence that is at least 65% identical to the amino acid sequence of the WED-I domain of the reference protein sequence.

6. The Type V Cas protein of embodiment 1, wherein the amino acid sequence of the Type V Cas protein comprises an amino acid sequence that is at least 70% identical to the amino acid sequence of the WED-I domain of the reference protein sequence.

7. The Type V Cas protein of embodiment 1, wherein the amino acid sequence of the Type V Cas protein comprises an amino acid sequence that is at least 75% identical to the amino acid sequence of the WED-I domain of the reference protein sequence.

8. The Type V Cas protein of embodiment 1, wherein the amino acid sequence of the Type V Cas protein comprises an amino acid sequence that is at least 80% identical to the amino acid sequence of the WED-I domain of the reference protein sequence.

9. The Type V Cas protein of embodiment 1, wherein the amino acid sequence of the Type V Cas protein comprises an amino acid sequence that is at least 85% identical to the amino acid sequence of the WED-I domain of the reference protein sequence.

10. The Type V Cas protein of embodiment 1, wherein the amino acid sequence of the Type V Cas protein comprises an amino acid sequence that is at least 90% identical to the amino acid sequence of the WED-I domain of the reference protein sequence.

11. The Type V Cas protein of embodiment 1, wherein the amino acid sequence of the Type V Cas protein comprises an amino acid sequence that is at least 95% identical to the amino acid sequence of the WED-I domain of the reference protein sequence.

12. The Type V Cas protein of embodiment 1, wherein the amino acid sequence of the Type V Cas protein comprises an amino acid sequence that is at least 96% identical to the amino acid sequence of the WED-I domain of the reference protein sequence.

13. The Type V Cas protein of embodiment 1, wherein the amino acid sequence of the Type V Cas protein comprises an amino acid sequence that is at least 97% identical to the amino acid sequence of the WED-I domain of the reference protein sequence.

14. The Type V Cas protein of embodiment 1, wherein the amino acid sequence of the Type V Cas protein comprises an amino acid sequence that is at least 98% identical to the amino acid sequence of the WED-I domain of the reference protein sequence.

15. The Type V Cas protein of embodiment 1, wherein the amino acid sequence of the Type V Cas protein comprises an amino acid sequence that is at least 99% identical to the amino acid sequence of the WED-I domain of the reference protein sequence.

16. The Type V Cas protein of embodiment 1, wherein the amino acid sequence of the Type V Cas protein comprises an amino acid sequence that is identical to the amino acid sequence of the WED-I domain of the reference protein sequence.

17. The Type V Cas protein of any one of embodiments 1 to 16, wherein the amino acid sequence of the Type V Cas protein comprises an amino acid sequence that is at least 50% identical to the amino acid sequence of the REC1 domain of the reference protein sequence.

18. The Type V Cas protein of any one of embodiments 1 to 16, wherein the amino acid sequence of the Type V Cas protein comprises an amino acid sequence that is at least 55% identical to the amino acid sequence of the REC1 domain of the reference protein sequence.

19. The Type V Cas protein of any one of embodiments 1 to 16, wherein the amino acid sequence of the Type V Cas protein comprises an amino acid sequence that is at least 60% identical to the amino acid sequence of the REC1 domain of the reference protein sequence.

20. The Type V Cas protein of any one of embodiments 1 to 16, wherein the amino acid sequence of the Type V Cas protein comprises an amino acid sequence that is at least 65% identical to the amino acid sequence of the REC1 domain of the reference protein sequence.

21. The Type V Cas protein of any one of embodiments 1 to 16, wherein the amino acid sequence of the Type V Cas protein comprises an amino acid sequence that is at least 70% identical to the amino acid sequence of the REC1 domain of the reference protein sequence.

22. The Type V Cas protein of any one of embodiments 1 to 16, wherein the amino acid sequence of the Type V Cas protein comprises an amino acid sequence that is at least 75% identical to the amino acid sequence of the REC1 domain of the reference protein sequence.

23. The Type V Cas protein of any one of embodiments 1 to 16, wherein the amino acid sequence of the Type V Cas protein comprises an amino acid sequence that is at least 80% identical to the amino acid sequence of the REC1 domain of the reference protein sequence.

24. The Type V Cas protein of any one of embodiments 1 to 16, wherein the amino acid sequence of the Type V Cas protein comprises an amino acid sequence that is at least 85% identical to the amino acid sequence of the REC1 domain of the reference protein sequence.

25. The Type V Cas protein of any one of embodiments 1 to 16, wherein the amino acid sequence of the Type V Cas protein comprises an amino acid sequence that is at least 90% identical to the amino acid sequence of the REC1 domain of the reference protein sequence.

26. The Type V Cas protein of any one of embodiments 1 to 16, wherein the amino acid sequence of the Type V Cas protein comprises an amino acid sequence that is at least 95% identical to the amino acid sequence of the REC1 domain of the reference protein sequence.

27. The Type V Cas protein of any one of embodiments 1 to 16, wherein the amino acid sequence of the Type V Cas protein comprises an amino acid sequence that is at least 96% identical to the amino acid sequence of the REC1 domain of the reference protein sequence.

28. The Type V Cas protein of any one of embodiments 1 to 16, wherein the amino acid sequence of the Type V Cas protein comprises an amino acid sequence that is at least 97% identical to the amino acid sequence of the REC1 domain of the reference protein sequence.

29. The Type V Cas protein of any one of embodiments 1 to 16, wherein the amino acid sequence of the Type V Cas protein comprises an amino acid sequence that is at least 98% identical to the amino acid sequence of the REC1 domain of the reference protein sequence.

30. The Type V Cas protein of any one of embodiments 1 to 16, wherein the amino acid sequence of the Type V Cas protein comprises an amino acid sequence that is at least 99% identical to the amino acid sequence of the REC1 domain of the reference protein sequence.

31. The Type V Cas protein of any one of embodiments 1 to 16, wherein the amino acid sequence of the Type V Cas protein comprises an amino acid sequence that is identical to the amino acid sequence of the REC1 domain of the reference protein sequence.

32. The Type V Cas protein of any one of embodiments 1 to 31, wherein the amino acid sequence of the Type V Cas protein comprises an amino acid sequence that is at least 50% identical to the amino acid sequence of the REC2 domain of the reference protein sequence.

33. The Type V Cas protein of any one of embodiments 1 to 31, wherein the amino acid sequence of the Type V Cas protein comprises an amino acid sequence that is at least 55% identical to the amino acid sequence of the REC2 domain of the reference protein sequence.

34. The Type V Cas protein of any one of embodiments 1 to 31, wherein the amino acid sequence of the Type V Cas protein comprises an amino acid sequence that is at least 60% identical to the amino acid sequence of the REC2 domain of the reference protein sequence.

35. The Type V Cas protein of any one of embodiments 1 to 31, wherein the amino acid sequence of the Type V Cas protein comprises an amino acid sequence that is at least 65% identical to the amino acid sequence of the REC2 domain of the reference protein sequence.

36. The Type V Cas protein of any one of embodiments 1 to 31, wherein the amino acid sequence of the Type V Cas protein comprises an amino acid sequence that is at least 70% identical to the amino acid sequence of the REC2 domain of the reference protein sequence.

37. The Type V Cas protein of any one of embodiments 1 to 31, wherein the amino acid sequence of the Type V Cas protein comprises an amino acid sequence that is at least 75% identical to the amino acid sequence of the REC2 domain of the reference protein sequence.

38. The Type V Cas protein of any one of embodiments 1 to 31, wherein the amino acid sequence of the Type V Cas protein comprises an amino acid sequence that is at least 80% identical to the amino acid sequence of the REC2 domain of the reference protein sequence.

39. The Type V Cas protein of any one of embodiments 1 to 31, wherein the amino acid sequence of the Type V Cas protein comprises an amino acid sequence that is at least 85% identical to the amino acid sequence of the REC2 domain of the reference protein sequence.

40. The Type V Cas protein of any one of embodiments 1 to 31, wherein the amino acid sequence of the Type V Cas protein comprises an amino acid sequence that is at least 90% identical to the amino acid sequence of the REC2 domain of the reference protein sequence.

41. The Type V Cas protein of any one of embodiments 1 to 31, wherein the amino acid sequence of the Type V Cas protein comprises an amino acid sequence that is at least 95% identical to the amino acid sequence of the REC2 domain of the reference protein sequence.

42. The Type V Cas protein of any one of embodiments 1 to 31, wherein the amino acid sequence of the Type V Cas protein comprises an amino acid sequence that is at least 96% identical to the amino acid sequence of the REC2 domain of the reference protein sequence.

43. The Type V Cas protein of any one of embodiments 1 to 31, wherein the amino acid sequence of the Type V Cas protein comprises an amino acid sequence that is at least 97% identical to the amino acid sequence of the REC2 domain of the reference protein sequence.

44. The Type V Cas protein of any one of embodiments 1 to 31, wherein the amino acid sequence of the Type V Cas protein comprises an amino acid sequence that is at least 98% identical to the amino acid sequence of the REC2 domain of the reference protein sequence.

45. The Type V Cas protein of any one of embodiments 1 to 31, wherein the amino acid sequence of the Type V Cas protein comprises an amino acid sequence that is at least 99% identical to the amino acid sequence of the REC2 domain of the reference protein sequence.

46. The Type V Cas protein of any one of embodiments 1 to 31, wherein the amino acid sequence of the Type V Cas protein comprises an amino acid sequence that is identical to the amino acid sequence of the REC2 domain of the reference protein sequence.

47. The Type V Cas protein of any one of embodiments 1 to 46, wherein the amino acid sequence of the Type V Cas protein comprises an amino acid sequence that is at least 50% identical to the amino acid sequence of the WED-II domain of the reference protein sequence.

48. The Type V Cas protein of any one of embodiments 1 to 46, wherein the amino acid sequence of the Type V Cas protein comprises an amino acid sequence that is at least 55% identical to the amino acid sequence of the WED-II domain of the reference protein sequence.

49. The Type V Cas protein of any one of embodiments 1 to 46, wherein the amino acid sequence of the Type V Cas protein comprises an amino acid sequence that is at least 60% identical to the amino acid sequence of the WED-II domain of the reference protein sequence.

50. The Type V Cas protein of any one of embodiments 1 to 46, wherein the amino acid sequence of the Type V Cas protein comprises an amino acid sequence that is at least 65% identical to the amino acid sequence of the WED-II domain of the reference protein sequence.

51. The Type V Cas protein of any one of embodiments 1 to 46, wherein the amino acid sequence of the Type V Cas protein comprises an amino acid sequence that is at least 70% identical to the amino acid sequence of the WED-II domain of the reference protein sequence.

52. The Type V Cas protein of any one of embodiments 1 to 46, wherein the amino acid sequence of the Type V Cas protein comprises an amino acid sequence that is at least 75% identical to the amino acid sequence of the WED-II domain of the reference protein sequence.

53. The Type V Cas protein of any one of embodiments 1 to 46, wherein the amino acid sequence of the Type V Cas protein comprises an amino acid sequence that is at least 80% identical to the amino acid sequence of the WED-II domain of the reference protein sequence.

54. The Type V Cas protein of any one of embodiments 1 to 46, wherein the amino acid sequence of the Type V Cas protein comprises an amino acid sequence that is at least 85% identical to the amino acid sequence of the WED-II domain of the reference protein sequence.

55. The Type V Cas protein of any one of embodiments 1 to 46, wherein the amino acid sequence of the Type V Cas protein comprises an amino acid sequence that is at least 90% identical to the amino acid sequence of the WED-II domain of the reference protein sequence.

56. The Type V Cas protein of any one of embodiments 1 to 46, wherein the amino acid sequence of the Type V Cas protein comprises an amino acid sequence that is at least 95% identical to the amino acid sequence of the WED-II domain of the reference protein sequence.

57. The Type V Cas protein of any one of embodiments 1 to 46, wherein the amino acid sequence of the Type V Cas protein comprises an amino acid sequence that is at least 96% identical to the amino acid sequence of the WED-II domain of the reference protein sequence.

58. The Type V Cas protein of any one of embodiments 1 to 46, wherein the amino acid sequence of the Type V Cas protein comprises an amino acid sequence that is at least 97% identical to the amino acid sequence of the WED-II domain of the reference protein sequence.

59. The Type V Cas protein of any one of embodiments 1 to 46, wherein the amino acid sequence of the Type V Cas protein comprises an amino acid sequence that is at least 98% identical to the amino acid sequence of the WED-II domain of the reference protein sequence.

60. The Type V Cas protein of any one of embodiments 1 to 46, wherein the amino acid sequence of the Type V Cas protein comprises an amino acid sequence that is at least 99% identical to the amino acid sequence of the WED-II domain of the reference protein sequence.

61. The Type V Cas protein of any one of embodiments 1 to 46, wherein the amino acid sequence of the Type V Cas protein comprises an amino acid sequence that is identical to the amino acid sequence of the WED-II domain of the reference protein sequence.

62. The Type V Cas protein of any one of embodiments 1 to 61, wherein the amino acid sequence of the Type V Cas protein comprises an amino acid sequence that is at least 50% identical to the amino acid sequence of the PI domain of the reference protein sequence.

63. The Type V Cas protein of any one of embodiments 1 to 61, wherein the amino acid sequence of the Type V Cas protein comprises an amino acid sequence that is at least 55% identical to the amino acid sequence of the PI domain of the reference protein sequence.

64. The Type V Cas protein of any one of embodiments 1 to 61, wherein the amino acid sequence of the Type V Cas protein comprises an amino acid sequence that is at least 60% identical to the amino acid sequence of the PI domain of the reference protein sequence.

65. The Type V Cas protein of any one of embodiments 1 to 61, wherein the amino acid sequence of the Type V Cas protein comprises an amino acid sequence that is at least 65% identical to the amino acid sequence of the PI domain of the reference protein sequence.

66. The Type V Cas protein of any one of embodiments 1 to 61, wherein the amino acid sequence of the Type V Cas protein comprises an amino acid sequence that is at least 70% identical to the amino acid sequence of the PI domain of the reference protein sequence.

67. The Type V Cas protein of any one of embodiments 1 to 61, wherein the amino acid sequence of the Type V Cas protein comprises an amino acid sequence that is at least 75% identical to the amino acid sequence of the PI domain of the reference protein sequence.

68. The Type V Cas protein of any one of embodiments 1 to 61, wherein the amino acid sequence of the Type V Cas protein comprises an amino acid sequence that is at least 80% identical to the amino acid sequence of the PI domain of the reference protein sequence.

69. The Type V Cas protein of any one of embodiments 1 to 61, wherein the amino acid sequence of the Type V Cas protein comprises an amino acid sequence that is at least 85% identical to the amino acid sequence of the PI domain of the reference protein sequence.

70. The Type V Cas protein of any one of embodiments 1 to 61, wherein the amino acid sequence of the Type V Cas protein comprises an amino acid sequence that is at least 90% identical to the amino acid sequence of the PI domain of the reference protein sequence.

71. The Type V Cas protein of any one of embodiments 1 to 61, wherein the amino acid sequence of the Type V Cas protein comprises an amino acid sequence that is at least 95% identical to the amino acid sequence of the PI domain of the reference protein sequence.

72. The Type V Cas protein of any one of embodiments 1 to 61, wherein the amino acid sequence of the Type V Cas protein comprises an amino acid sequence that is at least 96% identical to the amino acid sequence of the PI domain of the reference protein sequence.

73. The Type V Cas protein of any one of embodiments 1 to 61, wherein the amino acid sequence of the Type V Cas protein comprises an amino acid sequence that is at least 97% identical to the amino acid sequence of the PI domain of the reference protein sequence.

74. The Type V Cas protein of any one of embodiments 1 to 61, wherein the amino acid sequence of the Type V Cas protein comprises an amino acid sequence that is at least 98% identical to the amino acid sequence of the PI domain of the reference protein sequence.

75. The Type V Cas protein of any one of embodiments 1 to 61, wherein the amino acid sequence of the Type V Cas protein comprises an amino acid sequence that is at least 99% identical to the amino acid sequence of the PI domain of the reference protein sequence.

76. The Type V Cas protein of any one of embodiments 1 to 61, wherein the amino acid sequence of the Type V Cas protein comprises an amino acid sequence that is identical to the amino acid sequence of the PI domain of the reference protein sequence.

77. The Type V Cas protein of any one of embodiments 1 to 76, wherein the amino acid sequence of the Type V Cas protein comprises an amino acid sequence that is at least 50% identical to the amino acid sequence of the WED-III domain of the reference protein sequence.

78. The Type V Cas protein of any one of embodiments 1 to 76, wherein the amino acid sequence of the Type V Cas protein comprises an amino acid sequence that is at least 55% identical to the amino acid sequence of the WED-III domain of the reference protein sequence.

79. The Type V Cas protein of any one of embodiments 1 to 76, wherein the amino acid sequence of the Type V Cas protein comprises an amino acid sequence that is at least 60% identical to the amino acid sequence of the WED-III domain of the reference protein sequence.

80. The Type V Cas protein of any one of embodiments 1 to 76, wherein the amino acid sequence of the Type V Cas protein comprises an amino acid sequence that is at least 65% identical to the amino acid sequence of the WED-III domain of the reference protein sequence.

81. The Type V Cas protein of any one of embodiments 1 to 76, wherein the amino acid sequence of the Type V Cas protein comprises an amino acid sequence that is at least 70% identical to the amino acid sequence of the WED-III domain of the reference protein sequence.

82. The Type V Cas protein of any one of embodiments 1 to 76, wherein the amino acid sequence of the Type V Cas protein comprises an amino acid sequence that is at least 75% identical to the amino acid sequence of the WED-III domain of the reference protein sequence.

83. The Type V Cas protein of any one of embodiments 1 to 76, wherein the amino acid sequence of the Type V Cas protein comprises an amino acid sequence that is at least 80% identical to the amino acid sequence of the WED-III domain of the reference protein sequence.

84. The Type V Cas protein of any one of embodiments 1 to 76, wherein the amino acid sequence of the Type V Cas protein comprises an amino acid sequence that is at least 85% identical to the amino acid sequence of the WED-III domain of the reference protein sequence.

85. The Type V Cas protein of any one of embodiments 1 to 76, wherein the amino acid sequence of the Type V Cas protein comprises an amino acid sequence that is at least 90% identical to the amino acid sequence of the WED-III domain of the reference protein sequence.

86. The Type V Cas protein of any one of embodiments 1 to 76, wherein the amino acid sequence of the Type V Cas protein comprises an amino acid sequence that is at least 95% identical to the amino acid sequence of the WED-III domain of the reference protein sequence.

87. The Type V Cas protein of any one of embodiments 1 to 76, wherein the amino acid sequence of the Type V Cas protein comprises an amino acid sequence that is at least 96% identical to the amino acid sequence of the WED-III domain of the reference protein sequence.

88. The Type V Cas protein of any one of embodiments 1 to 76, wherein the amino acid sequence of the Type V Cas protein comprises an amino acid sequence that is at least 97% identical to the amino acid sequence of the WED-III domain of the reference protein sequence.

89. The Type V Cas protein of any one of embodiments 1 to 76, wherein the amino acid sequence of the Type V Cas protein comprises an amino acid sequence that is at least 98% identical to the amino acid sequence of the WED-III domain of the reference protein sequence.

90. The Type V Cas protein of any one of embodiments 1 to 76, wherein the amino acid sequence of the Type V Cas protein comprises an amino acid sequence that is at least 99% identical to the amino acid sequence of the WED-III domain of the reference protein sequence.

91. The Type V Cas protein of any one of embodiments 1 to 76, wherein the amino acid sequence of the Type V Cas protein comprises an amino acid sequence that is identical to the amino acid sequence of the WED-III domain of the reference protein sequence.

92. The Type V Cas protein of any one of embodiments 1 to 91, wherein the amino acid sequence of the Type V Cas protein comprises an amino acid sequence that is at least 50% identical to the amino acid sequence of the RuvC-I domain of the reference protein sequence.

93. The Type V Cas protein of any one of embodiments 1 to 91, wherein the amino acid sequence of the Type V Cas protein comprises an amino acid sequence that is at least 55% identical to the amino acid sequence of the RuvC-I domain of the reference protein sequence.

94. The Type V Cas protein of any one of embodiments 1 to 91, wherein the amino acid sequence of the Type V Cas protein comprises an amino acid sequence that is at least 60% identical to the amino acid sequence of the RuvC-I domain of the reference protein sequence.

95. The Type V Cas protein of any one of embodiments 1 to 91, wherein the amino acid sequence of the Type V Cas protein comprises an amino acid sequence that is at least 65% identical to the amino acid sequence of the RuvC-I domain of the reference protein sequence.

96. The Type V Cas protein of any one of embodiments 1 to 91, wherein the amino acid sequence of the Type V Cas protein comprises an amino acid sequence that is at least 70% identical to the amino acid sequence of the RuvC-I domain of the reference protein sequence.

97. The Type V Cas protein of any one of embodiments 1 to 91, wherein the amino acid sequence of the Type V Cas protein comprises an amino acid sequence that is at least 75% identical to the amino acid sequence of the RuvC-I domain of the reference protein sequence.

98. The Type V Cas protein of any one of embodiments 1 to 91, wherein the amino acid sequence of the Type V Cas protein comprises an amino acid sequence that is at least 80% identical to the amino acid sequence of the RuvC-I domain of the reference protein sequence.

99. The Type V Cas protein of any one of embodiments 1 to 91, wherein the amino acid sequence of the Type V Cas protein comprises an amino acid sequence that is at least 85% identical to the amino acid sequence of the RuvC-I domain of the reference protein sequence.

100. The Type V Cas protein of any one of embodiments 1 to 91, wherein the amino acid sequence of the Type V Cas protein comprises an amino acid sequence that is at least 90% identical to the amino acid sequence of the RuvC-I domain of the reference protein sequence.

101. The Type V Cas protein of any one of embodiments 1 to 91, wherein the amino acid sequence of the Type V Cas protein comprises an amino acid sequence that is at least 95% identical to the amino acid sequence of the RuvC-I domain of the reference protein sequence.

102. The Type V Cas protein of any one of embodiments 1 to 91, wherein the amino acid sequence of the Type V Cas protein comprises an amino acid sequence that is at least 96% identical to the amino acid sequence of the RuvC-I domain of the reference protein sequence.

103. The Type V Cas protein of any one of embodiments 1 to 91, wherein the amino acid sequence of the Type V Cas protein comprises an amino acid sequence that is at least 97% identical to the amino acid sequence of the RuvC-I domain of the reference protein sequence.

104. The Type V Cas protein of any one of embodiments 1 to 91, wherein the amino acid sequence of the Type V Cas protein comprises an amino acid sequence that is at least 98% identical to the amino acid sequence of the RuvC-I domain of the reference protein sequence.

105. The Type V Cas protein of any one of embodiments 1 to 91, wherein the amino acid sequence of the Type V Cas protein comprises an amino acid sequence that is at least 99% identical to the amino acid sequence of the RuvC-I domain of the reference protein sequence.

106. The Type V Cas protein of any one of embodiments 1 to 91, wherein the amino acid sequence of the Type V Cas protein comprises an amino acid sequence that is identical to the amino acid sequence of the RuvC-I domain of the reference protein sequence.

107. The Type V Cas protein of any one of embodiments 1 to 106, wherein the amino acid sequence of the Type V Cas protein comprises an amino acid sequence that is at least 50% identical to the amino acid sequence of the BH domain of the reference protein sequence.

108. The Type V Cas protein of any one of embodiments 1 to 106, wherein the amino acid sequence of the Type V Cas protein comprises an amino acid sequence that is at least 55% identical to the amino acid sequence of the BH domain of the reference protein sequence.

109. The Type V Cas protein of any one of embodiments 1 to 106, wherein the amino acid sequence of the Type V Cas protein comprises an amino acid sequence that is at least 60% identical to the amino acid sequence of the BH domain of the reference protein sequence.

110. The Type V Cas protein of any one of embodiments 1 to 106, wherein the amino acid sequence of the Type V Cas protein comprises an amino acid sequence that is at least 65% identical to the amino acid sequence of the BH domain of the reference protein sequence.

111. The Type V Cas protein of any one of embodiments 1 to 106, wherein the amino acid sequence of the Type V Cas protein comprises an amino acid sequence that is at least 70% identical to the amino acid sequence of the BH domain of the reference protein sequence.

112. The Type V Cas protein of any one of embodiments 1 to 106, wherein the amino acid sequence of the Type V Cas protein comprises an amino acid sequence that is at least 75% identical to the amino acid sequence of the BH domain of the reference protein sequence.

113. The Type V Cas protein of any one of embodiments 1 to 106, wherein the amino acid sequence of the Type V Cas protein comprises an amino acid sequence that is at least 80% identical to the amino acid sequence of the BH domain of the reference protein sequence.

114. The Type V Cas protein of any one of embodiments 1 to 106, wherein the amino acid sequence of the Type V Cas protein comprises an amino acid sequence that is at least 85% identical to the amino acid sequence of the BH domain of the reference protein sequence.

115. The Type V Cas protein of any one of embodiments 1 to 106, wherein the amino acid sequence of the Type V Cas protein comprises an amino acid sequence that is at least 90% identical to the amino acid sequence of the BH domain of the reference protein sequence.

116. The Type V Cas protein of any one of embodiments 1 to 106, wherein the amino acid sequence of the Type V Cas protein comprises an amino acid sequence that is at least 95% identical to the amino acid sequence of the BH domain of the reference protein sequence.

117. The Type V Cas protein of any one of embodiments 1 to 106, wherein the amino acid sequence of the Type V Cas protein comprises an amino acid sequence that is at least 96% identical to the amino acid sequence of the BH domain of the reference protein sequence.

118. The Type V Cas protein of any one of embodiments 1 to 106, wherein the amino acid sequence of the Type V Cas protein comprises an amino acid sequence that is at least 97% identical to the amino acid sequence of the BH domain of the reference protein sequence.

119. The Type V Cas protein of any one of embodiments 1 to 106, wherein the amino acid sequence of the Type V Cas protein comprises an amino acid sequence that is at least 98% identical to the amino acid sequence of the BH domain of the reference protein sequence.

120. The Type V Cas protein of any one of embodiments 1 to 106, wherein the amino acid sequence of the Type V Cas protein comprises an amino acid sequence that is at least 99% identical to the amino acid sequence of the BH domain of the reference protein sequence.

121. The Type V Cas protein of any one of embodiments 1 to 106, wherein the amino acid sequence of the Type V Cas protein comprises an amino acid sequence that is identical to the amino acid sequence of the BH domain of the reference protein sequence.

122. The Type V Cas protein of any one of embodiments 1 to 121, wherein the amino acid sequence of the Type V Cas protein comprises an amino acid sequence that is at least 50% identical to the amino acid sequence of the RuvC-II domain of the reference protein sequence.

123. The Type V Cas protein of any one of embodiments 1 to 121, wherein the amino acid sequence of the Type V Cas protein comprises an amino acid sequence that is at least 55% identical to the amino acid sequence of the RuvC-II domain of the reference protein sequence.

124. The Type V Cas protein of any one of embodiments 1 to 121, wherein the amino acid sequence of the Type V Cas protein comprises an amino acid sequence that is at least 60% identical to the amino acid sequence of the RuvC-II domain of the reference protein sequence.

125. The Type V Cas protein of any one of embodiments 1 to 121, wherein the amino acid sequence of the Type V Cas protein comprises an amino acid sequence that is at least 65% identical to the amino acid sequence of the RuvC-II domain of the reference protein sequence.

126. The Type V Cas protein of any one of embodiments 1 to 121, wherein the amino acid sequence of the Type V Cas protein comprises an amino acid sequence that is at least 70% identical to the amino acid sequence of the RuvC-II domain of the reference protein sequence.

127. The Type V Cas protein of any one of embodiments 1 to 121, wherein the amino acid sequence of the Type V Cas protein comprises an amino acid sequence that is at least 75% identical to the amino acid sequence of the RuvC-II domain of the reference protein sequence.

128. The Type V Cas protein of any one of embodiments 1 to 121, wherein the amino acid sequence of the Type V Cas protein comprises an amino acid sequence that is at least 80% identical to the amino acid sequence of the RuvC-II domain of the reference protein sequence.

129. The Type V Cas protein of any one of embodiments 1 to 121, wherein the amino acid sequence of the Type V Cas protein comprises an amino acid sequence that is at least 85% identical to the amino acid sequence of the RuvC-II domain of the reference protein sequence.

130. The Type V Cas protein of any one of embodiments 1 to 121, wherein the amino acid sequence of the Type V Cas protein comprises an amino acid sequence that is at least 90% identical to the amino acid sequence of the RuvC-II domain of the reference protein sequence.

131. The Type V Cas protein of any one of embodiments 1 to 121, wherein the amino acid sequence of the Type V Cas protein comprises an amino acid sequence that is at least 95% identical to the amino acid sequence of the RuvC-II domain of the reference protein sequence.

132. The Type V Cas protein of any one of embodiments 1 to 121, wherein the amino acid sequence of the Type V Cas protein comprises an amino acid sequence that is at least 96% identical to the amino acid sequence of the RuvC-II domain of the reference protein sequence.

133. The Type V Cas protein of any one of embodiments 1 to 121, wherein the amino acid sequence of the Type V Cas protein comprises an amino acid sequence that is at least 97% identical to the amino acid sequence of the RuvC-II domain of the reference protein sequence.

134. The Type V Cas protein of any one of embodiments 1 to 121, wherein the amino acid sequence of the Type V Cas protein comprises an amino acid sequence that is at least 98% identical to the amino acid sequence of the RuvC-II domain of the reference protein sequence.

135. The Type V Cas protein of any one of embodiments 1 to 121, wherein the amino acid sequence of the Type V Cas protein comprises an amino acid sequence that is at least 99% identical to the amino acid sequence of the RuvC-II domain of the reference protein sequence.

136. The Type V Cas protein of any one of embodiments 1 to 121, wherein the amino acid sequence of the Type V Cas protein comprises an amino acid sequence that is identical to the amino acid sequence of the RuvC-II domain of the reference protein sequence.

137. The Type V Cas protein of any one of embodiments 1 to 136, wherein the amino acid sequence of the Type V Cas protein comprises an amino acid sequence that is at least 50% identical to the amino acid sequence of the NUC domain of the reference protein sequence.

138. The Type V Cas protein of any one of embodiments 1 to 136, wherein the amino acid sequence of the Type V Cas protein comprises an amino acid sequence that is at least 55% identical to the amino acid sequence of the NUC domain of the reference protein sequence.

139. The Type V Cas protein of any one of embodiments 1 to 136, wherein the amino acid sequence of the Type V Cas protein comprises an amino acid sequence that is at least 60% identical to the amino acid sequence of the NUC domain of the reference protein sequence.

140. The Type V Cas protein of any one of embodiments 1 to 136, wherein the amino acid sequence of the Type V Cas protein comprises an amino acid sequence that is at least 65% identical to the amino acid sequence of the NUC domain of the reference protein sequence.

141. The Type V Cas protein of any one of embodiments 1 to 136, wherein the amino acid sequence of the Type V Cas protein comprises an amino acid sequence that is at least 70% identical to the amino acid sequence of the NUC domain of the reference protein sequence.

142. The Type V Cas protein of any one of embodiments 1 to 136, wherein the amino acid sequence of the Type V Cas protein comprises an amino acid sequence that is at least 75% identical to the amino acid sequence of the NUC domain of the reference protein sequence.

143. The Type V Cas protein of any one of embodiments 1 to 136, wherein the amino acid sequence of the Type V Cas protein comprises an amino acid sequence that is at least 80% identical to the amino acid sequence of the NUC domain of the reference protein sequence.

144. The Type V Cas protein of any one of embodiments 1 to 136, wherein the amino acid sequence of the Type V Cas protein comprises an amino acid sequence that is at least 85% identical to the amino acid sequence of the NUC domain of the reference protein sequence.

145. The Type V Cas protein of any one of embodiments 1 to 136, wherein the amino acid sequence of the Type V Cas protein comprises an amino acid sequence that is at least 90% identical to the amino acid sequence of the NUC domain of the reference protein sequence.

146. The Type V Cas protein of any one of embodiments 1 to 136, wherein the amino acid sequence of the Type V Cas protein comprises an amino acid sequence that is at least 95% identical to the amino acid sequence of the NUC domain of the reference protein sequence.

147. The Type V Cas protein of any one of embodiments 1 to 136, wherein the amino acid sequence of the Type V Cas protein comprises an amino acid sequence that is at least 96% identical to the amino acid sequence of the NUC domain of the reference protein sequence.

148. The Type V Cas protein of any one of embodiments 1 to 136, wherein the amino acid sequence of the Type V Cas protein comprises an amino acid sequence that is at least 97% identical to the amino acid sequence of the NUC domain of the reference protein sequence.

149. The Type V Cas protein of any one of embodiments 1 to 136, wherein the amino acid sequence of the Type V Cas protein comprises an amino acid sequence that is at least 98% identical to the amino acid sequence of the NUC domain of the reference protein sequence.

150. The Type V Cas protein of any one of embodiments 1 to 136, wherein the amino acid sequence of the Type V Cas protein comprises an amino acid sequence that is at least 99% identical to the amino acid sequence of the NUC domain of the reference protein sequence.

151. The Type V Cas protein of any one of embodiments 1 to 136, wherein the amino acid sequence of the Type V Cas protein comprises an amino acid sequence that is identical to the amino acid sequence of the NUC domain of the reference protein sequence.

152. The Type V Cas protein of any one of embodiments 1 to 151, wherein the amino acid sequence of the Type V Cas protein comprises an amino acid sequence that is at least 50% identical to the amino acid sequence of the RuvC-III domain of the reference protein sequence.

153. The Type V Cas protein of any one of embodiments 1 to 151, wherein the amino acid sequence of the Type V Cas protein comprises an amino acid sequence that is at least 55% identical to the amino acid sequence of the RuvC-III domain of the reference protein sequence.

154. The Type V Cas protein of any one of embodiments 1 to 151, wherein the amino acid sequence of the Type V Cas protein comprises an amino acid sequence that is at least 60% identical to the amino acid sequence of the RuvC-III domain of the reference protein sequence.

155. The Type V Cas protein of any one of embodiments 1 to 151, wherein the amino acid sequence of the Type V Cas protein comprises an amino acid sequence that is at least 65% identical to the amino acid sequence of the RuvC-III domain of the reference protein sequence.

156. The Type V Cas protein of any one of embodiments 1 to 151, wherein the amino acid sequence of the Type V Cas protein comprises an amino acid sequence that is at least 70% identical to the amino acid sequence of the RuvC-III domain of the reference protein sequence.

157. The Type V Cas protein of any one of embodiments 1 to 151, wherein the amino acid sequence of the Type V Cas protein comprises an amino acid sequence that is at least 75% identical to the amino acid sequence of the RuvC-III domain of the reference protein sequence.

158. The Type V Cas protein of any one of embodiments 1 to 151, wherein the amino acid sequence of the Type V Cas protein comprises an amino acid sequence that is at least 80% identical to the amino acid sequence of the RuvC-III domain of the reference protein sequence.

159. The Type V Cas protein of any one of embodiments 1 to 151, wherein the amino acid sequence of the Type V Cas protein comprises an amino acid sequence that is at least 85% identical to the amino acid sequence of the RuvC-III domain of the reference protein sequence.

160. The Type V Cas protein of any one of embodiments 1 to 151, wherein the amino acid sequence of the Type V Cas protein comprises an amino acid sequence that is at least 90% identical to the amino acid sequence of the RuvC-III domain of the reference protein sequence.

161. The Type V Cas protein of any one of embodiments 1 to 151, wherein the amino acid sequence of the Type V Cas protein comprises an amino acid sequence that is at least 95% identical to the amino acid sequence of the RuvC-III domain of the reference protein sequence.

162. The Type V Cas protein of any one of embodiments 1 to 151, wherein the amino acid sequence of the Type V Cas protein comprises an amino acid sequence that is at least 96% identical to the amino acid sequence of the RuvC-III domain of the reference protein sequence.

163. The Type V Cas protein of any one of embodiments 1 to 151, wherein the amino acid sequence of the Type V Cas protein comprises an amino acid sequence that is at least 97% identical to the amino acid sequence of the RuvC-III domain of the reference protein sequence.

164. The Type V Cas protein of any one of embodiments 1 to 151, wherein the amino acid sequence of the Type V Cas protein comprises an amino acid sequence that is at least 98% identical to the amino acid sequence of the RuvC-III domain of the reference protein sequence.

165. The Type V Cas protein of any one of embodiments 1 to 151, wherein the amino acid sequence of the Type V Cas protein comprises an amino acid sequence that is at least 99% identical to the amino acid sequence of the RuvC-III domain of the reference protein sequence.

166. The Type V Cas protein of any one of embodiments 1 to 151, wherein the amino acid sequence of the Type V Cas protein comprises an amino acid sequence that is identical to the amino acid sequence of the RuvC-III domain of the reference protein sequence.

167. The Type V Cas protein of embodiment 1, wherein the amino acid sequence of the Type V Cas protein comprises an amino acid sequence that is at least 55% identical to the full length of the reference protein sequence.

168. The Type V Cas protein of embodiment 1, wherein the amino acid sequence of the Type V Cas protein comprises an amino acid sequence that is at least 60% identical to the full length of the reference protein sequence.

169. The Type V Cas protein of embodiment 1, wherein the amino acid sequence of the Type V Cas protein comprises an amino acid sequence that is at least 65% identical to the full length of the reference protein sequence.

170. The Type V Cas protein of embodiment 1, wherein the amino acid sequence of the Type V Cas protein comprises an amino acid sequence that is at least 70% identical to the full length of the reference protein sequence.

171. The Type V Cas protein of embodiment 1, wherein the amino acid sequence of the Type V Cas protein comprises an amino acid sequence that is at least 75% identical to the full length of the reference protein sequence.

172. The Type V Cas protein of embodiment 1, wherein the amino acid sequence of the Type V Cas protein comprises an amino acid sequence that is at least 80% identical to the full length of the reference protein sequence.

173. The Type V Cas protein of embodiment 1, wherein the amino acid sequence of the Type V Cas protein comprises an amino acid sequence that is at least 85% identical to the full length of the reference protein sequence.

174. The Type V Cas protein of embodiment 1, wherein the amino acid sequence of the Type V Cas protein comprises an amino acid sequence that is at least 90% identical to the full length of the reference protein sequence.

175. The Type V Cas protein of embodiment 1, wherein the amino acid sequence of the Type V Cas protein comprises an amino acid sequence that is at least 95% identical to the full length of the reference protein sequence.

176. The Type V Cas protein of embodiment 1, wherein the amino acid sequence of the Type V Cas protein comprises an amino acid sequence that is at least 96% identical to the full length of the reference protein sequence.

177. The Type V Cas protein of embodiment 1, wherein the amino acid sequence of the Type V Cas protein comprises an amino acid sequence that is at least 97% identical to the full length of the reference protein sequence.

178. The Type V Cas protein of embodiment 1, wherein the amino acid sequence of the Type V Cas protein comprises an amino acid sequence that is at least 98% identical to the full length of the reference protein sequence.

179. The Type V Cas protein of embodiment 1, wherein the amino acid sequence of the Type V Cas protein comprises an amino acid sequence that is at least 99% identical to the full length of the reference protein sequence.

180. The Type V Cas protein of embodiment 1, wherein the amino acid sequence of the Type V Cas protein comprises an amino acid sequence that is identical to the full length of the reference protein sequence.

181. The Type V Cas protein of any one of embodiments 1 to 180, which is a chimeric Type V Cas protein.

182. The Type V Cas protein of any one of embodiments 1 to 181, which is a fusion protein.

183. The Type V Cas protein of embodiment 182, which comprises one or more nuclear localization signals.

184. The Type V Cas protein of embodiment 183, which comprises two or more nuclear localization signals.

185. The Type V Cas protein of embodiment 183 or embodiment 184, which comprises an N-terminal nuclear localization signal.

186. The Type V Cas protein of any one of embodiments 183 to 185, which comprises a C-terminal nuclear localization signal.

187. The Type V Cas protein of any one of embodiments 183 to 186, which comprises an N-terminal nuclear localization signal and a C-terminal nuclear localization signal.

188. The Type V Cas protein of any one of embodiments 183 to 187, wherein the amino acid sequence of one or more of the nuclear localization signals comprises the amino acid sequence

(SEQ ID NO: 122)
KRTADGSEFESPKKKRKV,
(SEQ ID NO: 123)
PKKKRKV,
(SEQ ID NO: 124)
PKKKRRV,
(SEQ ID NO: 125)
KRPAATKKAGQAKKKK,
(SEQ ID NO: 126)
YGRKKRRQRRR,
(SEQ ID NO: 127)
RKKRRQRRR,
(SEQ ID NO: 128)
PAAKRVKLD,
(SEQ ID NO: 129)
RQRRNELKRSP,
(SEQ ID NO: 130)
VSRKRPRP,
(SEQ ID NO: 131)
PPKKARED,
(SEQ ID NO: 132)
PQPKKKPL,
(SEQ ID NO: 133)
SALIKKKKKMAP,
(SEQ ID NO: 134)
PKQKKRK,
(SEQ ID NO: 135)
RKLKKKIKKL,
(SEQ ID NO: 136)
REKKKFLKRR,
(SEQ ID NO: 137)
KRKGDEVDGVDEVAKKKSKK,
(SEQ ID NO: 138)
RKCLQAGMNLEARKTKK,
(SEQ ID NO: 139)
NQSSNFGPMKGGNFGGRSSGPYGGGGQYFAKPRNQGGY,
(SEQ ID NO: 140)
RMRIZFKNKGKDTAELRRRRVEVSVELRKAKKDEQILKRRNV,
or
(SEQ ID NO: 178)
SSDDEATADSQHAAPPKKKRKV.

189. The Type V Cas protein of embodiment 188, wherein the amino acid sequence of one or more of the nuclear localization signals comprises the amino acid sequence KRTADGSEFESPKKKRKV (SEQ ID NO: 122).

190. The Type V Cas protein of embodiment 188, wherein the amino acid sequence of one or more of the nuclear localization signals comprises the amino acid sequence PKKKRKV (SEQ ID NO: 123).

191. The Type V Cas protein of embodiment 188, wherein the amino acid sequence of one or more of the nuclear localization signals comprises the amino acid sequence PKKKRRV (SEQ ID NO: 124).

192. The Type V Cas protein of embodiment 188, wherein the amino acid sequence of one or more of the nuclear localization signals comprises the amino acid sequence KRPAATKKAGQAKKKK (SEQ ID NO: 125).

193. The Type V Cas protein of embodiment 188, wherein the amino acid sequence of one or more of the nuclear localization signals comprises the amino acid sequence YGRKKRRQRRR (SEQ ID NO: 126).

194. The Type V Cas protein of embodiment 188, wherein the amino acid sequence of one or more of the nuclear localization signals comprises the amino acid sequence RKKRRQRRR (SEQ ID NO: 127).

195. The Type V Cas protein of embodiment 188, wherein the amino acid sequence of one or more of the nuclear localization signals comprises the amino acid sequence PAAKRVKLD (SEQ ID NO: 128).

196. The Type V Cas protein of embodiment 188, wherein the amino acid sequence of one or more of the nuclear localization signals comprises the amino acid sequence RQRRNELKRSP (SEQ ID NO: 129).

197. The Type V Cas protein of embodiment 188, wherein the amino acid sequence of one or more of the nuclear localization signals comprises the amino acid sequence VSRKRPRP (SEQ ID NO: 130).

198. The Type V Cas protein of embodiment 188, wherein the amino acid sequence of one or more of the nuclear localization signals comprises the amino acid sequence PPKKARED (SEQ ID NO: 131).

199. The Type V Cas protein of embodiment 188, wherein the amino acid sequence of one or more of the nuclear localization signals comprises the amino acid sequence PQPKKKPL (SEQ ID NO: 132).

200. The Type V Cas protein of embodiment 188, wherein the amino acid sequence of one or more of the nuclear localization signals comprises the amino acid sequence SALIKKKKKMAP (SEQ ID NO: 133).

201. The Type V Cas protein of embodiment 188, wherein the amino acid sequence of one or more of the nuclear localization signals comprises the amino acid sequence PKQKKRK (SEQ ID NO: 134).

202. The Type V Cas protein of embodiment 188, wherein the amino acid sequence of one or more of the nuclear localization signals comprises the amino acid sequence RKLKKKIKKL (SEQ ID NO: 135).

203. The Type V Cas protein of embodiment 188, wherein the amino acid sequence of one or more of the nuclear localization signals comprises the amino acid sequence REKKKFLKRR (SEQ ID NO: 136).

204. The Type V Cas protein of embodiment 188, wherein the amino acid sequence of one or more of the nuclear localization signals comprises the amino acid sequence

(SEQ ID NO: 137)
KRKGDEVDGVDEVAKKKSKK.

205. The Type V Cas protein of embodiment 188, wherein the amino acid sequence of one or more of the nuclear localization signals comprises the amino acid sequence RKCLQAGMNLEARKTKK (SEQ ID NO:138).

206. The Type V Cas protein of embodiment 188, wherein the amino acid sequence of one or more of the nuclear localization signals comprises the amino acid sequence

(SEQ ID NO: 139)
NQSSNFGPMKGGNFGGRSSGPYGGGGQYFAKPRNQGGY.

207. The Type V Cas protein of embodiment 188, wherein the amino acid sequence of one or more of the nuclear localization signals comprises the amino acid sequence

(SEQ ID NO: 140)
RMRIZFKNKGKDTAELRRRRVEVSVELRKAKKDEQILKRRNV.

208. The Type V Cas protein of embodiment 188, wherein the amino acid sequence of one or more of the nuclear localization signals comprises the amino acid sequence

(SEQ ID NO: 178)
SSDDEATADSQHAAPPKKKRKV.

209. The Type V Cas protein of any one of embodiments 183 to 187, wherein the amino acid sequence of one or more of the nuclear localization signals comprises the amino acid sequence

(SEQ ID NO: 179)
PKKKRKVG.

210. The Type V Cas protein of any one of embodiments 183 to 187, wherein the amino acid sequence of one or more of the nuclear localization signals comprises the amino acid sequence

(SEQ ID NO: 180)
GRSSDDEATADSQHAAPPKKKRKV.

211. The Type V Cas protein of any one of embodiments 183 to 210, wherein the amino acid sequence of each nuclear localization signal is the same.

212. The Type V Cas protein of any one of embodiments 181 to 211, which comprises a fusion partner which is a DNA, RNA or protein modification enzyme, optionally wherein the DNA, RNA or protein modification enzyme is an adenosine deaminase, a cytidine deaminase, a reverse transcriptase, a guanosyl transferase, a DNA methyltransferase, a RNA methyltransferase, a DNA demethylase, a RNA demethylase, a dioxygenase, a polyadenylate polymerase, a pseudouridine synthase, an acetyltransferase, a deacetylase, a ubiquitin-ligase, a deubiquitinase, a kinase, a phosphatase, a NEDD8-ligase, a de-NEDDylase, a SUMO-ligase, a deSUMOylase, a histone deacetylase, a histone acetyltransferase, a histone methyltransferase, or a histone demethylase.

213. The Type V Cas protein of any one of embodiments 181 to 212, which comprises a means for deaminating a nucleobase, optionally wherein the means for deaminating a nucleobase is a deaminase, e.g., an adenosine deaminase or cytidine deaminase.

214. The Type V Cas protein of any one of embodiments 181 to 213, which comprises a fusion partner comprising a deaminase, optionally wherein the deaminase is an adenosine deaminase or cytidine deaminase.

215. The Type V Cas protein of embodiment 214, wherein the amino acid sequence of the deaminase comprises an amino acid sequence having at least 50%, at least 60%, at least 70%, at least 80%, at least 90%, at least 95%, at least 97%, at least 98%, at least 99%, or 100% sequence identity to any one of SEQ ID NOS: 214-249.

216. The Type V Cas protein of any one of embodiments 181 to 212, which comprises a means for deaminating adenosine, optionally wherein the means for deaminating adenosine is an adenosine deaminase.

217. The Type V Cas protein of any one of embodiments 181 to 212, which comprises a fusion partner which is an adenosine deaminase, optionally wherein the amino acid sequence of the adenosine deaminase comprises an amino acid sequence having at least 50%, at least 60%, at least 70%, at least 80%, at least 90%, at least 95%, at least 97%, at least 98%, at least 99%, or 100% sequence identity with SEQ ID NO: 166, optionally wherein the adenosine deaminase is the adenosine deaminase moiety contained in the adenine base editor ABE8e.

218. The Type V Cas protein of any one of embodiments 181 to 212, which comprises a means for deaminating cytidine, optionally wherein the means for deaminating cytidine is a cytidine deaminase.

219. The Type V Cas protein of any one of embodiments 181 to 212, which comprises a fusion partner which is a cytidine deaminase.

220. The Type V Cas protein of any one of embodiments 181 to 219, which comprises a fusion partner comprising a UGI domain, optionally wherein the amino acid sequence of the UGI domain comprises an amino acid sequence having at least 50%, at least 60%, at least 70%, at least 80%, at least 90%, at least 95%, at least 97%, at least 98%, at least 99%, or 100% sequence identity to SEQ ID NO: 250.

221. The Type V Cas protein of any one of embodiments 181 to 220, which comprises a means for repressing gene expression, optionally wherein the means for repressing gene expression comprises a transcriptional repressor or effector domain thereof.

222. The Type V Cas protein of any one of embodiments 181 to 220, which comprises a fusion partner comprising a transcriptional repressor or effector domain thereof.

223. The Type V Cas protein of embodiment 221 or embodiment 222, wherein the amino acid sequence of the transcriptional repressor or effector domain thereof comprises an amino acid sequence having at least 50%, at least 60%, at least 70%, at least 80%, at least 90%, at least 95%, at least 97%, at least 98%, at least 99%, or 100% sequence identity to any one of SEQ ID NOS: 251-255.

224. The Type V Cas protein of any one of embodiments 181 to 212, which comprises a means for synthesizing DNA from a single-stranded template, optionally wherein the means for synthesizing DNA from a single-stranded template is a reverse transcriptase.

225. The Type V Cas protein of any one of embodiments 181 to 212, which comprises a fusion partner which is a reverse transcriptase.

226. The Type V Cas protein of embodiment 224 or embodiment 225, wherein the amino acid sequence of the reverse transcriptase comprises an amino acid sequence having at least 50%, at least 60%, at least 70%, at least 80%, at least 90%, at least 95%, at least 97%, at least 98%, at least 99%, or 100% sequence identity to SEQ ID NO:256 or SEQ ID NO:257.

227. The Type V Cas protein of any one of embodiments 181 to 225, which comprises a tag. 228. The Type V Cas protein of embodiment 226, wherein the tag is a SV5 tag, optionally wherein the SV5 tag comprises the amino acid sequence GKPIPNPLLGLDST (SEQ ID NO:141) or IPNPLLGLD (SEQ ID NO:142).

229. The Type V Cas protein of any one of embodiments 1 to 228, wherein the reference protein sequence is SEQ ID NO:1.

230. The Type V Cas protein of embodiment 229, whose amino acid sequence comprises the amino acid sequence of SEQ ID NO:1.

231. The Type V Cas protein of any one of embodiments 1 to 228, wherein the reference protein sequence is SEQ ID NO:2.

232. The Type V Cas protein of any one of embodiments 229 to 231, whose amino acid sequence comprises the amino acid sequence of SEQ ID NO:2.

233. The Type V Cas protein of embodiment 229 or embodiment 230, whose amino acid sequence comprises the amino acid sequence of SEQ ID NO:3.

234. The Type V Cas protein of any one of embodiments 1 to 228, wherein the reference protein sequence is SEQ ID NO:7.

235. The Type V Cas protein of embodiment 234, whose amino acid sequence comprises the amino acid sequence of SEQ ID NO:7.

236. The Type V Cas protein of any one of embodiments 1 to 228, wherein the reference protein sequence is SEQ ID NO:8.

237. The Type V Cas protein of any one of embodiments 234 to 236, whose amino acid sequence comprises the amino acid sequence of SEQ ID NO:8.

238. The Type V Cas protein of embodiment 234 or embodiment 235, whose amino acid sequence comprises the amino acid sequence of SEQ ID NO:9.

239. The Type V Cas protein of any one of embodiments 1 to 228, wherein the reference protein sequence is SEQ ID NO:13.

240. The Type V Cas protein of embodiment 239, whose amino acid sequence comprises the amino acid sequence of SEQ ID NO: 13.

241. The Type V Cas protein of any one of embodiments 1 to 228, wherein the reference protein sequence is SEQ ID NO:14.

242. The Type V Cas protein of any one of embodiments 239 to 241, whose amino acid sequence comprises the amino acid sequence of SEQ ID NO: 14.

243. The Type V Cas protein of embodiment 239 or embodiment 240, whose amino acid sequence comprises the amino acid sequence of SEQ ID NO: 15.

244. The Type V Cas protein of any one of embodiments 1 to 228, wherein the reference protein sequence is SEQ ID NO:19.

245. The Type V Cas protein of embodiment 244, whose amino acid sequence comprises the amino acid sequence of SEQ ID NO:19.

246. The Type V Cas protein of any one of embodiments 1 to 228, wherein the reference protein sequence is SEQ ID NO:20.

247. The Type V Cas protein of any one of embodiments 244 to 246, whose amino acid sequence comprises the amino acid sequence of SEQ ID NO:20.

248. The Type V Cas protein of embodiment 244 or embodiment 245, whose amino acid sequence comprises the amino acid sequence of SEQ ID NO:21.

249. The Type V Cas protein of any one of embodiments 1 to 228, wherein the reference protein sequence is SEQ ID NO:25.

250. The Type V Cas protein of embodiment 249, whose amino acid sequence comprises the amino acid sequence of SEQ ID NO:25.

251. The Type V Cas protein of any one of embodiments 1 to 228, wherein the reference protein sequence is SEQ ID NO:26.

252. The Type V Cas protein of any one of embodiments 249 to 251, whose amino acid sequence comprises the amino acid sequence of SEQ ID NO:26.

253. The Type V Cas protein of embodiment 250 or embodiment 251, whose amino acid sequence comprises the amino acid sequence of SEQ ID NO:27.

254. The Type V Cas protein of any one of embodiments 1 to 228, wherein the reference protein sequence is SEQ ID NO:31.

255. The Type V Cas protein of embodiment 254, whose amino acid sequence comprises the amino acid sequence of SEQ ID NO:31.

256. The Type V Cas protein of any one of embodiments 1 to 228, wherein the reference protein sequence is SEQ ID NO:32.

257. The Type V Cas protein of any one of embodiments 255 to 256, whose amino acid sequence comprises the amino acid sequence of SEQ ID NO:32.

258. The Type V Cas protein of embodiment 254 or embodiment 255, whose amino acid sequence comprises the amino acid sequence of SEQ ID NO:33.

259. The Type V Cas protein of any one of embodiments 1 to 228, wherein the reference protein sequence is SEQ ID NO:37.

260. The Type V Cas protein of embodiment 259, whose amino acid sequence comprises the amino acid sequence of SEQ ID NO:37.

261. The Type V Cas protein of any one of embodiments 1 to 228, wherein the reference protein sequence is SEQ ID NO:38.

262. The Type V Cas protein of any one of embodiments 259 to 261, whose amino acid sequence comprises the amino acid sequence of SEQ ID NO:38.

263. The Type V Cas protein of embodiment 259 or embodiment 260, whose amino acid sequence comprises the amino acid sequence of SEQ ID NO:39.

264. The Type V Cas protein of any one of embodiments 1 to 228, wherein the reference protein sequence is SEQ ID NO:43.

265. The Type V Cas protein of embodiment 264, whose amino acid sequence comprises the amino acid sequence of SEQ ID NO:43.

266. The Type V Cas protein of any one of embodiments 1 to 228, wherein the reference protein sequence is SEQ ID NO:44.

267. The Type V Cas protein of any one of embodiments 264 to 266, whose amino acid sequence comprises the amino acid sequence of SEQ ID NO:44.

268. The Type V Cas protein of embodiment 264 or embodiment 265, whose amino acid sequence comprises the amino acid sequence of SEQ ID NO:45.

269. The Type V Cas protein of any one of embodiments 1 to 228, wherein the reference protein sequence is SEQ ID NO:49.

270. The Type V Cas protein of embodiment 269, whose amino acid sequence comprises the amino acid sequence of SEQ ID NO:49.

271. The Type V Cas protein of any one of embodiments 1 to 228, wherein the reference protein sequence is SEQ ID NO:50.

272. The Type V Cas protein of any one of embodiments 269 to 271, whose amino acid sequence comprises the amino acid sequence of SEQ ID NO:50.

273. The Type V Cas protein of embodiment 269 or embodiment 270, whose amino acid sequence comprises the amino acid sequence of SEQ ID NO:51.

274. The Type V Cas protein of any one of embodiments 1 to 228, wherein the reference protein sequence is SEQ ID NO:55.

275. The Type V Cas protein of embodiment 274, whose amino acid sequence comprises the amino acid sequence of SEQ ID NO:55.

276. The Type V Cas protein of any one of embodiments 1 to 228, wherein the reference protein sequence is SEQ ID NO:56.

277. The Type V Cas protein of any one of embodiments 274 to 276, whose amino acid sequence comprises the amino acid sequence of SEQ ID NO:56.

278. The Type V Cas protein of embodiment 274 or embodiment 275, whose amino acid sequence comprises the amino acid sequence of SEQ ID NO:57.

279. The Type V Cas protein of any one of embodiments 1 to 228, wherein the reference protein sequence is SEQ ID NO:61.

280. The Type V Cas protein of embodiment 279, whose amino acid sequence comprises the amino acid sequence of SEQ ID NO:61.

281. The Type V Cas protein of any one of embodiments 1 to 228, wherein the reference protein sequence is SEQ ID NO:62.

282. The Type V Cas protein of any one of embodiments 279 to 281, whose amino acid sequence comprises the amino acid sequence of SEQ ID NO:62.

283. The Type V Cas protein of embodiment 279 or embodiment 280, whose amino acid sequence comprises the amino acid sequence of SEQ ID NO:63.

284. The Type V Cas protein of any one of embodiments 1 to 228, wherein the reference protein sequence is SEQ ID NO:67.

285. The Type V Cas protein of embodiment 284, whose amino acid sequence comprises the amino acid sequence of SEQ ID NO:67.

286. The Type V Cas protein of any one of embodiments 1 to 228, wherein the reference protein sequence is SEQ ID NO:68.

287. The Type V Cas protein of any one of embodiments 284 to 286, whose amino acid sequence comprises the amino acid sequence of SEQ ID NO:68.

288. The Type V Cas protein of embodiment 284 or embodiment 285, whose amino acid sequence comprises the amino acid sequence of SEQ ID NO:69.

289. The Type V Cas protein of any one of embodiments 1 to 228, wherein the reference protein sequence is SEQ ID NO:73.

290. The Type V Cas protein of embodiment 289, whose amino acid sequence comprises the amino acid sequence of SEQ ID NO:73.

291. The Type V Cas protein of any one of embodiments 1 to 228, wherein the reference protein sequence is SEQ ID NO:74.

292. The Type V Cas protein of any one of embodiments 289 to 291, whose amino acid sequence comprises the amino acid sequence of SEQ ID NO:74.

293. The Type V Cas protein of embodiment 289 or embodiment 290, whose amino acid sequence comprises the amino acid sequence of SEQ ID NO:75.

294. The Type V Cas protein of any one of embodiments 1 to 228, wherein the reference protein sequence is SEQ ID NO:79.

295. The Type V Cas protein of embodiment 294, whose amino acid sequence comprises the amino acid sequence of SEQ ID NO:79.

296. The Type V Cas protein of any one of embodiments 1 to 228, wherein the reference protein sequence is SEQ ID NO:80.

297. The Type V Cas protein of any one of embodiments 294 to 296, whose amino acid sequence comprises the amino acid sequence of SEQ ID NO:80.

298. The Type V Cas protein of embodiment 294 or embodiment 295, whose amino acid sequence comprises the amino acid sequence of SEQ ID NO:81.

299. The Type V Cas protein of any one of embodiments 1 to 228, wherein the reference protein sequence is SEQ ID NO:85.

300. The Type V Cas protein of embodiment 299, whose amino acid sequence comprises the amino acid sequence of SEQ ID NO:85.

301. The Type V Cas protein of any one of embodiments 1 to 228, wherein the reference protein sequence is SEQ ID NO:86.

302. The Type V Cas protein of any one of embodiments 299 to 301, whose amino acid sequence comprises the amino acid sequence of SEQ ID NO:86.

303. The Type V Cas protein of embodiment 299 or embodiment 300, whose amino acid sequence comprises the amino acid sequence of SEQ ID NO:87.

304. The Type V Cas protein of any one of embodiments 1 to 228, wherein the reference protein sequence is SEQ ID NO:91.

305. The Type V Cas protein of embodiment 304, whose amino acid sequence comprises the amino acid sequence of SEQ ID NO:91.

306. The Type V Cas protein of any one of embodiments 1 to 228, wherein the reference protein sequence is SEQ ID NO:92.

307. The Type V Cas protein of any one of embodiments 304 to 306, whose amino acid sequence comprises the amino acid sequence of SEQ ID NO:92.

308. The Type V Cas protein of embodiment 304 or embodiment 305, whose amino acid sequence comprises the amino acid sequence of SEQ ID NO:93.

309. The Type V Cas protein of any one of embodiments 1 to 228, wherein the reference protein sequence is SEQ ID NO:97.

310. The Type V Cas protein of embodiment 309, whose amino acid sequence comprises the amino acid sequence of SEQ ID NO:97.

311. The Type V Cas protein of any one of embodiments 1 to 228, wherein the reference protein sequence is SEQ ID NO:98.

312. The Type V Cas protein of any one of embodiments 309 to 311, whose amino acid sequence comprises the amino acid sequence of SEQ ID NO:98.

313. The Type V Cas protein of embodiment 309 or embodiment 310, whose amino acid sequence comprises the amino acid sequence of SEQ ID NO:99.

314. The Type V Cas protein of any one of embodiments 1 to 228, wherein the reference protein sequence is SEQ ID NO:103.

315. The Type V Cas protein of embodiment 314, whose amino acid sequence comprises the amino acid sequence of SEQ ID NO: 103.

316. The Type V Cas protein of any one of embodiments 1 to 228, wherein the reference protein sequence is SEQ ID NO:104.

317. The Type V Cas protein of any one of embodiments 314 to 316, whose amino acid sequence comprises the amino acid sequence of SEQ ID NO: 104.

318. The Type V Cas protein of embodiment 314 or embodiment 315, whose amino acid sequence comprises the amino acid sequence of SEQ ID NO: 105.

319. The Type V Cas protein of any one of embodiments 1 to 228, wherein the reference protein sequence is SEQ ID NO:109.

320. The Type V Cas protein of embodiment 319, whose amino acid sequence comprises the amino acid sequence of SEQ ID NO: 109.

321. The Type V Cas protein of any one of embodiments 1 to 228, wherein the reference protein sequence is SEQ ID NO:110.

322. The Type V Cas protein of any one of embodiments 319 to 321, whose amino acid sequence comprises the amino acid sequence of SEQ ID NO: 110.

323. The Type V Cas protein of embodiment 319 or embodiment 320, whose amino acid sequence comprises the amino acid sequence of SEQ ID NO:111.

324. The Type V Cas protein of any one of embodiments 1 to 228, wherein the reference protein sequence is SEQ ID NO:115.

325. The Type V Cas protein of embodiment 324, whose amino acid sequence comprises the amino acid sequence of SEQ ID NO: 115.

326. The Type V Cas protein of any one of embodiments 1 to 228, wherein the reference protein sequence is SEQ ID NO:116.

327. The Type V Cas protein of any one of embodiments 324 to 326, whose amino acid sequence comprises the amino acid sequence of SEQ ID NO: 116.

328. The Type V Cas protein of embodiment 324 or embodiment 325, whose amino acid sequence comprises the amino acid sequence of SEQ ID NO: 117.

329. A Type V Cas protein whose amino acid sequence is identical to a Type V Cas protein of any one of embodiments 1 to 328 except for one or more amino acid substitutions relative to the reference sequence that provide nickase activity, optionally wherein the one or more amino acid substitutions comprise a substitution (e.g., alanine substitution) at a position corresponding to position D908 of Cas12a, E993 of Cas12a, R1226 of Cas12a, or D1263 of Cas12a (e.g., as shown in Table 5), or a combination thereof.

330. A ZWGD Type V Cas guide RNA (gRNA) molecule.

331. A ZJHK Type V Cas guide RNA (gRNA) molecule.

332. A ZIKV Type V Cas guide RNA (gRNA) molecule.

333. A ZZFT Type V Cas guide RNA (gRNA) molecule.

334. A YYAN Type V Cas guide RNA (gRNA) molecule.

335. A ZZGY Type V Cas guide RNA (gRNA) molecule.

336. A ZKBG Type V Cas guide RNA (gRNA) molecule.

337. A ZZKD Type V Cas guide RNA (gRNA) molecule.

338. A ZXPB Type V Cas guide RNA (gRNA) molecule.

339. A ZPPX Type V Cas guide RNA (gRNA) molecule.

340. A ZXHQ Type V Cas guide RNA (gRNA) molecule.

341. A ZQKH Type V Cas guide RNA (gRNA) molecule.

342. A ZRGM Type V Cas guide RNA (gRNA) molecule.

343. A ZTAE Type V Cas guide RNA (gRNA) molecule.

344. A ZSQQ Type V Cas guide RNA (gRNA) molecule.

345. A ZSYN Type V Cas guide RNA (gRNA) molecule.

346. A ZRBH Type V Cas guide RNA (gRNA) molecule.

347. A ZWPU Type V Cas guide RNA (gRNA) molecule.

348. A ZZQE Type V Cas guide RNA (gRNA) molecule.

349. A ZRXE Type V Cas guide RNA (gRNA) molecule.

350. The gRNA of any one of embodiments 330 to 349, which is a gRNA for editing a human B2M gene.

351. The gRNA of any one of embodiments 330 to 349, which is a gRNA for editing a human TRAC gene.

352. The gRNA of any one of embodiments 330 to 349, which is a gRNA for editing a human PD1 gene.

353. The gRNA of any one of embodiments 330 to 349, which is a gRNA for editing a human AAVS1 genomic sequence.

354. The gRNA of any one of embodiments 330 to 349, which is a gRNA for editing a human EMX1 gene.

355. The gRNA of any one of embodiments 330 to 349, which is a gRNA for editing a human BCL11A gene.

356. The gRNA of any one of embodiments 330 to 349, which is a gRNA for editing a human PCSK9 gene.

357. The gRNA of any one of embodiments 330 to 349, which is a gRNA for editing a human VEGF gene.

358. The gRNA of any one of embodiments 330 to 349, which is a gRNA for editing a human Match6 genomic sequence.

359. A guide RNA (gRNA) molecule for editing a human B2M gene comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 164-168 and 181-183.

360. A guide RNA (gRNA) molecule for editing a human TRAC gene comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 169-173 and 184-192.

361. A guide RNA (gRNA) molecule for editing a human PD1 gene comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 174-177.

362. A guide RNA (gRNA) molecule for editing a human AAVS1 genomic sequence comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 193-196.

363. A guide RNA (gRNA) molecule for editing a human EMX1 genomic sequence comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 197-198.

364. A guide RNA (gRNA) molecule for editing a human BCL11A genomic sequence comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 199-202.

365. A guide RNA (gRNA) molecule for editing a human PCSK9 genomic sequence comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 203-204.

366. A guide RNA (gRNA) molecule for editing a human VEGF genomic sequence comprising a spacer whose nucleotide sequence comprises 15 or more consecutive nucleotides of a reference sequence or comprises a nucleotide sequence that is at least 85% identical to the reference sequence, wherein the reference sequence is SEQ ID NO:205.

367. A guide RNA (gRNA) molecule for editing a human Match6 genomic sequence comprising a spacer whose nucleotide sequence comprises 15 or more consecutive nucleotides of a reference sequence or comprises a nucleotide sequence that is at least 85% identical to the reference sequence, wherein the reference sequence is selected from SEQ ID NOs: 206-210.

368. The gRNA of any one of embodiments 353 to 367, which comprises a spacer that is 15 to 30 nucleotides in length.

369. The gRNA of embodiment 368, wherein the spacer is 18 to 30 nucleotides in length.

370. The gRNA of embodiment 368, wherein the spacer is 20 to 28 nucleotides in length.

371. The gRNA of embodiment 368, wherein the spacer is 22 to 26 nucleotides in length.

372. The gRNA of embodiment 368, wherein the spacer is 23 to 25 nucleotides in length.

373. The gRNA of embodiment 368, wherein the spacer is 22 to 25 nucleotides in length.

374. The gRNA of embodiment 368, wherein the spacer is 15 to 25 nucleotides in length.

375. The gRNA of embodiment 368, wherein the spacer is 16 to 24 nucleotides in length.

376. The gRNA of embodiment 368, wherein the spacer is 17 to 23 nucleotides in length.

377. The gRNA of embodiment 368, wherein the spacer is 18 to 22 nucleotides in length.

378. The gRNA of embodiment 368, wherein the spacer is 19 to 21 nucleotides in length.

379. The gRNA of embodiment 368, wherein the spacer is 25 nucleotides in length.

380. The gRNA of embodiment 368, wherein the spacer is 24 nucleotides in length.

381. The gRNA of embodiment 368, wherein the spacer is 23 nucleotides in length.

382. The gRNA of embodiment 368, wherein the spacer is 22 nucleotides in length.

383. The gRNA of embodiment 368, wherein the spacer is 21 nucleotides in length.

384. The gRNA of embodiment 368, wherein the spacer is 20 nucleotides in length.

385. The gRNA of any one of embodiments 359 to 384, wherein the spacer comprises 16 or more consecutive nucleotides of the reference sequence.

386. The gRNA of any one of embodiments 359 to 384, wherein the spacer comprises 17 or more consecutive nucleotides of the reference sequence.

387. The gRNA of any one of embodiments 359 to 384, wherein the spacer comprises 18 or more consecutive nucleotides of the reference sequence.

388. The gRNA of any one of embodiments 359 to 384, wherein the spacer comprises 19 or more consecutive nucleotides of the reference sequence.

389. The gRNA of any one of embodiments 359 to 384, wherein the spacer comprises 20 or more consecutive nucleotides of the reference sequence.

390. The gRNA of any one of embodiments 359 to 384, wherein the spacer comprises 21 or more consecutive nucleotides of the reference sequence.

391. The gRNA of any one of embodiments 359 to 384, wherein the spacer comprises 22 or more consecutive nucleotides of the reference sequence.

392. The gRNA of any one of embodiments 359 to 384, wherein the spacer comprises 23 consecutive nucleotides of the reference sequence.

393. The gRNA of any one of embodiments 359 to 384, wherein the spacer comprises a nucleotide sequence that is at least 90% identical to the reference sequence.

394. The gRNA of embodiment 393, wherein the spacer comprises a nucleotide sequence that is at least 95% identical to the reference sequence.

395. The gRNA of any one of embodiments 359 to 384, wherein the spacer comprises a nucleotide sequence that has one mismatch relative to the reference sequence.

396. The gRNA of any one of embodiments 359 to 384, wherein the spacer comprises a nucleotide sequence that has two mismatches relative to the reference sequence.

397. The gRNA of any one of embodiments 359 to 367, wherein the spacer comprises the reference sequence.

398. The gRNA of any one of embodiments 359 and 368 to 397 when depending from embodiment 359, wherein the reference sequence is SEQ ID NO: 164.

399. The gRNA of any one of embodiments 359 and 368 to 397 when depending from embodiment 359, wherein the reference sequence is SEQ ID NO: 165.

400. The gRNA of any one of embodiments 359 and 368 to 397 when depending from embodiment 359, wherein the reference sequence is SEQ ID NO: 166.

401. The gRNA of any one of embodiments 359 and 368 to 397 when depending from embodiment 359, wherein the reference sequence is SEQ ID NO: 167.

402. The gRNA of any one of embodiments 359 and 368 to 397 when depending from embodiment 359, wherein the reference sequence is SEQ ID NO: 168.

403. The gRNA of any one of embodiments 359 and 368 to 397 when depending from embodiment 359, wherein the reference sequence is SEQ ID NO: 181.

404. The gRNA of any one of embodiments 359 and 368 to 397 when depending from embodiment 359, wherein the reference sequence is SEQ ID NO: 182.

405. The gRNA of any one of embodiments 359 and 368 to 397 when depending from embodiment 359, wherein the reference sequence is SEQ ID NO: 183.

406. The gRNA of any one of embodiments 360 and 368 to 397 when depending from embodiment 360, wherein the reference sequence is SEQ ID NO: 169.

407. The gRNA of any one of embodiments 360 and 368 to 397 when depending from embodiment 360, wherein the reference sequence is SEQ ID NO: 170.

408. The gRNA of any one of embodiments 360 and 368 to 397 when depending from embodiment 360, wherein the reference sequence is SEQ ID NO: 171.

409. The gRNA of any one of embodiments 360 and 368 to 397 when depending from embodiment 360, wherein the reference sequence is SEQ ID NO: 172.

410. The gRNA of any one of embodiments 360 and 368 to 397 when depending from embodiment 360, wherein the reference sequence is SEQ ID NO: 173.

411. The gRNA of any one of embodiments 360 and 368 to 397 when depending from embodiment 360, wherein the reference sequence is SEQ ID NO: 184.

412. The gRNA of any one of embodiments 360 and 368 to 397 when depending from embodiment 360, wherein the reference sequence is SEQ ID NO: 185.

413. The gRNA of any one of embodiments 360 and 368 to 397 when depending from embodiment 360, wherein the reference sequence is SEQ ID NO: 186.

414. The gRNA of any one of embodiments 360 and 368 to 397 when depending from embodiment 360, wherein the reference sequence is SEQ ID NO: 187.

415. The gRNA of any one of embodiments 360 and 368 to 397 when depending from embodiment 360, wherein the reference sequence is SEQ ID NO: 188.

416. The gRNA of any one of embodiments 360 and 368 to 397 when depending from embodiment 360, wherein the reference sequence is SEQ ID NO: 189.

417. The gRNA of any one of embodiments 360 and 368 to 397 when depending from embodiment 360, wherein the reference sequence is SEQ ID NO: 190.

418. The gRNA of any one of embodiments 360 and 368 to 397 when depending from embodiment 360, wherein the reference sequence is SEQ ID NO: 191.

419. The gRNA of any one of embodiments 360 and 368 to 397 when depending from embodiment 360, wherein the reference sequence is SEQ ID NO: 192.

420. The gRNA of any one of embodiments 361 and 368 to 397 when depending from embodiment 361, wherein the reference sequence is SEQ ID NO: 174.

421. The gRNA of any one of embodiments 361 and 368 to 397 when depending from embodiment 361, wherein the reference sequence is SEQ ID NO: 175.

422. The gRNA of any one of embodiments 361 and 368 to 397 when depending from embodiment 361, wherein the reference sequence is SEQ ID NO: 176.

423. The gRNA of any one of embodiments 361 and 368 to 397 when depending from embodiment 361, wherein the reference sequence is SEQ ID NO: 177.

424. The gRNA of any one of embodiments 362 and 368 to 397 when depending from embodiment 362, wherein the reference sequence is SEQ ID NO: 193.

425. The gRNA of any one of embodiments 362 and 368 to 397 when depending from embodiment 362, wherein the reference sequence is SEQ ID NO: 194.

426. The gRNA of any one of embodiments 362 and 368 to 397 when depending from embodiment 362, wherein the reference sequence is SEQ ID NO: 195.

427. The gRNA of any one of embodiments 362 and 368 to 397 when depending from embodiment 362, wherein the reference sequence is SEQ ID NO: 196.

428. The gRNA of any one of embodiments 363 and 368 to 397 when depending from embodiment 363, wherein the reference sequence is SEQ ID NO: 197.

429. The gRNA of any one of embodiments 363 and 368 to 397 when depending from embodiment 363, wherein the reference sequence is SEQ ID NO: 198.

430. The gRNA of any one of embodiments 364 and 368 to 397 when depending from embodiment 364, wherein the reference sequence is SEQ ID NO: 199.

431. The gRNA of any one of embodiments 364 and 368 to 397 when depending from embodiment 364, wherein the reference sequence is SEQ ID NO:200.

432. The gRNA of any one of embodiments 364 and 368 to 397 when depending from embodiment 364, wherein the reference sequence is SEQ ID NO:201.

433. The gRNA of any one of embodiments 364 and 368 to 397 when depending from embodiment 364, wherein the reference sequence is SEQ ID NO:202.

434. The gRNA of any one of embodiments 365 and 368 to 397 when depending from embodiment 365, wherein the reference sequence is SEQ ID NO:203.

435. The gRNA of any one of embodiments 365 and 368 to 397 when depending from embodiment 365, wherein the reference sequence is SEQ ID NO:204.

436. The gRNA of any one of embodiments 366 and 368 to 397 when depending from embodiment 366, wherein the reference sequence is SEQ ID NO:205.

437. The gRNA of any one of embodiments 367 and 368 to 397 when depending from embodiment 367, wherein the reference sequence is SEQ ID NO:206.

438. The gRNA of any one of embodiments 367 and 368 to 397 when depending from embodiment 367, wherein the reference sequence is SEQ ID NO:207.

439. The gRNA of any one of embodiments 367 and 368 to 397 when depending from embodiment 367, wherein the reference sequence is SEQ ID NO:208.

440. The gRNA of any one of embodiments 367 and 368 to 397 when depending from embodiment 367, wherein the reference sequence is SEQ ID NO:209.

441. The gRNA of any one of embodiments 367 and 368 to 397 when depending from embodiment 367, wherein the reference sequence is SEQ ID NO:210.

442. A gRNA comprising a spacer and a crRNA scaffold, which is optionally a gRNA according to any one of embodiments 330 to 441, wherein:
(a) the spacer is positioned 3′ to the crRNA scaffold; and
(b) the nucleotide sequence of the crRNA scaffold comprises a nucleotide sequence that is at least 50% identical to a reference scaffold sequence, wherein the reference scaffold sequence is selected from SEQ ID NOS: 144-163 and 211-213.

443. A gRNA comprising a means for binding a target mammalian genomic sequence and a crRNA scaffold, optionally wherein the means for binding a target mammalian genomic sequence is a spacer, wherein:
(a) the means for binding a target genomic sequence is positioned 3′ to the crRNA scaffold; and
(b) the nucleotide sequence of the crRNA scaffold comprises a nucleotide sequence that is at least 50% identical to a reference scaffold sequence, wherein the reference scaffold sequence is selected from SEQ ID NOS: 144-163 and 211-213.

444. The gRNA of embodiment 442 or 443, wherein the crRNA scaffold comprises a nucleotide sequence that is at least 55% identical to the reference scaffold sequence.

445. The gRNA of embodiment 444, wherein the crRNA scaffold comprises a nucleotide sequence that is at least 60% identical to the reference scaffold sequence.

446. The gRNA of embodiment 444, wherein the crRNA scaffold comprises a nucleotide sequence that is at least 65% identical to the reference scaffold sequence.

447. The gRNA of embodiment 444, wherein the crRNA scaffold comprises a nucleotide sequence that is at least 70% identical to the reference scaffold sequence.

448. The gRNA of embodiment 444, wherein the crRNA scaffold comprises a nucleotide sequence that is at least 75% identical to the reference scaffold sequence.

449. The gRNA of embodiment 444, wherein the crRNA scaffold comprises a nucleotide sequence that is at least 80% identical to the reference scaffold sequence.

450. The gRNA of embodiment 444, wherein the crRNA scaffold comprises a nucleotide sequence that is at least 85% identical to the reference scaffold sequence.

451. The gRNA of embodiment 444, wherein the crRNA scaffold comprises a nucleotide sequence that is at least 90% identical to the reference scaffold sequence.

452. The gRNA of embodiment 444, wherein the crRNA scaffold comprises a nucleotide sequence that is at least 95% identical to the reference scaffold sequence.

453. The gRNA of embodiment 444, wherein the crRNA scaffold comprises a nucleotide sequence that is at least 96% identical to the reference scaffold sequence.

454. The gRNA of embodiment 444, wherein the crRNA scaffold comprises a nucleotide sequence that is at least 97% identical to the reference scaffold sequence.

455. The gRNA of embodiment 444, wherein the crRNA scaffold comprises a nucleotide sequence that is at least 98% identical to the reference scaffold sequence.

456. The gRNA of embodiment 444, wherein the crRNA scaffold comprises a nucleotide sequence that is at least 99% identical to the reference scaffold sequence.

457. The gRNA of embodiment 444, wherein the crRNA scaffold comprises a nucleotide sequence that has no more than 5 nucleotide mismatches with the reference scaffold sequence.

458. The gRNA of embodiment 444, wherein the crRNA scaffold comprises a nucleotide sequence that has no more than 4 nucleotide mismatches with the reference scaffold sequence.

459. The gRNA of embodiment 444, wherein the crRNA scaffold comprises a nucleotide sequence that has no more than 3 nucleotide mismatches with the reference scaffold sequence.

460. The gRNA of embodiment 444, wherein the crRNA scaffold comprises a nucleotide sequence that has no more than 2 nucleotide mismatches with the reference scaffold sequence.

461. The gRNA of embodiment 444, wherein the crRNA scaffold comprises a nucleotide sequence that has no more than 1 nucleotide mismatches with the reference scaffold sequence.

462. The gRNA of embodiment 442 or embodiment 443, wherein the crRNA scaffold comprises a nucleotide sequence that is 100% identical to the reference scaffold sequence.

463. The gRNA of any one of embodiments 442 to 462, wherein the reference scaffold sequence is SEQ ID NO: 144.

464. The gRNA of any one of embodiments 442 to 462, wherein the reference scaffold sequence is SEQ ID NO: 145.

465. The gRNA of any one of embodiments 442 to 462, wherein the reference scaffold sequence is SEQ ID NO: 146.

466. The gRNA of any one of embodiments 442 to 462, wherein the reference scaffold sequence is SEQ ID NO: 147.

467. The gRNA of any one of embodiments 442 to 462, wherein the reference scaffold sequence is SEQ ID NO: 148.

468. The gRNA of any one of embodiments 442 to 462, wherein the reference scaffold sequence is SEQ ID NO: 149.

469. The gRNA of any one of embodiments 442 to 462, wherein the reference scaffold sequence is SEQ ID NO: 150.

470. The gRNA of any one of embodiments 442 to 462, wherein the reference scaffold sequence is SEQ ID NO: 151.

471. The gRNA of any one of embodiments 442 to 462, wherein the reference scaffold sequence is SEQ ID NO: 152.

472. The gRNA of any one of embodiments 442 to 462, wherein the reference scaffold sequence is SEQ ID NO: 153.

473. The gRNA of any one of embodiments 442 to 462, wherein the reference scaffold sequence is SEQ ID NO: 154.

474. The gRNA of any one of embodiments 442 to 462, wherein the reference scaffold sequence is SEQ ID NO: 155.

475. The gRNA of any one of embodiments 442 to 462, wherein the reference scaffold sequence is SEQ ID NO: 156.

476. The gRNA of any one of embodiments 442 to 462, wherein the reference scaffold sequence is SEQ ID NO: 157.

477. The gRNA of any one of embodiments 442 to 462, wherein the reference scaffold sequence is SEQ ID NO: 158.

478. The gRNA of any one of embodiments 442 to 462, wherein the reference scaffold sequence is SEQ ID NO: 159.

479. The gRNA of any one of embodiments 442 to 462, wherein the reference scaffold sequence is SEQ ID NO: 160.

480. The gRNA of any one of embodiments 442 to 462, wherein the reference scaffold sequence is SEQ ID NO: 161.

481. The gRNA of any one of embodiments 442 to 462, wherein the reference scaffold sequence is SEQ ID NO: 162.

482. The gRNA of any one of embodiments 442 to 462, wherein the reference scaffold sequence is SEQ ID NO: 163.

483. The gRNA of any one of embodiments 442 to 462, wherein the reference scaffold sequence is SEQ ID NO:211.

484. The gRNA of any one of embodiments 442 to 462, wherein the reference scaffold sequence is SEQ ID NO:212.

485. The gRNA of any one of embodiments 442 to 462, wherein the reference scaffold sequence is SEQ ID NO:213.

486. The gRNA of any one of embodiments 442 to 485, wherein the gRNA comprises 1 to 8 uracils at its 3′ end.

487. The gRNA of embodiment 486, wherein the gRNA comprises 1 uracil at its 3′ end.

488. The gRNA of embodiment 486, wherein the gRNA comprises 2 uracils at its 3′ end.

489. The gRNA of embodiment 486, wherein the gRNA comprises 3 uracils at its 3′ end.

490. The gRNA of embodiment 486, wherein the gRNA comprises 4 uracils at its 3′ end.

491. The gRNA of embodiment 486, wherein the gRNA comprises 5 uracils at its 3′ end.

492. The gRNA of embodiment 486, wherein the gRNA comprises 6 uracils at its 3′ end.

493. The gRNA of embodiment 486, wherein the gRNA comprises 7 uracils at its 3′ end.

494. The gRNA of embodiment 486, wherein the gRNA comprises 8 uracils at its 3′ end.

495. The gRNA of any one of embodiments 442 to 494, which comprises a 5′ guanine.

496. The gRNA of any one of embodiments 442 to 495, wherein the nucleotide sequence of the spacer is partially or fully complementary to a target mammalian genomic sequence.

497. The gRNA of embodiment 496, wherein the target mammalian genomic sequence is downstream of a protospacer adjacent motif (PAM) sequence in the non-target strand recognized by a Type V Cas protein, optionally wherein the Type V Cas protein is a Type V Cas protein according to any one of embodiments 1 to 329.

498. The gRNA of embodiment 497, wherein the PAM sequence is TTN.

499. The gRNA of embodiment 497, wherein the PAM sequence is TTTN, e.g., TTTA, TTTT, TTTG, or TTTC.

500. The gRNA of embodiment 497, wherein the PAM sequence is TTTR.

501. The gRNA of embodiment 497, wherein the PAM sequence is YTTN, e.g., CTTC or CTTG.

502. The gRNA of embodiment 497, wherein the PAM sequence is YTTV.

503. The gRNA of embodiment 497, wherein the PAM sequence is NTTV.

504. The gRNA of embodiment 497, wherein the PAM sequence is VTTV, e.g., ATTA, or GTTA, or ATTC.

505. The gRNA of embodiment 497, wherein the PAM sequence is NCTV.

506. The gRNA of embodiment 497, wherein the PAM sequence is DTTN.

507. The gRNA of embodiment 497, wherein the PAM sequence is DTDN.

508. The gRNA of embodiment 497, wherein the PAM sequence is TTTT.

509. The gRNA of embodiment 497, wherein the PAM sequence is NYYN.

510. The gRNA of embodiment 497, wherein the PAM sequence is NTTN.

511. A gRNA comprising a spacer whose sequence comprises SEQ ID NO: 164.

512. A gRNA comprising a spacer whose sequence comprises SEQ ID NO: 165.

513. A gRNA comprising a spacer whose sequence comprises SEQ ID NO: 166.

514. A gRNA comprising a spacer whose sequence comprises SEQ ID NO: 167.

515. A gRNA comprising a spacer whose sequence comprises SEQ ID NO: 168.

516. A gRNA comprising a spacer whose sequence comprises SEQ ID NO: 169.

517. A gRNA comprising a spacer whose sequence comprises SEQ ID NO: 170.

518. A gRNA comprising a spacer whose sequence comprises SEQ ID NO: 171.

519. A gRNA comprising a spacer whose sequence comprises SEQ ID NO: 172.

520. A gRNA comprising a spacer whose sequence comprises SEQ ID NO: 173.

521. A gRNA comprising a spacer whose sequence comprises SEQ ID NO: 174.

522. A gRNA comprising a spacer whose sequence comprises SEQ ID NO: 175.

523. A gRNA comprising a spacer whose sequence comprises SEQ ID NO: 176.

524. A gRNA comprising a spacer whose sequence comprises SEQ ID NO: 177.

525. A gRNA comprising a spacer whose sequence comprises SEQ ID NO: 181.

526. A gRNA comprising a spacer whose sequence comprises SEQ ID NO: 182.

527. A gRNA comprising a spacer whose sequence comprises SEQ ID NO: 183.

528. A gRNA comprising a spacer whose sequence comprises SEQ ID NO: 184.

529. A gRNA comprising a spacer whose sequence comprises SEQ ID NO: 185.

530. A gRNA comprising a spacer whose sequence comprises SEQ ID NO: 186.

531. A gRNA comprising a spacer whose sequence comprises SEQ ID NO: 187.

532. A gRNA comprising a spacer whose sequence comprises SEQ ID NO:188.

533. A gRNA comprising a spacer whose sequence comprises SEQ ID NO: 189.

534. A gRNA comprising a spacer whose sequence comprises SEQ ID NO: 190.

535. A gRNA comprising a spacer whose sequence comprises SEQ ID NO: 191.

536. A gRNA comprising a spacer whose sequence comprises SEQ ID NO: 192.

537. A gRNA comprising a spacer whose sequence comprises SEQ ID NO: 193.

538. A gRNA comprising a spacer whose sequence comprises SEQ ID NO: 194.

539. A gRNA comprising a spacer whose sequence comprises SEQ ID NO: 195.

540. A gRNA comprising a spacer whose sequence comprises SEQ ID NO: 196.

541. A gRNA comprising a spacer whose sequence comprises SEQ ID NO: 197.

542. A gRNA comprising a spacer whose sequence comprises SEQ ID NO: 198.

543. A gRNA comprising a spacer whose sequence comprises SEQ ID NO: 199.

544. A gRNA comprising a spacer whose sequence comprises SEQ ID NO:200.

545. A gRNA comprising a spacer whose sequence comprises SEQ ID NO:201.

546. A gRNA comprising a spacer whose sequence comprises SEQ ID NO:202.

547. A gRNA comprising a spacer whose sequence comprises SEQ ID NO:203.

548. A gRNA comprising a spacer whose sequence comprises SEQ ID NO:204.

549. A gRNA comprising a spacer whose sequence comprises SEQ ID NO:205.

550. A gRNA comprising a spacer whose sequence comprises SEQ ID NO:206.

551. A gRNA comprising a spacer whose sequence comprises SEQ ID NO:207.

552. A gRNA comprising a spacer whose sequence comprises SEQ ID NO:208.

553. A gRNA comprising a spacer whose sequence comprises SEQ ID NO:209.

554. A gRNA comprising a spacer whose sequence comprises SEQ ID NO:210.

555. A gRNA comprising a spacer whose sequence comprises SEQ ID NO:211.

556. The gRNA of any one of embodiments 511 to 555, wherein the spacer is positioned 3′ to a scaffold whose sequence comprises the sequence of SEQ ID NO: 144.

557. The gRNA of any one of embodiments 511 to 555, wherein the spacer is positioned 3′ to a scaffold whose sequence comprises the sequence of SEQ ID NO:145.

558. The gRNA of any one of embodiments 511 to 555, wherein the spacer is positioned 3′ to a scaffold whose sequence comprises the sequence of SEQ ID NO: 146.

559. The gRNA of any one of embodiments 511 to 555, wherein the spacer is positioned 3′ to a scaffold whose sequence comprises the sequence of SEQ ID NO: 147.

560. The gRNA of any one of embodiments 511 to 555, wherein the spacer is positioned 3′ to a scaffold whose sequence comprises the sequence of SEQ ID NO: 148.

561. The gRNA of any one of embodiments 511 to 555, wherein the spacer is positioned 3′ to a scaffold whose sequence comprises the sequence of SEQ ID NO: 149.

562. The gRNA of any one of embodiments 511 to 555, wherein the spacer is positioned 3′ to a scaffold whose sequence comprises the sequence of SEQ ID NO:150.

563. The gRNA of any one of embodiments 511 to 555, wherein the spacer is positioned 3′ to a scaffold whose sequence comprises the sequence of SEQ ID NO: 151.

564. The gRNA of any one of embodiments 511 to 555, wherein the spacer is positioned 3′ to a scaffold whose sequence comprises the sequence of SEQ ID NO: 152.

565. The gRNA of any one of embodiments 511 to 555, wherein the spacer is positioned 3′ to a scaffold whose sequence comprises the sequence of SEQ ID NO: 153.

566. The gRNA of any one of embodiments 511 to 555, wherein the spacer is positioned 3′ to a scaffold whose sequence comprises the sequence of SEQ ID NO: 154.

567. The gRNA of any one of embodiments 511 to 555, wherein the spacer is positioned 3′ to a scaffold whose sequence comprises the sequence of SEQ ID NO: 155.

568. The gRNA of any one of embodiments 511 to 555, wherein the spacer is positioned 3′ to a scaffold whose sequence comprises the sequence of SEQ ID NO: 156.

569. The gRNA of any one of embodiments 511 to 555, wherein the spacer is positioned 3′ to a scaffold whose sequence comprises the sequence of SEQ ID NO: 157.

570. The gRNA of any one of embodiments 511 to 555, wherein the spacer is positioned 3′ to a scaffold whose sequence comprises the sequence of SEQ ID NO: 158.

571. The gRNA of any one of embodiments 511 to 555, wherein the spacer is positioned 3′ to a scaffold whose sequence comprises the sequence of SEQ ID NO: 159.

572. The gRNA of any one of embodiments 511 to 555, wherein the spacer is positioned 3′ to a scaffold whose sequence comprises the sequence of SEQ ID NO: 160.

573. The gRNA of any one of embodiments 511 to 555, wherein the spacer is positioned 3′ to a scaffold whose sequence comprises the sequence of SEQ ID NO: 161.

574. The gRNA of any one of embodiments 511 to 555, wherein the spacer is positioned 3′ to a scaffold whose sequence comprises the sequence of SEQ ID NO: 162.

575. The gRNA of any one of embodiments 511 to 555, wherein the spacer is positioned 3′ to a scaffold whose sequence comprises the sequence of SEQ ID NO: 163.

576. The gRNA of any one of embodiments 511 to 555, wherein the spacer is positioned 3′ to a scaffold whose sequence comprises the sequence of SEQ ID NO:211

577. The gRNA of any one of embodiments 511 to 555, wherein the spacer is positioned 3′ to a scaffold whose sequence comprises the sequence of SEQ ID NO:212.

578. The gRNA of any one of embodiments 511 to 555, wherein the spacer is positioned 3′ to a scaffold whose sequence comprises the sequence of SEQ ID NO:213.

579. A system comprising the Type V Cas protein of any one of embodiments 1 to 329 and a guide RNA (gRNA) comprising a spacer sequence, optionally wherein the gRNA is a gRNA according to any one of embodiments 330 to 578.

580. A system comprising the Type V Cas protein of any one of embodiments 1 to 329 and a means for targeting the Type V Cas protein to a target genomic sequence, optionally wherein the means for targeting the Type V Cas protein to a target genomic sequence is a guide RNA (gRNA) molecule, optionally as described in in any one of embodiments 330 to 578, optionally wherein the gRNA molecule comprises a spacer partially or fully complementary to a target mammalian genomic sequence.

581. The system of embodiment 580, wherein the spacer sequence is partially or fully complementary to a target mammalian genomic sequence.

582. The system of any one of embodiments 580 to 581, wherein the target mammalian genomic sequence is a human genomic sequence.

583. The system of embodiment 582, wherein the target mammalian genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence. 584. The system of embodiment 582, wherein the target mammalian genomic sequence is a RHO genomic sequence.

585. The system of embodiment 582, wherein the target mammalian genomic sequence is a TRAC genomic sequence.

586. The system of embodiment 582, wherein the target mammalian genomic sequence is a B2M genomic sequence.

587. The system of embodiment 582, wherein the target mammalian genomic sequence is a PD1 genomic sequence.

588. The system of embodiment 582, wherein the target mammalian genomic sequence is an AAVS 1 genomic sequence.

589. The system of embodiment 582, wherein the target mammalian genomic sequence is an EMX1 genomic sequence.

590. The system of embodiment 582, wherein the target mammalian genomic sequence is an BCL11A genomic sequence.

591. The system of embodiment 582, wherein the target mammalian genomic sequence is an PCSK9 genomic sequence.

592. The system of embodiment 582, wherein the target mammalian genomic sequence is an VEGFA genomic sequence.

593. The system of embodiment 582, wherein the target mammalian genomic sequence is an Match6 genomic sequence.

594. The system of any one of embodiments 579 to 593, which is a ribonucleoprotein (RNP) comprising the Type V Cas protein complexed to the gRNA or means for targeting the Type V Cas protein to a target genomic sequence.

595. A nucleic acid encoding the Type V Cas protein of any one of embodiments 1 to 329, optionally wherein the nucleotide sequence encoding the Type V Cas protein is operably linked to a promoter that is heterologous to the Type V Cas protein.

596. The nucleic acid of embodiment 595, wherein the nucleotide sequence encoding the Type V Cas protein is codon optimized for expression in human cells.

597. The nucleic acid of embodiment 596, wherein when the reference protein sequence is SEQ ID NO:1 or SEQ ID NO:2, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:5 or SEQ ID NO: 6.

598. The nucleic acid of embodiment 596, wherein when the reference protein sequence is SEQ ID NO:7 or SEQ ID NO:8, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO: 11 or SEQ ID NO: 12.

599. The nucleic acid of embodiment 596, wherein when the reference protein sequence is SEQ ID NO: 13 or SEQ ID NO: 14, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO: 17 or SEQ ID NO: 18.

600. The nucleic acid of embodiment 596, wherein when the reference protein sequence is SEQ ID NO:19 or SEQ ID NO:20, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:23 or SEQ ID NO: 24.

601. The nucleic acid of embodiment 596, wherein when the reference protein sequence is SEQ ID NO:25 or SEQ ID NO:26, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:29 or SEQ ID NO: 30.

602. The nucleic acid of embodiment 596, wherein when the reference protein sequence is SEQ ID NO:31 or SEQ ID NO:32, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:35 or SEQ ID NO: 36.

603. The nucleic acid of embodiment 596, wherein when the reference protein sequence is SEQ ID NO:37 or SEQ ID NO:38, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:41 or SEQ ID NO: 42.

604. The nucleic acid of embodiment 596, wherein when the reference protein sequence is SEQ ID NO:43 or SEQ ID NO:44, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:47 or SEQ ID NO: 48.

605. The nucleic acid of embodiment 596, wherein when the reference protein sequence is SEQ ID NO:49 or SEQ ID NO:50, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:53 or SEQ ID NO: 54.

606. The nucleic acid of embodiment 596, wherein when the reference protein sequence is SEQ ID NO:55 or SEQ ID NO:56, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:59 or SEQ ID NO: 60.

607. The nucleic acid of embodiment 596, wherein when the reference protein sequence is SEQ ID NO:61 or SEQ ID NO:62, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:65 or SEQ ID NO: 66.

608. The nucleic acid of embodiment 596, wherein when the reference protein sequence is SEQ ID NO:67 or SEQ ID NO:68, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:71 or SEQ ID NO: 72.

609. The nucleic acid of embodiment 596, wherein when the reference protein sequence is SEQ ID NO:73 or SEQ ID NO:74, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:77 or SEQ ID NO: 78.

610. The nucleic acid of embodiment 596, wherein when the reference protein sequence is SEQ ID NO:79 or SEQ ID NO:80, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:83 or SEQ ID NO: 84.

611. The nucleic acid of embodiment 596, wherein when the reference protein sequence is SEQ ID NO:85 or SEQ ID NO:86, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:89 or SEQ ID NO: 90.

612. The nucleic acid of embodiment 596, wherein when the reference protein sequence is SEQ ID NO:91 or SEQ ID NO:92, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO:95 or SEQ ID NO: 96.

613. The nucleic acid of embodiment 596, wherein when the reference protein sequence is SEQ ID NO:97 or SEQ ID NO:98, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO: 101 or SEQ ID NO: 102.

614. The nucleic acid of embodiment 596, wherein when the reference protein sequence is SEQ ID NO: 103 or SEQ ID NO: 104, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO: 107 or SEQ ID NO: 108.

615. The nucleic acid of embodiment 596, wherein when the reference protein sequence is SEQ ID NO: 109 or SEQ ID NO: 110, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO: 113 or SEQ ID NO: 114.

616. The nucleic acid of embodiment 596, wherein when the reference protein sequence is SEQ ID NO:115 or SEQ ID NO: 116, the nucleotide sequence encoding the Type V Cas protein comprises a nucleotide sequence that is at least 80%, at least 85%, at least 90%, at least 95%, at least 96%, at least 97%, at least 98%, at least 99%, or 100% identical to the nucleotide sequence of SEQ ID NO: 119 or SEQ ID NO: 120.

617. The nucleic acid of any one of embodiments embodiment 595 to 616, which is a plasmid.

618. The nucleic acid of any one of embodiments embodiment 595 to 616, which is a viral genome.

619. The nucleic acid of embodiment 618, wherein the viral genome is an adeno-associated virus (AAV) genome.

620. The nucleic acid of embodiment 619, wherein the AAV genome is an AAV2, AAV5, AAV7m8, AAV8, AAV9, AAVrh8r, or AAVrh10 genome.

621. The nucleic acid of embodiment 620, wherein the AAV genome is an AAV2 genome.

622. The nucleic acid of embodiment 620, wherein the AAV genome is an AAV5 genome.

623. The nucleic acid of embodiment 620, wherein the AAV genome is an AAV7m8 genome.

624. The nucleic acid of embodiment 620, wherein the AAV genome is an AAV8 genome.

625. The nucleic acid of embodiment 620, wherein the AAV genome is an AAV9 genome.

626. The nucleic acid of embodiment 620, wherein the AAV genome is an AAVrh8r genome.

627. The nucleic acid of embodiment 620, wherein the AAV genome is an AAVrh10 genome.

628. The nucleic acid of any one of embodiments 595 to 627, further encoding a gRNA, optionally wherein the gRNA is a gRNA according to any one of embodiments 330 to 578.

629. A nucleic acid encoding the gRNA of any one of embodiments 330 to 578.

630. The nucleic acid of embodiment 629, which is a plasmid.

631. The nucleic acid of embodiment 629, which is a viral genome.

632. The nucleic acid of embodiment 631, wherein the viral genome is an adeno-associated virus (AAV) genome.

633. The nucleic acid of embodiment 632, wherein the AAV genome is a AAV2, AAV5, AAV7m8, AAV8, AAV9, AAVrh8r, or AAVrh10 genome.

634. The nucleic acid of embodiment 633, wherein the AAV genome is an AAV2 genome.

635. The nucleic acid of embodiment 633, wherein the AAV genome is an AAV5 genome.

636. The nucleic acid of embodiment 633, wherein the AAV genome is an AAV7m8 genome.

637. The nucleic acid of embodiment 633, wherein the AAV genome is an AAV8 genome.

638. The nucleic acid of embodiment 633, wherein the AAV genome is an AAV9 genome.

639. The nucleic acid of embodiment 633, wherein the AAV genome is an AAVrh8r genome.

640. The nucleic acid of embodiment 633, wherein the AAV genome is an AAVrh10 genome.

641. The nucleic acid of any one of embodiments 629 to 640, further encoding a Type V Cas protein, optionally wherein the Type V Cas protein is a Type V Cas protein according to any one of embodiments 1 to 329.

642. A nucleic acid encoding the Type V Cas protein and gRNA of the system of any one of embodiments 579 to 594.

643. The nucleic acid of embodiment 642, wherein the nucleotide sequence encoding the Type V Cas protein is codon optimized for expression in human cells.

644. The nucleic acid of embodiment 642 or embodiment 643, which is a plasmid.

645. The nucleic acid of embodiment 642 or embodiment 643, which is a viral genome.

646. The nucleic acid of embodiment 645, wherein the viral genome is an adeno-associated virus (AAV) genome.

647. The nucleic acid of embodiment 646, wherein the AAV genome is an AAV2, AAV5, AAV7m8, AAV8, AAV9, AAVrh8r, or AAVrh10 genome.

648. The nucleic acid of embodiment 647, wherein the AAV genome is an AAV2 genome.

649. The nucleic acid of embodiment 647, wherein the AAV genome is an AAV5 genome.

650. The nucleic acid of embodiment 647, wherein the AAV genome is an AAV7m8 genome.

651. The nucleic acid of embodiment 647, wherein the AAV genome is an AAV8 genome.

652. The nucleic acid of embodiment 647, wherein the AAV genome is an AAV9 genome.

653. The nucleic acid of embodiment 647, wherein the AAV genome is an AAVrh8r genome.

654. The nucleic acid of embodiment 647, wherein the AAV genome is an AAVrh10 genome.

655. A plurality of nucleic acids comprising separate nucleic acids encoding the Type V Cas protein and gRNA of the system of any one of embodiments 579 to 594.

656. The plurality of nucleic acid of embodiment 655, wherein the separate nucleic acids encoding the Type V Cas protein and gRNA are plasmids.

657. The plurality of nucleic acids of embodiment 655, wherein the separate nucleic acids encoding the Type V Cas protein and gRNA are viral genomes.

658. The plurality of nucleic acids of embodiment 657, wherein the viral genomes are adeno-associated virus (AAV) genomes.

659. The plurality of nucleic acids of embodiment 658, wherein the AAV genomes the encoding the Type V Cas protein and gRNA are independently an AAV2, AAV5, AAV7m8, AAV8, AAV9, AAVrh8r, or AAVrh10 genome.

660. A Type V Cas protein according to any one of embodiments 1 to 329, a gRNA according to any one of embodiments 330 to 578, a system according to of any one of embodiments 579 to 594, a nucleic acid according to any one of embodiments 595 to 654, a plurality of nucleic acids according to of any one of embodiments 655 to 659, particle according to any one of embodiments 672 to 687, or pharmaceutical composition according to embodiment 688 for use in a method of editing a human genomic sequence.

661. The Type V Cas protein, gRNA, system, nucleic acid, a plurality of nucleic acids, particle, or pharmaceutical composition for use according to embodiment 660, wherein the human genomic sequence is a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence.

662. The Type V Cas protein, gRNA, combination of gRNAs, system, nucleic acid, a plurality of nucleic acids, particle, or pharmaceutical composition for use according to embodiment 660, wherein the human genomic sequence is a RHO genomic sequence, optionally wherein the RHO genomic sequence has a pathogenic mutation.

663. The Type V Cas protein, gRNA, combination of gRNAs, system, nucleic acid, a plurality of nucleic acids, particle, or pharmaceutical composition for use according to embodiment 660, wherein the human genomic sequence is a TRAC genomic sequence, optionally wherein the human genomic sequence is in a T cell.

664. The Type V Cas protein, gRNA, combination of gRNAs, system, nucleic acid, a plurality of nucleic acids, particle, or pharmaceutical composition for use according to embodiment 660, wherein the human genomic sequence is a B2M genomic sequence, optionally wherein the human genomic sequence is in a T cell.

665. The Type V Cas protein, gRNA, combination of gRNAs, system, nucleic acid, a plurality of nucleic acids, particle, or pharmaceutical composition for use according to embodiment 660, wherein the human genomic sequence is a PD1 genomic sequence, optionally wherein the human genomic sequence is in a T cell.

666. The Type V Cas protein, gRNA, combination of gRNAs, system, nucleic acid, a plurality of nucleic acids, particle, or pharmaceutical composition for use according to embodiment 660, wherein the human genomic sequence is a LAG3 genomic sequence, optionally wherein the human genomic sequence is in a T cell.

667. The Type V Cas protein, gRNA, combination of gRNAs, system, nucleic acid, a plurality of nucleic acids, particle, or pharmaceutical composition for use according to embodiment 660, wherein the human genomic sequence is a AAVS1 genomic sequence, optionally wherein the human genomic sequence is in a T cell.

668. The Type V Cas protein, gRNA, combination of gRNAs, system, nucleic acid, a plurality of nucleic acids, particle, or pharmaceutical composition for use according to embodiment 660, wherein the human genomic sequence is an EMX1 genomic sequence.

669. The Type V Cas protein, gRNA, combination of gRNAs, system, nucleic acid, a plurality of nucleic acids, particle, or pharmaceutical composition for use according to embodiment 660, wherein the human genomic sequence is a BCL11A genomic sequence.

670. The Type V Cas protein, gRNA, combination of gRNAs, system, nucleic acid, a plurality of nucleic acids, particle, or pharmaceutical composition for use according to embodiment 660, wherein the human genomic sequence is a PCSK9 genomic sequence.

671. The Type V Cas protein, gRNA, combination of gRNAs, system, nucleic acid, a plurality of nucleic acids, particle, or pharmaceutical composition for use according to embodiment 660, wherein the human genomic sequence is a Match6 genomic sequence.

672. A particle comprising a Type V Cas protein according to any one of embodiments 1 to 329, a gRNA according to any one of embodiments 330 to 578, a system according to of any one of embodiments 579 to 594, a nucleic acid according to any one of embodiments 595 to 654, or a plurality of nucleic acids according to of any one of embodiments 655 to 659.

673. The particle of embodiment 667, which is a lipid nanoparticle, a vesicle, a gold nanoparticle, a viral-like particle (VLP) or a viral particle.

674. The particle of embodiment 673, which is a lipid nanoparticle.

675. The particle of embodiment 673, which is a vesicle.

676. The particle of embodiment 673, which is a gold nanoparticle.

677. The particle of embodiment 673, which is a viral-like particle (VLP).

678. The particle of embodiment 673, which is a viral particle.

679. The particle of embodiment 677, which is an adeno-associated virus (AAV) particle.

680. The particle of embodiment 679, wherein the AAV particle is an AAV2, AAV5, AAV7m8,

AAV8, AAV9, AAVrh8r, or AAVrh10 particle.

681. The particle of embodiment 680, wherein the AAV particle is an AAV2 particle.

682. The particle of embodiment 680, wherein the AAV particle is an AAV5 particle.

683. The particle of embodiment 680, wherein the AAV particle is an AAV7m8 particle.

684. The particle of embodiment 680, wherein the AAV particle is an AAV8 particle.

685. The particle of embodiment 680, wherein the AAV particle is an AAV9 particle.

686. The particle of embodiment 680, wherein the AAV particle is an AAVrh8r particle.

687. The particle of embodiment 680, wherein the AAV particle is an AAVrh10 particle.

688. A pharmaceutical composition comprising a Type V Cas protein according to any one of embodiments 1 to 329, a gRNA according to any one of embodiments 330 to 578, a system according to of any one of embodiments 579 to 594, a nucleic acid according to any one of embodiments 595 to 654, a plurality of nucleic acids according to of any one of embodiments 655 to 659, or a particle according to any one of embodiments 667 to 687 and at least one pharmaceutically acceptable excipient.

689. A cell comprising a Type V Cas protein according to any one of embodiments 1 to 329, a gRNA according to any one of embodiments 330 to 578, a system according to of any one of embodiments 579 to 594, a nucleic acid according to any one of embodiments 595 to 654, a plurality of nucleic acids according to of any one of embodiments 655 to 659, or a particle according to any one of embodiments 667 to 687.

690. The cell of embodiment 689, which is a human cell.

691. The cell of embodiment 689 or embodiment 690, wherein the cell is a hematopoietic progenitor cell.

692. The cell of any one of embodiments 689 to 691, which is a stem cell.

693. The cell of embodiment 692, wherein the stem cell is a hematopoietic stem cell (HSC), a pluripotent stem cell, or an induced pluripotent stem cell (iPS).

694. The cell of embodiment 693, wherein the stem cell is an embryonic stem cell.

695. The cell of embodiment 689 or embodiment 690, which is a T cell.

696. The cell of embodiment 689 or embodiment 690, which is a retinal cell.

697. The cell of embodiment 689 or embodiment 690, which is a photoreceptor cell.

698. The cell of any one of embodiments 689 to 697, which is an ex vivo cell.

699. A population of cells according to any one of embodiments 689 to 698.

700. A method for altering a cell, the method comprising contacting the cell with a Type V Cas protein according to any one of embodiments 1 to 329, a gRNA according to any one of embodiments 330 to 578, a system according to of any one of embodiments 579 to 594, a nucleic acid according to any one of embodiments 595 to 654, a plurality of nucleic acids according to of any one of embodiments 655 to 659, or a particle according to any one of embodiments 667 to 687, or a pharmaceutical composition according to embodiment 688.

701. The method of embodiment 700, which comprises contacting the cell with the Type V Cas protein of any one of embodiments 1 to 329.

702. The method of embodiment 700, which comprises contacting the cell with the gRNA of any one of embodiments 330 to 578.

703. The method of embodiment 700, which comprises contacting the cell with the system of any one of embodiments 579 to 594.

704. The method of embodiment 703, which comprises electroporation of the cell prior to contacting the cell with the system.

705. The method of embodiment 703, which comprises lipid-mediated delivery of the system to the cell, optionally wherein the lipid-mediated delivery is cationic lipid-mediated delivery.

706. The method of embodiment 703, which comprises polymer-mediated delivery of the system to the cell.

707. The method of embodiment 703, which comprises delivery of the system to the cell by lipofection.

708. The method of embodiment 703, which comprises delivery of the system to the cell by nucleofection.

709. The method of embodiment 700, which comprises contacting the cell with the nucleic acid of any one of embodiments 595 to 654.

710. The method of embodiment 700, which comprises contacting the cell with the plurality of nucleic acids of any one of embodiments 655 to 659.

711. The method of embodiment 700, which comprises contacting the cell with the particle of any one of embodiments 667 to 687.

712. The method of embodiment 700, which comprises contacting the cell with the pharmaceutical composition of embodiment 688.

713. The method of any one of embodiments 700 to 712, further comprising contacting the cell with a DNA mismatch repair (MMR) inhibitor or nucleic acid encoding the MMR inhibitor, optionally wherein the MMR inhibitor comprises an amino acid sequence having at least 50%, at least 60%, at least 70%, at least 80%, at least 90%, at least 95%, at least 97%, at least 98%, at least 99%, or 100% sequence identity to SEQ ID NO:258.

714. The method of any one of embodiments 700 to 713, wherein the contacting alters a CCR5, EMX1, Fas, FANCF, HBB, ZSCAN2, Chr6, ADAMTSL1, B2M, CXCR4, PD1, DNMT1, Match8, TRAC, TRBC, VEGFAsite2, VEGFAsite3, CACNA, HEKsite3, HEKsite4, Chr8, BCR, ATM, HBG1, HPRT, IL2RG, NF1, USH2A, RHO, BcLenh, or CTFR genomic sequence 715. The method of any one of embodiments 700 to 713, wherein the contacting alters a RHO genomic sequence.

716. The method of any one of embodiments 700 to 713, wherein the contacting alters a TRAC genomic sequence.

717. The method of any one of embodiments 700 to 713, wherein the contacting alters a B2M genomic sequence.

718. The method of any one of embodiments 700 to 713, wherein the contacting alters a PD1 genomic sequence.

719. The method of any one of embodiments 700 to 713, wherein the contacting alters a LAG3 genomic sequence.

720. The method of any one of embodiments 700 to 713, wherein the contacting alters a AAVS1 genomic sequence. 721. The method of any one of embodiments 700 to 713, wherein the contacting alters an EMX1 genomic sequence.

722. The method of any one of embodiments 700 to 713, wherein the contacting alters a BCLA11A genomic sequence.

723. The method of any one of embodiments 700 to 713, wherein the contacting alters a PCSK9 genomic sequence.

724. The method of any one of embodiments 700 to 713, wherein the contacting alters a VEGFA genomic sequence.

725. The method of any one of embodiments 700 to 713, wherein the contacting alters a Match6 genomic sequence.

726. The method of any one of embodiments 700 to 725, wherein the cell is a human cell.

727. The method of any one of embodiments 700 to 726, wherein the cell is a hematopoietic progenitor cell.

728. The method of any one of embodiments 700 to 727, wherein the cell is a stem cell.

729. The method of embodiment 728, wherein the stem cell is a hematopoietic stem cell (HSC), a pluripotent stem cell, or an induced pluripotent stem cell (iPS).

730. The method of embodiment 729, wherein the stem cell is an embryonic stem cell.

731. The method of any one of embodiments 700 to 725, wherein the cell is a retinal cell. 732. The method of any one of embodiments 700 to 725, wherein the cell is a photoreceptor cell.

733. The method of any one of embodiments 700 to 725, wherein the cell is a T cell.

734. The method of any one of embodiments 700 to 733, wherein the contacting is in vitro.

735. The method of embodiment 731, further comprising transplanting the cell to a subject.

736. The method of any one of embodiments 700 to 730, wherein the contacting is in vivo in a subject.

737. A cell or population of cells produced by the method of any one of embodiments 700 to 734.

738. A Type V Cas protein according to any one of embodiments 1 to 329, the gRNA according to any one of embodiments 330 to 578, or the system of any one of embodiments 579 to 594 for use in a nucleic acid detection assay.

739. A method of detecting a target nucleic acid, comprising (a) combining a test sample with the Type V Cas protein of any one of embodiments 1 to 329, a gRNA comprising a spacer which is partially or fully complementary to a nucleotide sequence present in the target nucleic acid, and a reporter nucleic acid, and (b) detecting cleavage of the reporter nucleic acid, if any, whereby cleavage of the reporter nucleic acid indicates that the target nucleic acid is present in the test sample.

740. The method of embodiment 739, wherein the reporter nucleic acid comprises a quenched fluorescent reporter moiety.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1-30. (canceled)

31. A fusion protein comprising:
(a) a Type V Cas amino acid sequence comprising an amino acid sequence that is at least 90% identical to the full length of SEQ ID NO:43, SEQ ID NO:44, SEQ ID NO: 1, SEQ ID NO:2, SEQ ID NO:7, SEQ ID NO:8, SEQ ID NO: 13, SEQ ID NO: 14, SEQ ID NO: 19, SEQ ID NO:20, SEQ ID NO:25, SEQ ID NO:26, SEQ ID NO: 31, SEQ ID NO: 32, SEQ ID NO: 37, SEQ ID NO:38, SEQ ID NO:49, SEQ ID NO: 50, SEQ ID NO:55, SEQ ID NO:56, SEQ ID NO:61, SEQ ID NO:62, SEQ ID NO: 67, SEQ ID NO: 68, SEQ ID NO: 73, SEQ ID NO:74, SEQ ID NO:79, SEQ ID NO: 80, SEQ ID NO:85, SEQ ID NO: 86, SEQ ID NO:91, SEQ ID NO:92, SEQ ID NO: 97, SEQ ID NO:98, SEQ ID NO: 103, SEQ ID NO: 104, SEQ ID NO: 109, SEQ ID NO: 110, SEQ ID NO: 115, or SEQ ID NO:116; and
(b) one or more nuclear localization signals.

32. The fusion protein of claim 31, wherein the Type V Cas amino acid sequence comprises an amino acid sequence that is at least 95% identical to the full length of SEQ ID NO:43.

33. The fusion protein of claim 31, wherein the Type V Cas amino acid sequence comprises an amino acid sequence that is identical to SEQ ID NO:43.

34. The fusion protein of claim 31, wherein the Type V Cas amino acid sequence comprises an amino acid sequence that is identical to SEQ ID NO:44.

35. The fusion protein of claim 31, which comprises a C-terminal nuclear localization signal.

36. The fusion protein of claim 31, which comprises an N-terminal nuclear localization signal.

37. The fusion protein of claim 31, which comprises a nuclear localization signal comprising the amino acid sequence KRTADGSEFESPKKKRKV (SEQ ID NO:122), PKKKRKV (SEQ ID NO:123), PKKKRRV (SEQ ID NO:124), KRPAATKKAGQAKKKK (SEQ ID NO: 125), YGRKKRRQRRR (SEQ ID NO: 126), RKKRRQRRR (SEQ ID NO: 127), PAAKRVKLD (SEQ ID NO:128), RQRRNELKRSP (SEQ ID NO:129), VSRKRPRP (SEQ ID NO:130), PPKKARED (SEQ ID NO:131), PQPKKKPL (SEQ ID NO:132), SALIKKKKKMAP (SEQ ID NO: 133), PKQKKRK (SEQ ID NO: 134), RKLKKKIKKL (SEQ ID NO: 135), REKKKFLKRR (SEQ ID NO: 136), KRKGDEVDGVDEVAKKKSKK (SEQ ID NO:137), RKCLQAGMNLEARKTKK (SEQ ID NO: 138), NQSSNFGPMKGGNFGGRSSGPYGGGGQYFAKPRNQGGY (SEQ ID NO: 139), RMRIZFKNKGKDTAELRRRRVEVSVELRKAKKDEQILKRRNV (SEQ ID NO: 140), or SSDDEATADSQHAAPPKKKRKV (SEQ ID NO:178).

38. The fusion protein of claim 31, which comprises a nuclear localization signal comprising the amino acid sequence GRSSDDEATADSQHAAPPKKKRKV (SEQ ID NO: 180).

39. The fusion protein of claim 31, wherein the fusion protein comprises a Type V Cas amino acid sequence that is identical to SEQ ID NO:44 and a C-terminal nuclear localization signal comprising the amino acid sequence GRSSDDEATADSQHAAPPKKKRKV (SEQ ID NO:180).

40. A system comprising the fusion protein of claim 31 and a guide RNA (gRNA) comprising a spacer positioned 3′ to a crRNA scaffold and capable of forming a complex with the fusion protein and directing the fusion protein to a target DNA.

41. The system of claim 40, wherein the nucleotide sequence of the spacer is complementary to a target mammalian genomic sequence that is downstream of a NTTV, VTTV, NCTV, or TTTT protospacer adjacent motif (PAM) sequence.

42. The system of claim 40, wherein the crRNA scaffold comprises a nucleotide sequence that is at least 90% identical to SEQ ID NO: 151 or SEQ ID NO:211.

43. The system of claim 42, wherein the crRNA scaffold comprises a nucleotide sequence that is identical to SEQ ID NO: 151 or SEQ ID NO:211.

44. The system of claim 40, which is a ribonucleoprotein (RNP) comprising the fusion protein complexed to the gRNA.

45. A nucleic acid encoding the fusion protein of claim 31.

46. The nucleic acid of claim 45, wherein the nucleotide sequence encoding the fusion protein is codon optimized for expression in human cells.

47. The nucleic acid of claim 45, which is an adeno-associated virus (AAV) genome.

48. An adeno-associated virus (AAV) particle comprising the nucleic acid of claim 47.

49. An ex vivo human cell comprising the fusion protein of claim 31.

50. The ex vivo human cell of claim 49, which is a hematopoietic stem cell (HSC), pluripotent stem cell or an induced pluripotent stem cell (iPS).

51. An ex vivo human cell comprising the system of claim 40.

52. The ex vivo human cell of claim 51, which is a hematopoietic stem cell (HSC), pluripotent stem cell or an induced pluripotent stem cell (iPS).

53. A method for altering a cell comprising contacting the cell with the system of claim 40.

54. A guide RNA (gRNA) comprising a spacer and a crRNA scaffold, wherein:
(a) the spacer is positioned 3′ to the crRNA scaffold; and
(b) the nucleotide sequence of the crRNA scaffold comprises a nucleotide sequence that is at least 90% identical to the nucleotide sequence of any one of SEQ ID NOs: 144-163 or at least 90% identical to the nucleotide sequence of any one of SEQ ID NOs: 211-213.


SEQUENCE LISTING (412 sequences)

SEQ ID NO: 1 - protein, 1291 aa, unidentified
VSEKENTPTFNSLTNLYSVSKTLRFELRPQYSTLDHIKDDQIVDKGEELKNHYKTFKKILDQVFSRIINDSLDKTYLDQKYISTYQDLVFKHRDRLTDKDRAELKALKETLKKQIDKSLDHKDKKAIFSDPVNFLIDNESDFADLIGDNRPSIEAFNRQKGYLSGYLQNRANIFDHTTNETSVAFRIVEENLAIFLNNRLTLQHFFEKVADKDGLLKFLQETLSQLGFKLKLEDLLSLDYFNRTLSQPGIDQYNLLISGKALEDGKKMQGINEVLNQYLQQHQEEKLHKIKLKQLYKQILSESKTESFTLDFVEDNKGLAAMLLQFIDFVNKLIEEKMLLLDMIQGLKDSSVSSEFLSRLYLERKNIKRLSNFIYKDYGYIEQSLEENFLSTIEGKITKKALEEHRKQDAFTIHEILVALQKQQYEKDGALESADHLLLPGVVDFLYQNLDCKHSTLLEKVGSEKQPLLDLFNEKQLLEGQDAESHASKYSDRPFNDHEIKVVKTALDFYKNLQSNFAIFQIPDENLKLDSEFYSEFDEFYQGLKNIIPVYNKSRNFLTKKPFSTEKTKLIFNNPQLLDGWSKSKESDCLGTIFIKDGKYYVGIINSATNAKNTLFEPNNFANFDQKQYFEKMNLFFLSDLKRDFPKKYFSEKWHNQHPVPADLREKYDYYRIDEHKDERKNDLKYHHQLIAYYQDCLKKDTEWQIYQFKYKAPEEYSDVNEFLSELTPNTYKMEFNKIPAEYIKKLVDDGKLYFFQIYSKDFSEFAKGKPNLHTLYLKAVFDQKNAEEFNYNYKISGSAEIFYRPASIETRVTHPKNQPIKNKNKNNPKAESVFQYDLCKDRRYMSDKFFLHLPIELNRIPLLANDSSVNSMVNQVVSSRNQNYFLGIDRGERHLIYLVLIDQNGRIIKQQTLNQITSSYQEKANNQTVEVITDYHDLLNDKEKLRKKNLQEWQSVENIKELKAGYLSNVVNEIGKIIVEYQPVIMLENLNTGFKNSRIKIEKQVYQKFEKALIDKFNYFMRKDLDSSAIGGLYHALQLTKEYSKQYNGKQNGIIYYIPASYTSNIDPTTGFISAFIQTRYENVEKTKSLIEKFNDITYDAEESLFCFSADYKKFSPEAKLWQQTIWQIYTNGDRIYTFKNKEEWQSKNYILVEEFKDLFAKYHIDYCRDLKAQILSQTDASFFKQFLFLLRLTLQMRNSRTTELNGTDADTKKRENDYIISPVKNQYGKFYDSRKDYVDWPENADANGAYNIARKGLIMLKHLKEGLPEKRICDISTEEWVQFVEELNK

SEQ ID NO: 2 - protein, 1292 aa, unidentified
MVSEKENTPTFNSLTNLYSVSKTLRFELRPQYSTLDHIKDDQIVDKGEELKNHYKTFKKILDQVFSRIINDSLDKTYLDQKYISTYQDLVFKHRDRLTDKDRAELKALKETLKKQIDKSLDHKDKKAIFSDPVNFLIDNESDFADLIGDNRPSIEAFNRQKGYLSGYLQNRANIFDHTTNETSVAFRIVEENLAIFLNNRLTLQHFFEKVADKDGLLKFLQETLSQLGFKLKLEDLLSLDYFNRTLSQPGIDQYNLLISGKALEDGKKMQGINEVLNQYLQQHQEEKLHKIKLKQLYKQILSESKTESFTLDFVEDNKGLAAMLLQFIDFVNKLIEEKMLLLDMIQGLKDSSVSSEFLSRLYLERKNIKRLSNFIYKDYGYIEQSLEENFLSTIEGKITKKALEEHRKQDAFTIHEILVALQKQQYEKDGALESADHLLLPGVVDFLYQNLDCKHSTLLEKVGSEKQPLLDLFNEKQLLEGQDAESHASKYSDRPFNDHEIKVVKTALDFYKNLQSNFAIFQIPDENLKLDSEFYSEFDEFYQGLKNIIPVYNKSRNFLTKKPFSTEKTKLIFNNPQLLDGWSKSKESDCLGTIFIKDGKYYVGIINSATNAKNTLFEPNNFANFDQKQYFEKMNLFFLSDLKRDFPKKYFSEKWHNQHPVPADLREKYDYYRIDEHKDERKNDLKYHHQLIAYYQDCLKKDTEWQIYQFKYKAPEEYSDVNEFLSELTPNTYKMEFNKIPAEYIKKLVDDGKLYFFQIYSKDFSEFAKGKPNLHTLYLKAVFDQKNAEEFNYNYKISGSAEIFYRPASIETRVTHPKNQPIKNKNKNNPKAESVFQYDLCKDRRYMSDKFFLHLPIELNRIPLLANDSSVNSMVNQVVSSRNQNYFLGIDRGERHLIYLVLIDQNGRIIKQQTLNQITSSYQEKANNQTVEVITDYHDLLNDKEKLRKKNLQEWQSVENIKELKAGYLSNVVNEIGKIIVEYQPVIMLENLNTGFKNSRIKIEKQVYQKFEKALIDKFNYFMRKDLDSSAIGGLYHALQLTKEYSKQYNGKQNGIIYYIPASYTSNIDPTTGFISAFIQTRYENVEKTKSLIEKFNDITYDAEESLFCFSADYKKFSPEAKLWQQTIWQIYTNGDRIYTFKNKEEWQSKNYILVEEFKDLFAKYHIDYCRDLKAQILSQTDASFFKQFLFLLRLTLQMRNSRTTELNGTDADTKKRENDYIISPVKNQYGKFYDSRKDYVDWPENADANGAYNIARKGLIMLKHLKEGLPEKRICDISTEEWVQFVEELNK

SEQ ID NO: 3 - protein, 1329 aa, synthetic construct
MGVSEKENTPTFNSLTNLYSVSKTLRFELRPQYSTLDHIKDDQIVDKGEELKNHYKTFKKILDQVFSRIINDSLDKTYLDQKYISTYQDLVFKHRDRLTDKDRAELKALKETLKKQIDKSLDHKDKKAIFSDPVNFLIDNESDFADLIGDNRPSIEAFNRQKGYLSGYLQNRANIFDHTTNETSVAFRIVEENLAIFLNNRLTLQHFFEKVADKDGLLKFLQETLSQLGFKLKLEDLLSLDYFNRTLSQPGIDQYNLLISGKALEDGKKMQGINEVLNQYLQQHQEEKLHKIKLKQLYKQILSESKTESFTLDFVEDNKGLAAMLLQFIDFVNKLIEEKMLLLDMIQGLKDSSVSSEFLSRLYLERKNIKRLSNFIYKDYGYIEQSLEENFLSTIEGKITKKALEEHRKQDAFTIHEILVALQKQQYEKDGALESADHLLLPGVVDFLYQNLDCKHSTLLEKVGSEKQPLLDLFNEKQLLEGQDAESHASKYSDRPFNDHEIKVVKTALDFYKNLQSNFAIFQIPDENLKLDSEFYSEFDEFYQGLKNIIPVYNKSRNFLTKKPFSTEKTKLIFNNPQLLDGWSKSKESDCLGTIFIKDGKYYVGIINSATNAKNTLFEPNNFANFDQKQYFEKMNLFFLSDLKRDFPKKYFSEKWHNQHPVPADLREKYDYYRIDEHKDERKNDLKYHHQLIAYYQDCLKKDTEWQIYQFKYKAPEEYSDVNEFLSELTPNTYKMEFNKIPAEYIKKLVDDGKLYFFQIYSKDFSEFAKGKPNLHTLYLKAVFDQKNAEEFNYNYKISGSAEIFYRPASIETRVTHPKNQPIKNKNKNNPKAESVFQYDLCKDRRYMSDKFFLHLPIELNRIPLLANDSSVNSMVNQVVSSRNQNYFLGIDRGERHLIYLVLIDQNGRIIKQQTLNQITSSYQEKANNQTVEVITDYHDLLNDKEKLRKKNLQEWQSVENIKELKAGYLSNVVNEIGKIIVEYQPVIMLENLNTGFKNSRIKIEKQVYQKFEKALIDKFNYFMRKDLDSSAIGGLYHALQLTKEYSKQYNGKQNGIIYYIPASYTSNIDPTTGFISAFIQTRYENVEKTKSLIEKFNDITYDAEESLFCFSADYKKFSPEAKLWQQTIWQIYTNGDRIYTFKNKEEWQSKNYILVEEFKDLFAKYHIDYCRDLKAQILSQTDASFFKQFLFLLRLTLQMRNSRTTELNGTDADTKKRENDYIISPVKNQYGKFYDSRKDYVDWPENADANGAYNIARKGLIMLKHLKEGLPEKRICDISTEEWVQFVEELNKSRKRTADGSEFESPKKKRKVGSGKPIPNPLLGLDST

SEQ ID NO: 4 - DNA, 3879 bp, unidentified
  source 1..3879: mol_type = unassigned DNA
atggtgtccgaaaaagaaaatacaccaacttttaatagtctaaccaatctctatagtgtttcaaagactcttagatttgaacttaggccacaatattcaactctagatcacattaaagatgaccaaattgttgacaaaggtgaagaactaaaaaaccactacaaaactttcaagaaaattcttgatcaggtcttttcaaggatcatcaacgatagcctagataaaacctatcttgatcaaaaatatatttccacctaccaagatcttgtattcaagcatcgagaccgactaacagacaaagaccgtgcagaactaaaggccttaaaagaaacactcaaaaagcagatcgacaaaagcctcgatcataaagataaaaaagctatcttcagtgatcccgtaaattttctcatcgacaatgaatcggattttgctgacttaattggtgataatcgtcctagtattgaagctttcaaccgtcaaaaaggttatctttccggatatctccaaaatcgcgcaaatatcttcgatcacaccacaaatgaaacttcagtcgcgtttcgtattgtcgaggaaaacctcgctatctttttaaataatcgcctcacattacagcattttttcgagaaagttgcagataaagatgggctattaaaatttttacaagagacactttctcagttaggttttaagttgaaactcgaagaccttctttcccttgattattttaatcgtaccctatctcaacccggcatcgatcagtataacctcctaatctctggcaaggcgctagaagatggaaagaaaatgcagggaattaatgaggtcctcaatcaatatctccaacaacatcaagaagagaagctacataaaatcaaactcaagcaactctataagcagatcctctcagagtcaaaaactgaatcatttacccttgattttgtggaagataataaagggcttgctgccatgctcctacagtttatcgattttgtaaacaagctgattgaagagaaaatgcttctccttgatatgattcaggggctaaaagatagctcagtttcatcagaatttctttcacgactctatcttgaacgcaaaaacatcaagcgtctttcgaattttatctataaagattatggctatattgagcaatccttggaagagaactttctctcgacaattgaaggcaagattaccaagaaggcactcgaggaacatcgcaaacaggatgctttcacaatccatgaaatcttagttgccctacaaaagcaacaatatgaaaaggatggagctctagagtccgcagatcatcttttacttcctggtgttgttgacttcctctaccagaatttggattgcaaacactccactctacttgaaaaagtcgggtcagaaaaacagccactactcgacctcttcaacgaaaaacaattattggaaggtcaagacgcagaatctcatgcttccaaatattctgatcgtccattcaacgaccacgaaataaaggttgttaaaactgctttggatttttataaaaatctacagagtaattttgcgatctttcaaatcccggatgaaaaccttaaactagattccgaattttattccgagtttgatgaattttatcaaggtctcaagaatattattccagtctataacaagtccagaaatttcctcactaaaaaaccattctcaaccgaaaagaccaagctcatttttaacaacccgcaactacttgacggatggagtaaatcaaaagagtcagattgtttaggcacgatttttattaaagacggcaaatattatgttggcattattaatagtgctacgaatgctaaaaatactttatttgagcctaacaattttgcaaacttcgaccaaaaacaatattttgaaaagatgaaccttttcttcctttcggacttgaagcgagattttcctaagaaatatttttctgaaaagtggcataatcaacacccagttccagccgatcttcgtgaaaagtatgattattatcgaatcgacgaacataaggatgagcgcaaaaatgatctaaaatatcatcatcaacttatcgcctattatcaagactgtcttaaaaaagacacggaatggcagatttatcaattcaaatataaggcccctgaagaatattcagatgtcaatgaattcttatccgagcttactccaaatacctacaaaatggagttcaataaaatcccagctgaatatatcaaaaagcttgttgatgatggaaaattatatttcttccaaatttattccaaagatttttctgagtttgcaaaaggtaaaccaaatctccatactctctatctaaaagcggtctttgatcagaaaaatgcggaagagttcaactataattataaaatttctggtagtgccgaaatcttctatcgtccagccagcattgaaactcgtgtcactcatccaaaaaatcaaccaatcaagaataagaataaaaataatccaaaggctgaatctgtcttccagtatgatctttgtaaagatcgtcgctatatgtcagataaattctttttgcatcttccgatcgaattaaatcgtattccgttactcgctaacgactcctcggtaaatagtatggtcaatcaagtcgttagttctcgtaatcagaattatttccttggtattgaccgtggcgagaggcatctaatttatctagtcctgatcgatcaaaacggtagaatcattaaacagcaaaccttaaatcagatcactagttcataccaagaaaaagccaataaccaaacggttgaagttattacggattatcatgatctcttgaatgacaaagaaaaactgcgaaagaagaatctccaagagtggcaatccgtcgaaaatatcaaggagttaaaggctgggtacctaagtaatgtggtgaatgaaatcggtaagattatcgttgaatatcagccagttattatgctggaaaatcttaatactggatttaaaaactcacgaattaaaattgagaaacaggtgtaccagaaatttgagaaggcgctcattgataagtttaactactttatgagaaaagatctcgactcttcagctattggtggtctctatcacgctttgcagttgactaaggaatactctaagcagtacaacggcaagcagaatggtatcatctactatattcctgcaagctacactagtaatattgatccaactactggtttcatctcggcctttatacagactagatacgaaaacgtcgagaaaacaaaatccttaatcgaaaagtttaatgatatcacttatgatgcagaagaatctctcttctgcttctccgcagattacaagaaatttagtccagaggccaagctttggcagcagacgatttggcagatttatactaatggcgatcgtatttatacatttaagaacaaagaagagtggcagagcaaaaactacatcctcgttgaggagttcaaagatctctttgctaaatatcacatcgattattgcagggaccttaaggcgcagattctgtcacaaactgacgcgagcttcttcaagcagttcctcttcttgttgcgactaaccttgcagatgcgaaatagtcgcactaccgaattaaatggaactgatgctgatactaaaaaacgtgagaatgattatattatttctccagttaagaatcagtatggcaagttctatgattcccgcaaggattatgtggactggccagaaaatgcagatgcaaatggcgcatacaatattgccagaaaaggtctcatcatgctaaaacacctaaaagaaggtcttcccgaaaaacgtatctgtgatatatcgactgaagaatgggtacagtttgtcgaagaactaaataaatag

SEQ ID NO: 5 - DNA, 3873 bp, synthetic construct
  source 1..3873: mol_type = other DNA
gtgtctgaaaaggaaaacacccctaccttcaactctctgaccaacctgtacagcgtttctaaaaccctgcggttcgagctgcggcctcagtacagcaccctggaccacatcaaggacgatcagatcgtggacaagggagaggagctaaagaaccactacaagacattcaaaaaaatcctggaccaggtgttctctcggatcatcaacgactctctggataaaacttacctggatcagaagtacatctccacctaccaggatctggtgttcaagcacagagatagactgacagataaggacagagccgaactgaaggccctgaaggagacactgaagaagcagatcgacaaaagcctggatcacaaagacaagaaggctatcttctccgaccctgtgaacttcctgatcgacaatgagagcgacttcgccgacctgattggagacaaccggcccagcatcgaggcctttaaccgccagaagggatatctgtccggctacctgcagaatagagccaacatcttcgatcatacaaccaacgaaaccagcgttgctttcagaatcgtggaagagaacctcgccatcttcctcaacaaccgcctgaccctgcagcatttcttcgagaaagtggccgacaaagacggactgctgaagttcctgcaggagacactgagccagctgggcttcaagctgaagctggaggatctgctgagcctggattactttaaccggacactgagccagcctggcatcgaccaatacaacctgctgatcagcggaaaggccctggaagatggcaagaagatgcagggcatcaatgaagtgctgaaccagtacctgcagcagcaccaggaggaaaagctgcacaaaatcaagctgaagcagctgtataagcaaatcctgagcgaaagcaagacagagagcttcacgctggacttcgtggaggacaacaagggcctggccgccatgctgctgcagtttatcgatttcgtgaacaagttaatagaagagaagatgctgctgctggatatgatccagggactgaaagacagcagtgtgtccagcgagttcttgagccggctttacctggaaagaaagaacatcaagcggctgagcaacttcatctacaaggactatggctatatcgagcagtccctggaagaaaacttcctgagcaccatcgagggcaagatcactaagaaggccctggaagagcatagaaaacaggacgcctttaccattcacgagatcctggtcgcactgcagaaacaacagtacgaaaaggacggcgccctagagagcgccgaccacctgctgcttccaggcgtggtggatttcctctaccaaaacctggactgtaagcacagcacgctgctggaaaaggtgggcagcgagaagcagcccctgctggatcttttcaacgaaaagcagctgcttgagggccaggacgccgagtcccacgcctctaagtacagcgatcggcctttcaacgaccacgagatcaaggtggtgaaaaccgccctggacttctacaagaacctgcaatctaactttgctatcttccagatccccgacgaaaacctgaagctggatagcgagttttacagcgagtttgatgagttctaccagggcctgaaaaatattattcctgtgtacaacaaaagccggaacttcctgacaaaaaagccgttcagcaccgaaaagaccaaactgatcttcaacaacccccagctgctcgatggctggagcaagagcaaggaaagcgactgtctggggaccatcttcatcaaagacggcaagtactatgtgggaatcatcaacagcgccaccaacgctaagaatacactgttcgagcctaacaacttcgccaatttcgaccaaaaacaatacttcgagaagatgaacctgttcttcctgagcgatctgaagcgagacttccccaagaagtatttctccgagaagtggcacaaccagcaccccgtgcccgctgaccttagagaaaagtacgactactaccggatcgacgagcataaggatgagagaaagaatgacctgaaataccaccaccagttaatcgcctactaccaagactgcctgaaaaaggatacagagtggcagatctaccagttcaagtacaaggcccctgaggagtacagcgacgtgaacgagttcctgagtgaactgacccctaatacctacaagatggagttcaacaagattcctgccgagtacattaagaagctggtggatgacggcaagctgtacttttttcagatatactccaaagactttagcgaatttgccaagggcaagccaaacctgcacaccctctacctgaaggccgtgttcgaccagaagaacgccgaggagttcaactacaactataaaatatctggatctgctgaaatcttttacagacctgcttctatcgagacaagagtgacccaccctaagaatcagcctatcaagaacaagaacaagaacaatcctaaggctgaaagcgtgttccagtacgacctgtgcaaggaccggcggtacatgtccgacaagttcttcctgcaccttcccatcgaacttaacagaatccctctgctggctaacgattcctccgtgaatagcatggtcaaccaggtggtgagcagcagaaaccagaactacttcctgggcatcgatagaggcgagagacacctgatctacctggtgctgatcgaccagaacggtagaatcatcaagcaacagaccctgaatcagattacaagcagctaccaagaaaaggccaacaaccagacagtggaggtgatcacagactaccacgacctgctgaacgacaaggaaaagctcagaaagaagaatcttcaggagtggcagtccgtggagaatatcaaagagctgaaggccggctacctgagcaacgtggtcaacgagatcggcaagatcatcgtggagtaccagcctgtgatcatgctggaaaacctcaacaccggatttaaaaactcaagaatcaagattgagaagcaggtgtaccagaagttcgagaaggccttaatcgataagttcaattacttcatgcggaaggatctggactctagcgccatcggcggcctgtaccacgccctgcagctgaccaaagagtatagcaagcagtacaacggcaagcagaacggcatcatctactacatcccagcttcttacacctctaatatcgaccccaccaccggctttattagcgccttcatccagaccagatacgagaacgtggaaaagaccaagtctctgatcgagaaatttaatgacatcacctacgacgccgaagagtcgctgttctgcttcagcgccgattacaagaaattttcacctgaagctaagctgtggcagcaaaccatctggcagatctataccaacggcgacagaatctacaccttcaagaacaaggaagagtggcaaagcaagaactacattctggtggaggagtttaaggacctgttcgccaaataccacatcgactattgcagggacctgaaagcccagatcctgagccagaccgacgcatcttttttcaagcagtttctcttcctgctgagactgacactgcaaatgagaaatagtcgtaccacagagctgaacggcaccgacgccgacaccaagaaaagagagaatgactacatcatctctccagtgaaaaatcagtacggcaaattctatgattcccgcaaggactacgtggactggcctgagaacgccgacgccaatggcgcctacaacatcgccagaaagggcctgatcatgctgaagcacctgaaggaaggactgcctgagaagaggatctgcgacatcagcacagaagaatgggttcagtttgtggaagaactgaacaag

SEQ ID NO: 6 - DNA, 3990 bp, synthetic construct
  source 1..3990: mol_type = other DNA
atgggcgtgtctgaaaaggaaaacacccctaccttcaactctctgaccaacctgtacagcgtttctaaaaccctgcggttcgagctgcggcctcagtacagcaccctggaccacatcaaggacgatcagatcgtggacaagggagaggagctaaagaaccactacaagacattcaaaaaaatcctggaccaggtgttctctcggatcatcaacgactctctggataaaacttacctggatcagaagtacatctccacctaccaggatctggtgttcaagcacagagatagactgacagataaggacagagccgaactgaaggccctgaaggagacactgaagaagcagatcgacaaaagcctggatcacaaagacaagaaggctatcttctccgaccctgtgaacttcctgatcgacaatgagagcgacttcgccgacctgattggagacaaccggcccagcatcgaggcctttaaccgccagaagggatatctgtccggctacctgcagaatagagccaacatcttcgatcatacaaccaacgaaaccagcgttgctttcagaatcgtggaagagaacctcgccatcttcctcaacaaccgcctgaccctgcagcatttcttcgagaaagtggccgacaaagacggactgctgaagttcctgcaggagacactgagccagctgggcttcaagctgaagctggaggatctgctgagcctggattactttaaccggacactgagccagcctggcatcgaccaatacaacctgctgatcagcggaaaggccctggaagatggcaagaagatgcagggcatcaatgaagtgctgaaccagtacctgcagcagcaccaggaggaaaagctgcacaaaatcaagctgaagcagctgtataagcaaatcctgagcgaaagcaagacagagagcttcacgctggacttcgtggaggacaacaagggcctggccgccatgctgctgcagtttatcgatttcgtgaacaagttaatagaagagaagatgctgctgctggatatgatccagggactgaaagacagcagtgtgtccagcgagttcttgagccggctttacctggaaagaaagaacatcaagcggctgagcaacttcatctacaaggactatggctatatcgagcagtccctggaagaaaacttcctgagcaccatcgagggcaagatcactaagaaggccctggaagagcatagaaaacaggacgcctttaccattcacgagatcctggtcgcactgcagaaacaacagtacgaaaaggacggcgccctagagagcgccgaccacctgctgcttccaggcgtggtggatttcctctaccaaaacctggactgtaagcacagcacgctgctggaaaaggtgggcagcgagaagcagcccctgctggatcttttcaacgaaaagcagctgcttgagggccaggacgccgagtcccacgcctctaagtacagcgatcggcctttcaacgaccacgagatcaaggtggtgaaaaccgccctggacttctacaagaacctgcaatctaactttgctatcttccagatccccgacgaaaacctgaagctggatagcgagttttacagcgagtttgatgagttctaccagggcctgaaaaatattattcctgtgtacaacaaaagccggaacttcctgacaaaaaagccgttcagcaccgaaaagaccaaactgatcttcaacaacccccagctgctcgatggctggagcaagagcaaggaaagcgactgtctggggaccatcttcatcaaagacggcaagtactatgtgggaatcatcaacagcgccaccaacgctaagaatacactgttcgagcctaacaacttcgccaatttcgaccaaaaacaatacttcgagaagatgaacctgttcttcctgagcgatctgaagcgagacttccccaagaagtatttctccgagaagtggcacaaccagcaccccgtgcccgctgaccttagagaaaagtacgactactaccggatcgacgagcataaggatgagagaaagaatgacctgaaataccaccaccagttaatcgcctactaccaagactgcctgaaaaaggatacagagtggcagatctaccagttcaagtacaaggcccctgaggagtacagcgacgtgaacgagttcctgagtgaactgacccctaatacctacaagatggagttcaacaagattcctgccgagtacattaagaagctggtggatgacggcaagctgtacttttttcagatatactccaaagactttagcgaatttgccaagggcaagccaaacctgcacaccctctacctgaaggccgtgttcgaccagaagaacgccgaggagttcaactacaactataaaatatctggatctgctgaaatcttttacagacctgcttctatcgagacaagagtgacccaccctaagaatcagcctatcaagaacaagaacaagaacaatcctaaggctgaaagcgtgttccagtacgacctgtgcaaggaccggcggtacatgtccgacaagttcttcctgcaccttcccatcgaacttaacagaatccctctgctggctaacgattcctccgtgaatagcatggtcaaccaggtggtgagcagcagaaaccagaactacttcctgggcatcgatagaggcgagagacacctgatctacctggtgctgatcgaccagaacggtagaatcatcaagcaacagaccctgaatcagattacaagcagctaccaagaaaaggccaacaaccagacagtggaggtgatcacagactaccacgacctgctgaacgacaaggaaaagctcagaaagaagaatcttcaggagtggcagtccgtggagaatatcaaagagctgaaggccggctacctgagcaacgtggtcaacgagatcggcaagatcatcgtggagtaccagcctgtgatcatgctggaaaacctcaacaccggatttaaaaactcaagaatcaagattgagaagcaggtgtaccagaagttcgagaaggccttaatcgataagttcaattacttcatgcggaaggatctggactctagcgccatcggcggcctgtaccacgccctgcagctgaccaaagagtatagcaagcagtacaacggcaagcagaacggcatcatctactacatcccagcttcttacacctctaatatcgaccccaccaccggctttattagcgccttcatccagaccagatacgagaacgtggaaaagaccaagtctctgatcgagaaatttaatgacatcacctacgacgccgaagagtcgctgttctgcttcagcgccgattacaagaaattttcacctgaagctaagctgtggcagcaaaccatctggcagatctataccaacggcgacagaatctacaccttcaagaacaaggaagagtggcaaagcaagaactacattctggtggaggagtttaaggacctgttcgccaaataccacatcgactattgcagggacctgaaagcccagatcctgagccagaccgacgcatcttttttcaagcagtttctcttcctgctgagactgacactgcaaatgagaaatagtcgtaccacagagctgaacggcaccgacgccgacaccaagaaaagagagaatgactacatcatctctccagtgaaaaatcagtacggcaaattctatgattcccgcaaggactacgtggactggcctgagaacgccgacgccaatggcgcctacaacatcgccagaaagggcctgatcatgctgaagcacctgaaggaaggactgcctgagaagaggatctgcgacatcagcacagaagaatgggttcagtttgtggaagaactgaacaagtctagaaagcggacagcagacggctccgaatttgaaagccctaagaaaaagagaaaggtgggatccggcaaacctatccccaatcccctgctgggcctggacagcacctga

SEQ ID NO: 7 - protein, 1293 aa, unidentified
KSIYENFIGLESKNLTLRFALNPEAKTQENLKLYWDKLRDEERDRAYPIVKKILDKEYQQLISEGLKLLENQNVLDWTELAEYIRTSDLSKKKKEDKRLRKLIAQNLKAHPLVDKLKVKNAFGKNGYLETLPLGKEEKEAVKVFAGFGGFFNNYNKNRENYFSTEEKSTAIANRIVNENFSKHFSNVEIVTKIQKEVPELIQIVEAQFKGYDTIFTVNGYNTALSQAGIDTYNEMVAIWNKEANLYAQKAGKLPDGHPLKKKRNYLLSALFKQIGSEKEHLIQIDRFDGDEEVIEALTGVKKMLQEADVFEKLNMLVEDMENWDYSKIYLSAQSLSNVSVFLNNLYEDERENSWNYLDNVLREKWQIELQGKKKGTDLEEAIRKKKQSFYSIEELQEAVNAIEETDKCYNVSKWLLGAMKSERVIEEKKKDVEDFCTQWKNERNSLKETDITALKEYLEQWIFLARYCKSFYANGIEKKEKDEAFYHILEDVLYVLDEVIYFYNKVRNYVTKKPYSLEKMHLKFGHNELANGWSVNKEENYGTAILRRNGKYYLAITNSLNKKMSIPTQLESTGNNYEKMVLNVFPNVFRMIPKCTTGRNDVKSCFERKEPNEYFFIDTPEFVNPFKVTREEYELNKITYDGVKKWQSDYSKNTQDEKGYKEAVTKWIQFCMRFLQSYKSTAIYDYSTLQQPEKYETVDSFYHDVEKILYECHFEYVPANKIEQLEEEGRIFLFQIYNKDFSENRRPDSKKNLHTLYWEALFSEENRKAKVIQLNGKAEIFRREKSIEHPIVHKAGEVLVNKRTKDGEPIPDDIYKDLSNYFNGRNVTSEKEEYKECLDKVYTSTKKYDITKDKRFTETKYEFHVPITLNYQADGVKYLNQKILHVLRDNPDVNIIGLDRGERNLISYVVLNREGKIVNNQQGSFNIVGKMDYQKKLYQKEKNRDKERKTWKNIETIKDLKEGYISQVVHELTDMAIRNNAIIVMEDLNFGFKRGRTKVERQVYQKFELALLKKLHYLVTDKTEGEAMLKPGGVLQGYQLAREVKTLKEIGKQCGCVFYVPPGYTSKIDPTTGFVDVFNMSGVTNREKKKAFFEKFDNMFYDEKRDMFGFSFNYEKFTTYQSSYRNDWTVYSNGSKYVWNSLKRTDELIDVTKELKLLFEKYAIDYRNEALFEQIMSQDTDKNNADFWNKLFWYFRVLLRLRNSSDELDQIVSPVLNQNGEFFETPKKITEKSYLSDYPMDADTNGAYHIALKGLYLIQEKIADESVDLDNKLPKDFYKISNAEWFMFRQKEK

SEQ ID NO: 8 - protein, 1294 aa, unidentified
MKSIYENFIGLESKNLTLRFALNPEAKTQENLKLYWDKLRDEERDRAYPIVKKILDKEYQQLISEGLKLLENQNVLDWTELAEYIRTSDLSKKKKEDKRLRKLIAQNLKAHPLVDKLKVKNAFGKNGYLETLPLGKEEKEAVKVFAGFGGFFNNYNKNRENYFSTEEKSTAIANRIVNENFSKHFSNVEIVTKIQKEVPELIQIVEAQFKGYDTIFTVNGYNTALSQAGIDTYNEMVAIWNKEANLYAQKAGKLPDGHPLKKKRNYLLSALFKQIGSEKEHLIQIDRFDGDEEVIEALTGVKKMLQEADVFEKLNMLVEDMENWDYSKIYLSAQSLSNVSVFLNNLYEDERENSWNYLDNVLREKWQIELQGKKKGTDLEEAIRKKKQSFYSIEELQEAVNAIEETDKCYNVSKWLLGAMKSERVIEEKKKDVEDFCTQWKNERNSLKETDITALKEYLEQWIFLARYCKSFYANGIEKKEKDEAFYHILEDVLYVLDEVIYFYNKVRNYVTKKPYSLEKMHLKFGHNELANGWSVNKEENYGTAILRRNGKYYLAITNSLNKKMSIPTQLESTGNNYEKMVLNVFPNVFRMIPKCTTGRNDVKSCFERKEPNEYFFIDTPEFVNPFKVTREEYELNKITYDGVKKWQSDYSKNTQDEKGYKEAVTKWIQFCMRFLQSYKSTAIYDYSTLQQPEKYETVDSFYHDVEKILYECHFEYVPANKIEQLEEEGRIFLFQIYNKDFSENRRPDSKKNLHTLYWEALFSEENRKAKVIQLNGKAEIFRREKSIEHPIVHKAGEVLVNKRTKDGEPIPDDIYKDLSNYFNGRNVTSEKEEYKECLDKVYTSTKKYDITKDKRFTETKYEFHVPITLNYQADGVKYLNQKILHVLRDNPDVNIIGLDRGERNLISYVVLNREGKIVNNQQGSFNIVGKMDYQKKLYQKEKNRDKERKTWKNIETIKDLKEGYISQVVHELTDMAIRNNAIIVMEDLNFGFKRGRTKVERQVYQKFELALLKKLHYLVTDKTEGEAMLKPGGVLQGYQLAREVKTLKEIGKQCGCVFYVPPGYTSKIDPTTGFVDVFNMSGVTNREKKKAFFEKFDNMFYDEKRDMFGFSFNYEKFTTYQSSYRNDWTVYSNGSKYVWNSLKRTDELIDVTKELKLLFEKYAIDYRNEALFEQIMSQDTDKNNADFWNKLFWYFRVLLRLRNSSDELDQIVSPVLNQNGEFFETPKKITEKSYLSDYPMDADTNGAYHIALKGLYLIQEKIADESVDLDNKLPKDFYKISNAEWFMFRQKEK

SEQ ID NO: 9 - protein, 1331 aa, synthetic construct
MGKSIYENFIGLESKNLTLRFALNPEAKTQENLKLYWDKLRDEERDRAYPIVKKILDKEYQQLISEGLKLLENQNVLDWTELAEYIRTSDLSKKKKEDKRLRKLIAQNLKAHPLVDKLKVKNAFGKNGYLETLPLGKEEKEAVKVFAGFGGFFNNYNKNRENYFSTEEKSTAIANRIVNENFSKHFSNVEIVTKIQKEVPELIQIVEAQFKGYDTIFTVNGYNTALSQAGIDTYNEMVAIWNKEANLYAQKAGKLPDGHPLKKKRNYLLSALFKQIGSEKEHLIQIDRFDGDEEVIEALTGVKKMLQEADVFEKLNMLVEDMENWDYSKIYLSAQSLSNVSVFLNNLYEDERENSWNYLDNVLREKWQIELQGKKKGTDLEEAIRKKKQSFYSIEELQEAVNAIEETDKCYNVSKWLLGAMKSERVIEEKKKDVEDFCTQWKNERNSLKETDITALKEYLEQWIFLARYCKSFYANGIEKKEKDEAFYHILEDVLYVLDEVIYFYNKVRNYVTKKPYSLEKMHLKFGHNELANGWSVNKEENYGTAILRRNGKYYLAITNSLNKKMSIPTQLESTGNNYEKMVLNVFPNVFRMIPKCTTGRNDVKSCFERKEPNEYFFIDTPEFVNPFKVTREEYELNKITYDGVKKWQSDYSKNTQDEKGYKEAVTKWIQFCMRFLQSYKSTAIYDYSTLQQPEKYETVDSFYHDVEKILYECHFEYVPANKIEQLEEEGRIFLFQIYNKDFSENRRPDSKKNLHTLYWEALFSEENRKAKVIQLNGKAEIFRREKSIEHPIVHKAGEVLVNKRTKDGEPIPDDIYKDLSNYFNGRNVTSEKEEYKECLDKVYTSTKKYDITKDKRFTETKYEFHVPITLNYQADGVKYLNQKILHVLRDNPDVNIIGLDRGERNLISYVVLNREGKIVNNQQGSFNIVGKMDYQKKLYQKEKNRDKERKTWKNIETIKDLKEGYISQVVHELTDMAIRNNAIIVMEDLNFGFKRGRTKVERQVYQKFELALLKKLHYLVTDKTEGEAMLKPGGVLQGYQLAREVKTLKEIGKQCGCVFYVPPGYTSKIDPTTGFVDVFNMSGVTNREKKKAFFEKFDNMFYDEKRDMFGFSFNYEKFTTYQSSYRNDWTVYSNGSKYVWNSLKRTDELIDVTKELKLLFEKYAIDYRNEALFEQIMSQDTDKNNADFWNKLFWYFRVLLRLRNSSDELDQIVSPVLNQNGEFFETPKKITEKSYLSDYPMDADTNGAYHIALKGLYLIQEKIADESVDLDNKLPKDFYKISNAEWFMFRQKEKSRKRTADGSEFESPKKKRKVGSGKPIPNPLLGLDST

SEQ ID NO: 10 - DNA, 3885 bp, unidentified
  source 1..3885: mol_type = unassigned DNA
atgaaaagtatttatgaaaattttattggattggagtcaaaaaatttgacgctgcgctttgcgttgaatccagaagctaagacacaagaaaatttgaagttgtactgggacaaattgcgtgatgaggagagagatagggcgtatccaattgtaaaaaagatattggataaggaatatcagcagctgatttcggaaggactgaaattattagagaatcagaatgtgttggattggacagaattagcagagtatatacggacaagtgatttaagtaagaagaaaaaagaagataaacgcttaagaaaattaatagcacaaaatttaaaagcgcatccgttagttgacaaactgaaagtaaaaaatgcatttggtaaaaatggctatcttgaaactttaccgttgggaaaagaagagaaagaggcagtaaaagtttttgccggttttggcggctttttcaataactacaataaaaacagggaaaattatttttcaaccgaggaaaaaagcactgcaatcgcaaaccgaattgtaaatgaaaatttttcaaaacatttttcaaatgtagaaatagttaccaaaattcaaaaggaagtgccagaattaattcaaatcgtggaagcacaattcaagggatatgatactatctttacagtaaatggttataatacggcattgtcacaggcagggattgatacatataatgagatggttgcaatctggaataaagaagcaaatttgtatgcgcaaaaggcaggaaaacttccagatggacatccgttaaagaaaaagagaaattacttattgtcggcattgtttaaacagattgggagtgaaaaggagcatttgattcaaattgatagatttgatggagatgaagaggtgattgaggcattgacgggtgtgaaaaaaatgcttcaagaggcagatgtatttgaaaaattgaatatgcttgtggaggatatggagaattgggattatagtaaaatatatttgtcagcacagagtttatccaatgtttctgtgttcctaaataatttatatgaggatgaacgggagaactcatggaattatcttgataatgtcctaagagaaaaatggcaaatagaattacagggaaagaaaaaggggacagatctggaagaagcgattcggaagaaaaaacaaagtttctattcaatagaagaacttcaagaggcagtgaatgccatagaagaaacagataaatgttataatgtatctaaatggcttctaggagcaatgaaaagcgaaagggtaatagaagaaaaaaagaaggatgtggaagatttttgcacacagtggaaaaatgaaagaaactcgctgaaagagacagatataactgcactgaaagaatatctggagcaatggatttttttggcaagatattgcaaatctttttatgcaaatggaattgaaaaaaaagaaaaagatgaagcattttatcatattttagaagatgtgttgtatgttttggatgaagtaatatatttttataataaagttcgaaattatgtaacgaagaagccatattctcttgaaaaaatgcatttaaaatttggtcataatgaactggcaaatggatggtctgttaacaaagaagagaactatggtacggcaatattgaggcgaaatggcaaatactatttggcaattacaaattcattgaataaaaagatgagtattcccactcaattagaaagtacaggaaataattatgaaaagatggtattgaatgtattcccaaatgtatttcggatgataccaaaatgtactacaggaagaaatgatgtgaaaagttgttttgaaagaaaagagccaaatgagtatttctttattgatacaccggaatttgttaacccatttaaagttacgcgcgaggaatatgagttaaataagataacttatgatggtgttaaaaagtggcaatctgattattcaaaaaatacgcaggatgaaaaaggatacaaagaggcagtgacaaaatggattcagttttgtatgcgctttttacaatcttataagagtacagcaatatatgattattcaactttacagcaaccggagaaatatgagacggtggattctttttatcatgacgttgaaaaaatattatatgaatgtcattttgagtacgttccggctaataaaatagagcagttggaagaagaaggaagaatttttctgtttcagatttacaacaaagatttttcggaaaacagacgcccggacagcaaaaagaatttgcatacactttattgggaggcattgttttcagaagaaaatcggaaagcaaaagtgatacaattaaatggcaaagctgaaatatttcggagagaaaaaagcattgaacatccgattgttcataaagctggggaagtgttagtgaataaacgaacgaaagacggggaaccaataccagatgatatttataaagatttgagcaactattttaacggaagaaatgtaacatctgaaaaggaagagtataaggaatgtctggataaagtgtatacttcgaccaaaaaatatgatattacaaaggataaacgttttactgaaaccaaatatgaatttcatgttccgattaccttgaactatcaggcggacggtgttaaatatttgaatcagaaaatacttcatgtgctgagggataatccagatgtgaatattataggtctagatagaggcgagcgtaatctgatttcctacgtagtattgaaccgagaaggcaagattgttaacaatcagcaggggagtttcaatattgtgggtaagatggactatcagaagaaactgtatcaaaaagaaaagaatcgtgacaaagaacgaaaaacttggaaaaatatcgaaacaataaaggatttgaaggaaggatatatttcacaagtcgttcatgaattgaccgatatggcgattcgcaataatgcaattattgtgatggaagatctgaattttggatttaaaaggggacgcaccaaagtggaacggcaggtatatcagaagtttgagctggcgcttctgaagaaattgcattatctggttacggataaaacagaaggtgaggctatgcttaagcctggcggtgtccttcaaggttatcagcttgcaagagaagtaaaaaccctaaaagaaatcggaaagcaatgcggatgtgtattttatgttccaccgggatatacttctaaaatcgatccaacaaccggatttgttgatgtgtttaacatgtcaggtgttacgaatcgtgaaaagaaaaaagcattttttgaaaagttcgataatatgttctatgatgaaaagcgggatatgtttggattttcatttaactatgagaagtttacaacatatcaaagttcttatagaaatgattggactgtatattcgaatggaagcaaatatgtgtggaactctttaaaaaggacagacgagcttattgatgttacaaaagaattgaaactgctctttgaaaagtatgcaattgattacagaaacgaagcattgtttgaacaaatcatgtcccaagatacggataaaaacaatgctgacttttggaataaattgttctggtattttcgtgttttgctccgtctgagaaacagttcagatgaattagatcagattgtttcaccggtacttaatcaaaacggagaattttttgaaacaccgaaaaaaatcacggagaaaagttatttgtctgattatccgatggatgcggataccaatggtgcgtatcacatcgctttaaaagggttgtatctcatacaggaaaaaattgcagatgagagcgtagatttggataacaaattaccaaaagatttttacaagatctctaatgcagagtggtttatgtttaggcagaaggagaagtaa

SEQ ID NO: 11 - DNA, 3879 bp, synthetic construct
  source 1..3879: mol_type = other DNA
aagagcatctacgagaacttcatcggtcttgagagcaagaacctgacactgagattcgccctgaaccctgaggctaaaacccaggagaacctgaagctgtactgggacaaactgagggacgaagaaagagatagagcctaccctatcgtgaaaaaaatcctcgacaaggagtatcagcagctcatcagcgagggcctgaaactgctggaaaatcaaaacgtgctggactggaccgaactggccgagtacatcagaaccagcgatctgtctaagaagaagaaggaggacaagagactgcgcaagctgatcgcccagaacctgaaagcccaccccctggtcgacaagctgaaggtgaagaatgccttcggcaagaacggctacctggaaaccctgccattaggaaaggaagaaaaagaggccgtgaaggtgtttgccggattcggaggctttttcaacaactacaacaagaatcgggagaactacttcagtaccgaggagaagtccaccgccatcgccaacagaatcgtgaacgagaacttcagcaagcacttcagcaacgtggaaatcgttacaaagatccaaaaagaagtgccagagctgattcaaatcgtggaagctcagttcaagggttacgacaccatctttaccgtgaacggctacaacaccgccctgagccaggctggcatcgacacatacaacgaaatggtggccatctggaacaaggaggcaaacctgtacgctcaaaaagccggcaagctgccagacggccacccgctgaagaagaagcgtaactacctgctgagcgccctcttcaaacagatcggcagcgaaaaagaacacctgatccagatcgacagattcgacggcgacgaggaagtgatcgaagccctgactggcgtgaaaaagatgctgcaggaggccgacgtgttcgagaagctgaacatgctggtcgaggacatggaaaattgggattactccaagatctacctgtctgcccagagcctgagtaacgtgtccgtgttcctgaacaacctgtatgaagatgaacgggagaacagctggaactacctggataacgtgctgagagagaagtggcagattgaactgcagggcaaaaaaaagggaacagatctggaagaggccattagaaagaagaagcagagcttttactctatcgaggaacttcaggaggcagtgaacgccatcgaggaaaccgacaagtgctacaatgtgtctaaatggctgctgggagccatgaagagcgagagagtgatcgaggagaagaagaaagacgtggaggatttctgcacacagtggaagaacgagagaaacagcctcaaggaaaccgacatcaccgccctgaaggagtacctggagcagtggatcttcctggctaggtactgcaagagcttctacgccaatggcatcgaaaagaaagagaaggatgaggctttttaccacatcctggaggatgtgctgtacgtgctggacgaagtgatctacttctacaacaaggtgcggaactacgtgaccaaaaagccttacagtctggagaagatgcacctgaagttcggccacaacgagctggccaacggctggagcgtgaacaaggaagaaaattacggcaccgccatcctgagaagaaacggcaagtactacctggccatcaccaacagcctgaacaagaaaatgagcatccctacccagctggagagcacaggaaataattatgagaagatggtcctgaacgtttttcccaacgtgttccggatgatcccaaagtgcaccacaggcaggaacgacgtgaagtcatgcttcgagagaaaggaacccaacgagtacttcttcatcgacacccctgagttcgtgaacccctttaaggtcacacgggaggagtacgaactgaataagatcacctacgacggagttaagaagtggcagagcgactacagcaagaacacacaggacgaaaagggctataaggaagccgtgaccaagtggattcagttttgtatgcggttcctgcagtcttataagagcaccgccatatatgactacagcaccctgcagcaacctgaaaaatacgaaacagtggacagcttctatcatgatgtggaaaagatcctgtacgagtgccacttcgagtacgtgcccgctaacaagatcgagcagcttgaagaagagggaagaatcttcctgttccagatctacaacaaggatttttctgagaacagacggcctgatagcaagaaaaacctccacaccctgtactgggaggcgctgttctccgaagagaatagaaaggccaaggtgattcagctgaatggcaaggccgagatcttcagacgggagaaatcaatcgagcaccctatcgtgcataaggctggcgaggtgctggtgaacaagcggaccaaagatggcgaacctattcctgacgacatctacaaggacctgagcaactatttcaacggcagaaacgttacctctgagaaggaagagtacaaggagtgtctggacaaggtgtacaccagcaccaaaaagtacgatatcaccaaggacaaaagattcaccgagacaaagtacgagttccacgtgcctatcaccctgaactaccaggccgacggcgtgaagtacctgaatcagaagatcctgcacgtgctgcgggacaaccctgatgttaacatcatcggcctggatagaggcgaaagaaacctgatctcttatgttgtgctgaacagagagggcaagatcgtgaacaatcagcagggttctttcaacatcgtgggcaaaatggactaccagaaaaagctgtaccagaaggagaaaaaccgggataaagaacggaaaacgtggaaaaacatcgaaaccatcaaggacctgaaggagggctatatcagccaggtggtacacgagctgaccgatatggccatccggaataacgcgatcatcgtgatggaagatctgaatttcggattcaagcggggccggaccaaggtggaacggcaggtgtaccagaagtttgagctggccctgctgaagaagctgcactacctcgtgaccgacaagaccgagggagaagctatgctgaaacccggcggcgtgctgcaaggctaccagctggctagagaagtcaagaccctgaaagagatcggcaagcagtgcggctgtgtgttctacgtgccccctggctacacaagcaagatcgaccctacaaccggcttcgtcgacgtgttcaacatgtctggagttacaaaccgcgagaaaaagaaagcctttttcgaaaaatttgataacatgttctacgacgagaagagagacatgttcggcttcagcttcaattacgaaaagtttactacctaccagagcagctacagaaacgactggaccgtgtacagcaacggcagcaagtatgtgtggaactcccttaagagaacagacgagttaattgacgtgacaaaggagctcaagctgctgttcgagaagtacgccatcgattaccggaacgaagctctgtttgagcagatcatgagccaggatacagataagaacaacgccgacttctggaacaaactgttctggtacttccgggtgctgctgcggctgagaaatagcagcgacgaactggaccaaatcgtcagccctgtgctgaatcagaacggagagttcttcgaaacccctaagaaaatcacagagaagtcctacctgtctgattaccctatggacgccgatacaaacggcgcctaccacatcgccctgaagggcctgtacctgatccaggagaagatcgctgacgaatctgtggacctggacaacaagctgcctaaggacttctacaagatcagcaacgccgagtggttcatgtttagacagaaagaaaaa

SEQ ID NO: 12 - DNA, 3996 bp, synthetic construct
  source 1..3996: mol_type = other DNA
atgggcaagagcatctacgagaacttcatcggtcttgagagcaagaacctgacactgagattcgccctgaaccctgaggctaaaacccaggagaacctgaagctgtactgggacaaactgagggacgaagaaagagatagagcctaccctatcgtgaaaaaaatcctcgacaaggagtatcagcagctcatcagcgagggcctgaaactgctggaaaatcaaaacgtgctggactggaccgaactggccgagtacatcagaaccagcgatctgtctaagaagaagaaggaggacaagagactgcgcaagctgatcgcccagaacctgaaagcccaccccctggtcgacaagctgaaggtgaagaatgccttcggcaagaacggctacctggaaaccctgccattaggaaaggaagaaaaagaggccgtgaaggtgtttgccggattcggaggctttttcaacaactacaacaagaatcgggagaactacttcagtaccgaggagaagtccaccgccatcgccaacagaatcgtgaacgagaacttcagcaagcacttcagcaacgtggaaatcgttacaaagatccaaaaagaagtgccagagctgattcaaatcgtggaagctcagttcaagggttacgacaccatctttaccgtgaacggctacaacaccgccctgagccaggctggcatcgacacatacaacgaaatggtggccatctggaacaaggaggcaaacctgtacgctcaaaaagccggcaagctgccagacggccacccgctgaagaagaagcgtaactacctgctgagcgccctcttcaaacagatcggcagcgaaaaagaacacctgatccagatcgacagattcgacggcgacgaggaagtgatcgaagccctgactggcgtgaaaaagatgctgcaggaggccgacgtgttcgagaagctgaacatgctggtcgaggacatggaaaattgggattactccaagatctacctgtctgcccagagcctgagtaacgtgtccgtgttcctgaacaacctgtatgaagatgaacgggagaacagctggaactacctggataacgtgctgagagagaagtggcagattgaactgcagggcaaaaaaaagggaacagatctggaagaggccattagaaagaagaagcagagcttttactctatcgaggaacttcaggaggcagtgaacgccatcgaggaaaccgacaagtgctacaatgtgtctaaatggctgctgggagccatgaagagcgagagagtgatcgaggagaagaagaaagacgtggaggatttctgcacacagtggaagaacgagagaaacagcctcaaggaaaccgacatcaccgccctgaaggagtacctggagcagtggatcttcctggctaggtactgcaagagcttctacgccaatggcatcgaaaagaaagagaaggatgaggctttttaccacatcctggaggatgtgctgtacgtgctggacgaagtgatctacttctacaacaaggtgcggaactacgtgaccaaaaagccttacagtctggagaagatgcacctgaagttcggccacaacgagctggccaacggctggagcgtgaacaaggaagaaaattacggcaccgccatcctgagaagaaacggcaagtactacctggccatcaccaacagcctgaacaagaaaatgagcatccctacccagctggagagcacaggaaataattatgagaagatggtcctgaacgtttttcccaacgtgttccggatgatcccaaagtgcaccacaggcaggaacgacgtgaagtcatgcttcgagagaaaggaacccaacgagtacttcttcatcgacacccctgagttcgtgaacccctttaaggtcacacgggaggagtacgaactgaataagatcacctacgacggagttaagaagtggcagagcgactacagcaagaacacacaggacgaaaagggctataaggaagccgtgaccaagtggattcagttttgtatgcggttcctgcagtcttataagagcaccgccatatatgactacagcaccctgcagcaacctgaaaaatacgaaacagtggacagcttctatcatgatgtggaaaagatcctgtacgagtgccacttcgagtacgtgcccgctaacaagatcgagcagcttgaagaagagggaagaatcttcctgttccagatctacaacaaggatttttctgagaacagacggcctgatagcaagaaaaacctccacaccctgtactgggaggcgctgttctccgaagagaatagaaaggccaaggtgattcagctgaatggcaaggccgagatcttcagacgggagaaatcaatcgagcaccctatcgtgcataaggctggcgaggtgctggtgaacaagcggaccaaagatggcgaacctattcctgacgacatctacaaggacctgagcaactatttcaacggcagaaacgttacctctgagaaggaagagtacaaggagtgtctggacaaggtgtacaccagcaccaaaaagtacgatatcaccaaggacaaaagattcaccgagacaaagtacgagttccacgtgcctatcaccctgaactaccaggccgacggcgtgaagtacctgaatcagaagatcctgcacgtgctgcgggacaaccctgatgttaacatcatcggcctggatagaggcgaaagaaacctgatctcttatgttgtgctgaacagagagggcaagatcgtgaacaatcagcagggttctttcaacatcgtgggcaaaatggactaccagaaaaagctgtaccagaaggagaaaaaccgggataaagaacggaaaacgtggaaaaacatcgaaaccatcaaggacctgaaggagggctatatcagccaggtggtacacgagctgaccgatatggccatccggaataacgcgatcatcgtgatggaagatctgaatttcggattcaagcggggccggaccaaggtggaacggcaggtgtaccagaagtttgagctggccctgctgaagaagctgcactacctcgtgaccgacaagaccgagggagaagctatgctgaaacccggcggcgtgctgcaaggctaccagctggctagagaagtcaagaccctgaaagagatcggcaagcagtgcggctgtgtgttctacgtgccccctggctacacaagcaagatcgaccctacaaccggcttcgtcgacgtgttcaacatgtctggagttacaaaccgcgagaaaaagaaagcctttttcgaaaaatttgataacatgttctacgacgagaagagagacatgttcggcttcagcttcaattacgaaaagtttactacctaccagagcagctacagaaacgactggaccgtgtacagcaacggcagcaagtatgtgtggaactcccttaagagaacagacgagttaattgacgtgacaaaggagctcaagctgctgttcgagaagtacgccatcgattaccggaacgaagctctgtttgagcagatcatgagccaggatacagataagaacaacgccgacttctggaacaaactgttctggtacttccgggtgctgctgcggctgagaaatagcagcgacgaactggaccaaatcgtcagccctgtgctgaatcagaacggagagttcttcgaaacccctaagaaaatcacagagaagtcctacctgtctgattaccctatggacgccgatacaaacggcgcctaccacatcgccctgaagggcctgtacctgatccaggagaagatcgctgacgaatctgtggacctggacaacaagctgcctaaggacttctacaagatcagcaacgccgagtggttcatgtttagacagaaagaaaaatctagaaagcggacagcagacggctccgaatttgaaagccctaagaaaaagagaaaggtgggatccggcaaacctatccccaatcccctgctgggcctggacagcacctga

SEQ ID NO: 13 - protein, 1194 aa, unidentified
NIYENFTNMYQVNKTIRMGLKPICKTGENIAKFLEEDKETSDKYKIAKEVIDKENRAFIEDRLKDFSISGLDEYLELLKQKKNLTKNQNKMKKEISTQLTKIQNKMRDEISTQLKGFPQFDNKYKFKYITDKEDIEILKYFKDKKFITFFEEFNTNRKNVYSKENISTSIGHRIVHENLPKFISNFRILNKAIEAFGISKINEDFKNNGINVTVEELNKIDYFNKVLTQSGIDLYNNLIGILNQNINLYNQQQKVKKNKIGKLEILYKQILSKTDKVSFIEEFTEDNQLLECIDEYFKEKYSLITVDLNNLLENIDTYNLNGIFIKSDKSLGNISNYLYKDWWYISNLINEEYDYKHKNKVRDDKYYETRKKAIDKIKYFSIGHIDELLKDKNVPMVENYFKEKINLVVKEFNAYLNKFNEYKFINELKTDEIAVEIIKNLCDSIKNVQGIVKPLIITGNDKDDDFYVEINYIWDELNKFDKIYNMVRNYLTKKDYIEEKIRMMFSKSSFMDGWGKDYGTKKAHIVYHDKNYYLVIVDKKLKLEDIDKLYKPGGDTVHYVYNYQSTENGNIPRKFIYSKGKRFAPSVEKYNLPIEDVIEVYNNEYHTTDYEKKNPEIYKKSLTSLIDYFKIGVNRDMDFEKFDFRLKDSNEYKNIKEFYDNLETCCYKLQEEKVNFNVLEELSYSGKIYLFKIYNKDFSENSKGIPNLHTLYFKMLFDKENLENPIYKLSGKAKMFFRKGSLNLDKKTVDYDKKPIDKKENDKKIKNRRYKVDSFTLHMSIITNFQSYENKNVNETVNRALKYCDDVYAIGIDRGIRNLLYACVVNSKGEIVKQVPLNIINNKDYHNLLAEREEKKKNSRKNWKIIDNIRNLKEGYLSQAIHIITDLMVEYNAVLVLENLNFRFKEKQMKFESNVYQKFEKMLIDKLNFLVDKKLDKNANGGLFNAYQLTEKFTNFKDMKNQNGIIFYIPAWMTSKIDPVTGFTNLFYIKYESIEKAKEFFGKFKSIKFNKVDNYFEFEFDYNDFTDRAQGTRSKWTVCSFGPRIEGFRNPEKNNSWDGREIDITEKIKKLLDDYNVSLDKDIKAQIMDINTKDFFEKFIKYFKLVLQMRNSKTGTDIDYIISPVKNKQNEFFDSRKQNEKMPMDADANGAYNIARKGLMFIDIIKETEDKDLKMPKLFIKNKDWLNYVQKSDL

SEQ ID NO: 14 - protein, 1195 aa, unidentified
MNIYENFTNMYQVNKTIRMGLKPICKTGENIAKFLEEDKETSDKYKIAKEVIDKENRAFIEDRLKDFSISGLDEYLELLKQKKNLTKNQNKMKKEISTQLTKIQNKMRDEISTQLKGFPQFDNKYKFKYITDKEDIEILKYFKDKKFITFFEEFNTNRKNVYSKENISTSIGHRIVHENLPKFISNFRILNKAIEAFGISKINEDFKNNGINVTVEELNKIDYFNKVLTQSGIDLYNNLIGILNQNINLYNQQQKVKKNKIGKLEILYKQILSKTDKVSFIEEFTEDNQLLECIDEYFKEKYSLITVDLNNLLENIDTYNLNGIFIKSDKSLGNISNYLYKDWWYISNLINEEYDYKHKNKVRDDKYYETRKKAIDKIKYFSIGHIDELLKDKNVPMVENYFKEKINLVVKEFNAYLNKFNEYKFINELKTDEIAVEIIKNLCDSIKNVQGIVKPLIITGNDKDDDFYVEINYIWDELNKFDKIYNMVRNYLTKKDYIEEKIRMMFSKSSFMDGWGKDYGTKKAHIVYHDKNYYLVIVDKKLKLEDIDKLYKPGGDTVHYVYNYQSTENGNIPRKFIYSKGKRFAPSVEKYNLPIEDVIEVYNNEYHTTDYEKKNPEIYKKSLTSLIDYFKIGVNRDMDFEKFDFRLKDSNEYKNIKEFYDNLETCCYKLQEEKVNFNVLEELSYSGKIYLFKIYNKDFSENSKGIPNLHTLYFKMLFDKENLENPIYKLSGKAKMFFRKGSLNLDKKTVDYDKKPIDKKENDKKIKNRRYKVDSFTLHMSIITNFQSYENKNVNETVNRALKYCDDVYAIGIDRGIRNLLYACVVNSKGEIVKQVPLNIINNKDYHNLLAEREEKKKNSRKNWKIIDNIRNLKEGYLSQAIHIITDLMVEYNAVLVLENLNFRFKEKQMKFESNVYQKFEKMLIDKLNFLVDKKLDKNANGGLFNAYQLTEKFTNFKDMKNQNGIIFYIPAWMTSKIDPVTGFTNLFYIKYESIEKAKEFFGKFKSIKFNKVDNYFEFEFDYNDFTDRAQGTRSKWTVCSFGPRIEGFRNPEKNNSWDGREIDITEKIKKLLDDYNVSLDKDIKAQIMDINTKDFFEKFIKYFKLVLQMRNSKTGTDIDYIISPVKNKQNEFFDSRKQNEKMPMDADANGAYNIARKGLMFIDIIKETEDKDLKMPKLFIKNKDWLNYVQKSDL

SEQ ID NO: 15 - protein, 1232 aa, synthetic construct
MGNIYENFTNMYQVNKTIRMGLKPICKTGENIAKFLEEDKETSDKYKIAKEVIDKENRAFIEDRLKDFSISGLDEYLELLKQKKNLTKNQNKMKKEISTQLTKIQNKMRDEISTQLKGFPQFDNKYKFKYITDKEDIEILKYFKDKKFITFFEEFNTNRKNVYSKENISTSIGHRIVHENLPKFISNFRILNKAIEAFGISKINEDFKNNGINVTVEELNKIDYFNKVLTQSGIDLYNNLIGILNQNINLYNQQQKVKKNKIGKLEILYKQILSKTDKVSFIEEFTEDNQLLECIDEYFKEKYSLITVDLNNLLENIDTYNLNGIFIKSDKSLGNISNYLYKDWWYISNLINEEYDYKHKNKVRDDKYYETRKKAIDKIKYFSIGHIDELLKDKNVPMVENYFKEKINLVVKEFNAYLNKFNEYKFINELKTDEIAVEIIKNLCDSIKNVQGIVKPLIITGNDKDDDFYVEINYIWDELNKFDKIYNMVRNYLTKKDYIEEKIRMMFSKSSFMDGWGKDYGTKKAHIVYHDKNYYLVIVDKKLKLEDIDKLYKPGGDTVHYVYNYQSTENGNIPRKFIYSKGKRFAPSVEKYNLPIEDVIEVYNNEYHTTDYEKKNPEIYKKSLTSLIDYFKIGVNRDMDFEKFDFRLKDSNEYKNIKEFYDNLETCCYKLQEEKVNFNVLEELSYSGKIYLFKIYNKDFSENSKGIPNLHTLYFKMLFDKENLENPIYKLSGKAKMFFRKGSLNLDKKTVDYDKKPIDKKENDKKIKNRRYKVDSFTLHMSIITNFQSYENKNVNETVNRALKYCDDVYAIGIDRGIRNLLYACVVNSKGEIVKQVPLNIINNKDYHNLLAEREEKKKNSRKNWKIIDNIRNLKEGYLSQAIHIITDLMVEYNAVLVLENLNFRFKEKQMKFESNVYQKFEKMLIDKLNFLVDKKLDKNANGGLFNAYQLTEKFTNFKDMKNQNGIIFYIPAWMTSKIDPVTGFTNLFYIKYESIEKAKEFFGKFKSIKFNKVDNYFEFEFDYNDFTDRAQGTRSKWTVCSFGPRIEGFRNPEKNNSWDGREIDITEKIKKLLDDYNVSLDKDIKAQIMDINTKDFFEKFIKYFKLVLQMRNSKTGTDIDYIISPVKNKQNEFFDSRKQNEKMPMDADANGAYNIARKGLMFIDIIKETEDKDLKMPKLFIKNKDWLNYVQKSDLSRKRTADGSEFESPKKKRKVGSGKPIPNPLLGLDST

SEQ ID NO: 16 - DNA, 3588 bp, unidentified
  source 1..3588: mol_type = unassigned DNA
atgaacatttacgaaaattttactaatatgtatcaggtaaataagactataagaatggggttaaagccaatatgtaaaactggtgaaaatattgctaaatttcttgaggaagataaggaaacaagtgataaatacaagatagctaaagaagtaattgataaggaaaatagagcttttatagaggatagattaaaggatttttcaatttcagggttggatgaatatttggaattgcttaaacaaaaaaagaatttaaccaaaaatcaaaataaaatgaaaaaggaaatttcaacacagttaacaaaaatacaaaataaaatgagagatgaaatttcaacacagttaaaaggcttccctcaatttgataataaatataaattcaaatatattacagataaagaagatatagaaattttaaaatattttaaagataagaaatttattactttctttgaagaatttaatactaatagaaaaaatgtctactctaaagaaaatatttcaacttctattggacacagaattgttcacgaaaatcttccaaaatttatttcaaattttaggattttaaataaagcaatagaggcgtttggaataagtaaaataaatgaagattttaagaataatggaattaatgttacagttgaagaacttaataaaatagattattttaacaaggttttaactcaatcaggaatagatttgtataataatttgataggtattttaaatcaaaatataaatctatataatcaacaacagaaagtaaaaaagaataaaattggaaagttagaaatattatataagcaaattttaagtaaaacagataaagtatcgtttattgaagaatttactgaagataaccaacttttggaatgtattgatgaatattttaaagaaaaatatagtttgataactgtagatttaaataatttacttgaaaatattgatacttataatttgaatggtatctttattaaaagtgataagtccttgggaaatatatctaattatttatataaagattggtggtatatatcaaatcttataaacgaagaatacgattataaacataagaataaggtaagagatgataagtattatgaaacaagaaaaaaagctatagataagattaaatatttttccataggacatattgatgaattgttaaaagataaaaatgttcctatggtagaaaactatttcaaagaaaagataaatttagtagtaaaagaatttaatgcttatttaaacaaatttaatgaatataagtttataaatgagctaaaaactgatgaaattgctgtcgaaataataaaaaatttatgtgattcaataaagaatgtacaggggatagtaaagcctttaataattactggaaatgataaagacgatgatttttatgtggaaatcaattatatatgggacgagcttaataagtttgataaaatatataatatggttagaaattatcttacaaaaaaggattacatagaggaaaaaattagaatgatgttttcaaagagcagttttatggatggttggggaaaagattatggaacaaaaaaagcacatatagtttatcatgataaaaattattatttagtaatagtagacaagaaattaaaattagaggatatagataaattatataaaccaggtggagatactgtacattatgtatataattaccaatcaacagaaaatggaaatattcctagaaaattcatatattctaagggtaaaagatttgcaccatctgtagaaaaatataatttaccaatagaagatgttatcgaagtgtataacaatgaatatcatacaacagattacgaaaagaaaaatcctgaaatttacaagaaatcattaacatccttaattgattattttaaaataggggtaaatagggatatggattttgaaaaatttgattttagattaaaagattcaaacgaatacaaaaatataaaagaattttatgataatttggaaacttgttgctataagttacaagaagaaaaagttaattttaatgtacttgaagagctttcatatagtggaaaaatttatttatttaaaatatacaataaggatttttctgaaaatagcaaaggaatacctaatcttcatactttatattttaaaatgctatttgacaaagaaaaccttgaaaatccgatttataaacttagtggaaaggctaaaatgttttttagaaagggtagtcttaatttagacaaaaaaactgttgattatgataaaaagccaatagataagaaagaaaatgacaaaaaaattaaaaatagaagatataaagttgatagttttacattacatatgtcaattattacgaactttcagtcatatgaaaataaaaatgtaaatgaaactgtaaatagggctttaaaatattgtgatgatgtttatgccataggtatagacagaggaataagaaatttattatatgcttgtgtagtaaattcaaagggagaaatagtaaaacaagttcctttaaatattataaataataaagattatcacaatttacttgcagaaagagaagagaagaaaaagaatagtaggaaaaattggaaaatcattgataatataaggaatttaaaggaaggctatttaagtcaggccatacatataataactgaccttatggttgaatataatgctgtacttgttttagagaatttgaattttagatttaaagaaaaacaaatgaaatttgaaagtaatgtttatcaaaaatttgaaaagatgcttattgataaattgaatttcttagttgataaaaagcttgataagaacgccaatggtggattgtttaatgcgtatcaattaacagaaaaatttacaaactttaaagatatgaaaaatcaaaatggtataatattttatattcctgcttggatgacaagcaaaattgacccagttacaggatttacaaatttattctatattaaatatgagagtattgaaaaggctaaagagttttttggtaagtttaaatcaataaaatttaataaggtagacaactattttgaatttgaatttgattataatgattttactgacagagctcaaggtacaaggtctaaatggacagtttgtagttttggccctagaattgaaggttttagaaatcctgaaaaaaataatagttgggatggtagagaaatagatataacagagaaaattaaaaaattacttgatgattataatgtatctttagataaagatattaaagctcaaattatggatataaatactaaggatttctttgaaaaatttattaaatattttaaacttgtattgcaaatgagaaacagtaaaacaggtacagatattgattatatcatttctccggttaaaaataagcaaaatgaattttttgacagtagaaagcaaaatgaaaaaatgcctatggatgcagatgcaaatggtgcttataatattgctagaaaaggcttaatgtttattgatataataaaagaaactgaagataaagatttaaagatgcctaaattgttcattaaaaataaagattggttaaattatgtacaaaagagtgatttgtaa

SEQ ID NO: 17 - DNA, 3582 bp, synthetic construct
  source 1..3582: mol_type = other DNA
aatatctatgagaacttcaccaacatgtaccaggtgaacaagacaatccgcatgggcctgaagcctatctgtaaaaccggagaaaacatcgccaagttcctggaggaggacaaggaaaccagcgacaagtacaagatcgccaaggaggtcatcgacaaggagaacagagcctttatcgaggacagactgaaggacttcagcatcagcggcctggacgagtacctggaactgctgaagcagaagaaaaacctgacaaagaaccagaacaagatgaaaaaggaaatctccacccagctgacaaagatccagaacaagatgcgggacgagatatcgacacagctgaagggcttccctcagttcgataacaaatacaagttcaaatatatcacagacaaggaggacatcgaaatcctcaagtacttcaaggataagaagttcattacattctttgaggaatttaataccaatcggaaaaacgtgtacagcaaggaaaacatcagcacctctatcggccatagaatcgtgcacgagaacctgccaaagttcatcagcaacttcagaatcctgaataaggccatcgaggccttcggcatctctaaaatcaatgaggacttcaagaacaatggcatcaacgtgaccgtagaagaactgaacaagatcgactacttcaacaaggtcctgacacagagcggcattgacctgtacaacaacctgattggcatcctgaaccagaacatcaacctgtacaatcagcagcagaaggtgaagaagaacaaaatcggaaagctggaaatcctgtacaagcaaatcttgtccaaaaccgacaaggtgtctttcattgaggagttcaccgaggacaaccagctgctggagtgcatcgacgagtactttaaagagaaatacagcctgatcaccgtggacctgaacaacctgcttgaaaatatcgacacctacaatctcaacggcatcttcatcaaatctgataaaagcctgggcaacatcagcaactacctgtacaaggattggtggtacatcagcaacctgatcaacgaagaatacgactacaagcacaagaacaaggtcagagatgataagtactacgagacaagaaagaaggccatcgacaagatcaagtacttctctatcggacacatcgatgagctgctgaaggacaagaacgttccaatggtggaaaactacttcaaggagaagatcaacctggtcgtgaaggagttcaatgcttatctgaacaagttcaatgaatataaattcatcaacgagctgaaaacagacgagatcgccgtggaaatcatcaagaacctgtgcgacagcatcaagaacgtgcagggcatcgtgaagcccctgatcatcaccggcaacgacaaggatgatgatttttacgtggagatcaactacatctgggatgagcttaacaagttcgacaaaatctacaacatggtcaggaattacctaaccaagaaggactacatcgaggaaaagatcagaatgatgttttccaagagcagctttatggacggctggggcaaggactacggcaccaagaaggcccacatcgtgtaccacgacaagaactactacctggtgatcgtggacaagaagctgaaactggaagatatcgacaaactatacaagccaggcggcgacacagttcactacgtgtacaactaccagtctaccgagaacggaaacatccctcggaagttcatctactctaagggcaagcggttcgcccctagcgtggaaaaatataacctgcctattgaagatgtgattgaggtgtacaacaacgagtaccacaccaccgactatgagaaaaagaaccctgagatatacaaaaagtccctgaccagcctgatcgactatttcaagatcggcgtgaacagagatatggacttcgagaagtttgattttcggctaaaggactccaacgaatacaagaacatcaaggagttctacgataacctggagacatgctgctacaagctgcaggaggaaaaggtgaacttcaacgtgctggaggaactgagctacagcggaaagatctacctgttcaagatctacaacaaagatttcagcgagaatagcaaaggcatccctaacctgcataccctgtacttcaaaatgctgttcgacaaagagaacctggagaaccccatctacaagctgtctggaaaagctaagatgtttttcagaaagggcagcctgaacctggacaaaaaaaccgttgactatgacaaaaaacctatcgataagaaggaaaacgacaaaaaaatcaagaataggcggtacaaggtggacagcttcaccctgcacatgagcatcatcaccaacttccagagctacgagaacaagaacgttaatgagactgtgaaccgggccctgaagtactgcgacgacgtgtacgccatcggcatcgaccgcggaatccggaacctgctgtacgcttgtgtggtgaacagcaagggcgagatcgtgaagcaagtgcccctcaacatcattaacaataaggattaccacaacctgctggccgagagagaagaaaagaagaaaaacagcagaaagaattggaagatcatagacaacatcagaaacctgaaggaaggctacctgagccaggccatccacatcatcaccgacctgatggtggaatacaacgccgtgctggtgctggagaacctgaatttcagattcaaggagaagcagatgaagtttgaaagcaatgtgtaccaaaaattcgaaaaaatgctgatcgacaagctgaatttcctggtcgataaaaaactggacaagaatgccaatggcggactgtttaacgcctatcagctgacagagaagttcaccaactttaaggatatgaagaatcagaacggcatcatcttctacatccccgcctggatgacaagcaagatcgatcccgtgaccggcttcacaaacctgttttatatcaaatacgagagcatcgagaaggcaaaggagttcttcggcaagtttaagtctatcaagttcaataaggtggacaattatttcgagttcgagttcgactacaacgactttaccgacagagctcaaggcaccagaagcaagtggaccgtgtgtagcttcggtcctcggatcgagggcttcagaaaccccgagaaaaacaattcctgggacggcagagaaatcgacatcacagagaagatcaagaagctgctggatgactacaatgtgagcctggacaaagacatcaaagcccagatcatggacatcaacaccaaggatttcttcgagaagttcatcaagtacttcaagctggtgctgcagatgagaaacagcaagaccggcaccgacatcgattacattatctcccctgtgaagaacaagcagaacgagtttttcgactccagaaagcagaacgagaagatgcctatggacgctgatgccaacggcgcctacaacatcgctagaaaggggctgatgttcatcgatatcatcaaggaaacagaggacaaggacctgaaaatgcctaagctgttcataaagaacaaggattggctgaactatgtgcagaaatcagatctg

SEQ ID NO: 18 - DNA, 3699 bp, synthetic construct
  source 1..3699: mol_type = other DNA
atgggcaatatctatgagaacttcaccaacatgtaccaggtgaacaagacaatccgcatgggcctgaagcctatctgtaaaaccggagaaaacatcgccaagttcctggaggaggacaaggaaaccagcgacaagtacaagatcgccaaggaggtcatcgacaaggagaacagagcctttatcgaggacagactgaaggacttcagcatcagcggcctggacgagtacctggaactgctgaagcagaagaaaaacctgacaaagaaccagaacaagatgaaaaaggaaatctccacccagctgacaaagatccagaacaagatgcgggacgagatatcgacacagctgaagggcttccctcagttcgataacaaatacaagttcaaatatatcacagacaaggaggacatcgaaatcctcaagtacttcaaggataagaagttcattacattctttgaggaatttaataccaatcggaaaaacgtgtacagcaaggaaaacatcagcacctctatcggccatagaatcgtgcacgagaacctgccaaagttcatcagcaacttcagaatcctgaataaggccatcgaggccttcggcatctctaaaatcaatgaggacttcaagaacaatggcatcaacgtgaccgtagaagaactgaacaagatcgactacttcaacaaggtcctgacacagagcggcattgacctgtacaacaacctgattggcatcctgaaccagaacatcaacctgtacaatcagcagcagaaggtgaagaagaacaaaatcggaaagctggaaatcctgtacaagcaaatcttgtccaaaaccgacaaggtgtctttcattgaggagttcaccgaggacaaccagctgctggagtgcatcgacgagtactttaaagagaaatacagcctgatcaccgtggacctgaacaacctgcttgaaaatatcgacacctacaatctcaacggcatcttcatcaaatctgataaaagcctgggcaacatcagcaactacctgtacaaggattggtggtacatcagcaacctgatcaacgaagaatacgactacaagcacaagaacaaggtcagagatgataagtactacgagacaagaaagaaggccatcgacaagatcaagtacttctctatcggacacatcgatgagctgctgaaggacaagaacgttccaatggtggaaaactacttcaaggagaagatcaacctggtcgtgaaggagttcaatgcttatctgaacaagttcaatgaatataaattcatcaacgagctgaaaacagacgagatcgccgtggaaatcatcaagaacctgtgcgacagcatcaagaacgtgcagggcatcgtgaagcccctgatcatcaccggcaacgacaaggatgatgatttttacgtggagatcaactacatctgggatgagcttaacaagttcgacaaaatctacaacatggtcaggaattacctaaccaagaaggactacatcgaggaaaagatcagaatgatgttttccaagagcagctttatggacggctggggcaaggactacggcaccaagaaggcccacatcgtgtaccacgacaagaactactacctggtgatcgtggacaagaagctgaaactggaagatatcgacaaactatacaagccaggcggcgacacagttcactacgtgtacaactaccagtctaccgagaacggaaacatccctcggaagttcatctactctaagggcaagcggttcgcccctagcgtggaaaaatataacctgcctattgaagatgtgattgaggtgtacaacaacgagtaccacaccaccgactatgagaaaaagaaccctgagatatacaaaaagtccctgaccagcctgatcgactatttcaagatcggcgtgaacagagatatggacttcgagaagtttgattttcggctaaaggactccaacgaatacaagaacatcaaggagttctacgataacctggagacatgctgctacaagctgcaggaggaaaaggtgaacttcaacgtgctggaggaactgagctacagcggaaagatctacctgttcaagatctacaacaaagatttcagcgagaatagcaaaggcatccctaacctgcataccctgtacttcaaaatgctgttcgacaaagagaacctggagaaccccatctacaagctgtctggaaaagctaagatgtttttcagaaagggcagcctgaacctggacaaaaaaaccgttgactatgacaaaaaacctatcgataagaaggaaaacgacaaaaaaatcaagaataggcggtacaaggtggacagcttcaccctgcacatgagcatcatcaccaacttccagagctacgagaacaagaacgttaatgagactgtgaaccgggccctgaagtactgcgacgacgtgtacgccatcggcatcgaccgcggaatccggaacctgctgtacgcttgtgtggtgaacagcaagggcgagatcgtgaagcaagtgcccctcaacatcattaacaataaggattaccacaacctgctggccgagagagaagaaaagaagaaaaacagcagaaagaattggaagatcatagacaacatcagaaacctgaaggaaggctacctgagccaggccatccacatcatcaccgacctgatggtggaatacaacgccgtgctggtgctggagaacctgaatttcagattcaaggagaagcagatgaagtttgaaagcaatgtgtaccaaaaattcgaaaaaatgctgatcgacaagctgaatttcctggtcgataaaaaactggacaagaatgccaatggcggactgtttaacgcctatcagctgacagagaagttcaccaactttaaggatatgaagaatcagaacggcatcatcttctacatccccgcctggatgacaagcaagatcgatcccgtgaccggcttcacaaacctgttttatatcaaatacgagagcatcgagaaggcaaaggagttcttcggcaagtttaagtctatcaagttcaataaggtggacaattatttcgagttcgagttcgactacaacgactttaccgacagagctcaaggcaccagaagcaagtggaccgtgtgtagcttcggtcctcggatcgagggcttcagaaaccccgagaaaaacaattcctgggacggcagagaaatcgacatcacagagaagatcaagaagctgctggatgactacaatgtgagcctggacaaagacatcaaagcccagatcatggacatcaacaccaaggatttcttcgagaagttcatcaagtacttcaagctggtgctgcagatgagaaacagcaagaccggcaccgacatcgattacattatctcccctgtgaagaacaagcagaacgagtttttcgactccagaaagcagaacgagaagatgcctatggacgctgatgccaacggcgcctacaacatcgctagaaaggggctgatgttcatcgatatcatcaaggaaacagaggacaaggacctgaaaatgcctaagctgttcataaagaacaaggattggctgaactatgtgcagaaatcagatctgtctagaaagcggacagcagacggctccgaatttgaaagccctaagaaaaagagaaaggtgggatccggcaaacctatccccaatcccctgctgggcctggacagcacctga

SEQ ID NO: 19 - protein, 1244 aa, unidentified
EISNRFTNKYQVSKTLRFRLEPTGGTDDLLCQAQIIEGDERRNKEAITMKQILDNCHKQIIERVLSDFNFKEHSLEEFFKVYTRNDDDREKDIENLQAKMRKEIAAAFTKQDVTKLFSSKFKDFVERGLIKYASNEKERNIVSRFKGFATYFTGFNTNRLNMYSEEAKSTAISFRLINQNLIKFIDNILVYKKVSQTLPSDVLSNIYIDFKAIINTSSLEEFFSINNYNNILTQKQIEIFNAVIGGKKDKDEKIITKGFNQYINEYNQTNKNIRLPKMMRLFNQILSDREGVSARPEPFNNANETISSVRDCFTNEISKQITILSETTSKIESFDIDRIYIKGGEDLRALSNSIYGYFNYIHDRIADKWKHNNPQGKKSPESYQKNLNAYLKGIKSVSLHSIANICGDNKVIEYFRNLGAENTVDFQRENVVSLIDNKYNCASNLLSDAQITDEELRTNSRSIKDLLDAVKSAQRFFRLLCGSGNEPDKDHSFYDEYTPAFEALENSINPLYNKVRSFVTKKDFSTDKFKLNFDSSSFLSGWAKKSEYEKSSAFIFIRDNQYYLGINKCLSKEDIAYLEDSTSSSDTKRVVYMFQKVDATNIPRIFIRSKGSNLAPAVNEFQLPIETILDIYDNKFFTTSYQKKDRTKWKESLTKLIDYYKLGFSQHKSYADFDLKWKASSEYNDINDFLADVQRFCYRIEFININWDKLIEFTEDGKFYLFRIANKDLSGNSTGLPNLHTIYWKMLFDESNLKDIVYKLSGNAEVFMRYNSLKNPIVHKAGVEIKNKCPFTEKKTSIFDYDIIKDRRYTKDQLELHVPILMNFKSPSAAKGKAFNKECLEYIRNNGIKHIIGIDRGERNLLYMVITDLDGNIVEQKSLNQIASNPKLPLFRQDYNKLLKTKADANAQARRDWETIDTVKEIKFGFLSQIVHEIAMAIIKYDAIVVLENLNRGFMQKRGLENNVYQKFEQMLLDKLSYYVDKTKHPEEAGGALHAYQLSDTYANFNSLSKNAMVRQSGFVFYIPAWLTSKIDPVTGFASFLKFHRDDSMATIKSTISKFDCFKYDKECDMFHIRIDYNKFSTSCSGGQRKWDLFTFGDRILAERNTMQNSRYVYQTVNLTSEFKNLFATKDIDISGNLKDSICKIEDVGFFRKLSQLLSLTLQLRNSNAETGEDFLISPVADKDGNFFDSRNCPDSLPKDADANGAYNIARKGLMLVEQLKRCKDVSKFKPAIKNEDWLDYVQR

SEQ ID NO: 20 - protein, 1245 aa, unidentified
MEISNRFTNKYQVSKTLRFRLEPTGGTDDLLCQAQIIEGDERRNKEAITMKQILDNCHKQIIERVLSDFNFKEHSLEEFFKVYTRNDDDREKDIENLQAKMRKEIAAAFTKQDVTKLFSSKFKDFVERGLIKYASNEKERNIVSRFKGFATYFTGFNTNRLNMYSEEAKSTAISFRLINQNLIKFIDNILVYKKVSQTLPSDVLSNIYIDFKAIINTSSLEEFFSINNYNNILTQKQIEIFNAVIGGKKDKDEKIITKGFNQYINEYNQTNKNIRLPKMMRLFNQILSDREGVSARPEPFNNANETISSVRDCFTNEISKQITILSETTSKIESFDIDRIYIKGGEDLRALSNSIYGYFNYIHDRIADKWKHNNPQGKKSPESYQKNLNAYLKGIKSVSLHSIANICGDNKVIEYFRNLGAENTVDFQRENVVSLIDNKYNCASNLLSDAQITDEELRTNSRSIKDLLDAVKSAQRFFRLLCGSGNEPDKDHSFYDEYTPAFEALENSINPLYNKVRSFVTKKDFSTDKFKLNFDSSSFLSGWAKKSEYEKSSAFIFIRDNQYYLGINKCLSKEDIAYLEDSTSSSDTKRVVYMFQKVDATNIPRIFIRSKGSNLAPAVNEFQLPIETILDIYDNKFFTTSYQKKDRTKWKESLTKLIDYYKLGFSQHKSYADFDLKWKASSEYNDINDFLADVQRFCYRIEFININWDKLIEFTEDGKFYLFRIANKDLSGNSTGLPNLHTIYWKMLFDESNLKDIVYKLSGNAEVFMRYNSLKNPIVHKAGVEIKNKCPFTEKKTSIFDYDIIKDRRYTKDQLELHVPILMNFKSPSAAKGKAFNKECLEYIRNNGIKHIIGIDRGERNLLYMVITDLDGNIVEQKSLNQIASNPKLPLFRQDYNKLLKTKADANAQARRDWETIDTVKEIKFGFLSQIVHEIAMAIIKYDAIVVLENLNRGFMQKRGLENNVYQKFEQMLLDKLSYYVDKTKHPEEAGGALHAYQLSDTYANFNSLSKNAMVRQSGFVFYIPAWLTSKIDPVTGFASFLKFHRDDSMATIKSTISKFDCFKYDKECDMFHIRIDYNKFSTSCSGGQRKWDLFTFGDRILAERNTMQNSRYVYQTVNLTSEFKNLFATKDIDISGNLKDSICKIEDVGFFRKLSQLLSLTLQLRNSNAETGEDFLISPVADKDGNFFDSRNCPDSLPKDADANGAYNIARKGLMLVEQLKRCKDVSKFKPAIKNEDWLDYVQR

SEQ ID NO: 21 - protein, 1282 aa, synthetic construct
MGEISNRFTNKYQVSKTLRFRLEPTGGTDDLLCQAQIIEGDERRNKEAITMKQILDNCHKQIIERVLSDFNFKEHSLEEFFKVYTRNDDDREKDIENLQAKMRKEIAAAFTKQDVTKLFSSKFKDFVERGLIKYASNEKERNIVSRFKGFATYFTGFNTNRLNMYSEEAKSTAISFRLINQNLIKFIDNILVYKKVSQTLPSDVLSNIYIDFKAIINTSSLEEFFSINNYNNILTQKQIEIFNAVIGGKKDKDEKIITKGFNQYINEYNQTNKNIRLPKMMRLFNQILSDREGVSARPEPFNNANETISSVRDCFTNEISKQITILSETTSKIESFDIDRIYIKGGEDLRALSNSIYGYFNYIHDRIADKWKHNNPQGKKSPESYQKNLNAYLKGIKSVSLHSIANICGDNKVIEYFRNLGAENTVDFQRENVVSLIDNKYNCASNLLSDAQITDEELRTNSRSIKDLLDAVKSAQRFFRLLCGSGNEPDKDHSFYDEYTPAFEALENSINPLYNKVRSFVTKKDFSTDKFKLNFDSSSFLSGWAKKSEYEKSSAFIFIRDNQYYLGINKCLSKEDIAYLEDSTSSSDTKRVVYMFQKVDATNIPRIFIRSKGSNLAPAVNEFQLPIETILDIYDNKFFTTSYQKKDRTKWKESLTKLIDYYKLGFSQHKSYADFDLKWKASSEYNDINDFLADVQRFCYRIEFININWDKLIEFTEDGKFYLFRIANKDLSGNSTGLPNLHTIYWKMLFDESNLKDIVYKLSGNAEVFMRYNSLKNPIVHKAGVEIKNKCPFTEKKTSIFDYDIIKDRRYTKDQLELHVPILMNFKSPSAAKGKAFNKECLEYIRNNGIKHIIGIDRGERNLLYMVITDLDGNIVEQKSLNQIASNPKLPLFRQDYNKLLKTKADANAQARRDWETIDTVKEIKFGFLSQIVHEIAMAIIKYDAIVVLENLNRGFMQKRGLENNVYQKFEQMLLDKLSYYVDKTKHPEEAGGALHAYQLSDTYANFNSLSKNAMVRQSGFVFYIPAWLTSKIDPVTGFASFLKFHRDDSMATIKSTISKFDCFKYDKECDMFHIRIDYNKFSTSCSGGQRKWDLFTFGDRILAERNTMQNSRYVYQTVNLTSEFKNLFATKDIDISGNLKDSICKIEDVGFFRKLSQLLSLTLQLRNSNAETGEDFLISPVADKDGNFFDSRNCPDSLPKDADANGAYNIARKGLMLVEQLKRCKDVSKFKPAIKNEDWLDYVQRSRKRTADGSEFESPKKKRKVGSGKPIPNPLLGLDST

SEQ ID NO: 22 - DNA, 3738 bp, unidentified
  source 1..3738: mol_type = unassigned DNA
atggaaatttcgaaccgattcacaaacaagtatcaagtaagcaagaccctccgctttcgccttgagccaaccggaggtactgatgatttactttgccaagcacaaatcatcgagggagacgagcgccgcaataaagaggctataacaatgaaacagattttggacaattgtcacaaacagataattgagcgcgtattgtccgactttaattttaaagagcattctcttgaagagtttttcaaagtgtataccagaaacgatgatgaccgcgaaaaggacattgaaaatctccaagcaaaaatgcgcaaagaaatagccgccgccttcaccaaacaggatgttacgaaacttttctcaagcaaattcaaggattttgttgaaagaggcttgattaaatatgcatcaaacgagaaggaacgcaacatcgtttcccgcttcaaaggttttgccacttactttacagggttcaataccaatagactgaatatgtactcagaagaagcaaaatccacagctatatcattcagattaattaatcaaaacttgataaagttcatagacaacatccttgtatataaaaaagtgtctcaaacgttgccttcagatgtgctatcaaacatttatatagactttaaggcaatcatcaacacatcaagtcttgaagaattcttctccataaacaactacaataacatactcacccagaaacagattgagattttcaatgcagttatcggaggtaaaaaagacaaggatgaaaaaataataaccaaaggattcaaccaatatataaacgaatacaaccagaccaataaaaacatccgtctgcctaagatgatgcggttattcaatcaaatcctaagcgacagagaaggtgtttctgcaagaccagagccattcaataacgcgaacgagacaatcagttccgtccgtgattgttttacaaacgaaatatcaaaacaaataacgatattgtctgaaacaacatccaaaattgaatcattcgacattgatagaatttacattaagggcggagaagatctgagagcattatccaacagtatatatggatatttcaattatatccatgaccgtatcgcagacaaatggaaacacaacaatcctcagggcaaaaagagccccgaaagctaccaaaaaaacctcaacgcatatctgaaaggcataaaaagcgtctctttacacagtattgcaaacatctgtggtgacaacaaagttattgagtatttcaggaatcttggtgcagaaaacactgttgatttccaaagagagaacgttgtatcattaatcgacaacaaatacaactgcgcttcaaatcttttatccgacgcccaaattacggatgaagaacttcgcacaaacagtcgctcaattaaagacttgcttgacgccgtcaagagtgcccaacgatttttccgtctactgtgcggttctggcaacgaaccagacaaagaccactctttttatgacgagtatacaccagcatttgaagcacttgagaattcaataaatcccctatataacaaagtcaggagttttgtaaccaaaaaagatttctccaccgataaattcaaattgaatttcgacagcagcagctttctatccggttgggcaaagaaatcagaatatgagaagagttctgcatttatatttattcgcgacaatcaatattacttaggaataaacaaatgccttagcaaagaagacattgcctaccttgaggactcaacaagctcatcagatacaaaaagagtggtatatatgttccaaaaagtggacgccacgaatattcccagaatattcatccgttccaaaggttccaatttagctcctgctgtcaacgaattccaactgccgatagaaaccattcttgacatttatgacaataagttcttcactaccagttatcagaaaaaagaccggactaaatggaaagaatcattgaccaaactcattgactattacaagcttggattcagccagcacaagtcatacgcagatttcgacttaaaatggaaagcatccagtgaatataacgacataaatgactttcttgcagacgtacagagattctgctacagaatcgaatttataaatatcaattgggacaagctgatagaattcacagaagatggcaaattttacctattccgcattgcaaataaagatttatcaggcaatagcacaggtctgcccaatttgcacacgatttattggaaaatgctttttgacgaaagcaacctcaaagatattgtctataaattgtcgggcaatgcggaagtctttatgcgctataattcattaaaaaatccaattgtgcataaagcgggagtggagattaaaaacaaatgcccttttactgaaaaaaagacaagcatatttgactacgacattataaaagaccgtcgctatacaaaagatcagcttgaactgcatgttccaatcctaatgaacttcaaaagcccatcggcagcaaaaggcaaagctttcaacaaagaatgcttggaatacataagaaataatggtataaagcatattataggaatagaccgaggtgaacggaatctactttatatggttataacagaccttgacggcaacatcgttgagcaaaagtctttgaaccaaattgcgagcaatccgaaattgcctcttttcagacaagactacaacaagctgctgaagacaaaggctgatgcaaacgcacaagcacgtcgtgattgggaaacaatagacaccgtaaaggagataaaattcggcttcttgagtcagattgtacatgagatagcaatggctatcataaaatacgatgcaattgttgttttggagaatctgaacagagggtttatgcagaaacgaggtcttgaaaacaacgtctatcagaaattcgaacaaatgctgcttgacaagttgagctactatgtcgacaaaacgaaacatccggaagaggccggaggagctttgcacgcatatcagctctctgacacttacgcgaacttcaattctctgtcgaagaatgcgatggtgcgacagtcgggttttgttttctatattcctgcatggcttacaagcaaaatagaccccgtcacaggattcgcctcctttttgaaatttcacagagatgacagtatggcaacaatcaaatctacaatttcaaagtttgattgtttcaaatacgacaaggaatgcgacatgttccacatccgcattgactataacaagtttagcacaagctgcagcggaggtcaacgcaaatgggacttgttcacttttggcgatcgaatcttggcagaacgcaatacaatgcaaaacagcagatatgtttaccaaacagtcaatttaacttctgaattcaaaaacttatttgccacaaaggatatcgacatttcaggcaacctgaaggactctatatgcaaaattgaggatgttggctttttcagaaaactaagccaactcttgtcactcacgcttcaattacgcaacagcaatgctgaaacaggagaagacttcttgatttccccagtagctgacaaagatggcaatttcttcgattcaagaaactgtcccgactctctcccaaaagacgcagatgccaatggcgcatacaacattgctaggaagggattaatgcttgtcgagcaattgaagagatgcaaagatgtatcaaaattcaagcccgcgataaaaaacgaggactggttagactatgttcaacgctga

SEQ ID NO: 23 - DNA, 3732 bp, synthetic construct
  source 1..3732: mol_type = other DNA
gaaatcagtaatcggtttacaaacaagtaccaggtgtctaagaccctgcggttcagactggagcctacaggcgggaccgatgacctgctgtgccaggcccagatcatcgagggcgatgagcggcgcaacaaagaagccatcaccatgaaacagatcctcgacaactgtcacaagcagatcatcgaaagagtgctgtccgacttcaacttcaaagagcactccctggaagagttctttaaggtgtacacacggaacgacgatgacagagagaaggatatcgagaacctgcaggcaaagatgcgcaaggaaatcgccgccgcctttactaagcaagacgtgacaaaactgttttcttccaagtttaaagactttgtcgaaaggggtctgatcaagtacgccagcaacgagaaggagcggaatatcgtgtcccggttcaagggctttgccacatacttcaccggcttcaacacaaaccgcctgaacatgtacagcgaggaagccaaatctacggccattagcttccggctgatcaaccagaacctcatcaaattcatcgacaatatcctggtgtacaagaaggtgtctcagaccctcccttctgatgtcctgagcaacatctacatcgacttcaaggccatcatcaataccagcagcctggaggagttcttctccatcaacaactacaacaacatcctgacccagaagcagatcgagatcttcaacgctgtgatcggcggaaagaaggataaggatgagaaaattatcacaaagggcttcaaccagtacatcaatgaatataatcagaccaacaagaatatcagactgccaaagatgatgagactgttcaatcagatactgagcgaccgggaaggcgtgtcagctagacctgagcccttcaacaacgccaacgagacaatcagctccgtgagagactgttttacaaacgaaatcagcaagcagatcaccatcctgtctgaaaccaccagtaagatcgagagcttcgacatcgatagaatctacatcaagggcggagaggacctgcgggccctgagcaacagcatctacggctacttcaactacatccacgatagaatcgctgataagtggaagcacaacaatcctcagggcaagaagagccccgagagctaccaaaagaatctgaacgcctacctgaagggcataaagagcgtgagcctgcattctatcgccaacatctgtggcgacaacaaggtgatcgaatattttagaaatctcggcgccgagaacacagtggattttcagagagaaaacgtggtgtccctaattgacaacaaatacaactgtgcctcaaacctgctgtccgacgcccaaatcaccgacgaggagctgaggaccaacagcagaagcatcaaggatctgctcgacgccgtgaagagtgcccagagattcttcagactgctgtgcggttctggcaatgagcctgataaagaccacagcttttatgacgagtacacccctgctttcgaggccctggaaaacagcatcaaccccctgtacaacaaggtccgcagcttcgtgaccaaaaaggacttcagcacagacaagttcaaactgaacttcgacagcagcagcttcctgagcggatgggccaagaaaagcgagtacgagaagagcagcgctttcatcttcatcagggataatcagtactacctgggaattaataagtgcctgagtaaagaggacatcgcctacctggaggacagcacctctagcagcgacacaaagagagtggtgtacatgtttcagaaggtggatgccaccaatatcccaagaatcttcatcagatccaagggcagcaacctggcccctgctgtgaacgagttccagctgcctatcgaaaccatcctggatatctacgacaacaagttcttcaccaccagttaccagaagaaggatagaaccaaatggaaggaaagcctgaccaagctgatcgactactacaagctgggctttagccagcacaagtcctatgccgatttcgatttaaagtggaaagccagctcagaatacaatgacatcaatgatttcctggccgacgtgcagagattctgctacagaattgagttcatcaatatcaattgggacaagctcatcgagttcacagaggacggcaagttctacctgtttagaatcgccaacaaagacctgtctggcaacagcactggcctgcccaatctgcacaccatctactggaagatgctgttcgacgagagcaacctgaaggacatcgtgtacaagctgagcggcaacgctgaggtgtttatgcgctacaacagcctgaagaaccccattgtgcacaaggccggagtggaaatcaagaataagtgtcctttcaccgagaagaaaaccagcatctttgactacgacattatcaaggaccgcagatacaccaaggaccagctggaactgcatgtgcctatcctgatgaacttcaagtctccatctgccgctaaaggcaaagcctttaacaaggagtgcctggaatacatcagaaacaacggcatcaagcacatcatcggcatcgacagaggagagcggaatctgctttacatggtgatcacagacctggacggcaacatcgtggaacagaagtctctgaaccagatcgcctccaatccaaagctgcctctgttcagacaggactacaacaagctgctgaaaaccaaagctgacgccaacgcacaagccagaagagactgggagacaatagacaccgtgaaggagattaagttcggcttcctgagccagatcgtgcacgagatcgctatggccatcatcaagtacgacgccattgtggtcctggaaaacctgaacagaggcttcatgcaaaaacggggcctggaaaacaacgtgtatcagaagttcgagcaaatgctcctcgataaactgagctactatgtcgacaagaccaaacaccctgaggaagctggcggagccctgcacgcctatcagttaagcgatacctacgccaacttcaattccttgagcaagaacgctatggtgagacagtctggcttcgtgttctacatccccgcctggctgaccagcaagatcgatcctgtgaccggcttcgcctctttcctgaagttccacagagatgatagcatggccaccatcaagagcaccatctccaaattcgactgcttcaagtacgacaaggaatgcgacatgttccacatcagaatagattacaacaaatttagcacttcatgcagcggtggccagcggaagtgggatctgttcacattcggagacagaatcctggccgagagaaacaccatgcagaacagtagatacgtttaccagacagttaacctgacctctgagttcaagaacctgttcgccacaaaggatatcgatataagcgggaacctgaaggatagcatctgcaagatcgaggacgtgggcttcttccggaagctgagccagctgctgagcctgacactacagcttcggaacagcaacgctgaaaccggagaagatttcctgatcagccctgtggccgacaaggacggcaacttctttgacagcagaaactgccccgacagcctgccaaaggatgcagacgcgaatggcgcttataacattgccaggaagggcctgatgctggtggagcaactgaagcggtgcaaggacgtgagcaagttcaagcctgctatcaagaacgaggactggctggactacgtgcagcgg

SEQ ID NO: 24 - DNA, 3849 bp, synthetic construct
  source 1..3849: mol_type = other DNA
atgggcgaaatcagtaatcggtttacaaacaagtaccaggtgtctaagaccctgcggttcagactggagcctacaggcgggaccgatgacctgctgtgccaggcccagatcatcgagggcgatgagcggcgcaacaaagaagccatcaccatgaaacagatcctcgacaactgtcacaagcagatcatcgaaagagtgctgtccgacttcaacttcaaagagcactccctggaagagttctttaaggtgtacacacggaacgacgatgacagagagaaggatatcgagaacctgcaggcaaagatgcgcaaggaaatcgccgccgcctttactaagcaagacgtgacaaaactgttttcttccaagtttaaagactttgtcgaaaggggtctgatcaagtacgccagcaacgagaaggagcggaatatcgtgtcccggttcaagggctttgccacatacttcaccggcttcaacacaaaccgcctgaacatgtacagcgaggaagccaaatctacggccattagcttccggctgatcaaccagaacctcatcaaattcatcgacaatatcctggtgtacaagaaggtgtctcagaccctcccttctgatgtcctgagcaacatctacatcgacttcaaggccatcatcaataccagcagcctggaggagttcttctccatcaacaactacaacaacatcctgacccagaagcagatcgagatcttcaacgctgtgatcggcggaaagaaggataaggatgagaaaattatcacaaagggcttcaaccagtacatcaatgaatataatcagaccaacaagaatatcagactgccaaagatgatgagactgttcaatcagatactgagcgaccgggaaggcgtgtcagctagacctgagcccttcaacaacgccaacgagacaatcagctccgtgagagactgttttacaaacgaaatcagcaagcagatcaccatcctgtctgaaaccaccagtaagatcgagagcttcgacatcgatagaatctacatcaagggcggagaggacctgcgggccctgagcaacagcatctacggctacttcaactacatccacgatagaatcgctgataagtggaagcacaacaatcctcagggcaagaagagccccgagagctaccaaaagaatctgaacgcctacctgaagggcataaagagcgtgagcctgcattctatcgccaacatctgtggcgacaacaaggtgatcgaatattttagaaatctcggcgccgagaacacagtggattttcagagagaaaacgtggtgtccctaattgacaacaaatacaactgtgcctcaaacctgctgtccgacgcccaaatcaccgacgaggagctgaggaccaacagcagaagcatcaaggatctgctcgacgccgtgaagagtgcccagagattcttcagactgctgtgcggttctggcaatgagcctgataaagaccacagcttttatgacgagtacacccctgctttcgaggccctggaaaacagcatcaaccccctgtacaacaaggtccgcagcttcgtgaccaaaaaggacttcagcacagacaagttcaaactgaacttcgacagcagcagcttcctgagcggatgggccaagaaaagcgagtacgagaagagcagcgctttcatcttcatcagggataatcagtactacctgggaattaataagtgcctgagtaaagaggacatcgcctacctggaggacagcacctctagcagcgacacaaagagagtggtgtacatgtttcagaaggtggatgccaccaatatcccaagaatcttcatcagatccaagggcagcaacctggcccctgctgtgaacgagttccagctgcctatcgaaaccatcctggatatctacgacaacaagttcttcaccaccagttaccagaagaaggatagaaccaaatggaaggaaagcctgaccaagctgatcgactactacaagctgggctttagccagcacaagtcctatgccgatttcgatttaaagtggaaagccagctcagaatacaatgacatcaatgatttcctggccgacgtgcagagattctgctacagaattgagttcatcaatatcaattgggacaagctcatcgagttcacagaggacggcaagttctacctgtttagaatcgccaacaaagacctgtctggcaacagcactggcctgcccaatctgcacaccatctactggaagatgctgttcgacgagagcaacctgaaggacatcgtgtacaagctgagcggcaacgctgaggtgtttatgcgctacaacagcctgaagaaccccattgtgcacaaggccggagtggaaatcaagaataagtgtcctttcaccgagaagaaaaccagcatctttgactacgacattatcaaggaccgcagatacaccaaggaccagctggaactgcatgtgcctatcctgatgaacttcaagtctccatctgccgctaaaggcaaagcctttaacaaggagtgcctggaatacatcagaaacaacggcatcaagcacatcatcggcatcgacagaggagagcggaatctgctttacatggtgatcacagacctggacggcaacatcgtggaacagaagtctctgaaccagatcgcctccaatccaaagctgcctctgttcagacaggactacaacaagctgctgaaaaccaaagctgacgccaacgcacaagccagaagagactgggagacaatagacaccgtgaaggagattaagttcggcttcctgagccagatcgtgcacgagatcgctatggccatcatcaagtacgacgccattgtggtcctggaaaacctgaacagaggcttcatgcaaaaacggggcctggaaaacaacgtgtatcagaagttcgagcaaatgctcctcgataaactgagctactatgtcgacaagaccaaacaccctgaggaagctggcggagccctgcacgcctatcagttaagcgatacctacgccaacttcaattccttgagcaagaacgctatggtgagacagtctggcttcgtgttctacatccccgcctggctgaccagcaagatcgatcctgtgaccggcttcgcctctttcctgaagttccacagagatgatagcatggccaccatcaagagcaccatctccaaattcgactgcttcaagtacgacaaggaatgcgacatgttccacatcagaatagattacaacaaatttagcacttcatgcagcggtggccagcggaagtgggatctgttcacattcggagacagaatcctggccgagagaaacaccatgcagaacagtagatacgtttaccagacagttaacctgacctctgagttcaagaacctgttcgccacaaaggatatcgatataagcgggaacctgaaggatagcatctgcaagatcgaggacgtgggcttcttccggaagctgagccagctgctgagcctgacactacagcttcggaacagcaacgctgaaaccggagaagatttcctgatcagccctgtggccgacaaggacggcaacttctttgacagcagaaactgccccgacagcctgccaaaggatgcagacgcgaatggcgcttataacattgccaggaagggcctgatgctggtggagcaactgaagcggtgcaaggacgtgagcaagttcaagcctgctatcaagaacgaggactggctggactacgtgcagcggtctagaaagcggacagcagacggctccgaatttgaaagccctaagaaaaagagaaaggtgggatccggcaaacctatccccaatcccctgctgggcctggacagcacctga

SEQ ID NO: 25 - protein, 1214 aa, unidentified
KINAFINCYSMSKTLRFKLAPEYETEKNLLEKGFLDRDKLRADDYDLMKKVIDKYHKHFIDKALEGFKFDLLQEYAEAFYSQSADDDGKKLEEIKKKMCKELATCFSKQDEFKLLDKKELVEKLIPAAEFIEDEEKDIAKRFKGFTTYFTGFNENRQNLYAAELKHGTIAFRLIEENLPAFLYNCKKGVKIFEGLDAVDAETLNNELGEILSIENVKDVLSVEYYNKTLTQNGIDVYNRIIGGYTQEDGTKIKGVNEYVNLYNQTHDKKLPSLAKLKKQILSDSYSLSFLPAKFNDDSELLLSLKKFYSTVNEETGLSVEKAIQEMRDVFSHIDDCDLHNVFIDAKFINKVSNDVFGNWSVLIDGINAEYEKLNPFNGKNLDNYEEKRKAFLNKIESYSVDALQAYSGKEEKIADYVQKRAVELYDSVACAYENMSNKVINAREGKVKLYQDDEKTEIIKTFLDAVQEFKKFAEMFCYDGTDGDTTFYGEFANYYGQIAEIIPLYNKCRNYLTKKPYSEDKIKINFDNAELLHGWDANKEKNYLTVLLFKNGSYYLGILDKKHKNVLIKDVPEKTQEEPCFKKMIYKLLPDPKRNMPRIILHAKSNKKLFEPSDEIYRIYETESFKTDIDDCHRLIDFYKESISKYEDWKTFGFKFKETSEYKNIGQFYNEVKEQGYKISFTDIPESYVKDLVNDGKLYLFRLANKDFSPYSKGKKNLHTMYFEGIFDPENIKEKVYALNGGGELFFRCASLNYDKPTHPKNVPIKNKTYDFRTDNAKKETSTFEYDLIKDKRYTKDQYTLHCPVTLNFKERGIERINDLVRQSLRESDDNYVIGIDRGERNLIYISVIDGKGKIVEQFSMNNLLSGNDVSIDFHKMLETREHERDASRKNWNTIDNIKDLKQGYLSYVVKKICDLVVKYDAIVAMEDLNVGFKHGREKFERQVYQKFEKALVDKMSYIVNKNASPHSDGGLFRAYQLTNKKYNENEKQNGFIFYVRAWNTSKIDPTTGFVNMLPLKYQSKEKSKEFFDKFEDIFYDENKDMFGFTFRYDDFGINIDHKNEWTAYSNGERIITVRNSFGKWDKAKIVLTPAFKKLFDDYNVDCRGDVKRQIMNVDDKDFFVRLYKLLSYTMQLRNSDDVDDYILSPVVNAEGKFFDSRNSDGSLPCDADANGAYHIAKKAMWAIGKIKEADEESFKKTSLAIDNKTWLEFVQKA

SEQ ID NO: 26 - protein, 1215 aa, unidentified
MKINAFINCYSMSKTLRFKLAPEYETEKNLLEKGFLDRDKLRADDYDLMKKVIDKYHKHFIDKALEGFKFDLLQEYAEAFYSQSADDDGKKLEEIKKKMCKELATCFSKQDEFKLLDKKELVEKLIPAAEFIEDEEKDIAKRFKGFTTYFTGFNENRQNLYAAELKHGTIAFRLIEENLPAFLYNCKKGVKIFEGLDAVDAETLNNELGEILSIENVKDVLSVEYYNKTLTQNGIDVYNRIIGGYTQEDGTKIKGVNEYVNLYNQTHDKKLPSLAKLKKQILSDSYSLSFLPAKFNDDSELLLSLKKFYSTVNEETGLSVEKAIQEMRDVFSHIDDCDLHNVFIDAKFINKVSNDVFGNWSVLIDGINAEYEKLNPFNGKNLDNYEEKRKAFLNKIESYSVDALQAYSGKEEKIADYVQKRAVELYDSVACAYENMSNKVINAREGKVKLYQDDEKTEIIKTFLDAVQEFKKFAEMFCYDGTDGDTTFYGEFANYYGQIAEIIPLYNKCRNYLTKKPYSEDKIKINFDNAELLHGWDANKEKNYLTVLLFKNGSYYLGILDKKHKNVLIKDVPEKTQEEPCFKKMIYKLLPDPKRNMPRIILHAKSNKKLFEPSDEIYRIYETESFKTDIDDCHRLIDFYKESISKYEDWKTFGFKFKETSEYKNIGQFYNEVKEQGYKISFTDIPESYVKDLVNDGKLYLFRLANKDFSPYSKGKKNLHTMYFEGIFDPENIKEKVYALNGGGELFFRCASLNYDKPTHPKNVPIKNKTYDFRTDNAKKETSTFEYDLIKDKRYTKDQYTLHCPVTLNFKERGIERINDLVRQSLRESDDNYVIGIDRGERNLIYISVIDGKGKIVEQFSMNNLLSGNDVSIDFHKMLETREHERDASRKNWNTIDNIKDLKQGYLSYVVKKICDLVVKYDAIVAMEDLNVGFKHGREKFERQVYQKFEKALVDKMSYIVNKNASPHSDGGLFRAYQLTNKKYNENEKQNGFIFYVRAWNTSKIDPTTGFVNMLPLKYQSKEKSKEFFDKFEDIFYDENKDMFGFTFRYDDFGINIDHKNEWTAYSNGERIITVRNSFGKWDKAKIVLTPAFKKLFDDYNVDCRGDVKRQIMNVDDKDFFVRLYKLLSYTMQLRNSDDVDDYILSPVVNAEGKFFDSRNSDGSLPCDADANGAYHIAKKAMWAIGKIKEADEESFKKTSLAIDNKTWLEFVQKA

SEQ ID NO: 27 - protein, 1252 aa, synthetic construct
MGKINAFINCYSMSKTLRFKLAPEYETEKNLLEKGFLDRDKLRADDYDLMKKVIDKYHKHFIDKALEGFKFDLLQEYAEAFYSQSADDDGKKLEEIKKKMCKELATCFSKQDEFKLLDKKELVEKLIPAAEFIEDEEKDIAKRFKGFTTYFTGFNENRQNLYAAELKHGTIAFRLIEENLPAFLYNCKKGVKIFEGLDAVDAETLNNELGEILSIENVKDVLSVEYYNKTLTQNGIDVYNRIIGGYTQEDGTKIKGVNEYVNLYNQTHDKKLPSLAKLKKQILSDSYSLSFLPAKFNDDSELLLSLKKFYSTVNEETGLSVEKAIQEMRDVFSHIDDCDLHNVFIDAKFINKVSNDVFGNWSVLIDGINAEYEKLNPFNGKNLDNYEEKRKAFLNKIESYSVDALQAYSGKEEKIADYVQKRAVELYDSVACAYENMSNKVINAREGKVKLYQDDEKTEIIKTFLDAVQEFKKFAEMFCYDGTDGDTTFYGEFANYYGQIAEIIPLYNKCRNYLTKKPYSEDKIKINFDNAELLHGWDANKEKNYLTVLLFKNGSYYLGILDKKHKNVLIKDVPEKTQEEPCFKKMIYKLLPDPKRNMPRIILHAKSNKKLFEPSDEIYRIYETESFKTDIDDCHRLIDFYKESISKYEDWKTFGFKFKETSEYKNIGQFYNEVKEQGYKISFTDIPESYVKDLVNDGKLYLFRLANKDFSPYSKGKKNLHTMYFEGIFDPENIKEKVYALNGGGELFFRCASLNYDKPTHPKNVPIKNKTYDFRTDNAKKETSTFEYDLIKDKRYTKDQYTLHCPVTLNFKERGIERINDLVRQSLRESDDNYVIGIDRGERNLIYISVIDGKGKIVEQFSMNNLLSGNDVSIDFHKMLETREHERDASRKNWNTIDNIKDLKQGYLSYVVKKICDLVVKYDAIVAMEDLNVGFKHGREKFERQVYQKFEKALVDKMSYIVNKNASPHSDGGLFRAYQLTNKKYNENEKQNGFIFYVRAWNTSKIDPTTGFVNMLPLKYQSKEKSKEFFDKFEDIFYDENKDMFGFTFRYDDFGINIDHKNEWTAYSNGERIITVRNSFGKWDKAKIVLTPAFKKLFDDYNVDCRGDVKRQIMNVDDKDFFVRLYKLLSYTMQLRNSDDVDDYILSPVVNAEGKFFDSRNSDGSLPCDADANGAYHIAKKAMWAIGKIKEADEESFKKTSLAIDNKTWLEFVQKASRKRTADGSEFESPKKKRKVGSGKPIPNPLLGLDST

SEQ ID NO: 28 - DNA, 3648 bp, unidentified
  source 1..3648: mol_type = unassigned DNA
atgaaaattaacgcttttatcaactgttattcgatgtccaagacgttgcgattcaagcttgcgcccgaatacgagacggaaaagaaccttttggaaaagggatttcttgatcgcgacaaattgcgcgcggacgattatgatttaatgaaaaaagttatcgataaatatcacaaacattttatcgataaagcgttggaaggtttcaaattcgatttattgcaagagtatgccgaagcgttttattcgcaatcggccgatgacgacggcaaaaaacttgaagaaatcaaaaagaaaatgtgcaaggagttggcgacttgtttttcgaaacaagacgagtttaaattactcgataaaaaagaactggtcgaaaaactaatccctgctgccgaatttattgaagacgaagaaaaagatattgcgaagagattcaaggggtttacgacctatttcacgggattcaacgaaaacaggcaaaacttatacgccgcagaactgaaacacgggacgattgcgttcagattgattgaagaaaatttgcctgcatttttgtacaactgcaaaaagggagtaaaaatattcgagggactcgacgcagtcgatgcagaaacgcttaataatgaacttggagagattctttcaatcgaaaacgtaaaagatgtattaagcgtagagtattacaataaaacgctcacgcaaaacggcatagacgtttacaaccggattataggcggctatacacaggaagacgggacgaaaatcaaaggtgtcaacgagtacgtcaatttgtataaccagacgcacgacaaaaaacttccgtcgctcgcaaaactcaaaaaacagattttaagcgacagttattcgttgtcgtttttgcccgcaaaattcaacgacgattccgaattgcttttatcgcttaaaaagttttattcgacggtaaacgaagagaccggtttaagcgtagaaaaggcgatacaggaaatgcgcgacgttttttcacacatcgatgactgtgatttgcataacgtttttatcgacgcaaaatttataaacaaggtttcaaacgacgttttcgggaattggagcgttttgattgacggcataaatgcggaatatgagaaactcaatccgttcaacgggaaaaacctcgacaattatgaggaaaaacgcaaagcgtttttaaacaagatcgaaagctattctgttgacgcgttgcaggcatattcgggtaaagaagaaaaaatcgccgactacgttcaaaaacgtgcggtcgaactttacgatagtgtcgcatgcgcatatgagaatatgagtaataaggtaataaatgcgcgagaagggaaggttaaactttatcaggacgatgaaaaaaccgaaataatcaaaacgtttttggacgcggtacaggaattcaaaaagtttgccgagatgttttgctatgacggcaccgacggcgatacgacgttttacggcgaatttgcgaattattacggacaaattgccgaaattataccgctttacaataaatgcaggaactatttgacgaaaaagccgtattccgaagacaaaatcaaaataaactttgacaacgctgagcttttgcatggatgggacgcaaacaaagaaaagaattatctgactgtattattatttaaaaacggcagttattatctcggtattctggataaaaagcataagaacgttttgatcaaagacgtgcccgaaaagacgcaggaggagccgtgtttcaagaaaatgatttacaaattactccctgatccgaaacgaaatatgcctagaataatattacatgcaaaaagtaacaagaagttgtttgagcctagtgatgagatatataggatatatgaaacagaatcgtttaaaactgacattgacgactgccataggttgattgatttttataaagaaagtataagcaagtacgaggactggaagacgttcgggttcaagttcaaagaaacgagcgagtataaaaacatagggcaattttataacgaagttaaagagcagggatataagatttcattcacggatatacccgaaagttacgtcaaagacttggtaaacgacgggaaactgtatttattcaggcttgctaataaagatttttctccgtacagcaagggcaaaaagaatttgcatacgatgtatttcgagggaatatttgatcctgaaaacataaaagaaaaggtttatgcgcttaacggcggcggcgagttgtttttcagatgcgcgagcttgaattacgacaaaccgacgcatccgaaaaacgtaccgattaaaaacaaaacgtatgatttccgcaccgataatgcgaaaaaagaaacaagcacgtttgaatacgacctcataaaagataagcgatatacgaaagatcaatacacgttgcattgtccggtgacgcttaattttaaggaaagaggaatcgaaagaataaacgatctcgtaaggcaatcgttgcgtgaaagtgacgacaactacgtaatcggcattgatcggggcgaaagaaacttaatttacatcagtgttatcgacggaaaaggaaagattgtcgagcaattctcgatgaacaatttgttaagcggtaacgacgtgtcgatagatttccacaaaatgctcgaaacgcgggagcacgagcgcgacgcgtccagaaaaaactggaatacaatcgacaatatcaaagacttgaagcaaggatatttaagttatgtcgtaaagaaaatttgcgaccttgtcgtaaaatacgacgcgattgtcgcaatggaagacttaaacgtcgggttcaagcacggacgagaaaagttcgagcgacaggtatatcagaaatttgaaaaagcacttgtcgacaaaatgagttatatcgtaaacaagaacgcgtcgccgcattccgacggaggtttgttcagggcataccagctgaccaataaaaagtataatgaaaacgaaaaacaaaacggttttattttctatgtcagagcgtggaataccagtaagatcgatccgacgaccgggtttgtaaacatgcttccgttaaaatatcagagcaaagaaaaatcaaaagaatttttcgataaatttgaagatattttttacgatgaaaacaaggatatgttcggttttacattcagatatgacgatttcggtataaatatcgatcataaaaacgaatggacggcttattcaaacggcgaacgaataatcaccgtacgaaattcgttcggcaagtgggataaagcgaagatcgtattgacgccggcatttaagaaactgtttgacgactataacgtggattgtcgcggcgacgtcaaacgacagattatgaacgttgacgacaaagactttttcgttaggttatataagcttttgtcgtatacgatgcagttgagaaactccgacgatgttgacgactatattttgtcgcccgtcgttaatgcggaagggaagttctttgacagtcgcaattcggacggcagtttgccttgcgacgcggacgcaaacggagcgtatcatattgccaaaaaggcaatgtgggcaatcgggaagataaaagaagcggacgaagaaagttttaaaaagacaagtcttgcaatcgacaacaagacgtggcttgaattcgttcaaaaggcataa

SEQ ID NO: 29 - DNA, 3642 bp, synthetic construct
  source 1..3642: mol_type = other DNA
aagatcaacgcttttatcaactgttacagcatgagcaagaccctgagattcaagctggcccctgagtacgaaaccgagaagaacctgctggaaaagggctttctggaccgggacaagctgagagccgacgactacgacctgatgaagaaggtgatagacaagtaccacaagcacttcatcgacaaggccctggaaggcttcaagtttgacctgctgcaagaatacgctgaggccttttacagccagagcgccgacgacgacggcaagaagctcgaagagatcaagaagaagatgtgcaaggagctggccacatgcttcagcaagcaagacgagttcaagctactggataagaaagagctggtggaaaagctgatcccagccgctgagttcatcgaggacgaggaaaaagacattgccaagagattcaaaggctttacaacctactttaccggcttcaatgaaaacagacagaatctgtacgccgccgagctgaagcacggaacaatcgccttcagactgatcgaggagaacttgcctgccttcctgtacaattgcaagaagggtgttaagatcttcgagggcctggacgctgtggatgctgagactctcaacaacgagctgggcgagatcctgagcatcgaaaacgtgaaggacgtgctgtccgtggagtactacaacaaaaccctgacccaaaacggcatcgatgtgtacaatagaatcatcggcggctacacccaggaggatggcaccaagatcaagggagtgaacgagtacgtgaacctgtataaccagacacacgacaagaaactgccttctctggctaagctgaagaagcaaatcctgtctgactcctattctctgtcattcctgcccgccaagtttaacgacgactctgagctcctgctcagcctgaagaagttttacagcaccgtgaacgaggaaacaggactgagcgtggagaaagctatccaggagatgagagatgtgttcagccacattgacgactgcgaccttcacaacgtctttatcgatgccaagttcatcaacaaggtgagcaacgacgtgttcggcaactggtcggtcctgatcgatggcatcaatgccgagtacgagaagctgaaccccttcaacggcaagaacctggacaactacgaggaaaaaagaaaggcctttctgaacaaaatcgagagctatagcgtggacgccctgcaggcctacagcggcaaggaagagaagatcgccgattatgtgcagaaacgggccgttgaactgtacgacagcgtggcttgtgcttacgaaaacatgagcaacaaagtgatcaacgcccgggaaggcaaggtgaagctgtaccaggacgacgaaaagaccgagattatcaagaccttcctggatgctgttcaggagttcaagaagttcgccgaaatgttctgctacgatggaacagatggagataccaccttctacggcgagttcgccaattattacggccagatcgccgagataatccccctgtacaacaagtgcagaaactatctgacaaagaaaccttacagcgaggacaagattaagatcaacttcgataacgcggaactgctgcatggatgggacgccaacaaggaaaagaactacctgacagtcctgctgttcaaaaatggatcatattacctgggcatcctggataaaaagcataagaacgtgctgattaaggacgttcctgaaaagacacaggaagagccctgtttcaaaaaaatgatctacaagctgctgcctgatcccaagcggaatatgcctaggatcatcttgcacgccaaaagcaataaaaaactgttcgagcctagcgatgagatctacagaatctatgagacagagagcttcaagaccgacatcgacgattgccacagactgatcgatttctacaaggaatccatcagcaagtacgaggactggaaaacctttggatttaaattcaaagaaaccagcgagtacaagaacatcggacagttctacaacgaggtgaaggaacagggctacaagattagcttcaccgacatccctgagagctacgtgaaggatctggtgaatgatggcaagctgtatctgtttagactcgccaacaaggatttctctccatactccaagggcaaaaagaacctgcacaccatgtacttcgagggaatcttcgaccccgaaaacatcaaggagaaagtgtacgccctgaacggcggcggcgagctgttcttccgctgtgcctctctgaactacgacaagcctacccaccccaagaacgtgcctatcaagaacaagacctacgattttagaaccgataacgctaagaaagaaaccagtacattcgagtacgacctgatcaaagataaacggtacacaaaggaccagtacacactgcactgccctgtgacactgaatttcaaggagcgtggaatcgaacgcatcaacgacctggtgcggcagagcctgcgggaaagcgacgacaactacgtcatcggcatcgacagaggggagagaaatctgatctacatctctgtgatcgacggcaagggcaagatcgtcgagcagttcagcatgaacaacctgctgtccggcaacgacgtcagcatcgacttccacaagatgctggaaaccagagagcacgagcgggacgcctccagaaagaactggaacaccatcgacaacatcaaggacctgaagcagggctacctgagttacgtggtgaaaaagatctgcgacctggtcgtgaagtatgatgccatcgtggctatggaggatctgaacgtgggctttaaacacggcagagagaagttcgagagacaggtgtaccagaagtttgagaaagccctggtggacaagatgagctacatcgtgaataaaaatgctagtcctcacagcgatggcggcctgttcagagcttatcagctgaccaacaagaaatacaacgagaatgaaaagcagaacggattcatcttttacgtgagagcctggaataccagcaagatcgacccaacaacaggcttcgtgaacatgttgccactgaaataccaatctaaggaaaagtccaaggagttcttcgacaagttcgaggatatcttctatgatgaaaacaaagacatgttcggcttcaccttccggtacgacgacttcggcatcaacatcgaccacaagaatgaatggaccgcctacagcaatggtgagcggatcatcaccgtgcggaacagcttcggcaaatgggataaagcgaagatcgtgctgacccctgcttttaagaagctgttcgatgattacaacgtggactgcagaggcgacgtgaagcgacagattatgaacgtggacgacaaagatttcttcgtgcggctgtacaagctgctgagctacaccatgcagctgagaaacagcgacgacgtggacgattacatcctgagccccgtggtgaatgccgaaggcaagttcttcgacagcagaaactctgacggctctctgccttgtgacgccgatgccaacggcgcctaccacatcgccaagaaggccatgtgggccatcggcaagatcaaggaagccgatgaggaatcttttaagaaaacctccctcgccatcgacaacaaaacctggctggagttcgtgcagaaagcc

SEQ ID NO: 30 - DNA, 3759 bp, synthetic construct
  source 1..3759: mol_type = other DNA
atgggcaagatcaacgcttttatcaactgttacagcatgagcaagaccctgagattcaagctggcccctgagtacgaaaccgagaagaacctgctggaaaagggctttctggaccgggacaagctgagagccgacgactacgacctgatgaagaaggtgatagacaagtaccacaagcacttcatcgacaaggccctggaaggcttcaagtttgacctgctgcaagaatacgctgaggccttttacagccagagcgccgacgacgacggcaagaagctcgaagagatcaagaagaagatgtgcaaggagctggccacatgcttcagcaagcaagacgagttcaagctactggataagaaagagctggtggaaaagctgatcccagccgctgagttcatcgaggacgaggaaaaagacattgccaagagattcaaaggctttacaacctactttaccggcttcaatgaaaacagacagaatctgtacgccgccgagctgaagcacggaacaatcgccttcagactgatcgaggagaacttgcctgccttcctgtacaattgcaagaagggtgttaagatcttcgagggcctggacgctgtggatgctgagactctcaacaacgagctgggcgagatcctgagcatcgaaaacgtgaaggacgtgctgtccgtggagtactacaacaaaaccctgacccaaaacggcatcgatgtgtacaatagaatcatcggcggctacacccaggaggatggcaccaagatcaagggagtgaacgagtacgtgaacctgtataaccagacacacgacaagaaactgccttctctggctaagctgaagaagcaaatcctgtctgactcctattctctgtcattcctgcccgccaagtttaacgacgactctgagctcctgctcagcctgaagaagttttacagcaccgtgaacgaggaaacaggactgagcgtggagaaagctatccaggagatgagagatgtgttcagccacattgacgactgcgaccttcacaacgtctttatcgatgccaagttcatcaacaaggtgagcaacgacgtgttcggcaactggtcggtcctgatcgatggcatcaatgccgagtacgagaagctgaaccccttcaacggcaagaacctggacaactacgaggaaaaaagaaaggcctttctgaacaaaatcgagagctatagcgtggacgccctgcaggcctacagcggcaaggaagagaagatcgccgattatgtgcagaaacgggccgttgaactgtacgacagcgtggcttgtgcttacgaaaacatgagcaacaaagtgatcaacgcccgggaaggcaaggtgaagctgtaccaggacgacgaaaagaccgagattatcaagaccttcctggatgctgttcaggagttcaagaagttcgccgaaatgttctgctacgatggaacagatggagataccaccttctacggcgagttcgccaattattacggccagatcgccgagataatccccctgtacaacaagtgcagaaactatctgacaaagaaaccttacagcgaggacaagattaagatcaacttcgataacgcggaactgctgcatggatgggacgccaacaaggaaaagaactacctgacagtcctgctgttcaaaaatggatcatattacctgggcatcctggataaaaagcataagaacgtgctgattaaggacgttcctgaaaagacacaggaagagccctgtttcaaaaaaatgatctacaagctgctgcctgatcccaagcggaatatgcctaggatcatcttgcacgccaaaagcaataaaaaactgttcgagcctagcgatgagatctacagaatctatgagacagagagcttcaagaccgacatcgacgattgccacagactgatcgatttctacaaggaatccatcagcaagtacgaggactggaaaacctttggatttaaattcaaagaaaccagcgagtacaagaacatcggacagttctacaacgaggtgaaggaacagggctacaagattagcttcaccgacatccctgagagctacgtgaaggatctggtgaatgatggcaagctgtatctgtttagactcgccaacaaggatttctctccatactccaagggcaaaaagaacctgcacaccatgtacttcgagggaatcttcgaccccgaaaacatcaaggagaaagtgtacgccctgaacggcggcggcgagctgttcttccgctgtgcctctctgaactacgacaagcctacccaccccaagaacgtgcctatcaagaacaagacctacgattttagaaccgataacgctaagaaagaaaccagtacattcgagtacgacctgatcaaagataaacggtacacaaaggaccagtacacactgcactgccctgtgacactgaatttcaaggagcgtggaatcgaacgcatcaacgacctggtgcggcagagcctgcgggaaagcgacgacaactacgtcatcggcatcgacagaggggagagaaatctgatctacatctctgtgatcgacggcaagggcaagatcgtcgagcagttcagcatgaacaacctgctgtccggcaacgacgtcagcatcgacttccacaagatgctggaaaccagagagcacgagcgggacgcctccagaaagaactggaacaccatcgacaacatcaaggacctgaagcagggctacctgagttacgtggtgaaaaagatctgcgacctggtcgtgaagtatgatgccatcgtggctatggaggatctgaacgtgggctttaaacacggcagagagaagttcgagagacaggtgtaccagaagtttgagaaagccctggtggacaagatgagctacatcgtgaataaaaatgctagtcctcacagcgatggcggcctgttcagagcttatcagctgaccaacaagaaatacaacgagaatgaaaagcagaacggattcatcttttacgtgagagcctggaataccagcaagatcgacccaacaacaggcttcgtgaacatgttgccactgaaataccaatctaaggaaaagtccaaggagttcttcgacaagttcgaggatatcttctatgatgaaaacaaagacatgttcggcttcaccttccggtacgacgacttcggcatcaacatcgaccacaagaatgaatggaccgcctacagcaatggtgagcggatcatcaccgtgcggaacagcttcggcaaatgggataaagcgaagatcgtgctgacccctgcttttaagaagctgttcgatgattacaacgtggactgcagaggcgacgtgaagcgacagattatgaacgtggacgacaaagatttcttcgtgcggctgtacaagctgctgagctacaccatgcagctgagaaacagcgacgacgtggacgattacatcctgagccccgtggtgaatgccgaaggcaagttcttcgacagcagaaactctgacggctctctgccttgtgacgccgatgccaacggcgcctaccacatcgccaagaaggccatgtgggccatcggcaagatcaaggaagccgatgaggaatcttttaagaaaacctccctcgccatcgacaacaaaacctggctggagttcgtgcagaaagcctctagaaagcggacagcagacggctccgaatttgaaagccctaagaaaaagagaaaggtgggatccggcaaacctatccccaatcccctgctgggcctggacagcacctga

SEQ ID NO: 31 - protein, 1301 aa, unidentified
SKLSTFNEHFQKTLTLRNELVPVGKTLENIISSNVLINDEKRSEDYKKAKEIIDSYHREFIEKSLSSVNVDWNDLYSYLSKKEPEDYAQKQKFLEELENILLEKRKIIVKQFEQYVFGSYTDSKGKKTKDLKFENLFKSELFDYLLPNFLKNDEDKKVIGSFNKFTSYFTGFYENRKNLYKSEPLPTAVAYRIVNENFPKFISNKNIFRVWKDNVPQFIEIAKTKLREEGISDLNIELKFDLTNFNSCLNQTGIDTYNDLIGQLNFAINLECQKDKNLCDLLRKKRSLKMVPLYKQILSDNDSSFSIDEFDNDESAIKDVISFYKKMIGENCPQRTLSELLHGLSSHDLEKIFVQGKNLNSVSKNLFGGKNWSLLRDAVIEEKSKEKVFKKVIKSNSTADELDKVLSKEEFSISFLSKVSGKDLSVEIDKFVKKQDELLVENNIQNWPSSLKNSEEKNLIKAPLDFLLNFYRFAQSFSSNNIDKDMSFYADFDESLSSLENVIGLYNKVRNYATKKPYTLEKIKLNFENPNLASGWSESKENDCLSIILLKEKKYFLGIFNKNNKPNFSEGISHSLSSNGCYRKMRYLLFKGFNKMLPKCAFTGEVKDHFKESSDDFSLFNKDTFISPLVITKEIFDLACSKEKVKKYQKEYEKINRAEYRQSLVKWITFGLKFLSSYKTTTQFDLSNLKRPEEYCDLKEFYEDVDNLTYKIEFLNIKEEDVDALVEKGQLYLFEIRNKDFAKNASGTPNLHTLYFKSIFDSKNLENGIVKLNGEAEIFYRKKSLKKDDITVHREGSYLVNKVCVDPNSGKTEQIPDKIYENIYAFVNGKSRDLSKEDEVYYAKATIKKATHEIVKDRRFTVDKFFFHCPITINYKSKDKPSKFNDKVLDFLRNNKDINIIGIDRGERNLIYVTVINQNGEIIDCKSFNTIKHQSSTVNYDVDYHNKLQEREKNRKEEKRSWNSITKIADLKEGYLSAVIHEVSLMMVKYNAIVVMENLNQGFKRIRGGIAERSVYQKFEKMLIDKLNYFVIKNENWTNPGGVLNGYQLTNKVSTIKDIGNQCGFLFYVPATYTSKIDPSTGFVNLINFNKYKNSEDRRKLICSFDKICFVQNENLFKFSIDYGKLCPDSKIAIKKWDVFSYGTRIIKENLTTGHIEENPEYDPTEELKSLLSSRGIEYQKGQNLLETIPTSDMTREFWNSLFKIFKAILQMRNSLTNSPIDRLLSPVKGKDGTFFDTDKVEGTKFEKLKDADANGAYNIALKGLLVLEKNDSVESNKDLKNVKKISLEDWLKFVQITLRD

SEQ ID NO: 32 - protein, 1302 aa, unidentified
MSKLSTFNEHFQKTLTLRNELVPVGKTLENIISSNVLINDEKRSEDYKKAKEIIDSYHREFIEKSLSSVNVDWNDLYSYLSKKEPEDYAQKQKFLEELENILLEKRKIIVKQFEQYVFGSYTDSKGKKTKDLKFENLFKSELFDYLLPNFLKNDEDKKVIGSFNKFTSYFTGFYENRKNLYKSEPLPTAVAYRIVNENFPKFISNKNIFRVWKDNVPQFIEIAKTKLREEGISDLNIELKFDLTNFNSCLNQTGIDTYNDLIGQLNFAINLECQKDKNLCDLLRKKRSLKMVPLYKQILSDNDSSFSIDEFDNDESAIKDVISFYKKMIGENCPQRTLSELLHGLSSHDLEKIFVQGKNLNSVSKNLFGGKNWSLLRDAVIEEKSKEKVFKKVIKSNSTADELDKVLSKEEFSISFLSKVSGKDLSVEIDKFVKKQDELLVENNIQNWPSSLKNSEEKNLIKAPLDFLLNFYRFAQSFSSNNIDKDMSFYADFDESLSSLENVIGLYNKVRNYATKKPYTLEKIKLNFENPNLASGWSESKENDCLSIILLKEKKYFLGIFNKNNKPNFSEGISHSLSSNGCYRKMRYLLFKGFNKMLPKCAFTGEVKDHFKESSDDFSLFNKDTFISPLVITKEIFDLACSKEKVKKYQKEYEKINRAEYRQSLVKWITFGLKFLSSYKTTTQFDLSNLKRPEEYCDLKEFYEDVDNLTYKIEFLNIKEEDVDALVEKGQLYLFEIRNKDFAKNASGTPNLHTLYFKSIFDSKNLENGIVKLNGEAEIFYRKKSLKKDDITVHREGSYLVNKVCVDPNSGKTEQIPDKIYENIYAFVNGKSRDLSKEDEVYYAKATIKKATHEIVKDRRFTVDKFFFHCPITINYKSKDKPSKFNDKVLDFLRNNKDINIIGIDRGERNLIYVTVINQNGEIIDCKSFNTIKHQSSTVNYDVDYHNKLQEREKNRKEEKRSWNSITKIADLKEGYLSAVIHEVSLMMVKYNAIVVMENLNQGFKRIRGGIAERSVYQKFEKMLIDKLNYFVIKNENWTNPGGVLNGYQLTNKVSTIKDIGNQCGFLFYVPATYTSKIDPSTGFVNLINFNKYKNSEDRRKLICSFDKICFVQNENLFKFSIDYGKLCPDSKIAIKKWDVFSYGTRIIKENLTTGHIEENPEYDPTEELKSLLSSRGIEYQKGQNLLETIPTSDMTREFWNSLFKIFKAILQMRNSLTNSPIDRLLSPVKGKDGTFFDTDKVEGTKFEKLKDADANGAYNIALKGLLVLEKNDSVESNKDLKNVKKISLEDWLKFVQITLRD

SEQ ID NO: 33 - protein, 1339 aa, synthetic construct
MGSKLSTFNEHFQKTLTLRNELVPVGKTLENIISSNVLINDEKRSEDYKKAKEIIDSYHREFIEKSLSSVNVDWNDLYSYLSKKEPEDYAQKQKFLEELENILLEKRKIIVKQFEQYVFGSYTDSKGKKTKDLKFENLFKSELFDYLLPNFLKNDEDKKVIGSFNKFTSYFTGFYENRKNLYKSEPLPTAVAYRIVNENFPKFISNKNIFRVWKDNVPQFIEIAKTKLREEGISDLNIELKFDLTNFNSCLNQTGIDTYNDLIGQLNFAINLECQKDKNLCDLLRKKRSLKMVPLYKQILSDNDSSFSIDEFDNDESAIKDVISFYKKMIGENCPQRTLSELLHGLSSHDLEKIFVQGKNLNSVSKNLFGGKNWSLLRDAVIEEKSKEKVFKKVIKSNSTADELDKVLSKEEFSISFLSKVSGKDLSVEIDKFVKKQDELLVENNIQNWPSSLKNSEEKNLIKAPLDFLLNFYRFAQSFSSNNIDKDMSFYADFDESLSSLENVIGLYNKVRNYATKKPYTLEKIKLNFENPNLASGWSESKENDCLSIILLKEKKYFLGIFNKNNKPNFSEGISHSLSSNGCYRKMRYLLFKGFNKMLPKCAFTGEVKDHFKESSDDFSLFNKDTFISPLVITKEIFDLACSKEKVKKYQKEYEKINRAEYRQSLVKWITFGLKFLSSYKTTTQFDLSNLKRPEEYCDLKEFYEDVDNLTYKIEFLNIKEEDVDALVEKGQLYLFEIRNKDFAKNASGTPNLHTLYFKSIFDSKNLENGIVKLNGEAEIFYRKKSLKKDDITVHREGSYLVNKVCVDPNSGKTEQIPDKIYENIYAFVNGKSRDLSKEDEVYYAKATIKKATHEIVKDRRFTVDKFFFHCPITINYKSKDKPSKFNDKVLDFLRNNKDINIIGIDRGERNLIYVTVINQNGEIIDCKSFNTIKHQSSTVNYDVDYHNKLQEREKNRKEEKRSWNSITKIADLKEGYLSAVIHEVSLMMVKYNAIVVMENLNQGFKRIRGGIAERSVYQKFEKMLIDKLNYFVIKNENWTNPGGVLNGYQLTNKVSTIKDIGNQCGFLFYVPATYTSKIDPSTGFVNLINFNKYKNSEDRRKLICSFDKICFVQNENLFKFSIDYGKLCPDSKIAIKKWDVFSYGTRIIKENLTTGHIEENPEYDPTEELKSLLSSRGIEYQKGQNLLETIPTSDMTREFWNSLFKIFKAILQMRNSLTNSPIDRLLSPVKGKDGTFFDTDKVEGTKFEKLKDADANGAYNIALKGLLVLEKNDSVESNKDLKNVKKISLEDWLKFVQITLRDSRKRTADGSEFESPKKKRKVGSGKPIPNPLLGLDST

SEQ ID NO: 34 - DNA, 3909 bp, unidentified
  source 1..3909: mol_type = unassigned DNA
atgtctaaattatcaacttttaatgaacattttcaaaaaacgttaactttaagaaacgaactagttcctgtaggaaaaactcttgaaaatatcatatcttcaaatgtattgataaatgatgagaaaagaagtgaagattataaaaaggctaaagagatcatagattcttatcatcgagagtttatagagaaatcactttcatcagtaaatgttgattggaatgatctgtactcgtatttatccaaaaaagaaccagaagactatgctcaaaagcagaagttcctcgaagagttagaaaatattctccttgaaaagagaaaaattattgttaaacagtttgagcaatacgttttcggatcatatacagattcaaaaggtaaaaaaacaaaagatctaaaatttgagaatctttttaaatcagagttgtttgattatcttttgccaaatttcctaaaaaatgatgaagataaaaaagtaataggtagttttaataaatttacatcgtattttacaggtttttacgaaaatcgaaagaatttatataaatcagagccattgccaacagctgtggcttatagaatagttaacgaaaactttcctaaattcatttctaataaaaatatctttcgcgtgtggaaagataatgttcctcagtttatagaaatagcgaaaactaaactaagagaagaaggcatttctgatttaaatatagaattaaaatttgatttaactaatttcaattcatgcttaaatcaaactggaattgatacttacaatgacttgataggtcaactcaactttgcaattaaccttgaatgtcagaaagacaagaatttatgtgaccttttaaggaagaaaagaagccttaaaatggtacctctgtataaacagattttatctgataatgattcttcattcagtattgatgaatttgataatgatgaatcggcaataaaagatgtaatttctttttataagaaaatgattggtgaaaattgtcctcaacgaacactatctgaattgctacatggtttgtcatctcacgatcttgaaaagatatttgttcaaggtaaaaacttaaattcggtttctaaaaatttatttggagggaagaactggtctttactaagggatgcagttatagaagaaaagtcaaaagaaaaagtcttcaaaaaggttataaagtcaaattctaccgcagatgaattagacaaagttctttccaaggaagaattttcaatttcattcttatcaaaagtgagcggtaaagatttatcagtagaaattgataaatttgtaaaaaaacaagacgaactacttgttgaaaataatatacaaaattggccaagttctcttaagaacagcgaagagaaaaatctcataaaagctcctttagatttcttacttaatttttatagatttgcacaatcattctcttcaaataatattgataaggatatgtcattttatgctgactttgatgaatctctatcgtctttagaaaatgtaataggtctttataacaaagtcagaaactatgcaactaagaaaccttatacactcgaaaagatcaaattgaattttgaaaatccaaatttagcttctggatggagtgaaagcaaagaaaatgattgtttatcaattatcttattaaaagagaaaaaatattttttaggaattttcaacaaaaataataaacctaatttttctgaaggcatttctcattcactttcttcaaatggttgctacagaaaaatgaggtatttattattcaagggattcaataaaatgcttcctaaatgtgcttttacaggagaagttaaagatcattttaaagaatcatcggatgatttttctctttttaacaaggatacttttatctctcctcttgtaattaccaaagagatctttgatttagcatgtagtaaagaaaaggtaaaaaaatatcaaaaagaatatgaaaagatcaatcgtgctgaatatagacaatcattggttaagtggattacttttggtcttaaatttttgtcatcatataaaactacaactcaatttgatttatcaaatttaaaaagacctgaagaatactgcgatctaaaggaattttatgaagatgtagataatcttacatacaagatagaatttttaaatataaaagaagaagatgtagatgcattggttgaaaaaggtcaactgtatttatttgaaattcgaaataaagattttgcaaaaaatgcaagtggcactcctaatctacatactctctattttaaaagtattttcgattcgaaaaatttagagaatggcattgtcaagcttaatggtgaagcagagatattttatagaaagaaaagcttgaagaaagatgacataactgttcatcgagaaggcagttatcttgtaaataaggtgtgtgtcgatcctaattctggaaaaacagaacagattcctgacaaaatttatgaaaatatttatgctttcgtaaatggtaaatcaagagatttatctaaggaggatgaagtatattatgcaaaagccacaataaaaaaagctacccatgagatcgtaaaagatagacgctttactgtagataaattctttttccactgccctattactattaactataaatctaaagataaaccttcaaaattcaatgacaaggttttagatttcttaagaaataataaagacatcaacattataggcatagatcgaggagagagaaatcttatttatgtaactgtaattaatcaaaatggcgaaattattgattgcaaatcatttaatactatcaaacatcagtcttcaacagtgaattacgatgttgattatcacaacaaattacaagaaagagaaaaaaatagaaaagaagaaaagagatcttggaatagtattactaaaattgcagatctcaaagaaggctatctttctgctgtaattcatgaagtttcattaatgatggttaagtacaatgccattgtcgttatggaaaatttgaatcaaggttttaagagaattagaggaggaattgctgaaagatccgtataccaaaaatttgaaaagatgctgatagataaactgaattattttgttataaaaaatgaaaattggacaaatcctggtggggtcctcaatggatatcagttaactaacaaagtgtctacaatcaaagatatcggtaatcagtgtggatttttattttacgttcctgcaacttatacctcaaagattgatccttctacaggctttgttaatttaattaatttcaataaatataaaaattcagaagatcgaagaaaactcatttgtagctttgacaagatatgctttgtacagaatgagaatttatttaaattttctatagattatggaaaattatgcccagatagcaaaattgctataaaaaaatgggatgttttctcctacggaacaagaattattaaggaaaatctaacaactggtcatatagaagaaaatcctgaatacgatccgacagaagagcttaaatctctgctttcctcaagaggaattgagtatcaaaaaggtcaaaatttactagaaacaatacctactagtgatatgactagagaattttggaattctcttttcaagatttttaaagcaattttacaaatgagaaacagtctaactaattcaccaatagacaggcttttatctccagttaaaggaaaagatggaaccttctttgatacagataaagtagaaggtactaagtttgaaaagttaaaagacgctgatgcaaacggagcatataacattgcgttaaaaggattgttagtcctcgagaaaaatgattctgtagagtccaataaggatctaaaaaatgttaagaaaattagtcttgaggattggttaaagtttgtccaaatcacattaagagattaa

SEQ ID NO: 35 - DNA, 3903 bp, synthetic construct
  source 1..3903: mol_type = other DNA
agcaaattgtcgaccttcaatgagcactttcagaaaaccctgaccctgcggaatgagctggtgcccgtgggcaagacactggagaacatcatcagctctaacgtgctgatcaacgacgagaagcggtccgaggactacaaaaaggccaaggaaatcattgacagctatcaccgggagttcatcgagaaaagcctgagctctgtgaatgtggactggaatgatctgtacagctacctgagcaagaaagaacccgaggactatgcccagaaacagaagttcctggaggagttagagaacatcctgctggaaaagagaaagatcatcgtgaagcagttcgagcagtacgtgttcggttcctataccgacagcaagggaaaaaagaccaaggacctgaaattcgaaaacctgtttaagtccgaactctttgactacctgctgcctaacttcttgaaaaacgacgaggataagaaggtgattggctccttcaataagttcaccagctatttcaccggcttttacgagaacagaaaaaacctgtacaagagcgagcctctgcctaccgccgtcgcctacagaatcgtgaacgagaacttccccaagtttatctctaacaagaacatctttagagtgtggaaggacaacgtccctcaattcatcgagatcgcaaagaccaaactgagagaagaaggcatctctgatctgaacatcgagctgaagtttgatttgacaaatttcaactcctgcctgaatcagaccggcatcgatacctacaacgacctgatcggccagctgaactttgctatcaacctcgaatgtcagaaggacaagaacctttgtgacctgctgcgcaagaagcggagccttaagatggtgccactgtacaagcaaatcctgtccgacaacgatagcagcttcagcatcgacgagttcgacaatgatgaaagcgccatcaaggacgttatcagcttctacaagaagatgatcggcgagaactgccctcagcggaccctgtctgagctgctgcacggcctgtctagccacgatctggagaaaattttcgtgcaagggaagaacctgaacagcgtgtccaagaacctgttcggcggcaagaactggtccctgctgcgggacgccgtgatcgaggaaaaaagcaaagagaaggtgttcaagaaggtgatcaagagcaacagcaccgctgatgagctggataaggtgctgtctaaggaggagttcagcatctctttcctatccaaggtgtccggcaaggatctgagcgtggaaatcgacaagttcgtcaaaaaacaggacgagcttctggtggagaacaatatccagaactggccttcttctctcaagaatagcgaagaaaagaacctgatcaaggcccctctggactttttgttgaatttctacaggttcgcccagagcttcagcagcaacaacatcgataaagatatgtccttctacgctgattttgacgagtctctgtcaagcctggaaaatgtgataggcctgtacaacaaagtgcggaactacgccaccaagaaaccttacacactggaaaagatcaagctaaacttcgagaaccctaacctggcctctggatggagtgagagcaaggaaaacgattgcctgagtatcatcctgctgaaggagaagaaatacttcctgggcatcttcaacaagaacaacaagcccaacttttcagagggcatcagccacagcctgtcaagcaacggctgttaccggaagatgagatacctgctgttcaagggattcaacaagatgctgcctaagtgcgccttcacaggagaggtgaaggaccacttcaaggaaagctccgatgacttcagcctgttcaacaaggacaccttcatcagccccctggtgatcaccaaggaaattttcgatctggcttgcagcaaggaaaaagtgaagaagtaccaaaaagaatacgagaaaatcaacagagccgagtaccggcagtctctggtgaagtggatcacctttggcctgaagtttctgtctagctacaaaaccaccacccagttcgacctgagcaatttgaagcgccccgaggaatactgcgacctgaaagaattttacgaggacgtggataacttaacctacaagattgagttcctgaacattaaagaggaggacgtggacgctctggtcgagaaaggccagctgtacctgtttgagattagaaacaaggacttcgccaagaatgccagcggcacgcccaacctgcatacactgtatttcaagagcatcttcgatagcaagaacctggaaaatggcatcgtgaaactgaacggcgaggccgaaattttctacagaaagaagagcctgaagaaggatgatatcaccgtgcacagagagggaagctacctcgtcaacaaagtctgcgtggaccctaattccggcaagacagagcagatcccagataagatctacgagaacatctacgccttcgtcaacggcaagtcacgggacctgagcaaggaggacgaggtgtactacgccaaagccaccatcaagaaggctacccacgagatcgtgaaggatcgaagattcaccgtcgacaagttcttcttccactgccccatcactatcaactacaagagcaaagacaagccaagcaagtttaacgacaaagtgctggacttcctgagaaataacaaggacatcaatatcatcggcatcgacagaggcgaaagaaacttgatctacgtgaccgtgatcaaccagaacggagagatcatcgactgtaagagcttcaataccattaagcaccagagcagcacagtgaactacgacgtggactaccacaacaagctgcaggagcgggaaaagaacagaaaggaagaaaagagatcttggaacagcatcaccaagatcgccgatctgaaagagggctacctgtctgccgtgattcacgaggttagcctgatgatggtgaagtacaacgccatagttgtgatggaaaacctgaaccagggcttcaagagaatccggggcggcatcgccgaacggagcgtgtaccaaaagtttgaaaagatgctcatcgacaagctgaactacttcgtgatcaagaacgagaactggaccaatcctggcggagtgctgaatggataccagctgacaaacaaggtgtccacaatcaaggatattggaaatcagtgcggcttcctgttctacgtgcccgccacttatacatctaaaatcgatcctagcactggatttgtgaacctgatcaacttcaacaagtacaagaacagcgaggacagaaggaagctgatctgtagcttcgacaagatctgctttgtgcagaatgagaacctgttcaagttctctatcgattacggcaaactgtgccctgacagcaagatcgccatcaaaaagtgggacgtattctcctatggcaccaggatcatcaaggaaaacctgacaacaggccacatcgaagaaaatccagagtacgaccctacagaggaactgaaatccctgctttccagcagaggcatcgagtaccagaagggccaaaacctgctagaaaccatccctaccagcgacatgaccagagagttctggaatagcctgttcaagatcttcaaggccatcctgcagatgagaaactctctgacaaactctcctatcgaccggctgctaagccctgtgaaggggaaagatggaaccttcttcgacaccgacaaggtggaaggcacaaaatttgagaaactgaaggacgctgacgctaacggcgcctacaacatcgccctgaagggcctgctggtgctggaaaaaaacgactctgtcgagagcaacaaggacctcaagaacgtgaagaaaatctcactggaggactggctgaaattcgtgcagatcacacttagagac

SEQ ID NO: 36 - DNA, 4020 bp, synthetic construct
  source 1..4020: mol_type = other DNA
atgggcagcaaattgtcgaccttcaatgagcactttcagaaaaccctgaccctgcggaatgagctggtgcccgtgggcaagacactggagaacatcatcagctctaacgtgctgatcaacgacgagaagcggtccgaggactacaaaaaggccaaggaaatcattgacagctatcaccgggagttcatcgagaaaagcctgagctctgtgaatgtggactggaatgatctgtacagctacctgagcaagaaagaacccgaggactatgcccagaaacagaagttcctggaggagttagagaacatcctgctggaaaagagaaagatcatcgtgaagcagttcgagcagtacgtgttcggttcctataccgacagcaagggaaaaaagaccaaggacctgaaattcgaaaacctgtttaagtccgaactctttgactacctgctgcctaacttcttgaaaaacgacgaggataagaaggtgattggctccttcaataagttcaccagctatttcaccggcttttacgagaacagaaaaaacctgtacaagagcgagcctctgcctaccgccgtcgcctacagaatcgtgaacgagaacttccccaagtttatctctaacaagaacatctttagagtgtggaaggacaacgtccctcaattcatcgagatcgcaaagaccaaactgagagaagaaggcatctctgatctgaacatcgagctgaagtttgatttgacaaatttcaactcctgcctgaatcagaccggcatcgatacctacaacgacctgatcggccagctgaactttgctatcaacctcgaatgtcagaaggacaagaacctttgtgacctgctgcgcaagaagcggagccttaagatggtgccactgtacaagcaaatcctgtccgacaacgatagcagcttcagcatcgacgagttcgacaatgatgaaagcgccatcaaggacgttatcagcttctacaagaagatgatcggcgagaactgccctcagcggaccctgtctgagctgctgcacggcctgtctagccacgatctggagaaaattttcgtgcaagggaagaacctgaacagcgtgtccaagaacctgttcggcggcaagaactggtccctgctgcgggacgccgtgatcgaggaaaaaagcaaagagaaggtgttcaagaaggtgatcaagagcaacagcaccgctgatgagctggataaggtgctgtctaaggaggagttcagcatctctttcctatccaaggtgtccggcaaggatctgagcgtggaaatcgacaagttcgtcaaaaaacaggacgagcttctggtggagaacaatatccagaactggccttcttctctcaagaatagcgaagaaaagaacctgatcaaggcccctctggactttttgttgaatttctacaggttcgcccagagcttcagcagcaacaacatcgataaagatatgtccttctacgctgattttgacgagtctctgtcaagcctggaaaatgtgataggcctgtacaacaaagtgcggaactacgccaccaagaaaccttacacactggaaaagatcaagctaaacttcgagaaccctaacctggcctctggatggagtgagagcaaggaaaacgattgcctgagtatcatcctgctgaaggagaagaaatacttcctgggcatcttcaacaagaacaacaagcccaacttttcagagggcatcagccacagcctgtcaagcaacggctgttaccggaagatgagatacctgctgttcaagggattcaacaagatgctgcctaagtgcgccttcacaggagaggtgaaggaccacttcaaggaaagctccgatgacttcagcctgttcaacaaggacaccttcatcagccccctggtgatcaccaaggaaattttcgatctggcttgcagcaaggaaaaagtgaagaagtaccaaaaagaatacgagaaaatcaacagagccgagtaccggcagtctctggtgaagtggatcacctttggcctgaagtttctgtctagctacaaaaccaccacccagttcgacctgagcaatttgaagcgccccgaggaatactgcgacctgaaagaattttacgaggacgtggataacttaacctacaagattgagttcctgaacattaaagaggaggacgtggacgctctggtcgagaaaggccagctgtacctgtttgagattagaaacaaggacttcgccaagaatgccagcggcacgcccaacctgcatacactgtatttcaagagcatcttcgatagcaagaacctggaaaatggcatcgtgaaactgaacggcgaggccgaaattttctacagaaagaagagcctgaagaaggatgatatcaccgtgcacagagagggaagctacctcgtcaacaaagtctgcgtggaccctaattccggcaagacagagcagatcccagataagatctacgagaacatctacgccttcgtcaacggcaagtcacgggacctgagcaaggaggacgaggtgtactacgccaaagccaccatcaagaaggctacccacgagatcgtgaaggatcgaagattcaccgtcgacaagttcttcttccactgccccatcactatcaactacaagagcaaagacaagccaagcaagtttaacgacaaagtgctggacttcctgagaaataacaaggacatcaatatcatcggcatcgacagaggcgaaagaaacttgatctacgtgaccgtgatcaaccagaacggagagatcatcgactgtaagagcttcaataccattaagcaccagagcagcacagtgaactacgacgtggactaccacaacaagctgcaggagcgggaaaagaacagaaaggaagaaaagagatcttggaacagcatcaccaagatcgccgatctgaaagagggctacctgtctgccgtgattcacgaggttagcctgatgatggtgaagtacaacgccatagttgtgatggaaaacctgaaccagggcttcaagagaatccggggcggcatcgccgaacggagcgtgtaccaaaagtttgaaaagatgctcatcgacaagctgaactacttcgtgatcaagaacgagaactggaccaatcctggcggagtgctgaatggataccagctgacaaacaaggtgtccacaatcaaggatattggaaatcagtgcggcttcctgttctacgtgcccgccacttatacatctaaaatcgatcctagcactggatttgtgaacctgatcaacttcaacaagtacaagaacagcgaggacagaaggaagctgatctgtagcttcgacaagatctgctttgtgcagaatgagaacctgttcaagttctctatcgattacggcaaactgtgccctgacagcaagatcgccatcaaaaagtgggacgtattctcctatggcaccaggatcatcaaggaaaacctgacaacaggccacatcgaagaaaatccagagtacgaccctacagaggaactgaaatccctgctttccagcagaggcatcgagtaccagaagggccaaaacctgctagaaaccatccctaccagcgacatgaccagagagttctggaatagcctgttcaagatcttcaaggccatcctgcagatgagaaactctctgacaaactctcctatcgaccggctgctaagccctgtgaaggggaaagatggaaccttcttcgacaccgacaaggtggaaggcacaaaatttgagaaactgaaggacgctgacgctaacggcgcctacaacatcgccctgaagggcctgctggtgctggaaaaaaacgactctgtcgagagcaacaaggacctcaagaacgtgaagaaaatctcactggaggactggctgaaattcgtgcagatcacacttagagactctagaaagcggacagcagacggctccgaatttgaaagccctaagaaaaagagaaaggtgggatccggcaaacctatccccaatcccctgctgggcctggacagcacctga

SEQ ID NO: 37 - protein, 1270 aa, unidentified
KRLIDFTNIYQRSKTLRFRLEPIGKTADYIKNSQSLETDARLAKESKKVKELADEYHKEFIGDVLSSLELPLSKINELWDIYIYIYMSNDTDREIKFKKLQENLRKVIAEAFSKDKRFGNLFKKEIITDILPEFLQDKDDDIKIVNRFKGFTTYFYAFHKNRENMYVSEEKSTAIPYRIVNQNLVKYFDNYKTFKEKVMPLLKDKNIVESIERDFKDILNEKSIEDVFGLANFTHTLCQADIEKYNTLIGGLVVKNEKKEIKGINQYINEHNQTSKKGNGIPKLKPLFNQILSDRKSLSFTLDDIKKTSEAIRTIKDEYENLRDKLATIERLIKSIKEYDLAGIYIKMGEDTSTISQHWFGAYYKIIEAIADAWERRNPKKNRESKAYSKYVSSLKSISLQEIDDLKIGEPIENYFATFGTTCSDRTSGVSSLNRIKAAYTEFVNKFPEGFEDGDDCNDAYFKANVEVVKNLLDSIKDFQRFVKPLLGNEDERDKDEAFYGEFVPTYTDMDNIITPLYNRVRNFATKKPYSTDKIKINFENVVLLKGWDKNKESDYASIILMKDGQYFLGVLRNGSKSTLKTILPNTGDCYQKMVYKYFKDIKSNLPRCTTQRKDVKAHFAESSDDYTLLDTKAFVSALTISREVFELYNAPDKEKKFKKEYLKNTNDSIGYANAVSVCKRFCLEFLKKYRSTAIYDLSDVETSVDSFDDLSSFYQEIDKRLYSISFENVSVDSVNELVDNGNMLLFRIANKDFSPNSKGRPNLHTIYWRMLFDPANLKDVVYQLNGNAEIFFRKASVTRTEPTHPANVAIKNKSEYNKQNKPYSTFKYGLIKDRRYTTDQFEFHVPITMNFKQPESSKLQDKLNKQVLDFLKQDGVRHIIGIDRGERNLLYLVMVDMEGKIKKQISLNEIAGNPKNPEFKQDFLALLHEREGDRLESRRSWNTIQSIKELKEGYMSLVVHEIANMMLENDAIVVLENLNRSFMQKRGGIEKSVYQKFEKMLIDKLGYIVDKTKDVSDNGGALHAVQLADTFENFNKTQKGAIRQCGFIFYIPAWRTSKIDPVTGFVPMLRCQYESIVESKKFFGKFDSIYYDATGKYFVFQTDFTKFNTESKGGIQKWDICTYGDRIYAPRTKDRNNNPVSERVNLTEEMKSLFVSHNINIQGDIKAGIMQQTDKEFFESLHRLLRLTLQIRNSKKSTGKDYEDYIISPVMGKDGRFFDSRNADATQPKDADANGAYNIARKGLMLLRQIQAQEKQDLSNGKWLEFAQR

SEQ ID NO: 38 - protein, 1271 aa, unidentified
MKRLIDFTNIYQRSKTLRFRLEPIGKTADYIKNSQSLETDARLAKESKKVKELADEYHKEFIGDVLSSLELPLSKINELWDIYIYIYMSNDTDREIKFKKLQENLRKVIAEAFSKDKRFGNLFKKEIITDILPEFLQDKDDDIKIVNRFKGFTTYFYAFHKNRENMYVSEEKSTAIPYRIVNQNLVKYFDNYKTFKEKVMPLLKDKNIVESIERDFKDILNEKSIEDVFGLANFTHTLCQADIEKYNTLIGGLVVKNEKKEIKGINQYINEHNQTSKKGNGIPKLKPLFNQILSDRKSLSFTLDDIKKTSEAIRTIKDEYENLRDKLATIERLIKSIKEYDLAGIYIKMGEDTSTISQHWFGAYYKIIEAIADAWERRNPKKNRESKAYSKYVSSLKSISLQEIDDLKIGEPIENYFATFGTTCSDRTSGVSSLNRIKAAYTEFVNKFPEGFEDGDDCNDAYFKANVEVVKNLLDSIKDFQRFVKPLLGNEDERDKDEAFYGEFVPTYTDMDNIITPLYNRVRNFATKKPYSTDKIKINFENVVLLKGWDKNKESDYASIILMKDGQYFLGVLRNGSKSTLKTILPNTGDCYQKMVYKYFKDIKSNLPRCTTQRKDVKAHFAESSDDYTLLDTKAFVSALTISREVFELYNAPDKEKKFKKEYLKNTNDSIGYANAVSVCKRFCLEFLKKYRSTAIYDLSDVETSVDSFDDLSSFYQEIDKRLYSISFENVSVDSVNELVDNGNMLLFRIANKDFSPNSKGRPNLHTIYWRMLFDPANLKDVVYQLNGNAEIFFRKASVTRTEPTHPANVAIKNKSEYNKQNKPYSTFKYGLIKDRRYTTDQFEFHVPITMNFKQPESSKLQDKLNKQVLDFLKQDGVRHIIGIDRGERNLLYLVMVDMEGKIKKQISLNEIAGNPKNPEFKQDFLALLHEREGDRLESRRSWNTIQSIKELKEGYMSLVVHEIANMMLENDAIVVLENLNRSFMQKRGGIEKSVYQKFEKMLIDKLGYIVDKTKDVSDNGGALHAVQLADTFENFNKTQKGAIRQCGFIFYIPAWRTSKIDPVTGFVPMLRCQYESIVESKKFFGKFDSIYYDATGKYFVFQTDFTKFNTESKGGIQKWDICTYGDRIYAPRTKDRNNNPVSERVNLTEEMKSLFVSHNINIQGDIKAGIMQQTDKEFFESLHRLLRLTLQIRNSKKSTGKDYEDYIISPVMGKDGRFFDSRNADATQPKDADANGAYNIARKGLMLLRQIQAQEKQDLSNGKWLEFAQR

SEQ ID NO: 39 - protein, 1308 aa, synthetic construct
MGKRLIDFTNIYQRSKTLRFRLEPIGKTADYIKNSQSLETDARLAKESKKVKELADEYHKEFIGDVLSSLELPLSKINELWDIYIYIYMSNDTDREIKFKKLQENLRKVIAEAFSKDKRFGNLFKKEIITDILPEFLQDKDDDIKIVNRFKGFTTYFYAFHKNRENMYVSEEKSTAIPYRIVNQNLVKYFDNYKTFKEKVMPLLKDKNIVESIERDFKDILNEKSIEDVFGLANFTHTLCQADIEKYNTLIGGLVVKNEKKEIKGINQYINEHNQTSKKGNGIPKLKPLFNQILSDRKSLSFTLDDIKKTSEAIRTIKDEYENLRDKLATIERLIKSIKEYDLAGIYIKMGEDTSTISQHWFGAYYKIIEAIADAWERRNPKKNRESKAYSKYVSSLKSISLQEIDDLKIGEPIENYFATFGTTCSDRTSGVSSLNRIKAAYTEFVNKFPEGFEDGDDCNDAYFKANVEVVKNLLDSIKDFQRFVKPLLGNEDERDKDEAFYGEFVPTYTDMDNIITPLYNRVRNFATKKPYSTDKIKINFENVVLLKGWDKNKESDYASIILMKDGQYFLGVLRNGSKSTLKTILPNTGDCYQKMVYKYFKDIKSNLPRCTTQRKDVKAHFAESSDDYTLLDTKAFVSALTISREVFELYNAPDKEKKFKKEYLKNTNDSIGYANAVSVCKRFCLEFLKKYRSTAIYDLSDVETSVDSFDDLSSFYQEIDKRLYSISFENVSVDSVNELVDNGNMLLFRIANKDFSPNSKGRPNLHTIYWRMLFDPANLKDVVYQLNGNAEIFFRKASVTRTEPTHPANVAIKNKSEYNKQNKPYSTFKYGLIKDRRYTTDQFEFHVPITMNFKQPESSKLQDKLNKQVLDFLKQDGVRHIIGIDRGERNLLYLVMVDMEGKIKKQISLNEIAGNPKNPEFKQDFLALLHEREGDRLESRRSWNTIQSIKELKEGYMSLVVHEIANMMLENDAIVVLENLNRSFMQKRGGIEKSVYQKFEKMLIDKLGYIVDKTKDVSDNGGALHAVQLADTFENFNKTQKGAIRQCGFIFYIPAWRTSKIDPVTGFVPMLRCQYESIVESKKFFGKFDSIYYDATGKYFVFQTDFTKFNTESKGGIQKWDICTYGDRIYAPRTKDRNNNPVSERVNLTEEMKSLFVSHNINIQGDIKAGIMQQTDKEFFESLHRLLRLTLQIRNSKKSTGKDYEDYIISPVMGKDGRFFDSRNADATQPKDADANGAYNIARKGLMLLRQIQAQEKQDLSNGKWLEFAQRSRKRTADGSEFESPKKKRKVGSGKPIPNPLLGLDST

SEQ ID NO: 40 - DNA, 3816 bp, unidentified
  source 1..3816: mol_type = unassigned DNA
atgaaacgcctaattgactttacaaacatctatcagcgatcaaagactttgaggtttcgattggagcctatcggtaaaacggccgactatattaagaattctcagtccctcgaaactgatgcgcgtttggcaaaagagagcaagaaggtaaaagagcttgctgatgaatatcacaaagagtttattggagatgtcctgtcttcgttggaattgcctttaagcaaaatcaacgagttatgggatatatatatatatatatatatgtccaatgatacagaccgcgagataaaattcaaaaaactgcaagagaacctgcgaaaggtgattgcagaggcttttagtaaggacaaacggtttggtaatttattcaaaaaggagataatcacagacattctgccggaattcttgcaagataaggatgatgatattaagatcgtaaatagattcaaaggatttaccacatatttttacgcctttcataagaatagggaaaatatgtatgtctcggaagagaaatcgactgcaataccatatcgaattgtgaatcaaaatctcgtcaagtattttgacaactacaagacgttcaaagagaaggtaatgcctcttctgaaagacaagaatatagtcgaaagcatagagagagacttcaaagacatcttgaacgaaaaatcaatagaggatgtttttggccttgccaacttcactcatactttatgtcaggctgacatcgagaaatacaatacgttgataggtggccttgtcgtcaaaaacgaaaaaaaagagattaaaggtattaatcagtacattaacgaacataaccaaacgagtaaaaaagggaatggaattccgaaactaaagccgttgttcaatcagattttgagcgatagaaaatcgttatcgtttaccttagacgatatcaaaaaaacgtcggaggctattcgcaccattaaggatgagtatgaaaatctccgagacaagttggcgaccatcgaaaggcttattaagtctatcaaggagtatgatcttgcaggtatttacatcaagatgggagaggatacttcgacaatatcgcagcattggtttggtgcgtattataaaatcatcgaagcgatagcagatgcatgggaacgacgaaatccgaagaaaaacagagaatccaaggcatatagcaagtatgtatcgtccctaaaaagcatcagtctccaagaaatagatgatctcaaaatcggagagcctatagagaactacttcgcaacttttggcacgacttgttcagaccgaacaagtggagtttcttcgctcaataggataaaagctgcttataccgagttcgtgaacaaatttcctgaaggatttgaagatggcgatgactgtaacgatgcctactttaaggctaatgtggaagtcgtcaaaaatctgctggattcaattaaagattttcagcgttttgtgaagcctttgcttggcaatgaggacgaaagagacaaagacgaggcattctatggagagtttgtcccgacatacacagatatggataacatcataacccctctatacaaccgtgtacgcaattttgccaccaagaaaccatactctacagacaagataaaaatcaactttgaaaacgtagtattgctaaaaggatgggacaaaaacaaggagtcagactacgcatccatcatattgatgaaagacggacaatactttttaggggtactccgtaatggttcaaaaagtactcttaaaaccatattgcctaacacaggtgattgctatcaaaaaatggtttataagtattttaaggatataaaatcaaatcttccccggtgtacgacccagaggaaagacgtgaaagcgcactttgccgaatcgagcgacgattacactcttttagatacaaaggcctttgtttcggcactgactatcagcagagaagtgttcgaactatacaatgcccccgataaggagaaaaaattcaaaaaggaatatttgaagaacacaaacgatagtataggctacgccaatgctgtatccgtatgtaaacgcttctgtttggagttcctaaaaaaatatcgcagcactgccatatatgatctttcggatgttgaaacttcagtcgattcgtttgacgatttgtcctcattctatcaagagatagacaaaaggctgtacagcatctcattcgaaaatgtatctgtcgattccgtcaatgagcttgtagacaatggcaatatgcttctattccgtatcgcgaataaagatttttcgcctaacagcaagggccgtcccaatcttcatactatatattggcgaatgcttttcgacccggccaacctgaaggatgttgtatatcagctcaatggtaatgccgaaatattcttccgtaaggcaagcgttacgaggacggagcctacacatccggctaacgttgccatcaaaaacaagagcgaatataacaaacagaataagccgtatagtacattcaagtacggtttaatcaaggataggcgctacactaccgaccagttcgagtttcatgtacccatcacaatgaacttcaagcaaccagagtcgtctaaactacaggacaagctcaacaagcaagtgcttgacttcttgaaacaggacggcgtacgccatattataggcattgatcggggcgaacgtaatctgctatacttggtgatggtagatatggagggcaaaatcaaaaaacaaatatcactcaacgagatagccggtaatccgaagaatcccgagttcaaacaagacttccttgcactactgcacgagcgcgaaggtgaccgtttggagtcacgtcgcagttggaacaccattcagagcattaaagaactcaaagaaggttacatgagcttggtggttcatgaaatagcgaatatgatgcttgagaatgatgctatagtagtgctcgaaaatctgaatcgctcgtttatgcaaaagcgcggcggcatagaaaagtctgtataccaaaagttcgaaaagatgcttatcgacaagttgggatacatcgtggataagactaaagatgtgtccgacaacggaggcgcactacatgctgtacagcttgctgatacgtttgaaaacttcaataagacccaaaaaggagctattcgtcaatgtggattcatattctatattcctgcatggcgtaccagcaagattgaccccgttaccggctttgtgccaatgcttaggtgtcaatatgaaagcatcgtagaatccaaaaaattcttcggaaagttcgacagtatatactacgatgcgacaggaaagtattttgtcttccaaactgactttaccaaattcaataccgagagcaaaggaggaatccaaaaatgggatatatgcacctatggagacagaatatatgctcctcgcaccaaagaccggaataataaccctgtttcggaacgtgtaaaccttactgaggagatgaaatcactgtttgtatcgcataatatcaatattcaaggcgatatcaaagccggaattatgcagcagacagacaaggagttcttcgagtcactgcatcgattgcttcgacttacgttgcaaatacgcaatagcaaaaaatctacaggcaaagactatgaagactatatcatatcgccggtgatgggcaaggacggtcgtttctttgattcgcgtaacgcggatgctacgcaacctaaggatgcagatgccaatggcgcgtacaatattgcacgcaaaggcttgatgctgcttcgccagattcaagcccaagagaagcaagacctatccaacggaaaatggcttgaatttgcccaaaggtga

SEQ ID NO: 41 - DNA, 3810 bp, synthetic construct
  source 1..3810: mol_type = other DNA
aagcggctcatcgacttcaccaacatctaccagcgttctaagaccctgagattcagactggaacctatcggcaagaccgcggactacatcaaaaacagccagtccctggaaacagacgccagactggccaaggaatccaagaaagtgaaggaactggccgatgagtaccacaaagagtttatcggcgacgtgctgagcagcctggagctgcccctgagcaaaatcaacgagctgtgggacatctatatctacatctacatgagcaacgacaccgatcgggaaatcaaatttaagaagctccaggagaacctgcggaaggtgatcgccgaggcctttagcaaggataagagattcggcaacctgttcaagaaagaaatcatcacagatatcctgcccgagttcctgcaagataaagatgacgatatcaaaatcgtgaaccggttcaagggttttacaacctacttctacgccttccacaagaatcgggaaaacatgtacgtgtctgaagagaagagcacagccatcccctacagaatcgtgaatcaaaacctggtgaaatacttcgataactacaagacttttaaggagaaggtgatgcctctgctgaaggacaagaacatcgtcgaaagcatcgagcgcgacttcaaggacatcctgaacgagaaaagcatcgaggacgtgttcggcctggccaatttcacccacaccctgtgccaggctgacatcgagaagtacaacaccttgataggcggactggtggtgaagaacgaaaagaaggagatcaagggcatcaaccagtatattaacgagcacaaccagacctctaagaagggcaacggcatcccaaagctgaagcctctgtttaaccagatcctgagcgacagaaaatctctcagcttcaccctggatgatatcaagaaaaccagcgaggccatcagaacaattaaggacgagtatgagaacctgagagataagctggccacaatcgaacggctgatcaagagcatcaaggaatacgacctggccggcatctacatcaagatgggcgaggacacctctaccatctcccagcactggttcggtgcctattacaagattatcgaagccatcgccgacgcctgggagagaagaaacccaaagaaaaacagagagagcaaggcctacagcaagtacgtgagcagccttaagagcatcagcctgcaggagatcgacgacctgaagatcggcgagcctatcgagaattacttcgccacctttggaacaacatgtagcgaccggacatctggcgtgagctctctgaaccggatcaaagccgcctacaccgagttcgtgaacaagttccccgagggctttgaggatggcgatgattgcaacgacgcttacttcaaagccaatgtggaggtggtgaagaacttgctggatagcataaaagacttccagagatttgtgaagcctctactgggcaatgaggacgagcgggacaaagatgaggccttctacggcgagttcgttcctacctacacagatatggacaacatcatcacgcctctgtataatagagtcagaaacttcgctaccaagaagccttacagtacagacaagatcaaaataaacttcgaaaacgtggtactgctgaagggctgggataagaacaaggagagcgactatgccagcatcatcctgatgaaggacggccagtactttctgggagtgctgagaaacggatctaagagcactctgaaaaccatcctgcctaacaccggtgactgctaccagaaaatggtgtacaagtatttcaaggatatcaagtctaacctgcccagatgcaccacccagagaaaggacgtgaaggcacatttcgctgaaagcagcgatgattacaccctgcttgatacaaaagccttcgtgagcgctctgacgatctccagagaggtgttcgaactgtacaacgctcctgataaggaaaagaaattcaagaaggaatacctgaagaacaccaacgactccatcggctacgccaatgcagtgagcgtgtgcaagagattctgcctggagttcctgaaaaagtaccggagcaccgccatctacgacctgagcgatgttgaaacctctgtggacagtttcgacgacctgagcagcttctaccaggagatcgataagagactgtacagcatcagcttcgaaaacgtgagcgtggacagcgtgaacgagctggtggataacggcaatatgctgctgttcagaatcgccaacaaggatttctctcctaatagcaagggcagacctaatctgcacacaatttactggagaatgctgttcgaccctgctaatctcaaggacgtcgtgtaccaactgaacggcaatgccgaaatcttcttccggaaggccagcgttacaaggacagaaccaacacaccccgccaatgtggccatcaagaacaagagcgagtacaacaagcagaacaaaccttacagcaccttcaagtacggcctcatcaaggaccggcgatacaccaccgatcagttcgagttccacgtgcctatcaccatgaacttcaagcaacctgagtcatctaagctgcaggacaaactgaataagcaagtgctggacttcctgaagcaagacggcgtgcggcacatcatcggcatcgaccggggagaaagaaacctgctgtacctggtgatggtcgacatggaaggaaaaatcaagaagcagatcagcctgaatgaaatcgccggaaacccaaagaaccctgagtttaagcaggacttcttagctctgctgcatgagagagagggcgatagactggagtccagaagaagttggaacaccatccagagcatcaaggagctgaaagaaggctacatgtccctggtggtgcacgagatcgctaacatgatgctggagaatgatgccatcgtggtcttggaaaaccttaacagatcctttatgcagaagagaggcggcattgagaaaagcgtgtaccagaagtttgagaaaatgctgatcgacaagctgggctacatcgtggacaaaacaaaagatgtgtcagataatggcggagccctgcacgccgtgcagctggctgacaccttcgagaactttaacaagacccagaaaggcgccatccggcagtgcggcttcatcttttatatccccgcctggcggacaagcaaaattgacccggtaaccggctttgtgcccatgctgagatgtcagtacgaatctatcgtggaatccaagaagttctttggcaaattcgactctatctactacgacgccaccggaaagtacttcgtgttccagaccgactttaccaagttcaacaccgagtctaaggggggcatccagaagtgggacatctgtacctacggagacagaatctacgcccctagaaccaaagacagaaataacaaccctgtgtccgaaagagtgaacctgacagaagaaatgaagagcctgttcgtaagccacaatatcaacatccagggcgacatcaaggccggcattatgcagcagacagacaaggagttcttcgagtcgctgcacagactgctgagactgaccctgcagatccggaacagcaagaaaagcaccggcaaggactacgaggactacattatcagtcctgtgatgggcaaggacggaagattcttcgacagccggaacgccgacgccacccagcccaaggacgccgacgcaaacggcgcctacaacattgccagaaaaggcctgatgctgctgcgccagatccaggcccaggagaagcaggacctgtctaatgggaagtggctggagttcgcccagcgg

SEQ ID NO: 42 - DNA, 3927 bp, synthetic construct
  source 1..3927: mol_type = other DNA
atgggcaagcggctcatcgacttcaccaacatctaccagcgttctaagaccctgagattcagactggaacctatcggcaagaccgcggactacatcaaaaacagccagtccctggaaacagacgccagactggccaaggaatccaagaaagtgaaggaactggccgatgagtaccacaaagagtttatcggcgacgtgctgagcagcctggagctgcccctgagcaaaatcaacgagctgtgggacatctatatctacatctacatgagcaacgacaccgatcgggaaatcaaatttaagaagctccaggagaacctgcggaaggtgatcgccgaggcctttagcaaggataagagattcggcaacctgttcaagaaagaaatcatcacagatatcctgcccgagttcctgcaagataaagatgacgatatcaaaatcgtgaaccggttcaagggttttacaacctacttctacgccttccacaagaatcgggaaaacatgtacgtgtctgaagagaagagcacagccatcccctacagaatcgtgaatcaaaacctggtgaaatacttcgataactacaagacttttaaggagaaggtgatgcctctgctgaaggacaagaacatcgtcgaaagcatcgagcgcgacttcaaggacatcctgaacgagaaaagcatcgaggacgtgttcggcctggccaatttcacccacaccctgtgccaggctgacatcgagaagtacaacaccttgataggcggactggtggtgaagaacgaaaagaaggagatcaagggcatcaaccagtatattaacgagcacaaccagacctctaagaagggcaacggcatcccaaagctgaagcctctgtttaaccagatcctgagcgacagaaaatctctcagcttcaccctggatgatatcaagaaaaccagcgaggccatcagaacaattaaggacgagtatgagaacctgagagataagctggccacaatcgaacggctgatcaagagcatcaaggaatacgacctggccggcatctacatcaagatgggcgaggacacctctaccatctcccagcactggttcggtgcctattacaagattatcgaagccatcgccgacgcctgggagagaagaaacccaaagaaaaacagagagagcaaggcctacagcaagtacgtgagcagccttaagagcatcagcctgcaggagatcgacgacctgaagatcggcgagcctatcgagaattacttcgccacctttggaacaacatgtagcgaccggacatctggcgtgagctctctgaaccggatcaaagccgcctacaccgagttcgtgaacaagttccccgagggctttgaggatggcgatgattgcaacgacgcttacttcaaagccaatgtggaggtggtgaagaacttgctggatagcataaaagacttccagagatttgtgaagcctctactgggcaatgaggacgagcgggacaaagatgaggccttctacggcgagttcgttcctacctacacagatatggacaacatcatcacgcctctgtataatagagtcagaaacttcgctaccaagaagccttacagtacagacaagatcaaaataaacttcgaaaacgtggtactgctgaagggctgggataagaacaaggagagcgactatgccagcatcatcctgatgaaggacggccagtactttctgggagtgctgagaaacggatctaagagcactctgaaaaccatcctgcctaacaccggtgactgctaccagaaaatggtgtacaagtatttcaaggatatcaagtctaacctgcccagatgcaccacccagagaaaggacgtgaaggcacatttcgctgaaagcagcgatgattacaccctgcttgatacaaaagccttcgtgagcgctctgacgatctccagagaggtgttcgaactgtacaacgctcctgataaggaaaagaaattcaagaaggaatacctgaagaacaccaacgactccatcggctacgccaatgcagtgagcgtgtgcaagagattctgcctggagttcctgaaaaagtaccggagcaccgccatctacgacctgagcgatgttgaaacctctgtggacagtttcgacgacctgagcagcttctaccaggagatcgataagagactgtacagcatcagcttcgaaaacgtgagcgtggacagcgtgaacgagctggtggataacggcaatatgctgctgttcagaatcgccaacaaggatttctctcctaatagcaagggcagacctaatctgcacacaatttactggagaatgctgttcgaccctgctaatctcaaggacgtcgtgtaccaactgaacggcaatgccgaaatcttcttccggaaggccagcgttacaaggacagaaccaacacaccccgccaatgtggccatcaagaacaagagcgagtacaacaagcagaacaaaccttacagcaccttcaagtacggcctcatcaaggaccggcgatacaccaccgatcagttcgagttccacgtgcctatcaccatgaacttcaagcaacctgagtcatctaagctgcaggacaaactgaataagcaagtgctggacttcctgaagcaagacggcgtgcggcacatcatcggcatcgaccggggagaaagaaacctgctgtacctggtgatggtcgacatggaaggaaaaatcaagaagcagatcagcctgaatgaaatcgccggaaacccaaagaaccctgagtttaagcaggacttcttagctctgctgcatgagagagagggcgatagactggagtccagaagaagttggaacaccatccagagcatcaaggagctgaaagaaggctacatgtccctggtggtgcacgagatcgctaacatgatgctggagaatgatgccatcgtggtcttggaaaaccttaacagatcctttatgcagaagagaggcggcattgagaaaagcgtgtaccagaagtttgagaaaatgctgatcgacaagctgggctacatcgtggacaaaacaaaagatgtgtcagataatggcggagccctgcacgccgtgcagctggctgacaccttcgagaactttaacaagacccagaaaggcgccatccggcagtgcggcttcatcttttatatccccgcctggcggacaagcaaaattgacccggtaaccggctttgtgcccatgctgagatgtcagtacgaatctatcgtggaatccaagaagttctttggcaaattcgactctatctactacgacgccaccggaaagtacttcgtgttccagaccgactttaccaagttcaacaccgagtctaaggggggcatccagaagtgggacatctgtacctacggagacagaatctacgcccctagaaccaaagacagaaataacaaccctgtgtccgaaagagtgaacctgacagaagaaatgaagagcctgttcgtaagccacaatatcaacatccagggcgacatcaaggccggcattatgcagcagacagacaaggagttcttcgagtcgctgcacagactgctgagactgaccctgcagatccggaacagcaagaaaagcaccggcaaggactacgaggactacattatcagtcctgtgatgggcaaggacggaagattcttcgacagccggaacgccgacgccacccagcccaaggacgccgacgcaaacggcgcctacaacattgccagaaaaggcctgatgctgctgcgccagatccaggcccaggagaagcaggacctgtctaatgggaagtggctggagttcgcccagcggtctagaaagcggacagcagacggctccgaatttgaaagccctaagaaaaagagaaaggtgggatccggcaaacctatccccaatcccctgctgggcctggacagcacctga

SEQ ID NO: 43 - protein, 1219 aa, unidentified
AEMFKDFTNLYPVSKTLRFELIPEGETLHYLEKNGVLENDEKRNEDYKKLKKLMDEYYRAYIDEALSNVHLSDLDRYAELYSIQNKSDEENVEFENVQLRLRTQIVGFLESRETYSSLFKKELIEKELPKFFIRREEELNLIKSFKGFTTMCTGFWENRKNMFSAEEKSTAIAYRVVHENLPKFMNNIRIFRLFIDEKLDCSEKLLEKAGVNSLSEVFELDYFNNTLSQRGIELYNCILGGFTEDEKHKIQGVNELINLYNQQTKEKKIPQLQPLYKQILSDTKSLSFLADAFENDGGVLATVKALYDEFHEEILSERGLISTTLQNIEKYDSKGIFVKNDLTITGLSNSLFGDWKAINGSLNSWYEENVPRKERTEEKHVEVRKAYFKKLKSISLEFIEEAGLSELRCKYKALLLEKAEAVCDAYKNAEELFSEAYNENTNLIADGKSVEKIKALLDSMKELEAVILMLSGTGEEAERDELFYGEFEKHRFVLNLLDNVFNKTRNYVTKKPYKTEKIKLTFDSPTLLDGWDRNKETSNKSVILMKDGYYYLGIMNKANNKAFENLKDTGGKCYSKMDYKLLPGPNKMLPKVFFAKKNIDYYAPSEDLLQKYKEGTHKKGKKFNLEDCHALIDFFKDSIAKHPEWNEFGFDFSDTKSYRDISDFYKEVSEQGYKISYRNVSVNYIDSLVREGKLYLFKIYNKDFSPYSKGRPNLHTMYWKALFANKNFENRIYKLNGQAEMFYRKKSIPEDKRVIHSAKEPIDQRRNTDEKSLFDYDIIKDRRYTVDKFQFNVPITMNYTAPGSGRINRKMREAIKNCENMHIIGIDRGERHLLYVTVIDMQGNIKEQFSLNRILSEYKANNVAKSVETDYKTLLTKKEIERQDARKQWKSIENIKELKDGYMSQVVHVIAELMIKYNAIVVMEDLNFGFKRGRQKVERQVYQKFEKALIDKLNYLVDKTASEMENTGLYAALQLTEKFESFKKMGKQNGGLFYVNAWNTSKMDPTTGFVNLLYPKYESIEKSKAYIEKFKDIQFCDDDEYGKYLAISFDYNDFTEKAKGAKTEWTICSYGKRLYNHRNKDGYWEEQELDLTEEYFNLFEEFGINAASNIKEQVIAQNSADFFRRFMWLLKMTLQIRNSETNGETDYMLSPVKNEDGKFFNSDEVKDDTLPENADANGAYNIARKGLLLVERIKDCPDEELDKVDLKVTNLDWMKFAQR

SEQ ID NO: 44 - protein, 1220 aa, unidentified
MAEMFKDFTNLYPVSKTLRFELIPEGETLHYLEKNGVLENDEKRNEDYKKLKKLMDEYYRAYIDEALSNVHLSDLDRYAELYSIQNKSDEENVEFENVQLRLRTQIVGFLESRETYSSLFKKELIEKELPKFFIRREEELNLIKSFKGFTTMCTGFWENRKNMFSAEEKSTAIAYRVVHENLPKFMNNIRIFRLFIDEKLDCSEKLLEKAGVNSLSEVFELDYFNNTLSQRGIELYNCILGGFTEDEKHKIQGVNELINLYNQQTKEKKIPQLQPLYKQILSDTKSLSFLADAFENDGGVLATVKALYDEFHEEILSERGLISTTLQNIEKYDSKGIFVKNDLTITGLSNSLFGDWKAINGSLNSWYEENVPRKERTEEKHVEVRKAYFKKLKSISLEFIEEAGLSELRCKYKALLLEKAEAVCDAYKNAEELFSEAYNENTNLIADGKSVEKIKALLDSMKELEAVILMLSGTGEEAERDELFYGEFEKHRFVLNLLDNVFNKTRNYVTKKPYKTEKIKLTFDSPTLLDGWDRNKETSNKSVILMKDGYYYLGIMNKANNKAFENLKDTGGKCYSKMDYKLLPGPNKMLPKVFFAKKNIDYYAPSEDLLQKYKEGTHKKGKKFNLEDCHALIDFFKDSIAKHPEWNEFGFDFSDTKSYRDISDFYKEVSEQGYKISYRNVSVNYIDSLVREGKLYLFKIYNKDFSPYSKGRPNLHTMYWKALFANKNFENRIYKLNGQAEMFYRKKSIPEDKRVIHSAKEPIDQRRNTDEKSLFDYDIIKDRRYTVDKFQFNVPITMNYTAPGSGRINRKMREAIKNCENMHIIGIDRGERHLLYVTVIDMQGNIKEQFSLNRILSEYKANNVAKSVETDYKTLLTKKEIERQDARKQWKSIENIKELKDGYMSQVVHVIAELMIKYNAIVVMEDLNFGFKRGRQKVERQVYQKFEKALIDKLNYLVDKTASEMENTGLYAALQLTEKFESFKKMGKQNGGLFYVNAWNTSKMDPTTGFVNLLYPKYESIEKSKAYIEKFKDIQFCDDDEYGKYLAISFDYNDFTEKAKGAKTEWTICSYGKRLYNHRNKDGYWEEQELDLTEEYFNLFEEFGINAASNIKEQVIAQNSADFFRRFMWLLKMTLQIRNSETNGETDYMLSPVKNEDGKFFNSDEVKDDTLPENADANGAYNIARKGLLLVERIKDCPDEELDKVDLKVTNLDWMKFAQR

SEQ ID NO: 45 - protein, 1257 aa, synthetic construct
MGAEMFKDFTNLYPVSKTLRFELIPEGETLHYLEKNGVLENDEKRNEDYKKLKKLMDEYYRAYIDEALSNVHLSDLDRYAELYSIQNKSDEENVEFENVQLRLRTQIVGFLESRETYSSLFKKELIEKELPKFFIRREEELNLIKSFKGFTTMCTGFWENRKNMFSAEEKSTAIAYRVVHENLPKFMNNIRIFRLFIDEKLDCSEKLLEKAGVNSLSEVFELDYFNNTLSQRGIELYNCILGGFTEDEKHKIQGVNELINLYNQQTKEKKIPQLQPLYKQILSDTKSLSFLADAFENDGGVLATVKALYDEFHEEILSERGLISTTLQNIEKYDSKGIFVKNDLTITGLSNSLFGDWKAINGSLNSWYEENVPRKERTEEKHVEVRKAYFKKLKSISLEFIEEAGLSELRCKYKALLLEKAEAVCDAYKNAEELFSEAYNENTNLIADGKSVEKIKALLDSMKELEAVILMLSGTGEEAERDELFYGEFEKHRFVLNLLDNVFNKTRNYVTKKPYKTEKIKLTFDSPTLLDGWDRNKETSNKSVILMKDGYYYLGIMNKANNKAFENLKDTGGKCYSKMDYKLLPGPNKMLPKVFFAKKNIDYYAPSEDLLQKYKEGTHKKGKKFNLEDCHALIDFFKDSIAKHPEWNEFGFDFSDTKSYRDISDFYKEVSEQGYKISYRNVSVNYIDSLVREGKLYLFKIYNKDFSPYSKGRPNLHTMYWKALFANKNFENRIYKLNGQAEMFYRKKSIPEDKRVIHSAKEPIDQRRNTDEKSLFDYDIIKDRRYTVDKFQFNVPITMNYTAPGSGRINRKMREAIKNCENMHIIGIDRGERHLLYVTVIDMQGNIKEQFSLNRILSEYKANNVAKSVETDYKTLLTKKEIERQDARKQWKSIENIKELKDGYMSQVVHVIAELMIKYNAIVVMEDLNFGFKRGRQKVERQVYQKFEKALIDKLNYLVDKTASEMENTGLYAALQLTEKFESFKKMGKQNGGLFYVNAWNTSKMDPTTGFVNLLYPKYESIEKSKAYIEKFKDIQFCDDDEYGKYLAISFDYNDFTEKAKGAKTEWTICSYGKRLYNHRNKDGYWEEQELDLTEEYFNLFEEFGINAASNIKEQVIAQNSADFFRRFMWLLKMTLQIRNSETNGETDYMLSPVKNEDGKFFNSDEVKDDTLPENADANGAYNIARKGLLLVERIKDCPDEELDKVDLKVTNLDWMKFAQRSRKRTADGSEFESPKKKRKVGSGKPIPNPLLGLDST

SEQ ID NO: 46 - DNA, 3663 bp, unidentified
  source 1..3663: mol_type = unassigned DNA
atggctgagatgtttaaagattttacgaatttgtatcctgtttcaaaaaccttgcgttttgaattaattcctgaaggggaaacattgcattatcttgaaaaaaatggcgttctggaaaacgatgagaagcgaaacgaagattataagaagttgaaaaaactgatggatgaatattaccgtgcatacatcgatgaagctttatctaatgttcatctttcagatttggatagatatgcagaattatattcaattcagaataaatcggatgaagaaaatgtagaattcgaaaatgttcaactgagattgagaacacaaattgttggattcttagaatccagagaaacctattcttcacttttcaaaaaagaactgattgagaaggaacttcctaaattctttattcggagagaagaggagcttaatttaatcaaatcatttaaaggttttacaacgatgtgcaccggcttctgggaaaatcggaaaaatatgttttctgccgaagaaaaatctacagcaatagcatatcgtgtagtccatgaaaacctacctaagtttatgaataatataagaatttttcgtttgttcattgatgaaaagttggactgttctgaaaaattgctggaaaaagccggagtgaattctctgagtgaagtgtttgaacttgattattttaacaatacattatcccaacgtggcattgaattgtataactgtatattgggcggatttaccgaggatgaaaagcataagattcaaggcgtaaacgaattgattaatttgtacaatcagcagacaaaagagaagaagattccacagttgcagccgctgtacaagcagattctcagcgataccaagagcctttcatttcttgcagatgcatttgaaaacgacgggggggtcttagcgactgtaaaagcattatatgatgaatttcatgaagagattttgagcgaaaggggattaatctctacgacattacagaatattgaaaagtatgattcaaaaggcatcttcgtaaaaaacgatttaacgattaccggtttatcaaatagtttgttcggcgactggaaggctattaatggtagtttaaattcgtggtatgaggagaacgtgcctcgaaaagaaagaactgaagagaaacatgtagaggtaagaaaagcctattttaaaaagttaaaatcaataagcctggaatttatcgaggaggccggattgtcggaactccgttgcaaatataaagcccttcttttagaaaaagcagaggctgtttgcgatgcgtacaaaaatgcagaagagctttttagtgaagcttataatgaaaatactaaccttattgccgatggaaagtctgtggaaaaaataaaagcgctattggattctatgaaagagcttgaagcggtgattcttatgctttccggaaccggagaggaagcagaacgggatgaattgttttacggcgaatttgaaaaacataggttcgtattgaatctcttagacaacgtatttaataaaacgagaaattacgtaacaaagaaaccatataagactgagaagattaaattaacatttgattccccaacgctgctagacgggtgggatcgtaataaagaaacatcaaacaagtccgtgatacttatgaaagatggctattattaccttggaattatgaacaaggcaaataacaaagcctttgagaatttgaaagacacaggcgggaaatgctatagcaagatggattacaaacttttgcctggaccaaacaagatgttgccgaaggtgttttttgcaaagaaaaacatcgactattatgcaccaagcgaagacttgctacagaaatataaagagggaacacataaaaaaggaaagaaatttaatctagaggattgtcacgcgttaatagacttttttaaagactcaattgcaaagcatccagaatggaacgagtttggatttgatttttcagatacgaaatcatatcgagatattagtgatttctataaggaggtttcagagcagggatacaaaatcagttatcgaaatgtatctgttaattacatagattctctagtaagagaagggaaattgtatttgttcaaaatttataataaagatttttcaccgtacagcaaaggcagaccaaatcttcatacgatgtattggaaagcgttattcgctaataagaattttgaaaatcgcatatataagttaaatggccaggcagaaatgttctatcgaaaaaagagcattccggaagacaagagggtgattcactcggcaaaagaaccaatcgatcagagaagaaatacggatgaaaagagcctctttgattatgacattattaaagatcggcgatatactgtggacaaattccaatttaatgttccgattacgatgaattacactgcaccgggttccggccgaattaacagaaaaatgcgggaagcgattaagaactgtgaaaatatgcatattatcggaatagatagaggcgaacgtcatttgctgtatgtgacggttatcgatatgcagggaaacattaaagaacagttttcattaaatcgaatcctgagtgagtacaaggcaaacaatgtggctaaaagtgtcgaaacggactacaaaacactcctgacaaaaaaagaaattgaacgacaggatgcaagaaagcagtggaagagcattgaaaatattaaggaattaaaagacggctacatgagccaggttgtgcatgtgattgccgaactcatgataaagtacaatgcgattgtggttatggaggatttgaatttcggattcaagcgaggaagacagaaggttgagagacaggtttaccagaagtttgagaaggcattaattgataaattgaactatttggttgataaaacagcctctgaaatggagaacaccggtctgtatgcggcattgcagcttacagaaaaatttgagagctttaagaaaatgggcaaacaaaatggtggattattttatgtaaacgcatggaataccagtaaaatggatccaacaaccggttttgtgaaccttctctatcctaaatatgagagcattgaaaaaagcaaagcgtatattgagaaattcaaggatattcagttttgtgatgatgacgaatatggaaagtaccttgcaatatcttttgattataacgatttcacggagaaggcaaagggcgcaaaaacggaatggaccatttgctcttatggaaagagattgtataatcacagaaataaagatgggtattgggaagagcaggaattggatcttacagaagagtatttcaatctgtttgaagaatttggaattaatgcagcgtctaatattaaagaacaagtcatcgcacagaattctgcagacttttttagacggtttatgtggcttttgaaaatgaccttacagattagaaacagtgaaacaaatggggagacggattatatgctttctccggtaaaaaatgaagacggaaaattctttaattcagatgaagtcaaggatgacacgcttccggaaaatgcggatgcgaatggtgcatacaacatcgctagaaaaggattactgcttgtggaaagaattaaagactgtccggacgaagaacttgataaggttgatttgaaggtaacaaatttagattggatgaaatttgcacagaggtaa

SEQ ID NO: 47 - DNA, 3657 bp, synthetic construct
  source 1..3657: mol_type = other DNA
gccgaaatgttcaaggacttcaccaacctgtacccagtgtccaaaaccctccggttcgaattgatccccgagggcgaaacactgcactacctagaaaagaacggagtgctggaaaacgacgagaagagaaatgaggattacaagaagctgaagaaactcatggatgaatactaccgggcctacatcgacgaggccttatctaatgtccacctgtccgatctggaccggtacgccgaactgtattctatccagaacaagagcgatgaggagaacgtggagttcgagaatgtgcagctgcgcctgagaacccagatcgtgggcttcctggaaagcagagaaacctacagcagcctgttcaagaaggagctgatcgaaaaagaactgcctaagtttttcatcagaagagaggaagagctgaacctgataaagagctttaagggctttaccactatgtgcaccggcttctgggaaaatcggaagaacatgttcagcgccgaggaaaagtccacagccatcgcctatagagtggtccatgaaaacctgcccaagttcatgaacaacattagaatcttccggctgtttatcgacgagaagctggattgtagcgagaagctgctggagaaggccggcgtgaacagcctgagcgaggtgttcgagcttgactatttcaataacaccctgagccagagaggcatcgagctgtacaactgcatcctgggcggattcaccgaggatgaaaaacacaagatccagggagtgaacgagttgatcaacctgtacaaccagcagacaaaggagaagaaaattcctcagctgcaacctctgtacaaacagatcctgtctgacacgaagtcgctgtcctttctggctgatgcctttgaaaacgacggaggagtgctggctacagtgaaggctttatatgatgagtttcacgaggaaatcctgagcgagagaggcctgatcagcacaaccctgcagaacattgagaagtacgatagtaagggcatctttgttaagaacgatctcaccattacaggcctgtccaacagcctgtttggagattggaaggccatcaatggaagcctgaacagctggtacgaggagaacgtgccccggaaggagcgaacagaagagaaacacgtggaagtgagaaaggcttattttaagaagctgaagtctatcagcctggagttcatcgaggaggccggactgagcgagctgcggtgcaagtacaaggccctgctgctggagaaagccgaggctgtgtgcgacgcgtacaagaacgccgaggagctgtttagcgaggcctataatgagaacaccaatctgatcgccgatggcaaatctgtggaaaaaatcaaagccctgctggacagcatgaaggagctggaggccgtgatcctgatgctgagcggcacaggcgaggaggccgagcgggacgaactgttttatggcgagttcgaaaaacatagattcgtgctgaatctgctggacaacgtgttcaacaagaccagaaactacgtgaccaagaagccttacaagaccgagaagatcaagctcaccttcgacagccctacccttctggatggctgggaccgtaacaaggagacaagcaacaagagcgtgatcctgatgaaggatggctactactacctgggcatcatgaacaaagccaacaacaaggccttcgagaacctgaaggacacaggaggcaaatgctacagcaagatggactacaagctgctgcctggccctaacaagatgctgcctaaggtgttctttgccaaaaagaacatcgactactacgcccctagcgaggacctgctgcagaagtacaaggagggcacccacaagaaagggaagaagttcaatcttgaggactgtcacgccctgatcgacttcttcaaggacagcatcgctaaacaccccgagtggaacgagttcggcttcgacttttctgacaccaagtcttatagagacatctcggatttctacaaggaggtcagcgaacagggctacaagattagctaccggaacgtgagtgttaactacatcgacagtctggtgcgggaaggtaagctgtacctgttcaagatctacaacaaggacttcagcccatactccaaaggacgtcccaacctgcacaccatgtactggaaagccctgttcgccaataaaaacttcgaaaaccggatctacaagctgaacggccaggccgaaatgttctacagaaagaaatctatccctgaagataagcgggtgatccacagcgccaaagaacctatcgatcagagaagaaacaccgacgaaaagtctctgtttgactacgacatcatcaaggacagacggtacaccgtggacaagttccagttcaacgtgccaatcacaatgaactacaccgcccctggcagcggcagaatcaacagaaagatgcgggaagctatcaagaattgcgagaatatgcacatcatcggcatcgaccggggagagcggcacctgctgtacgtgaccgtgatcgacatgcagggcaacatcaaagaacagttctctctcaaccgcatcctgtctgagtacaaggccaataacgtcgccaagagcgtggagacagactacaaaacactgctgacgaaaaaagagatcgagagacaggacgctagaaagcaatggaagagcatcgaaaacatcaaagagctgaaagacggctatatgagccaggtggtgcacgtgatagcagagctgatgatcaagtacaacgccatagttgtgatggaggacctgaatttcggcttcaagagaggccggcaaaaggtggagagacaggtgtaccagaaattcgagaaggccctgatcgataagctgaattacctggtggataagacagcttccgagatggaaaacaccggcctgtacgccgccctgcagctgacagagaagttcgaatccttcaagaagatgggcaaacagaacggcggcttgttctacgtgaacgcctggaacaccagcaagatggaccctaccaccggattcgtgaacctgctgtaccctaagtacgaatctatcgaaaagagcaaggcctatatcgagaaattcaaggatatccagttttgtgacgacgatgaatacggcaaatacctggcaatttctttcgactacaacgacttcacagaaaaggccaagggcgccaagaccgagtggaccatctgcagctacggcaaaagactgtacaaccacagaaataaggacggctactgggaggagcaggagctggatctgaccgaggagtacttcaacctgttcgaagagttcggcatcaacgctgccagcaacatcaaggaacaagtgatcgctcagaacagcgccgatttcttcagaagattcatgtggctgctgaagatgaccctgcagatcaggaactctgaaactaacggcgaaaccgattacatgctgagccctgtgaagaacgaggacggcaaattcttcaactctgacgaggtgaaggacgacaccctgcccgagaatgccgacgccaacggcgcctacaacatcgcaagaaagggcctgctgctggtcgaacgtatcaaggattgccccgacgaagaactagacaaggtggacctgaaggtcaccaacctggactggatgaaattcgcccaaaga

SEQ ID NO: 48 - DNA, 3774 bp, synthetic construct
  source 1..3774: mol_type = other DNA
atgggcgccgaaatgttcaaggacttcaccaacctgtacccagtgtccaaaaccctccggttcgaattgatccccgagggcgaaacactgcactacctagaaaagaacggagtgctggaaaacgacgagaagagaaatgaggattacaagaagctgaagaaactcatggatgaatactaccgggcctacatcgacgaggccttatctaatgtccacctgtccgatctggaccggtacgccgaactgtattctatccagaacaagagcgatgaggagaacgtggagttcgagaatgtgcagctgcgcctgagaacccagatcgtgggcttcctggaaagcagagaaacctacagcagcctgttcaagaaggagctgatcgaaaaagaactgcctaagtttttcatcagaagagaggaagagctgaacctgataaagagctttaagggctttaccactatgtgcaccggcttctgggaaaatcggaagaacatgttcagcgccgaggaaaagtccacagccatcgcctatagagtggtccatgaaaacctgcccaagttcatgaacaacattagaatcttccggctgtttatcgacgagaagctggattgtagcgagaagctgctggagaaggccggcgtgaacagcctgagcgaggtgttcgagcttgactatttcaataacaccctgagccagagaggcatcgagctgtacaactgcatcctgggcggattcaccgaggatgaaaaacacaagatccagggagtgaacgagttgatcaacctgtacaaccagcagacaaaggagaagaaaattcctcagctgcaacctctgtacaaacagatcctgtctgacacgaagtcgctgtcctttctggctgatgcctttgaaaacgacggaggagtgctggctacagtgaaggctttatatgatgagtttcacgaggaaatcctgagcgagagaggcctgatcagcacaaccctgcagaacattgagaagtacgatagtaagggcatctttgttaagaacgatctcaccattacaggcctgtccaacagcctgtttggagattggaaggccatcaatggaagcctgaacagctggtacgaggagaacgtgccccggaaggagcgaacagaagagaaacacgtggaagtgagaaaggcttattttaagaagctgaagtctatcagcctggagttcatcgaggaggccggactgagcgagctgcggtgcaagtacaaggccctgctgctggagaaagccgaggctgtgtgcgacgcgtacaagaacgccgaggagctgtttagcgaggcctataatgagaacaccaatctgatcgccgatggcaaatctgtggaaaaaatcaaagccctgctggacagcatgaaggagctggaggccgtgatcctgatgctgagcggcacaggcgaggaggccgagcgggacgaactgttttatggcgagttcgaaaaacatagattcgtgctgaatctgctggacaacgtgttcaacaagaccagaaactacgtgaccaagaagccttacaagaccgagaagatcaagctcaccttcgacagccctacccttctggatggctgggaccgtaacaaggagacaagcaacaagagcgtgatcctgatgaaggatggctactactacctgggcatcatgaacaaagccaacaacaaggccttcgagaacctgaaggacacaggaggcaaatgctacagcaagatggactacaagctgctgcctggccctaacaagatgctgcctaaggtgttctttgccaaaaagaacatcgactactacgcccctagcgaggacctgctgcagaagtacaaggagggcacccacaagaaagggaagaagttcaatcttgaggactgtcacgccctgatcgacttcttcaaggacagcatcgctaaacaccccgagtggaacgagttcggcttcgacttttctgacaccaagtcttatagagacatctcggatttctacaaggaggtcagcgaacagggctacaagattagctaccggaacgtgagtgttaactacatcgacagtctggtgcgggaaggtaagctgtacctgttcaagatctacaacaaggacttcagcccatactccaaaggacgtcccaacctgcacaccatgtactggaaagccctgttcgccaataaaaacttcgaaaaccggatctacaagctgaacggccaggccgaaatgttctacagaaagaaatctatccctgaagataagcgggtgatccacagcgccaaagaacctatcgatcagagaagaaacaccgacgaaaagtctctgtttgactacgacatcatcaaggacagacggtacaccgtggacaagttccagttcaacgtgccaatcacaatgaactacaccgcccctggcagcggcagaatcaacagaaagatgcgggaagctatcaagaattgcgagaatatgcacatcatcggcatcgaccggggagagcggcacctgctgtacgtgaccgtgatcgacatgcagggcaacatcaaagaacagttctctctcaaccgcatcctgtctgagtacaaggccaataacgtcgccaagagcgtggagacagactacaaaacactgctgacgaaaaaagagatcgagagacaggacgctagaaagcaatggaagagcatcgaaaacatcaaagagctgaaagacggctatatgagccaggtggtgcacgtgatagcagagctgatgatcaagtacaacgccatagttgtgatggaggacctgaatttcggcttcaagagaggccggcaaaaggtggagagacaggtgtaccagaaattcgagaaggccctgatcgataagctgaattacctggtggataagacagcttccgagatggaaaacaccggcctgtacgccgccctgcagctgacagagaagttcgaatccttcaagaagatgggcaaacagaacggcggcttgttctacgtgaacgcctggaacaccagcaagatggaccctaccaccggattcgtgaacctgctgtaccctaagtacgaatctatcgaaaagagcaaggcctatatcgagaaattcaaggatatccagttttgtgacgacgatgaatacggcaaatacctggcaatttctttcgactacaacgacttcacagaaaaggccaagggcgccaagaccgagtggaccatctgcagctacggcaaaagactgtacaaccacagaaataaggacggctactgggaggagcaggagctggatctgaccgaggagtacttcaacctgttcgaagagttcggcatcaacgctgccagcaacatcaaggaacaagtgatcgctcagaacagcgccgatttcttcagaagattcatgtggctgctgaagatgaccctgcagatcaggaactctgaaactaacggcgaaaccgattacatgctgagccctgtgaagaacgaggacggcaaattcttcaactctgacgaggtgaaggacgacaccctgcccgagaatgccgacgccaacggcgcctacaacatcgcaagaaagggcctgctgctggtcgaacgtatcaaggattgccccgacgaagaactagacaaggtggacctgaaggtcaccaacctggactggatgaaattcgcccaaaggtctagaaagcggacagcagacggctccgaatttgaaagccctaagaaaaagagaaaggtgggatccggcaaacctatccccaatcccctgctgggcctggacagcacctga

SEQ ID NO: 49 - protein, 1200 aa, unidentified
KLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLEHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRRLNKRKSRTTDEEESRKLFKQKGSLSIGYIADTAKIDVERYFAKLGAINTVTEQSENLFAKAENARTTADELLANDYPAGKRLVQSNDDIALLKNLLDALMELQWFVKPLLGTGDEAGKDERFYGEFAQIWEQLDRITPLYNMVRNYVTRKPYSTDKFKLNFESAALLGGWDKNKEPDCLSVILRKDEQYYLGIINKNHKKIFENDILPCEGECYDKMVYKLLPGANKMLPKVFFSASRIAEFAPSDEVKRIYNDKTFQKGEKFDLNDCRTLIDFYKASIDKHEEWNKFGFEFSDTNNYEDISGFFREVDRQGYKMSFRPVAASYIETLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKTLLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMQRILKLTMQMRNSKTGTDTDYIVSPVADANGRFFDSRQADATMPKDADANGAYNIARKGIMLVQQIKQSDDLRTMKFDISNKSWLRFAQHTNQADE

SEQ ID NO: 50 - protein, 1201 aa, unidentified
MKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLEHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRRLNKRKSRTTDEEESRKLFKQKGSLSIGYIADTAKIDVERYFAKLGAINTVTEQSENLFAKAENARTTADELLANDYPAGKRLVQSNDDIALLKNLLDALMELQWFVKPLLGTGDEAGKDERFYGEFAQIWEQLDRITPLYNMVRNYVTRKPYSTDKFKLNFESAALLGGWDKNKEPDCLSVILRKDEQYYLGIINKNHKKIFENDILPCEGECYDKMVYKLLPGANKMLPKVFFSASRIAEFAPSDEVKRIYNDKTFQKGEKFDLNDCRTLIDFYKASIDKHEEWNKFGFEFSDTNNYEDISGFFREVDRQGYKMSFRPVAASYIETLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKTLLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMQRILKLTMQMRNSKTGTDTDYIVSPVADANGRFFDSRQADATMPKDADANGAYNIARKGIMLVQQIKQSDDLRTMKFDISNKSWLRFAQHTNQADE

SEQ ID NO: 51 - protein, 1238 aa, synthetic construct
MGKLEDFTNLYSLSKTLRFELRPIGKTRENIENGGLLRQDEDRAEKYVHIKKLIDEYHKAYIDKQLSGLVLQYADIGKANSLEEYYHSTRKSKDSDKDKIVKIQDNLRKQIVKRLKDSDEFKRIDKKELIQSDLAEFIKPAEDRALIAEFKNFTTYFTGFNENRQNMYSDKAISTAIAYRLIHENLPKFIDNIETFDRIAGITELYDQTSSDAEIFRLEHFSETLSQKQIDAYNSVMGRYNMLINEYNQTHKQSRLPKFKMLYKQILSDREHPSWLPEQFESDTAVLTAIRECYDDLRIPMANLKTLLEGLGNYDPSGIFLRNDQHLSQISKRLTGDRSSIERSVTEDLLTSRRLNKRKSRTTDEEESRKLFKQKGSLSIGYIADTAKIDVERYFAKLGAINTVTEQSENLFAKAENARTTADELLANDYPAGKRLVQSNDDIALLKNLLDALMELQWFVKPLLGTGDEAGKDERFYGEFAQIWEQLDRITPLYNMVRNYVTRKPYSTDKFKLNFESAALLGGWDKNKEPDCLSVILRKDEQYYLGIINKNHKKIFENDILPCEGECYDKMVYKLLPGANKMLPKVFFSASRIAEFAPSDEVKRIYNDKTFQKGEKFDLNDCRTLIDFYKASIDKHEEWNKFGFEFSDTNNYEDISGFFREVDRQGYKMSFRPVAASYIETLVEEGKLYLFQIYNKDFSAYSKGTPNMHTLYWRMLFDERNLSDVVYQLNGGAELFFRRKSLQNGRPTHPANIPIKNKNSRNDKKESLFDYDLIKDRRYTVDKFQFHVPITLNFKSDGAGRINERVREYLRSADDVHVIGIDRGERNLLYLVVTDMDGNICEQFSLNEICNTDYHSLLDEREHKRMQERQSWQAIEGIKELKEGYLSQVVHRIATLMVKYRAIVVLEDLNFGFMRSRQKVEKSVYQKFEHMLIDKLNYLVDKKANPTTPGGLLKAYQLTDKFESFQKLGKQSGFLFYVPAWNTSKIDPATGFVNMLDLGYESIDKAKTLLCKFDSIRYNACKDWFEFALDYDKFGSKATGTRTKWTVCTYGQRIDTYRNKDSQWVSRDVDLTNELKSLFSEHGIDIYSNLKDAIVAQNDKEFFANMQRILKLTMQMRNSKTGTDTDYIVSPVADANGRFFDSRQADATMPKDADANGAYNIARKGIMLVQQIKQSDDLRTMKFDISNKSWLRFAQHTNQADESRKRTADGSEFESPKKKRKVGSGKPIPNPLLGLDST

SEQ ID NO: 52 - DNA, 3606 bp, unidentified
  source 1..3606: mol_type = unassigned DNA
atgaaattagaagattttaccaacctgtattcgttatccaagactctgcgtttcgaactgcggccgatcggcaagacacgtgaaaatatcgaaaacggaggccttttgaggcaggacgaggatcgtgctgaaaaatatgtacacataaaaaaactaatcgatgaatatcataaagcatatatcgataaacaattgtcgggtttagtgctgcaatacgccgatatcggtaaagccaattcattggaggagtattatcactccacaagaaagagcaaagattcggacaaggataagattgtcaaaatccaggataatctgcgtaaacaaattgtcaaacggttgaaagactcagacgaattcaagcgtatcgataaaaaagagttgattcaatcggatctggcagagttcataaaaccagccgaagacagagctttgattgccgaattcaaaaacttcacaacatattttaccggattcaatgaaaacagacagaacatgtattcggacaaagctatatctacggcaatagcttatcgtctgatacatgagaatcttccgaagttcatagacaacatagagactttcgatcgcatcgccggtataacggaattgtacgaccaaacctcctccgatgccgaaattttccgtctggaacatttttcggaaacactgagccaaaagcagatcgatgcctataactccgttatgggcagatataacatgcttatcaatgagtacaatcagacgcataaacagtcgcgcctacctaaattcaaaatgctgtacaaacagattcttagcgaccgcgaacacccctcgtggctgcccgagcagttcgagtcggacacggctgtattgacagccattcgcgaatgttacgatgatctgcgcatacctatggccaatttgaaaacgcttttagaggggttgggcaactatgacccgagtggaatatttttgcgtaatgaccaacatctctctcagatatccaaacgattgacaggtgatcggagtagcattgaacgtagcgtaacagaagaccttctgacatcgaggagactcaacaagcgaaaaagccgcacaaccgacgaggaggaatcgagaaaactgttcaagcaaaagggtagtctgagtataggctatatagctgacacggccaaaatcgatgtcgaaagatactttgccaaactcggtgcaataaatacggtaacggagcagagcgagaatctattcgccaaggctgagaatgcccgcacgacagcggatgagctgctcgcaaatgattacccggcaggcaagaggctcgttcagtccaacgacgacatagcattgctgaaaaatctgctcgatgctttaatggagctgcaatggttcgtcaagccgctgcttggcacgggggacgaagccggcaaagacgaacgtttctatggagaatttgcacagatatgggagcagctggatcgtataacgcctctctataacatggtgcgcaactatgttacccgcaagccgtattcgaccgacaaattcaagctcaactttgagagcgcagcgcttctcggcggctgggacaagaacaaggagccggactgtctgtcggtaatcttacgcaaggatgagcaatattatctcggcataatcaataagaatcacaaaaagatattcgagaacgatatcttgccgtgcgaaggggagtgttacgacaaaatggtatataaactcctgcccggcgcaaacaagatgctgccgaaagtattcttctcggcttcgcgtatcgccgaatttgcaccgagcgacgaagtaaaacggatatacaatgataagactttccaaaaaggcgaaaagttcgacttgaacgattgtcgcacactgatcgacttctacaaggcttctatcgacaaacatgaggagtggaacaagtttggattcgaattctcggatacgaacaattatgaagacataagcggattctttcgcgaggtcgacaggcaaggctataaaatgtcattccgcccggtcgcagcatcgtatatcgaaacccttgttgaagagggcaaactctatcttttccaaatatataataaggatttttcggcatatagcaaaggtactcccaatatgcacacgctgtattggaggatgctcttcgacgagcgcaatctatcggatgtcgtatatcagctcaacggcggagcagagttgttcttccgaagaaagagtcttcaaaacggccgtccgacgcatccggcaaatattcctatcaaaaacaaaaacagtcggaatgacaaaaaagagagcctgttcgactacgatttgatcaaagacagacgctatactgtggacaaatttcagttccatgtcccgataaccctcaatttcaagagcgacggggcgggcaggatcaacgagcgtgtaagggaatatctccgctcggcggacgacgttcacgtcataggcatcgaccgcggagaacgcaatctgctgtatctggtcgtgacggatatggacggcaatatctgcgaacaattctcgctcaacgaaatttgtaatactgattatcattctttgttggatgaacgcgaacacaaacgtatgcaggagagacagagctggcaggcgatagagggcatcaaggagttgaaagaaggttatctgtctcaggtcgtacaccgaatcgcgacactcatggttaaatatcgcgccattgtcgtactggaagatctcaacttcggcttcatgcgtagccgccagaaggtagagaagtctgtataccagaaattcgaacacatgctcatagataagctcaattatctggtcgacaagaaagccaatccgacaacgccgggcggtctgctaaaagcctatcagttgacagacaaattcgagagcttccagaagctcggcaaacagagcggatttctattctacgttccggcatggaatacatcgaagatcgatccagcaaccggattcgtcaacatgctcgatctcggatacgagagcatcgacaaagccaaaacactgctctgcaagttcgactctatacgctacaatgcgtgcaaagactggttcgagttcgctctcgattacgacaagttcggcagcaaggccaccggtacccgcacgaaatggactgtttgcacctacggacaacgtatcgatacttatcgcaacaaagattcgcagtgggtcagccgcgacgtcgatttgacaaatgagctgaaatcactcttctccgaacacggcatagacatttacagcaatctgaaagatgcaatagtcgcacaaaacgacaaagaatttttcgcgaacatgcagcggatattgaaactgaccatgcaaatgcgaaacagcaaaacgggtaccgacacagactatatcgtctcgcccgtcgccgatgccaacggcagattcttcgacagcaggcaggccgatgcgaccatgcccaaagatgcggatgcgaacggagcgtataatatcgcacgtaagggcattatgctcgtacagcagatcaagcagtccgacgatctgcgtacaatgaagttcgacataagcaacaagagctggctgcgcttcgcccaacatacgaaccaggcggacgagtaa

SEQ ID NO: 53 - DNA, 3600 bp, synthetic construct
  source 1..3600: mol_type = other DNA
aagctggaggacttcaccaatctgtactctctgagcaagaccctgcggtttgagctgcggcctatcggcaagacaagagaaaacatcgaaaacggcggactgctgcgtcaagacgaggacagagccgaaaagtacgtgcatattaaaaagctgatcgatgaataccacaaggcttatatcgacaagcaactgagtggcctggtcctgcaatacgccgatatcggcaaggccaattctctggaggagtactaccacagcactagaaaaagcaaggactctgacaaggataagatagtcaagatccaggacaacctgcgcaagcagatcgtcaagagattgaaggacagcgatgagtttaagaggatcgataagaaggaactgatccagtctgacctggcagagttcatcaagccagccgaggacagggccctgatagccgagttcaagaacttcaccacctacttcacaggattcaacgaaaataggcagaacatgtacagcgataaggctatcagcaccgccatcgcctaccggctgatccacgagaacctgcctaagttcatcgacaacatcgaaaccttcgaccggatcgcgggcatcacagagctgtatgaccagacatccagcgacgcagagatctttagactggagcacttcagtgagacactgagccagaagcagatcgatgcctataacagcgtgatgggccggtacaacatgctgatcaacgaatataaccagacccacaagcaatctcggctgcctaaattcaaaatgctgtacaagcagatcctgagcgaccgggagcaccccagctggctgccggaacagttcgagagcgacaccgccgtgctgaccgccatcagagagtgttacgacgacctgagaatccctatggccaacttaaaaaccctgcttgagggcctgggaaattacgatccctctggcatcttcctgcggaacgatcagcacctgtctcagatcagcaaaagactcaccggagacagatccagcatcgaacggagcgtgaccgaggacttattaacgagccggagactgaacaaaagaaagagcagaaccaccgatgaagaggaaagcagaaagctgttcaagcaaaaaggcagcctgagcatcggctacatcgccgacacagccaagatcgacgtggagagatacttcgccaagctgggagccattaataccgtgaccgagcagtctgagaacctcttcgctaaggccgagaacgccagaaccactgctgacgagctgctggccaacgactaccctgccggcaaaagactggtgcagagcaacgacgacatcgctctgctgaagaacctattggacgccctgatggaactgcaatggttcgtgaagcccctgctgggcaccggcgacgaggccggcaaagacgaacggttctatggcgagttcgctcagatctgggagcagctggatagaatcacccctctgtacaacatggtgcggaattacgtgacaagaaagccctactccacagacaagttcaagctgaacttcgaatctgccgccctgctgggcggatgggacaagaacaaagaacctgactgcctgtccgtgattctgagaaaggacgagcagtactacctgggcatcatcaacaagaaccacaagaagatcttcgagaatgacattctgccttgcgagggcgagtgctacgacaagatggtctacaagctgctgcctggcgctaacaaaatgctgcctaaggtgttctttagcgcctccagaatcgctgagttcgccccttctgatgaggtgaaaagaatttacaacgataagaccttccagaagggcgagaagttcgatctgaacgactgcagaaccctcatcgatttctacaaggcttctatcgataagcacgaggagtggaataaatttggcttcgagtttagcgacaccaacaactacgaggacatcagcggcttcttccgggaggtggacagacagggctacaagatgagctttagacccgtggccgccagctacatcgaaacgttggtggaagagggcaaactgtacctgttccagatctacaacaaagatttcagcgcctacagcaagggcacccctaatatgcacaccctgtactggagaatgctgtttgacgagcggaacctgagcgacgtggtgtaccagctgaacggcggagctgaactgttctttagacgcaagtccctccagaacggccggcctacacaccctgccaacatccctatcaagaacaagaacagcagaaacgataaaaaggaatcactgttcgactacgatctcatcaaggatcgtagatacacagtggataagttccagttccacgtgccaatcacactgaatttcaagagcgatggcgctggcagaattaacgagagagtgcgggagtacctgagatctgccgatgacgtgcacgtgatcggcatcgacagaggcgagcggaacctgctgtacctcgtggtgaccgatatggacggcaacatctgcgaacagtttagcctgaacgaaatctgtaataccgactaccacagcctgttggatgagagagagcacaaaagaatgcaggaaagacagagctggcaggccatcgagggaatcaaggagctgaaggaaggctacctgtcccaagtggtccacagaatcgccaccctgatggtgaagtacagagcgatcgtggtgctggaggacctgaacttcggcttcatgcggagcagacagaaagtggaaaaaagcgtgtaccagaagttcgagcacatgctgatcgacaaactgaactacctggtggacaagaaagccaaccctaccacacccggcggcctgctgaaggcctaccagctgacagacaagttcgagagcttccagaagctgggcaagcagtctggattcctgttttatgtgcccgcctggaacacaagcaagatcgaccctgctaccggattcgtgaacatgctggatctgggctatgagagcatcgacaaggccaaaaccctgctgtgcaagtttgactccatcagatacaacgcctgcaaggactggttcgagtttgccctggactacgacaagttcggcagcaaggccacaggcacacggaccaagtggacagtgtgcacctacggccagcggatcgatacttatagaaacaaggacagccagtgggtgtctcgggacgtggatctgaccaatgagctgaagagcctgttttctgaacatggcatcgacatctacagcaacctgaaagacgccatcgtggcccaaaatgacaaagagttcttcgccaacatgcagagaatcctgaagctgaccatgcagatgagaaattctaaaactggaacagatacagactacattgtgtcccctgttgccgatgctaacggaagattcttcgacagcagacaagccgacgccaccatgccaaaggacgccgacgccaacggcgcctacaacatcgctagaaagggcatcatgctggttcagcagatcaagcagagcgatgacctccgcaccatgaaattcgacatcagcaacaagagctggctgagattcgcccagcataccaaccaggccgatgag

SEQ ID NO: 54 - DNA, 3717 bp, synthetic construct
  source 1..3717: mol_type = other DNA
atgggcaagctggaggacttcaccaatctgtactctctgagcaagaccctgcggtttgagctgcggcctatcggcaagacaagagaaaacatcgaaaacggcggactgctgcgtcaagacgaggacagagccgaaaagtacgtgcatattaaaaagctgatcgatgaataccacaaggcttatatcgacaagcaactgagtggcctggtcctgcaatacgccgatatcggcaaggccaattctctggaggagtactaccacagcactagaaaaagcaaggactctgacaaggataagatagtcaagatccaggacaacctgcgcaagcagatcgtcaagagattgaaggacagcgatgagtttaagaggatcgataagaaggaactgatccagtctgacctggcagagttcatcaagccagccgaggacagggccctgatagccgagttcaagaacttcaccacctacttcacaggattcaacgaaaataggcagaacatgtacagcgataaggctatcagcaccgccatcgcctaccggctgatccacgagaacctgcctaagttcatcgacaacatcgaaaccttcgaccggatcgcgggcatcacagagctgtatgaccagacatccagcgacgcagagatctttagactggagcacttcagtgagacactgagccagaagcagatcgatgcctataacagcgtgatgggccggtacaacatgctgatcaacgaatataaccagacccacaagcaatctcggctgcctaaattcaaaatgctgtacaagcagatcctgagcgaccgggagcaccccagctggctgccggaacagttcgagagcgacaccgccgtgctgaccgccatcagagagtgttacgacgacctgagaatccctatggccaacttaaaaaccctgcttgagggcctgggaaattacgatccctctggcatcttcctgcggaacgatcagcacctgtctcagatcagcaaaagactcaccggagacagatccagcatcgaacggagcgtgaccgaggacttattaacgagccggagactgaacaaaagaaagagcagaaccaccgatgaagaggaaagcagaaagctgttcaagcaaaaaggcagcctgagcatcggctacatcgccgacacagccaagatcgacgtggagagatacttcgccaagctgggagccattaataccgtgaccgagcagtctgagaacctcttcgctaaggccgagaacgccagaaccactgctgacgagctgctggccaacgactaccctgccggcaaaagactggtgcagagcaacgacgacatcgctctgctgaagaacctattggacgccctgatggaactgcaatggttcgtgaagcccctgctgggcaccggcgacgaggccggcaaagacgaacggttctatggcgagttcgctcagatctgggagcagctggatagaatcacccctctgtacaacatggtgcggaattacgtgacaagaaagccctactccacagacaagttcaagctgaacttcgaatctgccgccctgctgggcggatgggacaagaacaaagaacctgactgcctgtccgtgattctgagaaaggacgagcagtactacctgggcatcatcaacaagaaccacaagaagatcttcgagaatgacattctgccttgcgagggcgagtgctacgacaagatggtctacaagctgctgcctggcgctaacaaaatgctgcctaaggtgttctttagcgcctccagaatcgctgagttcgccccttctgatgaggtgaaaagaatttacaacgataagaccttccagaagggcgagaagttcgatctgaacgactgcagaaccctcatcgatttctacaaggcttctatcgataagcacgaggagtggaataaatttggcttcgagtttagcgacaccaacaactacgaggacatcagcggcttcttccgggaggtggacagacagggctacaagatgagctttagacccgtggccgccagctacatcgaaacgttggtggaagagggcaaactgtacctgttccagatctacaacaaagatttcagcgcctacagcaagggcacccctaatatgcacaccctgtactggagaatgctgtttgacgagcggaacctgagcgacgtggtgtaccagctgaacggcggagctgaactgttctttagacgcaagtccctccagaacggccggcctacacaccctgccaacatccctatcaagaacaagaacagcagaaacgataaaaaggaatcactgttcgactacgatctcatcaaggatcgtagatacacagtggataagttccagttccacgtgccaatcacactgaatttcaagagcgatggcgctggcagaattaacgagagagtgcgggagtacctgagatctgccgatgacgtgcacgtgatcggcatcgacagaggcgagcggaacctgctgtacctcgtggtgaccgatatggacggcaacatctgcgaacagtttagcctgaacgaaatctgtaataccgactaccacagcctgttggatgagagagagcacaaaagaatgcaggaaagacagagctggcaggccatcgagggaatcaaggagctgaaggaaggctacctgtcccaagtggtccacagaatcgccaccctgatggtgaagtacagagcgatcgtggtgctggaggacctgaacttcggcttcatgcggagcagacagaaagtggaaaaaagcgtgtaccagaagttcgagcacatgctgatcgacaaactgaactacctggtggacaagaaagccaaccctaccacacccggcggcctgctgaaggcctaccagctgacagacaagttcgagagcttccagaagctgggcaagcagtctggattcctgttttatgtgcccgcctggaacacaagcaagatcgaccctgctaccggattcgtgaacatgctggatctgggctatgagagcatcgacaaggccaaaaccctgctgtgcaagtttgactccatcagatacaacgcctgcaaggactggttcgagtttgccctggactacgacaagttcggcagcaaggccacaggcacacggaccaagtggacagtgtgcacctacggccagcggatcgatacttatagaaacaaggacagccagtgggtgtctcgggacgtggatctgaccaatgagctgaagagcctgttttctgaacatggcatcgacatctacagcaacctgaaagacgccatcgtggcccaaaatgacaaagagttcttcgccaacatgcagagaatcctgaagctgaccatgcagatgagaaattctaaaactggaacagatacagactacattgtgtcccctgttgccgatgctaacggaagattcttcgacagcagacaagccgacgccaccatgccaaaggacgccgacgccaacggcgcctacaacatcgctagaaagggcatcatgctggttcagcagatcaagcagagcgatgacctccgcaccatgaaattcgacatcagcaacaagagctggctgagattcgcccagcataccaaccaggccgatgagtctagaaagcggacagcagacggctccgaatttgaaagccctaagaaaaagagaaaggtgggatccggcaaacctatccccaatcccctgctgggcctggacagcacctga

SEQ ID NO: 55 - protein, 1264 aa, unidentified
MKDLTGQYSLSKTLRFELKPIGKTLEHIEQKGLLTQDEQRAEEYEQMKGIIDRYHKAFITMCLRNCKIKVNNTDDELDSLEEYSSLLSKSKRDADDENKLEKIKENLRKQIVNAFKSGNTYGDLFTKELIKNHLPDFVTDEEEKQVVEHFCNFTTYFTGFHDNRKNMYSDKAKSTAIAYRLIHENFPRFFDNLRSFAKISESEVANRFPEIESAFSLYLNVEHIADMFHVDYFPVVLTQEQIDVYNNIIGGKTEEDGTKIQGINEYINLYNQHHPDVKLPFLKPLYKMILSDKVALSWLPEEFENDEEMLTAINDFYKSVQPVVFGDDENCIRHLLTNIAEYNTDHIYISNDLGLTGISQQLFDQYSIFEDVIKDELRRNVKQTPKEKRNPELLEERIKNLFKKEKSFSISYLDSLIKDKGEDTIESYYAKLGAFDRDGKQTVNLLTQIEMAYIAAKEVLDGKYDNINQSEEATKYIKDLLDAFKSLQHYIKPLLGSGEEAEKDNVFSSQLLNVWEALDVVTPLYNKVRNWLTRKPYSTKKIKLNFENVQLLGGWPNIEAYSCAIFMKDDNTYYLGILDNAYKTLLRDFPEPAEEKDTIGLMHYLQGGDMGKNIQNLMVVDGKVRKVNGRKEKSGINVGQNIRLEEAKKRYLPTEINRIRKLGTYSVSNPNYNKQDLITIIDYYKPLACEYYASYTFHFKDSSEYNSFAEFTDDINQQAYQLGFVPFSQQYLNKLVDEGKLYLFQIWNKDFSDYSKGTPNMHTLYWKALFDKANLADVVYKLNGRQAEVFYRKRSLQKENTTVHKALQPIKNKNTQNEKSTSTFDYDIVKDRRYTVDKFHFHVPITINFKSSGKPNINEHVLDIIRHHGIEHVIGIDRGERHLLYLSLIDLKGRIIKQMTLNEIKQQTGGNYGTNYKELLAAREGDRAEARRNWKKIENIKDLKAGYLSQVVHVIAQMMVEYNAIVVLEDLNMGFMRGRQKIERSVYEQFEHMLIDKLNFYVDKKKEACAPGGLLHGLQLANKFESFNKLGKQSGCLFYVPAWNTSKIDPVTGFVNMLDARYESVESSRRFFSRFDVIRYNEEKNWFEFTFDYNNFHAKLDGTKTQWTLCTYGSRIKTFRNPAKLNQWDNEEVVLTDEFKKVFANAGINIHGNLKEAICSLAKREHLEPLMHLMKLLLQLRNSKTNSEVDYMLSPVADNGVFYDSRSCNGNLPIDADANGAYNIARKGLWVLRQIQDSKPGDKLNLALSNKEWLRFVQEKSNFE

SEQ ID NO: 56 - protein, 1264 aa, unidentified; identical to SEQ ID NO: 55

SEQ ID NO: 57 - protein, 1301 aa, synthetic construct
MGKDLTGQYSLSKTLRFELKPIGKTLEHIEQKGLLTQDEQRAEEYEQMKGIIDRYHKAFITMCLRNCKIKVNNTDDELDSLEEYSSLLSKSKRDADDENKLEKIKENLRKQIVNAFKSGNTYGDLFTKELIKNHLPDFVTDEEEKQVVEHFCNFTTYFTGFHDNRKNMYSDKAKSTAIAYRLIHENFPRFFDNLRSFAKISESEVANRFPEIESAFSLYLNVEHIADMFHVDYFPVVLTQEQIDVYNNIIGGKTEEDGTKIQGINEYINLYNQHHPDVKLPFLKPLYKMILSDKVALSWLPEEFENDEEMLTAINDFYKSVQPVVFGDDENCIRHLLTNIAEYNTDHIYISNDLGLTGISQQLFDQYSIFEDVIKDELRRNVKQTPKEKRNPELLEERIKNLFKKEKSFSISYLDSLIKDKGEDTIESYYAKLGAFDRDGKQTVNLLTQIEMAYIAAKEVLDGKYDNINQSEEATKYIKDLLDAFKSLQHYIKPLLGSGEEAEKDNVFSSQLLNVWEALDVVTPLYNKVRNWLTRKPYSTKKIKLNFENVQLLGGWPNIEAYSCAIFMKDDNTYYLGILDNAYKTLLRDFPEPAEEKDTIGLMHYLQGGDMGKNIQNLMVVDGKVRKVNGRKEKSGINVGQNIRLEEAKKRYLPTEINRIRKLGTYSVSNPNYNKQDLITIIDYYKPLACEYYASYTFHFKDSSEYNSFAEFTDDINQQAYQLGFVPFSQQYLNKLVDEGKLYLFQIWNKDFSDYSKGTPNMHTLYWKALFDKANLADVVYKLNGRQAEVFYRKRSLQKENTTVHKALQPIKNKNTQNEKSTSTFDYDIVKDRRYTVDKFHFHVPITINFKSSGKPNINEHVLDIIRHHGIEHVIGIDRGERHLLYLSLIDLKGRIIKQMTLNEIKQQTGGNYGTNYKELLAAREGDRAEARRNWKKIENIKDLKAGYLSQVVHVIAQMMVEYNAIVVLEDLNMGFMRGRQKIERSVYEQFEHMLIDKLNFYVDKKKEACAPGGLLHGLQLANKFESFNKLGKQSGCLFYVPAWNTSKIDPVTGFVNMLDARYESVESSRRFFSRFDVIRYNEEKNWFEFTFDYNNFHAKLDGTKTQWTLCTYGSRIKTFRNPAKLNQWDNEEVVLTDEFKKVFANAGINIHGNLKEAICSLAKREHLEPLMHLMKLLLQLRNSKTNSEVDYMLSPVADNGVFYDSRSCNGNLPIDADANGAYNIARKGLWVLRQIQDSKPGDKLNLALSNKEWLRFVQEKSNFESRKRTADGSEFESPKKKRKVGSGKPIPNPLLGLDST

SEQ ID NO: 58 - DNA, 3795 bp, unidentified
  source 1..3795: mol_type = unassigned DNA
atgaaagacctgacagggcaatatagcctgtcgaaaactttacgatttgagttaaaacctatcggtaaaactcttgagcacattgagcaaaaaggactcttgacacaggacgaacaaagagcagaagagtacgagcaaatgaaaggtatcatcgaccgatatcacaaggcatttattaccatgtgtttgagaaactgcaaaatcaaggtaaataatacagacgacgaattagactcattagaagaatactcctcattactttccaaaagtaaaagagatgctgatgatgagaacaaattggaaaagattaaggaaaatcttcgcaagcaaatcgtcaatgctttcaaaagcggcaacacttatggcgacttgttcacaaaggaactgattaagaatcatctgcccgacttcgtcacagacgaggaagaaaagcaagtggtggagcatttctgcaattttaccacatattttacgggtttccacgacaaccgcaaaaacatgtactcagataaggctaaatccacggcaatagcctatcgcctgatacatgagaatttccctcggttttttgacaatcttcgctcttttgcaaagatttcagaaagcgaggtggcaaatcggttccctgagatagaatctgctttctctctgtatctcaacgtggaacacatcgccgacatgttccacgttgactatttcccagttgttcttacccaagaacaaattgatgtgtataataatattattggaggcaagacggaagaagatgggacaaaaatacagggcatcaatgaatacatcaacctttataaccaacatcacccagatgtaaagttgccgttcttgaaacctctatacaagatgattcttagcgacaaggttgcgctttcatggttgccggaggagtttgagaatgatgaagagatgttgacggccataaatgatttttacaagtcagttcagcctgtcgttttcggggatgacgagaattgtatccgtcatcttctgacgaatattgccgaatacaatacggatcacatatacatttcaaacgatttaggattgactggaatatcccagcaattgttcgaccaatacagcatctttgaagacgtcattaaagatgagttgaggcgtaatgtcaaacagacgcccaaagagaaacgcaatcctgaattgttggaagaaagaataaagaacttgttcaagaaagagaagagtttctccatctcttacctggactctctcattaaggataagggtgaggatacgatcgagtcttattatgccaaacttggtgcgtttgacagagacggtaagcaaacagtgaatttgctcacgcaaattgaaatggcatacatagcggcaaaggaggtgcttgatggtaagtatgacaacattaaccagtctgaagaagcaacgaaatatattaaagatcttcttgatgcgttcaagtctttgcaacactacatcaaaccgctgttaggtagtggcgaagaagcagaaaaggataatgtgtttagttcgcaactgctcaatgtttgggaggcgttagacgttgtgactcctctttataacaaagttcgcaactggctcacacgcaagccttactcaacaaaaaagataaagctgaactttgagaatgtccaactgcttggcggctggccaaatatagaagcgtattcatgtgctatttttatgaaggatgataatacttactatcttggaatactggacaatgcatataaaactttattaagagattttccagagcctgccgaagagaaggatactattgggctaatgcattacctccaaggaggcgatatgggaaaaaatattcagaatttgatggtggtagatggaaaggttcggaaagttaatgggcgcaaagagaagtcaggaattaatgttgggcagaatattcgattagaagaagcaaaaaagagatacctgccaacagaaatcaatagaataaggaagttgggaacgtattctgtttcaaatccaaattataacaaacaagatttgataaccataatcgattattacaagccactggcttgtgaatactatgcttcctatacattccatttcaaggattcttccgagtataattcgttcgcggagtttacagacgatatcaatcagcaagcgtatcaacttgggtttgtacctttttctcaacaatacttaaacaaacttgtagacgaaggcaaactctaccttttccaaatatggaataaagatttctctgattatagtaaaggcactcccaatatgcataccctttattggaaggcgctctttgataaagcaaatcttgccgatgttgtctacaaacttaatggtcgtcaggcagaggtgttctatcggaaaagaagcctccaaaaagagaatacgactgtgcacaaagcattgcagcctataaagaataaaaacacgcagaatgagaaaagcaccagtacgtttgactatgacatcgtaaaagatcgtcgttatacagttgataaattccatttccatgtgcccattactattaactttaagtcatctggaaaacctaatatcaatgaacacgttttagatattatccgtcaccatggcattgagcatgtcatcggaatcgaccgtggcgagcgccatctattatatctttctcttatagatctcaagggaagaataatcaagcaaatgacgcttaatgagataaagcagcaaacaggcggtaactatggcacaaattataaagaactcttggccgcaagagaaggcgatcgtgcggaagcgcgtcgtaactggaaaaagatagagaatattaaagaccttaaagctggctatctcagtcaggttgtacatgtgatagcccaaatgatggtggaatacaatgccatcgttgtgctcgaagacctcaatatgggctttatgcgtgggcggcagaaaatcgagcggagcgtatacgagcagttcgaacacatgctgatagataagttgaacttctatgttgataagaaaaaggaagcatgtgcccccggaggtctgcttcatggtctccaattagccaataaatttgagagcttcaataagcttgggaaacagagcggttgccttttttatgtaccggcatggaataccagcaaaatagatcctgtcacagggtttgtcaatatgcttgatgcacgctatgaaagtgtagaaagttcgcgccgcttcttctctcgtttcgatgttattcgttacaatgaggaaaagaattggtttgaatttacttttgattataataacttccatgcaaagttggacgggacaaaaacccaatggacgctttgcacatacggcagtcgcatcaaaacattccgcaaccccgcaaaactcaatcaatgggataatgaagaggtggttcttaccgatgaatttaagaaggtatttgccaatgctggtatcaatattcatgggaatttgaaagaggccatttgctctcttgctaaacgggagcatttagaaccgttgatgcatttgatgaaactgcttttacagttgcgcaacagcaagaccaactcagaggtcgactatatgctttctcctgtggcagataatggcgtgttttacgacagccgttcttgcaatggcaatttgcctatagatgccgatgccaatggggcatacaacattgcccggaaaggattatgggttttgcgccaaattcaggactctaagcctggcgacaaactgaatttggctttgtcgaacaaggaatggttgcgatttgttcaagaaaagagcaactttgaataa

SEQ ID NO: 59 - DNA, 3789 bp, synthetic construct
  source 1..3789: mol_type = other DNA
aaggatctgacaggccagtacagcctctctaagaccctcagatttgaactgaagcctatcggcaagaccctggagcacatcgagcaaaagggcctgctgacccaggacgagcagagagccgaggaatacgagcagatgaagggaattattgacagataccacaaggccttcatcactatgtgcctgagaaattgcaagatcaaggtgaacaacaccgacgatgagctggacagcctggaagagtacagcagcctgctgtcaaagtctaagcgggacgccgacgacgagaacaaactggagaagatcaaggaaaacctgagaaagcagatcgtcaatgccttcaagagcggaaacacctacggcgatctgttcaccaaggagctgatcaagaaccacctccccgattttgtgaccgacgaggaagaaaagcaggtggtggaacacttctgcaacttcaccacctacttcaccggctttcacgacaaccgcaagaacatgtacagcgacaaggccaagagcacagccatcgcctacagactgatccacgagaactttccaagatttttcgataatctgcggagctttgccaagatctccgaatctgaagtggccaacagattcccagaaatcgagagcgcctttagcctgtacctgaatgtggaacatatcgccgatatgttccacgtggactacttcccagtggtgctgacccaggagcagattgacgtgtacaacaacatcatcggaggcaagaccgaggaagatggcacaaagattcagggcatcaacgagtatatcaacctgtacaaccaacaccatcctgacgtcaaactgcccttcctgaagcctctgtataagatgatcctgagcgacaaggtggccctgagctggctgcctgaagagttcgagaacgacgaggaaatgctgaccgccatcaatgatttctacaagtctgtgcagcctgtggtgttcggcgatgacgagaactgtatcagacacctgctgacaaacatcgccgagtacaacaccgatcacatttacatcagcaatgacctgggactgactggcatctctcagcagctgttcgaccagtactctatcttcgaagatgtgatcaaggacgagctacggcggaacgtgaagcaaacacctaaggagaagcggaaccccgaactgctggaagagagaatcaagaacctgttcaagaaagaaaagagcttctccatcagctacctggatagcctgatcaaggacaaaggagaagataccatcgagagctactacgccaagctgggcgccttcgacagagatggcaagcagacagtgaacctgctcacccagatcgagatggcctacatcgccgctaaggaagtgctggatggcaagtacgacaacatcaaccagagcgaggaagctacaaagtacatcaaggatctgcttgacgccttcaagagcctgcagcactacatcaagcccctgctgggcagcggcgaggaggccgaaaaagacaacgtgttcagcagccagctcctgaacgtgtgggaggctctggacgtggtgacgcctctgtacaacaaggtcagaaattggctgacaagaaagccctacagtaccaagaaaatcaaactgaacttcgagaatgttcaactgctgggcggatggcctaacatcgaggcctatagctgcgccatttttatgaaagacgacaacacctactacttaggcatcctggacaacgcctataaaacactacttcgggactttcctgaacctgctgaagaaaaggacacaatcggcctgatgcactacctgcaaggaggcgacatgggcaagaacatccagaacctgatggtcgtcgacgggaaggtgcggaaggtgaacggccgtaaggaaaagtccggcatcaacgtgggccagaatatccggctggaggaggccaagaagagatacctgcctacagagatcaacagaatcagaaagctgggcacctactctgtgagcaaccctaattataacaagcaggatctgattacaatcatcgactactacaagccactggcctgcgagtactacgcctcttatacattccacttcaaggacagcagcgagtacaacagcttcgccgagttcaccgatgatatcaaccagcaggcctaccagttgggcttcgtgcctttctcccagcaatacctcaacaaactggtggacgagggcaagctgtacctgttccagatctggaataaggacttctctgactactctaagggcacccccaacatgcacaccctgtactggaaggccctgtttgacaaggccaatctggctgatgtggtttacaagctgaacggcagacaggccgaggtgttttacagaaagagaagcctgcagaaagagaacacaaccgtgcacaaggctctgcagcccatcaagaataagaacacacagaacgagaaatctaccagcacattcgattacgatatcgtgaaggacagaagatacaccgtggacaagttccatttccacgttcctatcaccatcaacttcaagtccagcggcaagcctaacatcaacgagcatgtgctggatatcatcagacaccacggcatcgagcacgtgatcggcatcgaccgcggcgaaaggcacctgctgtacctgtccctgatcgacctgaaaggacggatcataaagcagatgacccttaacgagatcaaacaacagaccggcggcaactacggcacaaactacaaagagctgctggccgccagagaaggcgacagagccgaggctagaagaaactggaagaaaatcgagaacatcaaggacctgaaggccggctacctgagccaggtggtgcacgtgattgctcagatgatggtggaatacaacgccattgtagtgctggaggacctgaacatgggcttcatgagaggcagacagaagatcgagagaagcgtgtacgagcagttcgagcacatgctgattgacaagctgaacttctacgtggacaaaaagaaggaagcatgcgcccctggcggacttctgcacggcctgcagctggccaacaaattcgagtctttcaacaaactgggcaagcaatccggctgtctgttctacgtgcccgcctggaacaccagcaagatcgatcctgtgaccggattcgtgaacatgctggacgcccggtacgagagcgtggagagctcccggcggttcttctccagatttgacgtgatcagatacaacgaggagaagaactggttcgagttcacctttgattataacaacttccacgccaaactggatggcaccaagacccagtggacactgtgcacctacggcagcagaatcaagacctttagaaatcctgctaagctgaatcagtgggacaatgaagaggtggttctgaccgacgaatttaagaaggtgttcgccaacgccggaatcaatatccacggcaacctgaaggaagctatctgcagcctggccaaaagagagcacctggaacctctgatgcacctgatgaaactgctgctgcaacttcggaatagcaaaaccaacagcgaggtcgactacatgctgtctccagtggccgataatggagtgttctacgacagcagaagctgtaacggtaacctgcctatcgacgccgacgccaacggagcctacaatatcgctagaaaaggtctgtgggtcctcaggcaaatccaggatagcaagcccggcgacaagctgaacctggctctgagcaacaaggaatggctgcgatttgtacaggagaaaagcaatttcgag

SEQ ID NO: 60 - DNA, 3906 bp, synthetic construct
  source 1..3906: mol_type = other DNA
atgggcaaggatctgacaggccagtacagcctctctaagaccctcagatttgaactgaagcctatcggcaagaccctggagcacatcgagcaaaagggcctgctgacccaggacgagcagagagccgaggaatacgagcagatgaagggaattattgacagataccacaaggccttcatcactatgtgcctgagaaattgcaagatcaaggtgaacaacaccgacgatgagctggacagcctggaagagtacagcagcctgctgtcaaagtctaagcgggacgccgacgacgagaacaaactggagaagatcaaggaaaacctgagaaagcagatcgtcaatgccttcaagagcggaaacacctacggcgatctgttcaccaaggagctgatcaagaaccacctccccgattttgtgaccgacgaggaagaaaagcaggtggtggaacacttctgcaacttcaccacctacttcaccggctttcacgacaaccgcaagaacatgtacagcgacaaggccaagagcacagccatcgcctacagactgatccacgagaactttccaagatttttcgataatctgcggagctttgccaagatctccgaatctgaagtggccaacagattcccagaaatcgagagcgcctttagcctgtacctgaatgtggaacatatcgccgatatgttccacgtggactacttcccagtggtgctgacccaggagcagattgacgtgtacaacaacatcatcggaggcaagaccgaggaagatggcacaaagattcagggcatcaacgagtatatcaacctgtacaaccaacaccatcctgacgtcaaactgcccttcctgaagcctctgtataagatgatcctgagcgacaaggtggccctgagctggctgcctgaagagttcgagaacgacgaggaaatgctgaccgccatcaatgatttctacaagtctgtgcagcctgtggtgttcggcgatgacgagaactgtatcagacacctgctgacaaacatcgccgagtacaacaccgatcacatttacatcagcaatgacctgggactgactggcatctctcagcagctgttcgaccagtactctatcttcgaagatgtgatcaaggacgagctacggcggaacgtgaagcaaacacctaaggagaagcggaaccccgaactgctggaagagagaatcaagaacctgttcaagaaagaaaagagcttctccatcagctacctggatagcctgatcaaggacaaaggagaagataccatcgagagctactacgccaagctgggcgccttcgacagagatggcaagcagacagtgaacctgctcacccagatcgagatggcctacatcgccgctaaggaagtgctggatggcaagtacgacaacatcaaccagagcgaggaagctacaaagtacatcaaggatctgcttgacgccttcaagagcctgcagcactacatcaagcccctgctgggcagcggcgaggaggccgaaaaagacaacgtgttcagcagccagctcctgaacgtgtgggaggctctggacgtggtgacgcctctgtacaacaaggtcagaaattggctgacaagaaagccctacagtaccaagaaaatcaaactgaacttcgagaatgttcaactgctgggcggatggcctaacatcgaggcctatagctgcgccatttttatgaaagacgacaacacctactacttaggcatcctggacaacgcctataaaacactacttcgggactttcctgaacctgctgaagaaaaggacacaatcggcctgatgcactacctgcaaggaggcgacatgggcaagaacatccagaacctgatggtcgtcgacgggaaggtgcggaaggtgaacggccgtaaggaaaagtccggcatcaacgtgggccagaatatccggctggaggaggccaagaagagatacctgcctacagagatcaacagaatcagaaagctgggcacctactctgtgagcaaccctaattataacaagcaggatctgattacaatcatcgactactacaagccactggcctgcgagtactacgcctcttatacattccacttcaaggacagcagcgagtacaacagcttcgccgagttcaccgatgatatcaaccagcaggcctaccagttgggcttcgtgcctttctcccagcaatacctcaacaaactggtggacgagggcaagctgtacctgttccagatctggaataaggacttctctgactactctaagggcacccccaacatgcacaccctgtactggaaggccctgtttgacaaggccaatctggctgatgtggtttacaagctgaacggcagacaggccgaggtgttttacagaaagagaagcctgcagaaagagaacacaaccgtgcacaaggctctgcagcccatcaagaataagaacacacagaacgagaaatctaccagcacattcgattacgatatcgtgaaggacagaagatacaccgtggacaagttccatttccacgttcctatcaccatcaacttcaagtccagcggcaagcctaacatcaacgagcatgtgctggatatcatcagacaccacggcatcgagcacgtgatcggcatcgaccgcggcgaaaggcacctgctgtacctgtccctgatcgacctgaaaggacggatcataaagcagatgacccttaacgagatcaaacaacagaccggcggcaactacggcacaaactacaaagagctgctggccgccagagaaggcgacagagccgaggctagaagaaactggaagaaaatcgagaacatcaaggacctgaaggccggctacctgagccaggtggtgcacgtgattgctcagatgatggtggaatacaacgccattgtagtgctggaggacctgaacatgggcttcatgagaggcagacagaagatcgagagaagcgtgtacgagcagttcgagcacatgctgattgacaagctgaacttctacgtggacaaaaagaaggaagcatgcgcccctggcggacttctgcacggcctgcagctggccaacaaattcgagtctttcaacaaactgggcaagcaatccggctgtctgttctacgtgcccgcctggaacaccagcaagatcgatcctgtgaccggattcgtgaacatgctggacgcccggtacgagagcgtggagagctcccggcggttcttctccagatttgacgtgatcagatacaacgaggagaagaactggttcgagttcacctttgattataacaacttccacgccaaactggatggcaccaagacccagtggacactgtgcacctacggcagcagaatcaagacctttagaaatcctgctaagctgaatcagtgggacaatgaagaggtggttctgaccgacgaatttaagaaggtgttcgccaacgccggaatcaatatccacggcaacctgaaggaagctatctgcagcctggccaaaagagagcacctggaacctctgatgcacctgatgaaactgctgctgcaacttcggaatagcaaaaccaacagcgaggtcgactacatgctgtctccagtggccgataatggagtgttctacgacagcagaagctgtaacggtaacctgcctatcgacgccgacgccaacggagcctacaatatcgctagaaaaggtctgtgggtcctcaggcaaatccaggatagcaagcccggcgacaagctgaacctggctctgagcaacaaggaatggctgcgatttgtacaggagaaaagcaatttcgagtctagaaagcggacagcagacggctccgaatttgaaagccctaagaaaaagagaaaggtgggatccggcaaacctatccccaatcccctgctgggcctggacagcacctga

SEQ ID NO: 61 - protein, 1261 aa, unidentified
TNTSIFKTFTNQYSLSKTLRFELRPHPMTSGLDDIISLDTGIKKLYENEMKPLFDELHFEFISQSLVQVSFPSEKLEVLLNKYRSLKDQKAKNIEKELEGPLQELRTIITDTFESTGNNWKKEWLQQGFKIKSSGYKVLTEEGILEVLSVRKKDKADAINKFKGFFTYFSGFNMNRENYYSSEDKKTAVAYRVINENLIRYMDNILLLQNVLAKAPEFKKFEDILSLTTFGKYINQEGITTYNNNVVATINLELNTYHQHNPKIFSRLPKLKLLYKQIGSPKEDKRIFTIEKRTEWQSLEDLIQKQNKVVEHEKKNVEILSNLKAAYISFFTNTDETILSEVYFNKRSLNTISSFWFTGGWQTLLLKLKEFKLANQNKDGDIVVPKALSLAELKQVLDSLEEQDPAVNHLFKEMYSDCYKENLWQTFIAIWQCEITSKFNLLEGYIQECNAVKEDTFDKKKHKNIIKNICDTYLDIEQISKYIIVHESLPKYDALYDAVILYLQESSLRSYYDAFRNLISKRPVNEEKVKLNFQNSTLLDGWDMNKESANLCVLLKNNIGEYFLAVMNKKSNMVFDQKKNSALYSAGNESSFQKLEYKLLPGPNKMLPKVIFAKSNEKYFIIPEEIVQIREEESFKKGKKFDKHALKTWIRFMQESIEKYPGWKTFDFTFKKPEEYEDVSKFYKDVEEQGYKLNWKDINEEELLSLVEQKKVYLFQIKSKDIGETKEHGNKNLHTLLFLELLKPENTSRLKLLGGGEMFYRAPSMEKVYKTVNEKQVLDSKGNPILEAKRYYEPKFFLHFPIQVKGSENGYKTEMNPKILRAISTSKEVNIIGIDRGEKHLLYYYVIKPDGTPITQGSLNTISLGLDKNQNPRLVDERTFKILERDSKGKPSKISDFESTGKKVDYIDYHNILTYYETKRNIARRSWDTIGAIKNFKEGYLSQAIHQIYQLMLKYNAVVVLEDLNTEFKAKRTAKVEKSVYEKFEIALAKKLNHLIIKGTDPAEAGSVINPYQLTPAITADTLSDFKKSKQWGPLFYIRANYTSTTDPITGWRKHIYIPSGASDKEIKTYFCKQGEKEPLIQISYDTALTAFAFTYTHEGKEWTLHATKDTQRMRYDSKKRKMEPVEIFDRLRELFIDFSFEESLTDQLEATLSFDWKTLAFLWTMLNQIRNTDREAEGNDGDFIQSPVAPFYDSRDPENKTNGLPVNGDANGAFNIARKGAILIKRIQEYAKKDPTFEKMREKDGLNLYISDAEWDTEIS

SEQ ID NO: 62 - protein, 1262 aa, unidentified
MTNTSIFKTFTNQYSLSKTLRFELRPHPMTSGLDDIISLDTGIKKLYENEMKPLFDELHFEFISQSLVQVSFPSEKLEVLLNKYRSLKDQKAKNIEKELEGPLQELRTIITDTFESTGNNWKKEWLQQGFKIKSSGYKVLTEEGILEVLSVRKKDKADAINKFKGFFTYFSGFNMNRENYYSSEDKKTAVAYRVINENLIRYMDNILLLQNVLAKAPEFKKFEDILSLTTFGKYINQEGITTYNNNVVATINLELNTYHQHNPKIFSRLPKLKLLYKQIGSPKEDKRIFTIEKRTEWQSLEDLIQKQNKVVEHEKKNVEILSNLKAAYISFFTNTDETILSEVYFNKRSLNTISSFWFTGGWQTLLLKLKEFKLANQNKDGDIVVPKALSLAELKQVLDSLEEQDPAVNHLFKEMYSDCYKENLWQTFIAIWQCEITSKFNLLEGYIQECNAVKEDTFDKKKHKNIIKNICDTYLDIEQISKYIIVHESLPKYDALYDAVILYLQESSLRSYYDAFRNLISKRPVNEEKVKLNFQNSTLLDGWDMNKESANLCVLLKNNIGEYFLAVMNKKSNMVFDQKKNSALYSAGNESSFQKLEYKLLPGPNKMLPKVIFAKSNEKYFIIPEEIVQIREEESFKKGKKFDKHALKTWIRFMQESIEKYPGWKTFDFTFKKPEEYEDVSKFYKDVEEQGYKLNWKDINEEELLSLVEQKKVYLFQIKSKDIGETKEHGNKNLHTLLFLELLKPENTSRLKLLGGGEMFYRAPSMEKVYKTVNEKQVLDSKGNPILEAKRYYEPKFFLHFPIQVKGSENGYKTEMNPKILRAISTSKEVNIIGIDRGEKHLLYYYVIKPDGTPITQGSLNTISLGLDKNQNPRLVDERTFKILERDSKGKPSKISDFESTGKKVDYIDYHNILTYYETKRNIARRSWDTIGAIKNFKEGYLSQAIHQIYQLMLKYNAVVVLEDLNTEFKAKRTAKVEKSVYEKFEIALAKKLNHLIIKGTDPAEAGSVINPYQLTPAITADTLSDFKKSKQWGPLFYIRANYTSTTDPITGWRKHIYIPSGASDKEIKTYFCKQGEKEPLIQISYDTALTAFAFTYTHEGKEWTLHATKDTQRMRYDSKKRKMEPVEIFDRLRELFIDFSFEESLTDQLEATLSFDWKTLAFLWTMLNQIRNTDREAEGNDGDFIQSPVAPFYDSRDPENKTNGLPVNGDANGAFNIARKGAILIKRIQEYAKKDPTFEKMREKDGLNLYISDAEWDTEIS

SEQ ID NO: 63 - protein, 1301 aa, synthetic construct
MGSGTNTSIFKTFTNQYSLSKTLRFELRPHPMTSGLDDIISLDTGIKKLYENEMKPLFDELHFEFISQSLVQVSFPSEKLEVLLNKYRSLKDQKAKNIEKELEGPLQELRTIITDTFESTGNNWKKEWLQQGFKIKSSGYKVLTEEGILEVLSVRKKDKADAINKFKGFFTYFSGFNMNRENYYSSEDKKTAVAYRVINENLIRYMDNILLLQNVLAKAPEFKKFEDILSLTTFGKYINQEGITTYNNNVVATINLELNTYHQHNPKIFSRLPKLKLLYKQIGSPKEDKRIFTIEKRTEWQSLEDLIQKQNKVVEHEKKNVEILSNLKAAYISFFTNTDETILSEVYFNKRSLNTISSFWFTGGWQTLLLKLKEFKLANQNKDGDIVVPKALSLAELKQVLDSLEEQDPAVNHLFKEMYSDCYKENLWQTFIAIWQCEITSKFNLLEGYIQECNAVKEDTFDKKKHKNIIKNICDTYLDIEQISKYIIVHESLPKYDALYDAVILYLQESSLRSYYDAFRNLISKRPVNEEKVKLNFQNSTLLDGWDMNKESANLCVLLKNNIGEYFLAVMNKKSNMVFDQKKNSALYSAGNESSFQKLEYKLLPGPNKMLPKVIFAKSNEKYFIIPEEIVQIREEESFKKGKKFDKHALKTWIRFMQESIEKYPGWKTFDFTFKKPEEYEDVSKFYKDVEEQGYKLNWKDINEEELLSLVEQKKVYLFQIKSKDIGETKEHGNKNLHTLLFLELLKPENTSRLKLLGGGEMFYRAPSMEKVYKTVNEKQVLDSKGNPILEAKRYYEPKFFLHFPIQVKGSENGYKTEMNPKILRAISTSKEVNIIGIDRGEKHLLYYYVIKPDGTPITQGSLNTISLGLDKNQNPRLVDERTFKILERDSKGKPSKISDFESTGKKVDYIDYHNILTYYETKRNIARRSWDTIGAIKNFKEGYLSQAIHQIYQLMLKYNAVVVLEDLNTEFKAKRTAKVEKSVYEKFEIALAKKLNHLIIKGTDPAEAGSVINPYQLTPAITADTLSDFKKSKQWGPLFYIRANYTSTTDPITGWRKHIYIPSGASDKEIKTYFCKQGEKEPLIQISYDTALTAFAFTYTHEGKEWTLHATKDTQRMRYDSKKRKMEPVEIFDRLRELFIDFSFEESLTDQLEATLSFDWKTLAFLWTMLNQIRNTDREAEGNDGDFIQSPVAPFYDSRDPENKTNGLPVNGDANGAFNIARKGAILIKRIQEYAKKDPTFEKMREKDGLNLYISDAEWDTEISSRKRTADGSEFESPKKKRKVGSGKPIPNPLLGLDST

SEQ ID NO: 64 - DNA, 3789 bp, unidentified
  source 1..3789: mol_type = unassigned DNA
atgactaacacatctattttcaaaaccttcactaatcaatattcactttcaaaaacgttgcggtttgagttgagacctcatccgatgactagtggtctagatgatatcatttcattagatactggcataaaaaaattgtatgaaaacgagatgaagccgctatttgatgaacttcattttgaatttatctctcagtcgctagttcaagtatcattcccttcagaaaaactggaagttttgctaaacaagtataggtctcttaaggatcagaaagctaaaaatatagaaaaagaactggaaggcccattacaggaactaagaacaattattactgacacctttgaatccactggtaacaactggaaaaaagaatggctacaacaagggtttaaaatcaaaagctcgggatacaaagtactaacagaagagggaatattagaagtattgtctgttcgtaaaaaagataaagcggatgcaatcaataaatttaaaggattcttcacgtacttttcagggtttaacatgaaccgtgaaaattattattcatcggaagataaaaaaacagctgtagcgtatagggtaattaatgaaaaccttatccggtatatggataacattctcctccttcagaatgttttagcaaaagctcctgagtttaaaaagtttgaagatattttaagtcttactacatttggaaaatacataaatcaggaaggaataactacatataataataacgtagttgcaacaattaatcttgaacttaatacgtaccatcagcataatccaaaaatcttttctcgcctgccaaagttaaaattgctttataaacaaattggttcaccaaaagaggacaaacgcatttttactattgaaaaaagaacggaatggcagagtttggaagacttaatacaaaaacagaataaagttgttgaacacgaaaaaaagaatgttgaaatcctgtcaaatttgaaagcagcatacatttcttttttcacgaacacagatgaaacaatcttaagcgaggtatatttcaataagcgttctcttaatacaatttcttctttctggtttacgggtggctggcaaacactgcttcttaaactaaaagagtttaaattggccaatcaaaacaaagatggtgatatagtagtccctaaagcattatcccttgctgaactaaaacaggtgcttgattcgttagaagagcaagaccctgctgttaatcatttatttaaggaaatgtactcagattgttacaaagaaaacctatggcagacctttatagctatctggcaatgtgaaattacatcaaaatttaacctgctcgaagggtatattcaagaatgtaatgctgttaaagaagacacctttgataaaaaaaagcataaaaatattatcaaaaacatctgcgatacatacctggatattgagcagatatcaaaatacataatagtacatgaaagtcttcctaaatatgatgcgctatatgatgcggtaatactttatttgcaggaatcttctttacgcagttattacgatgccttccgcaaccttattagcaagcgacctgttaacgaagaaaaagttaagctcaactttcagaactctaccctgcttgatggctgggatatgaataaagaaagcgctaacttatgcgtattactgaaaaacaatataggtgaatacttccttgctgtaatgaataaaaagagcaacatggtttttgatcagaagaaaaactctgccctttactctgctgggaatgaaagtagttttcagaagctggagtataaactgttgcctgggcctaacaaaatgctgccaaaagtaatttttgcaaaatcgaacgaaaaatatttcatcataccggaagaaattgtgcagattagagaagaagaatcgtttaaaaaaggaaaaaaatttgataagcatgcattgaaaacgtggatcaggtttatgcaggaatcaattgaaaaatacccaggttggaagacattcgactttacctttaaaaaaccggaagagtacgaagatgtcagcaagttctataaagatgtagaagaacaggggtataaactaaactggaaagatattaacgaggaagagctcctgtcacttgtagaacaaaaaaaagtatatctgtttcagataaaaagcaaagatatcggagaaacaaaggagcacggcaacaagaaccttcacacattgttatttttagaactcctcaaaccggaaaataccagcaggttaaagctactgggcggtggcgaaatgttttatcgtgcgccaagtatggaaaaggtatacaaaaccgtaaatgaaaaacaggttctggattcaaaaggtaaccccattttagaagcaaaacggtactatgaaccaaagtttttccttcacttccctattcaggtcaaagggagcgaaaatggttataaaacagaaatgaatccgaaaatattgcgggcaattagcacttcaaaagaagtaaatataataggaatagaccgtggagaaaagcatttactctattattacgttataaagccagacggaactccaattactcaaggaagcctgaatacaattagtttaggtttagataaaaatcaaaatcccagacttgttgacgagcgtaccttcaagattttggagagagattccaagggaaaaccatcaaaaatatcagattttgaatctacagggaaaaaagttgattacatagattatcacaatatacttacctattacgaaacaaaacgcaatatagcacgccgttcgtgggatactattggggcaataaaaaactttaaagaggggtacttgtctcaggcgattcaccagatttatcagcttatgttgaagtataacgctgtggtagttttggaagatcttaatacggagtttaaggcaaaacgaaccgcaaaagttgaaaaatccgtgtacgaaaagtttgaaattgcccttgctaaaaaactgaaccacttaattattaaaggaactgaccctgcagaagcaggaagcgtaataaatccgtatcagcttactccagcaattacagctgatacattaagcgactttaagaaatcaaaacaatggggtccgcttttctatattagagcaaactatacctctacgactgaccctataaccggctggcgtaaacacatatatatcccgtccggagcttcagataaagaaattaaaacatatttctgtaaacagggcgaaaaagaacctttgattcagatttcatatgatacagcgcttaccgcgtttgcatttacctatacccatgaaggcaaagaatggacattacacgcaacgaaagatactcagcgtatgcgttatgacagtaagaagcggaagatggaacccgtagaaatatttgatagactacgagagctttttatagattttagtttcgaagaatcgttaacagatcaactagaagcaacactttcctttgactggaaaacactggcctttttgtggacaatgttaaaccagatacgtaataccgacagagaagcagaagggaatgacggtgactttattcagtctccggttgctccgttttatgatagtcgagatccggaaaataaaacaaatggacttcctgttaacggagatgctaatggggctttcaatatagccagaaaaggtgcaatcctgataaaacgtattcaagaatatgcaaaaaaagaccccacctttgaaaagatgagagaaaaagatggtctcaatttgtatatatctgatgcagagtgggatacagaaataagctaa

SEQ ID NO: 65 - DNA, 3783 bp, synthetic construct
  source 1..3783: mol_type = other DNA
acaaacactagcatcttcaagacattcaccaaccaatacagcctctccaagaccctgcggtttgagctcagaccccaccctatgacctccggcctggacgacatcatcagcctggacaccggaatcaaaaagctgtacgagaacgaaatgaagcctctgttcgacgagctgcacttcgagttcatcagccagagcctggtccaggtcagcttccctagcgagaagctcgaagtgctgctgaacaagtaccggagcctgaaggaccagaaagctaagaacatcgagaaggaactggagggccccctgcaggagctgagaaccatcatcaccgacaccttcgagagcaccggcaacaactggaagaaagagtggctgcagcaggggttcaagatcaaaagcagtggatacaaggtgctgacagaggagggcatcctggaagtgctttccgtgcggaagaaggataaggccgatgctataaacaagttcaaaggattcttcacctacttcagcggcttcaacatgaacagagagaactactacagcagcgaagataaaaaaacagccgtggcctacagagtgatcaacgagaacctgatccggtacatggataacatcctgctcctgcagaacgtgctggccaaagcccctgagttcaagaaatttgaagatatcctgagtctgaccaccttcggcaagtacatcaaccaggagggcatcacaacctacaacaacaacgttgtggccaccatcaacctggagctgaacacctaccaccagcacaacccaaaaatcttcagcagactgcccaaactgaagctgctgtacaagcagatcggttctccaaaggaggacaagcgcatcttcaccatcgagaagagaacagaatggcagagcctggaggacctgatccagaagcagaacaaggtcgtggaacacgaaaagaagaacgtggagatcctgtctaatctgaaggccgcctatatcagcttcttcacaaacaccgacgaaaccatcctgtctgaggtgtacttcaacaagagaagcctgaatacgatcagcagcttctggttcaccggcggatggcaaaccctgctgctgaaactgaaggaatttaagctggctaatcagaacaaagacggcgatatcgtggttcccaaggccctgagcctggccgagctgaagcaggtgctggactccctggaagagcaggaccccgccgtgaatcacctgttcaaggaaatgtacagcgactgctacaaggaaaacctgtggcaaacatttatcgccatctggcaatgtgaaatcacaagcaagttcaacctgctggagggctatatccaagagtgcaacgccgtgaaagaggacacctttgacaagaaaaagcacaaaaacatcatcaagaacatctgcgacacgtacctggacattgagcagatcagtaagtacatcatcgtgcacgaaagcctgcctaaatacgacgccctctatgatgccgtcatcctgtacctgcaggagtctagtctgcggtcctactacgacgcctttagaaacctgatttctaagcggccagtgaacgaggaaaaggtgaagctgaatttccagaatagcaccctgctggatggctgggacatgaataaagaaagcgccaatctttgtgtgctgctgaagaacaacatcggagagtactttctggccgtgatgaacaaaaaaagcaacatggtttttgaccagaaaaaaaacagcgccctgtatagcgctggcaatgaatctagcttccagaagctggagtacaagctgttgcccggccctaacaagatgctgcctaaggtgatctttgccaagtccaatgagaagtacttcatcatccctgaggagatcgtgcagatcagggaggaagagagcttcaagaaaggcaaaaaattcgataagcacgcgctgaaaacctggatcagattcatgcaggagtctatcgagaagtatcctggctggaaaacctttgacttcacattcaaaaagcctgaggaatacgaggatgtgtccaagttctacaaagacgtggaagagcagggctacaaactgaactggaaggatatcaacgaggaagaactgctgagcctggtggaacagaagaaggtgtacctttttcagatcaagtccaaagacataggcgagacaaaggaacacggaaataagaacctgcacaccctgctcttcctagaattgctgaagcctgagaacacaagtcggctgaagctgttgggcggcggagaaatgttctaccgggccccttctatggaaaaagtctacaaaacagtgaacgagaagcaggtgctggattctaaaggcaaccctatcctggaggccaagcgctactacgagcctaagttttttctgcatttccccatccaggtgaagggctctgagaacggctataagaccgagatgaaccccaaaatcctcagagccatcagcaccagcaaggaagtgaacatcattggcatcgacagaggcgagaagcacctgctgtactattacgtgatcaagcccgacggaacacctatcacccagggcagcctgaacaccatctccctgggccttgataagaatcaaaatcctagactggtggacgagagaaccttcaagatcctggaaagagatagcaagggcaagccaagcaagatctcagattttgaaagcacaggcaagaaggtcgactacatcgactaccacaacatcctgacatactatgaaaccaagagaaatatcgccagaagaagctgggacacaattggcgccatcaagaatttcaaggagggatacctctctcaggccatccaccagatctaccagctgatgctgaaatataacgccgtggtggtgctagaggacctgaacaccgagttcaaggcaaagagaaccgccaaggtggaaaaaagcgtgtacgaaaagtttgagatagctctggccaagaagctgaatcacctgatcatcaagggcaccgacccagccgaggccggatctgtgatcaacccttaccagctgacccctgctattacagccgacacactgagcgatttcaagaagagcaaacaatggggccctctgttctacatccgggccaactacaccagcacaaccgaccctatcacaggctggagaaagcacatctacatccccagcggagccagtgacaaggaaatcaagacctacttctgcaagcagggcgagaaggagcctctgatccagattagctacgacaccgccctgaccgccttcgccttcacatacacccacgaaggcaaggagtggaccctacatgccacaaaggatacccaaagaatgcggtacgacagcaagaagagaaagatggaacccgtggaaatcttcgacagactgagagagctgttcatcgacttctctttcgaggaaagcctgaccgaccagctggaggcaaccctgtccttcgactggaaaaccctggcttttctgtggacaatgctgaatcagatcagaaacaccgatagagaggctgaaggcaacgacggcgacttcatccagtctcctgtggcccctttctatgatagccgggacccagagaacaagaccaatggcctgcccgttaacggcgacgccaacggcgccttcaacatcgctagaaagggggctatcctgatcaagagaatccaggaatacgccaagaaggaccctacattcgagaagatgcgggaaaaggacggtttaaacctgtacatcagcgatgctgagtgggataccgagatcagc

SEQ ID NO: 66 - DNA, 3906 bp, synthetic construct
  source 1..3906: mol_type = other DNA
atgggctccggaacaaacactagcatcttcaagacattcaccaaccaatacagcctctccaagaccctgcggtttgagctcagaccccaccctatgacctccggcctggacgacatcatcagcctggacaccggaatcaaaaagctgtacgagaacgaaatgaagcctctgttcgacgagctgcacttcgagttcatcagccagagcctggtccaggtcagcttccctagcgagaagctcgaagtgctgctgaacaagtaccggagcctgaaggaccagaaagctaagaacatcgagaaggaactggagggccccctgcaggagctgagaaccatcatcaccgacaccttcgagagcaccggcaacaactggaagaaagagtggctgcagcaggggttcaagatcaaaagcagtggatacaaggtgctgacagaggagggcatcctggaagtgctttccgtgcggaagaaggataaggccgatgctataaacaagttcaaaggattcttcacctacttcagcggcttcaacatgaacagagagaactactacagcagcgaagataaaaaaacagccgtggcctacagagtgatcaacgagaacctgatccggtacatggataacatcctgctcctgcagaacgtgctggccaaagcccctgagttcaagaaatttgaagatatcctgagtctgaccaccttcggcaagtacatcaaccaggagggcatcacaacctacaacaacaacgttgtggccaccatcaacctggagctgaacacctaccaccagcacaacccaaaaatcttcagcagactgcccaaactgaagctgctgtacaagcagatcggttctccaaaggaggacaagcgcatcttcaccatcgagaagagaacagaatggcagagcctggaggacctgatccagaagcagaacaaggtcgtggaacacgaaaagaagaacgtggagatcctgtctaatctgaaggccgcctatatcagcttcttcacaaacaccgacgaaaccatcctgtctgaggtgtacttcaacaagagaagcctgaatacgatcagcagcttctggttcaccggcggatggcaaaccctgctgctgaaactgaaggaatttaagctggctaatcagaacaaagacggcgatatcgtggttcccaaggccctgagcctggccgagctgaagcaggtgctggactccctggaagagcaggaccccgccgtgaatcacctgttcaaggaaatgtacagcgactgctacaaggaaaacctgtggcaaacatttatcgccatctggcaatgtgaaatcacaagcaagttcaacctgctggagggctatatccaagagtgcaacgccgtgaaagaggacacctttgacaagaaaaagcacaaaaacatcatcaagaacatctgcgacacgtacctggacattgagcagatcagtaagtacatcatcgtgcacgaaagcctgcctaaatacgacgccctctatgatgccgtcatcctgtacctgcaggagtctagtctgcggtcctactacgacgcctttagaaacctgatttctaagcggccagtgaacgaggaaaaggtgaagctgaatttccagaatagcaccctgctggatggctgggacatgaataaagaaagcgccaatctttgtgtgctgctgaagaacaacatcggagagtactttctggccgtgatgaacaaaaaaagcaacatggtttttgaccagaaaaaaaacagcgccctgtatagcgctggcaatgaatctagcttccagaagctggagtacaagctgttgcccggccctaacaagatgctgcctaaggtgatctttgccaagtccaatgagaagtacttcatcatccctgaggagatcgtgcagatcagggaggaagagagcttcaagaaaggcaaaaaattcgataagcacgcgctgaaaacctggatcagattcatgcaggagtctatcgagaagtatcctggctggaaaacctttgacttcacattcaaaaagcctgaggaatacgaggatgtgtccaagttctacaaagacgtggaagagcagggctacaaactgaactggaaggatatcaacgaggaagaactgctgagcctggtggaacagaagaaggtgtacctttttcagatcaagtccaaagacataggcgagacaaaggaacacggaaataagaacctgcacaccctgctcttcctagaattgctgaagcctgagaacacaagtcggctgaagctgttgggcggcggagaaatgttctaccgggccccttctatggaaaaagtctacaaaacagtgaacgagaagcaggtgctggattctaaaggcaaccctatcctggaggccaagcgctactacgagcctaagttttttctgcatttccccatccaggtgaagggctctgagaacggctataagaccgagatgaaccccaaaatcctcagagccatcagcaccagcaaggaagtgaacatcattggcatcgacagaggcgagaagcacctgctgtactattacgtgatcaagcccgacggaacacctatcacccagggcagcctgaacaccatctccctgggccttgataagaatcaaaatcctagactggtggacgagagaaccttcaagatcctggaaagagatagcaagggcaagccaagcaagatctcagattttgaaagcacaggcaagaaggtcgactacatcgactaccacaacatcctgacatactatgaaaccaagagaaatatcgccagaagaagctgggacacaattggcgccatcaagaatttcaaggagggatacctctctcaggccatccaccagatctaccagctgatgctgaaatataacgccgtggtggtgctagaggacctgaacaccgagttcaaggcaaagagaaccgccaaggtggaaaaaagcgtgtacgaaaagtttgagatagctctggccaagaagctgaatcacctgatcatcaagggcaccgacccagccgaggccggatctgtgatcaacccttaccagctgacccctgctattacagccgacacactgagcgatttcaagaagagcaaacaatggggccctctgttctacatccgggccaactacaccagcacaaccgaccctatcacaggctggagaaagcacatctacatccccagcggagccagtgacaaggaaatcaagacctacttctgcaagcagggcgagaaggagcctctgatccagattagctacgacaccgccctgaccgccttcgccttcacatacacccacgaaggcaaggagtggaccctacatgccacaaaggatacccaaagaatgcggtacgacagcaagaagagaaagatggaacccgtggaaatcttcgacagactgagagagctgttcatcgacttctctttcgaggaaagcctgaccgaccagctggaggcaaccctgtccttcgactggaaaaccctggcttttctgtggacaatgctgaatcagatcagaaacaccgatagagaggctgaaggcaacgacggcgacttcatccagtctcctgtggcccctttctatgatagccgggacccagagaacaagaccaatggcctgcccgttaacggcgacgccaacggcgccttcaacatcgctagaaagggggctatcctgatcaagagaatccaggaatacgccaagaaggaccctacattcgagaagatgcgggaaaaggacggtttaaacctgtacatcagcgatgctgagtgggataccgagatcagctctagaaagcggacagcagacggctccgaatttgaaagccctaagaaaaagagaaaggtgggatccggcaaacctatccccaatcccctgctgggcctggacagcacctga

SEQ ID NO: 67 - protein, 1132 aa, unidentified
AYQVVKCLINDYCQNEIIAPQLQKVSCDNTWIVKLREFQEAANWEAQKIIQQDLIGIINKKLPKKFNSKALIEAIPDYLQGKSKEDLQRMLSGIHDYEIKVKNQNLQVAWNNGLEDFCNLCYQQFRGFSGYLDALSENLKFLFSGRKNGIAYRIVYQNLVTFERNRRAYESLILINETFRVQDEALLLNYSSSLTQEGINTYNERIGQLVKNLKEFGDTDRSFRNWHRRFKKLNKQILSPRVAPPWLARAYRSDEEMVMSLQSFLDEFNPLKPRLKQLIANLESYDEHIYYFRKSLSLLSVTLRNDYKALDEELSIPQEQANCRSLSLSWIPFRQELINEIERIIDSSYTDIEKCLASASEYLNTERAKRNDYRLDNTVSFTIKKLMDVFLSLYRAVKPLTGTGEEEDRNEDFYDEFTTIWDVLQYVQKLYNAVFAWLNKKPYENNSYPAYLDEFTLLKNWKEKAAYIKRNGKFYFIMFNGIDEQDIIEHRGDSAILYHVESQSPDRIKANLTKQFVFSKKANAGKGRPNPSKAKFVRDNPEFQADWERVKTEAYKVAGNTEALAHAIRYFQRCLQSHPDYNRFPFNFRPANDYTSLDDFVDSIKDKLFMMEETAINWSYVRQLAEEGTIYLFKLYNKDYAKNRVGGSKPNLHTLYWEAMFSSENLRENNIKLEEPKLFYREVATNRDGELNMRLIPHRYATDQLELHVPIHLNVNATASSDINMMVLDAIREGSIENVIGIDRGERNLLYYSVLRLSDGEIVDQKSLNITFNDVDYHAKLSTKEEEIHDEQREWKAKTSIRKLKEGYLSQAIHQLTSLIVKYHAVVVLEDLSEDFYSKRQKINKQIYQIFEKRLIEKLSYFVDKDAAEGQAGNIYSALQLSSPNLVRKDNKKIFQNGIVFFVPPEYTSAIDPVTGFCNLFDKNRVRNICELLYRFENICYNRKNDRFEFTWDYRNVMTYTRLEQDNISHLWTACSLGNRIEWSGSERNKNRRCEIVNLTQSMKVLFEKHGIQYQTGKDVREAVCSIRNNDFKKELKRLFFLMLSLRNSIVDGKVKKDYILSPVQNQRGSFFDSREYEELDNPKLPKCGDANGAYNIARKGILTIRKLENGNEKALTLDEWVISTQKGNIRM

SEQ ID NO: 68 - protein, 1133 aa, unidentified
MAYQVVKCLINDYCQNEIIAPQLQKVSCDNTWIVKLREFQEAANWEAQKIIQQDLIGIINKKLPKKFNSKALIEAIPDYLQGKSKEDLQRMLSGIHDYEIKVKNQNLQVAWNNGLEDFCNLCYQQFRGFSGYLDALSENLKFLFSGRKNGIAYRIVYQNLVTFERNRRAYESLILINETFRVQDEALLLNYSSSLTQEGINTYNERIGQLVKNLKEFGDTDRSFRNWHRRFKKLNKQILSPRVAPPWLARAYRSDEEMVMSLQSFLDEFNPLKPRLKQLIANLESYDEHIYYFRKSLSLLSVTLRNDYKALDEELSIPQEQANCRSLSLSWIPFRQELINEIERIIDSSYTDIEKCLASASEYLNTERAKRNDYRLDNTVSFTIKKLMDVFLSLYRAVKPLTGTGEEEDRNEDFYDEFTTIWDVLQYVQKLYNAVFAWLNKKPYENNSYPAYLDEFTLLKNWKEKAAYIKRNGKFYFIMFNGIDEQDIIEHRGDSAILYHVESQSPDRIKANLTKQFVFSKKANAGKGRPNPSKAKFVRDNPEFQADWERVKTEAYKVAGNTEALAHAIRYFQRCLQSHPDYNRFPFNFRPANDYTSLDDFVDSIKDKLFMMEETAINWSYVRQLAEEGTIYLFKLYNKDYAKNRVGGSKPNLHTLYWEAMFSSENLRENNIKLEEPKLFYREVATNRDGELNMRLIPHRYATDQLELHVPIHLNVNATASSDINMMVLDAIREGSIENVIGIDRGERNLLYYSVLRLSDGEIVDQKSLNITFNDVDYHAKLSTKEEEIHDEQREWKAKTSIRKLKEGYLSQAIHQLTSLIVKYHAVVVLEDLSEDFYSKRQKINKQIYQIFEKRLIEKLSYFVDKDAAEGQAGNIYSALQLSSPNLVRKDNKKIFQNGIVFFVPPEYTSAIDPVTGFCNLFDKNRVRNICELLYRFENICYNRKNDRFEFTWDYRNVMTYTRLEQDNISHLWTACSLGNRIEWSGSERNKNRRCEIVNLTQSMKVLFEKHGIQYQTGKDVREAVCSIRNNDFKKELKRLFFLMLSLRNSIVDGKVKKDYILSPVQNQRGSFFDSREYEELDNPKLPKCGDANGAYNIARKGILTIRKLENGNEKALTLDEWVISTQKGNIRM

SEQ ID NO: 69 - protein, 1172 aa, synthetic construct
MGSGAYQVVKCLINDYCQNEIIAPQLQKVSCDNTWIVKLREFQEAANWEAQKIIQQDLIGIINKKLPKKFNSKALIEAIPDYLQGKSKEDLQRMLSGIHDYEIKVKNQNLQVAWNNGLEDFCNLCYQQFRGFSGYLDALSENLKFLFSGRKNGIAYRIVYQNLVTFERNRRAYESLILINETFRVQDEALLLNYSSSLTQEGINTYNERIGQLVKNLKEFGDTDRSFRNWHRRFKKLNKQILSPRVAPPWLARAYRSDEEMVMSLQSFLDEFNPLKPRLKQLIANLESYDEHIYYFRKSLSLLSVTLRNDYKALDEELSIPQEQANCRSLSLSWIPFRQELINEIERIIDSSYTDIEKCLASASEYLNTERAKRNDYRLDNTVSFTIKKLMDVFLSLYRAVKPLTGTGEEEDRNEDFYDEFTTIWDVLQYVQKLYNAVFAWLNKKPYENNSYPAYLDEFTLLKNWKEKAAYIKRNGKFYFIMFNGIDEQDIIEHRGDSAILYHVESQSPDRIKANLTKQFVFSKKANAGKGRPNPSKAKFVRDNPEFQADWERVKTEAYKVAGNTEALAHAIRYFQRCLQSHPDYNRFPFNFRPANDYTSLDDFVDSIKDKLFMMEETAINWSYVRQLAEEGTIYLFKLYNKDYAKNRVGGSKPNLHTLYWEAMFSSENLRENNIKLEEPKLFYREVATNRDGELNMRLIPHRYATDQLELHVPIHLNVNATASSDINMMVLDAIREGSIENVIGIDRGERNLLYYSVLRLSDGEIVDQKSLNITFNDVDYHAKLSTKEEEIHDEQREWKAKTSIRKLKEGYLSQAIHQLTSLIVKYHAVVVLEDLSEDFYSKRQKINKQIYQIFEKRLIEKLSYFVDKDAAEGQAGNIYSALQLSSPNLVRKDNKKIFQNGIVFFVPPEYTSAIDPVTGFCNLFDKNRVRNICELLYRFENICYNRKNDRFEFTWDYRNVMTYTRLEQDNISHLWTACSLGNRIEWSGSERNKNRRCEIVNLTQSMKVLFEKHGIQYQTGKDVREAVCSIRNNDFKKELKRLFFLMLSLRNSIVDGKVKKDYILSPVQNQRGSFFDSREYEELDNPKLPKCGDANGAYNIARKGILTIRKLENGNEKALTLDEWVISTQKGNIRMSRKRTADGSEFESPKKKRKVGSGKPIPNPLLGLDST

SEQ ID NO: 70 - DNA, 3402 bp, unidentified
  source 1..3402: mol_type = unassigned DNA
atggcataccaagtggttaaatgcctaatcaacgactattgccagaatgaaatcattgcacctcaattgcagaaagtttcctgtgataacacttggattgtaaaacttcgcgagtttcaagaggctgccaattgggaagcccaaaaaattatccagcaagatcttattggtatcataaacaagaaacttcctaaaaagttcaatagcaaggcattgatagaagccattcctgactatttacaaggcaagtctaaagaagatctgcaacgtatgttgagtggtatacatgactatgagattaaggtaaaaaatcagaaccttcaggtggcttggaataatgggttagaagatttttgtaacctctgctatcaacaatttagaggattttctggctatcttgacgctttatctgagaacctgaaatttctattctcgggcagaaaaaatggtatagcctatagaatagtgtatcagaaccttgttacatttgagaggaataggagagcttatgaatccctaatattaataaatgagacttttagggtacaagatgaggctctacttcttaattactccagtagtctgacccaagaaggtatcaacacctataatgaacgaatagggcaacttgtcaaaaatctgaaagaatttggcgatacagacagatctttcagaaactggcatcgccgattcaagaaactgaacaagcaaatcctaagccctcgtgttgctccaccttggttggcacgcgcctacagaagcgatgaagagatggtgatgtcgctacagtcttttctcgacgagttcaatccattaaaacctcgtttgaagcaacttattgctaatctggaatcttacgatgagcatatctattacttccgcaagtctctttctctattatcggtgaccttgaggaatgattataaggcacttgatgaagaactctcaataccacaagaacaggccaattgcagaagtttaagcctttcgtggattccgtttcgccaagaattgataaacgaaatagaacgaattattgacagttcatatacagacatagagaagtgtcttgcctctgcctcggaatatctgaacacggagagagcaaaacggaacgactatcgtctagataatactgtgtctttcacaatcaagaaactgatggacgtattcctgtcattgtatcgtgcggtgaagcctctgactggaacaggagaggaggaggatcgaaacgaggacttctatgatgagtttacaacaatctgggatgtgcttcaatatgtacaaaaactttataatgcagtttttgcatggctgaacaagaagccttatgagaacaacagctatcctgcctatttggacgagtttacacttcttaaaaactggaaggagaaagccgcgtatataaaacggaatgggaagttctattttatcatgttcaatggtattgatgaacaagacattatcgagcatcgaggtgattctgcaatcttgtatcatgtggaaagtcaatcccccgataggattaaggcaaatctcaccaaacaatttgttttttccaaaaaagcaaatgcaggaaaggggcgaccaaatccttctaaagccaaattcgtgcgtgacaatccagaattccaagctgactgggaacgtgtgaaaactgaagcatataaagtagctggaaacacagaagcgcttgctcatgccattcgatattttcaacgctgccttcaatcacatcctgactataataggtttccgttcaattttagaccagcgaatgactacactagtttagatgattttgttgactccattaaagacaaattgtttatgatggaagaaactgctattaactggtcgtatgtgaggcaattagcagaagaaggaacaatttacttgtttaaactctacaataaagattatgccaagaatagagttggcgggtctaaacccaacttgcatacgctctattgggaggcgatgttcagctctgagaaccttcgtgaaaataatataaagttggaggaacccaaactcttctatcgtgaagttgcaactaaccgtgatggtgaattgaatatgcgcttgatacctcacagatatgcaacagaccaacttgagctgcatgttccaattcacttaaatgtgaatgcaaccgcttcaagcgatataaatatgatggtgttggatgcaatacgagaagggagtattgaaaatgtcattggtattgaccgtggagagaggaaccttctctactattcagtcttgcggttgtcagatggtgaaattgttgaccaaaaaagtttgaatattactttcaatgatgttgactaccacgccaaactgtcgactaaagaggaggaaatccatgacgaacaaagagaatggaaagcaaaaacaagtattcggaaactgaaagaaggataccttagtcaagctatccaccaactaacatcgctgattgtcaagtaccatgctgtggtagtgctagaagacttatcagaggacttctattcgaagcgccagaagataaacaagcaaatctatcagatatttgaaaaaaggctgatagaaaaactgagttattttgtcgataaggatgctgcagaaggtcaggcaggcaatatatattcagcattgcagttgtcaagccccaacttggtgaggaaagataataaaaaaatctttcagaacggcatcgtcttttttgtgccacctgaatatacaagtgccattgaccctgtaacagggttctgcaatctctttgacaagaatcgggtaagaaatatttgcgaacttctctacagatttgaaaacatctgctataataggaaaaatgaccgatttgagttcacatgggactatcgtaatgttatgacttatacgcgtctggagcaggacaatatttcacatctttggacagcatgctctttaggaaacaggattgaatggtctggtagcgaacgtaataaaaacagaaggtgcgaaattgtaaaccttacgcaatctatgaaagttttgtttgaaaaacatggtatccaataccaaacaggaaaagatgtaagggaggctgtatgcagcataagaaacaacgattttaaaaaagaattgaagcgcctgttcttcttgatgttatctttaaggaatagcattgttgatggaaaagtgaaaaaagactatatattatcccccgttcagaaccaacgaggcagttttttcgatagtagagaatatgaagagttggacaatccaaaactccctaaatgtggagatgcaaatggcgcatataatattgcaaggaaagggatactgacaattagaaagttggaaaatggcaatgaaaaggcattaacccttgatgagtgggttatttctacgcaaaaagggaatatacgcatgtaa

SEQ ID NO: 71 - DNA, 3396 bp, synthetic construct
  source 1..3396: mol_type = other DNA
gcctaccaggtggtgaaatgcctgattaacgactactgccagaacgagatcatcgcccctcagctgcaaaaggtgagctgcgacaatacctggatcgtgaagctcagagagttccaggaggccgcaaactgggaagcccagaagatcatccagcaggacctgatcggcattatcaataagaaactgcctaagaaattcaactctaaggccctgatcgaggctatacctgattacctccagggcaagagcaaggaagatctgcagagaatgctgtccggcatccacgactatgagatcaaggtgaagaaccagaacctgcaggtagcttggaacaatggcctggaagatttctgtaacttgtgctaccaacaatttagaggcttttccggctaccttgatgctctgtcagaaaatctgaagttcctgttcagcggcagaaaaaacggcatcgcctacaggatcgtctaccagaacctggtgaccttcgagcggaaccggagagcttacgagagcctgatcctgatcaacgagacatttagagtgcaggacgaggccctgctgctcaactactctagctctctgacacaggagggaatcaacacgtacaacgagcggatcggccagctggtgaagaacctgaaggagttcggcgacaccgaccggagctttcggaactggcacagacggttcaagaaactgaacaagcagatcctgagccctagagtggcccctccttggctggctcgtgcctacagaagcgatgaggaaatggtgatgagcctgcagagcttcctggatgagttcaaccctctgaaacctagactcaaacagctgatcgccaatctggagtcctacgacgagcacatctactacttcagaaagtccctgtctctgctgtcagtgacactgaggaacgactataaggcactggatgaagagctgagcatccctcaggagcaggccaactgcagatctcttagcctgagctggattcctttcagacaggaactgatcaacgagatcgagagaatcatcgatagcagctacacagacattgagaagtgcctggccagcgcctccgagtacctgaacaccgagagagccaagagaaacgactaccggctagataataccgtgtccttcaccatcaagaagctgatggacgtgttcctgagcctgtaccgcgccgtgaagcctctgaccggaacaggcgaagaggaggacagaaatgaagatttctacgacgagttcaccaccatctgggatgtgctgcaatacgtgcagaagctgtacaacgctgttttcgcctggctgaacaagaagccctacgagaacaatagctaccctgcctacctggatgaatttaccctgctgaagaactggaaggaaaaggccgcctacatcaagaggaatggaaaattctacttcatcatgttcaacggcatcgacgagcaggatatcatcgaacacagaggagattctgccatcctgtaccatgtggaaagccagagccctgatagaatcaaggccaatctgaccaagcagttcgtgttcagcaagaaagccaatgccggcaagggccggcccaatcccagcaaggccaagttcgtgagagataaccccgagtttcaggccgactgggagcgggtgaaaaccgaggcctacaaggtggccggaaacaccgaggccctggcccacgccatcagatacttccaaagatgcctgcaaagccaccccgattataatcggttccccttcaacttcagacctgccaacgactacacatctctggatgacttcgtggacagcatcaaggacaagctgttcatgatggaagaaaccgccatcaactggagttatgtgagacagctggccgaagaaggcacaatctacctgttcaagctgtataacaaagactacgccaagaaccgggtgggcggcagcaagcctaacctgcacaccctgtactgggaggccatgttcagctctgagaatctgagagaaaacaacatcaaactggaagaacccaaactgttctacagagaggtggccacaaaccgggacggcgagctgaacatgagactgatcccccacagatacgccaccgaccagctggaactgcacgtgcctatccacctgaatgtgaacgccacagccagcagcgacatcaacatgatggtccttgatgccatccgggaaggatctattgagaacgtgatcggcatcgaccggggagaacggaacctgctgtactacagcgtcctgcgactgtccgacggcgagatcgtggaccagaagagcctgaatatcacctttaacgatgtggactaccacgcaaagttgtctaccaaggaggaagaaatccatgatgagcagagagagtggaaagccaagacctccatcagaaagctgaaggaaggttacctgtctcaggctatccaccagctgaccagcctgatcgtgaagtaccacgctgtggtagtgctggaagatctgagcgaagatttctacagcaagcggcagaaaatcaacaagcagatctaccagattttcgagaaaagacttatcgagaagctgagctactttgtggacaaagacgccgccgagggccaggcaggcaacatctacagcgccctgcagctgagctccccaaatctggtgagaaaggacaacaagaagatcttccagaacggcatcgtgttcttcgtgccacctgagtacacgagtgcgattgaccccgtgaccggcttctgcaacctgtttgacaagaacagagtgcgcaatatctgtgagctgctctacagattcgaaaacatttgctacaacagaaagaatgaccggtttgagttcacatgggactatagaaacgtgatgacctacaccagacttgagcaggacaacatctctcacctgtggaccgcttgtagcctcggcaaccggatcgagtggagcggctctgaaagaaataagaacagaagatgcgagatcgtgaacctgacacaaagcatgaaggtcctgtttgagaagcacggcatccagtaccagaccggcaaggacgtgcgggaagctgtgtgtagtatcagaaacaacgactttaagaaagaactgaagagactgtttttcctgatgctgagcctgcgtaacagcatcgtggatggaaaggtgaaaaaggactacatcctgagcccagtgcaaaaccagcggggtagctttttcgactccagagaatatgaagaactggacaacccgaagttgcctaagtgcggggacgccaacggcgcctacaacatcgccagaaaaggaatcctgacaatcagaaagctggagaacggcaacgagaaagccctgaccctggacgaatgggtgatcagcacccagaagggcaacatcagaatg

SEQ ID NO: 72 - DNA, 3519 bp, synthetic construct
  source 1..3519: mol_type = other DNA
atgggctccggagcctaccaggtggtgaaatgcctgattaacgactactgccagaacgagatcatcgcccctcagctgcaaaaggtgagctgcgacaatacctggatcgtgaagctcagagagttccaggaggccgcaaactgggaagcccagaagatcatccagcaggacctgatcggcattatcaataagaaactgcctaagaaattcaactctaaggccctgatcgaggctatacctgattacctccagggcaagagcaaggaagatctgcagagaatgctgtccggcatccacgactatgagatcaaggtgaagaaccagaacctgcaggtagcttggaacaatggcctggaagatttctgtaacttgtgctaccaacaatttagaggcttttccggctaccttgatgctctgtcagaaaatctgaagttcctgttcagcggcagaaaaaacggcatcgcctacaggatcgtctaccagaacctggtgaccttcgagcggaaccggagagcttacgagagcctgatcctgatcaacgagacatttagagtgcaggacgaggccctgctgctcaactactctagctctctgacacaggagggaatcaacacgtacaacgagcggatcggccagctggtgaagaacctgaaggagttcggcgacaccgaccggagctttcggaactggcacagacggttcaagaaactgaacaagcagatcctgagccctagagtggcccctccttggctggctcgtgcctacagaagcgatgaggaaatggtgatgagcctgcagagcttcctggatgagttcaaccctctgaaacctagactcaaacagctgatcgccaatctggagtcctacgacgagcacatctactacttcagaaagtccctgtctctgctgtcagtgacactgaggaacgactataaggcactggatgaagagctgagcatccctcaggagcaggccaactgcagatctcttagcctgagctggattcctttcagacaggaactgatcaacgagatcgagagaatcatcgatagcagctacacagacattgagaagtgcctggccagcgcctccgagtacctgaacaccgagagagccaagagaaacgactaccggctagataataccgtgtccttcaccatcaagaagctgatggacgtgttcctgagcctgtaccgcgccgtgaagcctctgaccggaacaggcgaagaggaggacagaaatgaagatttctacgacgagttcaccaccatctgggatgtgctgcaatacgtgcagaagctgtacaacgctgttttcgcctggctgaacaagaagccctacgagaacaatagctaccctgcctacctggatgaatttaccctgctgaagaactggaaggaaaaggccgcctacatcaagaggaatggaaaattctacttcatcatgttcaacggcatcgacgagcaggatatcatcgaacacagaggagattctgccatcctgtaccatgtggaaagccagagccctgatagaatcaaggccaatctgaccaagcagttcgtgttcagcaagaaagccaatgccggcaagggccggcccaatcccagcaaggccaagttcgtgagagataaccccgagtttcaggccgactgggagcgggtgaaaaccgaggcctacaaggtggccggaaacaccgaggccctggcccacgccatcagatacttccaaagatgcctgcaaagccaccccgattataatcggttccccttcaacttcagacctgccaacgactacacatctctggatgacttcgtggacagcatcaaggacaagctgttcatgatggaagaaaccgccatcaactggagttatgtgagacagctggccgaagaaggcacaatctacctgttcaagctgtataacaaagactacgccaagaaccgggtgggcggcagcaagcctaacctgcacaccctgtactgggaggccatgttcagctctgagaatctgagagaaaacaacatcaaactggaagaacccaaactgttctacagagaggtggccacaaaccgggacggcgagctgaacatgagactgatcccccacagatacgccaccgaccagctggaactgcacgtgcctatccacctgaatgtgaacgccacagccagcagcgacatcaacatgatggtccttgatgccatccgggaaggatctattgagaacgtgatcggcatcgaccggggagaacggaacctgctgtactacagcgtcctgcgactgtccgacggcgagatcgtggaccagaagagcctgaatatcacctttaacgatgtggactaccacgcaaagttgtctaccaaggaggaagaaatccatgatgagcagagagagtggaaagccaagacctccatcagaaagctgaaggaaggttacctgtctcaggctatccaccagctgaccagcctgatcgtgaagtaccacgctgtggtagtgctggaagatctgagcgaagatttctacagcaagcggcagaaaatcaacaagcagatctaccagattttcgagaaaagacttatcgagaagctgagctactttgtggacaaagacgccgccgagggccaggcaggcaacatctacagcgccctgcagctgagctccccaaatctggtgagaaaggacaacaagaagatcttccagaacggcatcgtgttcttcgtgccacctgagtacacgagtgcgattgaccccgtgaccggcttctgcaacctgtttgacaagaacagagtgcgcaatatctgtgagctgctctacagattcgaaaacatttgctacaacagaaagaatgaccggtttgagttcacatgggactatagaaacgtgatgacctacaccagacttgagcaggacaacatctctcacctgtggaccgcttgtagcctcggcaaccggatcgagtggagcggctctgaaagaaataagaacagaagatgcgagatcgtgaacctgacacaaagcatgaaggtcctgtttgagaagcacggcatccagtaccagaccggcaaggacgtgcgggaagctgtgtgtagtatcagaaacaacgactttaagaaagaactgaagagactgtttttcctgatgctgagcctgcgtaacagcatcgtggatggaaaggtgaaaaaggactacatcctgagcccagtgcaaaaccagcggggtagctttttcgactccagagaatatgaagaactggacaacccgaagttgcctaagtgcggggacgccaacggcgcctacaacatcgccagaaaaggaatcctgacaatcagaaagctggagaacggcaacgagaaagccctgaccctggacgaatgggtgatcagcacccagaagggcaacatcagaatgtctagaaagcggacagcagacggctccgaatttgaaagccctaagaaaaagagaaaggtgggatccggcaaacctatccccaatcccctgctgggcctggacagcacctga

SEQ ID NO: 73 - protein, 1283 aa, unidentified
ERMYEEFRNCYSVRKTLSFKAIPTEETKKHLQLQWEVLGDEIRFENYDKMKMVLDQLHQSYISRKLDNIGEENQKKIVEILEKLVLVMKKIDTTHQKDKEKAQNQLQSLQASLRKEIGMFFPKNEWQQLQGKNVFKKDGVLSEYNISEENKKNIQCYDGFMTFFKKYNETRANIYSTEEKSTAITFRIVNDNLPKYVRNADNYEQIKKLIPEALEEVEKTYPNLTNYFSIKNYLKYWSQKGIETYNTVIGEINKQVNLVVQQRKDSKFRKYKMQVLYQQILSDREEQSFVYQQDQEVFAAVNELAELVNGSAFNEAIELLKSPNINENEIFIPYAKLAEVSIKMKMGWNGLEEAFINDLQQQYPKKDHEKLVQKLKKEKKVFSLNEIKDVVMKIEHEEDWKFVSLLDCVEDYQKQLTETRDAYVEYAKTYAGSTGTSLQGNDVAPIKAFLDSCLQLVRWCKLFEYSDLYGNRDKIFYGGAESIILALDSLISVYNKTRNYVTMRPGQARKMHLMFNYPEFGDGFSNSKVDSYGTILLREGKKYYLAVIKKGIKVLLEDTINENDSYERLSYMLFPDVKKMIPKCSISTKKVKEHFENSDDDYTIRKGESYAKELLVKKEDYDLYFVNLYDDKKMFQKDYLSKTGDKKGYRQALERWIRFCIRFLQAYKSTKDYDLSELEPISNFRSLDEFYDKLDTLLYKIEWKTISREQIKQMESSGQLFLFELYNKDFSEHAKGKKNLFTLYWEQIFCEENLKQPVIKLCGGAEMFYRKVAIQKKYVHKKDSILVDKTYVDQNGVRKTLPDTIYKEWSDFMNKKITSVSQEASKYKGLVNCHEAKYDITKDKRYTEDQFEFHVPITLNYSALGKGQLNDSVLDCLCQKEKYNVIGIDRGERNLLAYCVVNQDGQILEQGTFNKIVGGNKQEVDYKQKLQEKEVNRQQARKEWKNIGKIKELKNGYLSQVIYQLTQMMVKYDAIVVMEDLNVGFKRGRFKVERQVYQKFEKALIDKLNYLVTKKDENQYGIEGSVSNAYQLTEKIKSFKDIGKQNGMIFYVPAGYTSKIDPTTGFVDVLNRTGLTNAKARKAFFENFDDINYSKEDNMFAFSFDYSKFKTFQEMHRKKWTVYTNGKKYIYSKKERKEKQIDVTELMKEELRKVGITEYDNLYSQITNVEDDKEHADFWKSLQFVFDRTMQLRSSQIDNGEDNLEDKIISPVKNAEGVFYESNGNYGDTSQPADADTNGAFHIARKGLLLAENVKKTGRGANGKWNSSVKNISNKDWFAFVQK

SEQ ID NO: 74 - protein, 1284 aa, unidentified
MERMYEEFRNCYSVRKTLSFKAIPTEETKKHLQLQWEVLGDEIRFENYDKMKMVLDQLHQSYISRKLDNIGEENQKKIVEILEKLVLVMKKIDTTHQKDKEKAQNQLQSLQASLRKEIGMFFPKNEWQQLQGKNVFKKDGVLSEYNISEENKKNIQCYDGFMTFFKKYNETRANIYSTEEKSTAITFRIVNDNLPKYVRNADNYEQIKKLIPEALEEVEKTYPNLTNYFSIKNYLKYWSQKGIETYNTVIGEINKQVNLVVQQRKDSKFRKYKMQVLYQQILSDREEQSFVYQQDQEVFAAVNELAELVNGSAFNEAIELLKSPNINENEIFIPYAKLAEVSIKMKMGWNGLEEAFINDLQQQYPKKDHEKLVQKLKKEKKVFSLNEIKDVVMKIEHEEDWKFVSLLDCVEDYQKQLTETRDAYVEYAKTYAGSTGTSLQGNDVAPIKAFLDSCLQLVRWCKLFEYSDLYGNRDKIFYGGAESIILALDSLISVYNKTRNYVTMRPGQARKMHLMFNYPEFGDGFSNSKVDSYGTILLREGKKYYLAVIKKGIKVLLEDTINENDSYERLSYMLFPDVKKMIPKCSISTKKVKEHFENSDDDYTIRKGESYAKELLVKKEDYDLYFVNLYDDKKMFQKDYLSKTGDKKGYRQALERWIRFCIRFLQAYKSTKDYDLSELEPISNFRSLDEFYDKLDTLLYKIEWKTISREQIKQMESSGQLFLFELYNKDFSEHAKGKKNLFTLYWEQIFCEENLKQPVIKLCGGAEMFYRKVAIQKKYVHKKDSILVDKTYVDQNGVRKTLPDTIYKEWSDFMNKKITSVSQEASKYKGLVNCHEAKYDITKDKRYTEDQFEFHVPITLNYSALGKGQLNDSVLDCLCQKEKYNVIGIDRGERNLLAYCVVNQDGQILEQGTFNKIVGGNKQEVDYKQKLQEKEVNRQQARKEWKNIGKIKELKNGYLSQVIYQLTQMMVKYDAIVVMEDLNVGFKRGRFKVERQVYQKFEKALIDKLNYLVTKKDENQYGIEGSVSNAYQLTEKIKSFKDIGKQNGMIFYVPAGYTSKIDPTTGFVDVLNRTGLTNAKARKAFFENFDDINYSKEDNMFAFSFDYSKFKTFQEMHRKKWTVYTNGKKYIYSKKERKEKQIDVTELMKEELRKVGITEYDNLYSQITNVEDDKEHADFWKSLQFVFDRTMQLRSSQIDNGEDNLEDKIISPVKNAEGVFYESNGNYGDTSQPADADTNGAFHIARKGLLLAENVKKTGRGANGKWNSSVKNISNKDWFAFVQK

SEQ ID NO: 75 - protein, 1323 aa, synthetic construct
MGSGERMYEEFRNCYSVRKTLSFKAIPTEETKKHLQLQWEVLGDEIRFENYDKMKMVLDQLHQSYISRKLDNIGEENQKKIVEILEKLVLVMKKIDTTHQKDKEKAQNQLQSLQASLRKEIGMFFPKNEWQQLQGKNVFKKDGVLSEYNISEENKKNIQCYDGFMTFFKKYNETRANIYSTEEKSTAITFRIVNDNLPKYVRNADNYEQIKKLIPEALEEVEKTYPNLTNYFSIKNYLKYWSQKGIETYNTVIGEINKQVNLVVQQRKDSKFRKYKMQVLYQQILSDREEQSFVYQQDQEVFAAVNELAELVNGSAFNEAIELLKSPNINENEIFIPYAKLAEVSIKMKMGWNGLEEAFINDLQQQYPKKDHEKLVQKLKKEKKVFSLNEIKDVVMKIEHEEDWKFVSLLDCVEDYQKQLTETRDAYVEYAKTYAGSTGTSLQGNDVAPIKAFLDSCLQLVRWCKLFEYSDLYGNRDKIFYGGAESIILALDSLISVYNKTRNYVTMRPGQARKMHLMFNYPEFGDGFSNSKVDSYGTILLREGKKYYLAVIKKGIKVLLEDTINENDSYERLSYMLFPDVKKMIPKCSISTKKVKEHFENSDDDYTIRKGESYAKELLVKKEDYDLYFVNLYDDKKMFQKDYLSKTGDKKGYRQALERWIRFCIRFLQAYKSTKDYDLSELEPISNFRSLDEFYDKLDTLLYKIEWKTISREQIKQMESSGQLFLFELYNKDFSEHAKGKKNLFTLYWEQIFCEENLKQPVIKLCGGAEMFYRKVAIQKKYVHKKDSILVDKTYVDQNGVRKTLPDTIYKEWSDFMNKKITSVSQEASKYKGLVNCHEAKYDITKDKRYTEDQFEFHVPITLNYSALGKGQLNDSVLDCLCQKEKYNVIGIDRGERNLLAYCVVNQDGQILEQGTFNKIVGGNKQEVDYKQKLQEKEVNRQQARKEWKNIGKIKELKNGYLSQVIYQLTQMMVKYDAIVVMEDLNVGFKRGRFKVERQVYQKFEKALIDKLNYLVTKKDENQYGIEGSVSNAYQLTEKIKSFKDIGKQNGMIFYVPAGYTSKIDPTTGFVDVLNRTGLTNAKARKAFFENFDDINYSKEDNMFAFSFDYSKFKTFQEMHRKKWTVYTNGKKYIYSKKERKEKQIDVTELMKEELRKVGITEYDNLYSQITNVEDDKEHADFWKSLQFVFDRTMQLRSSQIDNGEDNLEDKIISPVKNAEGVFYESNGNYGDTSQPADADTNGAFHIARKGLLLAENVKKTGRGANGKWNSSVKNISNKDWFAFVQKSRKRTADGSEFESPKKKRKVGSGKPIPNPLLGLDST

SEQ ID NO: 76 - DNA, 3855 bp, unidentified
  source 1..3855: mol_type = unassigned DNA
atggagagaatgtacgaagaatttagaaattgttattcagtacgaaaaacattgtcatttaaggcaatcccaacagaggaaacaaaaaaacatttacaattacaatgggaagtgttgggggatgagatacgttttgaaaactatgataaaatgaaaatggttttggatcaacttcatcaatcatatatttcgagaaaattagataatataggagaagaaaatcaaaaaaagatagttgaaatcttagagaaactcgtattagttatgaaaaagatagatactacgcatcaaaaggataaagagaaagcgcaaaatcagcttcaatcgttacaagcttcattaaggaaagaaataggaatgttttttcctaaaaacgaatggcaacaattacagggaaaaaatgtatttaagaaggatggggtactaagcgagtataacatttcggaagagaataagaaaaatattcaatgttatgatggttttatgacattctttaaaaaatataatgaaactagagcaaatatatatagtacagaggaaaaaagcacggcaatcacttttcgaattgtgaatgataatcttccaaaatatgtgagaaatgcggataattacgaacagattaaaaaattaattcctgaagctcttgaagaagtagaaaaaacatacccaaatttgacgaattatttctcgattaaaaactatttgaagtattggagtcagaaggggattgaaacatacaatactgttattggagaaataaataagcaggttaatcttgtagtacaacaaagaaaagattcgaaatttagaaaatacaagatgcaggtgttgtatcaacaaattctaagtgatagagaggaacagtcttttgtgtatcaacaggatcaggaagtttttgctgctgttaatgaacttgcagaacttgtgaacggtagtgcttttaacgaggcaattgaattgttgaaatcacctaatattaacgaaaatgagatatttattccctatgcaaaattagcagaagtatccataaaaatgaaaatgggatggaatggattagaggaggcttttataaacgatttgcaacagcagtatccaaagaaggatcatgaaaaattggtgcaaaaattaaaaaaagagaaaaaagttttttctttgaatgaaattaaagatgttgttatgaaaattgaacatgaagaagattggaaatttgttagtttgctggattgtgttgaggattatcaaaaacagttgacagagacaagagatgcatatgtggaatatgcaaaaacttatgcaggttcaaccggtacatcattacaaggaaatgatgtagcaccgataaaagcatttttagatagttgtttgcaattggtacgatggtgtaagttgtttgaatattctgatttgtatggaaatcgagataaaatattttatggaggagcagagtcgattatacttgcattagattccttaatatctgtgtataataaaacaagaaattatgtgactatgcgaccggggcaggctagaaaaatgcatttaatgtttaattatccggaattcggtgatggctttagtaatagtaaagtggattcttatggtacgattttgcttcgtgaaggaaagaaatattatttagctgttattaaaaaaggcataaaagtcttgctggaagataccataaatgaaaatgacagttatgaacgtttgagttatatgttgtttcctgatgtaaaaaaaatgataccgaaatgttctattagtacgaagaaagttaaagaacattttgaaaattcggatgatgattatacgattcgtaaaggtgaatcttatgcaaaagaattacttgtgaaaaaagaagattatgacctttactttgtaaatctttatgatgataagaagatgtttcaaaaggactatttgagtaaaactggagataaaaaaggatatagacaggcgttagaacgctggatacgtttttgcattcgatttttacaagcttataagagtacaaaggattatgatctcagtgaattagagccaatttcgaattttcgttccttagatgagttttatgataaattggatactttgttatacaagatagagtggaaaacaatttcaagagaacaaattaagcaaatggagtcatctggtcagttgtttttatttgaattatataacaaagatttctctgaacatgcaaaaggaaagaaaaatttatttacattgtattgggaacagattttctgtgaagagaatttaaaacagccagtgattaaactttgtggcggggcagagatgttttatcgtaaggttgccattcaaaaaaaatatgtacataaaaaagactccattttggtggataaaacgtatgtggatcagaatggagtcagaaaaacacttccggatactatatataaagagtggtcggattttatgaataaaaagataacatctgtcagccaggaggcaagtaaatataaaggtttggttaattgtcatgaggcaaaatatgatattacaaaagataaaagatatacggaagatcaatttgagtttcatgtgccaattactttaaattattcagcattaggaaaagggcaattaaatgatagtgttctggattgtctatgtcagaaagaaaaatataatgtgataggaattgaccgtggagaaagaaacttgttggcttactgtgtcgtaaatcaagatggacagattttagaacaagggacatttaataagattgtaggtggaaataaacaggaagtagattacaaacagaagttacaggagaaagaagtaaatcgacaacaagcaagaaaagagtggaaaaatattggaaaaattaaagaattaaagaacggttatttgtctcaggttatttatcaactgacgcaaatgatggtaaaatatgatgctattgttgttatggaagatttgaatgttggctttaaacgtggtcgatttaaggtggaacgacaggtttaccagaaatttgaaaaagcgctgattgacaaattaaattatttagtaactaaaaaagatgaaaatcaatatggaatagagggtagcgtaagcaatgcatatcaactgacagaaaaaatcaaatcatttaaagatattggcaaacaaaacgggatgatattttatgtgccagcgggatatacctctaaaatagatcctacaacaggatttgtggatgtgctaaatcgaacaggattaacaaatgccaaagccagaaaagcgttctttgaaaattttgatgatattaactattcaaaagaagataatatgtttgccttttcttttgattatagcaagtttaagacatttcaagaaatgcatagaaaaaaatggacagtttacacaaatggtaaaaagtacatttattcaaaaaaagaacgaaaagaaaaacaaattgatgttactgagttgatgaaagaagaattgagaaaagtaggaattacagagtatgataatctttattcgcaaattactaatgtggaagatgataaagaacatgcagatttttggaaatctttacagtttgtatttgatagaacgatgcagttgagaagtagtcaaattgacaatggagaggataatcttgaggataagattatatctccggtgaaaaatgcagagggtgtattttatgaatcaaatggaaattatggtgacacttcacaacctgcagatgcagatacaaatggtgcttttcatattgcaaggaagggattactacttgcagaaaatgtgaaaaaaacaggtagaggagcaaatggaaaatggaattcttctgtaaaaaatatttctaataaggattggtttgcatttgttcaaaaataa

SEQ ID NO: 77 - DNA, 3849 bp, synthetic construct
  source 1..3849: mol_type = other DNA
gaacggatgtacgaggagttcagaaactgctactccgtgcggaaaacactgtccttcaaagccatccctaccgaggagacaaagaagcacctgcagctgcagtgggaagtgctcggcgacgagattagatttgagaattatgataagatgaaaatggtgctggaccagctgcaccagtcttacatcagccggaagctggacaacatcggcgaggagaaccagaaaaagattgtagaaatcctggagaagctggtgctggtgatgaagaagatcgatacaacccaccagaaggacaaggagaaggcccagaatcaactgcagagcctgcaggcttccctgcggaaggaaattggtatgtttttcccaaagaacgagtggcagcagctgcagggcaaaaacgtgttcaagaaggacggcgttctcagcgaatacaacatcagcgaggaaaacaagaagaacatccagtgttacgacggctttatgaccttcttcaagaagtacaacgagacacgggccaatatctattctacggaggaaaagagcaccgccatcaccttcaggatcgtgaatgataatctgcctaagtatgtgcgaaacgctgacaactacgagcagataaagaagctgatccccgaagctctggaagaagtcgaaaagacctatcctaatctgaccaactacttcagcatcaagaactatctgaagtactggagccagaaggggatcgaaacatacaacaccgtgatcggcgagatcaacaagcaggtgaacctggtggtccaacagagaaaggacagcaagttcaggaagtacaaaatgcaggtgctgtaccagcagatcctatccgacagagaggagcagagcttcgtgtaccagcaggaccaggaggtgttcgccgccgtgaacgagctggccgagctggtgaatggcagcgccttcaatgaagctatcgaattgctgaaaagcccaaacatcaacgagaatgagattttcatcccctacgccaagctcgccgaggtgtctatcaagatgaaaatgggatggaacggcctggaggaggccttcatcaacgatctgcagcaacaataccccaagaaagaccacgaaaaattggttcagaagctgaagaaagagaagaaggtgtttagcctgaatgaaatcaaggatgtggtcatgaagatcgaacacgaggaagattggaaattcgtgagcctgctggactgcgtggaggattaccagaagcagcttacagagacaagagatgcctacgtggagtacgctaagacatacgccggcagcacaggcaccagcctgcagggcaacgacgtggcccctatcaaggccttcctggactcctgcctgcaactggtgcggtggtgcaagctgttcgagtacagcgacctgtacggcaacagagacaagatcttctacggaggcgccgagagcatcatcctggccctggatagcctgatttccgtgtacaacaaaaccagaaactacgtgaccatgcggcctggccaggccagaaaaatgcacctgatgttcaactaccccgagtttggcgacggcttcagcaacagcaaagtggattcttacggcaccatcctgctgagagaaggcaagaagtactacctggctgtgatcaagaagggcatcaaagtgctgctggaggacaccattaacgagaatgactcttacgagcggctgtcctacatgctgttccccgacgtgaaaaagatgatccctaagtgcagcatcagtaccaagaaggtgaaagagcatttcgagaacagcgacgacgactacaccatcagaaagggcgagagctatgccaaggagctgctggtgaagaaggaagattacgacctgtatttcgtgaacctgtacgacgacaaaaagatgttccagaaagactacctgagcaaaaccggcgacaagaagggatacagacaggccctggagaggtggatcagattctgcatcagattcctgcaggcttacaagtctacaaaggattatgacctgtctgaactggaacctatcagcaacttcagaagcctggacgagttctacgataagctggacaccctactgtacaagatcgagtggaaaaccatctccagagagcagatcaagcaaatggaatcctctggccagctcttcctgttcgagttgtacaacaaggacttctctgaacacgccaagggaaagaagaacctgttcaccctgtactgggagcaaattttttgtgaagagaacctgaagcagcctgtgatcaagctgtgcggcggagccgagatgttctacagaaaggttgccatccagaaaaagtacgtgcacaagaaggacagcatcctggtagacaagacctacgtggatcagaacggcgttcgcaagaccctgcctgataccatctacaaggaatggtccgacttcatgaacaaaaagatcaccagcgtgtcccaagaagcctctaaatacaagggcctggtgaactgtcacgaggccaagtacgacatcaccaaggacaagagatacaccgaagatcaattcgaatttcacgtgccaatcacactgaactacagcgccctcggaaaaggtcagctgaacgacagcgtgctggactgcctgtgtcagaaagagaagtacaacgtgattggaatcgaccggggagaaagaaacctgctggcctactgcgtggtgaaccaggatggccagatcctggaacagggcaccttcaacaagatcgtgggcggcaataagcaggaggtggactataagcagaaactgcaggagaaggaggtgaatagacagcaggccaggaaggagtggaagaacatcggcaagatcaaggagttgaaaaacggctacctgagccaagtaatctaccagctgacacagatgatggtgaagtacgatgccatcgtggtgatggaagatctgaacgtgggctttaagagaggcagattcaaggttgagcggcaggtgtaccagaagttcgaaaaggctctgatcgataagctgaattatctggtcaccaagaaggacgagaaccaatacgggatcgagggcagcgtttcgaatgcctaccagctgaccgagaaaatcaagagcttcaaagacatcggaaaacagaacggcatgatcttctacgtgcctgctggctatacaagcaaaatcgaccctacgaccggattcgtcgatgtgctgaacagaaccggcctgacaaacgccaaggctagaaaagccttcttcgagaattttgacgacatcaactactctaaggaggacaacatgttcgccttcagcttcgattacagcaagttcaagacctttcaggaaatgcatagaaaaaagtggacagtgtacacaaacggaaaaaaatacatctacagcaagaaggaacggaaggaaaagcagatagacgtgaccgaactgatgaaagaagagctgagaaaggtgggcataaccgagtacgacaacctctacagccagatcaccaacgtggaagatgataaggagcacgccgacttttggaagtctctgcagttcgtgttcgacagaacaatgcagctgagaagcagccagatcgacaacggcgaggacaatctggaagataagatcatttcacctgtgaaaaacgccgagggcgtgttctatgaaagcaacggcaactacggcgatacgagccagcccgccgacgcggacaccaacggcgccttccacatcgcgcggaagggcctgctgctcgccgagaatgtgaagaaaaccggaagaggcgccaatggcaaatggaatagcagcgtgaagaacatctctaacaaggattggttcgcctttgtgcagaaa

SEQ ID NO: 78 - DNA, 3972 bp, synthetic construct
  source 1..3972: mol_type = other DNA
atgggctccggagaacggatgtacgaggagttcagaaactgctactccgtgcggaaaacactgtccttcaaagccatccctaccgaggagacaaagaagcacctgcagctgcagtgggaagtgctcggcgacgagattagatttgagaattatgataagatgaaaatggtgctggaccagctgcaccagtcttacatcagccggaagctggacaacatcggcgaggagaaccagaaaaagattgtagaaatcctggagaagctggtgctggtgatgaagaagatcgatacaacccaccagaaggacaaggagaaggcccagaatcaactgcagagcctgcaggcttccctgcggaaggaaattggtatgtttttcccaaagaacgagtggcagcagctgcagggcaaaaacgtgttcaagaaggacggcgttctcagcgaatacaacatcagcgaggaaaacaagaagaacatccagtgttacgacggctttatgaccttcttcaagaagtacaacgagacacgggccaatatctattctacggaggaaaagagcaccgccatcaccttcaggatcgtgaatgataatctgcctaagtatgtgcgaaacgctgacaactacgagcagataaagaagctgatccccgaagctctggaagaagtcgaaaagacctatcctaatctgaccaactacttcagcatcaagaactatctgaagtactggagccagaaggggatcgaaacatacaacaccgtgatcggcgagatcaacaagcaggtgaacctggtggtccaacagagaaaggacagcaagttcaggaagtacaaaatgcaggtgctgtaccagcagatcctatccgacagagaggagcagagcttcgtgtaccagcaggaccaggaggtgttcgccgccgtgaacgagctggccgagctggtgaatggcagcgccttcaatgaagctatcgaattgctgaaaagcccaaacatcaacgagaatgagattttcatcccctacgccaagctcgccgaggtgtctatcaagatgaaaatgggatggaacggcctggaggaggccttcatcaacgatctgcagcaacaataccccaagaaagaccacgaaaaattggttcagaagctgaagaaagagaagaaggtgtttagcctgaatgaaatcaaggatgtggtcatgaagatcgaacacgaggaagattggaaattcgtgagcctgctggactgcgtggaggattaccagaagcagcttacagagacaagagatgcctacgtggagtacgctaagacatacgccggcagcacaggcaccagcctgcagggcaacgacgtggcccctatcaaggccttcctggactcctgcctgcaactggtgcggtggtgcaagctgttcgagtacagcgacctgtacggcaacagagacaagatcttctacggaggcgccgagagcatcatcctggccctggatagcctgatttccgtgtacaacaaaaccagaaactacgtgaccatgcggcctggccaggccagaaaaatgcacctgatgttcaactaccccgagtttggcgacggcttcagcaacagcaaagtggattcttacggcaccatcctgctgagagaaggcaagaagtactacctggctgtgatcaagaagggcatcaaagtgctgctggaggacaccattaacgagaatgactcttacgagcggctgtcctacatgctgttccccgacgtgaaaaagatgatccctaagtgcagcatcagtaccaagaaggtgaaagagcatttcgagaacagcgacgacgactacaccatcagaaagggcgagagctatgccaaggagctgctggtgaagaaggaagattacgacctgtatttcgtgaacctgtacgacgacaaaaagatgttccagaaagactacctgagcaaaaccggcgacaagaagggatacagacaggccctggagaggtggatcagattctgcatcagattcctgcaggcttacaagtctacaaaggattatgacctgtctgaactggaacctatcagcaacttcagaagcctggacgagttctacgataagctggacaccctactgtacaagatcgagtggaaaaccatctccagagagcagatcaagcaaatggaatcctctggccagctcttcctgttcgagttgtacaacaaggacttctctgaacacgccaagggaaagaagaacctgttcaccctgtactgggagcaaattttttgtgaagagaacctgaagcagcctgtgatcaagctgtgcggcggagccgagatgttctacagaaaggttgccatccagaaaaagtacgtgcacaagaaggacagcatcctggtagacaagacctacgtggatcagaacggcgttcgcaagaccctgcctgataccatctacaaggaatggtccgacttcatgaacaaaaagatcaccagcgtgtcccaagaagcctctaaatacaagggcctggtgaactgtcacgaggccaagtacgacatcaccaaggacaagagatacaccgaagatcaattcgaatttcacgtgccaatcacactgaactacagcgccctcggaaaaggtcagctgaacgacagcgtgctggactgcctgtgtcagaaagagaagtacaacgtgattggaatcgaccggggagaaagaaacctgctggcctactgcgtggtgaaccaggatggccagatcctggaacagggcaccttcaacaagatcgtgggcggcaataagcaggaggtggactataagcagaaactgcaggagaaggaggtgaatagacagcaggccaggaaggagtggaagaacatcggcaagatcaaggagttgaaaaacggctacctgagccaagtaatctaccagctgacacagatgatggtgaagtacgatgccatcgtggtgatggaagatctgaacgtgggctttaagagaggcagattcaaggttgagcggcaggtgtaccagaagttcgaaaaggctctgatcgataagctgaattatctggtcaccaagaaggacgagaaccaatacgggatcgagggcagcgtttcgaatgcctaccagctgaccgagaaaatcaagagcttcaaagacatcggaaaacagaacggcatgatcttctacgtgcctgctggctatacaagcaaaatcgaccctacgaccggattcgtcgatgtgctgaacagaaccggcctgacaaacgccaaggctagaaaagccttcttcgagaattttgacgacatcaactactctaaggaggacaacatgttcgccttcagcttcgattacagcaagttcaagacctttcaggaaatgcatagaaaaaagtggacagtgtacacaaacggaaaaaaatacatctacagcaagaaggaacggaaggaaaagcagatagacgtgaccgaactgatgaaagaagagctgagaaaggtgggcataaccgagtacgacaacctctacagccagatcaccaacgtggaagatgataaggagcacgccgacttttggaagtctctgcagttcgtgttcgacagaacaatgcagctgagaagcagccagatcgacaacggcgaggacaatctggaagataagatcatttcacctgtgaaaaacgccgagggcgtgttctatgaaagcaacggcaactacggcgatacgagccagcccgccgacgcggacaccaacggcgccttccacatcgcgcggaagggcctgctgctcgccgagaatgtgaagaaaaccggaagaggcgccaatggcaaatggaatagcagcgtgaagaacatctctaacaaggattggttcgcctttgtgcagaaatctagaaagcggacagcagacggctccgaatttgaaagccctaagaaaaagagaaaggtgggatccggcaaacctatccccaatcccctgctgggcctggacagcacctga

SEQ ID NO: 79 - protein, 1288 aa, unidentified
SFESFTNVYPVSKTLRFELRPVGATAEKLKESGILEHDTKRGKEYATLKDLLDEQHKELLADALKPERVKNALKPNSGKSKKDKLVEENYITEDGEIRWETLAAAMEAFRAGEVEKNVLEAIQTQFRKLIVTILKADERYPGLTASTPSAVIKTLLKQDVHPEAVETFAKFACYFTGFQENRKNIYAEEKQATAVATRVVHDNFAKFHTQSKIIGVIKNKYPEILQSVEMELMDELGGMKITDIFSINSYSKWMTQEGIDFINKIIGGYSPSVGVKVRGLNEFINLYRQQHEEANADRRNLAKMPMLFKQILSDISTRSFIPVMFENDAELKDSIEAFLTGLNDFELNAQKFNVVVALGNLFQKIVPCEGIFLDAALMEKVSKTATGDWSLLAQSMEAYAETAFTRAKDRDAWLRKNYYSLSELSQVPILKNTDEGMLKFELSAYWSGEKMESFVKGIMDAELAMKPVLASIGQKTEEVRLRDRIDDVVKIKGYLDSIQNFLHHLKPFCAPTELNRDADFYSDFDALYNQLVLVIPLYNCVRNYVTQKVTEVQKLRLKFDAPTLADGWDANKENDNKAVLFEKDGLYFLGILNPNLKAKDRPVFEHESNVTKKSCYRKIVYKLLPGPNKMLPKVFFADSNRTLYHPSKSLLDRYHNGEYKKGDSFDIKFCHELIDYFKASISIHPDWKEFGFQFSATKTYESIDGFYREVEEQGYKVNFAFVRADLIDKYVESGSLFLFQLYNKDFSCASSGKPNLHTLYWKSLFAKENLDEPILKLCGGAELFFRPVAIQKPYVHTLGEKLVNRRLGEHGKGEAIPERVHKELVDYYNHRVSVLSHDGKAFKDKVVVRDVAHSITKDRRFSEAKFFFHVPIMFNRTASKSAKFNDKVVDYLKTTQNVNVIGLDRGERNLIYLTMVNLHGKLIEQRSFNLVNGVDYHSKLDLREKERMDARVNWENIGGIKDLKTGYLSAVVHEIAKMMVTNNAIVVLEDLNFGFKRGRFKVEKQVYQKFEKMLIDKLNFLMFKECNQAALGGVRRAYQLTDKFVSFEKLGKQTGFLFYVPAGYTSKIDPTTGFTNLFNTKKCTNAEGRKVFFEAMNSIIYDGSRKSFAFSFDYGNPVFRASQTSFKKEWTVYSADTRIVYNRGEKTVNTIHPTQILHDALCALGIDVHDGLNVLNVVRETPADKIHAKFFSDLFYAFDRTLQMRNSVSGTDEDYIQSPVLNATGEFFDSRKADSTLPQDADANGAYHIALKGLLLLQRMKDIGSDIKLDLSIKHEDWFAFAQKRCQR

SEQ ID NO: 80 - protein, 1289 aa, unidentified
MSFESFTNVYPVSKTLRFELRPVGATAEKLKESGILEHDTKRGKEYATLKDLLDEQHKELLADALKPERVKNALKPNSGKSKKDKLVEENYITEDGEIRWETLAAAMEAFRAGEVEKNVLEAIQTQFRKLIVTILKADERYPGLTASTPSAVIKTLLKQDVHPEAVETFAKFACYFTGFQENRKNIYAEEKQATAVATRVVHDNFAKFHTQSKIIGVIKNKYPEILQSVEMELMDELGGMKITDIFSINSYSKWMTQEGIDFINKIIGGYSPSVGVKVRGLNEFINLYRQQHEEANADRRNLAKMPMLFKQILSDISTRSFIPVMFENDAELKDSIEAFLTGLNDFELNAQKFNVVVALGNLFQKIVPCEGIFLDAALMEKVSKTATGDWSLLAQSMEAYAETAFTRAKDRDAWLRKNYYSLSELSQVPILKNTDEGMLKFELSAYWSGEKMESFVKGIMDAELAMKPVLASIGQKTEEVRLRDRIDDVVKIKGYLDSIQNFLHHLKPFCAPTELNRDADFYSDFDALYNQLVLVIPLYNCVRNYVTQKVTEVQKLRLKFDAPTLADGWDANKENDNKAVLFEKDGLYFLGILNPNLKAKDRPVFEHESNVTKKSCYRKIVYKLLPGPNKMLPKVFFADSNRTLYHPSKSLLDRYHNGEYKKGDSFDIKFCHELIDYFKASISIHPDWKEFGFQFSATKTYESIDGFYREVEEQGYKVNFAFVRADLIDKYVESGSLFLFQLYNKDFSCASSGKPNLHTLYWKSLFAKENLDEPILKLCGGAELFFRPVAIQKPYVHTLGEKLVNRRLGEHGKGEAIPERVHKELVDYYNHRVSVLSHDGKAFKDKVVVRDVAHSITKDRRFSEAKFFFHVPIMFNRTASKSAKFNDKVVDYLKTTQNVNVIGLDRGERNLIYLTMVNLHGKLIEQRSFNLVNGVDYHSKLDLREKERMDARVNWENIGGIKDLKTGYLSAVVHEIAKMMVTNNAIVVLEDLNFGFKRGRFKVEKQVYQKFEKMLIDKLNFLMFKECNQAALGGVRRAYQLTDKFVSFEKLGKQTGFLFYVPAGYTSKIDPTTGFTNLFNTKKCTNAEGRKVFFEAMNSIIYDGSRKSFAFSFDYGNPVFRASQTSFKKEWTVYSADTRIVYNRGEKTVNTIHPTQILHDALCALGIDVHDGLNVLNVVRETPADKIHAKFFSDLFYAFDRTLQMRNSVSGTDEDYIQSPVLNATGEFFDSRKADSTLPQDADANGAYHIALKGLLLLQRMKDIGSDIKLDLSIKHEDWFAFAQKRCQR

SEQ ID NO: 81 - protein, 1328 aa, synthetic construct
MGSGSFESFTNVYPVSKTLRFELRPVGATAEKLKESGILEHDTKRGKEYATLKDLLDEQHKELLADALKPERVKNALKPNSGKSKKDKLVEENYITEDGEIRWETLAAAMEAFRAGEVEKNVLEAIQTQFRKLIVTILKADERYPGLTASTPSAVIKTLLKQDVHPEAVETFAKFACYFTGFQENRKNIYAEEKQATAVATRVVHDNFAKFHTQSKIIGVIKNKYPEILQSVEMELMDELGGMKITDIFSINSYSKWMTQEGIDFINKIIGGYSPSVGVKVRGLNEFINLYRQQHEEANADRRNLAKMPMLFKQILSDISTRSFIPVMFENDAELKDSIEAFLTGLNDFELNAQKFNVVVALGNLFQKIVPCEGIFLDAALMEKVSKTATGDWSLLAQSMEAYAETAFTRAKDRDAWLRKNYYSLSELSQVPILKNTDEGMLKFELSAYWSGEKMESFVKGIMDAELAMKPVLASIGQKTEEVRLRDRIDDVVKIKGYLDSIQNFLHHLKPFCAPTELNRDADFYSDFDALYNQLVLVIPLYNCVRNYVTQKVTEVQKLRLKFDAPTLADGWDANKENDNKAVLFEKDGLYFLGILNPNLKAKDRPVFEHESNVTKKSCYRKIVYKLLPGPNKMLPKVFFADSNRTLYHPSKSLLDRYHNGEYKKGDSFDIKFCHELIDYFKASISIHPDWKEFGFQFSATKTYESIDGFYREVEEQGYKVNFAFVRADLIDKYVESGSLFLFQLYNKDFSCASSGKPNLHTLYWKSLFAKENLDEPILKLCGGAELFFRPVAIQKPYVHTLGEKLVNRRLGEHGKGEAIPERVHKELVDYYNHRVSVLSHDGKAFKDKVVVRDVAHSITKDRRFSEAKFFFHVPIMFNRTASKSAKFNDKVVDYLKTTQNVNVIGLDRGERNLIYLTMVNLHGKLIEQRSFNLVNGVDYHSKLDLREKERMDARVNWENIGGIKDLKTGYLSAVVHEIAKMMVTNNAIVVLEDLNFGFKRGRFKVEKQVYQKFEKMLIDKLNFLMFKECNQAALGGVRRAYQLTDKFVSFEKLGKQTGFLFYVPAGYTSKIDPTTGFTNLFNTKKCTNAEGRKVFFEAMNSIIYDGSRKSFAFSFDYGNPVFRASQTSFKKEWTVYSADTRIVYNRGEKTVNTIHPTQILHDALCALGIDVHDGLNVLNVVRETPADKIHAKFFSDLFYAFDRTLQMRNSVSGTDEDYIQSPVLNATGEFFDSRKADSTLPQDADANGAYHIALKGLLLLQRMKDIGSDIKLDLSIKHEDWFAFAQKRCQRSRKRTADGSEFESPKKKRKVGSGKPIPNPLLGLDST

SEQ ID NO: 82 - DNA, 3870 bp, unidentified
  source 1..3870: mol_type = unassigned DNA
atgagttttgaatcattcactaacgtttatcccgtttccaagactttgcgctttgagctgaggcccgttggtgcaactgcagagaagcttaaggaaagtggtatccttgagcatgatacgaaacgaggtaaggaatatgcgactctcaaggatctgcttgatgagcaacataaggagttacttgctgacgccctaaaacctgaacgtgtgaagaatgcgcttaagcccaatagtggtaagagtaaaaaagataaattggttgaagagaattacattacggaagacggggagattcgatgggaaactcttgcggctgcgatggaggcatttcgcgccggtgaggtagagaaaaatgtgcttgaagcaatacagacgcaatttagaaagctgattgtaacgatactgaaggcggatgagcggtatccgggactgacagcttcaacgccttcggctgtcattaagactcttcttaagcaggatgttcatccagaagcagtagagacatttgcaaaatttgcctgttattttaccggttttcaggaaaatcggaagaatatctatgcggaagaaaagcaagcaactgcagttgcaacgcgagttgttcatgataatttcgcaaagttccatacacaatcgaaaataataggtgtcataaagaataaatatccagaaatccttcagtcggtagaaatggaattgatggacgaattaggtgggatgaaaatcactgatatcttttctatcaacagctattccaaatggatgacgcaagaagggatagactttattaataagattataggtggctatagcccatctgttggtgtgaaggtgcgtggtctgaacgagttcattaatctttatcggcagcagcatgaagaggcaaatgcagatcggcggaatctcgcaaaaatgccgatgctgtttaaacaaattttaagtgatatttcgacacgatcattcattccggtgatgtttgaaaatgatgcggaactaaaggattcaatagaagcattcttgacaggtctgaatgattttgagttgaatgctcagaagtttaacgttgtcgttgcattaggtaatcttttccaaaaaattgtgccttgcgaaggtattttcttggatgcagcattgatggaaaaagtttcgaagacggctacaggagattggagtcttcttgctcagtcgatggaggcgtatgcagagacagcattcacaagagcaaaagaccgagacgcatggctaaggaaaaattattattcgctgtccgagctgagccaagttccgattttgaagaacactgatgaaggaatgttgaagtttgaactatctgcctattggtcaggcgaaaagatggaaagttttgttaaaggaatcatggatgctgaattggcaatgaaaccagttcttgccagcattggtcagaaaaccgaagaggtgcgtcttcgtgatcggattgacgatgtcgtaaaaatcaagggatatcttgattcaattcagaattttttacatcacctaaaaccgttttgtgctccaactgaattgaatcgtgatgcggatttttattctgactttgacgcattgtataatcagcttgtactggttataccgctttataactgtgtccgcaattacgtgacacagaaagtgacagaggttcagaaactgaggctaaagtttgatgcccctacattggcggacggatgggacgcgaataaagaaaatgataataaggcagttctgtttgaaaaggacgggctatattttcttggaatcctgaatcctaacctgaaggcgaaagatcgtccagtctttgagcatgaaagtaatgttacaaagaaatcttgttatcgcaagattgtctataaacttttgccaggaccaaataaaatgcttcccaaggtcttttttgctgattccaataggacactgtaccatccttccaagtcgttgctggatcgttatcacaacggtgaatacaagaaaggcgattcattcgacatcaaattctgtcatgaattgattgattattttaaagcctcgattagtattcaccccgattggaaggaattcggtttccaattcagtgcgacaaaaacatatgagagcattgatggtttttatcgtgaggttgaggagcaaggatataaagttaattttgcttttgtaagggcggatttaattgacaaatatgtggaaagtggaagtttgttccttttccaattgtataacaaggatttctcttgtgcgtcatctgggaagccaaacctccacacgctttactggaagagcctctttgcaaaagaaaaccttgatgagccgattctgaagttgtgtgggggtgcagagctattcttccgcccagttgcaatccagaagccgtatgtacataccttgggagaaaagttggtcaatcgcaggcttggcgagcacggtaagggagaggcaatcccggagagagttcacaaggaactcgtggactactacaaccatcgtgtgtcggtgctgagtcatgatgggaaggcatttaaagacaaggttgttgttcgggatgtcgcacattcgattacaaaagatcgtcgattctcagaggcaaagttttttttccatgttccgatcatgtttaaccgtacagcatcgaagagtgcaaagtttaacgacaaagttgtggactatctcaagaccactcagaatgtaaacgttatcgggttggatcgaggagaaagaaatctgatttatctgacaatggtaaatttgcacggaaagctgatagagcagcgtagtttcaacctagttaatggtgtggattatcattcaaagctagatttgcgagaaaaggagcgcatggacgcacgcgttaattgggagaacattgggggaattaaagatcttaagaccggatatctttccgcggttgttcatgagattgcgaagatgatggtgacgaataatgccattgttgtcttggaggacttgaacttcggtttcaaacgtgggcggttcaaggttgagaaacaggtctatcagaagtttgagaagatgctgattgataaactgaatttcctgatgttcaaggaatgcaatcaagcggctctcggtggtgttcgccgtgcatatcaattgacggataaattcgtgagttttgaaaaacttggtaaacaaacgggtttcctgttttatgttccggcgggctacacatcgaagattgatccaacaactggattcaccaacctcttcaacacgaaaaaatgcactaatgccgaaggtcggaaggtcttctttgaggcgatgaactctatcatatatgacggatcaaggaagtcgtttgcgttctcatttgattacggcaacccagtttttagagcaagtcaaacgagttttaaaaaagaatggaccgtctattccgctgatacgcgcattgtctacaatcgtggcgagaaaactgttaatacgatccatccgacacaaattcttcatgatgctttgtgtgcactcggcattgacgttcatgacggattgaacgtcttgaacgtagttcgtgagacgccagcggacaagattcatgctaagtttttctcagacttgttctatgcgtttgatcgtacacttcagatgcgtaacagtgtttcaggaacagatgaagactatatccaatcgcctgttttgaatgcgacaggtgagttttttgattcgcggaaagcagacagtactcttccgcaggatgccgatgccaatggtgcctaccacatcgcattaaagggacttttgctgctacaacgcatgaaagatattggcagtgatatcaagcttgatctatccattaagcatgaggactggtttgcgtttgcacaaaagcgttgccagagataa

SEQ ID NO: 83 - DNA, 3864 bp, synthetic construct
  source 1..3864: mol_type = other DNA
agcttcgagtctttcactaacgtataccctgtgtctaaaaccctgcgttttgaactgcggcctgtgggcgccactgccgagaagctgaaggagagcggcatcctggagcacgataccaagcggggcaaggaatacgctacactgaaggacctgctggacgagcagcacaaagagctactggccgacgccctgaagccagagagagtgaagaacgccctgaagcccaacagcggcaagtccaaaaaggacaagctggtcgaagagaactacattacagaagatggagagatcagatgggagacactggccgctgctatggaggccttcagagctggcgaagtggagaagaacgtgctggaagcgatccagacacagtttcggaagctgatcgtgaccatcctgaaagccgacgagagataccctggactgaccgcctctacacctagcgccgtcatcaagaccttgctgaagcaggacgtgcaccccgaggccgtagagacattcgctaaatttgcctgttacttcaccggctttcaggaaaacagaaagaatatctacgccgaagaaaaacaggccaccgccgtggccacacgggttgtccacgacaacttcgccaaatttcacacccagtctaagattatcggcgtgatcaaaaacaagtaccccgagatcctgcagagcgtcgagatggaactgatggacgaacttgggggaatgaagatcaccgatatcttcagtatcaacagctacagcaagtggatgacccaggagggaatcgacttcatcaacaaaatcatcggcggctacagccctagcgtgggcgtcaaagtgagaggcctgaacgagttcatcaacctgtacagacagcagcacgaggaagccaacgccgaccggcggaacctggctaagatgcctatgctgtttaaacaaattctgagcgacatcagcacccggagcttcatccctgtgatgttcgagaatgacgccgagctcaaggacagcatcgaggccttcctgacaggcctgaatgatttcgagctgaacgctcagaagttcaacgttgtggtggccctggggaacctgtttcagaagattgtgccttgtgaaggcatcttcctggacgctgccctgatggaaaaggtttccaagacagctacaggcgactggagcctgctcgcacagtctatggaagcctacgccgaaacagcctttacaagagccaaggaccgggacgcctggctgagaaagaattactacagcctgtccgagctgagccaggtgccaatcctgaagaacactgatgagggcatgctgaagttcgagctgagcgcctactggtccggcgagaaaatggaatctttcgtgaagggcatcatggacgccgagctggccatgaagccagtgctggccagcatcggccagaaaaccgaagaggtgcggctgagagatagaatcgacgacgtggtgaagatcaagggctacctggacagcatccagaatttcctgcaccacctgaagcctttctgtgcccctaccgagctgaaccgggacgccgacttctactctgacttcgatgctctgtacaatcaactggtgctggtgattcccctgtacaactgcgtgagaaactacgtcacccaaaaggttaccgaggtgcagaagctgcgcctcaagttcgatgcacctaccctggccgatggatgggacgccaataaagagaatgacaacaaagccgtcctgttcgagaaagacggcctgtatttcctcggcatcctcaaccctaacctgaaagccaaggaccggcctgtgttcgaacatgaaagcaacgtgaccaagaagtcatgctaccggaagattgtgtacaaactgctgccaggccctaacaagatgctgcctaaggtgttctttgccgatagcaacaggacactgtaccaccctagcaagagcctgctggaccggtatcacaacggcgagtacaagaagggcgatagctttgatatcaagttttgccacgagctgatcgactacttcaaggcctctatctctattcaccctgactggaaggagttcggctttcaattttctgccacaaagacctacgagtctatcgacggcttctatagagaggtggaagagcagggctacaaggtgaacttcgcctttgtgcgtgctgacctgatcgataagtacgtggaaagcggctccctgttcctgttccagctctataacaaggacttcagctgtgcctctagcggcaagccgaatcttcatacactgtactggaaaagcctgttcgccaaggagaacctggacgagcctatactgaagctgtgcggcggcgccgagctgttcttcagacccgtggcgatccagaaaccctacgtgcacacattgggcgaaaagctggtgaatagacggctcggcgagcacggcaagggcgaggctatccctgagcgggtgcacaaggaactggtggactactacaaccacagagtgagcgtgctcagtcacgatggaaaggccttcaaggacaaggtggtggttcgggacgtggcccacagcatcaccaaggaccgacggtttagcgaggccaagttcttcttccacgtgcccatcatgtttaaccggaccgccagcaagagcgccaagttcaacgacaaggtggtggactacctgaaaaccacccaaaacgtgaacgtgatcggactggacagaggtgaaagaaacctgatctacctcacaatggtgaacctgcatggcaagctcatcgagcagcggagcttcaacctggtgaatggcgtggactaccattctaagctggatctgcgcgagaaggaacgtatggatgctagagtgaactgggagaatatcggcggcataaaggatctgaaaaccggctacctgagcgccgtggtgcacgagatcgccaaaatgatggtgacaaacaacgccatcgtggtgctggaagatctgaactttggattcaagagaggcagattcaaagtggaaaagcaggtgtaccagaaattcgagaagatgctgatcgacaaactgaacttcctgatgttcaaagagtgcaaccaggccgccctgggcggcgtgcggcgggcctatcagctgaccgacaagttcgtgagcttcgagaagctgggaaagcagaccggcttcctgttctatgtgcccgccggctatacaagcaaaatcgatcctacaaccggtttcaccaacctgttcaataccaagaaatgcaccaacgccgagggaagaaaggtgttcttcgaggctatgaacagcatcatctacgacggctccagaaaatctttcgcctttagcttcgactacggcaaccccgtgtttcgagcctcccagaccagcttcaagaaggaatggaccgtgtacagcgccgatacaagaatcgtgtataatcggggcgaaaagaccgtaaacaccatccaccctacccagatcctgcacgacgccctgtgcgccttgggaatcgacgtgcacgatgggttaaatgtcttgaacgtcgtgagagagacacccgctgataagatccacgccaagttcttcagcgatctcttctacgccttcgacagaaccctgcagatgaggaactctgtgagcgggaccgacgaagattacatccagagccctgtgctgaatgctaccggcgagttctttgacagcagaaaagccgacagcaccctgccccaggacgcagacgctaatggagcctaccacatcgccctgaagggcctgctgctcctgcagagaatgaaggatatcggctcagatatcaagctggatctgtctattaagcacgaggattggttcgccttcgctcagaagcggtgccagaga

SEQ ID NO: 84 - DNA, 3987 bp, synthetic construct
  source 1..3987: mol_type = other DNA
atgggctccggaagcttcgagtctttcactaacgtataccctgtgtctaaaaccctgcgttttgaactgcggcctgtgggcgccactgccgagaagctgaaggagagcggcatcctggagcacgataccaagcggggcaaggaatacgctacactgaaggacctgctggacgagcagcacaaagagctactggccgacgccctgaagccagagagagtgaagaacgccctgaagcccaacagcggcaagtccaaaaaggacaagctggtcgaagagaactacattacagaagatggagagatcagatgggagacactggccgctgctatggaggccttcagagctggcgaagtggagaagaacgtgctggaagcgatccagacacagtttcggaagctgatcgtgaccatcctgaaagccgacgagagataccctggactgaccgcctctacacctagcgccgtcatcaagaccttgctgaagcaggacgtgcaccccgaggccgtagagacattcgctaaatttgcctgttacttcaccggctttcaggaaaacagaaagaatatctacgccgaagaaaaacaggccaccgccgtggccacacgggttgtccacgacaacttcgccaaatttcacacccagtctaagattatcggcgtgatcaaaaacaagtaccccgagatcctgcagagcgtcgagatggaactgatggacgaacttgggggaatgaagatcaccgatatcttcagtatcaacagctacagcaagtggatgacccaggagggaatcgacttcatcaacaaaatcatcggcggctacagccctagcgtgggcgtcaaagtgagaggcctgaacgagttcatcaacctgtacagacagcagcacgaggaagccaacgccgaccggcggaacctggctaagatgcctatgctgtttaaacaaattctgagcgacatcagcacccggagcttcatccctgtgatgttcgagaatgacgccgagctcaaggacagcatcgaggccttcctgacaggcctgaatgatttcgagctgaacgctcagaagttcaacgttgtggtggccctggggaacctgtttcagaagattgtgccttgtgaaggcatcttcctggacgctgccctgatggaaaaggtttccaagacagctacaggcgactggagcctgctcgcacagtctatggaagcctacgccgaaacagcctttacaagagccaaggaccgggacgcctggctgagaaagaattactacagcctgtccgagctgagccaggtgccaatcctgaagaacactgatgagggcatgctgaagttcgagctgagcgcctactggtccggcgagaaaatggaatctttcgtgaagggcatcatggacgccgagctggccatgaagccagtgctggccagcatcggccagaaaaccgaagaggtgcggctgagagatagaatcgacgacgtggtgaagatcaagggctacctggacagcatccagaatttcctgcaccacctgaagcctttctgtgcccctaccgagctgaaccgggacgccgacttctactctgacttcgatgctctgtacaatcaactggtgctggtgattcccctgtacaactgcgtgagaaactacgtcacccaaaaggttaccgaggtgcagaagctgcgcctcaagttcgatgcacctaccctggccgatggatgggacgccaataaagagaatgacaacaaagccgtcctgttcgagaaagacggcctgtatttcctcggcatcctcaaccctaacctgaaagccaaggaccggcctgtgttcgaacatgaaagcaacgtgaccaagaagtcatgctaccggaagattgtgtacaaactgctgccaggccctaacaagatgctgcctaaggtgttctttgccgatagcaacaggacactgtaccaccctagcaagagcctgctggaccggtatcacaacggcgagtacaagaagggcgatagctttgatatcaagttttgccacgagctgatcgactacttcaaggcctctatctctattcaccctgactggaaggagttcggctttcaattttctgccacaaagacctacgagtctatcgacggcttctatagagaggtggaagagcagggctacaaggtgaacttcgcctttgtgcgtgctgacctgatcgataagtacgtggaaagcggctccctgttcctgttccagctctataacaaggacttcagctgtgcctctagcggcaagccgaatcttcatacactgtactggaaaagcctgttcgccaaggagaacctggacgagcctatactgaagctgtgcggcggcgccgagctgttcttcagacccgtggcgatccagaaaccctacgtgcacacattgggcgaaaagctggtgaatagacggctcggcgagcacggcaagggcgaggctatccctgagcgggtgcacaaggaactggtggactactacaaccacagagtgagcgtgctcagtcacgatggaaaggccttcaaggacaaggtggtggttcgggacgtggcccacagcatcaccaaggaccgacggtttagcgaggccaagttcttcttccacgtgcccatcatgtttaaccggaccgccagcaagagcgccaagttcaacgacaaggtggtggactacctgaaaaccacccaaaacgtgaacgtgatcggactggacagaggtgaaagaaacctgatctacctcacaatggtgaacctgcatggcaagctcatcgagcagcggagcttcaacctggtgaatggcgtggactaccattctaagctggatctgcgcgagaaggaacgtatggatgctagagtgaactgggagaatatcggcggcataaaggatctgaaaaccggctacctgagcgccgtggtgcacgagatcgccaaaatgatggtgacaaacaacgccatcgtggtgctggaagatctgaactttggattcaagagaggcagattcaaagtggaaaagcaggtgtaccagaaattcgagaagatgctgatcgacaaactgaacttcctgatgttcaaagagtgcaaccaggccgccctgggcggcgtgcggcgggcctatcagctgaccgacaagttcgtgagcttcgagaagctgggaaagcagaccggcttcctgttctatgtgcccgccggctatacaagcaaaatcgatcctacaaccggtttcaccaacctgttcaataccaagaaatgcaccaacgccgagggaagaaaggtgttcttcgaggctatgaacagcatcatctacgacggctccagaaaatctttcgcctttagcttcgactacggcaaccccgtgtttcgagcctcccagaccagcttcaagaaggaatggaccgtgtacagcgccgatacaagaatcgtgtataatcggggcgaaaagaccgtaaacaccatccaccctacccagatcctgcacgacgccctgtgcgccttgggaatcgacgtgcacgatgggttaaatgtcttgaacgtcgtgagagagacacccgctgataagatccacgccaagttcttcagcgatctcttctacgccttcgacagaaccctgcagatgaggaactctgtgagcgggaccgacgaagattacatccagagccctgtgctgaatgctaccggcgagttctttgacagcagaaaagccgacagcaccctgccccaggacgcagacgctaatggagcctaccacatcgccctgaagggcctgctgctcctgcagagaatgaaggatatcggctcagatatcaagctggatctgtctattaagcacgaggattggttcgccttcgctcagaagcggtgccagagatctagaaagcggacagcagacggctccgaatttgaaagccctaagaaaaagagaaaggtgggatccggcaaacctatccccaatcccctgctgggcctggacagcacctga

SEQ ID NO: 85 - protein, 1309 aa, unidentified
STINKFCGQGNGYSRSITLRNKLIPIGKTEENLKWFLEKDLERAIAYPEIKNLIDNIHRSVIEDTLSKVALNWNEIFNTLAAYQNEKDKKKKAAIKKDLEKLQGCARKKIVDTFKKNPDYEKLFKEGLFKELLPELIKTAPVSEIEDKTKALECFNRFSTYFTGFHENRKNMYSEDAKSTAISYRIVNENFPKFFANIKLYNYLKEKFPQIIINTEESLKDYLKGKKLDSVFSIDGFNDVLAQSGIDFYNTVIGGISGEAGTEKTQGLNEKINLARQQLPKDEKDKLRGKMVDLFKQILSDRETSSFIPTGFENKKEVYSTVKKFSEIVVEKSVSKVKEIFTQNEEYNLNEIFVPAKSLTNFSQNIFGNWSILSEGLFLLEKDNVKKQLSEKQIETLHKEIAKKDCSFTELQNAYERWCAENSVDATKNINRYFSIVDLRTKNDSFEKEEINILDEITNAFSKIDFDDIHDLQQEKEAATPIKNYLDEVQNLYHHLKLVDYRGEERKDANFYSKLDYILRKDRKDYLNLAEVVPLYNKVRNFVTKKPGEVKKIKMMFDCSSLLGGWGTDYETKEAHIFIDSGKYYLGIINEKLSKDDVELLKKSSERMITKVIYDFQKPDNKNTPRLFIRSKGTNYAPAVFQYNLPIESVIDIYDRGLFKTEYRKINSKVYKESLIKMIDYFKMGFERHESYKHYKFCWKESSKYNDIGEFYKDVINSCYQLNFEKVNYENLLKLVENNKLFLFQIYNKDFAEKKSGKKNLHTLYWENLFSEENLKDVCLKLNGEAELFWRKASLDKGKVIVHRMGSILVNRTTSEGKSIPEDIYQEIYQYKNKMKDKISDEAKSLLDSGTVICKEATHDITKDKRFTEDTYLFHCPITMNFKATDKKNKEFNNHVLEVLKENPDVKIIGLDRGERHLIYLSLINQKGEIELQKTLNLVEQVRNDKTVKVDYQEKLVHKEGDRDKARKNWQTIGNIKELKEGYLSAVVHEIAMLMVENNAIVVMEDLNFGFKRGRFAVERQIYQKFENMLIEKLNYLVFKDKNATEPGGVLNAYQLTNKSANVTDVYKQCGWLFYIPAAYTSKIDPKTGFANLFITKGLTNVEKKKEFFDKFDSIRYDSKEDCFVFGFDYAKLCDNASFRKKWEVYTRGERLVYNKDKHKNEPINPTEELKGIFDAFDINWNTDDNFIDSVQTIQAEKANAKFFDILLRMFNATLQMRNSKTNSSASEDDYLISPVKAEDGTFFDTREELKKGKDAKLPIDSDANGAYHIALKGLFLLENDFNRDEKGVIQNISNADWFKFVQEKKYKD

SEQ ID NO: 86 - protein, 1310 aa, unidentified
MSTINKFCGQGNGYSRSITLRNKLIPIGKTEENLKWFLEKDLERAIAYPEIKNLIDNIHRSVIEDTLSKVALNWNEIFNTLAAYQNEKDKKKKAAIKKDLEKLQGCARKKIVDTFKKNPDYEKLFKEGLFKELLPELIKTAPVSEIEDKTKALECFNRFSTYFTGFHENRKNMYSEDAKSTAISYRIVNENFPKFFANIKLYNYLKEKFPQIIINTEESLKDYLKGKKLDSVFSIDGFNDVLAQSGIDFYNTVIGGISGEAGTEKTQGLNEKINLARQQLPKDEKDKLRGKMVDLFKQILSDRETSSFIPTGFENKKEVYSTVKKFSEIVVEKSVSKVKEIFTQNEEYNLNEIFVPAKSLTNFSQNIFGNWSILSEGLFLLEKDNVKKQLSEKQIETLHKEIAKKDCSFTELQNAYERWCAENSVDATKNINRYFSIVDLRTKNDSFEKEEINILDEITNAFSKIDFDDIHDLQQEKEAATPIKNYLDEVQNLYHHLKLVDYRGEERKDANFYSKLDYILRKDRKDYLNLAEVVPLYNKVRNFVTKKPGEVKKIKMMFDCSSLLGGWGTDYETKEAHIFIDSGKYYLGIINEKLSKDDVELLKKSSERMITKVIYDFQKPDNKNTPRLFIRSKGTNYAPAVFQYNLPIESVIDIYDRGLFKTEYRKINSKVYKESLIKMIDYFKMGFERHESYKHYKFCWKESSKYNDIGEFYKDVINSCYQLNFEKVNYENLLKLVENNKLFLFQIYNKDFAEKKSGKKNLHTLYWENLFSEENLKDVCLKLNGEAELFWRKASLDKGKVIVHRMGSILVNRTTSEGKSIPEDIYQEIYQYKNKMKDKISDEAKSLLDSGTVICKEATHDITKDKRFTEDTYLFHCPITMNFKATDKKNKEFNNHVLEVLKENPDVKIIGLDRGERHLIYLSLINQKGEIELQKTLNLVEQVRNDKTVKVDYQEKLVHKEGDRDKARKNWQTIGNIKELKEGYLSAVVHEIAMLMVENNAIVVMEDLNFGFKRGRFAVERQIYQKFENMLIEKLNYLVFKDKNATEPGGVLNAYQLTNKSANVTDVYKQCGWLFYIPAAYTSKIDPKTGFANLFITKGLTNVEKKKEFFDKFDSIRYDSKEDCFVFGFDYAKLCDNASFRKKWEVYTRGERLVYNKDKHKNEPINPTEELKGIFDAFDINWNTDDNFIDSVQTIQAEKANAKFFDILLRMFNATLQMRNSKTNSSASEDDYLISPVKAEDGTFFDTREELKKGKDAKLPIDSDANGAYHIALKGLFLLENDFNRDEKGVIQNISNADWFKFVQEKKYKD

SEQ ID NO: 87 - protein, 1349 aa, synthetic construct
MGSGSTINKFCGQGNGYSRSITLRNKLIPIGKTEENLKWFLEKDLERAIAYPEIKNLIDNIHRSVIEDTLSKVALNWNEIFNTLAAYQNEKDKKKKAAIKKDLEKLQGCARKKIVDTFKKNPDYEKLFKEGLFKELLPELIKTAPVSEIEDKTKALECFNRFSTYFTGFHENRKNMYSEDAKSTAISYRIVNENFPKFFANIKLYNYLKEKFPQIIINTEESLKDYLKGKKLDSVFSIDGFNDVLAQSGIDFYNTVIGGISGEAGTEKTQGLNEKINLARQQLPKDEKDKLRGKMVDLFKQILSDRETSSFIPTGFENKKEVYSTVKKFSEIVVEKSVSKVKEIFTQNEEYNLNEIFVPAKSLTNFSQNIFGNWSILSEGLFLLEKDNVKKQLSEKQIETLHKEIAKKDCSFTELQNAYERWCAENSVDATKNINRYFSIVDLRTKNDSFEKEEINILDEITNAFSKIDFDDIHDLQQEKEAATPIKNYLDEVQNLYHHLKLVDYRGEERKDANFYSKLDYILRKDRKDYLNLAEVVPLYNKVRNFVTKKPGEVKKIKMMFDCSSLLGGWGTDYETKEAHIFIDSGKYYLGIINEKLSKDDVELLKKSSERMITKVIYDFQKPDNKNTPRLFIRSKGTNYAPAVFQYNLPIESVIDIYDRGLFKTEYRKINSKVYKESLIKMIDYFKMGFERHESYKHYKFCWKESSKYNDIGEFYKDVINSCYQLNFEKVNYENLLKLVENNKLFLFQIYNKDFAEKKSGKKNLHTLYWENLFSEENLKDVCLKLNGEAELFWRKASLDKGKVIVHRMGSILVNRTTSEGKSIPEDIYQEIYQYKNKMKDKISDEAKSLLDSGTVICKEATHDITKDKRFTEDTYLFHCPITMNFKATDKKNKEFNNHVLEVLKENPDVKIIGLDRGERHLIYLSLINQKGEIELQKTLNLVEQVRNDKTVKVDYQEKLVHKEGDRDKARKNWQTIGNIKELKEGYLSAVVHEIAMLMVENNAIVVMEDLNFGFKRGRFAVERQIYQKFENMLIEKLNYLVFKDKNATEPGGVLNAYQLTNKSANVTDVYKQCGWLFYIPAAYTSKIDPKTGFANLFITKGLTNVEKKKEFFDKFDSIRYDSKEDCFVFGFDYAKLCDNASFRKKWEVYTRGERLVYNKDKHKNEPINPTEELKGIFDAFDINWNTDDNFIDSVQTIQAEKANAKFFDILLRMFNATLQMRNSKTNSSASEDDYLISPVKAEDGTFFDTREELKKGKDAKLPIDSDANGAYHIALKGLFLLENDFNRDEKGVIQNISNADWFKFVQEKKYKDSRKRTADGSEFESPKKKRKVGSGKPIPNPLLGLDST

SEQ ID NO: 88 - DNA, 3933 bp, unidentified
  source 1..3933: mol_type = unassigned DNA
atgtcaactattaacaaattttgtggacaggggaatgggtattctcgttcaattactttgaggaataagttaattcctattggaaaaactgaagaaaatttgaaatggtttttagaaaaagatttggaaagggcaattgcttatccggagataaagaatcttatagataatattcatcgtagtgtaattgaggatactttatccaaagttgctttgaattggaatgaaatattcaatacacttgctgcttatcaaaatgaaaaagataaaaaaaagaaagcagcaataaaaaaggatttggagaaattacaaggttgtgcaagaaagaaaatagttgatacttttaaaaagaatcctgattatgaaaaattgtttaaggaaggattattcaaagaactattacctgagttaataaaaactgctcctgttagtgaaatagaagataaaacaaaagctttggaatgttttaatagatttagtacatattttacaggatttcatgaaaatagaaaaaatatgtatagcgaagatgcaaaatcaactgcaataagttaccgtattgtaaatgagaatttccccaaattttttgcaaatataaagttatataattatttaaaagaaaagtttccacaaattattattaatacagaagaatctttaaaagattatctaaaaggtaaaaaacttgattctgtatttagtattgatggatttaatgatgttttagctcaaagtggaatcgatttttataatacagtaattggtggaatttctggtgaagccggaacagaaaagactcaaggattaaatgaaaaaatcaatcttgcaagacaacaattaccaaaagatgaaaaagataaacttcgtggaaaaatggttgatttatttaagcagattttaagtgatagagaaacatcttcgtttattccaactggttttgaaaataaaaaagaagtttattctactgtaaagaaatttagtgaaattgttgttgaaaagtctgtttcaaaagtaaaagaaatttttacacaaaatgaagaatataatcttaatgaaatctttgttccagcaaaatcattaacaaatttttctcaaaatatttttggaaattggtctattttatcagaagggttatttttgcttgaaaaagataatgttaaaaaacaattatctgaaaaacaaattgaaacattacacaaagaaattgcaaaaaaagattgttcttttactgaactacaaaatgcttatgaaagatggtgtgctgaaaatagtgttgatgcaacaaaaaatatcaataggtatttttcaatagttgatttaagaacaaaaaatgattcgtttgaaaaagaagaaattaatattttggatgaaattacaaatgctttttcaaaaattgattttgatgatattcatgatttacaacaagaaaaagaagctgcaacaccaataaaaaattatttggatgaagttcaaaatctttatcatcacttaaaacttgttgattatcgtggtgaagaacgaaaggatgcaaacttttattcaaagctagattatatattaaggaaagataggaaagattaccttaatcttgctgaagttgtacctttgtataacaaagttcgtaattttgtaacaaagaaacctggtgaagtaaaaaagattaaaatgatgtttgattgtagttctttattaggggggtggggaactgattacgaaacaaaagaagctcatatttttattgattctggaaaatattatttgggaattataaacgaaaaattatcaaaagatgatgttgagttattaaaaaaatcaagtgaaagaatgataacaaaagtaatttatgattttcagaaacctgataataaaaatacacctcgtttatttattcgttcaaaaggaacaaattatgcacctgctgtttttcaatataatttaccaatagaatctgttattgatatttatgatagaggattgtttaaaaccgaatatagaaaaatcaattcaaaagtttacaaagaatcattaataaaaatgattgattatttcaagatgggctttgaaagacatgaatcatataagcattataaattctgttggaaggaatcttcaaaatataatgatattggtgaattttacaaggatgtgataaattcatgctatcaattaaatttcgaaaaagtgaattatgaaaatttattaaaattggttgaaaacaataaattattccttttccaaatatataacaaagattttgcagaaaaaaaatctggaaagaaaaatcttcatactttgtattgggaaaatctttttagtgaagaaaacttgaaagatgtttgcttaaaattgaatggtgaagctgaacttttctggcgcaaagcaagtttagacaaaggaaaagttatagttcatagaatgggttctattcttgtaaatagaactacatctgaaggtaaatcaattccagaagatatttatcaggaaatttatcaatataaaaataaaatgaaagataaaatttctgatgaagcaaaaagtcttttagattcaggaacagttatttgtaaagaagcaactcacgatattacaaaagacaagcgctttacagaagatacatatcttttccattgtccaattacaatgaactttaaagcaactgataaaaaaaataaagaatttaataatcatgttcttgaagttttaaaagaaaatccagatgttaaaattattggtcttgaccgtggtgaaagacatttgatttatctttctttgattaatcaaaaaggtgaaattgaacttcaaaaaacattgaatcttgtagaacaagttagaaatgataaaactgtaaaagtagattatcaagaaaaacttgtacataaagaaggcgacagagacaaagctcgtaaaaactggcaaacaattggaaatatcaaagaactaaaagaaggttatttatctgctgttgttcatgaaattgcaatgttgatggtagaaaataatgcaattgttgtaatggaagatttgaattttggatttaaacgtggtcgatttgctgtagaaagacaaatttatcaaaagtttgaaaatatgctcattgaaaaacttaattatcttgtgtttaaggataaaaatgctacagaaccaggtggtgtccttaatgcatatcaattaacaaataaatctgcaaatgtaactgacgtttataaacaatgtggatggcttttctatattccagcagcgtatacttcaaaaattgatccaaaaacaggttttgcaaatttattcataacaaaaggattaacaaatgtagaaaagaaaaaagaattctttgataaattcgattccattcgttatgactcaaaagaagactgttttgtatttggttttgattatgcaaaactttgtgataatgcaagttttagaaaaaaatgggaagtatacacaagaggggaaagattagtttacaataaagataaacataaaaatgaacctattaatccaacagaagaattaaaaggaatttttgatgcattcgatataaattggaatacggatgataattttattgattccgtacagacaatacaagcagaaaaagcaaatgccaaattctttgatattcttttgcgaatgtttaatgcaactcttcaaatgcgaaattcaaaaacaaattcttcagcatcagaagatgattatttgatatctccggtaaaagcagaggatggaacattctttgatactcgtgaagaattaaagaaaggcaaagatgcaaaacttcctatagattcagatgcaaacggagcttatcatattgcactaaaaggacttttcttacttgaaaatgacttcaatagagatgaaaaaggtgtgattcagaatatctccaacgccgattggtttaagtttgtgcaggagaaaaaatacaaagattaa

SEQ ID NO: 89 - DNA, 3927 bp, synthetic construct
  source 1..3927: mol_type = other DNA
agcaccatcaacaaattctgcggccagggcaacggctacagcagaagcatcaccctgcggaacaaactgatccctatcggcaagactgaggagaacctgaagtggttcctggagaaggacctggagcgggctatcgcctaccccgagattaaaaaccttatcgacaatatccacagaagcgtgatagaggataccctgagcaaggtcgccctgaactggaatgagatcttcaacaccctggccgcctaccagaacgagaaagataagaaaaagaaggccgctatcaagaaggacctggagaagttgcaaggatgtgcgagaaagaaaatcgtggataccttcaagaagaaccctgattatgagaaactgtttaaagagggactgttcaaggagctgctgcctgaactgatcaagaccgcccctgtgagcgaaattgaagataaaaccaaagccctggagtgcttcaaccggttctccacatacttcaccggcttccacgaaaatcgcaaaaatatgtacagcgaggacgcgaagagcaccgccatctcctaccggatcgtgaacgagaacttccccaagttcttcgctaatatcaagctgtacaactacctcaaggaaaaatttccacagattatcatcaacacagaagagtctctgaaggattacctgaagggcaagaagctggattccgtgttctccatcgacgggttcaatgacgtgctggcccagagcggcatagacttctacaacaccgtgatcggtggcatctcaggagaggccggcacagaaaagacccagggcctgaatgagaagatcaacctagccagacagcagctgcctaaggatgagaaggacaagctaagaggcaagatggtcgacctgttcaagcagattctgagcgatagagaaaccagcagcttcatccctactggcttcgagaataagaaggaagtgtactctaccgtgaagaagttcagcgaaatcgtggtcgaaaaaagcgtgtccaaggtgaaggagatcttcactcagaacgaagagtacaatctgaacgagatcttcgtgcctgcgaagagcctgaccaattttagccagaacatctttggcaactggagcatcctttctgaaggcctgttcctgctggaaaaggacaacgtgaagaaacagctgagtgagaaacaaatcgagacactccataaggagatcgccaagaaggactgcagctttaccgaactgcagaacgcctacgagcggtggtgcgccgagaactccgtggacgccaccaagaacattaacagatacttcagcatcgtcgacctgagaaccaagaatgactccttcgagaaggaagagatcaatatccttgatgagataaccaacgccttctctaagattgacttcgacgatatccacgatctgcagcaagagaaggaggccgccacccctatcaagaactacctggacgaggttcaaaacctgtaccaccacctgaagctggtggactacagaggtgaggaacgaaaggacgctaacttctactctaaactggactatatcctgagaaaggacagaaaggactacctgaacctggccgaagtggtgccattgtacaacaaggttagaaacttcgtgaccaagaagcctggcgaggtgaaaaagatcaagatgatgttcgactgcagcagcctgctgggcggatggggcacagattacgagacaaaagaggcccacattttcatcgactccggcaagtattaccttggaatcatcaacgagaagttgtcaaaagatgacgtggagctgctgaagaagagcagcgaacggatgatcacaaaggtgatctacgatttccaaaagcccgataacaagaatacacctagactgttcatcaggagcaagggcacaaattatgctcctgctgttttccaatacaatctgccaatagagtctgtgatcgatatttacgaccgtggcctgtttaagaccgagtacagaaaaatcaacagcaaggtgtacaaggagagcctgattaagatgatcgattacttcaagatgggctttgagagacacgagagctacaagcactacaagttttgctggaaggaatctagcaagtacaacgacatcggcgaattttacaaggatgtgattaactcttgttaccagctgaacttcgagaaggtgaactatgagaacctcctgaagttagtggaaaacaacaagctgttcctgtttcagatctacaacaaggattttgccgaaaagaaaagcggtaagaagaacctgcacaccctgtactgggagaacctgttttctgaggagaacctgaaggacgtttgtctgaagctgaatggcgaggccgagctgttctggcggaaggcttctctggacaagggcaaggtgatcgtgcacagaatgggctctatcctggtgaacagaacaacaagcgagggcaagtcaatccctgaggacatctaccaggagatctatcagtacaagaacaaaatgaaggataagatcagcgacgaagccaaaagcctgctggacagcggcaccgtgatctgtaaagaagccacccacgacatcaccaaggacaaacggttcacagaggacacctacctgttccactgccctatcaccatgaacttcaaggccaccgacaagaaaaacaaagagttcaacaaccacgtgctggaagtgctgaaagagaatcccgacgtgaagatcatcggcctggacagaggcgaacggcacctgatctacctgagcctgatcaaccagaagggcgagatcgagctgcagaaaaccctgaatctggtggaacaggtgcggaacgacaaaaccgtgaaggtggactaccaggagaagctggtgcataaggaaggcgaccgcgacaaagccagaaagaactggcagacaatcggaaacatcaaggaactgaaggagggctacctgtctgccgtggtgcacgaaatcgccatgctgatggtggaaaacaacgccatcgtggtgatggaggacctgaacttcggcttcaagagaggcagattcgccgtggaacggcagatctaccagaagttcgagaacatgctgatcgaaaagctgaactacctagtgttcaaggacaagaacgccaccgaacctggcggcgtgctgaatgcgtatcagctcaccaacaagagcgccaacgtcaccgacgtgtacaaacagtgcggctggctgttctacatccccgccgcttatacaagcaagatcgaccccaagaccggattcgccaacctgttcatcacaaagggactgacaaacgtggaaaagaagaaggagttcttcgataagttcgacagcatccggtacgacagcaaagaggactgctttgtgttcggcttcgactacgccaagctgtgcgacaacgcctcctttagaaagaagtgggaagtttacaccagaggagagaggctggtctacaacaaagacaagcacaaaaacgaacctatcaaccccaccgaggagctgaagggcatcttcgatgcttttgatattaactggaacaccgacgacaacttcattgattcagtgcagaccatccaggccgagaaggccaacgccaagttctttgacatcctgctgagaatgttcaacgccacactgcagatgagaaacagcaagactaactcctctgccagcgaggacgactacctgatcagccctgtcaaagccgaggatggcaccttcttcgacacaagagaggaattaaagaagggcaaagatgccaagctgccgatcgacagcgacgctaatggcgcctaccacatcgccctgaaaggactgttcctgctggaaaatgactttaaccgggacgagaagggagtgatccaaaatatcagcaacgctgattggttcaagtttgtgcaggagaagaaatacaaggat

SEQ ID NO: 90 - DNA, 4050 bp, synthetic construct
  source 1..4050: mol_type = other DNA
atgggctccggaagcaccatcaacaaattctgcggccagggcaacggctacagcagaagcatcaccctgcggaacaaactgatccctatcggcaagactgaggagaacctgaagtggttcctggagaaggacctggagcgggctatcgcctaccccgagattaaaaaccttatcgacaatatccacagaagcgtgatagaggataccctgagcaaggtcgccctgaactggaatgagatcttcaacaccctggccgcctaccagaacgagaaagataagaaaaagaaggccgctatcaagaaggacctggagaagttgcaaggatgtgcgagaaagaaaatcgtggataccttcaagaagaaccctgattatgagaaactgtttaaagagggactgttcaaggagctgctgcctgaactgatcaagaccgcccctgtgagcgaaattgaagataaaaccaaagccctggagtgcttcaaccggttctccacatacttcaccggcttccacgaaaatcgcaaaaatatgtacagcgaggacgcgaagagcaccgccatctcctaccggatcgtgaacgagaacttccccaagttcttcgctaatatcaagctgtacaactacctcaaggaaaaatttccacagattatcatcaacacagaagagtctctgaaggattacctgaagggcaagaagctggattccgtgttctccatcgacgggttcaatgacgtgctggcccagagcggcatagacttctacaacaccgtgatcggtggcatctcaggagaggccggcacagaaaagacccagggcctgaatgagaagatcaacctagccagacagcagctgcctaaggatgagaaggacaagctaagaggcaagatggtcgacctgttcaagcagattctgagcgatagagaaaccagcagcttcatccctactggcttcgagaataagaaggaagtgtactctaccgtgaagaagttcagcgaaatcgtggtcgaaaaaagcgtgtccaaggtgaaggagatcttcactcagaacgaagagtacaatctgaacgagatcttcgtgcctgcgaagagcctgaccaattttagccagaacatctttggcaactggagcatcctttctgaaggcctgttcctgctggaaaaggacaacgtgaagaaacagctgagtgagaaacaaatcgagacactccataaggagatcgccaagaaggactgcagctttaccgaactgcagaacgcctacgagcggtggtgcgccgagaactccgtggacgccaccaagaacattaacagatacttcagcatcgtcgacctgagaaccaagaatgactccttcgagaaggaagagatcaatatccttgatgagataaccaacgccttctctaagattgacttcgacgatatccacgatctgcagcaagagaaggaggccgccacccctatcaagaactacctggacgaggttcaaaacctgtaccaccacctgaagctggtggactacagaggtgaggaacgaaaggacgctaacttctactctaaactggactatatcctgagaaaggacagaaaggactacctgaacctggccgaagtggtgccattgtacaacaaggttagaaacttcgtgaccaagaagcctggcgaggtgaaaaagatcaagatgatgttcgactgcagcagcctgctgggcggatggggcacagattacgagacaaaagaggcccacattttcatcgactccggcaagtattaccttggaatcatcaacgagaagttgtcaaaagatgacgtggagctgctgaagaagagcagcgaacggatgatcacaaaggtgatctacgatttccaaaagcccgataacaagaatacacctagactgttcatcaggagcaagggcacaaattatgctcctgctgttttccaatacaatctgccaatagagtctgtgatcgatatttacgaccgtggcctgtttaagaccgagtacagaaaaatcaacagcaaggtgtacaaggagagcctgattaagatgatcgattacttcaagatgggctttgagagacacgagagctacaagcactacaagttttgctggaaggaatctagcaagtacaacgacatcggcgaattttacaaggatgtgattaactcttgttaccagctgaacttcgagaaggtgaactatgagaacctcctgaagttagtggaaaacaacaagctgttcctgtttcagatctacaacaaggattttgccgaaaagaaaagcggtaagaagaacctgcacaccctgtactgggagaacctgttttctgaggagaacctgaaggacgtttgtctgaagctgaatggcgaggccgagctgttctggcggaaggcttctctggacaagggcaaggtgatcgtgcacagaatgggctctatcctggtgaacagaacaacaagcgagggcaagtcaatccctgaggacatctaccaggagatctatcagtacaagaacaaaatgaaggataagatcagcgacgaagccaaaagcctgctggacagcggcaccgtgatctgtaaagaagccacccacgacatcaccaaggacaaacggttcacagaggacacctacctgttccactgccctatcaccatgaacttcaaggccaccgacaagaaaaacaaagagttcaacaaccacgtgctggaagtgctgaaagagaatcccgacgtgaagatcatcggcctggacagaggcgaacggcacctgatctacctgagcctgatcaaccagaagggcgagatcgagctgcagaaaaccctgaatctggtggaacaggtgcggaacgacaaaaccgtgaaggtggactaccaggagaagctggtgcataaggaaggcgaccgcgacaaagccagaaagaactggcagacaatcggaaacatcaaggaactgaaggagggctacctgtctgccgtggtgcacgaaatcgccatgctgatggtggaaaacaacgccatcgtggtgatggaggacctgaacttcggcttcaagagaggcagattcgccgtggaacggcagatctaccagaagttcgagaacatgctgatcgaaaagctgaactacctagtgttcaaggacaagaacgccaccgaacctggcggcgtgctgaatgcgtatcagctcaccaacaagagcgccaacgtcaccgacgtgtacaaacagtgcggctggctgttctacatccccgccgcttatacaagcaagatcgaccccaagaccggattcgccaacctgttcatcacaaagggactgacaaacgtggaaaagaagaaggagttcttcgataagttcgacagcatccggtacgacagcaaagaggactgctttgtgttcggcttcgactacgccaagctgtgcgacaacgcctcctttagaaagaagtgggaagtttacaccagaggagagaggctggtctacaacaaagacaagcacaaaaacgaacctatcaaccccaccgaggagctgaagggcatcttcgatgcttttgatattaactggaacaccgacgacaacttcattgattcagtgcagaccatccaggccgagaaggccaacgccaagttctttgacatcctgctgagaatgttcaacgccacactgcagatgagaaacagcaagactaactcctctgccagcgaggacgactacctgatcagccctgtcaaagccgaggatggcaccttcttcgacacaagagaggaattaaagaagggcaaagatgccaagctgccgatcgacagcgacgctaatggcgcctaccacatcgccctgaaaggactgttcctgctggaaaatgactttaaccgggacgagaagggagtgatccaaaatatcagcaacgctgattggttcaagtttgtgcaggagaagaaatacaaggattctagaaagcggacagcagacggctccgaatttgaaagccctaagaaaaagagaaaggtgggatccggcaaacctatccccaatcccctgctgggcctggacagcacctga

SEQ ID NO: 91 - protein, 1282 aa, unidentified
GKFFETDEFIGQYSINKTLRFELIPQGKTKELLNNYMNDNSKIKQDILRADEKNNFKEVIDEYYRELIHDALTDEDIFSITPLVKDAYELYIASRKNTSDSSKKEYRDVKNKIRKEIANILNKYKTIYGLDKFANIYKSESDKSVDDDESDNDDLDEKNTTNDDNAKSEDKRIYKWLNKKLRLKQISNEEYDRYYKSLNEYHGFTTGLQGLQNNKENMFSSENKSTAIAFRIIDDNMEKYFSNILLLEFIKNKYKDLYEKIEEKANKMNVECFTKYFTQEGIDEYNQMIGRSIEEEYAKGINQEINLYKQSKGLNNKEIRTLSPLYKQILSKTSQNEIIVFKNDKETLEYIKNICDYIIEEDIFGKMNHLIKTNLIDMCTGIYIKRNELSNISFKLYNDWGLLDRIICDYANEFKTKKEKNEFEKLNKEVISLNLLNDIFNKYKETRGNDTDLKEIVEYFKNVDEKMIEDEYSKIKSILNLERIDIDRRVPSKDEEKGGEGFEQICMIKTFLDLLLESIHIYKPLSLIKNGEKVEIYNYNENFYNEYDILFSQLDNIINLYNKVRNYFSKKTYSKEKIKIYFSKPTLLNGWDVNKEISNYSIILRKDEEYFLAIMNSDNKIFTNERLEENCAITENNEECYEKMVYKQISDSNKMFSKVFFSEKNKKIYMPSEEIKNIRKNKTHLKVANNKDSQTKWIKFMIECYYKHPEWSKYFDINFKKPEEYESIVEFYNQVNEKIYNIKFVNIKCDYINSMVDSGELYLFKIYNKDFSKNKKKSGTDNLHTMYWKLLFSKENMNCGVYKLNGQAEVFFRKASLPDKITHERNKEIDNKNPIKDKKTSTFTYDLKKDKRFMEDKFFFHCPITINYKGLNAKDKEIRKYNEKINKFIAGNPDINIIGIDRGERHLLYYTIINQKGEILKQSTLNNVGIEGRDKDYQELLSNKEKERHLARKSWGTIGNIKELKEGYLSIVVHELAKLVKEYNAIIVLENLNAGFKRGRTKVEKQVYQKFELALIKKLNYLVFKNENIQNKGGYLKGLQLTQPFDTFKDIGNQSGIIYYVIPSYTSKICPTTGFIDVIKPQYESVEKAKELFSKFKRIYFDNNKKCFIFEFMYKDFGRDYGLDKIWSICTLGEKRYYYDSKNKVSNVINVTESIISILQEKNINYINSDNIIDEILQYSDVKLYKELLFNLKVVLQMRYTKSGTNNEDDFILSPVLDENDKAFCSLNAKETEPQNADANGAYHIAMKGLNAIMSIKNGNVDRDINNLENWINFIQKFHIGK

SEQ ID NO: 92 - protein, 1283 aa, unidentified
MGKFFETDEFIGQYSINKTLRFELIPQGKTKELLNNYMNDNSKIKQDILRADEKNNFKEVIDEYYRELIHDALTDEDIFSITPLVKDAYELYIASRKNTSDSSKKEYRDVKNKIRKEIANILNKYKTIYGLDKFANIYKSESDKSVDDDESDNDDLDEKNTTNDDNAKSEDKRIYKWLNKKLRLKQISNEEYDRYYKSLNEYHGFTTGLQGLQNNKENMFSSENKSTAIAFRIIDDNMEKYFSNILLLEFIKNKYKDLYEKIEEKANKMNVECFTKYFTQEGIDEYNQMIGRSIEEEYAKGINQEINLYKQSKGLNNKEIRTLSPLYKQILSKTSQNEIIVFKNDKETLEYIKNICDYIIEEDIFGKMNHLIKTNLIDMCTGIYIKRNELSNISFKLYNDWGLLDRIICDYANEFKTKKEKNEFEKLNKEVISLNLLNDIFNKYKETRGNDTDLKEIVEYFKNVDEKMIEDEYSKIKSILNLERIDIDRRVPSKDEEKGGEGFEQICMIKTFLDLLLESIHIYKPLSLIKNGEKVEIYNYNENFYNEYDILFSQLDNIINLYNKVRNYFSKKTYSKEKIKIYFSKPTLLNGWDVNKEISNYSIILRKDEEYFLAIMNSDNKIFTNERLEENCAITENNEECYEKMVYKQISDSNKMFSKVFFSEKNKKIYMPSEEIKNIRKNKTHLKVANNKDSQTKWIKFMIECYYKHPEWSKYFDINFKKPEEYESIVEFYNQVNEKIYNIKFVNIKCDYINSMVDSGELYLFKIYNKDFSKNKKKSGTDNLHTMYWKLLFSKENMNCGVYKLNGQAEVFFRKASLPDKITHERNKEIDNKNPIKDKKTSTFTYDLKKDKRFMEDKFFFHCPITINYKGLNAKDKEIRKYNEKINKFIAGNPDINIIGIDRGERHLLYYTIINQKGEILKQSTLNNVGIEGRDKDYQELLSNKEKERHLARKSWGTIGNIKELKEGYLSIVVHELAKLVKEYNAIIVLENLNAGFKRGRTKVEKQVYQKFELALIKKLNYLVFKNENIQNKGGYLKGLQLTQPFDTFKDIGNQSGIIYYVIPSYTSKICPTTGFIDVIKPQYESVEKAKELFSKFKRIYFDNNKKCFIFEFMYKDFGRDYGLDKIWSICTLGEKRYYYDSKNKVSNVINVTESIISILQEKNINYINSDNIIDEILQYSDVKLYKELLFNLKVVLQMRYTKSGTNNEDDFILSPVLDENDKAFCSLNAKETEPQNADANGAYHIAMKGLNAIMSIKNGNVDRDINNLENWINFIQKFHIGK

SEQ ID NO: 93 - protein, 1322 aa, synthetic construct
MGSGGKFFETDEFIGQYSINKTLRFELIPQGKTKELLNNYMNDNSKIKQDILRADEKNNFKEVIDEYYRELIHDALTDEDIFSITPLVKDAYELYIASRKNTSDSSKKEYRDVKNKIRKEIANILNKYKTIYGLDKFANIYKSESDKSVDDDESDNDDLDEKNTTNDDNAKSEDKRIYKWLNKKLRLKQISNEEYDRYYKSLNEYHGFTTGLQGLQNNKENMFSSENKSTAIAFRIIDDNMEKYFSNILLLEFIKNKYKDLYEKIEEKANKMNVECFTKYFTQEGIDEYNQMIGRSIEEEYAKGINQEINLYKQSKGLNNKEIRTLSPLYKQILSKTSQNEIIVFKNDKETLEYIKNICDYIIEEDIFGKMNHLIKTNLIDMCTGIYIKRNELSNISFKLYNDWGLLDRIICDYANEFKTKKEKNEFEKLNKEVISLNLLNDIFNKYKETRGNDTDLKEIVEYFKNVDEKMIEDEYSKIKSILNLERIDIDRRVPSKDEEKGGEGFEQICMIKTFLDLLLESIHIYKPLSLIKNGEKVEIYNYNENFYNEYDILFSQLDNIINLYNKVRNYFSKKTYSKEKIKIYFSKPTLLNGWDVNKEISNYSIILRKDEEYFLAIMNSDNKIFTNERLEENCAITENNEECYEKMVYKQISDSNKMFSKVFFSEKNKKIYMPSEEIKNIRKNKTHLKVANNKDSQTKWIKFMIECYYKHPEWSKYFDINFKKPEEYESIVEFYNQVNEKIYNIKFVNIKCDYINSMVDSGELYLFKIYNKDFSKNKKKSGTDNLHTMYWKLLFSKENMNCGVYKLNGQAEVFFRKASLPDKITHERNKEIDNKNPIKDKKTSTFTYDLKKDKRFMEDKFFFHCPITINYKGLNAKDKEIRKYNEKINKFIAGNPDINIIGIDRGERHLLYYTIINQKGEILKQSTLNNVGIEGRDKDYQELLSNKEKERHLARKSWGTIGNIKELKEGYLSIVVHELAKLVKEYNAIIVLENLNAGFKRGRTKVEKQVYQKFELALIKKLNYLVFKNENIQNKGGYLKGLQLTQPFDTFKDIGNQSGIIYYVIPSYTSKICPTTGFIDVIKPQYESVEKAKELFSKFKRIYFDNNKKCFIFEFMYKDFGRDYGLDKIWSICTLGEKRYYYDSKNKVSNVINVTESIISILQEKNINYINSDNIIDEILQYSDVKLYKELLFNLKVVLQMRYTKSGTNNEDDFILSPVLDENDKAFCSLNAKETEPQNADANGAYHIAMKGLNAIMSIKNGNVDRDINNLENWINFIQKFHIGKSRKRTADGSEFESPKKKRKVGSGKPIPNPLLGLDST

SEQ ID NO: 94 - DNA, 3852 bp, unidentified
  source 1..3852: mol_type = unassigned DNA
atgggtaaattttttgaaacagatgaatttattggacagtattcaataaataaaacattacgattcgaattgataccacaaggtaagacaaaggaattactaaataattatatgaatgataacagcaaaattaaacaggatattttaagagcagatgaaaagaataattttaaagaagtaattgatgaatattatcgagagttgattcatgatgctttaacagatgaagatattttttccattacaccattagtaaaggatgcatatgaattatatattgcttctagaaaaaatacttctgatagttctaaaaaagaatatagagatgttaaaaataaaattaggaaagaaatagcaaacattcttaataaatataagacgatttatggactagataaatttgcaaatatatataaatccgagagtgataaaagtgtagatgatgatgaatctgataatgatgatttagatgagaaaaatactactaatgatgataatgcaaaatcagaagataaaaggatatacaaatggctaaataaaaaattaagattaaaacaaatttctaacgaggaatatgatagatactacaaatctttaaatgaatatcatggttttacaacaggtctgcaaggattacaaaataataaagaaaatatgttctcttcagaaaacaaaagtacggcaatagcatttcgaataatagatgacaatatggaaaaatatttttcaaatatactgttattagaatttattaaaaacaaatataaagatttatatgaaaaaattgaagaaaaagcaaataaaatgaatgtggaatgttttactaaatattttacacaagagggtatagatgaatataatcaaatgataggtagaagtatagaagaagaatatgcaaaaggtataaatcaagaaataaatctttataaacaatcaaaaggattaaataataaagaaattaggacattatctccattgtataagcaaatattatcaaagacttcacaaaatgaaataatagtattcaaaaatgataaagaaactttagaatacatcaagaatatatgtgattatataatagaagaagatatatttggaaagatgaatcatttaattaaaacaaatttgattgatatgtgtactggtatatatataaaaagaaatgaattatcgaatatttcatttaaactttataatgattggggattactagatagaataatatgtgattatgcaaatgaatttaagacaaaaaaagaaaagaacgaatttgaaaaattaaataaagaagtaatttcacttaatcttttaaatgatatatttaataaatataaggaaacaagagggaatgatacagatttaaaagaaatagtagaatattttaaaaatgtagatgaaaaaatgatagaggatgaatactctaaaataaaaagtattttaaatttagaaagaatagatattgatagaagagtaccaagcaaagatgaagaaaaaggtggagaaggatttgaacaaatttgtatgataaaaacatttttagatttattgcttgagagtatacatatttacaaaccattaagtttaattaaaaatggagagaaagtggagatatataattataatgaaaatttttacaatgaatatgatatattgttttcacaattagataatataattaacttatataataaagtcagaaattatttttctaaaaaaacatattcaaaagaaaaaatcaagatatatttttctaagccaacgttattaaatggatgggatgtaaataaagaaatatcaaattattcgattattttgagaaaagatgaagaatatttcctagccataatgaatagtgataataagatttttactaatgaaagattggaagaaaattgcgcaattacagaaaataatgaagagtgttatgaaaaaatggtatataaacaaatatccgattcaaataagatgttttcaaaagtgtttttttcagaaaaaaacaaaaaaatatatatgccttcagaagaaattaaaaatattagaaaaaataaaacacatttgaaagtagcaaataataaagactcacaaacaaaatggattaaatttatgattgaatgctattataaacatcctgaatggagtaaatattttgatataaattttaaaaagcctgaagaatatgaatcaatagttgaattttataatcaagtaaatgaaaaaatatataatataaaatttgtaaatattaaatgtgattatataaatagtatggttgatagtggagaattgtatttgtttaaaatatataataaggatttttcaaaaaataagaaaaaatctggaacagataatttacacactatgtattggaaattattattttcaaaagaaaatatgaattgtggtgtatacaaattaaatggacaagcagaagtgttttttaggaaagcttctttacctgataaaattacacatgaaagaaataaagaaatagataataaaaatccaataaaagataaaaaaacaagtacatttacttatgatttaaagaaagataaaagattcatggaagataaattcttctttcattgcccaataacaataaattataaaggattaaatgcaaaagataaagaaataagaaaatataatgagaaaataaacaaatttattgctggtaacccagatataaatattatcggaatagatcgtggtgaacgacatttgctatattatacgataataaatcaaaagggtgaaatattaaaacagtcaacattaaataatgttggtattgaagggcgtgataaagattatcaagaattattatctaataaagagaaagaacgtcacttagctagaaaaagttggggaacaataggtaatataaaagaacttaaagaaggatatttatcaattgtagtacatgaattagctaaattagtaaaggaatataatgcaataattgttctagaaaatttgaatgctggatttaaaaggggaagaactaaagttgaaaaacaagtatatcaaaaatttgaacttgcattgataaagaaacttaattatttagtatttaaaaacgaaaatattcaaaataaaggtggttatttaaaaggattacaattaactcagccatttgatacttttaaagatattggaaatcaatctggtataatttattatgttattccatcatatacatcgaaaatatgtcctactacaggctttatagatgtaattaagccacaatatgaaagtgttgaaaaagccaaagaattattttctaaatttaagcgtatatatttcgataataataaaaaatgttttatatttgaatttatgtataaagactttggtagagattatggtttagataaaatatggagtatatgtacacttggagaaaaaagatattattatgattctaaaaataaagtatcaaatgtaataaatgtaacagaatcaataattagtatattacaagaaaaaaacataaattatataaattcagacaatatcatagatgaaattttacaatatagtgatgttaagttgtataaagaattattatttaatttaaaagttgttttacaaatgagatatacgaagagtggtacaaataatgaagatgattttattctatcaccagtattagatgaaaatgataaggcattttgttcacttaatgcaaaagaaacagaacctcaaaatgcagatgcaaacggtgcatatcatattgctatgaaaggtttaaatgcaataatgagcattaagaatggtaatgtagatagagatattaacaatttagaaaattggataaattttatacaaaagtttcatataggtaaataa

SEQ ID NO: 95 - DNA, 3846 bp, synthetic construct
  source 1..3846: mol_type = other DNA
ggcaagttctttgaaaccgacgagttcatcggacagtacagtatcaacaaaacactgaggttcgagctcatccctcaaggcaagaccaaggaactgctgaacaactatatgaacgacaacagtaagatcaagcaggacatcctgcgggccgacgagaagaacaatttcaaggaagtgatcgacgagtattatagagagttgatccacgacgccctgaccgacgaggacatcttttccatcacccctctcgtcaaggacgcctacgagctgtacatcgcctccagaaaaaacaccagcgactccagcaagaaggagtatcgggacgtgaaaaataagattagaaaagagatcgctaacatcctgaacaagtacaagacaatctacggcctggacaagttcgccaatatctacaagtctgagagcgacaagagcgttgatgatgacgaatctgataacgatgacttggacgagaagaataccaccaacgacgataatgccaagtctgaggacaagcggatctataagtggctgaataagaagctgagactgaagcagatctccaacgaagaatacgaccggtactacaagtccctgaacgaataccacgggttcacaacaggactgcagggcctgcagaacaacaaggaaaacatgttcagcagcgagaacaagagcaccgccatcgcctttagaatcatcgatgacaacatggaaaagtatttttctaacatcctgctcctggagttcatcaaaaacaagtacaaagatctgtacgagaagatcgaggagaaggccaacaagatgaacgtggaatgcttcaccaagtacttcacccaggagggcatcgacgagtacaatcagatgattggcagaagcattgaggaagaatacgccaagggcatcaaccaggagatcaacctgtataagcagagcaagggtctaaacaataaggagatcagaacactgagccccctgtacaagcagatcctgtccaagaccagccagaacgaaatcatcgtgttcaaaaacgacaaggaaaccctggaatacatcaagaatatctgtgattacattatcgaggaggacatcttcggaaagatgaaccacctgatcaaaaccaacctgatcgacatgtgcaccggaatctacattaagagaaacgagctgagcaacatctctttcaagctctacaacgactggggcctgctggacagaattatctgtgactacgccaacgagttcaagacaaagaaggaaaagaatgagttcgagaagctgaacaaagaggtgatctctctgaacctgctcaacgatattttcaacaaatacaaggaaaccagaggcaatgatacagacctgaaggaaatcgtggaatactttaaaaacgtcgacgagaaaatgattgaggacgagtacagcaagatcaagagcatacttaatctggaacgcatcgacatcgaccgtagagtgccaagcaaggacgaggaaaagggcggcgaaggctttgagcagatctgcatgatcaagacgttcctggatctgctgttggagagcatccacatctacaagcctctgtctctgatcaagaacggcgagaaggtggaaatctacaattataacgagaacttctacaacgagtacgacatcctgttcagccagctggataacattataaatctgtacaataaggtgcggaactacttcagcaagaaaacctacagcaaagagaaaatcaaaatctatttctccaaacccaccctgctgaacggatgggacgtgaacaaggagatcagcaactactctatcatcctgagaaaagacgaagagtactttctggcaattatgaacagcgacaacaagatcttcacgaatgagaggctggaagaaaactgcgccatcaccgagaataatgaagaatgttacgagaaaatggtgtacaagcaaatctctgactctaacaagatgttcagcaaggtgtttttcagcgagaaaaacaagaagatctacatgcccagcgaagagatcaagaatatcagaaagaacaagacccatctcaaggtggccaacaataaggattctcaaacaaagtggatcaagttcatgatcgagtgctactataaacaccctgagtggagtaagtacttcgatatcaacttcaagaaacctgaagaatatgaaagcatcgtggaattttacaaccaggtgaacgagaagatctacaacatcaagttcgtgaatatcaaatgcgactacatcaacagcatggtggattcgggagagctgtacctgttcaagatctacaacaaggacttctctaagaacaagaaaaaaagtggcacagataacctgcacaccatgtattggaagctgctgtttagcaaagaaaacatgaattgcggcgtgtacaagctgaacggccaggccgaggtgttcttcagaaaggccagcctgcctgataagatcacacacgaaagaaataaggagatcgacaacaaaaatcctatcaaggacaagaaaaccagcaccttcacatacgacctgaagaaagataagcggttcatggaagataagttcttcttccactgccccataaccatcaactacaagggccttaacgccaaggacaaggagatcagaaagtacaacgaaaagatcaacaaattcatcgctggcaaccccgacatcaacatcataggcatcgaccggggcgaacggcacctgctgtactacaccatcatcaaccagaagggagagatcctgaagcaatctacactgaacaacgtgggcatcgagggcagagacaaagattaccaggagctgctgagcaacaaggaaaaggaaagacacctcgctagaaagagctggggcaccatcggcaacataaaagaactgaaggaaggctacctgagcatcgtggtgcacgagctggccaagctcgtgaaggagtacaacgccatcatcgtgctggagaatctgaacgccggcttcaagagaggcagaaccaaggtggaaaaacaggtctaccagaagtttgagctggccctgatcaagaagctgaactacctcgtgttcaagaacgagaacatccagaacaagggaggctacctgaagggactgcaactgacacagcctttcgacacctttaaggatatcggcaaccagagcggcatcatctactacgtgatccccagctacacaagcaaaatttgtccaacaaccggcttcatcgacgtgatcaaacctcagtacgagtctgtggaaaaggccaaggagctgttctccaaattcaaacggatttacttcgacaacaacaagaagtgctttatcttcgaatttatgtacaaagatttcggcagagattacggtctggacaagatctggagcatctgtaccctgggcgagaagagatactactacgacagcaagaacaaggtttccaatgtgatcaacgtgaccgagagcatcatcagcatcctgcaggagaagaacatcaactacatcaacagcgacaacatcatcgacgagatcctgcagtacagcgacgtgaagctgtataaggagctgctttttaacctgaaggtggtgctgcagatgcggtacaccaagagcggcaccaataacgaggacgacttcattctgtctcctgtgctggacgagaacgacaaggccttctgcagcctgaacgctaaggaaacagagcctcagaatgctgatgctaatggcgcctatcatatcgccatgaagggactgaacgccatcatgtccatcaagaacggcaacgtggatagagatattaacaacctggaaaactggatcaacttcatccagaaattccacatcgggaag

SEQ ID NO: 96 - DNA, 3969 bp, synthetic construct
  source 1..3969: mol_type = other DNA
atgggctccggaggcaagttctttgaaaccgacgagttcatcggacagtacagtatcaacaaaacactgaggttcgagctcatccctcaaggcaagaccaaggaactgctgaacaactatatgaacgacaacagtaagatcaagcaggacatcctgcgggccgacgagaagaacaatttcaaggaagtgatcgacgagtattatagagagttgatccacgacgccctgaccgacgaggacatcttttccatcacccctctcgtcaaggacgcctacgagctgtacatcgcctccagaaaaaacaccagcgactccagcaagaaggagtatcgggacgtgaaaaataagattagaaaagagatcgctaacatcctgaacaagtacaagacaatctacggcctggacaagttcgccaatatctacaagtctgagagcgacaagagcgttgatgatgacgaatctgataacgatgacttggacgagaagaataccaccaacgacgataatgccaagtctgaggacaagcggatctataagtggctgaataagaagctgagactgaagcagatctccaacgaagaatacgaccggtactacaagtccctgaacgaataccacgggttcacaacaggactgcagggcctgcagaacaacaaggaaaacatgttcagcagcgagaacaagagcaccgccatcgcctttagaatcatcgatgacaacatggaaaagtatttttctaacatcctgctcctggagttcatcaaaaacaagtacaaagatctgtacgagaagatcgaggagaaggccaacaagatgaacgtggaatgcttcaccaagtacttcacccaggagggcatcgacgagtacaatcagatgattggcagaagcattgaggaagaatacgccaagggcatcaaccaggagatcaacctgtataagcagagcaagggtctaaacaataaggagatcagaacactgagccccctgtacaagcagatcctgtccaagaccagccagaacgaaatcatcgtgttcaaaaacgacaaggaaaccctggaatacatcaagaatatctgtgattacattatcgaggaggacatcttcggaaagatgaaccacctgatcaaaaccaacctgatcgacatgtgcaccggaatctacattaagagaaacgagctgagcaacatctctttcaagctctacaacgactggggcctgctggacagaattatctgtgactacgccaacgagttcaagacaaagaaggaaaagaatgagttcgagaagctgaacaaagaggtgatctctctgaacctgctcaacgatattttcaacaaatacaaggaaaccagaggcaatgatacagacctgaaggaaatcgtggaatactttaaaaacgtcgacgagaaaatgattgaggacgagtacagcaagatcaagagcatacttaatctggaacgcatcgacatcgaccgtagagtgccaagcaaggacgaggaaaagggcggcgaaggctttgagcagatctgcatgatcaagacgttcctggatctgctgttggagagcatccacatctacaagcctctgtctctgatcaagaacggcgagaaggtggaaatctacaattataacgagaacttctacaacgagtacgacatcctgttcagccagctggataacattataaatctgtacaataaggtgcggaactacttcagcaagaaaacctacagcaaagagaaaatcaaaatctatttctccaaacccaccctgctgaacggatgggacgtgaacaaggagatcagcaactactctatcatcctgagaaaagacgaagagtactttctggcaattatgaacagcgacaacaagatcttcacgaatgagaggctggaagaaaactgcgccatcaccgagaataatgaagaatgttacgagaaaatggtgtacaagcaaatctctgactctaacaagatgttcagcaaggtgtttttcagcgagaaaaacaagaagatctacatgcccagcgaagagatcaagaatatcagaaagaacaagacccatctcaaggtggccaacaataaggattctcaaacaaagtggatcaagttcatgatcgagtgctactataaacaccctgagtggagtaagtacttcgatatcaacttcaagaaacctgaagaatatgaaagcatcgtggaattttacaaccaggtgaacgagaagatctacaacatcaagttcgtgaatatcaaatgcgactacatcaacagcatggtggattcgggagagctgtacctgttcaagatctacaacaaggacttctctaagaacaagaaaaaaagtggcacagataacctgcacaccatgtattggaagctgctgtttagcaaagaaaacatgaattgcggcgtgtacaagctgaacggccaggccgaggtgttcttcagaaaggccagcctgcctgataagatcacacacgaaagaaataaggagatcgacaacaaaaatcctatcaaggacaagaaaaccagcaccttcacatacgacctgaagaaagataagcggttcatggaagataagttcttcttccactgccccataaccatcaactacaagggccttaacgccaaggacaaggagatcagaaagtacaacgaaaagatcaacaaattcatcgctggcaaccccgacatcaacatcataggcatcgaccggggcgaacggcacctgctgtactacaccatcatcaaccagaagggagagatcctgaagcaatctacactgaacaacgtgggcatcgagggcagagacaaagattaccaggagctgctgagcaacaaggaaaaggaaagacacctcgctagaaagagctggggcaccatcggcaacataaaagaactgaaggaaggctacctgagcatcgtggtgcacgagctggccaagctcgtgaaggagtacaacgccatcatcgtgctggagaatctgaacgccggcttcaagagaggcagaaccaaggtggaaaaacaggtctaccagaagtttgagctggccctgatcaagaagctgaactacctcgtgttcaagaacgagaacatccagaacaagggaggctacctgaagggactgcaactgacacagcctttcgacacctttaaggatatcggcaaccagagcggcatcatctactacgtgatccccagctacacaagcaaaatttgtccaacaaccggcttcatcgacgtgatcaaacctcagtacgagtctgtggaaaaggccaaggagctgttctccaaattcaaacggatttacttcgacaacaacaagaagtgctttatcttcgaatttatgtacaaagatttcggcagagattacggtctggacaagatctggagcatctgtaccctgggcgagaagagatactactacgacagcaagaacaaggtttccaatgtgatcaacgtgaccgagagcatcatcagcatcctgcaggagaagaacatcaactacatcaacagcgacaacatcatcgacgagatcctgcagtacagcgacgtgaagctgtataaggagctgctttttaacctgaaggtggtgctgcagatgcggtacaccaagagcggcaccaataacgaggacgacttcattctgtctcctgtgctggacgagaacgacaaggccttctgcagcctgaacgctaaggaaacagagcctcagaatgctgatgctaatggcgcctatcatatcgccatgaagggactgaacgccatcatgtccatcaagaacggcaacgtggatagagatattaacaacctggaaaactggatcaacttcatccagaaattccacatcgggaagtctagaaagcggacagcagacggctccgaatttgaaagccctaagaaaaagagaaaggtgggatccggcaaacctatccccaatcccctgctgggcctggacagcacctga

SEQ ID NO: 97 - protein, 1234 aa, unidentified
EFDNSFVNRYPLSKTLSFSLLPVGSTEANFEKKLLLQEDEKRAAEYILVKSYIDRYHKAYIESVLSKVVLDGINNYAQLYCKNNKTEQDIKRLEQLEGSFRKQISKSLKSDARYKLIYKKEMLEKLLPEFLDNEEEKARVISFENFTTYFTGFHTNRENMYTDEAKSTAVSFRCINDNLPKFLDNISVFKWVTAFLSESDINELKADFSGLLGCSLEEMFTPDYFSFVLSQSGIERYNNVIGGYTCSDGEKVKGLNEYINLYNQKLQHGEKKLPLLKRLFKQILSDTESVSFIPEKLENDDAVISAINGFCNIKIENETFFEILDKTKCLFSNLNEFDSAGVYITNGFAVTDISNAVFGTWDVISEAWKKEYAKAIPLKNIAKADAYYEKQGKAYKAIKSFSVSELQRLANTTEGKAAYKHNGDISAYFSETVCFAVQDIFEKYSSSKALFASPYKNEKRLFKNNEAIALIKDFLDSIKNLEKLIKPFNGSGRENDKDESFYGEFTACYERLSKIDLLYDKVRNYMTQKPYSGDKIKLNFENPQFLNGWDRNKERDYRTVLLRKGGYYYLAIMDKSNNRIFEDLPEPKNGEDCYEKIDYKLLPGPNKMLPKVFFAASNIDYFAPSEQILKIRQKETFKKGVNFNIDDCHAFIDFLKESIEKHDEWCKYGFEFKDTSDYNNIGEFYKDVREQGYSISFRNVPESYINSCVNSGSLYLFQIYNKDFSPYSKGTKSLHTLYFEMLFDERNLKNVVYQLNGGAEMFYRKASIKERDKIVHPANIPIKNKNPDNPKAESVFEYDIIKDRRFTERQFSLHIPVTLNFKGSGGSANLNADVRRAIRGADENYVIGIDRGERNLLYITVINSKGEIVEQIPGNVIINGKQVVDYHKLLDAKEKERLAARQNWTTVENIKELKEGYLSVIIHNICELVKKYNAVIAMEDLSSGFKNSRVKVEKQVYQKFEKMLTEKLNFLVDKKADVQSRGGLLQAYQLTNSTKDYKRAGSQDGIVFYVPAWLTSKIDPVTGFVDLLKPKYTSVQEAKELFSNFEAVEYIPEEDLFSFTFDYSKFPRCSVAYRNKWTVYSNGERIYTFRDKNSNNEYVSKTVALTTEFKSLFDEYSVYYRDNLKSQILCQDKVDFFKQLIRLLSLTMQMRNSISNSAVDYLISPVKDKNGNFFDSRKSIKNLPENADANGAYNIAKKALWAIGQIKEADENDLMKVKLSVSNKEWLKYVQEVE

SEQ ID NO: 98 - protein, 1235 aa, unidentified
MEFDNSFVNRYPLSKTLSFSLLPVGSTEANFEKKLLLQEDEKRAAEYILVKSYIDRYHKAYIESVLSKVVLDGINNYAQLYCKNNKTEQDIKRLEQLEGSFRKQISKSLKSDARYKLIYKKEMLEKLLPEFLDNEEEKARVISFENFTTYFTGFHTNRENMYTDEAKSTAVSFRCINDNLPKFLDNISVFKWVTAFLSESDINELKADFSGLLGCSLEEMFTPDYFSFVLSQSGIERYNNVIGGYTCSDGEKVKGLNEYINLYNQKLQHGEKKLPLLKRLFKQILSDTESVSFIPEKLENDDAVISAINGFCNIKIENETFFEILDKTKCLFSNLNEFDSAGVYITNGFAVTDISNAVFGTWDVISEAWKKEYAKAIPLKNIAKADAYYEKQGKAYKAIKSFSVSELQRLANTTEGKAAYKHNGDISAYFSETVCFAVQDIFEKYSSSKALFASPYKNEKRLFKNNEAIALIKDFLDSIKNLEKLIKPFNGSGRENDKDESFYGEFTACYERLSKIDLLYDKVRNYMTQKPYSGDKIKLNFENPQFLNGWDRNKERDYRTVLLRKGGYYYLAIMDKSNNRIFEDLPEPKNGEDCYEKIDYKLLPGPNKMLPKVFFAASNIDYFAPSEQILKIRQKETFKKGVNFNIDDCHAFIDFLKESIEKHDEWCKYGFEFKDTSDYNNIGEFYKDVREQGYSISFRNVPESYINSCVNSGSLYLFQIYNKDFSPYSKGTKSLHTLYFEMLFDERNLKNVVYQLNGGAEMFYRKASIKERDKIVHPANIPIKNKNPDNPKAESVFEYDIIKDRRFTERQFSLHIPVTLNFKGSGGSANLNADVRRAIRGADENYVIGIDRGERNLLYITVINSKGEIVEQIPGNVIINGKQVVDYHKLLDAKEKERLAARQNWTTVENIKELKEGYLSVIIHNICELVKKYNAVIAMEDLSSGFKNSRVKVEKQVYQKFEKMLTEKLNFLVDKKADVQSRGGLLQAYQLTNSTKDYKRAGSQDGIVFYVPAWLTSKIDPVTGFVDLLKPKYTSVQEAKELFSNFEAVEYIPEEDLFSFTFDYSKFPRCSVAYRNKWTVYSNGERIYTFRDKNSNNEYVSKTVALTTEFKSLFDEYSVYYRDNLKSQILCQDKVDFFKQLIRLLSLTMQMRNSISNSAVDYLISPVKDKNGNFFDSRKSIKNLPENADANGAYNIAKKALWAIGQIKEADENDLMKVKLSVSNKEWLKYVQEVE

SEQ ID NO: 99 - protein, 1274 aa, synthetic construct
MGSGEFDNSFVNRYPLSKTLSFSLLPVGSTEANFEKKLLLQEDEKRAAEYILVKSYIDRYHKAYIESVLSKVVLDGINNYAQLYCKNNKTEQDIKRLEQLEGSFRKQISKSLKSDARYKLIYKKEMLEKLLPEFLDNEEEKARVISFENFTTYFTGFHTNRENMYTDEAKSTAVSFRCINDNLPKFLDNISVFKWVTAFLSESDINELKADFSGLLGCSLEEMFTPDYFSFVLSQSGIERYNNVIGGYTCSDGEKVKGLNEYINLYNQKLQHGEKKLPLLKRLFKQILSDTESVSFIPEKLENDDAVISAINGFCNIKIENETFFEILDKTKCLFSNLNEFDSAGVYITNGFAVTDISNAVFGTWDVISEAWKKEYAKAIPLKNIAKADAYYEKQGKAYKAIKSFSVSELQRLANTTEGKAAYKHNGDISAYFSETVCFAVQDIFEKYSSSKALFASPYKNEKRLFKNNEAIALIKDFLDSIKNLEKLIKPFNGSGRENDKDESFYGEFTACYERLSKIDLLYDKVRNYMTQKPYSGDKIKLNFENPQFLNGWDRNKERDYRTVLLRKGGYYYLAIMDKSNNRIFEDLPEPKNGEDCYEKIDYKLLPGPNKMLPKVFFAASNIDYFAPSEQILKIRQKETFKKGVNFNIDDCHAFIDFLKESIEKHDEWCKYGFEFKDTSDYNNIGEFYKDVREQGYSISFRNVPESYINSCVNSGSLYLFQIYNKDFSPYSKGTKSLHTLYFEMLFDERNLKNVVYQLNGGAEMFYRKASIKERDKIVHPANIPIKNKNPDNPKAESVFEYDIIKDRRFTERQFSLHIPVTLNFKGSGGSANLNADVRRAIRGADENYVIGIDRGERNLLYITVINSKGEIVEQIPGNVIINGKQVVDYHKLLDAKEKERLAARQNWTTVENIKELKEGYLSVIIHNICELVKKYNAVIAMEDLSSGFKNSRVKVEKQVYQKFEKMLTEKLNFLVDKKADVQSRGGLLQAYQLTNSTKDYKRAGSQDGIVFYVPAWLTSKIDPVTGFVDLLKPKYTSVQEAKELFSNFEAVEYIPEEDLFSFTFDYSKFPRCSVAYRNKWTVYSNGERIYTFRDKNSNNEYVSKTVALTTEFKSLFDEYSVYYRDNLKSQILCQDKVDFFKQLIRLLSLTMQMRNSISNSAVDYLISPVKDKNGNFFDSRKSIKNLPENADANGAYNIAKKALWAIGQIKEADENDLMKVKLSVSNKEWLKYVQEVESRKRTADGSEFESPKKKRKVGSGKPIPNPLLGLDST

SEQ ID NO: 100 - DNA, 3708 bp, unidentified
  source 1..3708: mol_type = unassigned DNA
atggaattcgacaatagctttgttaaccgataccctttatcaaaaacactaagcttcagtttgcttcctgttggcagtaccgaagcaaattttgagaaaaaactgttgctgcaggaggacgaaaaaagagccgcggaatatattttggtgaagtcatacattgacagataccataaagcctatattgaatcggttttatcaaaggttgtgcttgacggcataaataactatgcacagctgtactgcaagaacaacaaaaccgaacaggatatcaaacgactggagcagcttgaaggttcatttagaaagcagatttcaaagagcttgaaatccgatgcccgttataagttgatttataaaaaagaaatgcttgaaaagcttttgcctgagtttcttgataatgaagaagaaaaggcgagggtaatatcttttgaaaactttacaacatatttcacaggctttcataccaatagagaaaatatgtataccgacgaagcaaaatccactgcggtgtccttcagatgtataaatgataatttaccaaaatttcttgataatatttcagtttttaaatgggttacggcatttttgagcgaaagtgatatcaacgaattaaaggcggatttttcaggtctgttaggttgttcgcttgaagaaatgtttacaccggattatttttcctttgtgttatctcaaagcgggatagaaagatataacaatgttatcggcggttacacatgttctgacggtgaaaaagttaagggactaaatgaatacataaatttatacaaccaaaagttacaacacggtgaaaaaaagctcccgcttttaaaacgcttgttcaagcagatattgagtgataccgaaagtgtatcctttattccggaaaagcttgaaaacgacgatgctgttatttctgcgataaacggattttgtaatatcaaaattgaaaacgaaacattctttgaaattcttgataaaactaaatgcttgttttcaaatttaaatgagtttgacagcgccggtgtatatattaccaacggttttgctgtaaccgatatttcaaatgctgttttcggtacttgggatgttatttcggaagcgtggaaaaaggagtatgcgaaagcaatcccgcttaaaaatatcgccaaggcagatgcatattacgaaaagcagggcaaggcgtataaggcaattaaaagcttttcggtaagcgagcttcaaaggctggccaacacaacagaagggaaggcggcatataagcacaacggagatatttctgcatatttttcggaaactgtttgctttgcggttcaagatatatttgaaaaatacagtagttcaaaagccctttttgcgtcgccctataaaaatgaaaagcggctcttcaaaaacaatgaggctatagcgctgattaaggattttcttgacagcatcaaaaatctggaaaagcttattaaaccatttaacggctccggtagagaaaacgataaggacgaaagcttctacggtgaatttaccgcttgctacgagaggctttctaaaattgacctgctatatgataaggttcgcaactatatgacacaaaaaccttattccggggacaagataaagttgaattttgaaaatccgcaatttctaaatggttgggacaggaacaaagagcgggattacagaactgttctcttaagaaaaggcgggtattactaccttgctattatggataaaagcaacaacaggatttttgaagatttgccggagcccaaaaacggcgaggattgttatgaaaaaatagactacaagcttctgccgggaccgaataagatgttgccaaaggtgttttttgccgcgagcaatattgattattttgcaccctctgagcaaattttgaaaattagacagaaagaaacctttaagaagggtgtgaattttaatattgatgattgccatgctttcatagacttccttaaagagtctatagaaaaacacgatgagtggtgcaagtatgggttcgaatttaaagatacttcagattataacaacatcggtgaattttataaagatgtaagggagcagggctattctatcagctttagaaatgtgcctgagtcttatataaattcttgcgttaattccggttcactttaccttttccaaatctacaacaaggatttttcaccttacagcaaagggaccaagagtttgcacacattgtattttgaaatgctttttgatgaaaggaaccttaagaatgttgtttatcagcttaacggcggtgcagagatgttttaccgcaaagcaagtattaaggaaagggataaaatagtacaccctgctaatattccgataaaaaataaaaatcccgataacccaaaagctgaaagtgtttttgagtatgacatcataaaggacagacgctttactgaaagacagttctctttgcatattcctgttacgctcaattttaaaggctcgggcggctctgcaaatcttaatgctgatgtgcgcagagccataagaggcgctgatgaaaactatgttataggtatagacagaggagaaagaaatttgctttacatcaccgtaataaacagtaaaggtgaaattgttgagcagattccgggcaatgtaataatcaacggaaaacaagtggtcgattatcacaagctgcttgatgccaaagaaaaagagcgtcttgcagcacggcaaaactggacaacggttgaaaatatcaaggagcttaaagagggctatttgagcgtaatcatacacaatatttgtgaacttgtaaaaaaatacaatgctgttattgctatggaggatctttcttccggttttaaaaacagcagggttaaagtagaaaaacaggtttatcagaaatttgaaaaaatgcttaccgaaaagcttaattttcttgttgataaaaaagctgatgttcaaagcaggggaggacttctgcaggcatatcagttaacaaacagcaccaaggattataagcgggcaggctcacaagacggtattgttttctatgttccggcgtggcttacaagcaaaatcgatcccgttacgggttttgttgatttgcttaagcctaagtatacaagtgtgcaggaagcaaaggagctgttttcaaattttgaagctgttgaatatatccctgaggaggatttgttcagctttacttttgattattcgaaatttccccgttgctccgtagcttaccgtaacaaatggactgtatactcaaacggcgaaagaatttatacattcagggataaaaacagcaataatgaatatgttagcaaaacagttgctcttacaacggagtttaaatccttgtttgacgaatacagcgtttattaccgcgataaccttaaatcgcagattctatgtcaagataaagtcgatttcttcaaacagctaattcggttactgtctttgacaatgcaaatgcgaaacagtatttcaaattcagcagtagattatctgatttctccggttaaggataaaaacggaaatttctttgacagccggaaaagtataaaaaatcttccggaaaatgcagatgctaacggtgcttacaacattgccaaaaaggctctttgggcaatcgggcaaataaaggaagcggatgagaatgatttaatgaaggtcaagctgtctgtttcaaacaaggaatggcttaaatatgtgcaggaggtagaatga

SEQ ID NO: 101 - DNA, 3702 bp, synthetic construct
  source 1..3702: mol_type = other DNA
gaatttgataactctttcgtgaatagatatcctctgagcaagaccctgagcttcagtctgctgccagtgggcagcaccgaagccaacttcgaaaaaaagctgctgctgcaggaggacgaaaagagagccgccgagtacatcctggtgaaaagctacatcgacagataccacaaggcctacatcgagagcgtgctgagcaaggtggtgctggacggcatcaacaactatgcccagctgtactgcaagaacaacaagaccgaacaggacatcaagcggctggagcagctggagggcagcttcagaaagcagatctctaaaagcctgaagtccgacgccagatacaagctgatctacaaaaaggagatgctggaaaagctcctgcctgagttcctggacaacgaggaagaaaaggctagagtgatcagcttcgagaactttacaacctacttcactggcttccacaccaaccgggaaaacatgtacaccgatgaggccaagtctacggccgtttcctttaggtgtatcaacgataacctgccaaagttcctggacaacatcagcgtattcaagtgggtcaccgcctttctgagcgagtctgacatcaacgaactgaaggccgatttcagcggcctgttgggctgctccctggaagagatgttcacccctgattacttcagcttcgtgctgtctcagagcggcatcgagagatacaacaacgtgatcggcggatacacctgtagcgatggcgagaaagtcaaaggacttaatgagtacatcaacctgtataaccagaagctgcaacacggcgaaaagaaactgcccctgctcaagcggctgttcaagcagattctgtcagacaccgagagcgtgtccttcatccccgagaaactggaaaatgatgacgccgtgatctccgccattaacggattttgtaatatcaagatcgagaatgaaacattcttcgagatcctggacaagaccaagtgcctgttcagcaatctgaacgagttcgactctgccggagtgtacatcaccaacggcttcgcagtgacagacatcagcaacgccgtgttcggcacctgggacgtcatcagcgaagcctggaagaaagagtacgccaaagctatccccctgaagaacatcgctaaggccgacgcctactatgagaagcagggcaaggcctacaaggccatcaagagcttctctgtaagcgaactgcagagactggccaacaccacggagggaaaggccgcctacaagcacaacggcgacatcagcgcctatttcagcgagacagtctgcttcgctgtgcaggatatcttcgagaagtatagcagcagcaaggccctgttcgccagcccctataagaacgagaagcggctgttcaagaacaatgaggcaatcgctctgattaaggacttcctggatagcatcaagaacctggagaagctgattaagccattcaacggcagcggcagagagaacgacaaggacgagagcttttacggcgagttcaccgcctgctacgagcggctgagcaaaatcgatctgctgtacgacaaggtgcggaactacatgacacagaaaccttacagcggcgataagatcaagctgaacttcgagaatcctcagttcctgaacggatgggatagaaacaaggagcgggattacagaacagtgctgctgagaaagggaggttattactacctggccatcatggacaagagcaacaaccggatcttcgaggatctgcctgagcctaagaatggtgaggactgctacgaaaaaatcgattacaagctgctgcctggccctaacaagatgctgcccaaagtgttcttcgccgctagtaacatcgactacttcgcccctagcgaacagatcctcaaaatccggcagaaggaaaccttcaaaaagggcgtgaacttcaacattgacgactgtcacgccttcatcgacttcctgaaggaatctatcgagaagcacgacgagtggtgcaagtacggcttcgagtttaaggacaccagcgactacaacaatataggcgagttctacaaggacgtgcgggaacagggctacagcatctcttttcggaatgtgcccgagtcctacatcaacagctgcgtgaactctggctctctgtacctgtttcagatctacaacaaagattttagcccttactctaagggcacaaagagcctgcacaccctgtactttgaaatgctgtttgacgagcgcaacctgaagaacgtggtgtatcagctgaatggtggcgctgagatgttctacagaaaggccagcatcaaggaaagagacaagatcgtgcaccccgccaacatccctatcaagaacaagaaccccgacaaccctaaggccgagagcgtgttcgaatacgacattatcaaggacagaagattcaccgaacggcagttctccctgcacatccctgtgaccctgaacttcaaaggctctggcggatctgccaacctgaacgccgacgttaggcgggctatcagaggcgccgatgagaactacgtgatcggcatcgaccggggcgagaggaacctgctgtacatcacagtgatcaatagcaagggcgagatcgtggaacaaatcccaggcaacgtgatcatcaacggcaagcaagtggtggactaccacaagctgctggatgctaaagagaaggaaagactggctgccagacagaactggacaacagttgaaaacatcaaggaactgaaggaaggctacctgtccgtgatcatccacaacatctgcgagctggtgaaaaagtacaacgctgtgatcgctatggaggacctgagcagcggcttcaagaacagccgcgtgaaggtggaaaagcaggtataccagaagttcgaaaaaatgctgaccgagaaactgaacttcctggtggacaagaaagccgatgtgcaaagcagaggcggcctgctgcaggcctaccagctgacaaatagcacaaaggattacaagcgggccggcagccaagacggcatcgtgttctacgtgcctgcctggctgacaagcaaaattgaccctgtgaccggctttgtggacctgctgaaacctaaatacaccagcgttcaggaggccaaagagctgttcagcaacttcgaggccgtcgagtacatccccgaggaggacctgttcagcttcaccttcgactacagcaagttccccagatgcagcgtggcctacagaaacaagtggaccgtgtacagtaacggagagagaatctacacattcagagataagaacagcaacaacgaatacgtgtccaagacagttgccctgaccaccgagtttaaaagcctcttcgacgaatatagcgtgtactaccgagacaacctgaagagtcagattttgtgccaggataaggtggatttcttcaagcaacttatcagactgctgtccctgaccatgcagatgagaaacagcatcagcaacagcgccgtggactacctgatctcccctgtgaaggataagaatggcaattttttcgacagcagaaagagcatcaagaacctgcctgagaacgccgacgccaacggcgcctacaacattgctaagaaggctctgtgggccatcggtcagatcaaagaggctgatgagaatgacctgatgaaggtgaagctgtccgtgtctaataaagagtggctgaagtacgtgcaggaggtggaa

SEQ ID NO: 102 - DNA, 3825 bp, synthetic construct
  source 1..3825: mol_type = other DNA
atgggctccggagaatttgataactctttcgtgaatagatatcctctgagcaagaccctgagcttcagtctgctgccagtgggcagcaccgaagccaacttcgaaaaaaagctgctgctgcaggaggacgaaaagagagccgccgagtacatcctggtgaaaagctacatcgacagataccacaaggcctacatcgagagcgtgctgagcaaggtggtgctggacggcatcaacaactatgcccagctgtactgcaagaacaacaagaccgaacaggacatcaagcggctggagcagctggagggcagcttcagaaagcagatctctaaaagcctgaagtccgacgccagatacaagctgatctacaaaaaggagatgctggaaaagctcctgcctgagttcctggacaacgaggaagaaaaggctagagtgatcagcttcgagaactttacaacctacttcactggcttccacaccaaccgggaaaacatgtacaccgatgaggccaagtctacggccgtttcctttaggtgtatcaacgataacctgccaaagttcctggacaacatcagcgtattcaagtgggtcaccgcctttctgagcgagtctgacatcaacgaactgaaggccgatttcagcggcctgttgggctgctccctggaagagatgttcacccctgattacttcagcttcgtgctgtctcagagcggcatcgagagatacaacaacgtgatcggcggatacacctgtagcgatggcgagaaagtcaaaggacttaatgagtacatcaacctgtataaccagaagctgcaacacggcgaaaagaaactgcccctgctcaagcggctgttcaagcagattctgtcagacaccgagagcgtgtccttcatccccgagaaactggaaaatgatgacgccgtgatctccgccattaacggattttgtaatatcaagatcgagaatgaaacattcttcgagatcctggacaagaccaagtgcctgttcagcaatctgaacgagttcgactctgccggagtgtacatcaccaacggcttcgcagtgacagacatcagcaacgccgtgttcggcacctgggacgtcatcagcgaagcctggaagaaagagtacgccaaagctatccccctgaagaacatcgctaaggccgacgcctactatgagaagcagggcaaggcctacaaggccatcaagagcttctctgtaagcgaactgcagagactggccaacaccacggagggaaaggccgcctacaagcacaacggcgacatcagcgcctatttcagcgagacagtctgcttcgctgtgcaggatatcttcgagaagtatagcagcagcaaggccctgttcgccagcccctataagaacgagaagcggctgttcaagaacaatgaggcaatcgctctgattaaggacttcctggatagcatcaagaacctggagaagctgattaagccattcaacggcagcggcagagagaacgacaaggacgagagcttttacggcgagttcaccgcctgctacgagcggctgagcaaaatcgatctgctgtacgacaaggtgcggaactacatgacacagaaaccttacagcggcgataagatcaagctgaacttcgagaatcctcagttcctgaacggatgggatagaaacaaggagcgggattacagaacagtgctgctgagaaagggaggttattactacctggccatcatggacaagagcaacaaccggatcttcgaggatctgcctgagcctaagaatggtgaggactgctacgaaaaaatcgattacaagctgctgcctggccctaacaagatgctgcccaaagtgttcttcgccgctagtaacatcgactacttcgcccctagcgaacagatcctcaaaatccggcagaaggaaaccttcaaaaagggcgtgaacttcaacattgacgactgtcacgccttcatcgacttcctgaaggaatctatcgagaagcacgacgagtggtgcaagtacggcttcgagtttaaggacaccagcgactacaacaatataggcgagttctacaaggacgtgcgggaacagggctacagcatctcttttcggaatgtgcccgagtcctacatcaacagctgcgtgaactctggctctctgtacctgtttcagatctacaacaaagattttagcccttactctaagggcacaaagagcctgcacaccctgtactttgaaatgctgtttgacgagcgcaacctgaagaacgtggtgtatcagctgaatggtggcgctgagatgttctacagaaaggccagcatcaaggaaagagacaagatcgtgcaccccgccaacatccctatcaagaacaagaaccccgacaaccctaaggccgagagcgtgttcgaatacgacattatcaaggacagaagattcaccgaacggcagttctccctgcacatccctgtgaccctgaacttcaaaggctctggcggatctgccaacctgaacgccgacgttaggcgggctatcagaggcgccgatgagaactacgtgatcggcatcgaccggggcgagaggaacctgctgtacatcacagtgatcaatagcaagggcgagatcgtggaacaaatcccaggcaacgtgatcatcaacggcaagcaagtggtggactaccacaagctgctggatgctaaagagaaggaaagactggctgccagacagaactggacaacagttgaaaacatcaaggaactgaaggaaggctacctgtccgtgatcatccacaacatctgcgagctggtgaaaaagtacaacgctgtgatcgctatggaggacctgagcagcggcttcaagaacagccgcgtgaaggtggaaaagcaggtataccagaagttcgaaaaaatgctgaccgagaaactgaacttcctggtggacaagaaagccgatgtgcaaagcagaggcggcctgctgcaggcctaccagctgacaaatagcacaaaggattacaagcgggccggcagccaagacggcatcgtgttctacgtgcctgcctggctgacaagcaaaattgaccctgtgaccggctttgtggacctgctgaaacctaaatacaccagcgttcaggaggccaaagagctgttcagcaacttcgaggccgtcgagtacatccccgaggaggacctgttcagcttcaccttcgactacagcaagttccccagatgcagcgtggcctacagaaacaagtggaccgtgtacagtaacggagagagaatctacacattcagagataagaacagcaacaacgaatacgtgtccaagacagttgccctgaccaccgagtttaaaagcctcttcgacgaatatagcgtgtactaccgagacaacctgaagagtcagattttgtgccaggataaggtggatttcttcaagcaacttatcagactgctgtccctgaccatgcagatgagaaacagcatcagcaacagcgccgtggactacctgatctcccctgtgaaggataagaatggcaattttttcgacagcagaaagagcatcaagaacctgcctgagaacgccgacgccaacggcgcctacaacattgctaagaaggctctgtgggccatcggtcagatcaaagaggctgatgagaatgacctgatgaaggtgaagctgtccgtgtctaataaagagtggctgaagtacgtgcaggaggtggaatctagaaagcggacagcagacggctccgaatttgaaagccctaagaaaaagagaaaggtgggatccggcaaacctatccccaatcccctgctgggcctggacagcacctga

SEQ ID NO: 103 - protein, 1242 aa, unidentified
KAETNLTELVNLYSLQKTLRFELIPQGKTLENIEKNGILTQDNQRADDYEKVKKLIDEYHKHHIEISLDDCRLEGLEEYKELYEKKDDLKKIQENLRKQIVKSLTENERYKDKRLFSDKLFKEDLPNYLKDREQDKALVKKFEKFTTYFTGFNENRKNMYSSEDKPTSIAYRLIHENLPKFIDNLHIFDKIKETTIKDDFDKIVEKLNKHLKIHIKSFDEIFSIEYFNKTLSQKQIDNYNNIIGGMSFENGTKIQGLNEYINRYNQKQEDKHQKLPCVKTLYKQILSDREKISWIPEQFDDDKQMAESISNLYNEMLPIIKDDLLPLMANIGDYDLSKIFISNDSALTTISQRIFGAYNVYTLAIIEKLKSDKPKSKRQSESKYLDEIDKNFKNMKSFSIAKLNNAVKGKYDKTIENYIKVFGAFDEEENLLQRLETAYNEAEPILNNIEDRCKNINQDKDAVEKIKTLLDALKDIQHFAKLLLCDNDETEIDAEFYNKLHDIWVKLDKITPIYNMVRNYVTKKPYSEEKIKLNFEKSTLLGGWDLNKEKNNLSVILRKDNLYYLGIMKKDNNKIFDSTNIKTDGVCFEKMEYKLLPDPKKMLPKVFFSKKCSKDFNPNDKILEIKENESFKKTSSNFNIEQCRKLIDFYKESINKHKDWQKFNFQFSDTKTYNDINEFYNEVEKQGYKISFCKISEDYINELVKDNKLYLFKIWNKDFSKYSKGTPNTHTLYWKQIFAPENINNVVYKLNGQAEIFFRQASISQKNVIKHLANKPVKNKNIKNEKKESTFSYDLVKDKRFTMDKFHFHVPITINFKAKGINNTNPIVNNLIRQNKIEHIIGIDRGERHLLYLSLIDLKGNIIEQKSLNEIINNYNGNEYKTDYHTLLDDKEKERKDARLSWNTIENIKELKDGYMSQVVHIISQMIVKYNAIVVLEDLNHGFVRGRQKIEKQVYEKFEHKLIDKLNYYVDKNADSNAVGGLYNALQLTNPFDSFEKLGKQSGCLFYIPAWKTSKIDPVTGFINMFTNLKYESVEKSKKFFSKFDDIRYNKEKNRFEFDVSFDKFDSDFVRITQESKLHWTLCSVGQRIELVKENNGYKPNEINLTDAFKSVFNTNKIEINTAKLNREIGKINDTAFFKELMRLMKLLLQMRNSKPNSIEKNDDYIISPVADENGVFFDSSKVEDNGNLPKDADANGAYNIARKGLYVIHQIKQSEDDKKIDFKDFNPRWLKFIQQKLYLND

SEQ ID NO: 104 - protein, 1243 aa, unidentified
MKAETNLTELVNLYSLQKTLRFELIPQGKTLENIEKNGILTQDNQRADDYEKVKKLIDEYHKHHIEISLDDCRLEGLEEYKELYEKKDDLKKIQENLRKQIVKSLTENERYKDKRLFSDKLFKEDLPNYLKDREQDKALVKKFEKFTTYFTGFNENRKNMYSSEDKPTSIAYRLIHENLPKFIDNLHIFDKIKETTIKDDFDKIVEKLNKHLKIHIKSFDEIFSIEYFNKTLSQKQIDNYNNIIGGMSFENGTKIQGLNEYINRYNQKQEDKHQKLPCVKTLYKQILSDREKISWIPEQFDDDKQMAESISNLYNEMLPIIKDDLLPLMANIGDYDLSKIFISNDSALTTISQRIFGAYNVYTLAIIEKLKSDKPKSKRQSESKYLDEIDKNFKNMKSFSIAKLNNAVKGKYDKTIENYIKVFGAFDEEENLLQRLETAYNEAEPILNNIEDRCKNINQDKDAVEKIKTLLDALKDIQHFAKLLLCDNDETEIDAEFYNKLHDIWVKLDKITPIYNMVRNYVTKKPYSEEKIKLNFEKSTLLGGWDLNKEKNNLSVILRKDNLYYLGIMKKDNNKIFDSTNIKTDGVCFEKMEYKLLPDPKKMLPKVFFSKKCSKDFNPNDKILEIKENESFKKTSSNFNIEQCRKLIDFYKESINKHKDWQKFNFQFSDTKTYNDINEFYNEVEKQGYKISFCKISEDYINELVKDNKLYLFKIWNKDFSKYSKGTPNTHTLYWKQIFAPENINNVVYKLNGQAEIFFRQASISQKNVIKHLANKPVKNKNIKNEKKESTFSYDLVKDKRFTMDKFHFHVPITINFKAKGINNTNPIVNNLIRQNKIEHIIGIDRGERHLLYLSLIDLKGNIIEQKSLNEIINNYNGNEYKTDYHTLLDDKEKERKDARLSWNTIENIKELKDGYMSQVVHIISQMIVKYNAIVVLEDLNHGFVRGRQKIEKQVYEKFEHKLIDKLNYYVDKNADSNAVGGLYNALQLTNPFDSFEKLGKQSGCLFYIPAWKTSKIDPVTGFINMFTNLKYESVEKSKKFFSKFDDIRYNKEKNRFEFDVSFDKFDSDFVRITQESKLHWTLCSVGQRIELVKENNGYKPNEINLTDAFKSVFNTNKIEINTAKLNREIGKINDTAFFKELMRLMKLLLQMRNSKPNSIEKNDDYIISPVADENGVFFDSSKVEDNGNLPKDADANGAYNIARKGLYVIHQIKQSEDDKKIDFKDFNPRWLKFIQQKLYLND

SEQ ID NO: 105 - protein, 1282 aa, synthetic construct
MGSGKAETNLTELVNLYSLQKTLRFELIPQGKTLENIEKNGILTQDNQRADDYEKVKKLIDEYHKHHIEISLDDCRLEGLEEYKELYEKKDDLKKIQENLRKQIVKSLTENERYKDKRLFSDKLFKEDLPNYLKDREQDKALVKKFEKFTTYFTGFNENRKNMYSSEDKPTSIAYRLIHENLPKFIDNLHIFDKIKETTIKDDFDKIVEKLNKHLKIHIKSFDEIFSIEYFNKTLSQKQIDNYNNIIGGMSFENGTKIQGLNEYINRYNQKQEDKHQKLPCVKTLYKQILSDREKISWIPEQFDDDKQMAESISNLYNEMLPIIKDDLLPLMANIGDYDLSKIFISNDSALTTISQRIFGAYNVYTLAIIEKLKSDKPKSKRQSESKYLDEIDKNFKNMKSFSIAKLNNAVKGKYDKTIENYIKVFGAFDEEENLLQRLETAYNEAEPILNNIEDRCKNINQDKDAVEKIKTLLDALKDIQHFAKLLLCDNDETEIDAEFYNKLHDIWVKLDKITPIYNMVRNYVTKKPYSEEKIKLNFEKSTLLGGWDLNKEKNNLSVILRKDNLYYLGIMKKDNNKIFDSTNIKTDGVCFEKMEYKLLPDPKKMLPKVFFSKKCSKDFNPNDKILEIKENESFKKTSSNFNIEQCRKLIDFYKESINKHKDWQKFNFQFSDTKTYNDINEFYNEVEKQGYKISFCKISEDYINELVKDNKLYLFKIWNKDFSKYSKGTPNTHTLYWKQIFAPENINNVVYKLNGQAEIFFRQASISQKNVIKHLANKPVKNKNIKNEKKESTFSYDLVKDKRFTMDKFHFHVPITINFKAKGINNTNPIVNNLIRQNKIEHIIGIDRGERHLLYLSLIDLKGNIIEQKSLNEIINNYNGNEYKTDYHTLLDDKEKERKDARLSWNTIENIKELKDGYMSQVVHIISQMIVKYNAIVVLEDLNHGFVRGRQKIEKQVYEKFEHKLIDKLNYYVDKNADSNAVGGLYNALQLTNPFDSFEKLGKQSGCLFYIPAWKTSKIDPVTGFINMFTNLKYESVEKSKKFFSKFDDIRYNKEKNRFEFDVSFDKFDSDFVRITQESKLHWTLCSVGQRIELVKENNGYKPNEINLTDAFKSVFNTNKIEINTAKLNREIGKINDTAFFKELMRLMKLLLQMRNSKPNSIEKNDDYIISPVADENGVFFDSSKVEDNGNLPKDADANGAYNIARKGLYVIHQIKQSEDDKKIDFKDFNPRWLKFIQQKLYLNDSRKRTADGSEFESPKKKRKVGSGKPIPNPLLGLDST

SEQ ID NO: 106 - DNA, 3732 bp, unidentified
  source 1..3732: mol_type = unassigned DNA
atgaaagcagaaacaaatctgacagaattagtgaatctgtattcattgcagaaaacacttcgttttgaattaatcccacagggcaaaacattagaaaacattgagaaaaatggtattcttacacaagataaccaaagagcagacgattacgaaaaagtcaaaaaacttattgatgagtatcataagcaccatattgaaataagtcttgacgattgtcgccttgaaggtttagaggaatataaagaactctacgaaaagaaagatgatttgaaaaaaattcaagagaatctacgaaaacaaatcgttaaaagtttaacggagaacgaaaggtataaagacaaacgtctattctctgataaactcttcaaagaagatcttccgaattatctaaaagatagagaacaagacaaagctcttgttaaaaaatttgaaaaattcaccacatattttactggatttaacgaaaacagaaaaaatatgtattcttccgaagacaaacctacctcaattgcttatagattaatccatgaaaatttacctaagtttatagacaatttacatatttttgataaaattaaagaaacaacaatcaaagatgattttgataagattgttgaaaaattaaacaagcatctaaaaattcatatcaaatcatttgacgaaattttctctattgaatatttcaataaaactcttagccaaaaacaaatagacaattataacaatataattggaggaatgtcttttgagaatggtacaaagatacaaggcttaaacgaatatattaatcgttacaatcaaaagcaggaagataaacatcaaaaacttccttgcgtcaaaacactttataagcaaatactcagtgatagagaaaaaatatcgtggattccagaacaatttgatgatgataaacaaatggcagaaagtatttcgaatttgtacaatgaaatgcttccaattattaaagatgatctacttccgctaatggctaatataggcgattatgatcttagcaaaatatttatctccaacgactctgctttaacaacaatatctcaacgaatttttggagcttacaacgtttacactcttgcaataatagaaaaattaaaaagtgataaacctaaatcaaaaagacaatccgagtctaagtatttagacgaaattgacaaaaacttcaaaaatatgaaaagtttcagtattgcaaaactaaacaatgccgtaaaaggcaaatacgataaaacaatagaaaattatatcaaggttttcggggcttttgacgaagaagagaacttgctacaacgattagaaacagcctataacgaagctgagcctatacttaataatatagaagacagatgcaaaaatattaatcaagacaaagatgctgttgaaaagattaaaacattattagatgctttgaaagatattcaacattttgcaaaacttctattatgtgataacgacgaaactgaaatagatgcggagttttataataaattacatgatatatgggtaaaattggacaagataacacctatatataatatggtgagaaattatgttacaaagaaaccttattcagaagaaaaaatcaaattgaattttgaaaaatctacactattaggcggctgggatttgaacaaagaaaaaaataatttatcagttatactccgcaaagataatttgtattacttagggattatgaaaaaagataataacaaaatctttgatagtacaaatatcaaaaccgatggcgtttgttttgagaaaatggaatacaaactacttcctgatccaaagaaaatgctgccaaaggtattcttttcaaaaaaatgttcaaaggactttaacccgaacgacaaaatattagaaattaaggaaaatgaaagtttcaagaaaacaagcagtaatttcaatattgagcaatgtcgtaaattaatagacttctataaagaatctatcaataaacataaagattggcaaaaatttaatttccaattctctgacactaaaacttacaatgacataaacgaattttacaacgaagttgaaaaacaaggttataaaatatctttttgtaaaatttctgaggattatataaatgagttggtgaaagacaataaactttatttgtttaagatttggaacaaagacttttcaaaatatagcaaaggaactccaaatacgcacactctttattggaaacaaatatttgcacctgaaaatatcaacaatgtcgtatataaactaaacggacaagccgaaatattttttaggcaagcaagtatttctcaaaaaaacgttatcaaacatttggcaaacaaacctgttaaaaacaagaatataaaaaacgaaaaaaaggaaagtacgttcagttatgatttagtaaaagataaacgttttactatggataaattccatttccacgtaccgattactattaatttcaaggcaaaaggaataaataataccaatcctattgtcaataatctaattcgtcaaaacaagatagaacatattattggtatagatagaggcgaaaggcatttgctttatctttctcttatagatttgaaaggaaatatcattgaacaaaagtcgttgaatgaaatcataaacaactacaatggcaatgaatataaaacagattaccataccttgcttgatgataaggaaaaagaaagaaaagatgcccgactttcgtggaatactattgaaaatatcaaagaactcaaagacgggtatatgagccaagttgtgcatattatctcacaaatgattgtgaagtacaatgcaatagttgttttggaagaccttaatcatggctttgttcgtggtcgccagaagatagaaaaacaagtttatgaaaaatttgagcataaacttattgataaactaaactattatgtcgataagaatgccgatagcaatgccgttggaggactttacaatgctttgcaactaacaaatccatttgatagttttgaaaaattaggaaaacaaagcggctgtttattctatatccctgcttggaaaacaagtaagattgatcccgttactggatttattaatatgtttacaaatctcaaatacgaatcagtggaaaaatcaaagaagttcttttcaaagtttgacgatattagatacaataaagaaaaaaataggtttgaatttgatgtttcatttgataaattcgatagtgattttgtccgtattacacaggaaagtaaattacattggacgctttgcagtgttggtcagcgtatagaattagtaaaagagaataatggttataaacctaatgaaataaatttaactgatgctttcaaatcagtgtttaatactaataaaatagagataaacactgctaaactgaatagagagattggtaaaatcaatgatacagcgtttttcaaggaacttatgcgtttaatgaaattgttattacaaatgagaaatagtaagccaaattcaatagagaagaacgacgattatattatctctcctgttgcagacgaaaatggagtattctttgacagcagtaaagttgaagacaatggcaatttgccaaaagatgccgatgccaacggagcatacaatattgctcgcaaaggcttgtatgtaatacaccaaataaagcaaagcgaagatgataaaaaaatcgatttcaaagatttcaacccacgttggttaaaattcattcagcaaaaactatatttgaatgattga

SEQ ID NO: 107 - DNA, 3726 bp, synthetic construct
  source 1..3726: mol_type = other DNA
aaggccgagacaaacctgacagaactcgtgaacctgtacagcctgcaaaaaaccctgagatttgagctcatcccccagggcaagacccttgagaacatcgagaagaacggtatcctgacccaggacaatcagagagccgacgactacgagaaggtgaaaaaactgatcgacgagtaccacaagcaccacatcgagatcagcctggacgattgcagactggaaggcctggaagaatacaaggaactgtatgagaagaaggatgacctaaagaaaatccaggaaaacctgagaaagcagatcgtgaagtccctcactgagaacgaacggtacaaggacaaaagactcttctcagataagctgttcaaggaagatctgcctaattacctgaaggacagagaacaggacaaggccctggtaaaaaagttcgagaagttcaccacctacttcaccggcttcaacgaaaaccgcaaaaacatgtacagcagcgaggataagcccaccagcatcgcttatagactgatccacgagaacctgcctaagttcatcgacaacctgcacatctttgataagatcaaggaaaccaccatcaaggacgatttcgataagatcgtggaaaagctgaataaacacctgaagatccacatcaaatccttcgacgagatcttttctattgaatacttcaacaagacactgagtcaaaagcaaatcgacaactacaacaacatcatcggcggaatgagcttcgagaatggcaccaagatccagggcctgaatgagtacatcaacagatacaaccagaaacaagaggacaagcatcaaaagctgccttgcgtgaaaaccctgtacaagcagatcctgagcgacagagagaagatttcctggattcctgaacagttcgatgacgacaaacagatggccgagagcatcagcaatctgtacaacgagatgctgccaatcatcaaggacgacctgctgcctctgatggccaacattggcgactacgacctgagcaaaatcttcatcagcaatgacagcgccctgacaaccatctcgcagcggatcttcggagcttacaacgtgtacaccctggccatcattgagaagctgaagtctgataagcctaagagcaagcggcagtctgagtctaagtacctggacgagatcgacaagaacttcaagaacatgaagtcttttagcatcgccaagctgaacaacgccgtgaagggcaagtatgacaagacaatcgaaaattacatcaaggtgtttggcgcctttgatgaggaggagaatctcctgcagaggctggaaacagcctataacgaggccgagcctatcctgaacaacatcgaggacagatgcaaaaacatcaatcaagacaaggatgccgtggaaaagatcaagaccttactggacgctctgaaagatatccagcactttgccaagttactgctgtgcgacaatgacgaaaccgagattgacgccgagttctacaacaagctgcacgacatctgggtgaagctggacaaaatcacaccaatctacaacatggtgcggaactacgtgaccaagaagccctactctgaagagaaaatcaagctgaacttcgaaaagtctacactgctgggcggctgggatctgaacaaggaaaagaacaatctgagcgtgatcctgagaaaggacaacctgtactacctgggcatcatgaagaaagacaacaacaagatcttcgactccacaaacatcaagaccgacggcgtttgtttcgagaagatggaatataagctgttacctgaccctaaaaagatgctgcccaaggtgttcttctcaaagaaatgcagcaaggatttcaatcctaacgacaagatcctggagatcaaagagaacgaatctttcaagaaaacctctagcaactttaatatcgagcagtgcagaaaactgatcgacttttacaaggagtccatcaataagcacaaagactggcagaaattcaactttcagttcagcgataccaagacctacaacgatatcaacgagttctacaacgaggtggaaaaacagggctacaaaattagcttctgcaagatcagcgaggactacatcaatgagctggttaaggacaacaaactgtacctgtttaagatctggaacaaggatttcagtaagtacagcaaggggacccctaacacccacaccctgtactggaagcagatcttcgcccctgagaacatcaacaacgtcgtgtacaagctgaacggacaggccgagatcttcttcagacaagcatctatctcccagaagaacgtcatcaagcacctagctaataagccagtgaaaaacaagaacatcaagaacgagaagaaggagagcaccttcagctacgatcttgttaaggacaagcggtttacaatggacaagttccacttccacgtgccaatcaccataaactttaaggccaagggcatcaacaacaccaatcctattgtcaacaacctgatccggcagaacaagattgaacacatcatcggcatcgacagaggcgagagacacctgctgtatctgagcctgatcgatctgaagggcaacatcatagaacagaagagcctgaacgagatcatcaacaattacaacggcaatgagtacaagaccgattaccataccttgctggatgacaaggaaaaggagagaaaggatgctagactgagctggaacaccatcgaaaatatcaaggaactgaaagatggctacatgagccaggtggtgcacatcatcagtcagatgatcgtgaaatacaacgccattgtggtcctggaggatctcaaccacggcttcgtgcggggcagacagaagatcgagaagcaggtgtatgaaaaatttgaacacaagctgatcgacaagctgaattactacgtggacaagaatgctgacagcaacgccgtgggaggactgtacaatgccctgcagctgacaaaccccttcgacagcttcgagaagctgggcaagcagagcggctgtctgttttacatccccgcctggaaaacaagtaagatcgatcctgtgaccggattcatcaacatgttcaccaacctgaagtacgaatctgtggaaaagagcaaaaagttcttcagcaagttcgatgacatcagatacaacaaggagaaaaaccgattcgagttcgacgtgtccttcgacaagttcgactccgacttcgtgcggatcacccaggagagcaaactgcattggaccttgtgtagcgtgggccagagaatcgaactggtcaaggaaaacaacggatacaagcctaacgaaatcaacctgacagatgctttcaagagcgtgttcaacacaaacaagatcgagatcaacaccgccaaactgaatcgggaaatcggaaaaatcaacgacacagctttcttcaaggaactgatgcggctgatgaagctgctcctgcagatgagaaacagcaagcccaactccatcgaaaagaacgatgattacatcatcagccctgtggccgatgagaacggcgtgttctttgacagcagcaaagtggaggacaatggcaacctgccaaaggacgccgatgccaacggcgcctacaacatcgccaggaagggcctgtatgtgatccaccagattaagcagtctgaggacgacaagaagatcgactttaaggacttcaaccccagatggctgaagttcatccagcagaagctgtacctgaacgat

SEQ ID NO: 108 - DNA, 3849 bp, synthetic construct
  source 1..3849: mol_type = other DNA
atgggctccggaaaggccgagacaaacctgacagaactcgtgaacctgtacagcctgcaaaaaaccctgagatttgagctcatcccccagggcaagacccttgagaacatcgagaagaacggtatcctgacccaggacaatcagagagccgacgactacgagaaggtgaaaaaactgatcgacgagtaccacaagcaccacatcgagatcagcctggacgattgcagactggaaggcctggaagaatacaaggaactgtatgagaagaaggatgacctaaagaaaatccaggaaaacctgagaaagcagatcgtgaagtccctcactgagaacgaacggtacaaggacaaaagactcttctcagataagctgttcaaggaagatctgcctaattacctgaaggacagagaacaggacaaggccctggtaaaaaagttcgagaagttcaccacctacttcaccggcttcaacgaaaaccgcaaaaacatgtacagcagcgaggataagcccaccagcatcgcttatagactgatccacgagaacctgcctaagttcatcgacaacctgcacatctttgataagatcaaggaaaccaccatcaaggacgatttcgataagatcgtggaaaagctgaataaacacctgaagatccacatcaaatccttcgacgagatcttttctattgaatacttcaacaagacactgagtcaaaagcaaatcgacaactacaacaacatcatcggcggaatgagcttcgagaatggcaccaagatccagggcctgaatgagtacatcaacagatacaaccagaaacaagaggacaagcatcaaaagctgccttgcgtgaaaaccctgtacaagcagatcctgagcgacagagagaagatttcctggattcctgaacagttcgatgacgacaaacagatggccgagagcatcagcaatctgtacaacgagatgctgccaatcatcaaggacgacctgctgcctctgatggccaacattggcgactacgacctgagcaaaatcttcatcagcaatgacagcgccctgacaaccatctcgcagcggatcttcggagcttacaacgtgtacaccctggccatcattgagaagctgaagtctgataagcctaagagcaagcggcagtctgagtctaagtacctggacgagatcgacaagaacttcaagaacatgaagtcttttagcatcgccaagctgaacaacgccgtgaagggcaagtatgacaagacaatcgaaaattacatcaaggtgtttggcgcctttgatgaggaggagaatctcctgcagaggctggaaacagcctataacgaggccgagcctatcctgaacaacatcgaggacagatgcaaaaacatcaatcaagacaaggatgccgtggaaaagatcaagaccttactggacgctctgaaagatatccagcactttgccaagttactgctgtgcgacaatgacgaaaccgagattgacgccgagttctacaacaagctgcacgacatctgggtgaagctggacaaaatcacaccaatctacaacatggtgcggaactacgtgaccaagaagccctactctgaagagaaaatcaagctgaacttcgaaaagtctacactgctgggcggctgggatctgaacaaggaaaagaacaatctgagcgtgatcctgagaaaggacaacctgtactacctgggcatcatgaagaaagacaacaacaagatcttcgactccacaaacatcaagaccgacggcgtttgtttcgagaagatggaatataagctgttacctgaccctaaaaagatgctgcccaaggtgttcttctcaaagaaatgcagcaaggatttcaatcctaacgacaagatcctggagatcaaagagaacgaatctttcaagaaaacctctagcaactttaatatcgagcagtgcagaaaactgatcgacttttacaaggagtccatcaataagcacaaagactggcagaaattcaactttcagttcagcgataccaagacctacaacgatatcaacgagttctacaacgaggtggaaaaacagggctacaaaattagcttctgcaagatcagcgaggactacatcaatgagctggttaaggacaacaaactgtacctgtttaagatctggaacaaggatttcagtaagtacagcaaggggacccctaacacccacaccctgtactggaagcagatcttcgcccctgagaacatcaacaacgtcgtgtacaagctgaacggacaggccgagatcttcttcagacaagcatctatctcccagaagaacgtcatcaagcacctagctaataagccagtgaaaaacaagaacatcaagaacgagaagaaggagagcaccttcagctacgatcttgttaaggacaagcggtttacaatggacaagttccacttccacgtgccaatcaccataaactttaaggccaagggcatcaacaacaccaatcctattgtcaacaacctgatccggcagaacaagattgaacacatcatcggcatcgacagaggcgagagacacctgctgtatctgagcctgatcgatctgaagggcaacatcatagaacagaagagcctgaacgagatcatcaacaattacaacggcaatgagtacaagaccgattaccataccttgctggatgacaaggaaaaggagagaaaggatgctagactgagctggaacaccatcgaaaatatcaaggaactgaaagatggctacatgagccaggtggtgcacatcatcagtcagatgatcgtgaaatacaacgccattgtggtcctggaggatctcaaccacggcttcgtgcggggcagacagaagatcgagaagcaggtgtatgaaaaatttgaacacaagctgatcgacaagctgaattactacgtggacaagaatgctgacagcaacgccgtgggaggactgtacaatgccctgcagctgacaaaccccttcgacagcttcgagaagctgggcaagcagagcggctgtctgttttacatccccgcctggaaaacaagtaagatcgatcctgtgaccggattcatcaacatgttcaccaacctgaagtacgaatctgtggaaaagagcaaaaagttcttcagcaagttcgatgacatcagatacaacaaggagaaaaaccgattcgagttcgacgtgtccttcgacaagttcgactccgacttcgtgcggatcacccaggagagcaaactgcattggaccttgtgtagcgtgggccagagaatcgaactggtcaaggaaaacaacggatacaagcctaacgaaatcaacctgacagatgctttcaagagcgtgttcaacacaaacaagatcgagatcaacaccgccaaactgaatcgggaaatcggaaaaatcaacgacacagctttcttcaaggaactgatgcggctgatgaagctgctcctgcagatgagaaacagcaagcccaactccatcgaaaagaacgatgattacatcatcagccctgtggccgatgagaacggcgtgttctttgacagcagcaaagtggaggacaatggcaacctgccaaaggacgccgatgccaacggcgcctacaacatcgccaggaagggcctgtatgtgatccaccagattaagcagtctgaggacgacaagaagatcgactttaaggacttcaaccccagatggctgaagttcatccagcagaagctgtacctgaacgattctagaaagcggacagcagacggctccgaatttgaaagccctaagaaaaagagaaaggtgggatccggcaaacctatccccaatcccctgctgggcctggacagcacctga

SEQ ID NO: 109 - protein, 1248 aa, unidentified
DMKSLNSFQNQYSLSKTLRFQLIPQGKTLDNINESRILEEDQHRSESYKLVKKIIDDYHKAYIEQALGSFELKIASDSKNDSLEEFYSQYIAERKEDKAKKLFEKTQDNLRKQISKKLKQGEAYKRLFGKELIQEDLLEFVATDPEADSKKRLIEEFKDFTTYFIGFHENRKNMYAEEAQSTAIAYRIIHENLPKFIDNIRTFEELAKSSIADVLPQVYEDFKAYLKVESVKELFSLDYFNTVLTQKQLDIYNAVIGGKSLDENSRIQGLNEYINLYNQQHKDKKLPFLKPLFKQILSDRNSLSWLPEAFDNDKQVLQAVHDCYTSLLESVFHKDGLQQLLQSLPTYNLKGIYLRNDLSMTNVSQKLLGDWGAITRAVKEKLQKENPAKKRESDEAYQERINKIFKQAGSYSLDYINQALEATDQTNIKVEDYFINMGVDNEQKEPLFQRVAQAYNQASDLLEKEYPANKNLMQDKESIEHIKFLLDNLKAVQHFIKPLLGDGNEADKDNRFYGELTALWNELDQVTRLYNKVRNYMTRKPYSVDKIKINFKNSTLLNGWDRNKERDNTAVILRKDGKFYLAIMHKEHNKVFEKFPVGTKDSDFEKMEYKLLPGANKMLPKVFFSKSRIDEFKPSAELLQKYQMGTHKKGELFSLNDCHSLIDFFKASIEKHDDWKQFNFHFSPTSSYEDLSGFYREVEQQGYKLTFKSVDADYINKMVDEGKIFLFQIYNKDFSEHSKGTPNLHTLYWKMLFDERNLQNVVYKLNGEAEVFFRKKSLTYTRPTHPKKEPIKNKNVQNAKKESIFDYDLIKNKRFTVDSFQFHVPITMNFKSEGRSNLNERVNEFLRQNNDAHIIGIDRGERHLLYLVVIDRHGNIVEQFSLNSIINEYQGNTYATNYHDLLDKREKEREEARESWQSIENIKELKEGYLSQVVHKIADLMVKYHAIVVLEDLNMGFMRGRQKVEKQVYQKFEKMLIDKLNYLVDKKQDAETDGGLLKAYQLTNQFESFQKLGKQSGFLFYVPAWNTSKIDPCTGFTNLLDTRYESIEKAKKFFQTFNAIRYNAAQGYFEFELDYNKFNKRADGTQTLWTLCTYGPRIETLRSTEDNNKWTSKEVDLTDELKKHFYHYGIKLDADLKEAIGQQTDKPFFTNLLHLLKLTLQMRNSKIGTEVDYLISPIRNEDGTFYDSRQGNKSLPANADANGAYNIARKGLWVINQIKQTPQDQKPKLAITNKEWLQFAQEKPYLKD

SEQ ID NO: 110 - protein, 1249 aa, unidentified
MDMKSLNSFQNQYSLSKTLRFQLIPQGKTLDNINESRILEEDQHRSESYKLVKKIIDDYHKAYIEQALGSFELKIASDSKNDSLEEFYSQYIAERKEDKAKKLFEKTQDNLRKQISKKLKQGEAYKRLFGKELIQEDLLEFVATDPEADSKKRLIEEFKDFTTYFIGFHENRKNMYAEEAQSTAIAYRIIHENLPKFIDNIRTFEELAKSSIADVLPQVYEDFKAYLKVESVKELFSLDYFNTVLTQKQLDIYNAVIGGKSLDENSRIQGLNEYINLYNQQHKDKKLPFLKPLFKQILSDRNSLSWLPEAFDNDKQVLQAVHDCYTSLLESVFHKDGLQQLLQSLPTYNLKGIYLRNDLSMTNVSQKLLGDWGAITRAVKEKLQKENPAKKRESDEAYQERINKIFKQAGSYSLDYINQALEATDQTNIKVEDYFINMGVDNEQKEPLFQRVAQAYNQASDLLEKEYPANKNLMQDKESIEHIKFLLDNLKAVQHFIKPLLGDGNEADKDNRFYGELTALWNELDQVTRLYNKVRNYMTRKPYSVDKIKINFKNSTLLNGWDRNKERDNTAVILRKDGKFYLAIMHKEHNKVFEKFPVGTKDSDFEKMEYKLLPGANKMLPKVFFSKSRIDEFKPSAELLQKYQMGTHKKGELFSLNDCHSLIDFFKASIEKHDDWKQFNFHFSPTSSYEDLSGFYREVEQQGYKLTFKSVDADYINKMVDEGKIFLFQIYNKDFSEHSKGTPNLHTLYWKMLFDERNLQNVVYKLNGEAEVFFRKKSLTYTRPTHPKKEPIKNKNVQNAKKESIFDYDLIKNKRFTVDSFQFHVPITMNFKSEGRSNLNERVNEFLRQNNDAHIIGIDRGERHLLYLVVIDRHGNIVEQFSLNSIINEYQGNTYATNYHDLLDKREKEREEARESWQSIENIKELKEGYLSQVVHKIADLMVKYHAIVVLEDLNMGFMRGRQKVEKQVYQKFEKMLIDKLNYLVDKKQDAETDGGLLKAYQLTNQFESFQKLGKQSGFLFYVPAWNTSKIDPCTGFTNLLDTRYESIEKAKKFFQTFNAIRYNAAQGYFEFELDYNKFNKRADGTQTLWTLCTYGPRIETLRSTEDNNKWTSKEVDLTDELKKHFYHYGIKLDADLKEAIGQQTDKPFFTNLLHLLKLTLQMRNSKIGTEVDYLISPIRNEDGTFYDSRQGNKSLPANADANGAYNIARKGLWVINQIKQTPQDQKPKLAITNKEWLQFAQEKPYLKD

SEQ ID NO: 111 - protein, 1288 aa, synthetic construct
MGSGDMKSLNSFQNQYSLSKTLRFQLIPQGKTLDNINESRILEEDQHRSESYKLVKKIIDDYHKAYIEQALGSFELKIASDSKNDSLEEFYSQYIAERKEDKAKKLFEKTQDNLRKQISKKLKQGEAYKRLFGKELIQEDLLEFVATDPEADSKKRLIEEFKDFTTYFIGFHENRKNMYAEEAQSTAIAYRIIHENLPKFIDNIRTFEELAKSSIADVLPQVYEDFKAYLKVESVKELFSLDYFNTVLTQKQLDIYNAVIGGKSLDENSRIQGLNEYINLYNQQHKDKKLPFLKPLFKQILSDRNSLSWLPEAFDNDKQVLQAVHDCYTSLLESVFHKDGLQQLLQSLPTYNLKGIYLRNDLSMTNVSQKLLGDWGAITRAVKEKLQKENPAKKRESDEAYQERINKIFKQAGSYSLDYINQALEATDQTNIKVEDYFINMGVDNEQKEPLFQRVAQAYNQASDLLEKEYPANKNLMQDKESIEHIKFLLDNLKAVQHFIKPLLGDGNEADKDNRFYGELTALWNELDQVTRLYNKVRNYMTRKPYSVDKIKINFKNSTLLNGWDRNKERDNTAVILRKDGKFYLAIMHKEHNKVFEKFPVGTKDSDFEKMEYKLLPGANKMLPKVFFSKSRIDEFKPSAELLQKYQMGTHKKGELFSLNDCHSLIDFFKASIEKHDDWKQFNFHFSPTSSYEDLSGFYREVEQQGYKLTFKSVDADYINKMVDEGKIFLFQIYNKDFSEHSKGTPNLHTLYWKMLFDERNLQNVVYKLNGEAEVFFRKKSLTYTRPTHPKKEPIKNKNVQNAKKESIFDYDLIKNKRFTVDSFQFHVPITMNFKSEGRSNLNERVNEFLRQNNDAHIIGIDRGERHLLYLVVIDRHGNIVEQFSLNSIINEYQGNTYATNYHDLLDKREKEREEARESWQSIENIKELKEGYLSQVVHKIADLMVKYHAIVVLEDLNMGFMRGRQKVEKQVYQKFEKMLIDKLNYLVDKKQDAETDGGLLKAYQLTNQFESFQKLGKQSGFLFYVPAWNTSKIDPCTGFTNLLDTRYESIEKAKKFFQTFNAIRYNAAQGYFEFELDYNKFNKRADGTQTLWTLCTYGPRIETLRSTEDNNKWTSKEVDLTDELKKHFYHYGIKLDADLKEAIGQQTDKPFFTNLLHLLKLTLQMRNSKIGTEVDYLISPIRNEDGTFYDSRQGNKSLPANADANGAYNIARKGLWVINQIKQTPQDQKPKLAITNKEWLQFAQEKPYLKDSRKRTADGSEFESPKKKRKVGSGKPIPNPLLGLDST

SEQ ID NO: 112 - DNA, 3750 bp, unidentified
  source 1..3750: mol_type = unassigned DNA
atggatatgaaaagtttaaacagctttcagaaccagtattccctatccaagaccctccggtttcagctaataccccagggtaaaactttggataacattaacgagagcagaatattggaggaagaccaacaccgaagcgaaagctacaagttggtcaagaaaatcattgacgactatcacaaggcctacatcgaacaagccctgggcagtttcgaactcaaaattgccagtgactctaaaaacgattcgttagaggagttctactcgcagtatattgccgaacggaaagaagataaagccaaaaaacttttcgaaaagacgcaagacaacttgcgaaagcaaatctccaagaaattaaagcagggcgaagcctacaagcggttgtttggcaaggaactcattcaagaagacctgctggagtttgtagctaccgaccctgaggctgatagcaaaaagcgtctgattgaagaattcaaggacttcaccacctactttatcggattccacgagaaccgaaagaacatgtatgctgaggaagcccaatccacagcaattgcctaccgcatcattcacgagaacctgccgaagttcattgataacatacgcaccttcgaagaacttgctaaaagttccattgccgacgtcctgccacaggtttatgaagatttcaaagcgtacttaaaggtcgaatcggtcaaagaacttttcagtctggactatttcaataccgtcttgacccaaaagcagcttgacatttacaatgcggttatcggcggtaagtcgttagatgagaacagccgcatccaggggctcaacgagtatatcaacctgtacaaccagcagcacaaggacaaaaagttacccttcttaaaacccttgttcaagcaaattctgagcgaccgcaacagcctttcgtggttgcccgaagctttcgacaatgacaagcaggtacttcaggctgtacacgactgctacacctcgctattggagagcgtattccacaaagacggcctgcaacagttgctacagtcactgcctacctacaacctgaagggcatttacctgcgcaacgacctttccatgaccaacgtttctcaaaaactattgggcgattggggagctattacacgtgccgttaaagaaaaactacaaaaagaaaatcctgccaaaaaacgagagtcggacgaagcctaccaagaacgcatcaacaagatattcaagcaagccggcagctactctttagattacatcaaccaagcgctcgaagcaacagaccagaccaatatcaaagtcgaagactacttcatcaacatgggcgtagacaacgagcaaaaagagcccctgttccagcgtgtagcgcaagcctacaatcaggccagcgatttgcttgaaaaggaatatcccgcaaacaaaaatctgatgcaggataaagaaagcatcgagcacatcaaattcttgctcgataacctcaaagccgttcaacactttataaagcccctgctcggcgatggtaacgaggctgataaagataatcgtttttacggagaacttacagcgctgtggaacgaattagaccaggtaacgcgcctgtataacaaggtgcgaaactacatgacccgcaagccctactcggttgataaaatcaagattaactttaagaactcaactctacttaatggctgggacagaaataaggaacgtgacaataccgctgttattctgcgcaaagacggcaagttctatctggccattatgcataaagaacacaataaggtgttcgaaaaattcccggtcggaacaaaggattctgacttcgagaaaatggagtataagttacttccgggcgccaataaaatgcttccgaaggttttcttctctaaatcgcgtatcgatgagtttaagcccagcgccgaacttctccaaaagtaccagatgggtacccacaaaaagggcgaactcttcagtctgaacgactgccattctctgattgacttctttaaggcttctattgaaaagcatgacgattggaaacagtttaacttccatttctcacccacttcgagctacgaagacttgagcggattttacagagaggttgaacagcaggggtacaaactgaccttcaaatccgttgacgccgactatatcaacaaaatggttgacgagggcaaaatctttctcttccagatttacaataaagacttctcggaacatagcaaaggcacccccaacctgcatacgctctactggaaaatgctctttgacgaacgcaacctgcagaacgtggtctacaaactgaacggcgaggccgaagtcttcttccggaagaagagtcttacctacacccgtcctacgcaccccaagaaagagcctatcaagaacaagaacgttcagaatgccaaaaaggaaagcatcttcgactacgacctgattaaaaacaaacgctttacggtcgactccttccagttccacgttcccatcacgatgaacttcaagagcgaaggacgctccaacctgaacgagcgggtcaacgagtttttacgccagaacaacgatgcccacatcattggcattgaccggggcgaacgccatttgctctacctggtggttattgaccggcacggaaacattgtggaacaattttcgctcaactctatcatcaacgaatatcagggtaatacgtacgccaccaactaccacgacttgttggataagcgcgaaaaggaaagagaggaagcacgcgaaagctggcagagtattgagaatattaaagaactgaaagaaggatacttgagccaggtggtgcataaaattgccgacctcatggtaaagtatcatgccatcgtggtgctcgaagacttgaatatgggcttcatgcgcggacgccagaaggtagaaaagcaggtctatcagaagtttgaaaaaatgctgatagacaagttaaactatctggttgacaagaagcaagatgccgaaaccgacggcggtctgctcaaggcataccaactgaccaaccagttcgaaagtttccagaagttaggcaagcagagcggtttcctcttctatgtgcctgcctggaacaccagcaaaattgacccctgcaccggatttaccaacctgctcgacactcgatacgagagcatcgaaaaggccaaaaagttctttcaaactttcaatgccatccgctacaatgctgcgcaggggtactttgagttcgaactggattacaataaattcaacaagcgggccgatggtacacaaaccctatggacgctctgcacctacggcccacgcatcgaaacactccgaagcaccgaggataataacaagtggacaagcaaagaggttgatttgaccgacgaattgaaaaagcacttctaccactatggcattaagctggatgccgacctgaaggaagccatcggccaacaaaccgacaaacctttcttcaccaacttgctccatctgctcaaactaacactgcaaatgcgaaacagcaaaatcggcacggaggttgactacctcatttcgccaattcgcaatgaagacggaacgttctacgacagccgacaaggcaacaaatcattgcctgccaatgccgatgccaatggtgcctacaacattgcccgaaagggtttatgggtaattaaccagataaaacaaacacctcaagaccaaaagcccaagttagctattaccaacaaggaatggctgcaatttgctcaagagaagccctaccttaaggattga

SEQ ID NO: 113 - DNA, 3744 bp, synthetic construct
  source 1..3744: mol_type = other DNA
gacatgaagagcctgaactcttttcagaaccaatactctctgagcaaaaccctgcggttccagctgatccctcagggcaagacactggataatatcaacgagagcagaatcctggaagaggatcagcacagaagcgagtcatataaactggtgaagaagatcattgacgactatcacaaggcctacatcgagcaggccctgggcagcttcgagctgaaaattgcctccgatagcaagaacgacagcctggaggagttctactctcagtacattgcggagagaaaggaggacaaggccaagaagctgttcgaaaagacccaggacaatctgagaaagcagatctccaagaagctgaaacagggtgaagcctacaaacggctgttcggcaaagaactgatccaggaggacctgctggagttcgtggccacagatcctgaggccgactctaagaagagactgatcgaagagttcaaggactttaccacctacttcatcggatttcacgaaaatagaaagaacatgtacgccgaggaggctcagagcacagctattgcctacagaatcatccacgagaacctgccaaagtttatcgataatatcagaaccttcgaggaactggccaagagcagcatcgccgacgtgctgccccaggtctacgaggactttaaggcctacctgaaggtggaaagcgtgaaagaactgttctctctggattatttcaacaccgtgctgacacagaaacaactggacatctacaatgccgtgatcggcggaaaaagcctggacgagaacagcagaatccagggcctgaacgagtacatcaacctctacaaccagcagcataaggacaagaagctgcctttcctgaagcccctgttcaagcaaatcctgtccgatagaaacagcctgtcctggctgcctgaggccttcgacaacgacaagcaggtgctgcaggccgtgcacgactgctacaccagcctgctggaatctgtgttccacaaggacggcctgcaacagctgctgcagagcctcccaacctacaacttaaaaggcatctacctgcggaacgaccttagcatgaccaatgtgtcccagaagctgctgggcgattggggcgctatcaccagagccgtgaaggaaaagctgcagaaggaaaaccctgccaagaagagagagtcggacgaggcctaccaggagcggatcaacaagatcttcaagcaggccggctcatattcactggattacatcaaccaggccctcgaagccacagaccagacaaacatcaaagtggaggactactttatcaacatgggcgtggataatgagcagaaagagcctctgtttcaaagggtggcccaggcctataaccaggccagcgacctgctggaaaaagaataccccgctaacaagaatctgatgcaggacaaggagagcatcgagcacatcaaattcctgctcgacaaccttaaggccgtgcagcacttcatcaagcctctgctgggagatggcaacgaagccgacaaggacaacagattctacggcgagctaaccgccctgtggaacgaacttgaccaggtgacccgcctgtacaacaaggtgcggaattacatgaccaggaagccttacagcgtggacaagatcaaaatcaacttcaagaacagcaccctgctgaacggatgggacagaaacaaggaacgggacaacacagctgtcatcctgagaaaggacggcaagttctacctcgccatcatgcacaaggaacacaacaaggtctttgagaagtttcctgtgggcactaaggattctgacttcgagaagatggaatacaagctgctgcccggcgccaacaagatgctgcctaaggttttctttagcaagagcagaatcgacgagttcaagccatctgccgagctgctgcagaagtaccagatgggaactcacaagaagggagaactgttcagcctgaacgattgccacagcctgatcgacttcttcaaagcctctatcgagaagcacgatgattggaagcagttcaacttccatttcagccctaccagcagctacgaggacctgagcggcttctaccgggaggtggaacagcagggctacaagctgaccttcaagagcgtggacgctgattacatcaataagatggtcgatgaaggcaaaatcttcctgttccagatctacaacaaggattttagcgagcacagcaagggcacacctaacctgcacaccctgtactggaagatgctgttcgacgagagaaacctgcagaacgtggtgtacaagctgaacggcgaagctgaggtgttctttcggaagaagagcctgacctacacacgccccacccaccctaagaaggagcctatcaagaacaaaaacgtgcagaacgctaaaaaggaaagcatcttcgattacgacctgatcaagaacaaaagattcacagtggattctttccagttccacgtgcctatcacaatgaacttcaaatctgagggcagaagcaacctgaatgagagggtgaacgagttcctgagacaaaacaacgatgcccacatcatcggaatcgacagaggcgaaaggcatctgctgtacctggtggtgattgatagacacggcaacatcgtggaacaatttagcctgaacagcataatcaatgagtaccaaggcaatacctacgccacaaactatcacgacctcctggacaagagagagaaggagcgggaagaggccagagagtcctggcagtctatcgagaacatcaaggagctcaaagaaggctacctgagtcaggtggtgcacaaaatcgccgacctgatggtgaagtatcacgccatcgtggtgctggaggacctgaacatgggcttcatgagaggccgacagaaggtagagaagcaggtttaccagaaattcgagaagatgctgattgacaagctgaactatctggtggacaaaaagcaagatgctgaaaccgacggcggcctgctcaaggcctaccaactgaccaaccagttcgagagcttccagaagctgggcaaacagtctggcttcctgttttacgtgcccgcctggaacaccagcaagatcgatccctgtacaggcttcaccaacctgctggacacccgatacgagagcatcgaaaaagcaaagaagttcttccaaacattcaacgccataagatacaacgctgctcaggggtattttgagttcgagctcgactacaacaagtttaacaagcgggccgatggcacccagaccctgtggacactgtgcacctacggacctagaatcgaaaccctgcggagcacagaggacaacaacaagtggaccagcaaagaggtggacctgacagacgagctgaagaaacacttctaccactacggcatcaagttggatgccgacctgaaagaggccatcggccagcaaacagacaagcccttcttcaccaacctgctgcacctgctgaagctgacactgcagatgagaaacagcaagatcggaaccgaggtggactacctgattagccccatcagaaacgaagatggcaccttctacgacagcagacagggaaacaagagcctgcctgctaatgcggacgccaatggcgcctacaacatcgctagaaaaggcctctgggtcatcaaccagatcaaacagacccctcaggatcagaaacctaagctggccatcaccaataaggagtggctgcagttcgcccaggagaaaccatacctgaaagac

SEQ ID NO: 114 - DNA, 3867 bp, synthetic construct
  source 1..3867: mol_type = other DNA
atgggctccggagacatgaagagcctgaactcttttcagaaccaatactctctgagcaaaaccctgcggttccagctgatccctcagggcaagacactggataatatcaacgagagcagaatcctggaagaggatcagcacagaagcgagtcatataaactggtgaagaagatcattgacgactatcacaaggcctacatcgagcaggccctgggcagcttcgagctgaaaattgcctccgatagcaagaacgacagcctggaggagttctactctcagtacattgcggagagaaaggaggacaaggccaagaagctgttcgaaaagacccaggacaatctgagaaagcagatctccaagaagctgaaacagggtgaagcctacaaacggctgttcggcaaagaactgatccaggaggacctgctggagttcgtggccacagatcctgaggccgactctaagaagagactgatcgaagagttcaaggactttaccacctacttcatcggatttcacgaaaatagaaagaacatgtacgccgaggaggctcagagcacagctattgcctacagaatcatccacgagaacctgccaaagtttatcgataatatcagaaccttcgaggaactggccaagagcagcatcgccgacgtgctgccccaggtctacgaggactttaaggcctacctgaaggtggaaagcgtgaaagaactgttctctctggattatttcaacaccgtgctgacacagaaacaactggacatctacaatgccgtgatcggcggaaaaagcctggacgagaacagcagaatccagggcctgaacgagtacatcaacctctacaaccagcagcataaggacaagaagctgcctttcctgaagcccctgttcaagcaaatcctgtccgatagaaacagcctgtcctggctgcctgaggccttcgacaacgacaagcaggtgctgcaggccgtgcacgactgctacaccagcctgctggaatctgtgttccacaaggacggcctgcaacagctgctgcagagcctcccaacctacaacttaaaaggcatctacctgcggaacgaccttagcatgaccaatgtgtcccagaagctgctgggcgattggggcgctatcaccagagccgtgaaggaaaagctgcagaaggaaaaccctgccaagaagagagagtcggacgaggcctaccaggagcggatcaacaagatcttcaagcaggccggctcatattcactggattacatcaaccaggccctcgaagccacagaccagacaaacatcaaagtggaggactactttatcaacatgggcgtggataatgagcagaaagagcctctgtttcaaagggtggcccaggcctataaccaggccagcgacctgctggaaaaagaataccccgctaacaagaatctgatgcaggacaaggagagcatcgagcacatcaaattcctgctcgacaaccttaaggccgtgcagcacttcatcaagcctctgctgggagatggcaacgaagccgacaaggacaacagattctacggcgagctaaccgccctgtggaacgaacttgaccaggtgacccgcctgtacaacaaggtgcggaattacatgaccaggaagccttacagcgtggacaagatcaaaatcaacttcaagaacagcaccctgctgaacggatgggacagaaacaaggaacgggacaacacagctgtcatcctgagaaaggacggcaagttctacctcgccatcatgcacaaggaacacaacaaggtctttgagaagtttcctgtgggcactaaggattctgacttcgagaagatggaatacaagctgctgcccggcgccaacaagatgctgcctaaggttttctttagcaagagcagaatcgacgagttcaagccatctgccgagctgctgcagaagtaccagatgggaactcacaagaagggagaactgttcagcctgaacgattgccacagcctgatcgacttcttcaaagcctctatcgagaagcacgatgattggaagcagttcaacttccatttcagccctaccagcagctacgaggacctgagcggcttctaccgggaggtggaacagcagggctacaagctgaccttcaagagcgtggacgctgattacatcaataagatggtcgatgaaggcaaaatcttcctgttccagatctacaacaaggattttagcgagcacagcaagggcacacctaacctgcacaccctgtactggaagatgctgttcgacgagagaaacctgcagaacgtggtgtacaagctgaacggcgaagctgaggtgttctttcggaagaagagcctgacctacacacgccccacccaccctaagaaggagcctatcaagaacaaaaacgtgcagaacgctaaaaaggaaagcatcttcgattacgacctgatcaagaacaaaagattcacagtggattctttccagttccacgtgcctatcacaatgaacttcaaatctgagggcagaagcaacctgaatgagagggtgaacgagttcctgagacaaaacaacgatgcccacatcatcggaatcgacagaggcgaaaggcatctgctgtacctggtggtgattgatagacacggcaacatcgtggaacaatttagcctgaacagcataatcaatgagtaccaaggcaatacctacgccacaaactatcacgacctcctggacaagagagagaaggagcgggaagaggccagagagtcctggcagtctatcgagaacatcaaggagctcaaagaaggctacctgagtcaggtggtgcacaaaatcgccgacctgatggtgaagtatcacgccatcgtggtgctggaggacctgaacatgggcttcatgagaggccgacagaaggtagagaagcaggtttaccagaaattcgagaagatgctgattgacaagctgaactatctggtggacaaaaagcaagatgctgaaaccgacggcggcctgctcaaggcctaccaactgaccaaccagttcgagagcttccagaagctgggcaaacagtctggcttcctgttttacgtgcccgcctggaacaccagcaagatcgatccctgtacaggcttcaccaacctgctggacacccgatacgagagcatcgaaaaagcaaagaagttcttccaaacattcaacgccataagatacaacgctgctcaggggtattttgagttcgagctcgactacaacaagtttaacaagcgggccgatggcacccagaccctgtggacactgtgcacctacggacctagaatcgaaaccctgcggagcacagaggacaacaacaagtggaccagcaaagaggtggacctgacagacgagctgaagaaacacttctaccactacggcatcaagttggatgccgacctgaaagaggccatcggccagcaaacagacaagcccttcttcaccaacctgctgcacctgctgaagctgacactgcagatgagaaacagcaagatcggaaccgaggtggactacctgattagccccatcagaaacgaagatggcaccttctacgacagcagacagggaaacaagagcctgcctgctaatgcggacgccaatggcgcctacaacatcgctagaaaaggcctctgggtcatcaaccagatcaaacagacccctcaggatcagaaacctaagctggccatcaccaataaggagtggctgcagttcgcccaggagaaaccatacctgaaagactctagaaagcggacagcagacggctccgaatttgaaagccctaagaaaaagagaaaggtgggatccggcaaacctatccccaatcccctgctgggcctggacagcacctga

SEQ ID NO: 115 - protein, 1251 aa, unidentified
KAFENFTGLYPLSKTLRFELKPIGKTLEYIEKHGILDKDKHRANSYVKVKDIIDRYHKQFIEDSLSDSDFKLKYENKGKKESLEEYFYYYKLRNRDDKQKKDFDEIQKNLRKQIASQLKKQDRFKRIDKKELIKEDLLEFVSDDNERNLINEFKDFTTYFTGFHENRQNMYSDEAKSTAIAYRLIHENLPKFIDNISVFERVAATDVADCFAQIYSDFEEYLNVNDISEIFRLDYYTEILTQTQIDAYNLIIGGRSEGNIKIKGLNEYINLYNQQQKDKSQRLPKLKSLFKQILSDRNAISWLPESFENDNQLLEKLESCYQSFNETYDDKKSIFVRFRELLLTISDYEMDKIFLRNDLQLTDISQKMFGSYSIISRSLLEDLKRGTSRKSKKETDESFEERLRNIIKNQDSFAIGTIDSSLQQMDVEEYKKSICDYFPNLSVDDKGDDIFDRIVKAYSEVKDLLNSPYPSDKNLAQEDDDIDKIKNLLESMKDLQKFVKPLCGKGNESDKDERFYGEFTALYEELDKITPLYNMVRNYLTRKPYSTEKIKLNFDNAQLLNGWDLNKESDNTSVILRKDGLYYLAIMNKKHNKVFEKNKLQSDGVCFEKMEYKLLPGANKMLPKVFFSKSRIDEFGPSQRLLDSYQNETHKKGDKFNIEDCHELIDFFKRSIDKHEDWSKFSFSFSDTKTYEDLSGFYREVEHQGYILSFVNVSVDYVNSLVDEGKIYLFQIYNKDFSPFSKGTPNMHTLYWKMLFDEENLKDVVYKLNGQAEVFFRKSSIKYDKPTHPANLPIDNKNVSNHKKRSVFEYDLVKDKRYTVDKFQFHVPVTINFKSDGNGNINPLVNDYIKKSDDLHVIGIDRGERHLLYLTVIDMKGNIKKQFSLNEIVNEYKGNTYSTNYHDLLEKREDKRDKERKEWKTIETIKELKEGYLSQVIHKITELMVEYNAIIVLEDLNLGFMRGRQKVEKSVYQKFEKMLIDKLNYLADKKKEPEDLGGVLKAYQLANKFESFQKMGKQSGFLFYTQAWNTSKIDPVTGFVNLFDTHYENILKSKNFFSKFDLIKYNSDKDWFEFSFDYNNFTTKAEGTKTKWTLCTFGNRIISFRNPDNNMQWDGKEINLTEEFKLFFEKFGININSDLHAEILKQDKKDFFEGLLHLLKLTLQMRNSKTRTDIDYMQSPVADENGVLYNSNKCGKSLPENADANGAYNIARKGLMIIDKIKKSDNLNKIDLTISNKEWLVFAQNKPYLKN

SEQ ID NO: 116 - protein, 1252 aa, unidentified
MKAFENFTGLYPLSKTLRFELKPIGKTLEYIEKHGILDKDKHRANSYVKVKDIIDRYHKQFIEDSLSDSDFKLKYENKGKKESLEEYFYYYKLRNRDDKQKKDFDEIQKNLRKQIASQLKKQDRFKRIDKKELIKEDLLEFVSDDNERNLINEFKDFTTYFTGFHENRQNMYSDEAKSTAIAYRLIHENLPKFIDNISVFERVAATDVADCFAQIYSDFEEYLNVNDISEIFRLDYYTEILTQTQIDAYNLIIGGRSEGNIKIKGLNEYINLYNQQQKDKSQRLPKLKSLFKQILSDRNAISWLPESFENDNQLLEKLESCYQSFNETYDDKKSIFVRFRELLLTISDYEMDKIFLRNDLQLTDISQKMFGSYSIISRSLLEDLKRGTSRKSKKETDESFEERLRNIIKNQDSFAIGTIDSSLQQMDVEEYKKSICDYFPNLSVDDKGDDIFDRIVKAYSEVKDLLNSPYPSDKNLAQEDDDIDKIKNLLESMKDLQKFVKPLCGKGNESDKDERFYGEFTALYEELDKITPLYNMVRNYLTRKPYSTEKIKLNFDNAQLLNGWDLNKESDNTSVILRKDGLYYLAIMNKKHNKVFEKNKLQSDGVCFEKMEYKLLPGANKMLPKVFFSKSRIDEFGPSQRLLDSYQNETHKKGDKFNIEDCHELIDFFKRSIDKHEDWSKFSFSFSDTKTYEDLSGFYREVEHQGYILSFVNVSVDYVNSLVDEGKIYLFQIYNKDFSPFSKGTPNMHTLYWKMLFDEENLKDVVYKLNGQAEVFFRKSSIKYDKPTHPANLPIDNKNVSNHKKRSVFEYDLVKDKRYTVDKFQFHVPVTINFKSDGNGNINPLVNDYIKKSDDLHVIGIDRGERHLLYLTVIDMKGNIKKQFSLNEIVNEYKGNTYSTNYHDLLEKREDKRDKERKEWKTIETIKELKEGYLSQVIHKITELMVEYNAIIVLEDLNLGFMRGRQKVEKSVYQKFEKMLIDKLNYLADKKKEPEDLGGVLKAYQLANKFESFQKMGKQSGFLFYTQAWNTSKIDPVTGFVNLFDTHYENILKSKNFFSKFDLIKYNSDKDWFEFSFDYNNFTTKAEGTKTKWTLCTFGNRIISFRNPDNNMQWDGKEINLTEEFKLFFEKFGININSDLHAEILKQDKKDFFEGLLHLLKLTLQMRNSKTRTDIDYMQSPVADENGVLYNSNKCGKSLPENADANGAYNIARKGLMIIDKIKKSDNLNKIDLTISNKEWLVFAQNKPYLKN

SEQ ID NO: 117 - protein, 1291 aa, synthetic construct
MGSGKAFENFTGLYPLSKTLRFELKPIGKTLEYIEKHGILDKDKHRANSYVKVKDIIDRYHKQFIEDSLSDSDFKLKYENKGKKESLEEYFYYYKLRNRDDKQKKDFDEIQKNLRKQIASQLKKQDRFKRIDKKELIKEDLLEFVSDDNERNLINEFKDFTTYFTGFHENRQNMYSDEAKSTAIAYRLIHENLPKFIDNISVFERVAATDVADCFAQIYSDFEEYLNVNDISEIFRLDYYTEILTQTQIDAYNLIIGGRSEGNIKIKGLNEYINLYNQQQKDKSQRLPKLKSLFKQILSDRNAISWLPESFENDNQLLEKLESCYQSFNETYDDKKSIFVRFRELLLTISDYEMDKIFLRNDLQLTDISQKMFGSYSIISRSLLEDLKRGTSRKSKKETDESFEERLRNIIKNQDSFAIGTIDSSLQQMDVEEYKKSICDYFPNLSVDDKGDDIFDRIVKAYSEVKDLLNSPYPSDKNLAQEDDDIDKIKNLLESMKDLQKFVKPLCGKGNESDKDERFYGEFTALYEELDKITPLYNMVRNYLTRKPYSTEKIKLNFDNAQLLNGWDLNKESDNTSVILRKDGLYYLAIMNKKHNKVFEKNKLQSDGVCFEKMEYKLLPGANKMLPKVFFSKSRIDEFGPSQRLLDSYQNETHKKGDKFNIEDCHELIDFFKRSIDKHEDWSKFSFSFSDTKTYEDLSGFYREVEHQGYILSFVNVSVDYVNSLVDEGKIYLFQIYNKDFSPFSKGTPNMHTLYWKMLFDEENLKDVVYKLNGQAEVFFRKSSIKYDKPTHPANLPIDNKNVSNHKKRSVFEYDLVKDKRYTVDKFQFHVPVTINFKSDGNGNINPLVNDYIKKSDDLHVIGIDRGERHLLYLTVIDMKGNIKKQFSLNEIVNEYKGNTYSTNYHDLLEKREDKRDKERKEWKTIETIKELKEGYLSQVIHKITELMVEYNAIIVLEDLNLGFMRGRQKVEKSVYQKFEKMLIDKLNYLADKKKEPEDLGGVLKAYQLANKFESFQKMGKQSGFLFYTQAWNTSKIDPVTGFVNLFDTHYENILKSKNFFSKFDLIKYNSDKDWFEFSFDYNNFTTKAEGTKTKWTLCTFGNRIISFRNPDNNMQWDGKEINLTEEFKLFFEKFGININSDLHAEILKQDKKDFFEGLLHLLKLTLQMRNSKTRTDIDYMQSPVADENGVLYNSNKCGKSLPENADANGAYNIARKGLMIIDKIKKSDNLNKIDLTISNKEWLVFAQNKPYLKNSRKRTADGSEFESPKKKRKVGSGKPIPNPLLGLDST

SEQ ID NO: 118 - DNA, 3759 bp, unidentified
  source 1..3759: mol_type = unassigned DNA
atgaaagcatttgagaattttacaggattgtatcctctttctaaaacattaagatttgagctgaaaccgattggaaagacattggaatatattgagaagcatggtattcttgataaggataaacacagagcaaatagttatgttaaggtcaaggatataattgacagatatcataaacaatttattgaagactcgttaagtgatagtgattttaaacttaaatatgaaaacaaaggaaagaaagaatcattagaagaatatttctattattataaattaagaaatagagacgacaaacagaagaaagattttgatgaaattcaaaagaatcttagaaaacagattgcaagtcaattaaagaaacaagatcgttttaaaagaattgataaaaaggaacttataaaggaagatcttttagaatttgttagtgatgataatgaaaggaatcttattaatgaatttaaagatttcacgacatattttacaggttttcacgaaaacagacaaaatatgtattctgatgaagccaaatcaactgcgatagcgtatagactgatacatgagaatcttcctaaatttatagataacatttcagtttttgaaagagttgctgctacagatgtggctgattgttttgcacaaatctattctgattttgaggaatatctgaatgtaaatgatatatctgaaatttttagattagactattatacggaaatattaactcagacacagattgatgcttataatctgataattggaggacgttctgagggcaatattaaaataaaaggtttgaacgaatatattaatctgtataatcaacagcagaaagacaagtctcaacggttgccaaaactgaagtctttgtttaaacagattttgagtgatagaaatgctatatcttggttgccagaatcgtttgaaaatgataatcaactcttggaaaagttggagagttgttatcagtcttttaatgaaacatatgacgataagaagtcaatatttgtaaggtttagagaattattgttgactatatctgattatgaaatggataaaatatttcttcgtaatgatttgcagttgacagatatttcacaaaagatgttcggtagttatagtattatttcaaggtctttattggaagatttaaagagaggtacatctcgtaaatcaaagaaggaaactgatgaaagttttgaagaaaggttgagaaatattatcaaaaaccaagatagttttgccattggaacaatagattcgtctttgcaacaaatggatgttgaagaatacaagaaatctatttgtgattatttccctaatttatctgttgatgacaaaggagatgatatttttgatagaatagtaaaagcgtattcggaggttaaagacttgttgaattctccgtatccgtcagataaaaaccttgctcaagaagatgatgatattgataagattaaaaatcttttagagtcaatgaaagatcttcagaagtttgtgaaacctctctgtggaaaaggaaatgaatctgataaagatgagcgtttctatggtgagtttacggctttatatgaagaattagacaagataacaccattatataatatggtgagaaattatcttactcgcaaaccgtattctacggaaaagataaagttaaactttgacaatgctcaacttttgaatggatgggatttaaataaagaaagtgataatacgagtgtcatattgcgtaaagacggattgtattatcttgccatcatgaacaagaagcataataaagtcttcgagaaaaataaattacagtcagatggtgtttgctttgaaaaaatggagtataaattacttcctggtgcaaacaagatgcttccaaaagttttcttctctaaatcaaggatagatgagtttggaccttctcaaagattgttggacagttatcagaatgaaactcataaaaaaggtgataaattcaatattgaagattgccatgaattgatagattttttcaaaaggtctattgataaacatgaggattggagtaaatttagctttagtttctcagatactaagacatatgaagatttaagcggattttacagagaagttgagcatcagggttatatactttcttttgtaaatgtttctgtagattatgtaaatagtttggtagatgaaggaaagatatatttatttcaaatttataataaagatttctcgccatttagcaaaggaactccaaatatgcatactttgtattggaaaatgctttttgatgaagaaaatctgaaagatgtggtgtataaattgaatggtcaggcagaagtgtttttcaggaaatccagtataaagtatgataaaccgactcatcctgctaatttgcctattgataataaaaatgtatctaaccataagaaacggagtgtctttgagtatgatttggtcaaagataagagatatacggttgataaattccagtttcatgttcctgtaacaatcaattttaaaagtgatggaaatggaaatatcaatcctctcgtcaatgattatatcaaaaagtctgatgatttgcatgtgattggtatcgacaggggagagcgtcatcttttgtatcttacggtcatagatatgaaaggtaatatcaagaagcagttttcattgaatgaaatcgtcaatgaatataaaggaaatacatatagtaccaattatcatgatttgttggaaaaacgcgaggacaaacgtgataaggaaagaaaagaatggaaaactatagaaaccatcaaggagttgaaagaaggttatctcagccaggttattcataaaataacggaattgatggttgaatataatgcaatcattgtgctggaggatcttaatttaggatttatgcgtgggcgacaaaaggtggagaagtctgtttatcaaaagtttgaaaagatgttgattgataaactgaattatcttgctgataaaaagaaagaaccggaagatttgggtggtgtgttgaaggcatatcaactggcaaataagtttgaaagttttcaaaaaatgggaaaacaatcaggtttcttattctatacccaagcatggaatacaagtaagatagatccggttactggttttgttaatctttttgacacacattatgagaatatcttaaagtctaaaaatttcttctctaagtttgatttgataaagtataattctgataaagattggttcgagttttcttttgattataataattttacaactaaagcagaaggtacaaaaacaaaatggacattatgtacctttggaaatagaataatatcattccgtaatcctgataataatatgcaatgggatggaaaagaaattaatcttactgaagaattcaagttattctttgagaaatttggaatcaatattaattctgatttgcatgcggaaatattaaaacaagataaaaaagacttctttgaaggtcttttgcatttgttgaaattgacattgcagatgcgtaatagtaagactcgcactgatatagattatatgcagtctcctgtagcagacgaaaacggagtgttatacaatagtaataaatgtggtaaatccttgccagaaaatgctgatgctaacggtgcgtataatattgcaagaaaaggtcttatgataattgacaaaataaagaagtctgataatctgaataaaatagatcttacgatctctaataaggagtggttggtattcgcacaaaataaaccatatttgaagaattga

SEQ ID NO: 119 - DNA, 3753 bp, synthetic construct
  source 1..3753: mol_type = other DNA
aaggccttcgagaacttcaccggcctgtatcccctctctaaaaccctgagatttgagctgaagccaatcggcaagaccctcgaatacattgagaagcacggcatcctggacaaggacaagcacagagccaatagctacgtgaaggtgaaggacatcatcgacagataccacaaacagttcatcgaggactctctgtctgatagcgacttcaagctaaagtacgagaacaaaggcaagaaggagagcctggaagagtacttctactactacaagctgcggaaccgggatgataagcaaaagaaagattttgatgagatccagaagaacctgagaaaacaaatcgccagccagctcaaaaaacaggacagattcaagcggatcgacaagaaagaactgatcaaggaagatctgctggagttcgtgagcgacgacaatgaaagaaacctgatcaacgagttcaaggattttactacatactttaccggcttccacgagaaccggcagaacatgtactctgatgaggccaagtccaccgccatcgcttatagactgattcacgagaatctgcctaagttcatcgataacataagcgtgttcgagcgggtcgcagctacagatgtggccgactgcttcgcccagatctactccgatttcgaggaatacctgaacgtgaacgacatcagcgagatcttcagactggactactatacagaaatcctgacccagacccagatcgacgcctacaatctgatcattggcggcagaagcgagggcaacatcaaaattaaaggcttgaacgagtacatcaatctgtacaaccagcagcagaaagacaagagccaaagactgcccaagctgaagagcctgtttaaacagatcctgagcgacagaaatgccatatcttggttgcctgagtctttcgagaacgataaccagctgctggagaagctggagagctgctaccagagcttcaacgaaacctacgacgacaagaagtctatctttgttagatttagagaactgctgctgacaatctctgactacgagatggacaaaatcttcctgagaaatgacctgcagctgaccgacatctcccaaaaaatgttcggatcttacagcatcatctcccggagcctgttagaggatctcaagagaggaaccagccggaagtcaaagaaggaaacagacgagagcttcgaagaacggctgcgcaacattatcaagaatcaggactcctttgccatcggcaccatcgatagcagcctgcagcagatggacgtggaagagtacaagaaatccatctgcgactatttccctaatctgagtgttgacgacaagggcgatgacatatttgacagaatcgtgaaagcctatagcgaggtgaaggacctgctgaactccccttaccctagcgacaagaacctggctcaggaggacgacgacatcgacaagatcaaaaacctgctggaaagcatgaaggacctgcagaagttcgtcaagcctctgtgtggcaagggcaacgagagcgataaggatgaaaggttctacggcgagttcacagccctgtacgaggaactggacaagatcacccctctgtacaatatggtgcggaactacctgacaagaaagccatactctaccgagaagatcaaactgaacttcgacaacgcccagctgctgaacggatgggacctgaataaagagagcgacaacaccagcgtcatcctgcgtaaggatggcctgtactacctggccatcatgaacaagaagcacaacaaggtgttcgagaagaacaagctccaaagcgatggcgtgtgcttcgagaagatggagtacaagctgctgcctggcgccaacaagatgctgccaaaggtgttcttctctaagagcagaatcgatgagttcggcccttctcagagactgctggacagctaccagaacgaaacccacaagaagggcgacaaattcaacatcgaggactgtcacgagctgatcgactttttcaaaagaagcatcgacaaacatgaagattggagcaagttttcttttagcttcagcgacaccaagacctacgaggacctgagcggcttctacagagaagtagaacaccagggctacatcctgagctttgtgaacgtgagcgtggattacgtgaacagcctggtggacgagggaaagatctacttatttcagatctacaacaaggatttcagccctttctctaagggcacccctaacatgcacacactgtactggaagatgctgttcgacgaggaaaacctgaaggatgtggtgtacaagctgaatggccaggccgaagtgttcttcagaaagtcctctatcaagtacgacaaacctacccatcctgccaatctccccatcgataacaagaacgtgagcaaccacaagaagcggagcgtgttcgagtacgacctggtgaaggacaaacgttacaccgtggataagttccagttccacgtgcccgtgaccatcaacttcaagagcgatggcaacggcaatatcaaccccctggtgaacgactacatcaagaagagcgacgatctacacgtgatcggcatcgacagaggagaacggcacctgctgtacctgacggtgatcgacatgaagggcaacatcaagaaacaatttagcctgaacgagatcgtgaacgaatataagggcaatacctacagcaccaactaccacgacctgctggagaaacgggaagataagagagataaggagagaaaggaatggaaaaccattgaaacaatcaaggaactgaaagaaggatatctgagccaggtgatccacaagatcaccgagctgatggtggagtacaacgccatcatcgtcctggaggacctgaacctgggcttcatgagagggagacagaaggtggagaagtccgtataccagaaatttgaaaagatgctgatcgacaagctgaactacctggctgacaagaaaaaggaacctgaggaccttggaggcgtcctgaaggcctaccagctggccaacaaattcgaatctttccaaaagatgggcaaacagagcggctttctgttttacacccaggcttggaacaccagcaagatcgaccccgtgacgggcttcgtgaacctcttcgatacacattacgagaacatcctgaagagcaagaatttcttcagcaagttcgatctcatcaaatataacagcgataaagattggttcgagttctcgttcgactacaacaatttcaccaccaaggccgagggcaccaaaacaaagtggacactgtgcaccttcggaaacagaatcatcagctttagaaaccctgacaacaacatgcagtgggatggcaaggagatcaacctgacagaggagttcaagctgttcttcgagaagttcggcatcaacatcaactccgacctgcacgctgagatcctgaagcaagacaagaaggacttcttcgagggcctgctgcacctgctgaaactgacactccagatgcggaacagcaagacgaggaccgatatcgactacatgcagagccccgtggccgacgagaatggggtgctgtacaactccaacaaatgcggcaagagcctgcccgagaacgccgatgccaacggagcctacaacatcgctagaaagggactgatgatcattgacaagatcaagaagtctgacaacctgaacaagatcgatctgactatctctaacaaggaatggctggtgttcgcccagaacaagccttacctgaaaaat

SEQ ID NO: 120 - DNA, 3876 bp, synthetic construct
  source 1..3876: mol_type = other DNA
atgggctccggaaaggccttcgagaacttcaccggcctgtatcccctctctaaaaccctgagatttgagctgaagccaatcggcaagaccctcgaatacattgagaagcacggcatcctggacaaggacaagcacagagccaatagctacgtgaaggtgaaggacatcatcgacagataccacaaacagttcatcgaggactctctgtctgatagcgacttcaagctaaagtacgagaacaaaggcaagaaggagagcctggaagagtacttctactactacaagctgcggaaccgggatgataagcaaaagaaagattttgatgagatccagaagaacctgagaaaacaaatcgccagccagctcaaaaaacaggacagattcaagcggatcgacaagaaagaactgatcaaggaagatctgctggagttcgtgagcgacgacaatgaaagaaacctgatcaacgagttcaaggattttactacatactttaccggcttccacgagaaccggcagaacatgtactctgatgaggccaagtccaccgccatcgcttatagactgattcacgagaatctgcctaagttcatcgataacataagcgtgttcgagcgggtcgcagctacagatgtggccgactgcttcgcccagatctactccgatttcgaggaatacctgaacgtgaacgacatcagcgagatcttcagactggactactatacagaaatcctgacccagacccagatcgacgcctacaatctgatcattggcggcagaagcgagggcaacatcaaaattaaaggcttgaacgagtacatcaatctgtacaaccagcagcagaaagacaagagccaaagactgcccaagctgaagagcctgtttaaacagatcctgagcgacagaaatgccatatcttggttgcctgagtctttcgagaacgataaccagctgctggagaagctggagagctgctaccagagcttcaacgaaacctacgacgacaagaagtctatctttgttagatttagagaactgctgctgacaatctctgactacgagatggacaaaatcttcctgagaaatgacctgcagctgaccgacatctcccaaaaaatgttcggatcttacagcatcatctcccggagcctgttagaggatctcaagagaggaaccagccggaagtcaaagaaggaaacagacgagagcttcgaagaacggctgcgcaacattatcaagaatcaggactcctttgccatcggcaccatcgatagcagcctgcagcagatggacgtggaagagtacaagaaatccatctgcgactatttccctaatctgagtgttgacgacaagggcgatgacatatttgacagaatcgtgaaagcctatagcgaggtgaaggacctgctgaactccccttaccctagcgacaagaacctggctcaggaggacgacgacatcgacaagatcaaaaacctgctggaaagcatgaaggacctgcagaagttcgtcaagcctctgtgtggcaagggcaacgagagcgataaggatgaaaggttctacggcgagttcacagccctgtacgaggaactggacaagatcacccctctgtacaatatggtgcggaactacctgacaagaaagccatactctaccgagaagatcaaactgaacttcgacaacgcccagctgctgaacggatgggacctgaataaagagagcgacaacaccagcgtcatcctgcgtaaggatggcctgtactacctggccatcatgaacaagaagcacaacaaggtgttcgagaagaacaagctccaaagcgatggcgtgtgcttcgagaagatggagtacaagctgctgcctggcgccaacaagatgctgccaaaggtgttcttctctaagagcagaatcgatgagttcggcccttctcagagactgctggacagctaccagaacgaaacccacaagaagggcgacaaattcaacatcgaggactgtcacgagctgatcgactttttcaaaagaagcatcgacaaacatgaagattggagcaagttttcttttagcttcagcgacaccaagacctacgaggacctgagcggcttctacagagaagtagaacaccagggctacatcctgagctttgtgaacgtgagcgtggattacgtgaacagcctggtggacgagggaaagatctacttatttcagatctacaacaaggatttcagccctttctctaagggcacccctaacatgcacacactgtactggaagatgctgttcgacgaggaaaacctgaaggatgtggtgtacaagctgaatggccaggccgaagtgttcttcagaaagtcctctatcaagtacgacaaacctacccatcctgccaatctccccatcgataacaagaacgtgagcaaccacaagaagcggagcgtgttcgagtacgacctggtgaaggacaaacgttacaccgtggataagttccagttccacgtgcccgtgaccatcaacttcaagagcgatggcaacggcaatatcaaccccctggtgaacgactacatcaagaagagcgacgatctacacgtgatcggcatcgacagaggagaacggcacctgctgtacctgacggtgatcgacatgaagggcaacatcaagaaacaatttagcctgaacgagatcgtgaacgaatataagggcaatacctacagcaccaactaccacgacctgctggagaaacgggaagataagagagataaggagagaaaggaatggaaaaccattgaaacaatcaaggaactgaaagaaggatatctgagccaggtgatccacaagatcaccgagctgatggtggagtacaacgccatcatcgtcctggaggacctgaacctgggcttcatgagagggagacagaaggtggagaagtccgtataccagaaatttgaaaagatgctgatcgacaagctgaactacctggctgacaagaaaaaggaacctgaggaccttggaggcgtcctgaaggcctaccagctggccaacaaattcgaatctttccaaaagatgggcaaacagagcggctttctgttttacacccaggcttggaacaccagcaagatcgaccccgtgacgggcttcgtgaacctcttcgatacacattacgagaacatcctgaagagcaagaatttcttcagcaagttcgatctcatcaaatataacagcgataaagattggttcgagttctcgttcgactacaacaatttcaccaccaaggccgagggcaccaaaacaaagtggacactgtgcaccttcggaaacagaatcatcagctttagaaaccctgacaacaacatgcagtgggatggcaaggagatcaacctgacagaggagttcaagctgttcttcgagaagttcggcatcaacatcaactccgacctgcacgctgagatcctgaagcaagacaagaaggacttcttcgagggcctgctgcacctgctgaaactgacactccagatgcggaacagcaagacgaggaccgatatcgactacatgcagagccccgtggccgacgagaatggggtgctgtacaactccaacaaatgcggcaagagcctgcccgagaacgccgatgccaacggagcctacaacatcgctagaaagggactgatgatcattgacaagatcaagaagtctgacaacctgaacaagatcgatctgactatctctaacaaggaatggctggtgttcgcccagaacaagccttacctgaaaaattctagaaagcggacagcagacggctccgaatttgaaagccctaagaaaaagagaaaggtgggatccggcaaacctatccccaatcccctgctgggcctggacagcacctga

SEQ ID NO: 121 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 122 - protein, 18 aa, synthetic construct
KRTADGSEFESPKKKRKV

SEQ ID NO: 123 - protein, 7 aa, synthetic construct
PKKKRKV

SEQ ID NO: 124 - protein, 7 aa, synthetic construct
PKKKRRV

SEQ ID NO: 125 - protein, 16 aa, synthetic construct
KRPAATKKAGQAKKKK

SEQ ID NO: 126 - protein, 11 aa, synthetic construct
YGRKKRRQRRR

SEQ ID NO: 127 - protein, 9 aa, synthetic construct
RKKRRQRRR

SEQ ID NO: 128 - protein, 9 aa, synthetic construct
PAAKRVKLD

SEQ ID NO: 129 - protein, 11 aa, synthetic construct
RQRRNELKRSP

SEQ ID NO: 130 - protein, 8 aa, synthetic construct
VSRKRPRP

SEQ ID NO: 131 - protein, 8 aa, synthetic construct
PPKKARED

SEQ ID NO: 132 - protein, 8 aa, synthetic construct
PQPKKKPL

SEQ ID NO: 133 - protein, 12 aa, synthetic construct
SALIKKKKKMAP

SEQ ID NO: 134 - protein, 7 aa, synthetic construct
PKQKKRK

SEQ ID NO: 135 - protein, 10 aa, synthetic construct
RKLKKKIKKL

SEQ ID NO: 136 - protein, 10 aa, synthetic construct
REKKKFLKRR

SEQ ID NO: 137 - protein, 20 aa, synthetic construct
KRKGDEVDGVDEVAKKKSKK

SEQ ID NO: 138 - protein, 17 aa, synthetic construct
RKCLQAGMNLEARKTKK

SEQ ID NO: 139 - protein, 38 aa, synthetic construct
NQSSNFGPMKGGNFGGRSSGPYGGGGQYFAKPRNQGGY

SEQ ID NO: 140 - protein, 42 aa, synthetic construct
RMRIZFKNKGKDTAELRRRRVEVSVELRKAKKDEQILKRRNV

SEQ ID NO: 141 - protein, 14 aa, synthetic construct
GKPIPNPLLGLDST

SEQ ID NO: 142 - protein, 9 aa, synthetic construct
IPNPLLGLD

SEQ ID NO: 143 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 144 - RNA, 31 nt, synthetic construct
  source 1..31: mol_type = other RNA
acgattagaaataatttctactgttgtagat

SEQ ID NO: 145 - RNA, 36 nt, synthetic construct
  source 1..36: mol_type = other RNA
ctttgaaagaatataatttctactgaaagtgtagat

SEQ ID NO: 146 - RNA, 36 nt, synthetic construct
  source 1..36: mol_type = other RNA
gtttaataataatacataatttctactattgtagat

SEQ ID NO: 147 - RNA, 36 nt, synthetic construct
  source 1..36: mol_type = other RNA
gtctataagactaatttaatttctactattgtagat

SEQ ID NO: 148 - RNA, 36 nt, synthetic construct
  source 1..36: mol_type = other RNA
gtttataaaccttatctaatttctactgttgtagat

SEQ ID NO: 149 - RNA, 35 nt, synthetic construct
  source 1..35: mol_type = other RNA
tctaaagctctttaagaatttctactttcgtagat

SEQ ID NO: 150 - RNA, 34 nt, synthetic construct
  source 1..34: mol_type = other RNA
ctaagaggctcaaataatttctactattgtagat

SEQ ID NO: 151 - RNA, 36 nt, synthetic construct
  source 1..36: mol_type = other RNA
cctttggaagtactaagaatttctactgttgtagat

SEQ ID NO: 152 - RNA, 36 nt, synthetic construct
  source 1..36: mol_type = other RNA
ggctataaaagccatataatttctactattgtagat

SEQ ID NO: 153 - RNA, 36 nt, synthetic construct
  source 1..36: mol_type = other RNA
gactattaagtcttttgaatttctactgttgtagat

SEQ ID NO: 154 - RNA, 35 nt, synthetic construct
  source 1..35: mol_type = other RNA
tctagaatatataggtaatttctacttatgtagat

SEQ ID NO: 155 - RNA, 37 nt, synthetic construct
  source 1..37: mol_type = other RNA
ggcaataagccatatacaatttctactgtatgtagat

SEQ ID NO: 156 - RNA, 36 nt, synthetic construct
  source 1..36: mol_type = other RNA
gtctgaaagactatataatttctacttcgtgtagat

SEQ ID NO: 157 - RNA, 36 nt, synthetic construct
  source 1..36: mol_type = other RNA
gtctacggaacgtctgtaatttctactgttgtagat

SEQ ID NO: 158 - RNA, 35 nt, synthetic construct
  source 1..35: mol_type = other RNA
tttaaacgaactattaaatttctactgttgtagat

SEQ ID NO: 159 - RNA, 36 nt, synthetic construct
  source 1..36: mol_type = other RNA
gtttaatacttatatataatttctactattgtagat

SEQ ID NO: 160 - RNA, 35 nt, synthetic construct
  source 1..35: mol_type = other RNA
aataataatcccttataatttctacttttgtagat

SEQ ID NO: 161 - RNA, 36 nt, synthetic construct
  source 1..36: mol_type = other RNA
gtctataagacgaactaaatttctactattgtagat

SEQ ID NO: 162 - RNA, 36 nt, synthetic construct
  source 1..36: mol_type = other RNA
ggctactaagcctttataatttctactattgtagat

SEQ ID NO: 163 - RNA, 36 nt, synthetic construct
  source 1..36: mol_type = other RNA
gtctataaagacgaatgaatttctactattgtagat

SEQ ID NO: 164 - RNA, 23 nt, synthetic construct
  source 1..23: mol_type = other RNA
tggcctggaggctatccagcgtg

SEQ ID NO: 165 - RNA, 23 nt, synthetic construct
  source 1..23: mol_type = other RNA
ctcacgtcatccagcagagaatg

SEQ ID NO: 166 - RNA, 23 nt, synthetic construct
  source 1..23: mol_type = other RNA
actttccattctctgctggatga

SEQ ID NO: 167 - RNA, 23 nt, synthetic construct
  source 1..23: mol_type = other RNA
ctgaattgctatgtgtctgggtt

SEQ ID NO: 168 - RNA, 23 nt, synthetic construct
  source 1..23: mol_type = other RNA
aattctctctccattcttcagta

SEQ ID NO: 169 - RNA, 23 nt, synthetic construct
  source 1..23: mol_type = other RNA
agaatcaaaatcggtgaataggc

SEQ ID NO: 170 - RNA, 23 nt, synthetic construct
  source 1..23: mol_type = other RNA
tgacacatttgtttgagaatcaa

SEQ ID NO: 171 - RNA, 23 nt, synthetic construct
  source 1..23: mol_type = other RNA
gagtctctcagctggtacacggc

SEQ ID NO: 172 - RNA, 23 nt, synthetic construct
  source 1..23: mol_type = other RNA
tctgtgatatacacatcagaatc

SEQ ID NO: 173 - RNA, 23 nt, synthetic construct
  source 1..23: mol_type = other RNA
attctcaaacaaatgtgtcacaa

SEQ ID NO: 174 - RNA, 23 nt, synthetic construct
  source 1..23: mol_type = other RNA
ccttccgctcacctccgcctgag

SEQ ID NO: 175 - RNA, 23 nt, synthetic construct
  source 1..23: mol_type = other RNA
gcacgaagctctccgatgtgttg

SEQ ID NO: 176 - RNA, 23 nt, synthetic construct
  source 1..23: mol_type = other RNA
atctgcgccttgggggccaggga

SEQ ID NO: 177 - RNA, 23 nt, synthetic construct
  source 1..23: mol_type = other RNA
gaactggccggctggcctgggtg

SEQ ID NO: 178 - protein, 22 aa, synthetic construct
SSDDEATADSQHAAPPKKKRKV

SEQ ID NO: 179 - protein, 8 aa, synthetic construct
PKKKRKVG

SEQ ID NO: 180 - protein, 24 aa, synthetic construct
GRSSDDEATADSQHAAPPKKKRKV

SEQ ID NO: 181 - RNA, 23 nt, synthetic construct
  source 1..23: mol_type = other RNA
gtgtcaagctatatcaggcacca

SEQ ID NO: 182 - RNA, 23 nt, synthetic construct
  source 1..23: mol_type = other RNA
atgtgtcttttcccgatattcct

SEQ ID NO: 183 - RNA, 21 nt, synthetic construct
  source 1..21: mol_type = other RNA
tggcctggaggctatccagcg

SEQ ID NO: 184 - RNA, 23 nt, synthetic construct
  source 1..23: mol_type = other RNA
tcactggatttagagtctctcag

SEQ ID NO: 185 - RNA, 23 nt, synthetic construct
  source 1..23: mol_type = other RNA
gattctcaaacaaatgtgtcaca

SEQ ID NO: 186 - RNA, 23 nt, synthetic construct
  source 1..23: mol_type = other RNA
aagagggaaatgagatcatgtcc

SEQ ID NO: 187 - RNA, 23 nt, synthetic construct
  source 1..23: mol_type = other RNA
accgattttgattctcaaacaaa

SEQ ID NO: 188 - RNA, 23 nt, synthetic construct
  source 1..23: mol_type = other RNA
gtctgtgatatacacatcagaat

SEQ ID NO: 189 - RNA, 20 nt, synthetic construct
  source 1..20: mol_type = other RNA
gagtctctcagctggtacac

SEQ ID NO: 190 - RNA, 21 nt, synthetic construct
  source 1..21: mol_type = other RNA
gagtctctcagctggtacacg

SEQ ID NO: 191 - RNA, 22 nt, synthetic construct
  source 1..22: mol_type = other RNA
gagtctctcagctggtacacgg

SEQ ID NO: 192 - RNA, 24 nt, synthetic construct
  source 1..24: mol_type = other RNA
gagtctctcagctggtacacggca

SEQ ID NO: 193 - RNA, 23 nt, synthetic construct
  source 1..23: mol_type = other RNA
atttgggcagctcccctaccccc

SEQ ID NO: 194 - RNA, 23 nt, synthetic construct
  source 1..23: mol_type = other RNA
ggcagctcccctaccccccttac

SEQ ID NO: 195 - RNA, 23 nt, synthetic construct
  source 1..23: mol_type = other RNA
caggggtccgagagctcagctag

SEQ ID NO: 196 - RNA, 23 nt, synthetic construct
  source 1..23: mol_type = other RNA
atctgtcccctccaccccacagt

SEQ ID NO: 197 - RNA, 23 nt, synthetic construct
  source 1..23: mol_type = other RNA
tactttgtcctccggttctggaa

SEQ ID NO: 198 - RNA, 23 nt, synthetic construct
  source 1..23: mol_type = other RNA
tcctccggttctggaaccacacc

SEQ ID NO: 199 - RNA, 23 nt, synthetic construct
  source 1..23: mol_type = other RNA
agccatctcactacagataactc

SEQ ID NO: 200 - RNA, 23 nt, synthetic construct
  source 1..23: mol_type = other RNA
aagctagtctagtgcaagctaac

SEQ ID NO: 201 - RNA, 23 nt, synthetic construct
  source 1..23: mol_type = other RNA
gcctctgattagggtgggggcgt

SEQ ID NO: 202 - RNA, 21 nt, synthetic construct
  source 1..21: mol_type = other RNA
tcacaggctccaggaagggtt

SEQ ID NO: 203 - RNA, 23 nt, synthetic construct
  source 1..23: mol_type = other RNA
tctgccacccacctcctcacctt

SEQ ID NO: 204 - RNA, 23 nt, synthetic construct
  source 1..23: mol_type = other RNA
caggtcatcacagttggggccac

SEQ ID NO: 205 - RNA, 21 nt, synthetic construct
  source 1..21: mol_type = other RNA
gagagtgaggacgtgtgtgtc

SEQ ID NO: 206 - RNA, 20 nt, synthetic construct
  source 1..20: mol_type = other RNA
gggtgatcagacccaacagc

SEQ ID NO: 207 - RNA, 21 nt, synthetic construct
  source 1..21: mol_type = other RNA
gggtgatcagacccaacagca

SEQ ID NO: 208 - RNA, 22 nt, synthetic construct
  source 1..22: mol_type = other RNA
gggtgatcagacccaacagcag

SEQ ID NO: 209 - RNA, 23 nt, synthetic construct
  source 1..23: mol_type = other RNA
gggtgatcagacccaacagcagg

SEQ ID NO: 210 - RNA, 24 nt, synthetic construct
  source 1..24: mol_type = other RNA
gggtgatcagacccaacagcaggt

SEQ ID NO: 211 - RNA, 20 nt, synthetic construct
  source 1..20: mol_type = other RNA
gaatttctactgttgtagat

SEQ ID NO: 212 - RNA, 20 nt, synthetic construct
  source 1..20: mol_type = other RNA
taatttctactattgtagat

SEQ ID NO: 213 - RNA, 20 nt, synthetic construct
  source 1..20: mol_type = other RNA
aatttctacttcgtgtagat

SEQ ID NO: 214 - protein, 228 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 215 - protein, 228 aa, synthetic construct
SSKTGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPNVTLFIYIARLYHLANPRNRQGLRDLISSGVTIQIMTEQESGYCWHNFVNYSPSNESHWPRYPHLWVRLYVLELYCIILGLPPCLNILRRKQSQLTSFTIALQSCHYQRLPPHILWATGLK

SEQ ID NO: 216 - protein, 228 aa, synthetic construct
SSETGPVAVDPTLRRRIEPHEFEVFFDPRELRKETCLLYEINWGGRHSIWRHTSQNTNKHVEVNFIEKFTTERYFCPNTRCSITWFLSYSPCGECSRAITEFLSRYPHVTLFIYIARLYHHADPENRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLK

SEQ ID NO: 217 - protein, 162 aa, synthetic construct
SFERNYDPRELRKETYLLYEIKWGKSGKLWRHWCQNNRTQHAEVYFLENIFNARRFNPSTHCSITWYLSWSPCAECSQKIVDFLKEHPNVNLEIYVARLYYHEDERNRQGLRDLVNSGVTIRIMDLPDYNYCWKTFVSDQGGDEDYWPGHFAPWIKQYSLKL

SEQ ID NO: 218 - protein, 237 aa, synthetic construct
TSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWRLK

SEQ ID NO: 219 - protein, 191 aa, synthetic construct
ADSSEKMRGQYISRDTFEKNYKPIDGTKEAHLLCEIKWGKYGKPWLHWCQNQRMNIHAEDYFMNNIFKAKKHPVHCYVTWYLSWSPCADCASKIVKFLEERPYLKLTIYVAQLYYHTEEENRKGLRLLRSKKVIIRVMDISDYNYCWKVFVSNQNGNEDYWPLQFDPWVKENYSRLLDIFWESKCRSPNPW

SEQ ID NO: 220 - protein, 228 aa, synthetic construct
SSETGPVAVDPTLRRRIEPHEFEVFFDPRELRKETCLLYEIKWGTSHKIWRHSSKNTTKHVEVNFIEKFTSERHFCPSTSCSITWFLSWSPCGECSKAITEFLSQHPNVTLVIYVARLYHHMDQQNRQGLRDLVNSGVTIQIMTAPEYDYCWRNFVNYPPGKEAHWPRYPPLWMKLYALELHAGILGLPPCLNILRRKQPQLTFFTIALQSCHYQRLPPHILWATGLK

SEQ ID NO: 221 - protein, 198 aa, synthetic construct
EASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 222 - protein, 198 aa, synthetic construct
EASPASGPRHLMDPHIFTSNFNNGIGRHKTYLCYEVERLDNGTSVKMDQHRGFLHGQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 223 - protein, 381 aa, synthetic construct
NPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 224 - protein, 189 aa, synthetic construct
NPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 225 - protein, 385 aa, synthetic construct
NPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 226 - protein, 372 aa, synthetic construct
KPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 227 - protein, 383 aa, synthetic construct
KPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 228 - protein, 182 aa, synthetic construct
ALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKQS

SEQ ID NO: 229 - protein, 193 aa, synthetic construct
KPQIRDHRPNPMEAMYPHIFYFHFENLEKAYGRNETWLCFTVEIIKQYLPVPWKKGVFRNQVDPETHCHAEKCFLSWFCNNTLSPKKNYQVTWYTSWSPCPECAGEVAEFLAEHSNVKLTIYTARLYYFWDTDYQEGLRSLSEEGASVEIMDYEDFQYCWENFVYDDGEPFKRWKGLKYNFQSLTRRLREILQ

SEQ ID NO: 230 - protein, 279 aa, synthetic construct
DPQRLRQWPGPGPASRGGYGQRPRIRNPEEWFHELSPRTFSFHFRNLRFASGRNRSYICCQVEGKNCFFQGIFQNQVPPDPPCHAELCFLSWFQSWGLSPDEHYYVTWFISWSPCCECAAKVAQFLEENRNVSLSLSAARLYYFWKSESREGLRRLSDLGAQVGIMSFQDFQHCWNNFVHNLGMPFQPWKKLHKNYQRLVTELKQILREEPATYGSPQAQGKVRIGSTAAGLRHSHSHTRSEAHLRPNHSSRQHRILNPPREARARTCVLVDASWICYR

SEQ ID NO: 231 - protein, 197 aa, synthetic construct
DSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGL

SEQ ID NO: 232 - protein, 175 aa, synthetic construct
DPATFTYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLAEAGVQIAIMTFKDYFYCWNTFVENHERTFKAWEGLHENSVRLSRQLRRILQ

SEQ ID NO: 233 - protein, 174 aa, synthetic construct
DPATFTYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYYFQYPCYQEGLRRLHRAGVQIAIMTFKDYFYCWNTFVENHERTFKAWEGLHENSVRLSRQLRRILQ

SEQ ID NO: 234 - protein, 174 aa, synthetic construct
DPATFTYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYYFWDTDYQEGLRRLHRAGVQIAIMTFKDYFYCWNTFVENHERTFKAWEGLHENSVRLSRQLRRILQ

SEQ ID NO: 235 - protein, 207 aa, synthetic construct
TDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 236 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 237 - protein, 166 aa, synthetic construct; identical to SEQ ID NO: 143

SEQ ID NO: 238 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 239 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRAWDEREVPVGAVLVLNNRVIGEGWNRSIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMRRQVFNAQKKAQSSTD

SEQ ID NO: 240 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMRRQVFNAQKKAQSSTD

SEQ ID NO: 241 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRALDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECNALLCYFFRMPRQVFNAQKKAQSSTD

SEQ ID NO: 242 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 243 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 244 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 245 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 246 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTD

SEQ ID NO: 247 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYSTFEPCVMCAGAMIHSRIGRVVFGVRNAKTGAAGSLMDVLHYPGMNHRVEITEGILADECAALLCYFFRMPRRVFNAQKKAQSSTD

SEQ ID NO: 248 - protein, 166 aa, synthetic construct
SEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 249 - protein, 364 aa, synthetic construct
SEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLYDATLYSTFEPCVMCAGAMIHSRIGRVVFGVRNAKTGAAGSLMDVLHHPGMNHRVEITEGILADECAALLCRFFRMPRRVFNAQKKAQSSTD

SEQ ID NO: 250 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 251 - protein, 117 aa, synthetic construct
ALSPQHSAVTQGSIIKNKEGMDAKSLTAWSRTLVTFKDVFVDFTREEWKLLDTAQQIVYRNVMLENYKNLVSLGYQLTKPDVILRLEKGEEPWLVEREIHQETHPDSETAFEIKSSV

SEQ ID NO: 252 - protein, 66 aa, synthetic construct
SRTLVTFKDVFVDFTREEWKLLDTAQQIVYRNVMLENYKNLVSLGYQLTKPDVILRLEKGEEPWLV

SEQ ID NO: 253 - protein, 323 aa, synthetic construct
GTYGLLRRREDWPSRLQMFFANNHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACV

SEQ ID NO: 254 - protein, 385 aa, synthetic construct
AAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 255 - protein, 714 aa, synthetic construct
NHDQEFDPPKVYPPVPAEKRKPIRVLSLFDGIATGLLVLKDLGIQVDRYIASEVCEDSITVGMVRHQGKIMYVGDVRSVTQKHIQEWGPFDLVIGGSPCNDLSIVNPARKGLYEGTGRLFFEFYRLLHDARPKEGDDRPFFWLFENVVAMGVSDKRDISRFLESNPVMIDAKEVSAAHRARYFWGNLPGMNRPLASTVNDKLELQECLEHGRIAKFSKVRTITTRSNSIKQGKDQHFPVFMNEKEDILWCTEMERVFGFPVHYTDVSNMSRLARQRLLGRSWSVPVIRHLFAPLKEYFACVSSGNSNANSRGPSFSSGLVPLSLRGSHMAAIPALDPEAEPSMDVILVGSSELSSSVSPGTGRDLIAYEVKANQRNIEDICICCGSLQVHTQHPLFEGGICAPCKDKFLDALFLYDDDGYQSYCSICCSGETLLICGNPDCTRCYCFECVDSLVGPGTSGKVHAMSNWVCYLCLPSSRSGLLQRRRKWRSQLKAFYDRESENPLEMFETVPVWRRQPVRVLSLFEDIKKELTSLGFLESGSDPGQLKHVVDVTDTVRKDVEEWGPFDLVYGATPPLGHTCDRPPSWYLFQFHRLLQYARPKPGSPRPFFWMFVDNLVLNKEDLDVASRFLEMEPVTIPDVHGGSLQNAVRVWSNIPAIRSRHWALVSEEELSLLAQNKQSSKLAAKWPTKLVKNCFLPLREYFKYFSTELTSSL

SEQ ID NO: 256 - protein, 677 aa, synthetic construct
TLNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGWLTSEGKEIKNKDEILALLKALFLPKRLSIIHCPGHQKGHSAEARGNRMADQAARKAAITETPDTSTLLIENSSP

SEQ ID NO: 257 - protein, 506 aa, synthetic construct
ISSSKHTLSQMNKVSNIVKEPELPDIYKEFKDITADTNTEKLPKPIKGLEFEVELTQENYRLPIRNYPLTPVKMQAMNDEINQGLKGGIIRESKAINACPVIFVPRKEGTLRMVVDYRPLNKYVKPNVYPLPLIEQLLAKIQGSTIFTKLDLKSAYHQIRVRKGDEHKLAFRCPRGVFEYLVMPYGIKTAPAHFQYFINTILGEAKESHVVCYMDDILIHSKSESEHVKHVKDVLQKLKNANLIINQAKCEFHQSQVKFLGYHISEKGLTPCQENIDKVLQWKQPKNQKELRQFLGQVNYLRKFIPKTSQLTHPLNKLLKKDVRWKWTPTQTQAIENIKQCLVSPPVLRHFDFSKKILLETDVSDVAVGAVLSQKHDDDKYYPVGYYSAKMSKAQLNYSVSDKEMLAIIKSLEHWRHYLESTIEPFKILTDHRNLIGRITNESEPENKRLARWQLFLQDFNFEINYRPGSANHIADALSRIVDETEPIPKDNEDNSINFVNQISIS

SEQ ID NO: 258 - protein, 752 aa, synthetic construct
SFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVF

SEQ ID NO: 259 - protein, 4 aa, synthetic construct
GSGS

SEQ ID NO: 260 - RNA, 23 nt, synthetic construct
  source 1..23: mol_type = other RNA
cgtcgccgtccagctcgaccagg

SEQ ID NO: 261 - RNA, 23 nt, synthetic construct
  source 1..23: mol_type = other RNA
ctcagggcggactgggtgctcag

SEQ ID NO: 262 - DNA, 27 bp, synthetic construct
  source 1..27: mol_type = other DNA
agatcgtcgccgtccagctcgaccagg

SEQ ID NO: 263 - DNA, 27 bp, synthetic construct
  source 1..27: mol_type = other DNA
agatctcagggcggactgggtgctcag

SEQ ID NO: 264 - DNA, 27 bp, synthetic construct
  source 1..27: mol_type = other DNA
agattggcctggaggctatccagcgtg

SEQ ID NO: 265 - DNA, 27 bp, synthetic construct
  source 1..27: mol_type = other DNA
agatctcacgtcatccagcagagaatg

SEQ ID NO: 266 - DNA, 27 bp, synthetic construct
  source 1..27: mol_type = other DNA
agatactttccattctctgctggatga

SEQ ID NO: 267 - DNA, 27 bp, synthetic construct
  source 1..27: mol_type = other DNA
agatctgaattgctatgtgtctgggtt

SEQ ID NO: 268 - DNA, 27 bp, synthetic construct
  source 1..27: mol_type = other DNA
agataattctctctccattcttcagta

SEQ ID NO: 269 - DNA, 27 bp, synthetic construct
  source 1..27: mol_type = other DNA
agatagaatcaaaatcggtgaataggc

SEQ ID NO: 270 - DNA, 27 bp, synthetic construct
  source 1..27: mol_type = other DNA
agattgacacatttgtttgagaatcaa

SEQ ID NO: 271 - DNA, 27 bp, synthetic construct
  source 1..27: mol_type = other DNA
agatgagtctctcagctggtacacggc

SEQ ID NO: 272 - DNA, 27 bp, synthetic construct
  source 1..27: mol_type = other DNA
agattctgtgatatacacatcagaatc

SEQ ID NO: 273 - DNA, 27 bp, synthetic construct
  source 1..27: mol_type = other DNA
agatattctcaaacaaatgtgtcacaa

SEQ ID NO: 274 - DNA, 27 bp, synthetic construct
  source 1..27: mol_type = other DNA
agatccttccgctcacctccgcctgag

SEQ ID NO: 275 - DNA, 27 bp, synthetic construct
  source 1..27: mol_type = other DNA
agatgcacgaagctctccgatgtgttg

SEQ ID NO: 276 - DNA, 27 bp, synthetic construct
  source 1..27: mol_type = other DNA
agatatctgcgccttgggggccaggga

SEQ ID NO: 277 - DNA, 27 bp, synthetic construct
  source 1..27: mol_type = other DNA
agatgaactggccggctggcctgggtg

SEQ ID NO: 278 - DNA, 27 bp, synthetic construct
  source 1..27: mol_type = other DNA
agatcaggggtccgagagctcagctag

SEQ ID NO: 279 - DNA, 27 bp, synthetic construct
  source 1..27: mol_type = other DNA
agatatctgtcccctccaccccacagt

SEQ ID NO: 280 - DNA, 27 bp, synthetic construct
  source 1..27: mol_type = other DNA
agatggcagctcccctaccccccttac

SEQ ID NO: 281 - DNA, 27 bp, synthetic construct
  source 1..27: mol_type = other DNA
agatgtgtcaagctatatcaggcacca

SEQ ID NO: 282 - DNA, 27 bp, synthetic construct
  source 1..27: mol_type = other DNA
agatatgtgtcttttcccgatattcct

SEQ ID NO: 283 - DNA, 27 bp, synthetic construct
  source 1..27: mol_type = other DNA
agattcactggatttagagtctctcag

SEQ ID NO: 284 - DNA, 27 bp, synthetic construct
  source 1..27: mol_type = other DNA
agatgattctcaaacaaatgtgtcaca

SEQ ID NO: 285 - DNA, 27 bp, synthetic construct
  source 1..27: mol_type = other DNA
agataagagggaaatgagatcatgtcc

SEQ ID NO: 286 - DNA, 27 bp, synthetic construct
  source 1..27: mol_type = other DNA
agataccgattttgattctcaaacaaa

SEQ ID NO: 287 - DNA, 27 bp, synthetic construct
  source 1..27: mol_type = other DNA
agatgtctgtgatatacacatcagaat

SEQ ID NO: 288 - DNA, 27 bp, synthetic construct
  source 1..27: mol_type = other DNA
agatagccatctcactacagataactc

SEQ ID NO: 289 - DNA, 27 bp, synthetic construct
  source 1..27: mol_type = other DNA
agatatttgggcagctcccctaccccc

SEQ ID NO: 290 - DNA, 27 bp, synthetic construct
  source 1..27: mol_type = other DNA
agattactttgtcctccggttctggaa

SEQ ID NO: 291 - DNA, 27 bp, synthetic construct
  source 1..27: mol_type = other DNA
agattcctccggttctggaaccacacc

SEQ ID NO: 292 - DNA, 27 bp, synthetic construct
  source 1..27: mol_type = other DNA
agataagctagtctagtgcaagctaac

SEQ ID NO: 293 - DNA, 27 bp, synthetic construct
  source 1..27: mol_type = other DNA
agatgcctctgattagggtgggggcgt

SEQ ID NO: 294 - DNA, 27 bp, synthetic construct
  source 1..27: mol_type = other DNA
agattctgccacccacctcctcacctt

SEQ ID NO: 295 - DNA, 27 bp, synthetic construct
  source 1..27: mol_type = other DNA
agatcaggtcatcacagttggggccac

SEQ ID NO: 296 - DNA, 25 bp, synthetic construct
  source 1..25: mol_type = other DNA
agattcacaggctccaggaagggtt

SEQ ID NO: 297 - DNA, 25 bp, synthetic construct
  source 1..25: mol_type = other DNA
agatgagagtgaggacgtgtgtgtc

SEQ ID NO: 298 - DNA, 25 bp, synthetic construct
  source 1..25: mol_type = other DNA
agattggcctggaggctatccagcg

SEQ ID NO: 299 - DNA, 24 bp, synthetic construct
  source 1..24: mol_type = other DNA
agatgagtctctcagctggtacac

SEQ ID NO: 300 - DNA, 25 bp, synthetic construct
  source 1..25: mol_type = other DNA
agatgagtctctcagctggtacacg

SEQ ID NO: 301 - DNA, 26 bp, synthetic construct
  source 1..26: mol_type = other DNA
agatgagtctctcagctggtacacgg

SEQ ID NO: 302 - DNA, 28 bp, synthetic construct
  source 1..28: mol_type = other DNA
agatgagtctctcagctggtacacggca

SEQ ID NO: 303 - DNA, 24 bp, synthetic construct
  source 1..24: mol_type = other DNA
agatgggtgatcagacccaacagc

SEQ ID NO: 304 - DNA, 25 bp, synthetic construct
  source 1..25: mol_type = other DNA
agatgggtgatcagacccaacagca

SEQ ID NO: 305 - DNA, 26 bp, synthetic construct
  source 1..26: mol_type = other DNA
agatgggtgatcagacccaacagcag

SEQ ID NO: 306 - DNA, 27 bp, synthetic construct
  source 1..27: mol_type = other DNA
agatgggtgatcagacccaacagcagg

SEQ ID NO: 307 - DNA, 28 bp, synthetic construct
  source 1..28: mol_type = other DNA
agatgggtgatcagacccaacagcaggt

SEQ ID NO: 308 - DNA, 27 bp, synthetic construct
  source 1..27: mol_type = other DNA
aaaacctggtcgagctggacggcgacg

SEQ ID NO: 309 - DNA, 27 bp, synthetic construct
  source 1..27: mol_type = other DNA
aaaactgagcacccagtccgccctgag

SEQ ID NO: 310 - DNA, 27 bp, synthetic construct
  source 1..27: mol_type = other DNA
aaaacacgctggatagcctccaggcca

SEQ ID NO: 311 - DNA, 27 bp, synthetic construct
  source 1..27: mol_type = other DNA
aaaacattctctgctggatgacgtgag

SEQ ID NO: 312 - DNA, 27 bp, synthetic construct
  source 1..27: mol_type = other DNA
aaaatcatccagcagagaatggaaagt

SEQ ID NO: 313 - DNA, 27 bp, synthetic construct
  source 1..27: mol_type = other DNA
aaaaaacccagacacatagcaattcag

SEQ ID NO: 314 - DNA, 27 bp, synthetic construct
  source 1..27: mol_type = other DNA
aaaatactgaagaatggagagagaatt

SEQ ID NO: 315 - DNA, 27 bp, synthetic construct
  source 1..27: mol_type = other DNA
aaaagcctattcaccgattttgattct

SEQ ID NO: 316 - DNA, 27 bp, synthetic construct
  source 1..27: mol_type = other DNA
aaaattgattctcaaacaaatgtgtca

SEQ ID NO: 317 - DNA, 27 bp, synthetic construct
  source 1..27: mol_type = other DNA
aaaagccgtgtaccagctgagagactc

SEQ ID NO: 318 - DNA, 27 bp, synthetic construct
  source 1..27: mol_type = other DNA
aaaagattctgatgtgtatatcacaga

SEQ ID NO: 319 - DNA, 27 bp, synthetic construct
  source 1..27: mol_type = other DNA
aaaattgtgacacatttgtttgagaat

SEQ ID NO: 320 - DNA, 27 bp, synthetic construct
  source 1..27: mol_type = other DNA
aaaactcaggcggaggtgagcggaagg

SEQ ID NO: 321 - DNA, 27 bp, synthetic construct
  source 1..27: mol_type = other DNA
aaaacaacacatcggagagcttcgtgc

SEQ ID NO: 322 - DNA, 27 bp, synthetic construct
  source 1..27: mol_type = other DNA
aaaatccctggcccccaaggcgcagat

SEQ ID NO: 323 - DNA, 27 bp, synthetic construct
  source 1..27: mol_type = other DNA
aaaacacccaggccagccggccagttc

SEQ ID NO: 324 - DNA, 27 bp, synthetic construct
  source 1..27: mol_type = other DNA
aaaactagctgagctctcggacccctg

SEQ ID NO: 325 - DNA, 27 bp, synthetic construct
  source 1..27: mol_type = other DNA
aaaaactgtggggtggaggggacagat

SEQ ID NO: 326 - DNA, 27 bp, synthetic construct
  source 1..27: mol_type = other DNA
aaaagtaaggggggtaggggagctgcc

SEQ ID NO: 327 - DNA, 27 bp, synthetic construct
  source 1..27: mol_type = other DNA
aaaatggtgcctgatatagcttgacac

SEQ ID NO: 328 - DNA, 27 bp, synthetic construct
  source 1..27: mol_type = other DNA
aaaaaggaatatcgggaaaagacacat

SEQ ID NO: 329 - DNA, 27 bp, synthetic construct
  source 1..27: mol_type = other DNA
aaaactgagagactctaaatccagtga

SEQ ID NO: 330 - DNA, 27 bp, synthetic construct
  source 1..27: mol_type = other DNA
aaaatcactggatttagagtctctcag

SEQ ID NO: 331 - DNA, 27 bp, synthetic construct
  source 1..27: mol_type = other DNA
aaaaggacatgatctcatttccctctt

SEQ ID NO: 332 - DNA, 27 bp, synthetic construct
  source 1..27: mol_type = other DNA
aaaatttgtttgagaatcaaaatcggt

SEQ ID NO: 333 - DNA, 27 bp, synthetic construct
  source 1..27: mol_type = other DNA
aaaaattctgatgtgtatatcacagac

SEQ ID NO: 334 - DNA, 27 bp, synthetic construct
  source 1..27: mol_type = other DNA
aaaagagttatctgtagtgagatggct

SEQ ID NO: 335 - DNA, 27 bp, synthetic construct
  source 1..27: mol_type = other DNA
aaaagggggtaggggagctgcccaaat

SEQ ID NO: 336 - DNA, 27 bp, synthetic construct
  source 1..27: mol_type = other DNA
aaaattccagaaccggaggacaaagta

SEQ ID NO: 337 - DNA, 27 bp, synthetic construct
  source 1..27: mol_type = other DNA
aaaaggtgtggttccagaaccggagga

SEQ ID NO: 338 - DNA, 27 bp, synthetic construct
  source 1..27: mol_type = other DNA
aaaagttagcttgcactagactagctt

SEQ ID NO: 339 - DNA, 27 bp, synthetic construct
  source 1..27: mol_type = other DNA
aaaaacgcccccaccctaatcagaggc

SEQ ID NO: 340 - DNA, 27 bp, synthetic construct
  source 1..27: mol_type = other DNA
aaaaaaggtgaggaggtgggtggcaga

SEQ ID NO: 341 - DNA, 27 bp, synthetic construct
  source 1..27: mol_type = other DNA
aaaagtggccccaactgtgatgacctg

SEQ ID NO: 342 - DNA, 25 bp, synthetic construct
  source 1..25: mol_type = other DNA
aaaaaacccttcctggagcctgtga

SEQ ID NO: 343 - DNA, 25 bp, synthetic construct
  source 1..25: mol_type = other DNA
aaaagacacacacgtcctcactctc

SEQ ID NO: 344 - DNA, 25 bp, synthetic construct
  source 1..25: mol_type = other DNA
aaaacgctggatagcctccaggcca

SEQ ID NO: 345 - DNA, 24 bp, synthetic construct
  source 1..24: mol_type = other DNA
aaaagtgtaccagctgagagactc

SEQ ID NO: 346 - DNA, 25 bp, synthetic construct
  source 1..25: mol_type = other DNA
aaaacgtgtaccagctgagagactc

SEQ ID NO: 347 - DNA, 26 bp, synthetic construct
  source 1..26: mol_type = other DNA
aaaaccgtgtaccagctgagagactc

SEQ ID NO: 348 - DNA, 28 bp, synthetic construct
  source 1..28: mol_type = other DNA
aaaatgccgtgtaccagctgagagactc

SEQ ID NO: 349 - DNA, 24 bp, synthetic construct
  source 1..24: mol_type = other DNA
aaaagctgttgggtctgatcaccc

SEQ ID NO: 350 - DNA, 25 bp, synthetic construct
  source 1..25: mol_type = other DNA
aaaatgctgttgggtctgatcaccc

SEQ ID NO: 351 - DNA, 26 bp, synthetic construct
  source 1..26: mol_type = other DNA
aaaactgctgttgggtctgatcaccc

SEQ ID NO: 352 - DNA, 28 bp, synthetic construct
  source 1..28: mol_type = other DNA
aaaaacctgctgttgggtctgatcaccc

SEQ ID NO: 353 - DNA, 81 bp, synthetic construct
  source 1..81: mol_type = other DNA
cctctaatacgactcactatagcctttggaagtactaagaatttctactgttgtagataggtgaagttcgagggcgacgaa

SEQ ID NO: 354 - DNA, 81 bp, synthetic construct
  source 1..81: mol_type = other DNA
ttcgtcgccctcgaacttcacctatctacaacagtagaaattcttagtacttccaaaggctatagtgagtcgtattagagg

SEQ ID NO: 355 - DNA, 80 bp, synthetic construct
  source 1..80: mol_type = other DNA
cctctaatacgactcactataggctactaagcctttataatttctactattgtagataggtgaagttcgagggcgacgaa

SEQ ID NO: 356 - DNA, 80 bp, synthetic construct
  source 1..80: mol_type = other DNA
ttcgtcgccctcgaacttcacctatctacaatagtagaaattataaaggcttagtagcctatagtgagtcgtattagagg

SEQ ID NO: 357 - DNA, 80 bp, synthetic construct
  source 1..80: mol_type = other DNA
cctctaatacgactcactatagtctgaaagactatataatttctacttcgtgtagataggtgaagttcgagggcgacgaa

SEQ ID NO: 358 - DNA, 80 bp, synthetic construct
  source 1..80: mol_type = other DNA
ttcgtcgccctcgaacttcacctatctacacgaagtagaaattatatagtctttcagactatagtgagtcgtattagagg

SEQ ID NO: 359 - DNA, 31 bp, synthetic construct
  source 1..31: mol_type = other DNA
cggcattcctgctgaaccgctcttccgatct

SEQ ID NO: 360 - DNA, 30 bp, synthetic construct
  source 1..30: mol_type = other DNA
gatcggaagagcggttcagcaggaatgccg

SEQ ID NO: 361 - DNA, 55 bp, synthetic construct
  source 1..55: mol_type = other DNA
tcgtcggcagcgtcagatgtgtataagagacagctgctgaaccgctcttccgatc

SEQ ID NO: 362 - DNA, 60 bp, synthetic construct
  source 1..60: mol_type = other DNA
tcgtcggcagcgtcagatgtgtataagagacagtaagactgctgaaccgctcttccgatc

SEQ ID NO: 363 - DNA, 72 bp, synthetic construct
  source 1..72: mol_type = other DNA
tcgtcggcagcgtcagatgtgtataagagacaggctagacctaatgtgatctgctgaaccgctcttccgatc

SEQ ID NO: 364 - DNA, 63 bp, synthetic construct
  source 1..63: mol_type = other DNA
gtctcgtgggctcggagatgtgtataagagacagtctgcgttctgatttaatctgtatcaggc

SEQ ID NO: 365 - protein, 6 aa, synthetic construct
HHHHHH

SEQ ID NO: 366 - DNA, 20 bp, synthetic construct
  source 1..20: mol_type = other DNA
catcacgagcagctggtttc

SEQ ID NO: 367 - DNA, 20 bp, synthetic construct
  source 1..20: mol_type = other DNA
ctctaacctggcactgcgtc

SEQ ID NO: 368 - DNA, 20 bp, synthetic construct
  source 1..20: mol_type = other DNA
tggccagagtggaaatggaa

SEQ ID NO: 369 - DNA, 20 bp, synthetic construct
  source 1..20: mol_type = other DNA
cactgcctctgtcactctcg

SEQ ID NO: 370 - DNA, 21 bp, synthetic construct
  source 1..21: mol_type = other DNA
cctcacgtagaaggaagaggc

SEQ ID NO: 371 - DNA, 20 bp, synthetic construct
  source 1..20: mol_type = other DNA
aatggtgaccggcatctctg

SEQ ID NO: 372 - DNA, 20 bp, synthetic construct
  source 1..20: mol_type = other DNA
ccttatattcccagggccgg

SEQ ID NO: 373 - DNA, 20 bp, synthetic construct
  source 1..20: mol_type = other DNA
actttgatgtctgcaggcca

SEQ ID NO: 374 - DNA, 21 bp, synthetic construct
  source 1..21: mol_type = other DNA
atttcggactaccctgaggag

SEQ ID NO: 375 - DNA, 20 bp, synthetic construct
  source 1..20: mol_type = other DNA
tgctagacttgctgctcctt

SEQ ID NO: 376 - DNA, 20 bp, synthetic construct
  source 1..20: mol_type = other DNA
tgaacttcagctcctgcaca

SEQ ID NO: 377 - DNA, 24 bp, synthetic construct
  source 1..24: mol_type = other DNA
gcatcacaacaggcagagaatgtc

SEQ ID NO: 378 - DNA, 20 bp, synthetic construct
  source 1..20: mol_type = other DNA
tggcaatggataaggccgag

SEQ ID NO: 379 - DNA, 23 bp, synthetic construct
  source 1..23: mol_type = other DNA
ggtgctaggacatgcgaacttag

SEQ ID NO: 380 - DNA, 21 bp, synthetic construct
  source 1..21: mol_type = other DNA
tgtatttgtgcaagtgctgct

SEQ ID NO: 381 - DNA, 17 bp, synthetic construct
  source 1..17: mol_type = other DNA
tggggctcccatcctta

SEQ ID NO: 382 - DNA, 20 bp, synthetic construct
  source 1..20: mol_type = other DNA
agagatgccggtcaccattc

SEQ ID NO: 383 - DNA, 20 bp, synthetic construct
  source 1..20: mol_type = other DNA
aaggcacagtggatcatgca

SEQ ID NO: 384 - DNA, 20 bp, synthetic construct
  source 1..20: mol_type = other DNA
gagaaagggagtagaggcgg

SEQ ID NO: 385 - DNA, 18 bp, synthetic construct
  source 1..18: mol_type = other DNA
gagcctcagcccttccac

SEQ ID NO: 386 - DNA, 22 bp, synthetic construct
  source 1..22: mol_type = other DNA
ggaatctaccaccccaggctct

SEQ ID NO: 387 - DNA, 20 bp, synthetic construct
  source 1..20: mol_type = other DNA
tgaagggattgtgctggtgt

SEQ ID NO: 388 - DNA, 20 bp, synthetic construct
  source 1..20: mol_type = other DNA
tgcagttcccagtacgttcc

SEQ ID NO: 389 - DNA, 24 bp, synthetic construct
  source 1..24: mol_type = other DNA
tatgacgtcagggggaggcaagtc

SEQ ID NO: 390 - DNA, 31 bp, synthetic construct
  source 1..31: mol_type = other DNA
acgattagaaataatttctactgttgtagat

SEQ ID NO: 391 - DNA, 36 bp, synthetic construct
  source 1..36: mol_type = other DNA
ctttgaaagaatataatttctactgaaagtgtagat

SEQ ID NO: 392 - DNA, 36 bp, synthetic construct
  source 1..36: mol_type = other DNA
gtttaataataatacataatttctactattgtagat

SEQ ID NO: 393 - DNA, 36 bp, synthetic construct
  source 1..36: mol_type = other DNA
gtctataagactaatttaatttctactattgtagat

SEQ ID NO: 394 - DNA, 36 bp, synthetic construct
  source 1..36: mol_type = other DNA
gtttataaaccttatctaatttctactgttgtagat

SEQ ID NO: 395 - DNA, 35 bp, synthetic construct
  source 1..35: mol_type = other DNA
tctaaagctctttaagaatttctactttcgtagat

SEQ ID NO: 396 - DNA, 34 bp, synthetic construct
  source 1..34: mol_type = other DNA
ctaagaggctcaaataatttctactattgtagat

SEQ ID NO: 397 - DNA, 36 bp, synthetic construct
  source 1..36: mol_type = other DNA
cctttggaagtactaagaatttctactgttgtagat

SEQ ID NO: 398 - DNA, 36 bp, synthetic construct
  source 1..36: mol_type = other DNA
ggctataaaagccatataatttctactattgtagat

SEQ ID NO: 399 - DNA, 36 bp, synthetic construct
  source 1..36: mol_type = other DNA
gactattaagtcttttgaatttctactgttgtagat

SEQ ID NO: 400 - DNA, 35 bp, synthetic construct
  source 1..35: mol_type = other DNA
tctagaatatataggtaatttctacttatgtagat

SEQ ID NO: 401 - DNA, 37 bp, synthetic construct
  source 1..37: mol_type = other DNA
ggcaataagccatatacaatttctactgtatgtagat

SEQ ID NO: 402 - DNA, 36 bp, synthetic construct
  source 1..36: mol_type = other DNA
gtctgaaagactatataatttctacttcgtgtagat

SEQ ID NO: 403 - DNA, 36 bp, synthetic construct
  source 1..36: mol_type = other DNA
gtctacggaacgtctgtaatttctactgttgtagat

SEQ ID NO: 404 - DNA, 35 bp, synthetic construct
  source 1..35: mol_type = other DNA
tttaaacgaactattaaatttctactgttgtagat

SEQ ID NO: 405 - DNA, 36 bp, synthetic construct
  source 1..36: mol_type = other DNA
gtttaatacttatatataatttctactattgtagat

SEQ ID NO: 406 - DNA, 35 bp, synthetic construct
  source 1..35: mol_type = other DNA
aataataatcccttataatttctacttttgtagat

SEQ ID NO: 407 - DNA, 36 bp, synthetic construct
  source 1..36: mol_type = other DNA
gtctataagacgaactaaatttctactattgtagat

SEQ ID NO: 408 - DNA, 36 bp, synthetic construct
  source 1..36: mol_type = other DNA
ggctactaagcctttataatttctactattgtagat

SEQ ID NO: 409 - DNA, 36 bp, synthetic construct
  source 1..36: mol_type = other DNA
gtctataaagacgaatgaatttctactattgtagat

SEQ ID NO: 410 - DNA, 31 bp, synthetic construct
  source 1..31: mol_type = other DNA
cctgccgtgtaccagctgagagactctaaat

SEQ ID NO: 411 - DNA, 31 bp, synthetic construct
  source 1..31: mol_type = other DNA
cctgccgtgagactctgtcccggttgtgaga

SEQ ID NO: 412 - DNA, 27 bp, synthetic construct
  source 1..27: mol_type = other DNA
tttagagtctctcagctggtacacggc